(19) United States
(12) Patent Application Publication
(10) Pub. No.: US 2024/0109881 A1
(43) Pub. Date: Apr. 4, 2024

(54) Heteroaryl Compounds as Ligand Directed Degraders of IRAK4

(71) Applicants: Bristol-Myers Squibb Company, Princeton, NJ (US); Celgene Corporation, Summit, NJ (US)

(72) Inventors: Timothy Rasmusson, Sudbury, MA (US); Geraint Davies, Arlington, MA (US); Paul Gormisky, Belmont, MA (US); Rulin Ma, Winchester, MA (US); Michael Ellis, Milton, MA (US); Lingbowei Hu, Cambridge, MA (US); Tony Siu, Wellesley, MA (US); Farid van der Mei, Arlington, MA (US); Harry Hager, Cambridge, MA (US); Yilin Meng, Newton, MA (US)

(73) Assignees: Bristol-Myers Squibb Company, Princeton, NJ (US); Celgene Corporation, Summit, NJ (US)

(21) Appl. No.: 18/223,768

(22) Filed: Jul. 19, 2023

Related U.S. Application Data
(60) Provisional application No. 63/390,888, filed on Jul. 20, 2022.

(51) Int. Cl. C07D 417/14; C07D 239/22; C07D 401/10; C07D 401/12; C07D 401/14; C07D 413/14; C07D 471/04; C07D 487/04; C07D 493/04; C07D 519/00

(52) U.S. Cl. CPC C07D 417/14; C07D 239/22; C07D 401/10; C07D 401/12; C07D 401/14; C07D 413/14; C07D 471/04; C07D 487/04; C07D 493/04; C07D 519/00

(57) ABSTRACT

Provided herein are compounds and compositions thereof for modulating IRAK4. In some embodiments, the compounds and compositions are provided for treatment of inflammatory or autoimmune diseases.

### CROSS-REFERENCE TO RELATED APPLICATIONS

This application claims priority to U.S. Provisional Application No. 63/390,888, filed on Jul. 20, 2022, which is incorporated herein by reference in its entirety for any purpose.

### FIELD

The present disclosure relates generally to compounds, compositions, and methods for their preparation and use of the compounds and compositions for treating inflammatory or autoimmune diseases.

### BACKGROUND

The recruitment of immune cells to sites of injury involves the concerted interactions of a large number of soluble mediators. Several cytokines appear to play key roles in these processes, including interleukin-1 (IL-1). IL-1 produces proinflammatory responses and contributes to the tissue degeneration observed in chronic inflammatory conditions. IL-1 has also been implicated in the process of bone resorption and adipose tissue regulation. Thus, IL-1 plays a key role in a large number of pathological conditions including rheumatoid arthritis, inflammatory bowel disease, multiple sclerosis, diabetes, obesity, cancer, and sepsis.

IL-1 treatment of cells induces the formation of a complex consisting of the two IL-1 receptor chains, IL-1R1 and IL-1RAcP, and the resulting heterodimer recruits an adaptor molecule designated as MyD88, which binds to IL-1 receptor associated kinase (IRAK) (Wesche et al., J. Biol. Chem. 1999, 274, 19403-19410; O'Neill et al., J. Leukoc. Biol. 1998, 63, 650-657; Auron, Cytokine Growth Factor Rev. 1998, 9:221-237; and O'Neill, Biochem. Soc. Trans. 2000, 28, 557-563). Four members of the IRAK family have been identified: IRAK1, IRAK2, IRAK3, and IRAK4. These proteins are characterized by a typical N-terminal death domain that mediates interaction with MyD88-family adaptor proteins and a centrally located kinase domain. Of the four members in the mammalian IRAK family, IRAK-4 is considered to be the “master IRAK.” IRAK-4 is a serine/threonine kinase that plays an essential role in signal transduction by Toll/IL-1 receptors (TIRs). Under overexpression conditions, all IRAKs can mediate the activation of nuclear factor-kappa B and stress-induced mitogen activated protein kinase (MAPK)-signaling cascades. Studies have shown that IRAK4 kinase activity is essential for cytokine production, activation of MAPKs, and induction of NF-kappa B regulated genes in response to TLR ligands (Koziczak-Holbro M. et al., J. Biol. Chem. 2007, 282, 13552-13560). Given the central role of IRAK4 in Toll-like/IL-1R signaling and immunological protection, compounds that modulate the function of IRAK4 may be useful in treating inflammatory, cell proliferative, and immune-related conditions and diseases associated with IRAK-mediated signal transduction such as rheumatoid arthritis, inflammatory bowel disease, multiple sclerosis, diabetes, obesity, allergic disease, psoriasis, asthma, graft rejection, cancer and sepsis.

Protein degradation is a highly regulated and essential process that maintains cellular homeostasis. Selective identification and removal of damaged, misfolded, or excess proteins is achieved through the ubiquitin-proteasome pathway (UPP). The UPP is central to the regulation of almost all cellular processes. Ubiquitination of the protein is accomplished by an E3 ubiquitin ligase that binds to a protein and adds ubiquitin molecules to the protein, thus marking the protein for proteasome degradation.

Harnessing the UPP for therapeutic use has received significant interest (Zhou et al., Mol. Cell 2000, 6, 751-756). One promising therapy uses proteolysis targeting chimeras, commonly referred to as PROTACs, to effect removal of unwanted proteins by protein degradation (Scheepstra et al., Comp. Struct. Biotech. J. 2019, 17, 160-176). PROTACS are ligand directed degraders that bring together an E3 ligase and a target protein that is to be degraded. These bivalent molecules usually consist of an E3 ligase ligand connected through a linker moiety to small molecule that binds to the target protein. A PROTAC positions the E3 ligase at the appropriate distance and orientation to the target protein, allowing the latter to be ubiquitinated. The ubiquitinated target protein is subsequently recognized by the proteasome, where it is degraded.

Accordingly, in one aspect, provided herein are compounds that target IRAK4 for degradation.

### SUMMARY

Described herein, in certain embodiments, are compounds and compositions thereof for degrading IRAK4. In various embodiments, the compounds and compositions thereof may be used in treatment of inflammatory or autoimmune diseases.

The present embodiments can be understood more fully by reference to the detailed description and examples, which are intended to exemplify non-limiting embodiments.

Embodiment A1 is a compound of Formula (I′):

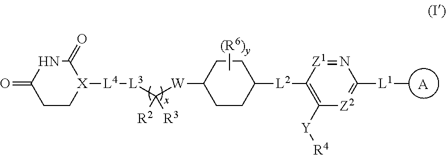

or a pharmaceutically acceptable salt thereof, wherein:
Ring A is phenyl, monocyclic 5- to 6-membered heteroaryl, or fused bicyclic 9- to 10-membered heteroaryl or heterocyclyl, wherein the heteroaryl and heterocyclyl contain 1-4 heteroatoms independently selected from N, O, and S, each of which is optionally substituted by 1-3 R⁰ groups;
each R⁰ is independently selected from halo, —CN, —NH₂, —NH(C₁-C₆ alkyl), C₁-C₆ alkyl, C₃-C₆ cycloalkyl, -(6- to 10-membered bridged heterocyclylene)-, and C₁-C₆ alkoxy, wherein the heterocyclylene contains 1-3 heteroatoms selected from N and O, or two R⁰ groups are taken together to form an oxo group;
L¹ is —NH— or a bond; L² is —NHC(O)—, —C(O)NH—, —SO₂NH—, —NHSO₂—, or —(C₁-C₆ alkylene)_{z}(5-membered heteroarylene)-, wherein the heteroarylene contains 1-3 heteroatoms selected from N, O, and S;
L³ is —NR⁹ (C₁-C₆ alkylene)NR⁹—, —NR⁹C(O)(C₁-C₆ alkylene)_{z}(4- to 7-membered heterocyclylene)-, -(4- to 7-membered heterocyclylene)CR¹¹R¹²—, -(4- to 7-membered heterocyclylene)(CO)_{z}—, -(4- to 7-membered heterocyclylene)(NR⁹)_{z}—, —(NR⁹)_{z}(4- to 7-membered heterocyclylene)(C₁-C₆ alkylene)_{z}-, —NR⁹ (C₁-C₆ alkylene)_{z}(4- to 7-membered heterocyclylene)-, —NR⁹C(O)(phenylene)NR⁹—, —(C₁-C₆ alkylene)_{z}(4- to 7-membered heterocyclylene)(C₁-C₆ alkylene)_{z}-, —O(C₁-C₆ alkylene)_{z}(4- to 7-membered heterocyclylene)(C₁-C₆ alkylene)_{z}-, -(6- to 10-membered bridged heterocyclylene)(C₁-C₆ alkylene)_{z}-, -(7- to 10-membered fused bicyclic heterocyclylene)(C₁-C₆ alkylene)_{z}-, or —(O)_{z}(6- to 10-membered spiro heterocyclylene)(C₁-C₆ alkylene)_{z}-, wherein the heterocyclylene contains 1-3 heteroatoms selected from N and O and is optionally substituted by 1-5 R⁷ groups;
L⁴ is

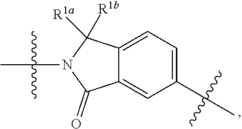

phenylene, —N(H)(phenylene), 5- to 6-membered heteroarylene, —N(H)(5- to 6-membered heteroarylene)-, 8- to 10-membered fused bicyclic heteroarylene, or 5- to 6-membered heterocyclylene, wherein the phenylene, heteroarylene, or heterocyclylene is optionally substituted by 1-4 R¹⁰ groups, and wherein the heteroarylene and heterocyclylene contain 1-3 heteroatoms selected from N, S, and O;
R^{1a} and R^{1b} are each H or are taken together to form an oxo group;
R² and R³ are independently H, C₁-C₆ alkyl, or halo, or R² and R³ are taken together to form an oxo group;
or R³ and R¹¹ are taken together to form a C₃-C₆ cycloalkylene group;
Y is NH, O, or a bond;
R⁴ is C₃-C₆ cycloalkyl, C₁-C₆ alkylene-(C₃-C₆ cycloalkyl), 4- to 6-membered heterocyclyl, C₁-C₆ alkylene-(4- to 6-membered heterocyclyl), 5- to 6-membered heteroaryl, C₁-C₆ alkylene-(5- to 6-membered heteroaryl), C₁-C₆ alkyl, C₁-C₆ haloalkyl, C₁-C₆ alkyl-OH, or C₁-C₆ alkyl-CN, wherein the heterocyclyl and heteroaryl contain 1-3 heteroatoms selected from N and O, and wherein the cycloalkyl, heterocyclyl, or heteroaryl is optionally substituted by 1-5 R⁸ groups;
W is O, —NR⁵—, or a bond;
R⁵ is H or C₁-C₆ alkyl;
each R⁶ is independently C₁-C₆ alkyl, halo, or —OH, or two R⁶ groups are taken together to form a bridging C1-C₃ alkylene group;
each R⁷ is independently C₁-C₆ alkyl, halo, C₁-C₆ haloalkyl, or —OH, or two R⁷ groups are taken together to form an oxo group;
each R⁸ is independently —SO₂(C₁-C₆ alkyl), —C(O)(C₁-C₆ alkyl), C₁-C₆ alkyl, C₁-C₆ haloalkyl, halo, —CN, or —OH;
each R⁹ is independently H or C₁-C₆ alkyl;
each R¹⁰ is independently C₁-C₆ alkoxy, C₁-C₆ alkyl, halo, or —OH, or two R¹⁰ groups are taken together to form an oxo group;
each R¹¹ and R¹² is independently H, halo, C₃-C₆ cycloalkyl, —OH, —NH(C₁-C₆ alkyl), C₁-C₆ haloalkyl, or C₁-C₆ alkyl;
or R¹¹ and R³ are taken together to form a C₃-C₆ cycloalkylene group;
x is 0 or 1;
y is 0, 1, 2, 3, 4, or 5;
each z is independently 0 or 1;
X is N or CR¹³;
R¹³ is H, halo, —OH, or C₁-C₆ alkyl;
Z¹ is CH or N; and
Z² is CH or N,
   provided that Z¹ and Z² are not both N.

Embodiment A2 is the compound of embodiment A1, or a pharmaceutically acceptable salt thereof, wherein the compound is of Formula (I):

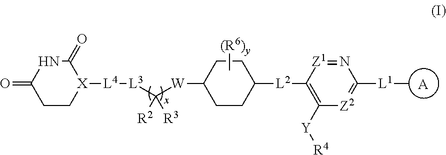

wherein:
Ring A is phenyl, monocyclic 5- to 6-membered heteroaryl, or fused bicyclic 9- to 10-membered heteroaryl or heterocyclyl, wherein the heteroaryl and heterocyclyl contain 1-4 heteroatoms independently selected from N, O, and S, each of which is optionally substituted by 1-3 R⁰ groups;
each R⁰ is independently selected from halo, —CN, —NH₂, —NH(C₁-C₆ alkyl), C₁-C₆ alkyl, C₃-C₆ cycloalkyl, and C₁-C₆ alkoxy, or two R⁰ groups are taken together to form an oxo group;
L¹ is —NH— or a bond;
L² is —NHC(O)—, —C(O)NH—, —SO₂NH—, —NHSO₂—, or 5-membered heteroarylene containing 1-3 heteroatoms selected from N, O, and S;
L³ is —NR⁹ (C₁-C₆ alkylene)NR⁹—, —NR⁹C(O)(C₁-C₆ alkylene)_{z}(4- to 7-membered heterocyclylene)-, -(4- to 7-membered heterocyclylene)CR¹¹R¹²—, -(4- to 7-membered heterocyclylene)(CO)_{z}—, -(4- to 7-membered heterocyclylene)(NR⁹)_{z}—, —(NR⁹)_{z}(4- to 7-membered heterocyclylene)(C₁-C₆ alkylene)_{z}-, —NR⁹ (C₁-C₆ alkylene)_{z}(4- to 7-membered heterocyclylene)-, —NR⁹C(O)(phenylene)NR⁹—, —(C₁-C₆ alkylene)_{z}(4- to 7-membered heterocyclylene)-, —O(C₁-C₆ alkylene)_{z}(4- to 7-membered heterocyclylene)-, -(6- to 10-membered bridged heterocyclylene)(C₁-C₆ alkylene)_{z}-, -(9- to 10-membered fused bicyclic heterocyclylene)(C₁-C₆ alkylene)_{z}-, or —(O)_{z}(6- to 10-membered spiro heterocyclylene)(C₁-C₆ alkylene)_{z}-, wherein the heterocyclylene contains 1-3 heteroatoms selected from N and O and is optionally substituted by 1-5 R⁷ groups;
L⁴ is

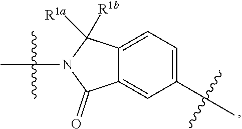

phenylene, 5- to 6-membered heteroarylene, or 5- to 6-membered heterocyclylene, wherein the phenylene, heteroarylene, or heterocyclylene is optionally substituted by 1-4 R¹⁰ groups, and wherein the heteroarylene and heterocyclylene contain 1-3 heteroatoms selected from N and O;
R^{1a} and R^{1b} are each H or are taken together to form an oxo group;
R² and R³ are independently H, C₁-C₆ alkyl, or halo, or R² and R³ are taken together to form an oxo group;
or R³ and R¹¹ are taken together to form a C₃-C₆ cycloalkylene group;
Y is NH or O;
R⁴ is C₃-C₆ cycloalkyl, C₁-C₆ alkylene-(C₃-C₆ cycloalkyl), 4- to 6-membered heterocyclyl, C₁-C₆ alkylene-(4- to 6-membered heterocyclyl), 5- to 6-membered heteroaryl, C₁-C₆ alkylene-(5- to 6-membered heteroaryl), C₁-C₆ alkyl, C₁-C₆ haloalkyl, C₁-C₆ alkyl-OH, or C₁-C₆ alkyl-CN, wherein the heterocyclyl and heteroaryl contain 1-3 heteroatoms selected from N and O, and wherein the cycloalkyl, heterocyclyl, or heteroaryl is optionally substituted by 1-5 R⁸ groups;
W is O, or a bond;
R⁵ is H or C₁-C₆ alkyl;
each R⁶ is independently C₁-C₆ alkyl, halo, or —OH, or two R⁶ groups are taken together to form a bridging C1-C₃ alkylene group;
each R⁷ is independently C₁-C₆ alkyl, halo, C₁-C₆ haloalkyl, or —OH, or two R⁷ groups are taken together to form an oxo group;
each R⁸ is independently —SO₂(C₁-C₆ alkyl), —C(O)(C₁-C₆ alkyl), C₁-C₆ alkyl, C₁-C₆ haloalkyl, halo, —CN, or —OH;
each R⁹ is independently H or C₁-C₆ alkyl;
each R¹⁰ is independently C₁-C₆ alkoxy, C₁-C₆ alkyl, halo, or —OH, or two R¹⁰ groups are taken together to form an oxo group;
each R¹¹ and R¹² is independently H, halo, C₃-C₆ cycloalkyl, —OH, —NH(C₁-C₆ alkyl), C₁-C₆ haloalkyl, or C₁-C₆ alkyl;
or R¹¹ and R³ are taken together to form a C₃-C₆ cycloalkylene group;
x is 0 or 1;
y is 0, 1, 2, 3, 4, or 5;
each z is independently 0 or 1;
X is N or CR¹³;
R¹³ is H, halo, or C₁-C₆ alkyl;
Z¹ is CH or N; and
Z² is CH or N,
   provided that Z¹ and Z² are not both N.

Embodiment A3 is the compound of embodiment A1 or A2, or a pharmaceutically acceptable salt thereof, wherein Ring A is:
(i) phenyl optionally substituted by 1-3 R⁰ groups; and
each R⁰ is independently selected from halo, —CN, —NH₂, —NH(C₁-C₃ alkyl), C₁-C₃ alkyl, C₃-C₆ cycloalkyl, and C₁-C₃ alkoxy;
(ii) a monocyclic 6-membered heteroaryl containing 1-2 heteroatoms independently selected from N and O, and optionally substituted by 1-3 R⁰ groups; and
each R⁰ is independently selected from halo, —CN, —NH₂, —NH(C₁-C₃ alkyl), C₁-C₃ alkyl, C₃-C₆ cycloalkyl, and C₁-C₃ alkoxy; or
(iii) a fused bicyclic 9-membered heteroaryl or heterocyclyl containing 2-4 heteroatoms independently selected from N, O, and S, and optionally substituted by 1-3 R⁰ groups; and
each R⁰ is independently selected from halo, —CN, —NH₂, —NH(C₁-C₃ alkyl), C₁-C₃ alkyl, C₃-C₆ cycloalkyl, -(6- to 8-membered bridged heterocyclylene)-, and C₁-C₃ alkoxy, wherein the heterocyclylene contains 1-3 heteroatoms selected from N and O, or two R⁰ groups are taken together to form an oxo group.

Embodiment A4 is the compound of embodiment A3, or a pharmaceutically acceptable salt thereof, wherein Ring A is:

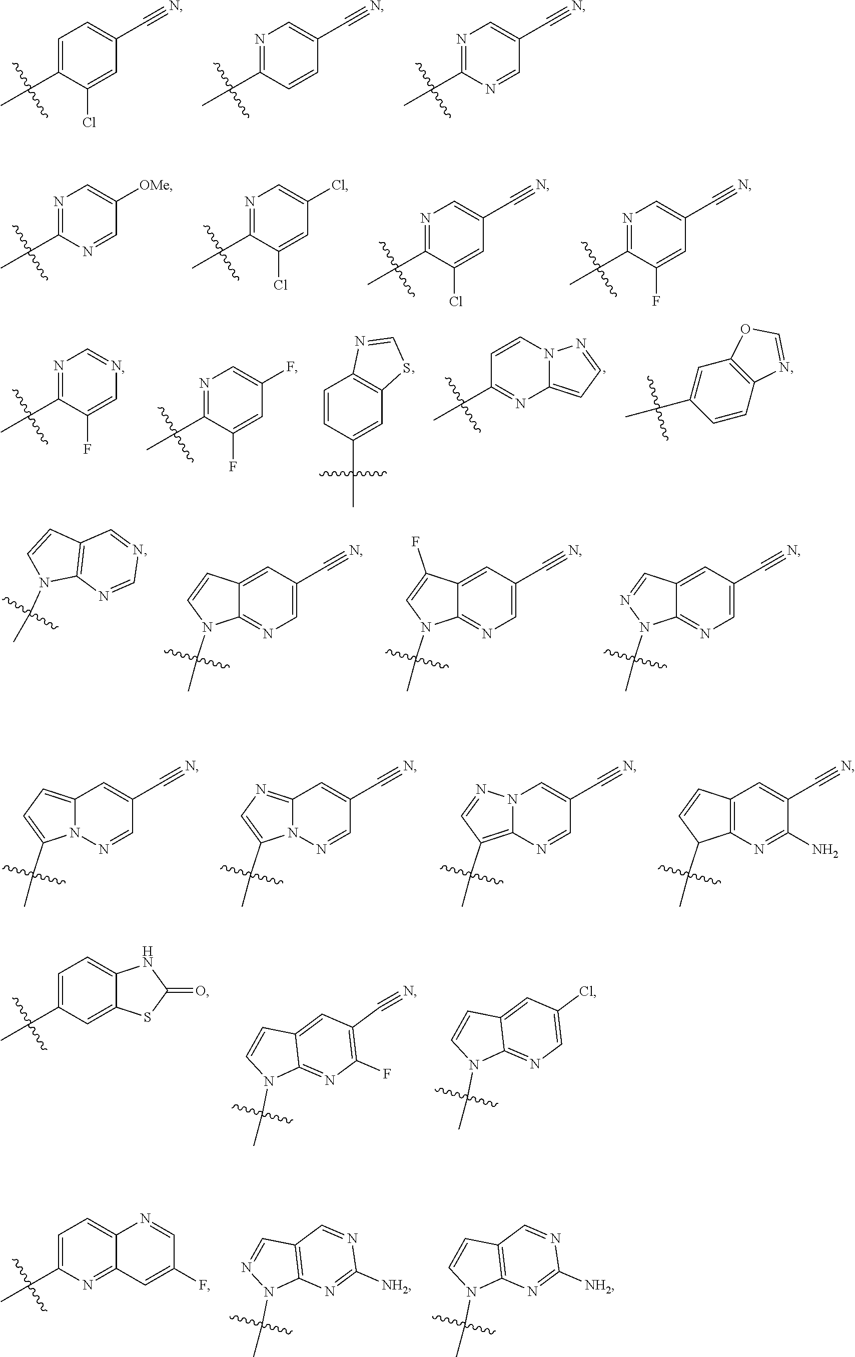

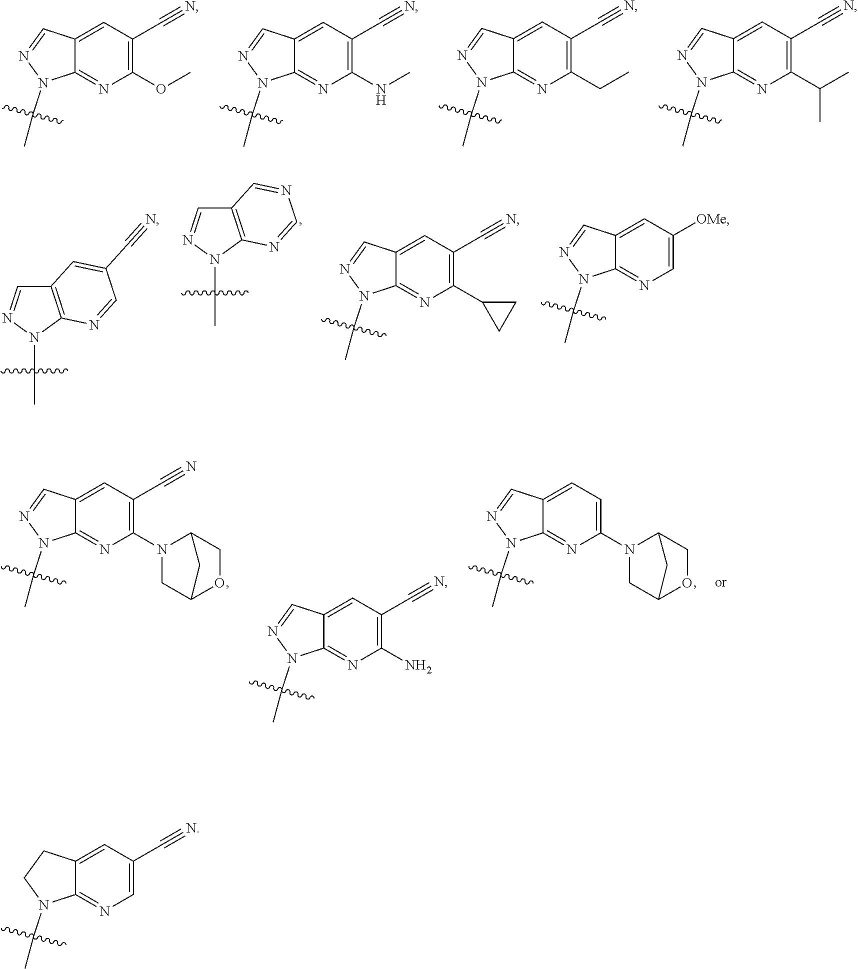

Embodiment A5 is the compound of any one of embodiments A1-A4, or a pharmaceutically acceptable salt thereof, wherein:
L² is —NHC(O)— or —(C₁-C₃ alkylene)_{z}(5-membered heteroarylene)-, wherein the heteroarylene contains 1-3 heteroatoms selected from N and O.

Embodiment A6 is the compound of embodiment A5, or a pharmaceutically acceptable salt thereof, wherein:
L² is —NHC(O)—,

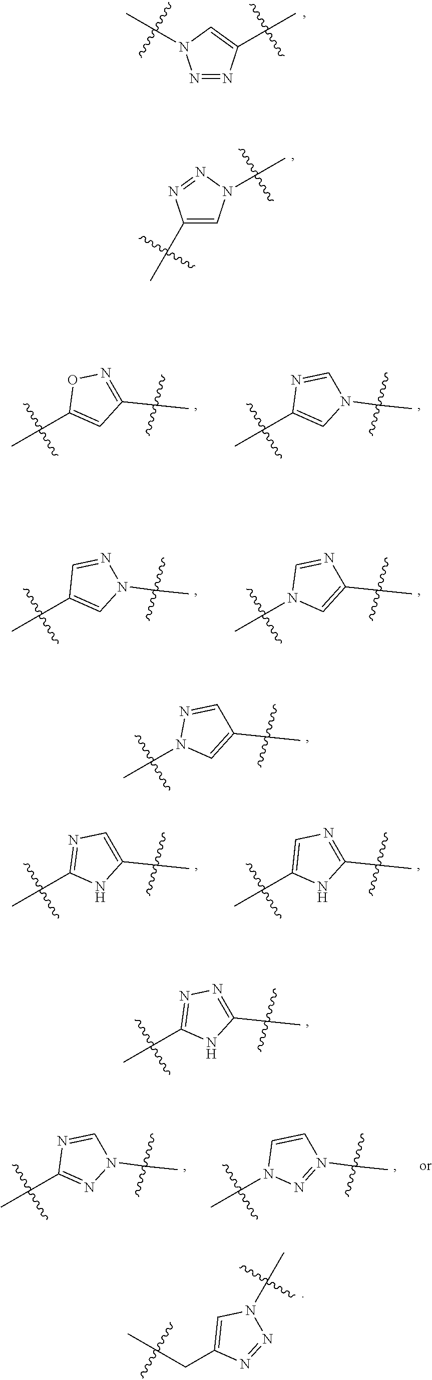

Embodiment A7 is the compound of any one of embodiments A1-A6, or a pharmaceutically acceptable salt thereof, wherein:
L⁴ is

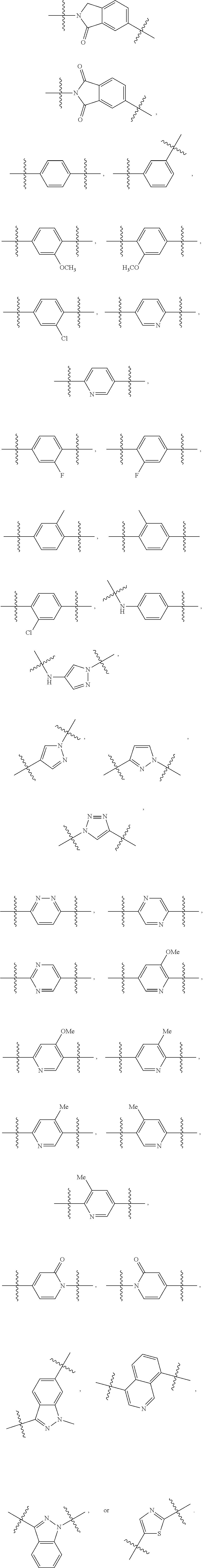

Embodiment A8 is the compound of any one of embodiments A1-A7, or a pharmaceutically acceptable salt thereof, wherein:

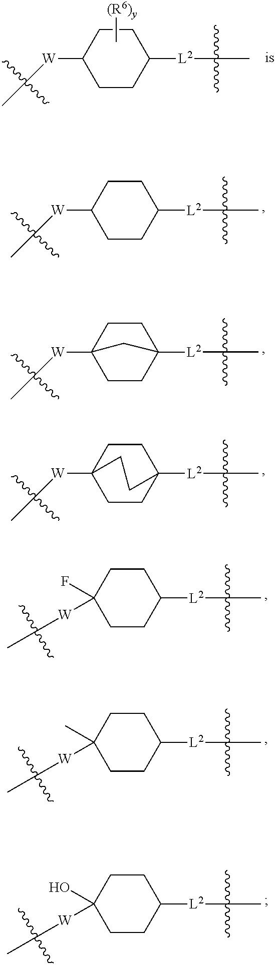

W is O, —NR⁵—, or a bond; and
R⁵ is H or C₁-C₃ alkyl.

Embodiment A9 is the compound of any one of embodiments A1-A8, or a pharmaceutically acceptable salt thereof, wherein:
X is N.

Embodiment A10 is the compound of any one of embodiments A1-A8, or a pharmaceutically acceptable salt thereof, wherein:
X is CR¹³; and
R¹³ is H, halo, —OH, or C₁-C₃ alkyl.

Embodiment A11 is the compound of any one of embodiments A1-A10, or a pharmaceutically acceptable salt thereof, wherein:
Y is NH;
R⁴ is C₃-C₆ cycloalkyl, C₁-C₃ alkylene-(C₃-C₆ cycloalkyl), 4- to 6-membered heterocyclyl, C₁-C₃ alkylene-(4- to 6-membered heterocyclyl), 5- to 6-membered heteroaryl, C₁-C₃ alkylene-(5- to 6-membered heteroaryl), C₁-C₆ alkyl, C₁-C₆ haloalkyl, C₁-C₆ alkyl-OH, or C₁-C₆ alkyl-CN, wherein the heterocyclyl and heteroaryl contain 1 or 2 heteroatoms selected from N and O, and wherein the cycloalkyl, heterocyclyl, or heteroaryl is optionally substituted by 1-2 R⁸ groups; and
each R⁸ is independently —SO₂(C₁-C₃ alkyl), —C(O)(C₁-C₃ alkyl), C₁-C₃ alkyl, C₁-C₃ haloalkyl, halo, —CN, or —OH.

Embodiment A12 is the compound of any one of embodiments A1-A11, or a pharmaceutically acceptable salt thereof, wherein:
R⁴ is methyl, ethyl, n-propyl, isopropyl, tert-butyl, —CH₂CH(CH₃)₂, —CH₂CF₃, —CH₂CH₂F, —CH₂CF₂CH₃, —CH(CH₃)CF₃, —CH₂CH₂CF₃, —CH(CH₃)CH₂OH, —CH₂C(CH₃)₂OH, —CH₂CN, —CH(CH₃)CN, —C(CH₃)₂CN, —CH(CH₂CH₃)CN, —CH₂CH(CH₃)CN,

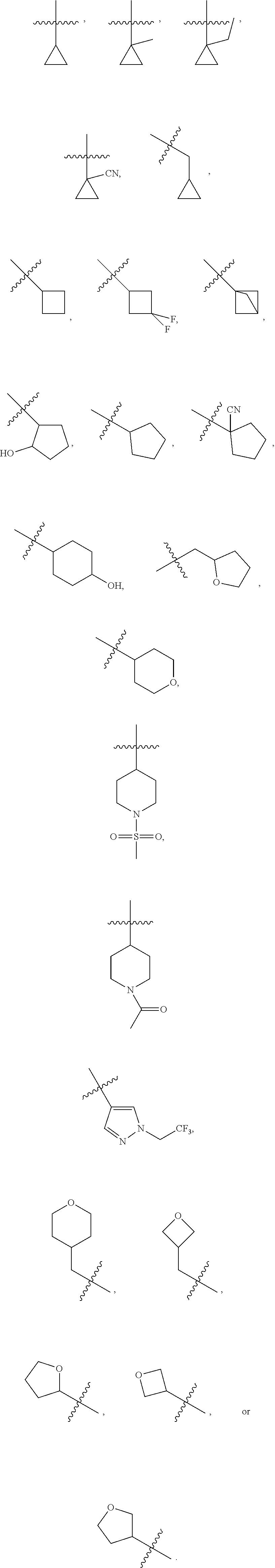

Embodiment A13 is the compound of any one of embodiments A1-A12, or a pharmaceutically acceptable salt thereof, wherein:
L³ is —NR⁹ (C₁-C₃ alkylene)NR⁹—, —NR⁹C(O)(C₁-C₃ alkylene)_{z}(4- to 7-membered heterocyclylene)-, -(4- to 7-membered heterocyclylene)CR¹¹R¹²—, -(4- to 7-membered heterocyclylene)(CO)_{z}—, -(4- to 7-membered heterocyclylene)(NR⁹)_{z}—, —(NR⁹)_{z}(4- to 7-membered heterocyclylene)(C₁-C₃ alkylene)_{z}-, —NR⁹ (C₁-C₃ alkylene)_{z}(4- to 7-membered heterocyclylene)-, —NR⁹C(O)(phenylene)NR⁹—, —(C₁-C₃ alkylene)_{z}(4- to 7-membered heterocyclylene)(C₁-C₃ alkylene)_{z}-, —O(C₁-C₃ alkylene)_{z}(4- to 7-membered heterocyclylene)(C₁-C₃ alkylene)_{z}-, -(6- to 10-membered bridged heterocyclylene)(C₁-C₃ alkylene)_{z}-, -(7- to 10-membered fused bicyclic heterocyclylene)(C₁-C₆ alkylene)_{z}-, or —(O)_{z}(6- to 10-membered spiro heterocyclylene)(C₁-C₃ alkylene)_{z}-, wherein the heterocyclylene contains 1-3 heteroatoms selected from N and O and is optionally substituted by 1 or 2 R⁷ groups;
each z is independently 0 or 1;
each R⁹ is independently H or C₁-C₃ alkyl;
each R⁷ is independently C₁-C₃ alkyl, halo, C₁-C₃ haloalkyl, or —OH, or two R⁷ groups are taken together to form an oxo group; and
each R¹¹ and R¹² is independently H or —CH₃.

Embodiment A14 is the compound of embodiment A13, or a pharmaceutically acceptable salt thereof, wherein:
L³ is

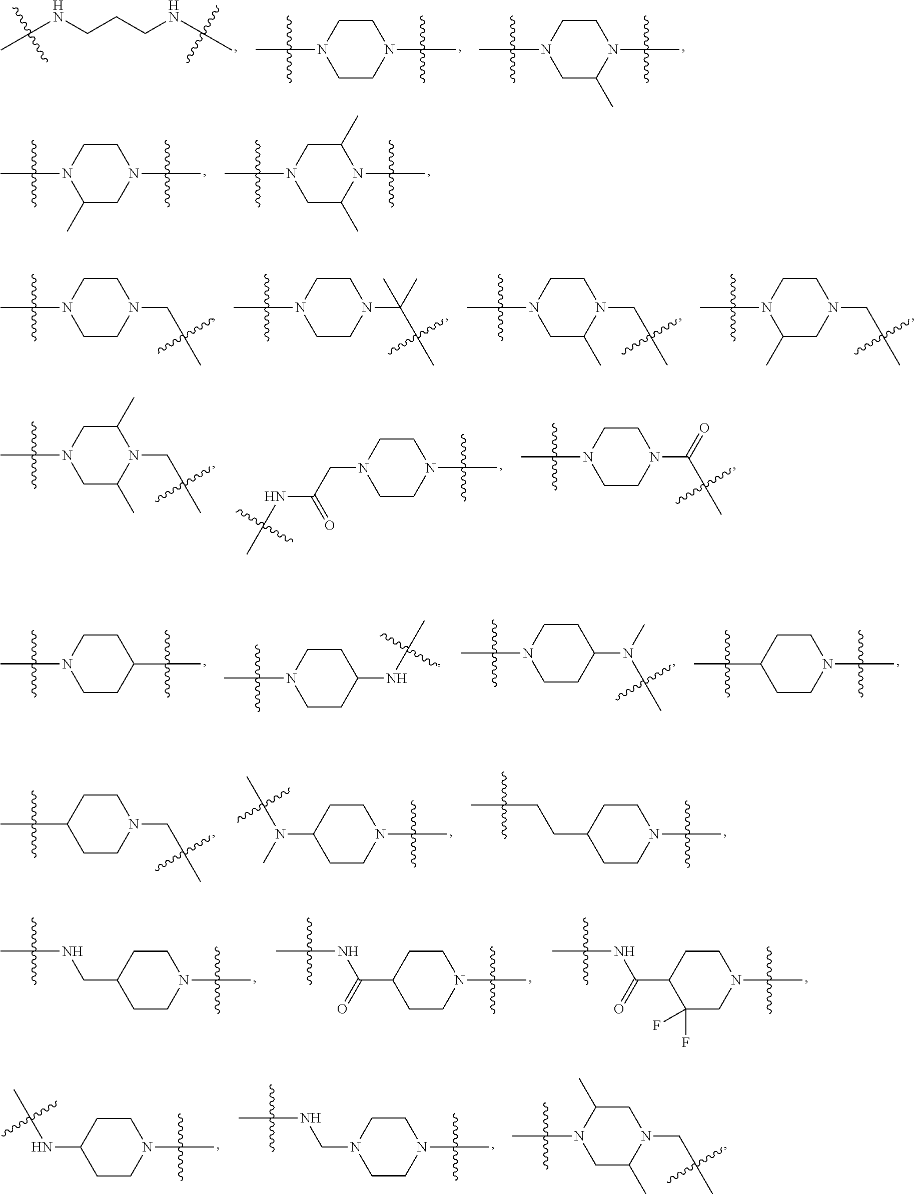

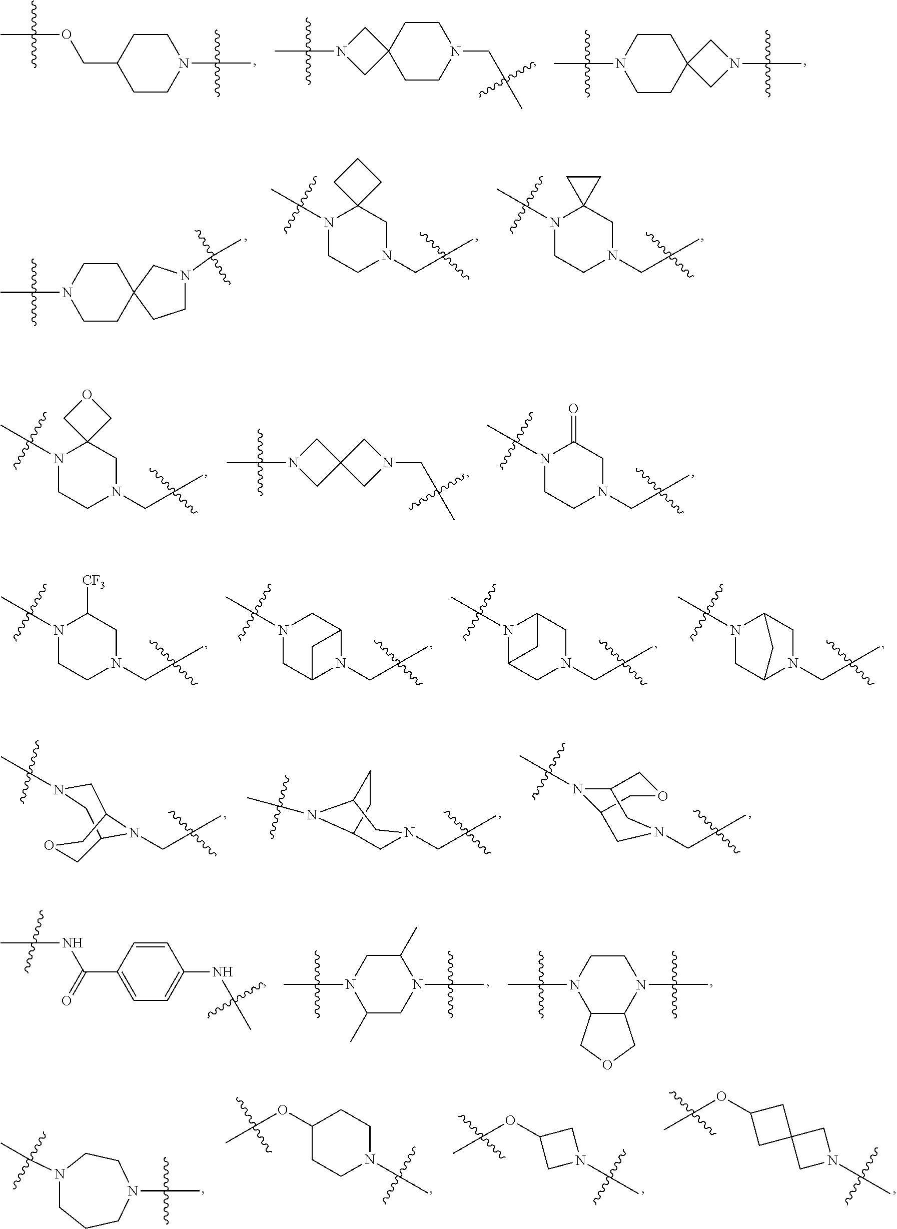

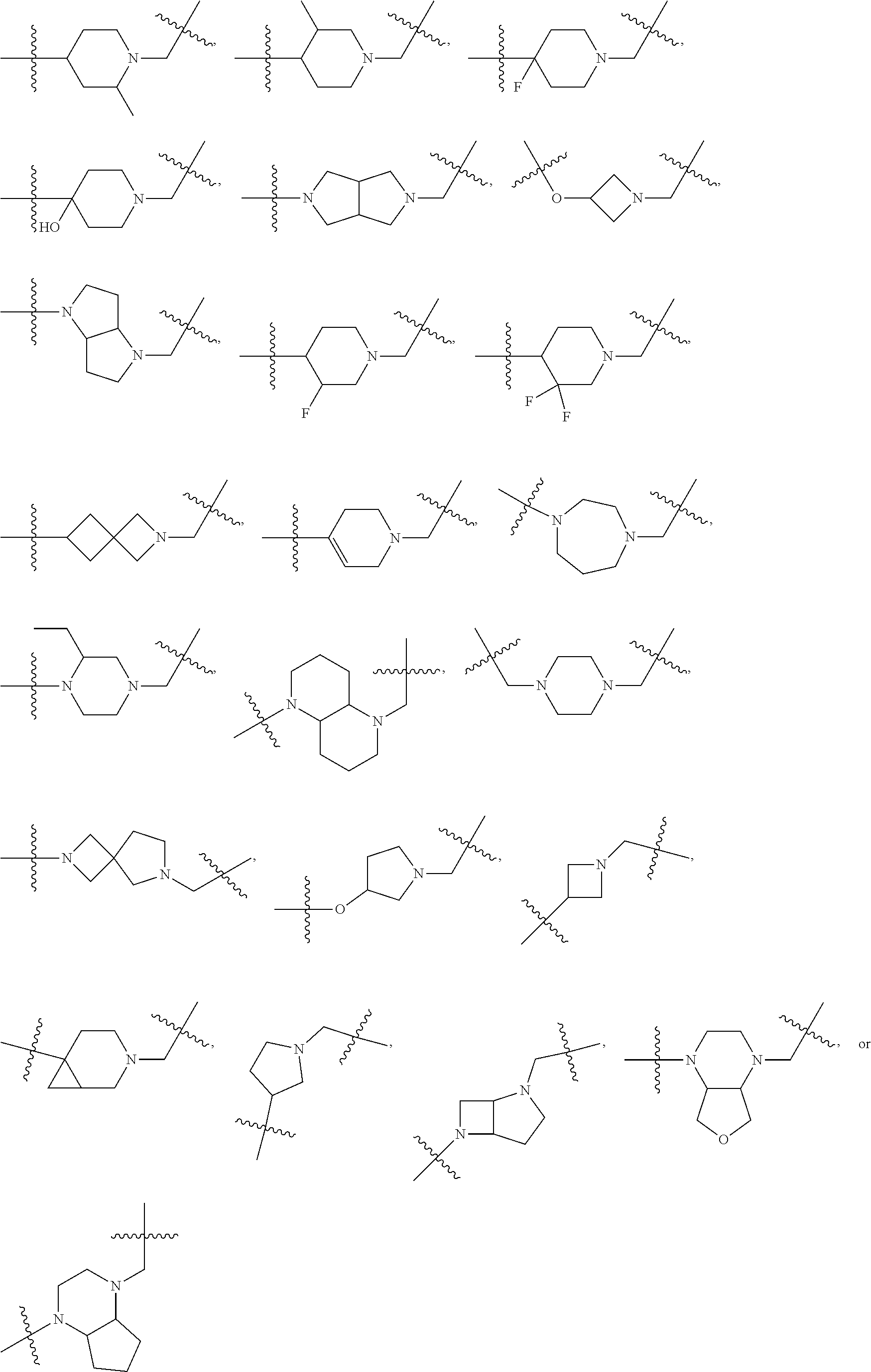

Embodiment A15 is the compound of any one of embodiments A1-A14, or a pharmaceutically acceptable salt thereof, wherein the compound is of Formula (Ia) or (Ia′):

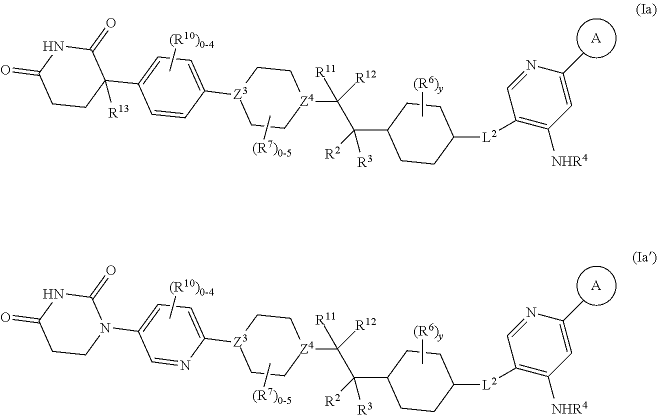

wherein:
Ring A is a fused bicyclic 9- to 10-membered heteroaryl containing 2-4 heteroatoms independently selected from N, O, and S, optionally substituted by 1-3 R⁰ groups;
R⁴ is C₁-C₆ alkyl, C₁-C₆ haloalkyl, C₁-C₆ alkyl-OH, or C₁-C₆ alkyl-CN; and
Z³ and Z⁴ are independently N or CH, provided that at least one of Z³ and Z⁴ is N.

Embodiment A16 is the compound of embodiment A15, or a pharmaceutically acceptable salt thereof, wherein the compound is of Formula (If) or (If′):

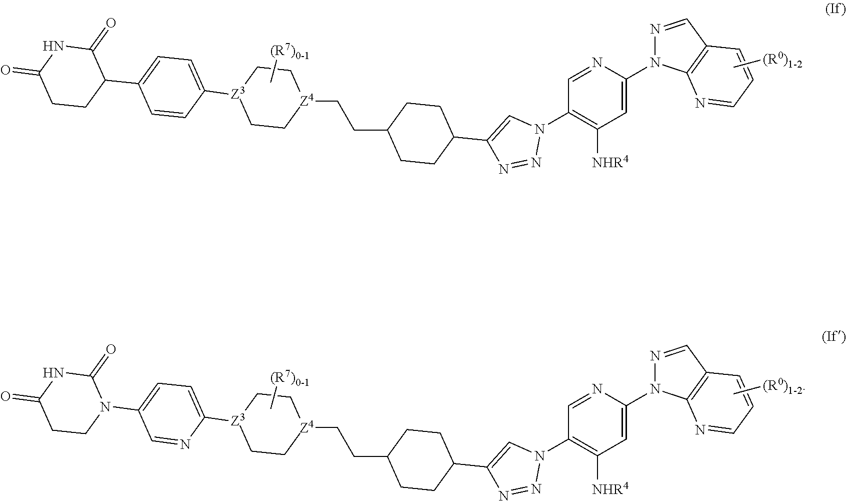

Embodiment A17 is a compound selected from the compounds of Table 1 or a pharmaceutically acceptable salt thereof.

Embodiment A18 is a pharmaceutical composition comprising the compound of any one of embodiments A1-A17, or a pharmaceutically acceptable salt thereof, and a pharmaceutically acceptable excipient.

Embodiment A19 is a method of modulating interleukin-1 (IL1) receptor-associated kinase 4 (IRAK4) activity comprising contacting IRAK4 with an effective amount of the compound of any one of embodiments A1-A17, or a pharmaceutically acceptable salt thereof, or the pharmaceutical composition of embodiment A18.

Embodiment A20 is a method of treating an inflammatory or autoimmune disease in a subject in need thereof, comprising administering to the subject an effective amount of the compound of any one of embodiments A1-A17, or a pharmaceutically acceptable salt thereof, or the pharmaceutical composition of embodiment A18, optionally wherein the inflammatory or autoimmune disease is atopic dermatitis, asthma, lupus, rheumatoid arthritis, familial mediterranean fever, psoriasis, generalized pustular psoriasis, cryoprin-associated periodic syndrome, hidradenitis suppurativa, Bechet's syndrome, or familial cold autoinflammatory syndrome.

### DETAILED DESCRIPTION

### Definitions

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thFereof Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “approximately” mean±20%, ±10%, ±5%, or ±1% of the indicated range, value, or structure, unless otherwise indicated.

An “alkyl” group is a saturated, partially saturated, or unsaturated straight chain or branched non-cyclic hydrocarbon having from 1 to 10 carbon atoms (C₁-C₁₀ alkyl), typically from 1 to 8 carbons (C₁-C₈ alkyl) or, in some embodiments, from 1 to 6 (C₁-C₆ alkyl), 1 to 3 (C₁-C₃ alkyl), or 2 to 6 (C₂-C₆ alkyl) carbon atoms. In some embodiments, the alkyl group is a saturated alkyl group. Representative saturated alkyl groups include -methyl, -ethyl, -n-propyl, -n-butyl, -n-pentyl and -n-hexyl; while saturated branched alkyls include -isopropyl, -sec-butyl, -isobutyl, tert-butyl, -isopentyl, -neopentyl, tertpentyl, -2-methylpentyl, -3-methylpentyl, -4-methylpentyl, -2,3-dimethylbutyl and the like. In some embodiments, an alkyl group is an unsaturated alkyl group, also termed an alkenyl or alkynyl group. An “alkenyl” group is an alkyl group that contains one or more carbon-carbon double bonds. An “alkynyl” group is an alkyl group that contains one or more carbon-carbon triple bonds. Examples of unsaturated alkyl groups include, but are not limited to, vinyl, allyl, CH═CH(CH₃), —CH═C(CH₃)₂, —C(CH₃)═CH₂, —C(CH₃)═CH(CH₃), —C(CH₂CH₃)═CH₂, —C≡C(CH₃), —C≡C(CH₂CH₃), —CH₂C≡CH, CH₂CC(CH₃) and CH₂CC(CH₂CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen; hydroxy; alkoxy;

cycloalkyloxy, aryloxy, heterocyclyloxy, heteroaryloxy, heterocycloalkyloxy, cycloalkylalkyloxy, aralkyloxy, heterocyclylalkyloxy, heteroarylalkyloxy, heterocycloalkylalkyloxy; oxo (═O); amino, alkylamino, cycloalkylamino, arylamino, heterocyclylamino, heteroarylamino, heterocycloalkylamino, cycloalkylalkylamino, aralkylamino, heterocyclylalkylamino, heteroaralkylamino, heterocycloalkylalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

“Alkyl-OH” refers to an unbranched or branched alkyl group as defined above, wherein one or more hydrogen atoms are replaced by —OH. For example, “C₁-C₆ alkyl-OH” refers to a C₁-C₆ alkyl which is substituted by one or more —OH groups. An alkyl-OH may contain multiple hydroxy groups that are attached to the same carbon atom or to multiple carbon atoms.

“Alkyl-CN” refers to an unbranched or branched alkyl group as defined above, wherein one or more hydrogen atoms are replaced by —CN. For example, “C₁-C₆ alkyl-CN” refers to a C₁-C₆ alkyl which is substituted by one or more —CN groups. An alkyl-CN may contain multiple cyano groups that are attached to the same carbon atom or to multiple carbon atoms.

An “alkoxy” group is —O-(alkyl), wherein alkyl is defined above.

A “cycloalkyl” group is a saturated, or partially saturated cyclic alkyl group of from 3 to 10 carbon atoms (C₃-C₁₀ cycloalkyl) having a single cyclic ring or multiple condensed or bridged rings that can be optionally substituted. In some embodiments, the cycloalkyl group has 3 to 8 ring carbon atoms (C₃-C₈ cycloalkyl), whereas in other embodiments the number of ring carbon atoms ranges from 3 to 5 (C₃-C₅ cycloalkyl), 3 to 6 (C₃-C₆ cycloalkyl), or 3 to 7 (C₃-C₇ cycloalkyl). In some embodiments, the cycloalkyl groups are saturated cycloalkyl groups. Such saturated cycloalkyl groups include, by way of example, single ring structures such as cyclopropyl, cyclobutyl, cyclopentyl, cyclohexyl, cycloheptyl, cyclooctyl, 1methylcyclopropyl, 2methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. In other embodiments, the cycloalkyl groups are unsaturated cycloalkyl groups. Examples of unsaturared cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

An “aryl” group is an aromatic carbocyclic group of from 6 to 14 carbon atoms (C₆-C₁₄ aryl) having a single ring (e.g., phenyl) or multiple condensed rings (e.g., naphthyl or anthryl). In some embodiments, aryl groups contain 6-14 carbons (C₆-C₁₄ aryl), and in others from 6 to 12 (C₆-C₁₂ aryl) or even 6 to 10 carbon atoms (C₆-C₁₀ aryl) in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “halogen” or “halo” is fluorine, chlorine, bromine or iodine.

“Haloalkyl” refers to an alkyl radical, as defined above, that is substituted by one or more halo radicals, as defined above, e.g., trifluoromethyl, difluoromethyl, trichloromethyl, 2,2,2trifluoroethyl, 1,2difluoroethyl, 3bromo2fluoropropyl, 1,2dibromoethyl, and the like. In some embodiments, the haloalkyl group has one to six carbon atoms and is substituted by one or more halo radicals (C₁-C₆ haloalkyl), or the haloalkyl group has one to three carbon atoms and is substituted by one or more halo radicals (C₁-C₃ haloalkyl). The halo radicals may be all the same or the halo radicals may be different. Unless specifically stated otherwise, a haloalkyl group is optionally substituted.

A “heteroaryl” group is an aromatic ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1Hpyrrolo[2,3b]pyridyl), indazolyl, benzimidazolyl (e.g., 1Hbenzo[d]imidazolyl), imidazopyridyl (e.g., azabenzimidazolyl or 1Himidazo[4,5b]pyridyl), pyrazolopyridyl, triazolopyridyl, benzotriazolyl (e.g., 1Hbenzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4dihydroisoquinolin-1(2H)-onyl), tetrahydroquinolinyl, quinoxalinyl, and quinazolinyl groups. A heteroaryl group can be substituted or unsubstituted.

A “heterocyclyl” is a non-aromatic cycloalkyl in which one to four of the ring carbon atoms are independently replaced with a heteroatom selected from O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocycloalkyl group can be substituted or unsubstituted. Heterocyclyl groups encompass saturated and partially saturated ring systems. Further, the term heterocyclyl is intended to encompass any non-aromatic ring containing at least one heteroatom, which ring may be fused to an aryl or heteroaryl ring, regardless of the attachment to the remainder of the molecule. The phrase also includes bridged polycyclic ring systems containing a heteroatom. Representative examples of a heterocyclyl group include, but are not limited to, aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4dioxaspiro[4.5]decanyl, homopiperazinyl, quinuclidyl, or tetrahydropyrimidin-2(1H)-one. Representative substituted heterocyclyl groups may be monosubstituted or substituted more than once, such as, but not limited to, pyridyl or morpholinyl groups, which are 2-, 3-, 4-, 5-, or 6substituted, or disubstituted with various substituents such as those listed below.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

Certain commonly used alternative chemical names may be used. For example, a divalent group such as a divalent “alkyl” group, a divalent “phenyl” group, a divalent “heteroaryl” group, a divalent “heterocyclyl” group etc., may also be referred to as an “alkylene” group, a “phenylene” group, a “heteroarylene” group, or a “heterocyclylene” group, respectively.

Embodiments of the disclosure are meant to encompass pharmaceutically acceptable salts, tautomers, isotopologues, and stereoisomers of the compounds provided herein, such as the compounds of Formula (I′) or (I).

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds of Formula (I′) or (I) include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (Nmethyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and ptoluenesulfonic acid. Specific non-toxic acids include hydrochloric, hydrobromic, maleic, phosphoric, sulfuric, and methanesulfonic acids. Examples of specific salts thus include hydrochloride, formic, and mesylate salts. Others are well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereoisomerically pure” means one stereoisomer of a particular compound that is substantially free of other stereoisomers of that compound. For example, a stereoisomerically pure compound having one chiral center will be substantially free of the opposite enantiomer of the compound. A stereoisomerically pure compound having two chiral centers will be substantially free of other diastereomers of the compound. A typical stereoisomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds disclosed herein can have chiral centers and can occur as racemates, individual enantiomers or diastereomers, and mixtures thereof. All such isomeric forms are included within the embodiments disclosed herein, including mixtures thereof.

The use of stereoisomerically pure forms of the compounds disclosed herein, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (WileyInterscience, New York, 1981); Wilen, S. H., et al., Tetrahedron 33:2725 (1977); Eliel, E. L., Stereochemistry of Carbon Compounds (McGrawHill, NY, 1962); Wilen, S. H., Tables of Resolving Agents and Optical Resolutions p. 268 (E. L. Eliel, Ed., Univ. of Notre Dame Press, Notre Dame, IN, 1972); Todd, M., Separation Of Enantiomers: Synthetic Methods (Wiley-VCH Verlag GmbH & Co. KGaA, Weinheim, Germany, 2014); Toda, F., Enantiomer Separation: Fundamentals and Practical Methods (Springer Science & Business Media, 2007); Subramanian, G. Chiral Separation Techniques: A Practical Approach (John Wiley & Sons, 2008); Ahuja, S., Chiral Separation Methods for Pharmaceutical and Biotechnological Products (John Wiley & Sons, 2011).

It should also be noted the compounds disclosed herein can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

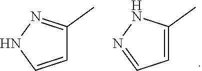

As readily understood by one skilled in the art, a wide variety of functional groups and other structures may exhibit tautomerism and all tautomers of compounds of Formula (I′) or (I) are within the scope of the present disclosure.

It should also be noted the compounds disclosed herein can contain unnatural proportions of atomic isotopes at one or more of the atoms. For example, the compounds may be radiolabeled with radioactive isotopes, such as for example tritium (³H), iodine-125 (¹²⁵I), sulfur35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds disclosed herein, for example, the isotopologues are deuterium, carbon-13, and/or nitrogen-15 enriched compounds. As used herein, “deuterated”, means a compound wherein at least one hydrogen (H) has been replaced by deuterium (indicated by D or 2H), that is, the compound is enriched in deuterium in at least one position.

It is understood that, independently of stereoisomerical or isotopic composition, each compound disclosed herein can be provided in the form of any of the pharmaceutically acceptable salts discussed herein. Equally, it is understood that the isotopic composition may vary independently from the stereoisomerical composition of each compound referred to herein. Further, the isotopic composition, while being restricted to those elements present in the respective compound or salt thereof disclosed herein, may otherwise vary independently from the selection of the pharmaceutically acceptable salt of the respective compound.

It should be noted that if there is a discrepancy between a depicted structure and a name for that structure, the depicted structure is to be accorded more weight.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In one embodiment, the disorder is a neurodegenerative disease, as described herein, or a symptom thereof.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In one embodiment, the disorder is a neurodegenerative disease, as described herein, or symptoms thereof.

The term “effective amount” in connection with a compound disclosed herein means an amount capable of treating or preventing a disorder, disease or condition, or symptoms thereof, disclosed herein.

The term “subject” or “patient” as used herein include an animal, including, but not limited to, an animal such a cow, monkey, horse, sheep, pig, chicken, turkey, quail, cat, dog, mouse, rat, rabbit or guinea pig, in one embodiment a mammal, in another embodiment a human. In one embodiment, a subject is a human having or at risk for having an SIPS mediated disease, or a symptom thereof.

Although various features of the invention may be described in the context of a single embodiment, the features may also be provided separately or in any suitable combination. Conversely, although the invention may be described herein in the context of separate embodiments for clarity, the invention may also be implemented in a single embodiment. Compounds In one aspect, provided herein is a compound of Formula (I′):

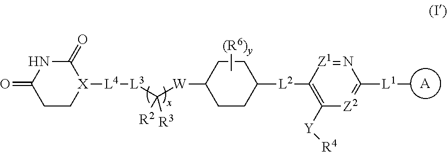

or a pharmaceutically acceptable salt thereof, wherein: Ring A is phenyl, monocyclic 5- to 6-membered heteroaryl, or fused bicyclic 9- to 10-membered heteroaryl or heterocyclyl, wherein the heteroaryl and heterocyclyl contain 1-4 heteroatoms independently selected from N, O, and S, each of which is optionally substituted by 1-3 R⁰ groups;
each R⁰ is independently selected from halo, —CN, —NH₂, —NH(C₁-C₆ alkyl), C₁-C₆ alkyl, C₃-C₆ cycloalkyl, -(6- to 10-membered bridged heterocyclylene)-, and C₁-C₆ alkoxy, wherein the heterocyclylene contains 1-3 heteroatoms selected from N and O, or two R⁰ groups are taken together to form an oxo group; L¹ is —NH— or a bond; L² is —NHC(O)—, —C(O)NH—, —SO₂NH—, —NHSO₂—, or —(C₁-C₆ alkylene)_{z}(5-membered heteroarylene)-, wherein the heteroarylene contains 1-3 heteroatoms selected from N, O, and
S; L³ is —NR⁹ (C₁-C₆ alkylene)NR⁹—, —NR⁹C(O)(C₁-C₆ alkylene)_{z}(4- to 7-membered heterocyclylene)-, -(4- to 7-membered heterocyclylene)CR¹¹R¹²—, -(4- to 7-membered heterocyclylene)(CO)_{z}—, -(4- to 7-membered heterocyclylene)(NR⁹)_{z}—, —(NR⁹)_{z}(4- to 7-membered heterocyclylene)(C₁-C₆ alkylene)_{z}-, —NR⁹ (C₁-C₆ alkylene)_{z}(4- to 7-membered heterocyclylene)-, —NR⁹C(O)(phenylene)NR⁹—, —(C₁-C₆ alkylene)_{z}(4- to 7-membered heterocyclylene)(C₁-C₆ alkylene)_{z}-, —O(C₁-C₆ alkylene)_{z}(4- to 7-membered heterocyclylene)(C₁-C₆ alkylene)_{z}-, -(6- to 10-membered bridged heterocyclylene)(C₁-C₆ alkylene)_{z}-, -(7- to 10-membered fused bicyclic heterocyclylene)(C₁-C₆ alkylene)_{z}-, or —(O)_{z}(6- to 10-membered spiro heterocyclylene)(C₁-C₆ alkylene)_{z}-, wherein the heterocyclylene contains 1-3 heteroatoms selected from N and O and is optionally substituted by 1-5 R⁷ groups;

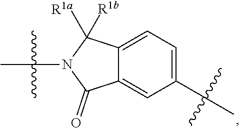

L⁴ is phenylene, —N(H)(phenylene), 5- to 6-membered heteroarylene, —N(H)(5- to 6-membered heteroarylene)-, 8- to 10-membered fused bicyclic heteroarylene, or 5- to 6-membered heterocyclylene, wherein the phenylene, heteroarylene, or heterocyclylene is optionally substituted by 1-4 R¹⁰ groups, and wherein the heteroarylene and heterocyclylene contain 1-3 heteroatoms selected from N, S, and O; R^{1a} and R^{1b} are each H or are taken together to form an oxo group; R² and R³ are independently H, C₁-C₆ alkyl, or halo, or R² and R³ are taken together to form an oxo group;
or R³ and R⁴ are taken together to form a C₃-C₆ cycloalkylene group; Y is NH, 0, or a bond;

R⁴ is C₃-C₆ cycloalkyl, C₁-C₆ alkylene-(C₃-C₆ cycloalkyl), 4- to 6-membered heterocyclyl, C₁-C₆ alkylene-(4- to 6-membered heterocyclyl), 5- to 6-membered heteroaryl, C₁-C₆ alkylene-(5- to 6-membered heteroaryl), C₁-C₆ alkyl, C₁-C₆ haloalkyl, C₁-C₆ alkyl-OH, or C₁-C₆ alkyl-CN, wherein the heterocyclyl and heteroaryl contain 1-3 heteroatoms selected from N and O, and wherein the cycloalkyl, heterocyclyl, or heteroaryl is optionally substituted by 1-5 R⁸ groups;
W is O, —NR⁵—, or a bond; R⁵ is H or C₁-C₆ alkyl;
each R⁶ is independently C₁-C₆ alkyl, halo, or —OH, or two R⁶ groups are taken together to form a bridging C1-C₃ alkylene group;
each R⁷ is independently C₁-C₆ alkyl, halo, C₁-C₆ haloalkyl, or —OH, or two R⁷ groups are taken together to form an oxo group;
each R⁸ is independently —SO₂(C₁-C₆ alkyl), —C(O)(C₁-C₆ alkyl), C₁-C₆ alkyl, C₁-C₆ haloalkyl, halo, —CN, or —OH;
each R⁹ is independently H or C₁-C₆ alkyl;
each R¹⁰ is independently C₁-C₆ alkoxy, C₁-C₆ alkyl, halo, or —OH, or two R¹⁰ groups are taken together to form an oxo group;
each R¹¹ and R¹² is independently H, halo, C₃-C₆ cycloalkyl, —OH, —NH(C₁-C₆ alkyl), C₁-C₆ haloalkyl, or C₁-C₆ alkyl;
or R¹¹ and R³ are taken together to form a C₃-C₆ cycloalkylene group;
x is 0 or 1;
y is 0, 1, 2, 3, 4, or 5;
each z is independently 0 or 1;
X is N or CR¹³;
R¹³ is H, halo, —OH, or C₁-C₆ alkyl;
Z¹ is CH or N; and
Z² is CH or N,
provided that Z¹ and Z² are not both N.

In a further aspect, provided herein is a compound of Formula (I):

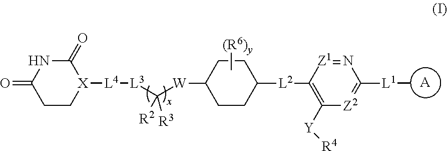

or a pharmaceutically acceptable salt thereof, wherein:
Ring A is phenyl, monocyclic 5- to 6-membered heteroaryl, or fused bicyclic 9- to 10-membered heteroaryl or heterocyclyl, wherein the heteroaryl and heterocyclyl contain 1-4 heteroatoms independently selected from N, O, and S, each of which is optionally substituted by 1-3 R⁰ groups;
each R⁰ is independently selected from halo, —CN, —NH₂, —NH(C₁-C₆ alkyl), C₁-C₆ alkyl, C₃-C₆ cycloalkyl, and C₁-C₆ alkoxy, or two R⁰ groups are taken together to form an oxo group;
L¹ is —NH— or a bond;
L² is —NHC(O)—, —C(O)NH—, —SO₂NH—, —NHSO₂—, or 5-membered heteroarylene containing 1-3 heteroatoms selected from N, O, and S;
L³ is —NR⁹ (C₁-C₆ alkylene)NR⁹—, —NR⁹C(O)(C₁-C₆ alkylene)_{z}(4- to 7-membered heterocyclylene)-, -(4- to 7-membered heterocyclylene)CR¹¹R¹²—, -(4- to 7-membered heterocyclylene)(CO)_{z}—, -(4- to 7-membered heterocyclylene)(NR⁹)_{z}—, —(NR⁹)_{z}(4- to 7-membered heterocyclylene)(C₁-C₆ alkylene)_{z}-, —NR⁹ (C₁-C₆ alkylene)_{z}(4- to 7-membered heterocyclylene)-, —NR⁹C(O)(phenylene)NR⁹—, —(C₁-C₆ alkylene)_{z}(4- to 7-membered heterocyclylene)-, —O(C₁-C₆ alkylene)_{z}(4- to 7-membered heterocyclylene)-, -(6- to 10-membered bridged heterocyclylene)(C₁-C₆ alkylene)_{z}-, -(9- to 10-membered fused bicyclic heterocyclylene)(C₁-C₆ alkylene)_{z}-, or —(O)_{z}(6- to 10-membered spiro heterocyclylene)(C₁-C₆ alkylene)_{z}-, wherein the heterocyclylene contains 1-3 heteroatoms selected from N and O and is optionally substituted by 1-5 R⁷ groups;
L⁴ is

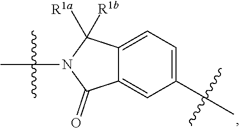

phenylene, 5- to 6-membered heteroarylene, or 5- to 6-membered heterocyclylene, wherein the phenylene, heteroarylene, or heterocyclylene is optionally substituted by 1-4 R¹⁰ groups, and wherein the heteroarylene and heterocyclylene contain 1-3 heteroatoms selected from N and O;
R^{1a} and R^{1b} are each H or are taken together to form an oxo group;
R² and R³ are independently H, C₁-C₆ alkyl, or halo, or R² and R³ are taken together to form an oxo group;
or R³ and R⁴ are taken together to form a C₃-C₆ cycloalkylene group; Y is NH or O;
R⁴ is C₃-C₆ cycloalkyl, C₁-C₆ alkylene-(C₃-C₆ cycloalkyl), 4- to 6-membered heterocyclyl, C₁-C₆ alkylene-(4- to 6-membered heterocyclyl), 5- to 6-membered heteroaryl, C₁-C₆ alkylene-(5- to 6-membered heteroaryl), C₁-C₆ alkyl, C₁-C₆ haloalkyl, C₁-C₆ alkyl-OH, or C₁-C₆ alkyl-CN, wherein the heterocyclyl and heteroaryl contain 1-3 heteroatoms selected from N and O, and wherein the cycloalkyl, heterocyclyl, or heteroaryl is optionally substituted by 1-5 R⁸ groups;
W is O, —NR⁵—, or a bond;
R⁵ is H or C₁-C₆ alkyl;
each R⁶ is independently C₁-C₆ alkyl, halo, or —OH, or two R⁶ groups are taken together to form a bridging C1-C₃ alkylene group;
each R⁷ is independently C₁-C₆ alkyl, halo, C₁-C₆ haloalkyl, or —OH, or two R⁷ groups are taken together to form an oxo group;
each R⁸ is independently —SO₂(C₁-C₆ alkyl), —C(O)(C₁-C₆ alkyl), C₁-C₆ alkyl, C₁-C₆ haloalkyl, halo, —CN, or —OH;
each R⁹ is independently H or C₁-C₆ alkyl;
each R¹⁰ is independently C₁-C₆ alkoxy, C₁-C₆ alkyl, halo, or —OH, or two R¹⁰ groups are taken together to form an oxo group;
each R¹¹ and R¹² is independently H, halo, C₃-C₆ cycloalkyl, —OH, —NH(C₁-C₆ alkyl), C₁-C₆ haloalkyl, or C₁-C₆ alkyl;
or R¹¹ and R³ are taken together to form a C₃-C₆ cycloalkylene group;
x is 0 or 1;
y is 0, 1, 2, 3, 4, or 5;
each z is independently 0 or 1;
X is N or CR¹³;
R¹³ is H, halo, or C₁-C₆ alkyl;
Z¹ is CH or N; and
Z² is CH or N,
provided that Z¹ and Z² are not both N.

In some embodiments, Z¹ is CH or N; and Z² is CH or N, provided that Z¹ and Z² are not both N. In some embodiments, Z¹ and Z² are each CH. In some embodiments, Z¹ is CH, and Z² is N. In some embodiments, Z¹ is N, and Z² is CH.

In some embodiments, Ring A is phenyl, monocyclic 5- to 6-membered heteroaryl, or fused bicyclic 9- to 10-membered heteroaryl or heterocyclyl, wherein the heteroaryl and heterocyclyl contain 1-4 heteroatoms independently selected from N, O, and S, each of which is optionally substituted by 1-3 R⁰ groups.

In some embodiments, Ring A is phenyl optionally substituted by 1-3 R⁰ groups. In some embodiments, Ring A is phenyl optionally substituted by 1-2 R⁰ groups. In some embodiments, Ring A is phenyl optionally substituted by 1 R⁰ group. In some embodiments, Ring A is phenyl optionally substituted by 2 R⁰ groups. In some embodiments, Ring A is phenyl optionally substituted by 3 R⁰ groups. In some embodiments, Ring A is unsubstituted phenyl.

In some embodiments, Ring A is monocyclic 5- to 6-membered heteroaryl optionally substituted by 1-3 R⁰ groups, wherein the heteroaryl contains 1˜4 heteroatoms independently selected from N, O, and S. In some embodiments, Ring A is monocyclic 5- to 6-membered heteroaryl optionally substituted by 1-2 R⁰ groups, wherein the heteroaryl contains 1˜4 heteroatoms independently selected from N, O, and S. In some embodiments, Ring A is monocyclic 5- to 6-membered heteroaryl optionally substituted by 1 R⁰ group, wherein the heteroaryl contains 1˜4 heteroatoms independently selected from N, O, and S. In some embodiments, Ring A is monocyclic 5- to 6-membered heteroaryl optionally substituted by 2 R⁰ groups, wherein the heteroaryl contains 1˜4 heteroatoms independently selected from N, O, and S. In some embodiments, Ring A is monocyclic 5- to 6-membered heteroaryl optionally substituted by 3 R⁰ groups, wherein the heteroaryl contains 1˜4 heteroatoms independently selected from N, O, and S. In some embodiments, Ring A is unsubstituted monocyclic 5- to 6-membered heteroaryl, wherein the heteroaryl contains 1˜4 heteroatoms independently selected from N, O, and S.

In some embodiments, Ring A is monocyclic 5-membered heteroaryl optionally substituted by 1-3 R⁰ groups, wherein the heteroaryl contains 1˜4 heteroatoms independently selected from N, O, and S. In some embodiments, Ring A is monocyclic 5-membered heteroaryl optionally substituted by 1-3 R⁰ groups, wherein the heteroaryl contains 1-2 heteroatoms independently selected from N and O. In some embodiments, Ring A is monocyclic 6-membered heteroaryl optionally substituted by 1-3 R⁰ groups, wherein the heteroaryl contains 1-4 heteroatoms independently selected from N, O, and S. In some embodiments, Ring A is monocyclic 6-membered heteroaryl optionally substituted by 1-3 R⁰ groups, wherein the heteroaryl contains 1-3 heteroatoms independently selected from N and O. In some embodiments, Ring A is a monocyclic 6-membered heteroaryl optionally substituted by 1-3 R⁰ groups, wherein the heteroaryl contains 1-2 heteroatoms independently selected from N and O. In some embodiments, Ring A is monocyclic 6-membered heteroaryl optionally substituted by 1-3 R⁰ groups, wherein the heteroaryl contains 1-3 nitrogen atoms. In some embodiments, Ring A is monocyclic 6-membered heteroaryl optionally substituted by 1-3 R⁰ groups, wherein the heteroaryl contains 1-2 nitrogen atoms. In some embodiments, Ring A is monocyclic 6-membered heteroaryl optionally substituted by 1-3 R⁰ groups, wherein the heteroaryl contains 1 nitrogen atom. In some embodiments, Ring A is monocyclic 6-membered heteroaryl optionally substituted by 1-2 R⁰ groups, wherein the heteroaryl contains 1 nitrogen atom. In some embodiments, Ring A is monocyclic 6-membered heteroaryl optionally substituted by 1 R⁰ group, wherein the heteroaryl contains 1 nitrogen atom. In some embodiments, Ring A is monocyclic 6-membered heteroaryl optionally substituted by 2 R⁰ groups, wherein the heteroaryl contains 1 nitrogen atom. In some embodiments, Ring A is monocyclic 6-membered heteroaryl optionally substituted by 1-3 R⁰ groups, wherein the heteroaryl contains 2 nitrogen atoms. In some embodiments, Ring A is monocyclic 6-membered heteroaryl optionally substituted by 1-2 R⁰ groups, wherein the heteroaryl contains 2 nitrogen atoms. In some embodiments, Ring A is monocyclic 6-membered heteroaryl optionally substituted by 1 R⁰ group, wherein the heteroaryl contains 2 nitrogen atoms. In some embodiments, Ring A is monocyclic 6-membered heteroaryl optionally substituted by 2 R⁰ groups, wherein the heteroaryl contains 2 nitrogen atoms. In some embodiments, Ring A is pyridinyl, pyrazinyl, pyrimidinyl, pyridazinyl, or triazinyl, each of which is optionally substituted by 1-3 R⁰ groups. In some embodiments, Ring A is pyridinyl or pyrimidinyl, each of which is optionally substituted by 1-3 R⁰ groups. In some embodiments, Ring A is pyridinyl optionally substituted by 1-3 R⁰ groups. In some embodiments, Ring A is pyrimidinyl optionally substituted by 1-3 R⁰ groups.

In some embodiments, Ring A is

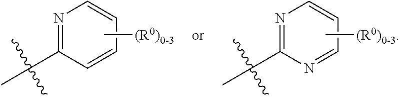

In some embodiments, Ring A fused bicyclic 9- to 10-membered heteroaryl or heterocyclyl, wherein the heteroaryl and heterocyclyl contain 1-4 heteroatoms independently selected from N, O, and S, each of which is optionally substituted by 1-3 R⁰ groups. In some embodiments, Ring A fused bicyclic 9- to 10-membered heteroaryl or heterocyclyl, wherein the heteroaryl and heterocyclyl contain 2-4 heteroatoms independently selected from N, O, and S, each of which is optionally substituted by 1-3 R⁰ groups. In some embodiments, Ring A is a fused bicyclic 9-membered heteroaryl or heterocyclyl containing 2-4 heteroatoms independently selected from N, O, and S, and is optionally substituted by 1-3 R⁰ groups. In some embodiments, Ring A is a fused bicyclic 9-membered heteroaryl or heterocyclyl containing 2-4 heteroatoms independently selected from N, O, and S, and is optionally substituted by 1 R⁰ group. In some embodiments, Ring A is a fused bicyclic 9-membered heteroaryl or heterocyclyl containing 2-4 heteroatoms independently selected from N, O, and S, and is optionally substituted by 2 R⁰ groups. In some embodiments, Ring A is a fused bicyclic 9-membered heteroaryl or heterocyclyl containing 2-4 heteroatoms independently selected from N, O, and S, and is optionally substituted by 3 R⁰ groups. In some embodiments, Ring A is a fused bicyclic 9-membered heteroaryl or heterocyclyl containing 2-4 heteroatoms independently selected from N, O, and S, and is optionally substituted by 4 R⁰ groups. In some embodiments, Ring A is an unsubstituted fused bicyclic 9-membered heteroaryl or heterocyclyl containing 2-4 heteroatoms independently selected from N, O, and S. In some embodiments, Ring A is a fused bicyclic 9-membered heteroaryl containing 2-4 heteroatoms independently selected from N, O, and S, and is optionally substituted by 1-3 R⁰ groups. In some embodiments, Ring A is a fused bicyclic 9-membered heterocyclyl containing 2-4 heteroatoms independently selected from N, O, and S, and is optionally substituted by 1-3 R⁰ groups.

In some embodiments, Ring A is:

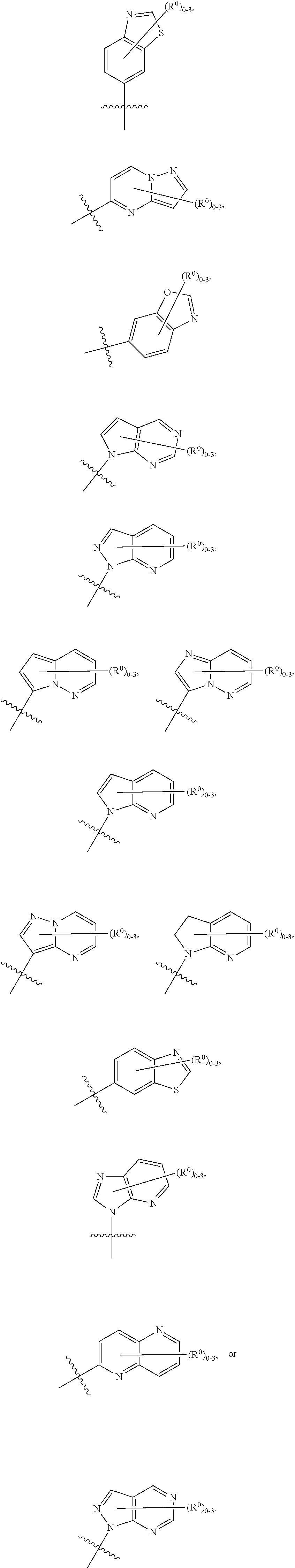

It is understood that when —(R⁰)₀₋₃ is drawn through a fused bicyclic heteroaryl or heterocyclyl, then one or both of the fused rings may be substituted by R⁰ groups. In some embodiments, only one ring of the fused bicyclic ring is substituted by R⁰ groups. In some embodiments, both rings of the fused bicyclic ring are substituted by R⁰ groups, such that the total number of R⁰ groups substituting the bicyclic ring is 0-3.

In some embodiments, each R⁰ is independently selected from halo, —CN, —NH₂, —NH(C₁-C₆ alkyl), C₁-C₆ alkyl, C₃-C₆ cycloalkyl, -(6- to 10-membered bridged heterocyclylene)-, and C₁-C₆ alkoxy, wherein the heterocyclylene contains 1-3 heteroatoms selected from N and O, or two R⁰ groups are taken together to form an oxo group. In some embodiments, each R⁰ is independently selected from halo, —CN, —NH₂, —NH(C₁-C₃ alkyl), C₁-C₃ alkyl, C₃-C₅ cycloalkyl, -(6- to 8-membered bridged heterocyclylene)-, and C₁-C₃ alkoxy, wherein the heterocyclylene contains 1-3 heteroatoms selected from N and O, or two R⁰ groups are taken together to form an oxo group.

In some embodiments, R⁰ is halo. In some embodiments, R⁰ is Cl, F, or Br. In some embodiments, R⁰ is C₁. In some embodiments, R⁰ is F. In some embodiments, R⁰ is Br.

In some embodiments, R⁰ is —CN.

In some embodiments, R⁰ is —NH₂.

In some embodiments, R⁰ is —NH(C₁-C₆ alkyl). In some embodiments, R⁰ is —NH(C₁-C₃ alkyl). In some embodiments, R⁰ is —NH(CH₃), —NH(CH₂CH₃), —NH(CH₂CH₂CH₃), or —NH(CH(CH₃)₂). In some embodiments, R⁰ is —NH(CH₃).

In some embodiments, R⁰ is C₁-C₆ alkyl. In some embodiments, R⁰ is C₁-C₃ alkyl. In some embodiments, R⁰ is methyl, ethyl, n-propyl, or isopropyl. In some embodiments, R⁰ is methyl. In some embodiments, R⁰ is ethyl. In some embodiments, R⁰ is n-propyl. In some embodiments, R⁰ is isopropyl.

In some embodiments, R⁰ is C₃-C₆ cycloalkyl. In some embodiments, R⁰ is C₃-C₅ cycloalkyl. In some embodiments, R⁰ is cyclopropyl, cyclobutyl, cyclopentyl, or cyclohexyl. In some embodiments, R⁰ is cyclopropyl.

In some embodiments, R⁰ is C₁-C₆ alkoxy. In some embodiments, R⁰ is C₁-C₃ alkoxy. In some embodiments, R⁰ is —OCH₃, —OCH₂CH₃, —OCH₂CH₂CH₃, or —OCH(CH₃)₂. In some embodiments, R⁰ is —OCH₃. In some embodiments, R⁰ is —OCH₂CH₃.

In some embodiments, R⁰ is -(6- to 8-membered bridged heterocyclylene)-, wherein the heterocyclylene contains 1-3 heteroatoms selected from N and O. In some embodiments, R⁰ is -(6- to 7-membered bridged heterocyclylene)-, wherein the heterocyclylene contains 1-3 heteroatoms selected from N and O. In some embodiments, R⁰ is -(6-membered bridged heterocyclylene)-, wherein the heterocyclylene contains 1-2 heteroatoms selected from N and O. In some embodiments, R⁰ is -(7-membered bridged heterocyclylene)-, wherein the heterocyclylene contains 1-2 heteroatoms selected from N and O. In some embodiments, R⁰ is -(8-membered bridged heterocyclylene)-, wherein the heterocyclylene contains 1-2 heteroatoms selected from N and O. In some embodiments, R⁰ is

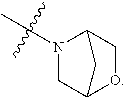 [SMILES: *N1CC2CC1CO2]

In some embodiments, two R⁰ groups are taken together to form an oxo group.

In some embodiments, Ring A is phenyl optionally substituted by 1-3 R⁰ groups; and each R⁰ is independently selected from halo, —CN, —NH₂, —NH(C₁-C₃ alkyl), C₁-C₃ alkyl, C₃-C₆ cycloalkyl, and C₁-C₃ alkoxy.

In some embodiments, Ring A is

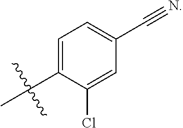 [SMILES: *c1ccc(C#N)cc1Cl]

In some embodiments, Ring A is a monocyclic 6-membered heteroaryl containing 1-2 heteroatoms independently selected from N and O and is optionally substituted by 1-3 R⁰ groups; and each R⁰ is independently selected from halo, —CN, —NH₂, —NH(C₁-C₆ alkyl), C₁-C₆ alkyl, C₃-C₆ cycloalkyl, and C₁-C₆ alkoxy.

In some embodiments, Ring A is:

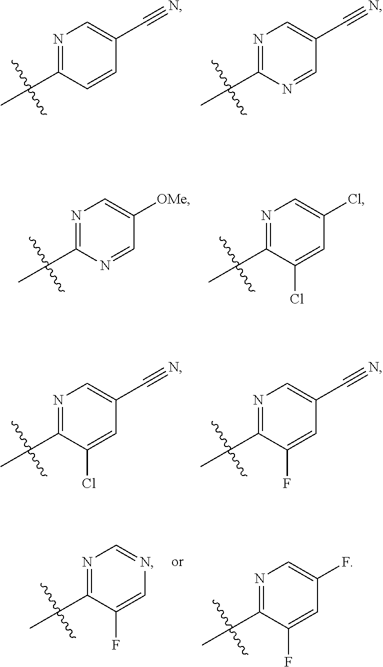

In some embodiments, Ring A is a fused bicyclic 9-membered heteroaryl or heterocyclyl containing 2-4 heteroatoms independently selected from N, O, and S, and is optionally substituted by 1-3 R⁰ groups; and each R⁰ is independently selected from halo, —CN, —NH₂, —NH(C₁-C₃ alkyl), C₁-C₃ alkyl, C₃-C₆ cycloalkyl, -(6- to 8-membered bridged heterocyclylene)-, and C₁-C₃ alkoxy, wherein the heterocyclylene contains 1-3 heteroatoms selected from N and O, or two R⁰ groups are taken together to form an oxo group.

In some embodiments, Ring A is:

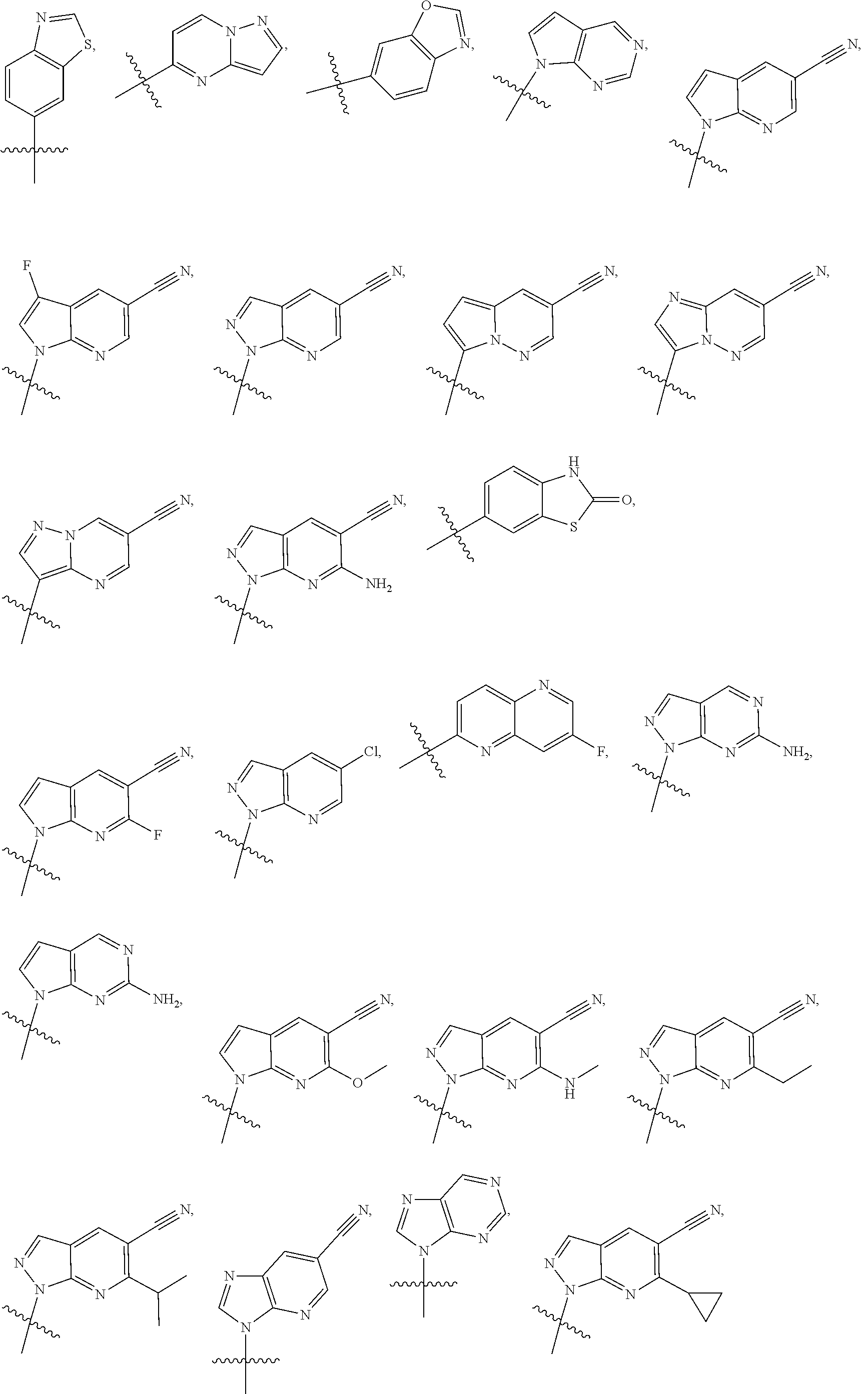

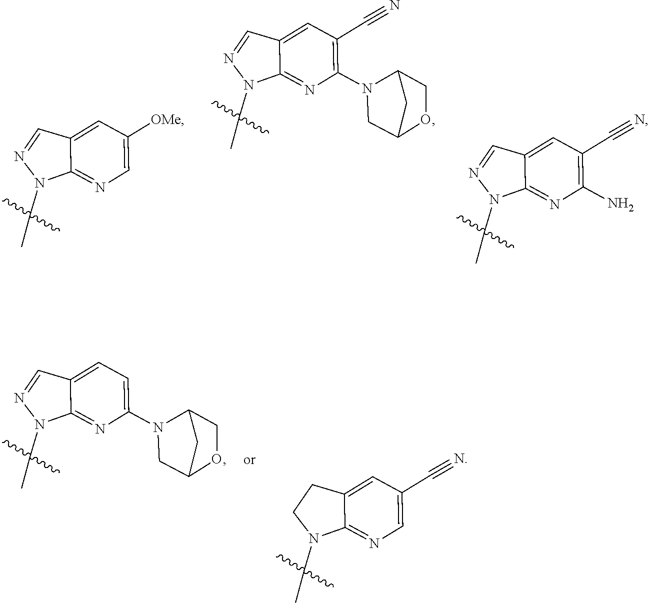 [SMILES: *n1ncc2cc(OC)cnc21 ; *n1ncc2cc(C#N)c(N3CC4CC3CO4)nc21 ; *n1ncc2cc(C#N)c(N)nc21 ; *n1ncc2ccc(N3CC4CC3CO4)nc21 ; *N1CCc2cc(C#N)cnc21]

In some embodiments, L¹ is —NH— or a bond. In some embodiments, L¹ is —NH—. In some embodiments, L¹ is a bond.

In some embodiments, L² is —NHC(O)—, —C(O)NH—, —SO₂NH—, —NHSO₂—, or —(C₁-C₆ alkylene)_{z}(5-membered heteroarylene)-, wherein the heteroarylene contains 1-3 heteroatoms selected from N, O, and S. In some embodiments, L² is 5-membered heteroarylene containing 1-3 heteroatoms selected from N and O. In some embodiments, L² is —NHC(O)— or triazolylene.

In some embodiments, L² is —NHC(O)—. In some embodiments, L² is —C(O)NH—. In some embodiments, L² is —SO₂NH—. In some embodiments, L² is —NHSO₂—.

In some embodiments, L² is 5-membered heteroarylene containing 1-3 heteroatoms selected from N, O, and S. In some embodiments, L² is 5-membered heteroarylene containing 1-3 heteroatoms selected from N and O. In some embodiments, L² is 5-membered heteroarylene containing 2 nitrogen atoms. In some embodiments, L² is 5-membered heteroarylene containing 3 nitrogen atoms. In some embodiments, L² is 5-membered heteroarylene containing 1 nitrogen atom and 1 oxygen atom. In some embodiments, L² is 5-membered heteroarylene containing 1 nitrogen atom and 1 sulfur atom. In some embodiments, L² is 5-membered heteroarylene containing 1 nitrogen atom. In some embodiments, L² is triazolylene, imidazolylene, pyrazolylene, oxazolylene, isoxazolylene, or pyrrolylene.

In some embodiments, L² is —(C₁-C₆ alkylene)_{z}(5-membered heteroarylene)-, wherein the heteroarylene contains 1-3 heteroatoms selected from N, O, and S. In some embodiments, L² is —(C₁-C₃ alkylene)_{z}(5-membered heteroarylene)-, wherein the heteroarylene contains 1-3 heteroatoms selected from N, O, and S. In some embodiments, L² is —(C₁-C₃ alkylene)_{z}(5-membered heteroarylene)-, wherein the heteroarylene contains 1-3 heteroatoms selected from N and O. In some embodiments, the 5-membered heteroarylene contains 1-3 nitrogen atoms. In some embodiments, the 5-membered heteroarylene contains 1 nitrogen atom. In some embodiments, the 5-membered heteroarylene contains 2 nitrogen atoms. In some embodiments, the 5-membered heteroarylene contains 3 nitrogen atoms. In some embodiments, the 5-membered heteroarylene contains 1 nitrogen atom and 1 oxygen atom. In some embodiments, the 5-membered heteroarylene contains 1 nitrogen atom and 1 sulfur atom. In some embodiments, the 5-membered heteroarylene is triazolylene, imidazolylene, pyrazolylene, oxazolylene, isoxazolylene, or pyrrolylene. In some embodiments, z is 0. In some embodiments, z is 1. In some embodiments, —(C₁-C₆ alkylene)_{z}- is —CH₂—. In some embodiments, —(C₁-C₆ alkylene)_{z}- is —CH₂CH₂—. In some embodiments, —(C₁-C₆ alkylene)_{z}- is —CH₂CH₂CH₂—. In some embodiments, L² is

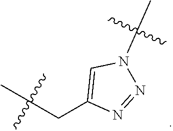 [SMILES: *Cc1cn(*)nn1]

In some embodiments, L² is

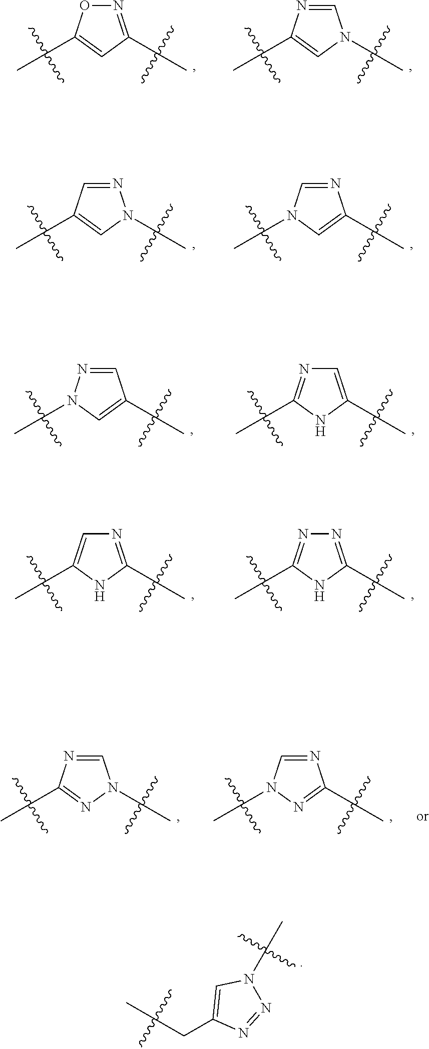

In some embodiments, L² is

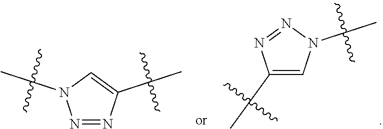 [SMILES: *c1cn(*)nn1 ; *c1cn(*)nn1]

In some embodiments, L³ is —NR⁹ (C₁-C₆ alkylene)NR⁹—, —NR⁹C(O)(C₁-C₆ alkylene)_{z}(4- to 7-membered heterocyclylene)-, -(4- to 7-membered heterocyclylene)CR¹¹R¹²—, -(4- to 7-membered heterocyclylene)(CO)_{z}—, -(4- to 7-membered heterocyclylene)(NR⁹)_{z}—, —(NR⁹)_{z}(4- to 7-membered heterocyclylene)(C₁-C₆ alkylene)_{z}-, —NR⁹ (C₁-C₆ alkylene)_{z}(4- to 7-membered heterocyclylene)-, —NR⁹C(O)(phenylene)NR⁹—, —(C₁-C₆ alkylene)_{z}(4- to 7-membered heterocyclylene)(C₁-C₆ alkylene)_{z}-, —O(C₁-C₆ alkylene)_{z}(4- to 7-membered heterocyclylene)(C₁-C₆ alkylene)_{z}-, -(6- to 10-membered bridged heterocyclylene)(C₁-C₆ alkylene)_{z}-, -(7- to 10-membered fused bicyclic heterocyclylene)(C₁-C₆ alkylene)_{z}-, or —(O)_{z}(6- to 10-membered spiro heterocyclylene)(C₁-C₆ alkylene)_{z}-, wherein the heterocyclylene contains 1-3 heteroatoms selected from N and O and is optionally substituted by 1-5 R⁷ groups.

In some embodiments, L³ is —NR⁹ (C₁-C₃ alkylene)NR⁹—, —NR⁹C(O)(C₁-C₃ alkylene)_{z}(4- to 7-membered heterocyclylene)-, -(4- to 7-membered heterocyclylene)CR¹¹R¹²—, -(4- to 7-membered heterocyclylene)(CO)_{z}—, -(4- to 7-membered heterocyclylene)(NR⁹)_{z}—, —(NR⁹)_{z}(4- to 7-membered heterocyclylene)(C₁-C₃ alkylene)_{z}-, —NR⁹ (C₁-C₃ alkylene)_{z}(4- to 7-membered heterocyclylene)-, —NR⁹C(O)(phenylene)NR⁹—, —(C₁-C₃ alkylene)_{z}(4- to 7-membered heterocyclylene)(C₁-C₃ alkylene)_{z}-, —O(C₁-C₃ alkylene)_{z}(4- to 7-membered heterocyclylene)(C₁-C₃ alkylene)_{z}-, -(6- to 10-membered bridged heterocyclylene)(C₁-C₃ alkylene)_{z}-, -(7- to 10-membered fused bicyclic heterocyclylene)(C₁-C₆ alkylene)_{z}-, or —(O)_{z}(6- to 10-membered spiro heterocyclylene)(C₁-C₃ alkylene)_{z}-, wherein the heterocyclylene contains 1-3 heteroatoms selected from N and O and is optionally substituted by 1 or 2 R⁷ groups.

In some embodiments, L³ is —NR⁹ (C₁-C₆ alkylene)NR⁹—. In some embodiments, L³ is —NR⁹ (C₁-C₃ alkylene)NR⁹—. In some embodiments, L³ is —NR⁹ (CH₂)NR⁹—, —NR⁹ (CH₂CH₂)NR⁹—, or —NR⁹ (CH₂CH₂CH₂)NR⁹—. In some embodiments, L³ is —NR⁹ (CH₂CH₂CH₂)NR⁹—.

In some embodiments, L³ is —NR⁹C(O)(C₁-C₆ alkylene)_{z}(4- to 7-membered heterocyclylene)-, wherein the heterocyclylene contains 1-3 heteroatoms selected from N and O and is optionally substituted by 1-5 R⁷ groups. In some embodiments, L³ is —NR⁹C(O)(C₁-C₃ alkylene)_{z}(4- to 7-membered heterocyclylene)-, wherein the heterocyclylene contains 1-3 heteroatoms selected from N and O and is optionally substituted by 1-5 R⁷ groups. In some embodiments, L³ is —NR⁹C(O)(C₁-C₃ alkylene)_{z}(4- to 7-membered heterocyclylene)-, wherein the heterocyclylene contains 1-3 heteroatoms selected from N and O and is optionally substituted by 1-3 R⁷ groups. In some embodiments, L³ is —NR⁹C(O)(C₁-C₃ alkylene)_{z}(4- to 7-membered heterocyclylene)-, wherein the heterocyclylene contains 1-2 nitrogen atoms and is optionally substituted by 1-3 R⁷ groups. In some embodiments, L³ is —NR⁹C(O)(C₁-C₃ alkylene)_{z}(5- to 6-membered heterocyclylene)-, wherein the heterocyclylene contains 1-2 nitrogen atoms and is optionally substituted by 1-3 R⁷ groups. In some embodiments, L³ is —NR⁹C(O)(C₁-C₃ alkylene)_{z}(6-membered heterocyclylene)-, wherein the heterocyclylene contains 1-2 nitrogen atoms and is optionally substituted by 1-3 R⁷ groups. In some embodiments, L³ is —NR⁹C(O)(6-membered heterocyclylene)-, wherein the heterocyclylene contains 1-2 nitrogen atoms and is optionally substituted by 1-3 R⁷ groups. In some embodiments, L³ is —NR⁹C(O)CH₂(6-membered heterocyclylene)-, wherein the heterocyclylene contains 1-2 nitrogen atoms and is optionally substituted by 1-3 R⁷ groups.

In some embodiments, L³ is -(4- to 7-membered heterocyclylene)CR¹¹R¹²—, wherein the heterocyclylene contains 1-3 heteroatoms selected from N and O and is optionally substituted by 1-5 R⁷ groups. In some embodiments, L³ is -(4- to 7-membered heterocyclylene)CR¹¹R¹²—, wherein the heterocyclylene contains 1-2 heteroatoms selected from N and O and is optionally substituted by 1-5 R⁷ groups. In some embodiments, L³ is -(4- to 7-membered heterocyclylene)CR¹¹R¹²—, wherein the heterocyclylene contains 1-2 nitrogen atoms and is optionally substituted by 1-5 R⁷ groups. In some embodiments, L³ is -(5- to 6-membered heterocyclylene)CR¹¹R¹²—, wherein the heterocyclylene contains 1-2 nitrogen atoms and is optionally substituted by 1-5 R⁷ groups. In some embodiments, L³ is -(6-membered heterocyclylene)CR¹¹R¹²—, wherein the heterocyclylene contains 1-2 nitrogen atoms and is optionally substituted by 1-5 R⁷ groups.

In some embodiments, L³ is -(4- to 7-membered heterocyclylene)(CO)_{z}—, wherein the heterocyclylene contains 1-3 heteroatoms selected from N and O and is optionally substituted by 1-5 R⁷ groups. In some embodiments, L³ is -(5- to 7-membered heterocyclylene)(CO)_{z}—, wherein the heterocyclylene contains 1-2 heteroatoms selected from N and O and is optionally substituted by 1-5 R⁷ groups. In some embodiments, L³ is -(5- to 7-membered heterocyclylene)(CO)_{z}—, wherein the heterocyclylene contains 1-2 nitrogen atoms and is optionally substituted by 1-5 R⁷ groups. In some embodiments, L³ is -(6- to 7-membered heterocyclylene)(CO)_{z}—, wherein the heterocyclylene contains 1-2 nitrogen atoms and is optionally substituted by 1-5 R⁷ groups. In some embodiments, L³ is -(6- to 7-membered heterocyclylene)-, wherein the heterocyclylene contains 1-2 nitrogen atoms and is optionally substituted by 1-5 R⁷ groups. In some embodiments, L³ is -(6- to 7-membered heterocyclylene)(CO)—, wherein the heterocyclylene contains 1-2 nitrogen atoms and is optionally substituted by 1-5 R⁷ groups.

In some embodiments, L³ is -(4- to 7-membered heterocyclylene)(NR⁹)_{z}—, wherein the heterocyclylene contains 1-3 heteroatoms selected from N and O and is optionally substituted by 1-5 R⁷ groups. In some embodiments, L³ is -(4- to 7-membered heterocyclylene)(NR⁹)_{z}—, wherein the heterocyclylene contains 1-2 heteroatoms selected from N and O and is optionally substituted by 1-5 R⁷ groups. In some embodiments, L³ is -(4- to 7-membered heterocyclylene)(NR⁹)_{z}—, wherein the heterocyclylene contains 1-2 nitrogen atoms and is optionally substituted by 1-5 R⁷ groups. In some embodiments, L³ is -(5- to 7-membered heterocyclylene)(NR⁹)_{z}—, wherein the heterocyclylene contains 1-2 nitrogen atoms and is optionally substituted by 1-5 R⁷ groups. In some embodiments, L³ is -(6- to 7-membered heterocyclylene)(NR⁹)_{z}—, wherein the heterocyclylene contains 1-2 nitrogen atoms and is optionally substituted by 1-5 R⁷ groups. In some embodiments, L³ is -(6- to 7-membered heterocyclylene)-, wherein the heterocyclylene contains 1-2 nitrogen atoms and is optionally substituted by 1-5 R⁷ groups. In some embodiments, L³ is -(6- to 7-membered heterocyclylene)(NR⁹)—, wherein the heterocyclylene contains 1-2 nitrogen atoms and is optionally substituted by 1-5 R⁷ groups.

In some embodiments, L³ is —(NR⁹)_{z}(4- to 7-membered heterocyclylene)(C₁-C₆ alkylene)_{z}-, wherein the heterocyclylene contains 1-3 heteroatoms selected from N and O and is optionally substituted by 1-5 R⁷ groups. In some embodiments, L³ is —(NR⁹)_{z}(4- to 7-membered heterocyclylene)(C₁-C₆ alkylene)_{z}-, wherein the heterocyclylene contains 1-2 heteroatoms selected from N and O and is optionally substituted by 1-5 R⁷ groups. In some embodiments, L³ is —(NR⁹)_{z}(4- to 7-membered heterocyclylene)(C₁-C₆ alkylene)_{z}-, wherein the heterocyclylene contains 1-2 nitrogen atoms and is optionally substituted by 1-5 R⁷ groups. In some embodiments, L³ is —(NR⁹)_{z}(5- to 7-membered heterocyclylene)(C₁-C₆ alkylene)_{z}-, wherein the heterocyclylene contains 1-2 nitrogen atoms and is optionally substituted by 1-5 R⁷ groups. In some embodiments, L³ is —(NR⁹)_{z}(6- to 7-membered heterocyclylene)(C₁-C₆ alkylene)_{z}-, wherein the heterocyclylene contains 1-2 nitrogen atoms and is optionally substituted by 1-5 R⁷ groups. In some embodiments, L³ is —(NR⁹)_{z}(6- to 7-membered heterocyclylene)(C₁-C₃ alkylene)_{z}-, wherein the heterocyclylene contains 1-2 nitrogen atoms and is optionally substituted by 1-5 R⁷ groups. In some embodiments, L³ is —(NR⁹)_{z}(6- to 7-membered heterocyclylene)(CH₂)_{z}—, wherein the heterocyclylene contains 1-2 nitrogen atoms and is optionally substituted by 1-5 R⁷ groups. In some embodiments, L³ is —(NR⁹)_{z}(6- to 7-membered heterocyclylene)(CH₂)—, wherein the heterocyclylene contains 1-2 nitrogen atoms and is optionally substituted by 1-5 R⁷ groups. In some embodiments, L³ is —(NR⁹)_{z}(6- to 7-membered heterocyclylene)-, wherein the heterocyclylene contains 1-2 nitrogen atoms and is optionally substituted by 1-5 R⁷ groups. In some embodiments, L³ is -(6- to 7-membered heterocyclylene)(CH₂)—, wherein the heterocyclylene contains 1-2 nitrogen atoms and is optionally substituted by 1-5 R⁷ groups. In some embodiments, L³ is -(6- to 7-membered heterocyclylene)-, wherein the heterocyclylene contains 1-2 nitrogen atoms and is optionally substituted by 1-5 R⁷ groups.

In some embodiments, L³ is —NR⁹ (C₁-C₆ alkylene)_{z}(4- to 7-membered heterocyclylene)-, wherein the heterocyclylene contains 1-3 heteroatoms selected from N and O and is optionally substituted by 1-5 R⁷ groups. In some embodiments, L³ is —NR⁹ (C₁-C₆ alkylene)_{z}(4- to 7-membered heterocyclylene)-, wherein the heterocyclylene contains 1-2 heteroatoms selected from N and O and is optionally substituted by 1-5 R⁷ groups. In some embodiments, L³ is —NR⁹ (C₁-C₆ alkylene)_{z}(4- to 7-membered heterocyclylene)-, wherein the heterocyclylene contains 1-2 nitrogen atoms and is optionally substituted by 1-5 R⁷ groups. In some embodiments, L³ is —NR⁹ (C₁-C₆ alkylene)_{z}(5- to 7-membered heterocyclylene)-, wherein the heterocyclylene contains 1-2 nitrogen atoms and is optionally substituted by 1-5 R⁷ groups. In some embodiments, L³ is —NR⁹ (C₁-C₆ alkylene)_{z}(6- to 7-membered heterocyclylene)-, wherein the heterocyclylene contains 1-2 nitrogen atoms and is optionally substituted by 1-5 R⁷ groups. In some embodiments, L³ is —NR⁹ (C₁-C₆ alkylene)_{z}(6-membered heterocyclylene)-, wherein the heterocyclylene contains 1-2 nitrogen atoms and is optionally substituted by 1-5 R⁷ groups. In some embodiments, L³ is —NR⁹ (C₁-C₃ alkylene)_{z}(6-membered heterocyclylene)-, wherein the heterocyclylene contains 1-2 nitrogen atoms and is optionally substituted by 1-5 R⁷ groups. In some embodiments, L³ is —NR⁹ (CH₂)_{z}(6-membered heterocyclylene)-, wherein the heterocyclylene contains 1-2 nitrogen atoms and is optionally substituted by 1-5 R⁷ groups. In some embodiments, L³ is —NR⁹ (CH₂)(6-membered heterocyclylene)-, wherein the heterocyclylene contains 1-2 nitrogen atoms and is optionally substituted by 1-5 R⁷ groups. In some embodiments, L³ is —NR⁹ (6-membered heterocyclylene)-, wherein the heterocyclylene contains 1-2 nitrogen atoms and is optionally substituted by 1-5 R⁷ groups.

In some embodiments, L³ is —NR⁹C(O)(phenylene)NR⁹—.

In some embodiments, L³ is —(C₁-C₆ alkylene)_{z}(4- to 7-membered heterocyclylene)-, wherein the heterocyclylene contains 1-3 heteroatoms selected from N and O and is optionally substituted by 1-5 R⁷ groups. In some embodiments, L³ is —(C₁-C₆ alkylene)_{z}(4- to 7-membered heterocyclylene)-, wherein the heterocyclylene contains 1-2 heteroatoms selected from N and O and is optionally substituted by 1-5 R⁷ groups. In some embodiments, L³ is —(C₁-C₆ alkylene)_{z}(5- to 7-membered heterocyclylene)-, wherein the heterocyclylene contains 1-2 heteroatoms selected from N and O and is optionally substituted by 1-5 R⁷ groups. In some embodiments, L³ is —(C₁-C₆ alkylene)_{z}(5- to 7-membered heterocyclylene)-, wherein the heterocyclylene contains 1-2 nitrogen atoms and is optionally substituted by 1-5 R⁷ groups. In some embodiments, L³ is —(C₁-C₃ alkylene)_{z}(5- to 7-membered heterocyclylene)-, wherein the heterocyclylene contains 1-2 nitrogen atoms and is optionally substituted by 1-5 R⁷ groups. In some embodiments, L³ is —(C₁-C₃ alkylene)_{z}(6-membered heterocyclylene)-, wherein the heterocyclylene contains 1-2 nitrogen atoms and is optionally substituted by 1-5 R⁷ groups. In some embodiments, L³ is —(CH₂)(6-membered heterocyclylene)-, wherein the heterocyclylene contains 1-2 nitrogen atoms and is optionally substituted by 1-5 R⁷ groups. In some embodiments, L³ is —(CH₂CH₂)(6-membered heterocyclylene)-, wherein the heterocyclylene contains 1-2 nitrogen atoms and is optionally substituted by 1-5 R⁷ groups. In some embodiments, L³ is -(6-membered heterocyclylene)-, wherein the heterocyclylene contains 1-2 nitrogen atoms and is optionally substituted by 1-5 R⁷ groups.

In some embodiments, L³ is —(C₁-C₆ alkylene)_{z}(4- to 7-membered heterocyclylene)(C₁-C₆ alkylene)_{z}-, wherein the heterocyclylene contains 1-3 heteroatoms selected from N and O and is optionally substituted by 1-5 R⁷ groups. In some embodiments, L³ is —(C₁-C₆ alkylene)_{z}(4- to 7-membered heterocyclylene)(C₁-C₆ alkylene)_{z}-, wherein the heterocyclylene contains 1-2 heteroatoms selected from N and O and is optionally substituted by 1-5 R⁷ groups. In some embodiments, L³ is —(C₁-C₃ alkylene)_{z}(5- to 7-membered heterocyclylene)(C₁-C₃ alkylene)_{z}-, wherein the heterocyclylene contains 1-2 heteroatoms selected from N and O and is optionally substituted by 1-5 R⁷ groups. In some embodiments, L³ is —(C₁-C₃ alkylene)_{z}(5- to 7-membered heterocyclylene)(C₁-C₃ alkylene)_{z}-, wherein the heterocyclylene contains 1-2 nitrogen atoms and is optionally substituted by 1-5 R⁷ groups. In some embodiments, L³ is —(C₁-C₃ alkylene)_{z}(6-membered heterocyclylene)(C₁-C₃ alkylene)_{z}-, wherein the heterocyclylene contains 1-2 nitrogen atoms and is optionally substituted by 1-5 R⁷ groups. In some embodiments, L³ is —(CH₂)(6-membered heterocyclylene)CH₂—, wherein the heterocyclylene contains 1-2 nitrogen atoms and is optionally substituted by 1-5 R⁷ groups. In some embodiments, L³ is -(5- to 6-membered heterocyclylene)CH₂—, wherein the heterocyclylene contains 1-2 nitrogen atoms and is optionally substituted by 1-5 R⁷ groups. In some embodiments, L³ is -(7-membered heterocyclylene)CH₂—, wherein the heterocyclylene contains 1-2 nitrogen atoms and is optionally substituted by 1-5 R⁷ groups.

In some embodiments, L³ is —O(C₁-C₆ alkylene)_{z}(4- to 7-membered heterocyclylene)-, wherein the heterocyclylene contains 1-3 heteroatoms selected from N and O and is optionally substituted by 1-5 R⁷ groups. In some embodiments, L³ is —O(C₁-C₆ alkylene)_{z}(4- to 7-membered heterocyclylene)-, wherein the heterocyclylene contains 1-2 heteroatoms selected from N and O and is optionally substituted by 1-5 R⁷ groups. In some embodiments, L³ is —O(C₁-C₆ alkylene)_{z}(4- to 7-membered heterocyclylene)-, wherein the heterocyclylene contains 1-2 nitrogen atoms and is optionally substituted by 1-5 R⁷ groups. In some embodiments, L³ is —O(C₁-C₃ alkylene)_{z}(4- to 7-membered heterocyclylene)-, wherein the heterocyclylene contains 1-2 nitrogen atoms and is optionally substituted by 1-5 R⁷ groups. In some embodiments, L³ is —O(C₁-C₃ alkylene)(4- to 7-membered heterocyclylene)-, wherein the heterocyclylene contains 1-2 nitrogen atoms and is optionally substituted by 1-5 R⁷ groups. In some embodiments, L³ is —O(CH₂)(4- to 7-membered heterocyclylene)-, wherein the heterocyclylene contains 1-2 nitrogen atoms and is optionally substituted by 1-5 R⁷ groups. In some embodiments, L³ is —O(4- to 7-membered heterocyclylene)-, wherein the heterocyclylene contains 1-2 nitrogen atoms and is optionally substituted by 1-5 R⁷ groups. In some embodiments, L³ is —O(4- to 6-membered heterocyclylene)-, wherein the heterocyclylene contains 1-2 nitrogen atoms and is optionally substituted by 1-5 R⁷ groups.

In some embodiments, L³ is —O(C₁-C₆ alkylene)_{z}(4- to 7-membered heterocyclylene)(C₁-C₆ alkylene)_{z}-, wherein the heterocyclylene contains 1-3 heteroatoms selected from N and O and is optionally substituted by 1-5 R⁷ groups. In some embodiments, L³ is —O(C₁-C₆ alkylene)_{z}(4- to 7-membered heterocyclylene)(C₁-C₆ alkylene)_{z}-, wherein the heterocyclylene contains 1-2 heteroatoms selected from N and O and is optionally substituted by 1-5 R⁷ groups. In some embodiments, L³ is —O(C₁-C₆ alkylene)_{z}(4- to 7-membered heterocyclylene)(C₁-C₆ alkylene)_{z}-, wherein the heterocyclylene contains 1-2 nitrogen atoms and is optionally substituted by 1-5 R⁷ groups. In some embodiments, L³ is —O(C₁-C₃ alkylene)_{z}(4- to 7-membered heterocyclylene)(C₁-C₃ alkylene)_{z}-, wherein the heterocyclylene contains 1-2 nitrogen atoms and is optionally substituted by 1-5 R⁷ groups. In some embodiments, L³ is —O(C₁-C₃ alkylene)(4- to 7-membered heterocyclylene)(C₁-C₃ alkylene)-, wherein the heterocyclylene contains 1-2 nitrogen atoms and is optionally substituted by 1-5 R⁷ groups. In some embodiments, L³ is —O(4- to 7-membered heterocyclylene)(C₁-C₃ alkylene)-, wherein the heterocyclylene contains 1-2 nitrogen atoms and is optionally substituted by 1-5 R⁷ groups. In some embodiments, L³ is —O(CH₂)(4- to 7-membered heterocyclylene)(CH₂)—, wherein the heterocyclylene contains 1-2 nitrogen atoms and is optionally substituted by 1-5 R⁷ groups. In some embodiments, L³ is —O(4- to 7-membered heterocyclylene)(CH₂)—, wherein the heterocyclylene contains 1-2 nitrogen atoms and is optionally substituted by 1-5 R⁷ groups. In some embodiments, L³ is —O(4- to 6-membered heterocyclylene)CH₂—, wherein the heterocyclylene contains 1-2 nitrogen atoms and is optionally substituted by 1-5 R⁷ groups. In some embodiments, the heterocyclyene is saturated. In some embodiments, the heterocyclylene is partially unsaturated.

In some embodiments, L³ is -(6- to 10-membered bridged heterocyclylene)(C₁-C₆ alkylene)_{z}-, wherein the heterocyclylene contains 1-3 heteroatoms selected from N and O and is optionally substituted by 1-5 R⁷ groups. In some embodiments, L³ is -(6- to 10-membered bridged heterocyclylene)(C₁-C₆ alkylene)_{z}-, wherein the heterocyclylene contains 1-2 heteroatoms selected from N and O and is optionally substituted by 1-5 R⁷ groups. In some embodiments, L³ is -(7- to 9-membered bridged heterocyclylene)(C₁-C₆ alkylene)_{z}-, wherein the heterocyclylene contains 1-2 heteroatoms selected from N and O and is optionally substituted by 1-5 R⁷ groups. In some embodiments, L³ is -(7- to 9-membered bridged heterocyclylene)(C₁-C₃ alkylene)_{z}-, wherein the heterocyclylene contains 1-2 heteroatoms selected from N and O and is optionally substituted by 1-5 R⁷ groups. In some embodiments, L³ is -(7- to 9-membered bridged heterocyclylene)(CH₂)—, wherein the heterocyclylene contains 1-2 heteroatoms selected from N and O and is optionally substituted by 1-5 R⁷ groups. In some embodiments, L³ is -(7- to 9-membered bridged heterocyclylene)-, wherein the heterocyclylene contains 1-2 heteroatoms selected from N and O and is optionally substituted by 1-5 R⁷ groups. In some embodiments, L³ is -(7-membered bridged heterocyclylene)(C₁-C₆ alkylene)_{z}-, wherein the heterocyclylene contains 1-2 heteroatoms selected from N and O and is optionally substituted by 1-5 R⁷ groups. In some embodiments, L³ is -(9-membered bridged heterocyclylene)(C₁-C₆ alkylene)_{z}-, wherein the heterocyclylene contains 1-2 heteroatoms selected from N and O and is optionally substituted by 1-5 R⁷ groups.

In some embodiments, L³ is -(7- to 10-membered fused bicyclic heterocyclylene)(C₁-C₆ alkylene)_{z}-, wherein the heterocyclylene contains 1-3 heteroatoms selected from N and O and is optionally substituted by 1-5 R⁷ groups. In some embodiments, L³ is -(7- to 10-membered fused bicyclic heterocyclylene)(C₁-C₃ alkylene)_{z}-, wherein the heterocyclylene contains 1-3 heteroatoms selected from N and O and is optionally substituted by 1-5 R⁷ groups. In some embodiments, L³ is -(7-membered fused bicyclic heterocyclylene)(C₁-C₆ alkylene)_{z}-, wherein the heterocyclylene contains 1-3 heteroatoms selected from N and O and is optionally substituted by 1-5 R⁷ groups. In some embodiments, L³ is -(7-membered fused bicyclic heterocyclylene)(C₁-C₃ alkylene)_{z}-, wherein the heterocyclylene contains 1-3 heteroatoms selected from N and O and is optionally substituted by 1-5 R⁷ groups. In some embodiments, L³ is -(7-membered fused bicyclic heterocyclylene)(C₁-C₃ alkylene)_{z}-, wherein the heterocyclylene contains 3 heteroatoms selected from N and O and is optionally substituted by 1-5 R⁷ groups. In some embodiments, L³ is -(7-membered fused bicyclic heterocyclylene)(CH₂)—, wherein the heterocyclylene contains 1-3 heteroatoms selected from N and O and is optionally substituted by 1-5 R⁷ groups. In some embodiments, L³ is -(7-membered fused bicyclic heterocyclylene)-, wherein the heterocyclylene contains 1-3 heteroatoms selected from N and O and is optionally substituted by 1-5 R⁷ groups. In some embodiments, L³ is -(8-membered fused bicyclic heterocyclylene)(C₁-C₆ alkylene)_{z}-, wherein the heterocyclylene contains 1-3 heteroatoms selected from N and O and is optionally substituted by 1-5 R⁷ groups. In some embodiments, L³ is -(8-membered fused bicyclic heterocyclylene)(C₁-C₃ alkylene)_{z}-, wherein the heterocyclylene contains 1-3 heteroatoms selected from N and O and is optionally substituted by 1-5 R⁷ groups. In some embodiments, L³ is -(8-membered fused bicyclic heterocyclylene)(C₁-C₃ alkylene)_{z}-, wherein the heterocyclylene contains 3 heteroatoms selected from N and O and is optionally substituted by 1-5 R⁷ groups. In some embodiments, L³ is -(8-membered fused bicyclic heterocyclylene)(CH₂)—, wherein the heterocyclylene contains 1-3 heteroatoms selected from N and O and is optionally substituted by 1-5 R⁷ groups. In some embodiments, L³ is -(8-membered fused bicyclic heterocyclylene)-, wherein the heterocyclylene contains 1-3 heteroatoms selected from N and O and is optionally substituted by 1-5 R⁷ groups. In some embodiments, L³ is -(9-membered fused bicyclic heterocyclylene)(C₁-C₆ alkylene)_{z}-, wherein the heterocyclylene contains 1-3 heteroatoms selected from N and O and is optionally substituted by 1-5 R⁷ groups. In some embodiments, L³ is -(9-membered fused bicyclic heterocyclylene)(C₁-C₃ alkylene)_{z}-, wherein the heterocyclylene contains 1-3 heteroatoms selected from N and O and is optionally substituted by 1-5 R⁷ groups. In some embodiments, L³ is -(9-membered fused bicyclic heterocyclylene)(C₁-C₃ alkylene)_{z}-, wherein the heterocyclylene contains 3 heteroatoms selected from N and O and is optionally substituted by 1-5 R⁷ groups. In some embodiments, L³ is -(9-membered fused bicyclic heterocyclylene)(CH₂)—, wherein the heterocyclylene contains 1-3 heteroatoms selected from N and O and is optionally substituted by 1-5 R⁷ groups. In some embodiments, L³ is -(9-membered fused bicyclic heterocyclylene)-, wherein the heterocyclylene contains 1-3 heteroatoms selected from N and O and is optionally substituted by 1-5 R⁷ groups.

In some embodiments, L³ is —(O)_{z}(6- to 10-membered spiro heterocyclylene)(C₁-C₆ alkylene)_{z}-, wherein the heterocyclylene contains 1-3 heteroatoms selected from N and O and is optionally substituted by 1-5 R⁷ groups. In some embodiments, L³ is —(O)_{z}(7- to 10-membered Spiro heterocyclylene)(C₁-C₆ alkylene)_{z}-, wherein the heterocyclylene contains 1-3 heteroatoms selected from N and O and is optionally substituted by 1-5 R⁷ groups. In some embodiments, L³ is —(O)_{z}(7- to 9-membered spiro heterocyclylene)(C₁-C₆ alkylene)_{z}-, wherein the heterocyclylene contains 1-3 heteroatoms selected from N and O and is optionally substituted by 1-5 R⁷ groups. In some embodiments, L³ is —O(7- to 9-membered spiro heterocyclylene)(C₁-C₆ alkylene)-, wherein the heterocyclylene contains 1-3 heteroatoms selected from N and O and is optionally substituted by 1-5 R⁷ groups. In some embodiments, L³ is —O(7- to 9-membered spiro heterocyclylene)(CH₂)—, wherein the heterocyclylene contains 1-3 heteroatoms selected from N and O and is optionally substituted by 1-5 R⁷ groups. In some embodiments, L³ is —O(6- to 10-membered spiro heterocyclylene)-, wherein the heterocyclylene contains 1-3 heteroatoms selected from N and O and is optionally substituted by 1-5 R⁷ groups. In some embodiments, L³ is -(6- to 10-membered spiro heterocyclylene)(C₁-C₆ alkylene)-, wherein the heterocyclylene contains 1-3 heteroatoms selected from N and O and is optionally substituted by 1-5 R⁷ groups. In some embodiments, L³ is -(6- to 10-membered spiro heterocyclylene)(CH₂)—, wherein the heterocyclylene contains 1-3 heteroatoms selected from N and O and is optionally substituted by 1-5 R⁷ groups. In some embodiments, L³ is -(6- to 10-membered spiro heterocyclylene)-, wherein the heterocyclylene contains 1-3 heteroatoms selected from N and O and is optionally substituted by 1-5 R⁷ groups.

In some embodiments, each R⁹ is independently H or C₁-C₆ alkyl. In some embodiments, each R⁹ is independently H or C₁-C₃ alkyl. In some embodiments, each R⁹ is independently H or —CH₃.

In some embodiments, R⁹ is H.

In some embodiments, R⁹ is C₁-C₆ alkyl. In some embodiments, R⁹ is C₁-C₃ alkyl. In some embodiments, R⁹ is methyl, ethyl, n-propyl, or isopropyl. In some embodiments, R⁹ is methyl. In some embodiments, R⁹ is ethyl. In some embodiments, R⁹ is n-propyl. In some embodiments, R⁹ is isopropyl.

In some embodiments, each z is independently 0 or 1. In some embodiments, z is 0. In some embodiments, z is 1.

In some embodiments, each R⁷ is independently C₁-C₆ alkyl, halo, C₁-C₆ haloalkyl, or —OH, or two R⁷ groups are taken together to form an oxo group. In some embodiments, each R⁷ is independently C₁-C₃ alkyl, halo, C₁-C₃ haloalkyl, or —OH, or two R⁷ groups are taken together to form an oxo group. In some embodiments, each R⁷ is independently —CH₃, —CF₃, F, or —OH, or two R⁷ groups are taken together to form an oxo group.

In some embodiments, R⁷ is C₁-C₆ alkyl. In some embodiments, R⁷ is C₁-C₃ alkyl. In some embodiments, R⁷ is methyl, ethyl, n-propyl, or isopropyl. In some embodiments, R⁷ is methyl. In some embodiments, R⁷ is ethyl. In some embodiments, R⁷ is n-propyl. In some embodiments, R⁷ is isopropyl.

In some embodiments, R⁷ is halo. In some embodiments, R⁷ is Cl, F, or Br. In some embodiments, R⁷ is C₁. In some embodiments, R⁷ is F. In some embodiments, R⁷ is Br.

In some embodiments, R⁷ is C₁-C₆ haloalkyl. In some embodiments, R⁷ is C₁-C₆ haloalkyl containing 1-13 halogen atoms. In some embodiments, R⁷ is C₁-C₃ haloalkyl. In some embodiments, R⁷ is C₁-C₃ haloalkyl containing 1-7 halogen atoms. In some embodiments, R⁷ is —CF₃, —CHF₂, —CH₂F, —CCl₃, —CHCl₂, —CH₂C₁, —CF₂C₁, —CFCl₂, —CH₂CF₃, —CH₂CHF₂, or —CH₂CCl₃. In some embodiments, R⁷ is —CF₃. In some embodiments, R⁷ is —CHF₂.

In some embodiments, R⁷ is —OH.

In some embodiments, two R⁷ groups are taken together to form an oxo group.

In some embodiments, each R¹¹ and R¹² is independently H, halo, C₃-C₆ cycloalkyl, —OH, —NH(C₁-C₆ alkyl), C₁-C₆ haloalkyl, or C₁-C₆ alkyl. In some embodiments, each R¹¹ and R¹² is independently H or C₁-C₃ alkyl. In some embodiments, each R¹¹ and R¹² is independently H or —CH₃.

In some embodiments, R¹¹ is H. In some embodiments, R¹¹ is halo. In some embodiments, R¹¹ is Cl, F, or Br. In some embodiments, R¹¹ is Cl. In some embodiments, R¹¹ is F. In some embodiments, R¹¹ is Br.

In some embodiments, R¹¹ is C₃-C₆ cycloalkyl. In some embodiments, R¹¹ is C₃-C₅ cycloalkyl. In some embodiments, R¹¹ is cyclopropyl, cyclobutyl, cyclopentyl, or cyclohexyl. In some embodiments, R¹¹ is cyclopropyl. In some embodiments, R¹¹ is cyclobutyl.

In some embodiments, R¹¹ is —OH.

In some embodiments, R¹¹ is —NH(C₁-C₆ alkyl). In some embodiments, R¹¹ is —NH(C₁-C₃ alkyl). In some embodiments, R¹¹ is —NH(CH₃), —NH(CH₂CH₃), —NH(CH₂CH₂CH₃), or —NH(CH(CH₃)₂). In some embodiments, R¹¹ is —NH(CH₃).

In some embodiments, R¹¹ is C₁-C₆ haloalkyl. In some embodiments, R¹¹ is C₁-C₆ haloalkyl containing 1-13 halogen atoms. In some embodiments, R¹¹ is C₁-C₃ haloalkyl. In some embodiments, R¹¹ is C₁-C₃ haloalkyl containing 1-7 halogen atoms. In some embodiments, R¹¹ is —CF₃, —CHF₂, —CH₂F, —CCl₃, —CHCl₂, —CH₂C₁, —CF₂C₁, —CFCl₂, —CH₂CF₃, —CH₂CHF₂, or —CH₂CCl₃. In some embodiments, R¹¹ is —CF₃. In some embodiments, R¹¹ is —CHF₂.

In some embodiments, R¹¹ is C₁-C₆ alkyl. In some embodiments, R¹¹ is C₁-C₃ alkyl. In some embodiments, R¹¹ is methyl, ethyl, n-propyl, or isopropyl. In some embodiments, R¹¹ is methyl. In some embodiments, R¹¹ is ethyl. In some embodiments, R¹¹ is n-propyl. In some embodiments, R¹¹ is isopropyl.

In some embodiments, R¹² is H.

In some embodiments, R¹² is halo. In some embodiments, R¹² is Cl, F, or Br. In some embodiments, R¹² is C₁. In some embodiments, R¹² is F. In some embodiments, R¹² is Br.

In some embodiments, R¹² is C₃-C₆ cycloalkyl. In some embodiments, R¹² is C₃-C₅ cycloalkyl. In some embodiments, R¹² is cyclopropyl, cyclobutyl, cyclopentyl, or cyclohexyl. In some embodiments, R¹² is cyclopropyl. In some embodiments, R¹² is cyclobutyl.

In some embodiments, R¹² is —OH.

In some embodiments, R¹² is —NH(C₁-C₆ alkyl). In some embodiments, R¹² is —NH(C₁-C₃ alkyl). In some embodiments, R¹² is —NH(CH₃), —NH(CH₂CH₃), —NH(CH₂CH₂CH₃), or —NH(CH(CH₃)₂). In some embodiments, R¹² is —NH(CH₃).

In some embodiments, R¹² is C₁-C₆ haloalkyl. In some embodiments, R¹² is C₁-C₆ haloalkyl containing 1-13 halogen atoms. In some embodiments, R¹² is C₁-C₃ haloalkyl. In some embodiments, R¹² is C₁-C₃ haloalkyl containing 1-7 halogen atoms. In some embodiments, R¹² is —CF₃, —CHF₂, —CH₂F, —CCl₃, —CHCl₂, —CH₂C₁, —CF₂C₁, —CFCl₂, —CH₂CF₃, —CH₂CHF₂, or —CH₂CCl₃. In some embodiments, R¹² is —CF₃. In some embodiments, R¹² is —CHF₂.

In some embodiments, R¹² is C₁-C₆ alkyl. In some embodiments, R¹² is C₁-C₃ alkyl. In some embodiments, R¹² is methyl, ethyl, n-propyl, or isopropyl. In some embodiments, R¹² is methyl. In some embodiments, R¹² is ethyl. In some embodiments, R¹² is n-propyl. In some embodiments, R¹² is isopropyl.

In some embodiments, R¹¹ and R¹² are each H. In some embodiments, R¹¹ and R¹² are each —CH₃. In some embodiments, one of R¹¹ and R¹² is H, and the other of R¹¹ and R¹² is —CH₃.

In some embodiments, R¹¹ and R³ are taken together to form a C₃-C₆ cycloalkylene group. In some embodiments, R¹¹ and R³ are taken together to form a C₃-C₅ cycloalkylene group. In some embodiments, R¹¹ and R³ are taken together to form a C₄-C₆ cycloalkylene group. In some embodiments, R¹¹ and R³ are taken together to form cyclopropylene, cyclobutylene, cyclopentylene, or cyclohexylene. In some embodiments, R¹¹ and R³ are taken together to form cyclopropylene.

In some embodiments, L³ is:

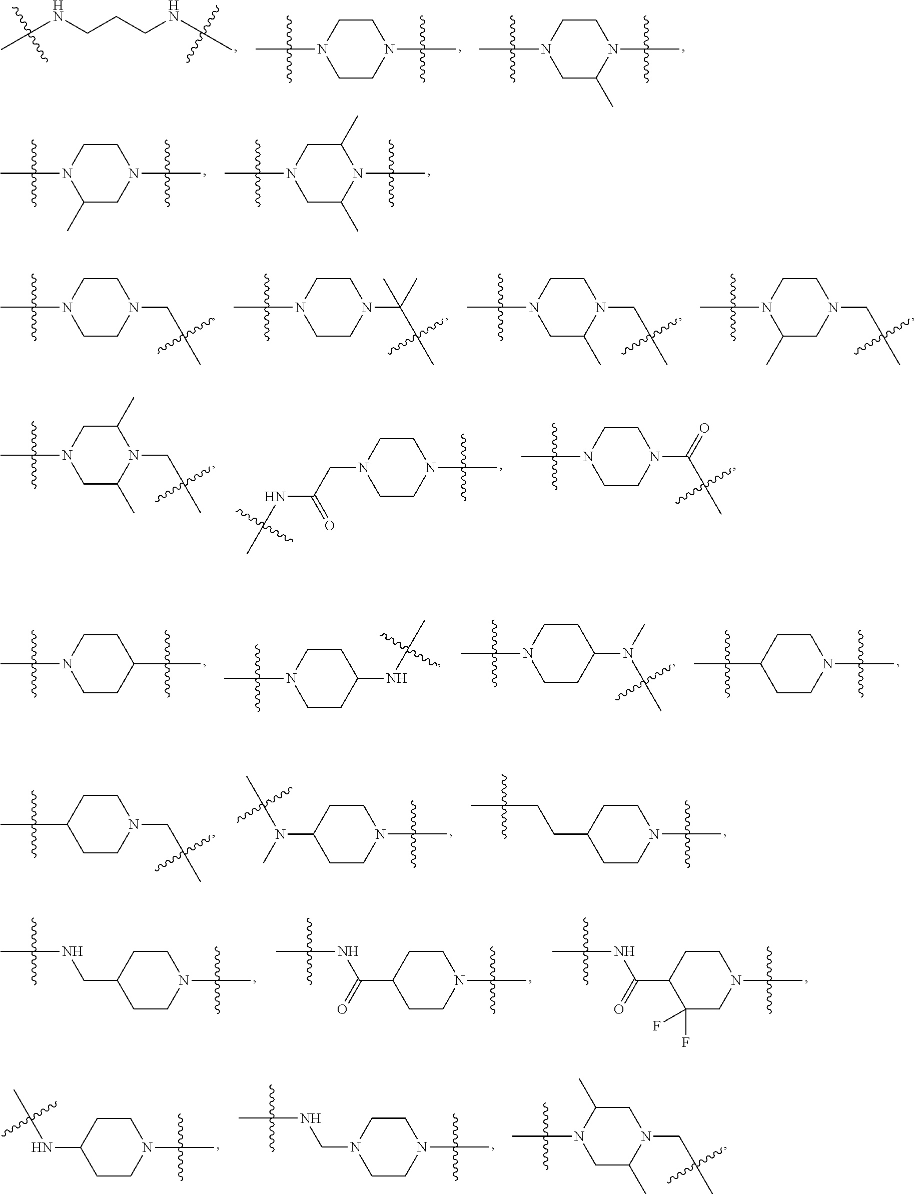

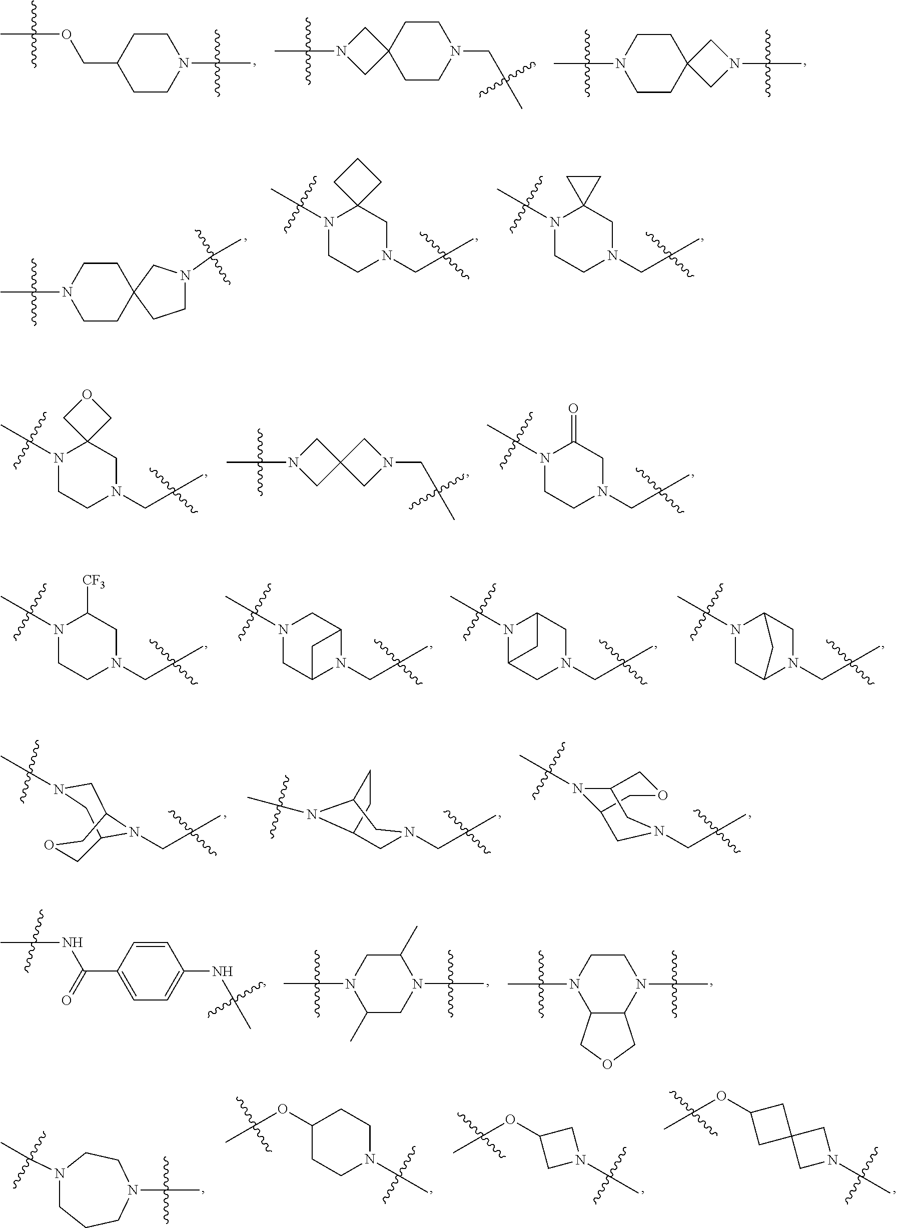

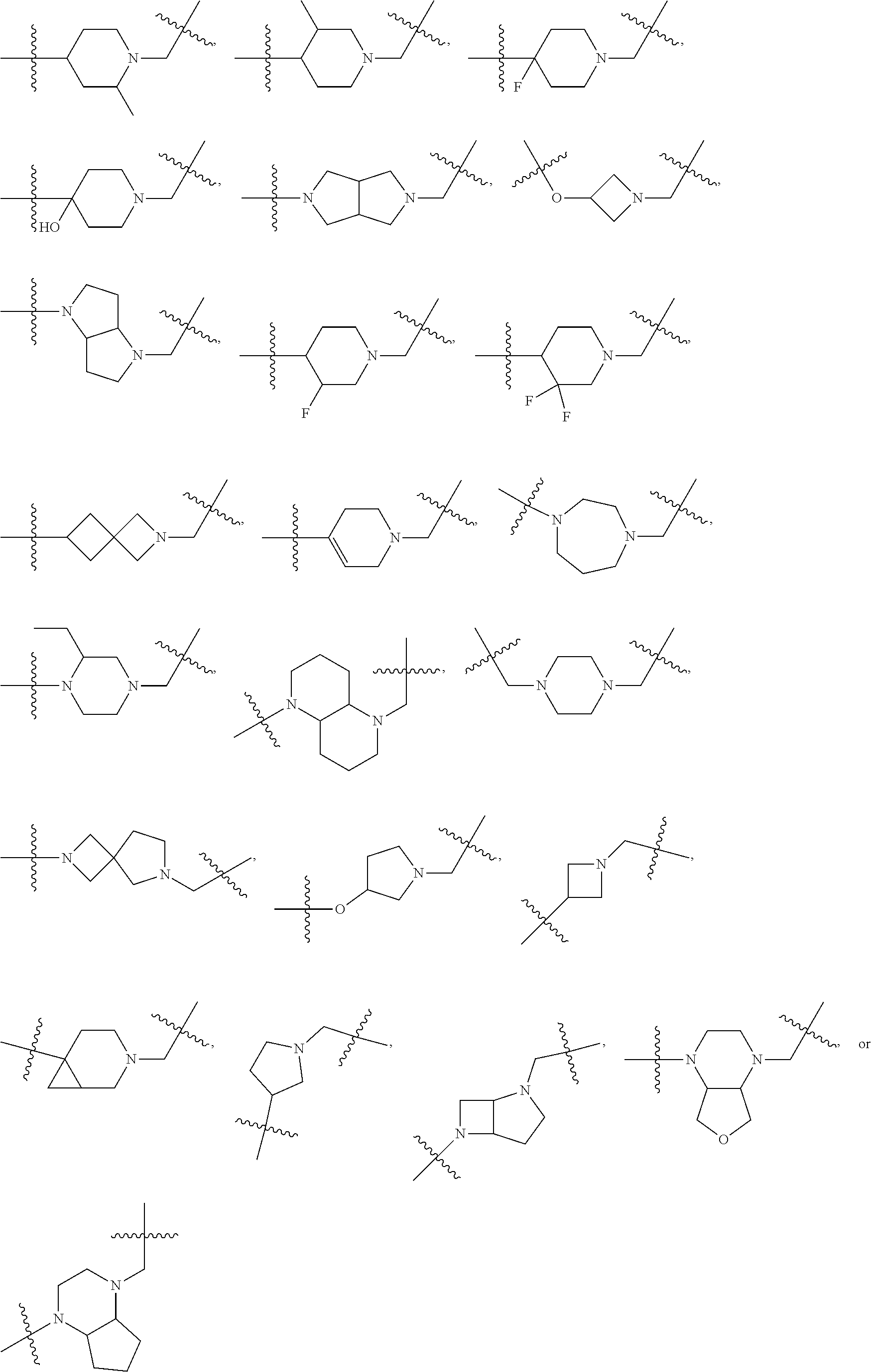

In some embodiments, L⁴ is

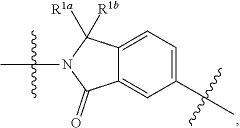

phenylene, —N(H)(phenylene), 5- to 6-membered heteroarylene, —N(H)(5- to 6-membered heteroarylene)-, 8- to 10-membered fused bicyclic heteroarylene, or 5- to 6-membered heterocyclylene, wherein the phenylene, heteroarylene, or heterocyclylene is optionally substituted by 1-4 R¹⁰ groups, and wherein the heteroarylene and heterocyclylene contain 1-3 heteroatoms selected from N, S, and O.

In some embodiments, L⁴ is

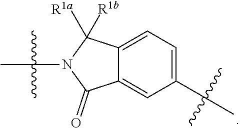

In some embodiments, R^{1a} and R^{1b} are each H or are taken together to form an oxo group. In some embodiments, R^{1a} and R^{1b} are each H. In some embodiments, R^{1a} and R^{1b} are taken together to form an oxo group. In some embodiments, L⁴ is

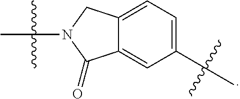 [SMILES: *c1ccc2c(c1)C(=O)N(*)C2]

In some embodiments, L⁴ is

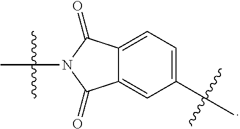 [SMILES: *c1ccc2c(c1)C(=O)N(*)C2=O]

In some embodiments, L⁴ is phenylene, —NH(phenylene), 5- to 6-membered heteroarylene, —N(H)(5- to 6-membered heteroarylene)-, 8- to 10-membered fused bicyclic heteroarylene, or 5- to 6-membered heterocyclylene, each of which is optionally substituted by 1-4 R¹⁰ groups, and wherein the heteroarylene or heterocyclylene contains 1-3 heteroatoms selected from N, S, and O.

In some embodiments, L⁴ is phenylene optionally substituted by 1-4 R¹⁰ groups. In some embodiments, L⁴ is phenylene optionally substituted by 1-3 R¹⁰ groups. In some embodiments, L⁴ is phenylene optionally substituted by 1-2 R¹⁰ groups. In some embodiments, L⁴ is phenylene optionally substituted by 1 R¹⁰ group. In some embodiments, L⁴ is unsubstituted phenylene.

In some embodiments, L⁴ is —N(H)phenylene optionally substituted by 1-4 R¹⁰ groups. In some embodiments, L⁴ is —N(H)phenylene optionally substituted by 1-3 R¹⁰ groups. In some embodiments, L⁴ is —N(H)phenylene optionally substituted by 1-2 R¹⁰ groups. In some embodiments, L⁴ is —N(H)phenylene optionally substituted by 1 R¹⁰ group. In some embodiments, L⁴ is unsubstituted —N(H)phenylene.

In some embodiments, L⁴ is 5- to 6-membered heteroarylene optionally substituted by 1-4 R¹⁰ groups, and wherein the heteroarylene contains 1-3 heteroatoms selected from N, S, and O. In some embodiments, L⁴ is 5- to 6-membered heteroarylene optionally substituted by 1-4 R¹⁰ groups, and wherein the heteroarylene contains 1-3 nitrogen atoms. In some embodiments, L⁴ is 5-membered heteroarylene optionally substituted by 1-4 R¹⁰ groups, and wherein the heteroarylene contains 1-3 nitrogen atoms. In some embodiments, L⁴ is 6-membered heteroarylene optionally substituted by 1-4 R¹⁰ groups, and wherein the heteroarylene contains 1-3 nitrogen atoms. In some embodiments, L⁴ is 5- to 6-membered heteroarylene optionally substituted by 1-4 R¹⁰ groups, and wherein the heteroarylene contains 2 heteroatoms selected from N and S. In some embodiments, L⁴ is 5-membered heteroarylene optionally substituted by 1-4 R¹⁰ groups, and wherein the heteroarylene contains 1 nitrogen atom and 1 sulfur atom. In some embodiments, the 5- to 6-membered heteroarylene is pyridinylene, pyrimidinylene, pyrazinylene, pyridazinylene, triazolylene, imidazolylene, thiazolylene, pyrazolylene, or pyrrolylene, each of which is optionally substituted by 1-4 R¹⁰ groups.

In some embodiments, L⁴ is 8- to 10-membered fused bicyclic heteroarylene optionally substituted by 1-4 R¹¹ groups, and wherein the fused heteroarylene contains 1-3 heteroatoms selected from N, S, and O. In some embodiments, L⁴ is 8- to 10-membered fused bicyclic heteroarylene optionally substituted by 1-4 R¹⁰ groups, and wherein the fused heteroarylene contains 1-3 nitrogen atoms. In some embodiments, L⁴ is 8- to 10-membered fused bicyclic heteroarylene optionally substituted by 1-4 R¹¹ groups, and wherein the fused heteroarylene contains 1-3 nitrogen atoms. In some embodiments, L⁴ is 8-membered fused heteroarylene optionally substituted by 1-4 R¹⁰ groups, and wherein the heteroarylene contains 1-3 nitrogen atoms. In some embodiments, L⁴ is 9-membered fused heteroarylene optionally substituted by 1-4 R¹⁰ groups, and wherein the heteroarylene contains 1-3 nitrogen atoms. In some embodiments, L⁴ is 10-membered fused heteroarylene optionally substituted by 1-4 R¹⁰ groups, and wherein the heteroarylene contains 1-3 nitrogen atoms.

In some embodiments, L⁴ is —N(H)(5- to 6-membered heteroarylene) optionally substituted by 1-4 R¹⁰ groups, and wherein the heteroarylene contains 1-3 heteroatoms selected from N, S, and O. In some embodiments, L⁴ is —N(H)(5- to 6-membered heteroarylene) optionally substituted by 1-4 R¹¹ groups, and wherein the heteroarylene contains 1-3 nitrogen atoms. In some embodiments, L⁴ is —N(H)(5-membered heteroarylene) optionally substituted by 1-4 R¹¹ groups, and wherein the heteroarylene contains 1-3 nitrogen atoms. In some embodiments, L⁴ is —N(H)(6-membered heteroarylene) optionally substituted by 1-4 R¹¹ groups, and wherein the heteroarylene contains 1-3 nitrogen atoms. In some embodiments, the 5- to 6-membered heteroarylene is pyridinylene, pyrimidinylene, pyrazinylene, pyridazinylene, triazolylene, imidazolylene, thiazolylene, pyrazolylene, or pyrrolylene, each of which is optionally substituted by 1-4 R¹¹ groups.

In some embodiments, L⁴ is 5- to 6-membered heterocyclylene optionally substituted by 1-4 R¹⁰ groups, and wherein the heterocyclylene contains 1-3 heteroatoms selected from N and O. In some embodiments, L⁴ is 5-membered heterocyclylene optionally substituted by 1-4 R¹⁰ groups, and wherein the heterocyclylene contains 1-3 heteroatoms selected from N and O. In some embodiments, L⁴ is 6-membered heterocyclylene optionally substituted by 1-4 R¹⁰ groups, and wherein the heterocyclylene contains 1-3 heteroatoms selected from N and O. In some embodiments, L⁴ is 5- to 6-membered heterocyclylene optionally substituted by 1-4 R¹⁰ groups, and wherein the heterocyclylene contains 1-2 heteroatoms selected from N and O. In some embodiments, L⁴ is 5- to 6-membered heterocyclylene optionally substituted by 1-4 R¹⁰ groups, and wherein the heterocyclylene contains 1-2 nitrogen atoms. In some embodiments, L⁴ is 5- to 6-membered heterocyclylene optionally substituted by 1-4 R¹⁰ groups, and wherein the heterocyclylene contains one nitrogen atom.

In some embodiments, each R¹⁰ is independently C₁-C₆ alkoxy, C₁-C₆ alkyl, halo, or —OH, or two R¹⁰ groups are taken together to form an oxo group. In some embodiments, each It m is independently C₁-C₃ alkoxy, C₁-C₃ alkyl, halo, or —OH, or two R¹⁰ groups are taken together to form an oxo group. In some embodiments, each R¹⁰ is independently —OCH₃, —CH₃, Cl, F, or —OH, or two R¹⁰ groups are taken together to form an oxo group.

In some embodiments, R¹⁰ is C₁-C₆ alkoxy. In some embodiments, R¹⁰ is C₁-C₃ alkoxy. In some embodiments, R¹⁰ is —OCH₃, —OCH₂CH₃, —OCH₂CH₂CH₃, or —OCH(CH₃)₂. In some embodiments, R¹⁰ is —OCH₃. In some embodiments, R¹⁰ is —OCH₂CH₃.

In some embodiments, R¹⁰ is C₁-C₆ alkyl. In some embodiments, R¹⁰ is C₁-C₃ alkyl. In some embodiments, R¹⁰ is methyl, ethyl, n-propyl, or isopropyl. In some embodiments, R¹⁰ is methyl. In some embodiments, R¹⁰ is ethyl. In some embodiments, R¹⁰ is n-propyl. In some embodiments, R¹⁰ is isopropyl.

In some embodiments, R¹⁰ is halo. In some embodiments, R¹⁰ is Cl, F, or Br. In some embodiments, R¹⁰ is C₁. In some embodiments, R¹⁰ is F. In some embodiments, R¹⁰ is Br.

In some embodiments, R¹⁰ is —OH.

In some embodiments, two R¹⁰ groups are taken together to form an oxo group. In some embodiments, L⁴ is:

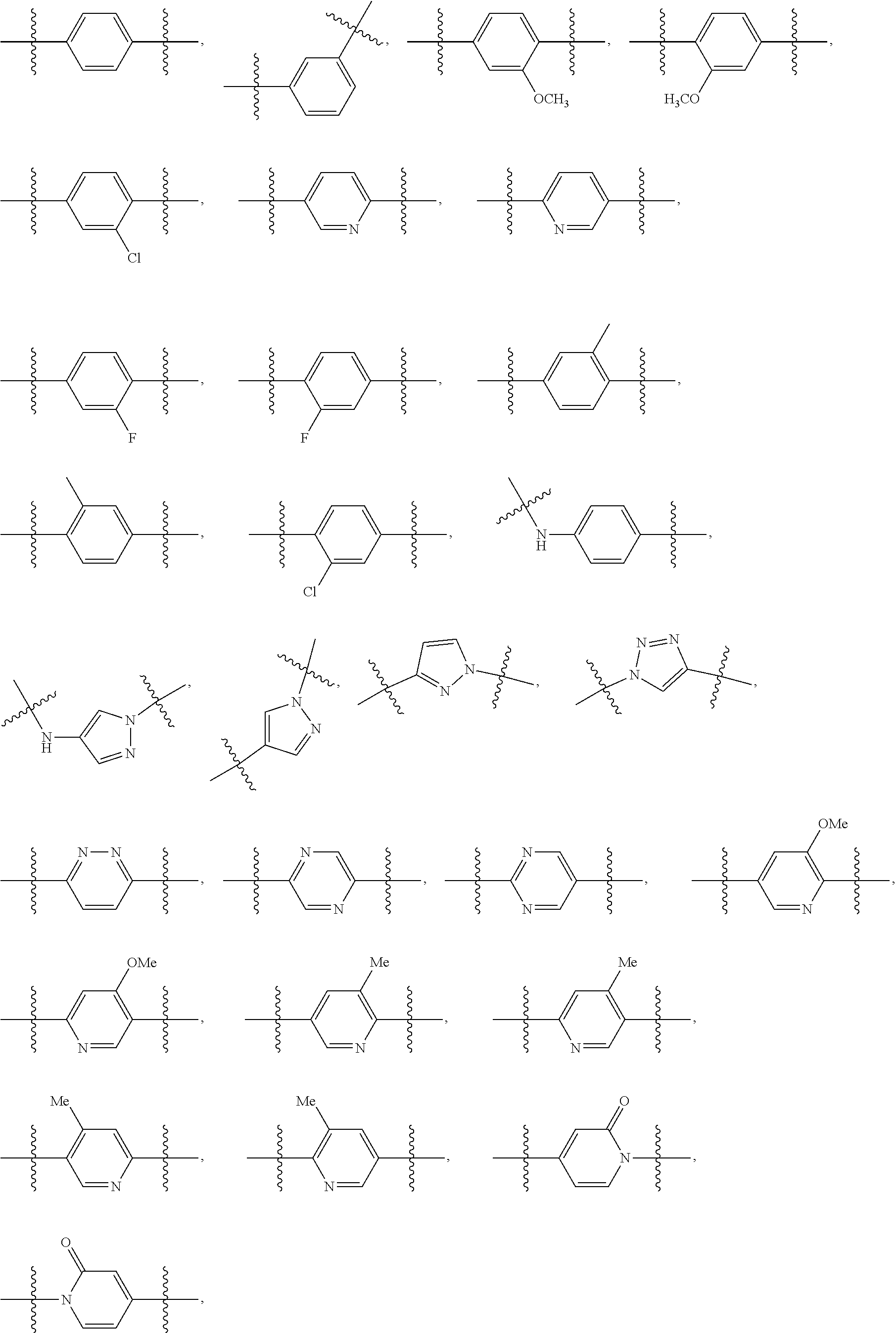

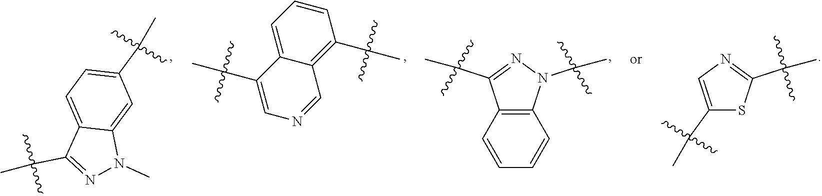 [SMILES: *c1ccc2c(*)nn(C)c2c1 ; *c1cncc2c(*)cccc12 ; *c1nn(*)c2ccccc12 ; *c1cnc(*)s1]

In some embodiments, R² and R³ are independently H, C₁-C₆ alkyl, or halo, or R² and R³ are taken together to form an oxo group. In some embodiments, R² and R³ are independently H, C₁-C₃ alkyl, or halo. In some embodiments, R² and R³ are independently H, —CH₃, or F. In some embodiments, R² and R³ are taken together to form an oxo group.

In some embodiments, R² is H.

In some embodiments, R² is C₁-C₆ alkyl. In some embodiments, R² is C₁-C₃ alkyl. In some embodiments, R² is methyl, ethyl, n-propyl, or isopropyl. In some embodiments, R² is methyl. In some embodiments, R² is ethyl. In some embodiments, R² is n-propyl. In some embodiments, R² is isopropyl.

In some embodiments, R² is halo. In some embodiments, R² is Cl, F, or Br. In some embodiments, R² is C₁. In some embodiments, R² is F. In some embodiments, R² is Br.

In some embodiments, R³ is H.

In some embodiments, R³ is C₁-C₆ alkyl. In some embodiments, R³ is C₁-C₃ alkyl. In some embodiments, R³ is methyl, ethyl, n-propyl, or isopropyl. In some embodiments, R³ is methyl. In some embodiments, R³ is ethyl. In some embodiments, R³ is n-propyl. In some embodiments, R³ is isopropyl.

In some embodiments, R³ is halo. In some embodiments, R³ is Cl, F, or Br. In some embodiments, R³ is C₁. In some embodiments, R³ is F. In some embodiments, R³ is Br.

In some embodiments, R² and R³ are taken together to form an oxo group.

In some embodiments, R³ and R¹¹ are taken together to form a C₃-C₆ cycloalkylene group. In some embodiments, R³ and R¹¹ are taken together to form a C₃-C₅ cycloalkylene group. In some embodiments, R³ and R¹¹ are taken together to form a C₄-C₆ cycloalkylene group. In some embodiments, R³ and R¹¹ are taken together to form cyclopropylene, cyclobutylene, cyclopentylene, or cyclohexylene. In some embodiments, R³ and R¹¹ are taken together to form cyclopropylene.

In some embodiments, x is 0 or 1. In some embodiments, x is 0. In some embodiments, x is 1.

In some embodiments, Y is NH, O, or a bond. In some embodiments, Y is NH. In some embodiments, Y is O. In some embodiments, Y is a bond.

In some embodiments, R⁴ is C₃-C₆ cycloalkyl, C₁-C₆ alkylene-(C₃-C₆ cycloalkyl), 4- to 6-membered heterocyclyl, C₁-C₆ alkylene-(4- to 6-membered heterocyclyl), 5- to 6-membered heteroaryl, C₁-C₆ alkylene-(5- to 6-membered heteroaryl), C₁-C₆ alkyl, C₁-C₆ haloalkyl, C₁-C₆ alkyl-OH, or C₁-C₆ alkyl-CN, wherein the heterocyclyl and heteroaryl contain 1-3 heteroatoms selected from N and O, and wherein the cycloalkyl, heterocyclyl, or heteroaryl is optionally substituted by 1-5 R⁸ groups. In some embodiments, R⁴ is C₃-C₆ cycloalkyl, C₁-C₃ alkylene-(C₃-C₆ cycloalkyl), 4- to 6-membered heterocyclyl, C₁-C₃ alkylene-(4- to 6-membered heterocyclyl), 5- to 6-membered heteroaryl, C₁-C₃ alkylene-(5- to 6-membered heteroaryl), C₁-C₆ alkyl, C₁-C₆ haloalkyl, C₁-C₆ alkyl-OH, or C₁-C₆ alkyl-CN, wherein the heterocyclyl and heteroaryl contain 1 or 2 heteroatoms selected from N and O, and wherein the cycloalkyl, heterocyclyl, or heteroaryl is optionally substituted by 1-2 R⁸ groups.

In some embodiments, R⁴ is C₃-C₆ cycloalkyl optionally substituted by 1-5 R⁸ groups. In some embodiments, R⁴ is C₃-C₅ cycloalkyl optionally substituted by 1-5 R⁷ groups. In some embodiments, R⁴ is cyclopropyl, cyclobutyl, cyclopentyl, or cyclohexyl, each of which is optionally substituted by 1-5 R⁷ groups. In some embodiments, R⁴ is cyclopropyl optionally substituted by 1-5 R⁷ groups. In some embodiments, R⁴ is cyclobutyl optionally substituted by 1-5 R⁷ groups. In some embodiments, R⁴ is cyclopentyl optionally substituted by 1-5 R⁷ groups. In some variations, R⁴ is C₃-C₆ cycloalkyl substituted by 1-5 R⁷ groups. In some variations, R⁴ is C₃-C₆ cycloalkyl substituted by 1-2 R⁸ groups. In some embodiments, R⁴ is unsubstituted C₃-C₆ cycloalkyl.

In some embodiments, R⁴ is C₁-C₆ alkylene-(C₃-C₆ cycloalkyl), wherein the cycloalkyl is optionally substituted by 1-5 R⁷ groups. In some embodiments, R⁴ is C₁-C₃ alkylene-(C₃-C₆ cycloalkyl), wherein the cycloalkyl is optionally substituted by 1-5 R⁸ groups.

In some embodiments, R⁴ is C₁-C₃ alkylene-(cyclopropyl), wherein the cyclopropyl is optionally substituted by 1-5 R⁷ groups. In some embodiments, R⁴ is C₁-C₃ alkylene-(cyclobutyl), wherein the cyclobutyl is optionally substituted by 1-5 R⁷ groups. In some embodiments, R⁴ is C₁-C₃ alkylene-(cyclopentyl), wherein the cyclopentyl is optionally substituted by 1-5 R⁷ groups. In some embodiments, R⁴ is C₁-C₃ alkylene-(cyclohexyl), wherein the cyclohexyl is optionally substituted by 1-5 R⁷ groups. In some embodiments, R⁴ is —CH₂—(C₃-C₆ cycloalkyl), wherein the cycloalkyl is optionally substituted by 1-5 R⁸ groups. In some embodiments, R⁴ is —CH₂CH₂—(C₃-C₆ cycloalkyl), wherein the cycloalkyl is optionally substituted by 1-5 R⁷ groups.

In some embodiments, R⁴ is 4- to 6-membered heterocyclyl optionally substituted by 1-5 R⁸ groups, wherein the heterocyclyl contains 1-3 heteroatoms selected from N and O. In some embodiments, R⁴ is 4- to 6-membered heterocyclyl optionally substituted by 1-5 R⁸ groups, wherein the heterocyclyl contains 1-2 heteroatoms selected from N and O. In some embodiments, R⁴ is 4- to 6-membered heterocyclyl optionally substituted by 1-5 R⁸ groups, wherein the heterocyclyl contains one nitrogen atom. In some embodiments, R⁴ is 4- to 6-membered heterocyclyl optionally substituted by 1-5 R⁸ groups, wherein the heterocyclyl contains one oxygen atom. In some variations, R⁴ is 4- to 6-membered heterocyclyl substituted by 1-3 R⁸ groups, wherein the heterocyclyl contains 1-3 heteroatoms selected from N and O. In some variations, R⁴ is 4- to 6-membered heterocyclyl substituted by one R⁸ group, wherein the heterocyclyl contains 1-3 heteroatoms selected from N and O. In some variations, R⁴ is unsubstituted 4- to 6-membered heterocyclyl, wherein the heterocyclyl contains 1-3 heteroatoms selected from N and O. In some embodiments, the 4- to 6-membered heterocyclyl is oxetanyl, tetrahydrofuranyl, tetrahydropyranyl, piperidinyl, pyrrolidinyl, azetidinyl, or piperazinyl, each of which is optionally substituted by 1-5 R⁷ groups.

In some embodiments, R⁴ is C₁-C₆ alkylene-(4- to 6-membered heterocyclyl), wherein the heterocyclyl is optionally substituted by 1-5 R⁷ groups, and wherein the heterocyclyl contains 1-3 heteroatoms selected from N and O. In some embodiments, R⁴ is C₁-C₃ alkylene-(4- to 6-membered heterocyclyl), wherein the heterocyclyl is optionally substituted by 1-5 R⁷ groups, and wherein the heterocyclyl contains 1-3 heteroatoms selected from N and O. In some embodiments, R⁴ is —CH₂(4- to 6-membered heterocyclyl), wherein the heterocyclyl is optionally substituted by 1-5 R⁷ groups, and wherein the heterocyclyl contains 1-3 heteroatoms selected from N and O. In some embodiments, R⁴ is —CH₂CH₂(4- to 6-membered heterocyclyl), wherein the heterocyclyl is optionally substituted by 1-5 R⁷ groups, and wherein the heterocyclyl contains 1-3 heteroatoms selected from N and O. In some embodiments, the 4- to 6-membered heterocyclyl contains 1-2 heteroatoms selected from N and O. In some embodiments, the 4- to 6-membered heterocyclyl contains one nitrogen atom. In some embodiments, the 4- to 6-membered heterocyclyl contains one oxygen atom. In some variations, the 4- to 6-membered heterocyclyl is substituted by 1-3 R⁸ groups. In some variations, the 4- to 6-membered heterocyclyl is substituted by one R⁸ group. In some variations, the 4- to 6-membered heterocyclyl is unsubstituted. In some embodiments, the 4- to 6-membered heterocyclyl is oxetanyl, tetrahydrofuranyl, tetrahydropyranyl, piperidinyl, pyrrolidinyl, azetidinyl, or piperazinyl, each of which is optionally substituted by 1-5 R⁷ groups.

In some embodiments, R⁴ is 5- to 6-membered heteroaryl, wherein the heteroaryl is optionally substituted by 1-5 R⁸ groups, and wherein the heteroaryl contains 1-3 heteroatoms selected from N and O. In some embodiments, R⁴ is 5-membered heteroaryl, wherein the heteroaryl is optionally substituted by 1-5 R⁸ groups, and wherein the heteroaryl contains 1-3 heteroatoms selected from N and O. In some embodiments, R⁴ is 6-membered heteroaryl, wherein the heteroaryl is optionally substituted by 1-5 R⁸ groups, and wherein the heteroaryl contains 1-3 heteroatoms selected from N and O. In some embodiments, R⁴ is 5- to 6-membered heteroaryl, wherein the heteroaryl is optionally substituted by 1-5 R⁸ groups, and wherein the heteroaryl contains 1-2 heteroatoms selected from N and O. In some embodiments, R⁴ is 5- to 6-membered heteroaryl, wherein the heteroaryl is optionally substituted by 1-5 R⁸ groups, and wherein the heteroaryl contains 1-2 nitrogen atoms. In some variations, the 5- to 6-membered heteroaryl is substituted by 1-3 R⁸ groups. In some variations, the 5- to 6-membered heteroaryl is substituted by one R⁸ group. In some variations, the 5- to 6-membered heteroaryl is unsubstituted. In some embodiments, the 5- to 6-membered heteroaryl is piperidinyl, pyrazinyl, pyrimidinyl, pyridazinyl, pyrrolyl, pyrazolyl, or imidazolyl, each of which is optionally substituted by 1-5 R⁷ groups.

In some embodiments, R⁴ is C₁-C₆ alkylene-(5- to 6-membered heteroaryl), wherein the heteroaryl is optionally substituted by 1-5 R⁷ groups, and wherein the heteroaryl contains 1-3 heteroatoms selected from N and O. In some embodiments, R⁴ is C₁-C₆ alkylene-(5-membered heteroaryl), wherein the heteroaryl is optionally substituted by 1-5 R⁷ groups, and wherein the heteroaryl contains 1-3 heteroatoms selected from N and O. In some embodiments, R⁴ is C₁-C₆ alkylene-(6-membered heteroaryl), wherein the heteroaryl is optionally substituted by 1-5 R⁸ groups, and wherein the heteroaryl contains 1-3 heteroatoms selected from N and O. In some embodiments, R⁴ is C₁-C₃ alkylene-(5- to 6-membered heteroaryl), wherein the heteroaryl is optionally substituted by 1-5 R⁷ groups, and wherein the heteroaryl contains 1-3 heteroatoms selected from N and O. In some embodiments, R⁴ is —CH₂-(5- to 6-membered heteroaryl), wherein the heteroaryl is optionally substituted by 1-5 R⁷ groups, and wherein the heteroaryl contains 1-3 heteroatoms selected from N and O. In some embodiments, R⁴ is —CH₂CH₂-(5- to 6-membered heteroaryl), wherein the heteroaryl is optionally substituted by 1-5 R⁷ groups, and wherein the heteroaryl contains 1-3 heteroatoms selected from N and O. In some embodiments, R⁴ is C₁-C₃ alkylene-(5- to 6-membered heteroaryl), wherein the heteroaryl is optionally substituted by 1-5 R⁷ groups, and wherein the heteroaryl contains 1-2 heteroatoms selected from N and O. In some embodiments, R⁴ is C₁-C₃ alkylene-(5- to 6-membered heteroaryl), wherein the heteroaryl is optionally substituted by 1-5 R⁷ groups, and wherein the heteroaryl contains 1-2 nitrogen atoms. In some variations, the 5- to 6-membered heteroaryl is substituted by 1-3 R⁸ groups. In some variations, the 5- to 6-membered heteroaryl is substituted by one R⁸ group. In some variations, the 5- to 6-membered heteroaryl is unsubstituted. In some embodiments, the 5- to 6-membered heteroaryl is piperidinyl, pyrazinyl, pyrimidinyl, pyridazinyl, pyrrolyl, pyrazolyl, or imidazolyl, each of which is optionally substituted by 1-5 R⁸ groups.

In some embodiments, R⁴ is C₁-C₆ alkyl. In some embodiments, R⁴ is C₁-C₃ alkyl. In some embodiments, R⁴ is C₁-C₄ alkyl. In some embodiments, R⁴ is methyl, ethyl, n-propyl, isopropyl, tert-butyl, or —CH₂CH(CH₃)₂. In some embodiments, R⁴ is methyl. In some embodiments, R⁴ is ethyl. In some embodiments, R⁴ is n-propyl. In some embodiments, R⁴ is isopropyl. In some embodiments, R⁴ is tert-butyl. In some embodiments, R⁴ is —CH₂CH(CH₃)₂.

In some embodiments, R⁴ is C₁-C₆ haloalkyl. In some embodiments, R⁴ is C₁-C₆ haloalkyl containing 1-13 halogen atoms. In some embodiments, R⁴ is C₁-C₃ haloalkyl. In some embodiments, R⁴ is C₁-C₃ haloalkyl containing 1-7 halogen atoms. In some embodiments, R⁴ is —CF₃, —CHF₂, —CH₂F, —CCl₃, —CHCl₂, —CH₂C₁, —CF₂C₁, —CFCl₂, —CH₂CF₃, —CH₂CHF₂, or —CH₂CCl₃. In some embodiments, R⁴ is —CF₃. In some embodiments, R⁴ is —CHF₂. In some embodiments, R⁴ is C₂-C₃ haloalkyl containing 1-7 halogen atoms. In some embodiments, R⁴ is —CH₂CF₃, —CH₂CH₂F, —CH₂CF₂CH₃, —CH(CH₃)CF₃, or —CH₂CH₂CF₃.

In some embodiments, R⁴ is C₁-C₆ alkyl-OH. In some embodiments, R⁴ is C₁-C₃ alkyl-OH. In some embodiments, R⁴ is —CH₂OH, —CH₂CH₂OH, —CH₂CH₂CH₂OH, —CH(OH)CH₃, —CH(OH)CH₂OH, or —CH₂CH(OH)CH₃. In some embodiments, R⁴ is —CH₂OH. In some embodiments, R⁴ is —CH₂CH₂OH. In some embodiments, R⁴ is C₃-C₄ alkyl-OH. In some embodiments, R⁴ is —CH(CH₃)CH₂OH or —CH₂C(CH₃)₂OH.

In some embodiments, R⁴ is C₁-C₆ alkyl-CN. In some embodiments, R⁴ is C₁-C₃ alkyl-CN. In some embodiments, R⁴ is —CH₂CN, —CH₂CH₂CN, —CH₂CH₂CH₂CN, —CH(CH₃)CN, —C(CH₃)₂CN, or —CH₂CH(CN)CH₃. In some embodiments, R⁴ is —CH₂CN. In some embodiments, R⁴ is —CH₂CH₂CN. In some embodiments, R⁴ is —CH(CH₃)CN. In some embodiments, R⁴ is —C(CH₃)₂CN. In some embodiments, R⁴ is —CH(CH₂CH₃)CN or —CH₂CH(CH₃)CN.

In some embodiments, each R⁸ is independently —SO₂(C₁-C₆ alkyl), —C(O)(C₁-C₆ alkyl), C₁-C₆ alkyl, C₁-C₆ haloalkyl, halo, —CN, or —OH. In some embodiments, each R⁸ is independently —SO₂(C₁-C₃ alkyl), —C(O)(C₁-C₃ alkyl), C₁-C₃ alkyl, C₁-C₃ haloalkyl, halo, —CN, or —OH. In some embodiments, each R⁸ is independently —SO₂CH₃, —C(O)CH₃, methyl, ethyl, —CH₂CF₃, F, —CN, or —OH.

In some embodiments, R⁸ is —SO₂(C₁-C₆ alkyl). In some embodiments, R⁸ is —SO₂(C₁-C₃ alkyl). In some embodiments, R⁸ is —SO₂CH₃. In some embodiments, R⁸ is —SO₂CH₂CH₃. In some embodiments, R⁸ is —SO₂CH₂CH₂CH₃.

In some embodiments, R⁸ is —C(O)(C₁-C₆ alkyl). In some embodiments, R⁸ is —C(O)(C₁-C₃ alkyl). In some embodiments, R⁸ is —C(O)CH₃. In some embodiments, R⁸ is —C(O)CH₂CH₃. In some embodiments, R⁸ is —C(O)CH₂CH₂CH₃.

In some embodiments, R⁸ is C₁-C₆ alkyl. In some embodiments, R⁸ is C₁-C₃ alkyl. In some embodiments, R⁸ is methyl, ethyl, n-propyl, or isopropyl. In some embodiments, R⁸ is methyl. In some embodiments, R⁸ is ethyl. In some embodiments, R⁸ is n-propyl. In some embodiments, R⁸ is isopropyl.

In some embodiments, R⁸ is C₁-C₆ haloalkyl. In some embodiments, R⁸ is C₁-C₆ haloalkyl containing 1-13 halogen atoms. In some embodiments, R⁸ is C₁-C₃ haloalkyl. In some embodiments, R⁸ is C₁-C₃ haloalkyl containing 1-7 halogen atoms. In some embodiments, R⁸ is —CF₃, —CHF₂, —CH₂F, —CH₂CF₃, —CCl₃, —CHCl₂, —CH₂C₁, —CF₂C₁, —CFCl₂, —CH₂CF₃, —CH₂CHF₂, or —CH₂CCl₃. In some embodiments, R⁸ is —CF₃. In some embodiments, R⁸ is —CH₂CF₃.

In some embodiments, R⁸ is halo. In some embodiments, R⁸ is Cl, F, or Br. In some embodiments, R⁸ is C₁. In some embodiments, R⁸ is F. In some embodiments, R⁸ is Br.

In some embodiments, R⁸ is —CN.

In some embodiments, R⁸ is —OH.

In some embodiments, R⁴ is methyl, ethyl, n-propyl, isopropyl, tert-butyl, —CH₂CH(CH₃)₂, —CH₂CF₃, —CH₂CH₂F, —CH₂CF₂CH₃, —CH(CH₃)CF₃, —CH₂CH₂CF₃, —CH(CH₃)CH₂OH, —CH₂C(CH₃)₂OH, —CH₂CN, —CH(CH₃)CN, —C(CH₃)₂CN, —CH(CH₂CH₃)CN, —CH₂CH(CH₃)CN,

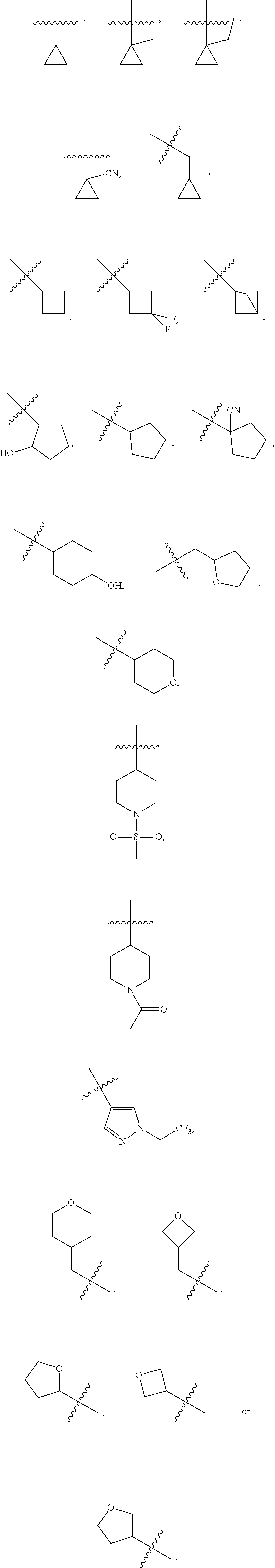

In some embodiments, W is O, —NR⁵—, or a bond. In some embodiments, W is O, —N(H)—, —N(CH₃)—, or a bond.

In some embodiments, W is O.

In some embodiments, W is a bond.

In some embodiments, W is —NR⁵—.

In some embodiments, R⁵ is H or C₁-C₆ alkyl. In some embodiments, R⁵ is H or C₁-C₃ alkyl.

In some embodiments, R⁵ is H.

In some embodiments, R⁵ is C₁-C₆ alkyl. In some embodiments, R⁵ is C₁-C₃ alkyl. In some embodiments, R⁵ is methyl, ethyl, n-propyl, or isopropyl. In some embodiments, R⁵ is methyl. In some embodiments, R⁵ is ethyl. In some embodiments, R⁵ is n-propyl. In some embodiments, R⁵ is isopropyl.

In some embodiments, W is —N(H)—. In some embodiments, W is —N(C₁-C₆ alkyl)-. In some embodiments, W is —N(CH₃)—.

In some embodiments, each R⁶ is independently C₁-C₆ alkyl, halo, or —OH, or two R⁶ groups are taken together to form a bridging C1-C₃ alkylene group. In some embodiments, each R⁶ is independently C₁-C₃ alkyl, halo, or —OH, or two R⁶ groups are taken together to form a bridging C1-C₂ alkylene group. In some embodiments, each R⁶ is independently —CH₃, C₁, or —OH, or two R⁶ groups are taken together to form a bridging C1-C₂ alkylene group.

In some embodiments, R⁶ is C₁-C₆ alkyl. In some embodiments, R⁶ is C₁-C₃ alkyl. In some embodiments, R⁶ is methyl, ethyl, n-propyl, or isopropyl. In some embodiments, R⁶ is methyl. In some embodiments, R⁶ is ethyl. In some embodiments, R⁶ is n-propyl. In some embodiments, R⁶ is isopropyl.

In some embodiments, R⁶ is halo. In some embodiments, R⁶ is Cl, F, or Br. In some embodiments, R⁶ is C₁. In some embodiments, R⁶ is F. In some embodiments, R⁶ is Br.

In some embodiments, R⁶ is —OH.

In some embodiments, two R⁶ groups are taken together to form a bridging C1-C₃ alkylene group. In some embodiments, two R⁶ groups are taken together to form a bridging C1-C₂ alkylene group. In some embodiments, two R⁶ groups are taken together to form a bridging methylene group. In some embodiments, two R⁶ groups are taken together to form a bridging ethylene group. In some embodiments, two R⁶ groups are taken together to form a bridging propylene group.

In some embodiments, y is 0, 1, 2, 3, 4, or 5. In some embodiments, y is 0 or 1. In some embodiments, y is 0. In some embodiments, y is 1. In some embodiments, y is 2. In some embodiments, y is 3. In some embodiments, y is 4. In some embodiments, y is 5.

In some embodiments,

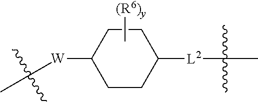

is:

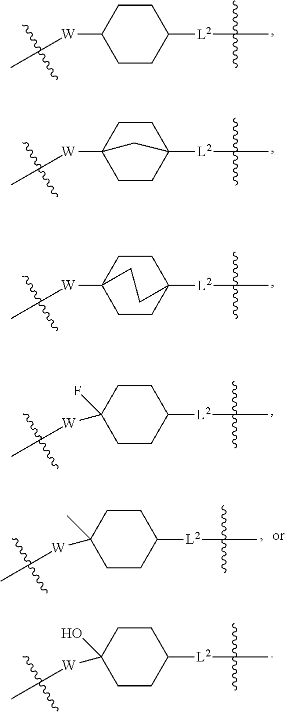

In some embodiments, X is N or CR¹³. In some embodiments, X is CR¹³; and R¹³ is H, halo, —OH, or C₁-C₃ alkyl.

In some embodiments, X is N.

In some embodiments, X is CR¹³.

In some embodiments, R¹³ is H, halo, —OH, or C₁-C₆ alkyl. In some embodiments, R¹³ is H, halo, —OH, or C₁-C₃ alkyl. In some embodiments, R¹³ is H, CH₃, —OH, or F.

In some embodiments, R¹³ is H.

In some embodiments, R¹³ is halo. In some embodiments, R¹³ is Cl, F, or Br. In some embodiments, R¹³ is C₁. In some embodiments, R¹³ is F. In some embodiments, R¹³ is Br.

In some embodiments, R¹³ is —OH.

In some embodiments, R¹³ is C₁-C₆ alkyl. In some embodiments, R¹³ is C₁-C₃ alkyl. In some embodiments, R¹³ is methyl, ethyl, n-propyl, or isopropyl. In some embodiments, R¹³ is methyl. In some embodiments, R¹³ is ethyl. In some embodiments, R¹³ is n-propyl. In some embodiments, R¹³ is isopropyl.

In some embodiments, the compound of Formula (I′) or (I) is a compound of Formula (IA), (IB), (IC), (ID), or (IE):

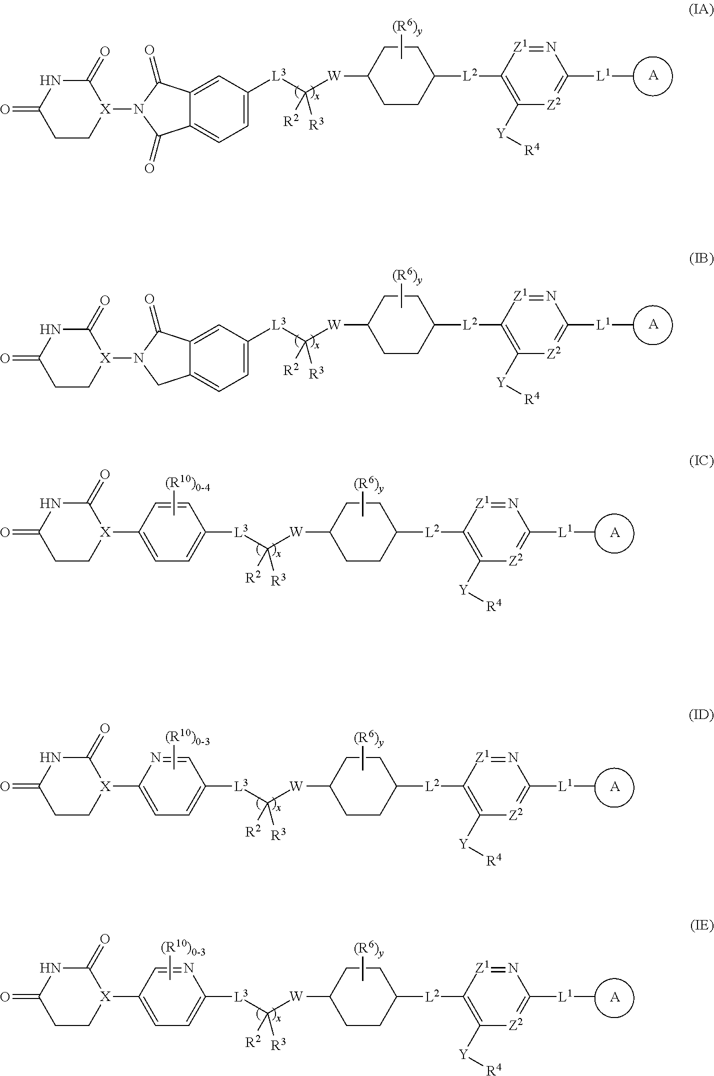

wherein Ring A, R², R³, R⁴, R⁶, R¹⁰, L¹, L², L³, Y, Z¹, Z², x, and y are as described for Formula (I′) or (I).

In some embodiments, the compound of Formula (I′) or (I) is a compound of Formula (IIa), (IIb), (IIIa), (IIIb), (IVa), or (IVb):

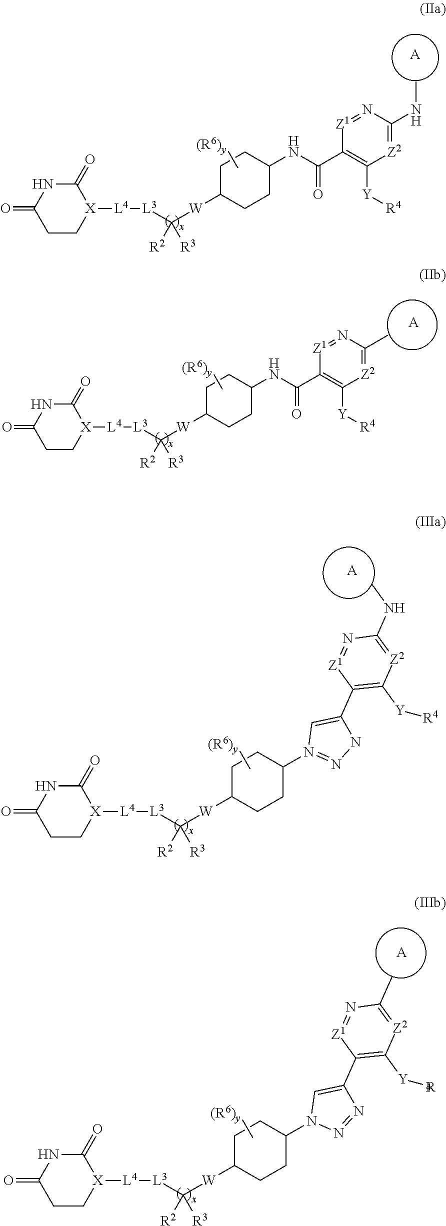

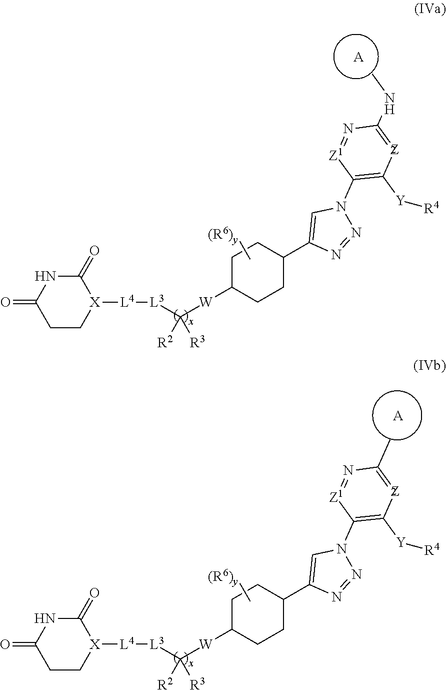

wherein Ring A, R², R³, R⁴, R⁶, L³, L⁴, W, X, Y, Z¹, Z², x, and y are as described for Formula (I′) or (I).

In some embodiments, the compound of Formula (I′) or (I) is a compound of Formula (Ia):

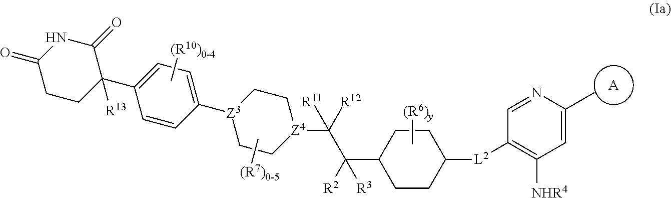

wherein Ring A, R², R³, R⁴, R⁶, R⁷, R¹⁰, R¹¹, R¹¹, R¹³, L², and y are as described for Formula (I′) or (I); and Z³ and Z⁴ are independently N or CH, provided that at least one of Z³ and Z⁴ is N. In some embodiments, Ring A is a fused bicyclic 9- to 10-membered heteroaryl containing 2-4 heteroatoms independently selected from N, O, and S, optionally substituted by 1-3 R⁰ groups, wherein R⁰ is as described for Formula (I′) or (I).

In some embodiments, the compound of Formula (I′) or (I) is a compound of Formula (Ib) or (Ic):

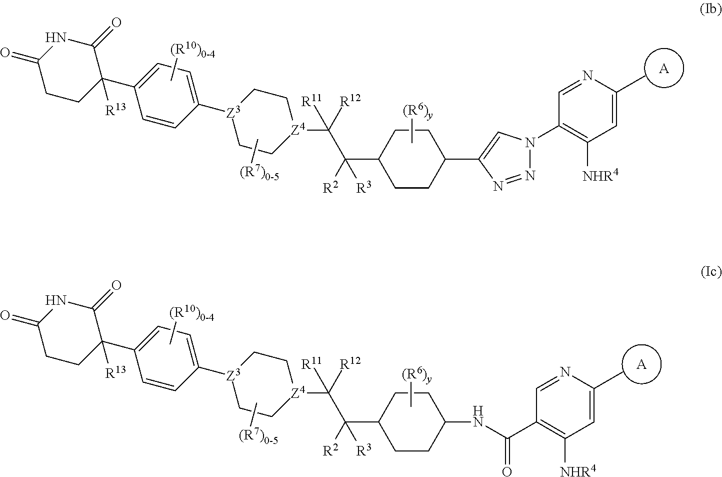

wherein Ring A, R², R³, R⁴, R⁶, R⁷, R¹⁰, R¹¹, R¹², R¹³ and y are as described for Formula (I′) or (I); and Z³ and Z⁴ are independently N or CH, provided that at least one of Z³ and Z⁴ is N.

In some embodiments, the compound of Formula (I′) or (I) is a compound of Formula (Id) or (Ie):

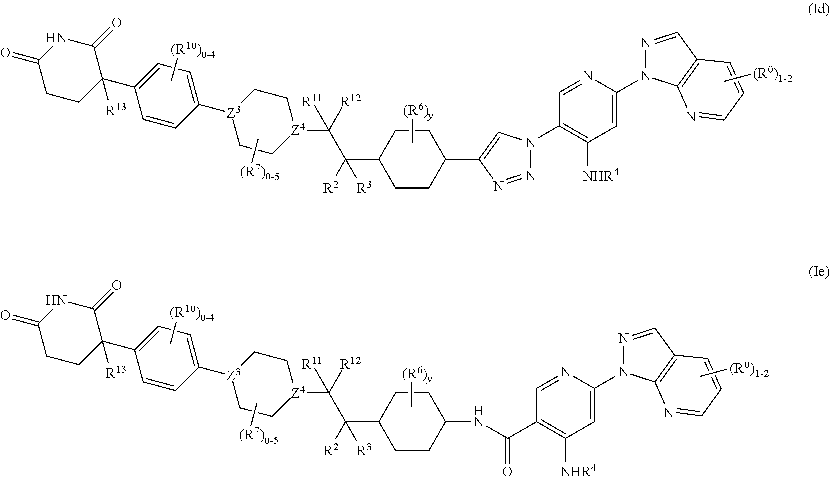

wherein R⁰, R², R³, R⁴, R⁶, R⁷, R¹⁰, R¹¹, R¹², R¹³, and y are as described for Formula (I′) or (I); and Z³ and Z⁴ are independently N or CH, provided that at least one of Z³ and Z⁴ is N.

In some embodiments, the compound of Formula (I′) or (I) is a compound of Formula (If) or (Ig):

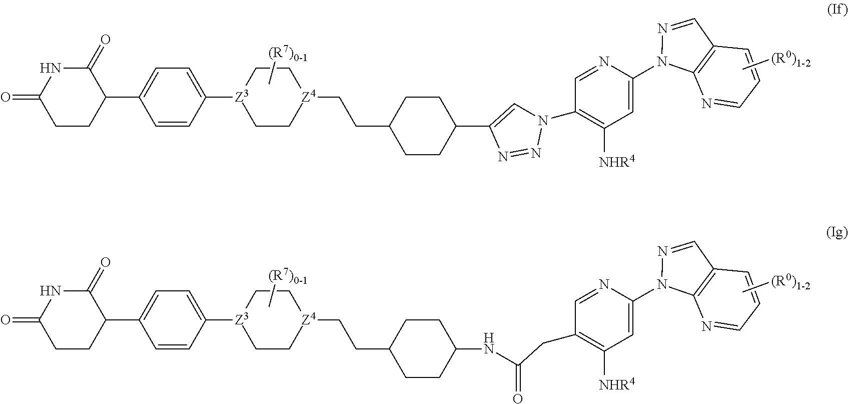

wherein R⁰, R⁴, and R⁷ are as described for Formula (I′) or (I); and Z³ and Z⁴ are independently N or CH, provided that at least one of Z³ and Z⁴ is N. In some embodiments, each R⁰ is independently —CN or —NH₂; R⁴ is C₁-C₃ alkyl-CN, or C₃-C₆ cycloalkyl optionally substituted with C₁-C₃ alkyl; and R⁷ is C₁-C₃ alkyl.

In some embodiments, the compound of Formula (I′) or (I) is a compound of Formula (Ia′):

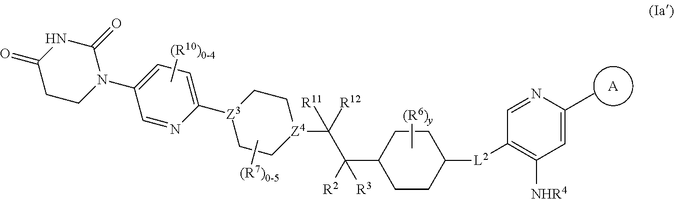

wherein Ring A, R², R³, R⁴, R⁶, R⁷, R¹⁰, R¹¹, R¹¹, L², and y are as described for Formula (I′) or (I); and Z³ and Z⁴ are independently N or CH, provided that at least one of Z³ and Z⁴ is N. In some embodiments, Ring A is a fused bicyclic 9- to 10-membered heteroaryl containing 2-4 heteroatoms independently selected from N, O, and S, optionally substituted by 1-3 R⁰ groups, wherein R⁰ is as described for Formula (I′) or (I).

In some embodiments, the compound of Formula (I′) or (I) is a compound of Formula (Ib′) or (Ic′):

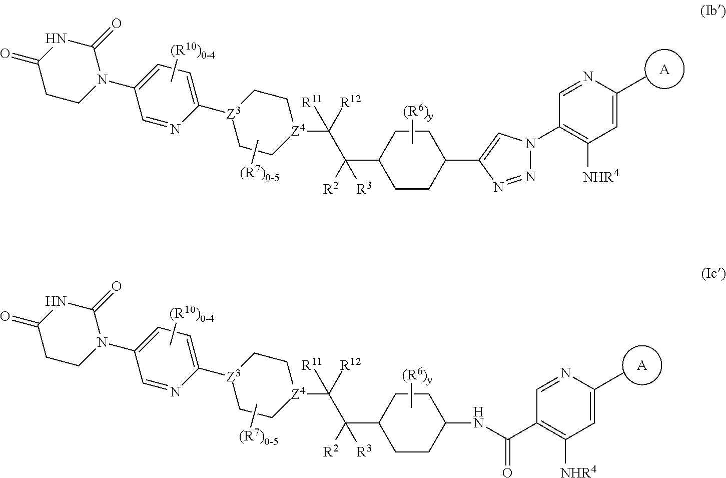

wherein Ring A, R², R³, R⁴, R⁶, R⁷, R¹⁰, R¹¹, R¹², and y are as described for Formula (I′) or (I); and Z³ and Z⁴ are independently N or CH, provided that at least one of Z³ and Z⁴ is N.

In some embodiments, the compound of Formula (I′) or (I) is a compound of Formula (Id′) or (Ie′):

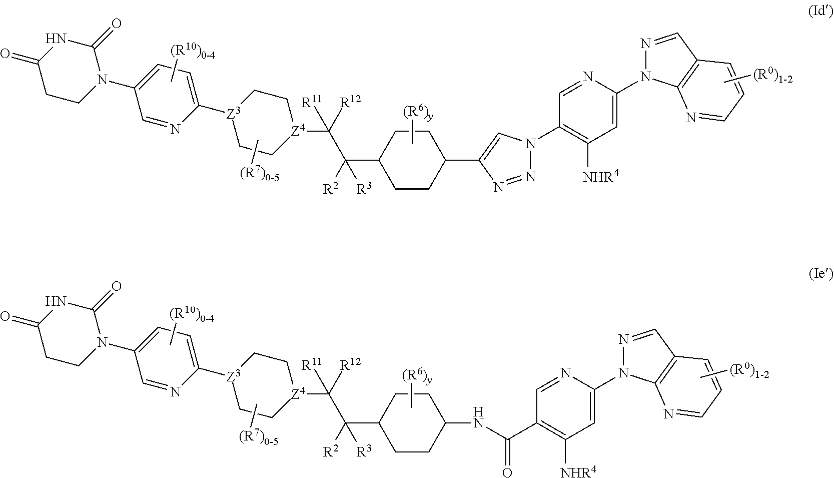

wherein R⁰, R², R³, R⁴, R⁶, R⁷, R¹⁰, R¹¹, R¹², and y are as described for Formula (I′) or (I); and Z³ and Z⁴ are independently N or CH, provided that at least one of Z³ and Z⁴ is N.

In some embodiments, the compound of Formula (I′) or (I) is a compound of Formula (If′) or (Ig′):

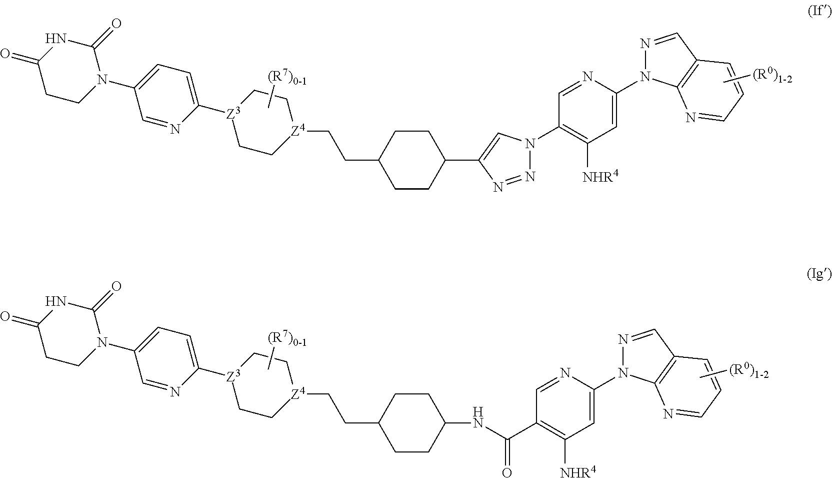

wherein R⁰, R⁴, and R⁷ are as described for Formula (I′) or (I); and Z³ and Z⁴ are independently N or CH, provided that at least one of Z³ and Z⁴ is N. In some embodiments, each R⁰ is independently —CN or —NH₂; R⁴ is C₁-C₃ alkyl-CN, or C₃-C₆ cycloalkyl optionally substituted with C₁-C₃ alkyl; and R⁷ is C₁-C₃ alkyl.

In some embodiments, the compound of Formula (I′) or (I) is a compound of Formula (Va) or (Vb):

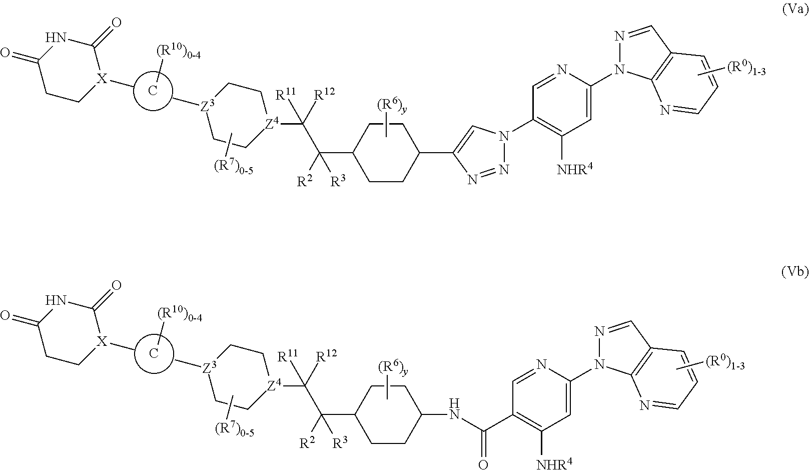

wherein R⁰, R², R³, R⁴, R⁶, R⁷, R¹⁰, R¹¹, R¹¹, R¹³, X, and y are as described for Formula (I′) or (I); Ring C is phenylene or pyridylene; and Z³ and Z⁴ are independently N or CH, provided that at least one of Z³ and Z⁴ is N.

In the descriptions herein, it is understood that every description, variation, embodiment, or aspect of a moiety may be combined with every description, variation, embodiment, or aspect of other moieties the same as if each and every combination of descriptions is specifically and individually listed. For example, every description, variation, embodiment, or aspect provided herein with respect to Ring A of Formula (I′) or (I) may be combined with every description, variation, embodiment, or aspect of L¹, L², L³, L⁴, R⁰, R^{1a}, R^{1b}, R², R³, R⁴, R⁵, R⁶, R⁷, R⁸, R⁹, R¹⁰, RH, R¹², R¹³, W, X, Y, Z¹, Z², x, and y the same as if each and every combination were specifically and individually listed. It is also understood that all descriptions, variations, embodiments, or aspects of Formula (I′) or (I), where applicable, apply equally to other formulae detailed herein, and are equally described, the same as if each and every description, variation, embodiment, or aspect were separately and individually listed for all formulae. For example, all descriptions, variations, embodiments, or aspects of Formula (I′) or (I), where applicable, apply equally to any of the formulae as detailed herein, such as Formulae (IA), (IB), (IC), (ID), (IE), (Ia), (Ib), (Ic), (Id), (Ie), (If), (Ig), (Ia′), (Ib′), (Ic′), (Id′), (Ie′), (If′), (Ig′), (IIa), (IIb), (IIIa), (IIIb), (IVa), (IVb), (Va), and (Vb) are equally described, the same as if each and every description, variation, embodiment, or aspect were separately and individually listed for all formulae.

In some embodiments, provided is a compound selected from the compounds in Table 1 or a pharmaceutically acceptable salt thereof. Although certain compounds described in the present disclosure, including in Table 1, are presented as specific stereoisomers and/or in a non-stereochemical form, it is understood that any or all stereochemical forms, including any enantiomeric or diastereomeric forms, and any tautomers or other forms of any of the compounds of the present disclosure, including in Table 1, are herein described.

TABLE 1

| Compound | |
|---|---|
| No. | Structure |
| P-1 | 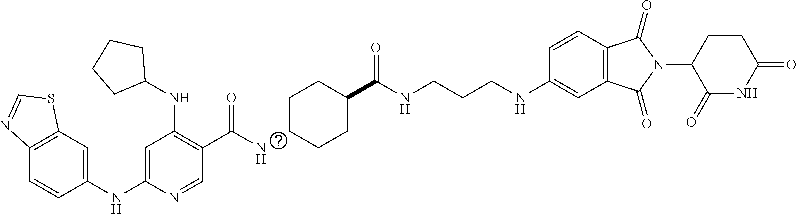 |
| P-2 | 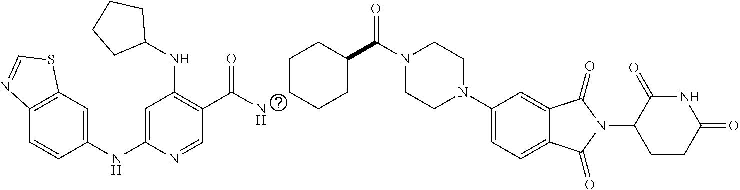 |
| P-3 | 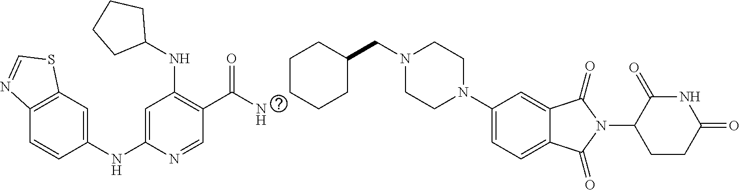 |
| P-4 | 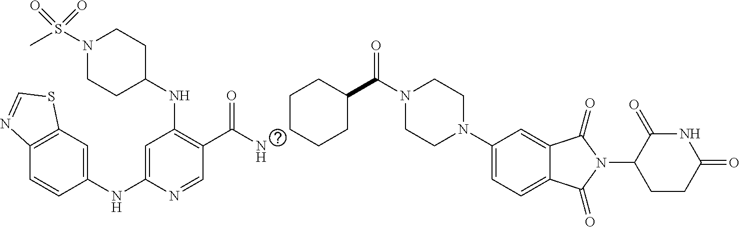 |
| P-5 | 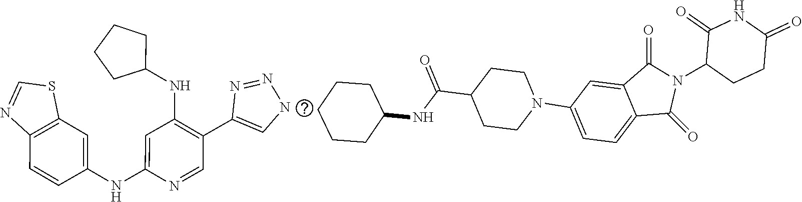 |
| P-6 | 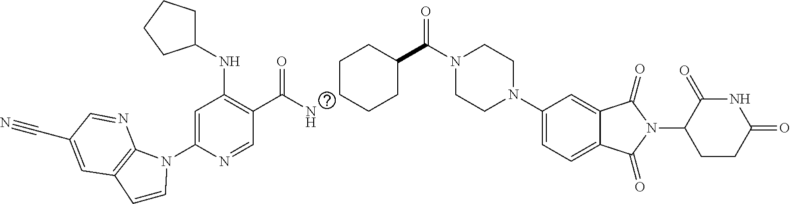 |
| P-7 | 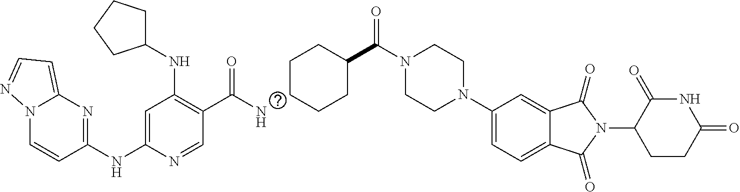 |
| P-8 | 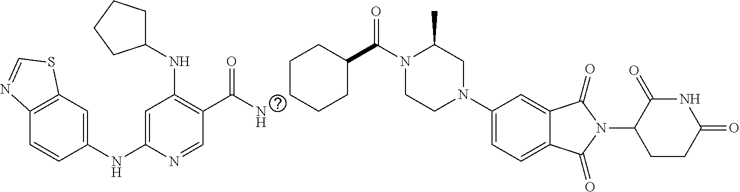 |
| P-9 | 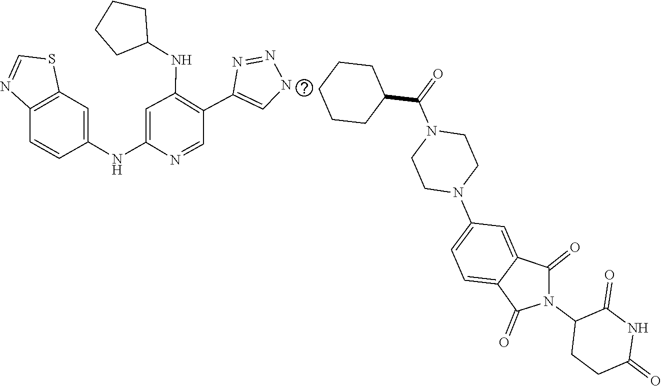 |
| P-10 | 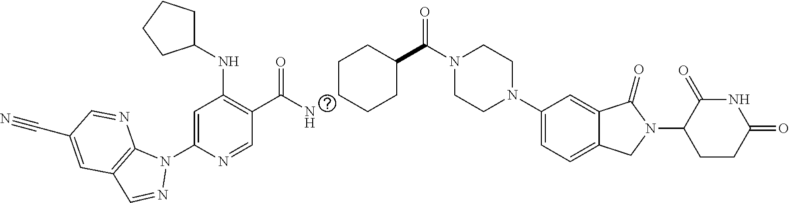 |
| P-11 | 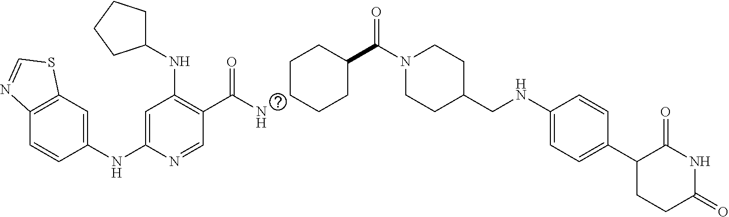 |
| P-12 | 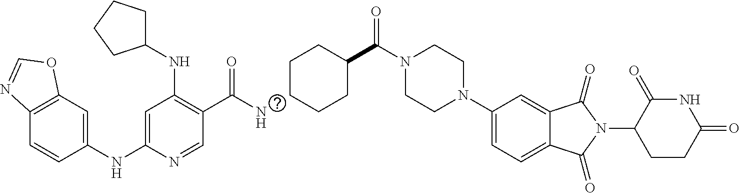 |
| P-13 | 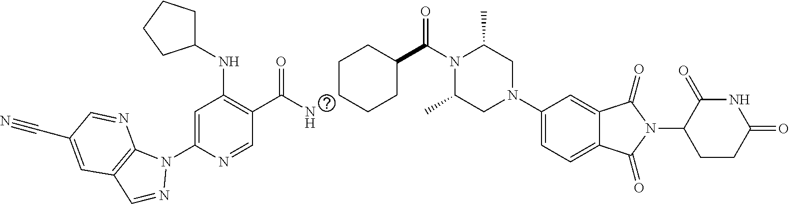 |
| P-14 | 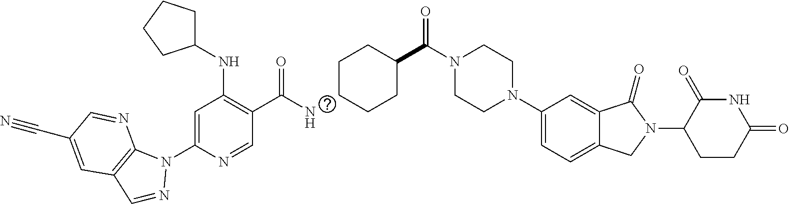 |
| P-15 | 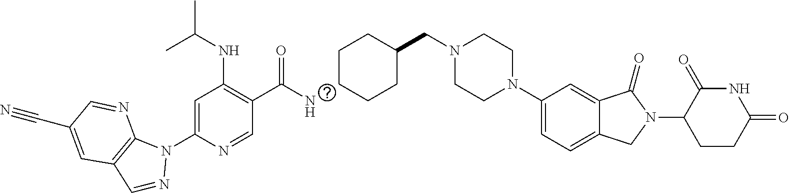 |
| P-16 | 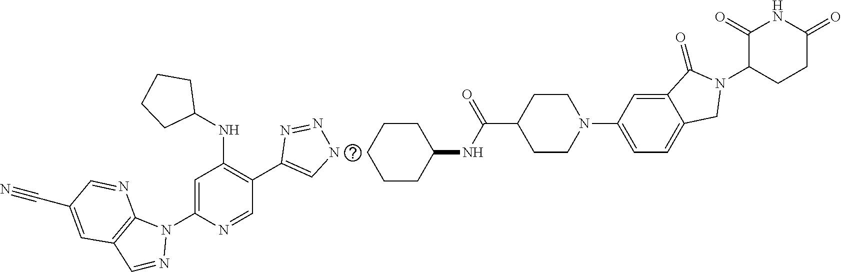 |
| P-17 | 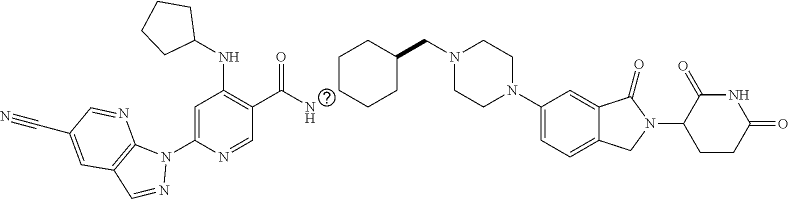 |
| P-18 | 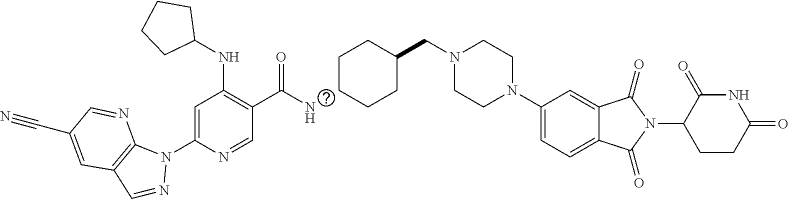 |
| P-19 | 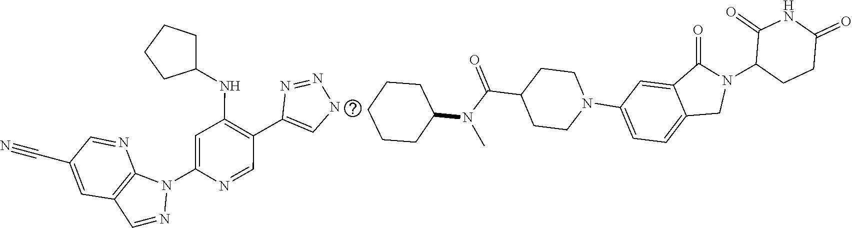 |
| P-20 | 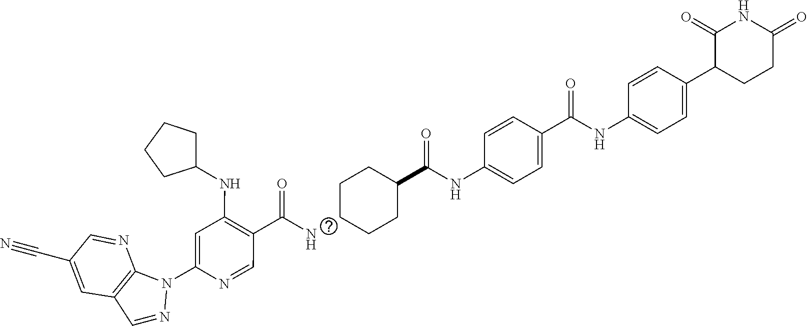 |
| P-21 | 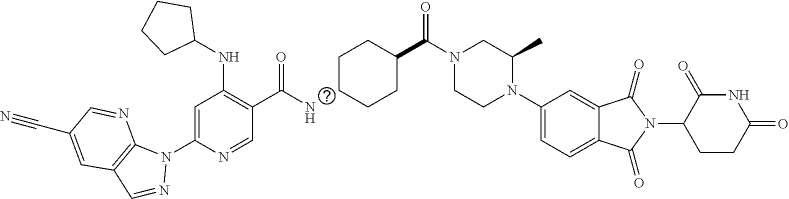 |
| P-22 | 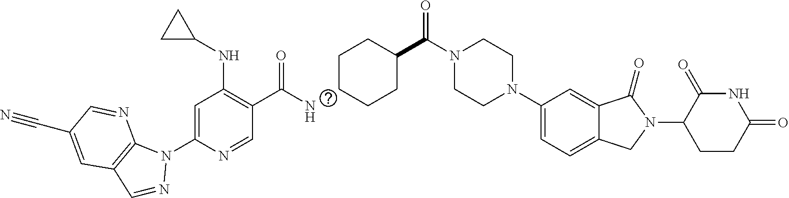 |
| P-23 | 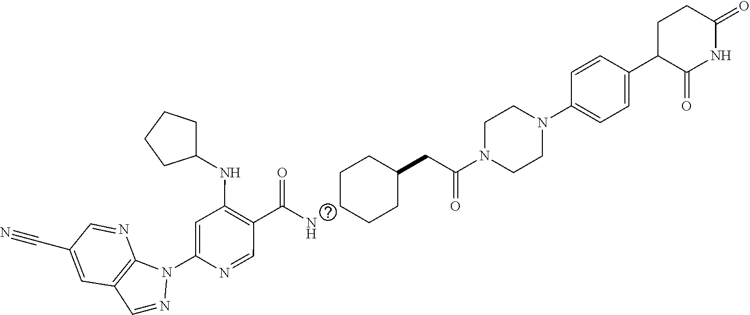 |
| P-24 | 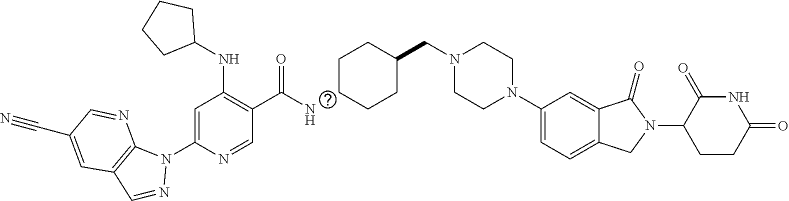 |
| P-25 | 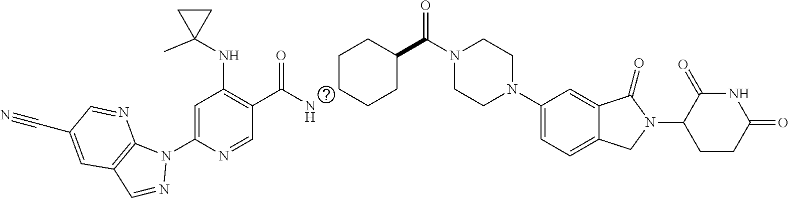 |
| P-26 | 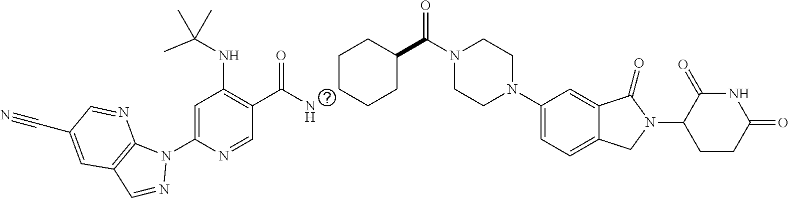 |
| P-27 | 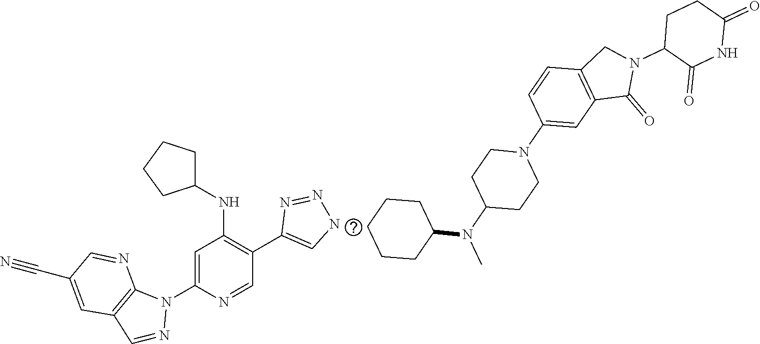 |
| P-28 | 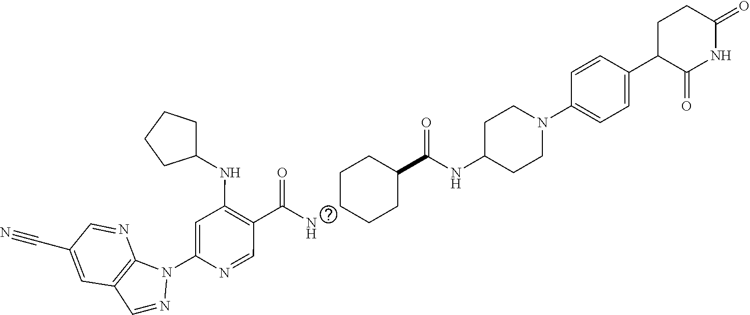 |
| P-29 | 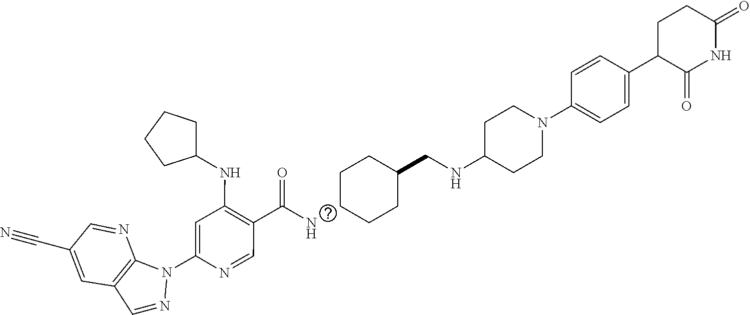 |
| P-30 | 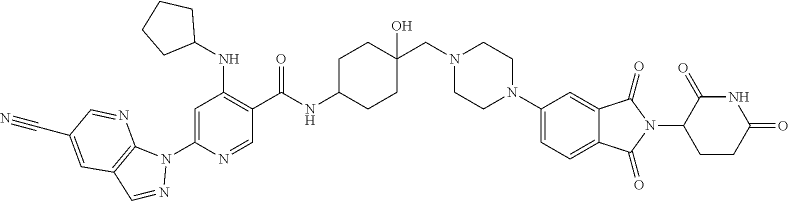 [SMILES: N#Cc1cnc2c(cnn2-c2cc(NC3CCCC3)c(C(=O)NC3CCC(O)(CN4CCN(c5ccc6c(c5)C(=O)N(C5CCC(=O)NC5=O)C6=O)CC4)CC3)cn2)c1] |
| P-31 | 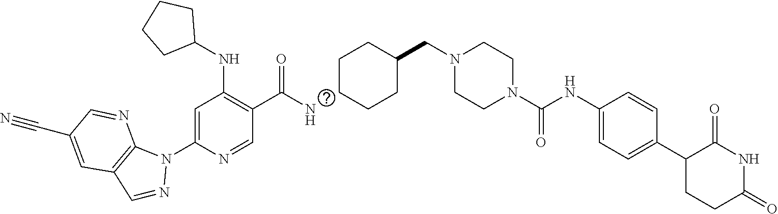 |
| P-32 | 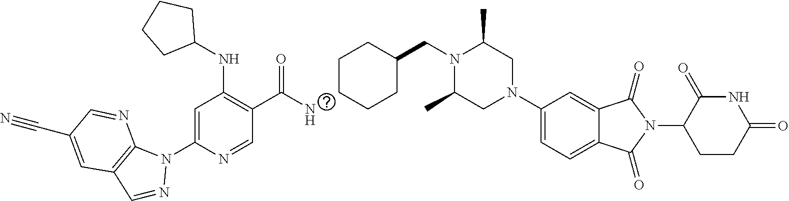 |
| P-33 | 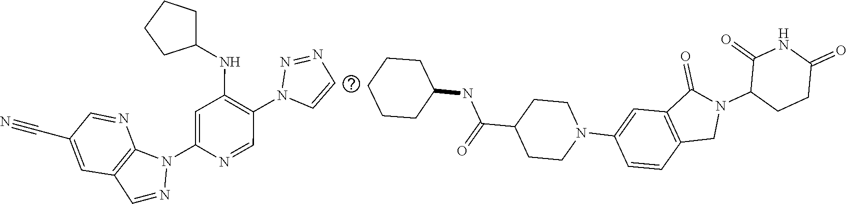 |
| P-34 | 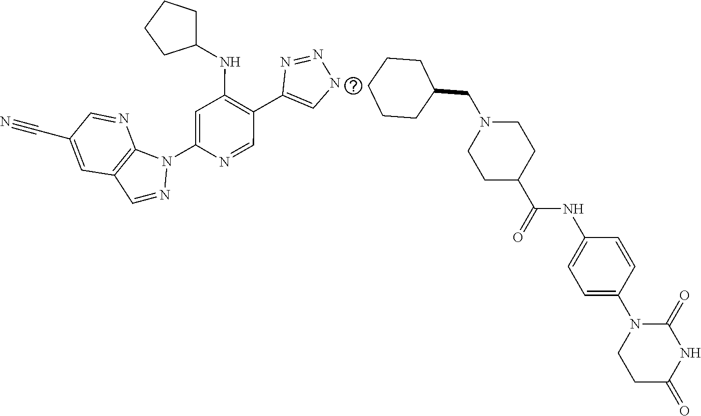 |
| P-35 | 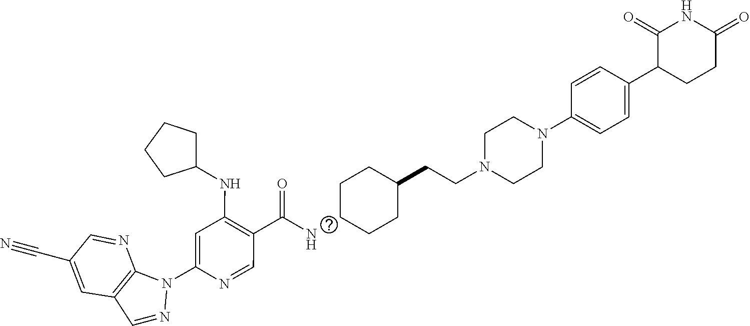 |
| P-36 | 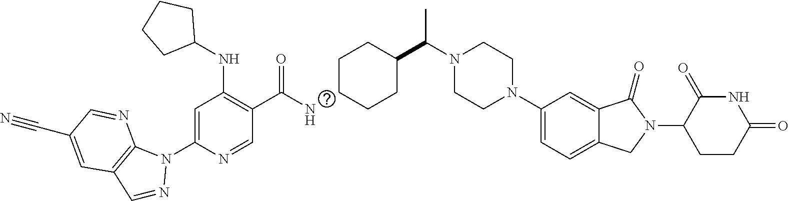 |
| P-37 | 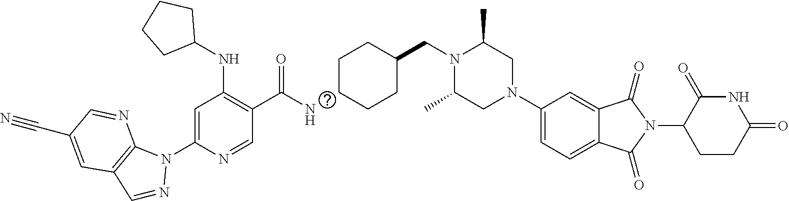 |
| P-38 | 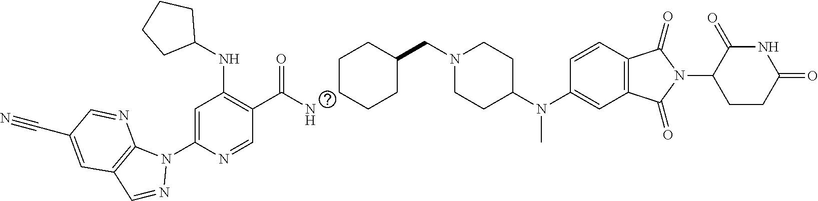 |
| P-39 | 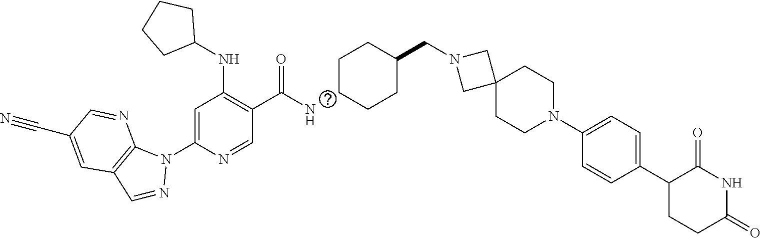 |
| P-40 | 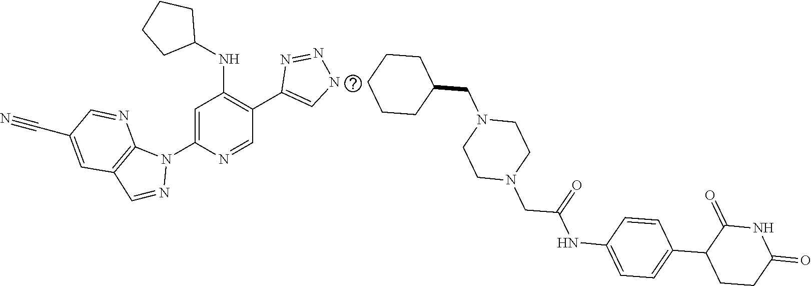 |
| P-41 | 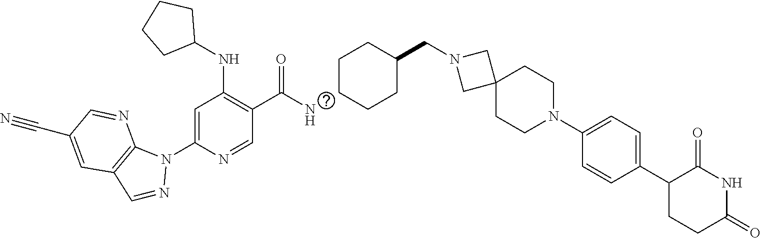 |
| P-42 | 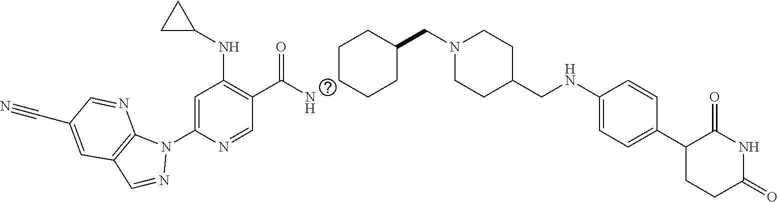 |
| P-43 | 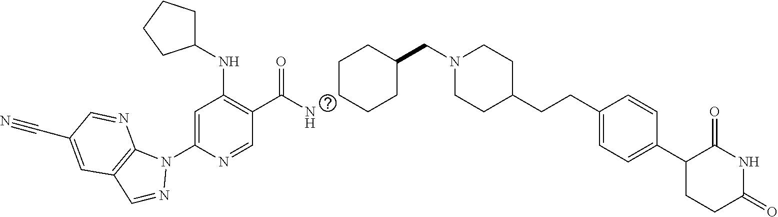 |
| P-44 | 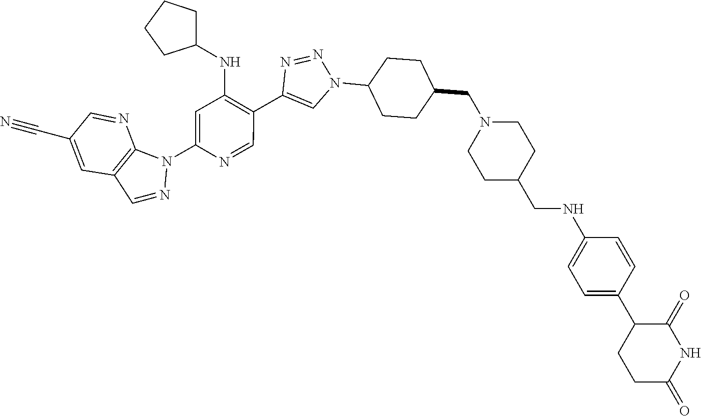 |
| P-45 | 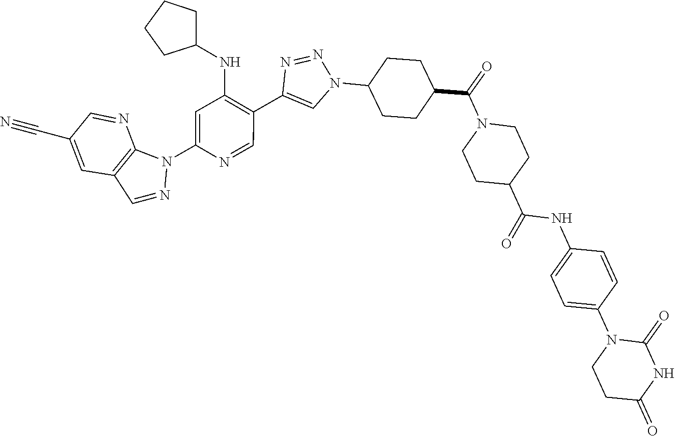 |
| P-46 | 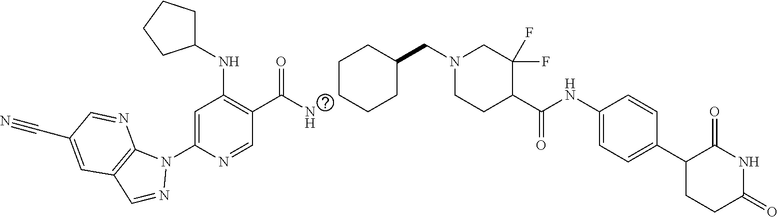 |
| P-47 | 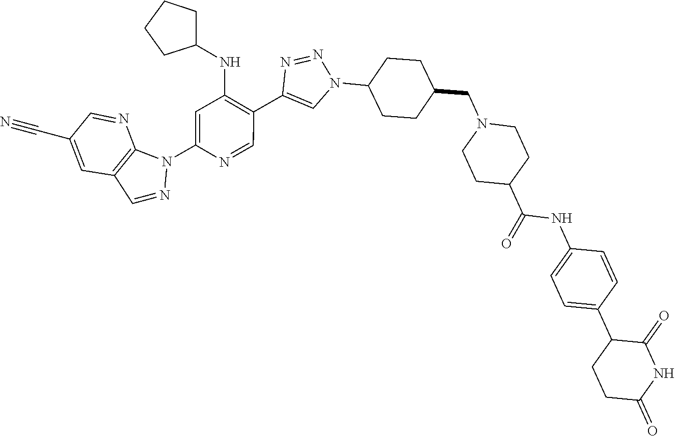 |
| P-48 | 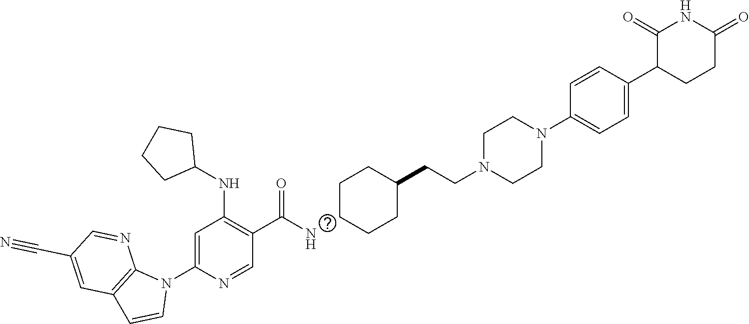 |
| P-49 | 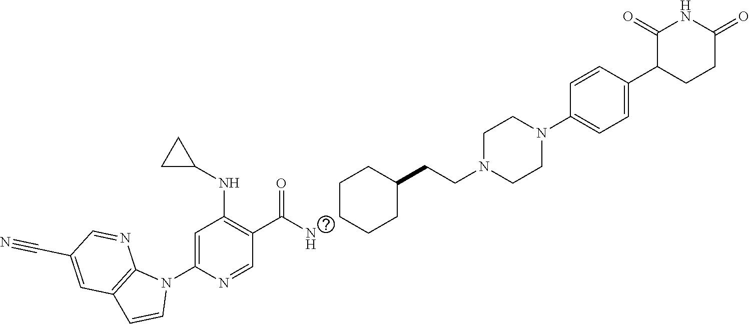 |
| P-50 | 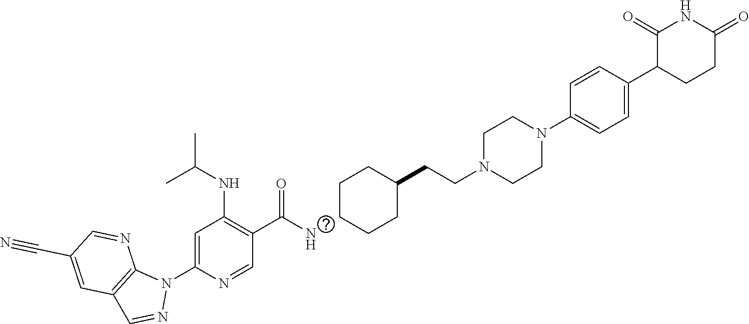 |
| P-51 | 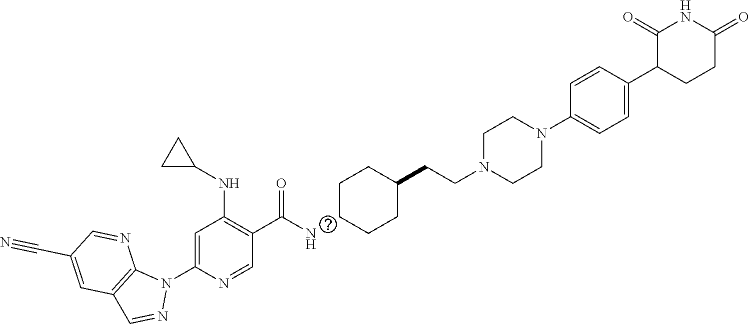 |
| P-52 | 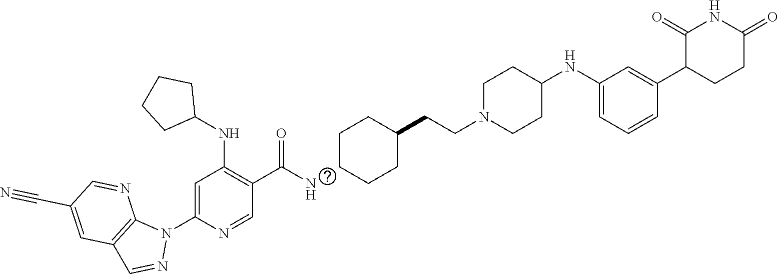 |
| P-53 | 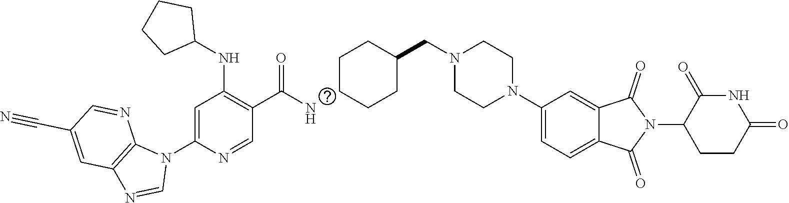 |
| P-54 | 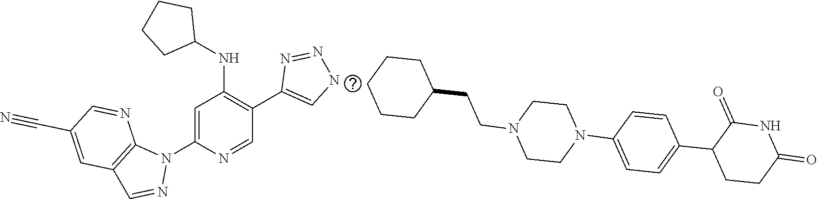 |
| P-55 | 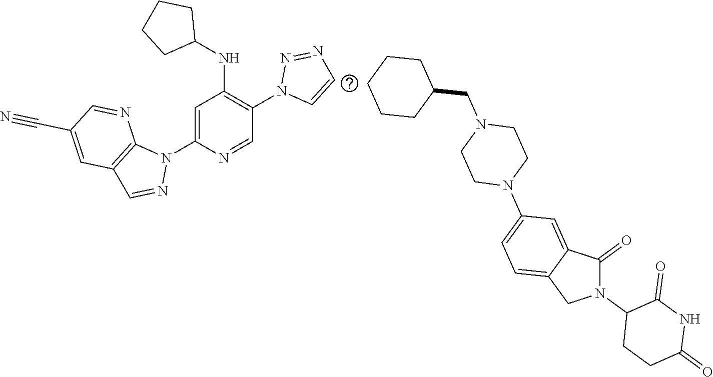 |
| P-56 | 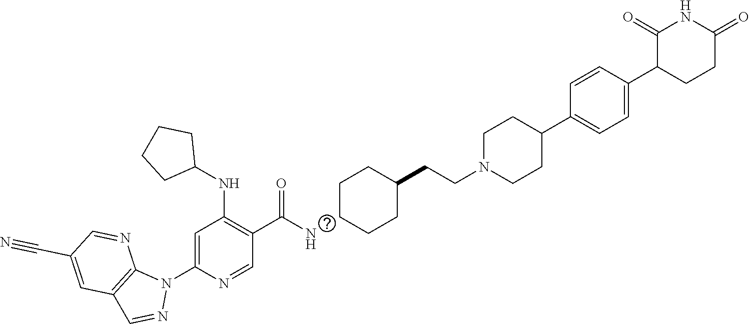 |
| P-57 | 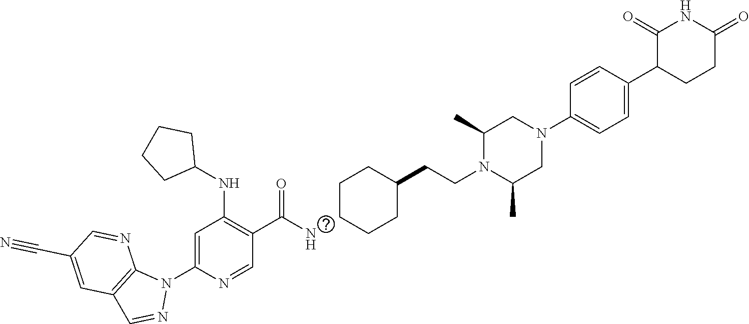 |
| P-58 | 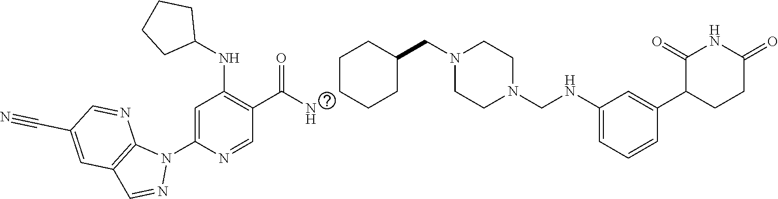 |
| P-59 | 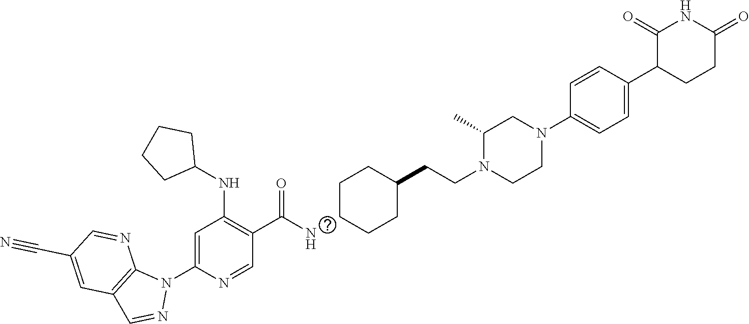 |
| P-60 | 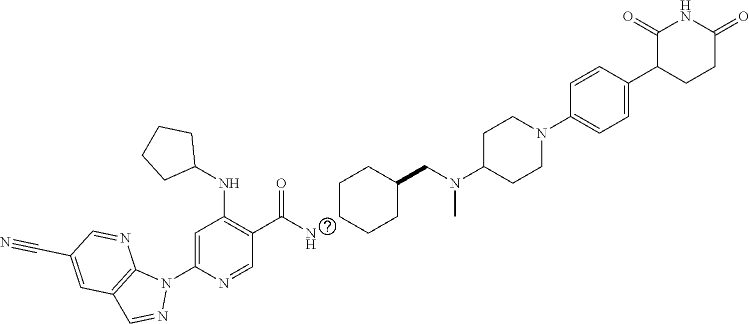 |
| P-61 | 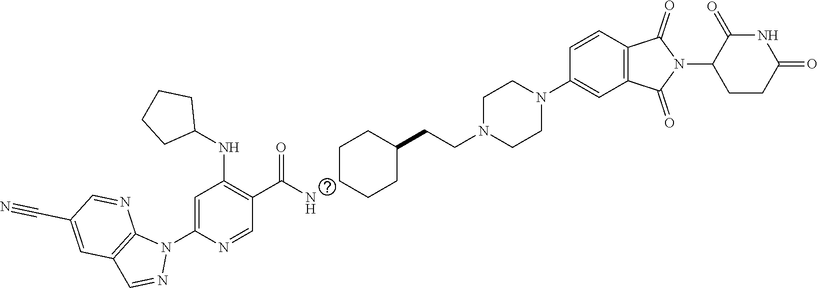 |
| P-62 | 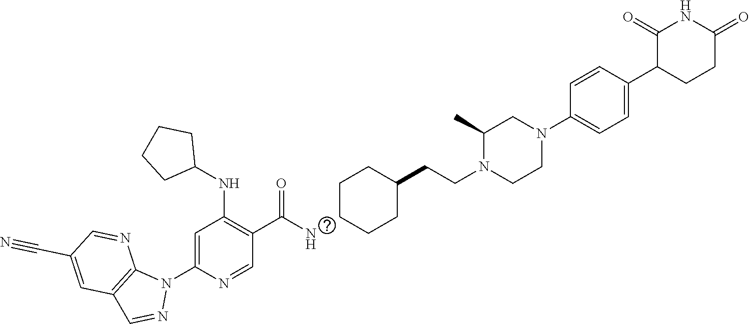 |
| P-63 | 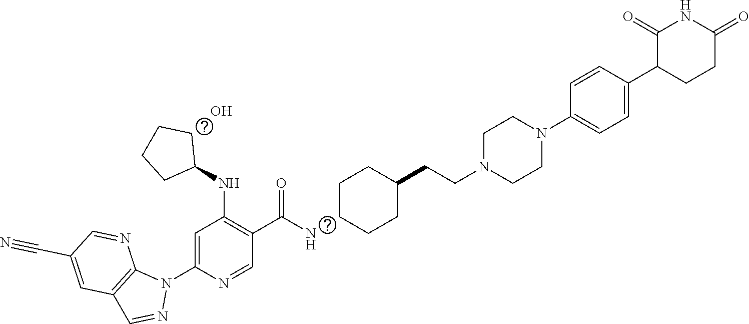 |
| P-64 | 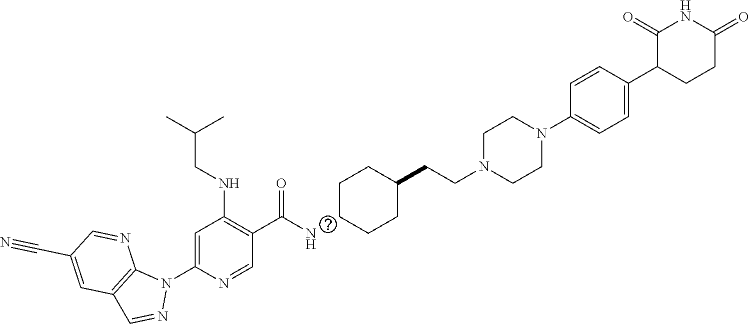 |
| P-65 | 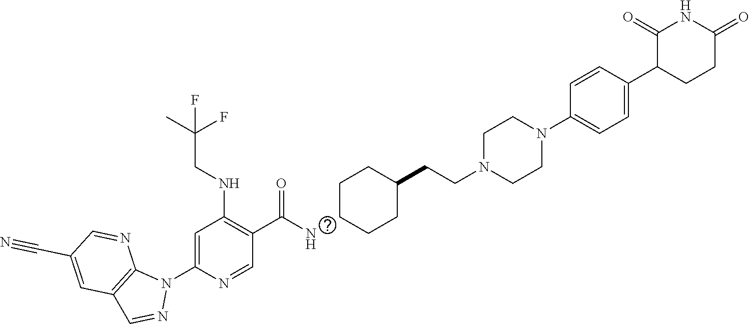 |
| P-66 | 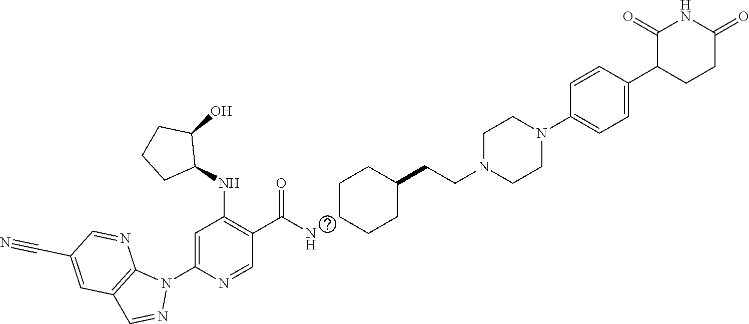 |
| P-67 | 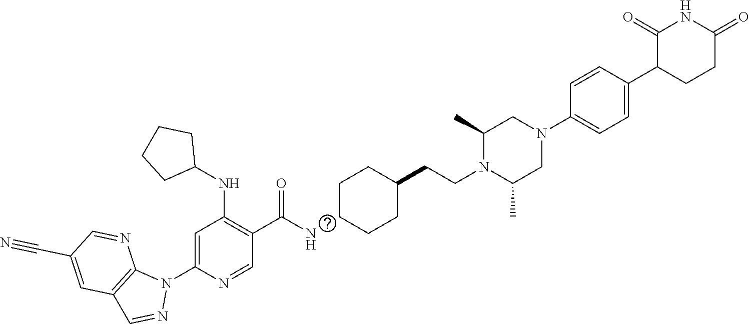 |
| P-68 | 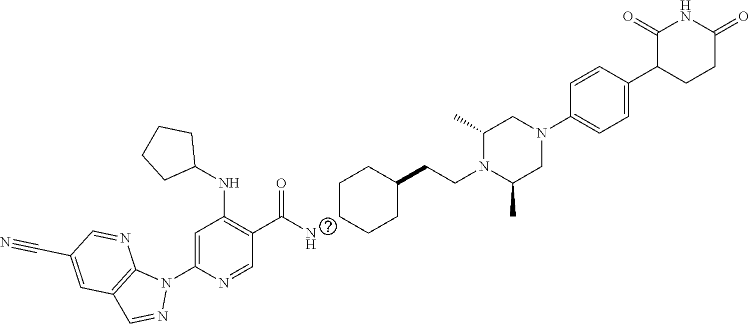 |
| P-69 | 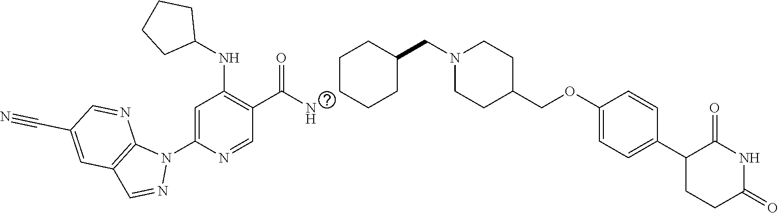 |
| P-70 | 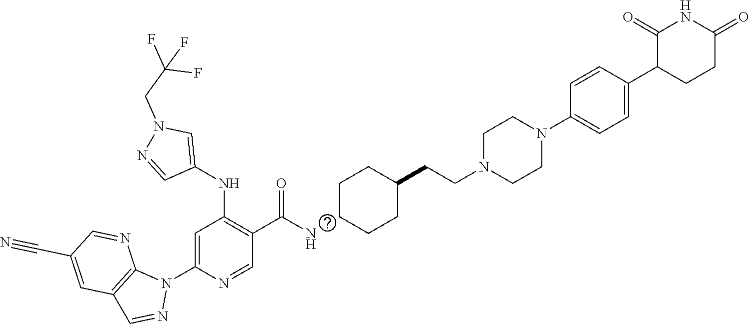 |
| P-71 | 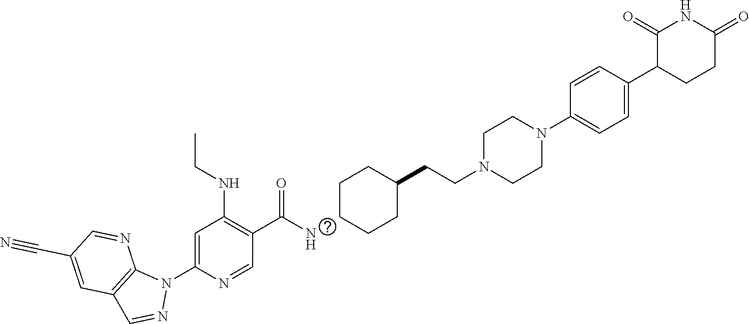 |
| P-72 | 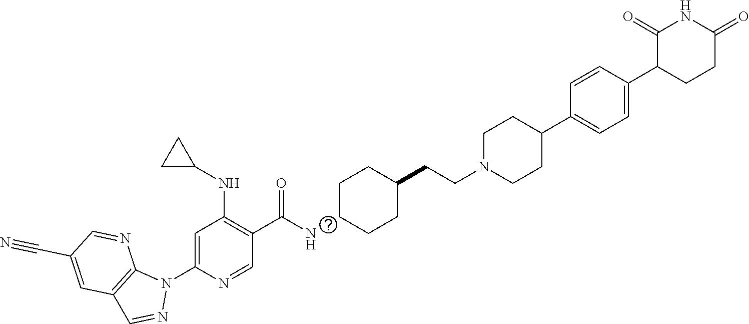 |
| P-73 | 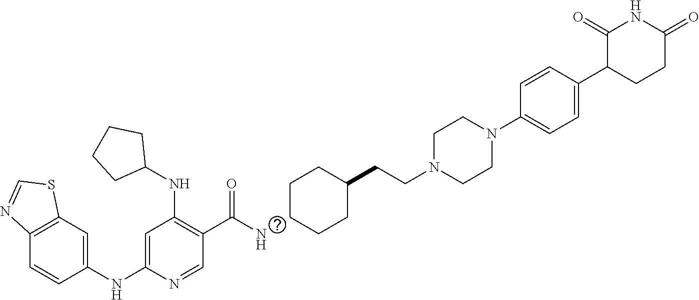 |
| P-74 | 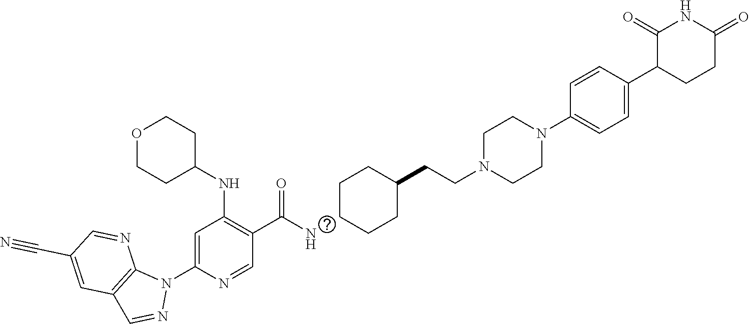 |
| P-75 | 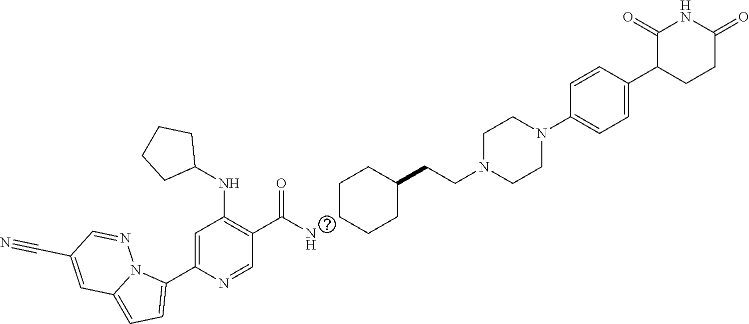 |
| P-76 | 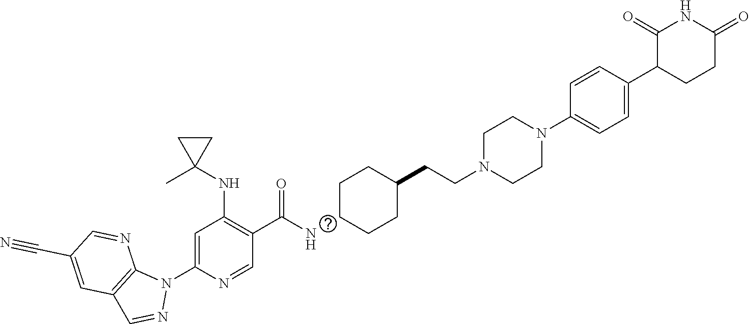 |
| P-77 | 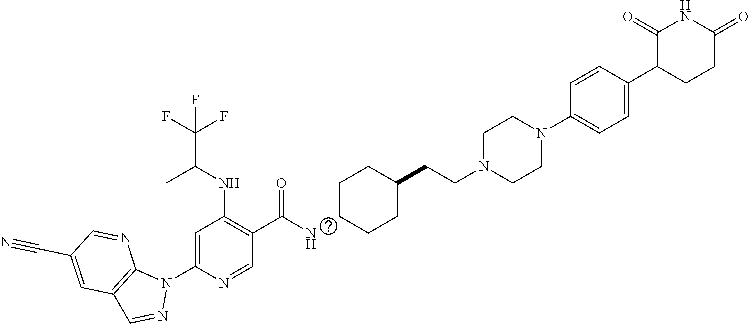 |
| P-78 | 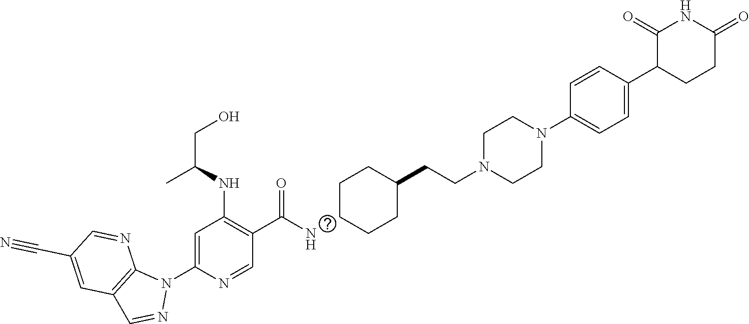 |
| P-79 | 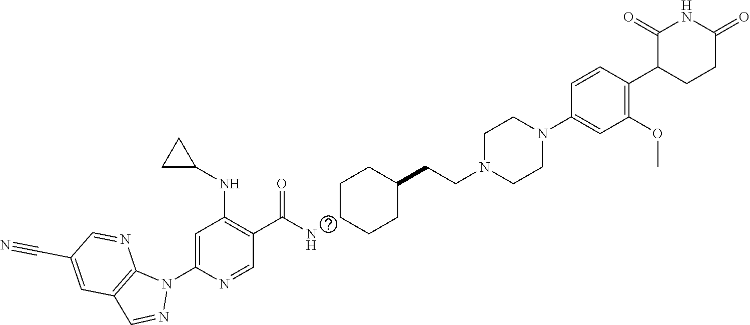 |
| P-80 | 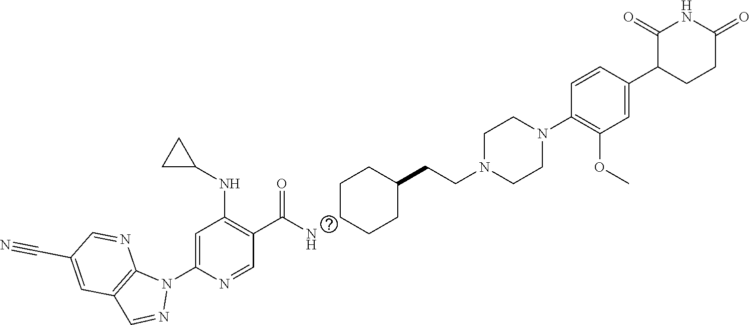 |
| P-81 | 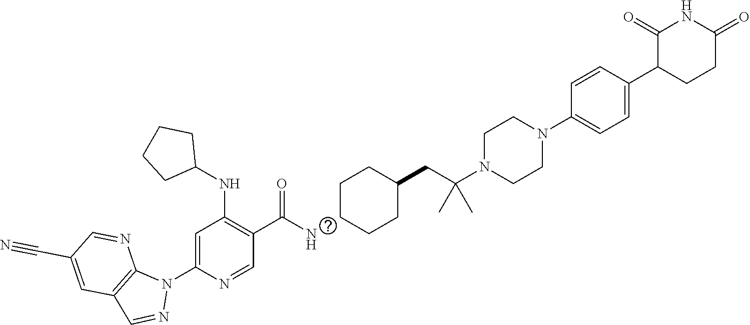 |
| P-82 | 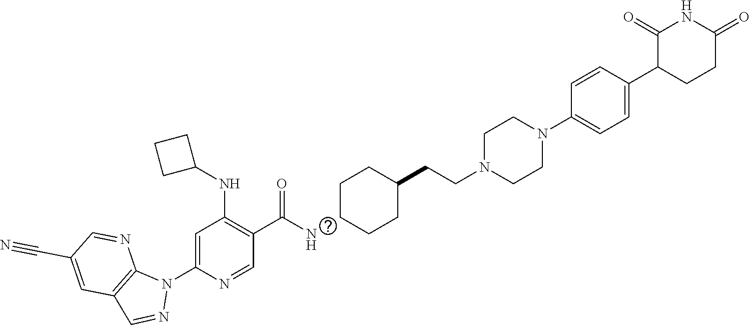 |
| P-83 | 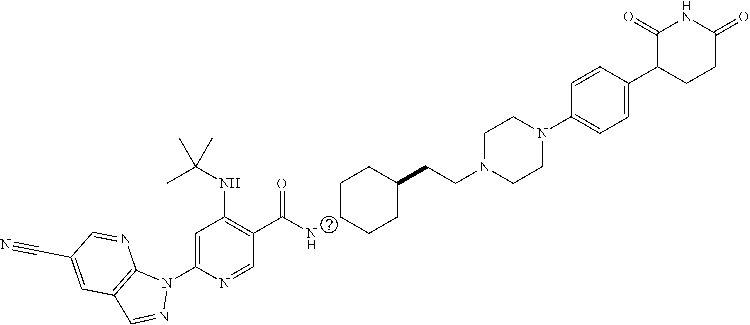 |
| P-84 | 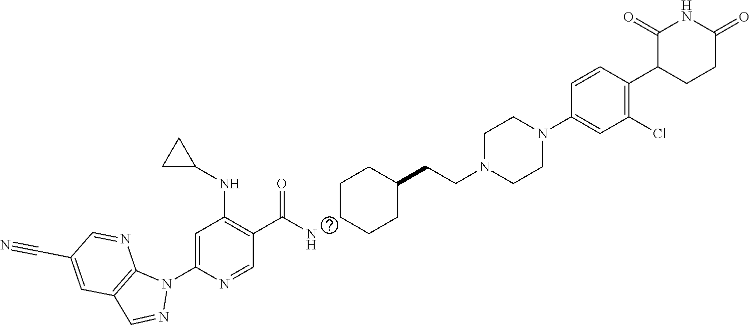 |
| P-85 | 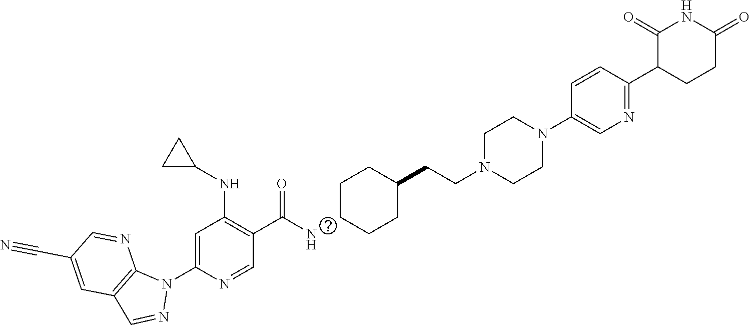 |
| P-86 | 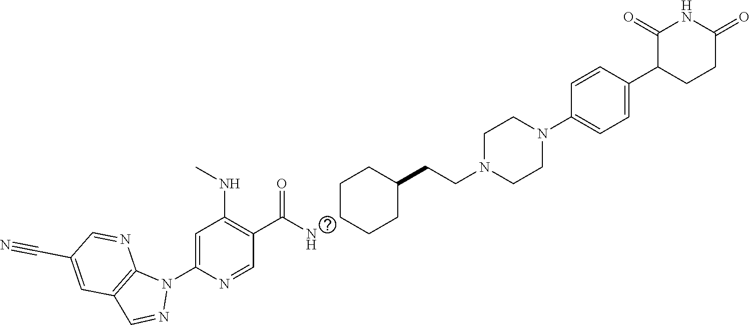 |
| P-87 | 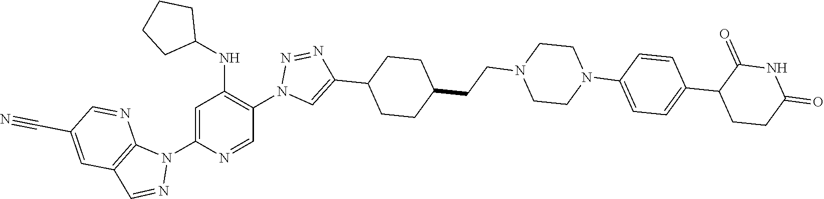 |
| P-88 | 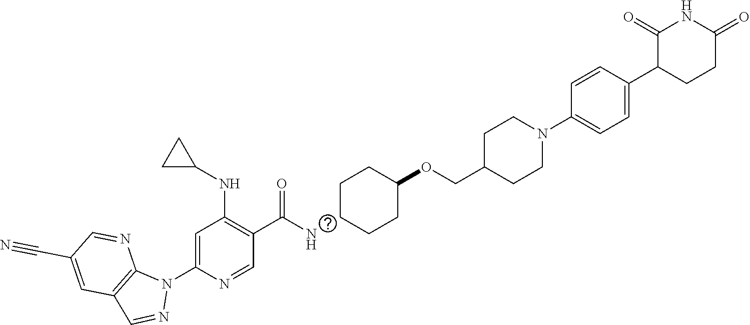 |
| P-89 | 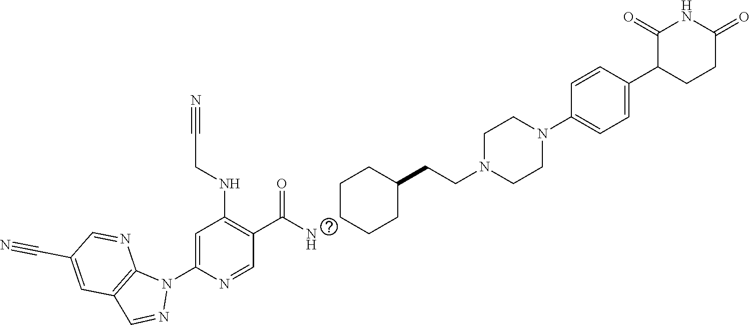 |
| P-90 | 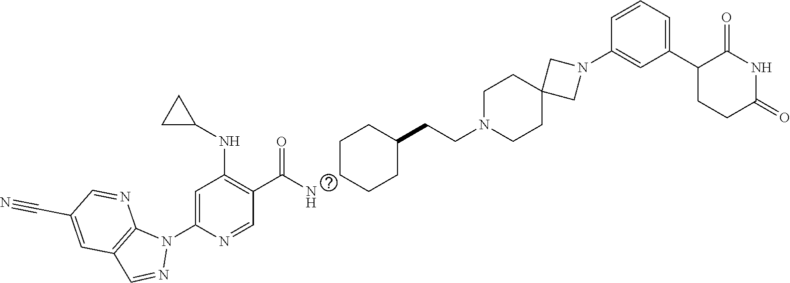 |
| P-91 | 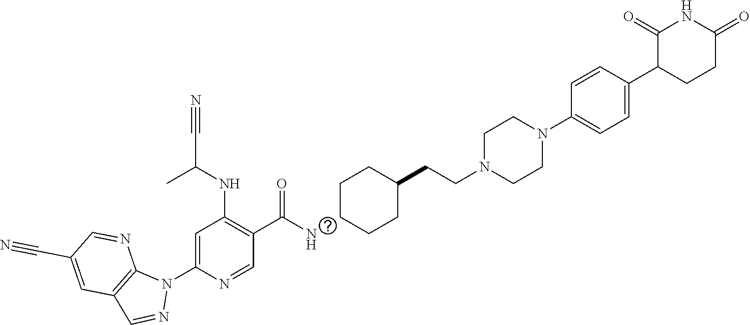 |
| P-92 | 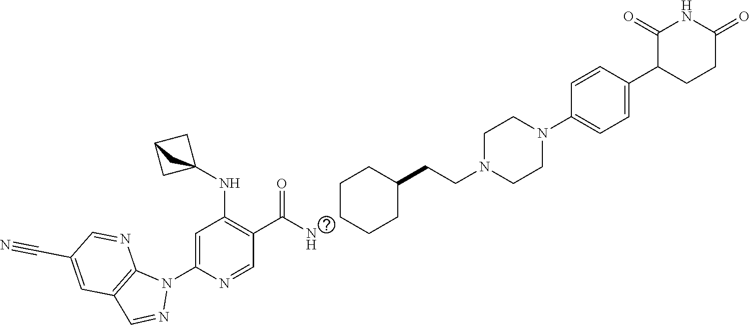 |
| P-93 | 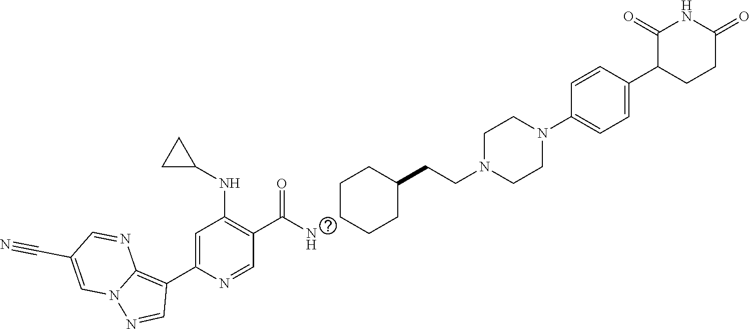 |
| P-94 | 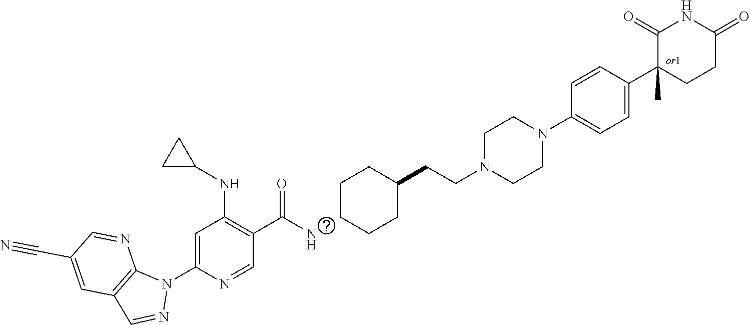 |
| P-95 | 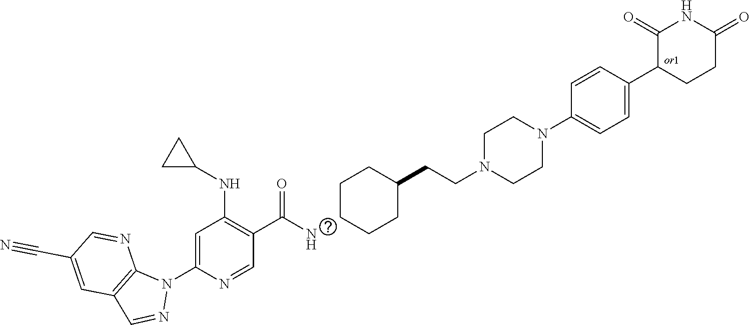 |
| P-96 | 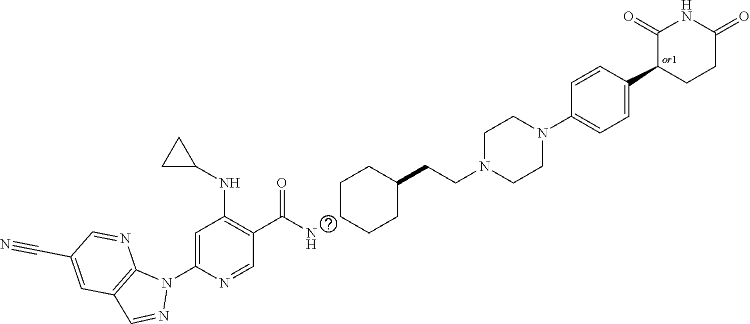 |
| P-97 | 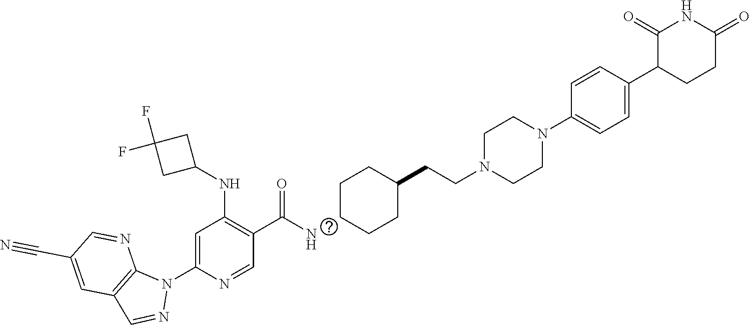 |
| P-98 | 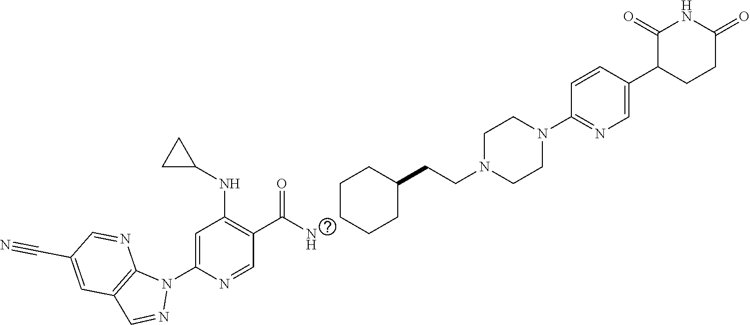 |
| P-99 | 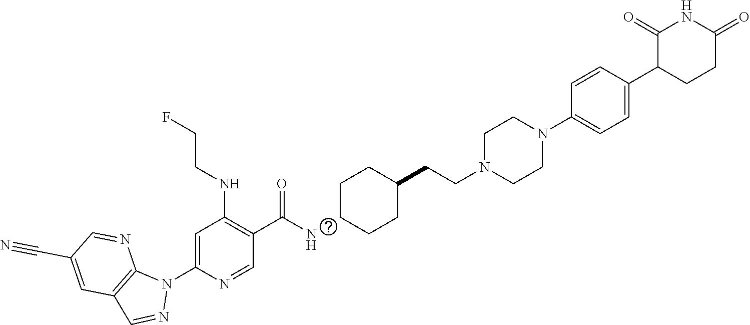 |
| P-100 | 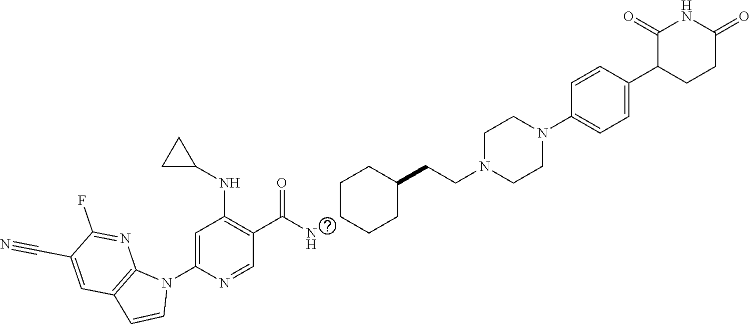 |
| P-101 | 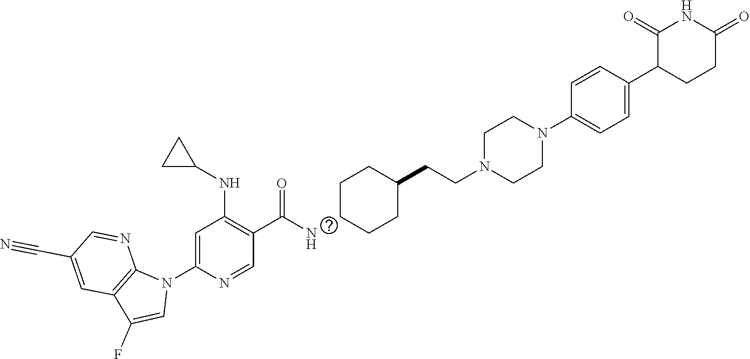 |
| P-102 | 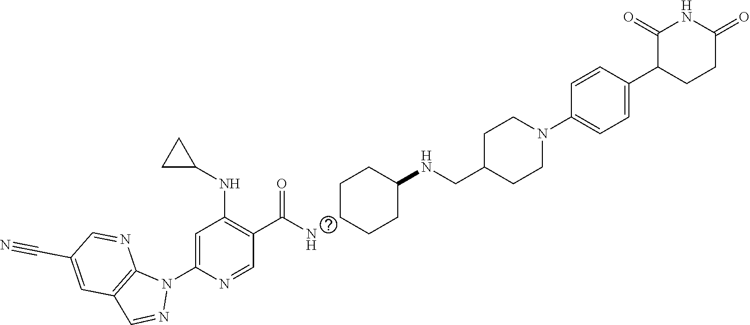 |
| P-103 | 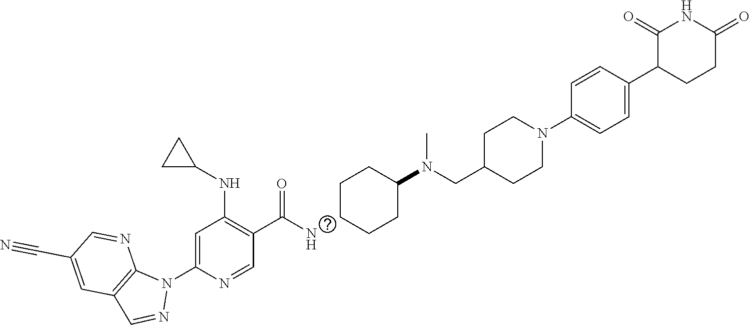 |
| P-104 | 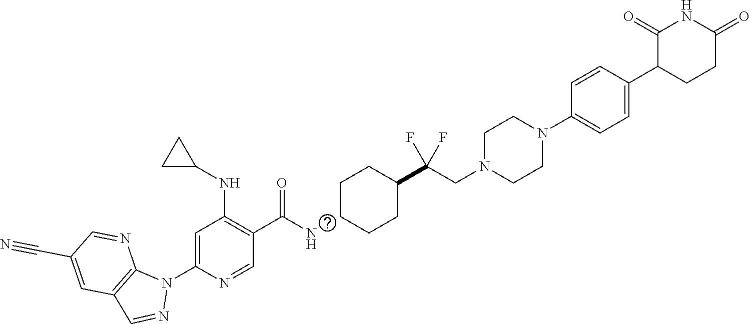 |
| P-105 | 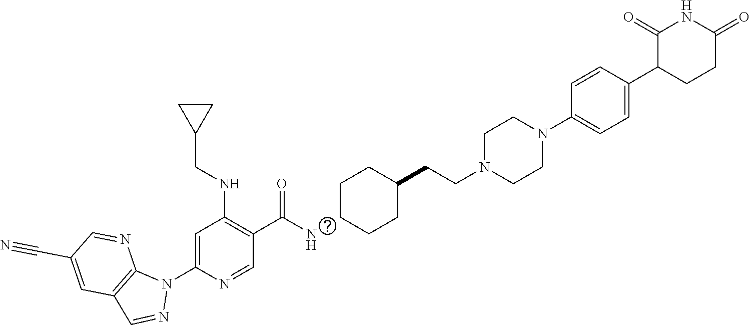 |
| P-106 | 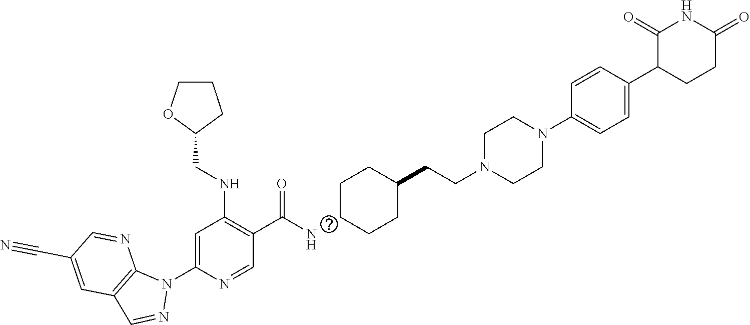 |
| P-107 | 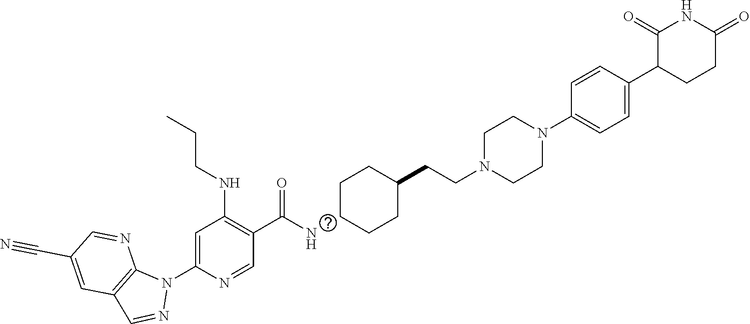 |
| P-108 | 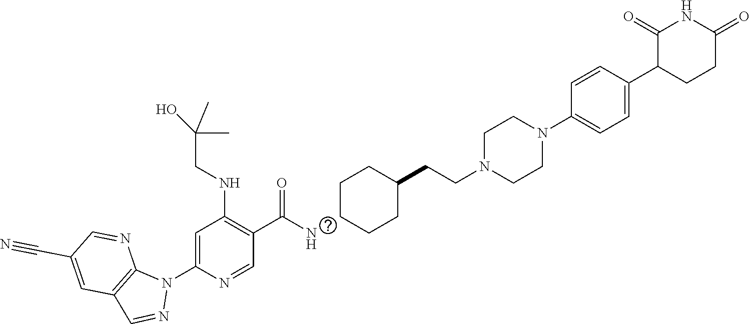 |
| P-109 | 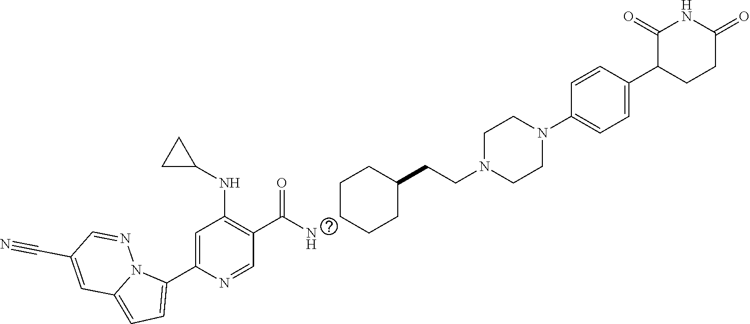 |
| P-110 | 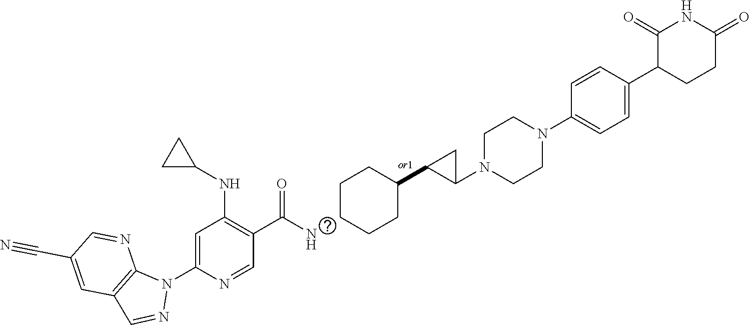 |
| P-111 | 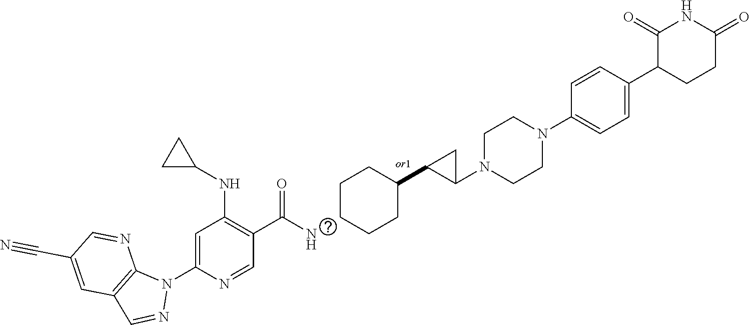 |
| P-112 | 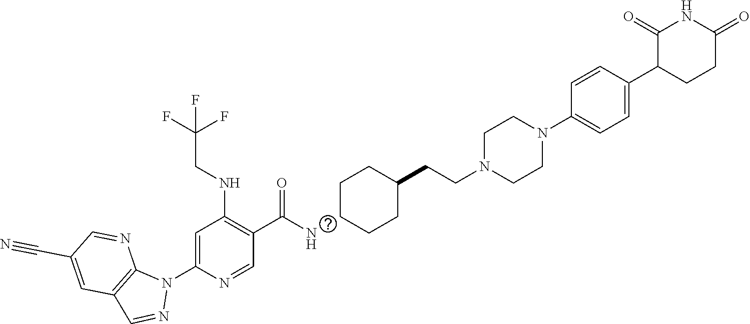 |
| P-113 | 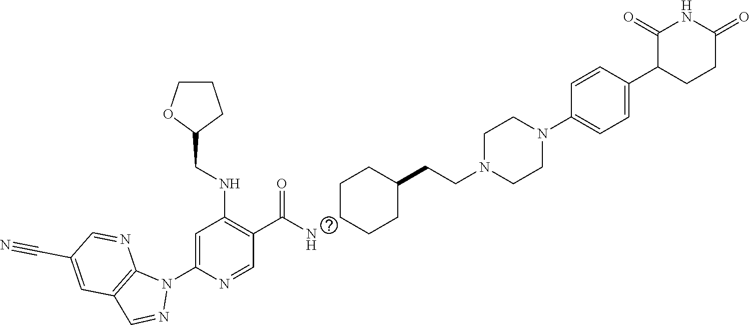 |
| P-114 | 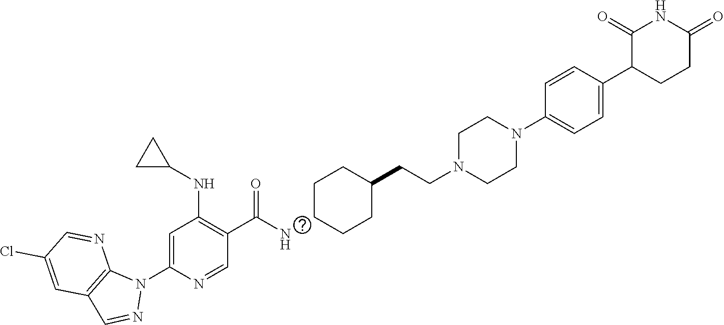 |
| P-115 | 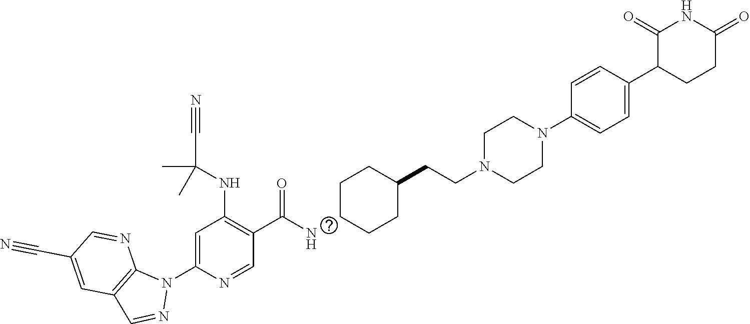 |
| P-116 | 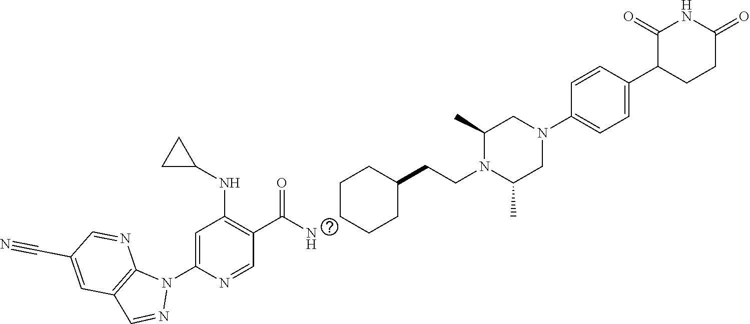 |
| P-117 | 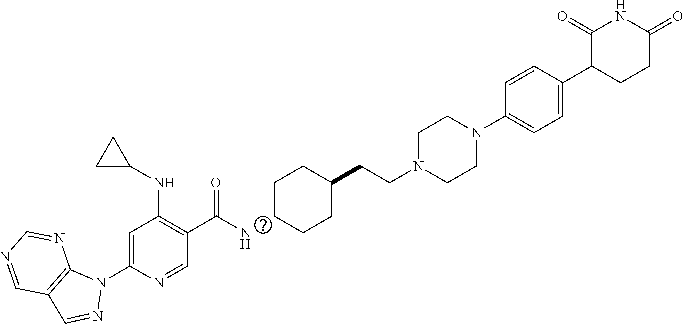 |
| P-118 | 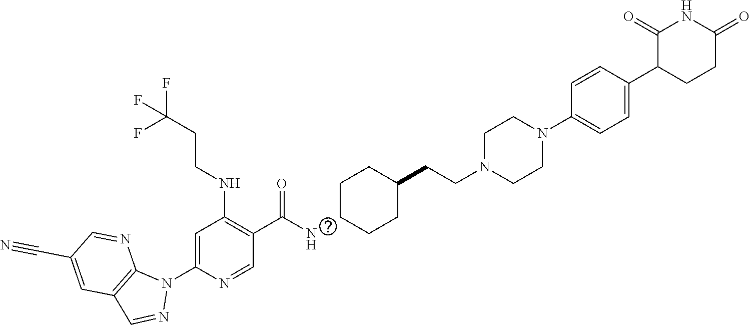 |
| P-119 | 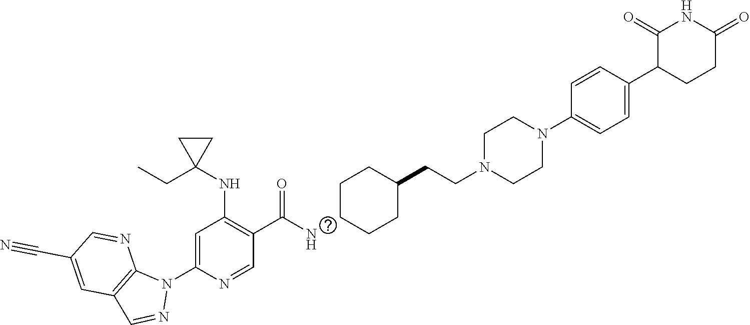 |
| P-120 | 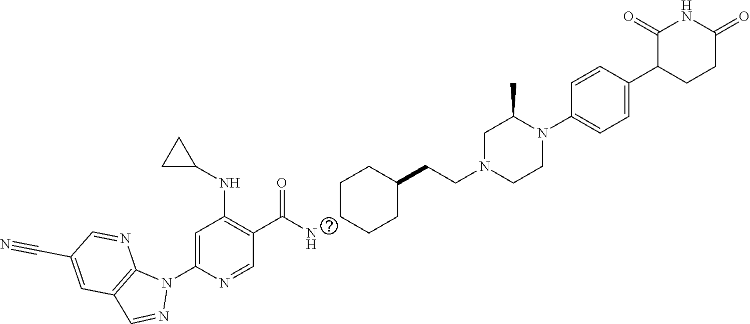 |
| P-121 | 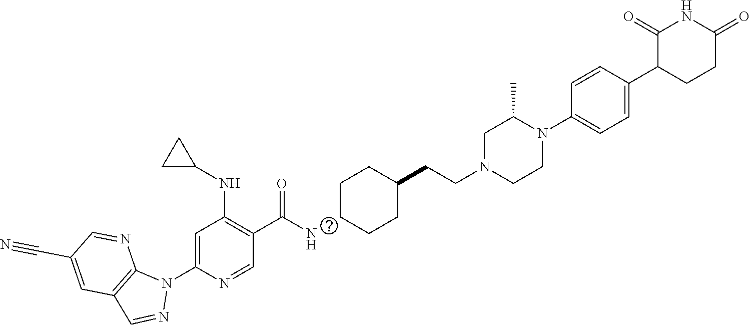 |
| P-122 | 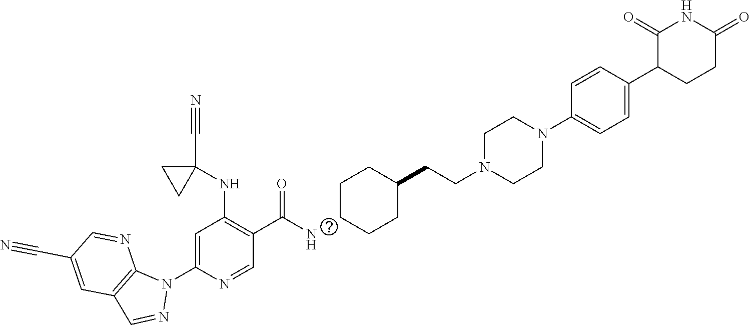 |
| P-123 | 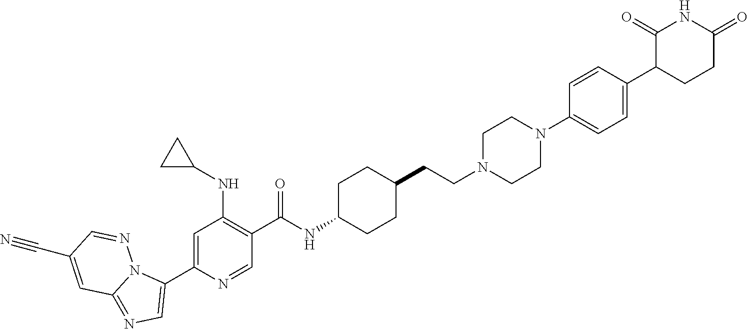 [SMILES: N#Cc1cnn2c(-c3cc(NC4CC4)c(C(=O)N[C@H]4CC[C@H](CCN5CCN(c6ccc(C7CCC(=O)NC7=O)cc6)CC5)CC4)cn3)cnc2c1] |
| P-124 | 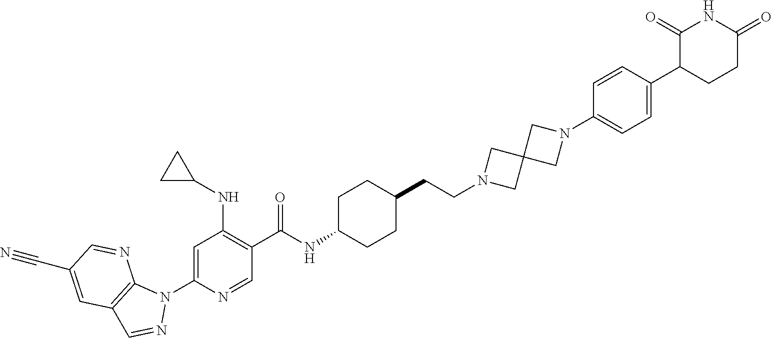 [SMILES: N#Cc1cnc2c(cnn2-c2cc(NC3CC3)c(C(=O)N[C@H]3CC[C@H](CCN4CC5(C4)CN(c4ccc(C6CCC(=O)NC6=O)cc4)C5)CC3)cn2)c1] |
| P-125 | 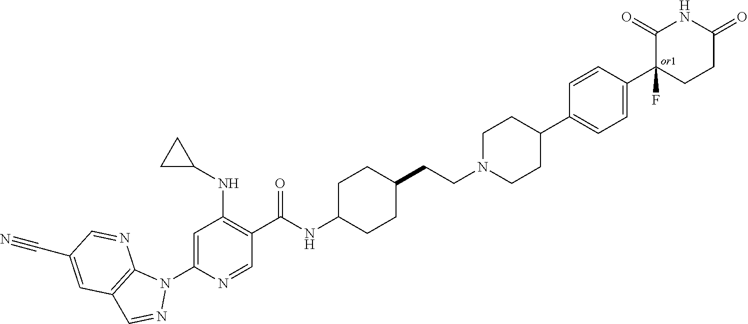 |
| P-126 | 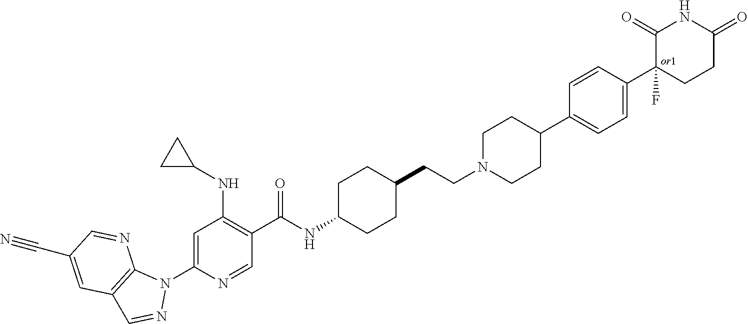 [SMILES: N#Cc1cnc2c(cnn2-c2cc(NC3CC3)c(C(=O)N[C@H]3CC[C@H](CCN4CCC(c5ccc([C@]6(F)CCC(=O)NC6=O)cc5)CC4)CC3)cn2)c1] |
| P-127 | 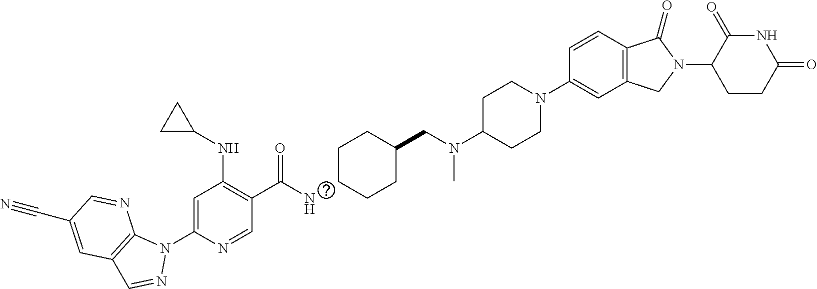 |
| P-128 | 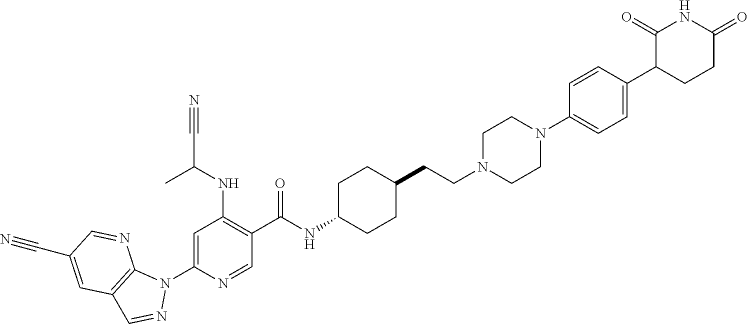 [SMILES: CC(C#N)Nc1cc(-n2ncc3cc(C#N)cnc32)ncc1C(=O)N[C@H]1CC[C@H](CCN2CCN(c3ccc(C4CCC(=O)NC4=O)cc3)CC2)CC1] |
| P-129 | 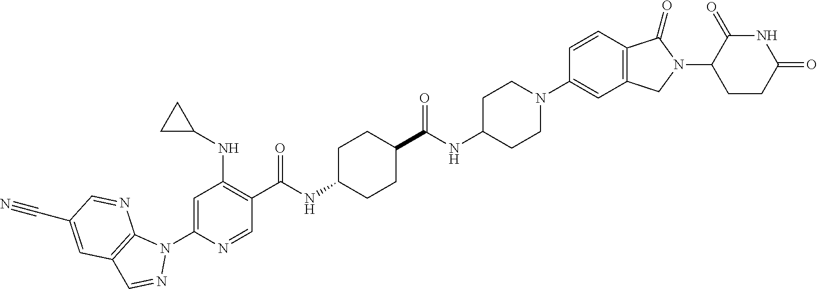 [SMILES: N#Cc1cnc2c(cnn2-c2cc(NC3CC3)c(C(=O)N[C@H]3CC[C@H](C(=O)NC4CCN(c5ccc6c(c5)CN(C5CCC(=O)NC5=O)C6=O)CC4)CC3)cn2)c1] |
| P-130 | 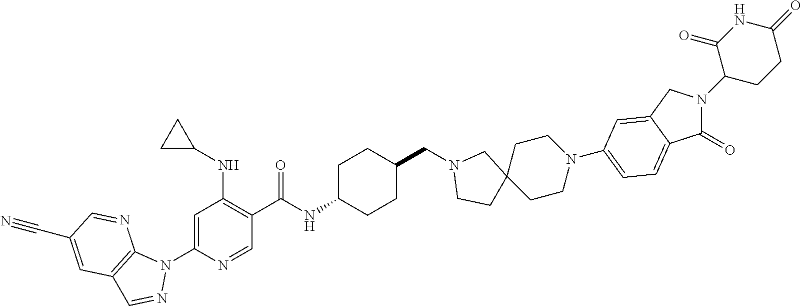 [SMILES: N#Cc1cnc2c(cnn2-c2cc(NC3CC3)c(C(=O)N[C@H]3CC[C@H](CN4CCC5(CCN(c6ccc7c(c6)CN(C6CCC(=O)NC6=O)C7=O)CC5)C4)CC3)cn2)c1] |
| P-131 | 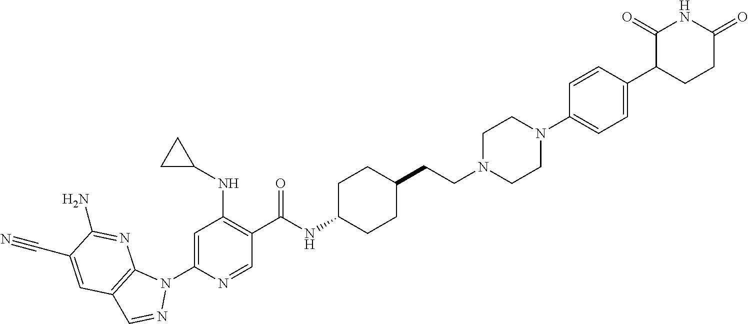 [SMILES: N#Cc1cc2cnn(-c3cc(NC4CC4)c(C(=O)N[C@H]4CC[C@H](CCN5CCN(c6ccc(C7CCC(=O)NC7=O)cc6)CC5)CC4)cn3)c2nc1N] |
| P-132 | 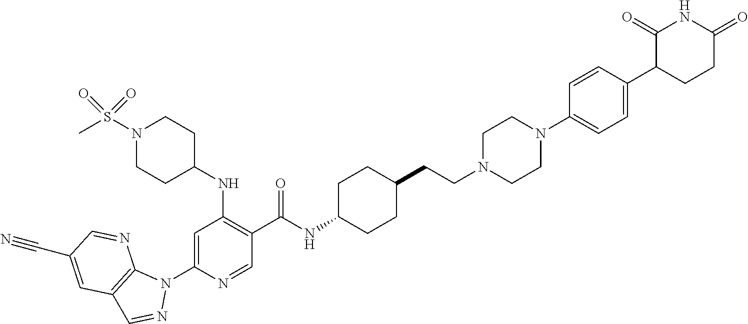 [SMILES: CS(=O)(=O)N1CCC(Nc2cc(-n3ncc4cc(C#N)cnc43)ncc2C(=O)N[C@H]2CC[C@H](CCN3CCN(c4ccc(C5CCC(=O)NC5=O)cc4)CC3)CC2)CC1] |
| P-133 | 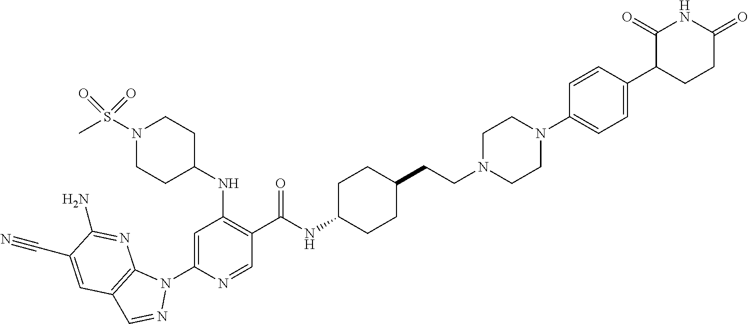 [SMILES: CS(=O)(=O)N1CCC(Nc2cc(-n3ncc4cc(C#N)c(N)nc43)ncc2C(=O)N[C@H]2CC[C@H](CCN3CCN(c4ccc(C5CCC(=O)NC5=O)cc4)CC3)CC2)CC1] |
| P-134 | 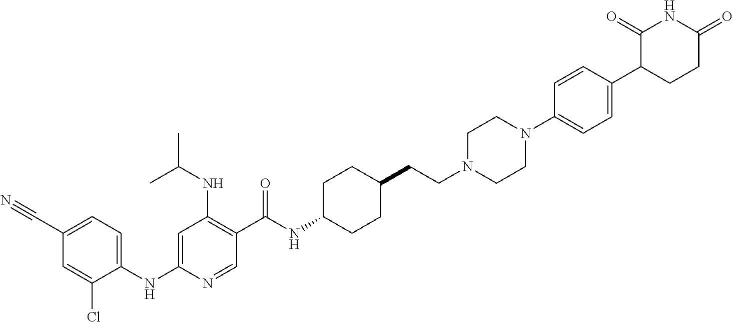 [SMILES: CC(C)Nc1cc(Nc2ccc(C#N)cc2Cl)ncc1C(=O)N[C@H]1CC[C@H](CCN2CCN(c3ccc(C4CCC(=O)NC4=O)cc3)CC2)CC1] |
| P-135 | 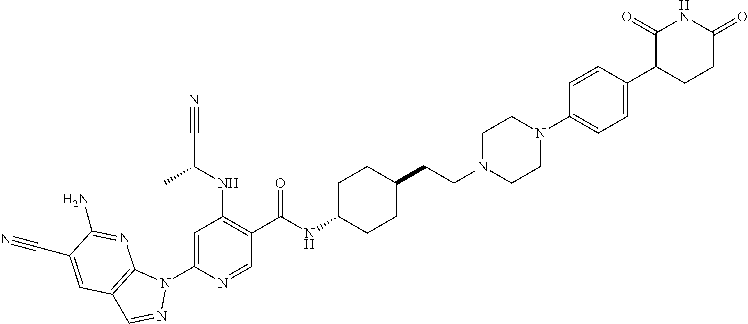 [SMILES: C[C@H](C#N)Nc1cc(-n2ncc3cc(C#N)c(N)nc32)ncc1C(=O)N[C@H]1CC[C@H](CCN2CCN(c3ccc(C4CCC(=O)NC4=O)cc3)CC2)CC1] |
| P-136 | 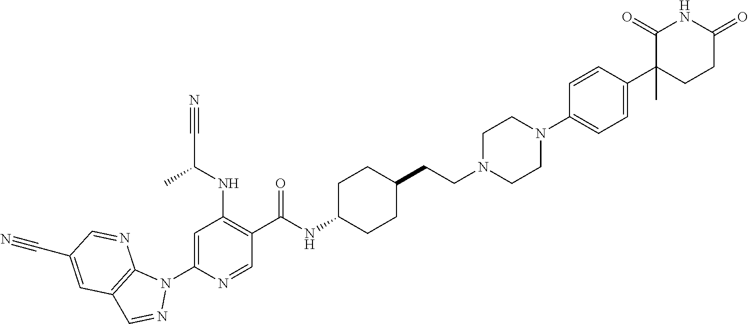 [SMILES: C[C@H](C#N)Nc1cc(-n2ncc3cc(C#N)cnc32)ncc1C(=O)N[C@H]1CC[C@H](CCN2CCN(c3ccc(C4(C)CCC(=O)NC4=O)cc3)CC2)CC1] |
| P-137 | 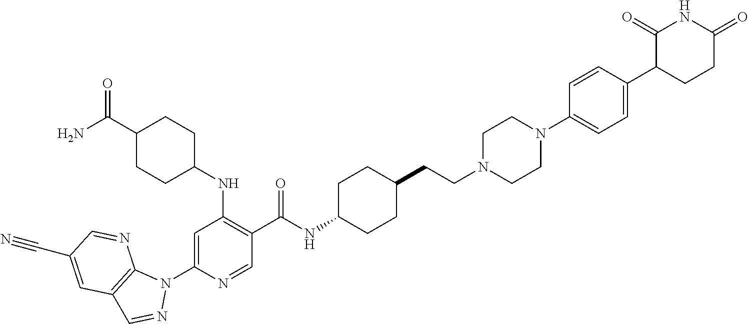 [SMILES: N#Cc1cnc2c(cnn2-c2cc(NC3CCC(C(N)=O)CC3)c(C(=O)N[C@H]3CC[C@H](CCN4CCN(c5ccc(C6CCC(=O)NC6=O)cc5)CC4)CC3)cn2)c1] |
| P-138 | 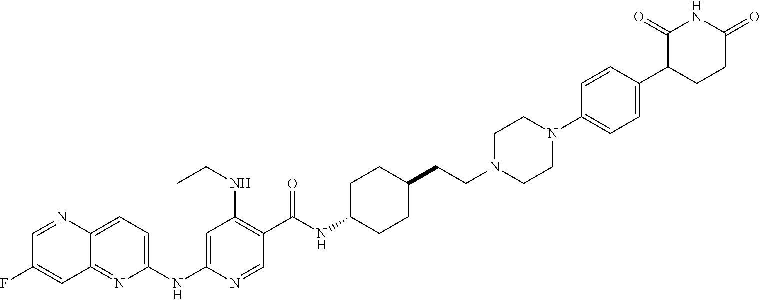 [SMILES: CCNc1cc(Nc2ccc3ncc(F)cc3n2)ncc1C(=O)N[C@H]1CC[C@H](CCN2CCN(c3ccc(C4CCC(=O)NC4=O)cc3)CC2)CC1] |
| P-139 | 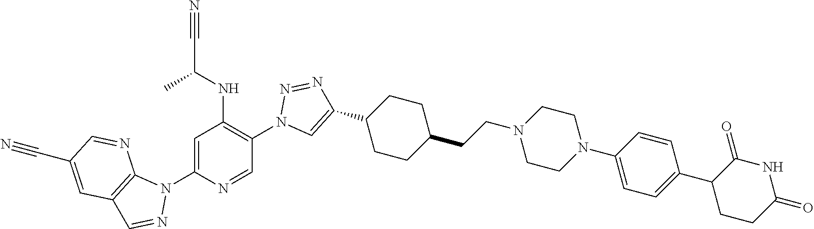 |
| P-140 | 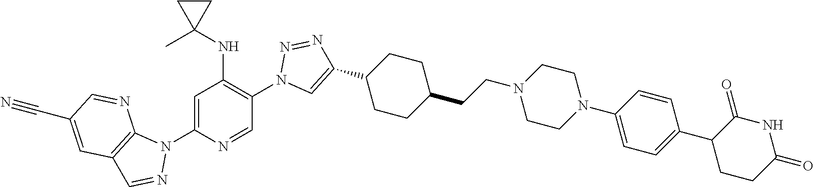 |
| P-141 | 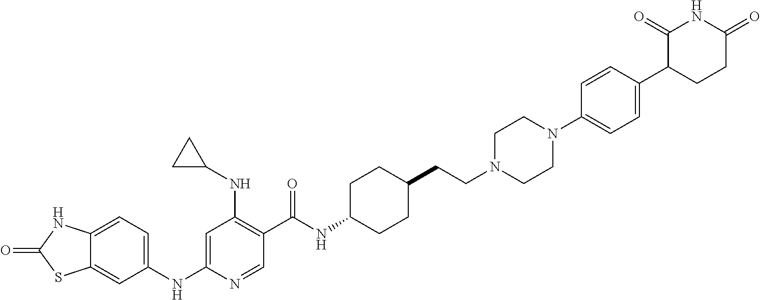 [SMILES: O=C1CCC(c2ccc(N3CCN(CC[C@H]4CC[C@H](NC(=O)c5cnc(Nc6ccc7[nH]c(=O)sc7c6)cc5NC5CC5)CC4)CC3)cc2)C(=O)N1] |
| P-142 | 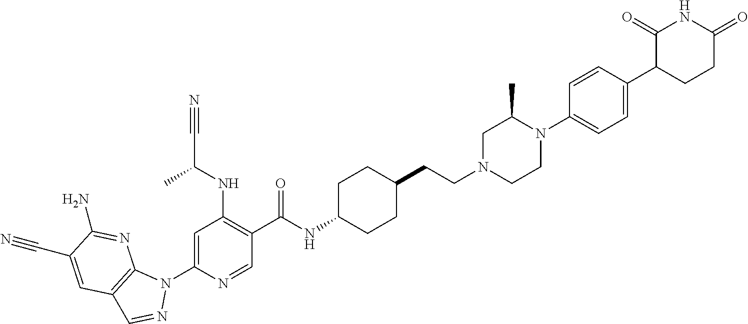 [SMILES: C[C@H](C#N)Nc1cc(-n2ncc3cc(C#N)c(N)nc32)ncc1C(=O)N[C@H]1CC[C@H](CCN2CCN(c3ccc(C4CCC(=O)NC4=O)cc3)[C@H](C)C2)CC1] |
| P-143 | 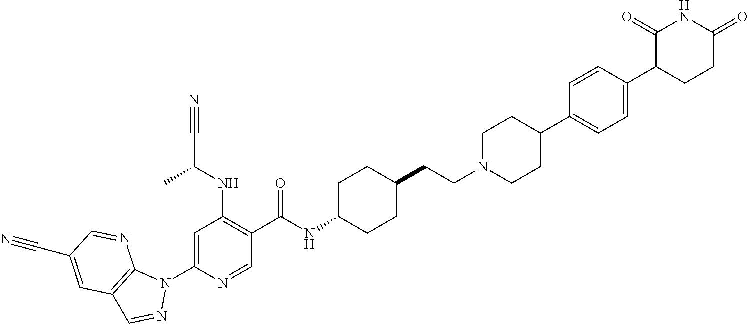 [SMILES: C[C@H](C#N)Nc1cc(-n2ncc3cc(C#N)cnc32)ncc1C(=O)N[C@H]1CC[C@H](CCN2CCC(c3ccc(C4CCC(=O)NC4=O)cc3)CC2)CC1] |
| P-144 | 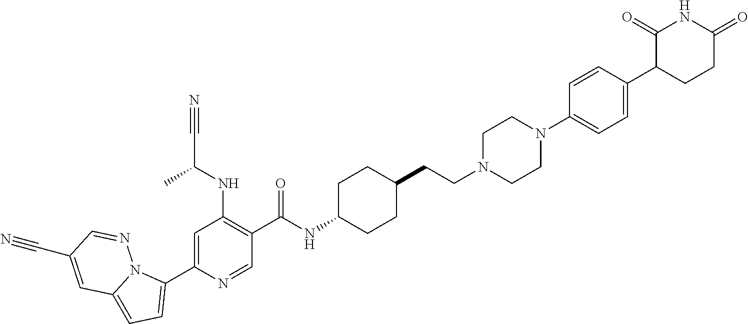 [SMILES: C[C@H](C#N)Nc1cc(-c2ccc3cc(C#N)cnn23)ncc1C(=O)N[C@H]1CC[C@H](CCN2CCN(c3ccc(C4CCC(=O)NC4=O)cc3)CC2)CC1] |
| P-145 | 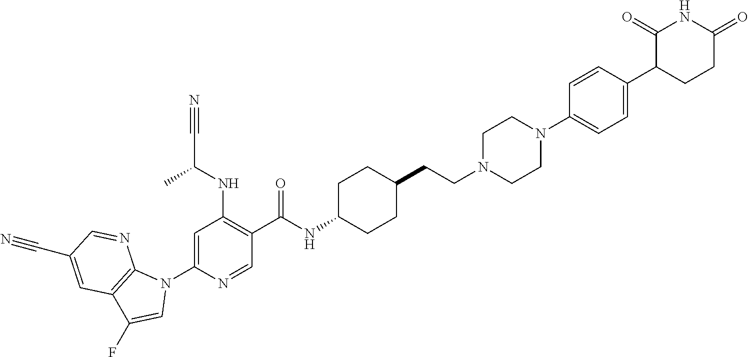 [SMILES: C[C@H](C#N)Nc1cc(-n2cc(F)c3cc(C#N)cnc32)ncc1C(=O)N[C@H]1CC[C@H](CCN2CCN(c3ccc(C4CCC(=O)NC4=O)cc3)CC2)CC1] |
| P-146 | 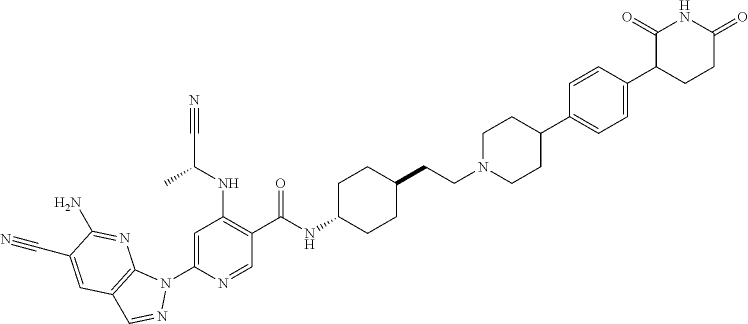 [SMILES: C[C@H](C#N)Nc1cc(-n2ncc3cc(C#N)c(N)nc32)ncc1C(=O)N[C@H]1CC[C@H](CCN2CCC(c3ccc(C4CCC(=O)NC4=O)cc3)CC2)CC1] |
| P-147 | 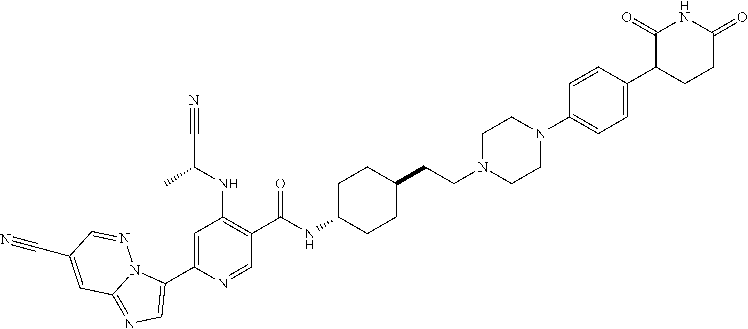 [SMILES: C[C@H](C#N)Nc1cc(-c2cnc3cc(C#N)cnn23)ncc1C(=O)N[C@H]1CC[C@H](CCN2CCN(c3ccc(C4CCC(=O)NC4=O)cc3)CC2)CC1] |
| P-148 | 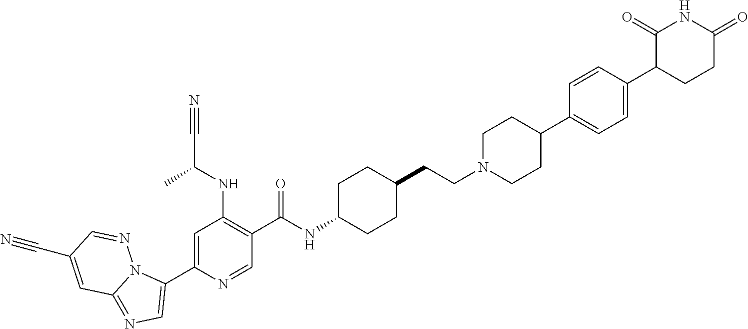 [SMILES: C[C@H](C#N)Nc1cc(-c2cnc3cc(C#N)cnn23)ncc1C(=O)N[C@H]1CC[C@H](CCN2CCC(c3ccc(C4CCC(=O)NC4=O)cc3)CC2)CC1] |
| P-149 | 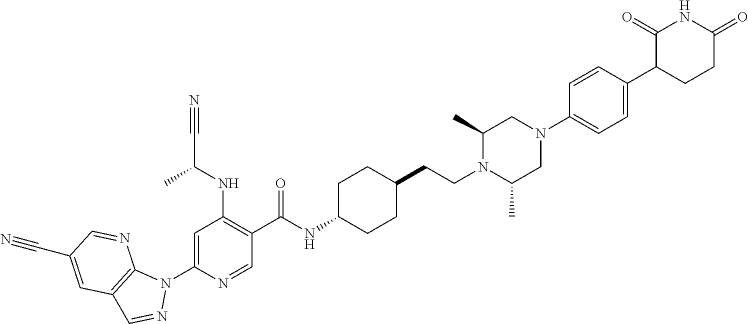 [SMILES: C[C@H](C#N)Nc1cc(-n2ncc3cc(C#N)cnc32)ncc1C(=O)N[C@H]1CC[C@H](CCN2[C@@H](C)CN(c3ccc(C4CCC(=O)NC4=O)cc3)C[C@@H]2C)CC1] |
| P-150 | 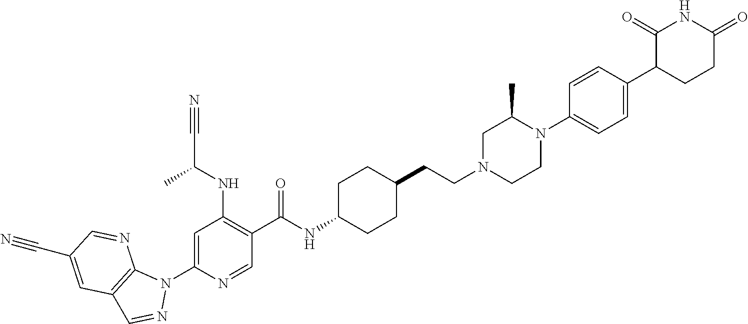 [SMILES: C[C@H](C#N)Nc1cc(-n2ncc3cc(C#N)cnc32)ncc1C(=O)N[C@H]1CC[C@H](CCN2CCN(c3ccc(C4CCC(=O)NC4=O)cc3)[C@H](C)C2)CC1] |
| P-151 | 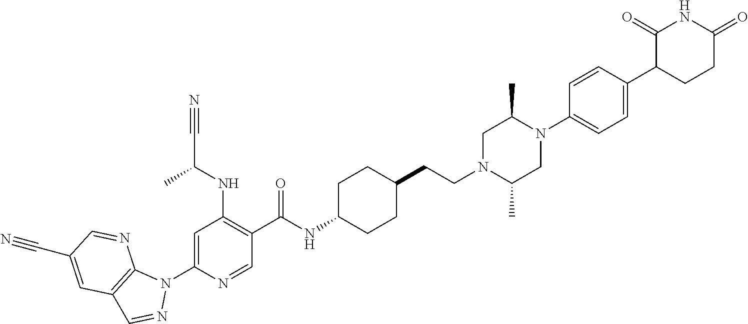 [SMILES: C[C@H](C#N)Nc1cc(-n2ncc3cc(C#N)cnc32)ncc1C(=O)N[C@H]1CC[C@H](CCN2C[C@@H](C)N(c3ccc(C4CCC(=O)NC4=O)cc3)C[C@@H]2C)CC1] |
| P-152 | 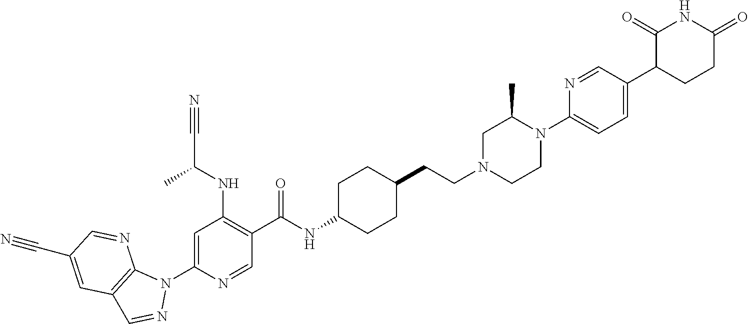 |
| P-153 | 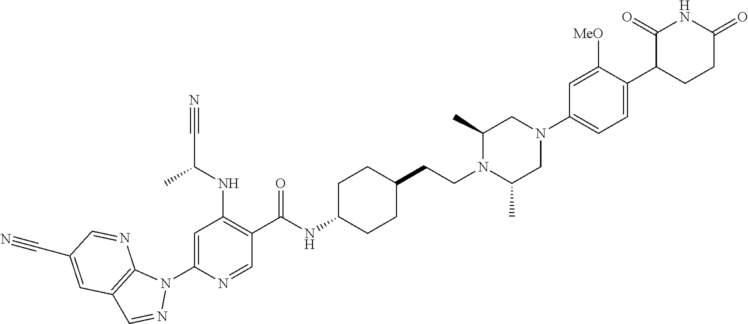 [SMILES: COc1cc(N2C[C@H](C)N(CC[C@H]3CC[C@H](NC(=O)c4cnc(-n5ncc6cc(C#N)cnc65)cc4N[C@H](C)C#N)CC3)[C@@H](C)C2)ccc1C1CCC(=O)NC1=O] |
| P-154 | 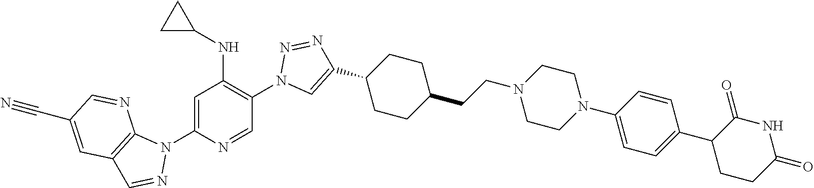 [SMILES: N#Cc1cnc2c(cnn2-c2cc(NC3CC3)c(-n3cc([C@H]4CC[C@H](CCN5CCN(c6ccc(C7CCC(=O)NC7=O)cc6)CC5)CC4)nn3)cn2)c1] |
| P-155 | 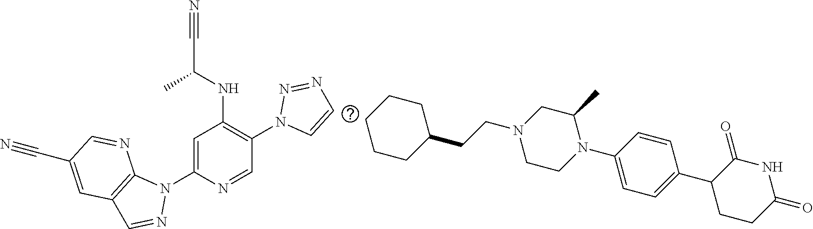 |
| P-156 | 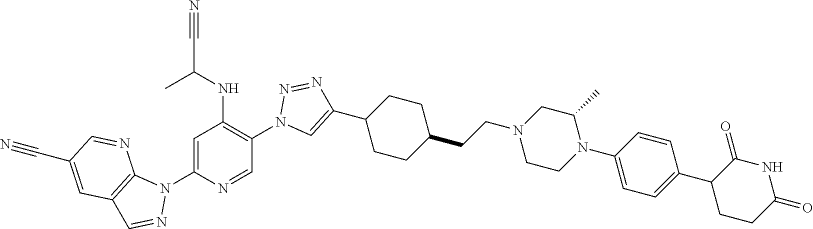 |
| P-157 | 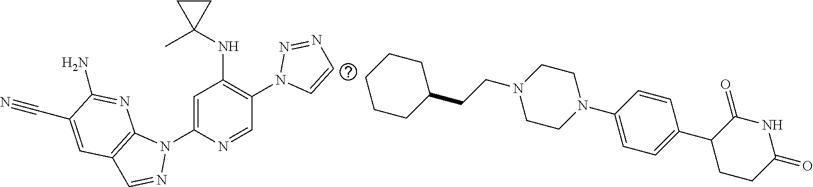 |
| P-158 | 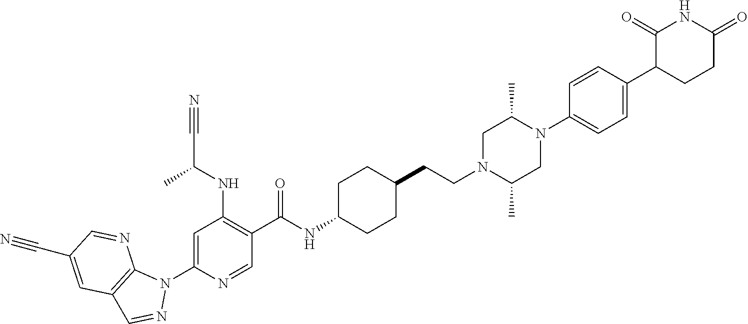 [SMILES: C[C@H](C#N)Nc1cc(-n2ncc3cc(C#N)cnc32)ncc1C(=O)N[C@H]1CC[C@H](CCN2C[C@H](C)N(c3ccc(C4CCC(=O)NC4=O)cc3)C[C@@H]2C)CC1] |
| P-159 | 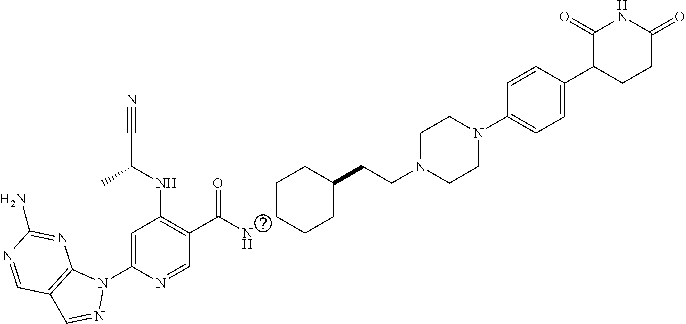 |
| P-160 | 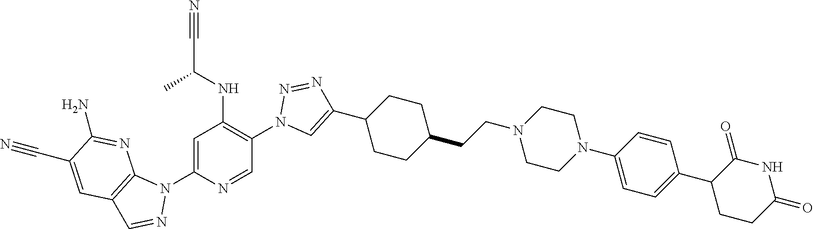 |
| P-161 | 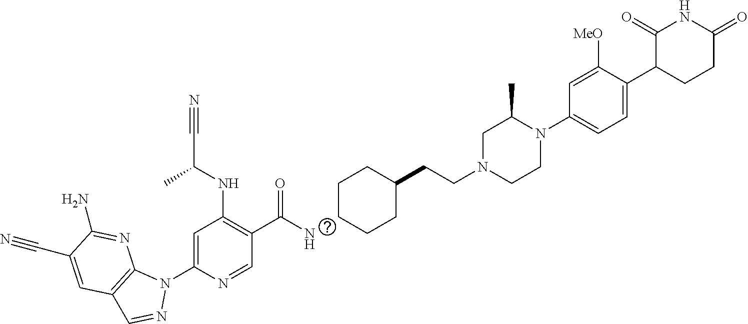 |
| P-162 | 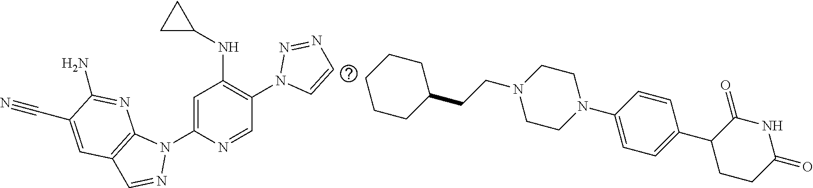 |
| P-163 | 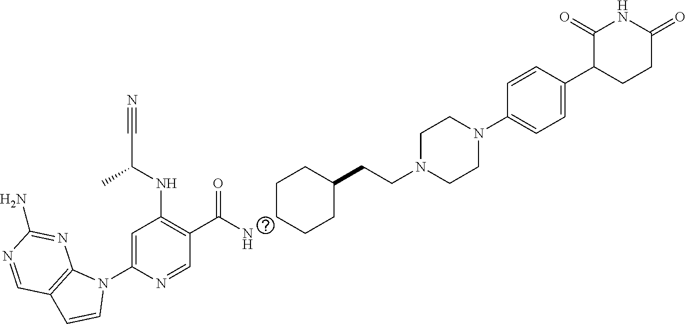 |
| P-164 | 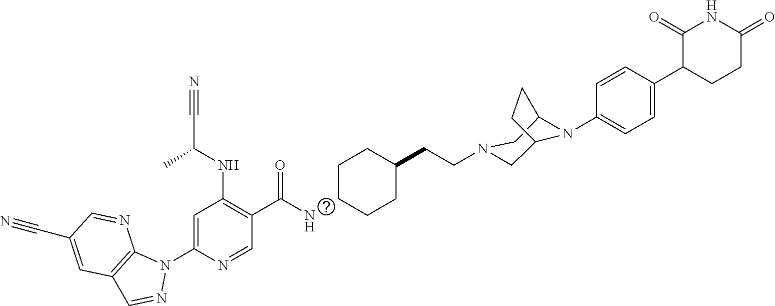 |
| P-165 | 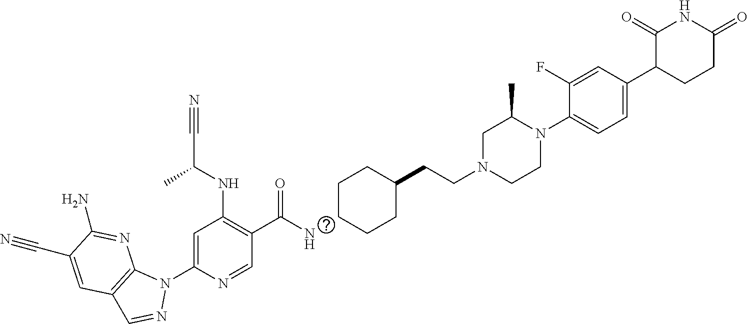 |
| P-166 | 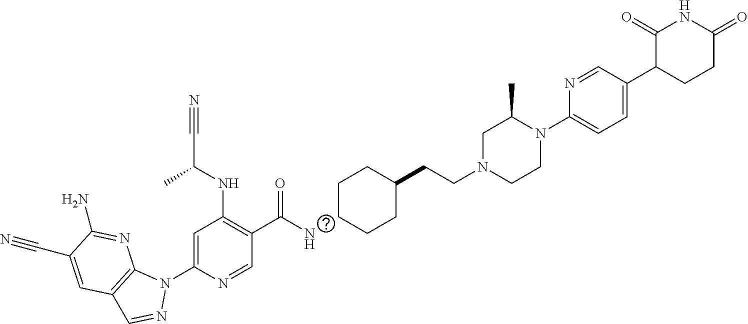 |
| P-167 | 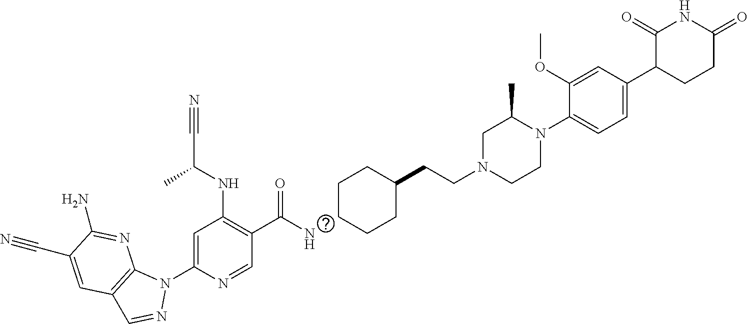 |
| P-168 | 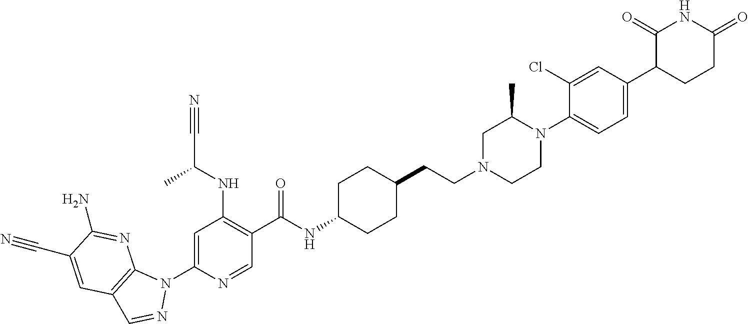 [SMILES: C[C@H](C#N)Nc1cc(-n2ncc3cc(C#N)c(N)nc32)ncc1C(=O)N[C@H]1CC[C@H](CCN2CCN(c3ccc(C4CCC(=O)NC4=O)cc3Cl)[C@H](C)C2)CC1] |
| P-169 | 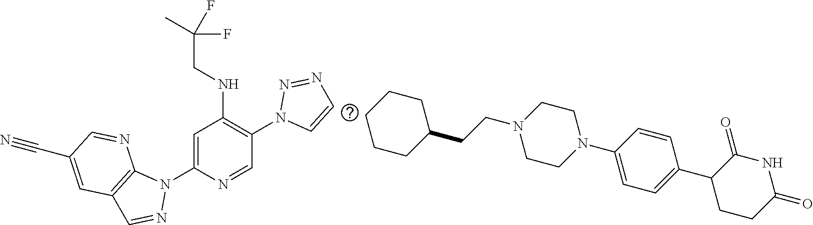 |
| P-170 | 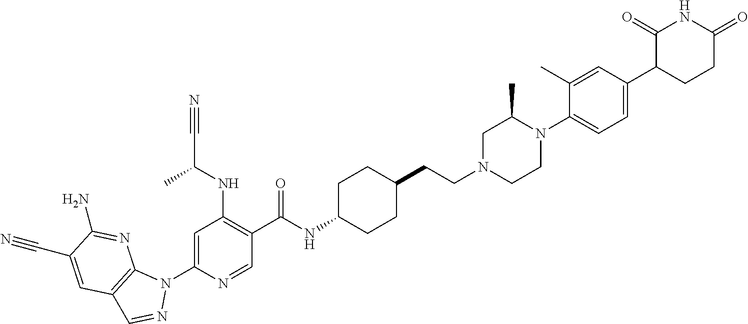 [SMILES: Cc1cc(C2CCC(=O)NC2=O)ccc1N1CCN(CC[C@H]2CC[C@H](NC(=O)c3cnc(-n4ncc5cc(C#N)c(N)nc54)cc3N[C@H](C)C#N)CC2)C[C@H]1C] |
| P-171 | 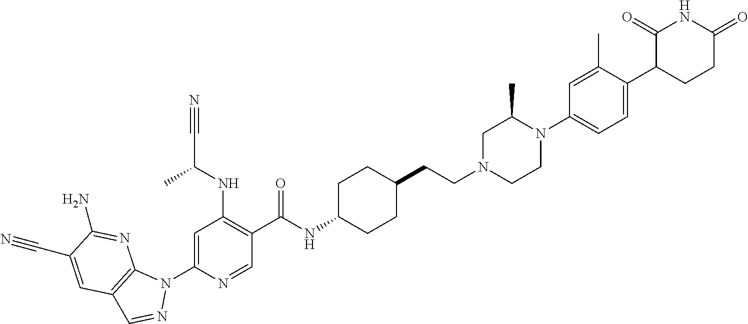 [SMILES: Cc1cc(N2CCN(CC[C@H]3CC[C@H](NC(=O)c4cnc(-n5ncc6cc(C#N)c(N)nc65)cc4N[C@H](C)C#N)CC3)C[C@H]2C)ccc1C1CCC(=O)NC1=O] |
| P-172 | 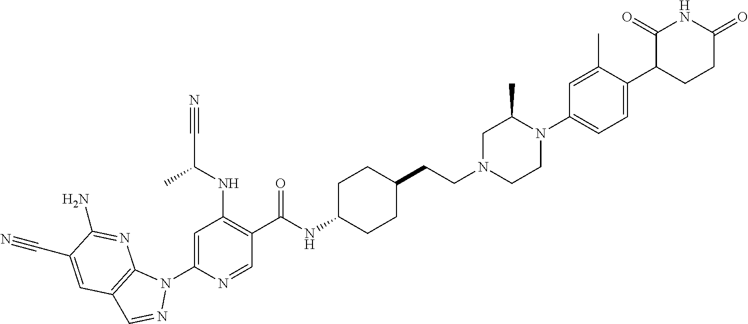 [SMILES: Cc1cc(N2CCN(CC[C@H]3CC[C@H](NC(=O)c4cnc(-n5ncc6cc(C#N)c(N)nc65)cc4N[C@H](C)C#N)CC3)C[C@H]2C)ccc1C1CCC(=O)NC1=O] |
| P-173 | 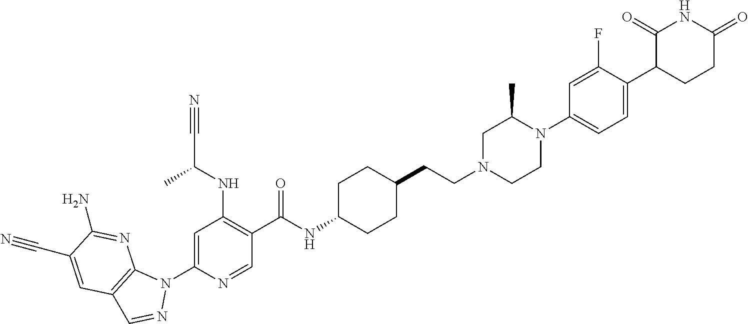 [SMILES: C[C@H](C#N)Nc1cc(-n2ncc3cc(C#N)c(N)nc32)ncc1C(=O)N[C@H]1CC[C@H](CCN2CCN(c3ccc(C4CCC(=O)NC4=O)c(F)c3)[C@H](C)C2)CC1] |
| P-174 | 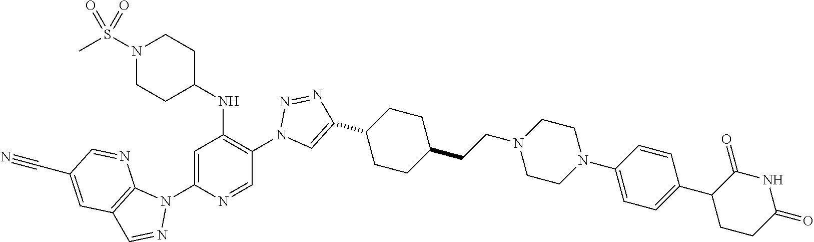 |
| P-175 | 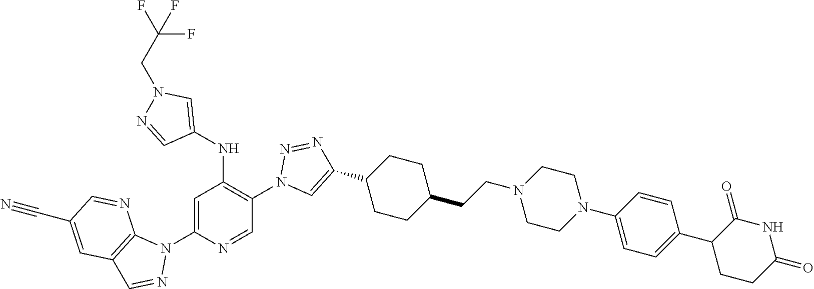 |
| P-176 | 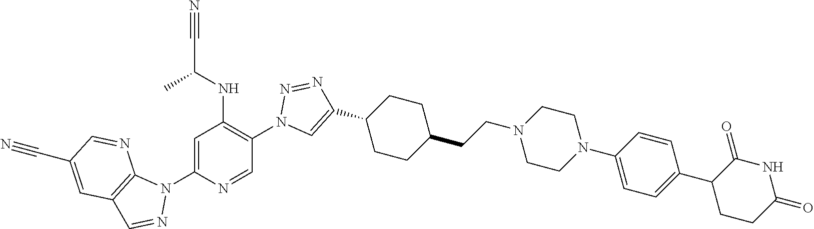 [SMILES: C[C@H](C#N)Nc1cc(-n2ncc3cc(C#N)cnc32)ncc1-n1cc([C@H]2CC[C@H](CCN3CCN(c4ccc(C5CCC(=O)NC5=O)cc4)CC3)CC2)nn1] |
| P-177 | 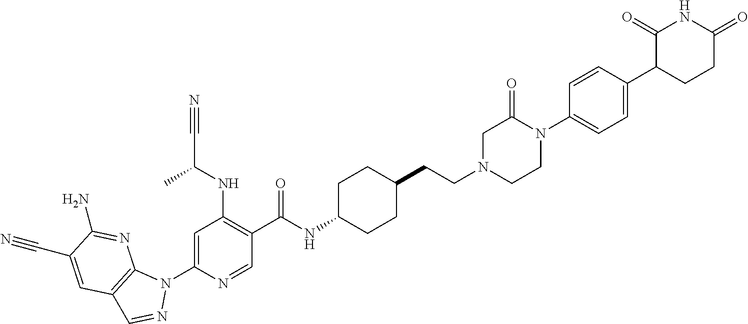 [SMILES: C[C@H](C#N)Nc1cc(-n2ncc3cc(C#N)c(N)nc32)ncc1C(=O)N[C@H]1CC[C@H](CCN2CCN(c3ccc(C4CCC(=O)NC4=O)cc3)C(=O)C2)CC1] |
| P-178 | 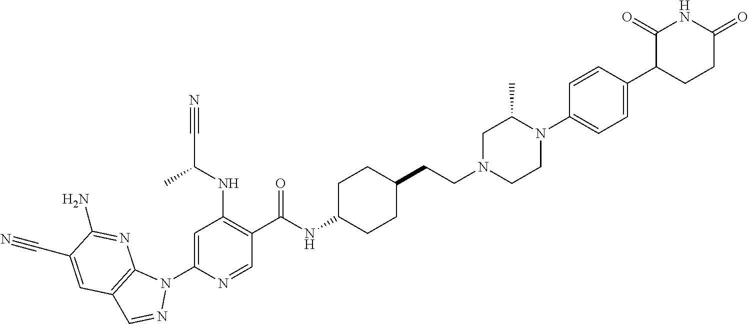 |
| P-179 | 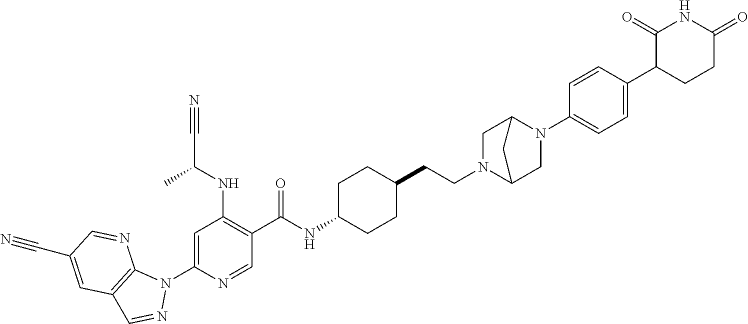 [SMILES: C[C@H](C#N)Nc1cc(-n2ncc3cc(C#N)cnc32)ncc1C(=O)N[C@H]1CC[C@H](CCN2CC3CC2CN3c2ccc(C3CCC(=O)NC3=O)cc2)CC1] |
| P-180 | 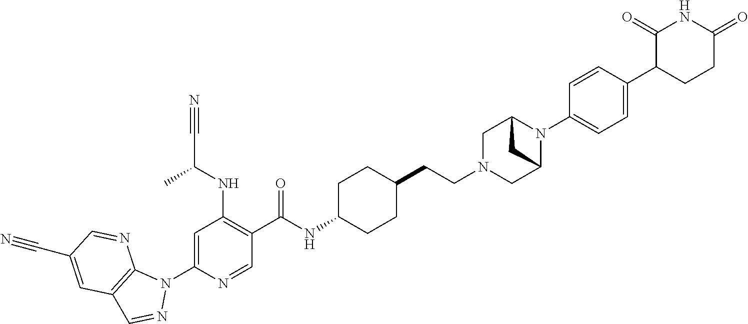 [SMILES: C[C@H](C#N)Nc1cc(-n2ncc3cc(C#N)cnc32)ncc1C(=O)N[C@H]1CC[C@H](CCN2C[C@H]3C[C@@H](C2)N3c2ccc(C3CCC(=O)NC3=O)cc2)CC1] |
| P-181 | 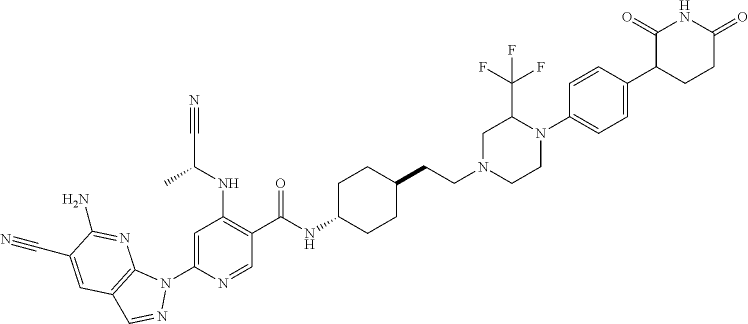 [SMILES: C[C@H](C#N)Nc1cc(-n2ncc3cc(C#N)c(N)nc32)ncc1C(=O)N[C@H]1CC[C@H](CCN2CCN(c3ccc(C4CCC(=O)NC4=O)cc3)C(C(F)(F)F)C2)CC1] |
| P-182 | 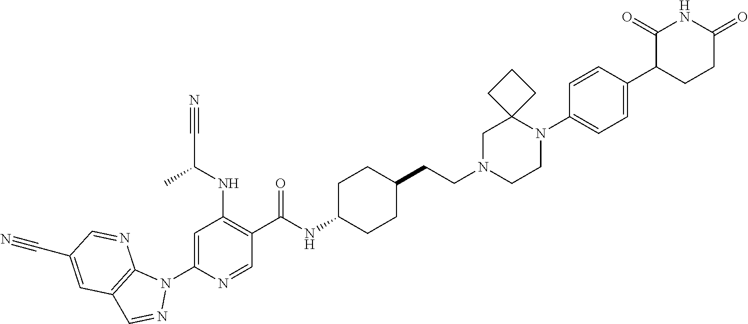 [SMILES: C[C@H](C#N)Nc1cc(-n2ncc3cc(C#N)cnc32)ncc1C(=O)N[C@H]1CC[C@H](CCN2CCN(c3ccc(C4CCC(=O)NC4=O)cc3)C3(CCC3)C2)CC1] |
| P-183 | 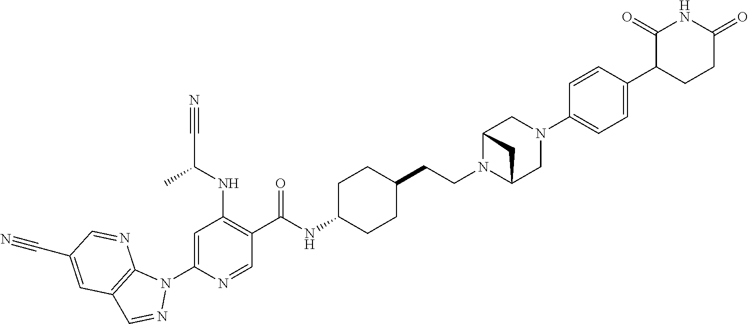 [SMILES: C[C@H](C#N)Nc1cc(-n2ncc3cc(C#N)cnc32)ncc1C(=O)N[C@H]1CC[C@H](CCN2[C@@H]3C[C@H]2CN(c2ccc(C4CCC(=O)NC4=O)cc2)C3)CC1] |
| P-184 | 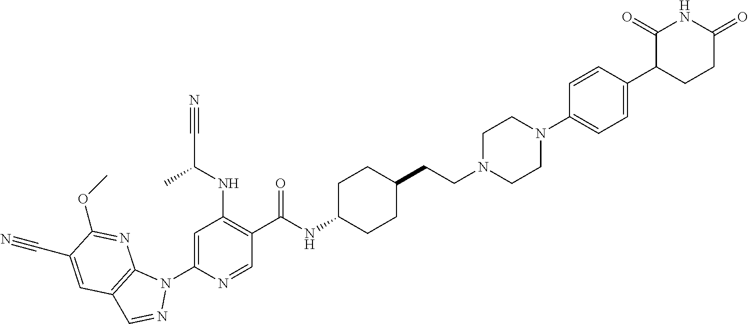 [SMILES: COc1nc2c(cnn2-c2cc(N[C@H](C)C#N)c(C(=O)N[C@H]3CC[C@H](CCN4CCN(c5ccc(C6CCC(=O)NC6=O)cc5)CC4)CC3)cn2)cc1C#N] |
| P-185 | 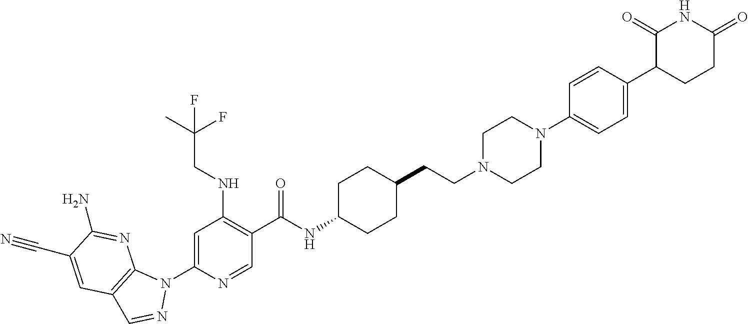 [SMILES: CC(F)(F)CNc1cc(-n2ncc3cc(C#N)c(N)nc32)ncc1C(=O)N[C@H]1CC[C@H](CCN2CCN(c3ccc(C4CCC(=O)NC4=O)cc3)CC2)CC1] |
| P-186 | 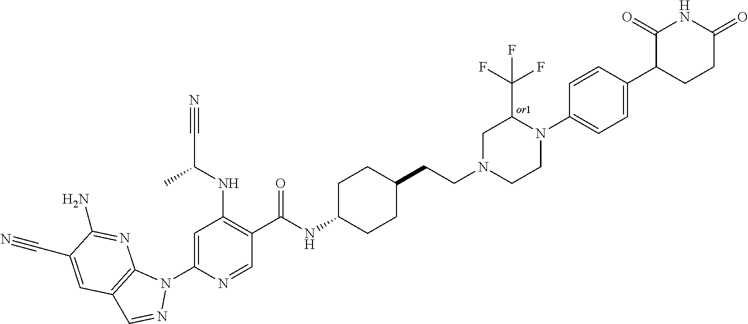 [SMILES: C[C@H](C#N)Nc1cc(-n2ncc3cc(C#N)c(N)nc32)ncc1C(=O)N[C@H]1CC[C@H](CCN2CCN(c3ccc(C4CCC(=O)NC4=O)cc3)C(C(F)(F)F)C2)CC1] |
| P-187 | 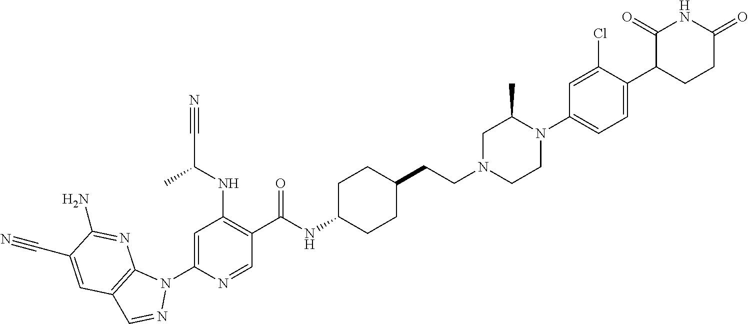 [SMILES: C[C@H](C#N)Nc1cc(-n2ncc3cc(C#N)c(N)nc32)ncc1C(=O)N[C@H]1CC[C@H](CCN2CCN(c3ccc(C4CCC(=O)NC4=O)c(Cl)c3)[C@H](C)C2)CC1] |
| P-188 | 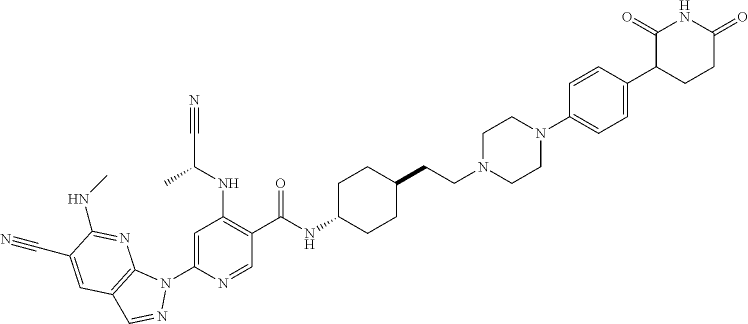 [SMILES: CNc1nc2c(cnn2-c2cc(N[C@H](C)C#N)c(C(=O)N[C@H]3CC[C@H](CCN4CCN(c5ccc(C6CCC(=O)NC6=O)cc5)CC4)CC3)cn2)cc1C#N] |
| P-189 | 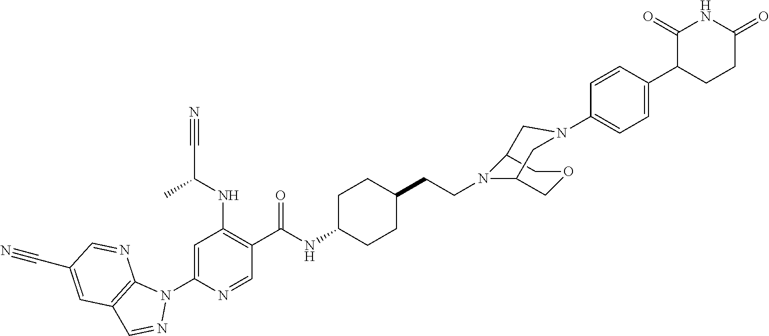 [SMILES: C[C@H](C#N)Nc1cc(-n2ncc3cc(C#N)cnc32)ncc1C(=O)N[C@H]1CC[C@H](CCN2C3COCC2CN(c2ccc(C4CCC(=O)NC4=O)cc2)C3)CC1] |
| P-190 | 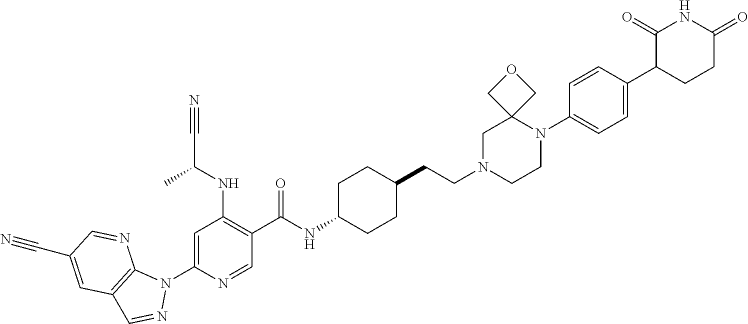 [SMILES: C[C@H](C#N)Nc1cc(-n2ncc3cc(C#N)cnc32)ncc1C(=O)N[C@H]1CC[C@H](CCN2CCN(c3ccc(C4CCC(=O)NC4=O)cc3)C3(COC3)C2)CC1] |
| P-191 | 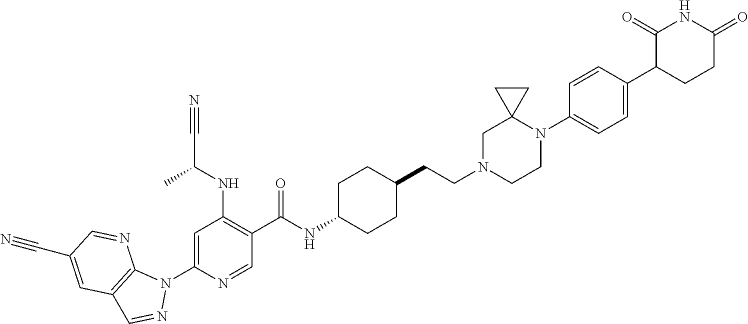 [SMILES: C[C@H](C#N)Nc1cc(-n2ncc3cc(C#N)cnc32)ncc1C(=O)N[C@H]1CC[C@H](CCN2CCN(c3ccc(C4CCC(=O)NC4=O)cc3)C3(CC3)C2)CC1] |
| P-192 | 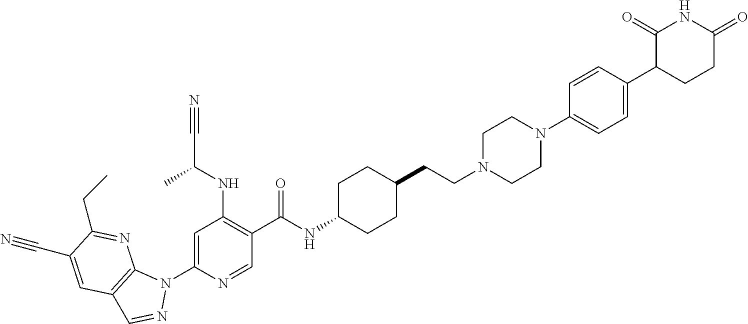 [SMILES: CCc1nc2c(cnn2-c2cc(N[C@H](C)C#N)c(C(=O)N[C@H]3CC[C@H](CCN4CCN(c5ccc(C6CCC(=O)NC6=O)cc5)CC4)CC3)cn2)cc1C#N] |
| P-193 | 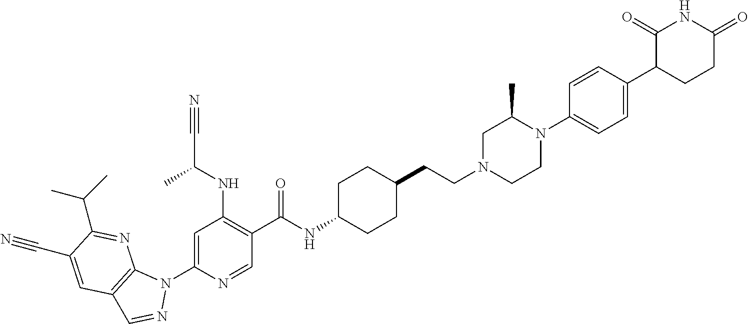 [SMILES: CC(C)c1nc2c(cnn2-c2cc(N[C@H](C)C#N)c(C(=O)N[C@H]3CC[C@H](CCN4CCN(c5ccc(C6CCC(=O)NC6=O)cc5)[C@H](C)C4)CC3)cn2)cc1C#N] |
| P-194 | 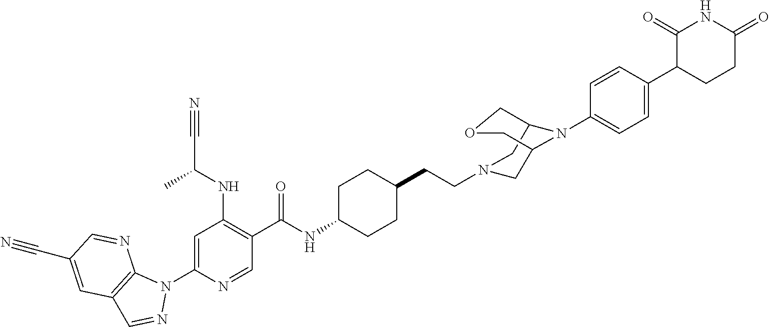 [SMILES: C[C@H](C#N)Nc1cc(-n2ncc3cc(C#N)cnc32)ncc1C(=O)N[C@H]1CC[C@H](CCN2CC3COCC(C2)N3c2ccc(C3CCC(=O)NC3=O)cc2)CC1] |
| P-195 | 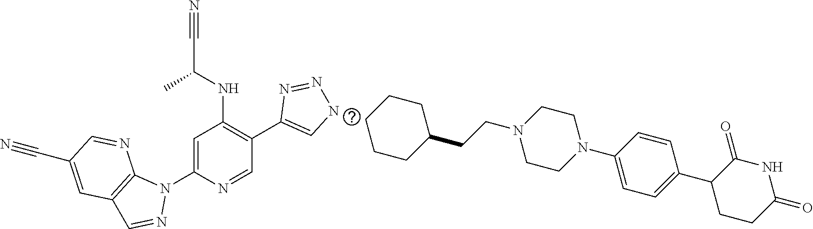 |
| P-196 | 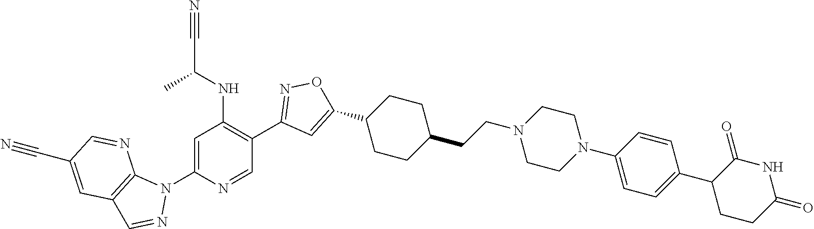 |
| P-197 | 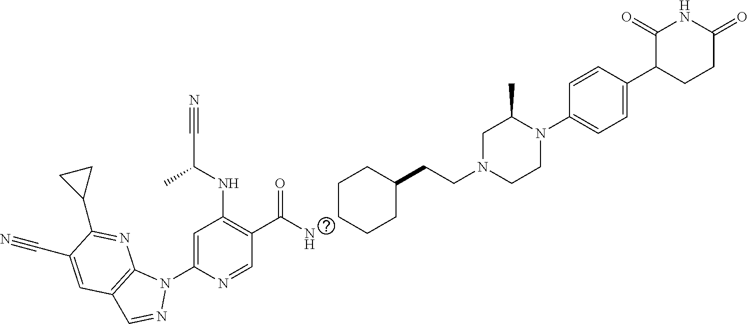 |
| P-198 | 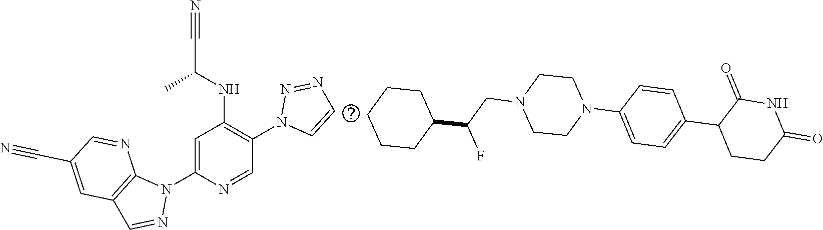 |
| P-199 | 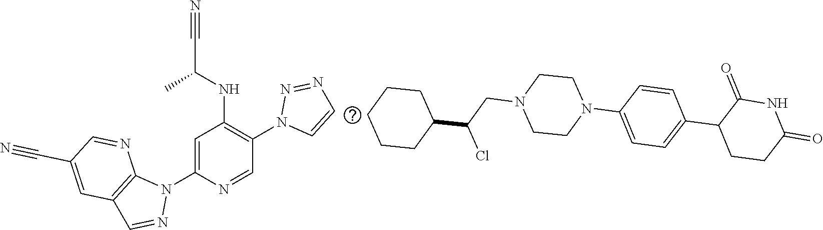 |
| P-200 | 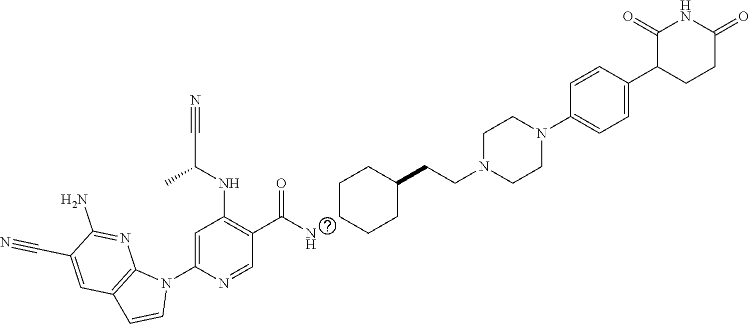 |
| P-201 | 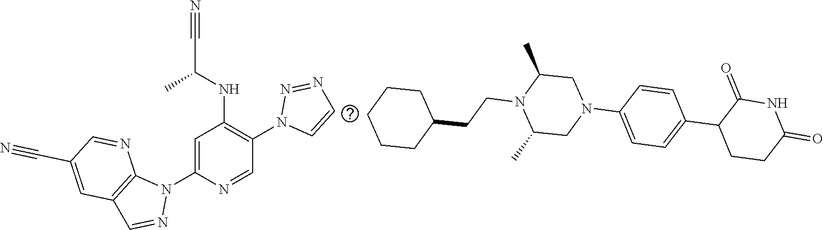 |
| P-202 | 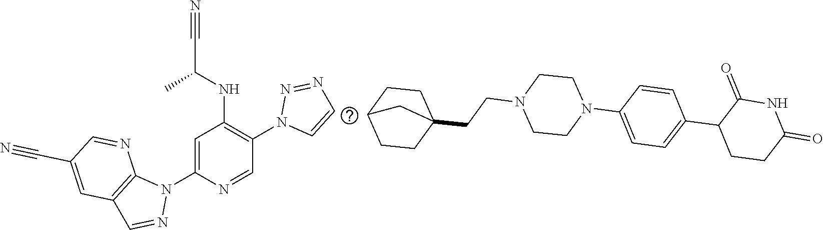 |
| P-203 | 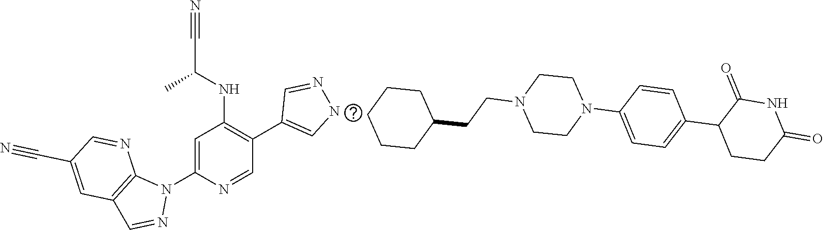 |
| P-204 | 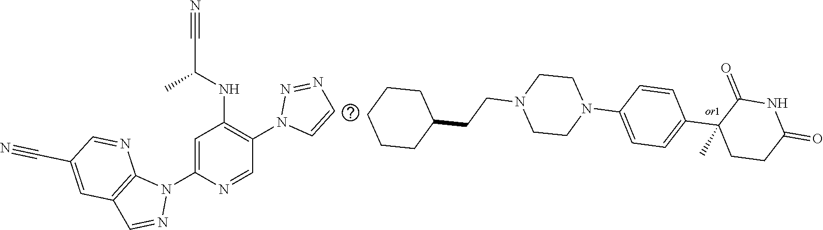 |
| P-205 | 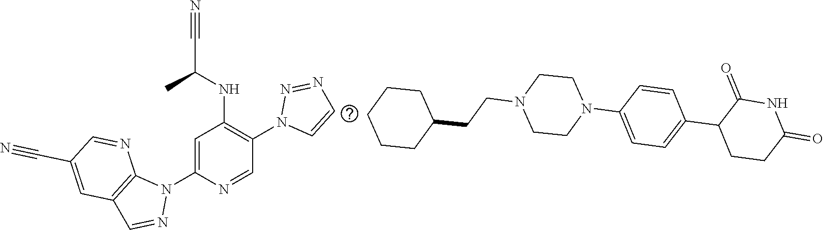 |
| P-206 | 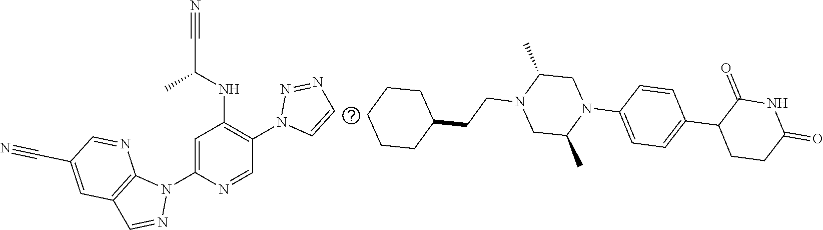 |
| P-207 | 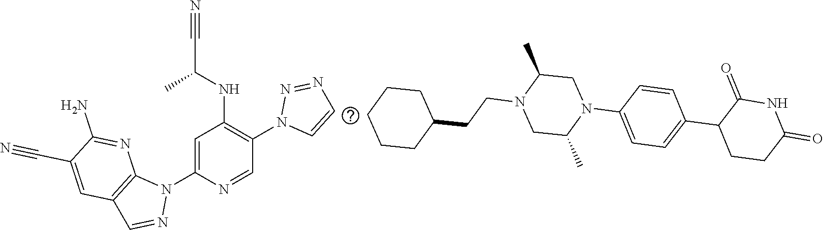 |
| P-208 | 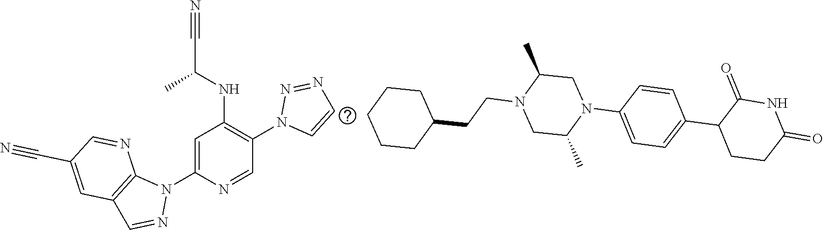 |
| P-209 | 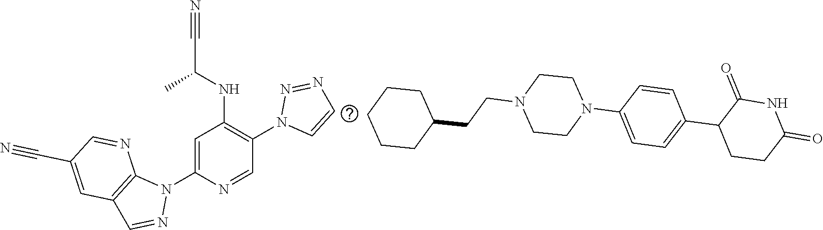 |
| P-210 | 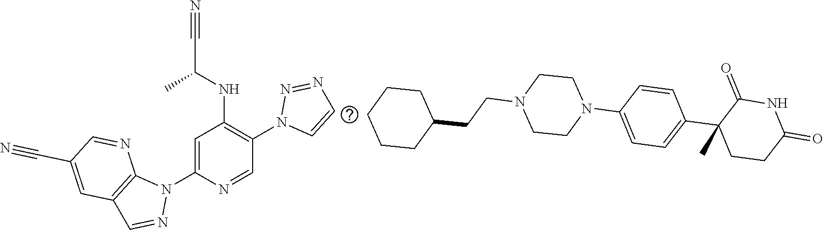 |
| P-211 | 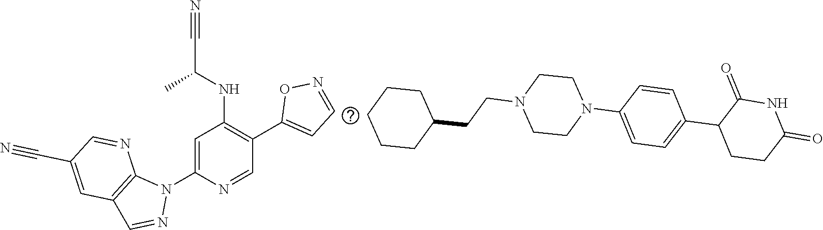 |
| P-212 | 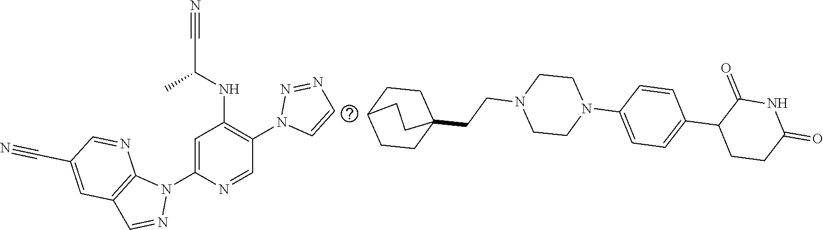 |
| P-213 | 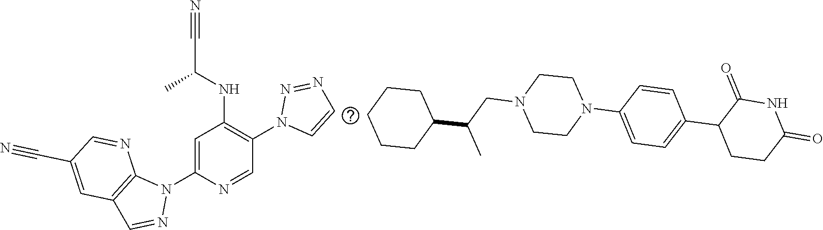 |
| P-214 | 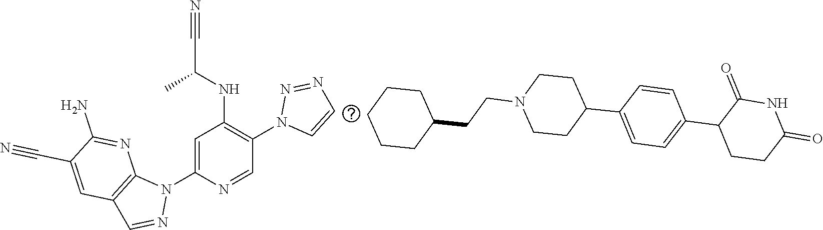 |
| P-215 | 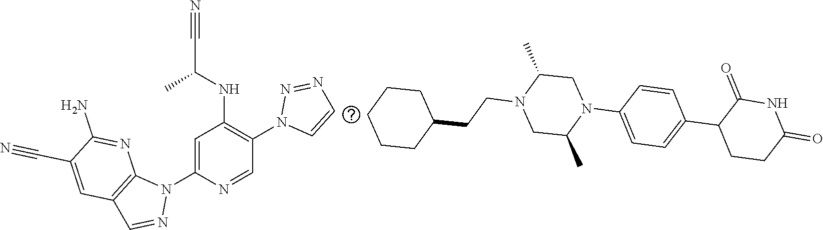 |
| P-216 | 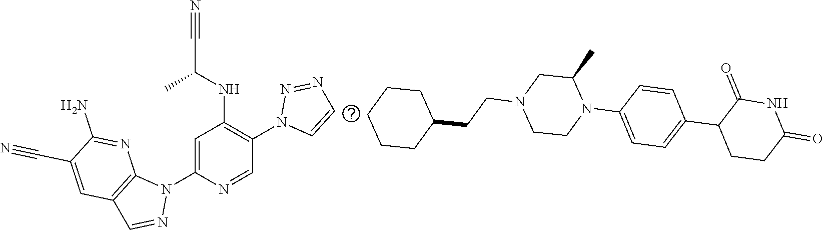 |
| P-217 | 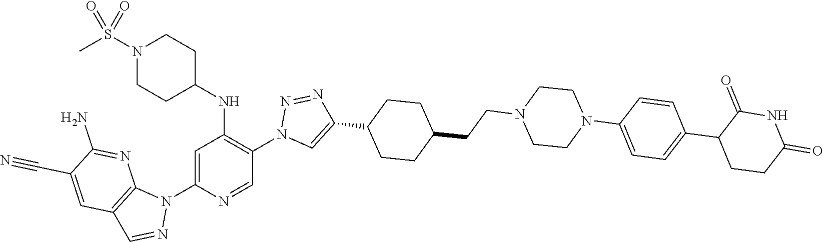 |
| P-218 | 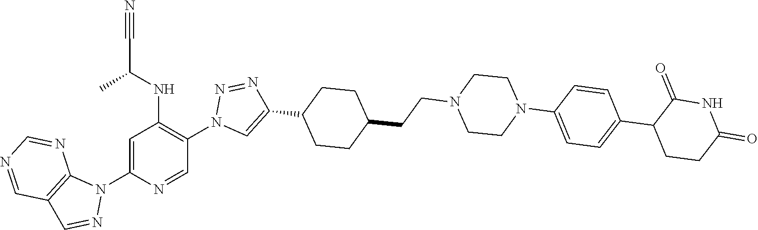 |
| P-219 | 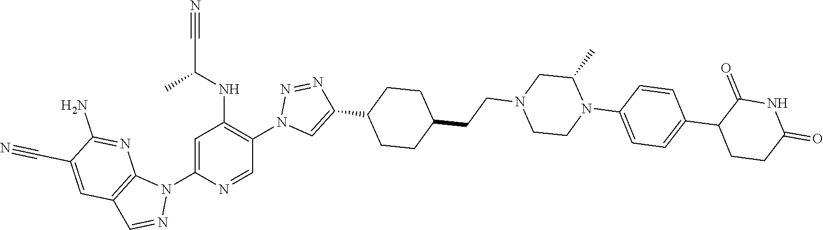 |
| P-220 | 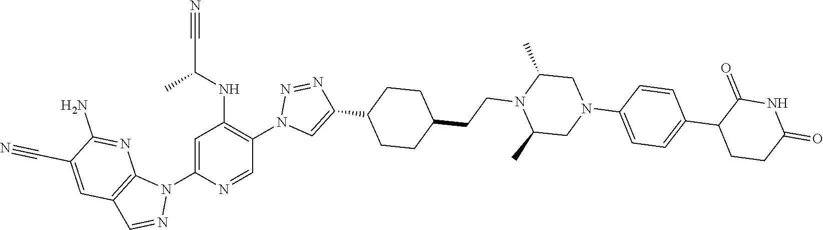 |
| P-221 | 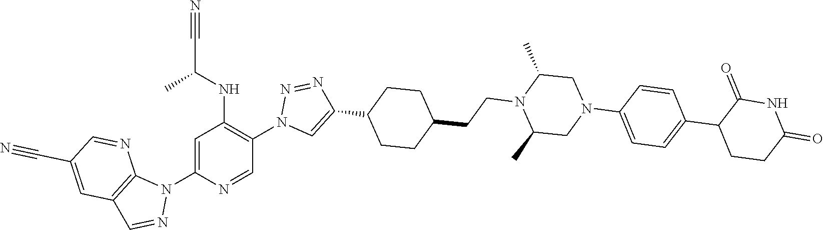 |
| P-222 | 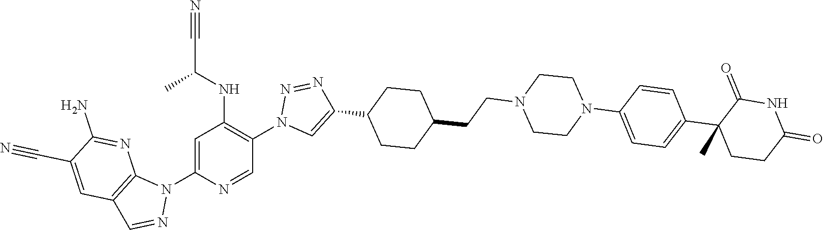 |
| P-223 | 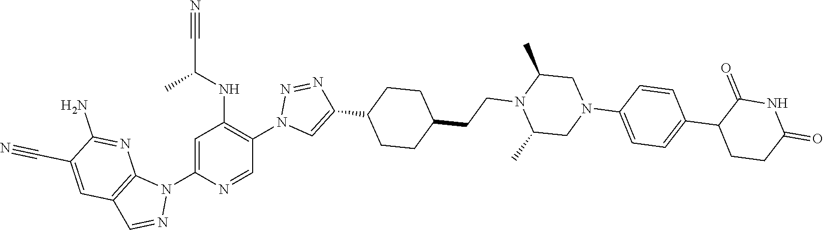 |
| P-224 | 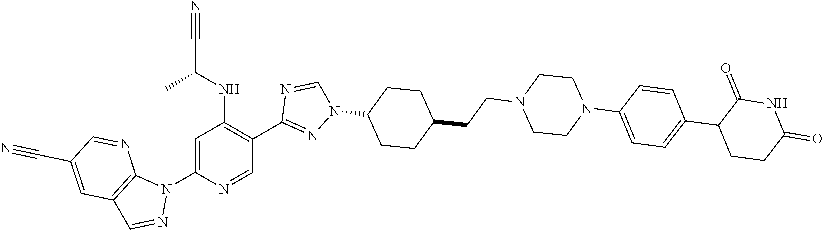 |
| P-225 | 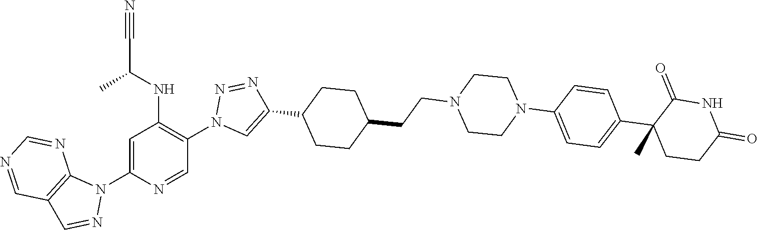 |
| P-226 | 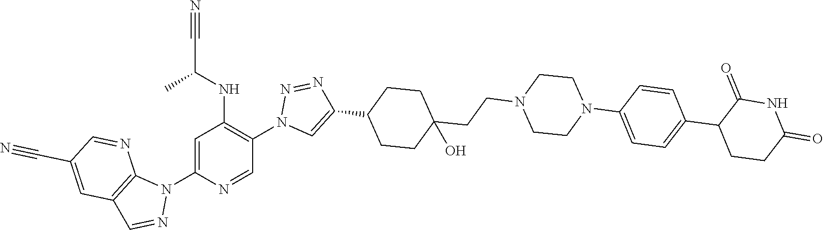 |
| P-227 | 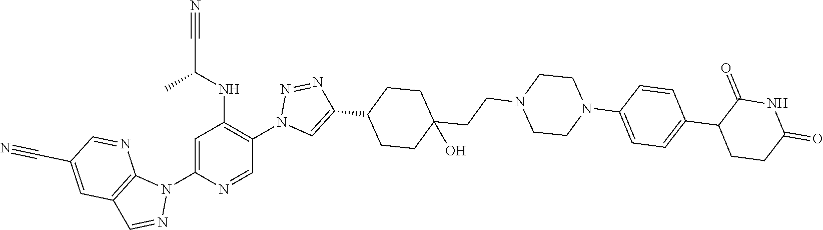 |
| P-228 | 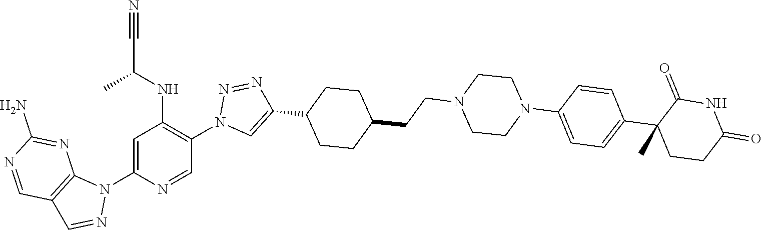 |
| P-229 | 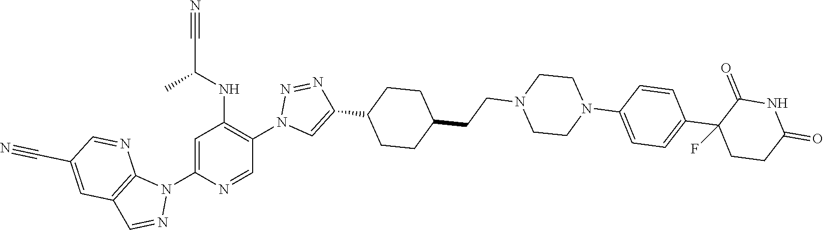 |
| P-230 | 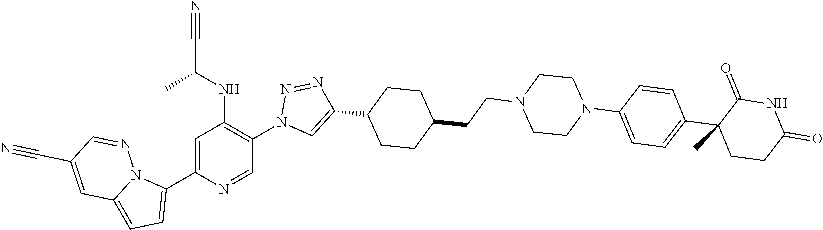 |
| P-231 | 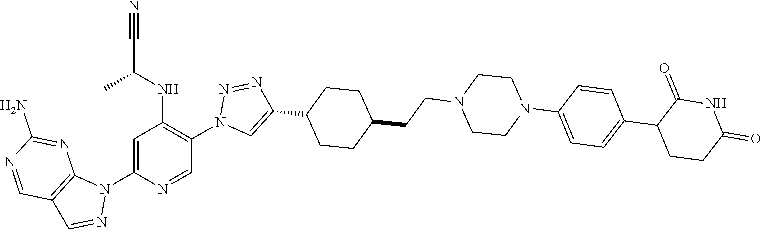 |
| P-232 | 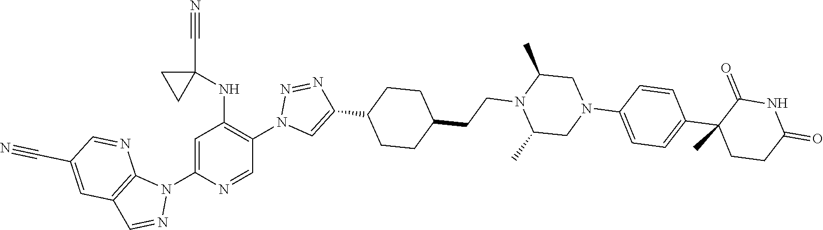 |
| P-233 | 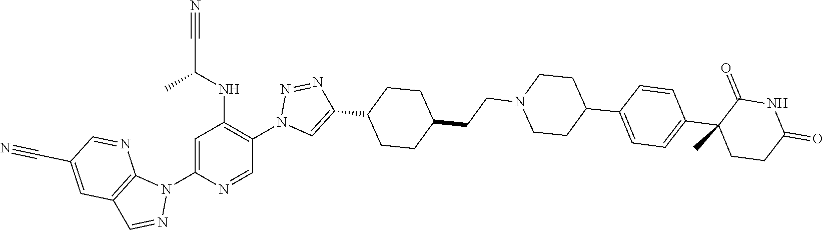 |
| P-234 | 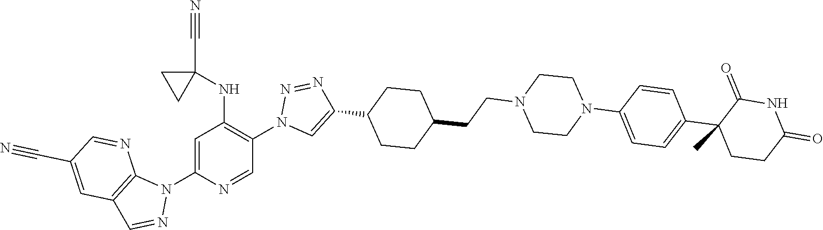 |
| P-235 | 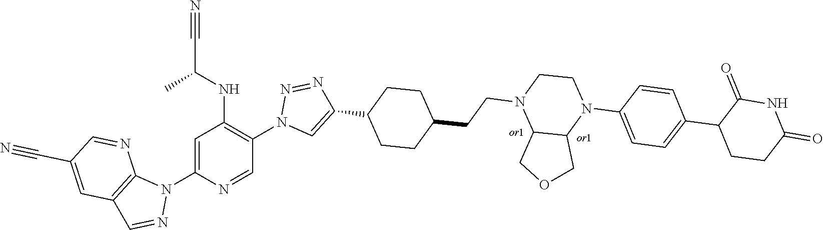 |
| P-236 | 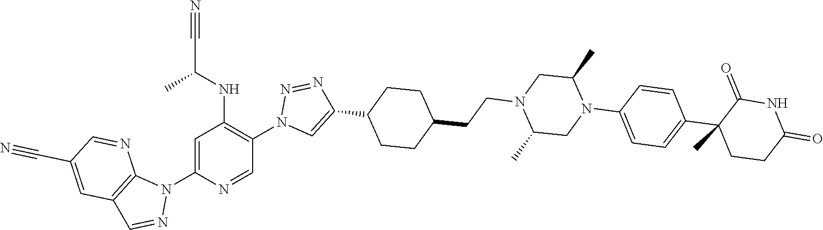 |
| P-237 | 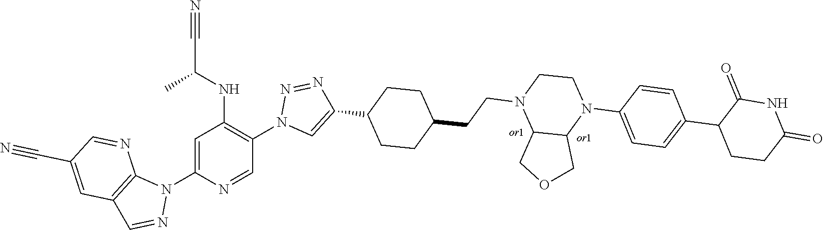 |
| P-238 | 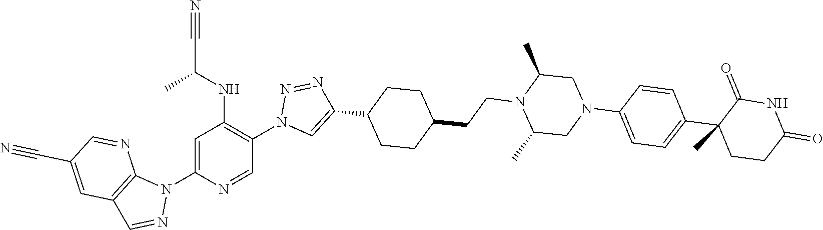 |
| P-239 | 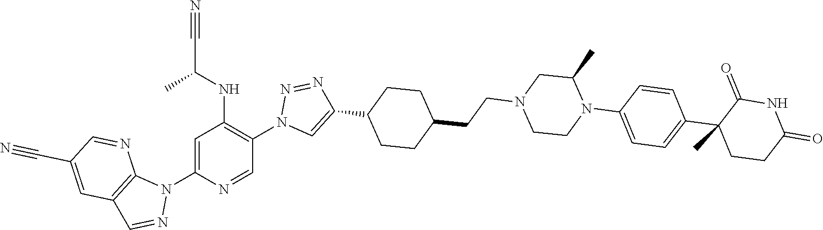 |
| P-240 | 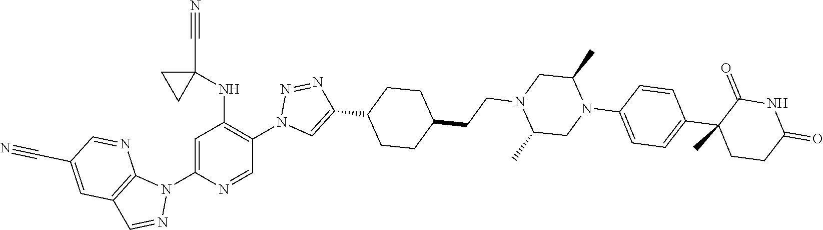 |
| P-241 | 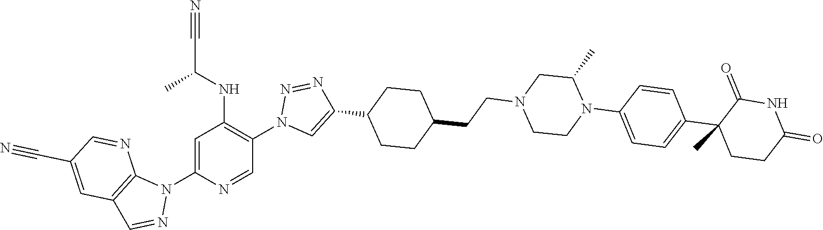 |
| P-242 | 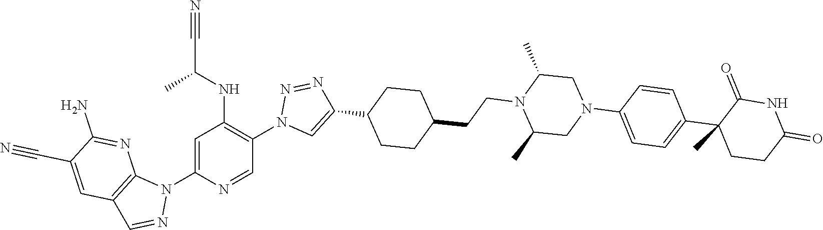 |
| P-243 | 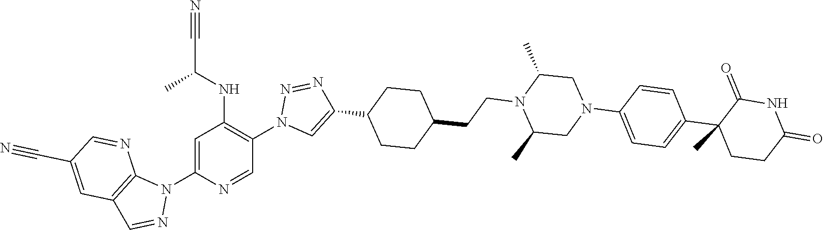 |
| P-244 | 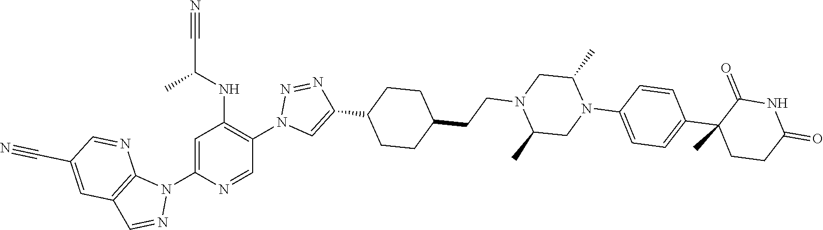 |
| P-245 | 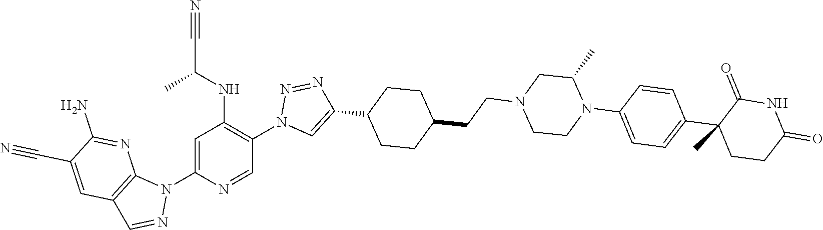 |
| P-246 | 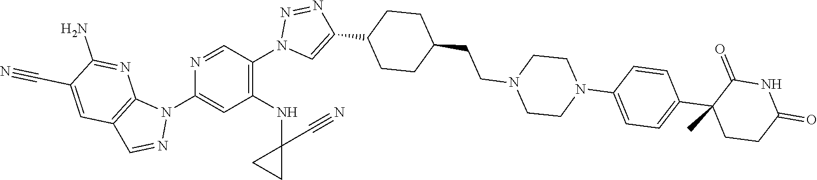 |
| P-247 | 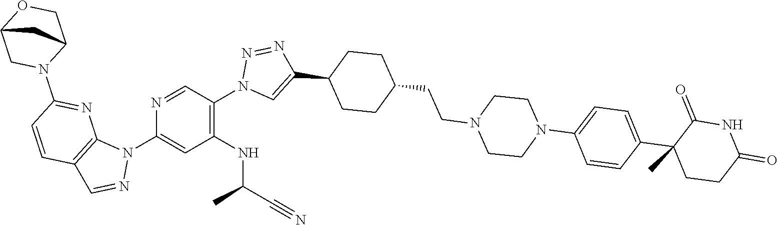 [SMILES: C[C@H](C#N)Nc1cc(-n2ncc3ccc(N4C[C@H]5C[C@@H]4CO5)nc32)ncc1-n1cc([C@H]2CC[C@H](CCN3CCN(c4ccc([C@@]5(C)CCC(=O)NC5=O)cc4)CC3)CC2)nn1] |
| P-248 | 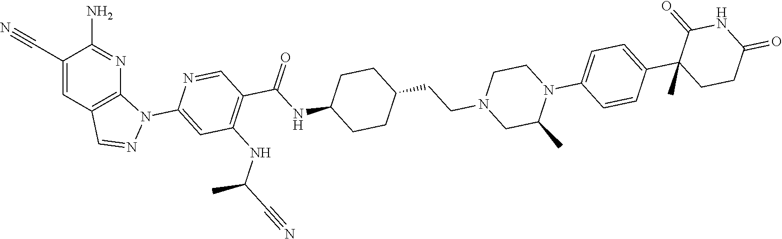 [SMILES: C[C@H](C#N)Nc1cc(-n2ncc3cc(C#N)c(N)nc32)ncc1C(=O)N[C@H]1CC[C@H](CCN2CCN(c3ccc([C@@]4(C)CCC(=O)NC4=O)cc3)[C@@H](C)C2)CC1] |
| P-249 | 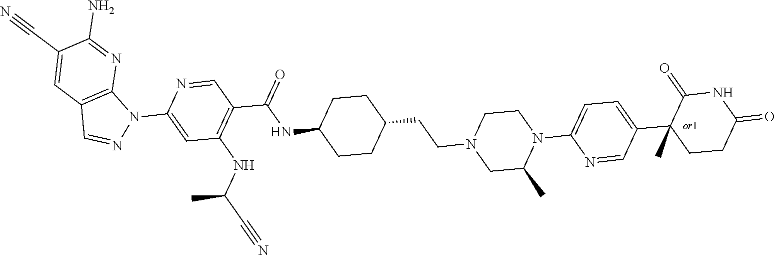 [SMILES: C[C@H](C#N)Nc1cc(-n2ncc3cc(C#N)c(N)nc32)ncc1C(=O)N[C@H]1CC[C@H](CCN2CCN(c3ccc([C@@]4(C)CCC(=O)NC4=O)cn3)[C@@H](C)C2)CC1] |
| P-250 | 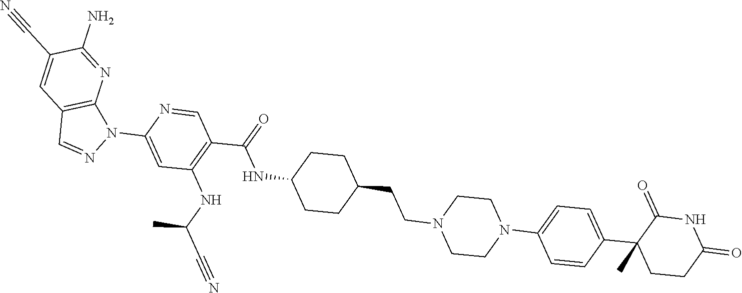 [SMILES: C[C@H](C#N)Nc1cc(-n2ncc3cc(C#N)c(N)nc32)ncc1C(=O)N[C@H]1CC[C@H](CCN2CCN(c3ccc([C@@]4(C)CCC(=O)NC4=O)cc3)CC2)CC1] |
| P-251 | 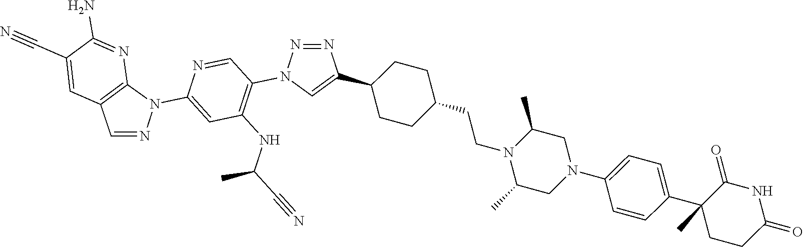 [SMILES: C[C@H](C#N)Nc1cc(-n2ncc3cc(C#N)c(N)nc32)ncc1-n1cc([C@H]2CC[C@H](CCN3[C@@H](C)CN(c4ccc([C@@]5(C)CCC(=O)NC5=O)cc4)C[C@@H]3C)CC2)nn1] |
| P-252 | 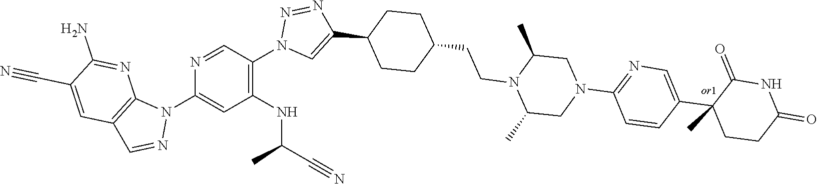 [SMILES: C[C@H](C#N)Nc1cc(-n2ncc3cc(C#N)c(N)nc32)ncc1-n1cc([C@H]2CC[C@H](CCN3[C@@H](C)CN(c4ccc([C@@]5(C)CCC(=O)NC5=O)cn4)C[C@@H]3C)CC2)nn1] |
| P-253 | 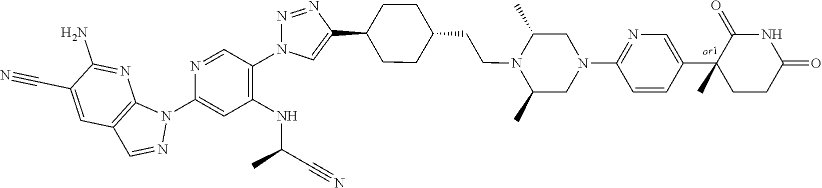 [SMILES: C[C@H](C#N)Nc1cc(-n2ncc3cc(C#N)c(N)nc32)ncc1-n1cc([C@H]2CC[C@H](CCN3[C@H](C)CN(c4ccc([C@@]5(C)CCC(=O)NC5=O)cn4)C[C@H]3C)CC2)nn1] |
| P-254 | 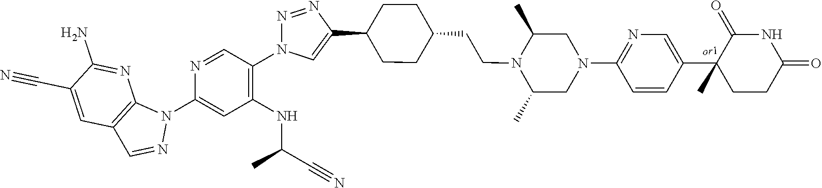 [SMILES: C[C@H](C#N)Nc1cc(-n2ncc3cc(C#N)c(N)nc32)ncc1-n1cc([C@H]2CC[C@H](CCN3[C@@H](C)CN(c4ccc([C@@]5(C)CCC(=O)NC5=O)cn4)C[C@@H]3C)CC2)nn1] |
| P-255 | 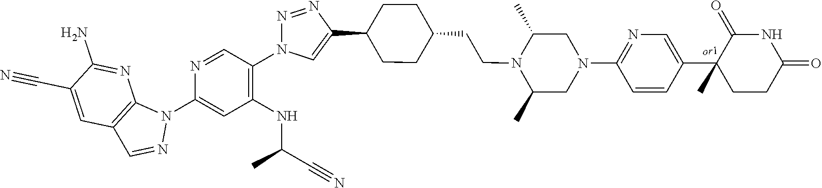 [SMILES: C[C@H](C#N)Nc1cc(-n2ncc3cc(C#N)c(N)nc32)ncc1-n1cc([C@H]2CC[C@H](CCN3[C@H](C)CN(c4ccc([C@@]5(C)CCC(=O)NC5=O)cn4)C[C@H]3C)CC2)nn1] |
| P-256 | 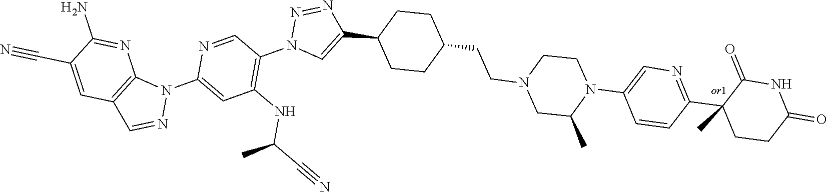 [SMILES: C[C@H](C#N)Nc1cc(-n2ncc3cc(C#N)c(N)nc32)ncc1-n1cc([C@H]2CC[C@H](CCN3CCN(c4ccc([C@@]5(C)CCC(=O)NC5=O)nc4)[C@@H](C)C3)CC2)nn1] |
| P-257 | 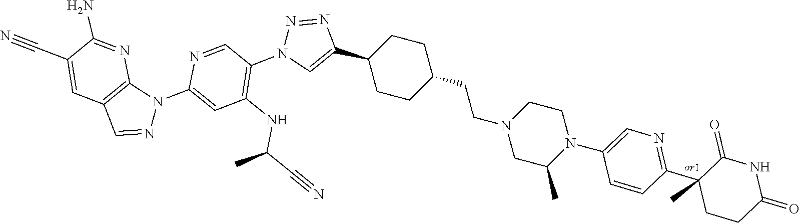 [SMILES: C[C@H](C#N)Nc1cc(-n2ncc3cc(C#N)c(N)nc32)ncc1-n1cc([C@H]2CC[C@H](CCN3CCN(c4ccc([C@@]5(C)CCC(=O)NC5=O)nc4)[C@@H](C)C3)CC2)nn1] |
| P-258 | 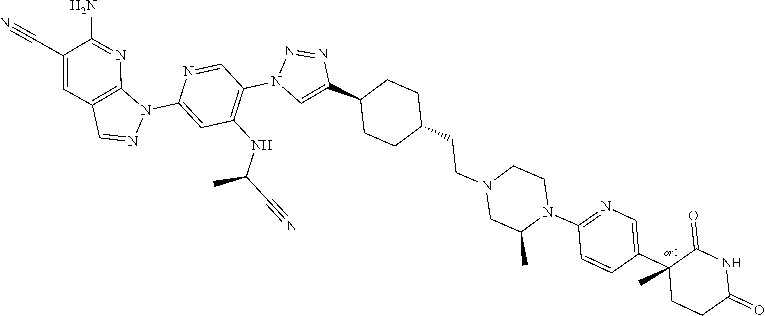 [SMILES: C[C@H](C#N)Nc1cc(-n2ncc3cc(C#N)c(N)nc32)ncc1-n1cc([C@H]2CC[C@H](CCN3CCN(c4ccc([C@@]5(C)CCC(=O)NC5=O)cn4)[C@@H](C)C3)CC2)nn1] |
| P-259 | 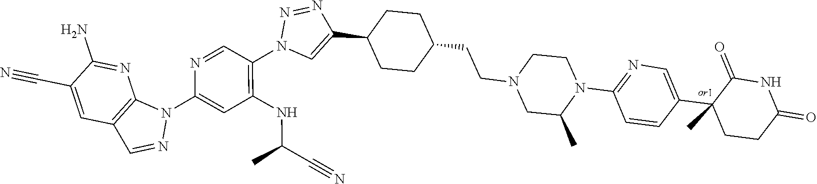 [SMILES: C[C@H](C#N)Nc1cc(-n2ncc3cc(C#N)c(N)nc32)ncc1-n1cc([C@H]2CC[C@H](CCN3CCN(c4ccc([C@@]5(C)CCC(=O)NC5=O)cn4)[C@@H](C)C3)CC2)nn1] |
| P-260 | 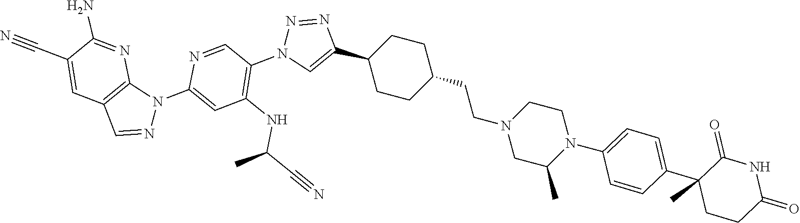 [SMILES: C[C@H](C#N)Nc1cc(-n2ncc3cc(C#N)c(N)nc32)ncc1-n1cc([C@H]2CC[C@H](CCN3CCN(c4ccc([C@@]5(C)CCC(=O)NC5=O)cc4)[C@@H](C)C3)CC2)nn1] |
| P-261 | 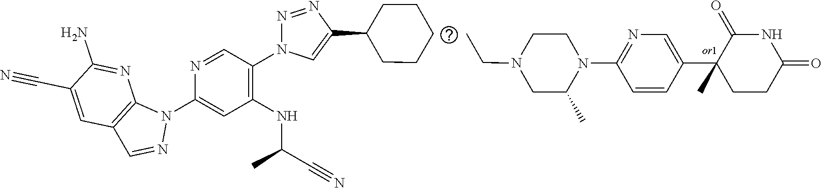 |
| P-262 | 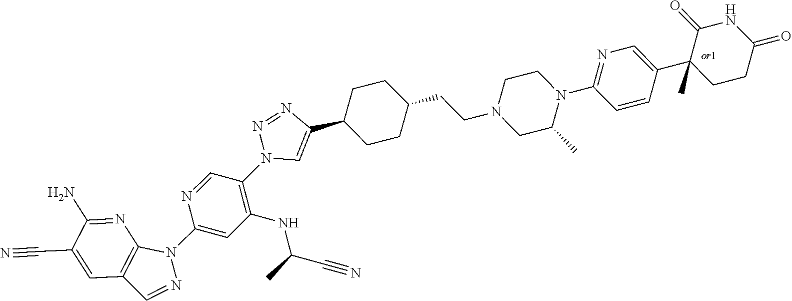 [SMILES: C[C@H](C#N)Nc1cc(-n2ncc3cc(C#N)c(N)nc32)ncc1-n1cc([C@H]2CC[C@H](CCN3CCN(c4ccc([C@@]5(C)CCC(=O)NC5=O)cn4)[C@H](C)C3)CC2)nn1] |
| P-263 | 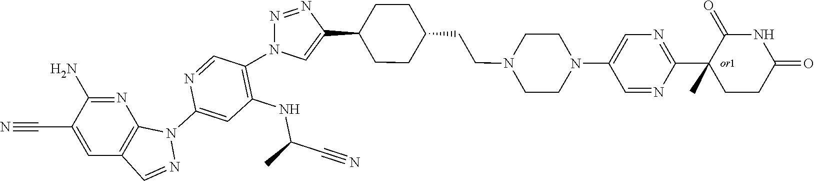 [SMILES: C[C@H](C#N)Nc1cc(-n2ncc3cc(C#N)c(N)nc32)ncc1-n1cc([C@H]2CC[C@H](CCN3CCN(c4cnc([C@@]5(C)CCC(=O)NC5=O)nc4)CC3)CC2)nn1] |
| P-264 | 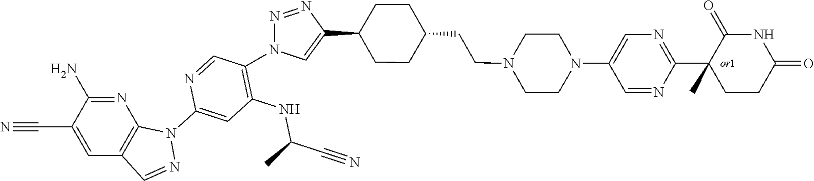 [SMILES: C[C@H](C#N)Nc1cc(-n2ncc3cc(C#N)c(N)nc32)ncc1-n1cc([C@H]2CC[C@H](CCN3CCN(c4cnc([C@@]5(C)CCC(=O)NC5=O)nc4)CC3)CC2)nn1] |
| P-265 | 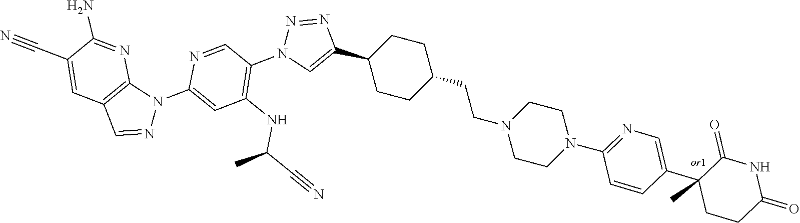 [SMILES: C[C@H](C#N)Nc1cc(-n2ncc3cc(C#N)c(N)nc32)ncc1-n1cc([C@H]2CC[C@H](CCN3CCN(c4ccc([C@@]5(C)CCC(=O)NC5=O)cn4)CC3)CC2)nn1] |
| P-266 | 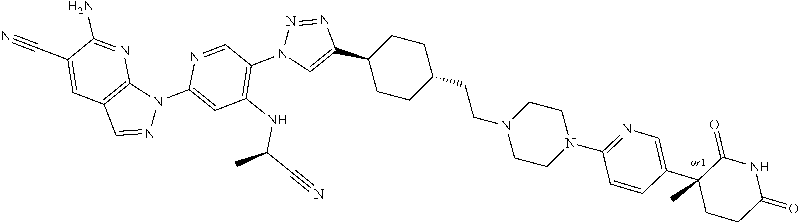 [SMILES: C[C@H](C#N)Nc1cc(-n2ncc3cc(C#N)c(N)nc32)ncc1-n1cc([C@H]2CC[C@H](CCN3CCN(c4ccc([C@@]5(C)CCC(=O)NC5=O)cn4)CC3)CC2)nn1] |
| P-267 | 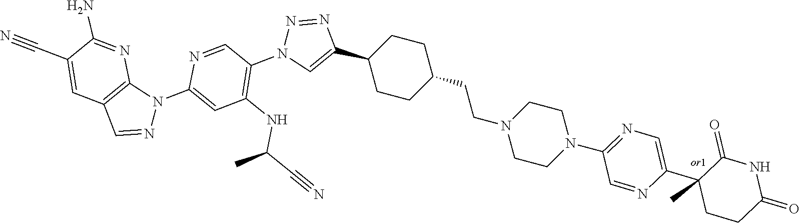 [SMILES: C[C@H](C#N)Nc1cc(-n2ncc3cc(C#N)c(N)nc32)ncc1-n1cc([C@H]2CC[C@H](CCN3CCN(c4cnc([C@@]5(C)CCC(=O)NC5=O)cn4)CC3)CC2)nn1] |
| P-268 | 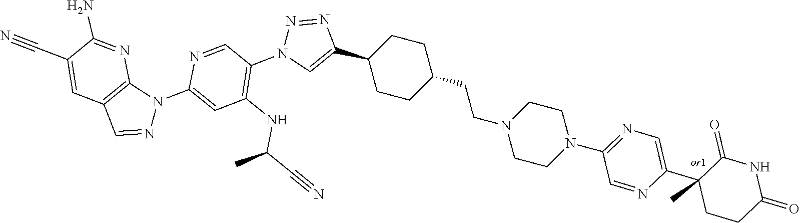 [SMILES: C[C@H](C#N)Nc1cc(-n2ncc3cc(C#N)c(N)nc32)ncc1-n1cc([C@H]2CC[C@H](CCN3CCN(c4cnc([C@@]5(C)CCC(=O)NC5=O)cn4)CC3)CC2)nn1] |
| P-269 | 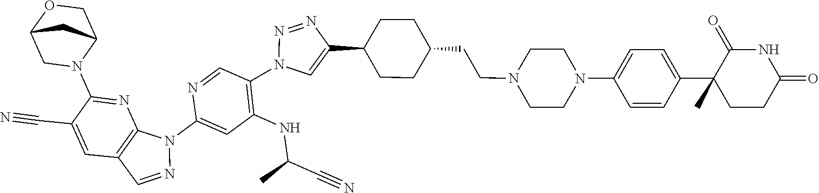 [SMILES: C[C@H](C#N)Nc1cc(-n2ncc3cc(C#N)c(N4C[C@H]5C[C@@H]4CO5)nc32)ncc1-n1cc([C@H]2CC[C@H](CCN3CCN(c4ccc([C@@]5(C)CCC(=O)NC5=O)cc4)CC3)CC2)nn1] |
| P-270 | 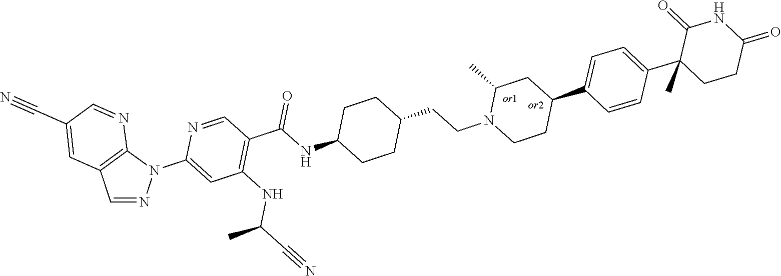 [SMILES: C[C@H](C#N)Nc1cc(-n2ncc3cc(C#N)cnc32)ncc1C(=O)N[C@H]1CC[C@H](CCN2CC[C@H](c3ccc([C@@]4(C)CCC(=O)NC4=O)cc3)C[C@H]2C)CC1] |
| P-271 | 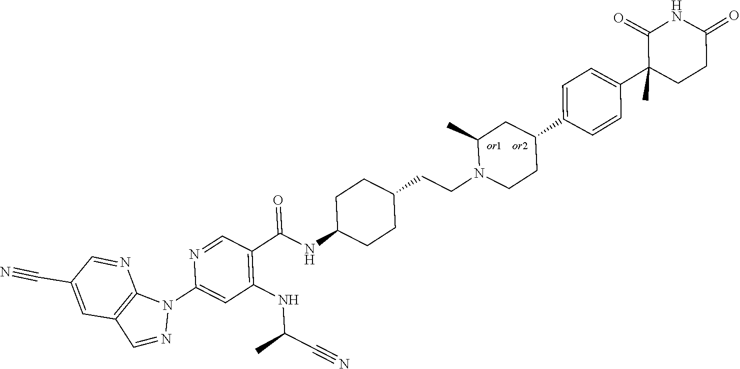 [SMILES: C[C@H](C#N)Nc1cc(-n2ncc3cc(C#N)cnc32)ncc1C(=O)N[C@H]1CC[C@H](CCN2CC[C@@H](c3ccc([C@@]4(C)CCC(=O)NC4=O)cc3)C[C@@H]2C)CC1] |
| P-272 | 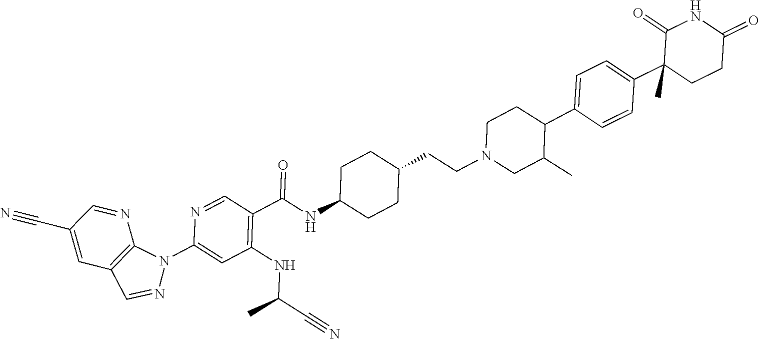 [SMILES: CC1CN(CC[C@H]2CC[C@H](NC(=O)c3cnc(-n4ncc5cc(C#N)cnc54)cc3N[C@H](C)C#N)CC2)CCC1c1ccc([C@@]2(C)CCC(=O)NC2=O)cc1] |
| P-273 | 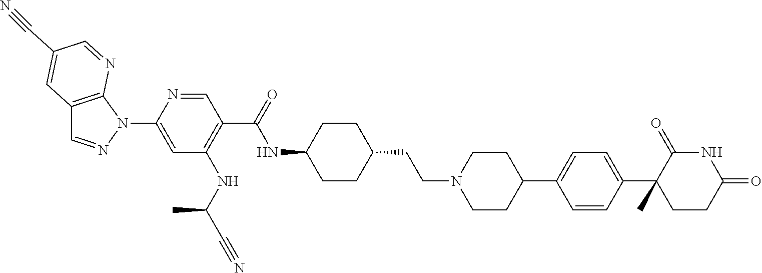 [SMILES: C[C@H](C#N)Nc1cc(-n2ncc3cc(C#N)cnc32)ncc1C(=O)N[C@H]1CC[C@H](CCN2CCC(c3ccc([C@@]4(C)CCC(=O)NC4=O)cc3)CC2)CC1] |
| P-274 | 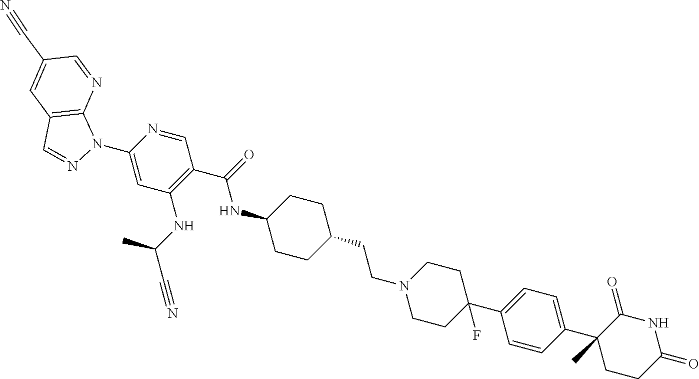 [SMILES: C[C@H](C#N)Nc1cc(-n2ncc3cc(C#N)cnc32)ncc1C(=O)N[C@H]1CC[C@H](CCN2CCC(F)(c3ccc([C@@]4(C)CCC(=O)NC4=O)cc3)CC2)CC1] |
| P-275 | 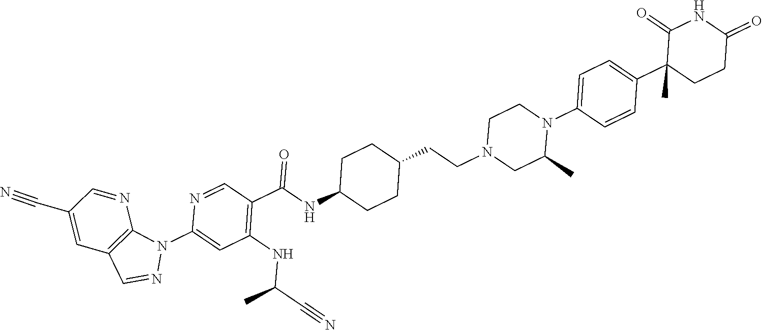 [SMILES: C[C@H](C#N)Nc1cc(-n2ncc3cc(C#N)cnc32)ncc1C(=O)N[C@H]1CC[C@H](CCN2CCN(c3ccc([C@@]4(C)CCC(=O)NC4=O)cc3)[C@@H](C)C2)CC1] |
| P-276 | 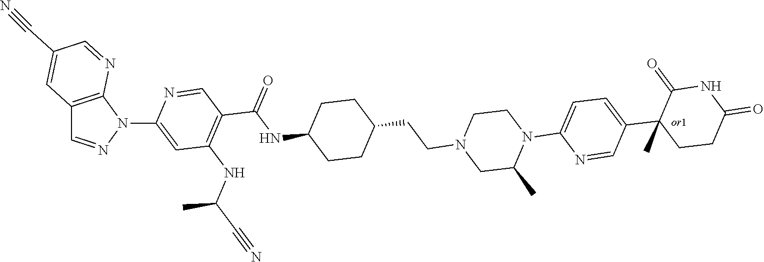 [SMILES: C[C@H](C#N)Nc1cc(-n2ncc3cc(C#N)cnc32)ncc1C(=O)N[C@H]1CC[C@H](CCN2CCN(c3ccc([C@@]4(C)CCC(=O)NC4=O)cn3)[C@@H](C)C2)CC1] |
| P-277 | 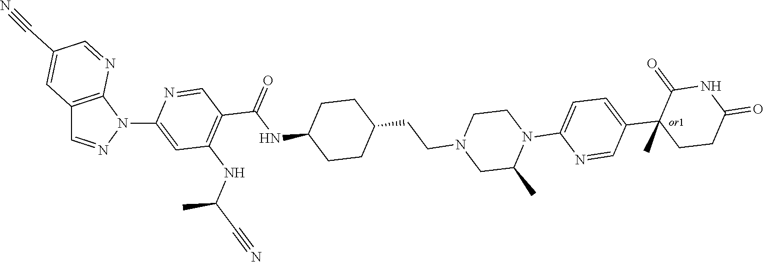 [SMILES: C[C@H](C#N)Nc1cc(-n2ncc3cc(C#N)cnc32)ncc1C(=O)N[C@H]1CC[C@H](CCN2CCN(c3ccc([C@@]4(C)CCC(=O)NC4=O)cn3)[C@@H](C)C2)CC1] |
| P-278 | 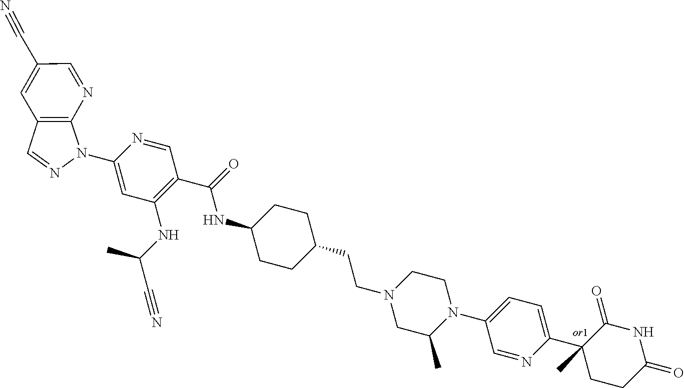 [SMILES: C[C@H](C#N)Nc1cc(-n2ncc3cc(C#N)cnc32)ncc1C(=O)N[C@H]1CC[C@H](CCN2CCN(c3ccc([C@@]4(C)CCC(=O)NC4=O)nc3)[C@@H](C)C2)CC1] |
| P-279 | 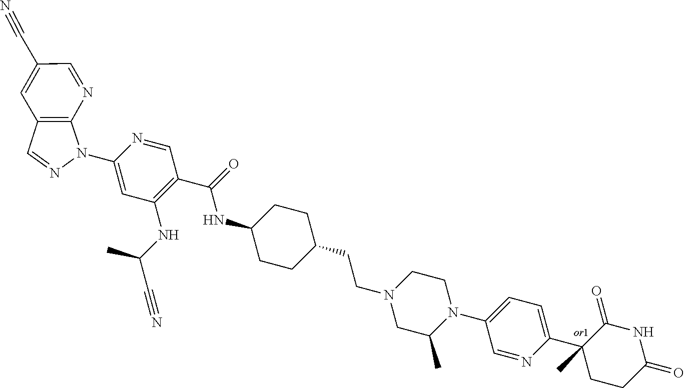 [SMILES: C[C@H](C#N)Nc1cc(-n2ncc3cc(C#N)cnc32)ncc1C(=O)N[C@H]1CC[C@H](CCN2CCN(c3ccc([C@@]4(C)CCC(=O)NC4=O)nc3)[C@@H](C)C2)CC1] |
| P-280 | 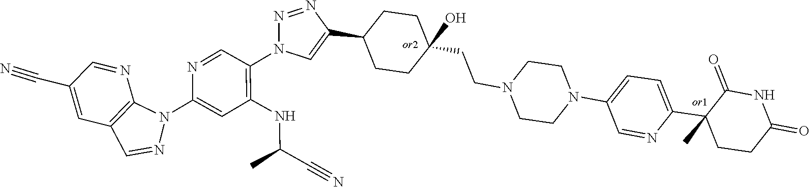 [SMILES: C[C@H](C#N)Nc1cc(-n2ncc3cc(C#N)cnc32)ncc1-n1cc([C@H]2CC[C@](O)(CCN3CCN(c4ccc([C@@]5(C)CCC(=O)NC5=O)nc4)CC3)CC2)nn1] |
| P-281 | 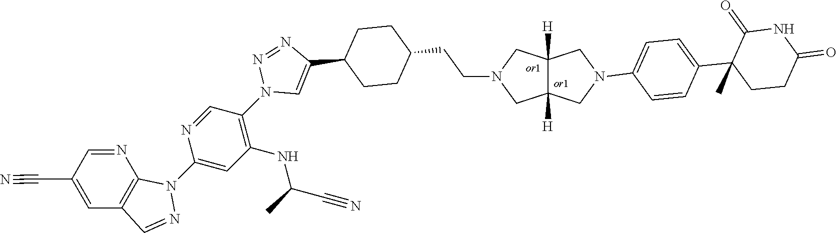 [SMILES: C[C@H](C#N)Nc1cc(-n2ncc3cc(C#N)cnc32)ncc1-n1cc([C@H]2CC[C@H](CCN3C[C@@H]4CN(c5ccc([C@@]6(C)CCC(=O)NC6=O)cc5)C[C@@H]4C3)CC2)nn1] |
| P-282 | 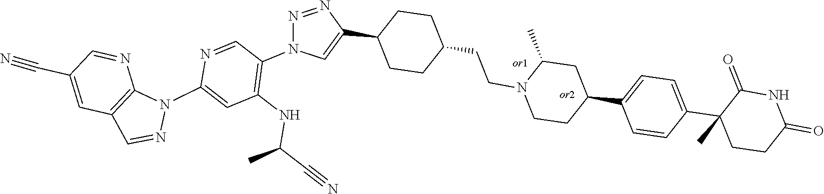 [SMILES: C[C@H](C#N)Nc1cc(-n2ncc3cc(C#N)cnc32)ncc1-n1cc([C@H]2CC[C@H](CCN3CC[C@H](c4ccc([C@@]5(C)CCC(=O)NC5=O)cc4)C[C@H]3C)CC2)nn1] |
| P-283 | 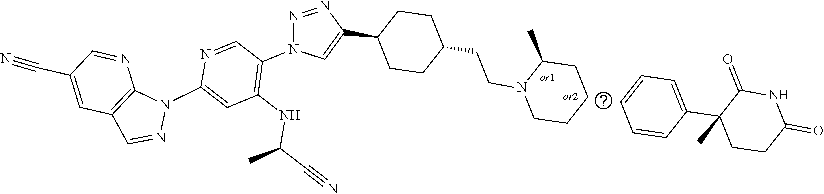 |
| P-284 | 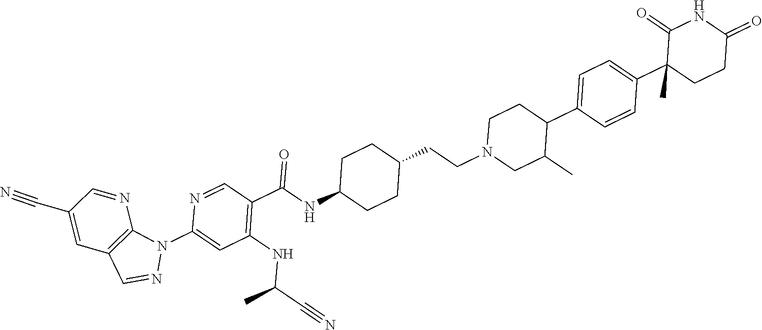 [SMILES: CC1CN(CC[C@H]2CC[C@H](NC(=O)c3cnc(-n4ncc5cc(C#N)cnc54)cc3N[C@H](C)C#N)CC2)CCC1c1ccc([C@@]2(C)CCC(=O)NC2=O)cc1] |
| P-285 | 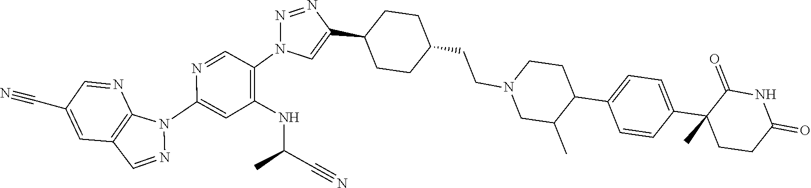 [SMILES: CC1CN(CC[C@H]2CC[C@H](c3cn(-c4cnc(-n5ncc6cc(C#N)cnc65)cc4N[C@H](C)C#N)nn3)CC2)CCC1c1ccc([C@@]2(C)CCC(=O)NC2=O)cc1] |
| P-286 | 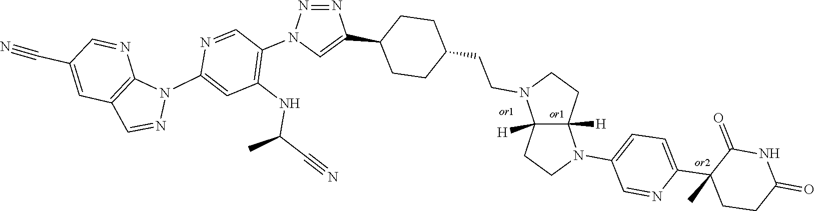 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc([C@@]4(C)CCC(=O)NC4=O)nc3)CC2)nn1] |
| P-287 | 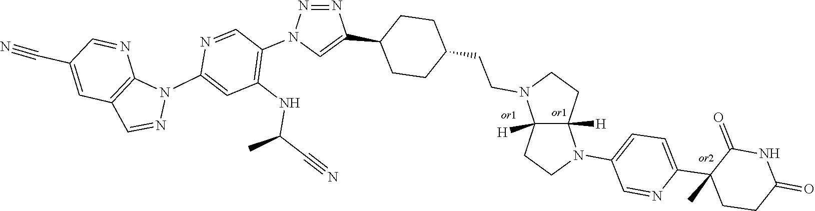 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc([C@@]4(C)CCC(=O)NC4=O)nc3)CC2)nn1] |
| P-288 | 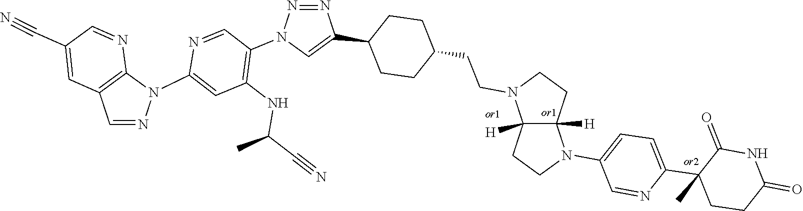 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc([C@@]4(C)CCC(=O)NC4=O)nc3)CC2)nn1] |
| P-289 | 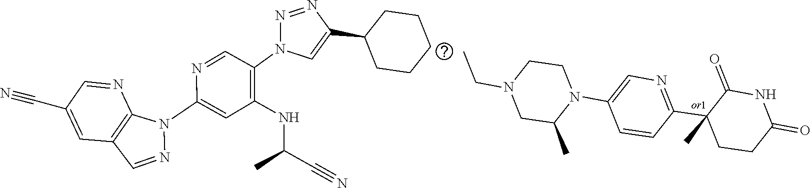 |
| P-290 | 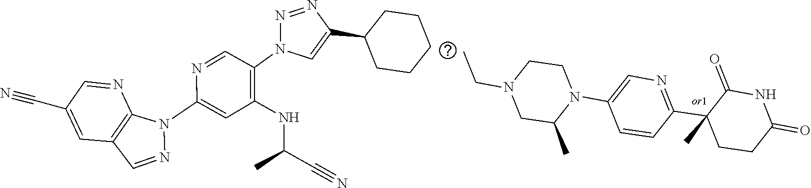 |
| P-291 | 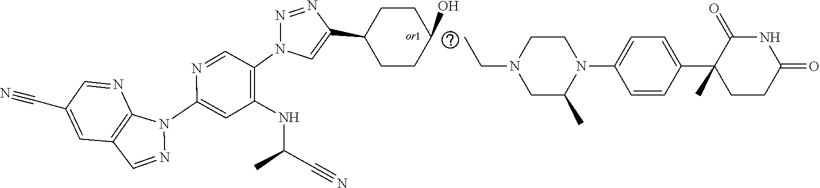 |
| P-292 | 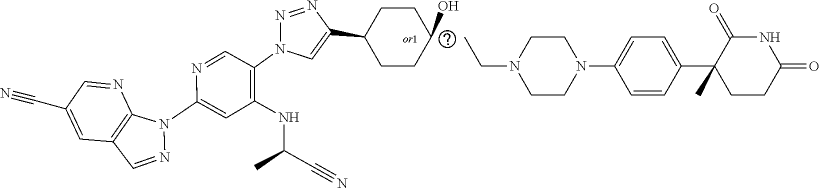 |
| P-293 | 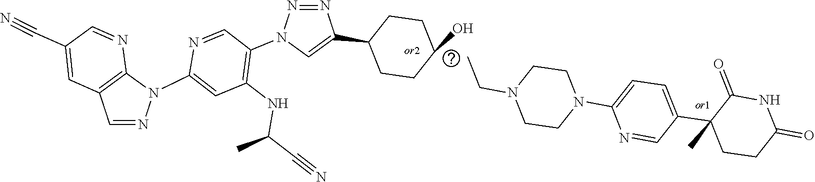 |
| P-294 | 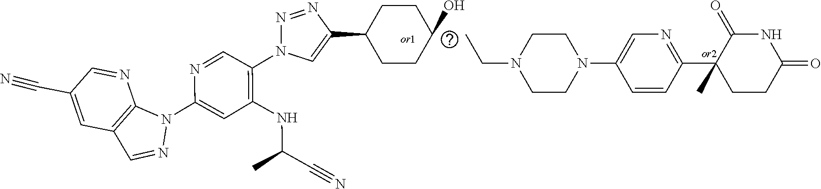 |
| P-295 | 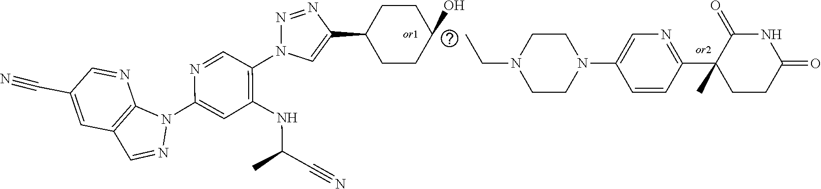 |
| P-296 | 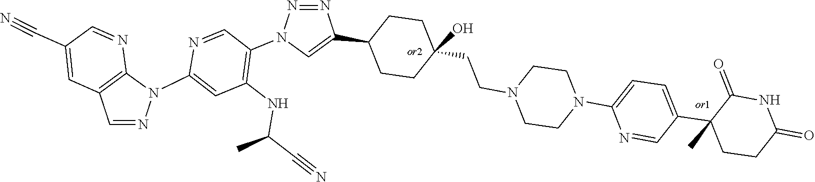 [SMILES: C[C@H](C#N)Nc1cc(-n2ncc3cc(C#N)cnc32)ncc1-n1cc([C@H]2CC[C@](O)(CCN3CCN(c4ccc([C@@]5(C)CCC(=O)NC5=O)cn4)CC3)CC2)nn1] |
| P-297 | 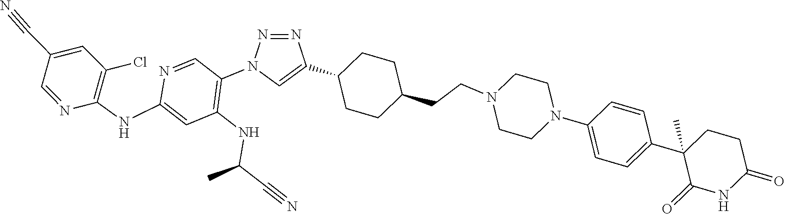 [SMILES: C[C@H](C#N)Nc1cc(Nc2ncc(C#N)cc2Cl)ncc1-n1cc([C@H]2CC[C@H](CCN3CCN(c4ccc([C@@]5(C)CCC(=O)NC5=O)cc4)CC3)CC2)nn1] |
| P-298 | 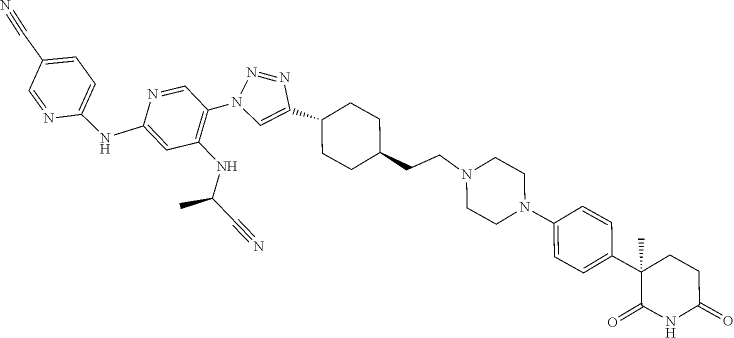 [SMILES: C[C@H](C#N)Nc1cc(Nc2ccc(C#N)cn2)ncc1-n1cc([C@H]2CC[C@H](CCN3CCN(c4ccc([C@@]5(C)CCC(=O)NC5=O)cc4)CC3)CC2)nn1] |
| P-299 | 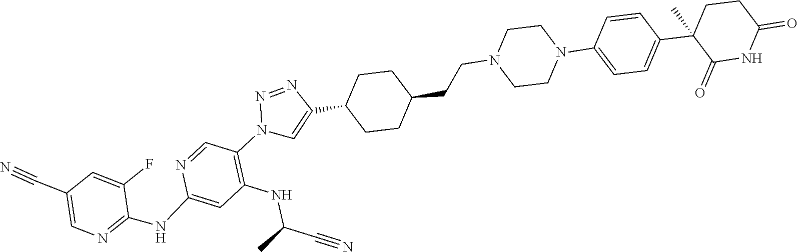 [SMILES: C[C@H](C#N)Nc1cc(Nc2ncc(C#N)cc2F)ncc1-n1cc([C@H]2CC[C@H](CCN3CCN(c4ccc([C@@]5(C)CCC(=O)NC5=O)cc4)CC3)CC2)nn1] |
| P-300 | 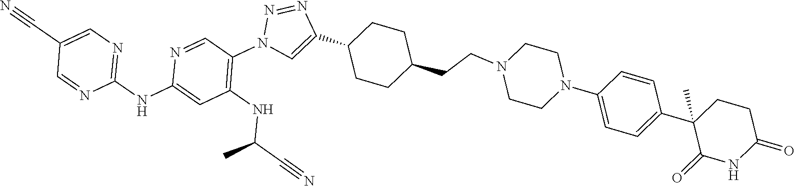 [SMILES: C[C@H](C#N)Nc1cc(Nc2ncc(C#N)cn2)ncc1-n1cc([C@H]2CC[C@H](CCN3CCN(c4ccc([C@@]5(C)CCC(=O)NC5=O)cc4)CC3)CC2)nn1] |
| P-301 | 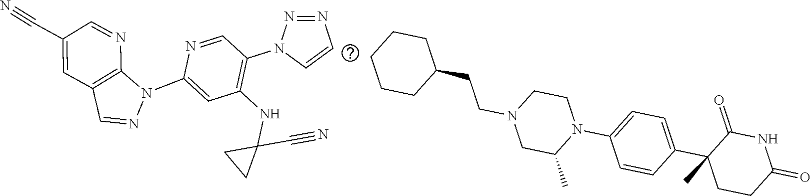 |
| P-302 | 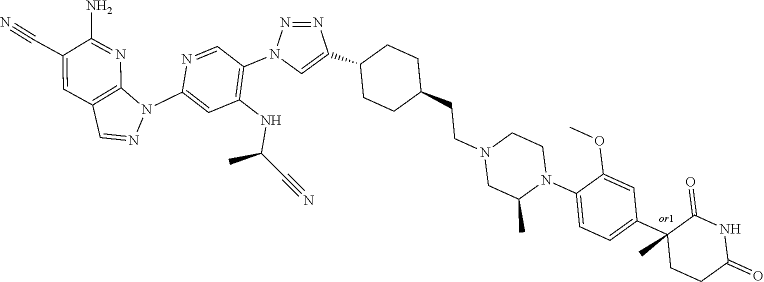 [SMILES: COc1cc([C@@]2(C)CCC(=O)NC2=O)ccc1N1CCN(CC[C@H]2CC[C@H](c3cn(-c4cnc(-n5ncc6cc(C#N)c(N)nc65)cc4N[C@H](C)C#N)nn3)CC2)C[C@@H]1C] |
| P-303 | 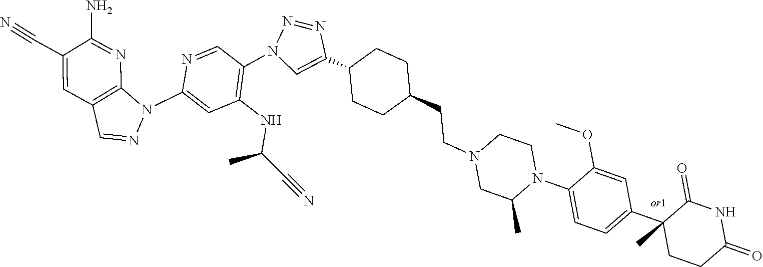 [SMILES: COc1cc([C@@]2(C)CCC(=O)NC2=O)ccc1N1CCN(CC[C@H]2CC[C@H](c3cn(-c4cnc(-n5ncc6cc(C#N)c(N)nc65)cc4N[C@H](C)C#N)nn3)CC2)C[C@@H]1C] |
| P-304 | 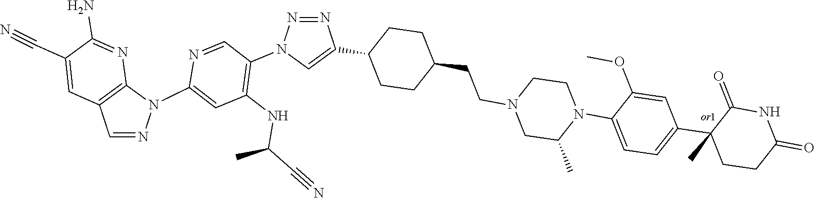 [SMILES: COc1cc([C@@]2(C)CCC(=O)NC2=O)ccc1N1CCN(CC[C@H]2CC[C@H](c3cn(-c4cnc(-n5ncc6cc(C#N)c(N)nc65)cc4N[C@H](C)C#N)nn3)CC2)C[C@H]1C] |
| P-305 | 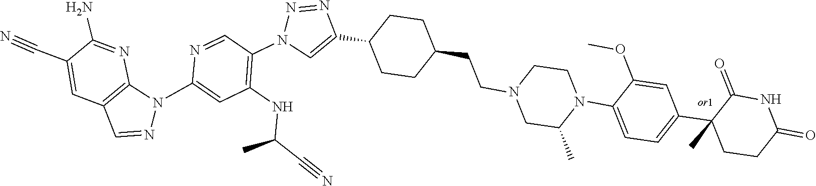 [SMILES: COc1cc([C@@]2(C)CCC(=O)NC2=O)ccc1N1CCN(CC[C@H]2CC[C@H](c3cn(-c4cnc(-n5ncc6cc(C#N)c(N)nc65)cc4N[C@H](C)C#N)nn3)CC2)C[C@H]1C] |
| P-306 | 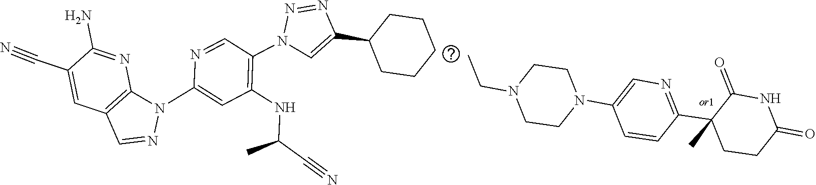 |
| P-307 | 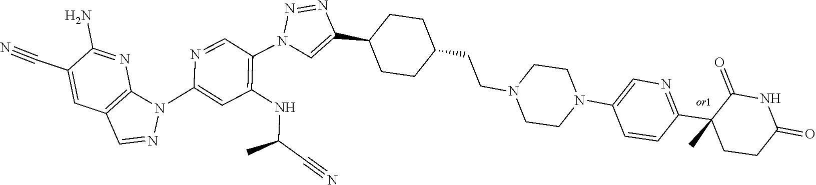 [SMILES: C[C@H](C#N)Nc1cc(-n2ncc3cc(C#N)c(N)nc32)ncc1-n1cc([C@H]2CC[C@H](CCN3CCN(c4ccc([C@@]5(C)CCC(=O)NC5=O)nc4)CC3)CC2)nn1] |
| P-308 | 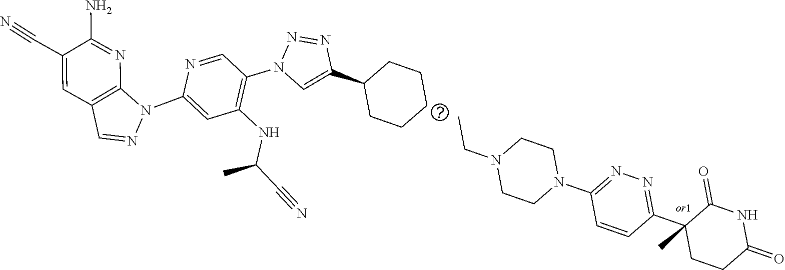 |
| P-309 | 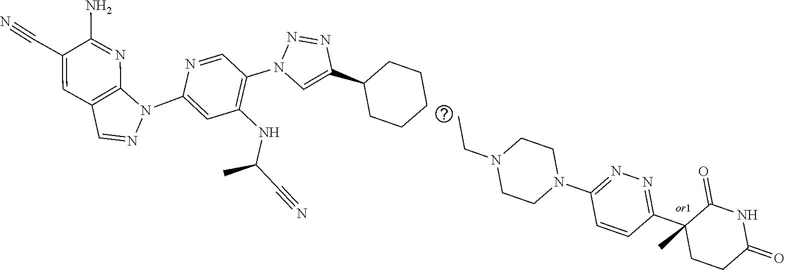 |
| P-310 | 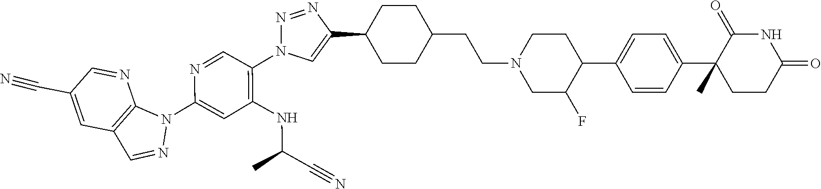 |
| P-311 | 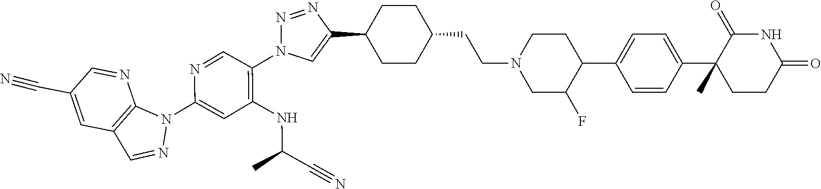 [SMILES: C[C@H](C#N)Nc1cc(-n2ncc3cc(C#N)cnc32)ncc1-n1cc([C@H]2CC[C@H](CCN3CCC(c4ccc([C@@]5(C)CCC(=O)NC5=O)cc4)C(F)C3)CC2)nn1] |
| P-312 | 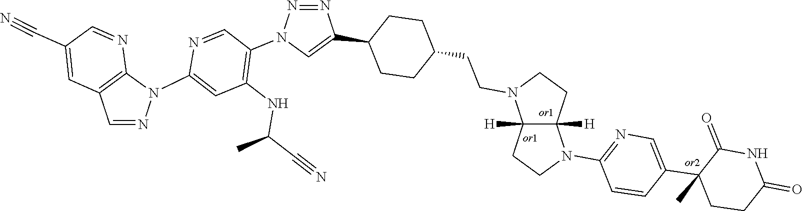 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc([C@@]4(C)CCC(=O)NC4=O)cn3)CC2)nn1] |
| P-313 | 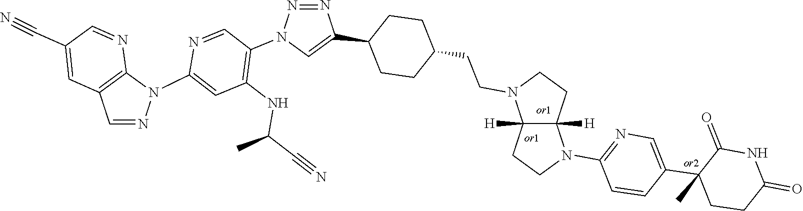 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc([C@@]4(C)CCC(=O)NC4=O)cn3)CC2)nn1] |
| P-314 | 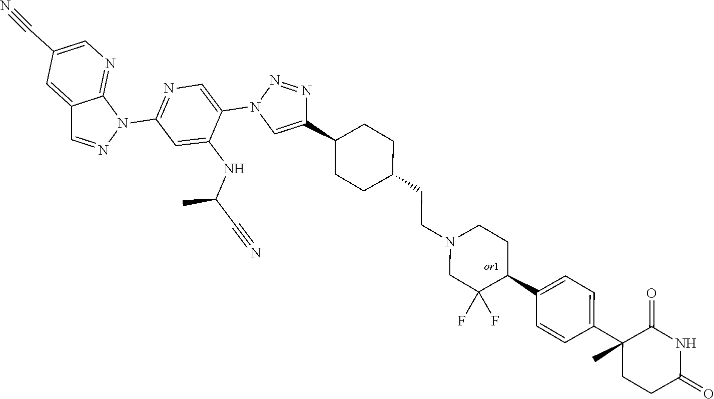 [SMILES: C[C@H](C#N)Nc1cc(-n2ncc3cc(C#N)cnc32)ncc1-n1cc([C@H]2CC[C@H](CCN3CC[C@@H](c4ccc([C@@]5(C)CCC(=O)NC5=O)cc4)C(F)(F)C3)CC2)nn1] |
| P-315 | 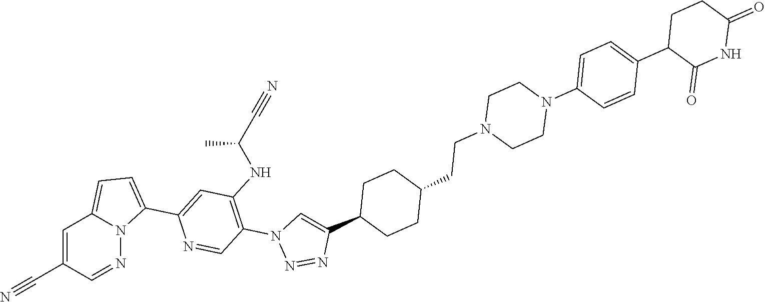 [SMILES: C[C@H](C#N)Nc1cc(-c2ccc3cc(C#N)cnn23)ncc1-n1cc([C@H]2CC[C@H](CCN3CCN(c4ccc(C5CCC(=O)NC5=O)cc4)CC3)CC2)nn1] |
| P-316 | 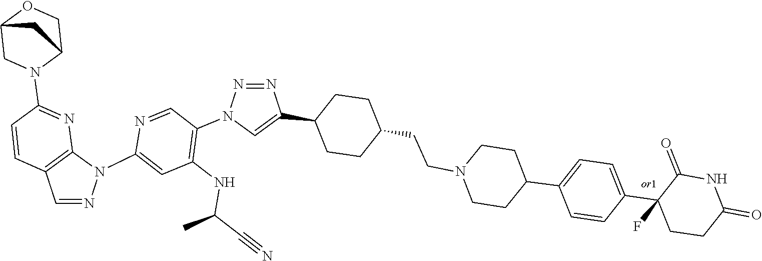 [SMILES: C[C@H](C#N)Nc1cc(-n2ncc3ccc(N4C[C@H]5C[C@@H]4CO5)nc32)ncc1-n1cc([C@H]2CC[C@H](CCN3CCC(c4ccc([C@@]5(F)CCC(=O)NC5=O)cc4)CC3)CC2)nn1] |
| P-317 | 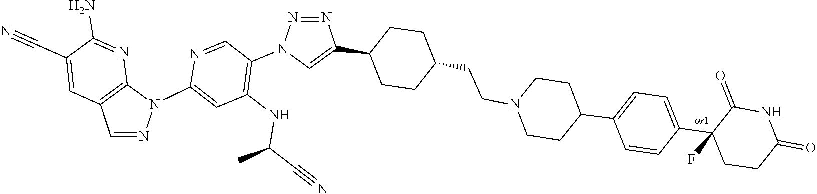 [SMILES: C[C@H](C#N)Nc1cc(-n2ncc3cc(C#N)c(N)nc32)ncc1-n1cc([C@H]2CC[C@H](CCN3CCC(c4ccc([C@@]5(F)CCC(=O)NC5=O)cc4)CC3)CC2)nn1] |
| P-318 | 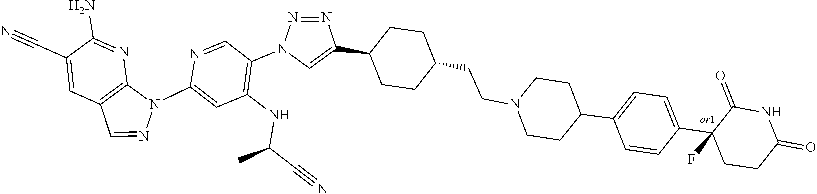 [SMILES: C[C@H](C#N)Nc1cc(-n2ncc3cc(C#N)c(N)nc32)ncc1-n1cc([C@H]2CC[C@H](CCN3CCC(c4ccc([C@@]5(F)CCC(=O)NC5=O)cc4)CC3)CC2)nn1] |
| P-319 | 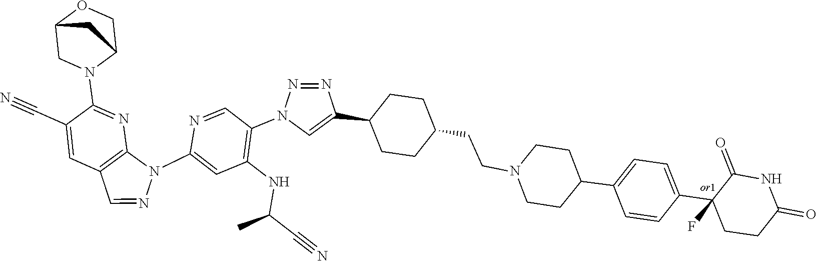 [SMILES: C[C@H](C#N)Nc1cc(-n2ncc3cc(C#N)c(N4C[C@H]5C[C@@H]4CO5)nc32)ncc1-n1cc([C@H]2CC[C@H](CCN3CCC(c4ccc([C@@]5(F)CCC(=O)NC5=O)cc4)CC3)CC2)nn1] |
| P-320 | 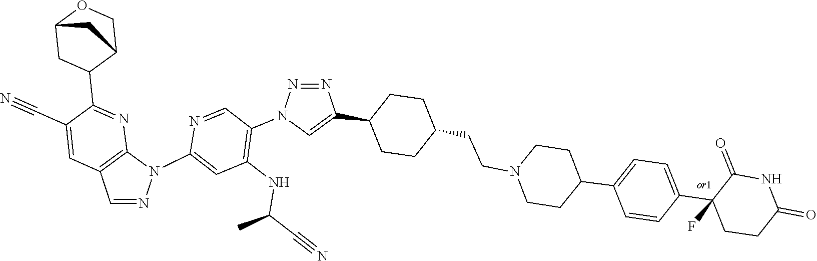 [SMILES: C[C@H](C#N)Nc1cc(-n2ncc3cc(C#N)c(C4C[C@H]5C[C@@H]4CO5)nc32)ncc1-n1cc([C@H]2CC[C@H](CCN3CCC(c4ccc([C@@]5(F)CCC(=O)NC5=O)cc4)CC3)CC2)nn1] |
| P-321 | 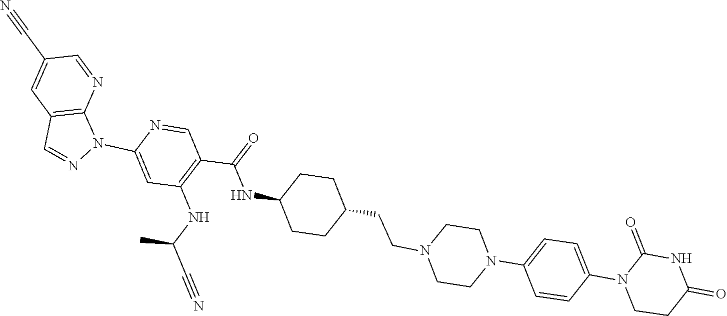 [SMILES: C[C@H](C#N)Nc1cc(-n2ncc3cc(C#N)cnc32)ncc1C(=O)N[C@H]1CC[C@H](CCN2CCN(c3ccc(N4CCC(=O)NC4=O)cc3)CC2)CC1] |
| P-322 | 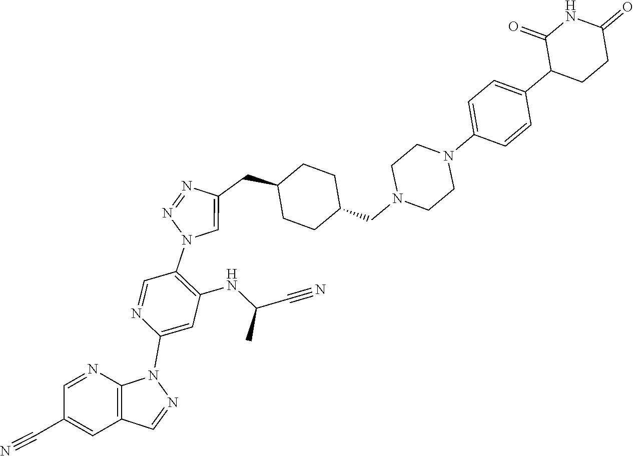 [SMILES: C[C@H](C#N)Nc1cc(-n2ncc3cc(C#N)cnc32)ncc1-n1cc(C[C@H]2CC[C@H](CN3CCN(c4ccc(C5CCC(=O)NC5=O)cc4)CC3)CC2)nn1] |
| P-323 | 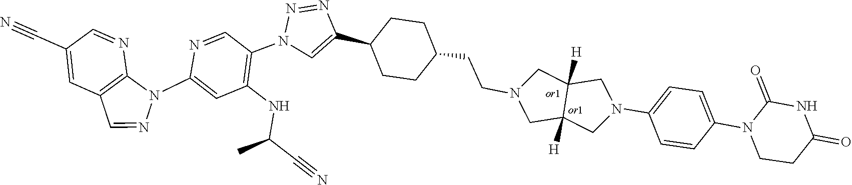 [SMILES: C[C@H](C#N)Nc1cc(-n2ncc3cc(C#N)cnc32)ncc1-n1cc([C@H]2CC[C@H](CCN3C[C@@H]4CN(c5ccc(N6CCC(=O)NC6=O)cc5)C[C@@H]4C3)CC2)nn1] |
| P-324 | 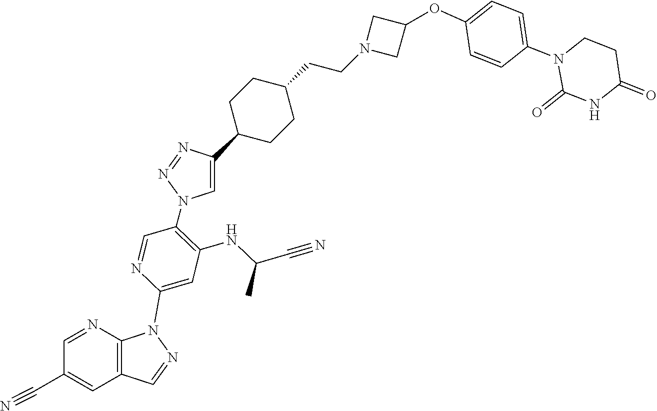 [SMILES: C[C@H](C#N)Nc1cc(-n2ncc3cc(C#N)cnc32)ncc1-n1cc([C@H]2CC[C@H](CCN3CC(Oc4ccc(N5CCC(=O)NC5=O)cc4)C3)CC2)nn1] |
| P-325 | 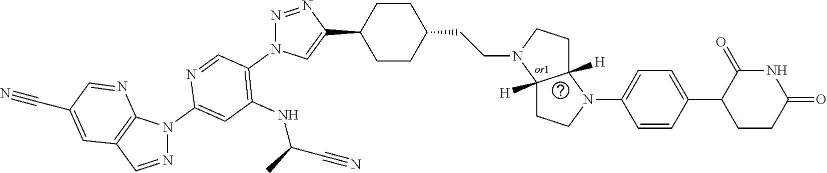 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc(C4CCC(=O)NC4=O)cc3)CC2)nn1] |
| P-326 | 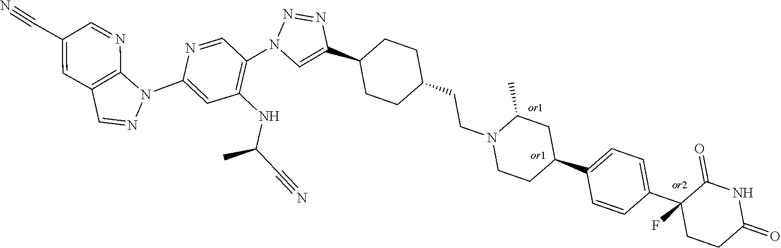 [SMILES: C[C@H](C#N)Nc1cc(-n2ncc3cc(C#N)cnc32)ncc1-n1cc([C@H]2CC[C@H](CCN3CC[C@H](c4ccc([C@@]5(F)CCC(=O)NC5=O)cc4)C[C@H]3C)CC2)nn1] |
| P-327 | 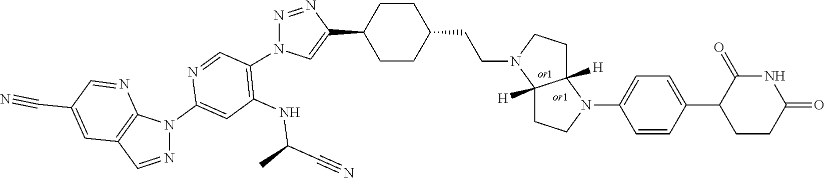 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc(C4CCC(=O)NC4=O)cc3)CC2)nn1] |
| P-328 | 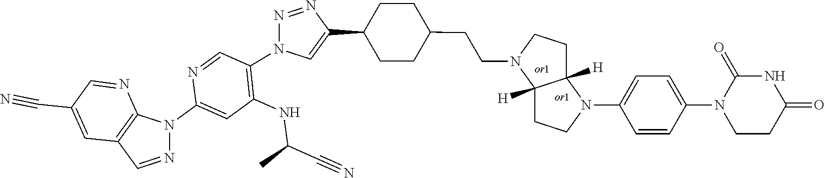 |
| P-329 | 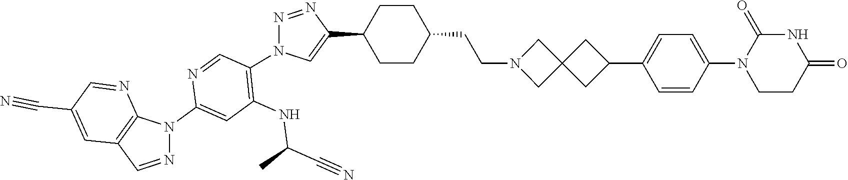 [SMILES: C[C@H](C#N)Nc1cc(-n2ncc3cc(C#N)cnc32)ncc1-n1cc([C@H]2CC[C@H](CCN3CC4(CC(c5ccc(N6CCC(=O)NC6=O)cc5)C4)C3)CC2)nn1] |
| P-330 | 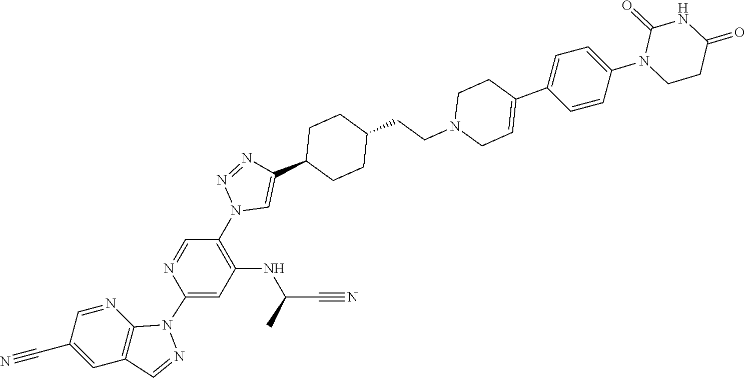 [SMILES: C[C@H](C#N)Nc1cc(-n2ncc3cc(C#N)cnc32)ncc1-n1cc([C@H]2CC[C@H](CCN3CC=C(c4ccc(N5CCC(=O)NC5=O)cc4)CC3)CC2)nn1] |
| P-331 | 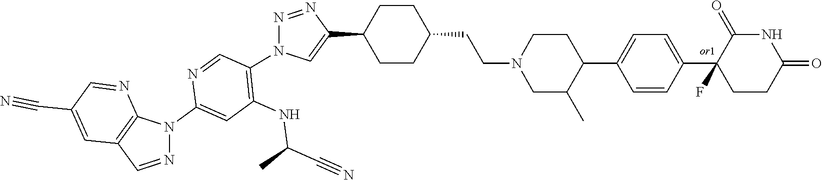 [SMILES: CC1CN(CC[C@H]2CC[C@H](c3cn(-c4cnc(-n5ncc6cc(C#N)cnc65)cc4N[C@H](C)C#N)nn3)CC2)CCC1c1ccc([C@@]2(F)CCC(=O)NC2=O)cc1] |
| P-332 | 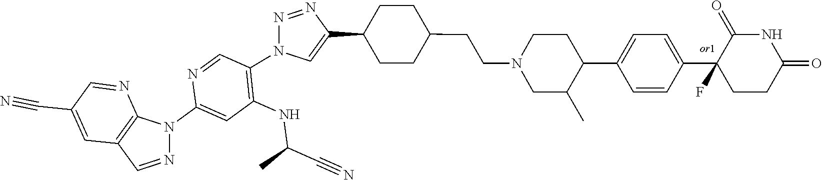 |
| P-333 | 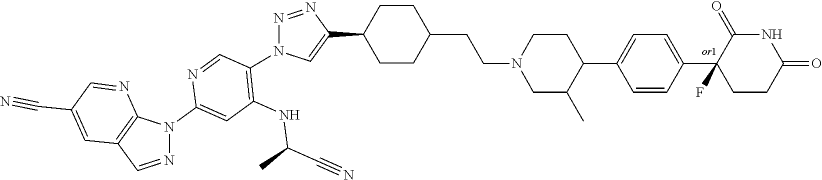 |
| P-334 | 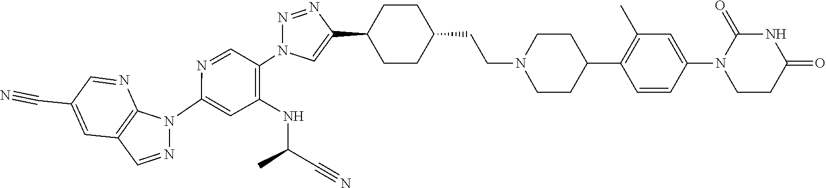 [SMILES: Cc1cc(N2CCC(=O)NC2=O)ccc1C1CCN(CC[C@H]2CC[C@H](c3cn(-c4cnc(-n5ncc6cc(C#N)cnc65)cc4N[C@H](C)C#N)nn3)CC2)CC1] |
| P-335 | 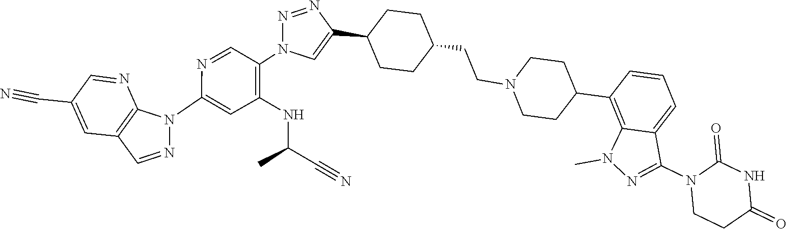 [SMILES: C[C@H](C#N)Nc1cc(-n2ncc3cc(C#N)cnc32)ncc1-n1cc([C@H]2CC[C@H](CCN3CCC(c4cccc5c(N6CCC(=O)NC6=O)nn(C)c45)CC3)CC2)nn1] |
| P-336 | 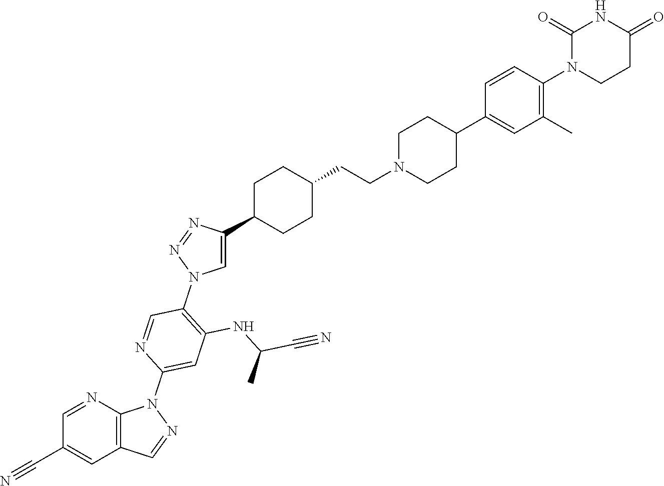 [SMILES: Cc1cc(C2CCN(CC[C@H]3CC[C@H](c4cn(-c5cnc(-n6ncc7cc(C#N)cnc76)cc5N[C@H](C)C#N)nn4)CC3)CC2)ccc1N1CCC(=O)NC1=O] |
| P-337 | 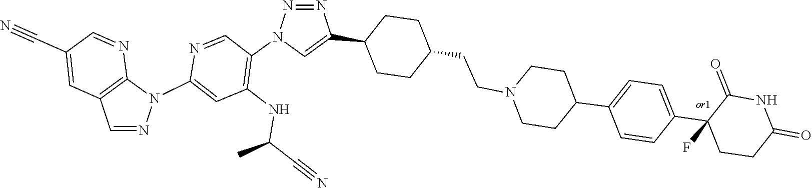 [SMILES: C[C@H](C#N)Nc1cc(-n2ncc3cc(C#N)cnc32)ncc1-n1cc([C@H]2CC[C@H](CCN3CCC(c4ccc([C@@]5(F)CCC(=O)NC5=O)cc4)CC3)CC2)nn1] |
| P-338 | 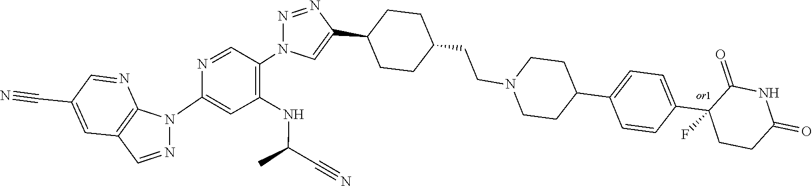 [SMILES: C[C@H](C#N)Nc1cc(-n2ncc3cc(C#N)cnc32)ncc1-n1cc([C@H]2CC[C@H](CCN3CCC(c4ccc([C@]5(F)CCC(=O)NC5=O)cc4)CC3)CC2)nn1] |
| P-339 | 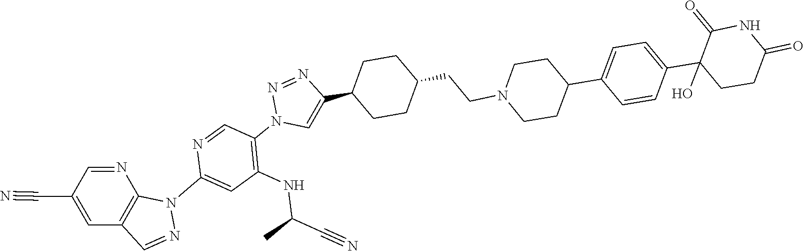 [SMILES: C[C@H](C#N)Nc1cc(-n2ncc3cc(C#N)cnc32)ncc1-n1cc([C@H]2CC[C@H](CCN3CCC(c4ccc(C5(O)CCC(=O)NC5=O)cc4)CC3)CC2)nn1] |
| P-340 | 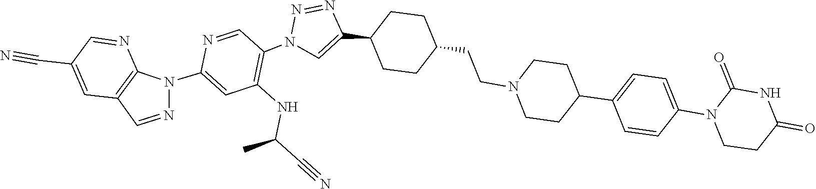 [SMILES: C[C@H](C#N)Nc1cc(-n2ncc3cc(C#N)cnc32)ncc1-n1cc([C@H]2CC[C@H](CCN3CCC(c4ccc(N5CCC(=O)NC5=O)cc4)CC3)CC2)nn1] |
| P-341 | 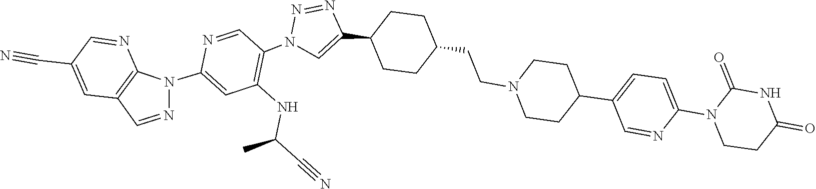 [SMILES: C[C@H](C#N)Nc1cc(-n2ncc3cc(C#N)cnc32)ncc1-n1cc([C@H]2CC[C@H](CCN3CCC(c4ccc(N5CCC(=O)NC5=O)nc4)CC3)CC2)nn1] |
| P-342 | 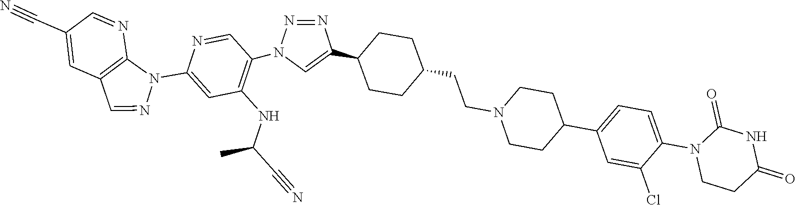 [SMILES: C[C@H](C#N)Nc1cc(-n2ncc3cc(C#N)cnc32)ncc1-n1cc([C@H]2CC[C@H](CCN3CCC(c4ccc(N5CCC(=O)NC5=O)c(Cl)c4)CC3)CC2)nn1] |
| P-343 | 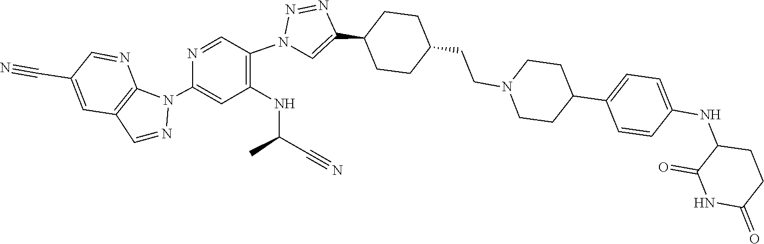 [SMILES: C[C@H](C#N)Nc1cc(-n2ncc3cc(C#N)cnc32)ncc1-n1cc([C@H]2CC[C@H](CCN3CCC(c4ccc(NC5CCC(=O)NC5=O)cc4)CC3)CC2)nn1] |
| P-344 | 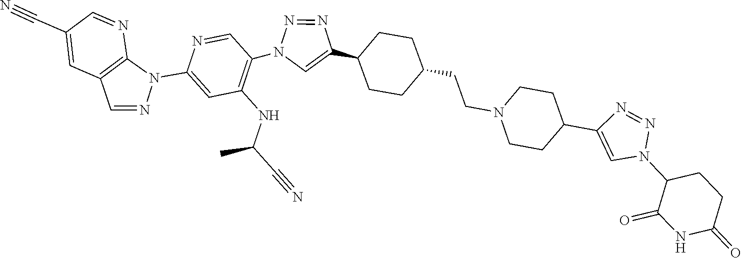 [SMILES: C[C@H](C#N)Nc1cc(-n2ncc3cc(C#N)cnc32)ncc1-n1cc([C@H]2CC[C@H](CCN3CCC(c4cn(C5CCC(=O)NC5=O)nn4)CC3)CC2)nn1] |
| P-345 | 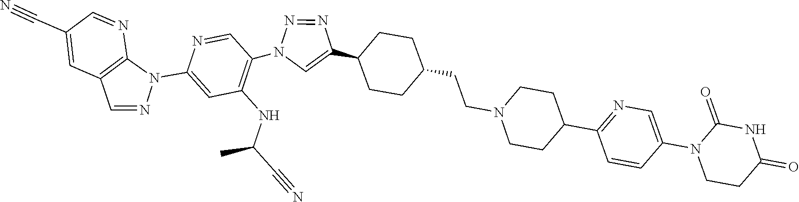 [SMILES: C[C@H](C#N)Nc1cc(-n2ncc3cc(C#N)cnc32)ncc1-n1cc([C@H]2CC[C@H](CCN3CCC(c4ccc(N5CCC(=O)NC5=O)cn4)CC3)CC2)nn1] |
| P-346 | 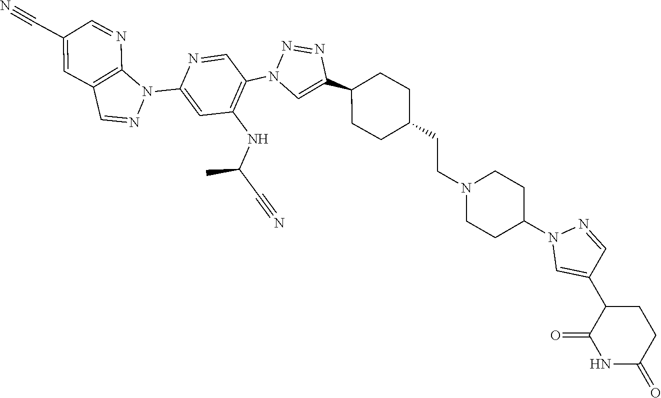 [SMILES: C[C@H](C#N)Nc1cc(-n2ncc3cc(C#N)cnc32)ncc1-n1cc([C@H]2CC[C@H](CCN3CCC(n4cc(C5CCC(=O)NC5=O)cn4)CC3)CC2)nn1] |
| P-347 | 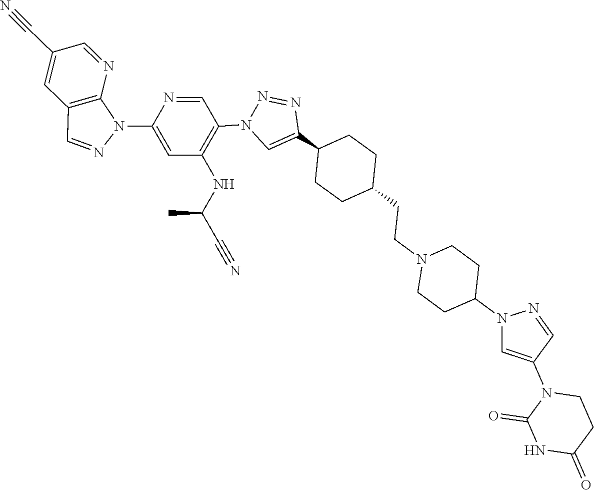 [SMILES: C[C@H](C#N)Nc1cc(-n2ncc3cc(C#N)cnc32)ncc1-n1cc([C@H]2CC[C@H](CCN3CCC(n4cc(N5CCC(=O)NC5=O)cn4)CC3)CC2)nn1] |
| P-348 | 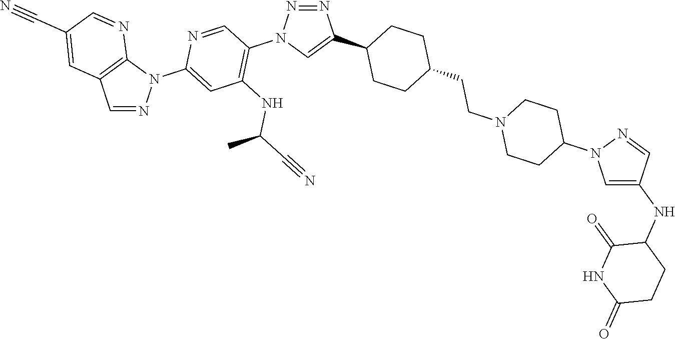 [SMILES: C[C@H](C#N)Nc1cc(-n2ncc3cc(C#N)cnc32)ncc1-n1cc([C@H]2CC[C@H](CCN3CCC(n4cc(NC5CCC(=O)NC5=O)cn4)CC3)CC2)nn1] |
| P-349 | 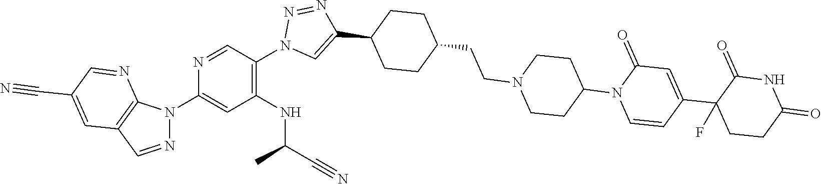 [SMILES: C[C@H](C#N)Nc1cc(-n2ncc3cc(C#N)cnc32)ncc1-n1cc([C@H]2CC[C@H](CCN3CCC(n4ccc(C5(F)CCC(=O)NC5=O)cc4=O)CC3)CC2)nn1] |
| P-350 | 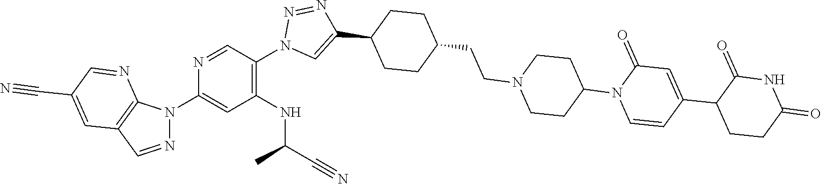 [SMILES: C[C@H](C#N)Nc1cc(-n2ncc3cc(C#N)cnc32)ncc1-n1cc([C@H]2CC[C@H](CCN3CCC(n4ccc(C5CCC(=O)NC5=O)cc4=O)CC3)CC2)nn1] |
| P-351 | 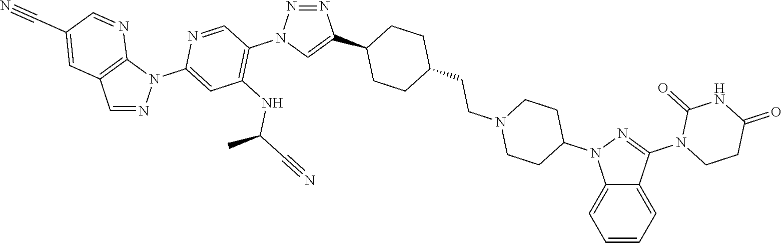 [SMILES: C[C@H](C#N)Nc1cc(-n2ncc3cc(C#N)cnc32)ncc1-n1cc([C@H]2CC[C@H](CCN3CCC(n4nc(N5CCC(=O)NC5=O)c5ccccc54)CC3)CC2)nn1] |
| P-352 | 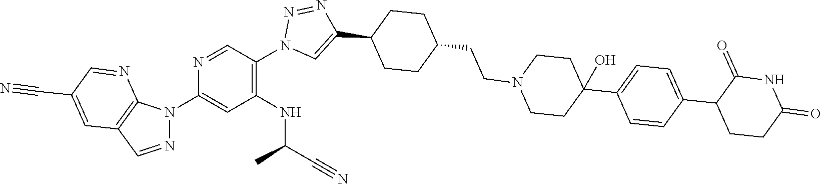 [SMILES: C[C@H](C#N)Nc1cc(-n2ncc3cc(C#N)cnc32)ncc1-n1cc([C@H]2CC[C@H](CCN3CCC(O)(c4ccc(C5CCC(=O)NC5=O)cc4)CC3)CC2)nn1] |
| P-353 | 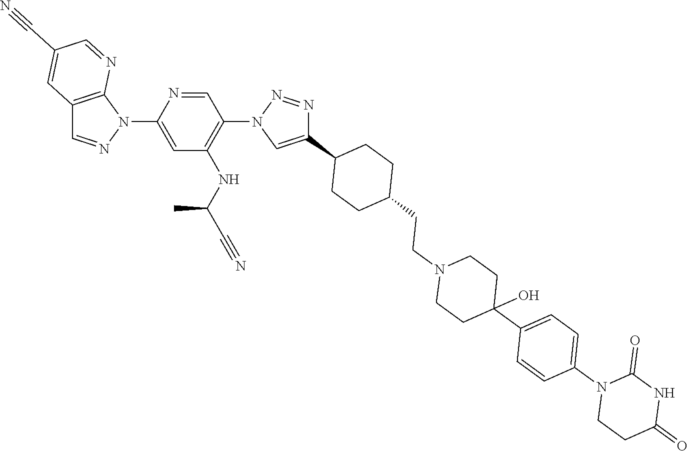 [SMILES: C[C@H](C#N)Nc1cc(-n2ncc3cc(C#N)cnc32)ncc1-n1cc([C@H]2CC[C@H](CCN3CCC(O)(c4ccc(N5CCC(=O)NC5=O)cc4)CC3)CC2)nn1] |
| P-354 | 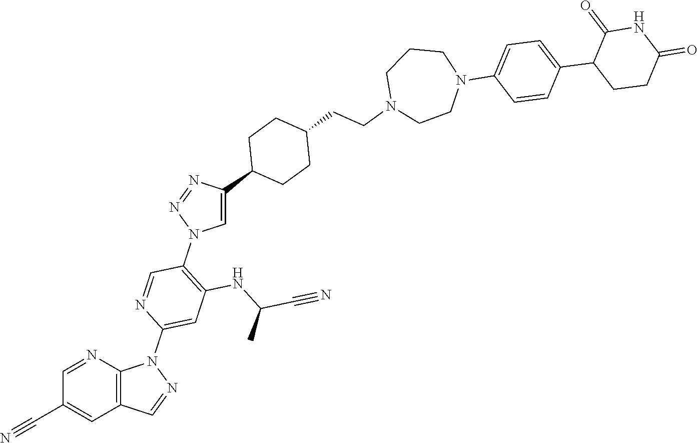 [SMILES: C[C@H](C#N)Nc1cc(-n2ncc3cc(C#N)cnc32)ncc1-n1cc([C@H]2CC[C@H](CCN3CCCN(c4ccc(C5CCC(=O)NC5=O)cc4)CC3)CC2)nn1] |
| P-355 | 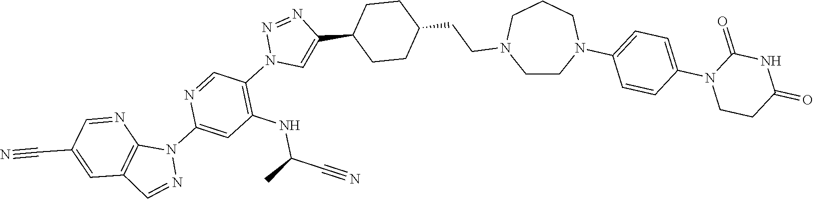 [SMILES: C[C@H](C#N)Nc1cc(-n2ncc3cc(C#N)cnc32)ncc1-n1cc([C@H]2CC[C@H](CCN3CCCN(c4ccc(N5CCC(=O)NC5=O)cc4)CC3)CC2)nn1] |
| P-356 | 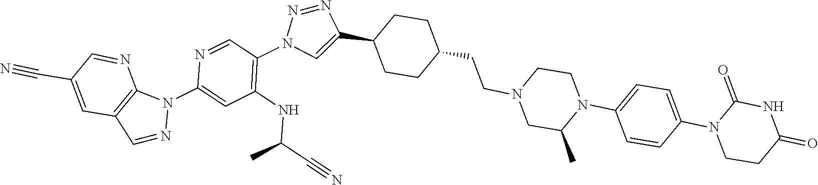 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)cc4)[C@@H](C)C3)CC2)nn1] |
| P-357 | 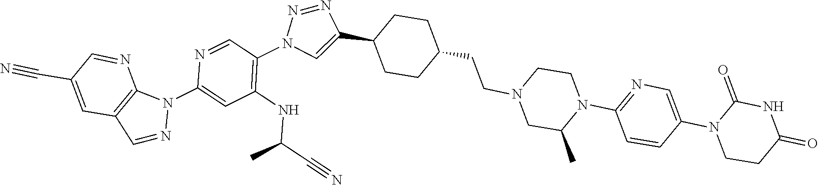 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)cn4)[C@@H](C)C3)CC2)nn1] |
| P-358 | 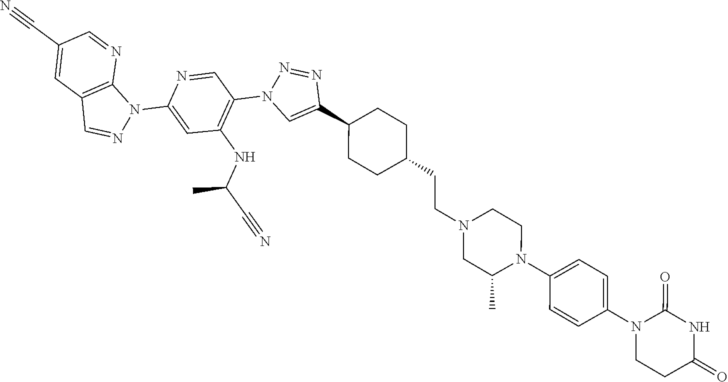 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)cc4)[C@H](C)C3)CC2)nn1] |
| P-359 | 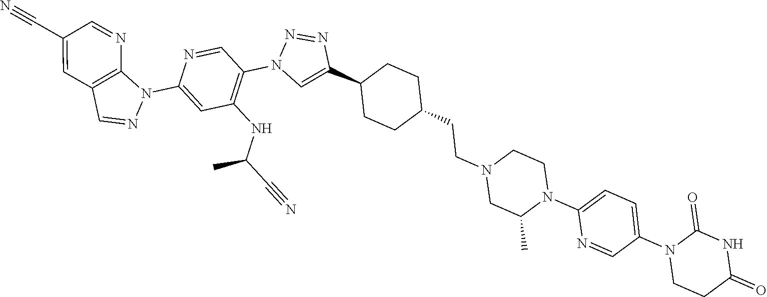 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)cn4)[C@H](C)C3)CC2)nn1] |
| P-360 | 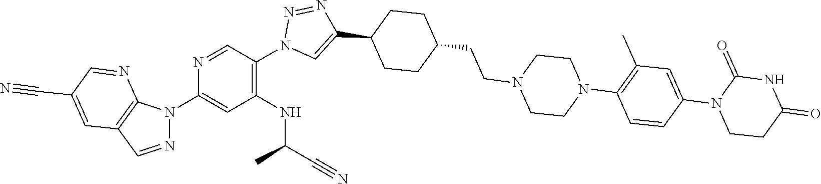 [SMILES: Cc1cc(N2CCC(=O)NC2=O)ccc1N1CCN(CC[C@H]2CC[C@H](c3cn(-c4cnc(-n5ncc6cc(C#N)cnc65)cc4N[C@H](C)C#N)nn3)CC2)CC1] |
| P-361 | 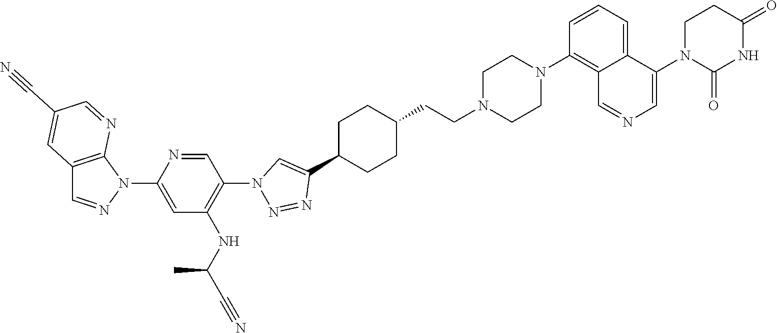 [SMILES: C[C@H](C#N)Nc1cc(-n2ncc3cc(C#N)cnc32)ncc1-n1cc([C@H]2CC[C@H](CCN3CCN(c4cccc5c(N6CCC(=O)NC6=O)cncc45)CC3)CC2)nn1] |
| P-362 | 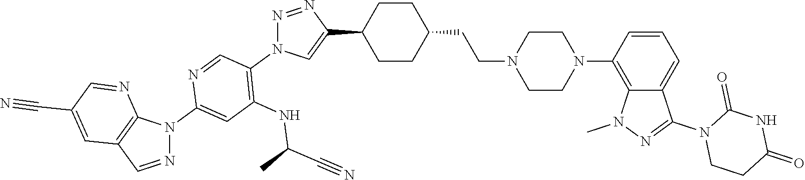 [SMILES: C[C@H](C#N)Nc1cc(-n2ncc3cc(C#N)cnc32)ncc1-n1cc([C@H]2CC[C@H](CCN3CCN(c4cccc5c(N6CCC(=O)NC6=O)nn(C)c45)CC3)CC2)nn1] |
| P-363 | 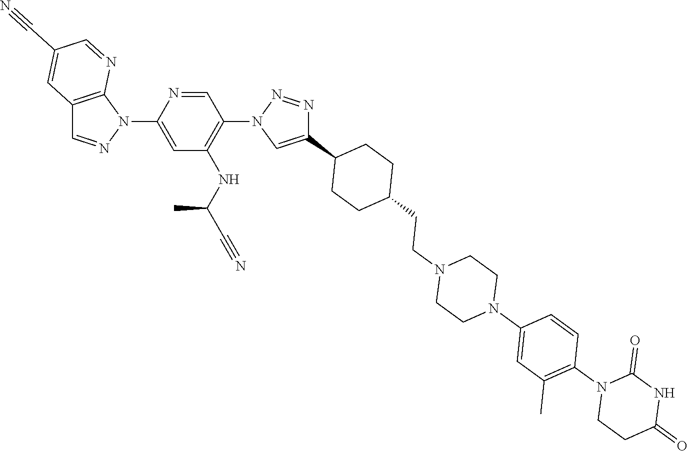 [SMILES: Cc1cc(N2CCN(CC[C@H]3CC[C@H](c4cn(-c5cnc(-n6ncc7cc(C#N)cnc76)cc5N[C@H](C)C#N)nn4)CC3)CC2)ccc1N1CCC(=O)NC1=O] |
| P-364 | 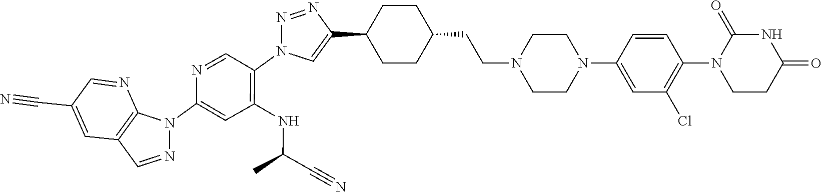 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)c(Cl)c4)CC3)CC2)nn1] |
| P-365 | 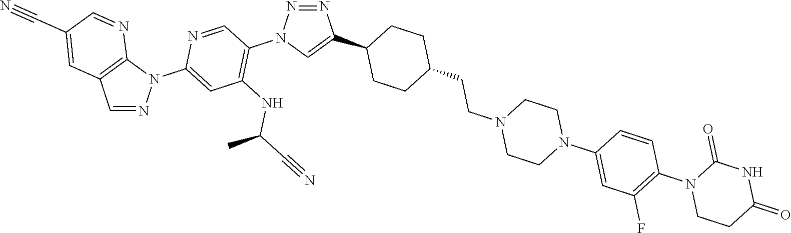 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)c(F)c4)CC3)CC2)nn1] |
| P-366 | 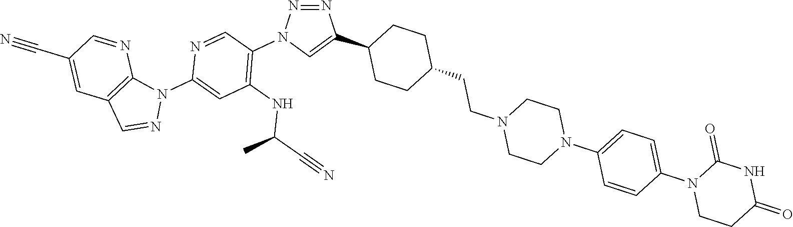 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)cc4)CC3)CC2)nn1] |
| P-367 | 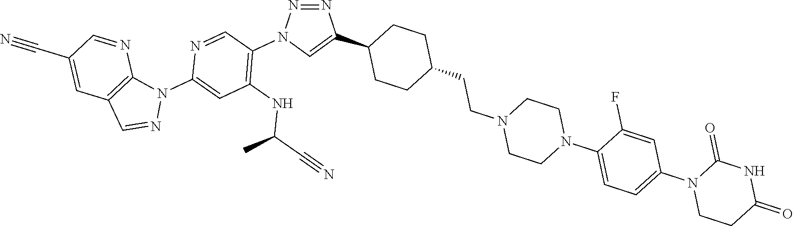 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)cc4F)CC3)CC2)nn1] |
| P-368 | 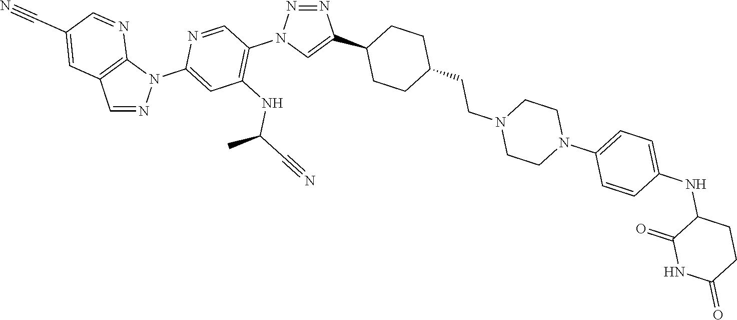 [SMILES: C[C@H](C#N)Nc1cc(-n2ncc3cc(C#N)cnc32)ncc1-n1cc([C@H]2CC[C@H](CCN3CCN(c4ccc(NC5CCC(=O)NC5=O)cc4)CC3)CC2)nn1] |
| P-369 | 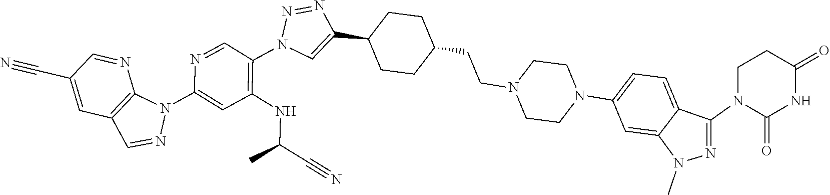 [SMILES: C[C@H](C#N)Nc1cc(-n2ncc3cc(C#N)cnc32)ncc1-n1cc([C@H]2CC[C@H](CCN3CCN(c4ccc5c(N6CCC(=O)NC6=O)nn(C)c5c4)CC3)CC2)nn1] |
| P-370 | 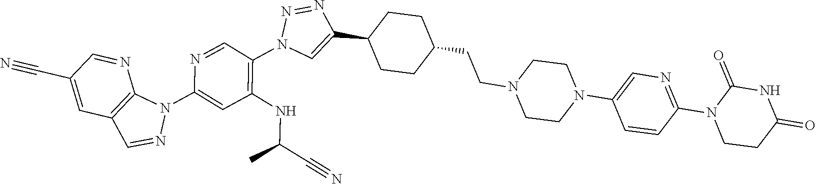 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)nc4)CC3)CC2)nn1] |
| P-371 | 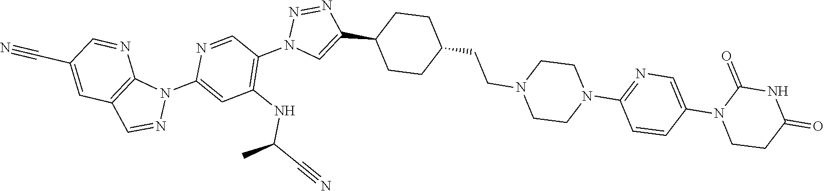 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)cn4)CC3)CC2)nn1] |
| P-372 | 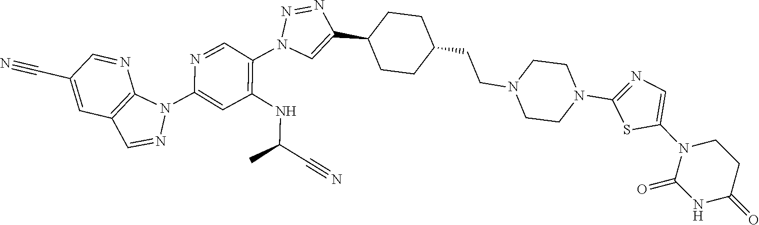 [SMILES: C[C@H](C#N)Nc1cc(-n2ncc3cc(C#N)cnc32)ncc1-n1cc([C@H]2CC[C@H](CCN3CCN(c4ncc(N5CCC(=O)NC5=O)s4)CC3)CC2)nn1] |
| P-373 | 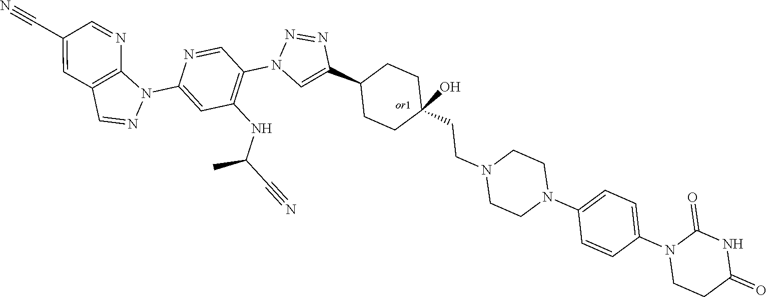 [SMILES: C[C@H](C#N)Nc1cc(-n2ncc3cc(C#N)cnc32)ncc1-n1cc([C@H]2CC[C@](O)(CCN3CCN(c4ccc(N5CCC(=O)NC5=O)cc4)CC3)CC2)nn1] |
| P-375 | 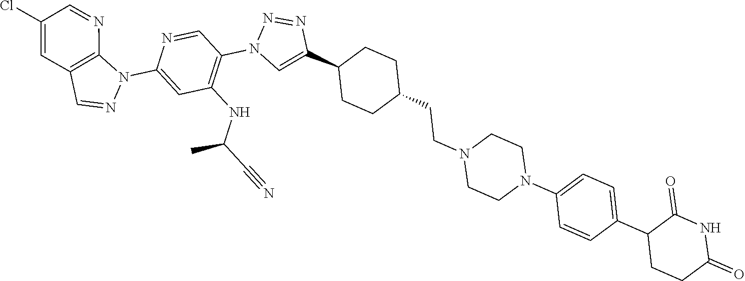 [SMILES: C[C@H](C#N)Nc1cc(-n2ncc3cc(Cl)cnc32)ncc1-n1cc([C@H]2CC[C@H](CCN3CCN(c4ccc(C5CCC(=O)NC5=O)cc4)CC3)CC2)nn1] |
| P-376 | 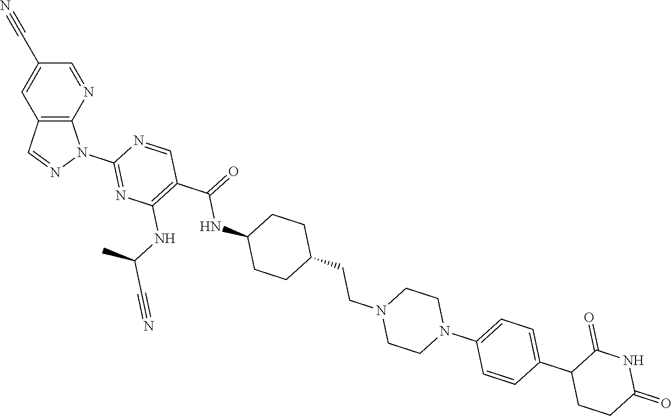 [SMILES: C[C@H](C#N)Nc1nc(-n2ncc3cc(C#N)cnc32)ncc1C(=O)N[C@H]1CC[C@H](CCN2CCN(c3ccc(C4CCC(=O)NC4=O)cc3)CC2)CC1] |
| P-377 | 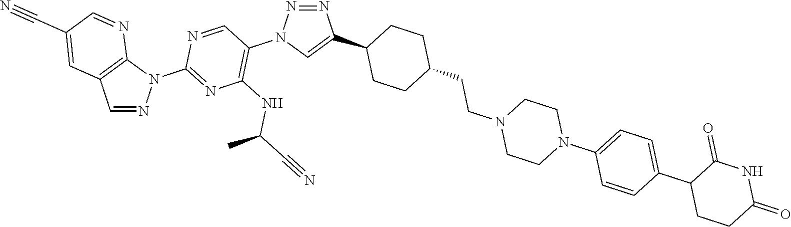 [SMILES: C[C@H](C#N)Nc1nc(-n2ncc3cc(C#N)cnc32)ncc1-n1cc([C@H]2CC[C@H](CCN3CCN(c4ccc(C5CCC(=O)NC5=O)cc4)CC3)CC2)nn1] |
| P-378 | 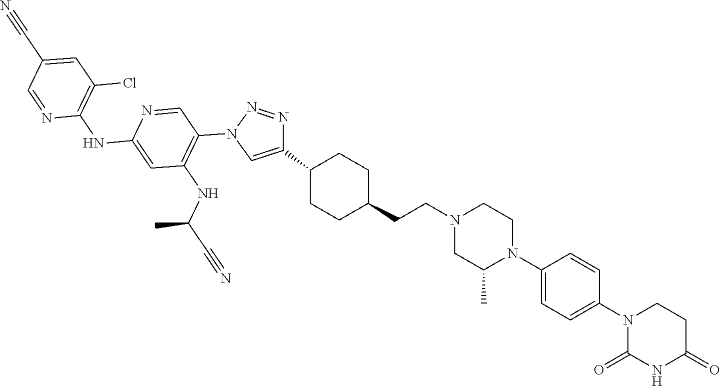 [SMILES: C[C@H](C#N)Nc1cc(Nc2ncc(C#N)cc2Cl)ncc1-n1cc([C@H]2CC[C@H](CCN3CCN(c4ccc(N5CCC(=O)NC5=O)cc4)[C@H](C)C3)CC2)nn1] |
| P-379 | 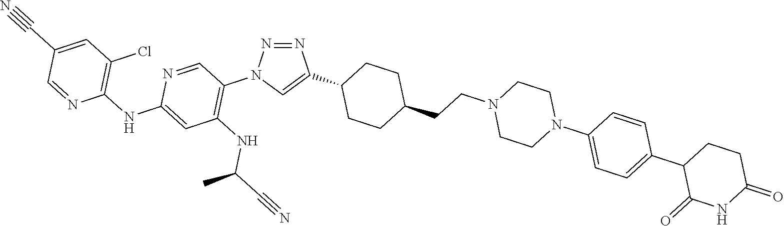 [SMILES: C[C@H](C#N)Nc1cc(Nc2ncc(CN)cc2Cl)ncc1-n1cc([C@H]2CC[C@H](CCN3CCN(c4ccc(C5CCC(=O)NC5=O)cc4)CC3)CC2)nn1] |
| P-380 | 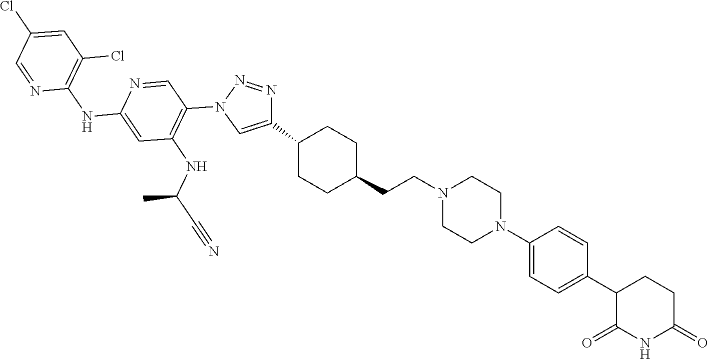 [SMILES: C[C@H](C#N)Nc1cc(Nc2ncc(Cl)cc2Cl)ncc1-n1cc([C@H]2CC[C@H](CCN3CCN(c4ccc(C5CCC(=O)NC5=O)cc4)CC3)CC2)nn1] |
| P-381 | 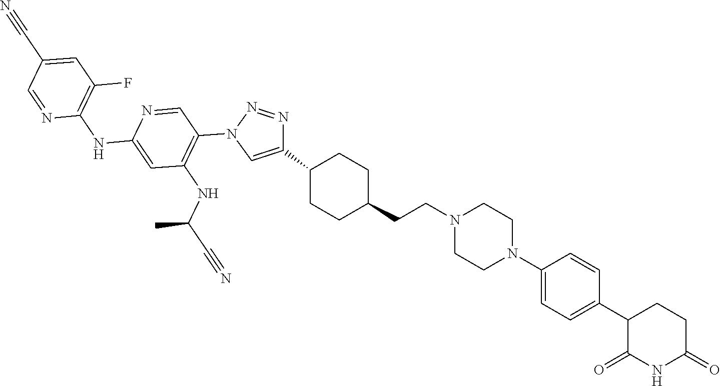 [SMILES: C[C@H](C#N)Nc1cc(Nc2ncc(C#N)cc2F)ncc1-n1cc([C@H]2CC[C@H](CCN3CCN(c4ccc(C5CCC(=O)NC5=O)cc4)CC3)CC2)nn1] |
| P-382 | 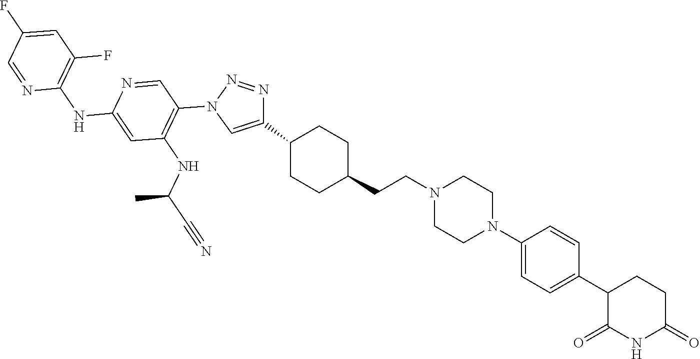 [SMILES: C[C@H](C#N)Nc1cc(Nc2ncc(F)cc2F)ncc1-n1cc([C@H]2CC[C@H](CCN3CCN(c4ccc(C5CCC(=O)NC5=O)cc4)CC3)CC2)nn1] |
| P-383 | 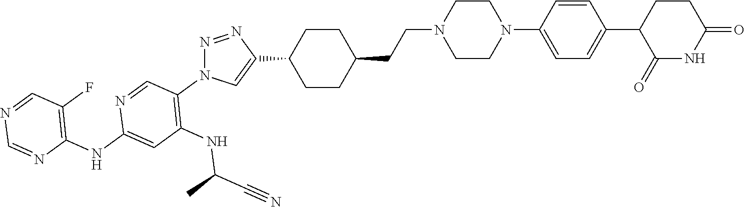 [SMILES: C[C@H](C#N)Nc1cc(Nc2ncncc2F)ncc1-n1cc([C@H]2CC[C@H](CCN3CCN(c4ccc(C5CCC(=O)NC5=O)cc4)CC3)CC2)nn1] |
| P-384 | 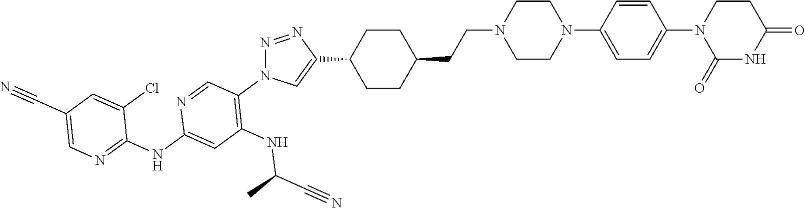 [SMILES: C[C@H](C#N)Nc1cc(Nc2ncc(C#N)cc2Cl)ncc1-n1cc([C@H]2CC[C@H](CCN3CCN(c4ccc(N5CCC(=O)NC5=O)cc4)CC3)CC2)nn1] |
| P-385 | 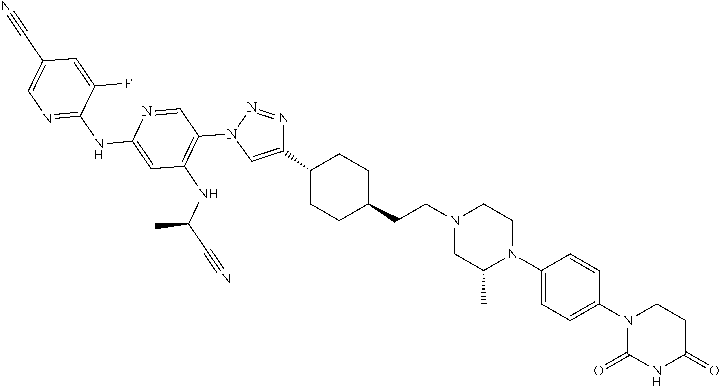 [SMILES: C[C@H](C#N)Nc1cc(Nc2ncc(C#N)cc2F)ncc1-n1cc([C@H]2CC[C@H](CCN3CCN(c4ccc(N5CCC(=O)NC5=O)cc4)[C@H](C)C3)CC2)nn1] |
| P-386 | 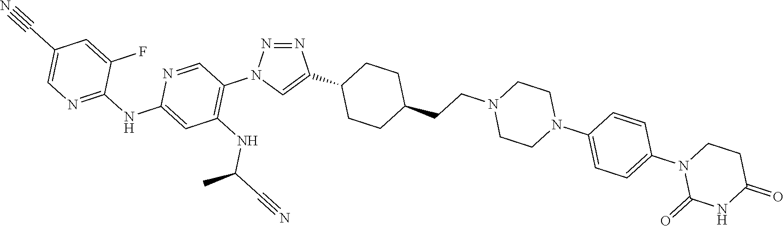 [SMILES: C[C@H](C#N)Nc1cc(Nc2ncc(CN)cc2F)ncc1-n1cc([C@H]2CC[C@H](CCN3CCN(c4ccc(N5CCC(=O)NC5=O)cc4)CC3)CC2)nn1] |
| P-387 | 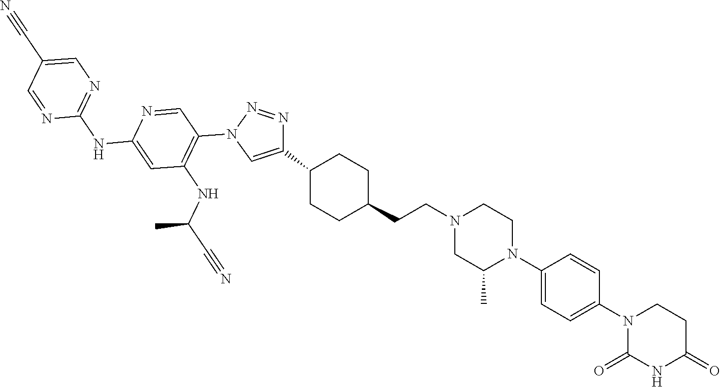 [SMILES: C[C@H](C#N)Nc1cc(Nc2ncc(C#N)cn2)ncc1-n1cc([C@H]2CC[C@H](CCN3CCN(c4ccc(N5CCC(=O)NC5=O)cc4)[C@H](C)C3)CC2)nn1] |
| P-388 | 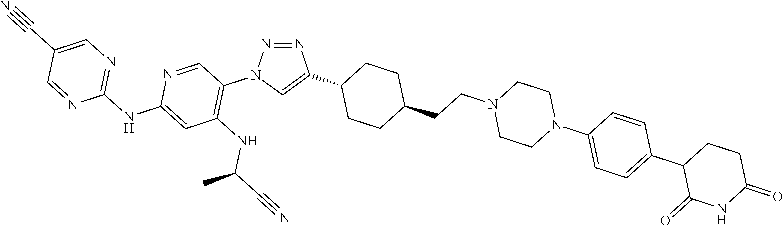 [SMILES: C[C@H](C#N)Nc1cc(Nc2ncc(CN)cn2)ncc1-n1cc([C@H]2CC[C@H](CCN3CCN(c4ccc(C5CCC(=O)NC5=O)cc4)CC3)CC2)nn1] |
| P-389 | 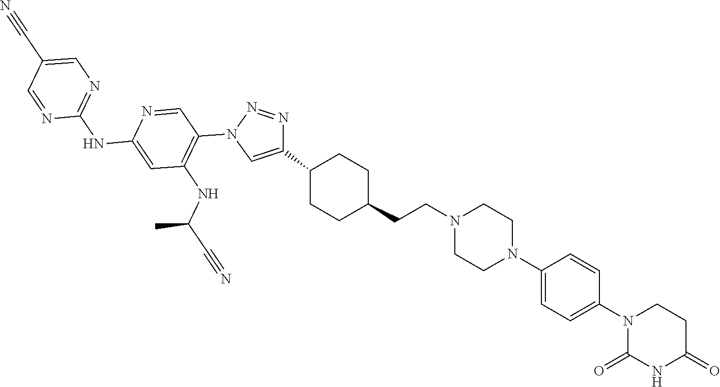 [SMILES: C[C@H](C#N)Nc1cc(Nc2ncc(C#N)cn2)ncc1-n1cc([C@H]2CC[C@H](CCN3CCN(c4ccc(N5CCC(=O)NC5=O)cc4)CC3)CC2)nn1] |
| P-390 | 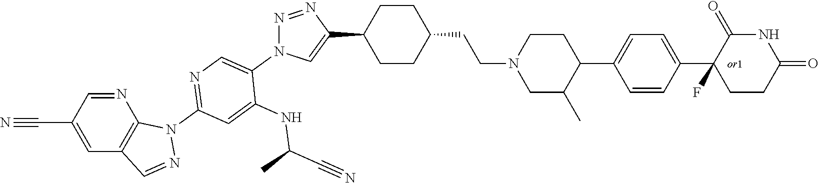 [SMILES: CC1CN(CC[C@H]2CC[C@H](c3cn(-c4cnc(-n5ncc6cc(C#N)cnc65)cc4N[C@H](C)C#N)nn3)CC2)CCC1c1ccc([C@@]2(F)CCC(=O)NC2=O)cc1] |
| P-391 | 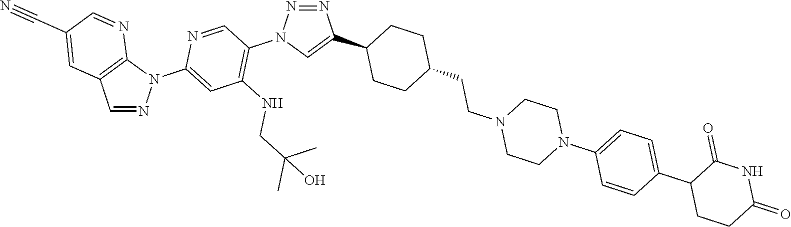 [SMILES: CC(C)(O)CNc1cc(-n2ncc3cc(C#N)cnc32)ncc1-n1cc([C@H]2CC[C@H](CCN3CCN(c4ccc(C5CCC(=O)NC5=O)cc4)CC3)CC2)nn1] |
| P-392 | 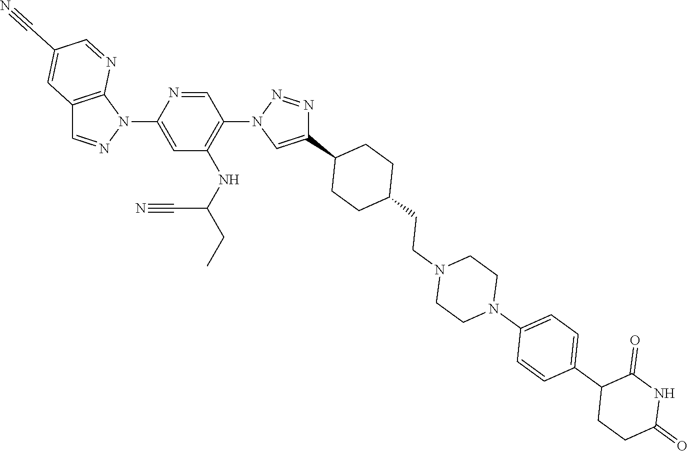 [SMILES: CCC(C#N)Nc1cc(-n2ncc3cc(C#N)cnc32)ncc1-n1cc([C@H]2CC[C@H](CCN3CCN(c4ccc(C5CCC(=O)NC5=O)cc4)CC3)CC2)nn1] |
| P-393 | 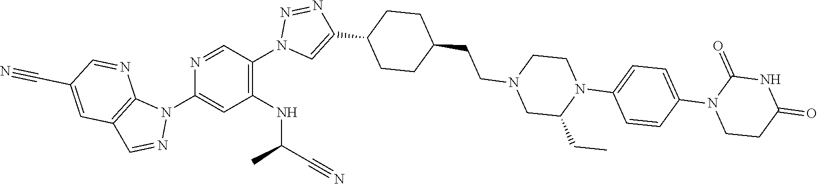 [SMILES: CC[C@@H]1CN(CC[C@H]2CC[C@H](c3cn(-c4cnc(-n5ncc6cc(C#N)cnc65)cc4N[C@H](C)C#N)nn3)CC2)CCN1c1ccc(N2CCC(=O)NC2=O)cc1] |
| P-394 | 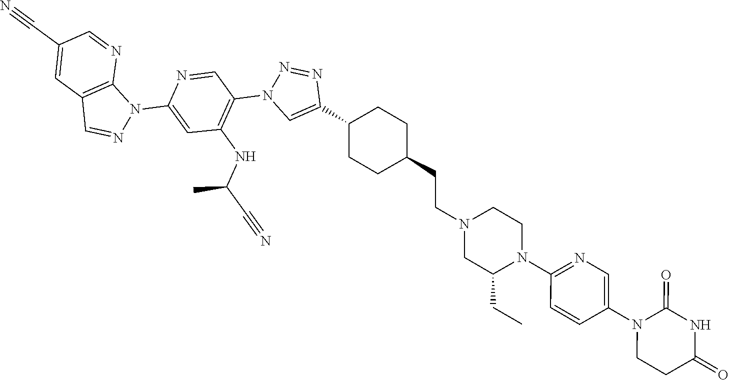 [SMILES: CC[C@@H]1CN(CC[C@H]2CC[C@H](c3cn(-c4cnc(-n5ncc6cc(C#N)cnc65)cc4N[C@H](C)C#N)nn3)CC2)CCN1c1ccc(N2CCC(=O)NC2=O)cn1] |
| P-395 | 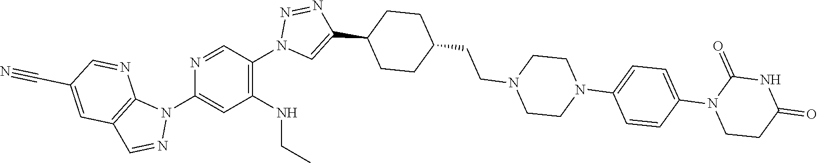 [SMILES: CCNc1cc(-n2ncc3cc(C#N)cnc32)ncc1-n1cc([C@H]2CC[C@H](CCN3CCN(c4ccc(N5CCC(=O)NC5=O)cc4)CC3)CC2)nn1] |
| P-396 | 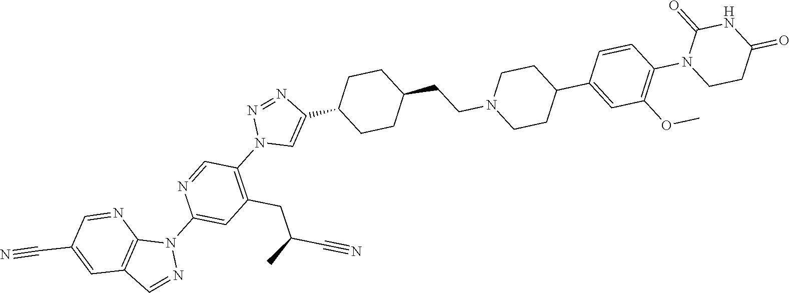 [SMILES: COc1cc(C2CCN(CC[C@H]3CC[C@H](c4cn(-c5cnc(-n6ncc7cc(C#N)cnc76)cc5C[C@H](C)C#N)nn4)CC3)CC2)ccc1N1CCC(=O)NC1=O] |
| P-397 | 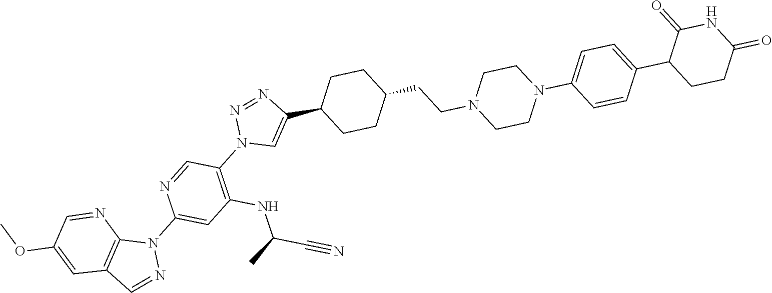 [SMILES: COc1cnc2c(cnn2-c2cc(N[C@H](C)C#N)c(-n3cc([C@H]4CC[C@H](CCN5CCN(c6ccc(C7CCC(=O)NC7=O)cc6)CC5)CC4)nn3)cn2)c1] |
| P-398 | 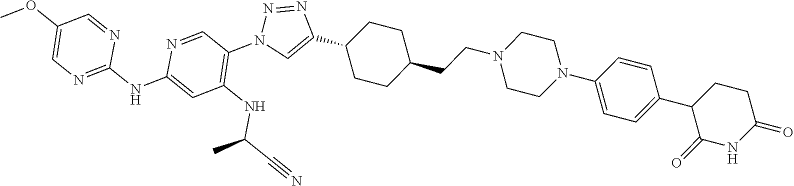 [SMILES: COc1cnc(Nc2cc(N[C@H](C)C#N)c(-n3cc([C@H]4CC[C@H](CCN5CCN(c6ccc(C7CCC(=O)NC7=O)cc6)CC5)CC4)nn3)cn2)nc1] |
| P-399 | 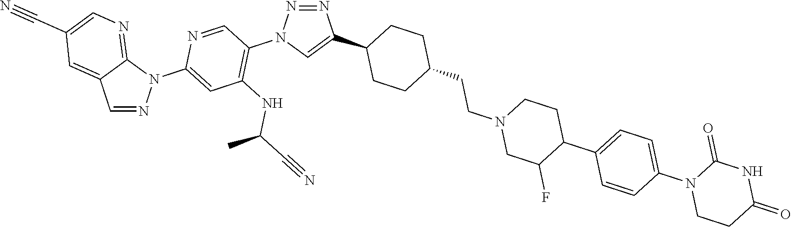 [SMILES: C[C@H](C#N)Nc1cc(-n2ncc3cc(C#N)cnc32)ncc1-n1cc([C@H]2CC[C@H](CCN3CCC(c4ccc(N5CCC(=O)NC5=O)cc4)C(F)C3)CC2)nn1] |
| P-400 | 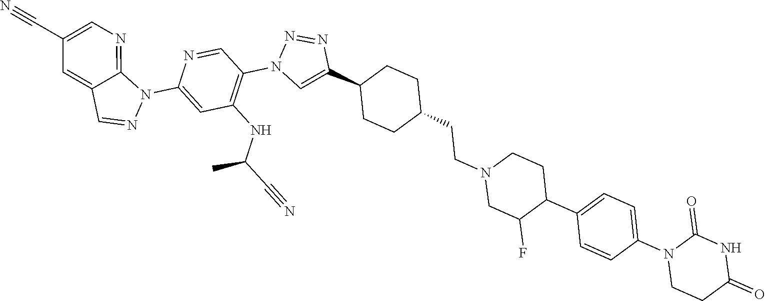 [SMILES: C[C@H](C#N)Nc1cc(-n2ncc3cc(C#N)cnc32)ncc1-n1cc([C@H]2CC[C@H](CCN3CCC(c4ccc(N5CCC(=O)NC5=O)cc4)C(F)C3)CC2)nn1] |
| P-401 | 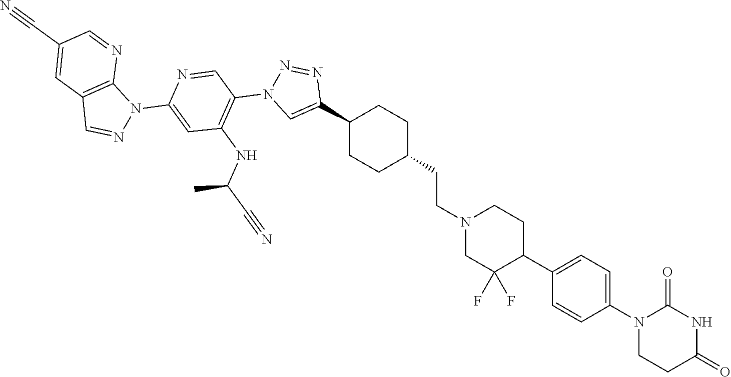 [SMILES: C[C@H](C#N)Nc1cc(-n2ncc3cc(C#N)cnc32)ncc1-n1cc([C@H]2CC[C@H](CCN3CCC(c4ccc(N5CCC(=O)NC5=O)cc4)C(F)(F)C3)CC2)nn1] |
| P-402 | 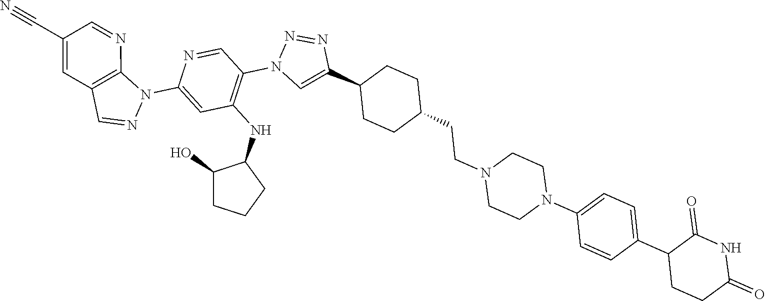 [SMILES: N#Cc1cnc2c(cnn2-c2cc(N[C@H]3CCC[C@H]3O)c(-n3cc([C@H]4CC[C@H](CCN5CCN(c6ccc(C7CCC(=O)NC7=O)cc6)CC5)CC4)nn3)cn2)c1] |
| P-403 | 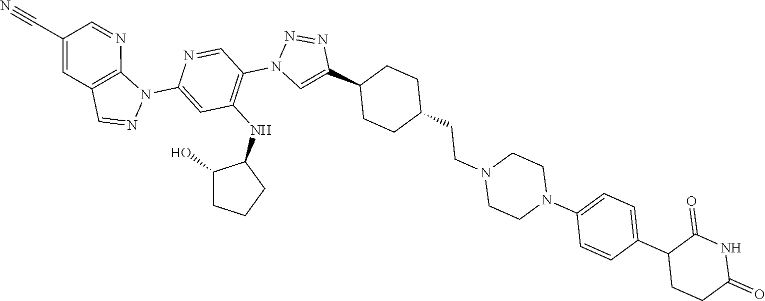 [SMILES: N#Cc1cnc2c(cnn2-c2cc(N[C@H]3CCC[C@@H]3O)c(-n3cc([C@H]4CC[C@H](CCN5CCN(c6ccc(C7CCC(=O)NC7=O)cc6)CC5)CC4)nn3)cn2)c1] |
| P-405 | 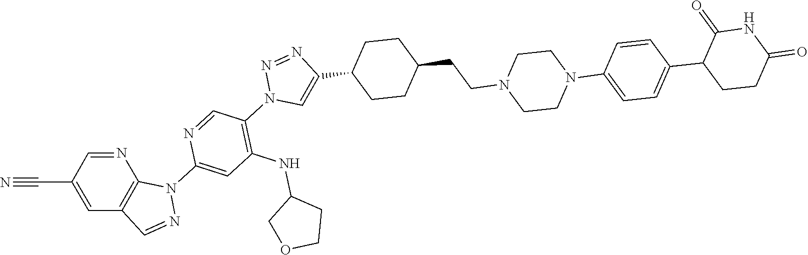 [SMILES: N#Cc1cnc2c(cnn2-c2cc(NC3CCOC3)c(-n3cc([C@H]4CC[C@H](CCN5CCN(c6ccc(C7CCC(=O)NC7=O)cc6)CC5)CC4)nn3)cn2)c1] |
| P-406 | 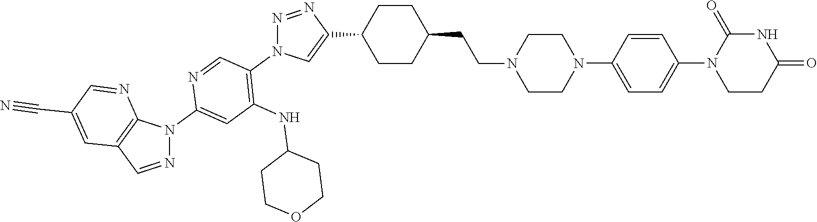 [SMILES: N#Cc1cnc2c(cnn2-c2cc(NC3CCOCC3)c(-n3cc([C@H]4CC[C@H](CCN5CCN(c6ccc(N7CCC(=O)NC7=O)cc6)CC5)CC4)nn3)cn2)c1] |
| P-407 | 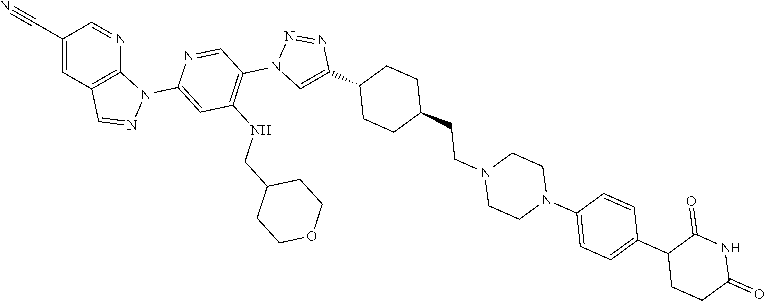 [SMILES: N#Cc1cnc2c(cnn2-c2cc(NCC3CCOCC3)c(-n3cc([C@H]4CC[C@H](CCN5CCN(c6ccc(C7CCC(=O)NC7=O)cc6)CC5)CC4)nn3)cn2)c1] |
| P-408 | 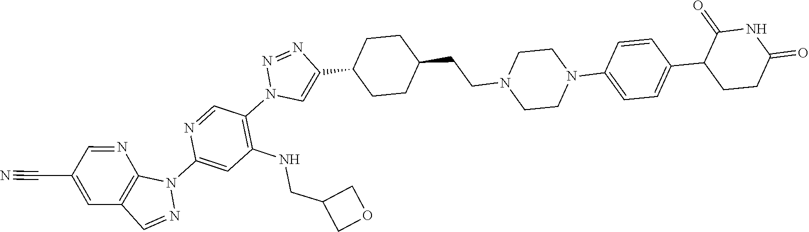 [SMILES: N#Cc1cnc2c(cnn2-c2cc(NCC3COC3)c(-n3cc([C@H]4CC[C@H](CCN5CCN(c6ccc(C7CCC(=O)NC7=O)cc6)CC5)CC4)nn3)cn2)c1] |
| P-409 | 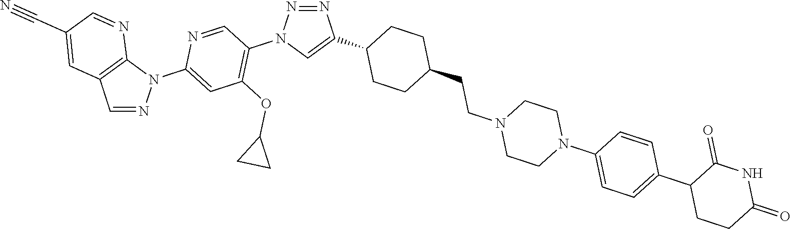 [SMILES: N#Cc1cnc2c(cnn2-c2cc(OC3CC3)c(-n3cc([C@H]4CC[C@H](CCN5CCN(c6ccc(C7CCC(=O)NC7=O)cc6)CC5)CC4)nn3)cn2)c1] |
| P-410 | 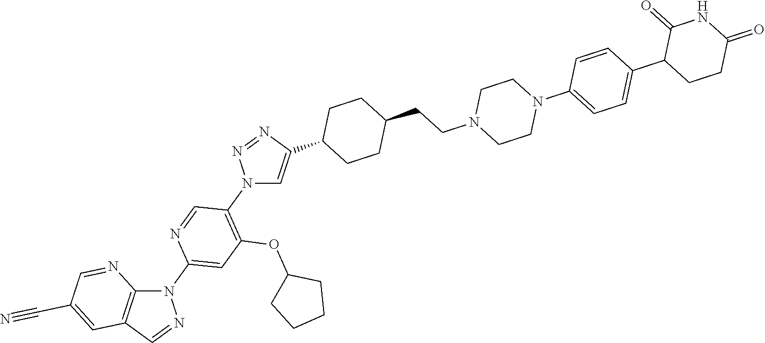 [SMILES: N#Cc1cnc2c(cnn2-c2cc(OC3CCCC3)c(-n3cc([C@H]4CC[C@H](CCN5CCN(c6ccc(C7CCC(=O)NC7=O)cc6)CC5)CC4)nn3)cn2)c1] |
| P-411 | 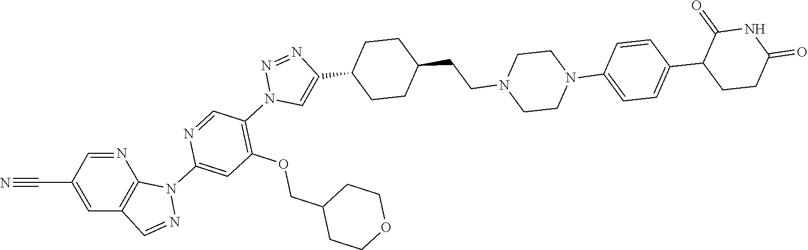 [SMILES: N#Cc1cnc2c(cnn2-c2cc(OCC3CCOCC3)c(-n3cc([C@H]4CC[C@H](CCN5CCN(c6ccc(C7CCC(=O)NC7=O)cc6)CC5)CC4)nn3)cn2)c1] |
| P-412 | 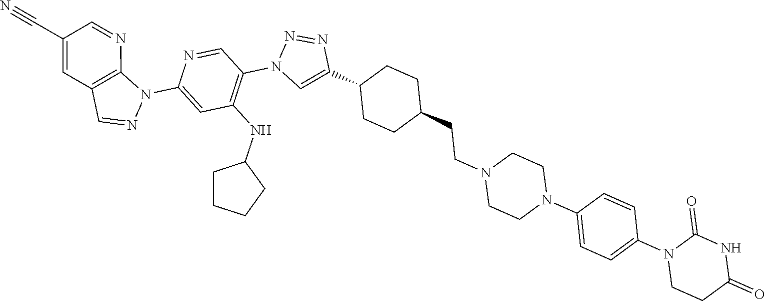 [SMILES: N#Cc1cnc2c(cnn2-c2cc(NC3CCCC3)c(-n3cc([C@H]4CC[C@H](CCN5CCN(c6ccc(N7CCC(=O)NC7=O)cc6)CC5)CC4)nn3)cn2)c1] |
| P-413 | 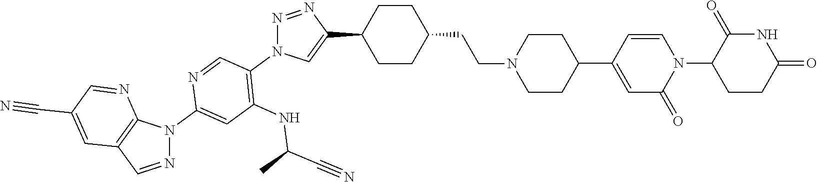 [SMILES: C[C@H](C#N)Nc1cc(-n2ncc3cc(C#N)cnc32)ncc1-n1cc([C@H]2CC[C@H](CCN3CCC(c4ccn(C5CCC(=O)NC5=O)c(=O)c4)CC3)CC2)nn1] |
| P-414 | 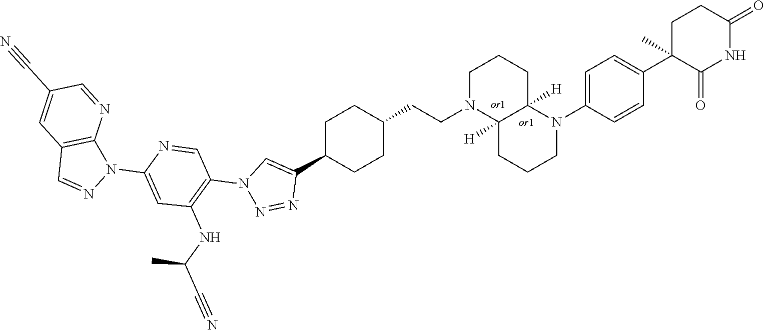 [SMILES: C[C@H](C#N)Nc1cc(-n2ncc3cc(C#N)cnc32)ncc1-n1cc([C@H]2CC[C@H](CCN3CCC[C@@H]4[C@H]3CCCN4c3ccc([C@@]4(C)CCC(=O)NC4=O)cc3)CC2)nn1] |
| P-415 | 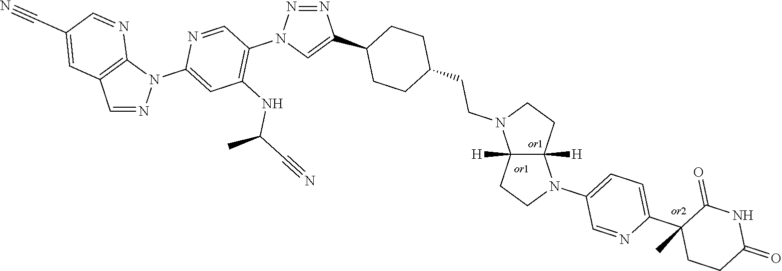 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc([C@@]4(C)CCC(=O)NC4=O)nc3)CC2)nn1] |
| P-416 | 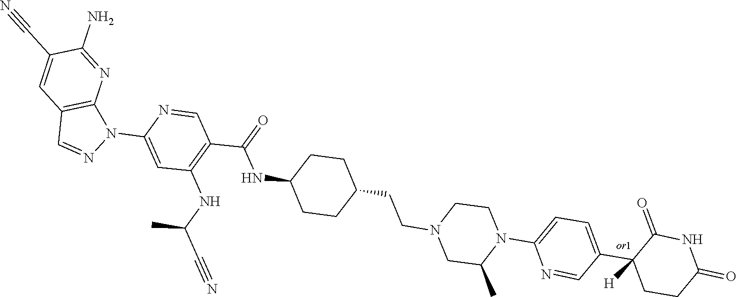 [SMILES: C[C@H](C#N)Nc1cc(-n2ncc3cc(C#N)c(N)nc32)ncc1C(=O)N[C@H]1CC[C@H](CCN2CCN(c3ccc([C@H]4CCC(=O)NC4=O)cn3)[C@@H](C)C2)CC1] |
| P-417 | 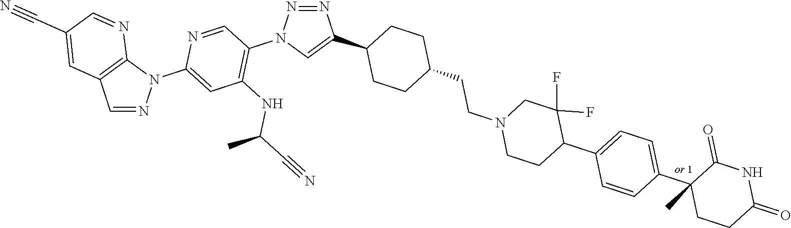 [SMILES: C[C@H](C#N)Nc1cc(-n2ncc3cc(C#N)cnc32)ncc1-n1cc([C@H]2CC[C@H](CCN3CCC(c4ccc([C@@]5(C)CCC(=O)NC5=O)cc4)C(F)(F)C3)CC2)nn1] |
| P-418 | 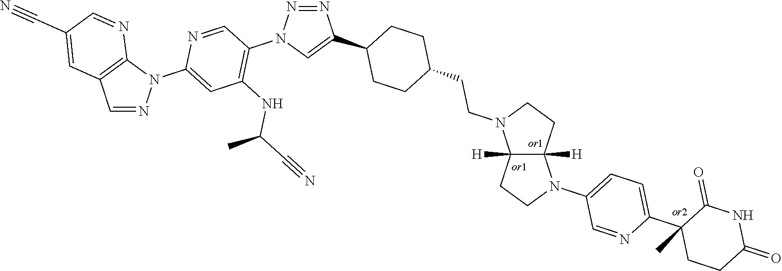 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc([C@@]4(C)CCC(=O)NC4=O)nc3)CC2)nn1] |
| P-419 | 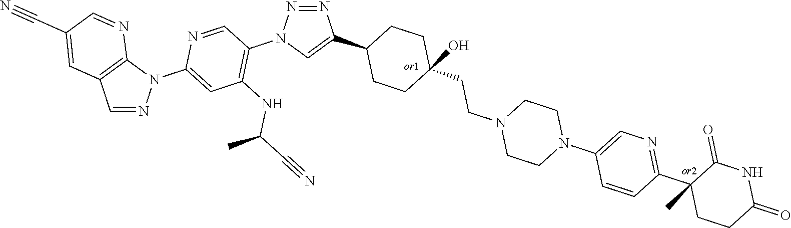 [SMILES: C[C@H](C#N)Nc1cc(-n2ncc3cc(C#N)cnc32)ncc1-n1cc([C@H]2CC[C@](O)(CCN3CCN(c4ccc([C@@]5(C)CCC(=O)NC5=O)nc4)CC3)CC2)nn1] |
| P-420 | 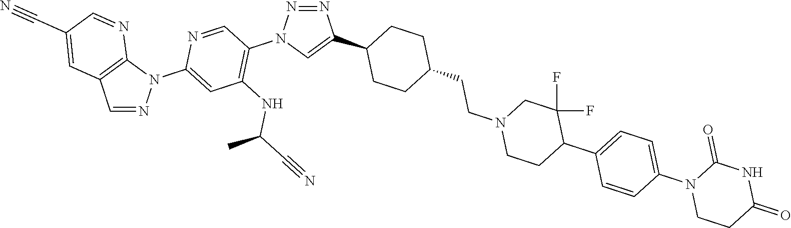 [SMILES: C[C@H](C#N)Nc1cc(-n2ncc3cc(C#N)cnc32)ncc1-n1cc([C@H]2CC[C@H](CCN3CCC(c4ccc(N5CCC(=O)NC5=O)cc4)C(F)(F)C3)CC2)nn1] |
| P-421 | 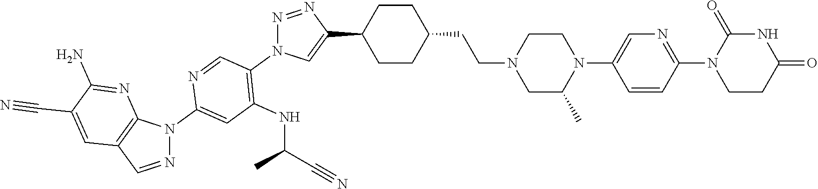 [SMILES: C[C@H](C#N)Nc1cc(-n2ncc3cc(C#N)c(N)nc32)ncc1-n1cc([C@H]2CC[C@H](CCN3CCN(c4ccc(N5CCC(=O)NC5=O)nc4)[C@H](C)C3)CC2)nn1] |
| P-422 | 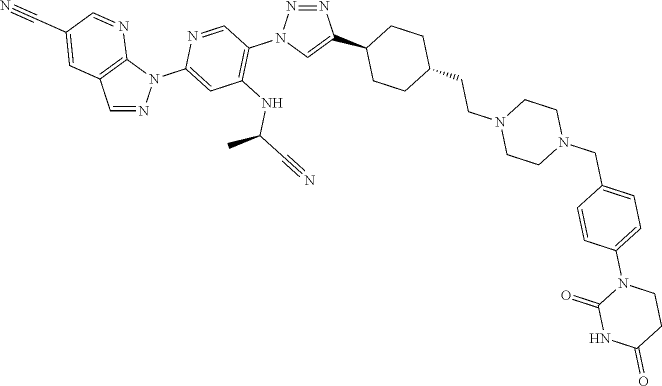 [SMILES: C[C@H](C#N)Nc1cc(-n2ncc3cc(C#N)cnc32)ncc1-n1cc([C@H]2CC[C@H](CCN3CCN(Cc4ccc(N5CCC(=O)NC5=O)cc4)CC3)CC2)nn1] |
| P-423 | 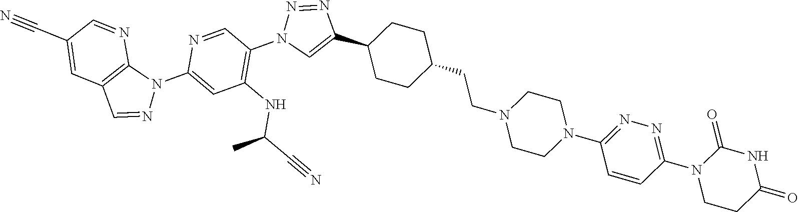 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)nn4)CC3)CC2)nn1] |
| P-424 | 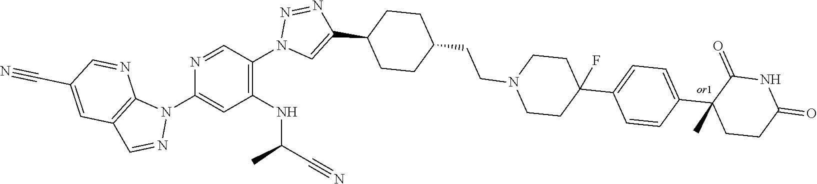 [SMILES: C[C@H](C#N)Nc1cc(-n2ncc3cc(C#N)cnc32)ncc1-n1cc([C@H]2CC[C@H](CCN3CCC(F)(c4ccc([C@@]5(C)CCC(=O)NC5=O)cc4)CC3)CC2)nn1] |
| P-425 | 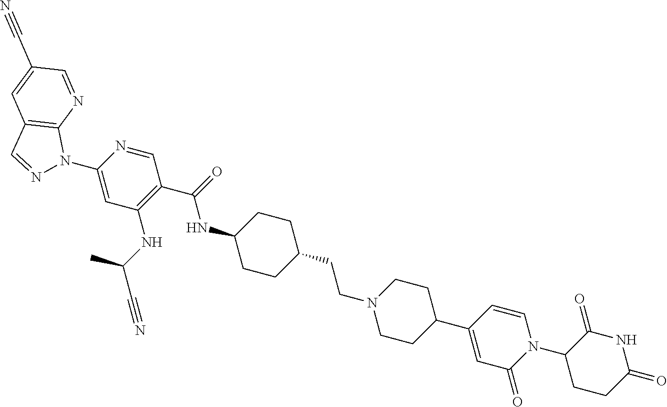 [SMILES: C[C@H](C#N)Nc1cc(-n2ncc3cc(C#N)cnc32)ncc1C(=O)N[C@H]1CC[C@H](CCN2CCC(c3ccn(C4CCC(=O)NC4=O)c(=O)c3)CC2)CC1] |
| P-426 | 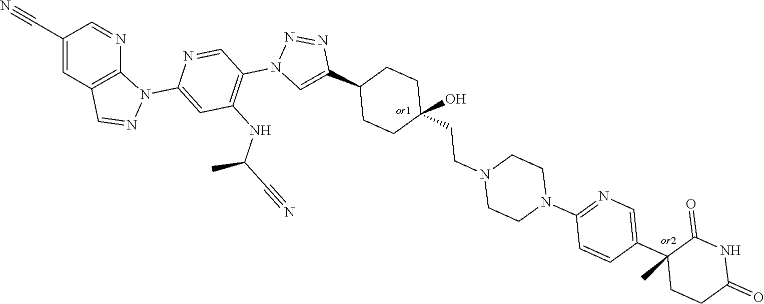 [SMILES: C[C@H](C#N)Nc1cc(-n2ncc3cc(C#N)cnc32)ncc1-n1cc([C@H]2CC[C@](O)(CCN3CCN(c4ccc([C@@]5(C)CCC(=O)NC5=O)cn4)CC3)CC2)nn1] |
| P-427 | 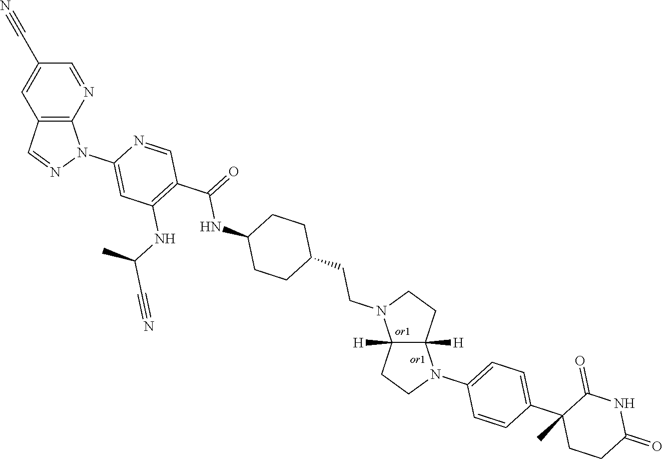 [SMILES: C[C@H](C#N)Nc1cc(-n2ncc3cc(C#N)cnc32)ncc1C(=O)N[C@H]1CC[C@H](CCN2CC[C@H]3[C@@H]2CCN3c2ccc([C@@]3(C)CCC(=O)NC3=O)cc2)CC1] |
| P-428 | 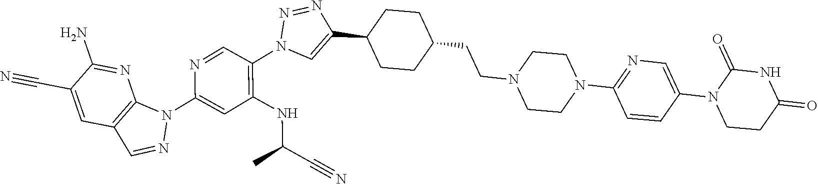 [SMILES: C[C@H](C#N)Nc1cc(-n2ncc3cc(C#N)c(N)nc32)ncc1-n1cc([C@H]2CC[C@H](CCN3CCN(c4ccc(N5CCC(=O)NC5=O)cn4)CC3)CC2)nn1] |
| P-429 | 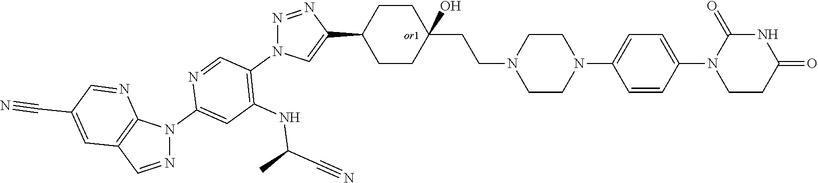 [SMILES: C[C@H](C#N)Nc1cc(-n2ncc3cc(C#N)cnc32)ncc1-n1cc([C@H]2CC[C@](O)(CCN3CCN(c4ccc(N5CCC(=O)NC5=O)cc4)CC3)CC2)nn1] |
| P-430 | 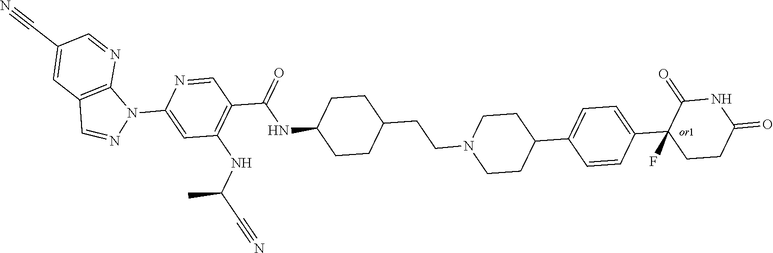 |
| P-432 | 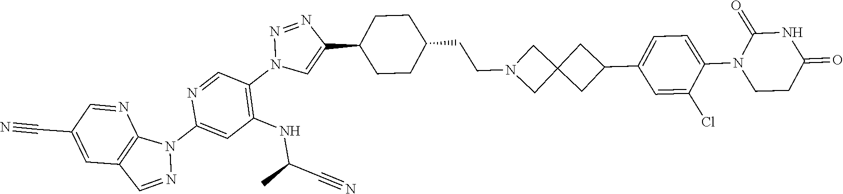 [SMILES: C[C@H](C#N)Nc1cc(-n2ncc3cc(C#N)cnc32)ncc1-n1cc([C@H]2CC[C@H](CCN3CC4(CC(c5ccc(N6CCC(=O)NC6=O)c(Cl)c5)C4)C3)CC2)nn1] |
| P-433 | 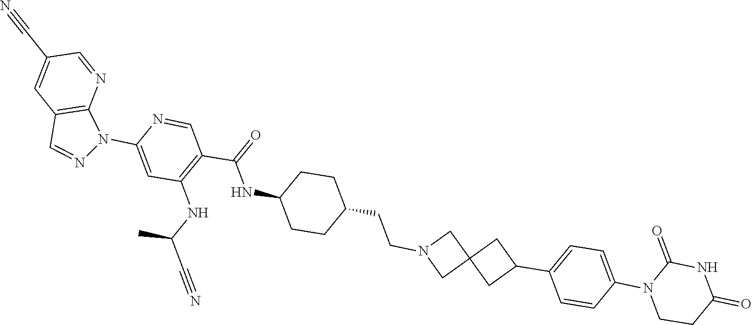 [SMILES: C[C@H](C#N)Nc1cc(-n2ncc3cc(C#N)cnc32)ncc1C(=O)N[C@H]1CC[C@H](CCN2CC3(CC(c4ccc(N5CCC(=O)NC5=O)cc4)C3)C2)CC1] |
| P-434 | 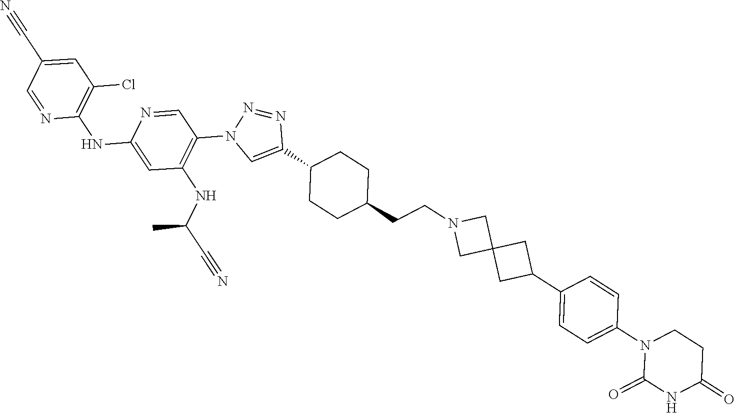 [SMILES: C[C@H](C#N)Nc1cc(Nc2ncc(C#N)cc2Cl)ncc1-n1cc([C@H]2CC[C@H](CCN3CC4(CC(c5ccc(N6CCC(=O)NC6=O)cc5)C4)C3)CC2)nn1] |
| P-435 | 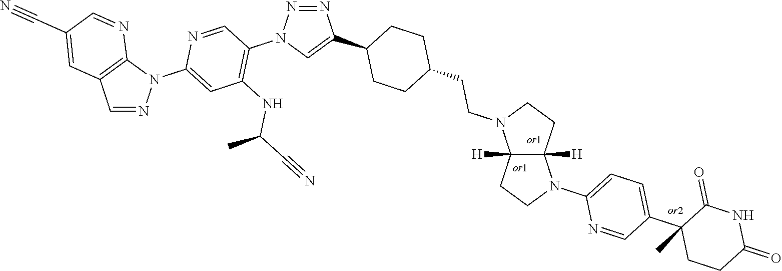 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc([C@@]4(C)CCC(=O)NC4=O)cn3)CC2)nn1] |
| P-436 | 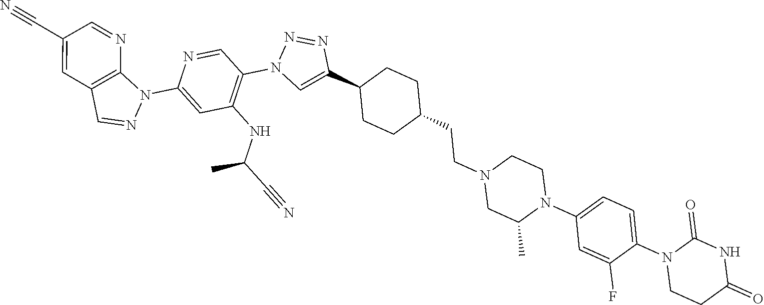 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)c(F)c4)[C@H](C)C3)CC2)nn1] |
| P-437 | 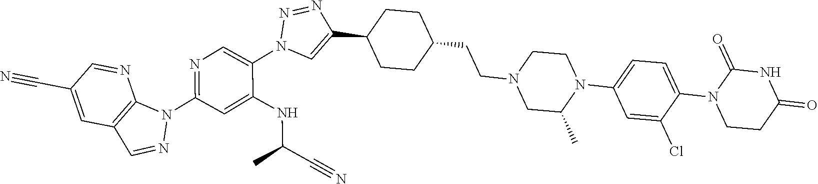 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)c(Cl)c4)[C@H](C)C3)CC2)nn1] |
| P-438 | 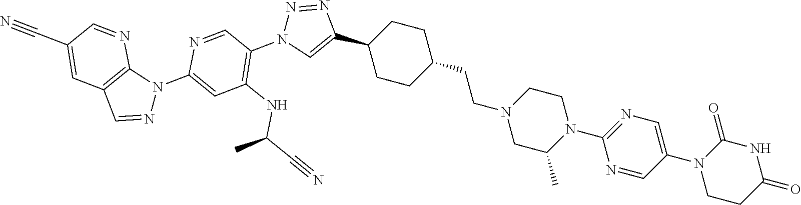 [SMILES: C[C@H](C#N)Nc1cc(-n2ncc3cc(C#N)cnc32)ncc1-n1cc([C@H]2CC[C@H](CCN3CCN(c4ncc(N5CCC(=O)NC5=O)cn4)[C@H](C)C3)CC2)nn1] |
| P-439 | 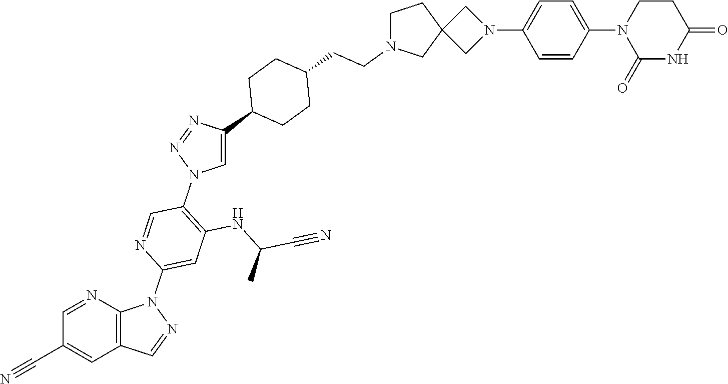 [SMILES: C[C@H](C#N)Nc1cc(-n2ncc3cc(C#N)cnc32)ncc1-n1cc([C@H]2CC[C@H](CCN3CCC4(C3)CN(c3ccc(N5CCC(=O)NC5=O)cc3)C4)CC2)nn1] |
| P-440 | 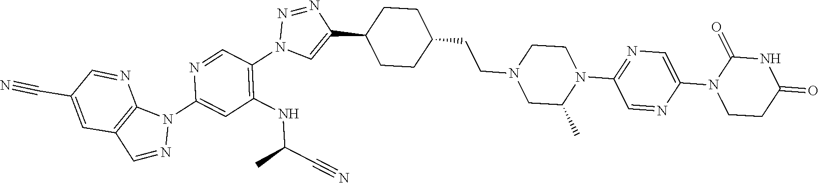 [SMILES: C[C@H](C#N)Nc1cc(-n2ncc3cc(C#N)cnc32)ncc1-n1cc([C@H]2CC[C@H](CCN3CCN(c4cnc(N5CCC(=O)NC5=O)cn4)[C@H](C)C3)CC2)nn1] |
| P-441 | 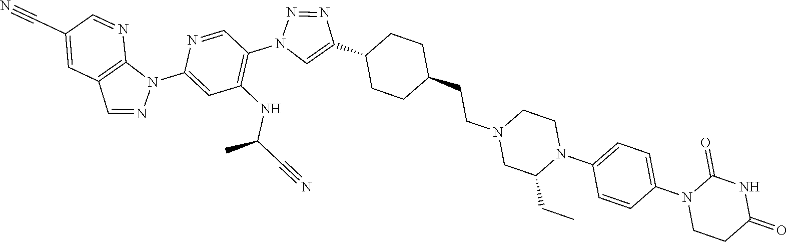 [SMILES: CC[C@@H]1CN(CC[C@H]2CC[C@H](c3cn(-c4cnc(-n5ncc6cc(C#N)cnc65)cc4N[C@H](C)C#N)nn3)CC2)CCN1c1ccc(N2CCC(=O)NC2=O)cc1] |
| P-442 | 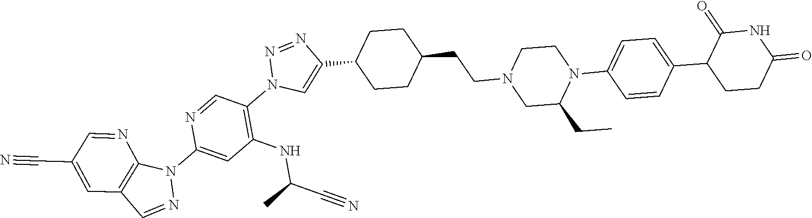 [SMILES: CC[C@H]1CN(CC[C@H]2CC[C@H](c3cn(-c4cnc(-n5ncc6cc(C#N)cnc65)cc4N[C@H](C)C#N)nn3)CC2)CCN1c1ccc(C2CCC(=O)NC2=O)cc1] |
| P-443 | 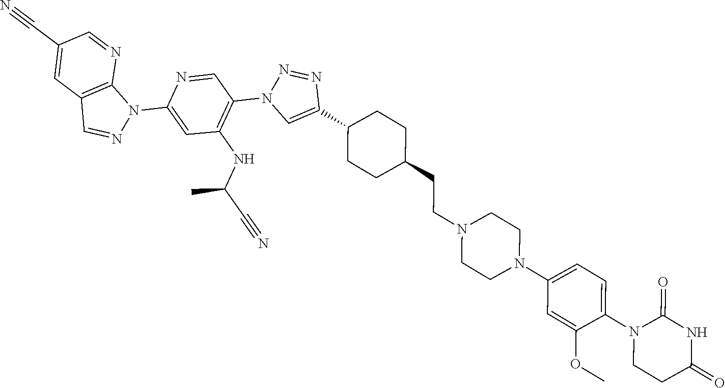 [SMILES: COc1cc(N2CCN(CC[C@H]3CC[C@H](c4cn(-c5cnc(-n6ncc7cc(C#N)cnc76)cc5N[C@H](C)C#N)nn4)CC3)CC2)ccc1N1CCC(=O)NC1=O] |
| P-444 | 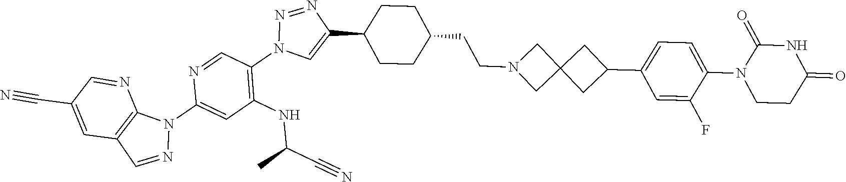 [SMILES: C[C@H](C#N)Nc1cc(-n2ncc3cc(C#N)cnc32)ncc1-n1cc([C@H]2CC[C@H](CCN3CC4(CC(c5ccc(N6CCC(=O)NC6=O)c(F)c5)C4)C3)CC2)nn1] |
| P-445 | 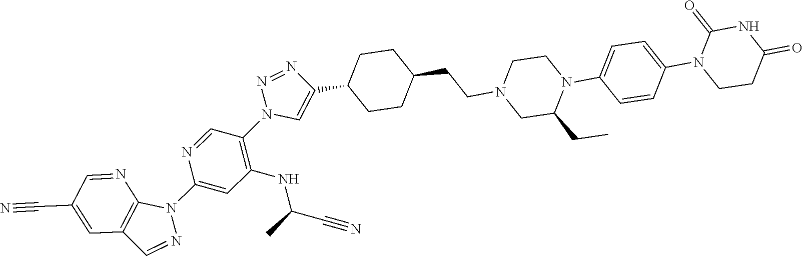 [SMILES: CC[C@H]1CN(CC[C@H]2CC[C@H](c3cn(-c4cnc(-n5ncc6cc(C#N)cnc65)cc4N[C@H](C)C#N)nn3)CC2)CCN1c1ccc(N2CCC(=O)NC2=O)cc1] |
| P-446 | 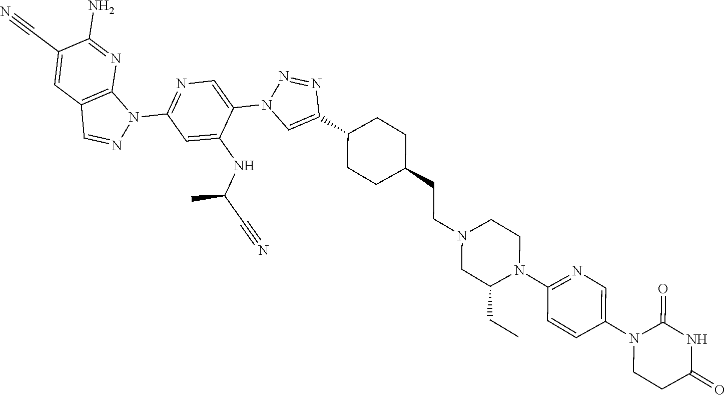 [SMILES: CC[C@@H]1CN(CC[C@H]2CC[C@H](c3cn(-c4cnc(-n5ncc6cc(C#N)c(N)nc65)cc4N[C@H](C)C#N)nn3)CC2)CCN1c1ccc(N2CCC(=O)NC2=O)cn1] |
| P-447 | 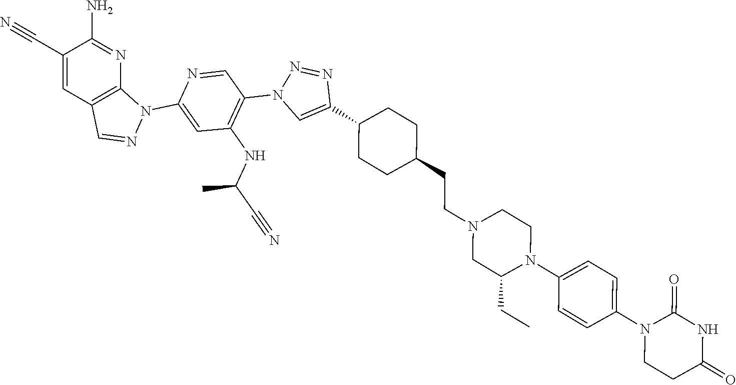 [SMILES: CC[C@@H]1CN(CC[C@H]2CC[C@H](c3cn(-c4cnc(-n5ncc6cc(C#N)c(N)nc65)cc4N[C@H](C)C#N)nn3)CC2)CCN1c1ccc(N2CCC(=O)NC2=O)cc1] |
| P-448 | 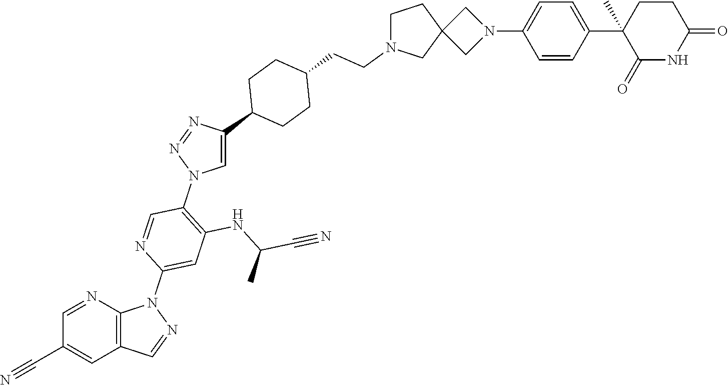 [SMILES: C[C@H](C#N)Nc1cc(-n2ncc3cc(C#N)cnc32)ncc1-n1cc([C@H]2CC[C@H](CCN3CCC4(C3)CN(c3ccc([C@@]5(C)CCC(=O)NC5=O)cc3)C4)CC2)nn1] |
| P-449 | 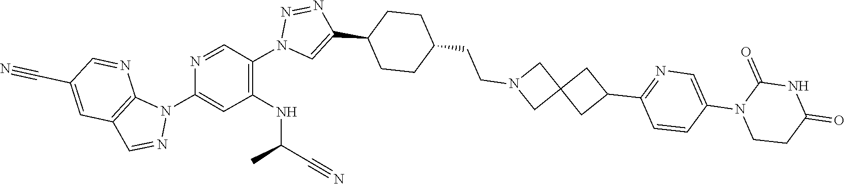 [SMILES: C[C@H](C#N)Nc1cc(-n2ncc3cc(C#N)cnc32)ncc1-n1cc([C@H]2CC[C@H](CCN3CC4(CC(c5ccc(N6CCC(=O)NC6=O)cn5)C4)C3)CC2)nn1] |
| P-452 | 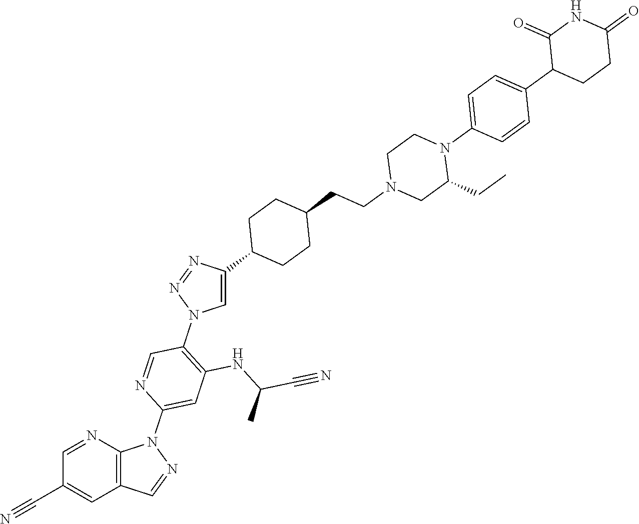 [SMILES: CC[C@@H]1CN(CC[C@H]2CC[C@H](c3cn(-c4cnc(-n5ncc6cc(C#N)cnc65)cc4N[C@H](C)C#N)nn3)CC2)CCN1c1ccc(C2CCC(=O)NC2=O)cc1] |
| P-453 | 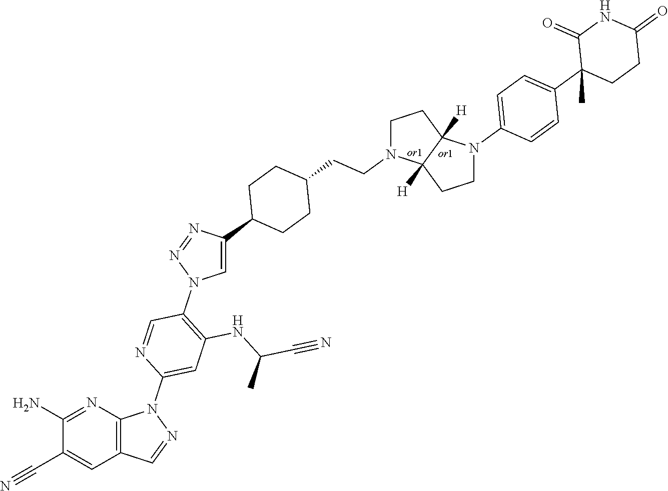 [SMILES: C[C@H](C#N)Nc1cc(-n2ncc3cc(C#N)c(N)nc32)ncc1-n1cc([C@H]2CC[C@H](CCN3CC[C@H]4[C@@H]3CCN4c3ccc([C@@]4(C)CCC(=O)NC4=O)cc3)CC2)nn1] |
| P-454 | 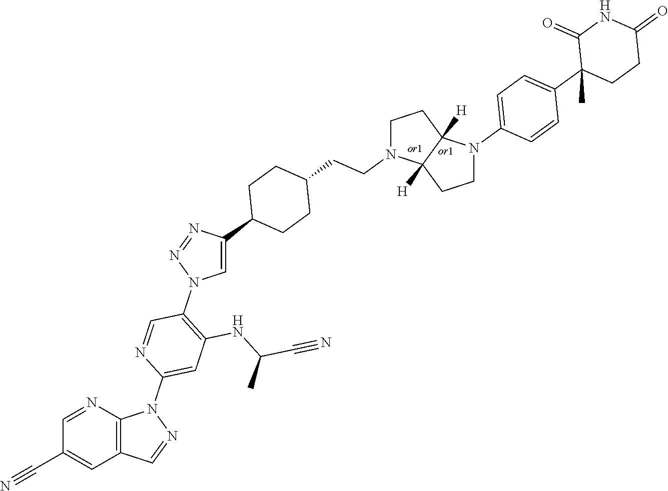 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc([C@@]4(C)CCC(=O)NC4=O)cc3)CC2)nn1] |
| P-455 | 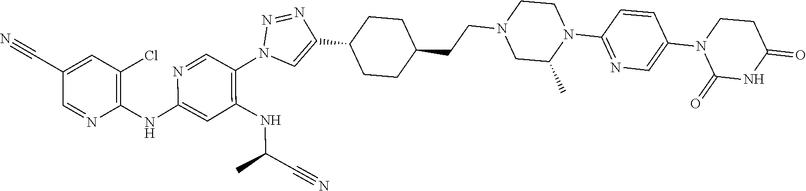 [SMILES: C[C@H](C#N)Nc1cc(Nc2ncc(C#N)cc2Cl)ncc1-n1cc([C@H]2CC[C@H](CCN3CCN(c4ccc(N5CCC(=O)NC5=O)cn4)[C@H](C)C3)CC2)nn1] |
| P-458 | 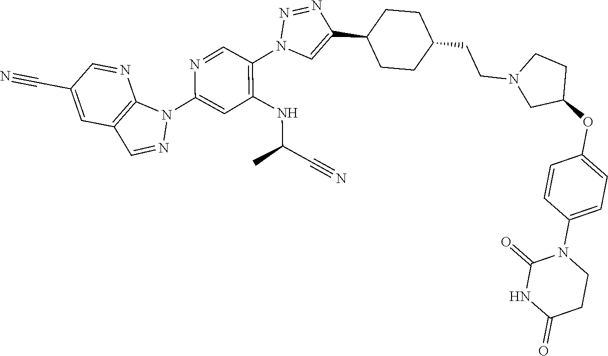 [SMILES: C[C@H](C#N)Nc1cc(-n2ncc3cc(C#N)cnc32)ncc1-n1cc([C@H]2CC[C@H](CCN3CC[C@@H](Oc4ccc(N5CCC(=O)NC5=O)cc4)C3)CC2)nn1] |
| P-459 | 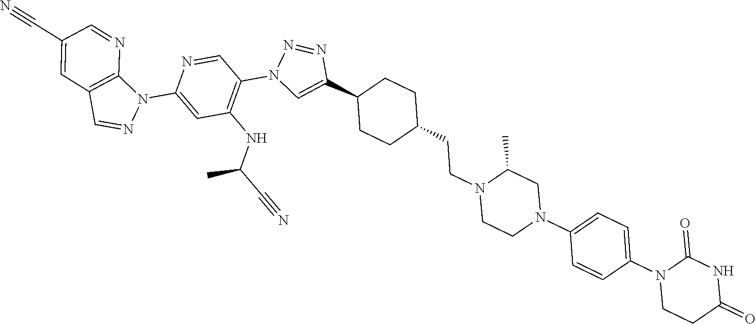 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)cc4)C[C@H]3C)CC2)nn1] |
| P-461 | 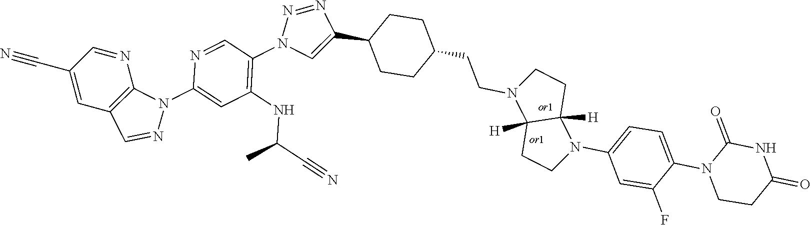 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc(N4CCC(=O)NC4=O)c(F)c3)CC2)nn1] |
| P-462 | 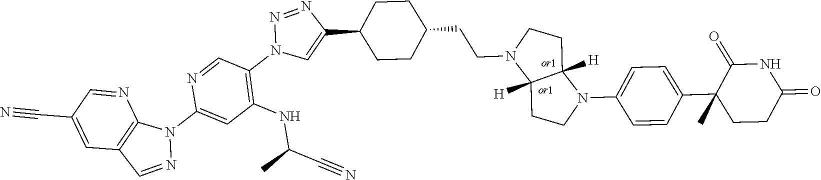 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc([C@@]4(C)CCC(=O)NC4=O)cc3)CC2)nn1] |
| P-463 | 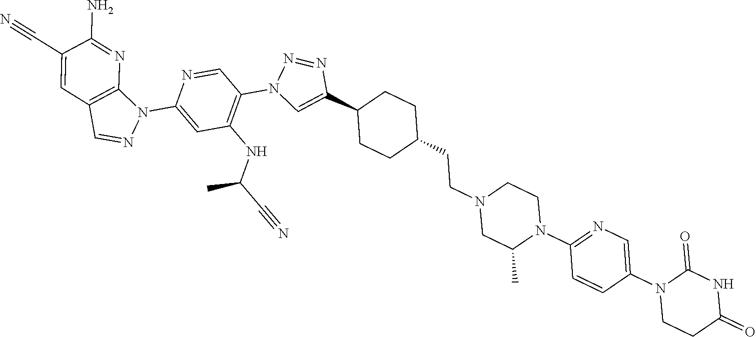 [SMILES: C[C@H](C#N)Nc1cc(-n2ncc3cc(C#N)c(N)nc32)ncc1-n1cc([C@H]2CC[C@H](CCN3CCN(c4ccc(N5CCC(=O)NC5=O)cn4)[C@H](C)C3)CC2)nn1] |
| P-464 | 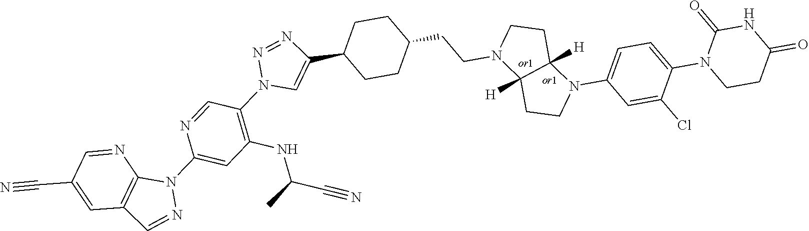 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc(N4CCC(=O)NC4=O)c(Cl)c3)CC2)nn1] |
| P-465 | 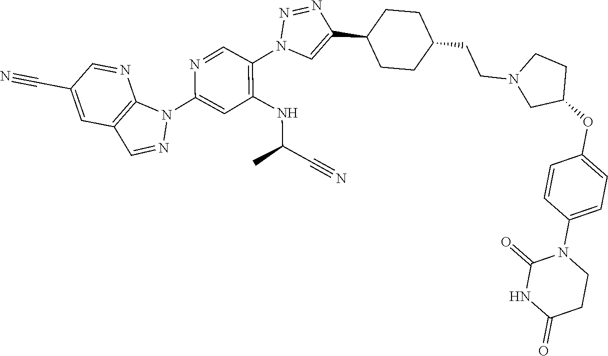 [SMILES: C[C@H](C#N)Nc1cc(-n2ncc3cc(C#N)cnc32)ncc1-n1cc([C@H]2CC[C@H](CCN3CC[C@H](Oc4ccc(N5CCC(=O)NC5=O)cc4)C3)CC2)nn1] |
| P-468 | 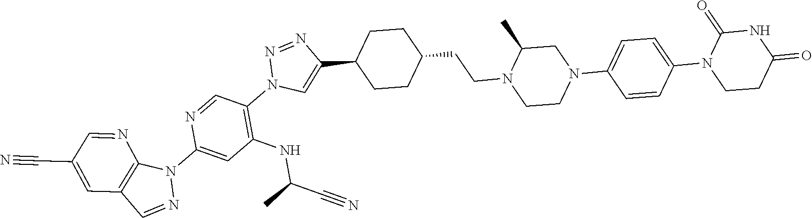 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)cc4)C[C@@H]3C)CC2)nn1] |
| P-470 | 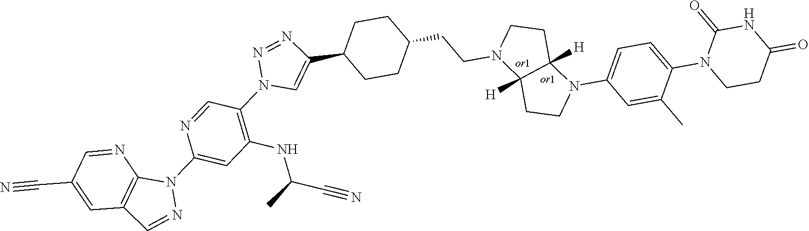 [SMILES: Cc1cc(N2CC[C@H]3[C@@H]2CCN3CC[C@H]2CC[C@H](c3cn(-c4cnc(-n5ncc6cc(C#N)cnc65)cc4N[C@H](C)C#N)nn3)CC2)ccc1N1CCC(=O)NC1=O] |
| P-471 | 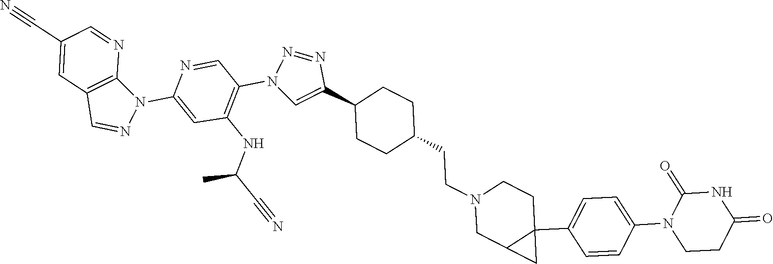 [SMILES: C[C@H](C#N)Nc1cc(-n2ncc3cc(C#N)cnc32)ncc1-n1cc([C@H]2CC[C@H](CCN3CCC4(c5ccc(N6CCC(=O)NC6=O)cc5)CC4C3)CC2)nn1] |
| P-473 | 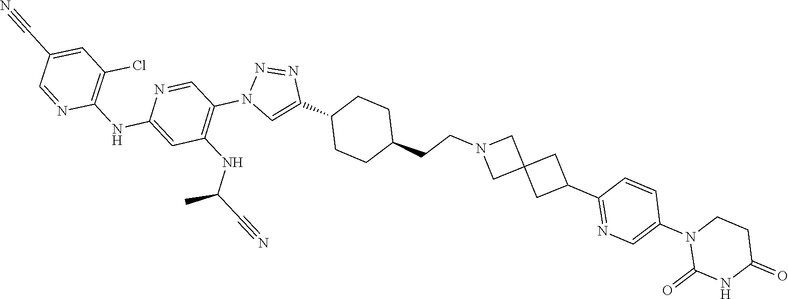 [SMILES: C[C@H](C#N)Nc1cc(Nc2ncc(C#N)cc2Cl)ncc1-n1cc([C@H]2CC[C@H](CCN3CC4(CC(c5ccc(N6CCC(=O)NC6=O)cn5)C4)C3)CC2)nn1] |
| P-474 | 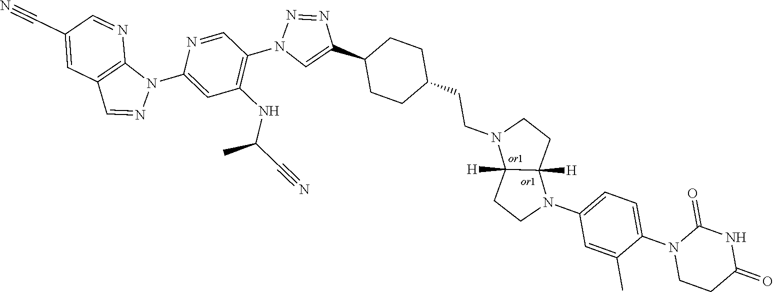 [SMILES: Cc1cc(N2CC[C@H]3[C@@H]2CCN3CC[C@H]2CC[C@H](c3cn(-c4cnc(-n5ncc6cc(C#N)cnc65)cc4N[C@H](C)C#N)nn3)CC2)ccc1N1CCC(=O)NC1=O] |
| P-475 | 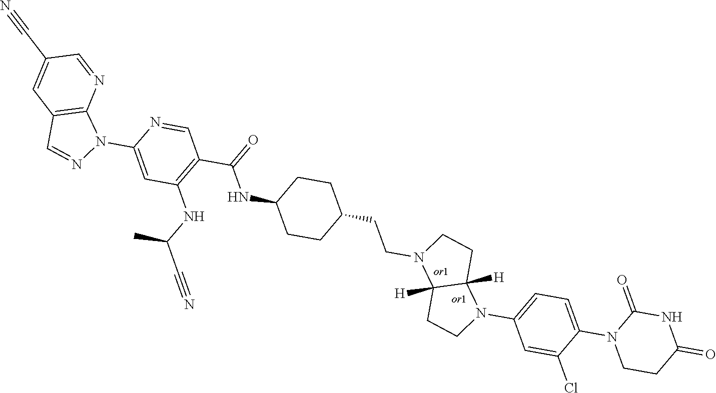 [SMILES: C[C@H](C#N)Nc1cc(-n2ncc3cc(C#N)cnc32)ncc1C(=O)N[C@H]1CC[C@H](CCN2CC[C@H]3[C@@H]2CCN3c2ccc(N3CCC(=O)NC3=O)c(Cl)c2)CC1] |
| P-476 | 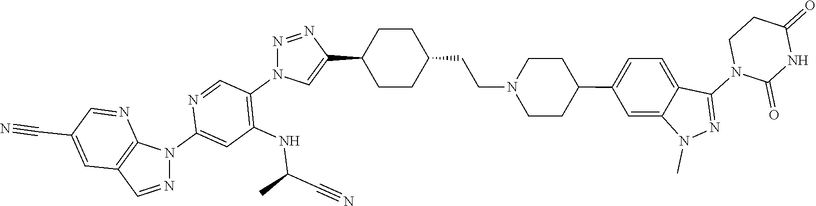 [SMILES: C[C@H](C#N)Nc1cc(-n2ncc3cc(C#N)cnc32)ncc1-n1cc([C@H]2CC[C@H](CCN3CCC(c4ccc5c(N6CCC(=O)NC6=O)nn(C)c5c4)CC3)CC2)nn1] |
| P-477 | 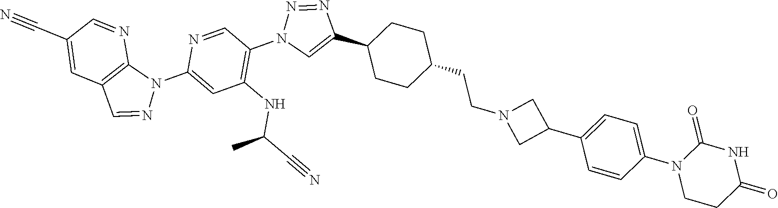 [SMILES: C[C@H](C#N)Nc1cc(-n2ncc3cc(C#N)cnc32)ncc1-n1cc([C@H]2CC[C@H](CCN3CC(c4ccc(N5CCC(=O)NC5=O)cc4)C3)CC2)nn1] |
| P-478 | 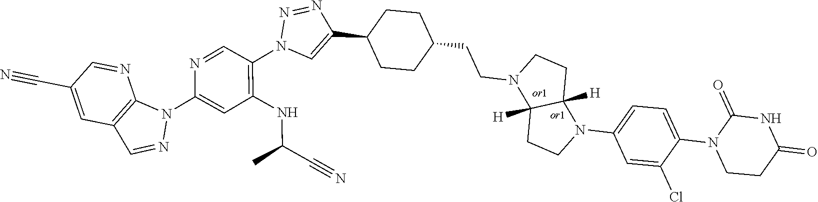 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc(N4CCC(=O)NC4=O)c(Cl)c3)CC2)nn1] |
| P-480 | 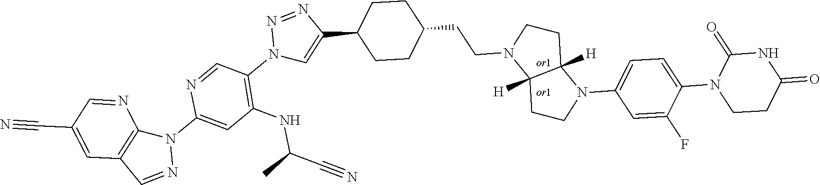 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc(N4CCC(=O)NC4=O)c(F)c3)CC2)nn1] |
| P-481 | 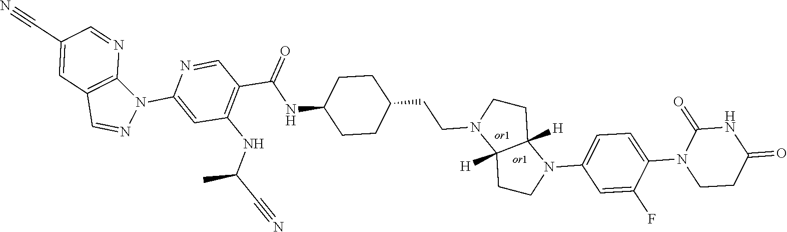 [SMILES: C[C@H](C#N)Nc1cc(-n2ncc3cc(C#N)cnc32)ncc1C(=O)N[C@H]1CC[C@H](CCN2CC[C@H]3[C@@H]2CCN3c2ccc(N3CCC(=O)NC3=O)c(F)c2)CC1] |
| P-483 | 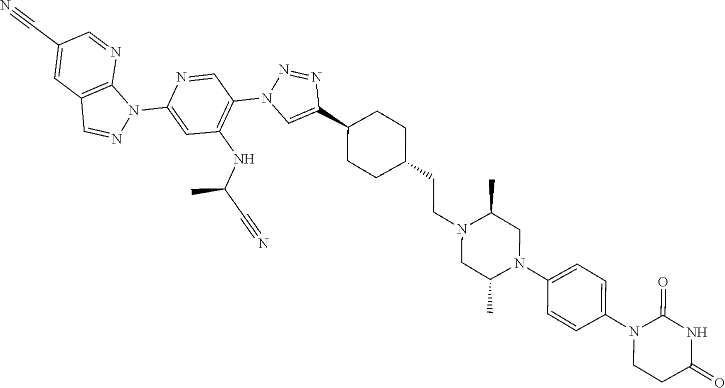 [SMILES: C[C@H](C#N)Nc1cc(-n2ncc3cc(C#N)cnc32)ncc1-n1cc([C@H]2CC[C@H](CCN3C[C@@H](C)N(c4ccc(N5CCC(=O)NC5=O)cc4)C[C@@H]3C)CC2)nn1] |
| P-484 | 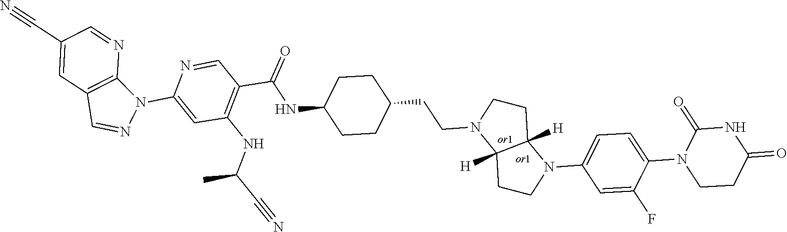 [SMILES: C[C@H](C#N)Nc1cc(-n2ncc3cc(C#N)cnc32)ncc1C(=O)N[C@H]1CC[C@H](CCN2CC[C@H]3[C@@H]2CCN3c2ccc(N3CCC(=O)NC3=O)c(F)c2)CC1] |
| P-486 | 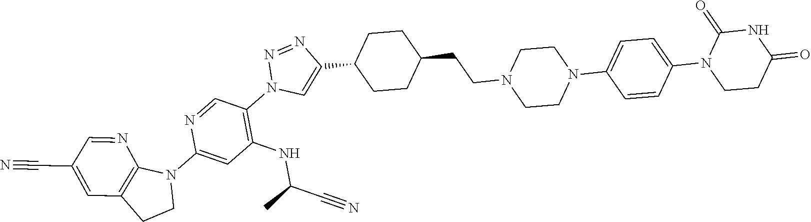 [SMILES: C[C@H](C#N)Nc1cc(N2CCc3cc(C#N)cnc32)ncc1-n1cc([C@H]2CC[C@H](CCN3CCN(c4ccc(N5CCC(=O)NC5=O)cc4)CC3)CC2)nn1] |
| P-487 | 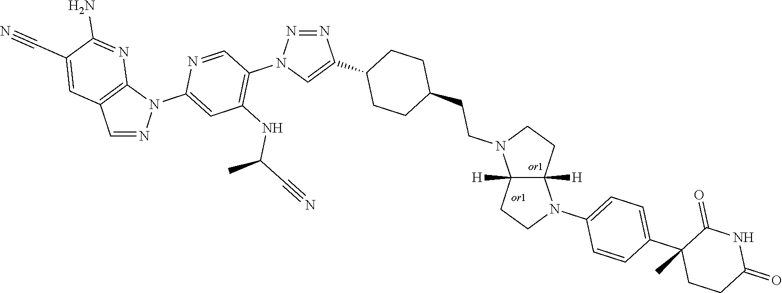 [SMILES: C[C@H](C#N)Nc1cc(-n2ncc3cc(C#N)c(N)nc32)ncc1-n1cc([C@H]2CC[C@H](CCN3CC[C@H]4[C@@H]3CCN4c3ccc([C@@]4(C)CCC(=O)NC4=O)cc3)CC2)nn1] |
| P-488 | 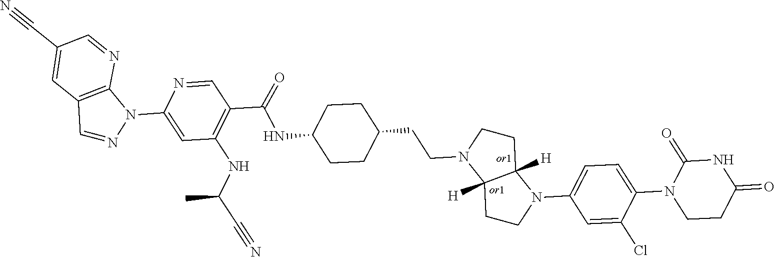 [SMILES: C[C@H](C#N)Nc1cc(-n2ncc3cc(C#N)cnc32)ncc1C(=O)N[C@H]1CC[C@@H](CCN2CC[C@H]3[C@@H]2CCN3c2ccc(N3CCC(=O)NC3=O)c(Cl)c2)CC1] |
| P-489 | 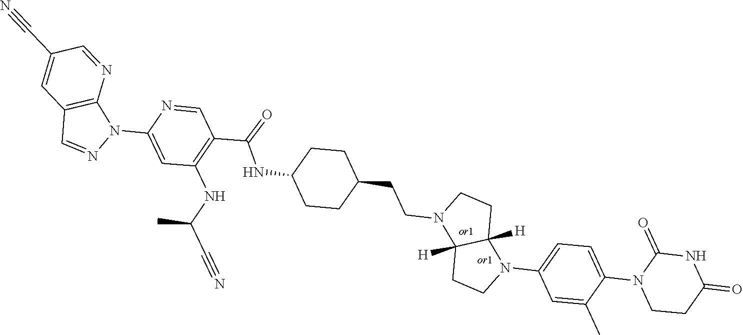 [SMILES: Cc1cc(N2CC[C@H]3[C@@H]2CCN3CC[C@H]2CC[C@H](NC(=O)c3cnc(-n4ncc5cc(C#N)cnc54)cc3N[C@H](C)C#N)CC2)ccc1N1CCC(=O)NC1=O] |
| P-490 | 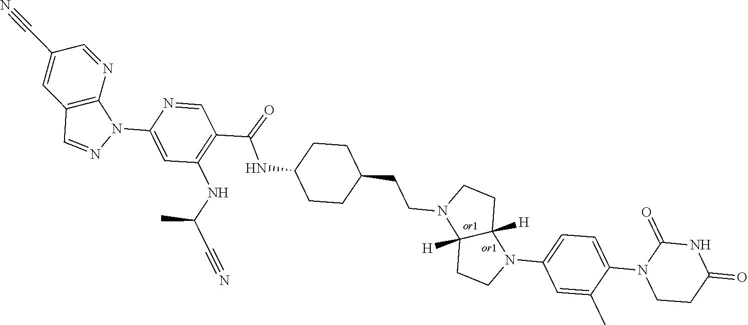 [SMILES: Cc1cc(N2CC[C@H]3[C@@H]2CCN3CC[C@H]2CC[C@H](NC(=O)c3cnc(-n4ncc5cc(C#N)cnc54)cc3N[C@H](C)C#N)CC2)ccc1N1CCC(=O)NC1=O] |
| P-491 | 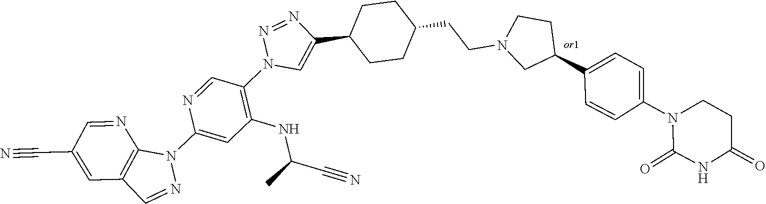 [SMILES: C[C@H](C#N)Nc1cc(-n2ncc3cc(C#N)cnc32)ncc1-n1cc([C@H]2CC[C@H](CCN3CC[C@@H](c4ccc(N5CCC(=O)NC5=O)cc4)C3)CC2)nn1] |
| P-492 | 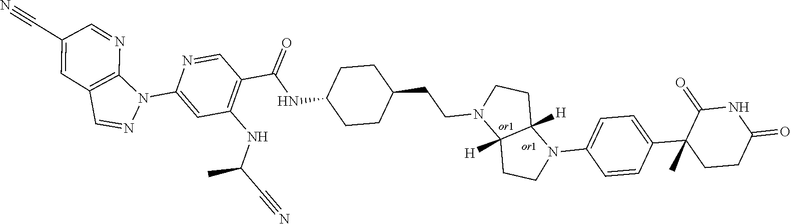 [SMILES: C[C@H](C#N)Nc1cc(-n2ncc3cc(C#N)cnc32)ncc1C(=O)N[C@H]1CC[C@H](CCN2CC[C@H]3[C@@H]2CCN3c2ccc([C@@]3(C)CCC(=O)NC3=O)cc2)CC1] |
| P-494 | 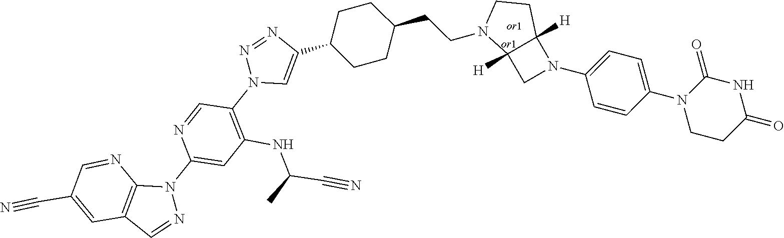 [SMILES: C[C@H](C#N)Nc1cc(-n2ncc3cc(C#N)cnc32)ncc1-n1cc([C@H]2CC[C@H](CCN3CC[C@H]4[C@@H]3CN4c3ccc(N4CCC(=O)NC4=O)cc3)CC2)nn1] |
| P-495 | 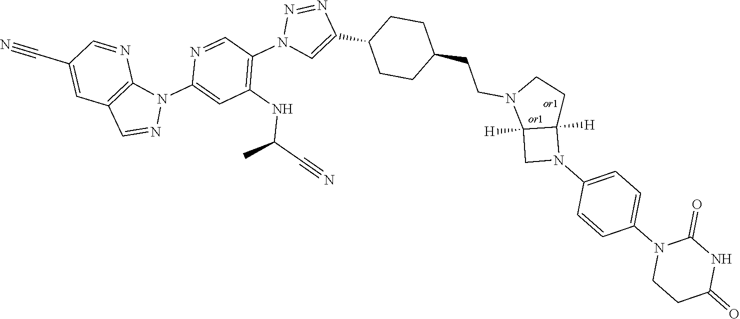 [SMILES: C[C@H](C#N)Nc1cc(-n2ncc3cc(C#N)cnc32)ncc1-n1cc([C@H]2CC[C@H](CCN3CC[C@@H]4[C@H]3CN4c3ccc(N4CCC(=O)NC4=O)cc3)CC2)nn1] |
| P-496 | 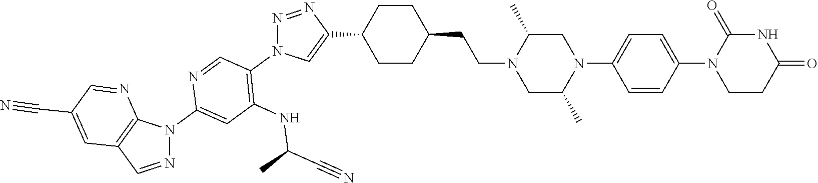 [SMILES: C[C@H](C#N)Nc1cc(-n2ncc3cc(C#N)cnc32)ncc1-n1cc([C@H]2CC[C@H](CCN3C[C@@H](C)N(c4ccc(N5CCC(=O)NC5=O)cc4)C[C@H]3C)CC2)nn1] |
| P-497 | 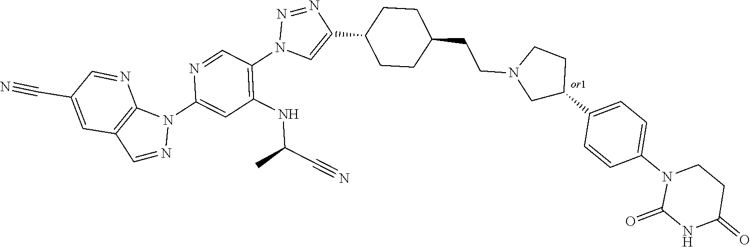 [SMILES: C[C@H](C#N)Nc1cc(-n2ncc3cc(C#N)cnc32)ncc1-n1cc([C@H]2CC[C@H](CCN3CC[C@H](c4ccc(N5CCC(=O)NC5=O)cc4)C3)CC2)nn1] |
| P-498 | 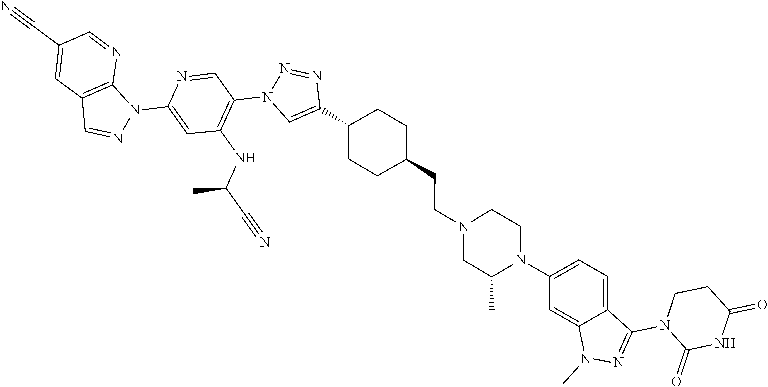 [SMILES: C[C@H](C#N)Nc1cc(-n2ncc3cc(C#N)cnc32)ncc1-n1cc([C@H]2CC[C@H](CCN3CCN(c4ccc5c(N6CCC(=O)NC6=O)nn(C)c5c4)[C@H](C)C3)CC2)nn1] |
| P-499 | 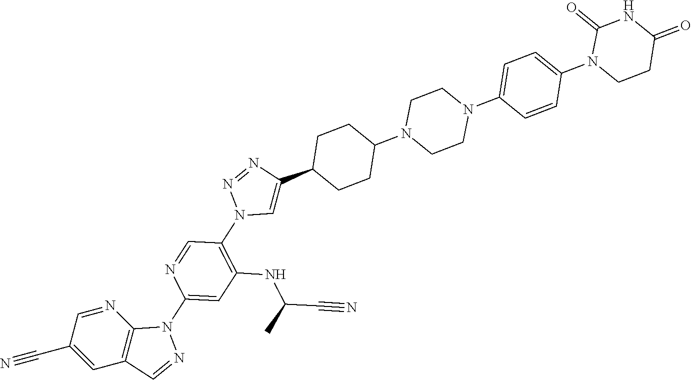 |
| P-500 | 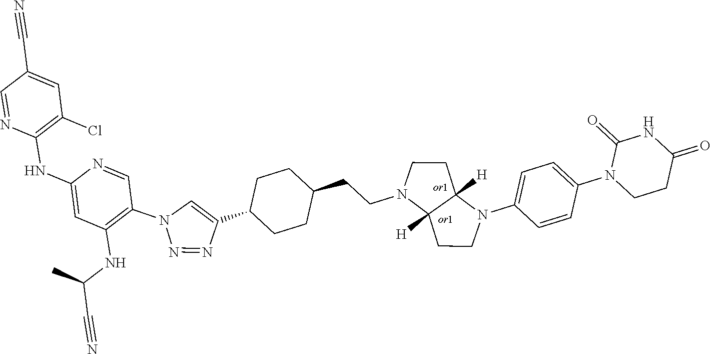 [SMILES: C[C@H](C#N)Nc1cc(Nc2ncc(C#N)cc2Cl)ncc1-n1cc([C@H]2CC[C@H](CCN3CC[C@H]4[C@@H]3CCN4c3ccc(N4CCC(=O)NC4=O)cc3)CC2)nn1] |
| P-501 | 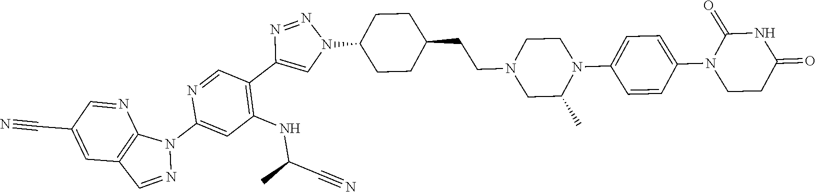 [SMILES: C[C@H](C#N)Nc1cc(-n2ncc3cc(C#N)cnc32)ncc1-c1cn([C@H]2CC[C@H](CCN3CCN(c4ccc(N5CCC(=O)NC5=O)cc4)[C@H](C)C3)CC2)nn1] |
| P-502 | 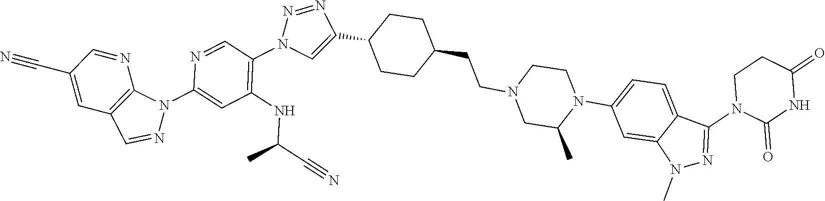 [SMILES: C[C@H](C#N)Nc1cc(-n2ncc3cc(C#N)cnc32)ncc1-n1cc([C@H]2CC[C@H](CCN3CCN(c4ccc5c(N6CCC(=O)NC6=O)nn(C)c5c4)[C@@H](C)C3)CC2)nn1] |
| P-503 | 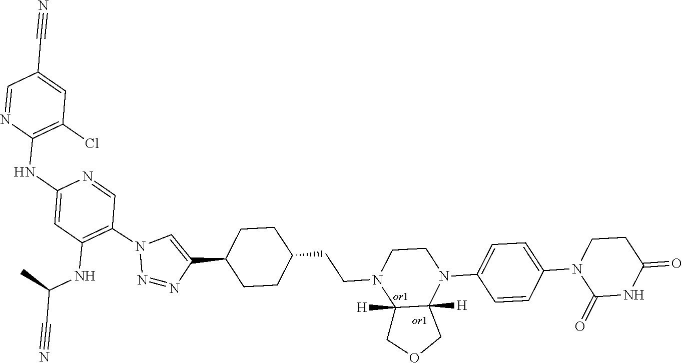 [SMILES: C[C@H](C#N)Nc1cc(Nc2ncc(C#N)cc2Cl)ncc1-n1cc([C@H]2CC[C@H](CCN3CCN(c4ccc(N5CCC(=O)NC5=O)cc4)[C@@H]4COC[C@@H]43)CC2)nn1] |
| P-504 | 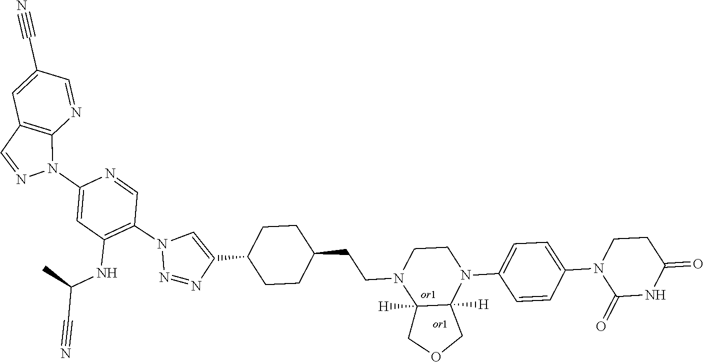 [SMILES: C[C@H](C#N)Nc1cc(-n2ncc3cc(CN)cnc32)ncc1-n1cc([C@H]2CC[C@H](CCN3CCN(c4ccc(N5CCC(=O)NC5=O)cc4)[C@H]4COC[C@H]43)CC2)nn1] |
| P-505 | 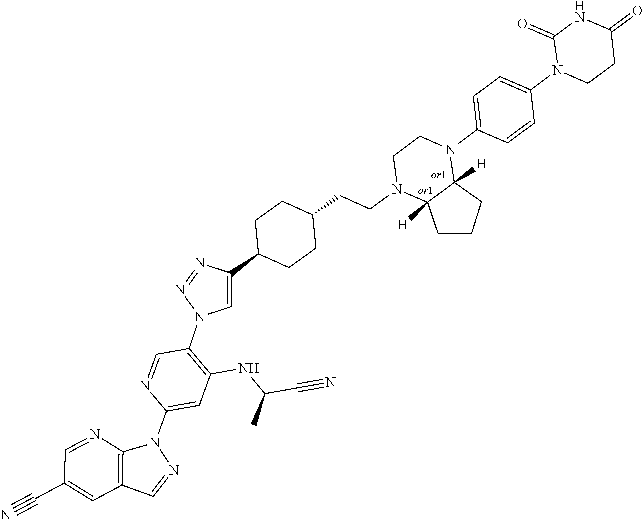 [SMILES: C[C@H](C#N)Nc1cc(-n2ncc3cc(CN)cnc32)ncc1-n1cc([C@H]2CC[C@H](CCN3CCN(c4ccc(N5CCC(=O)NC5=O)cc4)[C@@H]4CCC[C@@H]43)CC2)nn1] |
| P-506 | 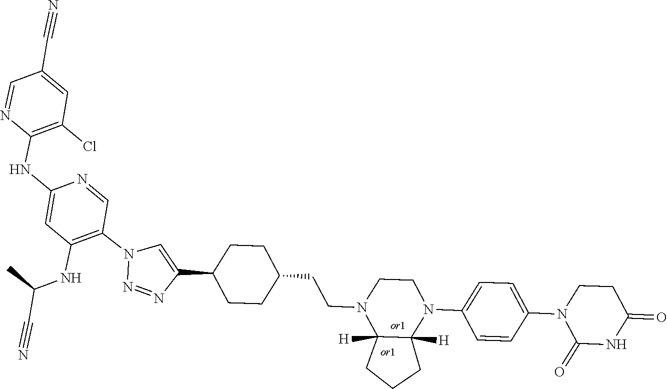 [SMILES: C[C@H](C#N)Nc1cc(Nc2ncc(C#N)cc2Cl)ncc1-n1cc([C@H]2CC[C@H](CCN3CCN(c4ccc(N5CCC(=O)NC5=O)cc4)[C@@H]4CCC[C@@H]43)CC2)nn1] |
| P-507 | 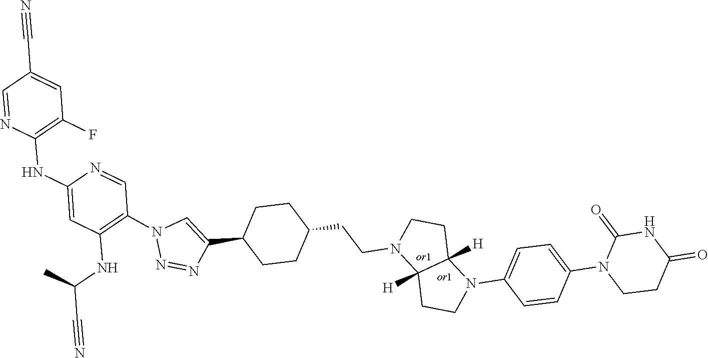 [SMILES: C[C@H](C#N)Nc1cc(Nc2ncc(C#N)cc2F)ncc1-n1cc([C@H]2CC[C@H](CCN3CC[C@H]4[C@@H]3CCN4c3ccc(N4CCC(=O)NC4=O)cc3)CC2)nn1] |
| P-508 | 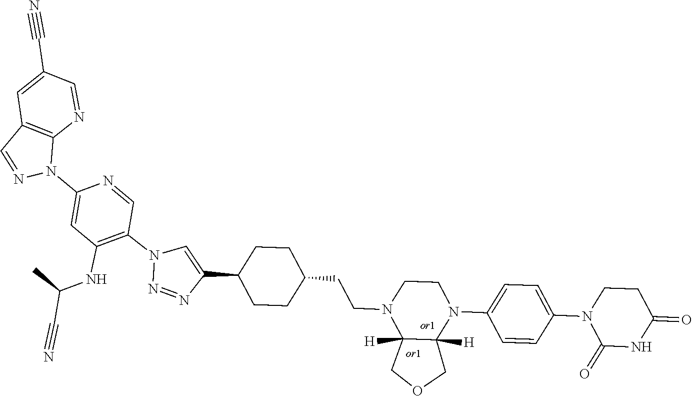 [SMILES: C[C@H](C#N)Nc1cc(-n2ncc3cc(CN)cnc32)ncc1-n1cc([C@H]2CC[C@H](CCN3CCN(c4ccc(N5CCC(=O)NC5=O)cc4)[C@@H]4COC[C@@H]43)CC2)nn1] |
| P-509 | 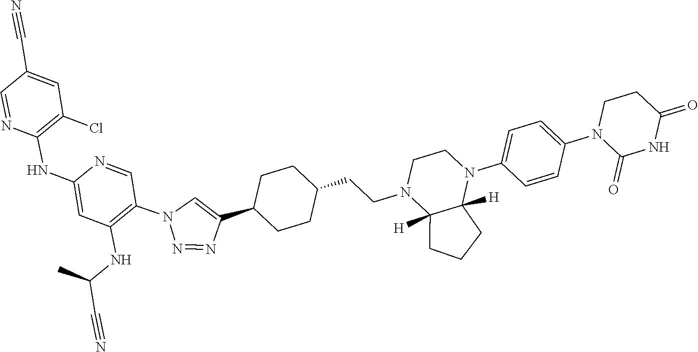 [SMILES: C[C@H](C#N)Nc1cc(Nc2ncc(C#N)cc2Cl)ncc1-n1cc([C@H]2CC[C@H](CCN3CCN(c4ccc(N5CCC(=O)NC5=O)cc4)[C@@H]4CCC[C@@H]43)CC2)nn1] |
| P-510 | 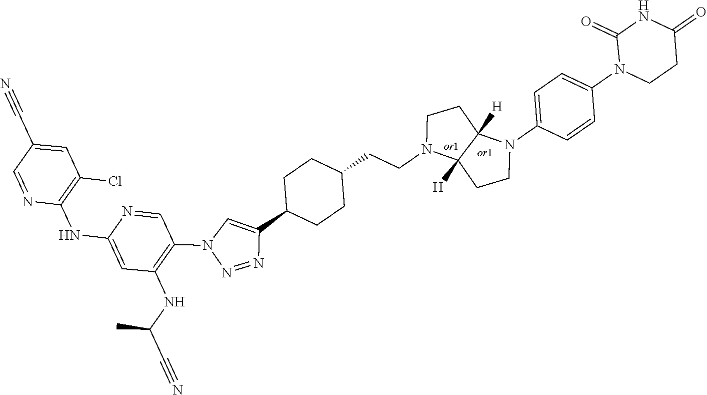 [SMILES: C[C@H](C#N)Nc1cc(Nc2ncc(C#N)cc2Cl)ncc1-n1cc([C@H]2CC[C@H](CCN3CC[C@H]4[C@@H]3CCN4c3ccc(N4CCC(=O)NC4=O)cc3)CC2)nn1] |
| P-511 | 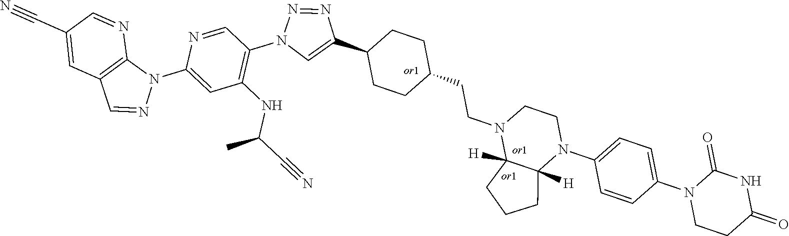 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)cc4)[C@@H]4CCC[C@@H]43)CC2)nn1] |
| P-512 | 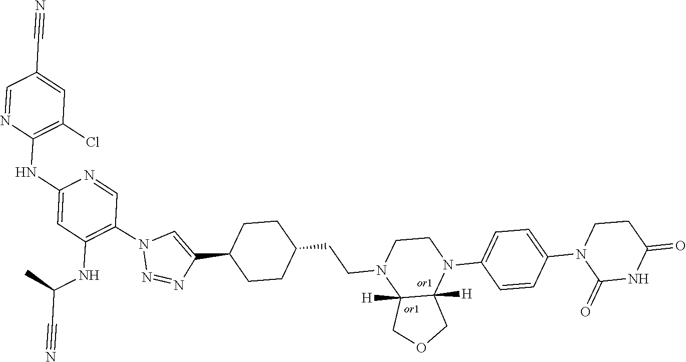 [SMILES: C[C@H](C#N)Nc1cc(Nc2ncc(C#N)cc2Cl)ncc1-n1cc([C@H]2CC[C@H](CCN3CCN(c4ccc(N5CCC(=O)NC5=O)cc4)[C@@H]4COC[C@@H]43)CC2)nn1] |
| P-513 | 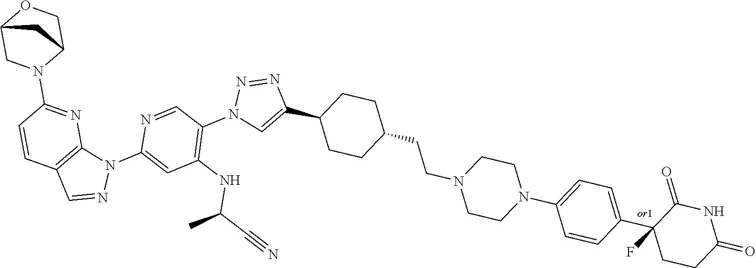 [SMILES: C[C@H](C#N)Nc1cc(-n2ncc3ccc(N4C[C@H]5C[C@@H]4CO5)nc32)ncc1-n1cc([C@H]2CC[C@H](CCN3CCN(c4ccc([C@@]5(F)CCC(=O)NC5=O)cc4)CC3)CC2)nn1] |

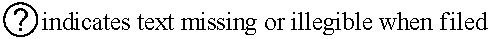

“or1” and “or2” indicates that the absolute stereochemistry was not determined and the stereochemistry may be as drawn or the opposite stereochemistry drawn.

It is understood that in the present description, combinations of substituents and/or variables of the depicted formulae are permissible only if such contributions result in stable compounds.

Furthermore, all compounds of Formula (I′) or (I) that exist in free base or acid form can be converted to their pharmaceutically acceptable salts by treatment with the appropriate inorganic or organic base or acid by methods known to one skilled in the art. Salts of the compounds of Formula (I′) or (I) can be converted to their free base or acid form by standard techniques.

### Methods of Synthesis

The compounds described herein can be made using conventional organic syntheses and commercially available starting materials, or the methods provided herein. By way of example and not limitation, compounds of Formula (I′) or (I) can be prepared as outlined in Scheme 1, as well as in the examples set forth herein. It should be noted that one skilled in the art would know how to modify the procedures set forth in the illustrative schemes and examples to arrive at the desired products.

Compounds of Formula (X-1) can be prepared as outlined in Scheme 1. Coupling of a Cereblon Binding Moiety (CBM) intermediate (C-X) with intermediate a under basic conditions with HATU forms intermediate b, which is then deprotected under acidic conditions to form intermediate c. Subsequent coupling of intermediate c with a target binding moiety (TBM) intermediate (A-X) affords compounds of Formula (X-1).

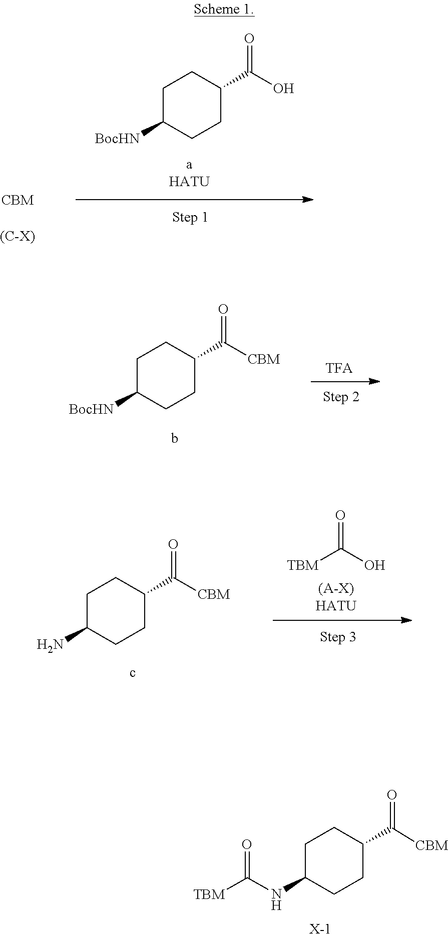

Compounds of Formula (X-2) can be prepared as outlined in Scheme 2. Coupling of a CBM intermediate (C-X) with intermediate d via reductive amination using NaBH(OAc)₃ affords intermediate e, which is then deprotected under acidic conditions to form intermediate f. Subsequent coupling of intermediate f with a TBM intermediate (A-X) provides compounds of Formula (X-2).

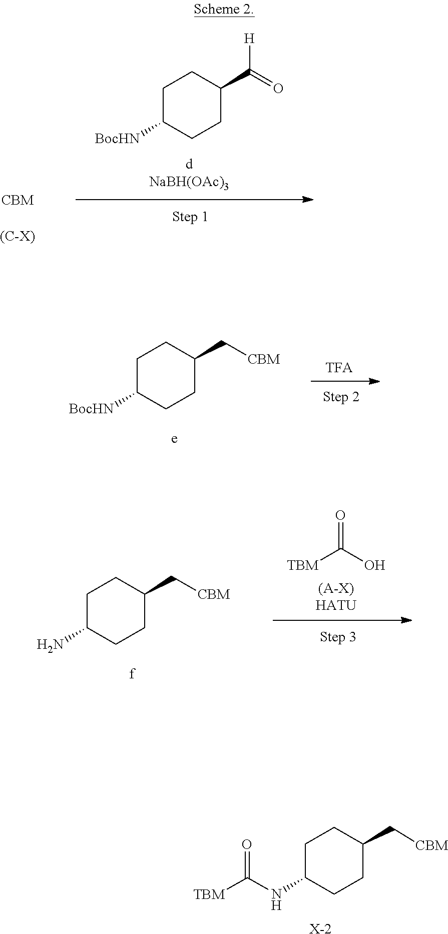

Compounds of Formula (X-3) can be prepared as outlined in Scheme 3. Activation of the alcohol of intermediate g to form mesylated intermediate h, followed by deoxyazidation with NaN₃ affords intermediate i. Intermediate i is then coupled with a TBM intermediate (A-X) to form intermediate j, which is then deprotected to form intermediate k. Subsequent coupling of intermediate k with a CBM intermediate (C-X) provides compounds of Formula (X-3).

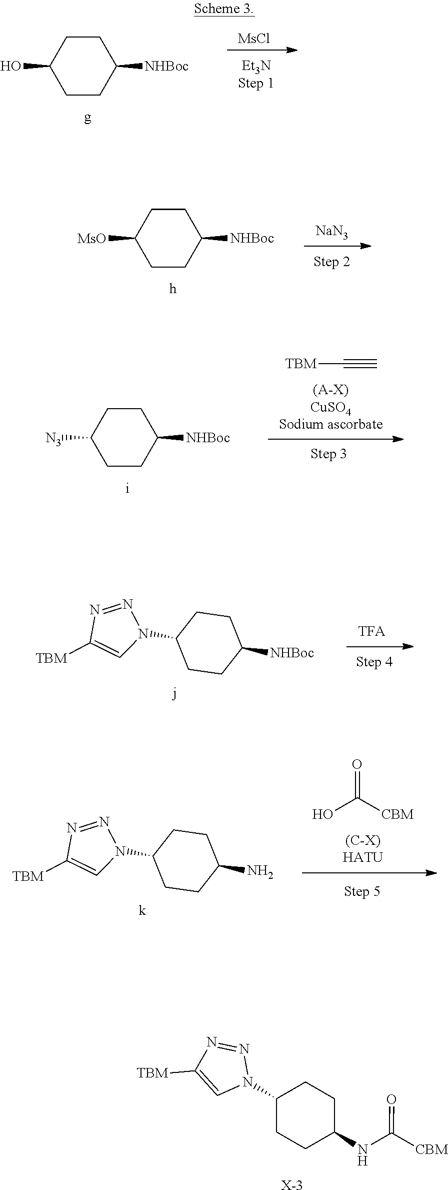

A synthetic route to compounds of Formula (X-4) is outlined in Scheme 4. Intermediate l can be coupled with intermediate m under basic conditions using HATU to form intermediate n, which is then activated with mesyl chloride to form intermediate o. Subsequent deoxyazidation with NaN₃ provides intermediate p. Deprotection of the ester of intermediate p affords intermediate q, which is then coupled with a CBM intermediate (C-X) to form intermediate f. Intermediate f is then coupled with TBM intermediate (A-X) via copper-catalyzed azide-alkyne cycloaddition to form compounds of Formula (X-4).

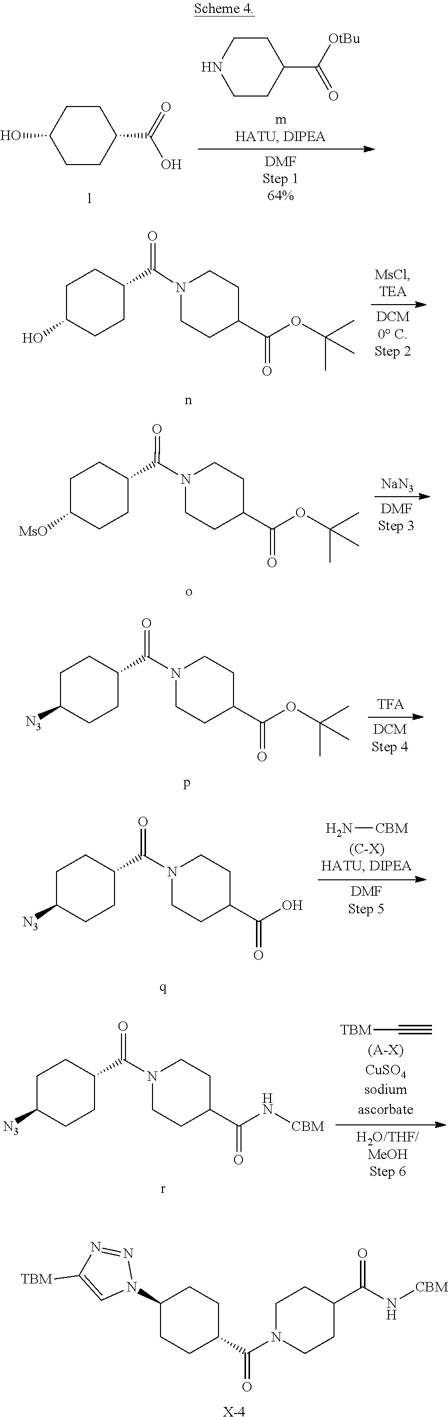

Compounds of Formula (X-7) can be prepared as outlined in Scheme 5. Activation of the alcohol of intermediate s with mesyl chloride forms intermediate t, and a deoxyazidation reaction with NaN₃ affords intermediate u. Reduction of the ester of intermediate u provides intermediate v, followed by activation with tosyl chloride to form intermediate w. Subsequent coupling of intermediate w with a CBM intermediate (C-X) under basic conditions affords intermediate x, which is then coupled with a TBM intermediate (A-X) via copper-catalyzed azide-alkyne cycloaddition to form compounds of Formula (X-7).

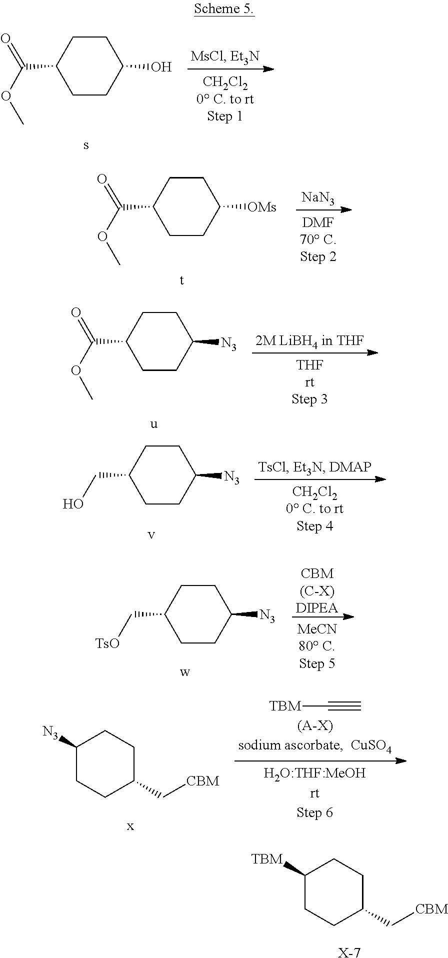

A synthetic route to compounds of Formula (X-8) is outlined in Scheme 6. Reduction of the carboxylic acid of intermediate y forms intermediate z, which is then oxidized to form intermediate a′. Subsequent coupling of intermediate a′ with a CBM intermediate (C-X) via reductive amination forms intermediate b′, which is then deprotected to form intermediate c′. Coupling of intermediate c′ with a TBM intermediate (A-X) via copper-catalyzed azide-alkyne cycloaddition yields compounds of Formula (X-8).

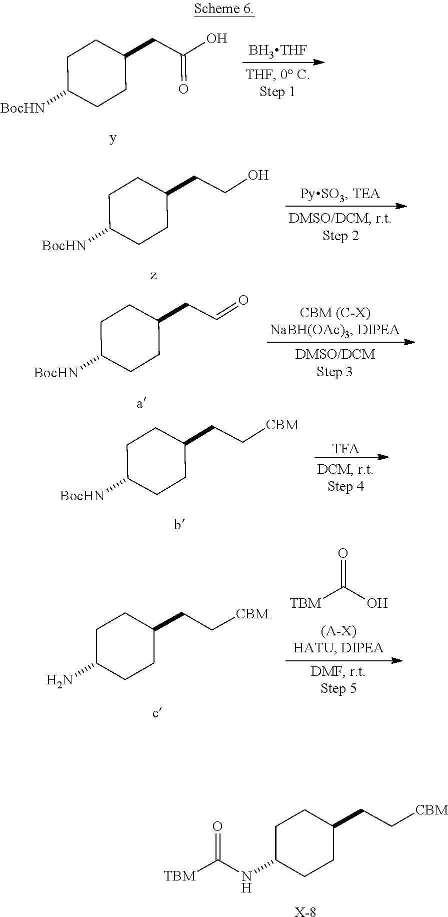

Compounds of Formula (X-11) can be prepared as outlined in Scheme 7. Protection of an alcohol group of intermediate c′ with TBDPS forms intermediate d′, which is then activated with mesyl chloride to form intermediate e′. The activated alcohol of intermediate e′ can be replaced with a cyano to form intermediate f, and then reduced to an aldehyde to form intermediate g′. Further reduction affords intermediate h′. The alcohol group of intermediate h′ can be protected with THP to form intermediate i′, and subsequent deprotection under acidic conditions forms intermediate j′. Intermediate j′ is then oxidized to form intermediate k′. The aldehyde of intermediate k′ can be converted to an alkyne via Seyferth-Gilbert homologation to form intermediate L-1, followed by deprotection of the alcohol to form intermediate L-2. Next, intermediate L-2 is coupled with a TBM intermediate (A-X) via copper-catalyzed azide-alkyne cycloaddition to form intermediate 1′, which is then coupled with a CBM intermediate (C-X) to form compounds of Formula (X-11).

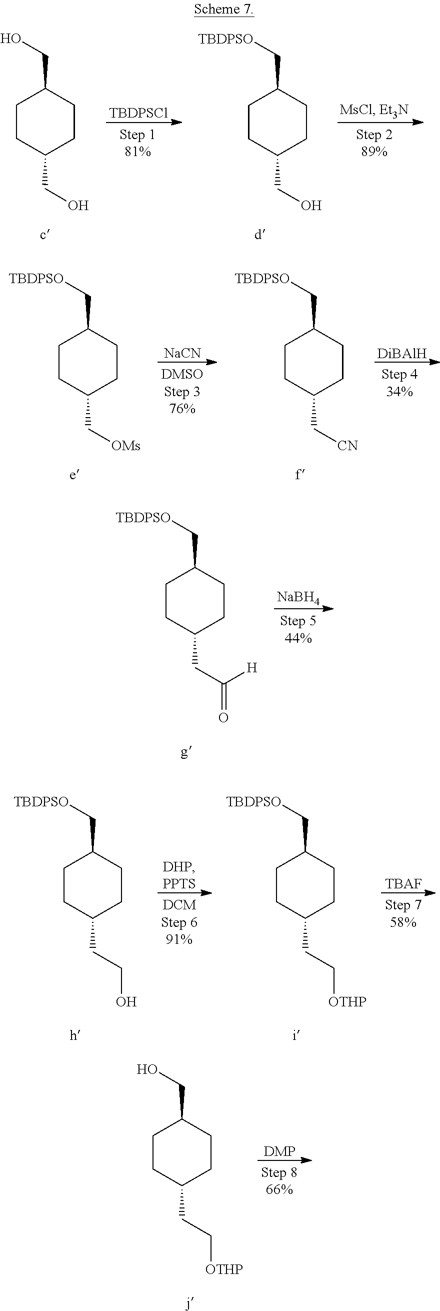

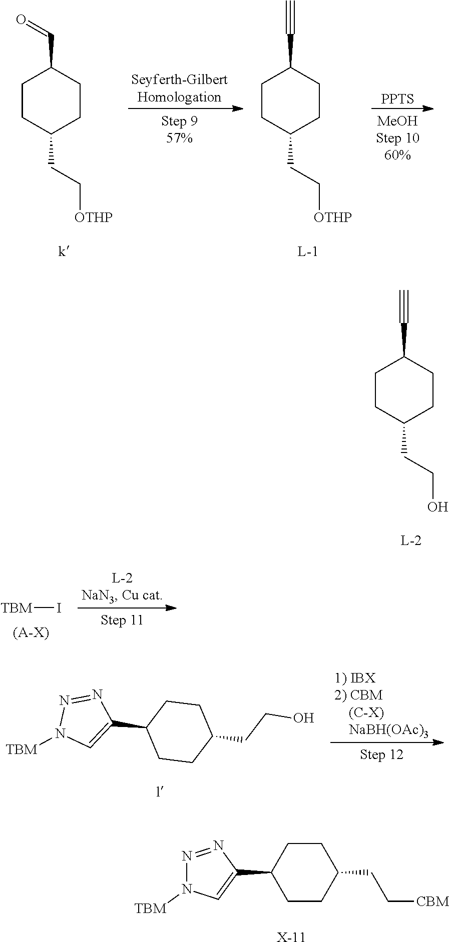

Compounds of Formula (X-15) can be prepared as outlined in Scheme 8. Oxidation of the alcohol group of intermediate m′ to an aldehyde affords intermediate n′, which is then coupled with intermediate o′ via reductive amination to form compounds of Formula (X-15).

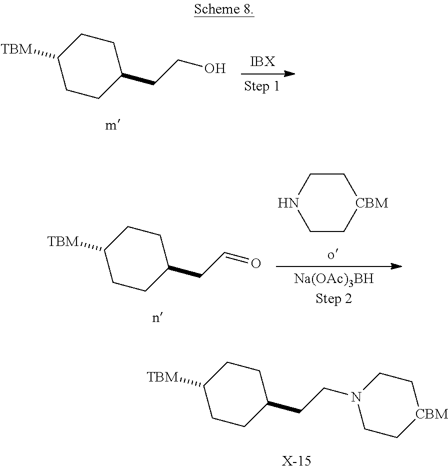

### Methods of Use

Embodiments of the present disclosure provide a method for modulating IRAK4 in a subject in need thereof, the method comprising administering to the subject an effective amount of a compound of Formula (I′) or (I). Modulation (e.g., inhibition or activation) of IRAK4 can be assessed and demonstrated by a wide variety of ways known in the art. Kits and commercially available assays can be utilized for determining whether and to what degree IRAK4 has been modulated (e.g., inhibited or activated).

In one aspect, provided herein is a method of modulating IRAK4 comprising contacting IRAK4 with an effective amount of a compound of Formula (I′) or (I) or any embodiment or variation thereof. In some embodiments, the compound of Formula (I′) or (I) inhibits IRAK4. In other embodiments, the compound of Formula (I′) or (I) activates IRAK4. In some embodiments, the compound of Formula (I′) or (I) is an agonist of IRAK4. In some embodiments, the compound of Formula (I′) or (I) is an antagonist of IRAK4.

In some embodiments, provided herein is a method for targeting IRAK4 for degradation comprising contacting IRAK4 with an effective amount of a compound of Formula (I′) or (I) or any embodiment or variation thereof.

In some embodiments, a compound of Formula (I′) or (I) modulates the activity of IRAK4 by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of Formula (I′) or (I) modulates the activity of IRAK4 by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

Also provided in certain embodiments of the present disclosure is a method for degrading IRAK4 in a subject in need thereof, the method comprising administering to the subject an effective amount of a compound of Formula (I′) or (I). Degradation of IRAK4 can be assessed and demonstrated by a wide variety of ways known in the art. Kits and commercially available assays, including cell-based assays, can be utilized for determining whether and to what degree IRAK4 has been degraded.

In one aspect, provided herein is a method of degrading IRAK4 comprising contacting IRAK4 with an effective amount of a compound of Formula (I′) or (I) or any embodiment or variation thereof. In some embodiments, the compound of Formula (I′) or (I) partially degrades IRAK3. In some embodiments, the compound of Formula (I′) or (I) fully degrades IRAK4.

In some embodiments, a compound of Formula (I′) or (I) degrades IRAK4 by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of Formula (I′) or (I) degrades IRAK4 by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 30-70%, or 40-60%.

In another aspect, provided herein is a method for treating an inflammatory or autoimmune disease in a subject in need thereof, comprising administering to the subject an effective amount of a compound of Formula (I′) or (I). In some embodiments, provided herein is a method for treating an inflammatory disease in a subject in need thereof, comprising administering to the subject an effective amount of a compound of Formula (I′) or (I). In some embodiments, provided herein is a method for treating an autoimmune disease in a subject in need thereof, comprising administering to the subject an effective amount of a compound of Formula (I′) or (I). In some embodiments, provided herein is a method for preventing an inflammatory or autoimmune disease in a subject in need thereof, comprising administering to the subject an effective amount of a compound of Formula (I′) or (I). In some embodiments, provided herein is a method for preventing an inflammatory disease in a subject in need thereof, comprising administering to the subject an effective amount of a compound of Formula (I′) or (I). In some embodiments, provided herein is a method for preventing an autoimmune disease in a subject in need thereof, comprising administering to the subject an effective amount of a compound of Formula (I′) or (I). Non-limiting examples of an inflammatory or autoimmune disease include atopic dermatitis, asthma, lupus, rheumatoid arthritis, familial mediterranean fever, psoriasis, generalized pustular psoriasis, cryoprin-associated periodic syndrome, hidradenitis suppurativa, Bechet's syndrome, or familial cold autoinflammatory syndrome.

In some embodiments, administering a compound of Formula (I′) or (I) to a subject that is predisposed to an inflammatory or autoimmune disease prevents the subject from developing any symptoms of the inflammatory or autoimmune disease. In some embodiments, administering a compound of Formula (I′) or (I) to a subject that does not yet display symptoms of an inflammatory or autoimmune disease prevents the subject from developing any symptoms of the inflammatory or autoimmune disease. In some embodiments, administering a compound of Formula (I′) or (I) to a subject in need thereof diminishes the extent of the inflammatory or autoimmune disease in the subject. In some embodiments, administering a compound of Formula (I′) or (I) to a subject in need thereof stabilizes the inflammatory or autoimmune disease (prevents or delays the worsening of the inflammatory or autoimmune disease). In some embodiments, administering a compound of Formula (I′) or (I) to a subject in need thereof delays the occurrence or recurrence of the inflammatory or autoimmune disease. In some embodiments, administering a compound of Formula (I′) or (I) to a subject in need thereof slows the progression of the inflammatory or autoimmune disease. In some embodiments, administering a compound of Formula (I′) or (I) to a subject in need thereof provides a partial remission of the inflammatory or autoimmune disease. In some embodiments, administering a compound of Formula (I′) or (I) to a subject in need thereof provides a total remission of the inflammatory or autoimmune disease. In some embodiments, administering a compound of Formula (I′) or (I) to a subject in need thereof decreases the dose of one or more other medications required to treat the inflammatory or autoimmune disease. In some embodiments, administering a compound of Formula (I′) or (I) to a subject in need thereof enhances the effect of another medication used to treat the inflammatory or autoimmune disease. In some embodiments, administering a compound of Formula (I′) or (I) to a subject in need thereof delays the progression of the inflammatory or autoimmune disease. In some embodiments, administering a compound of Formula (I′) or (I) to a subject in need thereof increases the quality of life of the subject having an inflammatory or autoimmune disease. In some embodiments, administering a compound of Formula (I′) or (I) to a subject in need thereof prolongs survival of a subject having an inflammatory or autoimmune disease.

In one aspect, provided herein is method of preventing a subject that is predisposed to an inflammatory or autoimmune disease from developing any symptoms of the inflammatory or autoimmune disease, the method comprising administering a compound of Formula (I′) or (I) to the subject. In some embodiments, provided herein is a method of preventing a subject that does not yet display symptoms of an inflammatory or autoimmune disease from developing any symptoms of the inflammatory or autoimmune disease, the method comprising administering a compound of Formula (I′) or (I) to the subject.

In some aspects, provided herein is a method of diminishing the extent of an inflammatory or autoimmune disease in a subject, the method comprising administering a compound of Formula (I′) or (I) to the subject. In some embodiments, provided herein is a method of stabilizing an inflammatory or autoimmune disease in a subject, the method comprising administering a compound of Formula (I′) or (I) to the subject. In some embodiments, the method prevents the worsening of the inflammatory or autoimmune disease. In some embodiments, the method delays the worsening of the inflammatory or autoimmune disease.

In another aspect, provided herein is a method of delaying the occurrence or recurrence of an inflammatory or autoimmune disease in a subject, the method comprising administering a compound of Formula (I′) or (I) to the subject.

In some embodiments, provided herein is a method of slowing the progression of an inflammatory or autoimmune disease in a subject, the method comprising administering a compound of Formula (I′) or (I) to the subject. In some embodiments, the method provides a partial remission of the inflammatory or autoimmune disease. In some embodiments, the method provides a total remission of the inflammatory or autoimmune disease.

In further aspects, provided herein is a method of decreasing the dose of one or more other medications required to treat an inflammatory or autoimmune disease in a subject, the method comprising administering a compound of Formula (I′) or (I) to the subject. In some embodiments, provided herein is a method of enhancing the effect of another medication used to treat an inflammatory or autoimmune disease in a subject, the method comprising administering a compound of Formula (I′) or (I) to the subject.

Also provided here is a method of delaying the progression of an inflammatory or autoimmune disease in a subject, the method comprising administering a compound of Formula (I′) or (I) to the subject. In some embodiments, the method increases the quality of life of the subject having an inflammatory or autoimmune disease. In some embodiments, the method prolongs survival of the subject having an inflammatory or autoimmune disease.

In another aspect, provided herein is a method for treating inflammatory or autoimmune symptoms caused by a disease in a subject in need thereof, comprising administering to the subject an effective amount of a compound of Formula (I′) or (I). In some embodiments, provided herein is a method for preventing inflammatory or autoimmune symptoms caused by a disease in a subject in need thereof, comprising administering to the subject an effective amount of a compound of Formula (I′) or (I). In some embodiments, administering a compound of Formula (I′) or (I) to a subject that is predisposed to a disease which causes inflammatory or autoimmune symptoms prevents the subject from developing any inflammatory or autoimmune symptoms. In some embodiments, administering a compound of Formula (I′) or (I) to a subject that does not yet display inflammatory or autoimmune symptoms of a disease which causes inflammatory or autoimmune symptoms prevents the subject from developing any inflammatory or autoimmune symptoms. In some embodiments, administering a compound of Formula (I′) or (I) to a subject in need thereof diminishes the extent of the inflammatory or autoimmune symptoms caused by the disease in the subject. In some embodiments, administering a compound of Formula (I′) or (I) to a subject in need thereof stabilizes the inflammatory or autoimmune symptoms of the disease (prevents or delays the worsening of the inflammatory or autoimmune symptoms). In some embodiments, administering a compound of Formula (I′) or (I) to a subject in need thereof delays the occurrence or recurrence of the inflammatory or autoimmune symptoms caused by the disease. In some embodiments, administering a compound of Formula (I′) or (I) to a subject in need thereof slows the progression of the inflammatory or autoimmune symptoms caused by the disease. In some embodiments, administering a compound of Formula (I′) or (I) to a subject in need thereof provides a partial remission of the disease which causes inflammatory or autoimmune symptoms. In some embodiments, administering a compound of Formula (I′) or (I) to a subject in need thereof provides a total remission of the disease which causes inflammatory or autoimmune symptoms. In some embodiments, administering a compound of Formula (I′) or (I) to a subject in need thereof decreases the dose of one or more other medications required to treat the disease which causes inflammatory or autoimmune symptoms. In some embodiments, administering a compound of Formula (I′) or (I) to a subject in need thereof enhances the effect of another medication used to treat the inflammatory or autoimmune symptoms of the disease. In some embodiments, administering a compound of Formula (I′) or (I) to a subject in need thereof delays the progression of the disease which causes inflammatory or autoimmune symptoms. In some embodiments, administering a compound of Formula (I′) or (I) to a subject in need thereof increases the quality of life of the subject having a disease which causes inflammatory or autoimmune symptoms. In some embodiments, administering a compound of Formula (I′) or (I) to a subject in need thereof prolongs survival of a subject having a disease which causes inflammatory or autoimmune symptoms. In some embodiments, the disease is atopic dermatitis, asthma, lupus, rheumatoid arthritis, familial mediterranean fever, psoriasis, generalized pustular psoriasis, cryoprin-associated periodic syndrome, hidradenitis suppurativa, Bechet's syndrome, or familial cold autoinflammatory syndrome.

In some embodiments, compounds of Formula (I′) or (I) are useful for treating a disorder selected from atopic dermatitis, asthma, lupus, rheumatoid arthritis, familial mediterranean fever, psoriasis, generalized pustular psoriasis, cryoprin-associated periodic syndrome, hidradenitis suppurativa, Bechet's syndrome, and familial cold autoinflammatory syndrome.

In some embodiments, compounds of Formula (I′) or (I) are useful for treating a cancer. In some embodiments, the cancer is a solid tumor, skin cancer, or lymphoma. In some embodiments, the cancer is squamous-cell carcinoma, basal cell carcinoma, adenocarcinoma, hepatocellular carcinoma, renal cell carcinoma, cancer of the bladder, bowel, breast, cervix, colon, esophagus, head, kidney, liver, lung, neck, ovary, pancreas, prostate, or stomach, leukemia, benign and malignant lymphomas, Burkitt's lymphoma, Non-Hodgkin's lymphoma, benign and malignant melanomas, myeloproliferative diseases, sarcomas, Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, Schwannomas, bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor, or teratocarcinomas. Additional cancers which may be treated using compounds of Formula (I′) or (I) include, for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, B-cell ALL, Philadelphia chromosome positive ALL and Philadelphia chromosome positive CML.

In some embodiments, the cancer is breast cancer, colorectal cancer, non-small cell lung cancer, ovarian, renal, sarcoma, melanoma, head and neck, hepatocellular, thyroid, multidrug-resistant leukemia, lymphoma, multiple myeloma, esophageal, large bowel, pancreatic, mesothelioma, carcinoma (e.g., adenocarcinoma, including esophageal adenocarcinoma), sarcoma (e.g., spindle cell sarcoma, liposarcoma, leiomyosarcoma, abdominal leiomyosarcoma, sclerosing epithelioid sarcoma) and melanoma (e.g., metastatic malignant melanoma).

In some embodiments, the compounds of Formula (I′) or (I) are useful for treating fibrosis, such as interstitial lung fibrosis, cystic fibrosis, progressive pulmonary fibrosis, and idiopathic pulmonary fibrosis.

### Pharmaceutical Compositions and Routes of Administration

The compounds provided herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions.

The compounds disclosed herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g, sodium benzoate, sodium bisulfate, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the compounds of Formula (I′) or (I) in the pharmaceutical composition may be at a level that will exercise the desired effect; for example, about 0.005 mg/kg of a subject's body weight to about 10 mg/kg of a subject's body weight in unit dosage for both oral and parenteral administration.

The dose of a compound of Formula (I′) or (I) to be administered to a subject is rather widely variable and can be subject to the judgment of a health-care practitioner. In general, the compounds disclosed herein can be administered one to four times a day in a dose of about 0.001 mg/kg of a subject's body weight to about 10 mg/kg of a subject's body weight, but the above dosage may be properly varied depending on the age, body weight and medical condition of the subject and the type of administration. In one embodiment, the dose is about 0.001 mg/kg of a subject's body weight to about 5 mg/kg of a subject's body weight, about 0.01 mg/kg of a subject's body weight to about 5 mg/kg of a subject's body weight, about 0.05 mg/kg of a subject's body weight to about 1 mg/kg of a subject's body weight, about 0.1 mg/kg of a subject's body weight to about 0.75 mg/kg of a subject's body weight or about 0.25 mg/kg of a subject's body weight to about 0.5 mg/kg of a subject's body weight. In one embodiment, one dose is given per day. In any given case, the amount of the compound of Formula (I′) or (I) administered will depend on such factors as the solubility of the active component, the formulation used and the route of administration.

In some embodiments, a compound of Formula (I′) or (I) is administered to a subject at a dose of about 0.01 mg/day to about 750 mg/day, about 0.1 mg/day to about 375 mg/day, about 0.1 mg/day to about 150 mg/day, about 0.1 mg/day to about 75 mg/day, about 0.1 mg/day to about 50 mg/day, about 0.1 mg/day to about 25 mg/day, or about 0.1 mg/day to about mg/day.

In another embodiment, provided herein are unit dosage formulations that comprise between about 0.1 mg and 500 mg, about 1 mg and 250 mg, about 1 mg and about 100 mg, about 1 mg and about 50 mg, about 1 mg and about 25 mg, or between about 1 mg and about 10 mg of a compound of Formula (I′) or (I).

In a particular embodiment, provided herein are unit dosage formulations comprising about 0.1 mg or 100 mg of a compound of Formula (I′) or (I).

In another embodiment, provided herein are unit dosage formulations that comprise mg, 1 mg, 5 mg, 10 mg, 15 mg, 20 mg, 30 mg, 35 mg, 50 mg, 70 mg, 100 mg, 125 mg, 140 mg, 175 mg, 200 mg, 250 mg, 280 mg, 350 mg, 500 mg, 560 mg, 700 mg, 750 mg, 1000 mg or 1400 mg of a compound of Formula (I′) or (I).

A compound of Formula (I′) or (I) can be administered once, twice, three, four or more times daily. In a particular embodiment, doses of 100 mg or less are administered as a once daily dose and doses of more than 100 mg are administered twice daily in an amount equal to one half of the total daily dose.

A compound of Formula (I′) or (I) can be administered orally for reasons of convenience. In one embodiment, when administered orally, a compound of Formula (I′) or (I) is administered with a meal and water. In another embodiment, the compound of Formula (I′) or (I) is dispersed in water or juice (e.g., apple juice or orange juice) or any other liquid and administered orally as a solution or a suspension.

The compounds disclosed herein can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend inpart upon the site of the medical condition.

In one embodiment, provided herein are capsules containing a compound of Formula (I′) or (I) without an additional carrier, excipient or vehicle.

In another embodiment, provided herein are compositions comprising an effective amount of a compound of Formula (I′) or (I) and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a compound of Formula (I′) or (I) with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the dye. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a compound of Formula (I′) or (I) as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the compound of Formula (I′) or (I) can be delayed or prolonged by proper formulation. For example, a slowly soluble pellet of the compound of Formula (I′) or (I) can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the compound of Formula (I′) or (I) in oily or emulsified vehicles that allow it to disperse slowly in the serum.

### Exemplary Embodiments

The present disclosure is further described by the following embodiments. The features of each of the embodiments are combinable with any of the other embodiments where appropriate and practical.

Embodiment 1. A compound of Formula (I′):

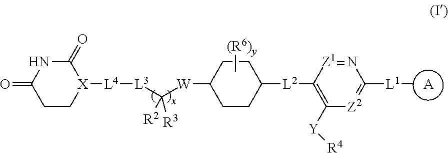

or a pharmaceutically acceptable salt thereof, wherein:
Ring A is phenyl, monocyclic 5- to 6-membered heteroaryl, or fused bicyclic 9- to 10-membered heteroaryl or heterocyclyl, wherein the heteroaryl and heterocyclyl contain 1-4 heteroatoms independently selected from N, O, and S, each of which is optionally substituted by 1-3 R⁰ groups;
each R⁰ is independently selected from halo, —CN, —NH₂, —NH(C₁-C₆ alkyl), C₁-C₆ alkyl, C₃-C₆ cycloalkyl, -(6- to 10-membered bridged heterocyclylene)-, and C₁-C₆ alkoxy, wherein the heterocyclylene contains 1-3 heteroatoms selected from N and O, or two R⁰ groups are taken together to form an oxo group;
L¹ is —NH— or a bond;
L² is —NHC(O)—, —C(O)NH—, —SO₂NH—, —NHSO₂—, or —(C₁-C₆ alkylene)_{z}(5-membered heteroarylene)-, wherein the heteroarylene contains 1-3 heteroatoms selected from N, O, and S;
L³ is —NR⁹ (C₁-C₆ alkylene)NR⁹—, —NR⁹C(O)(C₁-C₆ alkylene)_{z}(4- to 7-membered heterocyclylene)-, -(4- to 7-membered heterocyclylene)CR¹¹R¹²—, -(4- to 7-membered heterocyclylene)(CO)_{z}—, -(4- to 7-membered heterocyclylene)(NR⁹)_{z}—, —(NR⁹)_{z}(4- to 7-membered heterocyclylene)(C₁-C₆ alkylene)_{z}-, —NR⁹ (C₁-C₆ alkylene)_{z}(4- to 7-membered heterocyclylene)-, —NR⁹C(O)(phenylene)NR⁹—, —(C₁-C₆ alkylene)_{z}(4- to 7-membered heterocyclylene)(C₁-C₆ alkylene)_{z}-, —O(C₁-C₆ alkylene)_{z}(4- to 7-membered heterocyclylene)(C₁-C₆ alkylene)_{z}-, -(6- to 10-membered bridged heterocyclylene)(C₁-C₆ alkylene)_{z}-, -(7- to 10-membered fused bicyclic heterocyclylene)(C₁-C₆ alkylene)_{z}-, or —(O)_{z}(6- to 10-membered spiro heterocyclylene)(C₁-C₆ alkylene)_{z}-, wherein the heterocyclylene contains 1-3 heteroatoms selected from N and O and is optionally substituted by 1-5 R⁷ groups;
L⁴ is

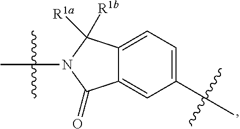

phenylene, —N(H)(phenylene), 5- to 6-membered heteroarylene, —N(H)(5- to 6-membered heteroarylene)-, 8- to 10-membered fused bicyclic heteroarylene, or 5- to 6-membered heterocyclylene, wherein the phenylene, heteroarylene, or heterocyclylene is optionally substituted by 1-4 R¹⁰ groups, and wherein the heteroarylene and heterocyclylene contain 1-3 heteroatoms selected from N, S, and O;
R^{1a} and R^{1b} are each H or are taken together to form an oxo group;
R² and R³ are independently H, C₁-C₆ alkyl, or halo, or R² and R³ are taken together to form an oxo group;
or R³ and R⁴ are taken together to form a C₃-C₆ cycloalkylene group;
Y is NH, O, or a bond;
R⁴ is C₃-C₆ cycloalkyl, C₁-C₆ alkylene-(C₃-C₆ cycloalkyl), 4- to 6-membered heterocyclyl, C₁-C₆ alkylene-(4- to 6-membered heterocyclyl), 5- to 6-membered heteroaryl, C₁-C₆ alkylene-(5- to 6-membered heteroaryl), C₁-C₆ alkyl, C₁-C₆ haloalkyl, C₁-C₆ alkyl-OH, or C₁-C₆ alkyl-CN, wherein the heterocyclyl and heteroaryl contain 1-3 heteroatoms selected from N and O, and wherein the cycloalkyl, heterocyclyl, or heteroaryl is optionally substituted by 1-5 R⁸ groups;
W is O, —NR⁵—, or a bond;
R⁵ is H or C₁-C₆ alkyl;
each R⁶ is independently C₁-C₆ alkyl, halo, or —OH, or two R⁶ groups are taken together to form a bridging C1-C₃ alkylene group;
each R⁷ is independently C₁-C₆ alkyl, halo, C₁-C₆ haloalkyl, or —OH, or two R⁷ groups are taken together to form an oxo group;
each R⁸ is independently —SO₂(C₁-C₆ alkyl), —C(O)(C₁-C₆ alkyl), C₁-C₆ alkyl, C₁-C₆ haloalkyl, halo, —CN, or —OH;
each R⁹ is independently H or C₁-C₆ alkyl;
each R¹⁰ is independently C₁-C₆ alkoxy, C₁-C₆ alkyl, halo, or —OH, or two R¹⁰ groups are taken together to form an oxo group;

each R¹¹ and R¹² is independently H, halo, C₃-C₆ cycloalkyl, —OH, —NH(C₁-C₆ alkyl), C₁-C₆ haloalkyl, or C₁-C₆ alkyl;
or R¹¹ and R³ are taken together to form a C₃-C₆ cycloalkylene group;
x is 0 or 1;
y is 0, 1, 2, 3, 4, or 5;
each z is independently 0 or 1;
X is N or CR¹³;
R¹³ is H, halo, —OH, or C₁-C₆ alkyl;
Z¹ is CH or N; and
Z² is CH or N,
provided that Z¹ and Z² are not both N.

Embodiment 2. A compound of Formula (I):

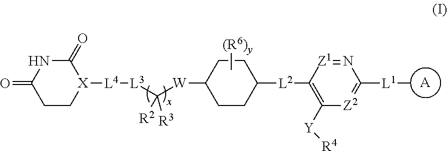

or a pharmaceutically acceptable salt thereof, wherein:
Ring A is phenyl, monocyclic 5- to 6-membered heteroaryl, or fused bicyclic 9- to 10-membered heteroaryl or heterocyclyl, wherein the heteroaryl and heterocyclyl contain 1-4 heteroatoms independently selected from N, O, and S, each of which is optionally substituted by 1-3 R⁰ groups;
each R⁰ is independently selected from halo, —CN, —NH₂, —NH(C₁-C₆ alkyl), C₁-C₆ alkyl, C₃-C₆ cycloalkyl, and C₁-C₆ alkoxy, or two R⁰ groups are taken together to form an oxo group;
L¹ is —NH— or a bond;
L² is —NHC(O)—, —C(O)NH—, —SO₂NH—, —NHSO₂—, or 5-membered heteroarylene containing 1-3 heteroatoms selected from N, O, and S;
L³ is —NR⁹ (C₁-C₆ alkylene)NR⁹—, —NR⁹C(O)(C₁-C₆ alkylene)_{z}(4- to 7-membered heterocyclylene)-, -(4- to 7-membered heterocyclylene)CR¹¹R¹²—, -(4- to 7-membered heterocyclylene)(CO)_{z}—, -(4- to 7-membered heterocyclylene)(NR⁹)_{z}—, —(NR⁹)_{z}(4- to 7-membered heterocyclylene)(C₁-C₆ alkylene)_{z}-, —NR⁹ (C₁-C₆ alkylene)_{z}(4- to 7-membered heterocyclylene)-, —NR⁹C(O)(phenylene)NR⁹—, —(C₁-C₆ alkylene)_{z}(4- to 7-membered heterocyclylene)-, —O(C₁-C₆ alkylene)_{z}(4- to 7-membered heterocyclylene)-, -(6- to 10-membered bridged heterocyclylene)(C₁-C₆ alkylene)_{z}-, -(9- to 10-membered fused bicyclic heterocyclylene)(C₁-C₆ alkylene)_{z}-, or —(O)_{z}(6- to 10-membered spiro heterocyclylene)(C₁-C₆ alkylene)_{z}-, wherein the heterocyclylene contains 1-3 heteroatoms selected from N and O and is optionally substituted by 1-5 R⁷ groups;
L⁴ is

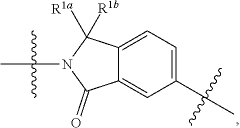

phenylene, 5- to 6-membered heteroarylene, or 5- to 6-membered heterocyclylene, wherein the phenylene, heteroarylene, or heterocyclylene is optionally substituted by 1-4 R¹⁰ groups, and wherein the heteroarylene and heterocyclylene contain 1-3 heteroatoms selected from N and O;
R^{1a} and R^{1b} are each H or are taken together to form an oxo group;
R² and R³ are independently H, C₁-C₆ alkyl, or halo, or R² and R³ are taken together to form an oxo group;
or R³ and R⁴ are taken together to form a C₃-C₆ cycloalkylene group; Y is NH or O;
R⁴ is C₃-C₆ cycloalkyl, C₁-C₆ alkylene-(C₃-C₆ cycloalkyl), 4- to 6-membered heterocyclyl, C₁-C₆ alkylene-(4- to 6-membered heterocyclyl), 5- to 6-membered heteroaryl, C₁-C₆ alkylene-(5- to 6-membered heteroaryl), C₁-C₆ alkyl, C₁-C₆ haloalkyl, C₁-C₆ alkyl-OH, or C₁-C₆ alkyl-CN, wherein the heterocyclyl and heteroaryl contain 1-3 heteroatoms selected from N and O, and wherein the cycloalkyl, heterocyclyl, or heteroaryl is optionally substituted by 1-5 R⁷ groups;
W is O, —NR⁵—, or a bond;
R⁵ is H or C₁-C₆ alkyl;
each R⁶ is independently C₁-C₆ alkyl, halo, or —OH, or two R⁶ groups are taken together to form a bridging C1-C₃ alkylene group;
each R⁷ is independently C₁-C₆ alkyl, halo, C₁-C₆ haloalkyl, or —OH, or two R⁷ groups are taken together to form an oxo group;
each R⁸ is independently —SO₂(C₁-C₆ alkyl), —C(O)(C₁-C₆ alkyl), C₁-C₆ alkyl, C₁-C₆ haloalkyl, halo, —CN, or —OH;
each R⁹ is independently H or C₁-C₆ alkyl;
each R¹⁰ is independently C₁-C₆ alkoxy, C₁-C₆ alkyl, halo, or —OH, or two R¹⁰ groups are taken together to form an oxo group;
each R¹¹ and R¹² is independently H, halo, C₃-C₆ cycloalkyl, —OH, —NH(C₁-C₆ alkyl), C₁-C₆ haloalkyl, or C₁-C₆ alkyl;
or R¹¹ and R³ are taken together to form a C₃-C₆ cycloalkylene group;
x is 0 or 1;
y is 0, 1, 2, 3, 4, or 5;
each z is independently 0 or 1;
X is N or CR¹³;
R¹³ is H, halo, or C₁-C₆ alkyl;
Z¹ is CH or N; and
Z² is CH or N,
provided that Z¹ and Z² are not both N.

Embodiment 3. The compound of embodiment 1 or 2, or a pharmaceutically acceptable salt thereof, wherein:
Z¹ and Z² are each CH.

Embodiment 4. The compound of embodiment 1 or 2, or a pharmaceutically acceptable salt thereof, wherein:
Z¹ is CH; and
Z² is N.

Embodiment 5. The compound of embodiment 1 or 2, or a pharmaceutically acceptable salt thereof, wherein:
Z¹ is N; and
Z² is CH.

Embodiment 6. The compound of any one of embodiments 1-5, or a pharmaceutically acceptable salt thereof, wherein:
Ring A is phenyl optionally substituted by 1-3 R⁰ groups; and
each R⁰ is independently selected from halo, —CN, —NH₂, —NH(C₁-C₃ alkyl), C₁-C₃ alkyl, C₃-C₆ cycloalkyl, and C₁-C₃ alkoxy.

Embodiment 7. The compound of embodiment 6, or a pharmaceutically acceptable salt thereof, wherein:

Ring A is

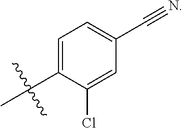 [SMILES: *c1ccc(C#N)cc1Cl]

Embodiment 8. The compound of any one of embodiments 1-5, or a pharmaceutically acceptable salt thereof, wherein:
Ring A is a monocyclic 6-membered heteroaryl containing 1-2 heteroatoms independently selected from N and O and is optionally substituted by 1-3 R⁰ groups; and each R⁰ is independently selected from halo, —CN, —NH₂, —NH(C₁-C₆ alkyl), C₁-C₆ alkyl, C₃-C₆ cycloalkyl, and C₁-C₆ alkoxy.

Embodiment 9. The compound of embodiment 8, or a pharmaceutically acceptable salt thereof, wherein:
Ring A is

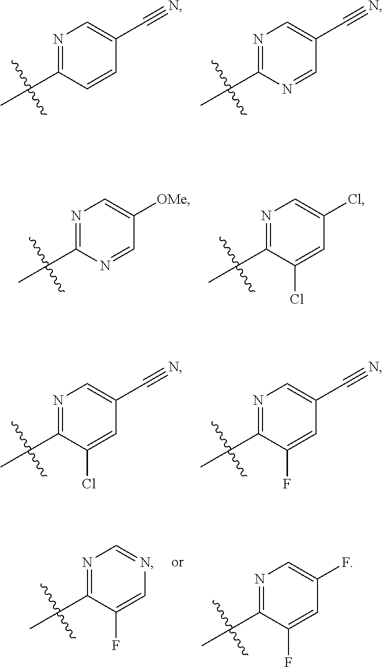

Embodiment 10. The compound of any one of embodiments 1-5, or a pharmaceutically acceptable salt thereof, wherein:
Ring A is a fused bicyclic 9- to 10-membered heteroaryl or heterocyclyl containing 2-4 heteroatoms independently selected from N, O, and S, and is optionally substituted by 1-3 R⁰ groups; and
each R⁰ is independently selected from halo, —CN, —NH₂, —NH(C₁-C₃ alkyl), C₁-C₃ alkyl, C₃-C₆ cycloalkyl, -(6- to 8-membered bridged heterocyclylene)-, and C₁-C₃ alkoxy, wherein the heterocyclylene contains 1-3 heteroatoms selected from N and O, or two R⁰ groups are taken together to form an oxo group.

Embodiment 11. The compound of embodiment 10, or a pharmaceutically acceptable salt thereof, wherein:
Ring A is

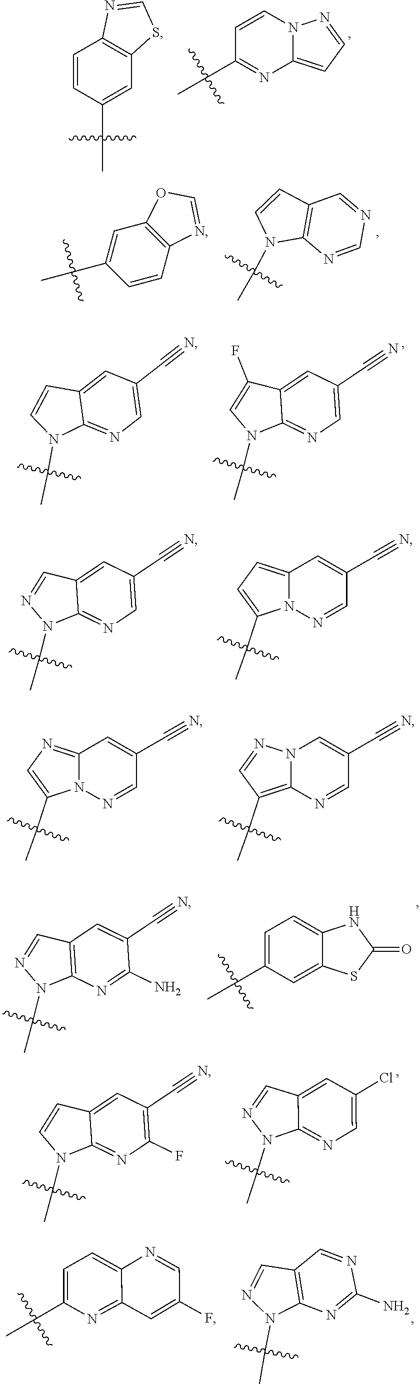

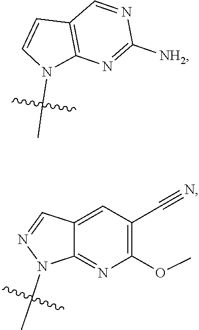 [SMILES: *n1ccc2cnc(N)nc21 ; *n1ncc2cc(C#N)c(OC)nc21]

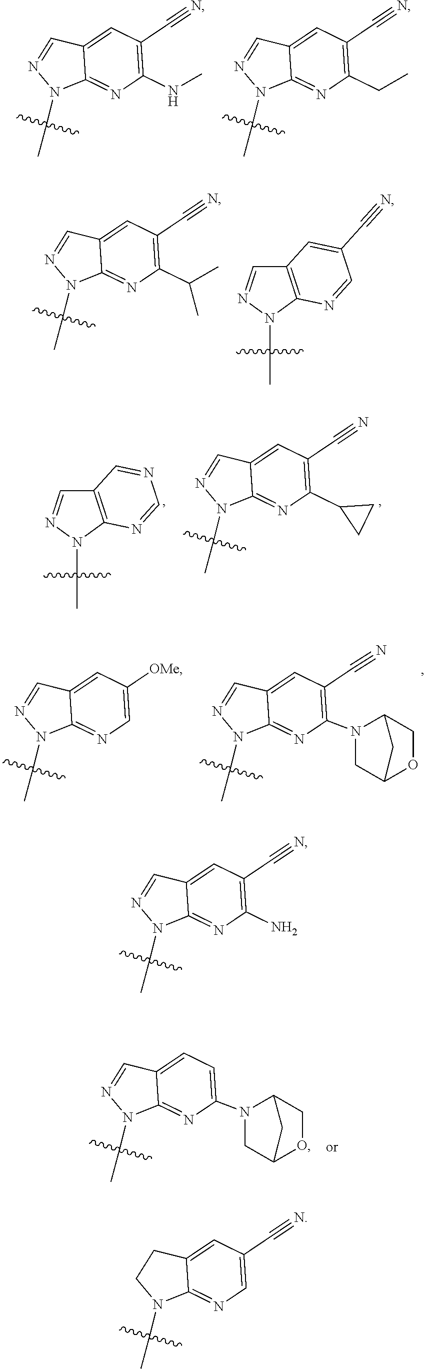

Embodiment 12. The compound of any one of embodiments 1-11, or a pharmaceutically acceptable salt thereof, wherein:
L¹ is —NH—.

Embodiment 13. The compound of any one of embodiments 1-11, or a pharmaceutically acceptable salt thereof, wherein:
L¹ is a bond.

Embodiment 14. The compound of any one of embodiments 1-13, or a pharmaceutically acceptable salt thereof, wherein:
L² is —(C₁-C₃ alkylene)_{z}(5-membered heteroarylene)-, wherein the heteroarylene contains 1-3 heteroatoms selected from N and O.

Embodiment 15. The compound of embodiment 14, or a pharmaceutically acceptable salt thereof, wherein:
L² is

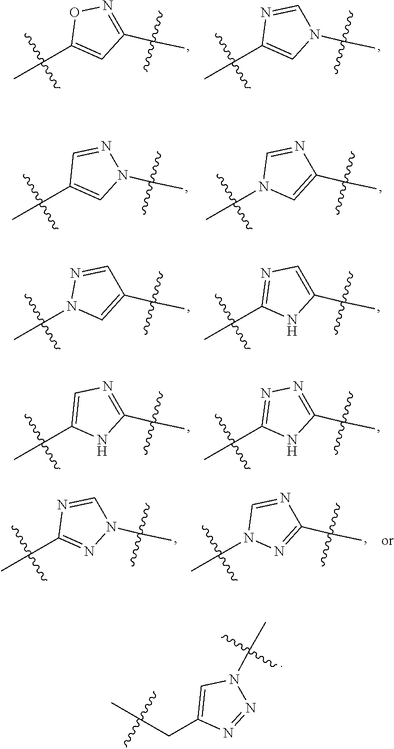

Embodiment 16. The compound of any one of embodiments 1-14, or a pharmaceutically acceptable salt thereof, wherein:
L² is —NHC(O)— or triazolylene.

Embodiment 17. The compound of embodiment 16, or a pharmaceutically acceptable salt thereof, wherein:
L² is —NHC(O)—.

Embodiment 18. The compound of embodiment 16, or a pharmaceutically acceptable salt thereof, wherein:
L² is

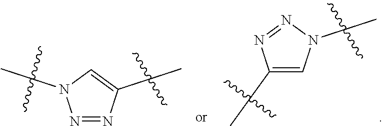 [SMILES: *c1cn(*)nn1 ; *c1cn(*)nn1]

Embodiment 19. The compound of any one of embodiments 1-18, or a pharmaceutically acceptable salt thereof, wherein:
L⁴ is

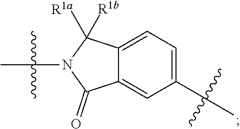

and
R^{1a} and R^{1b} are each H or are taken together to form an oxo group.

Embodiment 20. The compound of embodiment 19, or a pharmaceutically acceptable salt thereof, wherein:
L⁴ is

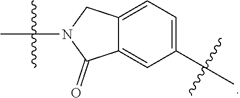 [SMILES: *c1ccc2c(c1)C(=O)N(*)C2]

Embodiment 21. The compound of embodiment 19, or a pharmaceutically acceptable salt thereof, wherein:
L⁴ is

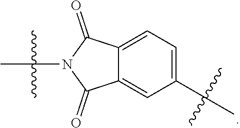 [SMILES: *c1ccc2c(c1)C(=O)N(*)C2=O]

Embodiment 22. The compound of any one of embodiments 1-18, or a pharmaceutically acceptable salt thereof, wherein:
L⁴ is phenylene, —NH(phenylene), 5- to 6-membered heteroarylene, —N(H)(5- to 6-membered heteroarylene)-, 8- to 10-membered fused bicyclic heteroarylene, or 5- to 6-membered heterocyclylene, each of which is optionally substituted by 1-4 R¹⁰ groups, and wherein the heteroarylene or heterocyclylene contains 1-3 heteroatoms selected from N, S, and O.

Embodiment 23. The compound of embodiment 22, or a pharmaceutically acceptable salt thereof, wherein:
each R¹⁰ is independently C₁-C₃ alkoxy, C₁-C₃ alkyl, halo, or —OH, or two R¹⁰ groups are taken together to form an oxo group.

Embodiment 24. The compound of embodiment 23, or a pharmaceutically acceptable salt thereof, wherein:
each R¹⁰ is independently —OCH₃, —CH₃, Cl, F, or —OH, or two R¹⁰ groups are taken together to form an oxo group.

Embodiment 25. The compound of any one of embodiments 22-24, or a pharmaceutically acceptable salt thereof, wherein:
L⁴ is

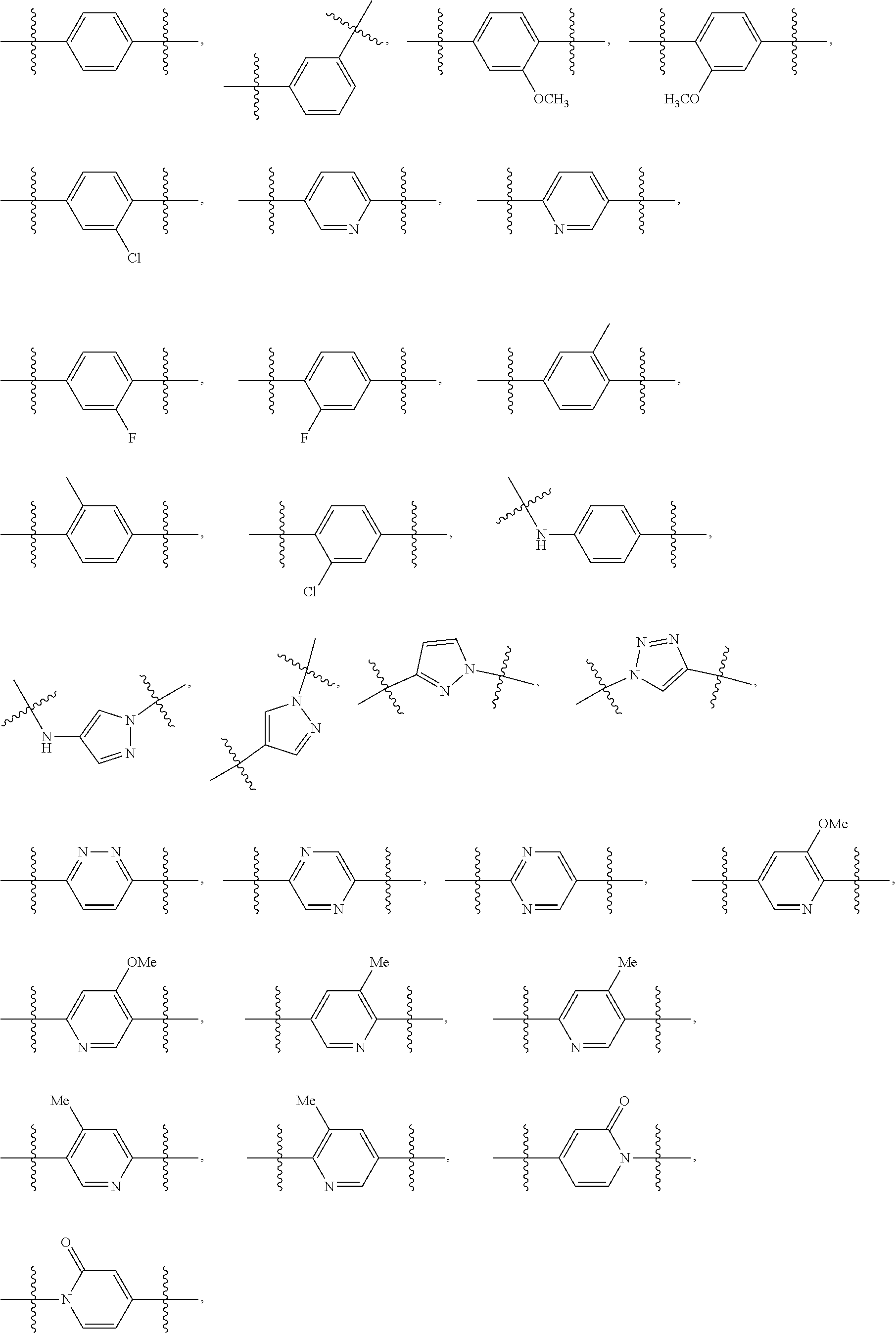

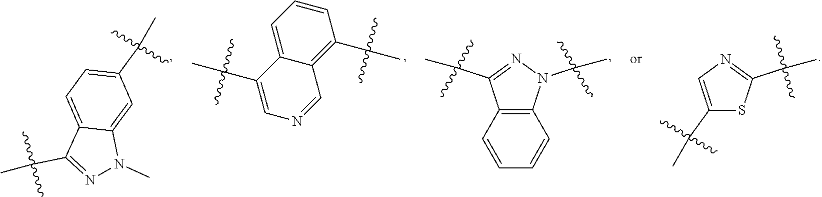 [SMILES: *c1ccc2c(*)nn(C)c2c1 ; *c1cncc2c(*)cccc12 ; *c1nn(*)c2ccccc12 ; *c1cnc(*)s1]

Embodiment 26. The compound of any one of embodiments 1-25, or a pharmaceutically acceptable salt thereof, wherein:
x is O.

Embodiment 27. The compound of any one of embodiments 1-25, or a pharmaceutically acceptable salt thereof, wherein:
x is 1.

Embodiment 28. The compound of any one of embodiments 1-25 and 27, or a pharmaceutically acceptable salt thereof, wherein:
R² and R³ are independently H, C₁-C₃ alkyl, or halo.

Embodiment 29. The compound of embodiment 28, or a pharmaceutically acceptable salt thereof, wherein:
R² and R³ are independently H, —CH₃, or F.

Embodiment 30. The compound of any one of embodiments 1-25 and 27, or a pharmaceutically acceptable salt thereof, wherein:
R² and R³ are taken together to form an oxo group.

Embodiment 31. The compound of any one of embodiments 1-25 and 27, or a pharmaceutically acceptable salt thereof, wherein:
R³ and R¹¹ are taken together to form a C₃-C₅ cycloalkylene group.

Embodiment 32. The compound of embodiment 31, or a pharmaceutically acceptable salt thereof, wherein:
R³ and R¹¹ are taken together to form a cyclopropylene group.

Embodiment 33. The compound of any one of embodiments 1-32, or a pharmaceutically acceptable salt thereof, wherein:
y is 0 or 1.

Embodiment 34. The compound of any one of embodiments 1-33, or a pharmaceutically acceptable salt thereof, wherein:
each R⁶ is independently C₁-C₃ alkyl, halo, or —OH, or two R⁶ groups are taken together to form a bridging C1-C₂ alkylene group.

Embodiment 35. The compound of embodiment 34, or a pharmaceutically acceptable salt thereof, wherein:
each R⁶ is independently —CH₃, C₁, or —OH, or two R⁶ groups are taken together to form a bridging C1-C₂ alkylene group.

Embodiment 36. The compound of any one of embodiments 1-35, or a pharmaceutically acceptable salt thereof, wherein:

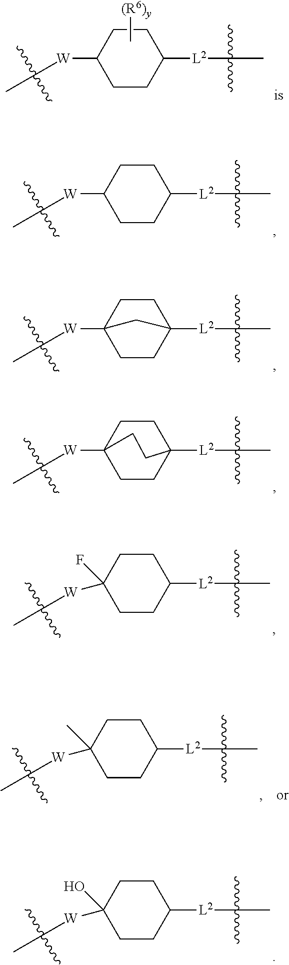

Embodiment 37. The compound of any one of embodiments 1-36, or a pharmaceutically acceptable salt thereof, wherein:
W is O, —NR⁵—, or a bond; and
R⁵ is H or C₁-C₃ alkyl.

Embodiment 38. The compound of embodiment 37, or a pharmaceutically acceptable salt thereof, wherein:
W is O, —N(H)—, —N(CH₃)—, or a bond.

Embodiment 39. The compound of any one of embodiments 1-38, or a pharmaceutically acceptable salt thereof, wherein:
X is N.

Embodiment 40. The compound of any one of embodiments 1-38, or a pharmaceutically acceptable salt thereof, wherein:
X is CR¹³; and
R¹³ is H, halo, —OH, or C₁-C₃ alkyl.

Embodiment 41. The compound of embodiment 40, or a pharmaceutically acceptable salt thereof, wherein:
R¹³ is H, CH₃, —OH, or F.

Embodiment 42. The compound of any one of embodiments 1-41, or a pharmaceutically acceptable salt thereof, wherein:
Y is NH.

Embodiment 43. The compound of any one of embodiments 1-41, or a pharmaceutically acceptable salt thereof, wherein:
Y is O or a bond.

Embodiment 44. The compound of any one of embodiments 1-43, or a pharmaceutically acceptable salt thereof, wherein:
R⁴ is C₃-C₆ cycloalkyl, C₁-C₃ alkylene-(C₃-C₆ cycloalkyl), 4- to 6-membered heterocyclyl, C₁-C₃ alkylene-(4- to 6-membered heterocyclyl), 5- to 6-membered heteroaryl, C₁-C₃ alkylene-(5- to 6-membered heteroaryl), C₁-C₆ alkyl, C₁-C₆ haloalkyl, C₁-C₆ alkyl-OH, or C₁-C₆ alkyl-CN, wherein the heterocyclyl and heteroaryl contain 1 or 2 heteroatoms selected from N and O, and wherein the cycloalkyl, heterocyclyl, or heteroaryl is optionally substituted by 1-2 R⁸ groups.

Embodiment 45. The compound of embodiment 44, or a pharmaceutically acceptable salt thereof, wherein:
each R⁸ is independently —SO₂(C₁-C₃ alkyl), —C(O)(C₁-C₃ alkyl), C₁-C₃ alkyl, C₁-C₃ haloalkyl, halo, —CN, or —OH.

Embodiment 46. The compound of embodiment 45, or a pharmaceutically acceptable salt thereof, wherein:
each R⁸ is independently —SO₂CH₃, —C(O)CH₃, methyl, ethyl, —CH₂CF₃, F, —CN, or —OH.

Embodiment 47. The compound of any one of embodiments 1-46, or a pharmaceutically acceptable salt thereof, wherein:
R⁴ is methyl, ethyl, n-propyl, isopropyl, tert-butyl, —CH₂CH(CH₃)₂, —CH₂CF₃, —CH₂CH₂F, —CH₂CF₂CH₃, —CH(CH₃)CF₃, —CH₂CH₂CF₃, —CH(CH₃)CH₂OH, —CH₂C(CH₃)₂OH, —CH₂CN, —CH(CH₃)CN, —C(CH₃)₂CN, —CH(CH₂CH₃)CN, —CH₂CH(CH₃)CN,

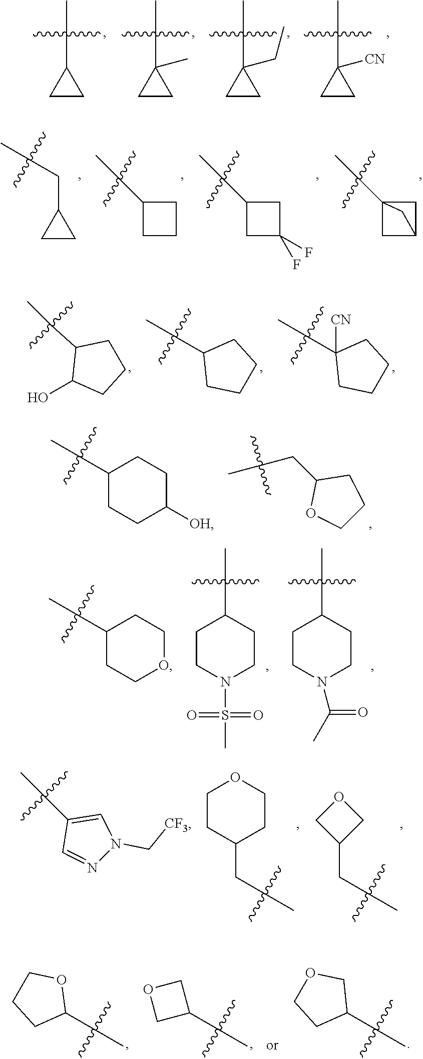

Embodiment 48. The compound of any one of embodiments 1-47, or a pharmaceutically acceptable salt thereof, wherein:
L³ is —NR⁹ (C₁-C₃ alkylene)NR⁹—, —NR⁹C(O)(C₁-C₃ alkylene)_{z}(4- to 7-membered heterocyclylene)-, -(4- to 7-membered heterocyclylene)CR¹¹R¹²—, -(4- to 7-membered heterocyclylene)(CO)_{z}—, -(4- to 7-membered heterocyclylene)(NR⁹)_{z}—, —(NR⁹)_{z}(4- to 7-membered heterocyclylene)(C₁-C₃ alkylene)_{z}-, —NR⁹ (C₁-C₃ alkylene)_{z}(4- to 7-membered heterocyclylene)-, —NR⁹C(O)(phenylene)NR⁹—, —(C₁-C₃ alkylene)_{z}(4- to 7-membered heterocyclylene)(C₁-C₃ alkylene)_{z}-, —O(C₁-C₃ alkylene)_{z}(4- to 7-membered heterocyclylene)(C₁-C₃ alkylene)_{z}-, -(6- to 10-membered bridged heterocyclylene)(C₁-C₃ alkylene)_{z}-, -(7- to 10-membered fused bicyclic heterocyclylene)(C₁-C₆ alkylene)_{z}-, or —(O)_{z}(6- to 10-membered spiro heterocyclylene)(C₁-C₃ alkylene)_{z}-, wherein the heterocyclylene contains 1-3 heteroatoms selected from N and O and is optionally substituted by 1 or 2 R⁷ groups; and each z is independently 0 or 1.

Embodiment 49. The compound of any one of embodiments 1-48, or a pharmaceutically acceptable salt thereof, wherein:
each R⁹ is independently H or C₁-C₃ alkyl.

Embodiment 50. The compound of embodiment 49, or a pharmaceutically acceptable salt thereof, wherein:
each R⁹ is independently H or —CH₃.

Embodiment 51. The compound of any one of embodiments 1-50, or a pharmaceutically acceptable salt thereof, wherein:
each R⁷ is independently C₁-C₃ alkyl, halo, C₁-C₃ haloalkyl, or —OH, or two R⁷ groups are taken together to form an oxo group.

Embodiment 52. The compound of embodiment 51, or a pharmaceutically acceptable salt thereof, wherein:
each R⁷ is independently —CH₃, —CF₃, F, or —OH, or two R⁷ groups are taken together to form an oxo group.

Embodiment 53. The compound of any one of embodiments 1-52, or a pharmaceutically acceptable salt thereof, wherein:
each R¹¹ and R¹² is independently H or C₁-C₃ alkyl.

Embodiment 54. The compound of embodiment 53, or a pharmaceutically acceptable salt thereof, wherein:
each R¹¹ and R¹² is independently H or —CH₃.

Embodiment 55. The compound of any one of embodiments 1-48, or a pharmaceutically acceptable salt thereof, wherein:
L³ is

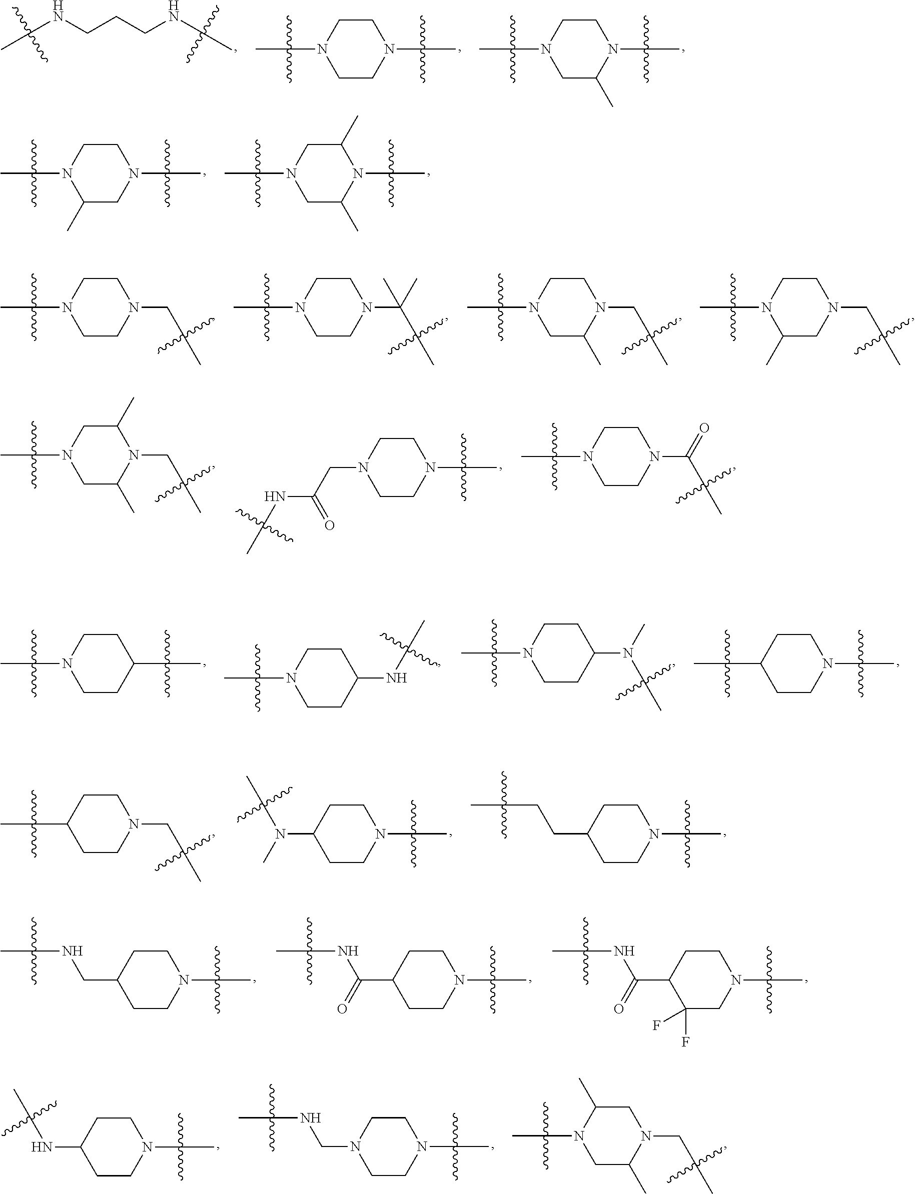

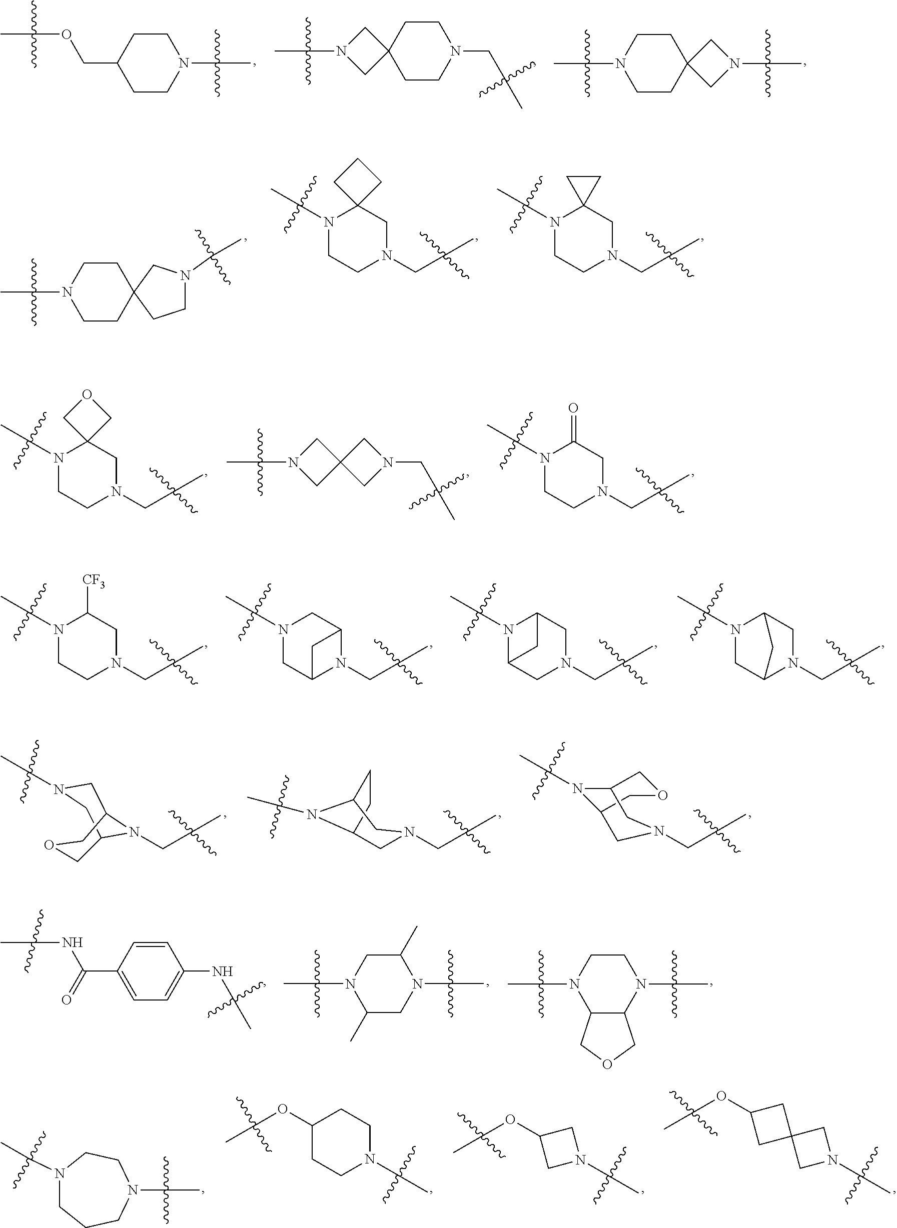

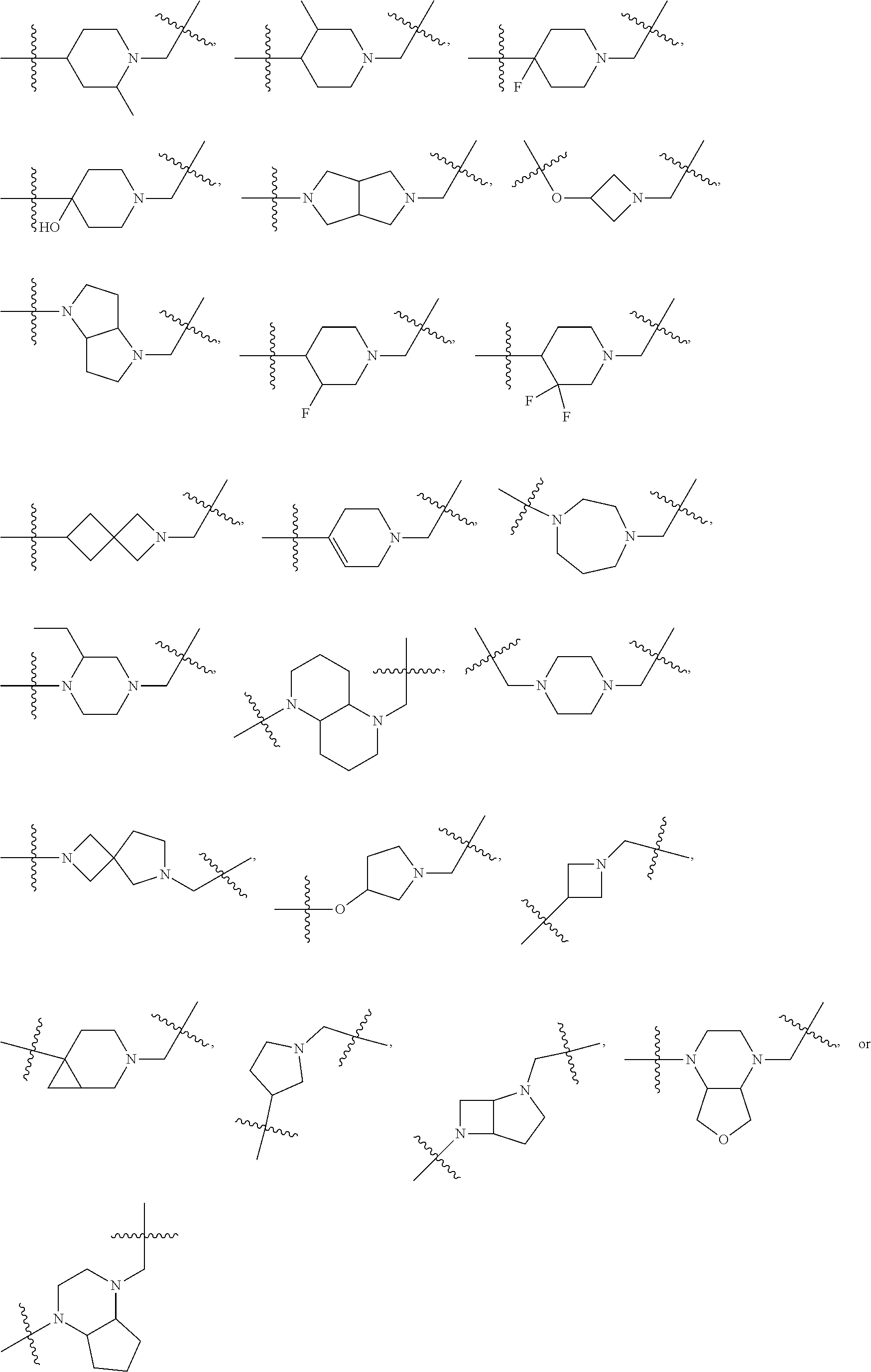

Embodiment 56. The compound of any one of embodiments 1-3, 10, 11, 13-18, 22-27-38, 40-42, 44-48, and 51-55, or a pharmaceutically acceptable salt thereof, wherein the compound is of Formula (Ia):
wherein:

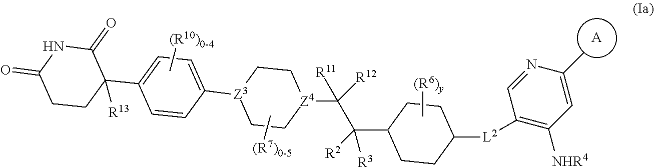

Ring A is a fused bicyclic 9- to 10-membered heteroaryl containing 2-4 heteroatoms independently selected from N, O, and S, optionally substituted by 1-3 R⁰ groups; and
Z³ and Z⁴ are independently N or CH, provided that at least one of Z³ and Z⁴ is N.

Embodiment 57. The compound of embodiment 56, or a pharmaceutically acceptable salt thereof, wherein the compound is of Formula (Ib) or (Ic):

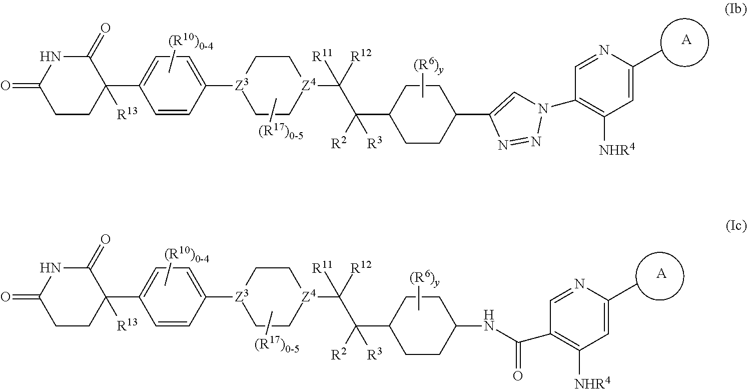

Embodiment 58. The compound of embodiment 56 or 57, or a pharmaceutically acceptable salt thereof, wherein the compound is of Formula (Id) or (Ie):

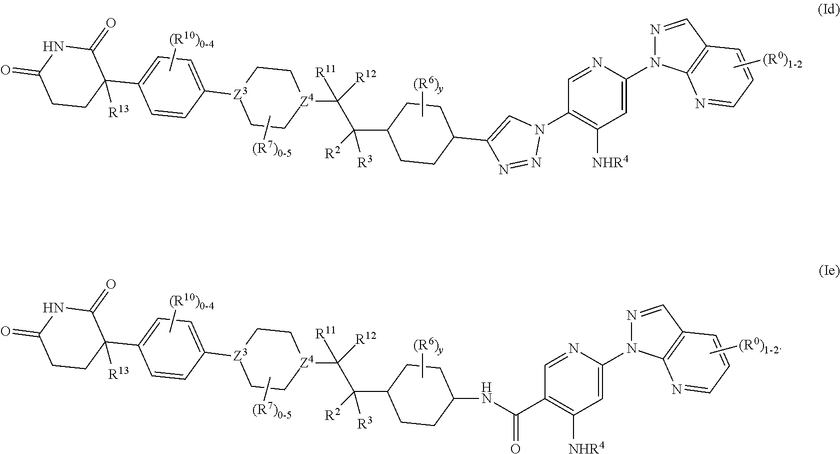

Embodiment 59. The compound of any one of embodiments 56-58, or a pharmaceutically acceptable salt thereof, wherein the compound is of Formula (If) or (Ig):

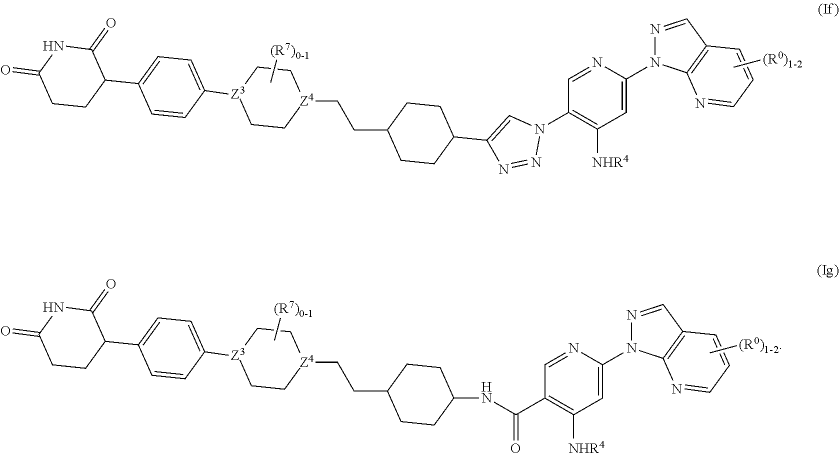

Embodiment 60. The compound of embodiment 59, or a pharmaceutically acceptable salt thereof, wherein:
each R⁰ is independently —CN or —NH₂;
R⁴ is C₁-C₃ alkyl-CN, or C₃-C₆ cycloalkyl optionally substituted with C₁-C₃ alkyl; and
R⁷ is C₁-C₃ alkyl.

Embodiment 61. The compound of any one of embodiments 1, 2, 10, 11, 13-18, 22-27-38, 39, 42, 44-48, and 51-54, or a pharmaceutically acceptable salt thereof, wherein the compound is of Formula (Ia′):
wherein:

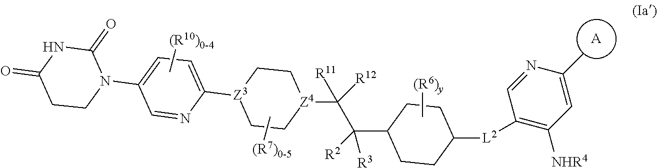

Ring A is a fused bicyclic 9- to 10-membered heteroaryl containing 2-4 heteroatoms independently selected from N, O, and S, optionally substituted by 1-3 R⁰ groups; and
Z³ and Z⁴ are independently N or CH, provided that at least one of Z³ and Z⁴ is N.

Embodiment 62. The compound of embodiment 61, or a pharmaceutically acceptable salt thereof, wherein the compound is of Formula (Ib′) or (Ic′):

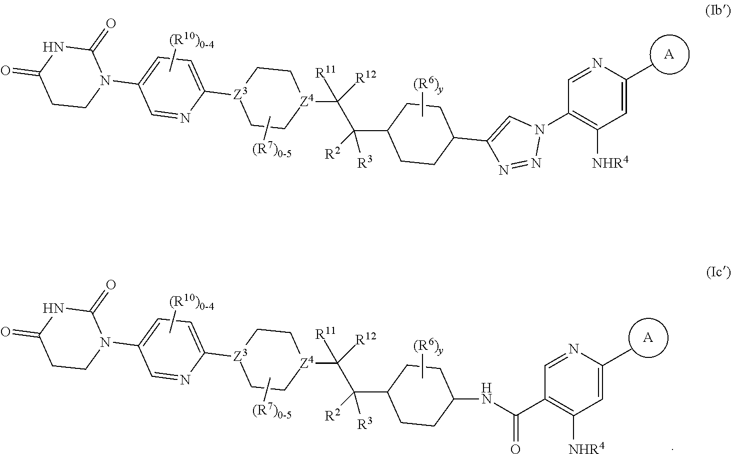

Embodiment 63. The compound of embodiment 61 or 62, or a pharmaceutically acceptable salt thereof, wherein the compound is of Formula (Id′) or (Ie′):

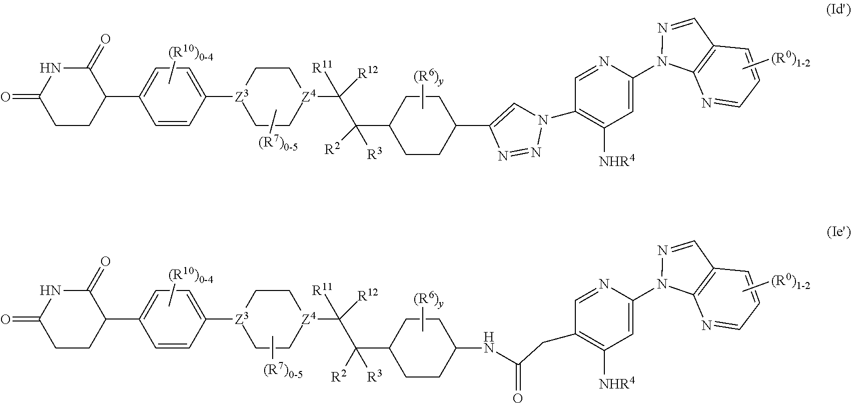

Embodiment 64. The compound of any one of embodiments 61-63, or a pharmaceutically acceptable salt thereof, wherein the compound is of Formula (If′) or (Ig′):

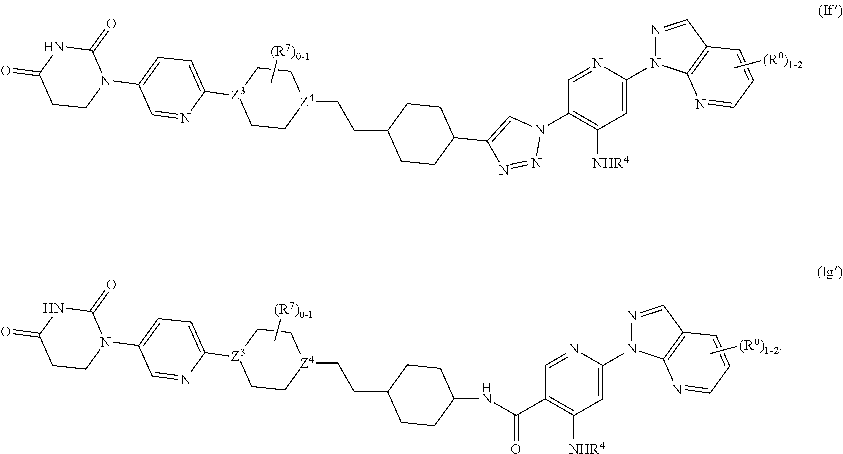

Embodiment 65. The compound of embodiment 64, or a pharmaceutically acceptable salt thereof, wherein:
each R⁰ is independently —CN or —NH₂;
R⁴ is C₁-C₃ alkyl-CN, or C₃-C₆ cycloalkyl optionally substituted with C₁-C₃ alkyl; and
R⁷ is C₁-C₃ alkyl.

Embodiment 66. A compound selected from the compounds of Table 1 or a pharmaceutically acceptable salt thereof.

Embodiment 67. A pharmaceutical composition comprising the compound of any one of embodiments 1-66, or a pharmaceutically acceptable salt thereof, and a pharmaceutically acceptable excipient.

Embodiment 68. A method of modulating interleukin-1 (IL1) receptor-associated kinase 4 (IRAK4) activity comprising contacting IRAK4 with an effective amount of the compound of any one of embodiments 1-66, or a pharmaceutically acceptable salt thereof, or the pharmaceutical composition of embodiment 67.

Embodiment 69. A method of treating an inflammatory or autoimmune disease in a subject in need thereof, comprising administering to the subject an effective amount of the compound of any one of embodiments 1-66, or a pharmaceutically acceptable salt thereof, or the pharmaceutical composition of embodiment 67.

Embodiment 70. The method of embodiment 69, wherein the inflammatory or autoimmune disease is atopic dermatitis, asthma, lupus, rheumatoid arthritis, familial mediterranean fever, psoriasis, generalized pustular psoriasis, cryoprin-associated periodic syndrome, hidradenitis suppurativa, Bechet's syndrome, or familial cold autoinflammatory syndrome.

### Examples

The following Examples are presented by way of illustration, not limitation. Compounds are named using the automatic name generating tool provided in ChemBiodraw Ultra (Cambridgesoft), which generates systematic names for chemical structures, with support for the Cahn-Ingold-Prelog rules for stereochemistry. One skilled in the art can modify the procedures set forth in the illustrative examples to arrive at the desired products.

Salts of the compounds described herein can be prepared by standard methods, such as inclusion of an acid (for example TFA, formic acid, or HCl) in the mobile phases during chromatography purification, or stirring of the products after chromatography purification, with a solution of an acid (for example, aqueous HCl).

As used in certain of the chemical structures provided in the following Examples, designation of a particular atom with “*”, “or1”, or “or2” indicates that the absolute stereochemistry of the indicated atom was not determined.

The following abbreviations may be relevant for the application.

### Abbreviations

ACN or MeCN: acetonitrile
ADMP: 2-azido-1,3-dimethylimidazolinium hexafluorophosphate
aq: aqueous
BCA assay: Bicinchoninic acid assay
CBM: cereblon binding moiety
CV: column volume
d: day(s)
DABCO: 1,4-diazabicyclo[2.2.2]octane
DAST: diethylaminosulfur trifluoride
DCM: dichloromethane
DIPEA: N, N-diisopropylethylamine
DMA: dimethylacetamide
DMAP: 4-dimethylaminopyridine
DMAP: 4-dimethylaminopyridine
DME: dimethoxyethane
DMF: dimethylformamide
DMP: Dess-Martin Periodinane
EDCI: 1-ethyl-3-(3-dimethylaminopropyl)carbodiimide
Equiv or eq.: equivalents
ESI: electrospray ionization
EtOAc: ethyl acetate
FA: formic acid
FBS: fetal bovine serum
FC: flash chromatography
h: hour(s)
HATU: 1-[bis(dimethylamino)methylene]-1H-1,2,3-triazolo[4,5-b]pyridinium 3-oxide hexafluorophosphate
HOBt: hydroxybenzotriazole
HPLC: high-performance liquid chromatography
IBX: 2-iodoxybenzoic acid
LCMS: liquid chromatography mass spectrometry
MeOH: methanol
MeTHF: methyltetrahydrofuran
min: minute(s)
MsCl: mesyl chloride
MSD: mass selective detector
MTBE: methyl tert-butyl ether
NBS: N-bromosuccinimide
NFSI: N-fluorodi(benzenesulfonyl)amine
Pd(dppf)Cl₂: [1,1′-bis(diphenylphosphino)ferrocene]palladium(II) dichloride
Pd(DTBPF)Cl₂: [1,1′-bis(di-tert-butylphosphino)ferrocene]dichloropalladium(II)
PdCl₂(dppf)CH₂Cl₂ or Pd(dppf)Cl₂·DCM: [1,1′-bis(diphenylphosphino)ferrocene] dichloropalladium(II), complex with dichloromethane
Pet ether: petroleum ether
PS: penicillin-streptomycin
PPTS: pyridinium p-toluenesulfonate
prep: preparative
quant.: quantitative
RT or r.t.: room temperature
rt: retention time
sat.: saturated
SFC: supercritical-fluid chromatography
TBAF: tetrabutylammonium fluoride
TBDPS: tert-butyldiphenylsilyl
TBDPS: tert-butyldiphenylsilyl
TBM: target binding moiety
TEA: triethylamine
TFA: trifluoroacetic acid
TFAA: trifluoroacetic anhydride
THF: tetrahydrofuran
THP: tetrahydropyran
TLC: thin layer chromatography
UPLC: ultra-performance liquid chromatography

### Synthetic Examples

### Synthesis of the CBM Molecule

The numbering of the intermediate compounds referred to in this section, as well as in other sections, is limited to each section only. For instance, intermediate 3 in General Procedure CBM-1 and intermediate 3 in General Procedure CBM-2 are described in different sections and, as such, refer to different compounds.

### General Procedure CBM-1

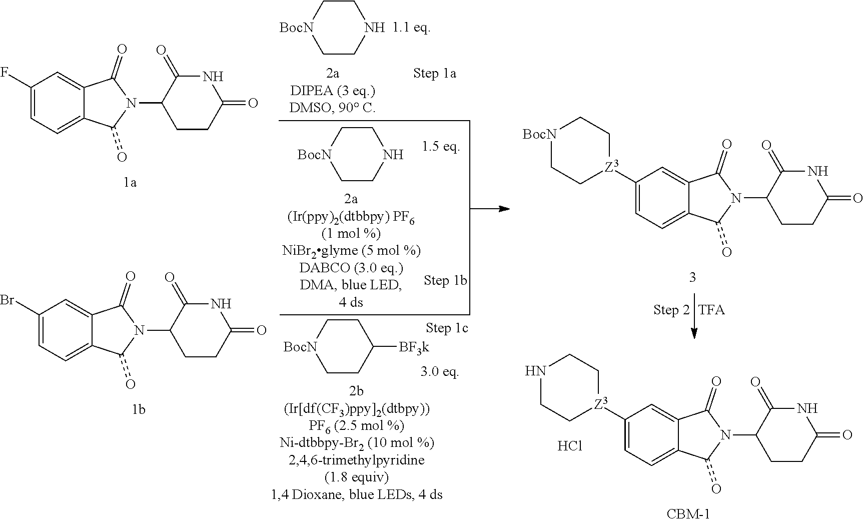

wherein Z³ is N or CH.

Step 1a: A sealed tube was charged with 1a, tert-butyl piperazine-1-carboxylate 2a (1.1 equiv), DIPEA (3.0 equiv) and DMSO (15 mL). The tube was sealed and heated at 90° C. for 72 h. The progress of reaction was followed by HPLC. Upon completion the reaction mixture was directly purified by reversed phase chromatography. The pure fractions were combined, and concentrated under reduced pressure to afford product.

Step 1b: To an oven-dried 40 mL vial equipped with a magnetic stir bar under ambient atmosphere, 3-(6-bromo-1-oxo-isoindolin-2-yl)piperidine-2,6-dione 1b (500 mg, 1.55 mmol) tert-butyl piperazine-1-carboxylate 2a (1.5 equiv), DABCO (3.0 equiv) and (Ir(ppy)2(dtbbpy)PF₆ (1 mol %) were added, followed by DMA (10 mL). Then dibromonickel; 1,2-dimethoxyethane (5 mol %) was added as a solution in DMA (0.5 mL) under N₂. The reaction was placed in front of one 30 W blue LED at 25° C. for 96 h. Material was added into water dropwise, the formed solid was filtered and concentrated. The residue was purified by prep-HPLC to give product.

Step 1c: To a solution of 3-(6-bromo-1-oxo-isoindolin-2-yl)piperidine-2,6-dione 1b, potassium;(1-tert-butoxycarbonyl-4-piperidyl)-trifluoro-boranuide 2b (3.0 equiv) and 2,4,6-Trimethylpyridine (1.8 equiv) in 1,4-Dioxane (0.15 M) in a Schlenk flask was added a solution of (Ir[dF(CF₃)ppy]2(dtbpy))PF₆ (2 mol %) in 1,4-Dioxane (1 mL). A solution of Ni-dtbbpy-Br₂ (10 mol %) in 1,4-Dioxane (1 mL), which had been sonicated for 1 minute, was then added. The reaction mixture was degassed 3× using the freeze-pump-thaw method. The flask was then placed under a dry nitrogen atmosphere and sealed with a parafilm. The reaction mixture was stirred under blue LED irradiation for 4 days. The solution was evaporated until it was dry before dissolving in DMSO for purification by reverse phase chromatography. The pure fractions were combined to afford product.

Step 2: TFA (20 equiv) was added to a solution of 3 in CH₂Cl₂ (0.2 M) and the mixture was stirred at room temperature for 2 h. Upon completion by HPLC, the volatiles removed in vacuum and the residue co-evaporated with MeCN (3×) and MTBE (2×). The residue was purified by reverse phase chromatography. The pure fractions were concentrated under reduced pressure to afford product CBM-1 as a TFA salt.

### Example S1. 2-(2,6-dioxo-3-piperidyl)-5-piperazin-1-yl-isoindoline-1,3-dione trifluoroacetic acid salt (C-2)

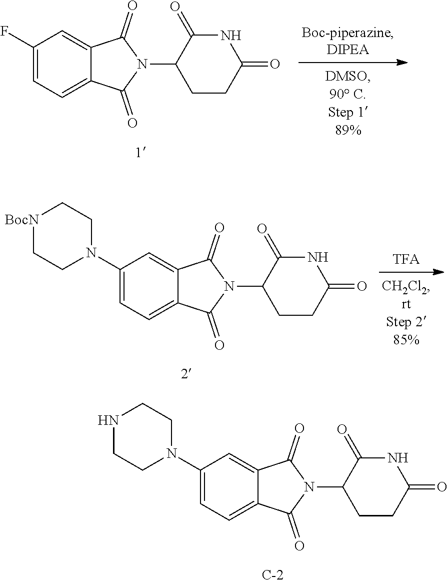

Step 1′. Preparation of tert-Butyl 4-[2-(2,6-dioxo-3-piperidyl)-1,3-dioxo-isoindolin-5-yl]piperazine-1-carboxylate (2′): A sealed tube was charged with 2-(2,6-dioxo-3-piperidyl)-5-fluoro-isoindoline-1,3-dione 1′ (2.0 g, 7.24 mmol), tert-butyl piperazine-1-carboxylate (1.4 g, 7.52 mmol), DIPEA (3.87 mL, 22.2 mmol) and DMSO (15 mL). The tube was sealed and heated at 90° C. for 72 h. The progress of reaction was followed by HPLC. Upon completion the reaction mixture was directly purified by reversed phase chromatography C18 RediSep Rf Gold with 5-95% MeOH/0.1% formic acid in H₂O as eluent. The pure fractions were combined, and concentrated under reduced pressure to afford 2′ (2.86 g, 89% yield) as a yellow solid.

LCMS method 1: 99.9% purity at 215 nm, [M−tBu+H]⁺=387.2 m/z, [M+Na]⁺=466.2 m/z.

¹H NMR (400 MHz, DMSO-d6) δ ppm 1.42 (s, 9H), 1.99-2.09 (m, 1H), 2.52-2.64 (m, 2H), 2.82-2.95 (m, 1H), 3.47 (m, 8H), 5.07 (dd, J=13.0, 5.4 Hz, 1H), 7.24 (dd, J=8.6, 2.2 Hz, 1H), 7.35 (d, J=2.0 Hz, 1H), 7.70 (d, J=8.3 Hz, 1H), 11.08 (s, 1H).

Step 2′. Preparation of 2-(2,6-dioxo-3-piperidyl)-5-piperazin-1-yl-isoindoline-1,3-dione trifluoroacetic acid salt (C-2): TFA (10.55 mL, 129.5 mmol) was added to a solution of tert-butyl 4-[2-(2,6-dioxo-3-piperidyl)-1,3-dioxo-isoindolin-5-yl]piperazine-1-carboxylate 2′ (2.87 g, 6.48 mmol) in CH₂Cl₂ (30 mL) and the mixture was stirred at room temperature for 2 h. Upon completion by HPLC, the volatiles removed in vacuum and the residue co-evaporated with MeCN (3×) and MTBE (2×). The residue was purified by C18 RediSep Rf Gold reverse phase chromatography with 5-20% MeCN/0.05% TFA in H₂O as eluent. The pure fractions were concentrated under reduced pressure to afford C-2 (2.52 g, 85% yield) as a yellow solid as a TFA salt.

LCMS method 1: 99.9% purity at 215 nm, [M+H]⁺=343.2 m/z.

¹H NMR (400 MHz, DMSO-d6) δ ppm 1.95-2.10 (m, 1H), 2.52-2.64 (m, 2H), 2.82-2.97 (m, 1H), 3.18-3.31 (m, 4H), 3.62-3.73 (m, 4H), 5.09 (dd, J=12.8, 5.5 Hz, 1H), 7.33 (dd, J=8.8, 2.2 Hz, 1H), 7.46 (d, J=2.0 Hz, 1H), 7.76 (d, J=8.3 Hz, 1H), 11.09 (s, 1H).

### Example S2. 3-(1-oxo-6-piperazin-1-yl-isoindolin-2-yl)piperidine-2,6-dione (C-7)

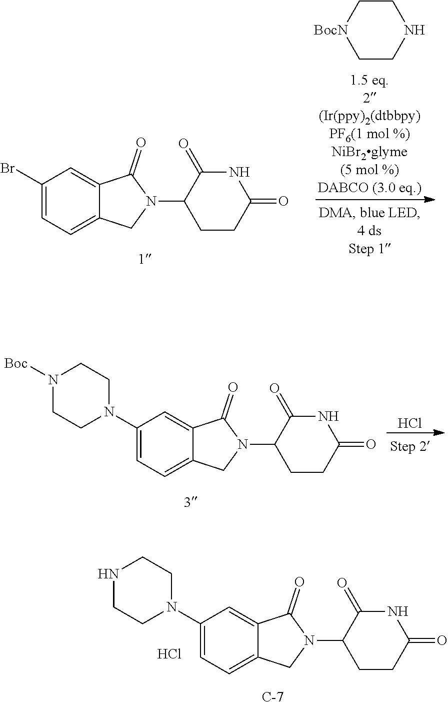

Step 1″. Preparation of tert-butyl 4-[2-(2,6-dioxo-3-piperidyl)-3-oxo-isoindolin-5-yl]piperazine-1-carboxylate (3″): To an oven-dried 40 mL vial equipped with a magnetic stir bar under ambient atmosphere, 3-(6-bromo-1-oxo-isoindolin-2-yl)piperidine-2,6-dione 1″ (500 mg, 1.55 mmol) tert-butyl piperazine-1-carboxylate 2″ (432 mg, 2.32 mmol), DABCO (521 mg, 4.64 mmol) and (Ir(ppy)2(dtbbpy)PF₆ (14.2 mg, 15.47 umol) were added, followed by DMA (10 mL). Then dibromonickel; 1,2-dimethoxyethane (23.9 mg, 77.4 umol) was added as a solution in DMA (0.5 mL) under N₂. The reaction was placed 6 cm in front of one 30 W blue LED at 25° C. for 96 h. LCMS showed the major was product. The combined 8 batches were added into water (200 mL) dropwise, the formed solid was filtered and concentrated. The residue was purified by prep-HPLC (column: Phenomenex luna c18 250 mm x100 mm×10 um; mobile phase: [water (0.1% TFA)-ACN]; B %: 16%-46%, 25 min) to give tert-butyl 4-[2-(2,6-dioxo-3-piperidyl)-3-oxo-isoindolin-5-yl]piperazine-1-carboxylate 3″ (2.5 g, 47.1% yield) as a gray solid.

¹H NMR (400 MHz, DMSO-d6) δ: 10.97 (s, 1H), 7.45 (d, J=8.8 Hz, 1H), 7.30-7.27 (m, 1H), 7.20 (d, J=2.0 Hz, 1H), 5.13-5.08 (m, 1H), 4.30 (dd, J=16.8 Hz, 51.6 Hz, 2H), 3.48 (d, J=4.8 Hz, 4H), 3.16 (d, J=5.2 Hz, 4H), 2.92-2.68 (m, 1H), 2.62-2.51 (m, 1H), 2.41-2.37 (m, 1H), 2.01-1.99 (m, 1H), 1.45 (s, 9H).

Step 2″. 3-(1-oxo-6-piperazin-1-yl-isoindolin-2-yl)piperidine-2,6-dione (C-7): tert-butyl 4-[2-(2,6-dioxo-3-piperidyl)-3-oxo-isoindolin-5-yl]piperazine-1-carboxylate 3″ (5 g, 11.7 mmol) was added to HCl (12 N, 15 mL) at 0° C. The reaction mixture was stirred at 20° C. for 1 h. LCMS showed the most was product. The reaction mixture was diluted with MeCN (500 mL) at 0-10° C., the formed solid was filtered and the cake was dried to give product 12 g. Base on this yield, 5 g of starting material to give 3-(1-oxo-6-piperazin-1-yl-isoindolin-2-yl)piperidine-2,6-dione C-7 (4.3 g, HCl salt, yield 100%) as a gray solid.

¹H NMR (400 MHz, DMSO-d6) δ: 10.98 (s, 1H), 9.34 (s, 2H), 7.49 (d, J=5.2 Hz, 1H), 7.33-7.27 (m, 2H), 5.13-5.09 (m, 1H), 4.30 (dd, J=17.2 Hz, J=58.2 Hz, 2H), 3.46 (d, J=4.8 Hz, 4H), 3.23 (d, J=4.8 Hz, 1H), 2.93-2.78 (m, 1H), 2.62-2.51 (m, 1H), 2.48-2.37 (m, 1H), 2.01-1.98 (m, 1H).

CBM intermediates prepared using General Procedure CBM-1 are summarized in Table 2.

TABLE 2
CBM Intermediates Prepared via General Procedure CBM-1

| | Structure | Heterobicycle | Amine |
|---|---|---|---|
| C-2 | 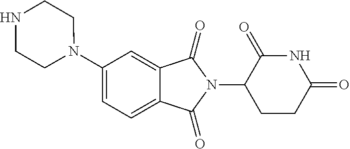 [SMILES: O=C1CCC(N2C(=O)c3ccc(N4CCNCC4)cc3C2=O)C(=O)N1] | 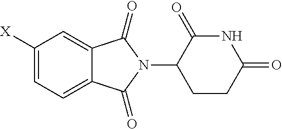 | 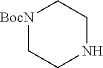 [SMILES: CC(C)(C)OC(=O)N1CCNCC1] |
| C-3 | 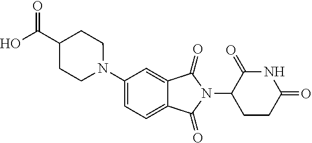 [SMILES: O=C1CCC(N2C(=O)c3ccc(N4CCC(C(=O)O)CC4)cc3C2=O)C(=O)N1] | 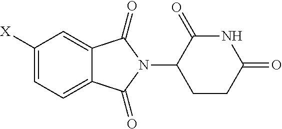 | 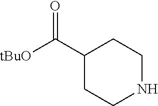 [SMILES: CC(C)(C)OC(=O)C1CCNCC1] |
| | TFA deprotection of tBu ester | | |
| C-5 | 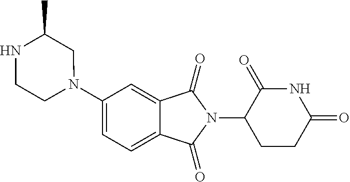 [SMILES: C[C@H]1CN(c2ccc3c(c2)C(=O)N(C2CCC(=O)NC2=O)C3=O)CCN1] | 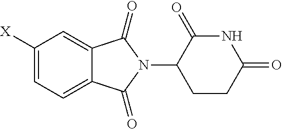 | 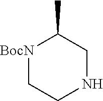 |
| C-7 | 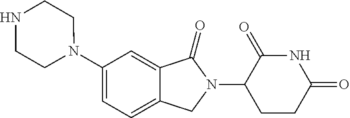 [SMILES: O=C1CCC(N2Cc3ccc(N4CCNCC4)cc3C2=O)C(=O)N1] | 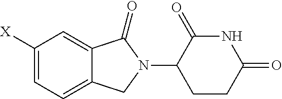 | 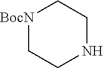 [SMILES: CC(C)(C)OC(=O)N1CCNCC1] |
| C-8 | 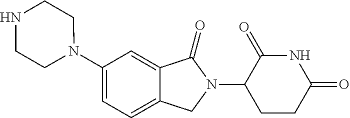 [SMILES: O=C1CCC(N2Cc3ccc(N4CCNCC4)cc3C2=O)C(=O)N1] | 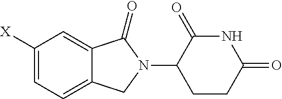 | 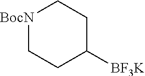 |
| | Use step 1C conditions | | |
| C-9 | 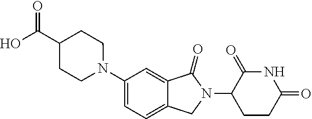 [SMILES: O=C1CCC(N2Cc3ccc(N4CCC(C(=O)O)CC4)cc3C2=O)C(=O)N1] | 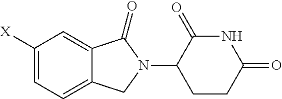 | 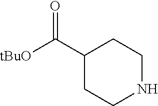 [SMILES: CC(C)(C)OC(=O)C1CCNCC1] |
| | TFA deprotection of tBu ester | | |
| C-11 | 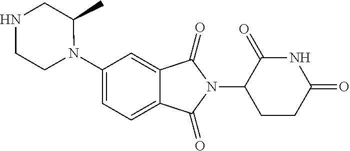 [SMILES: C[C@@H]1CNCCN1c1ccc2c(c1)C(=O)N(C1CCC(=O)NC1=O)C2=O] | 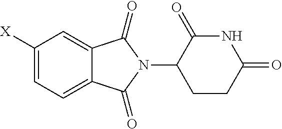 | 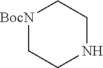 [SMILES: CC(C)(C)OC(=O)N1CCNCC1] |
| C-13 | 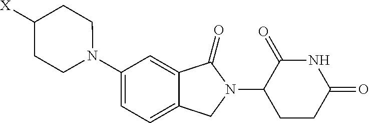 | 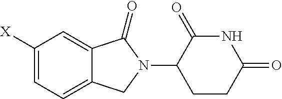 | 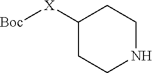 |
| | X = NH₂ or NHMe | | |
| C-16 | 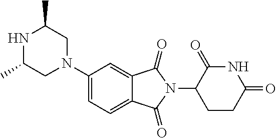 [SMILES: C[C@H]1CN(c2ccc3c(c2)C(=O)N(C2CCC(=O)NC2=O)C3=O)C[C@H](C)N1] | 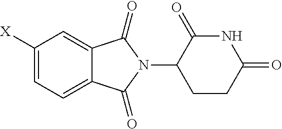 | 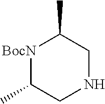 |
| C-18 | 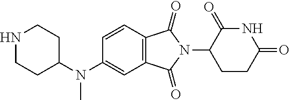 [SMILES: CN(c1ccc2c(c1)C(=O)N(C1CCC(=O)NC1=O)C2=O)C1CCNCC1] | 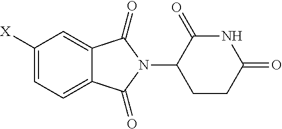 | 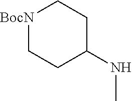 |
| C-47 | 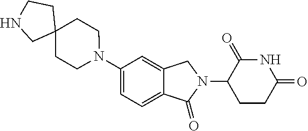 [SMILES: O=C1CCC(N2Cc3cc(N4CCC5(CCNC5)CC4)ccc3C2=O)C(=O)N1] | 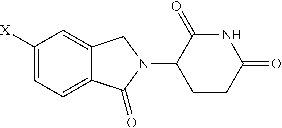 | 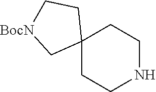 [SMILES: CC(C)(C)OC(=O)N1CCC2(CCNCC2)C1] |

### General Procedure CBM-2

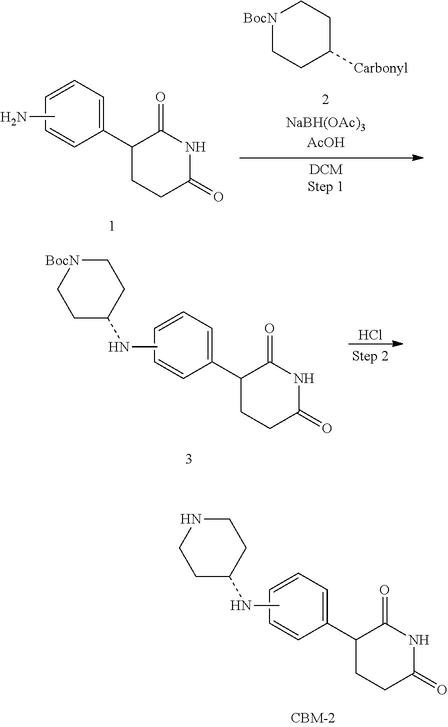

Step 1: To a solution of amine 1 (1 eq.) and carbonyl 2 (1.2 eq.) in DCM (0.24 M) was added NaBH(OAc)₃ (1.2 eq.) and AcOH (1 eq.). The mixture was stirred at room temperature. After LCMS showed complete conversion, the reaction mixture was quenched with water, extracted, dried, filtrated and evaporated under reduced pressure. The crude mixture was then purified by normal phase flash chromatography, affording 3.

Step 2: To 3 (1 eq.) in THF (0.1 M) was added 4 M HCl in dioxane (15 eq.). The reaction mixture was stirred at rt. After 1 h, LCMS showed complete conversion, the solvent was removed under reduced pressure and the residue was co-evaporated with MeCN (3×). The residue was dried under high vacuum to give CBM-2.

### Example S3. 3-[4-(4-piperidylmethylamino)phenyl]piperidine-2,6-dione (C-6)

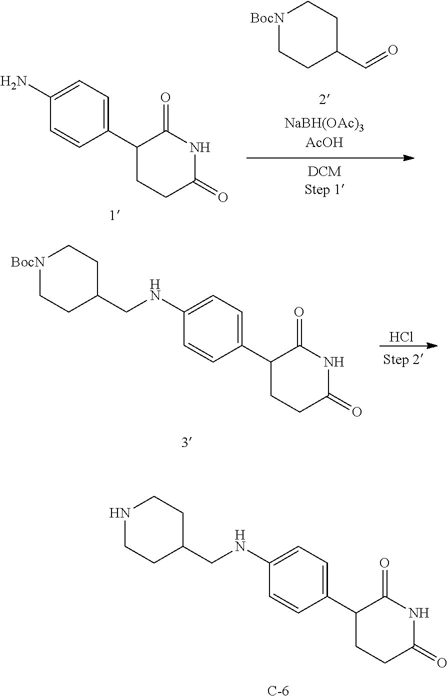

Step 1′. Preparation of tert-butyl N-[4-[[1-[4-(2,6-dioxo-3-piperidyl)phenyl]-4 piperidyl]oxymethyl]cyclohexyl]carbamate (3): To a solution of 3-(4-aminophenyl)piperidine-2,6-dione 1′ (250 mg, 1.22 mmol, 1 eq.) and tert-butyl 4-formylpiperidine-1-carboxylate 2′ (310 mg, 1.45 mmol, 1.19 eq.) in DCM (5 mL, 0.24 M) was added NaBH(OAc)₃ (310 mg, 1.46 mmol, 1.19 eq.) and AcOH (0.07 mL, 1.22 mmol, 1 eq.). The mixture was stirred at room temperature. After an overnight period, LCMS showed complete conversion into compound 3. The reaction mixture was quenched with water, extracted with DCM 3×, dried over sodium sulfate, filtrated and evaporated under reduced pressure. The crude mixture was then purified by normal phase flash chromatography (40 g column, solid deposit, elution 0% to 100% Hept./EtOAc over 15 CV, product came out at 100% EtOAc). Fractions were combined and concentrated, affording 3′ (440 g, 89% yield) as a white solid.

LCMS method 1: 96.8% purity at 215 nm, [M-Boc+H]⁺=302.2.

¹H NMR (400 MHz, DMSO-d6) δ ppm 0.96-1.09 (m, 2H), 1.39 (s, 9H), 1.63-1.77 (m, 3H), 1.94-2.14 (m, 2H), 2.40-2.48 (m, 1H), 2.54-2.76 (m, 3H), 2.88 (br t, J=5.7 Hz, 2H), 3.62 (dd, J=10.5, 5.1 Hz, 1H), 3.94 (br d, J=11.7 Hz, 2H), 5.61 (t, J=5.7 Hz, 1H), 6.51 (d, J=8.6 Hz, 2H), 6.89 (d, J=8.3 Hz, 2H), 10.72 (s, 1H).

Step 2′. Preparation of 3-[4-(4-piperidylmethylamino)phenyl]piperidine-2,6-dione (C-6): To tert-butyl 4-[[4-(2,6-dioxo-3-piperidyl)anilino]methyl]piperidine-1-carboxylate 3′ (419 mg, 1.04 mmol, 1 eq.) in THF (10.4 mL, 0.1 M) was added 4 M HCl in dioxane (3.91 mL, 15.65 mmol, 15 eq.). The reaction mixture was stirred at rt. After 1 h, LCMS showed complete conversion into compound 4. The solvent was removed under reduced pressure and the residue was co-evaporated with MeCN (3×). The residue was dried under high vacuum to give C-6 (353 mg, quantitative yield) as a white solid.

LCMS method 1: 97.8% purity at 215 nm, [M+H]⁺=302.2.

¹H NMR (400 MHz, DMSO-d6) δ ppm 1.39 (q, J=11.8 Hz, 2H), 1.86-2.04 (m, 4H), 2.06-2.21 (m, 1H), 2.40-2.48 (m, 1H), 2.59-2.71 (m, 1H), 2.83 (q, J=11.6 Hz, 2H), 3.04 (br d, J=5.6 Hz, 2H), 3.21-3.31 (m, 2H), 3.68-3.80 (m, 1H), 6.80-7.02 (m, 2H), 7.03-7.14 (m, 2H), 8.59-8.77 (m, 1H), 8.83-8.99 (m, 1H), 10.79 (s, 1H).

CBM intermediates prepared using General Procedure CBM-2 are summarized in Table 3.

TABLE 3
CBM Intermediates Prepared via General Procedure CBM-2.

| | Structure | Heterobicycle | Carbonyl |
|---|---|---|---|
| C-6/22 | 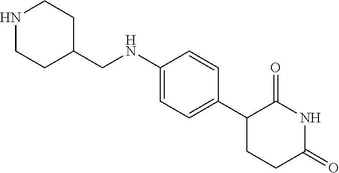 [SMILES: O=C1CCC(c2ccc(NCC3CCNCC3)cc2)C(=O)N1] | 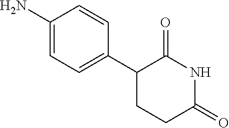 [SMILES: Nc1ccc(C2CCC(=O)NC2=O)cc1] | 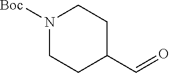 [SMILES: CC(C)(C)OC(=O)N1CCC(C=O)CC1] |
| C-24 | 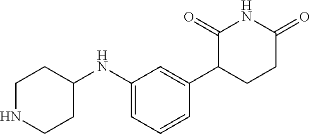 [SMILES: O=C1CCC(c2cccc(NC3CCNCC3)c2)C(=O)N1] | 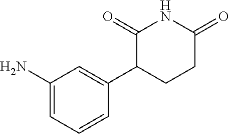 [SMILES: Nc1cccc(C2CCC(=O)NC2=O)c1] | 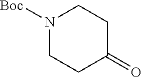 [SMILES: CC(C)(C)OC(=O)N1CCC(=O)CC1] |
| C-27 | 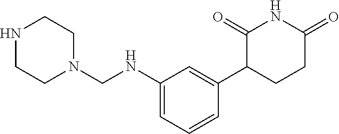 [SMILES: O=C1CCC(c2cccc(NCN3CCNCC3)c2)C(=O)N1] | 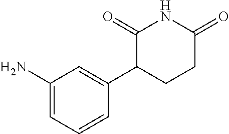 [SMILES: Nc1cccc(C2CCC(=O)NC2=O)c1] | 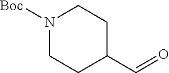 [SMILES: CC(C)(C)OC(=O)N1CCC(C=O)CC1] |

### General Procedure CBM-3

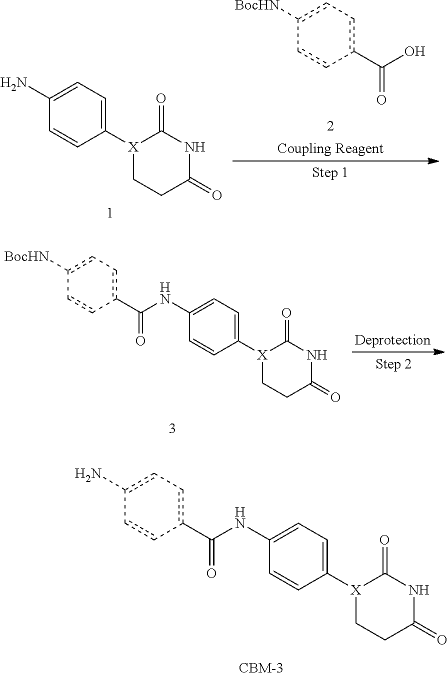

wherein X is N or CR¹³ as defined in Formula (I′) or (I).

Step 1: To a flask was added amine 1 (1 eq.), carboxylic acid 2 (1 eq.), and DIPEA (5 eq.) in DMF (0.12 M) under N₂. The resulting solution was stirred at room temperature. Then, PyAOP (1.3 eq.) or other coupling reagent was added in one portion. The reaction was stirred at room temperature. Upon completion, the reaction mixture was directly loaded for reverse phase purification and clean factions were combined and concentrated to give product 3.

Step 2: To a solution of amide 3 (1 eq.) in DCM (0.13 M) was added TFA (15 eq.) or other deprotection reagent. The reaction mixture was stirred at room temperature. Upon completion, the reaction mixture was concentrated under reduced pressure and the residue was co-evaporated with THF and toluene to give CBM-3.

### Example S4. 4-amino-N-[4-(2,6-dioxo-3-piperidyl)phenyl]benzamide (C-10)

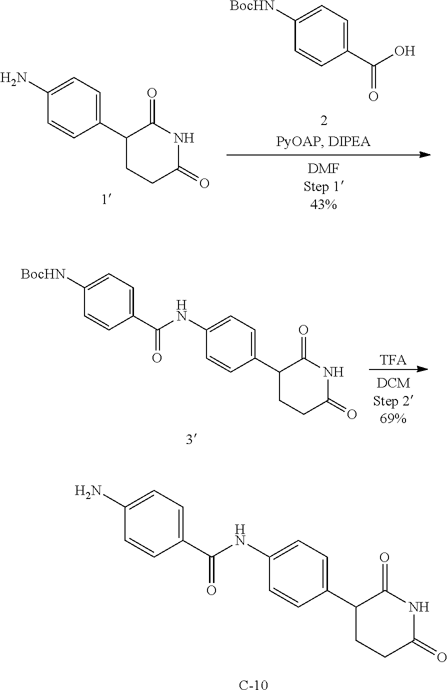

Step 1′. Preparation of tert-butyl N-[4-[[4-(2,6-dioxo-3-piperidyl)phenyl]carbamoyl]phenyl]carbamate (3′): To a round bottom flask was added 3-(4-aminophenyl)piperidine-2,6-dione 1′ (300 mg, 1.47 mmol, 1 eq.), 4-(tert-butoxycarbonylamino)benzoic acid 2′ (348.5 mg, 1.47 mmol, 1 eq.), and DIPEA (1.28 mL, 7.34 mmol, 5 eq.) in DMF (12.2 mL, 0.12 M) under N₂. The resulting solution was stirred at room temperature for 10 minutes. Then, PyAOP (995.65 mg, 1.91 mmol, 1.3 eq.) was added in one portion. The reaction was stirred at room temperature. After 16 h, LCMS showed complete conversion into compound 2. The reaction mixture was directly loaded for reverse phase FC purification (50 g C18 column, liquid deposit (reaction mixture), 5% MeCN/0.1% HCOOH over 3 CV, then 5 to 40% MeCN/0.1% HCOOH over 2 CV, then 40 to 100% MeCN/0.1% HCOOH over 15 CV, product came out at 60% MeCN). Fractions were combined and concentrated to give 3′ (267 mg, 43% yield) as a tan solid.

LCMS method 1: 99.9% purity at 215 nm, [M+H]⁺=424.2.

¹H NMR (400 MHz, DMSO-d6) δ ppm 1.49 (s, 9H), 2.00-2.10 (m, 1H), 2.13-2.26 (m, 1H), 2.45-2.48 (m, 1H), 2.61-2.73 (m, 1H), 3.82 (dd, J=11.2, 4.6 Hz, 1H), 7.19 (d, J=8.6 Hz, 2H), 7.58 (d, J=8.8 Hz, 2H), 7.70 (d, J=8.6 Hz, 2H), 7.89 (d, J=8.8 Hz, 2H), 9.68 (s, 1H), 10.07 (s, 1H), 10.82 (s, 1H).

Step 2′. Preparation of 4-amino-N-[4-(2,6-dioxo-3-piperidyl)phenyl]benzamide (C-10): To a solution of tert-butyl N-[4-[[4-(2,6-dioxo-3-piperidyl)phenyl]carbamoyl]phenyl]carbamate 3′ (267 mg, 0.63 mmol, 1 eq.) in DCM (5 mL, M) was added TFA (1.03 mL, 12.61 mmol, 15 eq.). The reaction mixture was stirred at room temperature. After 30 minutes, LCMS showed full conversion into compound 4. The reaction mixture was concentrated under reduced pressure and the residue was co-evaporated with THF (2×) and toluene (2×) to give C-10 (207 mg, 69% yield) as a tan solid as a trifluoroacetic acid salt.

LCMS method 1: 92.3% purity at 215 nm, [M−CF₃COOH+H]⁺=324.2.

¹H NMR (400 MHz, DMSO-d6) δ ppm 2.00-2.06 (m, 1H), 2.13-2.24 (m, 1H), 2.44-2.49 (m, 1H), 2.61-2.72 (m, 1H), 3.81 (dd, J=11.1, 5.0 Hz, 1H), 6.72 (d, J=8.6 Hz, 2H), 7.16 (d, J=8.3 Hz, 2H), 7.69 (d, J=8.6 Hz, 2H), 7.76 (d, J=8.8 Hz, 2H), 9.83 (s, 1H), (s, 1H). Three protons were missing.

### Example S5. N-[4-(2,4-dioxohexahydropyrimidin-1-yl)phenyl]piperidine-4-carboxamide hydrochloride (C-16)

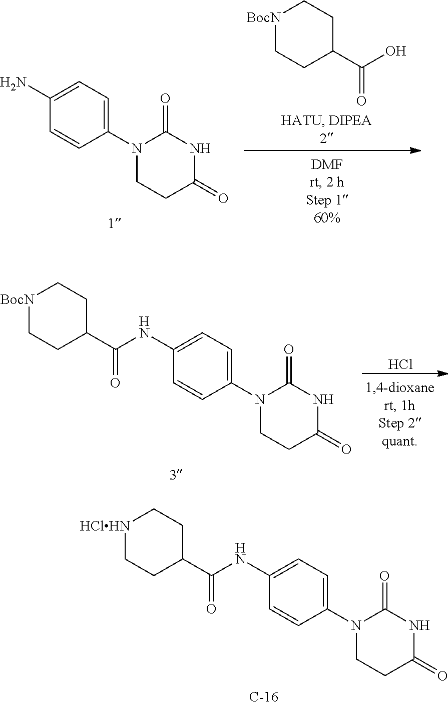

Step 1″. Preparation of tert-butyl 4-[[4-(2,4-dioxohexahydropyrimidin-1-yl)phenyl]carbamoyl]piperidine-1-carboxylate (3″): 1-(4-aminophenyl)hexahydropyrimidine-2,4-dione 1″ (191 mg, 0.931 mmol, 1.1 equiv) and 1-tert-Butoxycarbonylpiperidine-4-carboxylic acid 2″ (194 mg, 0.846 mmol, 1.0 equiv) were dissolved in DMF (4.7 mL), then DIPEA (1.47 mL, 8.46 mmol, 10.0 equiv) and HATU (418 mg, 1.10 mmol, 1.3 equiv) were added in sequence. The reaction mixture was stirred for 2 hours at room temperature. The reaction mixture was injected directly in a column for reversed-phase flash chromatography purification (MeCN in 0.1% HCOOH(aq), 5% (3 CV)→100%, 100 g RediSep Rf Gold® C18Aq, 10 CV, λ=214-254 nm). Evaporation of the fractions yielded 3″ (211 mg, 0.507 mmol, 60% yield) as a yellow solid.

LCMS method 1: 99.9% purity at 215 nm, [M−t−Bu+H]⁺=317.2.

¹H NMR (400 MHz, DMSO-d6) δ ppm 1.39-1.42 (m, 9H), 1.42-1.54 (m, 2H), 1.77 (br d, J=11.5 Hz, 2H), 2.44-2.49 (m, 1H), 2.69 (t, J=6.7 Hz, 2H), 2.72-2.94 (m, 2H), 3.73 (t, J=6.7 Hz, 2H), 3.99 (br d, J=11.7 Hz, 2H), 7.24 (d, J=8.9 Hz, 2H), 7.59 (d, J=8.8 Hz, 2H), 9.96 (s, 1H), 10.32 (s, 1H).

Step 2″. Preparation of N-[4-(2,4-dioxohexahydropyrimidin-1-yl)phenyl]piperidine-4-carboxamide hydrochloride (C-16): HCl 4.0 M in 1,4-dioxane (1.5 mL, 6.00 mmol, 10.0 equiv) was added to tert-butyl 4-[[4-(2,4-dioxohexahydropyrimidin-1-yl)phenyl]carbamoyl]piperidine-1-carboxylate 3″ (250 mg, 0.600 mmol, 1.0 equiv) and the mixture was stirred for one hour at room temperature. The solvent was evaporated under reduced pressure and the residue was then dried under high vacuum to remove all the volatiles. The crude product C-16 (212 mg, 0.600 mmol, quantitative yield) was obtained as a white solid that was used as-is in next step.

LCMS method 1: 99.9% purity at 215 nm, [M−HCl+H]⁺=317.2.

¹H NMR (400 MHz, DMSO-d6) δ ppm 1.72-1.89 (m, 2H), 1.91-2.03 (m, 2H), 2.59-2.66 (m, 1H), 2.69 (t, J=6.7 Hz, 2H), 2.84-3.01 (m, 2H), 3.30-3.33 (m, 2H), 3.74 (t, J=6.7 Hz, 2H), 7.25 (d, J=8.8 Hz, 2H), 7.59 (d, J=8.8 Hz, 2H), 8.41 (br s, 1H), 8.70 (br s, 1H), 10.11 (s, 1H), 10.33 (s, 1H).

CBM intermediates prepared using General Procedure CBM-3 are summarized in Table 4.

TABLE 4
CBM Intermediates Prepared via General Procedure CBM-3

| | Structure | Heterobicycle | Acid |
|---|---|---|---|
| C-10 | 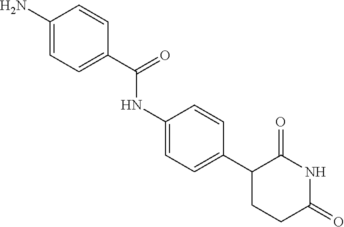 [SMILES: Nc1ccc(C(=O)Nc2ccc(C3CCC(=O)NC3=O)cc2)cc1] | 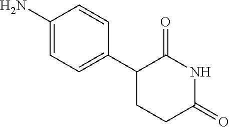 [SMILES: Nc1ccc(C2CCC(=O)NC2=O)cc1] | 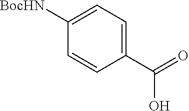 [SMILES: CC(C)(C)OC(=O)Nc1ccc(C(=O)O)cc1] |
| C-15 | 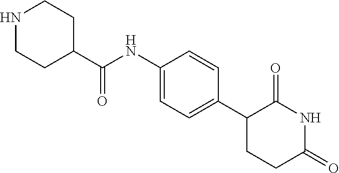 [SMILES: O=C1CCC(c2ccc(NC(=O)C3CCNCC3)cc2)C(=O)N1] | 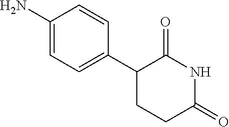 [SMILES: Nc1ccc(C2CCC(=O)NC2=O)cc1] | 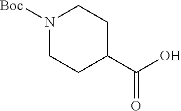 [SMILES: CC(C)(C)OC(=O)N1CCC(C(=O)O)CC1] |
| C-16 | 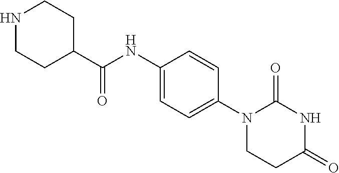 [SMILES: O=C1CCN(c2ccc(NC(=O)C3CCNCC3)cc2)C(=O)N1] | 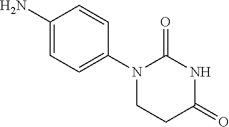 [SMILES: Nc1ccc(N2CCC(=O)NC2=O)cc1] | 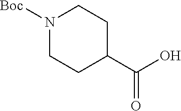 [SMILES: CC(C)(C)OC(=O)N1CCC(C(=O)O)CC1] |
| C-20 | 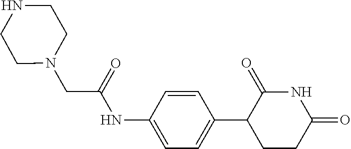 [SMILES: O=C1CCC(c2ccc(NC(=O)CN3CCNCC3)cc2)C(=O)N1] | 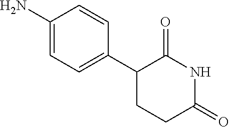 [SMILES: Nc1ccc(C2CCC(=O)NC2=O)cc1] | 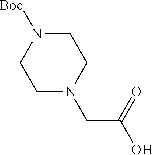 [SMILES: CC(C)(C)OC(=O)N1CCN(CC(=O)O)CC1] |
| C-23 | 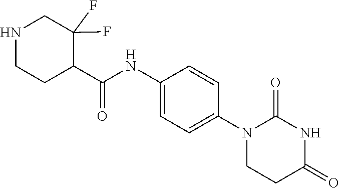 [SMILES: O=C1CCN(c2ccc(NC(=O)C3CCNCC3(F)F)cc2)C(=O)N1] | 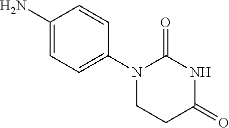 [SMILES: Nc1ccc(N2CCC(=O)NC2=O)cc1] | 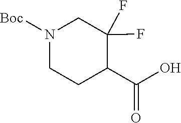 [SMILES: CC(C)(C)OC(=O)N1CCC(C(=O)O)C(F)(F)C1] |
| | Start with dihydrouracil | | |

### General Procedure CBM-4 (CBM-4A and CBM-4B)

### General Procedure CBM-4A

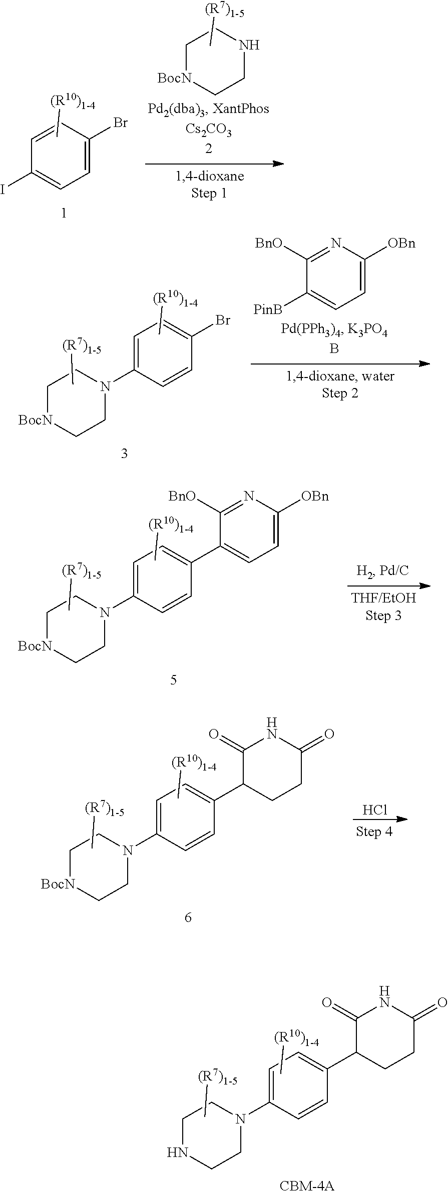

wherein R⁷ and R¹⁰ are as defined in Formula (I′) or (I).

LCMS Method 1. Column: Kinetex XB-C18, 75×3.0 mm, 2.6 Temperature: 45° C., Flow: 1.0 mL/min, run time: 5.0 min. Mobile Phase Conditions: Mobile Phase-A: 5.0 mm Ammonium formate pH 3.3:CH₃CN (98:02), Mobile Phase-B: CH₃CN: Buffer (98:02), Gradient: Initial 98% Mobile Phase A and 2% Mobile Phase B linear gradient to 100% Mobile Phase B for 4.1 min then hold for 0.5 minute. MSD positive.

LCMS Method 2. Column: Kinetex XB-C18, 75×3.0 mm, 2.611m. Temperature: ° C., Flow: 1.0 mL/min, run time: 5.0 min. Mobile Phase Conditions: Mobile Phase-A: 5.0 mm Ammonium formate pH 3.3:CH₃CN (98:02), Mobile Phase-B: CH₃CN: Buffer (98:02), Gradient: Initial 80% Mobile Phase A and 20% Mobile Phase B linear gradient to 100% Mobile Phase B for 4.0 min then hold for 0.5 minute. MSD positive.

LCMS Method 3. Column: Kinetex XB-C18, 50×4.6 mm, 5.011m. Temperature: ° C., Flow: 1.5 mL/min, run time: 6.0 min. Mobile Phase Conditions: Mobile Phase-A: 0.1% TFA in H₂O, Mobile Phase-B: 0.1% TFA in ACN, Gradient: Initial 95% Mobile Phase A and 5% Mobile Phase B linear gradient to 95% Mobile Phase B for 2.5 min then hold for 1.5 minute. MSD positive.

### Step A. Preparation of 2,6-Dibenzyloxy-3-(4,4,5,5-tetramethyl-1,3,2-dioxaborolan-2-yl)pyridine B

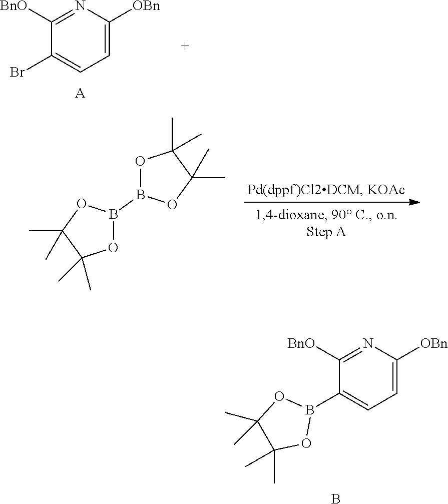

In a sealed tube 2,6-dibenzyloxy-3-bromo-pyridine A (1.0 g, 2.701 mmol, 1.0 eq.), bis(pinacolato)diboron (1.0 g, 4.051 mmol, 1.5 eq.), KOAc (795.2 mg, 8.103 mmol, 3.0 eq.) and Pd(dppf)Cl₂·DCM (220.57 mg, 0.270 mmol, 0.1 eq.) were solubilized in 1,4-dioxane (4.0 mL, M) and nitrogen was bubbled for 10 minutes. The tube sealed and heated at 90° C. for overnight. The reaction mixture was cooled down to room temperature. The reaction was filtered over celite, washed with MeTHF and the filtrate was evaporated. The residue was purified by normal phase flash chromatography (Isco, 24 g, elution was done from 0% EtOAc in heptane to 10% EtOAc over 18 CVs) to give 900 mg of B contaminated by bis(pinacolato). A second purification by normal phases flash chromatography was performed (80 g SNAP column; elution: 0% EtOAc in heptane for 2 CVs, then 0 to 4.5% EtOAc in heptane over 15 CVs, product exited at 4.1% EtOAc) to give 2,6-dibenzyloxy-3-(4,4,5,5-tetramethyl-1,3,2-dioxaborolan-2-yl)pyridine B (210 mg, 19% yield) as a white solid.

LCMS method 1: retention time: 1.915 min, [M−Pin+H]⁺=336.2 and 2.290 min, [M+H]⁺=418.2; 99.9% purity at 215 nm.

¹H NMR (400 MHz, DMSO-d6) δ ppm 1.28 (s, 12H), 5.38 (d, J=5.9 Hz, 4H), 6.42 (d, J=7.8 Hz, 1H), 7.24-7.40 (m, 6H), 7.40-7.44 (m, 2H), 7.53 (d, J=7.1 Hz, 2H), 7.84 (d, J=7.8 Hz, 1H).

Step 1: To a flame-dried sealed tube were added Cs₂CO₃ (2.0 eq.), heterocyclie 2 (1.1 eq.), bromo-iodo 1 (1.0 eq.), XantPhos (0.2 eq.) and Pd₂(dba)₃ (0.1 eq.) were solubilized in dry 1,4-dioxane (0.2 M). The mixture was degassed by sparging with nitrogen for 10 minutes. The tube was sealed and the mixture was stirred at 110° C. Upon complete conversion of the starting materials, the reaction mixture was cooled down to room temperature. Ethyl acetate and water were added, phases were separated, the aqueous phase was extracted 3 times with ethyl acetate, then the combined organic phases were washed once with brine, dried over sodium sulfate, filtered and concentrated under reduced pressure. The residue was purified by normal phase flash chromatography before fractions were combined and concentrated to give bromide product 3.

Step 2: A sealed tube was charged with K₃PO₄ (2.3 eq.), bromide 3 (1.0 eq.), 2,6-dibenzyloxy-3-(4,4,5,5-tetramethyl-1,3,2-dioxaborolan-2-yl)pyridine B (1.2 eq.) and Pd(PPh₃)₄ (0.1 eq.). The mixture was degassed by sparging with nitrogen for 10 minutes. A solution of 1,4-dioxane (0.08 M) and water (0.08 M) was added, then the mixture was degassed by sparging with nitrogen for 10 minutes. The vial was sealed and the reaction was heated overnight at 90° C. Upon complete conversion of the starting materials, the mixture was cooled down to room temperature, the reaction was filtered over celite, washed with EtOAc and the filtrate was evaporated. The residue was purified by normal phase flash chromatography before fractions were combined and concentrated to give pyridine 5.

Step 3: A solution of pyridine 5 (1.0 eq.) in THF/EtOH (0.05 M) was degassed for 15 minutes, then Pd(OH)₂ (0.2 eq.) was degassed by sparging with nitrogen for 5 minutes. Hydrogen was then bubbled in the reaction mixture for 5 minutes and the mixture was stirred in a hot water bath at 50° C. under a hydrogen atmosphere. The mixture was cooled down to room temperature. Ethyl acetate and water were added and phases were separated, the aqueous phase was extracted 3 times with ethyl acetate, then the combined organic phases were dried over sodium sulfate, filtered and concentrated under reduced pressure. The residue was purified by normal phase flash chromatography before fractions were combined and concentrated to give glutarimide 6.

Step 4: To a solution of glutarimide 6 (1.0 eq.) was added HCl 4 M in dioxane (54 eq.). The reaction was stirred at room temperature. After an overnight period, LCMS showed complete conversion into product. The reaction mixture was concentrated under reduced pressure and co-evaporated 3 times with acetonitrile to give product CBM-4A that was used as is directly for the next step.

### General Procedure CBM-4B

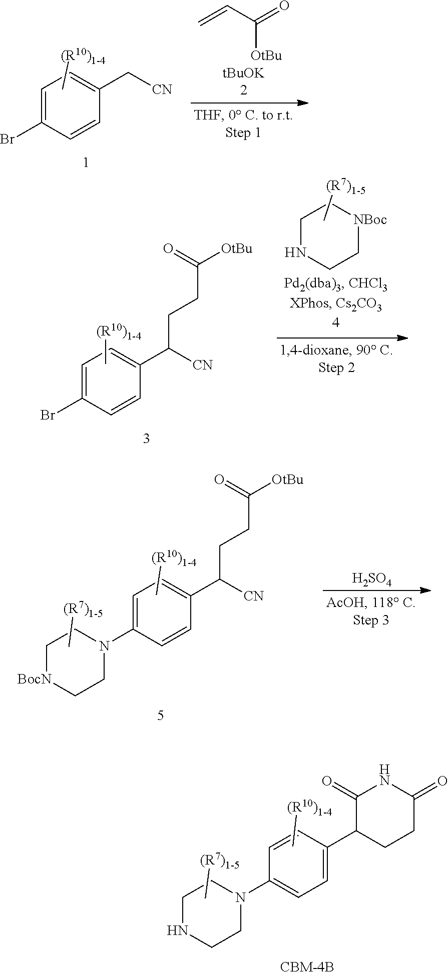

wherein R⁷ and R¹⁰ are as defined in Formula (I′) or (I).

Step 1: A solution of bromide 1 (1.20 g, 6.09 mmol, 1 eq.) and tert-butyl prop-2-enoate 2 (1.05 eq.) in anhydrous THF (0.5 M) was cooled down to 0° C. under nitrogen. tBuOK (0.1 eq.) was added and the reaction was stirred at 0° C. and allowed to slowly warm to room temperature. Upon completion, the reaction mixture was filtered over a pad of Celite, rinsed with EtOAc and the filtrate was concentrated to dryness. The residue was purified by reverse phase flash chromatography and fractions were combined and partially concentrated to dryness. The suspension was then partitioned between saturated aqueous NaHCO₃ and EtOAc. The layers were separated and the aqueous one was back extracted with EtOAc. The combined organics were dried over Na₂SO₄, filtered and concentrated to dryness to give 3.

Step 2: To a sealed tube were added bromide 3 (1 eq.), heterocycle 4 (1.2 eq.), Cs₂CO₃ (2 eq.), XPhos (0.2 eq.) and 1,4-dioxane (0.1 M). The reaction mixture was sparged with nitrogen for 10 min and Pd₂(dba)₃·CHCl₃ (0.1 eq.) was quickly added. The reaction mixture was sparged with nitrogen for a further 10 min and it was then stirred at 90° C. Upon completion, the reaction mixture was filtered over a pad of Celite, rinsed with EtOAc and the filtrate was concentrated to dryness. The residue was purified by reverse phase flash chromatography and rractions were combined and partially concentrated to dryness. The suspension was then partitioned between saturated aqueous NaHCO₃ and EtOAc. The layers were separated and the aqueous one was back extracted with EtOAc. The combined organics were dried over Na₂SO₄, filtered and concentrated to dryness to give 5.

Step 3: To a sealed tube were added 5 (1 eq.), acetic acid (0.2 M) and concentrated sulfuric acid (4 eq.). The reaction mixture was stirred at 118° C. Upon completion, volatiles were removed under reduce pressure and the residue was purified by reverse phase flash chromatography. Fractions were combined and concentrated to give CBM-4B that was used as-is for the next step.

### Example S6. 3-(4-(piperazin-1-yl)phenyl)piperidine-2,6-dione (C-12)

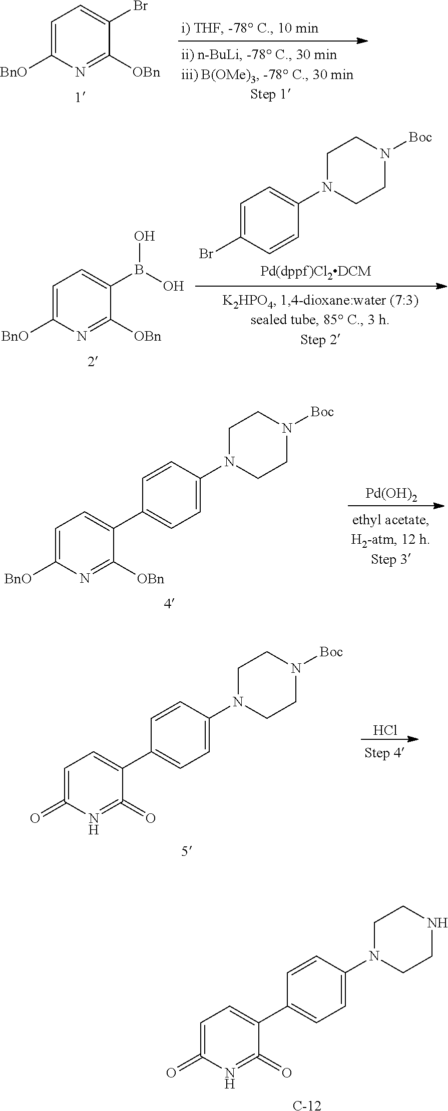

Step 1′. Preparation of (2,6-bis(benzyloxy)pyridin-3-yl)boronic acid 2′: To a stirred solution of 2,6-bis(benzyloxy)-3-bromopyridine 1′ (30.0 g, 81 mmol, 1.0 eq.) in THF (300 mL) at −78° C. was added n-BuLi (48.6 mL, 122 mmol, 1.5 eq.) and stirred at the same temperature for 30 min. Then, trimethyl borate (10.86 mL, 97 mmol, 1.2 eq.) was added dropwise and stirring was continued at the same temperature for another 30 min. The reaction was quenched with sat. aq. ammonium chloride (300 mL) and acidified using 1.5 N HCl solution to maintain the pH 3-4. The reaction mixture was extracted with ethyl acetate (3×500 mL), dried over sodium sulphate, concentrated under reduced pressure to give the crude product. The crude product was triturated with petroleum ether (1000 mL) to afford (2,6-bis(benzyloxy)pyridin-3-yl)boronic acid 2′ (16 g, 50.1% yield) as an off-white solid.

LCMS method 1: retention time: 3.003 min, 85.56% purity at 220 nm, [M+H]⁺=336.0.

¹H NMR (400 MHz, DMSO-d6): δ ppm 5.35 (s, 2H), 5.42 (s, 2H), 6.45 (d, J=8.0 Hz, 1H), 7.30-7.47 (m, 10H), 7.60 (s, 2H), 7.90 (d, J=7.6 Hz, 1H)

Step 2′. Preparation of tert-butyl 4-(4-(2,6-bis(benzyloxy)pyridin-3-yl)phenyl)piperazine-1-carboxylate 4′: To a stirred solution of tert-butyl 4-(4-bromophenyl)piperazine-1-carboxylate 3′ (20 g, 58.6 mmol, 1.0 eq.) in 200 mL of 1,4-dioxane:water (7:3) was added (2,6-bis(benzyloxy)pyridin-3-yl)boronic acid 2′ (23.57 g, 70.3 mmol, 1.2 eq.) followed by potassium phosphate dibasic (20.42 g, 117 mmol, 2.0 eq.). The resulting reaction mixture was degassed with Na for 15 min. and PdCl₂(dppf)·CH₂Cl₂ (4.79 g, 5.86 mmol, 0.1 eq.) was added into the reaction mixture. Then, the reaction mixture was heated at 85° C. in a sealed tube for 3 h. The reaction mixture was cooled to RT, poured into ice-cold water, and extracted with ethyl acetate (2×25 mL). The combined organic extracts were dried over sodium sulfate and concentrated under reduced pressure to obtain the crude product. The crude product was purified by column chromatography using silica gel (230-400 mesh) with 10% ethyl acetate in pet ether to give tert-butyl 4-(4-(2,6-bis(benzyloxy)pyridin-3-yl)phenyl)piperazine-1-carboxylate 4′ (30 g, 85% yield) as an off-white solid.

LCMS method 2: retention time: 2.326 min, 92.15% purity at 220 nm, [M+H]⁺=552.2.

Step 3′. Preparation of tert-butyl 4-(4-(2,6-dioxopiperidin-3-yl)phenyl)piperazine-1-carboxylate 5′: To a stirred solution of tert-butyl 4-(4-(2,6-bis(benzyloxy)pyridin-3-yl)phenyl)piperazine-1-carboxylate 4′ (47 g, 85 mmol, 1.0 eq.) in ethyl acetate (700 mL) was added Pd(OH)₂ (5.98 g, 42.6 mmol, 0.5 eq.). The reaction mixture was stirred at RT for 12 h under H₂ atmosphere. The reaction mixture was filtered through celite bed and the filtrate was concentrated under reduced pressure to obtain tert-butyl 4-(4-(2,6-dioxopiperidin-3-yl)phenyl)piperazine-1-carboxylate 5′ (24 g, 71.7% yield) as an off-white solid.

LCMS method 3: retention time: 1.781 min, 95.26% purity at 220 nm, [M+H]⁺=374.3.

Step 4′. Preparation of 3-(4-(piperazin-1-yl)phenyl)piperidine-2,6-dione C-12. To a stirred solution of tert-butyl 4-(4-(2,6-dioxopiperidin-3-yl)phenyl)piperazine-1-carboxylate (2.0 g, 4.17 mmol, 1 eq.) in DCM (20 mL, 0.208 M) at 0° C. was added dry HCl in 1,4-dioxane (10.42 mL, 41.7 mmol, 4.0 M in dioxane). The reaction mixture was warmed up to RT and stirred for 2 h. It was concentrated under reduced pressure to get crude 3-(4-(piperazin-1-yl)phenyl)piperidine-2,6-dione, 2HCl C-12 (1.9 g) as a brown colour solid. It was used for the next step without further purification.

LCMS method 4: retention time: 0.307 min, [M+H]⁺=274.2.

### Example S7. 3-(2-methoxy-4-piperazin-4-ium-1-yl-phenyl)piperidine-2,6-dione hydrochloride (C-34)

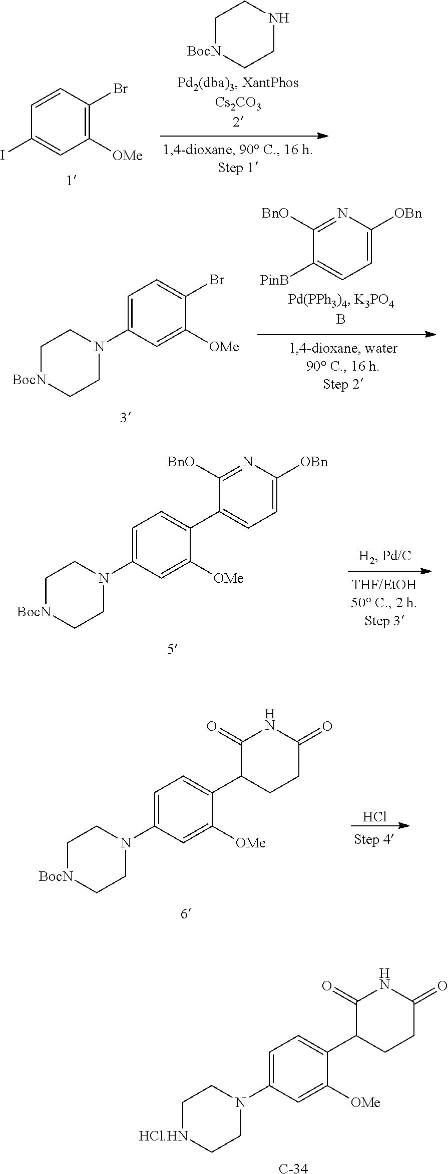

Step 1′. Preparation of tert-butyl 4-(4-bromo-3-methoxy-phenyl)piperazine-1-carboxylate (3′): To a flame-dried sealed tube were added Cs₂CO₃ (2.08 g, 6.39 mmol, 2 eq.), tert-butyl piperazine-1-carboxylate 2′ (654.7 mg, 3.52 mmol, 1.1 eq.), 1-bromo-4-iodo-2-methoxy-benzene 1′ (1 g, 3.2 mmol, 1 eq.), XantPhos (369.81 mg, 0.64 mmol, 0.2 eq.) and Pd₂(dba)₃ (292.63 mg, 0.32 mmol, 0.1 eq.) were solubilized in dry 1,4-dioxane (16 mL, 0.2 M). The mixture was degassed by sparging with nitrogen for 10 minutes. The tube was sealed and the mixture was stirred at 110° C. After an overnight period, LCMS showed complete conversion of the starting materials into compound 3. The reaction mixture was cooled down to room temperature. Ethyl acetate and water were added, phases were separated, the aqueous phase was extracted 3 times with ethyl acetate, then the combined organic phases were washed once with brine, dried over sodium sulfate, filtered and concentrated under reduced pressure. The residue was purified by normal phase flash chromatography (80 g silica column, elution: 0 to 10% MeOH/DCM over 10 CV, then 10% MeOH until the product came out). Fractions were combined and concentrated to give 3′ (742 mg, 62% yield) as a light-yellow solid.

LCMS method 1: 98.5% purity at 215 nm, [M+H]⁺=373.2.

¹H NMR (400 MHz, chloroform-d) δ ppm 1.49 (s, 9H), 3.13 (t, J=5.0 Hz, 4H), 3.59 (t, J=5.0 Hz, 4H), 3.88 (s, 3H), 6.41 (dd, J=8.7, 2.6 Hz, 1H), 6.45-6.51 (m, 1H), 7.38 (d, J=8.6 Hz, 1H).

Step 2′. Preparation of tert-butyl 4-[4-(2,6-dibenzyloxy-3-pyridyl)-3-methoxy-phenyl]piperazine-1-carboxylate (5′): A sealed tube was charged with K₃PO₄ (194.91 mg, mmol, 2.27 eq.), tert-butyl 4-(4-bromo-3-methoxy-phenyl)piperazine-1-carboxylate 3′ (150 mg, 0.40 mmol, 1 eq.), 2,6-dibenzyloxy-3-(4,4,5,5-tetramethyl-1,3,2-dioxaborolan-2-yl)pyridine B (202.32 mg, 0.48 mmol, 1.2 eq.) and Pd(PPh₃)₄ (46.69 mg, 0.04 mmol, 0.1 eq.). The mixture was degassed by sparging with nitrogen for 10 minutes. A solution of 1,4-dioxane (4.04 mL, M) and water (1.01 mL, 0.08 M) was added, then the mixture was degassed by sparging with nitrogen for 10 minutes. The vial was sealed and the reaction was heated overnight at 90° C. After an overnight period, LCMS showed complete conversion of the starting materials into compound 5′. The mixture was cooled down to room temperature, the reaction was filtered over celite, washed with EtOAc and the filtrate was evaporated. The residue was purified by normal phase flash chromatography (40 g silica column, elution: 5 to 50% EtOAc/Heptane over 10 CV, product exited at 25% EtOAc). Fractions were combined and concentrated to give 5′ (195.9 mg, 77% yield) as a yellow solid.

LCMS method 1: 92.0% purity at 215 nm, [M+H]⁺=582.2.

¹H NMR (400 MHz, chloroform-d) δ ppm 1.50 (s, 9H), 3.19 (t, J=5.0 Hz, 4H), 3.61 (t, J=5.2 Hz, 4H), 3.72 (s, 3H), 5.33 (s, 2H), 5.39 (s, 2H), 6.41-6.45 (m, 1H), 6.51-6.57 (m, 2H), 7.17-7.21 (m, 1H), 7.22-7.26 (m, 1H), 7.28-7.39 (m, 7H), 7.41-7.45 (m, 2H), 7.49-7.53 (m, 1H).

Step 3′. Preparation of tert-butyl 4-[4-(2,6-dioxo-3-piperidyl)-3-methoxy-phenyl]piperazine-1-carboxylate (6′): A solution of tert-butyl 4-[4-(2,6-dibenzyloxy-3-pyridyl)-3-methoxy-phenyl]piperazine-1-carboxylate 5′ (140 mg, 0.24 mmol, 1 eq.) in THF/EtOH (0.86 mL:0.86 mL, 0.05 M) was degassed for 15 minutes, then Pd(OH)₂ (33.8 mg, mmol, 0.2 eq.) was degassed by sparging with nitrogen for 5 minutes. Hydrogen was then bubbled in the reaction mixture for 5 minutes and the mixture was stirred in a hot water bath at ° C. under a hydrogen atmosphere for 4 hours. The mixture was cooled down to room temperature. Ethyl acetate and water were added and phases were separated, the aqueous phase was extracted 3 times with ethyl acetate, then the combined organic phases were dried over sodium sulfate, filtered and concentrated under reduced pressure. The residue was purified by normal phase flash chromatography (40 g silica column, elution: 0 to 100% MeOH/DCM over CV). Fractions were combined and concentrated to give 6′ (30 mg, 23% yield) as a light-blue solid.

LCMS method 1: 99.9% purity at 215 nm, [M+H]⁺=404.2.

¹H NMR (400 MHz, chloroform-d) δ ppm 1.49 (s, 9H), 2.03-2.15 (m, 1H), 2.26 (s, 1H), 2.57-2.67 (m, 1H), 2.69-2.78 (m, 1H), 3.14 (t, J=5.2 Hz, 4H), 3.58 (t, J=5.2 Hz, 4H), 3.80 (s, 3H), 6.46-6.52 (m, 2H), 6.97-7.04 (m, 1H), 7.86 (s, 1H).

Step 4′. Preparation of 3-(2-methoxy-4-piperazin-4-ium-1-yl-phenyl)piperidine-2,6-dione hydrochloride (C-34): To a solution of tert-butyl 4-[4-(2,6-dioxo-3-piperidyl)-3-methoxy-phenyl]piperazine-1-carboxylate 6′ (30 mg, 0.07 mmol, 1 eq.) was added HCl 4 M in dioxane (1 mL, 4 mmol, 54 eq.). The reaction was stirred at room temperature. After an overnight period, LCMS showed complete conversion into product. The reaction mixture was concentrated under reduced pressure and co-evaporated 3 times with acetonitrile to give C-34 (35 mg, quantitative yield) as a grey solid that was used as is directly for the next step and assumed that it was in a hydrochloride salt form.

LCMS method 1: 99.9% purity at 215 nm, [M−HCl+H]⁺=304.4.

### Example S8. 3-(2-methoxy-4-piperazin-4-ium-1-yl-phenyl)piperidine-2,6-dione hydrochloride (C-29)

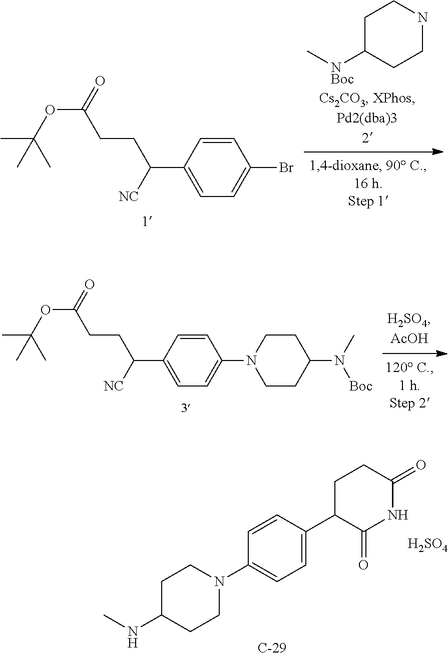

Step 1′. Preparation of Tert-butyl 4-[4-[4-[tert-butoxycarbonyl(methyl)amino]-1-piperidyl]phenyl]-4-cyano-butanoate (3′): In a flame-dried sealed tube, under nitrogen atmosphere were introduced Cs₂CO₃ (1.01 g, 3.084 mmol, 2.0 eq.), tert-butyl N-methyl-N-(4-piperidyl)carbamate 2′ (396.6 mg, 1.850 mmol, 1.2 eq.), tert-butyl 4-(4-bromophenyl)-4-cyano-butanoate 1′ (500.0 mg, 1.542 mmol, 1.0 eq.), XPhos (110.28 mg, 0.231 mmol, 0.15 eq.), Pd₂(dba)₃ (141.22 mg, 0.154 mmol, 0.1 eq.) and dry 1,4-Dioxane (7.7 mL, 0.2 M). The mixture was degassed by sparging with nitrogen for 10 minutes. The tube was sealed and the mixture was stirred at 90° C. for 16 hours. The reaction was filtered over celite, washed with EtOAc and the filtrate was evaporated. The residue was purified by normal phase flash chromatography (80 g silica column, elution: 0 to 50% Heptane/EtOAc over 15 CV, product exited at 45% EtOAc, was the only one who absorbs at 254 nm). Fractions were combined and concentrated to give tert-butyl 4-[4-[4-[tert-butoxycarbonyl(methyl)amino]-1-piperidyl]phenyl]-4-cyano-butanoate 3′ (660 mg, 83% yield) as an orange oil.

LCMS method 1: retention time: 2.061 min, 88.3% purity at 215 nm, [M+H]⁺=458.2.

¹H NMR (400 MHz, DMSO-d6) δ ppm 0.79-0.88 (m, 1H), 1.21-1.28 (m, 1H), 1.36-1.41 (m, 18H), 1.60 (br d, J=9.5 Hz, 2H), 1.68-1.79 (m, 2H), 1.92-2.09 (m, 2H), 2.21-2.29 (m, 2H), 2.67 (s, 3H), 2.70-2.75 (m, 1H), 3.79 (br d, J=12.7 Hz, 2H), 4.07 (t, J=7.5 Hz, 1H), 6.97 (d, J=8.6 Hz, 2H), 7.18 (d, J=8.8 Hz, 2H).

Step 2′. Preparation of 3-[4-[4-(Methylamino)-1-piperidyl]phenyl]piperidine-2,6-dione sulfuric acid (C-29): To an aqueous mixture of tert-butyl 4-[4-[4-[tert-butoxycarbonyl(methyl)amino]-1-piperidyl]phenyl]-4-cyano-butanoate 3′ (866. mg, 1.892 mmol) in acetic acid (9.5 mL, 0.2 M) was added concentrated H₂SO₄ (0.3 mL, 5.677 mmol, 3.0 eq.). The reaction mixture was stirred vigorously at 120° C. for 1 hour, then concentrated. The crude material was dissolved in a minimal amount of water and purified by reverse-phase chromatography column using a 40 g C18 column (elution: 0% MeCN/0.1% HCOOH over 4 CV, then 0% to 10% MeCN/0.1% HCOOH over 15 CV, product exited at 5 MeCN). The pure fractions were combined and concentrated to give 3-[4-[4-(methylamino)-1-piperidyl]phenyl]piperidine-2,6-dione sulfuric acid C-29 (265 mg, 34% yield) as a yellow oil.

LCMS method 1: retention time: 0.588 min, 97.7% purity at 215 nm, [M+H]⁺=302.2.

¹H NMR (400 MHz, DMSO-d6) δ ppm 1.49-1.62 (m, 2H), 1.96-2.05 (m, 2H), 2.10-2.18 (m, 1H), 2.42-2.48 (m, 1H), 2.56 (s, 3H), 2.59-2.75 (m, 3H), 3.10 (br t, J=11.4 Hz, 1H), 3.67-3.83 (m, 3H), 4.76 (br d, J=15.9 Hz, 1H), 6.91 (br d, J=8.6 Hz, 2H), 7.05 (br d, J=8.3 Hz, 2H), 8.24 (s, 1H), 10.77 (br s, 1H).

### Example S9. Acetic acid; 3-(5-piperazin-1-yl-2-pyridyl)piperidine-2,6-dione (C-37)

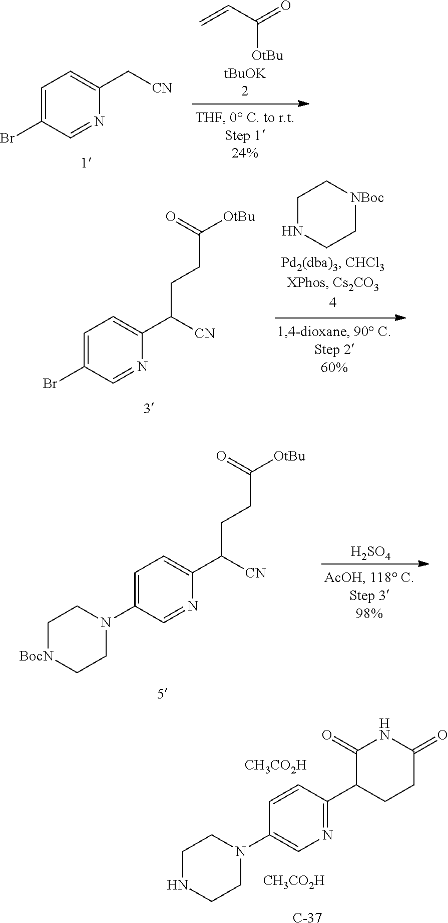

Step 1′. Preparation of tert-butyl 4-(5-bromo-2-pyridyl)-4-cyano-butanoate (3′): A solution of 2-(5-bromo-2-pyridyl)acetonitrile 1′ (1.20 g, 6.09 mmol, 1 eq.) and tert-butyl prop-2-enoate 2′ (820 mg, 6.39 mmol, 1.05 eq.) in anhydrous THF (12 mL) was cooled down to ° C. under nitrogen. tBuOK (74 mg, 0.61 mmol, 0.1 eq.) was added and the reaction was stirred at 0° C. (keeping the ice bath slowly warming up to room temperature). After 16 h, LCMS showed conversion not complete. The reaction mixture was filtered over a pad of Celite, rinsed with EtOAc and the filtrate was concentrated to dryness. The residue was purified by reverse phase flash chromatography (100 g C18 column, liquid deposit (DMSO), elution: 5% MeCN/0.1% HCOOH over 3 CV, then 5 to 40% MeCN/0.1% HCOOH over 2 CV, then 40 to 100% MeCN/0.1% HCOOH over 14 CV). Fractions were combined and partially concentrated to dryness. The suspension was then partitioned between saturated aqueous NaHCO₃ and EtOAc. The layers were separated and the aqueous one was back extracted with EtOAc. The combined organics were dried over Na₂SO₄, filtered and concentrated to dryness to give 3′ (466 mg, 24% yield) as a brown oil.

LCMS method 1: 96.6% purity at 215 nm, [M−tBu+H]⁺=269.0.

¹H NMR (400 MHz, CDCl₃) δ ppm 1.46 (s, 9H), 2.20-2.37 (m, 2H), 2.38-2.54 (m, 2H), 4.13 (dd, J=8.4, 6.2 Hz, 1H), 7.34 (d, J=8.3 Hz, 1H), 7.87 (dd, J=8.3, 2.4 Hz, 1H), 8.67 (d, J=2.0 Hz, 1H).

Step 2′. Preparation of tert-butyl 4-[6-(4-tert-butoxy-1-cyano-4-oxo-butyl)-3-pyridyl]piperazine-1-carboxylate (5′): To a sealed tube were added tert-butyl 4-(5-bromo-2-pyridyl)-4-cyano-butanoate 3′ (336 mg, 1.03 mmol, 1 eq.), tert-butyl piperazine-1-carboxylate 4′ (229 mg, 1.23 mmol, 1.2 eq.), Cs₂CO₃ (664 mg, 2.04 mmol, 2 eq.), XPhos (102 mg, 0.21 mmol, 0.2 eq.) and 1,4-dioxane (10 mL). The reaction mixture was sparged with nitrogen for 10 min and Pd₂(dba)₃·CHCl₃ (98 mg, 0.09 mmol, 0.1 eq.) was quickly added. The reaction mixture was sparged with nitrogen for a further 10 min and it was then stirred at 90° C. After 14.5 h, LCMS showed full conversion. The reaction mixture was filtered over a pad of Celite, rinsed with EtOAc and the filtrate was concentrated to dryness. The residue was purified by reverse phase flash chromatography (100 g C18 column, liquid deposit (DMSO), elution: 5% MeCN/0.1% HCOOH over 3 CV, then 5 to 30% MeCN/0.1% HCOOH over 2 CV, then 30 to 80% MeCN/0.1% HCOOH over 17 CV). Fractions were combined and partially concentrated to dryness. The suspension was then partitioned between saturated aqueous NaHCO₃ and EtOAc. The layers were separated and the aqueous one was back extracted with EtOAc. The combined organics were dried over Na₂SO₄, filtered and concentrated to dryness to give 5′ (285 mg, 60% yield) as an orange oil.

LCMS method 1: 99.9% purity at 215 nm, [M+H]⁺=431.3.

¹H NMR (400 MHz, CDCl₃) δ ppm 1.45 (s, 9H), 1.49 (s, 9H), 2.22-2.34 (m, 2H), 2.36-2.49 (m, 2H), 3.16-3.22 (m, 4H), 3.58-3.63 (m, 4H), 4.05 (t, J=7.3 Hz, 1H), 7.16-7.22 (m, 1H), 7.24-7.30 (m, 1H), 8.27 (d, J=2.7 Hz, 1H).

Step 3′. Preparation of acetic acid;3-(5-piperazin-1-yl-2-pyridyl)piperidine-2,6-dione (C-37): To a sealed tube were added tert-butyl 4-[6-(4-tert-butoxy-1-cyano-4-oxo-butyl)-3-pyridyl]piperazine-1-carboxylate 5′ (280 mg, 0.65 mmol, 1 eq.), acetic acid (3.3 mL) and concentrated sulfuric acid (255 mg, 2.60 mmol, 4 eq.). The reaction mixture was stirred at 118° C. After 70 min, LCMS showed full conversion. Volatiles were removed under reduce pressure and the residue was purified by reverse phase flash chromatography (20 g C18 Aq column, liquid deposit (H₂O), elution: 0% MeCN/0.1% HCOOH over 8 CV). Fractions were combined and concentrated to give C-37 (300 mg, 98% yield) as a yellow solid, which was used as-is for the next step.

LCMS method 1: 85.5% purity at 215 nm, [M−2CH3COOH+H]⁺=275.1.

CBM intermediates prepared using General Procedure CBM-4 are summarized in Table 5.

TABLE 5
CBM Intermediates Prepared via General Procedure CBM-4

| | Structure | Amine |
|---|---|---|
| C-12 | 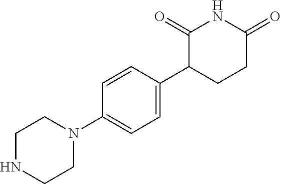 [SMILES: O=C1CCC(c2ccc(N3CCNCC3)cc2)C(=O)N1] | 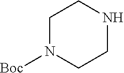 [SMILES: CC(C)(C)OC(=O)N1CCNCC1] |
| C-14 | 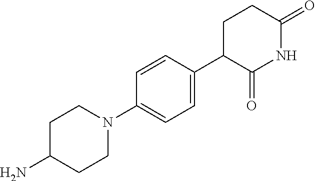 [SMILES: NC1CCN(c2ccc(C3CCC(=O)NC3=O)cc2)CC1] | 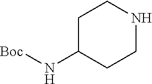 [SMILES: CC(C)(C)OC(=O)NC1CCNCC1] |
| C-19 | 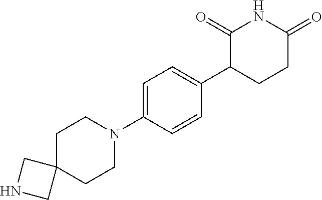 [SMILES: O=C1CCC(c2ccc(N3CCC4(CC3)CNC4)cc2)C(=O)N1] | 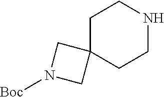 [SMILES: CC(C)(C)OC(=O)N1CC2(CCNCC2)C1] |
| C-26 | 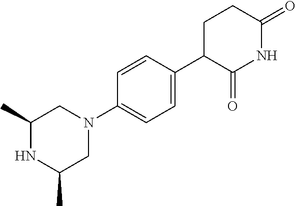 [SMILES: C[C@@H]1CN(c2ccc(C3CCC(=O)NC3=O)cc2)C[C@H](C)N1] | 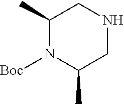 |
| C-28 | 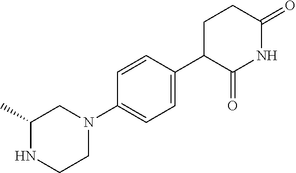 [SMILES: C[C@@H]1CN(c2ccc(C3CCC(=O)NC3=O)cc2)CCN1] | 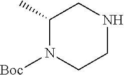 |
| C-29 | 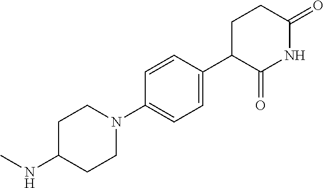 [SMILES: CNC1CCN(c2ccc(C3CCC(=O)NC3=O)cc2)CC1] | 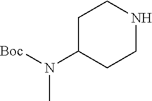 |
| C-30 | 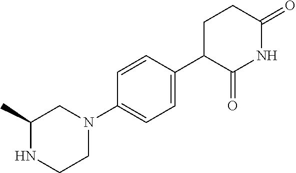 [SMILES: C[C@H]1CN(c2ccc(C3CCC(=O)NC3=O)cc2)CCN1] | 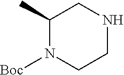 |
| C31 | 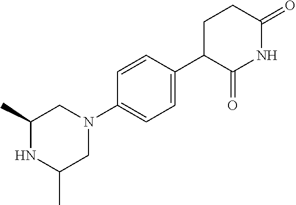 [SMILES: CC1CN(c2ccc(C3CCC(=O)NC3=O)cc2)C[C@H](C)N1] | 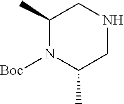 |
| C32 | 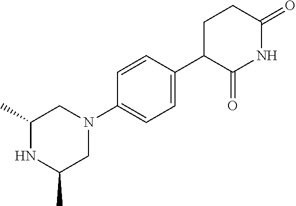 [SMILES: C[C@@H]1CN(c2ccc(C3CCC(=O)NC3=O)cc2)C[C@@H](C)N1] | 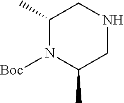 |
| C34 | 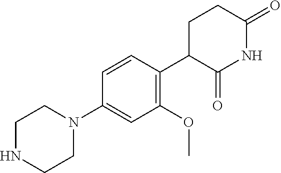 [SMILES: COc1cc(N2CCNCC2)ccc1C1CCC(=O)NC1=O] | 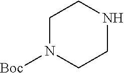 [SMILES: CC(C)(C)OC(=O)N1CCNCC1] |
| C35 | 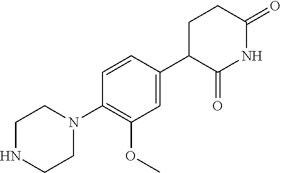 [SMILES: COc1cc(C2CCC(=O)NC2=O)ccc1N1CCNCC1] | 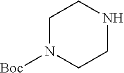 [SMILES: CC(C)(C)OC(=O)N1CCNCC1] |
| C36 | 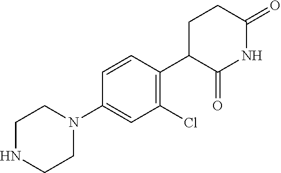 [SMILES: O=C1CCC(c2ccc(N3CCNCC3)cc2Cl)C(=O)N1] | 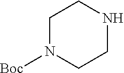 [SMILES: CC(C)(C)OC(=O)N1CCNCC1] |
| C37 | 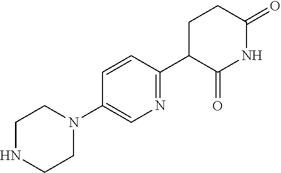 [SMILES: O=C1CCC(c2ccc(N3CCNCC3)cn2)C(=O)N1] | 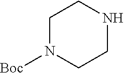 [SMILES: CC(C)(C)OC(=O)N1CCNCC1] |
| C39 | 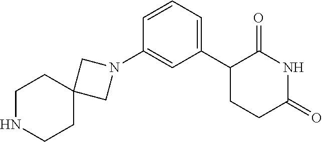 [SMILES: O=C1CCC(c2cccc(N3CC4(CCNCC4)C3)c2)C(=O)N1] | 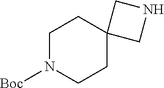 [SMILES: CC(C)(C)OC(=O)N1CCC2(CC1)CNC2] |
| | Use of 3-bromophenyl | |
| | glutarimide | |
| C-41 | 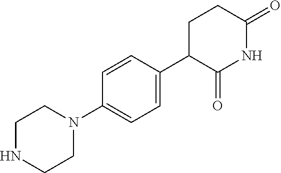 [SMILES: O=C1CCC(c2ccc(N3CCNCC3)cc2)C(=O)N1] | |
| | Chiral separation of C-12 | |
| C42 | 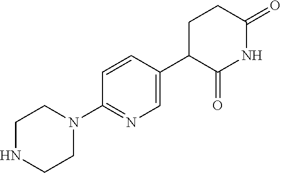 [SMILES: O=C1CCC(c2ccc(N3CCNCC3)nc2)C(=O)N1] | 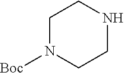 [SMILES: CC(C)(C)OC(=O)N1CCNCC1] |
| C43 | 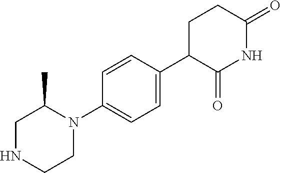 [SMILES: C[C@@H]1CNCCN1c1ccc(C2CCC(=O)NC2=O)cc1] | 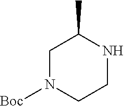 |
| C44 | 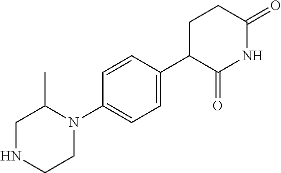 [SMILES: CC1CNCCN1c1ccc(C2CCC(=O)NC2=O)cc1] | 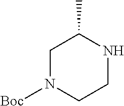 |
| C45 | 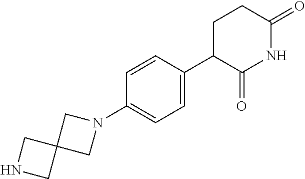 [SMILES: O=C1CCC(c2ccc(N3CC4(CNC4)C3)cc2)C(=O)N1] | 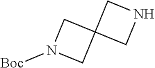 [SMILES: CC(C)(C)OC(=O)N1CC2(CNC2)C1] |
| C48 | 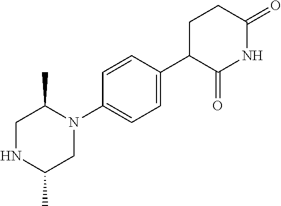 [SMILES: C[C@@H]1CN[C@@H](C)CN1c1ccc(C2CCC(=O)NC2=O)cc1] | 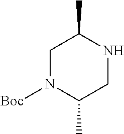 |
| C49 | 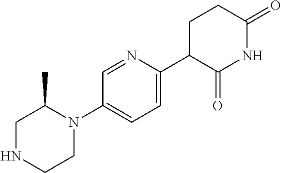 [SMILES: C[C@@H]1CNCCN1c1ccc(C2CCC(=O)NC2=O)nc1] | 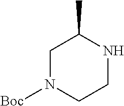 |
| C50 | 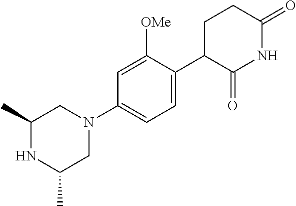 [SMILES: COc1cc(N2C[C@H](C)N[C@@H](C)C2)ccc1C1CCC(=O)NC1=O] | 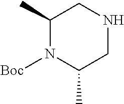 |
| C51 | 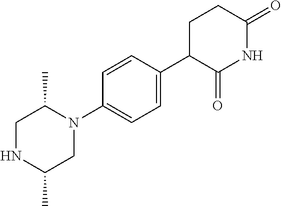 [SMILES: C[C@H]1CN(c2ccc(C3CCC(=O)NC3=O)cc2)[C@@H](C)CN1] | 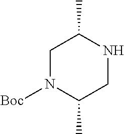 |
| C52 | 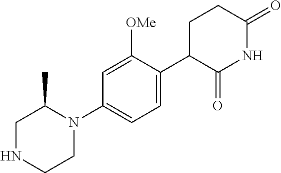 [SMILES: COc1cc(N2CCNC[C@H]2C)ccc1C1CCC(=O)NC1=O] | 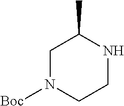 |
| C53 | 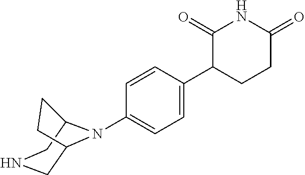 [SMILES: O=C1CCC(c2ccc(N3C4CCC3CNC4)cc2)C(=O)N1] | 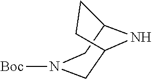 [SMILES: CC(C)(C)OC(=O)N1CC2CCC(C1)N2] |
| C54 | 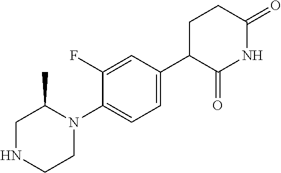 [SMILES: C[C@@H]1CNCCN1c1ccc(C2CCC(=O)NC2=O)cc1F] | 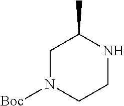 |
| C55 | 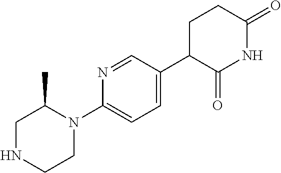 [SMILES: C[C@@H]1CNCCN1c1ccc(C2CCC(=O)NC2=O)cn1] | 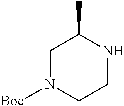 |
| C56 | 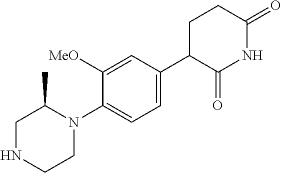 [SMILES: COc1cc(C2CCC(=O)NC2=O)ccc1N1CCNC[C@H]1C] | 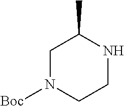 |
| C57 | 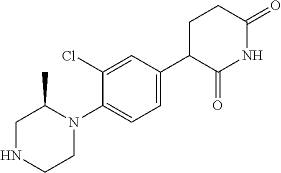 [SMILES: C[C@@H]1CNCCN1c1ccc(C2CCC(=O)NC2=O)cc1Cl] | 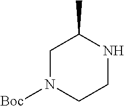 |
| C58 | 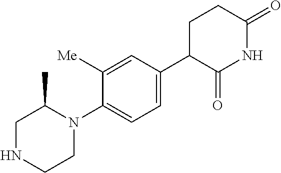 [SMILES: Cc1cc(C2CCC(=O)NC2=O)ccc1N1CCNC[C@H]1C] | 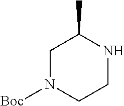 |
| C59 | 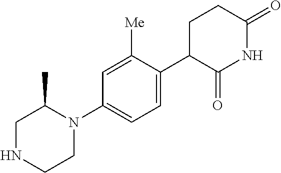 [SMILES: Cc1cc(N2CCNC[C@H]2C)ccc1C1CCC(=O)NC1=O] | 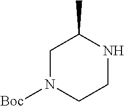 |
| C60 | 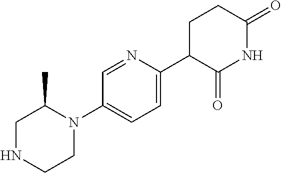 [SMILES: C[C@@H]1CNCCN1c1ccc(C2CCC(=O)NC2=O)nc1] | 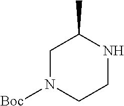 |
| C61 | 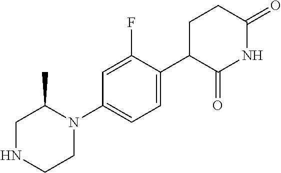 [SMILES: C[C@@H]1CNCCN1c1ccc(C2CCC(=O)NC2=O)c(F)c1] | 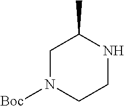 |
| C62 | 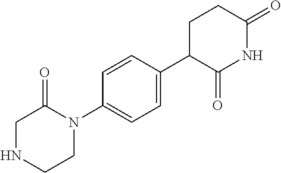 [SMILES: O=C1CCC(c2ccc(N3CCNCC3=O)cc2)C(=O)N1] | 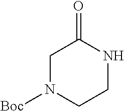 [SMILES: CC(C)(C)OC(=O)N1CCNC(=O)C1] |
| C63 | 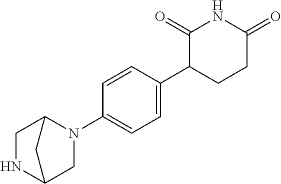 [SMILES: O=C1CCC(c2ccc(N3CC4CC3CN4)cc2)C(=O)N1] | 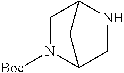 [SMILES: CC(C)(C)OC(=O)N1CC2CC1CN2] |
| C64 | 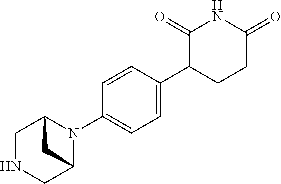 [SMILES: O=C1CCC(c2ccc(N3[C@@H]4CNC[C@H]3C4)cc2)C(=O)N1] | 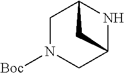 [SMILES: CC(C)(C)OC(=O)N1C[C@H]2C[C@@H](C1)N2] |
| C65 | 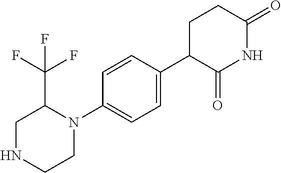 [SMILES: O=C1CCC(c2ccc(N3CCNCC3C(F)(F)F)cc2)C(=O)N1] | 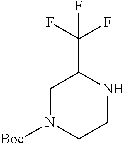 [SMILES: CC(C)(C)OC(=O)N1CCNC(C(F)(F)F)C1] |
| C66 | 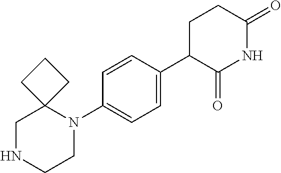 [SMILES: O=C1CCC(c2ccc(N3CCNCC34CCC4)cc2)C(=O)N1] | 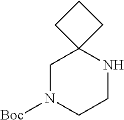 [SMILES: CC(C)(C)OC(=O)N1CCNC2(CCC2)C1] |
| C67 | 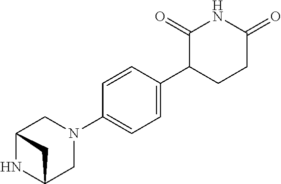 [SMILES: O=C1CCC(c2ccc(N3C[C@H]4C[C@@H](C3)N4)cc2)C(=O)N1] | 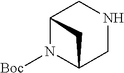 [SMILES: CC(C)(C)OC(=O)N1[C@@H]2CNC[C@H]1C2] |
| C68 | 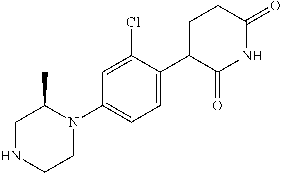 [SMILES: C[C@@H]1CNCCN1c1ccc(C2CCC(=O)NC2=O)c(Cl)c1] | 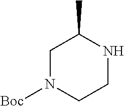 |
| C69 | 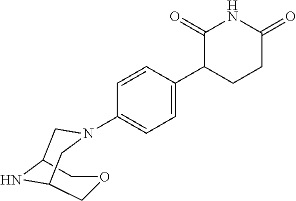 [SMILES: O=C1CCC(c2ccc(N3CC4COCC(C3)N4)cc2)C(=O)N1] | 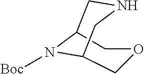 [SMILES: CC(C)(C)OC(=O)N1C2CNCC1COC2] |
| C70 | 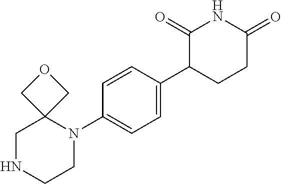 [SMILES: O=C1CCC(c2ccc(N3CCNCC34COC4)cc2)C(=O)N1] | 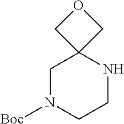 [SMILES: CC(C)(C)OC(=O)N1CCNC2(COC2)C1] |
| C71 | 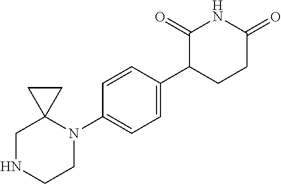 [SMILES: O=C1CCC(c2ccc(N3CCNCC34CC4)cc2)C(=O)N1] | 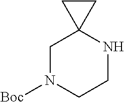 [SMILES: CC(C)(C)OC(=O)N1CCNC2(CC2)C1] |
| C72 | 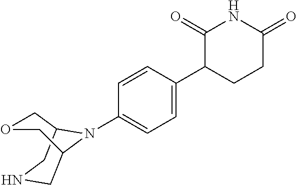 [SMILES: O=C1CCC(c2ccc(N3C4CNCC3COC4)cc2)C(=O)N1] | 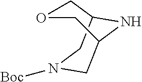 [SMILES: CC(C)(C)OC(=O)N1CC2COCC(C1)N2] |
| C74 | 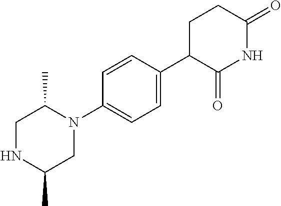 [SMILES: C[C@@H]1CN(c2ccc(C3CCC(=O)NC3=O)cc2)[C@@H](C)CN1] | 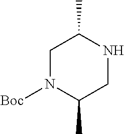 |
| C-77 | 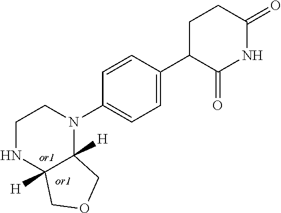 [SMILES: O=C1CCC(c2ccc(N3CCN[C@H]4COC[C@H]43)cc2)C(=O)N1] | 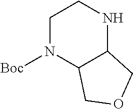 [SMILES: CC(C)(C)OC(=O)N1CCNC2COCC21] |
| | Chiral separation | |
| C-77i | 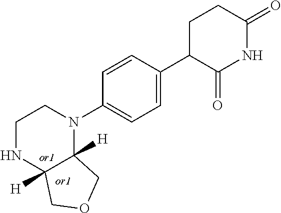 [SMILES: O=C1CCC(c2ccc(N3CCN[C@H]4COC[C@H]43)cc2)C(=O)N1] | 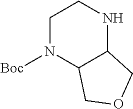 [SMILES: CC(C)(C)OC(=O)N1CCNC2COCC21] |
| | Chiral separation | |

### General Procedure CBM-5

### Example S10. 3-[4-[2-(4-Piperidyl)ethynyl]phenyl]piperidine-2,6-dione hydrochloride (C-21)

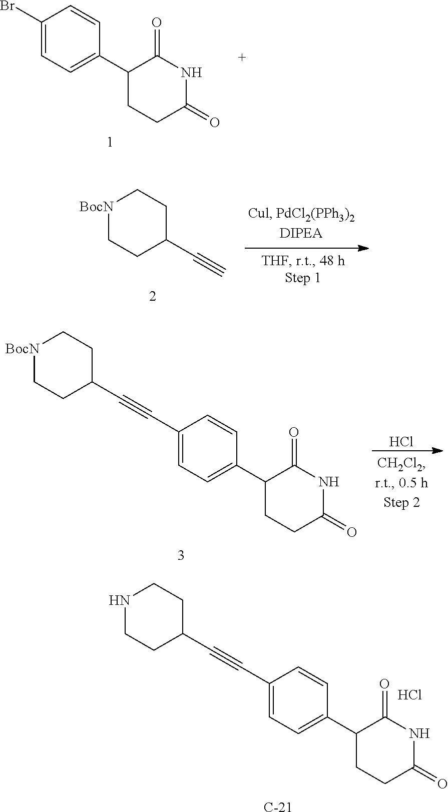

Step 1. Preparation of Tert-butyl 4-[2-[4-(2,6-dioxo-3-piperidyl)phenyl]ethynyl]piperidine-1-carboxylate (3): A solution of 3-(4-bromophenyl)piperidine-2,6-dione 1 (250 mg, 0.932 mmol, 1.0 eq.), tert-butyl 4-ethynylpiperidine-1-carboxylate 2 (234.1 mg, 1.118 mmol, 1.2 eq.), CuI (17.76 mg, 0.093 mmol, 0.1 eq.), DIPEA (1.62 mL, 9.320 mmol, 10.0 eq.) and PdCl₂(PPh₃)₂ (65.45 mg, 0.093 mmol, 0.1 eq.) in THF (6.2 mL, 0.15 M) was degassed by sparging with nitrogen for 20 minutes. The reaction was stirred at room temperature for 48 hours. The reaction was filtered over celite, washed with MeTHF and the filtrate was evaporated. The residue was purified by normal phase flash chromatography (40 g silica column, elution: 0 to 20% CH₂Cl₂/EtOAc over 15 CV, product exited at 7% EtOAc). Fractions were combined and concentrated to give tert-butyl 4-[2-[4-(2,6-dioxo-3-piperidyl)phenyl]ethynyl]piperidine-1-carboxylate 3 (183 mg, 49% yield) as yellow solid.

LCMS method 1: retention time: 1.820 min, 92.8% purity at 215 nm, [M−Boc+H]⁺=297.2; [M−tBu+H]⁺=341.2; [M+Na]⁺=419.2.

¹H NMR (400 MHz, DMSO-d6) δ ppm 1.40 (s, 9H), 1.44-1.55 (m, 3H), 1.76-1.85 (m, 2H), 1.96-2.05 (m, 1H), 2.13-2.26 (m, 1H), 2.60-2.71 (m, 1H), 2.81-2.90 (m, 1H), 3.14 (br t, J=9.7 Hz, 2H), 3.59-3.68 (m, 2H), 3.88 (dd, J=11.7, 4.9 Hz, 1H), 7.20 (d, J=8.1 Hz, 2H), 7.36 (d, J=8.3 Hz, 2H), 10.84 (s, 1H).

Step 2. Preparation of 3-[4-[2-(4-Piperidyl)ethynyl]phenyl]piperidine-2,6-dione hydrochloride (C-21): Under nitrogen, a mixture of tert-butyl 4-[2-[4-(2,6-dioxo-3-piperidyl)phenyl]ethynyl]piperidine-1-carboxylate 3 (180. mg, 0.454 mmol, 1.0 eq.) and 4 M HCl in 1,4-dioxane (1.5 mL, 6.0 mmol, 13.0 eq.) in DCM (4.5 mL, 0.1 M) was stirred at room temperature for 0.5 hour. The mixture was concentrated under reduced pressure and co-evaporated 3 times with acetonitrile to give 3-[4-[2-(4-piperidyl)ethynyl]phenyl]piperidine-2,6-dione; hydrochloride C-21 (150 mg, 98% yield) as a yellow solid.

LCMS method 1: retention time: 1.121 min, 99.9% purity at 215 nm, [M−HCl+H]⁺=297.2.

¹H NMR (400 MHz, DMSO-d6) δ ppm 1.75-1.85 (m, 3H), 2.00-2.05 (m, 2H), 2.19 (qd, J=12.3, 4.4 Hz, 1H), 2.61-2.72 (m, 1H), 2.96-3.04 (m, 4H), 3.17-3.24 (m, 2H), 3.89 (dd, J=11.7, 4.9 Hz, 1H), 7.22 (d, J=8.1 Hz, 2H), 7.38 (d, J=8.3 Hz, 2H), 8.78-8.95 (m, 2H), 10.85 (s, 1H).

CBM intermediates prepared using General Procedure CBM-5 are summarized in Table 6.

TABLE 6
CBM Intermediates Prepared via General Procedure CBM-5

| | Structure |
|---|---|
| C-21 | 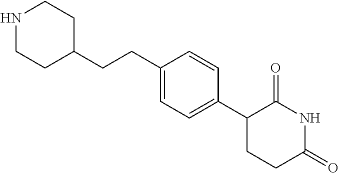 [SMILES: O=C1CCC(c2ccc(CCC3CCNCC3)cc2)C(=O)N1] |

### General Procedure CBM-6

### Example S11. 3-(4-(piperidin-4-yl)phenyl)piperidine-2,6-dione (C-25)

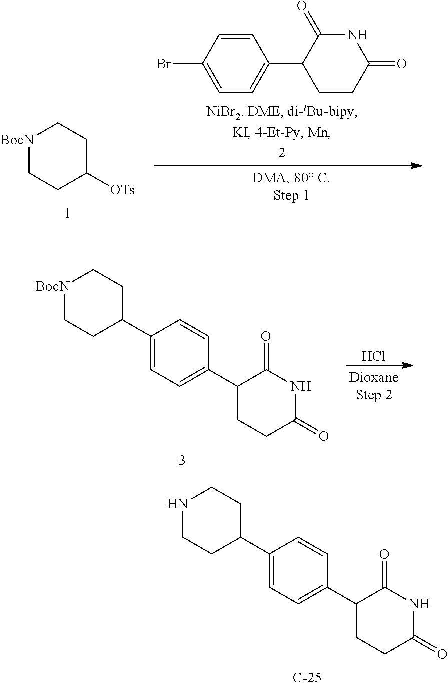

Step 1. Preparation of tert-butyl 4-[4-(2,6-dioxo-3-piperidyl)phenyl]piperidine-1-carboxylate (3): A solution of tert-butyl 4-(p-tolylsulfonyloxy)piperidine-1-carboxylate 1 (1.01 g, 2.84 mmol), 3-(4-bromophenyl)piperidine-2,6-dione 2 (772.8 mg, 2.88 mmol), 4,4′-di-tert-butyl-2,2′-dipyridyl (74.9 mg, 0.28 mmol), KI (535.7 mg, 3.23 mmol), 4-ethylpyridine (0.3 mL, 2.64 mmol) and Mn (339.1 mg, 6.18 mmol) in DMA (14 mL) was purged with nitrogen for 10 min. NiBr₂·DME (104.6 mg, 0.34 mmol) was then added and nitrogen was bubbled through the solution for 5 more min. After 19 h at 80° C., the reaction mixture was cooled down to RT and partitioned between EtOAc (50 mL) and brine (50 mL). The organic phase was separated, washed with brine (2×50 mL), dried over Na₂SO₄, filtered and concentrated in vacuo. Purification by reverse phase chromatography (C18-100 g, 5-100% MeCN/0.1% formic acid in water, 20 CV) afforded tert-butyl 4-[4-(2,6-dioxo-3-piperidyl)phenyl]piperidine-1-carboxylate 3 (504.6 mg, 1.35 mmol, 48% yield) as a slightly pink solid.

¹H NMR (400 MHz, DMSO-d6) δ ppm 1.41 (s, 9H), 1.44-1.54 (m, 2H), 1.74 (br d, J=12.6 Hz, 2H), 1.98-2.06 (m, 1H), 2.10-2.23 (m, 1H), 2.45 (br t, J=3.9 Hz, 1H), 2.60-2.71 (m, 2H), 2.73-2.91 (m, 2H), 3.81 (dd, J=11.4, 4.8 Hz, 1H), 4.07 (br d, J=11.2 Hz, 2H), 7.11-7.16 (m, 2H), 7.18-7.23 (m, 2H), 10.81 (s, 1H). LCMS (ESI+, m/z): [M−tBu+H]⁺=317.2.

Step 2. Preparation of 3-(4-(piperidin-4-yl)phenyl)piperidine-2,6-dione (C-25): To a solution of tert-butyl 4-[4-(2,6-dioxo-3-piperidyl)phenyl]piperidine-1-carboxylate 3 (137. mg, 0.3700 mmol) in 1,4-Dioxane (1.8 mL) at room temperature was added HCl in dioxane (1.8 mL, 7.2 mmol) (3:03 PM). The resulting solution was stirred at room temperature. After an hour, the reaction mixture was concentrated on the rotovap. The residue was dissolved in 1,4-Dioxane (1.8 mL), and HCl in dioxane (1.8 mL, 7.2 mmol) was added. The mixture was stirred at room temperature for 3.5 h. After HPLC analysis showed complete conversion the mixture was concentrated on the rotovap to give 119.6 mg of the product C-25 as a light-yellow solid (quant. yield). The product was 99.9% pure by HPLC (2.5 min. run, 215 nm).

Luna C18 50×3 mm 45° C. C-25 ret. time (product)=1.170 min.

CBM intermediates prepared using General Procedure CBM-6 are summarized in Table 7.

TABLE 7
CBM Intermediates Prepared via General Procedure CBM-6

| | Structure |
|---|---|
| C-25 | 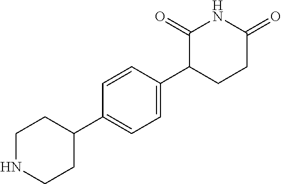 [SMILES: O=C1CCC(c2ccc(C3CCNCC3)cc2)C(=O)N1] |

### General Procedure CBM-7

### Example S12. 3-[4-(4-piperidylmethoxy)phenyl]piperidine-2,6-dione;2,2,2-trifluoroacetic acid (C-33)

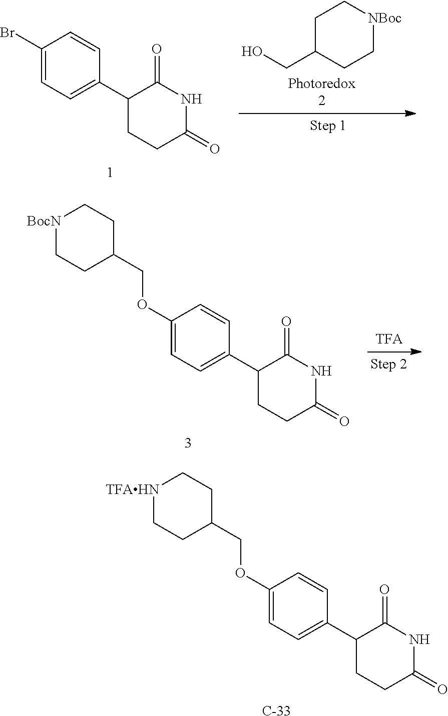

Step 1. Preparation of tert-butyl 4-[[4-(2,6-dioxo-3-piperidyl)phenoxy]methyl]piperidine-1-carboxylate (3): To a Schlenk flask were added Quinuclidine (8.29 mg, 0.07 mmol, 0.1 eq.), NiCl₂·DME (8.2 mg, 0.04 mmol, 0.05 eq.), 3-(4-bromophenyl)piperidine-2,6-dione 1 (200 mg, 0.75 mmol, 1 eq.), Ir[dF(CF₃)ppy]₂(dtbbpy)PF₆ (8.37 mg, 0.01 mmol, 0.01 eq.), K₂CO₃ (103.1 mg, 0.75 mmol, 1 eq.) and dtbbpy (10.01 mg, 0.04 mmol, 0.05 eq.) in MeCN (5 mL, 0.15 M). The flask was placed under nitrogen and then tert-butyl 4-(hydroxymethyl)piperidine-1-carboxylate 2 (401.5 mg, 1.86 mmol, 2 eq.) was added. The Schlenk was then frozen at −78° C., put on the high vacuum pump under nitrogen and heated slowly to room temperature with a water bath. This procedure was made three times, making sure that there was no oxygen left in the Schlenk tube. The tube was then irradiated under blue LEDs for the week-end. After three days, LCMS showed major conversion into compound 3. The solvent was evaporated under reduce pressure and the crude mixture was purified by reverse phase FC purification (50 g C18 gold column, liquid deposit (DMSO), 5% MeOH/0.1% HCOOH over 4 CV, then 5 to 80% MeOH/0.1% HCOOH over 10 CV, then 80% MeOH/0.1% HCOOH over 4 CV, the product came out at 80% MeOH). Fractions were combined and concentrated to give 3 (126 mg, 42% yield) as a yellow solid.

LCMS method 1: 99.9% purity at 215 nm, [M-t-Bu+H]⁺=347.2.

¹H NMR (400 MHz, chloroform-d) δ ppm 1.13-1.35 (m, 4H), 1.46-1.48 (m, 9H), 1.82 (br d, J=14.7 Hz, 2H), 1.90-2.03 (m, 1H), 2.18-2.32 (m, 2H), 2.60-2.81 (m, 4H), 3.74 (dd, J=9.5, 5.4 Hz, 1H), 3.80 (d, J=6.4 Hz, 2H), 6.89 (d, J=8.6 Hz, 2H), 7.13 (d, J=8.6 Hz, 2H), 7.92 (br s, 1H).

Step 2. Preparation of 3-[4-(4-piperidylmethoxy)phenyl]piperidine-2,6-dione;2,2,2-trifluoroacetic acid (C-33): To a solution of tert-butyl 4-[[4-(2,6-dioxo-3-piperidyl)phenoxy]methyl]piperidine-1-carboxylate 3 (126 mg, 0.31 mmol, 1 eq.) in DCM (1 mL, 0.31 M) was added TFA (0.36 mL, 4.7 mmol, 15 eq.). The reaction mixture was stirred at room temperature. After 2 h, LCMS showed full conversion into compound 4. The reaction mixture was concentrated under reduced pressure and the residue was co-evaporated with Toluene (2×) and MeCN (2×) to give C-33 (157 mg, quantitative yield) as a yellow solid as a trifluoroacetic acid salt.

LCMS method 1: 91.8% purity at 215 nm, [M-CF₃COOH+H]⁺=303.2.

¹H NMR (400 MHz, DMSO-d6) δ ppm 1.37-1.52 (m, 2H), 1.88-1.96 (m, 2H), 1.96-2.20 (m, 3H), 2.43-2.48 (m, 1H), 2.60-2.70 (m, 1H), 2.84-2.97 (m, 2H), 3.28-3.33 (m, 1H), 3.73-3.81 (m, 1H), 3.81-3.87 (m, 2H), 6.89 (d, J=8.6 Hz, 2H), 7.12 (d, J=8.6 Hz, 2H), 8.14-8.29 (m, 1H), 8.48-8.61 (m, 1H), 10.78 (s, 1H). One proton was missing in the ¹H NMR spectrum.

CBM intermediates prepared using General Procedure CBM-7 are summarized in Table 8.

TABLE 8
CBM Intermediates Prepared via General Procedure CBM-7

| | Structure |
|---|---|
| C-33 | 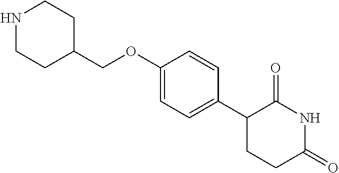 [SMILES: O=C1CCC(c2ccc(OCC3CCNCC3)cc2)C(=O)N1] |

### General Procedure CBM-8

### Example S13. 3-[4-[4-(hydroxymethyl)-1-piperidyl]phenyl]piperidine-2,6-dione (C-38)

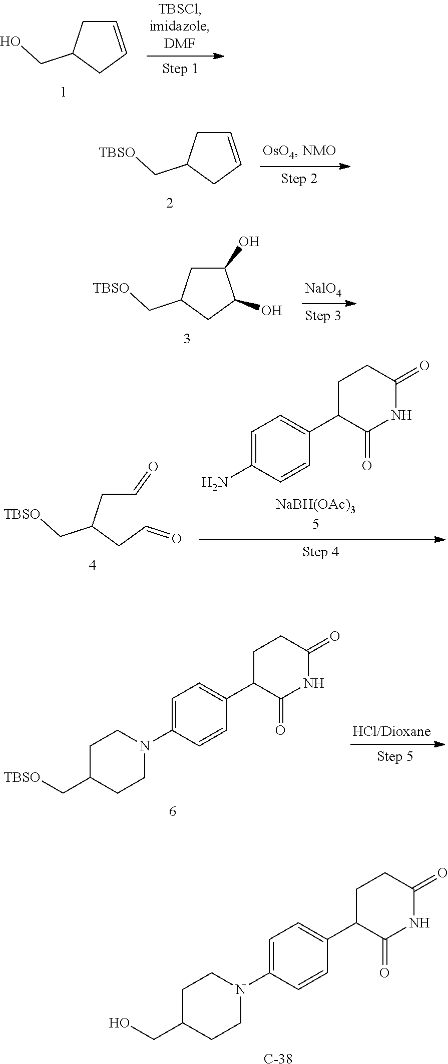

Step 1. Preparation of tert-butyl-(cyclopent-3-en-1-ylmethoxy)-dimethyl-silane (2): To a solution of Cyclopent-3-en-1-ylmethanol 1 (1 g, 10.19 mmol, 1eq.) in DMF (4 mL, 2.5 M) was added imidazole (0.83 g, 12.23 mmol, 1.2 eq.) and tert-Butyldimethylsilyl chloride (2.0 g, 13.25 mmol, 1.3 eq.). After stirring for 30 minutes at room temperature under nitrogen atmosphere, TLC showed full conversion into compound 2. Water was added to the reaction mixture and it was extracted twice with EtOAc. The organic phases were washed twice with water and then with brine, dried over sodium sulfate, filtered and evaporated under reduced pressure. The crude mixture was then purified by normal phase flash chromatography (80 g gold column, solid deposit, elution 0% to 40% Hept./EtOAc over 20 CV, product came out at 10% EtOAc). Fractions were combined and concentrated, affording 2 (2.28 g, quantitative yield) as a colorless oil.

¹H NMR (400 MHz, chloroform-d) δ ppm 0.05 (s, 6H), 0.90 (s, 9H), 2.04-2.19 (m, 2H), 2.35-2.52 (m, 3H), 3.49 (d, J=6.8 Hz, 2H), 5.65 (s, 2H).

Step 2. Preparation of (5R)-3-[[tert-butyl(dimethyl)silyl]oxymethyl]hexane-1,5-diol (3): Under nitrogen, a solution of tert-butyl-(cyclopent-3-en-1-ylmethoxy)-dimethyl-silane 2 (2.2 g, 10.36 mmol, 1 eq.) in t-BuOH (35 mL, 0.2 M) and THF (17.5 mL, 0.2 M) was stirred at room temperature for 5 minutes, then OsO4 (0.66 mL, 4.0% w/w in H₂O, 0.104 mmol, 0.01 eq.) and NMO (1.46 g, 12.43 mmol, 1.2 eq.) were successively added. After stirring for 18 h. at room temperature under nitrogen atmosphere, TLC showed full conversion into compound 3. The mixture was concentrated to remove t-BuOH and THF, then the residue was dissolved in ethyl acetate (150 mL) and washed with 10% aq. Na2SO3 (2×15 mL), sat. aq. NaHCO₃ (15 mL) and sat. aq. NaCl (15 mL). The organic phase was dried over sodium sulfate, filtered and concentrated to dryness. The residue was then purified by normal phase flash chromatography (80 g silica column, elution: 0 to 10% methanol/dichloromethane over 14 CV, product exited at 10% methanol). Fractions were combined and concentrated to give 3 (2.44 g, 88% yield) as a brown oil.

¹H NMR (400 MHz, chloroform-d) δ ppm 0.04 (s, 6H), 0.89 (s, 9H), 1.64-1.74 (m, 2H), 1.75-1.87 (m, 2H), 2.40-2.53 (m, 1H), 3.47 (d, J=5.4 Hz, 2H), 4.09-4.17 (m, 2H).

Step 3. Preparation of 3-[[tert-butyl)dimethyl)silyl]oxymethyl]pentanedial (4): Under nitrogen, a solution of (1S,2R)-4-[[tert-butyl(dimethyl)silyl]oxymethyl]cyclopentane-1,2-diol 3 (2.4 g, 9.74 mmol, 1 eq.) in THF (35 mL, 0.19 M) and H₂O (17.5 mL, 0.19 M) was stirred at room temperature for 5 minutes, then NaIO4 (2.5 g, 11.69 mmol, 1.2 eq.) was added. After stirring for 1 h. at room temperature under nitrogen atmosphere, TLC (CH₂Cl₂/MeoH 95:5, KMnO₄ stain) showed full conversion into compound 4. The mixture was concentrated to remove THF, then brine (40 mL) was added. The aqueous phase was then extracted 3 times with ethyl acetate (3×75 mL). The combined organic phases were dried over sodium sulfate, filtered and concentrated under reduced pressure. The residue was dissolved in dichloromethane (150 mL) and stirred for 16 h. at room temperature in the presence of a large excess of magnesium sulfate. The solid was filtered, then the organic phase was concentrated and dried under high vacuum to give 4 (2.04 g, 86% yield) as a colorless oil.

¹H NMR (400 MHz, chloroform-d) δ ppm 0.03 (s, 6H), 0.88 (s, 9H), 2.41-2.49 (m, 2H), 2.51-2.60 (m, 2H), 2.69-2.82 (m, 1H), 3.57 (d, J=5.6 Hz, 2H), 9.78 (s, 2H).

Step 4. Preparation of 3-[4-[4-[[tert-butyl)dimethyl)silyl]oxymethyl]-1-piperidyl]phenyl]piperidine-2,6-dione (6): To a solution of 3-(4-aminophenyl)piperidine-2,6-dione 5 (250 mg, 1.22 mmol, 1 eq.) and 3-[[tert-butyl(dimethyl)silyl]oxymethyl]pentanedial 4 (360 mg, 1.47 mmol, 1.2 eq.) in DCE (12.2 mL, 0.1 M) was added NaBH(OAc)₃ (649 mg, 3.06 mmol, 2.5 eq.). The mixture was stirred at room temperature. After 2 h, LCMS showed complete conversion into compound 6. The reaction mixture was partitioned between DCM (20 mL) and sat. NaHCO₃ (15 mL). The organic phase was separated and the aqueous layer was extracted with DCM (2×30 mL). The combined organic phases were dried over sodium sulfate, filtrated and evaporated under reduced pressure, affording 6 (413 mg, 81% yield) as a white solid that was used as is without further purification.

LCMS method 1: 99.9% purity at 215 nm, [M+H]⁺=417.4.

¹H NMR (400 MHz, chloroform-d) δ ppm 0.06 (s, 6H), 0.91 (s, 9H), 1.28-1.41 (m, 2H), 1.61-1.68 (m, 1H), 1.82 (br d, J=12.5 Hz, 2H), 2.19-2.33 (m, 2H), 2.57-2.77 (m, 4H), 3.49 (d, J=6.4 Hz, 2H), 3.66-3.76 (m, 3H), 6.93 (d, J=8.8 Hz, 2H), 7.08 (d, J=8.6 Hz, 2H), 7.90 (br s, 1H).

Step 5. Preparation of 3-[4-[4-(hydroxymethyl)-1-piperidyl]phenyl]piperidine-2,6-dione (C-38): To 3-[4-[4-[[tert-butyl(dimethyl)silyl]oxymethyl]-1-piperidyl]phenyl]piperidine-2,6-dione 6 (410 mg, 0.98 mmol, 1 eq.) in DCM (12.2 mL, 0.08 M) was added 4 M HCl in dioxane (12.3 mL, 49.20 mmol, 50 eq.). The reaction mixture was stirred at rt. After 1.5 h, LCMS showed complete conversion into compound 7. The solvent was removed under reduced pressure and the residue was co-evaporated with toluene followed by MeCN. The residue was dried under high vacuum, affording C-38 (453 mg, quantitative yield) as an off-white solid that was used as is directly for the next step.

LCMS method 1: 99.9% purity at 215 nm, [M+H]⁺=303.4.

CBM intermediates prepared using General Procedure CBM-8 are summarized in Table 9.

TABLE 9
CBM Intermediates Prepared via General Procedure CBM-8

| | Structure |
|---|---|
| C-38 | 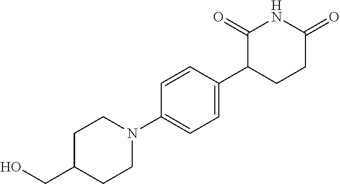 [SMILES: O=C1CCC(c2ccc(N3CCC(CO)CC3)cc2)C(=O)N1] |

### General Procedure CBM-9

### Example S14. Tert-butyl 4-(4-(3-methyl-2,6-dioxopiperidin-3-yl)phenyl)piperazine-1-carboxylate (C-40)

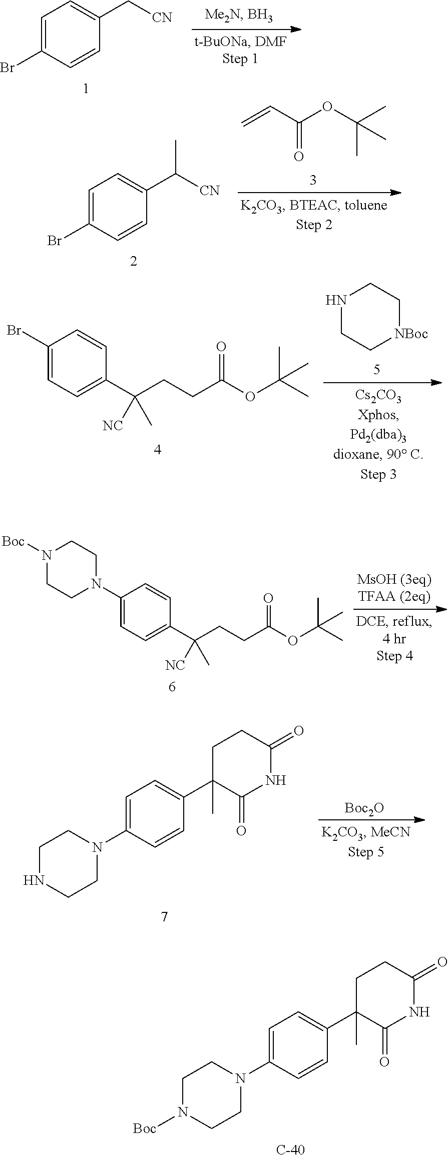

Step 1. General procedure for preparation of compound (2): To a solution of 2-(4-bromophenyl)acetonitrile 1 (100 g, 510 mmol) in DMF (1000 mL) was added Me₂NH—BH₃ (30 g, 510 mmol) and t-BuONa (73.5 g, 765 mmol). After addition, the reaction was stirred at ° C. for 1 hour. TLC (petroleum ether/ethyl acetate, 10:1) showed the staring material was consumed. The reaction mixture was quenched with water (250 mL), extracted with ethyl acetate (300 mL×2), the combined organic layer was washed with brine (200 mL), dried over anhydrous Na₂SO₄, filtered and concentrated. The residue was purified by flash chromatography give 2-(4-bromophenyl)propanenitrile 2 (49 g, 45.7% yield) as an orange oil.

¹H NMR (400 MHz, CDCl₃) δ 7.47-7.43 (m, 2H), 7.19-7.15 (m 2H), 3.82-3.77 (m 1H), 1.56 (d, J=3.2 Hz, 1H).

Step 2. General procedure for preparation of Compound (4): To a solution of 2-(4-bromophenyl)propanenitrile 2 (49 g, 233 mmol) in toluene (500 mL) was added tert-butyl prop-2-enoate 3 (59.8 g, 467 mmol), K₂CO₃ (64.4 g, 467 mmol) and benzyltriethylammonium chloride (10.6 g, 46.7 mmol). The reaction mixture was stirred at 90° C. for 16 hours. HPLC showed the starting material was consumed. The reaction mixture was cooled to RT and filtered, the cake was washed with ethyl acetate (200 mL). The combined organic layer was concentrate to give crude tert-butyl 4-(4-bromophenyl)-4-cyano-pentanoate 4 (80 g, crude) as an orange oil, which was used into next step without any further purification.

¹H NMR (400 MHz, CDCl₃) δ: 7.47-7.45 (m, 2H), 7.26-7.23 (m, 2H), 2.33-2.29 (m, 1H), 2.18-2.04 (m, 3H), 1.64 (s, 3H), 1.34 (s, 9H).

Step 3. General procedure for preparation of Compound (6): To a solution of tert-butyl 4-(4-bromophenyl)-4-cyano-pentanoate 4 (130 g, 384 mmol) and tert-butyl piperazine-1-carboxylate 5 (78.7 g, 423 mmol) in 1,4-dioxane (1500 mL) was added Cs₂CO₃ (250 g, 769 mmol), Pd₂(dba)₃ (12.3 g, 13.45 mmol) and Xphos (9.16 g, 19.2 mmol). The reaction was degassed with N₂ for 5 min and then stirred at 90° C. for 16 hrs. TLC (petroleum ether/ethyl acetate, 2:1) showed the reaction was finished. The reaction mixture was cooled to RT and filtered. The cake was washed with ethyl acetate (1000 mL), the combined organic layer was concentrated. The residue was purified by MPLC to give tert-butyl 4-[4-(4-tert-butoxy-1-cyano-1-methyl-4-oxo-butyl)phenyl]piperazine-1-carboxylate 6 (143.5 g, 84.2% yield) as a gray solid.

¹H NMR (400 MHz, CDCl₃) δ: 7.25 (t, J=8.8 Hz, 2H), 6.84 (d, J=8.8 Hz, 2H), 3.51 (t, J=4.8 Hz, 2H), 3.08 (t, J=4.8 Hz, 2H), 2.32-2.13 (m, 1H), 2.11-2.05 (m, 3H), 1.62 (s, 3H), 1.42 (s, 9H), 1.33 (s, 9H).

Step 4. General procedure for preparation of Compound (7): To a solution of tert-butyl 4-[4-(4-tert-butoxy-1-cyano-1-methyl-4-oxo-butyl)phenyl] piperazine-1-carboxylate 6 (100 g, 225 mmol) in DCE (2000 mL) was added MsOH (65 g, 676 mmol) and TFAA (127 g, 451 mmol). The reaction mixture was stirred at 90° C. for 16 hours. TLC (petroleum ether/ethyl acetate, 2:1) showed the reaction was finished. The reaction mixture was cooled to RT and concentrated. The crude 7 was used in step without any further purification and base on the theoretical amount (64.8 g) as orange oil.

Step 5. General procedure for preparation of Compound (C-40): To a solution of 3-methyl-3-(4-piperazin-1-ylphenyl)piperidine-2,6-dione 7 (64.8 g, 226 mmol) in MeCN (1000 mL) was added Boc₂O (100 g, 458 mmol) and K₂CO₃ (187 g, 1.35 mol). The reaction mixture was stirred at 20° C. for 5 hours. TLC (petroleum ether/ethyl acetate, 1:1) showed the reaction was finished. The reaction mixture was diluted with DCM (1000 mL) and EtOH (1000 mL), stirred for 1 hour and filtered, the cake was washed with DCM (1000 mL), the organic layer was concentrated to give crude product, which was combined with page 11, total 230 g crude. The mixture was diluted with ethyl acetate (2000 mL), water (1000 mL), stirred at 20° C. for 1 hour and then filtered. The cake was dried to give product 85 g (as batch 1). The mother liquor was separated and the organic layer was concentrated, the residue was purified by prep-HPLC to give product 11.2 g. Total 96.2 g of product was obtained. Base on this, 64.8 g of starting material to give product 61.8 g C-40 (yield 71%) as gray solid.

¹H NMR (400 MHz, CDCl₃) δ: 10.86 (s, 1H), 7.12 (d, J=8.8 Hz, 2H), 6.94 (d, J=8.8 Hz, 2H), 3.44 (t, J=4.8 Hz, 2H), 3.09 (t, J=4.8 Hz, 2H), 2.51-2.41 (m, 1H), 2.33-2.30 (m, 3H), 2.07-2.04 (m, 2H), 1.42 (s, 9H), 1.39 (s, 3H).

CBM intermediates prepared using General Procedure CBM-9 are summarized in Table 10.

TABLE 10
CBM Intermediates Prepared via General Procedure CBM-9

| | Structure |
|---|---|
| C-40 | 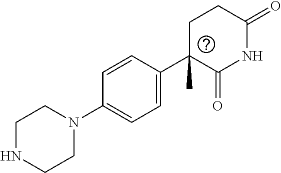 [SMILES: C[C@@]1(c2ccc(N3CCNCC3)cc2)CCC(=O)NC1=O] |

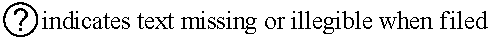

### General Procedure CBM-10

### Example S15. (3R)-3-fluoro-3-[4-(4-piperidyl)phenyl]piperidine-2,6-dione; dihydrochloride (C-46 and C-46i)

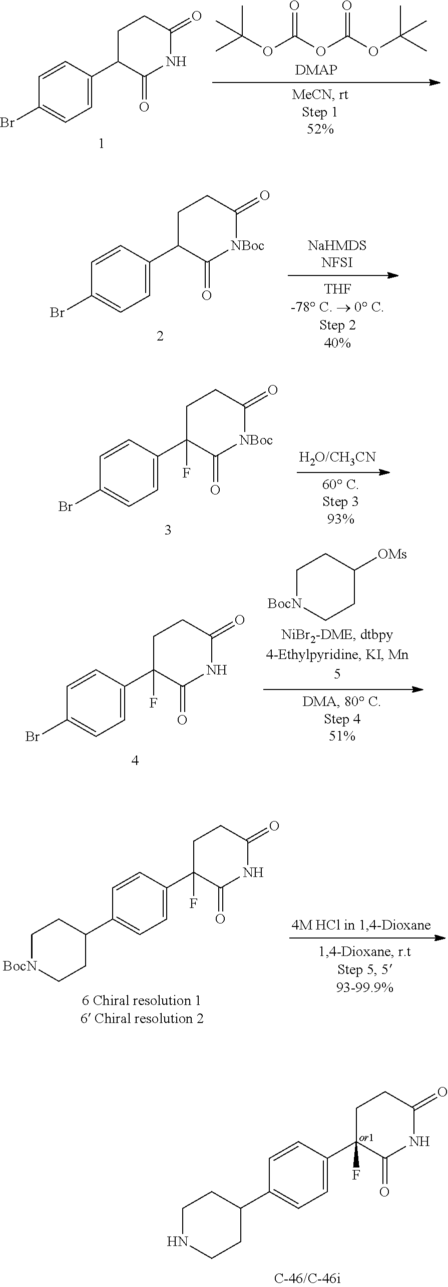

Step 1. Preparation of tert-butyl 3-(4-bromophenyl)-2,6-dioxo-piperidine-1-carboxylate (2): To a suspension of 3-(4-bromophenyl)piperidine-2,6-dione (1) (400 mg, 1.491 mmol) and DMAP (4-Dimethylaminopyridine) (18.2 mg, 0.15 mmol) in MeCN (10 mL) was added Boc₂O (645 mg, 2.985 mmol). The reaction was stirred at RT for 2.5 hrs, and monitored by HPLC/TLC. The reaction mixture was partitioned between DCM and sat. NH₄C₁. Layers were separated and the aqueous phase was extracted with DCM (1*20 mL). The combined organic layer was dried over Na₂SO₄, filtered and concentrated to dryness, which was then purified by flash chromatography SiO2 120 g eluting with EtOAc in heptane (1 CV 0% then 8 CV 0-30% of EtOAc, following by 4 CV 30% of EtOAc). The desired product came out around 25-30% (@ 225 nm). Pure fractions were collected and concentrated to dryness to afford 2 (285 mg, 0.7740 mmol, 52% yield) as an orange semi-solid.

LCMS method 1: Product was not stable in water.

¹H NMR (400 MHz, DMSO-d6) δ ppm 1.49 (s, 9H), 2.01-2.10 (m, 1H), 2.27-2.41 (m, 1H), 2.66-2.76 (m, 1H), 2.84-2.96 (m, 1H), 4.11-4.20 (m, 1H), 7.24 (d, J=8.3 Hz, 2H), 7.58 (d, J=8.3 Hz, 2H).

Step 2. Preparation of tert-butyl 3-(4-bromophenyl)-3-fluoro-2,6-dioxo-piperidine-1-carboxylate (3): To a flame-dried flask was added 1 M NaHMDS in THF (1.94 mL, 1.94 mmol), and freshly distilled THF (5.5 mL) under nitrogen. The solution was cooled down to −78° C. and a solution of tert-butyl 3-(4-bromophenyl)-2,6-dioxo-piperidine-1-carboxylate (2) (650 mg, 1.77 mmol) in THF (4.5 mL) was added dropwise. After the addition, the dry ice bath was exchanged with an ice bath, and the reaction was stirred at 0° C. for 45 min. The reaction was then cooled back down to −78° C. and a solution of NFSI (612.32 mg, 1.94 mmol) in THF (4.5 mL) was added dropwise. After the addition, the dry ice bath was exchanged once again with an ice bath, and the reaction was stirred at 0° C. for 60 min. Upon completion the reaction mixture was quenched with sat. NH₄Cl and then extracted with EtOAc (3 times), and washed with brine (once). The combined organic layers were dried over Na₂SO₄, filtered and concentrated to dryness to afford a crude off-white solid. The crude was purified by normal phase chromatography eluting with EtOAc/Heptane (1 CV 0% of EtOAc, then 12 CV 0-30% of EtOAc, following by 3 CV 30% of EtOAc). The product comes out at ˜27% of EtOAc. Fractions containing product were contaminated with NFSI were collected and concentrated to dryness. This mixture was used in the next step without an additional purification step. 3 (348 mg, 0.712 mmol, 40% yield).

LCMS method 1: 96.6% purity at 215 nm, [M-Boc+H]⁺=286.0.

¹H NMR (400 MHz, DMSO-d6) δ ppm 1.52 (s, 9H), 1.99-2.05 (m, 1H), 2.10-2.25 (m, 1H), 2.53-2.67 (m, 1H), 2.79-3.04 (m, 1H), 7.34-7.49 (m, 2H), 7.63-7.77 (m, 2H).

¹⁹F NMR (377 MHz, DMSO-d6) δ ppm −149.19-−148.99 (m, 1 F).

Step 3. Preparation of 3-(4-bromophenyl)-3-fluoro-piperidine-2,6-dione (4): To a flask was added tert-butyl 3-(4-bromophenyl)-3-fluoro-2,6-dioxo-piperidine-1-carboxylate (3) (345. mg, 0.6 mmol), water (7 mL), and MeCN (7 mL). The reaction mixture was stirred at 60° C. for 1 h. The reaction mixture was concentrated under reduced pressure to give a white solid. To remove the residual amounts of NFSI from the previous step, the crude material was purified by reverse phase chromatography eluting with MeCN/0.1% FA (aq (5-100%). Product came out at around 50-55% of MeCN. Pure fractions were concentrated under reduced pressure to give 4 as a white solid (160 mg, 0.559 mmol, 93% yield).

LCMS method 1: 99.9% purity at 215 nm, [M+2H]⁺=288.0.

¹H NMR (400 MHz, DMSO-d6) δ ppm 2.31-2.44 (m, 2H), 2.69-2.81 (m, 2H), 7.41 (d, J=8.3 Hz, 2H), 7.68 (d, J=8.3 Hz, 2H), 11.44 (br s, 1H).

¹⁹F NMR (377 MHz, DMSO-d6) δ ppm −150.17-−149.95 (m, 1 F).

Step 4. Preparation of tert-butyl 4-[4-[rac-(3R)-3-fluoro-2,6-dioxo-3-piperidyl]phenyl]piperidine-1-carboxylate (6,6′): To a flame-dried sealed tube was added tert-butyl 4-(p-tolylsulfonyloxy)piperidine-1-carboxylate (5) (195.68 mg, 0.55 mmol), 3-(4-bromophenyl)-3-fluoro-piperidine-2,6-dione (4) (90. mg, 0.31 mmol), NiBr₂·DME (9.71 mg, 0.03 mmol), 4,4′-Di-tert-butyl-2,2′-dipyridyl (8.44 mg, 0.03 mmol), KI (54.83 mg, 0.33 mmol), Manganese powder (35.43 mg, 0.64 mmol), DMA (2 mL), and 4-ethylpyridine (0.04 mL, 0.3100 mmol). The reaction mixture was sparged with N₂ for 15 min, then the tube was sealed. The reaction mixture was stirred at 80° C. overnight. Upon completion, the mixture poured in a 0.5 M HCl and extracted with EtOAc (3×). The combined organic phases were washed with brine (1×) and passed through a pad of celite and MgSO₄, and concentrated under reduced pressure. The crude was purified by reverse phase chromatography eluting with MeCN (5-60%)/0.1% F.A(aq.) in water. Product comes out at 60% MeCN. The pure fractions were combined and concentrated under reduced pressure to afford the product as a white solid. The material was sent to chiral SFC to separate the stereoisomers 6,6′ (160 mg for both isomers, 0.559 mmol, 93% yield). After SFC, there were 2 isomers: Chiral resolution 1 named as 6, and Chiral resolution 2 named as 6′. Stereochemistry was arbitrarily assigned.

LCMS method 1: 99.9% purity at 215 nm [M-Boc]₊=291.2 m/z.

¹H NMR (400 MHz, DMSO-d6) δ ppm 1.42 (s, 9H), 1.46-1.58 (m, 2H), 1.72-1.80 (m, 2H), 2.25-2.37 (m, 2H), 2.66-2.93 (m, 5H), 4.00-4.15 (m, 2H), 7.32-7.40 (m, 4H), 11.36 (br s, 1H).

¹⁹F NMR (377 MHz, DMSO-d6) δ ppm −146.87-−146.48 (m, 1 F).

Step 5. Preparation of (3R)-3-fluoro-3-[4-(4-piperidyl)phenyl]piperidine-2,6-dione;dihydrochloride (7): To a flask was added tert-butyl 4-[4-[(3R)-3-fluoro-2,6-dioxo-3-piperidyl]phenyl]piperidine-1-carboxylate (6) (85 mg, 0.22 mmol), 1,4-Dioxane (1.5 mL) and 4 M HCl in dioxane (1.36 mL, 5.44 mmol). The reaction mixture was stirred at RT for 1 h. Upon completion, the reaction was co-evaporated with toluene (2×) and MeCN (2×) to afford C-46 (81 mg, 0.2025 mmol, 93% yield) and used as it for the next step.

LCMS method 1: 90.7% purity at 215 nm, [M+H]⁺=291.2.

Step 5′. Preparation of (3R)-3-fluoro-3-[4-(4-piperidyl)phenyl]piperidine-2,6-dione;dihydrochloride (C-46i): To a solution of tert-butyl 4-[4-[(35)-3-fluoro-2,6-dioxo-3-piperidyl]phenyl]piperidine-1-carboxylate 6′ (88. mg, 0.23 mmol) in 1,4-dioxane (1.13 mL) was added HCl in dioxane (1.41 mL, 5.63 mmol). The reaction was stirred at room temperature for 1 h, which was then concentrated in vacuo and chased with MeCN to afford the crude product C-46i as tan solid (73.5 mg, 0.2249 mmol, 99.9% yield). Used as is in the next step.

LCMS method 1: 99.9% purity at 215 nm, [M+H]⁺=291.2.

CBM intermediates prepared using General Procedure CBM-10 are summarized in Table 11.

TABLE 11
CBM Intermediates Prepared via General Procedure CBM-10

| | Structure |
|---|---|
| C-46 | 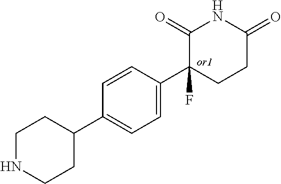 [SMILES: O=C1CC[C@](F)(c2ccc(C3CCNCC3)cc2)C(=O)N1] |
| C-46i | 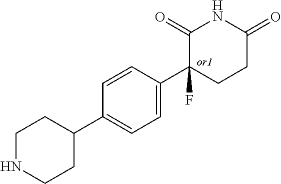 [SMILES: O=C1CC[C@](F)(c2ccc(C3CCNCC3)cc2)C(=O)N1] |

### General Procedure CBM-11 (CBM-11A and CBM-11B)

### General Procedure CBM-11A

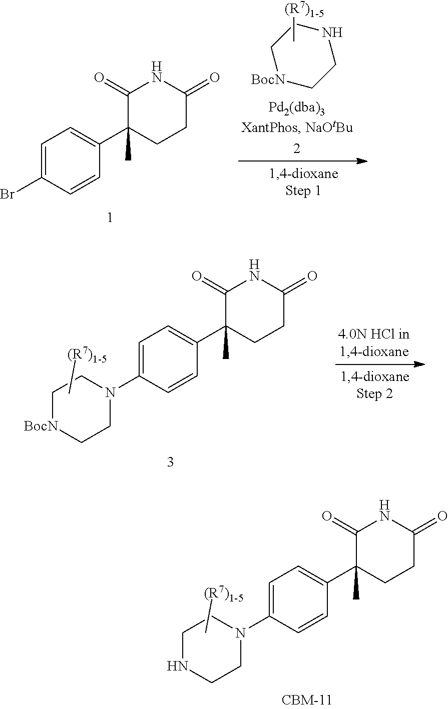

wherein R⁷ is as defined in Formula (I′) or (I).

Step-1′: A suspension of (R)-3-(4-bromophenyl)-3-methylpiperidine-2,6-dione 1 (1.0 g, 3.54 mmol), heterocycle 2 (1.5 eq.) and sodium tert-butoxide (3.0 eq.) in toluene (0.089 M) was purged with nitrogen for 5 min. Pd₂(dba)₃ (0.08 eq.) and Xantphos (0.16 eq.) was added to the reaction mixture under nitrogen before heating to 110° C. After completion of the reaction, RM was poured slowly into 10.0% aqueous solution of acetic acid. RM was then extracted with ethyl acetate. Separated organic layer was washed with water followed by brine solution. Combined organic layer was then dried over sodium sulphate and concentrated under reduced pressure to give the crude product that purified by column chromatography using silica gel to give compound 3.

Step 2′: To a stirred solution of compound 3 (1.0 eq.) in 1,4-dioxane (0.16 M) was added 4.0 M HCl in dioxane (5.0 eq.) at RT. The resulting solution was stirred for 3 h at RT. Reaction mixture was then concentrated under reduced pressure to give crude product CBM-11 that was used in the next step without further purification.

### General Procedure CBM-11B

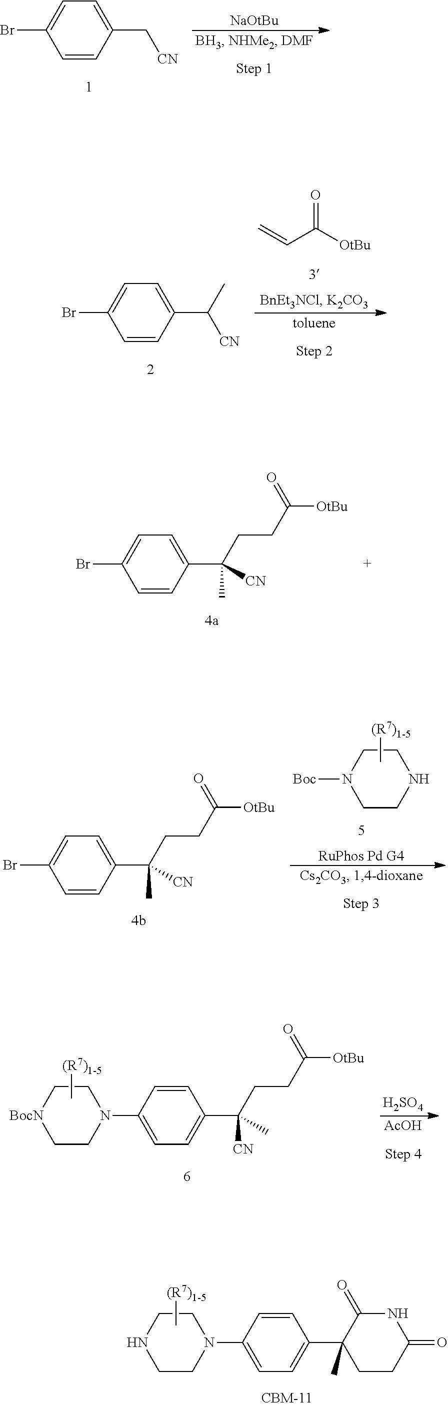

wherein R⁷ is as defined in Formula (I′) or (I).

Step 1. Preparation of 2-(4-bromophenyl)propanenitrile 2: To a solution of 2-(4-bromophenyl)acetonitrile 1 (5.0 g, 25.5 mmol) in DMF (50 mL) was added sodium tert-butoxide (3.68 g, 38.3 mmol) followed by BH₃·NHMe₂ (1.878 mL, 25.5 mmol). The resulting reaction mixture was stirred at 80° C. for 1 h. The reaction mixture was cooled to room temperature, treated with ice-cold water, and extracted with EtOAc (2×150 mL). The combined organics were dried over Na₂SO₄ and concentrated under reduced pressure to obtain the crude compound. The crude compound was purified by column chromatography using silica gel (230-400 mesh) with 6% ethyl acetate/pet ether to obtain 2-(4-bromophenyl)propanenitrile 2 (2.6 g, 12.38 mmol, 48.5% yield) as a pale yellow oil.

Step 2. Preparation of tert-butyl (R)-4-(4-bromophenyl)-4-cyanopentanoate 4a and tert-butyl (S)-4-(4-bromophenyl)-4-cyanopentanoate 4b: To a solution of 2-(4-bromophenyl)propanenitrile 2 (3.8 g, 18.09 mmol) in toluene (40 mL) was added tert-butyl acrylate 3′ (4.64 g, 36.2 mmol), K₂CO₃ (5.00 g, 36.2 mmol), and benzyltriethylammonium chloride (0.824 g, 3.62 mmol). The resulting reaction mixture was heated to 90° C. and stirred for 16 h. The reaction mixture was cooled to room temperature and filtered, and the filtrate was concentrated under reduced pressure to obtain the crude compound. The crude compound was purified by column chromatography using silica gel (230-400 mesh) with 5% ethyl acetate/pet ether to obtain a racemic mixture of tert-butyl 4-(4-bromophenyl)-4-cyanopentanoate (5.5 g, 16.26 mmol, 90% yield) as a pale yellow liquid. 4.5 g of the material was purified by chiral SFC to obtain tert-butyl (R)-4-(4-bromophenyl)-4-cyanopentanoate 4a (1.8 g) and tert-butyl (S)-4-(4-bromophenyl)-4-cyanopentanoate 4b (1.76 g).

SFC Method: CHIRALPAK AD-H, 250×4.6 mm, 5.0 μm. Flow: 3.0 mL/min. Co-Solvent: 10.0% IPA.

Step 3. Preparation of Compound 6: To a stirred solution of tert-butyl (R)-4-(4-bromophenyl)-4-cyanopentanoate 4a (1.0 eq.) in 1,4-dioxane (0.3 M) was added heterocycle 5 (1.1 eq.) followed by Cs₂CO₃ (3.0 eq.). The resulting reaction mixture was degassed for 10 min with nitrogen gas, and then, RuPhos-Pd-G4 (0.02 eq.) was added. The reaction mixture was heated to 90° C. and stirred. The reaction mixture was cooled to room temperature, filtered through celite, and the filtrate was concentrated under reduced pressure to obtain the crude product. The crude product was purified by column chromatography using silica gel to obtain product 6.

Step 4. Preparation of CBM-11: To a stirred solution of 6 (1.0 eq.) in AcOH (0.2 M) was added H₂SO₄ (2.0 eq.). The resulting reaction mixture was heated to 120° C. and stirred. The reaction mixture was concentrated under reduced pressure to obtain product CBM-11.

### Example S16. (R)-3-methyl-3-(4-(piperazin-1-yl) phenyl) piperidine-2,6-dione (C-73)

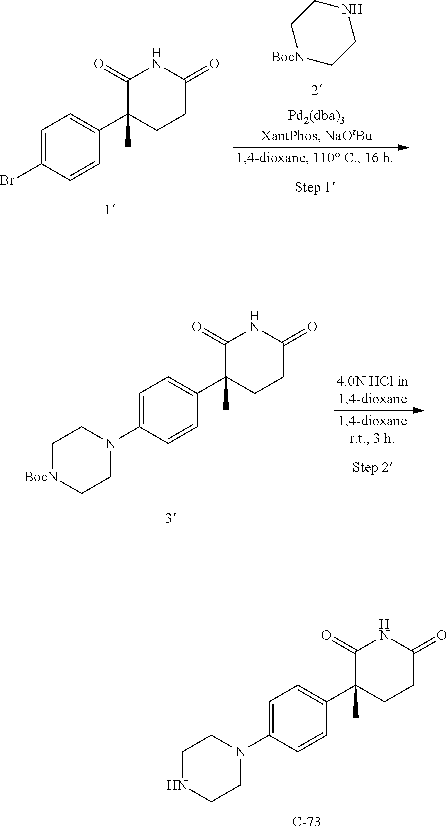

LCMS Method 1. Kinetex XB-C18, 75×3.0 mm, 2.6 Temperature: RT, Flow: 1.0 mL/min, Run Time: 5 minutes, Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 100% Mobile Phase B for 4.0 min. MSD positive.

LCMS Method 2. Aquity UPLC BEH C18, 50×3.0 mm, 1.7 Temperature: RT, Flow: 1.0 mL/min, Run Time: 5 minutes, Mobile Phase Conditions: Mobile Phase-A: 0.1% TFA in H₂O, Mobile Phase-B: 0.1% TFA in ACN, Gradient: Initial 80% Mobile Phase A and 20% Mobile Phase B linear gradient to 100% Mobile Phase B for 3.0 min. MSD positive.

Step-1′. Preparation of tert-butyl (R)-4-(4-(3-methyl-2,6-dioxopiperidin-3-yl) phenyl) piperazine-1-carboxylate 3′: A suspension of (R)-3-(4-bromophenyl)-3-methylpiperidine-2,6-dione 1′ (1.0 g, 3.54 mmol), tert-butyl piperazine-1-carboxylate 2′ (0.996 g, 5.32 mmol) and sodium tert-butoxide (1.022 g, 10.63 mmol) in toluene (10.0 mL, 0.089 M) was purged with nitrogen for 5 min. Pd₂(dba)₃ (0.26 g, 0.284 mmol) and Xantphos (0.328 g, 0.567 mmol) was added to the RM under nitrogen. RM was then heated to 110° C. for 16 h in a sealed vial. After completion of the reaction, RM was poured slowly into 10.0% aqueous solution of acetic acid. RM was then extracted with ethyl acetate (2×25 mL). Separated organic layer was washed with water followed by brine solution (10.0 mL). Combined organic layer was then dried over sodium sulphate and concentrated under reduced pressure to give the crude product. The crude product was purified by column chromatography using silica gel (100-200 mesh) with 30-50% ethyl acetate in pet ether to give pure tert-butyl (R)-4-(4-(3-methyl-2,6-dioxopiperidin-3-yl)phenyl)piperazine-1-carboxylate 3′ (225 mg, 13.75% yield) as pale yellow solid.

LCMS method 1: retention time: 2.181 min, 83.19% purity at 220 nm [M+H]⁺=388.2

Step 2′. Preparation of (R)-3-methyl-3-(4-(piperazin-1-yl) phenyl) piperidine-2,6-dione C-73: To a stirred solution of tert-butyl (R)-4-(4-(3-methyl-2,6-dioxopiperidin-3-yl) phenyl) piperazine-1-carboxylate 3′ (220 mg, 0.476 mmol) in 1,4-dioxane (3.0 mL) was added 4.0 M HCl in dioxane (0.184 mL, 2.38 mmol) at RT. The resulting solution was stirred for 3 h at RT. Reaction mixture was then concentrated under reduced pressure to give crude product (R)-3-methyl-3-(4-(piperazin-1-yl) phenyl) piperidine-2,6-dione C-73 (231 mg) as a pale yellow solid, which was used in the next step without further purification.

LCMS method 2: retention time: 0.456-0.643 min, 66.24% purity at 220 nm, [M+H]⁺=288.2.

### Example S17. (R)-3-methyl-3-(4-((5)-2-methylpiperazin-1-yl)phenyl)piperidine-2,6-dione (C-80)

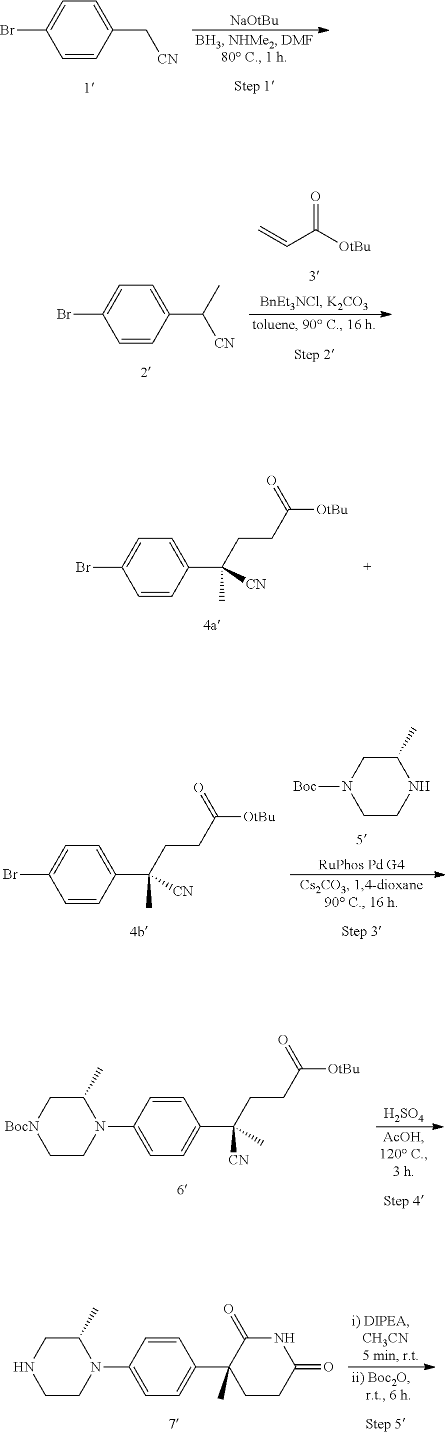

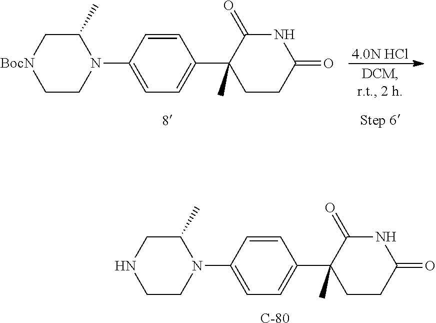

LCMS Method 1. Kinetex XB-C18, 50×4.6 mm, 5.0 Temperature: RT, Flow: 1.0 mL/min, run time: 5.5 min. Mobile Phase Conditions: Mobile Phase-A: 0.1% TFA in H₂O, Mobile Phase-B: 0.1% TFA in ACN, Gradient: Initial 95% Mobile Phase A and 5% Mobile Phase B linear gradient to 95% Mobile Phase B for 2.5 min.

UPLC Method 2. Aquity BEH-C18, 50×2.1 mm, 1.7 μm. Temperature: RT, Flow: 0.7 mL/min, run time: 2.5 min. Mobile Phase Conditions: Mobile Phase-A: 0.1% TFA in H₂O, Mobile Phase-B: 0.1% TFA in ACN, Gradient: Initial 95% Mobile Phase A and 5% Mobile Phase B linear gradient to 98% Mobile Phase B for 1.5 min.

LCMS Method 3. Kinetex XB-C18, 75×3.0 mm, 2.6 μm. Temperature: RT, Flow: 1.0 mL/min, run time: 5.0 min. Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 98% Mobile Phase A and 2% Mobile Phase B linear gradient to 100% Mobile Phase B for 4 min.

Step 1′. Preparation of 2-(4-bromophenyl)propanenitrile 2′: To a solution of 2-(4-bromophenyl)acetonitrile 1′ (5.0 g, 25.5 mmol) in DMF (50 mL) was added sodium tert-butoxide (3.68 g, 38.3 mmol) followed by BH₃·NHMe₂ (1.878 mL, 25.5 mmol). The resulting reaction mixture was stirred at 80° C. for 1 h. The reaction mixture was cooled to room temperature, treated with ice-cold water, and extracted with EtOAc (2×150 mL). The combined organics were dried over Na₂SO₄ and concentrated under reduced pressure to obtain the crude compound. The crude compound was purified by column chromatography using silica gel (230-400 mesh) with 6% ethyl acetate/pet ether to obtain 2-(4-bromophenyl)propanenitrile 2′ (2.6 g, 12.38 mmol, 48.5% yield) as a pale yellow oil.

¹H NMR (400 MHz, CDCl₃): δ ppm 1.65 (d, J=7.2 Hz, 3H), 3.89 (q, J=7.2 Hz, 1H), 7.24-7.27 (m, 2H), 7.53-7.56 (m, 2H).

Step 2′. Preparation of tert-butyl (R)-4-(4-bromophenyl)-4-cyanopentanoate 4a″ and tert-butyl (S)-4-(4-bromophenyl)-4-cyanopentanoate 4b′: To a solution of 2-(4-bromophenyl)propanenitrile 2′ (3.8 g, 18.09 mmol) in toluene (40 mL) was added tert-butyl acrylate 3′ (4.64 g, 36.2 mmol), K₂CO₃ (5.00 g, 36.2 mmol), and benzyltriethylammonium chloride (0.824 g, 3.62 mmol). The resulting reaction mixture was heated to 90° C. and stirred for 16 h. The reaction mixture was cooled to room temperature and filtered, and the filtrate was concentrated under reduced pressure to obtain the crude compound. The crude compound was purified by column chromatography using silica gel (230-400 mesh) with 5% ethyl acetate/pet ether to obtain a racemic mixture of tert-butyl 4-(4-bromophenyl)-4-cyanopentanoate (5.5 g, 16.26 mmol, 90% yield) as a pale yellow liquid. 4.5 g of the material was purified by chiral SFC to obtain tert-butyl (R)-4-(4-bromophenyl)-4-cyanopentanoate 4a′ (1.8 g) and tert-butyl (S)-4-(4-bromophenyl)-4-cyanopentanoate 4b′ (1.76 g).

SFC Method: CHIRALPAK AD-H, 250×4.6 mm, 5.0 μm. Flow: 3.0 mL/min. Co-Solvent: 10.0% IPA.

Characterization of tert-butyl (R)-4-(4-bromophenyl)-4-cyanopentanoate 4a′: rt 2.25; ee 97%; ¹H NMR (400 MHz, CDCl₃): δ ppm 1.42 (s, 9H), 1.74 (s, 3H), 2.10-2.27 (m, 3H), 2.39-2.45 (m, 1H), 7.32-7.36 (m, 2H), 7.53-7.56 (m, 2H).

Characterization of tert-butyl (S)-4-(4-bromophenyl)-4-cyanopentanoate 4b′: rt 1.941; ee 99.9%; ¹H NMR (400 MHz, CDCl₃): δ ppm 1.43 (s, 9H), 1.74 (s, 3H), 2.09-2.28 (m, 3H), 2.39-2.44 (m, 1H), 7.32-7.36 (m, 2H), 7.53-7.57 (m, 2H).

Step 3′. Preparation of tert-butyl (S)-4-(4-((R)-5-(tert-butoxy)-2-cyano-5-oxopentan-2-yl)phenyl)-3-methylpiperazine-1-carboxylate 6′: To a stirred solution of tert-butyl (R)-4-(4-bromophenyl)-4-cyanopentanoate 4a′ (2.0 g, 5.91 mmol, 1.0 eq.) in 1,4-dioxane (20.0 mL) was added tert-butyl (S)-3-methylpiperazine-1-carboxylate 5′ (1.303 g, 6.50 mmol, 1.1 eq.) followed by Cs₂CO₃ (5.78 g, 17.74 mmol, 3.0 eq.). The resulting reaction mixture was degassed for 10 min with nitrogen gas, and then, RuPhos-Pd-G4 (0.101 g, 0.118 mmol, 0.02 eq.) was added. The reaction mixture was heated to 90° C. and stirred for 16 h. The reaction mixture was cooled to room temperature, filtered through celite, and the filtrate was concentrated under reduced pressure to obtain the crude product. The crude product was purified by column chromatography using silica gel (230-400 mesh) with 25% ethyl acetate/pet ether to obtain tert-butyl (S)-4-(4-((R)-5-(tert-butoxy)-2-cyano-5-oxopentan-2-yl)phenyl)-3-methylpiperazine-1-carboxylate 6′ (2.2 g, 78% yield) as a colorless gummy liquid.

LCMS method 1: retention time: 2.859 min, 96.2% purity at 220 nm, [M+H]⁺=458.3

Step 4′. Preparation of (R)-3-methyl-3-(4-((S)-2-methylpiperazin-1-yl)phenyl)piperidine-2,6-dione 7′: To a stirred solution of tert-butyl (S)-4-(4-((R)-5-(tert-butoxy)-2-cyano-5-oxopentan-2-yl)phenyl)-3-methylpiperazine-1-carboxylate 6′ (2.0 g, 4.37 mmol, 1.0 eq.) in AcOH (20.0 mL) was added H₂SO₄ (0.466 mL, 8.74 mmol, 2.0 eq.). The resulting reaction mixture was heated to 120° C. and stirred for 3 h. The reaction mixture was concentrated under reduced pressure to obtain (R)-3-methyl-3-(4-((S)-2-methylpiperazin-1-yl)phenyl)piperidine-2,6-dione 7′ (3.5 g) as a brown color gum, which was used in the next step without further purification.

UPLC method 2: retention time: 0.809 min, [M+H]⁺=302.2

Step 5′. Preparation of tert-butyl (S)-3-methyl-4-(4-(((R)-3-methyl-2,6-dioxopiperidin-3-yl)phenyl)piperazine-1-carboxylate 8′: To a stirred solution of (R)-3-methyl-3-(4-((S)-2-methylpiperazin-1-yl)phenyl)piperidine-2,6-dione 7′ (3.5 g, 4.18 mmol, 1.0 eq.) in acetonitrile (20 mL) at 0° C. was added DIPEA (5.84 mL, 33.4 mmol, 8.0 eq.) and stirred for 5 min. Then Di-tert-butyl decarbonate (1.941 mL, 8.36 mmol, 2.0 eq.) was added to the reaction mixture, and the resulting reaction mixture was stirred at RT for 6 h. Then, the reaction mixture was concentrated under reduced pressure to obtain the crude product. The crude product was purified by column chromatography using silica gel (230-400 mesh) with 40% ethyl acetate/pet ether to obtain tert-butyl tert-butyl (S)-3-methyl-4-(4-((R)-3-methyl-2,6-dioxopiperidin-3-yl)phenyl)piperazine-1-carboxylate 8′ (1.45 g, 84% yield) as an off-white solid.

LCMS method 1: retention time: 2.150 min, 97.3% purity at 220 nm, [M+H]⁺=402.3.

Step 6′. Preparation of (R)-3-methyl-3-(4-((5)-2-methylpiperazin-1-yl)phenyl)piperidine-2,6-dione C-80: To a stirred solution of tert-butyl (S)-3-methyl-4-(4-((R)-3-methyl-2,6-dioxopiperidin-3-yl)phenyl)piperazine-1-carboxylate 8′ (1.45 g, 3.61 mmol, 1.0 eq.) in DCM (4 mL, 0.903 M) at 0° C. was added 4.0 N HCl in 1,4-dioxane (4.51 mL, 18.06 mmol, 5.0 eq.). The resulting reaction mixture was stirred at RT for 2 h. The reaction mixture was concentrated under reduced pressure to obtain crude (R)-3-methyl-3-(4-((S)-2-methylpiperazin-1-yl)phenyl)piperidine-2,6-dione, 2HCl C-80 (1.2 g), which was used in the next step without further purification.

LCMS method 3: retention time: 1.565 min, 95.17% purity at 220 nm, [M+H]⁺=302.1.

CBM intermediates prepared using General Procedure CBM-11 are summarized in Table 12.

TABLE 12
CBM Intermediates Prepared via General Procedure CBM-11.

| | Structure | Heterocycle |
|---|---|---|
| C-73 | 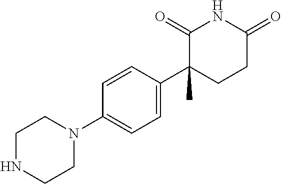 [SMILES: C[C@]1(c2ccc(N3CCNCC3)cc2)CCC(=O)NC1=O] | 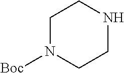 [SMILES: CC(C)(C)OC(=O)N1CCNCC1] |
| C-73i | 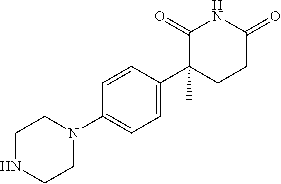 [SMILES: C[C@@]1(c2ccc(N3CCNCC3)cc2)CCC(=O)NC1=O] | 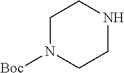 [SMILES: CC(C)(C)OC(=O)N1CCNCC1] |
| | Using the inverse epimer | |
| C-75 | 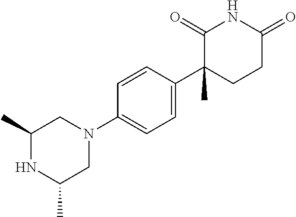 [SMILES: C[C@H]1CN(c2ccc([C@@]3(C)CCC(=O)NC3=O)cc2)C[C@H](C)N1] | 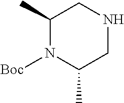 |
| C-78 | 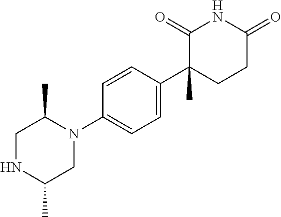 [SMILES: C[C@@H]1CN[C@@H](C)CN1c1ccc([C@@]2(C)CCC(=O)NC2=O)cc1] | 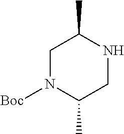 |
| C-79 | 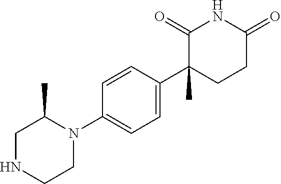 [SMILES: C[C@@H]1CNCCN1c1ccc([C@@]2(C)CCC(=O)NC2=O)cc1] | 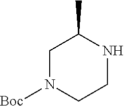 |
| C-80 | 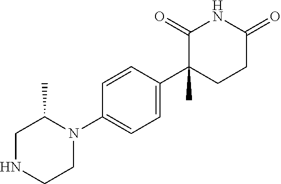 [SMILES: C[C@H]1CNCCN1c1ccc([C@@]2(C)CCC(=O)NC2=O)cc1] | 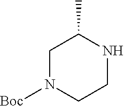 |
| C-81 | 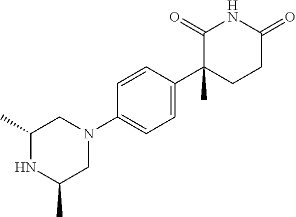 [SMILES: C[C@@H]1CN(c2ccc([C@@]3(C)CCC(=O)NC3=O)cc2)C[C@@H](C)N1] | 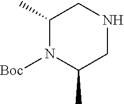 |
| C-82 | 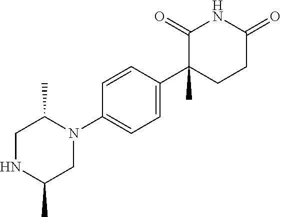 [SMILES: C[C@@H]1CN(c2ccc([C@@]3(C)CCC(=O)NC3=O)cc2)[C@@H](C)CN1] | 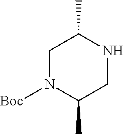 |

### General Procedure CBM-12

### Example S18. (R)-3-methyl-3-(4-(piperidin-4-yl)phenyl)piperidine-2,6-dione hydrochloride (C-76)

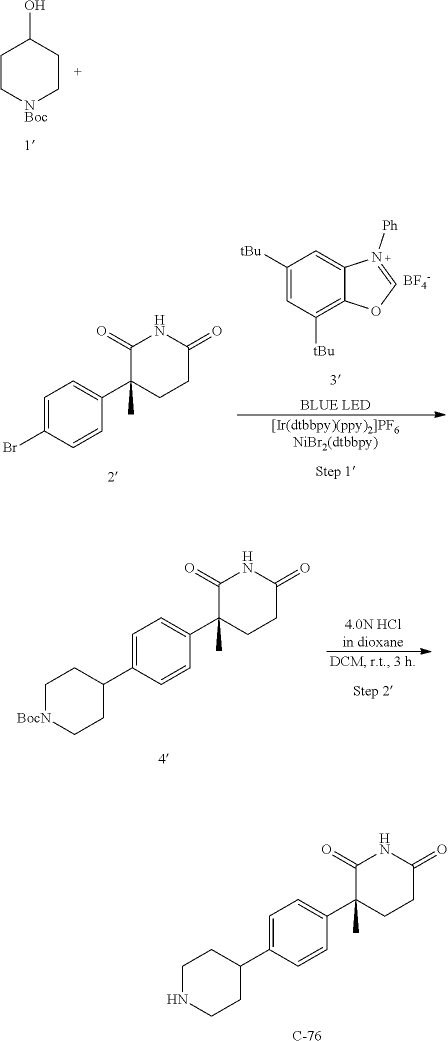

LCMS Method 1. Kinetex XB-C 18, 75×3.0 mm, 2.6 Temperature: RT, Flow: 1.0 mL/min, Run Time: 5 minutes, Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 100% Mobile Phase B for 4.0 min. MSD positive.

Step 1′. Synthesis of tert-butyl (R)-4-(4-(3-methyl-2,6-dioxopiperidin-3-yl)phenyl)piperidine-1-carboxylate 4′: To a mixture of tert-butyl 4-hydroxypiperidine-1-carboxylate 1′ (174.7 mg, 0.868 mmol) and 5,7-di-tert-butyl-3-phenylbenzo[d]oxazol-3-ium·BF₄ 3′ (314 mg, 0.794 mmol) in 20 mL vial was added anhydrous tert-butyl methyl ether (4.0 mL) and the reaction mixture was stirred at room temperature for 5 min. Then, pyridine (0.064 mL, mmol) in 1.0 mL of anhydrous tert-butyl methyl ether was added dropwise at room temperature. The resulting solution was stirred at room temperature for 10 min. A white solid precipitated out during this time. Another 40.0 mL vial was charged with [Ir(dtbbpy)(ppy)₂]PF₆ (6.80 mg, 7.44 μmol), NiBr₂(dtbbpy) (12.08 mg, 0.025 mmol), quinuclidine (97 mg, 0.868 mmol), (R)-3-(4-bromophenyl)-3-methylpiperidine-2,6-dione 2′ (140 mg, 0.496 mmol) and phthalimide (16.79 mg, 0.114 mmol). N,N-Dimethylacetamide (5.0 mL) was added to this vial under nitrogen atmosphere.

The tert-butyl methyl ether suspension was transferred to a syringe. Then a syringe filter was installed on the syringe, and the tert-butyl methyl ether solution was injected into the dimethylacetamide solution. The reaction was degassed with nitrogen for 15 min and closed with a screw cap. The reaction mixture's vial was irradiated under blue LED light for 2 h under constant stirring. The reaction mixture was diluted with water (10 mL) and extracted with DCM (20 mL). The organic layer was concentrated under reduced pressure and purified by column chromatography on silica gel with 30% ethyl acetate/pet ether to afford compound tert-butyl (R)-4-(4-(3-methyl-2,6-dioxopiperidin-3-yl)phenyl)piperidine-1-carboxylate 4′ (90 mg, 47.0% yield) as an off-white solid.

LCMS method 1: retention time 2.82 min, 99.09% purity at 220 nm, [M+H-Boc]⁺287.2

Step 2′. Preparation of (R)-3-methyl-3-(4-(piperidin-4-yl)phenyl)piperidine-2,6-dione hydrochloride C-76: To a stirred solution of tert-butyl (R)-4-(4-(3-methyl-2,6-dioxopiperidin-3-yl)phenyl)piperidine-1-carboxylate 4′ (140 mg, 0.362 mmol) in DCM (1.5 mL) was added 4.0 N HCl in dioxane (1.5 mL) at RT. The resulting solution was stirred for 3 h at RT. Reaction mixture was then concentrated under reduced pressure to give crude product (R)-3-methyl-3-(4-(piperidin-4-yl)phenyl)piperidine-2,6-dione hydrochloride C-76 (120 mg), which was used in the next step without further purification.

LCMS method 1: retention time 1.647 min, 98.14% purity at 220 nm, [M+H]⁺=287.2.

CBM intermediates prepared using General Procedure CBM-12 are summarized in Table 13.

TABLE 13
CBM Intermediates Prepared via General Procedure CBM-12.

| | Structure |
|---|---|
| C-76 | 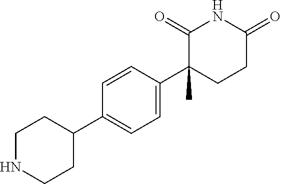 [SMILES: C[C@]1(c2ccc(C3CCNCC3)cc2)CCC(=O)NC1=O] |

### Synthesis of the TBM Molecule

The numbering of the intermediate compounds referred to in this section is limited to each section only. For instance, intermediate 3 in General Procedure TBM-1 and intermediate 3 in General Procedure TBM-3 are not the same compounds as they are from different sections.

### General Procedure TBM-1

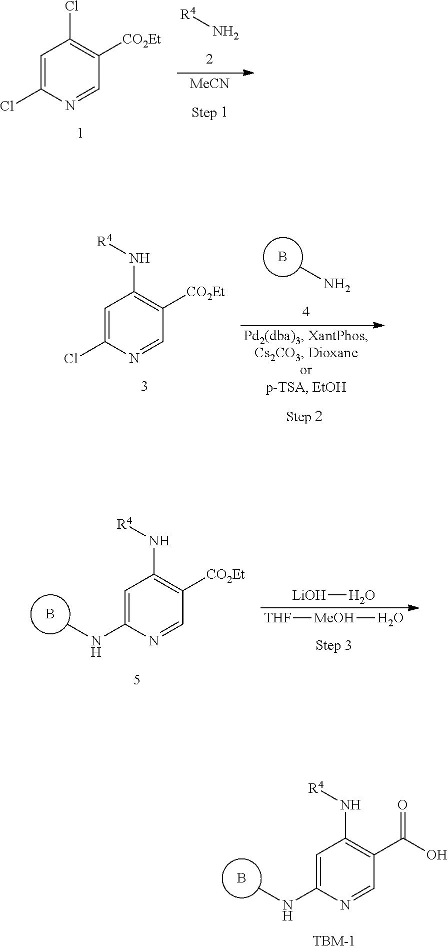

wherein Ring B is heteroaryl; R⁴ is C₃-C₆ cycloalkyl; and R⁰ is as defined in Formula (I′) or (I).

Step 1: To a solution of ethyl 4,6-dichloropyridine-3-carboxylate 1 (1 eq.) in MeCN (0.45 M) was added amine 2 (3 eq.). The reaction was stirred at temperature. Upon completion by LCMS, the reaction mixture was cooled to room temperature and reaction was purified to afford 3.

Step 2 (option 1): To reaction vessel was added ethyl 6-chloro-4-(amino)pyridine-3-carboxylate 3 (1 eq.), heterocyclic anline 4 (1.2 eq.), Xantphos (0.05 eq.), Cs₂CO₃ (1.4 eq.) and Pd₂(dba)₃ (0.05 eq.) in 1,4-Dioxane (0.2 M). The solution was sparged with nitrogen for 5 min, the tube was sealed and stirred at 150° C. Upon LCMS indication of conversion, the reaction mixture cooled to room temperature mixture prior to purification by reverse phase column chromatography to afford product 5.

Step 2 (option 2): To reaction vessel was added ethyl 6-chloro-4-(amino)pyridine-3-carboxylate 3 (1 eq.), heterocyclic anline 4 (1.5 eq.), p-toluenesulfonic acid monohydrate (0.3 eq.) and ethanol (3 mL). The tube was purged with nitrogen, sealed and stirred at 105° C. Upon LCMS indication of conversion, EtOH was removed under vacuum and the residue purified by reverse phase flash chromatography to afford product 5.

Step 3: In a round-bottom flask were added ethyl 4-(amino)-6-[heterocyclic]pyridine-3-carboxylate 5 (1 eq.), lithium hydroxide monohydrate (5 eq.), THF (1 mL), MeOH (1 mL), and water (1 mL). The reaction was stirred at 60° C. Upon LCMS indication of conversion, volatiles were removed under vacuum and the residue taken up with water (5 mL). A 6 M aqueous solution of HCl was added to reach pH 3. The precipitate was filtered off and dried under vacuum to afford product TBM-1.

### Example S19. 6-(1,3-benzothiazol-6-ylamino)-4-(cyclopentylamino)pyridine-3-carboxylic acid (A-1)

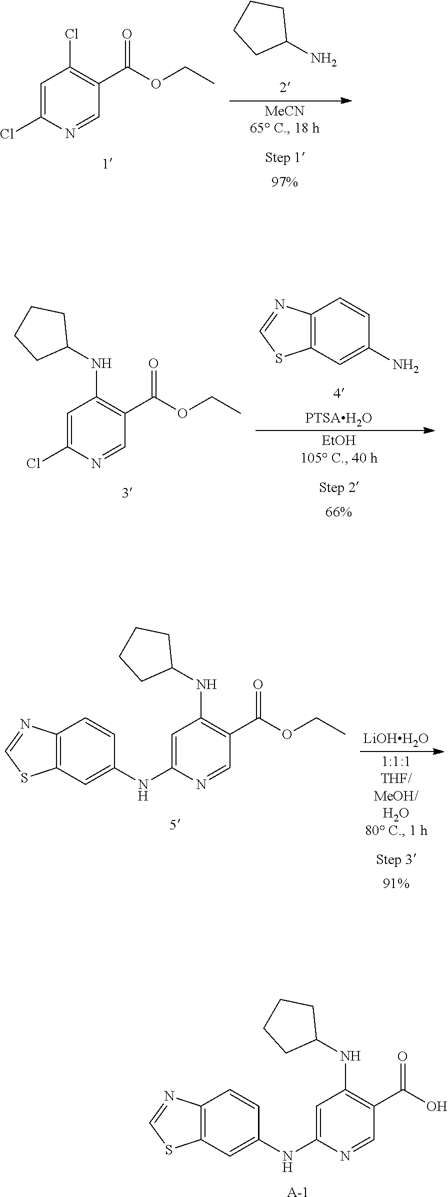

Step 1′. Preparation of ethyl 6-chloro-4-(cyclopentylamino)pyridine-3-carboxylate (3′): Ethyl 4,6-dichloropyridine-3-carboxylate (1′) (10 g, 45.4 mmol, 1.0 equiv) was added to a a solution of cyclopentanamine (2′) (17.9 mL, 182 mmol, 4.0 equiv) in MeCN (100 mL), then the reaction mixture was stirred at 65° C. overnight. The reaction mixture was cooled to room temperature and water (400 mL) was added. The suspension was sonicated and then stirred at 0° C. for 1 hour. The solid was filtered, rinsed with water, and dried under high vacuum to yield 3′ (11.9 g, 44.1 mmol, 97% yield) as a brown solid.

LCMS method 1: 96.5% purity at 215 nm, [M+H]⁺=269.2.

¹H NMR (400 MHz, DMSO-d6) δ ppm 1.30 (t, J=7.1 Hz, 3H), 1.40-1.51 (m, 2H), 1.56-1.73 (m, 4H), 2.02 (dq, J=12.2, 6.0 Hz, 2H), 3.97 (sxt, J=6.3 Hz, 1H), 4.29 (q, J=7.1 Hz, 2H), 6.82 (s, 1H), 8.08 (br d, J=7.0 Hz, 1H), 8.52 (s, 1H).

Step 2′. Preparation of ethyl 6-(1,3-benzothiazol-6-ylamino)-4-(cyclopentylamino)pyridine-3-carboxylate (5′): 1,3-Benzothiazol-6-amine (4′) (4.19 g, 27.9 mmol, 1.5 equiv), ethyl 6-chloro-4-(cyclopentylamino)pyridine-3-carboxylate (3′) (5 g, 18.6 mmol, 1.0 equiv) and PTSA·H₂O (1.42 g, 7.44 mmol, 0.4 equiv) were mixed with ethanol (37.2 mL) in a sealed tube and the reaction mixture was stirred at 105° C. for 40 hours. The organic solvents were evaporated under reduced pressure, then the crude mixture was taken up in the minimum amount of EtOAc and basified with saturated NaHCO₃(aq) until the product crashed out. The precipitate was filtered and triturated with 1:1 MeCN/H₂O, then it was filtered again and rinsed with water to yield 5′ (4.72 g, 12.34 mmol, 66% yield) as a green solid.

LCMS method 1: 98.5% purity at 215 nm, [M+H]⁺=383.2.

¹H NMR (400 MHz, DMSO-d6) δ ppm 1.29 (t, J=7.1 Hz, 3H), 1.49 (br dd, J=11.9, 5.9 Hz, 2H), 1.56-1.78 (m, 4H), 1.97-2.05 (m, 2H), 3.39-3.43 (m, 1H), 3.78 (dq, J=12.0, 6.0 Hz, 1H), 4.23 (q, J=7.1 Hz, 2H), 6.06 (s, 1H), 7.56 (dd, J=8.8, 1.7 Hz, 1H), 7.78 (br d, J=6.2 Hz, 1H), 7.95 (d, J=8.8 Hz, 1H), 8.57 (s, 1H), 8.62 (d, J=1.3 Hz, 1H), 9.16 (s, 1H), 9.42 (s, 1H).

Step 3. Preparation of 6-(1,3-benzothiazol-6-ylamino)-4-(cyclopentylamino)pyridine-3-carboxylic acid (A-1): A solution of LiOH·H₂O (2.59 g, 61.7 mmol, 5.0 equiv) in water (15 mL) was added to a solution of ethyl 6-(1,3-benzothiazol-6-ylamino)-4-(cyclopentylamino)pyridine-3-carboxylate (5′) (4.72 g, 12.3 mmol, 1.0 equiv) in THF (15 mL) and methanol (15 mL), then the reaction mixture was stirred at 80° C. for 1 hour. The organic solvents were evaporated under reduced pressure, then the crude mixture was diluted with water (75 mL) and acidified with 3.0 M HCl(aq) to pH=1. The precipitate was filtered, rinsed thoroughly with water and dried under high vacuum to yield A-1 (4.03 g, 11.2 mmol, 91% yield) as a tan solid.

LCMS method 2: 98.7% purity at 215 nm, [M+H]⁺=355.1.

¹H NMR (400 MHz, DMSO-d6) δ ppm 1.41-1.55 (m, 2H), 1.55-1.72 (m, 4H), 1.92-2.10 (m, 2H), 3.75-3.87 (m, 1H), 6.11 (s, 1H), 7.55 (br d, J=8.2 Hz, 1H), 8.05 (br d, J=8.8 Hz, 1H), 8.38 (br d, J=3.3 Hz, 1H), 8.43 (br s, 2H), 9.27 (s, 1H), 10.02 (br s, 1H), 12.95 (br s, 1H).

### Example S20. 6-(2-chloro-4-cyano-anilino)-4-(isopropylamino)pyridine-3-carboxylic acid (A-53)

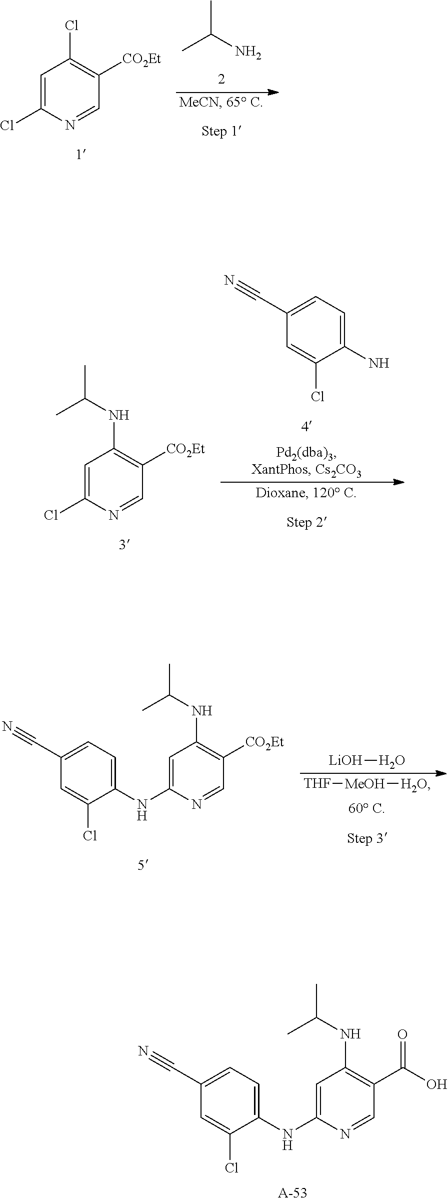

LCMS Method 1. Column: Luna C18 (2) 50×3 mm, 3 um. Temperature: 45° C., Flow: 1.5 mL/min, run time: 2.5 min. Mobile phase conditions: Initial 95% H₂O 0.1% FA/5% MeCN 0.1% FA, linear gradient to 95 MeCN 0.1% FA over 1.3 min then hold for 1.2 minute at 95% MeCN 0.1% FA. MSD: ESI Positive

LCMS Method 3. Kinetex Polar C18 2.6 um, 50×3.0 mm. Temperature: 45° C., Flow: 1.2 mL/min, Run time: 3 min. Mobile phase conditions: Initial 95% H₂O+0.1% FA/5% MeCN+0.1% FA then linear gradient to 95% MeCN for 1.5 min then hold for 1.5 min at 95% MeCN. MSD: Positive

Step 1″. Preparation of ethyl 6-chloro-4-(isopropylamino)pyridine-3-carboxylate (3″): To a solution of ethyl 4,6-dichloropyridine-3-carboxylate 1″ (5 g, 22.72 mmol, 1 eq.) in MeCN (50 mL, 0.45 M) was added propan-2-amine 2″ (5.86 mL, 68.17 mmol, 3 eq.). The reaction was stirred at 65° C. After an overnight period, LCMS showed complete conversion into compound 3″. The reaction mixture was cooled to room temperature, water was added, and the solution was sonicated for 1 minute and stirred for 1 hour at 0° C. The precipitate was filtered and dried under vacuum, affording 3″ (5.35 g, 97% yield) as a light pink solid.

LCMS method 1: 99.9% purity at 215 nm [M+H]⁺=243.2.

¹H NMR (400 MHz, chloroform-d) δ ppm 1.29 (d, J=6.4 Hz, 6H), 1.40 (t, J=7.1 Hz, 3H), 3.69 (dq, J=13.3, 6.7 Hz, 1H), 4.34 (q, J=7.3 Hz, 2H), 6.55 (s, 1H), 8.11 (br s, 1H), 8.67 (s, 1H).

Step 2′. Preparation of ethyl 6-(2-chloro-4-cyano-anilino)-4-(isopropylamino)pyridine-3-carboxylate (5″): To a flame dried sealed tube was added ethyl 6-chloro-4-(isopropylamino)pyridine-3-carboxylate 3′ (100 mg, 0.41 mmol, 1 eq.), 4-amino-3-chloro-benzonitrile 4″ (75.44 mg, 0.49 mmol, 1.2 eq.), Xantphos (11.92 mg, 0.02 mmol, 0.05 eq.), Cs₂CO₃ (187.9 mg, 0.58 mmol, 1.4 eq.) and Pd₂(dba)₃ (18.86 mg, 0.02 mmol, 0.05 eq.) in 1,4-Dioxane (2 mL, 0.2 M). The solution was sparged with nitrogen for 5 min, the tube was sealed and stirred at 150° C. After an overnight period, LCMS showed 77% conversion into compound 5′. The reaction was cooled to room temperature and the mixture was passed over celite and the pad was washed with EtOAc. The solvent was evaporated under reduced pressure and the residue was purified by reverse phase FC purification (50 g C18 column, liquid deposit (DMSO), 5% MeOH/0.1% HCOOH over 4 CV, then 5 to 95% MeOH/0.1% HCOOH over 15 CV, product came out around 70% MeOH). The pure tubes were combined and concentrated to dryness, affording 5″ (105 mg, 55% yield) as a red oil.

LCMS method 3: 77.5% purity at 215 nm [M+H]⁺=359.1.

¹H NMR (400 MHz, DMSO-d6) δ ppm 1.24 (d, J=6.4 Hz, 6H), 1.30 (t, J=6.8 Hz, 3H), 3.61-3.71 (m, 1H), 4.25 (q, J=7.1 Hz, 2H), 6.58 (s, 1H), 7.66-7.72 (m, 1H), 7.74-7.78 (m, 1H), 8.00 (d, J=1.7 Hz, 1H), 8.43 (d, J=8.8 Hz, 1H), 8.54 (s, 1H), 8.84 (s, 1H)

Step 3′. Preparation of 6-(2-chloro-4-cyano-anilino)-4-(isopropylamino)pyridine-3-carboxylic acid (A-53): To a round bottom flask was added ethyl 6-(2-chloro-4-cyano-anilino)-4-(isopropylamino)pyridine-3-carboxylate 5′ (105 mg, 0.29 mmol, 1 eq.) and LiOH·H₂O (61.39 mg, 1.46 mmol, 5 equiv) in H₂O:THF:MeOH (0.49 mL: 0.49 mL; 0.49 mL, 0.2 M), then the reaction mixture was stirred at room temperature. After 1 hour, LCMS showed complete conversion into compound 6″. The organic solvents were evaporated under reduced pressure, then the crude mixture was purified by reverse phase FC purification (50 g C18 column, liquid deposit (DMSO), 5% MeCN/0.1% HCOOH over 4 CV, then 5 to 95% MeCN/0.1% HCOOH over 15 CV, product came out around 35% MeCN). The pure tubes were combined and concentrated to dryness, affording A-53 (40 mg, 38% yield) as a white solid.

LCMS method 3: 91.5% purity at 215 nm, [M+H]⁺=331.1.

¹H NMR (400 MHz, DMSO-d6) δ ppm 1.23 (d, J=6.4 Hz, 6H), 3.60-3.70 (m, 1H), 6.57 (s, 1H), 7.69 (dd, J=8.7, 2.1 Hz, 1H), 7.94-8.02 (m, 2H), 8.45 (d, J=8.8 Hz, 1H), 8.50 (s, 1H), 8.77 (s, 1H), 12.70 (br s, 1H).

### Example S21. 4-(cyclopropylamino)-6-[(2-oxo-3H-1,3-benzothiazol-6-yl)amino]pyridine-3-carboxylic acid (A-59)

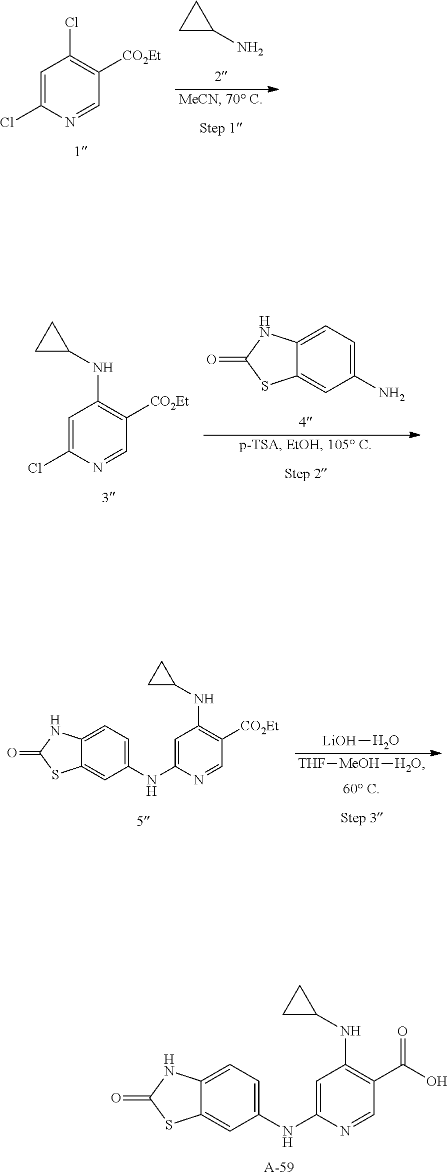

LCMS Method 1. Column: Luna C18 (2) 50×3 mm, 3 um. Temperature: 45° C., Flow: 1.5 mL/min, run time: 2.5 min. Mobile phase conditions: Initial 95% H₂O 0.1% FA/5% MeCN 0.1% FA, linear gradient to 95% MeCN 0.1% FA over 1.3 min then hold for 1.2 minute at 95 MeCN 0.1% FA. MSD: ESI Positive.

Step 1′. Preparation of ethyl 6-chloro-4-(cyclopropylamino)pyridine-3-carboxylate (3′″): A 250 mL round bottom flask was loaded with tert-butyl 4,6-dichloropyridine-3-carboxylate 1′″ (1.5 g, 6.82 mmol, 1 eq.), MeCN (50 mL) and cyclopropanamine 2′ (1.26 mL, 20.45 mmol, 3 eq.) and the mixture was stirred at 70° C. After 18 h, LCMS showed complete conversion. Volatiles were removed under vacuum and the residue was purified by normal phase flash chromatography (40 g silica column; elution: 0% EtOAc/Heptane for 3 CVs, 0% to 20% EtOAc/Heptane over 13 CVs). Fractions were collected and concentrated to give 3′ (1.5 g, 6.232 mmol, 92% yield) as a white solid.

LCMS method 1: 99.9% purity at 215 nm, [M+H]⁺=241.2.

¹H NMR (400 MHz, CDCl₃) δ ppm 0.61-0.66 (m, 2H), 0.88-0.94 (m, 2H), 1.39-1.42 (t, J=7.2 Hz, 3H), 2.47-2.55 (m, 1H), 4.35 (q, J=7.1 Hz, 2H), 6.98 (s, 1H), 8.23 (br s, 1H), 8.68 (s, 1H).

Step 2′. Preparation of ethyl 4-(cyclopropylamino)-6-[(2-oxo-3H-1,3-benzothiazol-6-yl)amino]pyridine-3-carboxylate (5″): To a sealed tube were added ethyl 6-chloro-4-(cyclopropylamino)pyridine-3-carboxylate 3′ (100 mg, 0.42 mmol, 1 eq.), 6-aminobenzo[d]thiazol-2(3H)-one 4″ (104 mg, 0.62 mmol, 1.5 eq.), p-toluenesulfonic acid monohydrate (24 mg, 0.12 mmol, 0.3 eq.) and ethanol (3 mL). The tube was purged with nitrogen, sealed and stirred at 105° C. After 2 days, LCMS showed 70% conversion. EtOH was removed under vacuum and the residue purified by reverse phase flash chromatography (liquid deposit (DMSO), elution: 5% MeCN/water over 10 CV, 5%-40% MeCN/water over 10 CV and 40%-100 MeCN/water over 10 CV. Product came out of the column about 98% of MeCN. The fractions were combined and concentrated to give 5′″ (100 mg, 0.2457 mmol, 60% yield) as an off-white solid.

LCMS method 1: 99.9% purity at 215 nm, [M+H]⁺=371.1.

¹H NMR (400 MHz, DMSO-d6) δ ppm 0.53-0.61 (m, 2H), 0.76-0.84 (m, 2H), 1.29 (t, J=7.1 Hz, 3H), 4.24 (q, J=7.1 Hz, 2H), 6.33 (s, 1H), 7.05-7.14 (m, J=8.6 Hz, 1H), 7.30-7.37 (m, 1H), 7.48 (d, J=8.1 Hz, 1H), 7.87 (s, 1H), 7.98 (br s, 1H), 8.44 (s, 1H), 9.47 (br s, 1H), 11.81 (br s, 1H).

Step 3″. Preparation of 4-(cyclopropylamino)-6-[(2-oxo-3H-1,3-benzothiazol-6-yl)amino]pyridine-3-carboxylic acid (A-59): In a round-bottom flask were added ethyl 4-(cyclopropylamino)-6-[(2-oxo-3H-1,3-benzothiazol-6-yl)amino]pyridine-3-carboxylate 5′ (100 mg, 0.27 mmol, 1 eq.), lithium hydroxide monohydrate (32 mg, 1.35 mmol, 5 eq.), THF (1 mL), MeOH (1 mL), and water (1 mL). The reaction was stirred at 60° C. After 6 h, LCMS showed complete conversion. Volatiles were removed under vacuum and the residue taken up with water (5 mL). A 6 M aqueous solution of HCl was added to reach pH 3. The precipitate was filtered off and dried under vacuum to give A-59 (73 mg, 0.213 mmol, 79% yield) as brown solid.

LCMS method 1: 85.2% purity at 215 nm, [M+H]⁺=343.1.

¹H NMR (400 MHz, DMSO-d6) δ ppm 0.56-0.65 (m, 2H), 0.76-0.85 (m, 2H), 2.54-2.59 (m, 1H), 6.34 (s, 1H), 7.18 (d, J=8.6 Hz, 1H), 7.26-7.36 (m, 1H), 7.76 (s, 1H), 8.32 (s, 1H), 8.41-8.58 (m, 1H), 9.95 (br s, 1H), 11.99 (s, 1H), 13.22 (br s, 1H).

TBM intermediates prepared using General Procedure TBM-1 are summarized in Table 14.

TABLE 14
TBM Intermediates Prepared via General Procedure TBM-1

| | Structure | Heterobicycle | Amine |
|---|---|---|---|
| A-1 | 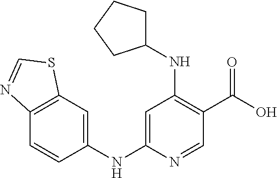 [SMILES: O=C(O)c1cnc(Nc2ccc3ncsc3c2)cc1NC1CCCC1] | 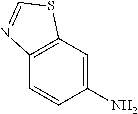 | 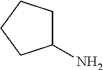 |
| A-2 | 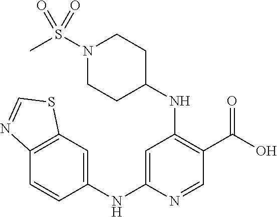 [SMILES: CS(=O)(=O)N1CCC(Nc2cc(Nc3ccc4ncsc4c3)ncc2C(=O)O)CC1] | 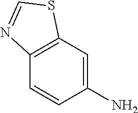 | 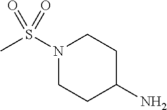 |
| A-5 | 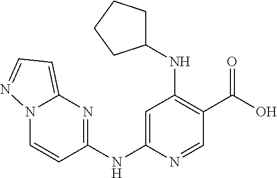 [SMILES: O=C(O)c1cnc(Nc2ccn3nccc3n2)cc1NC1CCCC1] | 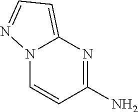 | 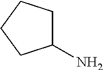 |
| A-6 | 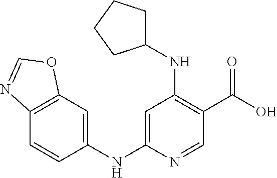 [SMILES: O=C(O)c1cnc(Nc2ccc3ncoc3c2)cc1NC1CCCC1] | 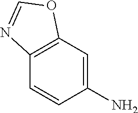 | 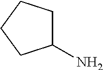 |
| A-53 | 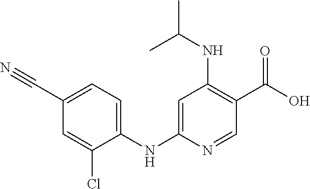 [SMILES: CC(C)Nc1cc(Nc2ccc(C#N)cc2Cl)ncc1C(=O)O] | 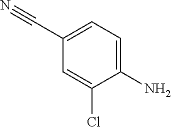 | 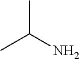 |
| A-56 | 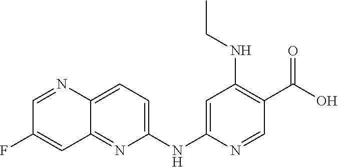 [SMILES: CCNc1cc(Nc2ccc3ncc(F)cc3n2)ncc1C(=O)O] | 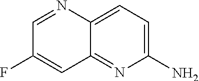 [SMILES: Nc1ccc2ncc(F)cc2n1] | 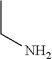 |
| A-59 | 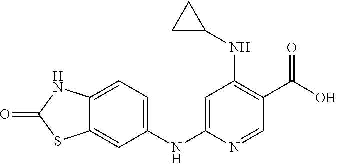 [SMILES: O=C(O)c1cnc(Nc2ccc3[nH]c(=O)sc3c2)cc1NC1CC1] | 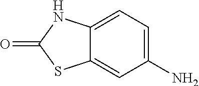 [SMILES: Nc1ccc2[nH]c(=O)sc2c1] | 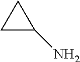 |

### General Procedure TBM-2

### Example S22. 1-(4-(cyclopentylamino)-5-ethynylpyridin-2-yl)-1H-pyrazolo[3,4-b]pyridine-5-carbonitrile (A-9)

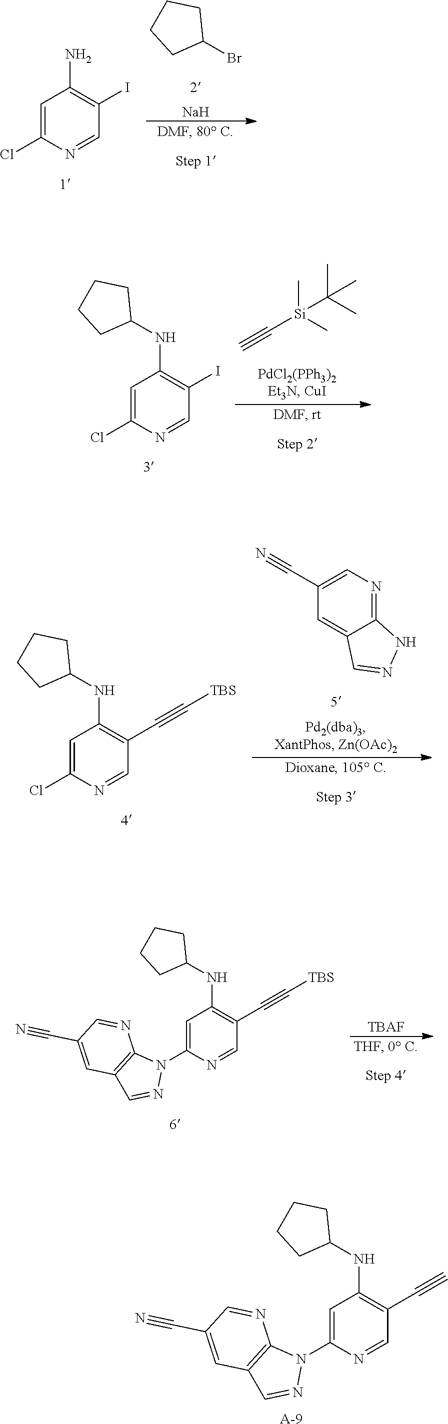

Step 1′. Preparation of 2-chloro-N-cyclopentyl-5-iodopyridin-4-amine (3′): To a stirred solution of 2-chloro-5-iodo-pyridin-4-amine (1.25 g, 4.91 mmol) (1′) in DMF (6.7293 mL) at 0° C. was added NaH (654.98 mg, 9.82 mmol) in small portions, the reaction was stirred at room temperature for 1 hour. The reaction mixture was heated to 80° C. and stirred for 30 minutes at this temperature. Bromocyclopentane (1.05 mL, 9.82 mmol) (2′) was added dropwise at that temperature and the reaction was stirred at 80° C. overnight. The reaction was quenched with NH₄Cl(sat) solution and extracted with EtOAc. The org phase was washed with brine (×3). The aq. phases were extracted with EtOAc (×3). Combined org phases were washed with brine and dried (MgSO₄) before concentration to dryness. Purification by normal phase flash chromatography, pre-adsorbed on SiO2 onto a 80 g SNAP column, eluted from 1 to 50% EtOAc/heptanes over 10 CVs. The desired product came out in the first UV active peak (18% EA), fractions were combined and concentrated under reduce pressure to afford 1.2 g (28% yield) of the desired product (3′) and 1.69 g of starting material was recovered.

LCMS (3.0 min run, Low pH): rt=1.9670 min; [M+H]⁺=323.0 m/z

Step 2′. Preparation of 5-((tert-butyldimethylsilyl)ethynyl)-2-chloro-N-cyclopentylpyridin-4-amine (4′): 2-chloro-N-cyclopentyl-5-iodo-pyridin-4-amine (2.6 g, 8.06 mmol) (3′), tert-butyl-ethynyl-dimethyl-silane (1.43 mL, 7.66 mmol), CuI (383.77 mg, 2.02 mmol), NEt₃ (22.47 mL, 161.21 mmol) in DMF (37.201 mL) were degassed with N₂. Then, PdCl₂(PPh₃)₂ (565.75 mg, 0.8100 mmol) was added to the mixture, it was bubbled for 10 minutes with N₂. The reaction was stirred at room temperature until full conversion was observed by LCMS. The crude mixture was diluted with H₂O and was extracted with MTBE (2×), MeTHF (2×) the organic layers were combined. The organic layers were washed with HCl 1M, NaHCO₃ (sat.) and brine, it was dried with MgSO₄, filtered and concentrated under reduce pressure for purification. Purification via normal phase chromatography, pre-adsorbed on SiO2, eluted onto a 80 g SNAP column from 2 to 20% EtOAc/heptane over 15 CVs, product eluted at 8% EA. The fractions from the major peak were combined and concentrated under reduce pressure to afford 2.75 g (66% yield) of the desired product (4′) as a white solid.

LCMS (3.5 min run, Low pH): pdt rt=2.499 min; [M+H]⁺=335.4 m/z.

Step 3′. Preparation of 1-(5-((tert-butyldimethylsilyl)ethynyl)-4-(cyclopentylamino)pyridin-2-yl)-1H-pyrazolo[3,4-b]pyridine-5-carbonitrile (6′): 5-[2-[tert-butyl(dimethyl)silyl]ethynyl]-2-chloro-N-cyclopentyl-pyridin-4-amine (2.75 g, 8.21 mmol) (4′), 1H-pyrazolo[3,4-b]pyridine-5-carbonitrile (0.66 mL, 9.85 mmol) (5′), Xantphos (1520.14 mg, 2.63 mmol), Zn(OAc)₂ (1506.36 mg, 8.21 mmol) in 1,4-Dioxane (50 mL) were degassed with N₂. Then, Pd₂(dba)₃·CHCl₃ (1127.7 mg, 1.23 mmol) was added to the mixture, it was bubbled for 10 minutes with N₂. The rxn mixture was stirred at 105° C. for 10 h and showed incomplete conversion (˜50%). At RT additional Zn(OAc)₂ (753.18 mg, 4.1 mmol), Xantphos (1425.13 mg, 2.46 mmol), Pd₂(dba)₃ (751.8 mg, 0.8200 mmol) were added to the mixture, and was bubbled for 10 minutes with N₂. The rxn mixture was stirred at 105° C. for another 10 h, where LCMS showed full conversion. The reaction mixture was passed over celite, washed with MeTHF, the solvent has been concentrated under reduce pressure prior to purification via normal phase flash chromatography, pre-adsorbed on SiO2, eluted onto a 80 g SNAP column from 1 to 65% EtOAc/heptanes over 15 CVs. The fractions from the last peak (ca. 58% EtOAc/heptanes) were combined and concentrated under reduce pressure to afford final product (6′) 1.5 g (41% yield).

LCMS (2.5 min run, Low pH): rt=2.278 min; [M+H]⁺=443.2 m/z.

Step 4′. Preparation of 1-(4-(cyclopentylamino)-5-ethynylpyridin-2-yl)-1H-pyrazolo[3,4-b]pyridine-5-carbonitrile (A-9): To a stirring solution of 1-[5-[2-[tert-butyl(dimethyl)silyl]ethynyl]-4-(cyclopentylamino)-2-pyridyl]pyrazolo[3,4-b]pyridine-5-carbonitrile (265. mg, 0.6000 mmol) in THF (5.9869 mL) was added, at 0° C., a solution of TBAF (2.39 mL, 2.39 mmol) 1 M in THF. The mixture was stirred while warming up to room temperature until LCMS demonstrated full conversion. The crude mixture was concentrated to dryness. Then, it was quenched with NH₄C₁ and diluted in EtOAc. The organic layer was washed with NaHCO₃ and brine, dried over MgSO₄, filtered and concentrated under reduce pressure before purification via reverse FC (MeOH/0.1% FA aq., 5% to 100% MeOH in 17 CV, product exit at 41% MeOH) to afford A-9, M=80 mg, 95% pure at 215 nm.

LCMS (2.5 min run, Low pH): 1.677 min; [M+H]⁺=329.1 m/z.

TBM intermediates prepared using General Procedure TBM-2 are summarized in Table 15.

TABLE 15
TBM Intermediates Prepared via General Procedure TBM-2

| | Structure | Heterobicycle | Amine |
|---|---|---|---|
| A-3 | 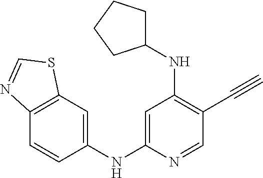 [SMILES: C#Cc1cnc(Nc2ccc3ncsc3c2)cc1NC1CCCC1] | 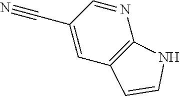 [SMILES: N#Cc1cnc2[nH]ccc2c1] | 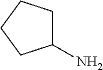 |
| A-9 | 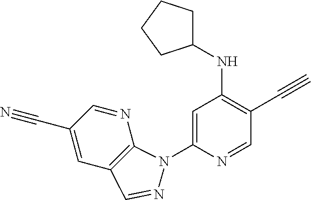 [SMILES: C#Cc1cnc(-n2ncc3cc(C#N)cnc32)cc1NC1CCCC1] | 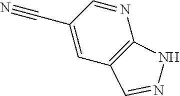 [SMILES: N#Cc1cnc2[nH]ncc2c1] | 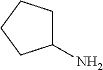 |

### General Procedure TBM-3

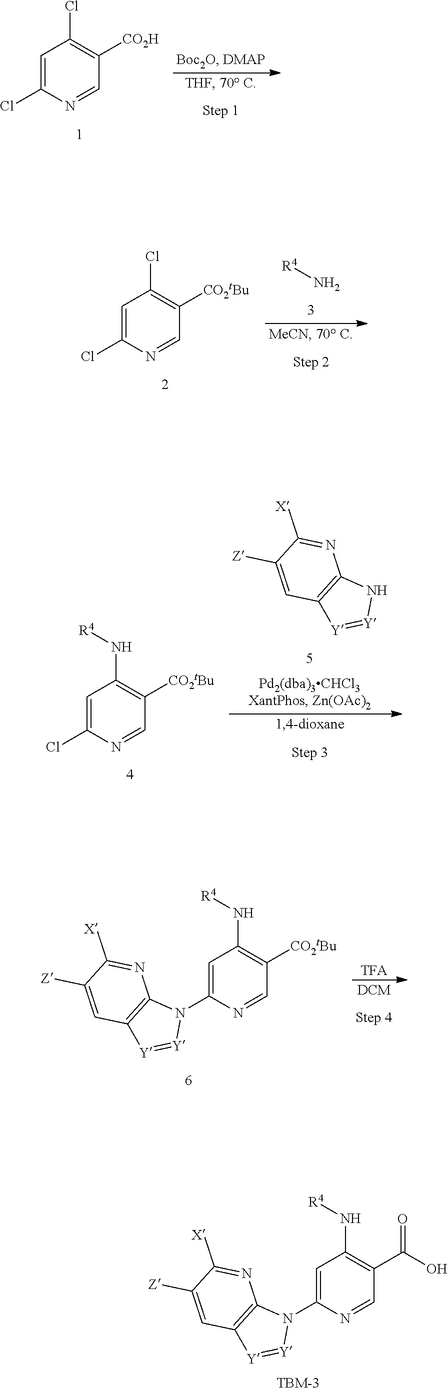

R⁴ is as defined in Formula (I′) or (I) and X′, Y′, and Z′ are selected from C, N, O, and S.

Step 1. Preparation of tert-butyl 4,6-dichloropyridine-3-carboxylate (2): To a solution of 4,6-dichloropyridine-3-carboxylic acid 1 (1 eq.) and DMAP (0.2 eq.) in THF (0.52 M) at 70° C. was added dropwise tert-butoxycarbonyl tert-butyl carbonate (2 eq.) in THF (0.52 M), over 2 hours with an addition funnel. The reaction mixture was then stirred at 70° C. for another hour. THF was evaporated under reduced pressure and the residue was purified by normal phase flash chromatography to affrod product 2.

Step 2. Preparation of tert-butyl 6-chloro-4-(amino)pyridine-3-carboxylate (4): To a solution of tert-butyl 4,6-dichloropyridine-3-carboxylate 2 (1 eq.) in MeCN (0.45 M) was added amine 3 (3 eq.). The reaction was stirred at 70° C. Upon LCMS indication of conversion, the reaction mixture was cooled to room temperature, water (350 mL) was added, a white semi-solid formed. The resulting slurry was stirred for 1 h at 0° C. The aqueous solution was extracted 2×DCM and 2×MeTHF. The solvent was evaporated under reduced pressure and the residue was purified by normal phase flash chromatography to afford product 4.

In some cases, DIPEA was added to help promote the SNAr reaction, particularly when using a salt form of an amine.

Step 3. Preparation of tert-Butyl 6-(heterobicyclic)-4-(amino)pyridine-3-carboxylate (6): A round bottomed flask was loaded with 4 (1.0 eq.), 5 (1.1 eq.), XantPhos (0.3 eq.), Zn(OAc)₂ (0.6 eq.) and Pd₂(dba)₃·CHCl₃ (0.15 eq.). 1,4-dioxane (0.2 M) was added, the mixture was sonicated and then nitrogen was sparged through the mixture for 5 min. The mixture was stirred at 110° C. for 16 h. The mixture was passed over Celite, washed with DCM. The solution was dry-packed with silica. Purification by normal phase flash chromatography (heptanes/EtOAc) afforded 6.

Step 4. Preparation TBM-3: TFA (30 eq.) was added to a solution of 6 (1.0 eq.) in DCM (0.9 M) at room temperature. The solution was stirred at 45° C. for 2 h. Solvent was removed under vacuum. Residual TFA was co-evaporated with toluene (2×) then with MeCN. The residue was dried under high vacuum to give a yellow solid. MTBE was added to the solid and the mixture was sonicated and stirred at room temperature for 16 h. The suspension was filtrated over a Buchner funnel and rinced with MTBE to give TBM-3.

Similar acidic conditions can be used in the ester hydroylsis.

### Example S23. 6-(5-cyanopyrazolo[3,4-b]pyridin-1-yl)-4-(cyclopentylamino)pyridine-3-carboxylic acid (A-7)

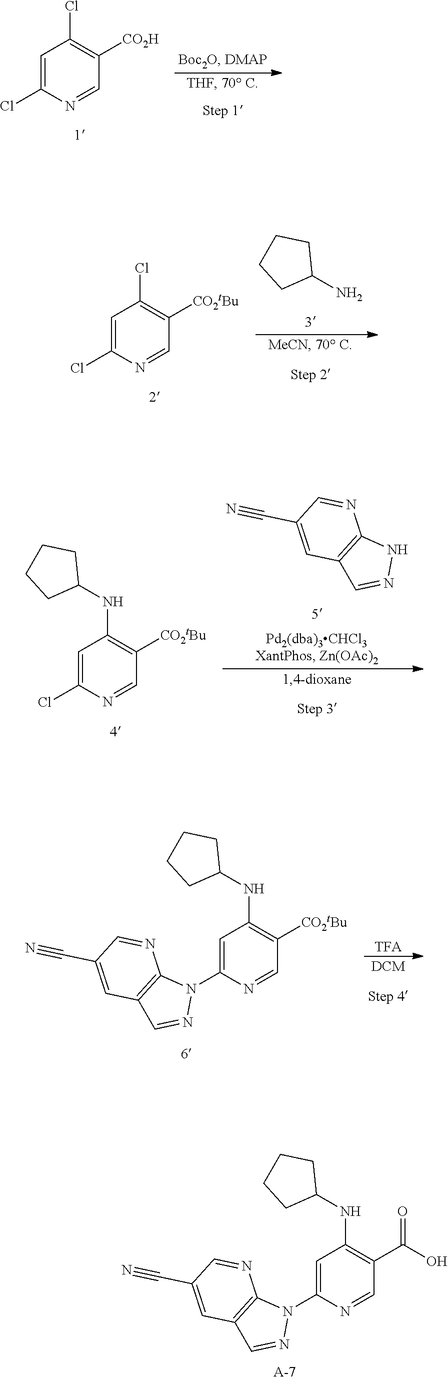

LCMS Method 1. Column: Luna C18(2) 50×3 mm, 3 um. Temperature: 45 C, Flow: 1.5 mL/min, Run time: 2.5 min. Mobile phase conditions: Initial 95% H₂O 0.1% FA/5% MeCN 0.1% FA, linear gradient to 95% MeCN 0.1% FA over 1.3 min then hold for 1.2 minute at 95% MeCN 0.1% FA. MSD: ESI Positive

Step 1′. Preparation of tert-butyl 4,6-dichloropyridine-3-carboxylate (2′): To a solution of 4,6-dichloropyridine-3-carboxylic acid 1′ (20.0 g, 104.2 mmol, 1 eq.) and DMAP (2.5 g, 20.8 mmol, 0.2 eq.) in THF (150 mL, 0.52 M) at 70° C. was added dropwise tert-butoxycarbonyl tert-butyl carbonate (45.4 g, 208.3 mmol, 2 eq.) in THF (50 mL, 0.52 M), over 2 hours with an addition funnel. The reaction mixture was then stirred at 70° C. for another hour. THF was evaporated under reduced pressure and the residue was purified by normal phase flash chromatography (380 g column, solid deposit, elution 0 to 15% Hept./EtOAc over 15 CV). Fractions were combined and concentrated, affording 2′ (22 g, 85% yield) as a light yellow oil.

LCMS method 1: 99.9% purity at 215 nm [M+H]⁺=248.2.

¹H NMR (400 MHz, chloroform-d) δ ppm 1.63 (s, 9H), 7.45 (s, 1H), 8.77 (s, 1H).

Step 2′. Preparation of tert-butyl 6-chloro-4-(cyclopentylamino)pyridine-3-carboxylate (4′): To a solution of tert-butyl 4,6-dichloropyridine-3-carboxylate 2′ (2.02 g, 8.13 mmol, 1 eq.) in MeCN (18 mL, 0.45 M) was added cyclopentanamine 3′ (2.41 mL, 24.38 mmol, 3 eq.). The reaction was stirred at 70° C. After 2.5 h, LCMS showed complete conversion into compound 4′. The reaction mixture was cooled to room temperature, water (350 mL) was added, a white semi-solid formed. The resulting slurry was stirred for 1 h at 0° C. No precipitate formed. The aqueous solution was extracted 2×DCM and 2×MeTHF. The solvent was evaporated under reduced pressure and the residue was purified by normal phase flash chromatography (40 g column, solid deposit, elution 0% to 30% EtOAc/Heptane over 15 CV). Fractions were combined and concentrated, affording 4′ (2.4 g, 99% yield) as a colorless oil.

LCMS method 1: 99.9% purity at 215 nm [M+H]⁺=297.2.

¹H NMR (400 MHz, chloroform-d) δ ppm 1.45-1.54 (m, 11H), 1.55-1.64 (m, 2H), 1.66-1.76 (m, 2H), 1.94-2.05 (m, 2H), 3.67-3.78 (m, 1H), 6.46 (s, 1H), 8.12 (br. d, J=Hz, 1H), 8.50 (s, 1H).

Step 3′. Preparation of tert-butyl 6-(5-cyanopyrazolo[3,4-b]pyridin-1-yl)-4-(cyclopentylamino)pyridine-3-carboxylate (6′): To a flame dried sealed tube was added tert-butyl 6-chloro-4-(cyclopentylamino)pyridine-3-carboxylate 4′ (1.0 g, 3.37 mmol, 1 eq.), 1H-pyrazolo[3,4-b]pyridine-5-carbonitrile 5′ (631.3 mg, 4.38 mmol, 1.3 eq.), Xantphos (623.8 mg, 1.08 mmol, 0.3 eq.), Zn(OAc)₂ (197.8 mg, 1.08 mmol, 0.3 eq.) and Pd₂(dba)₃·CHCl₃ (523.1 mg, mmol, 0.15 eq.) in 1,4-dioxane (13.5 mL, 0.25 M). The solution was sparged with nitrogen for 35 min, sealed and stirred at 110° C. After an overnight period, LCMS showed complete conversion into compound 6′. The mixture was passed over celite and the pad was washed with DCM. The solvent was evaporated under reduced pressure and the residue was purified by normal phase flash chromatography (40 g gold column, solid deposit, elution 20 to 100% EtOAc/Heptane over 15 CV). Fractions were combined and concentrated, affording 6′ (1.29 g, 87% yield) as a yellow solid.

LCMS method 1: 92.3% purity at 215 nm [M+H]⁺=405.2.

¹H NMR (400 MHz, chloroform-d) δ ppm 1.55 (s, 9H), 1.60-1.72 (m, 4H), 1.75-1.84 (m, 2H), 2.10-2.18 (m, 2H), 3.92-4.03 (m, 1H), 7.82 (br. s, 1H), 8.56 (s, 1H), 8.68 (d, J=1.7 Hz, 1H), 8.74 (s, 1H), 8.93 (br. s, 1H), 8.98 (d, J=2.0 Hz, 1H).

Step 4′. Preparation of 6-(5-cyanopyrazolo[3,4-b]pyridin-1-yl)-4-(cyclopentylamino)pyridine-3-carboxylic acid (A-7): To a solution of tert-butyl 6-(5-cyanopyrazolo[3,4-b]pyridin-1-yl)-4-(cyclopentylamino)pyridine-3-carboxylate 6′ (0.8 g, 1.98 mmol, 1 eq.) in anhydrous DCM (11.6 mL, 0.17 M), under nitrogen atmosphere was added TFA (4.8 mL, 59.3 mmol, 30 eq.) and the reaction was stirred at room temperature. After an overnight stirring, LCMS showed complete conversion into compound 7′. The solvent was removed under reduced pressure and the residue was co-evaporated with toluene (3×) followed by MeCN (2×), affording a yellow semi-solid to which was added H₂O (50 mL)/MeCN (10 mL) and the mixture was sonicated 3-5 min. The solids were stirred for 10 min at room temperature, filtered, and washed with water and MeCN (until white solid), affording A-7 (0.9 g, 99% yield) as a white solid.

LCMS method 1: 93.2% purity at 215 nm [M+H]⁺=349.2.

¹H NMR (400 MHz, chloroform-d) δ ppm 1.50-1.61 (m, 2H), 1.62-1.76 (m, 4H), 2.04-2.17 (m, 2H), 3.95-4.04 (m, 1H), 7.46 (s, 1H), 8.40 (br. d, J=6.4 Hz, 1H), 8.67 (s, 1 H), 8.77 (s, 1H), 9.03 (d, J=2.0 Hz, 1H), 9.09 (d, J=2.0 Hz, 1H), 13.25 (br. s, 1H).

### Example S24. 6-(6-amino-5-cyano-pyrazolo[3,4-b]pyridin-1-yl)-4-[(1-methylsulfonyl-4-piperidyl)amino]pyridine-3-carboxylic acid (A52)

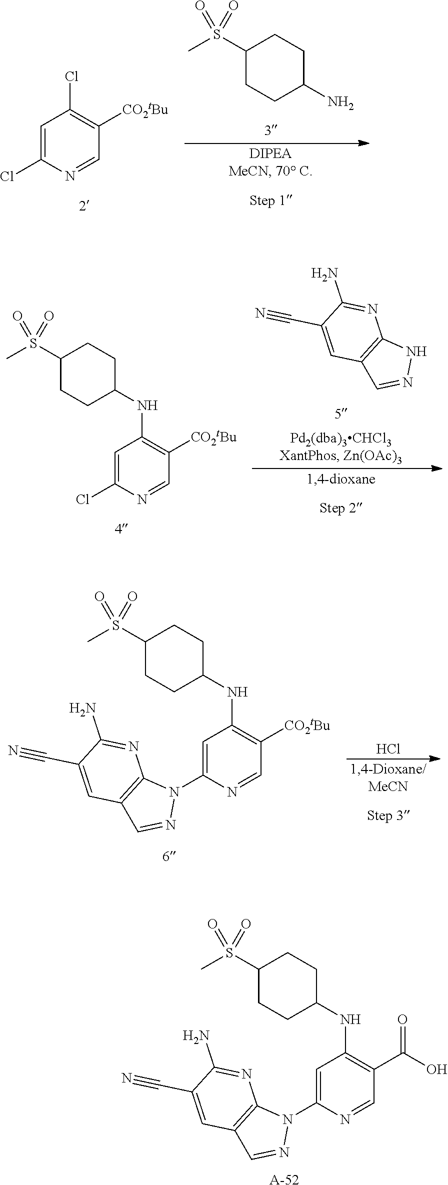

LCMS Method 1. Column: Luna C18 (2) 50×3 mm, 3 um. Temperature: 45° C., Flow: 1.5 mL/min, run time: 2.5 min. Mobile phase conditions: Initial 95% H₂O 0.1% FA/5% MeCN+0.1% FA, linear gradient to 95% MeCN 0.1% FA over 1.3 min then hold for 1.2 minute at 95 MeCN 0.1% FA. MSD: ESI Positive.

Step 1″. Preparation of tert-butyl 6-chloro-4-[(1-methylsulfonyl-4-piperidyl)amino]pyridine-3-carboxylate (4″): To a solution of 3″ (3.46 g, 19.41 mmol) and 2′ (1.26 g, 5.08 mmol) in MeCN (19 mL) was added DIPEA (5.3 mL, 30.47 mmol) at RT and the resulting mixture was stirred at 65° C. After 4 days, volatiles were evaporated and the residue was partitioned between EtOAc and water. The aqueous phase was extracted to EtOAc (2×). Combined organic phases were washed with saturated NaHCO₃ and brine, dried over MgSO₄ and concentrated under reduced pressure. The residue was purified by silica gel flash column chromatography (EtOAc/heptanes) to afford 4″ (1.9 g, 94% yield) as a white solid.

LCMS method 1: 98.1% purity at 215 nm, [M+H]⁺=390.1.

¹H NMR (400 MHz, CDCl₃) δ ppm 1.58 (s, 9H), 1.75 (dtd, J=13.4, 9.7, 3.8 Hz, 2H), 2.10-2.19 (m, 2H), 2.84 (s, 3H), 2.99-3.09 (m, 2H), 3.48-3.58 (m, 1H), 3.72 (dt, J=12.3, 4.1 Hz, 2H), 6.51 (s, 1H), 8.38 (d, J=7.6 Hz, 1H), 8.63 (s, 1H).

Step 2″. Preparation of tert-butyl 6-(6-amino-5-cyano-pyrazolo[3,4-b]pyridin-1-yl)-4-[(1-methylsulfonyl-4-piperidyl)amino]pyridine-3-carboxylate (6″): A sealed tube was charged with 4″ (300 mg, 0.77 mmol), 5″ (134 mg, 0.85 mmol), Xantphos (133 mg, 0.23 mmol), Pd₂(dba)₃·CHCl₃ (119 mg, 0.12 mmol), Zn(OAc)₂ (141 mg, 0.77 mmol) in 1,4-dioxane (3.8 mL). Then nitrogen was sparged through the mixture for 15 minutes. The tube was sealed and reaction mixture was heated at 105° C. for 16 h. The reaction mixture was cooled down to room temperature and filtered through a pad of celite. The filter cake was washed with DCM and the filtrate was concentrated to dryness. The residue was purified by reverse-phase C18 flash chromatography (MeOH/0.1% aqueous formic acid) to afford tert-butyl 6″ (127 mg, 32% yield) as a yellow solid.

LCMS method 1: 99.9% purity at 215 nm, [M+H]⁺=513.2.

¹H NMR (400 MHz, DMSO-d6) δ ppm 1.58 (s, 9H), 1.59-1.67 (m, 2H), 2.08-2.19 (m, 2H), 2.90 (s, 3H), 2.98-3.11 (m, 2H), 3.50-3.58 (m, 2H), 3.70-3.85 (m, 1H), 7.25 (s, 2H), 7.46 (s, 1H), 8.07 (br d, J=7.3 Hz, 1H), 8.20 (s, 1H), 8.54 (s, 1H), 8.71 (s, 1H).

Step 3″. Preparation of 6-(6-amino-5-cyano-pyrazolo[3,4-b]pyridin-1-yl)-4-[(1-methylsulfonyl-4-piperidyl)amino]pyridine-3-carboxylic acid (A-52): To a solution of 6″ (127 mg, 0.25 mmol) in MeCN (1.0 mL) was added 4 M HCl in 1,4-dioxane (3.1 mL, 12.39 mmol). The reaction mixture was stirred at room temperature. After 18 h, volatiles were removed in vacuo and the residue was co-evaporated with MeCN (2×). The crude solid was triturated in MeCN which afforded A-52 (115 mg, quant.) as a yellow solid.

LCMS method 1: 99.9% purity at 215 nm, [M+H]⁺=457.1.

¹H NMR (400 MHz, DMSO-d6) δ ppm 1.57-1.70 (m, 2H), 2.09-2.20 (m, 2H), 2.91 (s, 3H), 3.04-3.14 (m, 2H), 3.82-3.93 (m, 2H), 4.14 (dd, J=5.6, 3.4 Hz, 1H), 7.44 (d, J=3.9 Hz, 1H), 7.62-7.75 (m, 3H), 8.31 (s, 1H), 8.59 (s, 1H), 8.67 (s, 1H).

TBM intermediates prepared using General Procedure TBM-3 are summarized in Table 16.

TABLE 16
TBM Intermediates Prepared via General Procedure TBM-3

| | Structure | Heterobicycle | Amine |
|---|---|---|---|
| A-4 | 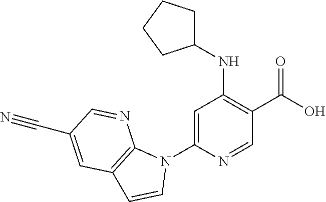 [SMILES: N#Cc1cnc2c(ccn2-c2cc(NC3CCCC3)c(C(=O)O)cn2)c1] | 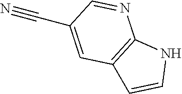 [SMILES: N#Cc1cnc2[nH]ccc2c1] | 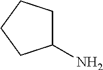 |
| A-8 | 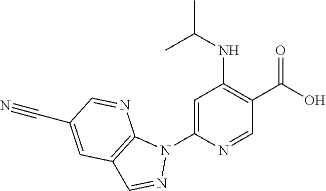 [SMILES: CC(C)Nc1cc(-n2ncc3cc(C#N)cnc32)ncc1C(=O)O] | 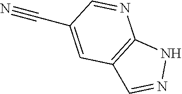 [SMILES: N#Cc1cnc2[nH]ncc2c1] | 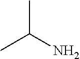 |
| A-11 | 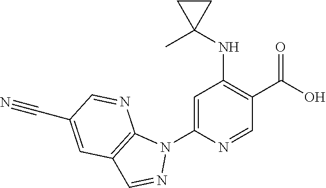 [SMILES: CC1(Nc2cc(-n3ncc4cc(C#N)cnc43)ncc2C(=O)O)CC1] | 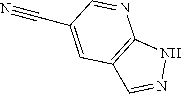 [SMILES: N#Cc1cnc2[nH]ncc2c1] | 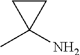 |
| A-14 | 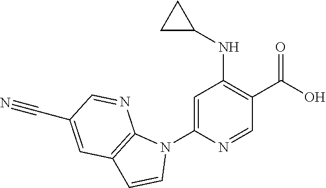 [SMILES: N#Cc1cnc2c(ccn2-c2cc(NC3CC3)c(C(=O)O)cn2)c1] | 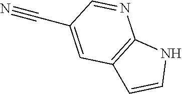 [SMILES: N#Cc1cnc2[nH]ccc2c1] | 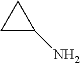 |
| A-16 | 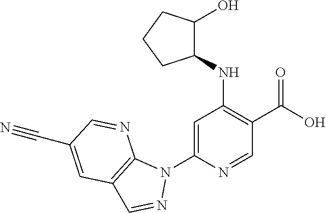 [SMILES: N#Cc1cnc2c(cnn2-c2cc(N[C@H]3CCCC3O)c(C(=O)O)cn2)c1] | 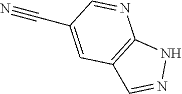 [SMILES: N#Cc1cnc2[nH]ncc2c1] | 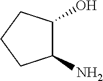 |
| A-18 | 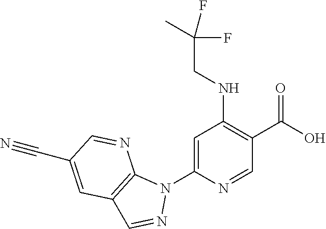 [SMILES: CC(F)(F)CNc1cc(-n2ncc3cc(C#N)cnc32)ncc1C(=O)O] | 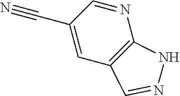 [SMILES: N#Cc1cnc2[nH]ncc2c1] | 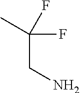 |
| A-20 | 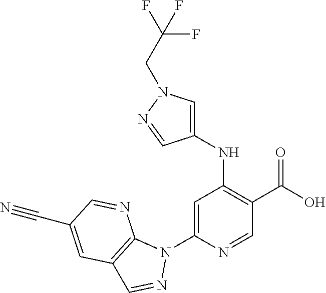 [SMILES: N#Cc1cnc2c(cnn2-c2cc(Nc3cnn(CC(F)(F)F)c3)c(C(=O)O)cn2)c1] | 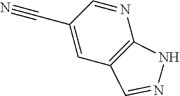 [SMILES: N#Cc1cnc2[nH]ncc2c1] | 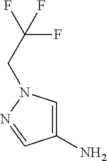 [SMILES: Nc1cnn(CC(F)(F)F)c1] |
| A-22 | 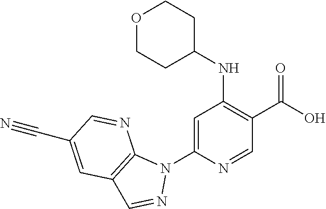 [SMILES: N#Cc1cnc2c(cnn2-c2cc(NC3CCOCC3)c(C(=O)O)cn2)c1] | 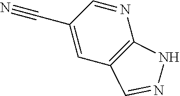 [SMILES: N#Cc1cnc2[nH]ncc2c1] | 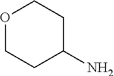 |
| A-25 | 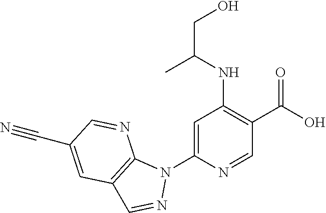 [SMILES: CC(CO)Nc1cc(-n2ncc3cc(C#N)cnc32)ncc1C(=O)O] | 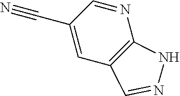 [SMILES: N#Cc1cnc2[nH]ncc2c1] | 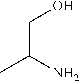 |
| A-27 | 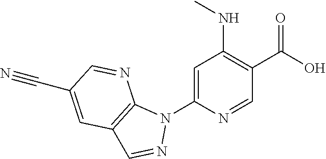 [SMILES: CNc1cc(-n2ncc3cc(C#N)cnc32)ncc1C(=O)O] | 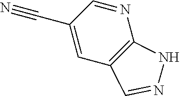 [SMILES: N#Cc1cnc2[nH]ncc2c1] | 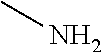 |
| | *Alternative Step 2 conditions: | | |
| | The tert-butyl 4,6-dichloropyridine-3-carboxylate (1.0 eq) | | |
| | was dissolved in MeCN (0.2 M) then a 2.0 M solution of | | |
| | methanamine (3.0 eq) in THF was added, followed by | | |
| | DIPEA (4.0 eq); the resulting solution was heated at the | | |
| | microwave at 80° C. for 30 minutes prior to addition of | | |
| | water and extraction with ethyl acetate. The organic | | |
| | fraction were combined, washed, dried and evaporated to | | |
| | form product. | | |
| A-30 | 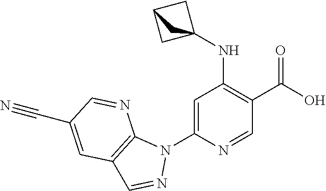 | 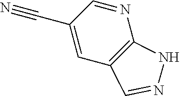 [SMILES: N#Cc1cnc2[nH]ncc2c1] | 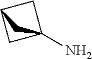 |
| A-33 | 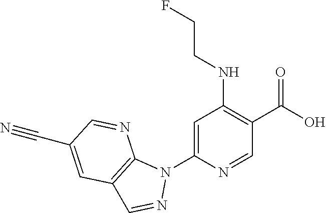 [SMILES: N#Cc1cnc2c(cnn2-c2cc(NCCF)c(C(=O)O)cn2)c1] | 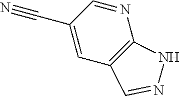 [SMILES: N#Cc1cnc2[nH]ncc2c1] | 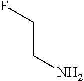 |
| A-35 | 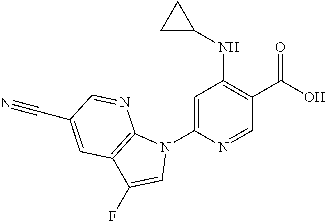 [SMILES: N#Cc1cnc2c(c1)c(F)cn2-c1cc(NC2CC2)c(C(=O)O)cn1] | 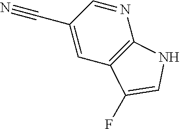 [SMILES: N#Cc1cnc2[nH]cc(F)c2c1] | 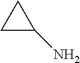 |
| A-37 | 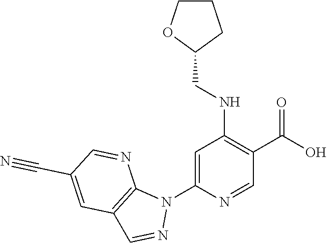 [SMILES: N#Cc1cnc2c(cnn2-c2cc(NC[C@H]3CCCO3)c(C(=O)O)cn2)c1] | 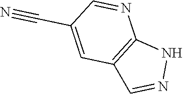 [SMILES: N#Cc1cnc2[nH]ncc2c1] | 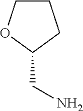 |
| A-39 | 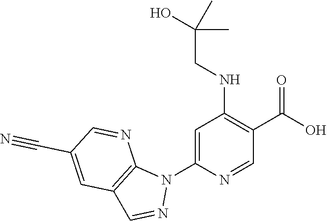 [SMILES: CC(C)(O)CNc1cc(-n2ncc3cc(C#N)cnc32)ncc1C(=O)O] | 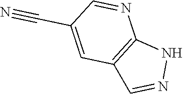 [SMILES: N#Cc1cnc2[nH]ncc2c1] | 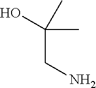 |
| A-4 [symbol] | 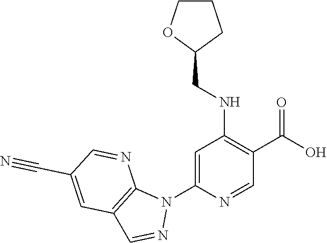 [SMILES: N#Cc1cnc2c(cnn2-c2cc(NC[C@@H]3CCCO3)c(C(=O)O)cn2)c1] | 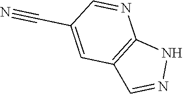 [SMILES: N#Cc1cnc2[nH]ncc2c1] | 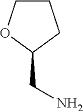 |
| A-45 | 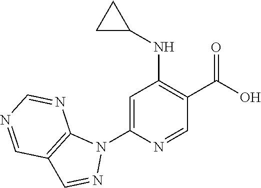 [SMILES: O=C(O)c1cnc(-n2ncc3cncnc32)cc1NC1CC1] | 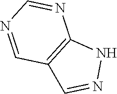 | 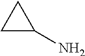 |
| A-47 | 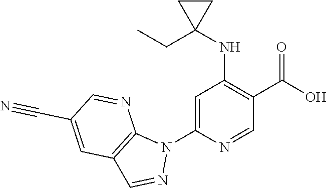 [SMILES: CCC1(Nc2cc(-n3ncc4cc(C#N)cnc43)ncc2C(=O)O)CC1] | 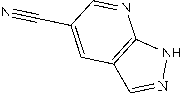 [SMILES: N#Cc1cnc2[nH]ncc2c1] | 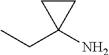 |
| A-51 | 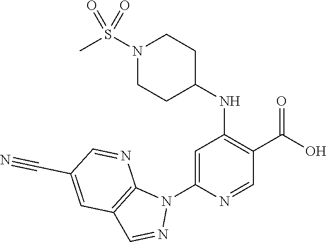 [SMILES: CS(=O)(=O)N1CCC(Nc2cc(-n3ncc4cc(C#N)cnc43)ncc2C(=O)O)CC1] | 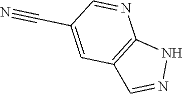 [SMILES: N#Cc1cnc2[nH]ncc2c1] | 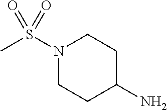 |
| A-7 | 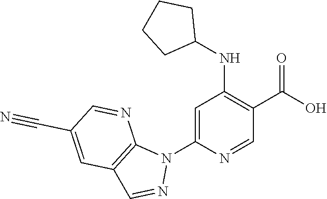 [SMILES: N#Cc1cnc2c(cnn2-c2cc(NC3CCCC3)c(C(=O)O)cn2)c1] | 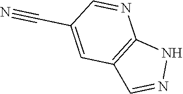 [SMILES: N#Cc1cnc2[nH]ncc2c1] | 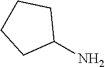 |
| A-10 | 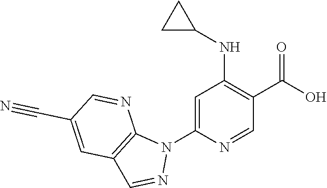 [SMILES: N#Cc1cnc2c(cnn2-c2cc(NC3CC3)c(C(=O)O)cn2)c1] | 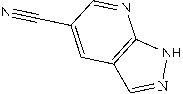 [SMILES: N#Cc1cnc2[nH]ncc2c1] | 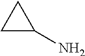 |
| A-12 | 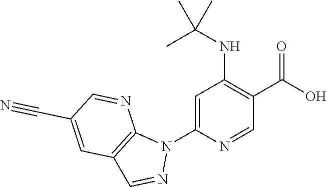 [SMILES: CC(C)(C)Nc1cc(-n2ncc3cc(C#N)cnc32)ncc1C(=O)O] | 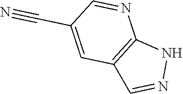 [SMILES: N#Cc1cnc2[nH]ncc2c1] | 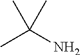 |
| A-15 | 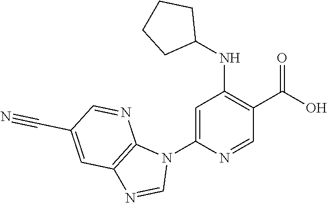 [SMILES: N#Cc1cnc2c(c1)ncn2-c1cc(NC2CCCC2)c(C(=O)O)cn1] | 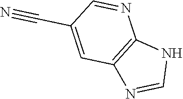 [SMILES: N#Cc1cnc2[nH]cnc2c1] | 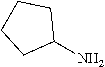 |
| | *C—N Coupling afforded a mixture of isomers that | | |
| | required separation | | |
| A-17 | 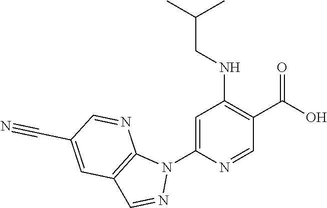 [SMILES: CC(C)CNc1cc(-n2ncc3cc(C#N)cnc32)ncc1C(=O)O] | 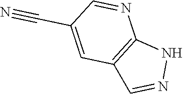 [SMILES: N#Cc1cnc2[nH]ncc2c1] | 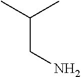 |
| A-19 | 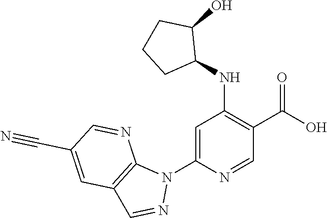 [SMILES: N#Cc1cnc2c(cnn2-c2cc(N[C@H]3CCC[C@H]3O)c(C(=O)O)cn2)c1] | 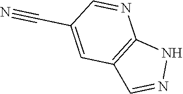 [SMILES: N#Cc1cnc2[nH]ncc2c1] | 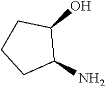 |
| A-21 | 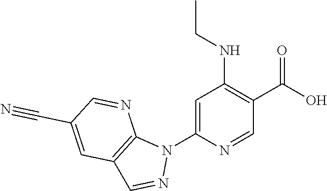 [SMILES: CCNc1cc(-n2ncc3cc(C#N)cnc32)ncc1C(=O)O] | 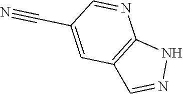 [SMILES: N#Cc1cnc2[nH]ncc2c1] | 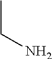 |
| A-24 | 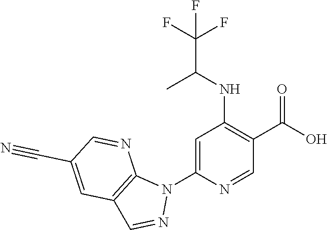 [SMILES: CC(Nc1cc(-n2ncc3cc(C#N)cnc32)ncc1C(=O)O)C(F)(F)F] | 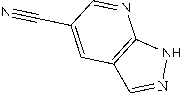 [SMILES: N#Cc1cnc2[nH]ncc2c1] | 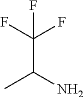 |
| A-26 | 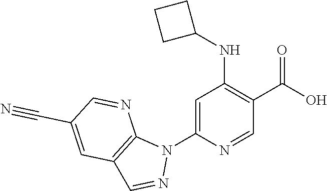 [SMILES: N#Cc1cnc2c(cnn2-c2cc(NC3CCC3)c(C(=O)O)cn2)c1] | 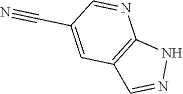 [SMILES: N#Cc1cnc2[nH]ncc2c1] | 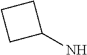 |
| A-28 | 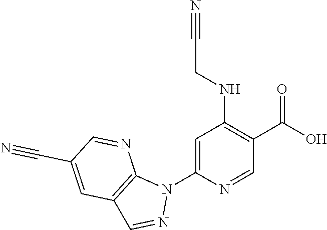 [SMILES: N#CCNc1cc(-n2ncc3cc(C#N)cnc32)ncc1C(=O)O] | 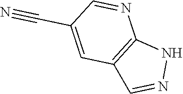 [SMILES: N#Cc1cnc2[nH]ncc2c1] | 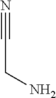 |
| A-32 | 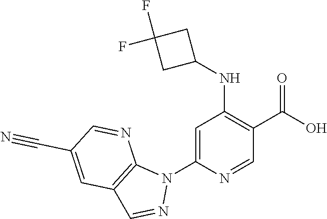 [SMILES: N#Cc1cnc2c(cnn2-c2cc(NC3CC(F)(F)C3)c(C(=O)O)cn2)c1] | 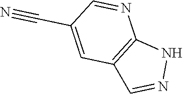 [SMILES: N#Cc1cnc2[nH]ncc2c1] | 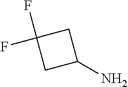 |
| A-34 | 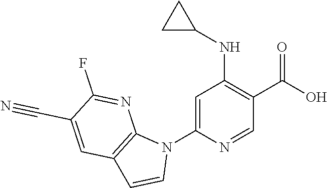 [SMILES: N#Cc1cc2ccn(-c3cc(NC4CC4)c(C(=O)O)cn3)c2nc1F] | 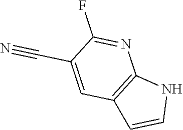 [SMILES: N#Cc1cc2cc[nH]c2nc1F] | 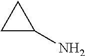 |
| A-36 | 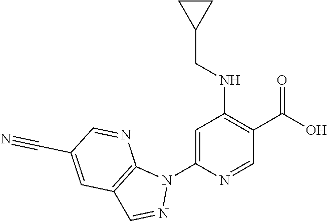 [SMILES: N#Cc1cnc2c(cnn2-c2cc(NCC3CC3)c(C(=O)O)cn2)c1] | 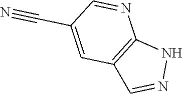 [SMILES: N#Cc1cnc2[nH]ncc2c1] | 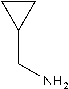 |
| A-38 | 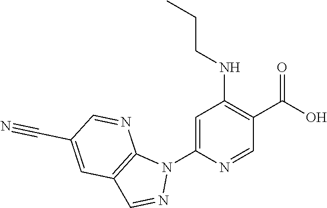 [SMILES: CCCNc1cc(-n2ncc3cc(C#N)cnc32)ncc1C(=O)O] | 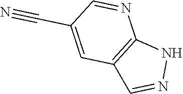 [SMILES: N#Cc1cnc2[nH]ncc2c1] | 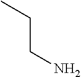 |
| A-41 | 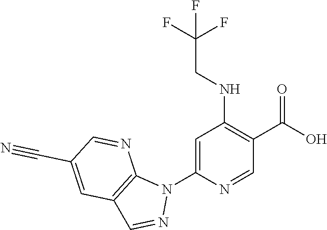 [SMILES: N#Cc1cnc2c(cnn2-c2cc(NCC(F)(F)F)c(C(=O)O)cn2)c1] | 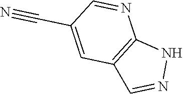 [SMILES: N#Cc1cnc2[nH]ncc2c1] | 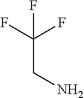 |
| A-43 | 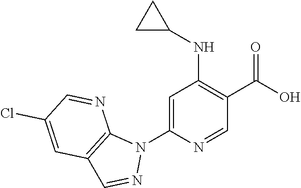 [SMILES: O=C(O)c1cnc(-n2ncc3cc(Cl)cnc32)cc1NC1CC1] | 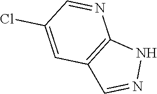 | 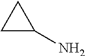 |
| A-46 | 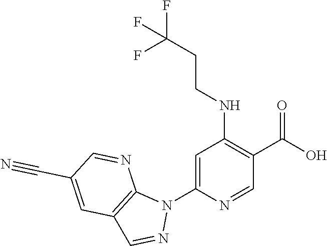 [SMILES: N#Cc1cnc2c(cnn2-c2cc(NCCC(F)(F)F)c(C(=O)O)cn2)c1] | 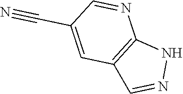 [SMILES: N#Cc1cnc2[nH]ncc2c1] | 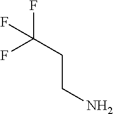 |
| A-50 | 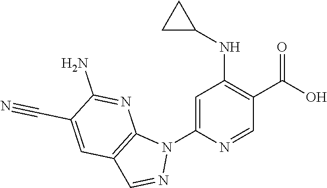 [SMILES: N#Cc1cc2cnn(-c3cc(NC4CC4)c(C(=O)O)cn3)c2nc1N] | 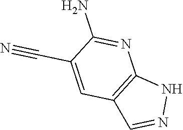 [SMILES: N#Cc1cc2cn[nH]c2nc1N] | 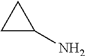 |
| A-52 | 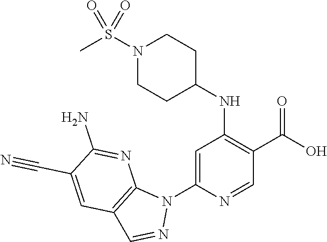 [SMILES: CS(=O)(=O)N1CCC(Nc2cc(-n3ncc4cc(C#N)c(N)nc43)ncc2C(=O)O)CC1] | 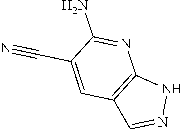 [SMILES: N#Cc1cc2cn[nH]c2nc1N] | 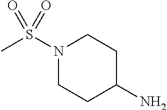 |

[symbol]

indicates data missing or illegible when filed

### General Procedure TBM-4

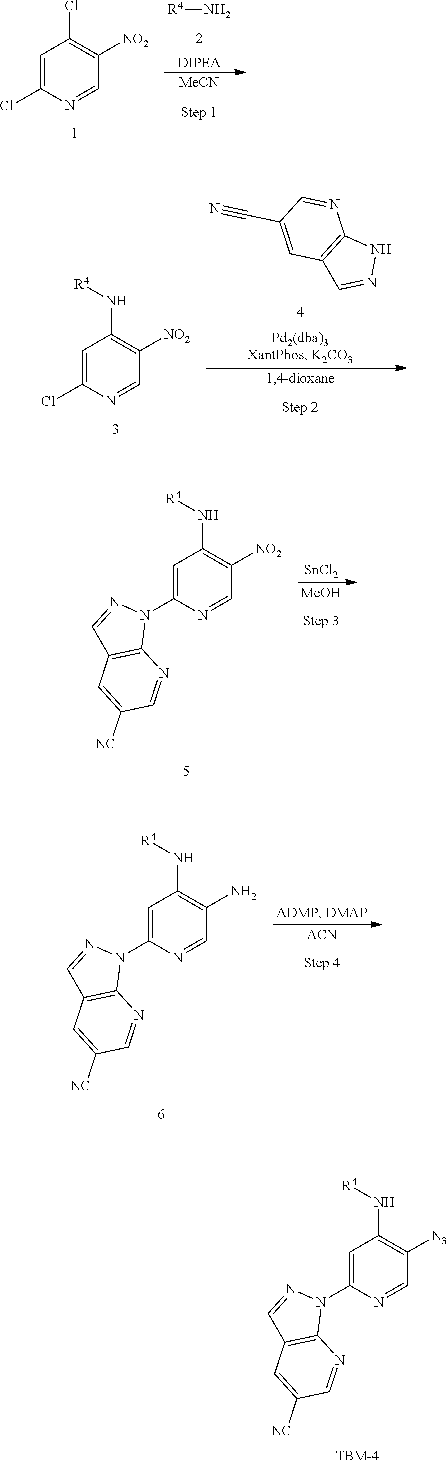

wherein R⁴ is as defined in Formula (I′) or (I).

Step 1. Preparation of 2-chloro-N-alkyl-5-nitropyridin-4-amine 3: To a solution of 2,4-dichloro-5-nitropyridine 1 (1.0 equiv.) in acetonitrile (1.0 M) was added alkyl amine 2 (1.5 equiv.) and DIPEA (2 equiv.) at RT and this resulting solution was stirred at 40° C. for 2 h. The solution was then cooled to room temperature and concentrated under reduced pressure (70% volume). Finally, ice-cold water was added to the reaction mixture and stirred for 10 min to afford a pale yellow solid. The solid was filtered, washed with water, and dried to give compound 3.

Step 2. Preparation of 1-(4-(alkyl-amino)-5-nitropyridin-2-yl)-1H-pyrazolo[3,4-b]pyridine-5-carbonitrile 5: To a solution of 2-chloro-N-alkyl-5-nitropyridin-4-amine 3 (1.0 equiv) in 1,4-dioxane was added 1H-pyrazolo[3,4-b]pyridine-5-carbonitrile 4 (1.2 equiv.), Xantphos (0.1 equiv) and K₂CO₃ (2.0 equiv). The resulting mixture was purged with nitrogen gas for 10 min, then was added Pd₂dba₃ (0.05 equiv.). The resulting mixture was stirred at 100° C. for 16 h. The reaction mixture was cooled to RT and filtered through celite bed, washed with ethyl acetate, and concentrated under reduced pressure to give the crude product. The crude was purified by flash column chromatography on silica gel to give pure compound 5.

Step 3. Preparation of 1-(5-amino-4-(alkyl-amino)pyridin-2-yl)-1H-pyrazolo[3,4-b]pyridine-5-carbonitrile 6: To a stirred solution of 1-(4-(alkyl-amino)-5-nitropyridin-2-yl)-1H-pyrazolo[3,4-b]pyridine-5-carbonitrile 5 (1.5 equiv.) in MeOH (0.15 M) at RT was added tin(II) chloride (5.0 equiv). The resulting solution was stirred at 70° C. for 16 h. The reaction mixture was cooled to RT and treated with ice-cold sat. sodium bicarbonate (50 mL) solution and extracted with 10% methanol in DCM (3×50 mL). The combined organic layer was dried over sodium sulphate and concentrated under reduced pressure to give the crude compound. The crude product was purified by flash column chromatography using silica gel with methanol/DCM to afford compound 6.

Step 4. Preparation of 1-(5-azido-4-(alkyl-amino)pyridin-2-yl)-1H-pyrazolo[3,4-b]pyridine-5-carbonitrile TBM-4: To a solution of 1-(5-amino-4-(alkyl-amino)pyridin-2-yl)-1H-pyrazolo[3,4-b]pyridine-5-carbonitrile 6 (1.0 equiv.) in acetonitrile (0.3 M) was added DMAP (1.5 equiv.) and ADMP (1.5 equiv.). The resulting solution was stirred for 16 h at RT. The reaction mixture was diluted with ice-cold water and extracted with ethyl acetate (3×20 mL). The combined organic layer was washed with brine, dried over sodium sulphate and then concentrated under reduced pressure to give the crude product TBM-4 that was used in the next step without further purification.

### Example S25. 1-(5-azido-4-(cyclopropylamino)pyridin-2-yl)-1H-pyrazolo[3,4-b]pyridine-5-carbonitrile (A-63)

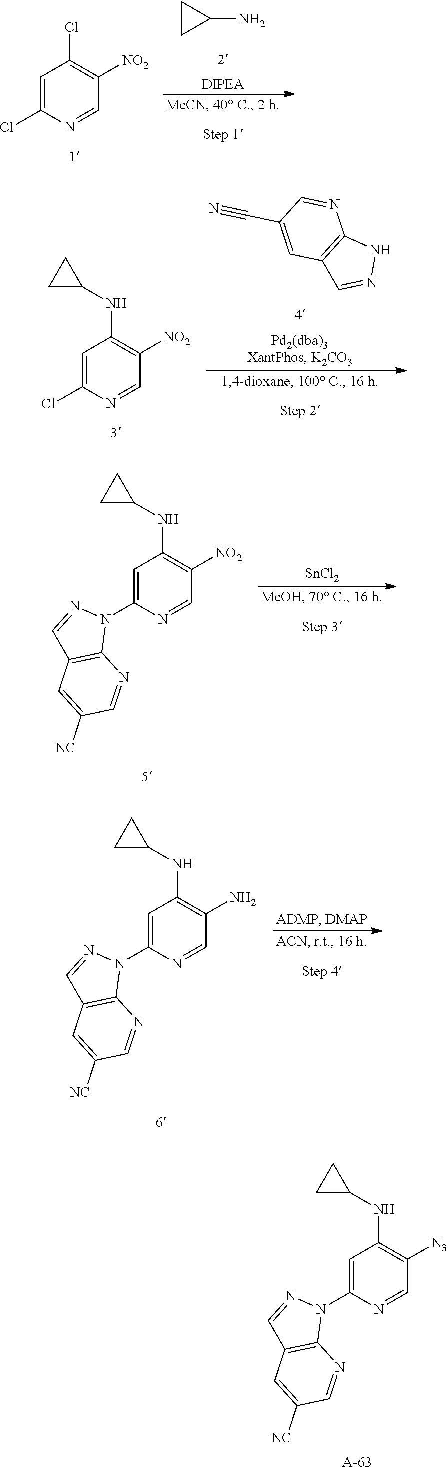

LCMS Method 1. Kinetex XB-C18, 75×3.0 mm, 2.6 μm, Temperature: RT, Flow: 1.0 mL/min, Run Time: 5 minutes, Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 100% Mobile Phase B for 4.0 min. MSD positive.

LCMS Method 2. X-Bridge C-18, 50×4.6 mm, 5.0 μm, Temperature: RT, Flow: 1.5 mL/min, Run Time: 5 minutes, Mobile Phase Conditions: Mobile Phase-A: 0.1% TFA in H₂O, Mobile Phase-B: 0.1% TFA in ACN, Gradient: Initial 95% Mobile Phase A and 5% Mobile Phase B linear gradient to 5% Mobile phase A and 95% Mobile Phase B for 2.5 min. MSD positive.

Step 1′. Preparation of 2-chloro-N-cyclopropyl-5-nitropyridin-4-amine 3′: To a solution of 2,4-dichloro-5-nitropyridine 1′ (4.0 g, 20.73 mmol) in acetonitrile (20.0 mL) was added cyclopropanamine 2′ (1.775 g, 31.1 mmol) and DIPEA (7.24 mL, 41.5 mmol) at RT, and the resulting solution was stirred at 40° C. for 2 h. The solution was then cooled to room temperature and concentrated under reduced pressure (70% volume). Finally, ice-cold water was added to the reaction mixture and stirred for 10 min to afford a pale yellow solid. The solid was filtered, washed with water, and dried to give the pure compound 2-chloro-N-cyclopropyl-5-nitropyridin-4-amine 3′ (4.0 g, 18.54 mmol, 89% yield) as a pale yellow solid.

LCMS method 1: retention time: 2.01 min, 99.32% purity at 254 nm, [M+H]⁺=214.0

¹H NMR (400 MHz, DMSO-d6): δ ppm 0.68-0.73 (m, 2H), 0.88-0.93 (m, 2H), 2.68-2.73 (m, 1H), 7.28 (s, 1H), 8.39 (brs, 1H), 8.89 (s, 1H).

Step 2′. Preparation of 1-(4-(cyclopropylamino)-5-nitropyridin-2-yl)-1H-pyrazolo[3,4-b]pyridine-5-carbonitrile 5′: To a solution of 2-chloro-N-cyclopropyl-5-nitropyridin-4-amine 3′ (1.0 g, 4.68 mmol) in 1,4-dioxane was added 1H-pyrazolo[3,4-b]pyridine-5-carbonitrile 4′ (0.810 g, 5.62 mmol), Xantphos (0.271 g, 0.468 mmol) and K₂CO₃ (1.294 g, 9.36 mmol), and the resulting mixture was purged with nitrogen gas for 10 min and then was added Pd₂dba₃ (0.214 g, 0.234 mmol). The resulting mixture was stirred at 100° C. for 16 h. The reaction mixture was cooled to RT and filtered through a celite bed, washed with ethyl acetate (3×50 mL), and concentrated under reduced pressure to give the crude product. The crude product was purified by flash column chromatography on silica gel (100-200 mesh) with 50-60% ethyl acetate to give pure 1-(4-(cyclopropylamino)-5-nitropyridin-2-yl)-1H-pyrazolo[3,4-b]pyridine-5-carbonitrile 5′ (700 mg, 1.80 mmol, 38.4% yield) as a pale yellow solid.

LCMS method 1: retention time 2.03 min, 82.64% purity at 220 nm, [M+H]⁺=322.0.

¹H NMR (400 MHz, DMSO-d6): δ ppm 0.77-0.81 (m, 2H), 0.94-0.99 (m, 2H), 2.73-2.76 (m, 1H), 8.18 (s, 1H), 8.50 (s, 1H), 8.74 (s, 1H), 9.05-9.06 (m, 1H), 9.13-9.14 (m, 2H).

Step 3′. Preparation of 1-(5-amino-4-(cyclopropylamino)pyridin-2-yl)-1H-pyrazolo[3,4-b]pyridine-5-carbonitrile 6′: To a stirred solution of 1-(4-(cyclopropylamino)-5-nitropyridin-2-yl)-1H-pyrazolo[3,4-b]pyridine-5-carbonitrile 5′ (500 mg, 1.556 mmol) in MeOH (10 mL) at RT was added tin(II) chloride (1.475 g, 7.78 mmol). The resulting solution was stirred at 70° C. for 16 h. The reaction mixture was cooled to RT and treated with ice-cold sat. sodium bicarbonate (50 mL) solution and extracted with 10% methanol in DCM (3×50 mL). The combined organic layer was dried over sodium sulphate and concentrated under reduced pressure to give the crude compound. The crude product was purified by flash column chromatography using silica gel (100-200 mesh) with 10% methanol/DCM to afford 1-(5-amino-4-(cyclopropylamino)pyridin-2-yl)-1H-pyrazolo[3,4-b]pyridine-5-carbonitrile 6′ (440 mg, 70.9% yield) as a brown solid.

LCMS method 2: retention time: 1.484 min, 73.0% purity at 220 nm, [M+H]⁺=292.1.

Step 4′. Preparation of 1-(5-azido-4-(cyclopropylamino)pyridin-2-yl)-1H-pyrazolo[3,4-b]pyridine-5-carbonitrile A-63: To a solution of 1-(5-amino-4-(cyclopropylamino) pyridin-2-yl)-1H-pyrazolo[3,4-b]pyridine-5-carbonitrile 6′ (440 mg, 1.51 mmol) in acetonitrile (5.0 mL) was added DMAP (227 mg, 2.266 mmol) and ADMP (646 mg, 2.266 mmol). The resulting solution was stirred for 16 h at RT. The reaction mixture was diluted with ice-cold water and extracted with ethyl acetate (3×20 mL). The combined organic layer was washed with brine, dried over sodium sulphate and then concentrated under reduced pressure to give the crude product 1-(5-azido-4-(cyclopropylamino)pyridin-2-yl)-1H-pyrazolo[3,4-b]pyridine-5-carbonitrile A-63 (500 mg, 34.8% yield) as a pale yellow liquid, which was used in the next step without further purification.

LCMS method 1: retention time: 1.877 min, [M+H]⁺=318.2.

### Example S26. 1-(5-azido-4-((1-cyanocyclopropyl) amino) pyridin-2-yl)-1H-pyrazolo[3,4-b]pyridine-5-carbonitrile (A-88)

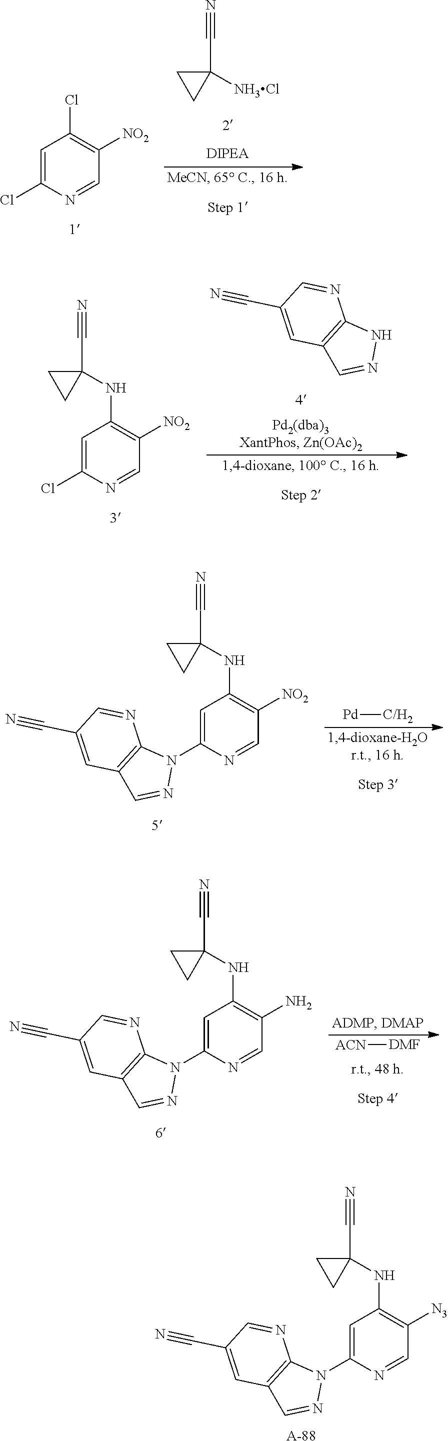

LCMS Method 1. Kinetex XB-C18, 75×3.0 mm, 2.6 Temperature: RT, Flow: 1.0 mL/min, Run Time: 5 minutes, Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 100% Mobile Phase B for 4.0 min. MSD positive.

LCMS Method 2. X-Bridge C-18, 50×4.6 mm, 5.0 μm, Temperature: RT, Flow: 1.0 mL/min, Run Time: 5 minutes, Mobile Phase Conditions: Mobile Phase-A: 0.1% TFA in H₂O, Mobile Phase-B: 0.1% TFA in ACN, Gradient: Initial 95% Mobile Phase A and 5% Mobile Phase B linear gradient to 5% Mobile phase A and 95% Mobile Phase B for 2.5 min. MSD positive.

UPLC Method 3. Aquity BEH C18, 50×3.0 mm, 1.7 Temperature: RT, Flow: 0.7 mL/min, Run Time: 2.5 minutes, Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 98% Mobile Phase B for 1.5 min. MSD positive.

Step 1′. Preparation of 14(2-chloro-5-nitropyridin-4-yl) amino) cyclopropane-1-carbonitrile 3′: To a suspension of 2,4-dichloro-5-nitropyridine 1′ (2.50 g, 12.95 mmol, 1.0 eq.) and 1-aminocyclopropane-1-carbonitrile HCl salt 2′ (3.07 g, 25.9 mmol, 2.0 eq.) in acetonitrile (25.0 mL, 0.51 M) under nitrogen was added DIPEA (13.58 mL, 78 mmol, 6.0 eq.) at RT. The resulting solution was stirred for 16 h at 65° C., cooled to room temperature, and poured into ice-cold water. The reaction mixture was stirred for 30 min to afford a pale brown solid. The solid was filtered, washed with water, and dried to give the pure compound 1-((2-chloro-5-nitropyridin-4-yl) amino) cyclopropane-1-carbonitrile 3′ (2.3 g, 71% yield) as a pale brown solid.

LCMS method 1: retention time 1.526 min, 95.83% purity at 220 nm, [M+H]⁺=239.0.

¹H NMR (400 MHz, DMSO-d6): δ ppm 1.46-1.50 (m, 2H), 1.82-1.85 (m, 2H), 7.22 (s, 1H), 8.42 (brs, 1H), 9.12 (s, 1H).

Step 2′. Preparation of 1-(4-((1-cyanocyclopropyl) amino)-5-nitropyridin-2-yl)-1H-pyrazolo[3,4-b]pyridine-5-carbonitrile 5′: A suspension of 1-((2-chloro-5-nitropyridin-4-yl) amino) cyclopropane-1-carbonitrile 3′ (2.50 g, 8.90 mmol, 1 eq.), 1H-pyrazolo[3,4-b]pyridine-5-carbonitrile 4′ (1.54 g, 10.69 mmol, 1.2 eq.), DIPEA (7.78 ml, 44.5 mmol, 5 eq.), and zinc acetate (0.654 g, 3.56 mmol, 0.4 eq.) in dioxane (25.0 mL, 0.356 M) was purged with nitrogen for 5 min. Xantphos (0.412 g, 0.712 mmol, 0.08 eq.) and Pd₂(dba)₃ (0.326 g, 0.356 mmol, 0.04 eq.) were added and the reaction mixture was degassed for another 15 min. The reaction mixture was stirred at 100° C. for 16 h in a sealed tube. The reaction mixture was cooled to RT and diluted with dioxane (15 mL), stirred for 5 min, and filtered. The obtained solid was then washed with dioxane (2×10 mL) and water (50 mL), and dried under vacuum to give crude 1-(4-((1-cyanocyclopropyl) amino)-5-nitropyridin-2-yl)-1H-pyrazolo[3,4-b]pyridine-5-carbonitrile 5′ (2.60 g) as an off-white solid, which was used in the next step without further purification.

LCMS method 1: retention time: 1.617 min, [M+H]⁺=347.0.

¹H NMR (400 MHz, DMSO-d6): δ ppm 1.55-1.59 (m, 2H), 1.85-1.87 (m, 2H), 8.34 (s, 1H), 8.80 (s, 1H), 9.08-9.22 (m, 4H).

Step 3′. Preparation of 1-(5-amino-4-((1-cyanocyclopropyl) amino) pyridin-2-yl)-1H-pyrazolo[3,4-b]pyridine-5-carbonitrile 6′: To a stirred solution of 1-(4-((1-cyanocyclopropyl) amino)-5-nitropyridin-2-yl)-1H-pyrazolo[3,4-b]pyridine-5-carbonitrile 5′ (2.6 g, 7.51 mmol, 1.0 eq) in dioxane:water (200 mL: 20 mL, 0.034 M) was added 10% Pd—C (600 mg) and stirred under hydrogen atmosphere for 16 h at RT. After completion of the reaction, the Pd—C catalyst was filtered off and the catalyst was washed with 1,4-dioxane (3×10 mL). The filtrate was concentrated under reduced pressure to give a crude product. The residue obtained was triturated with diethyl ether (25 mL) to give crude 1-(5-amino-4-((1-cyanocyclopropyl) amino) pyridin-2-yl)-1H-pyrazolo[3,4-b]pyridine-5-carbonitrile 6′ (2.35 g, 82% yield) as a yellow solid, which was used in the next step without further purification.

LCMS method 2: retention time: 1.805 min, 83% purity at 220 nm, [M+H]⁺=317.2.

Step 4′. Preparation of 1-(5-azido-4-((1-cyanocyclopropyl) amino) pyridin-2-yl)-1H-pyrazolo[3,4-b]pyridine-5-carbonitrile A-88: To a suspension of 1-(5-amino-4-((1-cyanocyclopropyl) amino) pyridin-2-yl)-1H-pyrazolo[3,4-b]pyridine-5-carbonitrile 6′ (2.3 g, 5.30 mmol, 1 eq.) in acetonitrile:DMF (25 mL:10 mL) was added DMAP (1.295 g, 10.60 mmol, 2 eq.) at RT. 2-Amino-4,6-dimethoxypyrimidine (ADMP) (1.645 g, 10.60 mmol, 2 eq.) was then added and allowed to stir for 48 h at RT (0.5 eq. of extra ADMP was added to complete the reaction after 24 h). The reaction mixture was poured into ice-cold water and was stirred for 30 min to afford a solid. The solid was filtered, washed with water, and dried to give the crude 1-(5-azido-4-((1-cyanocyclopropyl) amino) pyridin-2-yl)-1H-pyrazolo[3,4-b]pyridine-5-carbonitrile A-88 (2.01 g, 4.43 mmol, 84% yield) as a dark grey solid, which was used in the next step without further purification.

UPLC method 3: retention time: 1.054 min, 75.45% purity at 220 nm, [M+H]⁺=343.0.

TBM intermediates prepared using General Procedure TBM-4 are summarized in Table 17.

TABLE 17
TBM Intermediates Prepared via General Procedure TBM-4

| | Structure | Heterobicycle | Amine |
|---|---|---|---|
| A-13 | 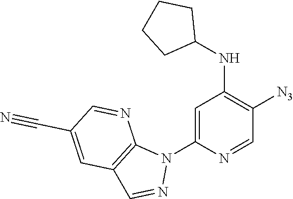 [SMILES: N#Cc1cnc2c(cnn2-c2cc(NC3CCCC3)c(N=[N+]=[N-])cn2)c1] | 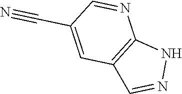 [SMILES: N#Cc1cnc2[nH]ncc2c1] | 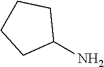 |
| A-63 | 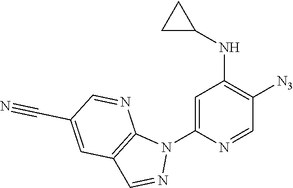 [SMILES: N#Cc1cnc2c(cnn2-c2cc(NC3CC3)c(N=[N+]=[N-])cn2)c1] | 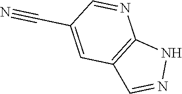 [SMILES: N#Cc1cnc2[nH]ncc2c1] | 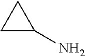 |
| A-70 | 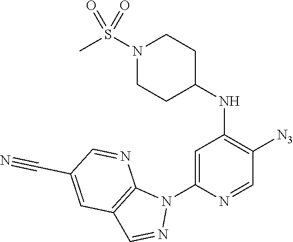 [SMILES: CS(=O)(=O)N1CCC(Nc2cc(-n3ncc4cc(C#N)cnc43)ncc2N=[N+]=[N-])CC1] | 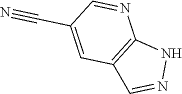 [SMILES: N#Cc1cnc2[nH]ncc2c1] | 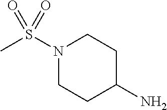 |
| A-88 | 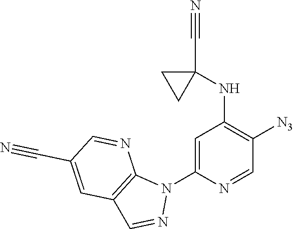 [SMILES: N#Cc1cnc2c(cnn2-c2cc(NC3(C#N)CC3)c(N=[N+]=[N-])cn2)c1] | 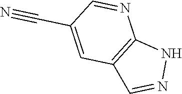 [SMILES: N#Cc1cnc2[nH]ncc2c1] | 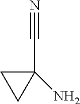 |
| A-58 | 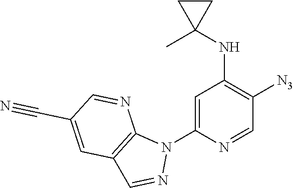 [SMILES: CC1(Nc2cc(-n3ncc4cc(C#N)cnc43)ncc2N=[N+]=[N-])CC1] | 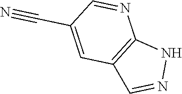 [SMILES: N#Cc1cnc2[nH]ncc2c1] | 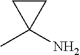 |
| A-69 | 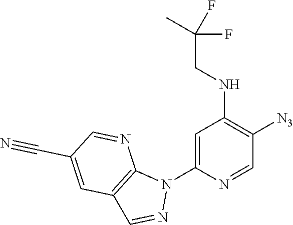 [SMILES: CC(F)(F)CNc1cc(-n2ncc3cc(C#N)cnc32)ncc1N=[N+]=[N-]] | 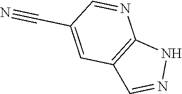 [SMILES: N#Cc1cnc2[nH]ncc2c1] | 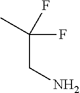 |
| A-71 | 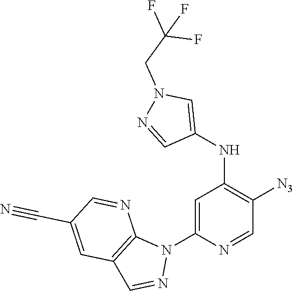 [SMILES: N#Cc1cnc2c(cnn2-c2cc(Nc3cnn(CC(F)(F)F)c3)c(N=[N+]=[N-])cn2)c1] | 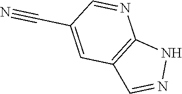 [SMILES: N#Cc1cnc2[nH]ncc2c1] | 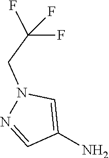 [SMILES: Nc1cnn(CC(F)(F)F)c1] |

### General Procedure TBM-5

### Example S27. 6-(6-Cyanopyrazolo[1,5-a]pyrimidin-3-yl)-4-(cyclopropylamino)pyridine-3-carboxylic acid (A-31)

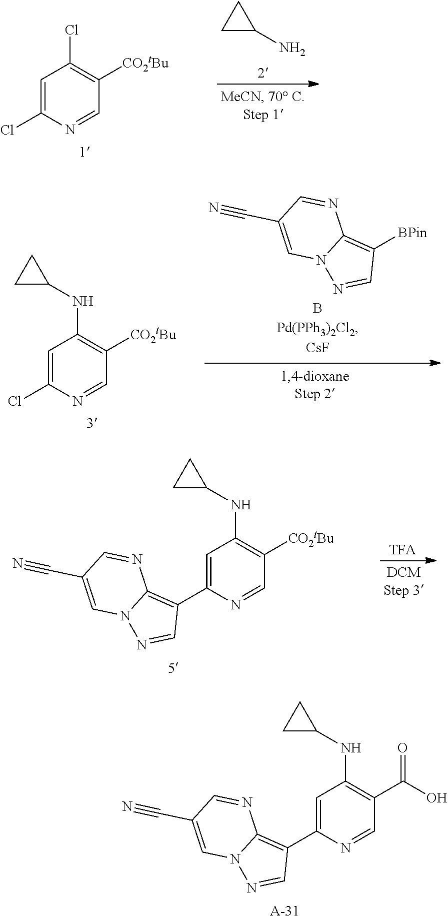

LCMS Method 1. Column: Luna C18 (2) 50×3 mm, 3 um. Temperature: 45° C., Flow: 1.5 mL/min, run time: 2.5 min. Mobile phase conditions: Initial 95% H₂O 0.1% FA/5% MeCN 0.1% FA, linear gradient to 95% MeCN 0.1% FA over 1.3 min then hold for 1.2 minute at 95% MeCN 0.1% FA. MSD: ESI Positive

### Preparation of 3-(4,4,5,5-Tetramethyl-1,3,2-dioxaborolan-2-yl)pyrazolo[1,5-a]pyrimidine-6-carbonitrile (B)

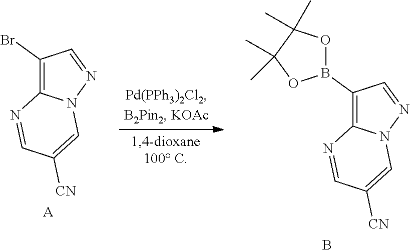

A flame-dried microwave tube equipped with a stir bar was loaded with KOAc (264 mg, 2.69 mmol, 2.0 eq.), B₂pin₂ (1366 mg, 5.38 mmol, 4.0 eq.), A (300 mg, 1.35 mmol, 1.0 eq.) and Pd(PPh₃)₂Cl₂ (47.21 mg, 0.070 mmol, 5 mol %). The vial was purged with nitrogen for 10 minutes, then 1,4-dioxane (6.72 mL, 0.2 M) was added. Nitrogen was sparged through the solution for another 10 minutes. The microwave tube was sealed and placed in a preheated aluminium block. The reaction mixture was stirred at 100° C. for 16 h. After 16 h, the reaction mixture was filtered on Celite, rinsing with pentane. The filtrate was concentrated under reduced pressure. The residue was triturated in pentane and then centrifuged (2×) which afforded B (235 mg, 61% yield) as a brown solid.

LCMS method 1: retention time: 1.635 min, 80.8% purity at 215 nm, [M+H]⁺=271.1.

¹H NMR (400 MHz, DMSO-d6) δ ppm 1.39 (s, 12H), 8.57 (s, 1H), 8.74 (d, J=2.1 Hz, 1H), 9.09 (d, J=2.1 Hz, 1H).

Step 1′. Preparation of ethyl 6-chloro-4-(cyclopropylamino)pyridine-3-carboxylate (3′): A 250 mL round bottom flask was loaded with tert-butyl 4,6-dichloropyridine-3-carboxylate 1′ (1.5 g, 6.82 mmol, 1 eq.), MeCN (50 mL) and cyclopropanamine 2′ (1.26 mL, 20.45 mmol, 3 eq.) and the mixture was stirred at 70° C. After 18 h, LCMS showed complete conversion. Volatiles were removed under vacuum and the residue was purified by normal phase flash chromatography (40 g silica column; elution: 0% EtOAc/Heptane for 3 CVs, 0% to 20% EtOAc/Heptane over 13 CVs). Fractions were collected and concentrated to give 3′ (1.5 g, 6.232 mmol, 92% yield) as a white solid.

LCMS method 1: 99.9% purity at 215 nm, [M+H]⁺=241.2.

¹H NMR (400 MHz, CDCl₃) δ ppm 0.61-0.66 (m, 2H), 0.88-0.94 (m, 2H), 1.39-1.42 (t, J=7.2 Hz, 3H), 2.47-2.55 (m, 1H), 4.35 (q, J=7.1 Hz, 2H), 6.98 (s, 1H), 8.23 (br s, 1H), 8.68 (s, 1H).

Step 2′. Preparation of tert-Butyl 6-(6-cyanopyrazolo[1,5-a]pyrimidin-3-yl)-4-(cyclopropylamino)pyridine-3-carboxylate (5′): A solution of 3′ (230 mg, 0.850 mmol, 1.5 eq.), 4′ (152.57 mg, 0.570 mmol, 1.0 eq.) and CsF (258.8 mg, 1.7 mmol, 3.0 eq.) in 1,4-dioxane (1.70 mL, 0.28 M) and water (0.312 mL, 0.28 M) was degassed by bubbling nitrogen under sonication for 5 minutes. Pd(PPh₃)₂Cl₂ (39.8 mg, 0.060 mmol, 10 mol %) was added and the solution was degassed a second time by bubbling nitrogen under sonication for 5 minutes. The resulting mixture was stirred for 70 minutes at 120° C. under microwave irradiation. After 70 minutes, the reaction mixture was diluted with EtOAc. The organics were washed with brine (2×), dried over Na₂SO₄ and concentrated to dryness. The crude mixture was purified by normal phase flash chromatography (heptanes/EtOAc). The desired fractions were concentrated to dryness to afford 5′ (90 mg, 41% yield) as a yellow solid.

LCMS method 1: retention time: 1.503 min, 99.9% purity at 215 nm, [M+H]⁺=377.1.

¹H NMR (400 MHz, CDCl₃) δ ppm 0.64-0.70 (m, 2H), 0.91-0.96 (m, 2H), 1.24 (s, 9H), 2.60-2.67 (m, 1H), 8.11 (s, 1H), 8.22 (br s, 1H), 8.69 (d, J=2.26 Hz, 1H), 8.90 (s, 1H), 9.04 (s, 1H), 9.09 (d, J=2.13 Hz, 1H).

Step 3′. Preparation of 6-(6-Cyanopyrazolo[1,5-a]pyrimidin-3-yl)-4-(cyclopropylamino)pyridine-3-carboxylic acid (A-31): Trifluoroacetic acid (1.0 mL, 13.16 mmol, 83 eq.) was added to a solution of 5′ (60.0 mg, 0.160 mmol, 1.0 eq.) in DCM (0.50 mL, 0.31 M). The mixture was stirred at room temperature for 6 h with monitoring by LCMS. After 6 h, the solution was concentrated to dryness and co-evaporated with toluene and MeCN which afforded A-31 (40 mg, 79% yield) as a yellow film. Used as-is for next step.

LCMS method 1: retention time: 1.503 min, 57.6% purity at 215 nm, [M+H]⁺=321.1.

### Example S28. 6-(3-cyanopyrrolo[1,2-b]pyridazin-7-yl)-4-(cyclopropylamino)pyridine-3-carboxylic acid (A-40)

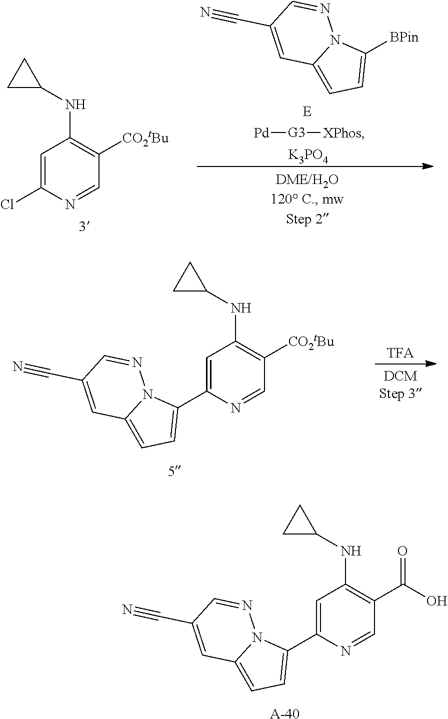

LCMS Method 1. Column: Luna C18 (2) 50×3 mm, 3 um. Temperature: 45° C., Flow: 1.5 mL/min, run time: 2.5 min. Mobile phase conditions: Initial 95% H₂O 0.1% FA/5% MeCN 0.1% FA, linear gradient to 95% MeCN 0.1% FA over 1.3 min then hold for 1.2 minute at 95 MeCN 0.1% FA. MSD: ESI Positive

### Synthesis of 7-(4,4,5,5-tetramethyl-1,3,2-dioxaborolan-2-yl)pyrrolo[1,2-b]pyridazine-3-carbonitrile (E)

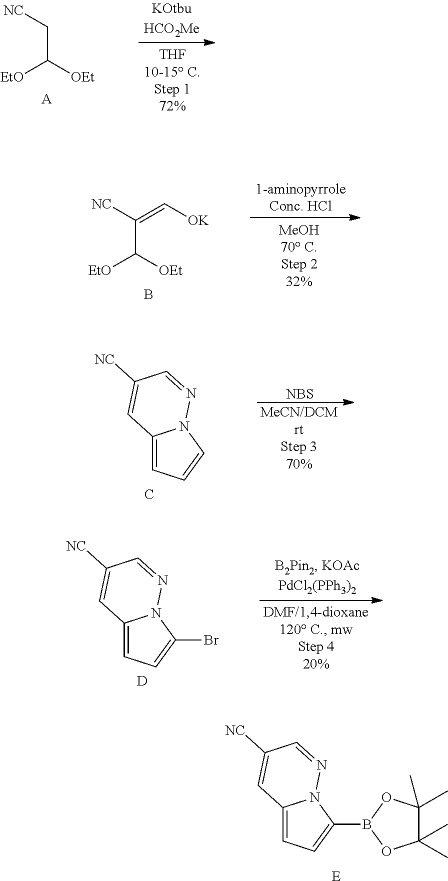

Step 1. Preparation of 3,3-diethoxy-2-formylpropionitrile potassium salt (B): A 1 M solution of KOtbu in THF (38 ml, 38 mmol) was added to a solution of 3,3-diethoxypropanenitrile A (5.24 mL, 34.92 mmol) and methyl formate (2.67 mL, 43.65 mmol) in THF (19.0 mL) over a period of 45 min while the internal temperature was maintained at 10-15° C. The reaction mixture was stirred for 2 h at RT after addition. Then, heptanes (1 ml) was added and the stirring continued for 20 min. The slurry filtered and the filter cake was washed with 1/1 heptanes/THF and dried overnight to afford 3,3-diethoxy-2-formylpropionitrile potassium salt B (5.28 g, 72% yield) as an off-white solid.

Step 2. Preparation of pyrrolo[1,2-b]pyridazine-3-carbonitrile (C): Concentrated HCl (13.6 mL, 147.42 mmol) was slowly added to a suspension of 3,3-diethoxy-2-formylpropionitrile potassium salt B (9.8 g, 46.83 mmol) in MeOH (80 mL) so that the temperature does not exceed 20° C. and the mixture was stirred at room temperature for 20 min. Then, a solution of 1-aminopyrrole (1.73 g, 21.06 mmol) in MeOH (20 mL) was added and the mixture was heated at reflux for another 2 hours. The reaction mixture was cooled to room temperature, neutralized by addition of saturated aqueous NaHCO₃ solution and then concentrated under reduced pressure to ⅓ its original volume. The mixture was extracted to MTBE three times, and the combined organic phases were dried over Na₂SO₄, filtered and concentrated under reduced pressure. The residue was purified by silica gel column chromatography (EtOAc/heptane) to give pyrrolo[1,2-b]pyridazine-3-carbonitrile C (0.98 g, 32% yield) as a yellow solid.

LCMS method 1: 99.9% purity at 215 nm, [M+H]⁺=144.1.

¹H NMR (400 MHz, CDCl₃) δ ppm 6.85 (dd, J=4.6, 1.0 Hz, 1H), 7.05 (dd, J=4.4, 2.7 Hz, 1H), 7.94 (s, 1H), 8.10-8.16 (m, 2H).

Step 3. Preparation of 7-bromopyrrolo[1,2-b]pyridazine-3-carbonitrile (D): To a solution of pyrrolo[1,2-b]pyridazine-3-carbonitrile C (416 mg, 2.91 mmol) in DCM (15.0 mL) and MeCN (7.5 mL) was added NBS (371 mg, 3.49 mmol) and the mixture was stirred at rt. After 18 h, the reaction was quenched by the addition of saturated aqueous NaHCO₃ solution. The mixture was extracted with DCM two more times and the combined organic phases were dried over Na₂SO₄, filtered and concentrated under reduced pressure. The residue was purified by reversed phase C18 column chromatography (MeOH/0.1% formic acid in water) to afford 7-bromopyrrolo[1,2-b]pyridazine-3-carbonitrile D (526 mg, 70% yield) as a yellow solid.

LCMS method 1: 75% purity at 215 nm, [M+H]⁺=222.2, 224.2.

¹H NMR (400 MHz, CDCl₃) δ ppm 6.93 (d, J=4.6 Hz, 1H), 7.12 (d, J=4.9 Hz, 1H), 8.10 (d, J=1.7 Hz, 1H), 8.28 (d, J=1.7 Hz, 1H).

Step 4. Preparation of 7-(4,4,5,5-tetramethyl-1,3,2-dioxaborolan-2-yl)pyrrolo[1,2-b]pyridazine-3-carbonitrile (E): A microwave vial was charged with 7-bromopyrrolo[1,2-b]pyridazine-3-carbonitrile (225 mg, 0.88 mmol), B₂pin₂ (672 mg, 2.65 mmol) and potassium acetate (260 mg, 2.65 mmol) followed by 1,4-dioxane (4.0 mL) and DMF (1.0 mL). The solution was degassed with N₂ for 5 min. Pd(PPh₃)₂Cl₂ (619.86 mg, 0.8800 mmol) was added and degassing continued for 5 min. The vial was sealed and heated in a microwave reactor at 120° C. for 1 h. The mixture was filtered through Celite and the filter cake was further washed with MTBE. The filtrate was washed with water and the aqueous phase was back extracted to MTBE 2×. The combined organics were washed with brine and dried over sodium sulfate. The solvent was removed under reduced pressure. Purification by silica gel column chromatography (EtOAc/heptanes) afforded 7-(4,4,5,5-tetramethyl-1,3,2-dioxaborolan-2-yl)pyrrolo[1,2-b]pyridazine-3-carbonitrile (70 mg, 19% yield) as a yellow solid.

LCMS method 1: 99.9% purity at 215 nm, [M-C₆H₁₀+H]⁺=188.2.

¹H NMR (400 MHz, MeOH-d4) δ ppm 1.38 (s, 12H), 6.92 (d, J=4.4 Hz, 1H), 7.45 (d, J=4.4 Hz, 1H), 8.33 (d, J=2.2 Hz, 1H), 8.48 (d, J=2.2 Hz, 1H).

Step 2″. Preparation of tert-butyl 6-(3-cyanopyrrolo[1,2-b]pyridazin-7-yl)-4-(cyclopropylamino)pyridine-3-carboxylate (5″): To a solution of tert-butyl 6-chloro-4-(cyclopropylamino)pyridine-3-carboxylate 3′ (82 mg, 0.31 mmol), 7-(4,4,5,5-tetramethyl-1,3,2-dioxaborolan-2-yl)pyrrolo[1,2-b]pyridazine-3-carbonitrile E (100 mg, 0.37 mmol) in DME (1.4 mL) was added a solution of K₃PO₄ (129.54 mg, 0.6100 mmol) in water (1.0 mL). The resulting solution was degassed by bubbling nitrogen for 5 min, then Pd G3 XPhos (25 mg, 0.03 mmol) was added and the solution was degassed for another 5 min. The reaction mixture was stirred for minutes at 120° C. under microwave irradiation then was diluted with EtOAc and water. The phases were separated and the organic layer was washed with water, then brine, dried over Na₂SO₄ and concentrated to dryness. The crude mixture was purified by silica gel column chromatography (EtOAc/heptanes) to afford tert-butyl 6-(3-cyanopyrrolo[1,2-b]pyridazin-7-yl)-4-(cyclopropylamino)pyridine-3-carboxylate 5″ (90 mg, 74% yield) as an orange solid.

LCMS method 1: 99.9% purity at 215 nm, [M+2H]²⁺=376.2.

¹H NMR (400 MHz, CDCl₃) δ ppm 0.67-0.74 (m, 2H), 0.82-0.90 (m, 1H), 0.91-0.98 (m, 2H), 1.60 (s, 9H), 6.99 (d, J=4.9 Hz, 1H), 7.94 (d, J=4.9 Hz, 1H), 8.20 (d, J=2.0 Hz, 1H), 8.24-8.28 (m, 1H), 8.33 (d, J=2.0 Hz, 1H), 8.49 (s, 1H), 8.95 (s, 1H).

Step 3″. Preparation of 6-(3-cyanopyrrolo[1,2-b]pyridazin-7-yl)-4-(cyclopropylamino)pyridine-3-carboxylic acid (A-40): To a solution of tert-butyl 6-(3-cyanopyrrolo[1,2-b]pyridazin-7-yl)-4-(cyclopropylamino)pyridine-3-carboxylate 5″ (85 mg, mmol) in DCM (3.0 mL) was added TFA (3.81 mL, 14.92 mmol) at rt. After 16 h stirring at RT, the volatiles were removed under reduced pressure and the residue was co-evaporated with MeCN 3× and precipitated in EtOAc to afford 6-(3-cyanopyrrolo[1,2-b]pyridazin-7-yl)-4-(cyclopropylamino)pyridine-3-carboxylic acid (60 mg, 61% yield) as a yellow solid.

LCMS method 1: 99.9% purity at 215 nm, [M+2H]²⁺=320.2.

¹H NMR (400 MHz, DMSO-d6) δ ppm 0.65-0.79 (m, 2H), 0.96-1.09 (m, 2H), 2.76-2.84 (m, 1H), 7.22 (d, J=4.9 Hz, 1H), 8.03 (d, J=4.9 Hz, 1H), 8.46 (s, 1H), 8.78 (s, 1H), 8.88 (d, J=2.2 Hz, 1H).

TBM intermediates prepared using General Procedure TBM-5 are summarized in Table 18.

TABLE 18
TBM Intermediates Prepared via General Procedure TBM-5

| | Structure | Heterobicycle | Amine |
|---|---|---|---|
| A-23 | 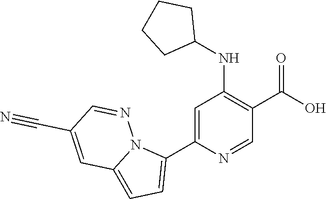 [SMILES: N#Cc1cnn2c(-c3cc(NC4CCCC4)c(C(=O)O)cn3)ccc2c1] | 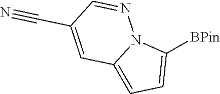 | 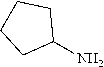 |
| A-40 | 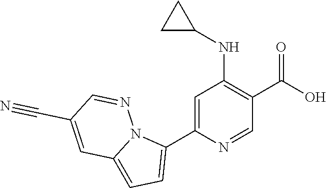 [SMILES: N#Cc1cnn2c(-c3cc(NC4CC4)c(C(=O)O)cn3)ccc2c1] | 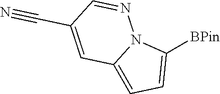 | 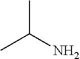 |
| A-31 | 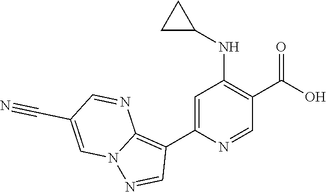 [SMILES: N#Cc1cnc2c(-c3cc(NC4CC4)c(C(=O)O)cn3)cnn2c1] | 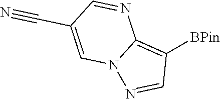 | 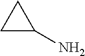 |
| A-49 | 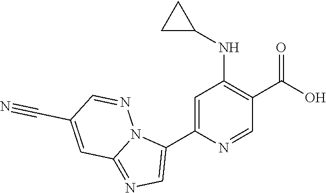 [SMILES: N#Cc1cnn2c(-c3cc(NC4CC4)c(C(=O)O)cn3)cnc2c1] | 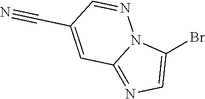 [SMILES: N#Cc1cnn2c(Br)cnc2c1] | 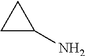 |

### General Procedure TBM-6

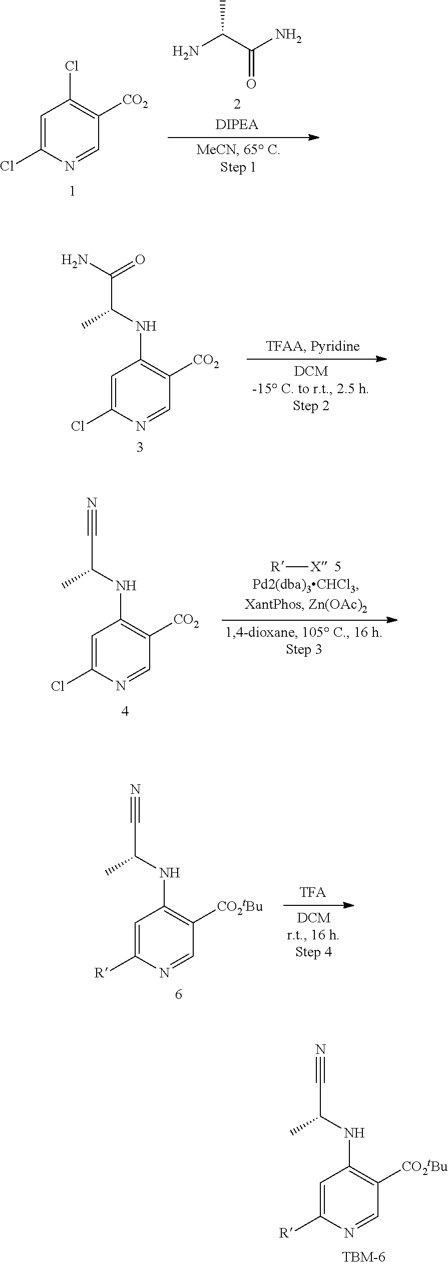

wherein R′ is a bicyclic heteroaryl and X″ is a chemical moiety capable of undergoing Pd-catalyzed coupling.

Step 1. Preparation of Tert-butyl 6-chloro-4-[[(1R)-2-amino-1-methyl-2-oxo-ethyl]amino]pyridine-3-carboxylate 3: To a solution of (2R)-2-aminopropanamide hydrochloride 2 (3.0 eq.) and tert-butyl 4,6-dichloropyridine-3-carboxylate 1 (1.0 eq.) in MeCN (0.2 M) under nitrogen was added DIPEA (4.0 eq.). The reaction was stirred at 65° C. for 96 hours. The reaction mixture was cooled down to room temperature. Solvent was removed under high vacuum. DCM and water were added, then phases were separated. The aqueous phase was extracted with DCM. The combined organic phases were washed once with brine, dried over magnesium sulfate, filtered and concentrated. The residue was purified by silical gel chromatography. Fractions were combined and concentrated to give product 3.

Step 2′. Preparation of tert-butyl 6-chloro-4-[[(1R)-1-cyanoethyl]amino]pyridine-3-carboxylate 4′: To a solution of 3 (1.0 eq.) and pyridine (10.0 eq.) in DCM (0.2 M) was added TFAA (2.0 eq.) in DCM (0.1 M) dropwise at −15° C. The resulting mixture was allowed to warm to room temperature and stirred for 2.5 hours. The mixture was washed with 1 M HCl, saturated sodium bicarbonate and brine. The organic layer was dried, filtered and concentrated. The residue was purified by normal phase flash chromatography. Fractions were combined and concentrated to give product 4.

Step 3′. Preparation of tert-butyl 6-(5-cyanopyrazolo[3,4-b]pyridin-1-yl)-4-[[(1R)-1-cyanoethyl]amino]pyridine-3-carboxylate 4: To a dried sealed tube was added 4 (1.0 eq.), coupling partner 5 (1.0 eq.), XantPhos (0.15 eq.), Pd₂(dba)₃·CHCl₃ (0.15 eq.), and Zn(OAc)₂ (1.0 eq.) in 1,4-dioxane (0.3 M). The mixture was sparged with nitrogen before the tube was sealed and the mixture was stirred at at heat. The completed reaction was filtered over celite, washed with DCM and the filtrate was evaporated. The residue was purified by normal phase flash chromatography. Fractions were combined and concentrated under reduce pressure to afford 6.

Similar C—N or C—C coupling reactions can be used in this transformation.

Step 4′. Preparation of 6-(5-Cyanopyrazolo[3,4-b]pyridin-1-yl)-4-[[(1R)-1-cyanoethyl]amino]pyridine-3-carboxylic acid A-29: To a solution of 6 (1.0 eq.) in DCM (0.3 M) was added TFA (15 eq.). The reaction was stirred at room temperature for 16 hours. The solvent was removed in vacuo and the residue was co-evaporated with PhMe and MeCN prior to Afforded a yellow semi-solid to which was added H₂O (10 mL)/MeCN (2 mL)/EtOAc (2 mL) and the mixture was sonicated 3-5 minutes. Solvent evaporated almost until dry, a precipitate formed which was filtered, washed with MTBE, which afforded 182 mg of A-29. The analysis of remaining solution shows a mixture of A-29 and by-products. Purified by reverse-phase chromatography column using a. The pure fractions were combined and concentrated under reduced pressure to afford carboxylic acid TBM-6.

### Example S29. 6-(5-Cyanopyrazolo[3,4-b]pyridin-1-yl)-4-[[(1R)-1-cyanoethyl]amino]pyridine-3-carboxylic acid (A-29)

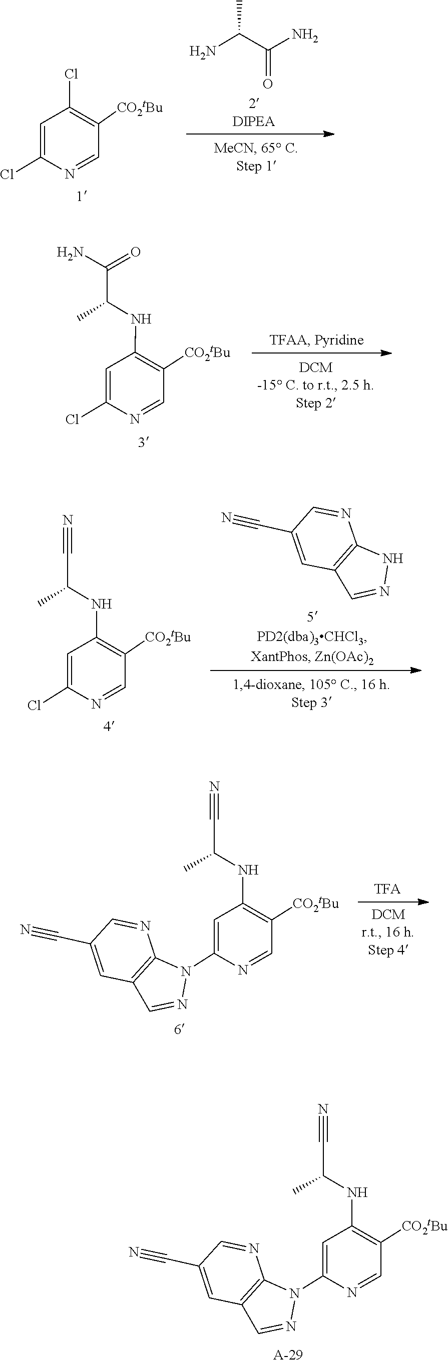

LCMS Method 1. Column: Luna C18 (2) 50×3 mm, 3 um. Temperature: 45° C., Flow: 1.5 mL/min, run time: 2.5 min. Mobile phase conditions: Initial 95% H₂O 0.1% FA/5% MeCN 0.1% FA, linear gradient to 95% MeCN 0.1% FA over 1.3 min then hold for 1.2 minute at 95 MeCN 0.1% FA. MSD: ESI Positive

Step 1′. Preparation of Tert-butyl 6-chloro-4-[[(1R)-2-amino-1-methyl-2-oxo-ethyl]amino]pyridine-3-carboxylate 3′: To a solution of (2R)-2-aminopropanamide hydrochloride 2′ (1.5 g, 12.091 mmol, 3.0 eq.) and tert-butyl 4,6-dichloropyridine-3-carboxylate 1′ (1.0 g, 4.030 mmol, 1.0 eq.) in MeCN (20 mL, 0.2M) under nitrogen was added DIPEA (2.81 mL, 16.122 mmol, 4.0 eq.). The reaction was stirred at 65° C. for 96 hours. The reaction mixture was cooled down to room temperature. Acetonitrile was removed under high vacuum. DCM and water were added, then phases were separated. The aqueous phase was extracted 3 times with DCM. The combined organic phases were washed once with brine, dried over magnesium sulfate, filtered and concentrated. The residue was purified by normal phase flash chromatography (40 g silica column, elution: 0 to 10% CH₂Cl₂/CH₃OH over 15 CV, product exited at 5% CH₃OH). Fractions were combined and concentrated to give tert-butyl 6-chloro-4-[[(1R)-2-amino-1-methyl-2-oxo-ethyl]amino]pyridine-3-carboxylate 3′ (1 g, 83% yield) as a white solid.

LCMS method 1: retention time: 1.601 min, 99.9% purity at 215 nm, [M+H]⁺=300.2.

¹H NMR (400 MHz, DMSO-d6) δ ppm 1.36 (d, J=6.8 Hz, 3H), 1.55 (s, 9H), 4.15 (t, J=6.8 Hz, 1H), 6.57 (s, 1H), 7.31 (br s, 1H), 7.62 (br s, 1H), 8.44 (d, J=6.8 Hz, 1H), 8.50 (s, 1H).

Step 2′. Preparation of tert-butyl 6-chloro-4-[[(1R)-1-cyanoethyl]amino]pyridine-3-carboxylate 4′: To a solution of tert-butyl 6-chloro-4-[[(1R)-2-amino-1-methyl-2-oxo-ethyl]amino]pyridine-3-carboxylate 3′ (1.0 g, 3.336 mmol, 1.0 eq.) and pyridine (2.7 mL, 33.361 mmol, 10.0 eq.) in DCM (19.0 mL) was added TFAA (0.9 mL, 6.672 mmol, 2.0 eq.) in DCM (11.4 mL, 0.1 M) dropwise at −15° C. The resulting mixture was allowed to warm to room temperature and stirred for 2.5 hours. The mixture was washed with 1 M HCl (3×30 mL), saturated sodium bicarbonate (1×40 mL) and brine (1×40 mL). The organic layer was dried over Na₂SO₄, filtered and concentrated. The residue was purified by normal phase flash chromatography (40 g silica column, elution: 0 to 5% CH₂Cl₂/CH₃OH over 15 CV, product exited at 1.0% CH₃OH). Fractions were combined and concentrated to give tert-butyl 6-chloro-4-[[(1R)-1-cyanoethyl]amino]pyridine-3-carboxylate 4′ (0.76 g, 84% yield) as a white solid.

LCMS method 1: retention time: 1.833 min, 98.7% purity at 215 nm, [M+H]⁺=282.2.

¹H NMR (400 MHz, DMSO-d6) δ ppm 1.56 (s, 9H), 1.66 (d, J=7.1 Hz, 3H), 5.01 (quin, J=7.1 Hz, 1H), 7.06 (s, 1H), 8.19 (d, J=7.6 Hz, 1H), 8.59 (s, 1H).

Step 3′. Preparation of tert-butyl 6-(5-cyanopyrazolo[3,4-b]pyridin-1-yl)-4-[[(1R)-1-cyanoethyl]amino]pyridine-3-carboxylate 4: To a dried sealed tube was added tert-butyl 6-chloro-4-[[(1R)-1-cyanoethyl]amino]pyridine-3-carboxylate 4′ (760.0 mg, 2.697 mmol, 1.0 eq.), 1H-pyrazolo[3,4-b]pyridine-5-carbonitrile 5′ (388.8 mg, 2.697 mmol, 1.0 eq.), XantPhos (0.234 g, 0.404 mmol, 0.15 eq.), Pd₂(dba)₃·CHCl₃ (418.8 mg, 0.404 mmol, 0.15 eq.), Zn(OAc)₂ (494.9 mg, 2.697 mmol, 1.0 eq.) in 1,4-dioxane (9.0 mL, 0.3 M). Then nitrogen was sparged through the mixture for 15 minutes. The tube was sealed and the mixture was stirred at 105° C. for 16 hours. The reaction was filtered over celite, washed with DCM and the filtrate was evaporated. The residue was purified by normal phase flash chromatography (40 g silica column, elution: 0 to 10% DCM/MeOH over 15 CV, product exited at 5% MeOH). Fractions from the major peak were combined and concentrated under reduce pressure to afford tert-butyl 6-(5-cyanopyrazolo[3,4-b]pyridin-1-yl)-4-[[(1R)-1-cyanoethyl]amino]pyridine-3-carboxylate 6′ (906 mg, 77% yield) as a orange solid. Contaminated with XantPhosO₂.

LCMS method 1: retention time: 1.781 min, 88% purity at 215 nm, [M+H]⁺=390.2.

¹H NMR (400 MHz, DMSO-d6) δ ppm 1.60 (s, 9H), 1.72 (br d, J=6.8 Hz, 3H), 4.99-5.10 (m, 1H), 7.60 (s, 1H), 8.30 (br d, J=7.1 Hz, 1H), 8.71 (s, 1H), 8.87 (s, 1H), 9.05 (dd, J=12.1, 1.8 Hz, 1H), 11.96 (br s, 1H).

Step 4′. Preparation of 6-(5-Cyanopyrazolo[3,4-b]pyridin-1-yl)-4-[[(1R)-1-cyanoethyl]amino]pyridine-3-carboxylic acid A-29: To a solution of tert-butyl 6-(5-cyanopyrazolo[3,4-b]pyridin-1-yl)-4-[[(1R)-1-cyanoethyl]amino]pyridine-3-carboxylate 6′ (1.17 g, 3.025 mmol, 1.0 eq.) in DCM (8.9 mL, 0.3 M) was added TFA (11.6 mL, 45.376 mmol, 15 eq.). The reaction was stirred at room temperature for 16 hours. The solvent was removed in vacuo and the residue was co-evaporated with PhMe (3 times) followed by MeCN (2 times), which afforded a yellow semi-solid to which was added H₂O (10 mL)/MeCN (2 mL)/EtOAc (2 mL) and the mixture was sonicated 3-5 minutes. Solvent evaporated almost until dry, a precipitate formed which was filtered, washed with MTBE, which afforded 182 mg of A-29. The analysis of remaining solution shows a mixture of A-29 and by-products. Purified by reverse-phase chromatography column using a 50G C18 column (elution: 5% MeOH/0.1% HCOOH over 4 CV, then 5% to 75% MeOH/0.1% HCOOH over 15 CV, product exited at 48% MeOH). The pure fractions were combined and concentrated under reduced pressure to afford 6-(5-cyanopyrazolo[3,4-b]pyridin-1-yl)-4-[[(1R)-1-cyanoethyl]amino]pyridine-3-carboxylic acid A-29 (315 mg, 29% yield) as a white solid.

LCMS method 1: retention time: 1.372 min, 99.9% purity at 215 nm, [M+H]⁺=334.2.

¹H NMR (400 MHz, DMSO-d6) δ ppm 1.71 (d, J=6.8 Hz, 3H), 4.99-5.10 (m, 1H), 7.59 (s, 1H), 8.55 (br d, J=7.6 Hz, 1H), 8.72 (s, 1H), 8.88 (s, 1H), 9.04 (d, J=2.0 Hz, 1H), 9.07 (d, J=2.0 Hz, 1H), 13.63 (br s, 1H).

TBM intermediates prepared using General Procedure TBM-6 are summarized in Table 19.

TABLE 19
TBM Intermediates Prepared via General Procedure TBM-6

| | Structure | Heterobicycle |
|---|---|---|
| A-29 | 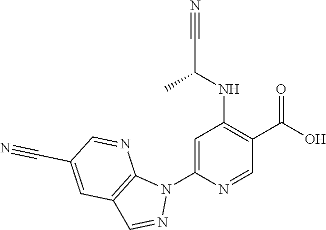 [SMILES: C[C@H](C#N)Nc1cc(-n2ncc3cc(C#N)cnc32)ncc1C(=O)O] | 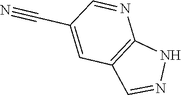 [SMILES: N#Cc1cnc2[nH]ncc2c1] |
| A-54 | 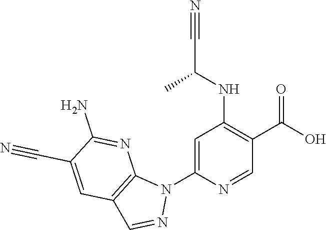 [SMILES: C[C@H](C#N)Nc1cc(-n2ncc3cc(C#N)c(N)nc32)ncc1C(=O)O] | 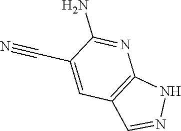 [SMILES: N#Cc1cc2cn[nH]c2nc1N] |
| A-61 | 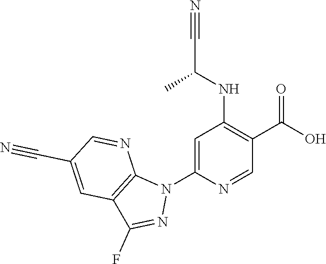 [SMILES: C[C@H](C#N)Nc1cc(-n2nc(F)c3cc(C#N)cnc32)ncc1C(=O)O] | 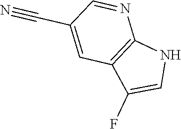 [SMILES: N#Cc1cnc2[nH]cc(F)c2c1] |
| A-68 | 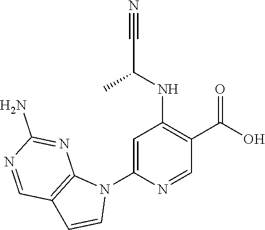 [SMILES: C[C@H](C#N)Nc1cc(-n2ccc3cnc(N)nc32)ncc1C(=O)O] | 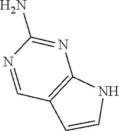 |
| A-74 | 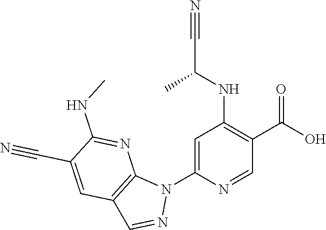 [SMILES: CNc1nc2c(cnn2-c2cc(N[C@H](C)C#N)c(C(=O)O)cn2)cc1C#N] | 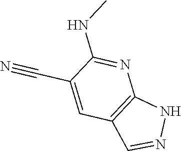 |
| A-76 | 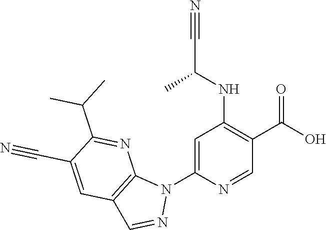 [SMILES: CC(C)c1nc2c(cnn2-c2cc(N[C@H](C)C#N)c(C(=O)O)cn2)cc1C#N] | 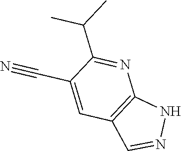 |
| A-29-I | 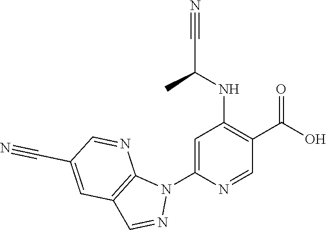 [SMILES: C[C@@H](C#N)Nc1cc(-n2ncc3cc(C#N)cnc32)ncc1C(=O)O] | 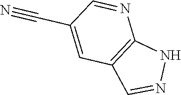 [SMILES: N#Cc1cnc2[nH]ncc2c1] |
| | *Synthesized with (2S)-2-aminopropanamide to afford the enantiomer | |
| A-60 | 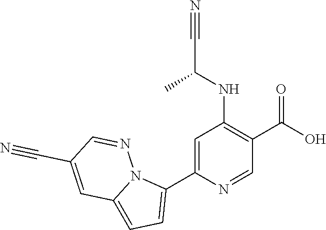 [SMILES: C[C@H](C#N)Nc1cc(-c2ccc3cc(C#N)cnn23)ncc1C(=O)O] | 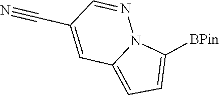 |
| | *C-C Suzuki coupling, typified in route to A-40, used to attach heterobicycle | |
| A-62 | 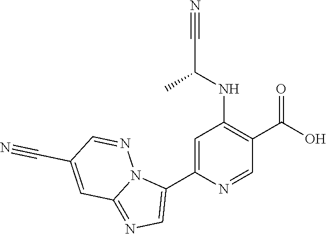 [SMILES: C[C@H](C#N)Nc1cc(-c2cnc3cc(C#N)cnn23)ncc1C(=O)O] | 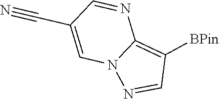 |
| | *C-C Stille coupling, typified in route to A-49, used to attach heterobicycle | |
| A-73 | 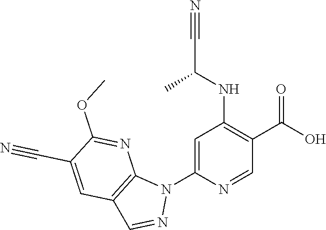 [SMILES: COc1nc2c(cnn2-c2cc(N[C@H](C)C#N)c(C(=O)O)cn2)cc1C#N] | 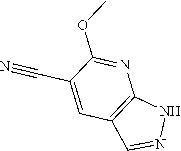 |
| A-75 | 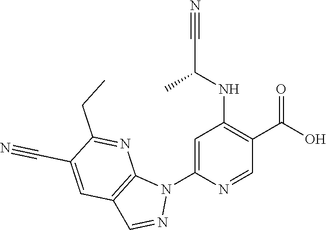 [SMILES: CCc1nc2c(cnn2-c2cc(N[C@H](C)C#N)c(C(=O)O)cn2)cc1C#N] | 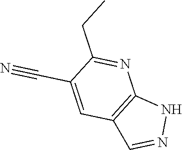 |
| A-79 | 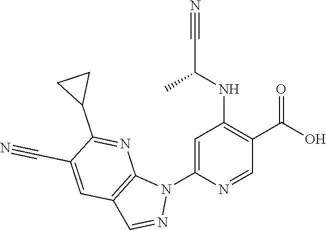 [SMILES: C[C@H](C#N)Nc1cc(-n2ncc3cc(C#N)c(C4CC4)nc32)ncc1C(=O)O] | 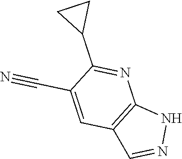 [SMILES: N#Cc1cc2cn[nH]c2nc1C1CC1] |

General Procedure TBM-7. The scheme shown below for the synthesis of A-48 is provided as a representative synthesis for General Procedure TBM-7.

### Example S30. 4-[(1-cyanocyclopropyl)amino]-6-(5-cyanopyrazolo[3,4-b]pyridin-2-yl)pyridine-3-carboxylic acid (A-48)

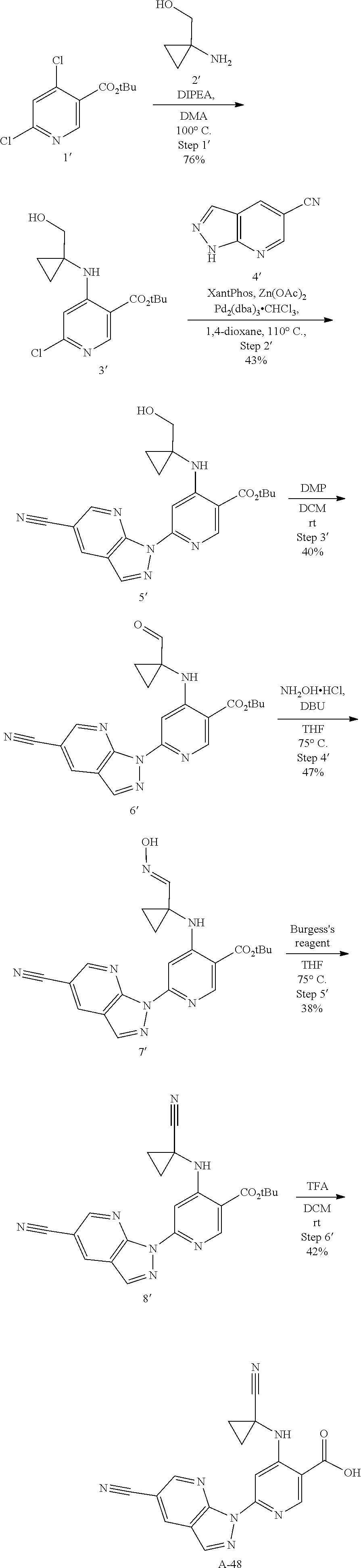

Step 1′. Preparation of tert-butyl 6-(5-cyanopyrazolo[3,4-b]pyridin-1-yl)-4-[[1-(hydroxymethyl)cyclopropyl]amino]pyridine-3-carboxylate (3′): An RBF was loaded with tert-butyl 4,6-dichloropyridine-3-carboxylate (1′) (1.2 g, 4.84 mmol), DMA (12.0 mL) and 2-amino-2-methyl-butan-1-ol (2′) (0.85 mL, 6.77 mmol) and heated at 100° C. for 16 h. After 16 h, DCM and water were added and the phases separated. The aqueous phase was extracted two more times with DCM. Combined organics were washed three times with water and then brine, dried over MgSO₄ and concentrated in vacuum to afford tert-butyl 6-chloro-4-[[1-(hydroxymethyl)cyclopropyl]amino]pyridine-3-carboxylate (3′) (1.2 g, 76% yield) as an off-white solid.

LCMS method 1: 89% purity at 215 nm, [M+H]⁺=299.2.

¹H NMR (400 MHz, DMSO-d6) δ ppm 0.70-0.79 (m, 2H), 0.88-0.95 (m, 2H), 1.53 (s, 9H), 3.44 (d, J=5.9 Hz, 2H), 4.95 (t, J=5.7 Hz, 1H), 6.99 (s, 1H), 8.38 (s, 1H), 8.49 (s, 1H).

Step 2′. Preparation of tert-butyl 6-(5-cyanopyrazolo[3,4-b]pyridin-1-yl)-4-[[1-(hydroxymethyl)cyclopropyl]amino]pyridine-3-carboxylate (5′): A flame-dried sealed tube was charged with tert-butyl 6-chloro-4-[[1-(hydroxymethyl)cyclopropyl]amino]pyridine-3-carboxylate (3′) (630 mg, 2.11 mmol), 1H-pyrazolo[3,4-b]pyridine-5-carbonitrile (4′) (303 mg, 2.11 mmol), Xantphos (244 mg, 0.42 mmol), Zn(OAc)₂ (386 mg, 2.11 mmol) and 1,4-Dioxane (10.0 mL). The mixture was degassed with nitrogen for 5 min, Pd₂(dba)₃·CHCl₃ (218 mg, 0.21 mmol) was added and degassing continued for 5 more min. After stirring for 16 h at 110° C., reaction mixture was filtered through Celite®, and filter cake was washed with DCM. The filtrate was concentrated and the residue was purified by flash column chromatography (DCM/MeOH) to afford tert-butyl 6-(5-cyanopyrazolo[3,4-b]pyridin-1-yl)-4-[[1-(hydroxymethyl)cyclopropyl]amino]pyridine-3-carboxylate (5′) (370 mg, 43% yield).

LCMS method 1: 99.9% purity at 215 nm, [M+H]⁺=407.2.

¹H NMR (400 MHz, DMSO-d6) δ ppm 0.81-0.87 (m, 2H), 0.92-0.99 (m, 2H), 1.57 (s, 9H), 3.52 (d, J=5.9 Hz, 2H), 4.95 (t, J=5.7 Hz, 1H), 7.79 (s, 1H), 8.45 (s, 1H), 8.67 (s, 1H), 8.76 (s, 1H), 9.03 (d, J=2.0 Hz, 1H), 9.07 (d, J=2.0 Hz, 1H).

Step 3′. Preparation of tert-butyl 6-(5-cyanopyrazolo[3,4-b]pyridin-1-yl)-4-[(1-formylcyclopropyl)amino]pyridine-3-carboxylate (6′): DMP (579 mg, 1.37 mmol) was added to a suspension of tert-butyl 6-(5-cyanopyrazolo[3,4-b]pyridin-1-yl)-4-[[1-(hydroxymethyl)cyclopropyl]amino]pyridine-3-carboxylate (5′) (370 mg, 0.91 mmol) in DCM (10.0 mL) at rt. After 6 h stirring, reaction mixture was diluted with 10% NaHCO₃ and MeTHF. The aqueous layer extracted with MeTHF 2×. The combined organics were dried by filtering through a pad of Na₂SO₄ and concentrated under reduced pressure. The residue was purified by flash column chromatography (DCM/MeOH) to give tert-butyl 6-(5-cyanopyrazolo[3,4-b]pyridin-1-yl)-4-[(1-formylcyclopropyl)amino]pyridine-3-carboxylate (6′) (150 mg, 0.3705 mmol, 40% yield).

LCMS method 1: 65.0% purity at 215 nm, [M+H]⁺=405.2

¹H NMR (400 MHz, DMSO-d6) δ ppm 1.53-1.56 (m, 2H), 1.59 (s, 9H), 1.76-1.83 (m, 2H), 7.50 (s, 1H), 8.51 (s, 1H), 8.66 (s, 1H), 8.81 (s, 1H), 8.93 (s, 1H), 9.02 (d, J=2.0 Hz, 1H), 9.06 (d, J=2.0 Hz, 1H).

Step 4′. Preparation of tert-butyl 6-(5-cyanopyrazolo[3,4-b]pyridin-1-yl)-4-[[1H-(hydroxyiminomethyl)cyclopropyl]amino]pyridine-3-carboxylate (7′): To a suspension of tert-butyl 6-(5-cyanopyrazolo[3,4-b]pyridin-1-yl)-4-[(1-formylcyclopropyl)amino]pyridine-3-carboxylate (6′) (260 mg, 0.64 mmol) in DCM (6.0 mL) was added Hydroxylamine hydrochloride (49 mg, 0.71 mmol) followed by DBU (0.29 mL, 1.93 mmol) at rt. After 48 h stirring, the reaction mixture was diluted with 10% NaHCO₃ and MeTHF. The aq layer extracted to MeTHF 2×. The combined organics were dried by filtering through a pad of Na₂SO₄ and concentrated under reduced pressure. The crude tert-butyl 6-(5-cyanopyrazolo[3,4-b]pyridin-1-yl)-4-[[1-(hydroxyiminomethyl)cyclopropyl]amino]pyridine-3-carboxylate (7′) (210 mg, 47% yield) was used in the next step without further purification.

LCMS method 1: 61% purity at 215 nm, [M+H]⁺=420.2

Step 5′. Preparation of tert-butyl 4-[(1-cyanocyclopropyl)amino]-6-(5-cyanopyrazolo[3,4-b]pyridin-1-yl)pyridine-3-carboxylate (8′): Burgess reagent (0.65 mL, 2.0 mmol) was added to a suspension of tert-butyl 6-(5-cyanopyrazolo[3,4-b]pyridin-1-yl)-4-[[1-hydroxyiminomethylcyclopropyl]amino]pyridine-3-carboxylate (7′) (210 mg, 0.5 mmol) in THF (5.0 mL) at RT in a sealed tube. The tube was sealed and the reaction mixture was heated to 75° C. The progress of reaction was followed by HPLC. The reaction mixture was concentrated under reduced pressure. The residue was purified by flash column chromatography (DCM/MeOH) to afford tert-butyl 4-[(1-cyanocyclopropyl)amino]-6-(5-cyanopyrazolo[3,4-b]pyridin-1-yl)pyridine-3-carboxylate (8′) (120 mg, 38% yield) as an off-white solid.

LCMS method 1: 64% purity at 215 nm, [M+H]⁺=402.2.

¹H NMR (400 MHz, DMSO-d6) δ ppm 1.09-1.11 (m, 2H), 1.12 (s, 2H), 1.59 (s, 9H), 8.00 (s, 1H), 8.66 (s, 1H), 8.74 (s, 1H), 8.86 (s, 1H), 9.06 (d, J=2.0 Hz, 1H), 9.12 (d, J=2.0 Hz, 1H).

Step 6′. Preparation of 4-[(1-cyanocyclopropyl)amino]-6-(5-cyanopyrazolo[3,4-b]pyridin-2-yl)pyridine-3-carboxylic acid (A-48): To a solution of tert-butyl 4-[(1-cyanocyclopropyl)amino]-6-(5-cyanopyrazolo[3,4-b]pyridin-2-yl)pyridine-3-carboxylate (8′) (120 mg, 0.15 mmol) in anhydrous DCM (4.0 mL) was added TFA (2.0 mL, 8.0 mmol) at room temp. After 24 h stirring, reaction mixture was evaporated to dryness and the residue triturated in EtOAc to yield a white solid which was purified by reversed phase C18 column chromatography (MeOH/HCO₂NH₄ in water) to afford 4-[(1-cyanocyclopropyl)amino]-6-(5-cyanopyrazolo[3,4-b]pyridin-2-yl)pyridine-3-carboxylic acid (A-48) (22 mg, 42% yield).

LCMS method 1: 99.9% purity at 215 nm, [M+H]⁺=346.2.

¹H NMR (400 MHz, DMSO-d6) δ ppm 1.39-1.45 (m, 2H), 1.69-1.75 (m, 2H), 7.80 (br. s, 1H), 8.70 (s, 1H), 8.80 (s, 1H), 9.04 (d, J=1.7 Hz, 1H), 9.09 (d, J=1.2 Hz, 1H).

TBM intermediates prepared using General Procedure TBM-7 are summarized in Table 20.

TABLE 20
TBM Intermediates Prepared via General Procedure TBM-7

| | Structure | Amine |
|---|---|---|
| A-44 | 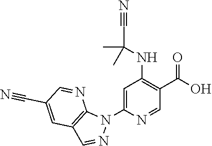 [SMILES: CC(C)(C#N)Nc1cc(-n2ncc3cc(C#N)cnc32)ncc1C(=O)O] | 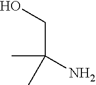 |
| A-48 | 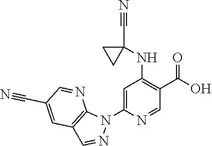 [SMILES: N#Cc1cnc2c(cnn2-c2cc(NC3(C#N)CC3)c(C(=O)O)cn2)c1] | 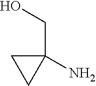 |

General Procedure TBM-8. The scheme shown below for the synthesis of A-55 is provided as a representative synthesis for General Procedure TBM-8.

### Example S30. 4-[(4-carbamoylcyclohexyl)amino]-6-(5-cyanopyrazolo[3,4-b]pyridin-1-yl)pyridine-3-carboxylic acid; trifluoroacetic acid (A-55)

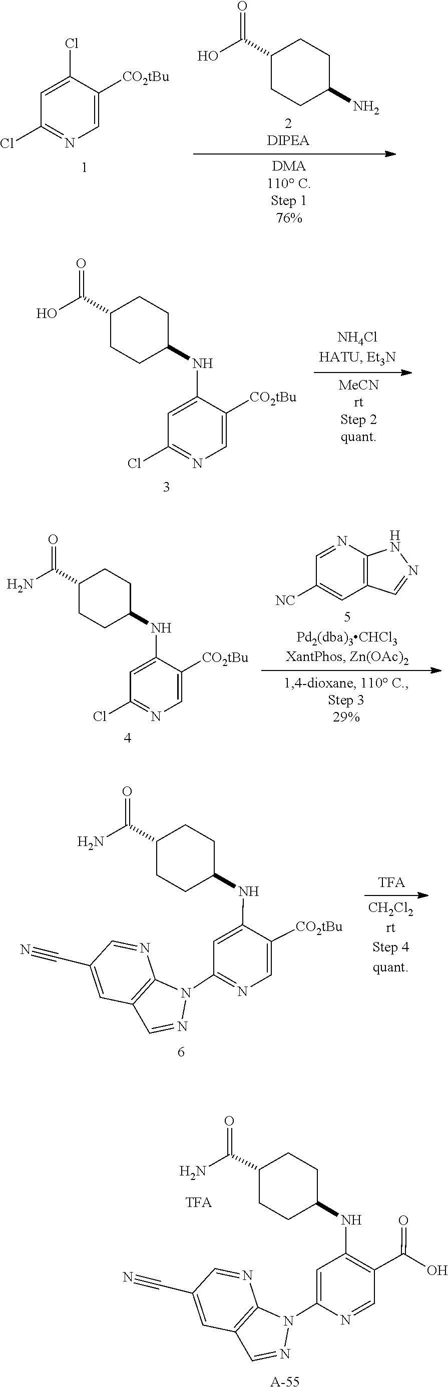

Step 1. Preparation of 4-[(5-tert-butoxycarbonyl-2-chloro-4-pyridyl)amino]cyclohexanecarboxylic acid (3): To a solution of tert-butyl 4,6-dichloropyridine-3-carboxylate 1 (250 mg, 1.01 mmol, 1.0 eq.) and 4-aminocyclohexanecarboxylic acid 2 (288 mg, 2.02 mmol, 2.0 eq.) in DMA (4.0 mL), was added DIPEA (1.05 mL, 6.05 mmol, 6.0 eq.). The reaction mixture was stirred at 110° C. After 2 days, LCMS showed full conversion. The reaction mixture was directly purified by reverse phase flash chromatography (MeOH/0.1% HCOOH 5:95, then 5:95 to 100:0) to give 3 (272 mg, 74% yield) as a white solid.

LCMS method 1: 97.4% purity at 215 nm, [M+H]⁺=355.0.

¹H NMR (400 MHz, DMSO-d₆) δ ppm 1.21-1.35 (m, 2H), 1.44-1.61 (m, 11H), 1.84-2.03 (m, 4H), 2.17-2.29 (m, 1H), 3.47-3.62 (m, 1H), 6.91 (s, 1H), 7.99 (d, J=8.1 Hz, 1H), 8.47 (s, 1H), 12.09 (s, 1H).

Step 2. Preparation of tert-butyl 4-[(4-carbamoylcyclohexyl)amino]-6-chloro-pyridine-3-carboxylate (4): A mixture of 4-[(5-tert-butoxycarbonyl-2-chloro-4-pyridyl)amino]cyclohexanecarboxylic acid 3 (275 mg, 0.78 mmol, 1.0 eq.), NH₄C₁ (62.2 mg, 1.16 mmol, 1.5 eq.), HATU (295 mg, 0.78 mmol, 1.0 eq.), Et₃N (0.4 mL, 2.33 mmol, 3.0 eq.) in MeCN (10.1 mL) was stirred at room temperature. After 1 h, LCMS showed full conversion. The reaction was then concentrated and the residue was purified by reverse phase flash chromatography (MeOH/0.1% HCOOH 5:95, then 5:95 to 100:0) to give 4 (290 mg, quantitative yield) as a white solid.

LCMS method 1: 97.0% purity at 215 nm, [M+H]⁺=354.0.

¹H NMR (400 MHz, DMSO-d6) δ ppm 1.17-1.30 (m, 2H), 1.45-1.61 (m, 11H), 1.73-1.83 (m, 2H), 1.94-2.03 (m, 2H), 2.04-2.15 (m, 1H), 3.45-3.58 (m, 1H), 6.68 (br s, 1H), 6.90 (s, 1H), 7.23 (br s, 1H), 7.97 (d, J=8.1 Hz, 1H), 8.47 (s, 1H).

Step 3. Preparation of tert-butyl 4-[(4-carbamoylcyclohexyl)amino]-6-(5-cyanopyrazolo[3,4-b]pyridin-1-yl)pyridine-3-carboxylate (6): tert-Butyl 4-[(4-carbamoylcyclohexyl)amino]-6-chloro-pyridine-3-carboxylate 4 (290 mg, 0.82 mmol, 1.0 eq.), Xantphos (94.8 mg, 0.16 mmol, 0.2 eq.), 1H-pyrazolo[3,4-b]pyridine-5-carbonitrile 5 (149 mg, mmol, 1.2 eq.) and Zn(OAc)₂ (150 mg, 0.82 mmol, 1.0 eq.) were added to 1,4-dioxane (4.1 mL) and nitrogen was sparged through the mixture. After 10 min, Pd₂(dba)₃·CHCl₃ (75.0 mg, mmol, 0.1 eq.) was added and sparging was continued. After 10 min, the tube was sealed and the reaction mixture was stirred at 110° C. After 16 h, LCMS showed full conversion. The reaction mixture was filtered through celite, and the cake was washed with CH₂Cl₂. The filtrate was concentrated and the residue was purified by normal phase flash chromatography (MeOH/CH₂Cl_{2 0:100} to 10:90 over 20 CV) to give 6 (110 mg, 23% yield) as an orange solid.

LCMS method 1: 80.7% purity at 215 nm, [M+H]⁺=462.2.

¹H NMR (400 MHz, DMSO-d6) δ ppm 1.26-1.40 (m, 2H), 1.44-1.67 (m, 11H), 1.79-1.89 (m, 2H), 2.09-2.20 (m, 3H), 3.42-3.54 (m, 1H), 6.73 (br s, 1H), 7.26 (br s, 1H), 7.50 (s, 1H), 8.06 (br d, J=7.3 Hz, 1H), 8.66 (s, 1H), 8.75 (s, 1H), 9.02 (d, J=2.0 Hz, 1H), 9.07 (d, J=2.0 Hz, 1H).

Step 4. Preparation of 4-[(4-carbamoylcyclohexyl)amino]-6-(5-cyanopyrazolo[3,4-b]pyridin-1-yl)pyridine-3-carboxylic acid; trifluoroacetic acid (A-55): To a solution of tert-butyl 4-[(4-carbamoylcyclohexyl)amino]-6-(5-cyanopyrazolo[3,4-b]pyridin-1-yl)pyridine-3-carboxylate 6 (100 mg, 0.22 mmol, 1.0 eq.) in CH₂Cl₂ (4.8 mL) was added TFA (0.41 mL, 5.42 mmol, 25 eq.). The reaction mixture was stirred at room temperature. After 1 h, LCMS showed full conversion. The mixture was concentrated to dryness and residual TFA was chased with toluene (2×) and MeCN (2×) to give A-55 (120 mg, quantitative yield) as a trifluoroacetic acid salt.

LCMS method 1: 95.7% purity at 215 nm, [M-CF₃COOH+H]⁺=406.2.

¹H NMR (400 MHz, DMSO-d6) δ ppm 1.30-1.43 (m, 2H), 1.52-1.63 (m, 2H), 1.83-1.92 (m, 2H), 2.11-2.22 (m, 3H), 3.48-3.61 (m, 1H), 6.75 (br s, 1H), 7.27 (br s, 1H), 7.69 (br s, 1H), 8.60 (br d, J=7.3 Hz, 1H), 8.75 (br d, J=7.3 Hz, 2H), 9.07 (d, J=1.7 Hz, 1H), 9.15 (d, J=1.7 Hz, 1H).

TBM intermediates prepared using General Procedure TBM-8 are summarized in Table 21.

TABLE 21
TBM Intermediates Prepared via General Procedure TBM-8

| | Structure |
|---|---|
| A-55 | 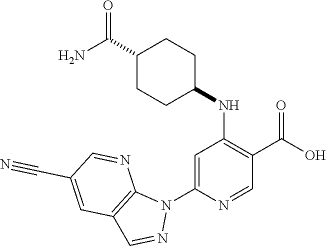 |

General Procedure TBM-9. The following scheme shows the synthetic route for General Procedure TBM-9.

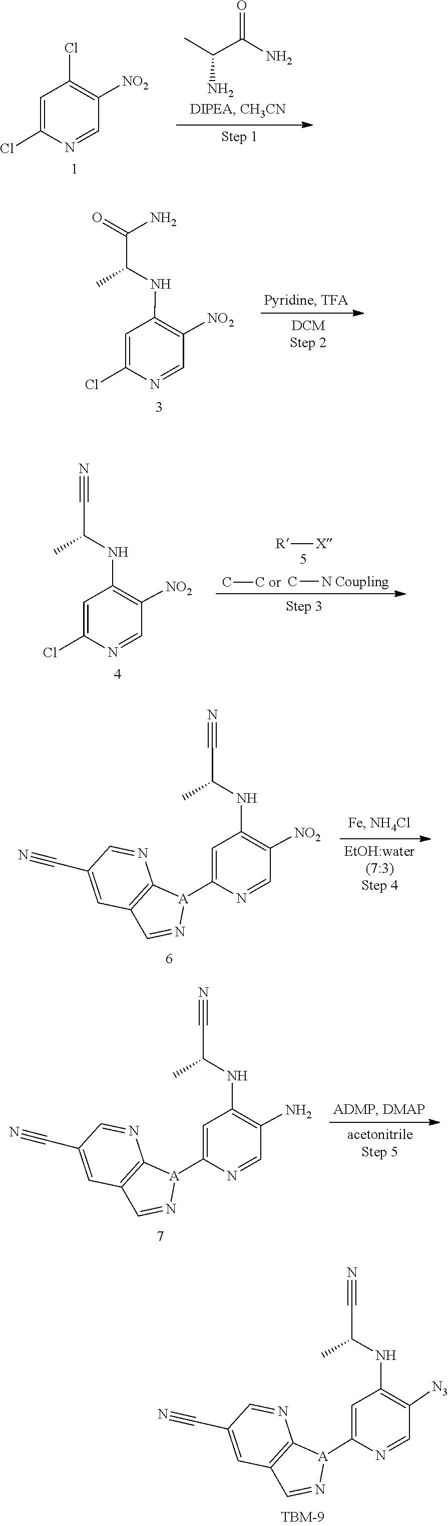

wherein R′ is a bicyclic heteroaryl, X″ is a chemical moiety capable of undergoing Pd-catalyzed coupling, and A is C or N.

Step 1. Preparation of (R)-2-((2-chloro-5-nitropyridin-4-yl)amino)propanamide 3: To a stirred solution of 2,4-dichloro-5-nitropyridine 1 (1.0 eq.) in acetonitrile (0.5 M) was added (R)-2-aminopropanamide hydrochloride 2 (1.2 eq.) followed by DIPEA (3.0 eq.) under Na atmosphere at RT. Then, the reaction was heated to 40° C. The reaction was cooled down to RT, poured into ice cold water and extracted with ethyl acetate. The combined organic layer was washed with brine (2×250 mL), dried over sodium sulphate and concentrated under reduced pressure to afford product 3.

Step 2. Preparation of (R)-2-((2-chloro-5-nitropyridin-4-yl)amino)propanenitrile 4: To a stirred solution of (R)-2-((2-chloro-5-nitropyridin-4-yl)amino)propanamide 3 (1.0 eq.) in DCM (0.3 M) at 0° C. was added pyridine (10.0 eq.) followed by trifluoroacetic anhydride (5.0 eq.) over a period of 10 min. Then, the reaction mixture was slowly warmed to room temperature. The reaction mixture was slowly quenched with ice cold water and extracted with DCM. The combined organic layer was washed with 1.5 N HCl solution and brine, dried over sodium sulphate and concentrated under reduced pressure to afford compound 4.

Step 3. Preparation of cross coupling compound 6: To a stirred solution of (R)-2-((2-chloro-5-nitropyridin-4-yl)amino)propanenitrile 4 (1.0 eq.) and coupling partner R-X 5 (1.0 eq.) in 1,4-dioxane (0.1 M) was added zinc acetate (0.6 eq.), K₂CO₃ (2.0 eq.) under Na atmosphere and the purging was continued for 10 min. Then, Xantphos (0.1 eq.) and Pd₂(dba)₃ (0.05 eq.) was added under Na atmosphere and the purging was continued for another 5 min. Then, the reaction mixture was stirred at 100° C. The reaction was cooled to RT and filtered through a celite bed, and the residue (filter cake) was washed with DCM. The filtrate was concentrated under reduced pressure to give the crude product. The crude product was purified by column chromatography using silica gel with ethyl acetate in pet ether to give compound 6.

Step 4. Preparation of reduced compound 7: To a stirred solution of 6 (1.0 eq.) in ethanol:water (7:3) at RT, iron (5.0 eq.) and ammonium chloride (2.5 eq.) were added and stirred at 85° C. The reaction mixture was cooled to RT and filtered through celite bed, the bed was washed with DCM and concentrated under reduced pressure to give crude product. The crude product was dissolved in water and stirred for 10 min at RT. Then, the crude product was filtered through Buchner funnel, washed with water, and dried to afford amine 7 that was used in the next step without further purification.

Step 5. Preparation of azide compound TBM-9: To a stirred solution of 7 (1.0 eq.) in acetonitrile (0.2 M), ADMP (2.5 eq.) and DMAP (2.5 eq.) were added at room temperature and stirred at RT under nitrogen atmosphere. The reaction mixture was treated with ice-cold water and extracted with ethyl acetate. The combined organic layer was dried over sodium sulphate, and then concentrated under reduced pressure to give azide TBM-9 and was used in the next step without further purification.

### Example S31. (R)-1-(5-azido-4-((1-cyanoethyl)amino)pyridin-2-yl)-1H-pyrazolo[3,4-b]pyridine-5-carbonitrile (A-57)

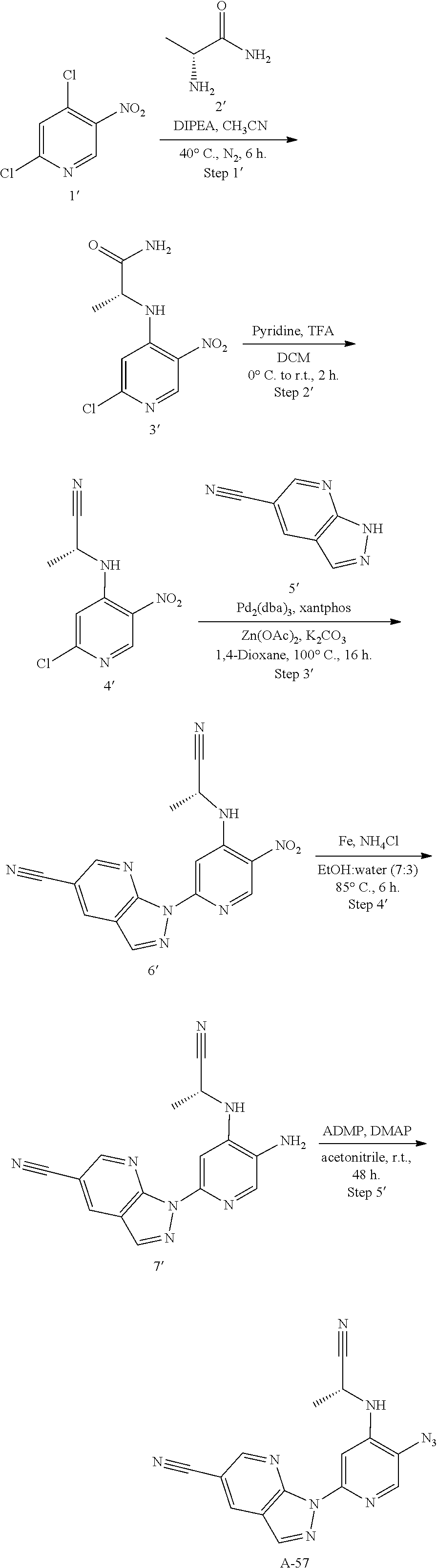

LCMS Method 1. Column: Kinetex XB-C18 75×3.0 mm, 2.6 Temperature: 45° C., Flow: 1.0 mL/min, run time: 5.0 min. Mobile Phase Conditions: Mobile Phase-A: 5.0 mm Ammonium formate pH 3.3:CH₃CN (98:02), Mobile Phase-B: CH₃CN: Buffer (98:02), Gradient: Initial 80% Mobile Phase A and 20% Mobile Phase B linear gradient to 100% Mobile Phase B for 4.0 min then hold for 0.6 minute. MSD positive.

UPLC Method 2. Column: Zorbax SB-C18 50×2.1 mm, 1.8 Temperature: 45° C., Flow: 0.7 mL/min, run time: 3.0 min. Mobile Phase Conditions: Mobile Phase-A: 5.0 mM Ammonium acetate:CH₃CN (95:05), Mobile Phase-B: CH₃CN: 5.0 mM Ammonium acetate (95:05), Gradient: Initial 80% Mobile Phase A and 20% Mobile Phase B linear gradient to 100% Mobile Phase B for 2.0 min then hold for 0.3 minute. MSD positive.

Step 1′. Preparation of (R)-2-((2-chloro-5-nitropyridin-4-yl)amino)propanamide 3′: To a stirred solution of 2,4-dichloro-5-nitropyridine 1′ (20.0 g, 104 mmol, 1.0 eq.) in acetonitrile (200 mL) was added (R)-2-aminopropanamide hydrochloride 2′ (15.49 g, 124 mmol, 1.2 eq.) followed by DIPEA (54.3 mL, 311 mmol, 3.0 eq.) under N₂ atmosphere at RT. Then, the reaction was heated to 40° C. for 6 h. The reaction was cooled down to RT, poured into ice cold water (500 mL) and extracted with ethyl acetate (3×700 mL). The combined organic layer was washed with brine (2×250 mL), dried over sodium sulphate and concentrate under reduced pressure to afford (R)-2-((2-chloro-5-nitropyridin-4-yl)amino)propanamide 3′ (27 g, 97% yield) as a pale yellow solid.

LCMS method 1: retention time: 0.876 min, 91.45% purity at 220 nm, [M+H]⁺=245.0.

¹H NMR (300 MHz, DMSO-d6): δ ppm 1.42 (d, J=8.8 Hz, 3H), 4.34-4.39 (m, 1H), 6.90 (s, 1H), 7.46 (s, 1H), 7.69 (s, 1H), 8.66-8.68 (m, 1H), 8.93 (s, 1H).

Step 2′. Preparation of (R)-2-((2-chloro-5-nitropyridin-4-yl)amino)propanenitrile 4′: To a stirred solution of (R)-2-((2-chloro-5-nitropyridin-4-yl)amino)propanamide 3′ (27.0 g, 100 mmol, 1.0 eq.) in DCM (270 mL) at 0° C. was added pyridine (81 mL, 1004 mmol, 10.0 eq.) followed by trifluoroacetic anhydride (70.9 mL, 502 mmol, 5.0 eq.) over a period of 10 min. Then, the reaction mixture was slowly warmed to room temperature and the stirring was continued for 2 h. The reaction mixture was slowly quenched with ice cold water (300 mL) and extracted with DCM (2×300 mL). The combined organic layer was washed with 1.5 N HCl solution (2×200 mL) and brine (2×250 mL), dried over sodium sulphate and concentrated under reduced pressure to afford (R)-2-((2-chloro-5-nitropyridin-4-yl)amino)propanenitrile 4′ (21 g, 89% yield) as a pale yellow solid.

LCMS method 1: retention time: 1.583 min, 96.3% purity at 220 nm, [M+H]⁺=227.0.

¹H NMR (400 MHz, DMSO-d6): δ ppm 1.68 (d, J=7.2 Hz, 3H), 5.18-5.22 (m, 1H), 7.35 (s, 1H), 8.42 (d, J=8.4 Hz, 1H), 8.95 (s, 1H).

Step 3′. Preparation of (R)-1-(4-((1-cyanoethyl)amino)-5-nitropyridin-2-yl)-1H-pyrazolo[3,4-b]pyridine-5-carbonitrile 6′: To a stirred solution of (R)-2-((2-chloro-5-nitropyridin-4-yl)amino)propanenitrile 4′ (1.965 g, 8.33 mmol, 1.0 eq.) and 1H-pyrazolo[3,4-b]pyridine-5-carbonitrile 5′ (1.2 g, 8.33 mmol, 1.0 eq.) in 1,4-dioxane (70 mL) was added zinc acetate (0.917 g, 5.00 mmol, 0.6 eq.), K₂CO₃ (2.301 g, 16.65 mmol, 2.0 eq.) under N₂ atmosphere and the purging was continued for 10 min. Then, Xantphos (0.482 g, 0.833 mmol, eq.) and Pd₂(dba)₃ (0.381 g, 0.416 mmol, 0.05 eq.) was added under N₂ atmosphere and the purging was continued for another 5 min. Then, the reaction mixture was stirred at 100° C. for 16 h. The reaction was cooled to RT and filtered through a celite bed, and the residue (filter cake) was washed with DCM (500 mL). The filtrate was concentrated under reduced pressure to give the crude product. The crude product was purified by column chromatography using silica gel (230-400 mesh) with 80% ethyl acetate in pet ether to give (R)-1-(4-((1-cyanoethyl)amino)-5-nitropyridin-2-yl)-1H-pyrazolo[3,4-b]pyridine-5-carbonitrile 6′ (1.5 g, 53.9% yield) as a yellow solid.

¹H NMR (400 MHz, DMSO-d6): δ ppm 1.77 (d, J=7.2 Hz, 3H), 5.21-5.25 (m, 1H), 7.86 (s, 1H), 8.53 (d, J=7.6 Hz, 1H), 8.78 (s, 1H), 9.03-9.11 (m, 2H), 9.22 (s, 1H).

Step 4′. Preparation of (R)-1-(5-amino-4-((1-cyanoethyl)amino)pyridin-2-yl)-1H-pyrazolo[3,4-b]pyridine-5-carbonitrile 7′: To a stirred solution of (R)-1-(4-((1-cyanoethyl)amino)-5-nitropyridin-2-yl)-1H-pyrazolo[3,4-b]pyridine-5-carbonitrile 6′ (1.5 g, 4.49 mmol) in 100 mL of ethanol:water (7:3) at RT, iron (1.253 g, 22.44 mmol) and ammonium chloride (0.600 g, 11.22 mmol) were added and stirred for 6 h at 85° C. The reaction mixture was cooled to RT and filtered through celite bed, the bed was washed with DCM (500 mL) and concentrated under reduced pressure to give crude product. The crude product was dissolved in water and stirred for 10 min at RT. Then, the crude product was filtered through Buchner funnel, washed with water, and dried to afford (R)-1-(5-amino-4-((1-cyanoethyl)amino)pyridin-2-yl)-1H-pyrazolo[3,4-b]pyridine-5-carbonitrile 7′ (1.2 g, 61.5% yield) as brown solid, which was used in the next step without further purification.

UPLC method 2: retention time: 0.561 min, [M+H]⁺=305.0.

Step 5′. Preparation of (R)-1-(5-azido-4-((1-cyanoethyl)amino)pyridin-2-yl)-1H-pyrazolo[3,4-b]pyridine-5-carbonitrile A-57: To a stirred solution of (R)-1-(5-amino-4-((1-cyanoethyl)amino)pyridin-2-yl)-1H-pyrazolo[3,4-b]pyridine-5-carbonitrile 7′ (1.2 g, 3.94 mmol, 1.0 eq.) in acetonitrile (20 mL), ADMP (2.81 g, 9.86 mmol, 2.5 eq.) and DMAP (1.204 g, 9.86 mmol, 2.5 eq.) were added at room temperature and stirred for 16 h at RT under nitrogen atmosphere. The reaction mixture was treated with ice-cold water (100 mL) and extracted with ethyl acetate (3×150 mL). The combined organic layer was dried over sodium sulphate, and then concentrated under reduced pressure to give (R)-1-(5-azido-4-((1-cyanoethyl)amino)pyridin-2-yl)-1H-pyrazolo[3,4-b]pyridine-5-carbonitrile A-57 (1.3 g, 56.8% yield) as a gummy liquid, which was used in the next step without further purification. LCMS method 1: retention time: 1.484 min, [M+H]⁺=331.0.

### Example S32. (R)-7-(5-azido-4-((1-cyanoethyl)amino)pyridin-2-yl)pyrrolo[1,2-b]pyridazine-3-carbonitrile (A-87)

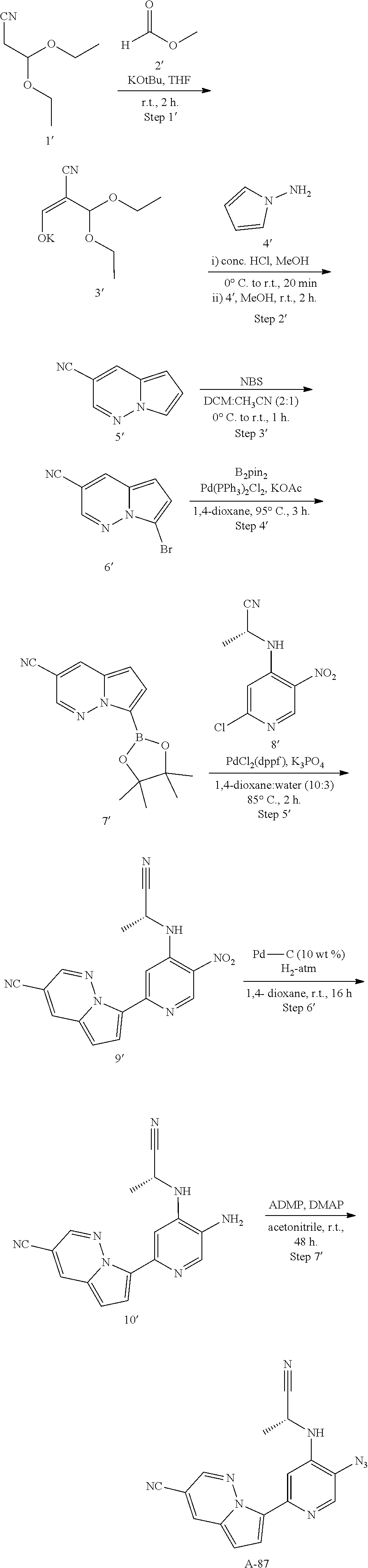

UPLC Method 1. Column: Aquity BEH-C18, 50×3.0 mm, 1.7 Temperature: RT, Flow: 0.7 mL/min, run time: 2.6 min. Mobile Phase Conditions: Mobile Phase-A: 5.0 mm Ammonium formate pH 3.3:CH₃CN (98:02), Mobile Phase-B: CH₃CN: Buffer (98:02), Gradient: Initial 80% Mobile Phase A and 20% Mobile Phase B linear gradient to 98% Mobile Phase B for 1.5 min then hold for 0.5 minute. MSD positive

UPLC Method 2. Column: Aquity BEH-C18, 50×2.1 mm, 1.7 Temperature: RT, Flow: 0.7 mL/min, run time: 2.5 min. Mobile Phase Conditions: Mobile Phase-A: 0.1% TFA in Water, Mobile Phase-B: 0.1% TFA in CH₃CN, Gradient: Initial 80% Mobile Phase A and 20% Mobile Phase B linear gradient to 98% Mobile Phase B for 1.5 min then hold for 0.5 minute. MSD positive

Step 1′. Preparation of potassium (Z)-2-cyano-3,3-diethoxyprop-1-en-1-olate 3′: To a stirred solution of 3,3-diethoxypropanenitrile 1′ (10 g, 69.8 mmol, 1.0 eq.) and methyl formate 2′ (5.14 mL, 84 mmol, 1.2 eq.) in THF (20 mL) was added KOtBu 1.0M in THF (77 mL, 77 mmol, 1.1 eq.) over a period of 45 min while the internal temperature was maintained at ° C. The reaction mixture was stirred for 2 h at RT after addition. Then, petroleum ether (170 mL) was added, and the stirring was continued further for another 20 min. The slurry was filtered, and the residue (filter cake) was washed with petroleum ether/THF (1:1) and dried to afford potassium 3′ (13.5 g, 92% yield) as an off-white solid.

¹H NMR (400 MHz, DMSO-d6): δ ppm 1.03-1.09 (m, 6H), 3.31-3.38 (m, 2H), 3.4-3.47 (m, 2H), 5.15 and 5.22 (s, 1H).

Step 2′. Preparation of pyrrolo[1,2-b]pyridazine-3-carbonitrile 5′: To a stirred solution of potassium (Z)-2-cyano-3,3-diethoxyprop-1-en-1-olate 3′ (11.6 g, 55.4 mmol, 1.0 eq.) in MeOH (90 mL) at 0° C., was slowly added concentrated HCl (13.6 mL, 448 mmol, 3.0 eq.) and after the addition the mixture was stirred at RT for 20 min. Then, a solution of 1H-pyrrol-1-amine 4′ (2.275 g, 27.7 mmol, 0.5 eq.) in MeOH (20 mL) was added and the reaction mixture was stirred at RT for another 2 h. The reaction mixture was neutralized by the addition of saturated aqueous NaHCO₃ solution and then filtered through Buchner funnel. The residue was washed with cold water and dried to give pyrrolo[1,2-b]pyridazine-3-carbonitrile 5′ (6.2 g, 78% yield) as a yellow solid.

¹H NMR (400 MHz, DMSO-d6): δ ppm 6.95 (dd, J=1.2 Hz and 4.4 Hz, 1H), 7.11 (dd, J=2.8 Hz and 4.4 Hz, 1H), 8.18-8.19 (m, 1H), 8.46 (d, J=2.0 Hz, 1H), 8.72 (d, J=2.0 Hz, 1H).

Step 3′. Preparation of 7-bromopyrrolo[1,2-b]pyridazine-3-carbonitrile 6′: To a stirred solution of pyrrolo[1,2-b]pyridazine-3-carbonitrile 5′ (6.2 g, 43.3 mmol, 1.0 eq.) in DCM (150 mL) and acetonitrile (75 mL) at 0° C. was added NBS (6.94 g, 39.0 mmol, 0.9 eq.) and the reaction was stirred at RT for 1 h. After 1 h, the reaction was quenched by the addition of saturated aqueous NaHCO₃ solution. The mixture was extracted with DCM (2×200 mL) and the combined organic phases were dried over Na₂SO₄, filtered, and concentrated under reduced pressure. The crude product was purified by column chromatography using silica gel (230-400 mesh) with 30% ethyl acetate in pet ether to give 7-bromopyrrolo[1,2-b]pyridazine-3-carbonitrile 6′ (5.0 g, 52.0% yield) as a yellow solid.

¹H NMR (400 MHz, DMSO-d₆): δ ppm 7.10 (d, J=4.8 Hz, 1H), 7.32 (d, J=4.8 Hz, 1H), 8.65 (d, J=2.0 Hz, 1H), 8.76 (d, J=2.0 Hz, 1H).

Step 4′. Preparation of 7-(4,4,5,5-tetramethyl-1,3,2-dioxaborolan-2-yl)pyrrolo[1,2-b]pyridazine-3-carbonitrile 7′: To a stirred solution of 7-bromopyrrolo[1,2-b]pyridazine-3-carbonitrile 6′ (3.5 g, 15.76 mmol, 1.0 eq.) in 1,4-dioxane (50 mL) was added Bis(pinacolato)diboron (12.01 g, 47.3 mmol, 3.0 eq.) and KOAc (4.64 g, 47.3 mmol, 3.0 eq.). The solution was degassed with N₂ for 5 min, then Pd(PPh₃)₂Cl₂ (1.106 g, 1.576 mmol, 0.1 eq.) was added and degassing continued for another 5 min. Then, the reaction was heated at 95° C. for 3 h. The mixture was filtered through celite bed, and the residue (filter cake) was further washed with ethyl acetate (200 mL). The filtrate was concentrated under reduced pressure to give crude 7-(4,4,5,5-tetramethyl-1,3,2-dioxaborolan-2-yl)pyrrolo[1,2-b]pyridazine-3-carbonitrile 7′ (3.5 g) as a black solid, which was used in the next step without further purification.

Step 5′. Preparation of (R)-7-(4-((1-cyanoethyl)amino)-5-nitropyridin-2-yl)pyrrolo[1,2-b]pyridazine-3-carbonitrile 9′: To a stirred solution of (R)-2-((2-chloro-5-nitropyridin-4-yl)amino)propanenitrile 8′ (2.456 g, 10.84 mmol, 1.0 eq.) in 1,4-dioxane (20 mL) and H₂O (6 mL) was added K₃PO₄ (7.55 g, 43.4 mmol, 4.0 eq.) followed by 7-(4,4,5,5-tetramethyl-1,3,2-dioxaborolan-2-yl)pyrrolo[1,2-b]pyridazine-3-carbonitrile 7′ (3.5 g, 13.01 mmol, 1.2 eq.). The solution was degassed with Na for 5 min and then PdCl₂(dppf) (0.793 g, 1.084 mmol, 0.1 eq.) was added, and the reaction mixture was heated at 85° C. for 2 h. After 2 h, the reaction mixture was filtered through a celite bed and the celite bed was washed with ethyl acetate (500 mL). The combined organic layer was further washed with brine (200 mL), dried over sodium sulphate, and concentrate under reduced pressure to give the crude product (black oil). The crude product was purified by column chromatography using silica gel (230-400 mesh) with 40% ethyl acetate in pet ether to give (R)-7-(4-((1-cyanoethyl)amino)-5-nitropyridin-2-yl)pyrrolo[1,2-b]pyridazine-3-carbonitrile 9′ (1.8 g, 16.44% yield) as yellow solid.

UPLC method 1: retention time: 1.312 min, [M+H]⁺=334.0

Step 6′. Preparation of (R)-7-(5-amino-4-((l-cyanoethyl)amino)pyridin-2-yl)pyrrolo[1,2-b]pyridazine-3-carbonitrile 10′: To a stirred solution of (R)-7-(4-((1-cyanoethyl)amino)-5-nitropyridin-2-yl)pyrrolo[1,2-b]pyridazine-3-carbonitrile 9′ (1.8 g, 5.40 mmol, 1.0 eq.) in 1,4-dioxane (20 mL) was added 10% Pd—C(500 mg, 4.70 mmol, 0.87 eq.) and the reaction was stirred under hydrogen atmosphere for 16 h. Then, the reaction mixture was filtered through celite bed, the celite bed was washed with 1,4-dioxane (300 mL) and concentrated under reduced pressure to give the crude (R)-7-(5-amino-4-((1-cyanoethyl)amino)pyridin-2-yl)pyrrolo[1,2-b]pyridazine-3-carbonitrile 10′ (1.0 g) as brown solid, which was used in the next step without further purification.

UPLC method 2: retention time: 0.778 min, [M+H]⁺=304.0

Step 7′. Preparation of (R)-7-(5-azido-4-((1-cyanoethyl)amino)pyridin-2-yl)pyrrolo[1,2-b]pyridazine-3-carbonitrile A-87: To a stirred solution of (R)-7-(5-amino-4-((1-cyanoethyl)amino)pyridin-2-yl)pyrrolo[1,2-b]pyridazine-3-carbonitrile 10′ (1.0 g, 3.30 mmol, 1.0 eq.) in acetonitrile (10 mL), ADMP (2.350 g, 8.24 mmol, 2.5 eq.) and DMAP (0.806 g, 6.59 mmol, 2.0 eq.) were added at room temperature and stirred for 48 h under nitrogen atmosphere. The reaction mixture was treated with ice-cold water (100 mL) and extracted with ethyl acetate (3×100 mL). The combined organic layer was washed with brine (100 mL), dried over sodium sulphate, and then concentrated under reduced pressure to give (R)-7-(5-azido-4-((1-cyanoethyl)amino)pyridin-2-yl)pyrrolo[1,2-b]pyridazine-3-carbonitrile A-87 (1.1 g) as brown solid, which was directly used in the next step without further purification.

UPLC method 1: retention time: 0.996 min, [M+H]⁺=330.2.

TBM intermediates prepared using General Procedure TBM-9 are summarized in Table 22.

TABLE 22
TBM Intermediates Prepared via General Procedure TBM-9

| | Structure | Heterobicycle |
|---|---|---|
| A-57 | 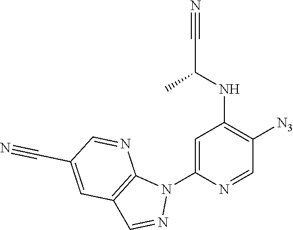 [SMILES: C[C@H](C#N)Nc1cc(-n2ncc3cc(C#N)cnc32)ncc1N=[N+]=[N-]] | 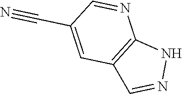 [SMILES: N#Cc1cnc2[nH]ncc2c1] |
| A-72 | 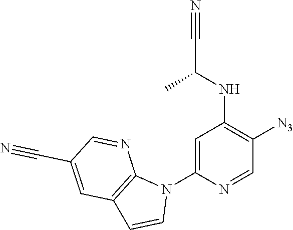 [SMILES: C[C@H](C#N)Nc1cc(-n2ccc3cc(C#N)cnc32)ncc1N=[N+]=[N-]] | 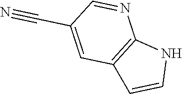 [SMILES: N#Cc1cnc2[nH]ccc2c1] |
| A-86 | 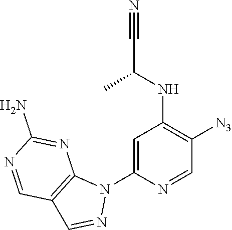 [SMILES: C[C@H](C#N)Nc1cc(-n2ncc3cnc(N)nc32)ncc1N=[N+]=[N-]] | 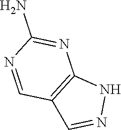 |
| A-57-I | 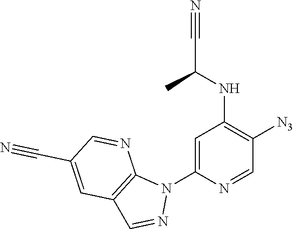 [SMILES: C[C@@H](C#N)Nc1cc(-n2ncc3cc(C#N)cnc32)ncc1N=[N+]=[N-]] | 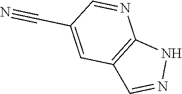 [SMILES: N#Cc1cnc2[nH]ncc2c1] |
| | *Synthesized with (2S)-2-aminopropanamide to afford the enatiomer | |
| A-83 | 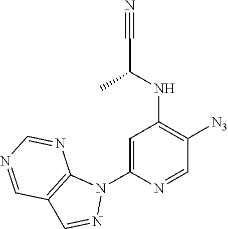 [SMILES: C[C@H](C#N)Nc1cc(-n2ncc3cncnc32)ncc1N=[N+]=[N-]] | 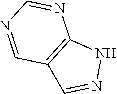 |
| A-87 | 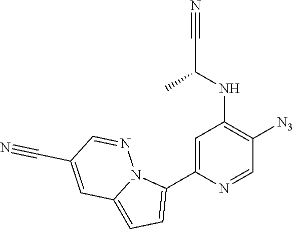 [SMILES: C[C@H](C#N)Nc1cc(-c2ccc3cc(C#N)cnn23)ncc1N=[N+]=[N-]] | 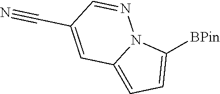 |

General Procedure TBM-10. The following scheme shows the synthetic route for General Procedure TBM-10.

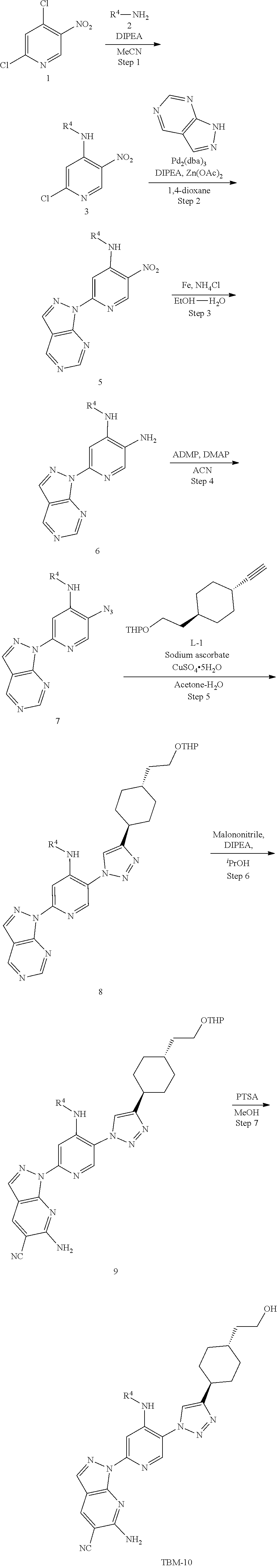

wherein R⁴ is as defined in Formula (I′) or (I).

Step 1. Preparation of 2-chloro-N-(alkyl)-5-nitropyridin-4-amine 3: To a solution of 2,4-dichloro-5-nitropyridine 1 (1.0 eq.) and alkyl amine 2′ (3.0 eq.) in acetonitrile (1.2 M) at RT under nitrogen atmosphere was added DIPEA (4 eq.) and stirred for 5 min at RT. The reaction mixture was heated to 40° C. and stirred. The reaction mixture was cooled to RT, concentrated under reduced pressure, treated with water, and extracted with EtOAc. Organic layers were washed with brine solution, dried over anhydrous Na₂SO₄, and concentrated under reduced pressure to give the crude product, which was triturated with pentane to give product 3.

Step 2. C—N coupling to afford 5: To a stirred solution of compound 3 (10.0 g, 44.1 mmol) in dioxane (0.5 M) was added 1H-pyrazolo[3,4-d]pyrimidine 4′ (1.1 eq.), zinc acetate (1.0 eq.), and DIPEA (5.0 eq.) at RT under nitrogen atmosphere. Pd₂(dba)₃ (0.05 eq.) was added and degassed the reaction mixture for another 15 min. The reaction mixture was stirred at 85° C. The reaction mixture was cooled to RT and filtered through celite bed, and concentrated under reduced pressure to give the crude product. The crude was purified by flash column chromatography on silica with 10% methanol/DCM to afford compound 5.

Step 3. Nitro reduction to afford 6: To a stirred solution of 5 (1.0 eq.) in a 4:1 mixture of ethanol/water (0.15 M) at RT, iron (2.5 eq.) and ammonium chloride (2.5 eq.) were added and stirred at 85° C. The reaction mixture was cooled to RT, treated with ice-cold saturated sodium bicarbonate solution and extracted with 20% methanol in DCM. The combined organic layer was dried over sodium sulphate and concentrated under reduced pressure to give the crude compound 6 that was used in the next step without further purification.

Step 4. Azidization to afford 7: To a stirred solution of 6 (1.0 eq.) in acetonitrile (0.3 M), ADMP (2.5 eq.) and DMAP (1.5 eq.) were added at room temperature and stirred under nitrogen atmosphere. The reaction mixture was treated with ice-cold water and extracted with ethyl acetate. The combined organic layer was washed with brine, dried over sodium sulphate and then concentrated under reduced pressure to give compound 7 that was directly used in the next step without further purification.

Step 5. 3+2 cyclization to afford triazole 8: To a stirred solution of 7 (4.0 g, 13.06 mmol) in of 11:1 acetone/water (0.25 M), 2-(2-((1r,4r)-4-ethynylcyclohexyl)ethoxy)tetrahydro-2H-pyran L-1 (1.1 eq.), sodium ascorbate (0.5 eq.) and copper(II) sulphate pentahydrate (0.5 eq.) were added at room temperature. The reaction mixture was stirred at room temperature. The reaction mixture was treated with water and extracted with ethyl acetate. The combined organic layer was dried over sodium sulphate and concentrated under reduced pressure to give the crude product. The crude product was purified by column chromatography using silica gel and ethyl acetate in pet ether to give compound 8.

Step 6. Amination cascade to afford aniline 9: To a stirred solution of triazole 8 (1.0 eq.) in 2-propanol (0.2 M), DIPEA (8.0 eq.) was added at RT. Reaction mixture was warmed up to 80° C. and stirred for 10 min and then malononitrile (5.0 eq.) was added at the same temperature and stirred at 80° C. The reaction mixture was cooled to RT, filtered, the solid was washed with 2-propanol and dried to give the crude compound aniline 9 that was directly used in the next step without further purification.

Step 7. Deprotection to afford alcohol TBM-10: To a stirred solution of alcohol 9 (1.0 eq.) in MeOH, pTSA (0.5 eq.) was added at RT and stirred for 1 h. The reaction mixture was diluted with 20% methanol in DCM and treated with saturated sodium bicarbonate solution. The organic layers were collected, washed with brine, dried over sodium sulphate, and concentrated under reduced pressure to give crude product. The crude was washed with MTBE to obtain the compound TBM-10.

### Example S33. 6-amino-1-(4-(((R)-1-cyanoethyl)amino)-5-(4-((1r,4R)-4-(2-hydroxyethyl)cyclohexyl)-1H-1,2,3-triazol-1-yl)pyridin-2-yl)-1H-pyrazolo[3,4-b]pyridine-5-carbonitrile (A-66)

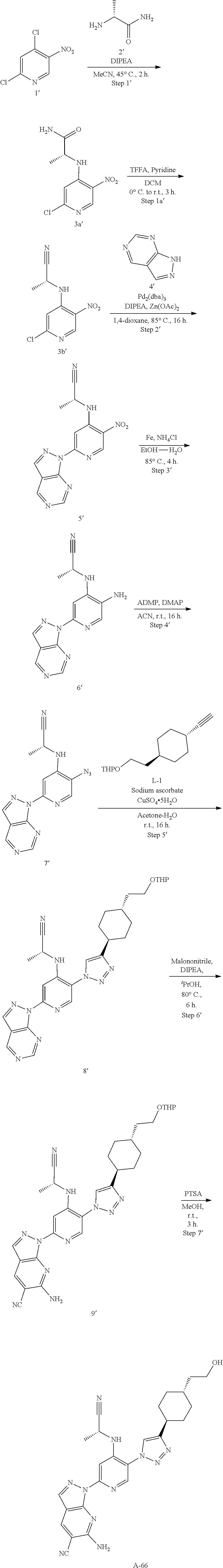

LCMS Method 1. Kinetex XB-C18, 75×3.0 mm, 2.6 Temperature: RT, Flow: 1.0 mL/min, Run Time: 5 minutes, Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 100% Mobile Phase B for 4.0 min. MSD positive.

LCMS Method 2. Aquity Uplc BEH-C18, 50×3.0 mm, 1.7 Temperature: RT, Flow: 1.0 mL/min, Run Time: 5 minutes, Mobile Phase Conditions: Mobile Phase-A: 0.1% TFA in H₂O, Mobile Phase-B: 0.1% TFA in ACN, Gradient: Initial 80% Mobile Phase A and 20% Mobile Phase B linear gradient to 100% Mobile Phase B for 3.0 min. MSD positive.

Step 1′. Preparation of (R)-2-((2-chloro-5-nitropyridin-4-yl)amino)propenamide 3a: To a stirred solution of 2,4-dichloro-5-nitropyridine 1′ (50.0 g, 259 mmol) in acetonitrile (500 mL) was added (R)-2-aminopropanamide hydrochloride 2′ (38.7 g, 311 mmol) and DIPEA (113.0 mL, 648 mmol) at RT, under nitrogen atmosphere and the resulting solution was stirred for 2 h at 45° C. The solution was then cooled to room temperature and concentrated under reduced pressure (70% volume). Finally, cold water (500 mL) was added to the reaction mixture and stirred for 10 min to afford a pale yellow solid. The solid was filtered, washed with water and dried to give the pure compound (R)-2-((2-chloro-5-nitropyridin-4-yl)amino)propanamide 3a′ (55.0 g, 86% yield) as a pale yellow solid.

LCMS method 1: retention time 0.88 min, 99.29% purity at 220 nm, [M+H]⁺=245.0

Step 1a′. Preparation of (R)-2-((2-chloro-5-nitropyridin-4-yl)amino)propanenitrile 3b′: To a stirred solution of (R)-2-((2-chloro-5-nitropyridin-4-yl) amino) propanamide 3a′ (54.0 g, 221 mmol) in DCM (500 mL) at 0° C. was added pyridine (179.0 mL, 2207 mmol) slowly over 10 min. Then, TFAA (156.0 mL, 1104 mmol) was added in a drop-wise manner (over 30 min) at 0° C. and stirred at RT for 3 h. The reaction mixture was cooled to 0° C. and treated with ice-cold water (500 mL). The reaction mixture was extracted with DCM (3×300 mL), the combined organic layer was washed with 1.0 N HCl (200 mL), followed by brine solution (500 mL), dried over sodium sulphate, and concentrated under reduced pressure to give the crude product. The crude product was washed with MTBE (3×200 mL) to afford pale yellow solid and dried to give pure compound (R)-2-((2-chloro-5-nitropyridin-4-yl)amino)propanenitrile 3b′ (46.0 g, 92% yield) as pale-yellow solid.

LCMS method 1: retention time 1.75 min, 99.94% purity at 220 nm, [M−H]⁺=225.0

Step 2′. Preparation of (R)-2-((5-nitro-2-(1H-pyrazolo[3,4-d]pyrimidin-1-yl)pyridin-4-yl)amino)propanenitrile 5′: To a stirred solution of (R)-2-((2-chloro-5-nitropyridin-4-yl) amino) propanenitrile 3b′ (10.0 g, 44.1 mmol) in dioxane (80 mL) was added 1H-pyrazolo[3,4-d]pyrimidine 4′ (5.83 g, 48.5 mmol), zinc acetate (8.10 g, 44.1 mmol), and DIPEA (46.2 mL, 265 mmol) at RT under nitrogen atmosphere. Pd₂(dba)₃ (2.02 g, 2.206 mmol) was added and degassed the reaction mixture for another 15 min. The reaction mixture was stirred at 85° C. for 16 h. The reaction mixture was cooled to RT and filtered through celite bed, and concentrated under reduced pressure to give the crude product. The crude was purified by flash column chromatography on silica with 10% methanol/DCM to afford compound (R)-2-((5-nitro-2-(1H-pyrazolo[3,4-d]pyrimidin-1-yl)pyridin-4-yl)amino)propanenitrile 5′ (7.0 g, 48.3% yield) as a yellow solid.

LCMS method 2: retention time 2.33 min, 94.01% purity at 220 nm, [M+H]⁺=311

Step 3′. Preparation of (R)-2-((5-amino-2-(1H-pyrazolo[3,4-d]pyrimidin-1-yl)pyridin-4-yl)amino)propanenitrile 6′: To a stirred solution of (R)-2-((5-nitro-2-(1H-pyrazolo[3,4-d]pyrimidin-1-yl)pyridin-4-yl)amino)propanenitrile 5′ (7.0 g, 22.56 mmol) in 150 mL of ethanol:water (4:1) at RT, iron (3.15 g, 56.4 mmol) and ammonium chloride (3.02 g, 56.4 mmol) were added and stirred for 4 h at 85° C. The reaction mixture was cooled to RT, treated with ice-cold saturated sodium bicarbonate (50 mL) solution and extracted with 20% methanol in DCM (3×200 mL). The combined organic layer was dried over sodium sulphate and concentrated under reduced pressure to give the crude compound (R)-2-((5-amino-2-(1H-pyrazolo[3,4-d]pyrimidin-1-yl)pyridin-4-yl)amino)propanenitrile 6′ (3.0 g), which was used in the next step without further purification.

LCMS method 1: retention time 2.20 min, [M+H]⁺=281.2

Step 4′. Preparation of (R)-2-((5-azido-2-(1H-pyrazolo[3,4-d]pyrimidin-1-yl)pyridin-4-yl)amino)propanenitrile 7′: To a stirred solution of (R)-2-((5-amino-2-(1H-pyrazolo[3,4-d]pyrimidin-1-yl) pyridin-4-yl) amino) propane nitrile 6′ (3.0 g, 10.70 mmol) in acetonitrile (30 mL), ADMP (2.195 g, 26.8 mmol) and DMAP (1.961 g, 16.05 mmol) were added at room temperature and stirred for 16 h under nitrogen atmosphere. The reaction mixture was treated with ice-cold water and extracted with ethyl acetate (3×100 mL). The combined organic layer was washed with brine, dried over sodium sulphate and then concentrated under reduced pressure to give compound (R)-2-((5-azido-2-(1H-pyrazolo[3,4-d]pyrimidin-1-yl)pyridin-4-yl)amino)propanenitrile 7′ (4.0 g), which was directly used in the next step without further purification.

LCMS method 1: retention time 1.15 min, [M+H]⁺=305

Step 5′. Preparation of (2R)-2-((2-(1H-pyrazolo[3,4-d]pyrimidin-1-yl)-5-(4-((1r,4R)-4-(2-((tetrahydro-2H-pyran-2-yl)oxy)ethyl)cyclohexyl)-1H-1,2,3-triazol-1-yl)pyridin-4-yl)amino)propanenitrile 8′: To a stirred solution of (R)-2-((5-azido-2-(1H-pyrazolo[3,4-d]pyrimidin-1-yl) pyridin-4-yl) amino) propane nitrile 7′ (4.0 g, 13.06 mmol) in 55.0 mL of acetone:water (11:1), 2-(2-((1r,4r)-4-ethynylcyclohexyl)ethoxy)tetrahydro-2H-pyran L-1 (3.40 g, 14.37 mmol), sodium ascorbate (0.536 g, 6.53 mmol) and copper(II) sulphate pentahydrate (1.630 g, 6.53 mmol) were added at room temperature. The reaction mixture was stirred at room temperature for 16 h. The reaction mixture was treated with water and extracted with ethyl acetate (3×40 mL). The combined organic layer was dried over sodium sulphate and concentrated under reduced pressure to give the crude product. The crude product was purified by column chromatography using silica gel (230-400 mesh) and eluted with 70-90% ethyl acetate in pet ether to give compound (2R)-2-((2-(1H-pyrazolo[3,4-d]pyrimidin-1-yl)-5-(4-((1r,4R)-4-(2-((tetrahydro-2H-pyran-2-yl)oxy)ethyl)cyclohexyl)-1H-1,2,3-triazol-1-yl)pyridin-4-yl)amino)propanenitrile 8′ (1.6 g, 18.69% yield) as yellow solid.

LCMS method 1: retention time 2.72 min, 82.87% purity at 220 nm, [M+H]⁺=543.2

Step 6′. Preparation of 6-amino-1-(4-(((R)-1-cyanoethyl)amino)-5-(4-((1r,4R)-4-(2-((tetrahydro-2H-pyran-2-yl)oxy)ethyl)cyclohexyl)-1H-1,2,3-triazol-1-yl)pyridin-2-yl)-1H-pyrazolo[3,4-b]pyridine-5-carbonitrile 9′: To a stirred solution of (2R)-2-((2-(1H-pyrazolo[3,4-d]pyrimidin-1-yl)-5-(4-((1r,4R)-4-(2-((tetrahydro-2H-pyran-2-yl) oxy) ethyl) cyclohexyl)-1H-1,2,3-triazol-1-yl) pyridin-4-yl) amino) propane nitrile 8′ (1.1 g, 2.027 mmol) in 2-propanol (10 mL), DIPEA (2.83 mL, 16.22 mmol) was added at RT. Reaction mixture was warmed up to 80° C. and stirred for 10 min and then malononitrile (0.453 mL, 10.14 mmol) was added at the same temperature and stirred for another for 6 h at 80° C. The reaction mixture was cooled to RT, filtered, the solid was washed with 2-propanol (2×10 mL) and dried to give the crude compound 6-amino-1-(4-(((R)-1-cyanoethyl)amino)-5-(4-((1r,4R)-4-(2-((tetrahydro-2H-pyran-2-yl)oxy)ethyl)cyclohexyl)-1H-1,2,3-triazol-1-yl)pyridin-2-yl)-1H-pyrazolo[3,4-b]pyridine-5-carbonitrile 9′ (1.0 g), which was directly used in the next step without further purification.

LCMS method 1: retention time 2.23 min, [M+H]⁺=582.2

Step 7′. Preparation of 6-amino-1-(4-(((R)-1-cyanoethyl)amino)-5-(4-((1r,4R)-4-(2-hydroxyethyl)cyclohexyl)-1H-1,2,3-triazol-1-yl)pyridin-2-yl)-1H-pyrazolo[3,4-b]pyridine-5-carbonitrile A-66: To a stirred solution of 6-amino-1-(4-(((R)-1-cyanoethyl) amino)-5-(4-((1r,4R)-4-(2-((tetrahydro-2H-pyran-2-yl) oxy) ethyl) cyclohexyl)-1H-1,2,3-triazol-1-yl) pyridin-2-yl)-1H-pyrazolo[3,4-b]pyridine-5-carbonitrile 9′ (800 mg, 1.375 mmol) in MeOH, pTSA (131 mg, 0.688 mmol) was added at RT and stirred for 1 h. The reaction mixture was diluted with 20% methanol in DCM (50 mL) and treated with saturated sodium bicarbonate solution (20 mL). The organic layers were collected, washed with brine, dried over sodium sulphate, and concentrated under reduced pressure to give crude product. The crude was washed with MTBE (3×20 mL) to obtain the compound 6-amino-1-(4-(((R)-1-cyanoethyl)amino)-5-(4-((1r,4R)-4-(2-hydroxyethyl)cyclohexyl)-1H-1,2,3-triazol-1-yl)pyridin-2-yl)-1H-pyrazolo[3,4-b]pyridine-5-carbonitrile A-66 (450 mg, 61.1% yield).

LCMS method 1: retention time 1.57 min, 92.93% purity at 220 nm, [M+H]⁺=498.2.

TBM intermediates prepared using General Procedure TBM-10 are summarized in Table 23.

TABLE 23
TBM Intermediates Prepared via General Procedure TBM-10

| | Structure | Amine |
|---|---|---|
| A-64 | 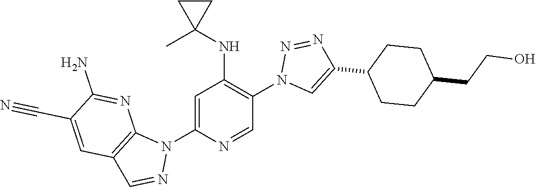 | 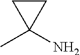 |
| A-67 | 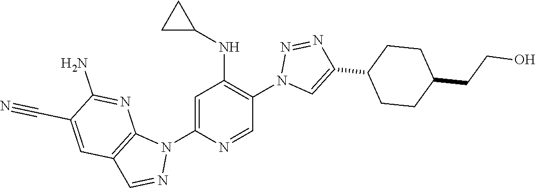 | 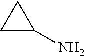 |
| A-82 | 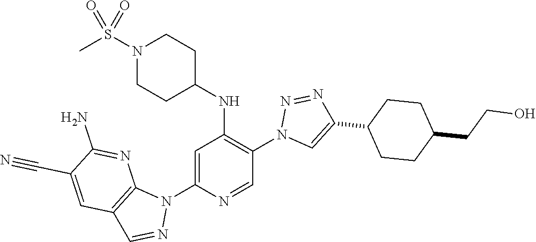 | 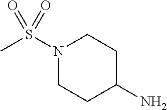 |
| A-66 | 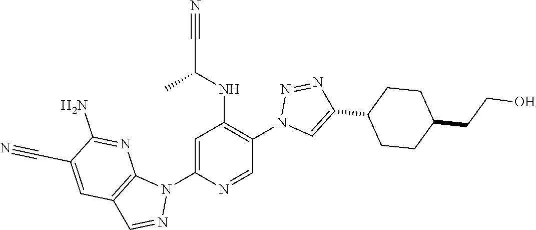 | |
| A-69 | 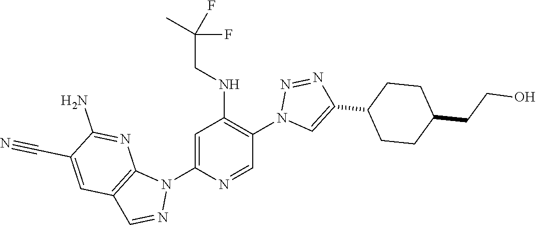 | 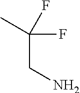 |

General Procedure TBM-11. The scheme shown below for the synthesis of A-77 is provided as a representative synthesis for General Procedure TBM-11.

### Example S34. (R)-1-(4-((1-cyanoethyl)amino)-5-ethynylpyridin-2-yl)-1H-pyrazolo[3,4-b]pyridine-5-carbonitrile (A-77)

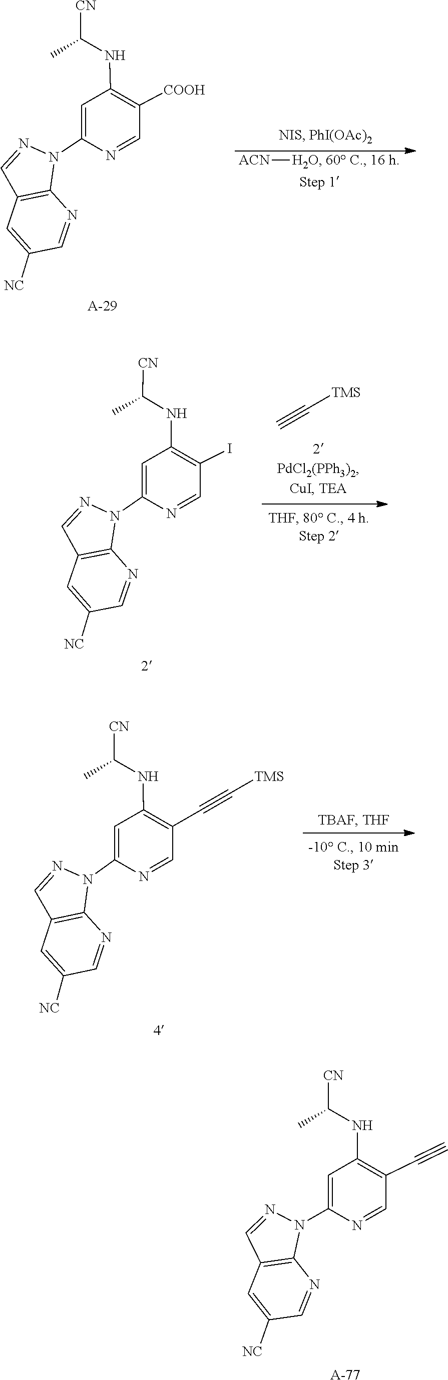

LCMS Method 1. X-Bridge C-8, 50×4.6 mm, 5.0 μm, Temperature: RT, Flow: 1.5 mL/min, Run Time: 5 minutes, Mobile Phase Conditions: Mobile Phase-A: 0.1% TFA in H₂O, Mobile Phase-B: 0.1% TFA in ACN, Gradient: Initial 95% Mobile Phase A and 5% Mobile Phase B linear gradient to 5% Mobile phase A and 95% Mobile Phase B for 2.5 min. MSD positive.

LCMS Method 2. Kinetex XB C-18, 75×3.0 mm, 2.6 Temperature: RT, Flow: 1.0 mL/min, Run Time: 5 minutes, Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 100% Mobile Phase B for 4.0 min. MSD positive.

Step 1′ Preparation of (R)-1-(4-((1-cyanoethyl)amino)-5-iodopyridin-2-yl)-1H-pyrazolo[3,4-b]pyridine-5-carbonitrile 2′: To a solution of (R)-6-(5-cyano-1H-pyrazolo[3,4-b]pyridin-1-yl)-4-((1-cyanoethyl)amino)nicotinic acid pivalate A-29 (600 mg, 1.584 mmol) in acetonitrile/water (1.6/0.8 mL, 0.660 M) was added N-iodosuccinimide (428 mg, 1.901 mmol). The reaction was stirred at 60° C. for 30 min and then (Diacetoxyiodo)benzene (306 mg, 0.950 mmol) was added slowly under nitrogen. The reaction mixture was stirred at 60° C. for 16 h. The reaction mixture was cooled to RT and treated with water (2.0 mL). The reaction mixture was extracted with ethyl acetate (3×3.0 mL). The combined organic layer was washed with brine solution, dried over sodium sulphate, and concentrated under reduced pressure to give the crude product. The crude was purified by column chromatography using silica gel (100-200 mesh) with 40-50% ethyl acetate in pet ether to give a pure compound (R)-1-(4-((1-cyanoethyl)amino)-5-iodopyridin-2-yl)-1H-pyrazolo[3,4-b]pyridine-5-carbonitrile 2′ (390 mg, 47% yield) as a white semi solid.

LCMS method 1: retention time: 1.967 min, 80% purity at 220 nm, [M+H]⁺=416.1

Step 2′. Preparation of (R)-1-(4-((1-cyanoethyl)amino)-5-((trimethylsilyl)ethynyl)pyridin-2-yl)-1H-pyrazolo[3,4-b]pyridine-5-carbonitrile 4′: To a stirred solution of (R)-1-(4-((l-cyanoethyl)amino)-5-iodopyridin-2-yl)-1H-pyrazolo[3,4-b]pyridine-5-carbonitrile 2′ (900 mg, 2.168 mmol) in tetrahydrofuran (5.0 mL) was added ethynyltrimethylsilane 3′ (0.463 mL, 3.25 mmol), copper (I) Iodide (413 mg, 2.168 mmol), and TEA (0.604 mL, 4.34 mmol). This resulting mixture was degassed for 10 min and then was added dichlorobis(triphenyl phosphine)palladium(II) (152 mg, 0.217 mmol). The resulting solution was stirred for 4 h at 80° C. The reaction mixture was cooled to RT and diluted with ice-cold water, and extracted with ethyl acetate (3×20 mL). The combined organic layer was washed with brine, dried over sodium sulphate and then concentrated under reduced pressure to give crude product (R)-1-(4-((l-cyanoethyl)amino)-5-((trimethylsilyl)ethynyl)pyridin-2-yl)-1H-pyrazolo[3,4-b]pyridine-5-carbonitrile 4′ (900 mg, 81% yield). The crude was used in the next step without further purification.

LCMS method 2: retention time 2.72 min, [M+H]⁺=386.2

Step 3′. Preparation (R)-1-(4-((1-cyanoethyl)amino)-5-ethynylpyridin-2-yl)-1H-pyrazolo[3,4-b]pyridine-5-carbonitrile A-77: To a solution of (R)-1-(4-((1-cyanoethyl)amino)-5-((trimethylsilyl)ethynyl)pyridin-2-yl)-1H-pyrazolo[3,4-b]pyridine-5-carbonitrile 4′ (900 mg, 2.335 mmol) in THF (5.0 mL) was added TBAF (4.67 mL, 4.67 mmol) at −10° C. and was allowed to stir at same temperature for 10 min. The reaction mixture was quenched with ice-cold ammonium chloride solution and extracted with ethyl acetate (3×20 mL). The combined organic layer was washed with brine, dried over sodium sulphate and then concentrated under reduced pressure to give crude product (R)-1-(4-((1-cyanoethyl)amino)-5-ethynylpyridin-2-yl)-1H-pyrazolo[3,4-b]pyridine-5-carbonitrile A-77 (400 mg), which was used in the next step without further purification.

LCMS method 2: retention time: 1.22 min, [M+H]⁺=314.0.

TBM intermediates prepared using General Procedure TBM-11 are summarized in Table 24.

TABLE 24
TBM Intermediates Prepared via General Procedure TBM-11

| | Structure |
|---|---|
| A-77 | 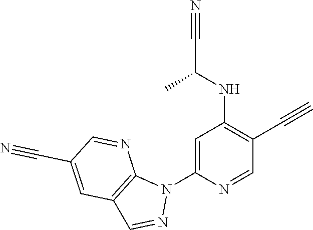 [SMILES: C#Cc1cnc(-n2ncc3cc(C#N)cnc32)cc1N[C@H](C)C#N] |

General Procedure TBM-12. The scheme shown below for the synthesis of A-78 is provided as a representative synthesis for General Procedure TBM-12.

### Example S35. 1-(4-(((R)-1-cyanoethyl)amino)-5-(5-((1r,4R)-4-(2-hydroxyethyl)cyclohexyl)isoxazol-3-yl)pyridin-2-yl)-1H-pyrazolo[3,4-b]pyridine-5-carbonitrile (A-78)

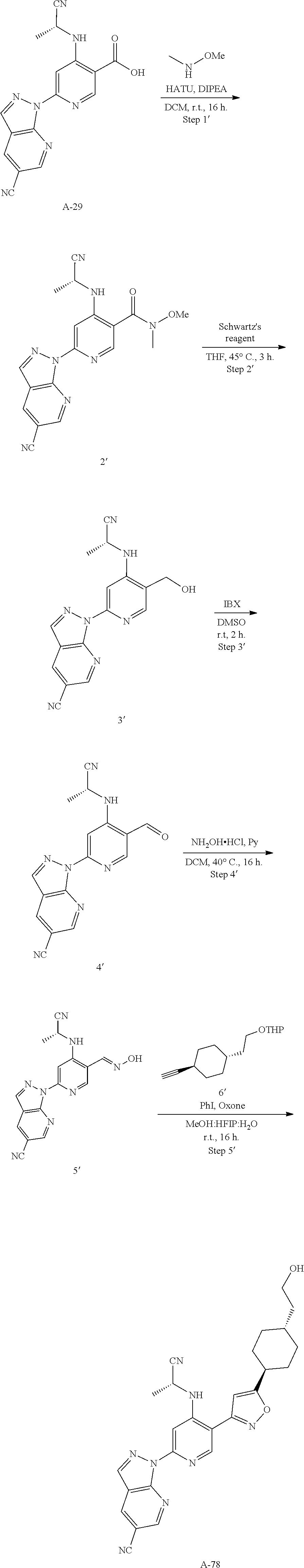

LCMS Method 1. Kinetex XB C-18, 75×3.0 mm, 2.6 Temperature: RT, Flow: 1.0 mL/min, Run Time: 5 minutes, Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 100% Mobile Phase B for 4.0 min. MSD positive.

LCMS Method 2. X-Bridge C-18, 50×4.6 mm, 5.0 μm, Temperature: RT, Flow: 1.0 mL/min, Run Time: 5 minutes, Mobile Phase Conditions: Mobile Phase-A: 0.1% TFA in H₂O, Mobile Phase-B: 0.1% TFA in ACN, Gradient: Initial 95% Mobile Phase A and 5% Mobile Phase B linear gradient to 5% Mobile phase A and 95% Mobile Phase B for 2.5 min. MSD positive.

Step 1′. Preparation of (R)-6-(5-cyano-1H-pyrazolo[3,4-b]pyridin-1-yl)-4-((1-cyano ethyl) amino)-N-methoxy-N-methyl nicotinamide 2′: To a solution of (R)-6-(5-cyano-1H-pyrazolo[3,4-b]pyridin-1-yl)-4-((1-cyanoethyl)amino)nicotinic acid A-29 (1.0 g, 3.00 mmol) in DCM (10 mL) was added N,O-dimethyl hydroxylamine hydrochloride (0.293 g, 3.00 mmol), and DIPEA (2.096 mL, 12.00 mmol) at RT. This resulting solution was stirred for 5-10 min, and then was added HATU (2.282 g, 6.00 mmol). The resulting solution was stirred for 16 h at RT. The reaction mixture was diluted with ice-cold water and extracted with ethyl acetate (3×20 mL). The combined organic layer was washed with brine, dried over sodium sulphate, and then concentrated under reduced pressure to give the crude product. The crude product was purified by column chromatography using silica gel (100-200 mesh) with 0-5% methanol in DCM to give pure (R)-6-(5-cyano-1H-pyrazolo[3,4-b]pyridin-1-yl)-4-((1-cyano ethyl) amino)-N-methoxy-N-methyl nicotinamide 2′ (1.0 g, 81% yield) as an off-white solid.

LCMS method 1: retention time 1.44 min, 91.88% purity at 220 nm, [M+H]⁺=377.2.

Step 2′. Preparation (R)-1-(4-((1-cyanoethyl) amino)-5-formylpyridin-2-yl)-1H-pyrazolo[3,4-b]pyridine-5-carbonitrile 3′: To a solution of (R)-6-(5-cyano-1H-pyrazolo[3,4-b]pyridin-1-yl)-4-((1-cyanoethyl)amino)-N-methoxy-N-methylnicotinamide 2′ (1.0 g, 2.66 mmol) in dry THF was added Schwartz's reagent (1.370 g, 5.31 mmol) at RT. The resulting solution was stirred for 3 h at 45° C. The reaction mixture was cooled to RT and diluted with ice-cold water, and extracted with ethyl acetate (3×20 mL). The combined organic layer was washed with brine, dried over sodium sulphate, and then concentrated under reduced pressure to give the crude product. The crude product was purified by column chromatography using silica gel (100-200 mesh) with 5-10% methanol in DCM to give compound (R)-1-(4-((1-cyanoethyl) amino)-5-formylpyridin-2-yl)-1H-pyrazolo[3,4-b]pyridine-5-carbonitrile 4′ (200 mg, 0.555 mmol, 21% yield) as an off-white solid and (R)-1-(4-((1-cyanoethyl)amino)-5-(hydroxymethyl)pyridin-2-yl)-1H-pyrazolo[3,4-b]pyridine-5-carbonitrile 3′ (500 mg, 1.315 mmol, 50% yield) as a pale yellow solid.

LCMS method 2: retention time: 1.537 min, 83.98% purity at 220 nm, [M+H]⁺=320.1.

¹H NMR (400 MHz, DMSO-d6): δ ppm 1.65 (d, J=6.8 Hz, 3H), 4.57 (s, 2H), 4.87-4.95 (m, 1H), 5.38 (brs, 1H), 6.59 (d, J=8.0 Hz, 1H), 7.29 (s, 1H), 8.21 (s, 1H), 8.64 (s, 1H), 9.01-9.03 (m, 2H).

Step 3′. Preparation of (R)-1-(4-((1-cyanoethyl) amino)-5-formylpyridin-2-yl)-1H-pyrazolo[3,4-b]pyridine-5-carbonitrile 4′: To a stirred solution of (R)-1-(4-((l-cyanoethyl)amino)-5-(hydroxymethyl)pyridin-2-yl)-1H-pyrazolo[3,4-b]pyridine-5-carbonitrile 3′ (300 mg, 0.939 mmol) in DMSO (3 mL) was added IBX (789 mg, 2.82 mmol) at RT. This resulting solution was stirred at RT for 3 h. The reaction mixture was diluted with ice-cold water and extracted with ethyl acetate (3×50 mL). The combined organic layer was washed with brine, dried over sodium sulphate and then concentrated under reduced pressure to give the crude product (R)-1-(4-((l-cyanoethyl)amino)-5-formylpyridin-2-yl)-1H-pyrazolo[3,4-b]pyridine-5-carbonitrile 4′ (250 mg, 77% yield).

LCMS method 1: retention time: 1.39 min, 91.74% purity at 220 nm, [M+H]⁺=318.0.

¹H NMR (400 MHz, DMSO-d6): δ ppm 1.72 (d, J=7.2 Hz, 3H), 5.10-5.17 (m, 1H), 7.65 (s, 1H), 8.72-8.74 (m, 2H), 8.85 (s, 1H), 9.05-9.11 (m, 2H), 10.02 (s, 1H).

Step 4′. Preparation of (R,E)-1-(4-((1-cyanoethyl)amino)-5-((hydroxyimino)methyl)pyridin-2-yl)-1H-pyrazolo[3,4-b]pyridine-5-carbonitrile 5′: To a solution of (R)-1-(4-((1-cyanoethyl)amino)-5-formylpyridin-2-yl)-1H-pyrazolo[3,4-b]pyridine-4′ (350 mg, 1.103 mmol) in DCM (10 mL) was added hydroxylamine hydrochloride (115 mg, 1.655 mmol) and pyridine (0.178 mL, 2.206 mmol) at RT. This resulting mixture was stirred at 40° C. for 16 h. The reaction mixture was cooled to RT and was quenched with ice old citric acid solution and extracted with ethyl acetate (3×20 mL). The combined organic layer was washed with brine, dried over sodium sulphate and then concentrated under reduced pressure to give the crude product (R,E)-1-(4-((1-cyanoethyl)amino)-5-((hydroxyimino)methyl)pyridin-2-yl)-1H-pyrazolo[3,4-b]pyridine-5-carbonitrile 5′ (300 mg, 72% yield), which was used in the next step without further purification.

LCMS method 1: retention time 1.274 min, 88.30% purity at 220 nm, [M+H]⁺=333.2.

¹H NMR (400 MHz, DMSO-d6): δ ppm 1.70 (d, J=7.2 Hz, 3H), 5.01-5.08 (m, 1H), 7.49 (s, 1H), 8.19 (d, J=7.2 Hz, 1H), 8.44 (s, 1H), 8.45 (s, 1H), 8.69 (s, 1H), 9.04-9.05 (m, 2H), 11.60 (s, 1H).

Step 5′. Preparation of 1-(4-(((R)-1-cyanoethyl)amino)-5-(54(1r,4R)-4-(2-hydroxyethyl)cyclohexyl)isoxazol-3-yl)pyridin-2-yl)-1H-pyrazolo[3,4-b]pyridine-5-carbonitrile A-78: To a stirred solution of (R,E)-1-(4-((1-cyanoethyl)amino)-5-((hydroxyimino)methyl)pyridin-2-yl)-1H-pyrazolo[3,4-b]pyridine-5-carbonitrile 5′ (300 mg, mmol) in MeOH (2.0 mL), HFIP (2.0 mL), and Water (0.2 mL) was added 2-(2-((1r,4r)-4-ethynylcyclohexyl)ethoxy)tetrahydro-2H-pyran 6′ (213 mg, 0.903 mmol), oxone (1665 mg, 2.71 mmol), and phenyl iodide (36.8 mg, 0.181 mmol) at RT. This resulting solution was stirred at RT for 16 h. The reaction mixture was diluted with ice-cold water and extracted with ethyl acetate (3×20 mL). The combined organic layer was washed with brine, dried over sodium sulphate, and then concentrated under reduced pressure to give the crude product. The crude product was purified by column chromatography using silica gel (100-200 mesh) with 50-80% ethyl acetate in pet ether to give pure 1-(4-(((R)-1-cyanoethyl)amino)-5-(5-((1r,4R)-4-(2-hydroxyethyl)cyclohexyl)isoxazol-3-yl)pyridin-2-yl)-1H-pyrazolo[3,4-b]pyridine-5-carbonitrile A-78 (100 mg, 13% yield) as a pale yellow brown solid.

LCMS method 1: retention time: 2.65 min, 57.18% purity at 220 nm, [M+H]⁺=483.2.

TBM intermediates prepared using General Procedure TBM-12 are summarized in Table 25.

TABLE 25
TBM Intermediates Prepared via General Procedure TBM-12

| | Structure |
|---|---|
| A-78 | 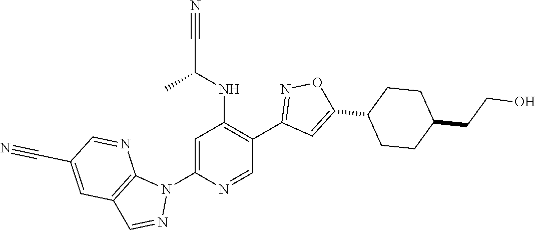 |

General Procedure TBM-13. The scheme shown below for the synthesis of A-80 is provided as a representative synthesis for General Procedure TBM-13.

### Example S36. 1-(4-(((R)-1-cyanoethyl)amino)-5-(1-((1r,4R)-4-(2-hydroxyethyl)cyclohexyl)-1H-pyrazol-4-yl)pyridin-2-yl)-1H-pyrazolo[3,4-b]pyridine-5-carbonitrile (A-80)

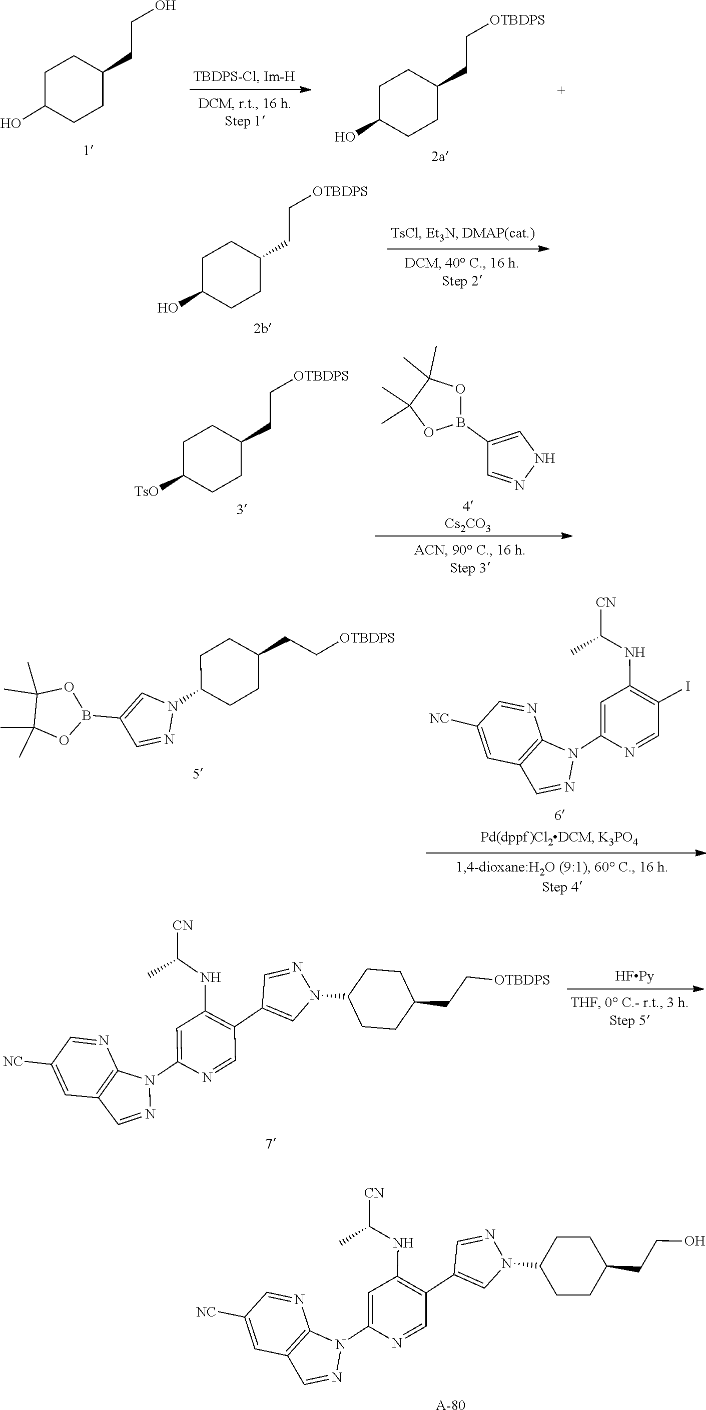

UPLC Method 1. Column: Kinetex XB-C18, 75×3.0 mm, 2.6 Temperature: RT, Flow: 1.0 mL/min, Run Time: 5 minutes, Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 100% Mobile Phase B for 4.0 min. MSD positive

LCMS Method 2. Column: Kinetex XB-C18, 50×4.6 mm, 5 μm, Temperature: RT, Flow: 1.5 mL/min, Run Time: 6 min, Mobile phase-A: 0.1% TFA in H₂O, Mobile phase-B: 0.1% TFA in CH₃CN, Gradient: Initial 95% Mobile Phase A and 5% Mobile Phase B linear gradient to 95% Mobile Phase B for 2.5 min. MSD positive.

Step 1′. Preparation of (1s,4s)-4-(2-((tert-butyldiphenylsilyl)oxy)ethyl)cyclohexan-1-ol 2a′ and (1r,4r)-4-(2-((tert-butyldiphenylsilyl)oxy)ethyl)cyclohexan-1-ol 2b′: To a cooled and stirred solution of 4-(2-hydroxyethyl)cyclohexan-1-ol 1′ (8.5 g, 58.9 mmol, 1.0 eq.) in DCM (100 ml) was added 1-methylimidazole (9.40 mL, 118 mmol, 2.0 eq.) followed by tert-butyldiphenylchlorosilane (19.68 mL, 77 mmol, 1.3 eq.), and stirred the reaction at RT for 16 h. The reaction mixture was cooled to 0° C. and treated with ice-cold saturated sodium bicarbonate (50 mL) solution and extracted with DCM (3×100 mL). The combined organic layer was dried over sodium sulphate, filtered and concentrated under reduced pressure to give the crude compound. The crude was purified by flash column chromatography on silica with 5% EtOAc/Pet ether to afford the desired isomer (1s,4s)-4-(2-((tertbutyldiphenylsilyl)oxy)ethyl)cyclohexan-1-ol 2a′ (4.7 g, 11.67 mmol, 19.80% yield) and (1r,4r)-4-(2-((tert-butyldiphenylsilyl)oxy)ethyl)cyclohexan-1-ol 2b′ (4.5 g, 19.95% yield) as a mixture of isomers.

¹H NMR (400 MHz, DMSO-d6): δ ppm 1.00 (s, 9H), 1.30-1.36 (m, 5H), 1.45-1.50 (m, 6H), 3.66-3.69 (m, 3H), 4.20 (brs, 1H), 7.42-7.47 (m, 6H), 7.60-7.62 (m, 4H).

Step 2′. Preparation of (1s,4s)-4-(2-((tertutyldiphenylsilyl) oxy) ethyl) cyclohexyl 4-methylbenzenesulfonate 3′: To a stirred and cooled solution of (1s,4s)-4-(2-((tert-butyldiphenylsilyl)oxy)ethyl)cyclohexan-1-ol 2a′ (2.0 g, 5.23 mmol, 1.0 eq.) in DCM (20 mL) was added TEA (2.186 mL, 15.68 mmol, 3.0 eq.) followed by tosyl chloride (1.49 g, 7.84 mmol, 1.5 eq.) and cat. DMAP (0.032 g, 0.261 mmol, 0.05 eq.). The reaction mixture was stirred at 40° C. for 16 h. The reaction mixture was cooled to 0° C. and treated with ice-cold saturated sodium bicarbonate (50 mL) solution and extracted with DCM (3×80 mL). The combined organic layer was dried over sodium sulphate, filtered and concentrated under reduced pressure to give the crude compound. The crude product was purified by column chromatography using silica gel (230-400 mesh) 5% EtOAc/pet ether to afford (1s,4s)-4-(2-((tert-butyldiphenylsilyl)oxy)ethyl)cyclohexyl 4-methylbenzenesulfonate 3′ (1.9 g, 64.3% yield) as a white solid.

¹H NMR (400 MHz, DMSO-d6): δ ppm 1.05 (s, 9H), 1.12-1.22 (m, 2H), 1.38-1.44 (m, 7H), 1.60-1.63 (m, 2H), 2.42 (s, 3H), 3.66 (t, J=5.60 Hz, 2H), 4.62-4.67 (m, 1H), 7.41-7.48 (m, 8H), 7.59-7.61 (m 4H), 7.77-7.79 (m, 2H).

Step 3′. Preparation of 14(1r,4r)-4-(2-((tert-butyldiphenylsilyl)oxy)ethyl)cyclohexyl)-4-(4,4,5,5-tetramethyl-1,3,2-dioxaborolan-2-yl)-1H-pyrazole 5′: To a stirred solution of 4-(4,4,5,5-tetramethyl-1,3,2-dioxaborolan-2-yl)-1H-pyrazole 4′ (1.0 g, 5.15 mmol, 1.0 eq.) and (1s,4s)-4-(2-((tert-butyldiphenylsilyl)oxy)ethyl)cyclohexyl 4-methylbenzenesulfonate 3′ (2.77 g, 5.15 mmol, 1.0 eq.) in MeCN (10 mL) at room temperature was added cesium carbonate (5.04 g, 15.46 mmol, 3.0 eq.). The reaction mixture was heated to 90° C. and stirred for 16 h. The reaction mixture cooled to room temperature, filtered through the celite bed, and washed with EtOAc (3×30 mL). The combined organic phases were washed with brine (20 mL), dried over magnesium sulphate, filtered and concentrated under reduced pressure. The crude product was purified by column chromatography using silica gel (230-400 mesh) 10% EtOAc/pet ether to afford 1-((1r,4r)-4-(2-((tertbutyldiphenylsilyl)oxy)ethyl)cyclohexyl)-4-(4,4,5,5-tetramethyl-1,3,2-dioxaborolan-2-yl)-1H-pyrazole 5′ (480 mg, 14% yield) as a colourless oil.

UPLC method 1: retention time: 3.20 min, 84.9% purity at 220 nm, [M+H]⁺=559.4.

Step 4′. Preparation of 1-(5-(1-((1r,4R)-4-(2-((tert-butyldiphenylsilyl)oxy)ethyl)cyclohexyl)-1H-pyrazol-4-yl)-4-(((R)-1-cyanoethyl) amino)pyridin-2-yl)-1H-pyrazolo[3,4-b]pyridine-5-carbonitrile 7′: To a stirred solution of 1-((1r,4r)-4-(2-((tert-butyldiphenylsilyl)oxy)ethyl)cyclohexyl)-4-(4,4,5,5-tetramethyl-1,3,2-dioxaborolan-2-yl)-1H-pyrazole 5′ (538 mg, 0.963 mmol, 1.0 eq.) and (R)-1-(4-((1-cyanoethyl)amino)-5-iodopyridin-2-yl)-1H-pyrazolo[3,4-b]pyridine-5-carbonitrile 6′ (400 mg, 0.963 mmol, 1.0 eq.) in 9:1 ratio of 1,4-dioxane:water (10 mL) at room temperature was added potassium phosphate tribasic (613 mg, 2.89 mmol 3.0 eq.) and purged with N₂ gas for 20 min. Then Pd(dppf)Cl₂·DCM (79 mg, 0.096 mmol, 0.1 eq.) was added and degassed it for another 5 min. The reaction mixture was heated to 60° C. and stirred for 16 h. The reaction mixture was cooled to room temperature, filtered through the celite bed, diluted with water (20 mL) and extracted by EtOAc (3×30 mL). The combined organic phases were washed with brine (15 mL), dried over sodium sulphate and concentrated under reduced pressure to obtain the crude. The crude was purified by column C-18 Redisep reverse phase column chromatography to obtained 1-(5-(1-((1r,4R)-4-(2-((tert-butyldiphenylsilyl)oxy)ethyl)cyclohexyl)-1H-pyrazol-4-yl)-44(R)-1-cyanoethyl)amino)pyridin-2-yl)-1H pyrazolo[3,4-b]pyridine-5-carbonitrile 7′ (270 mg, 31.1% yield) as a colourless oil.

LCMS method 1: retention time: 1.752 min, 79.97% purity at 220 nm, [M+H]⁺=720.3.

Step 5′. Preparation of 1-(4-(((R)-1-cyanoethyl)amino)-5-(1-((1r,4R)-4-(2-hydroxyethyl)cyclohexyl)-1H-pyrazol-4-yl)pyridin-2-yl)-1H-pyrazolo[3,4-b]pyridine-5-carbonitrile A-80: To a stirred solution of 1-(5-(1-((1r,4R)-4-(2-((tert-butyldiphenylsilyl)oxy)ethyl)cyclohexyl)-1H-pyrazol-4-yl)-4-(((R)-1-cyanoethyl)amino)pyridin-2-yl)-1H-pyrazolo[3,4-b]pyridine-5-carbonitrile 7′ (230 mg, 0.319 mmol, 1.0 eq.) in THF (1 mL) was added hydrogen fluoride-pyridine (0.3 mL, 0.319 mmol, 1.0 eq.). The reaction mixture was warmed to room temperature and stirred for 3 h. The reaction mixture was treated with 10% sodium bicarbonate (10 mL) slowly and extracted with EtOAc (3×20 mL). The combined organic layer was washed with brine (20 mL), dried over sodium sulphate and concentrated under reduced pressure to give 1-(4-(((R)-1-cyanoethyl)amino)-5-(141r,4R)-4-(2-hydroxyethyl)cyclohexyl)-1H-pyrazol-4-yl)pyridin-2-yl)-1H-pyrazolo[3,4-b]pyridine-5-carbonitrile A-80 (145 mg, 40.5% yield) as a brown oil, which was used in the next step without further purification.

LCMS method 2: retention time: 1.951 min, [M+H]⁺=482.2.

TBM intermediates prepared using General Procedure TBM-13 are summarized in Table 26.

TABLE 26
TBM Intermediates Prepared via General Procedure TBM-13

| | Structure |
|---|---|
| A-80 | 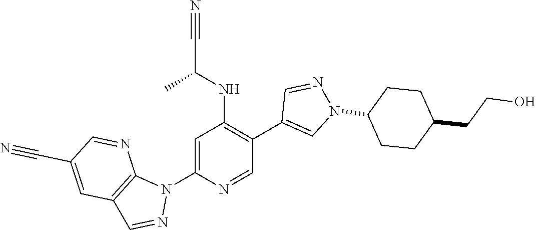 |

General Procedure TBM-14. The scheme shown below for the synthesis of A-81 is provided as a representative synthesis for General Procedure TBM-14.

### Example S37. 1-(4-(((R)-1-cyanoethyl)amino)-5-(3-((1r,4R)-4-(2-hydroxyethyl)cyclohexyl)isoxazol-5-yl)pyridin-2-yl)-1H-pyrazolo[3,4-b]pyridine-5-carbonitrile (A-81)

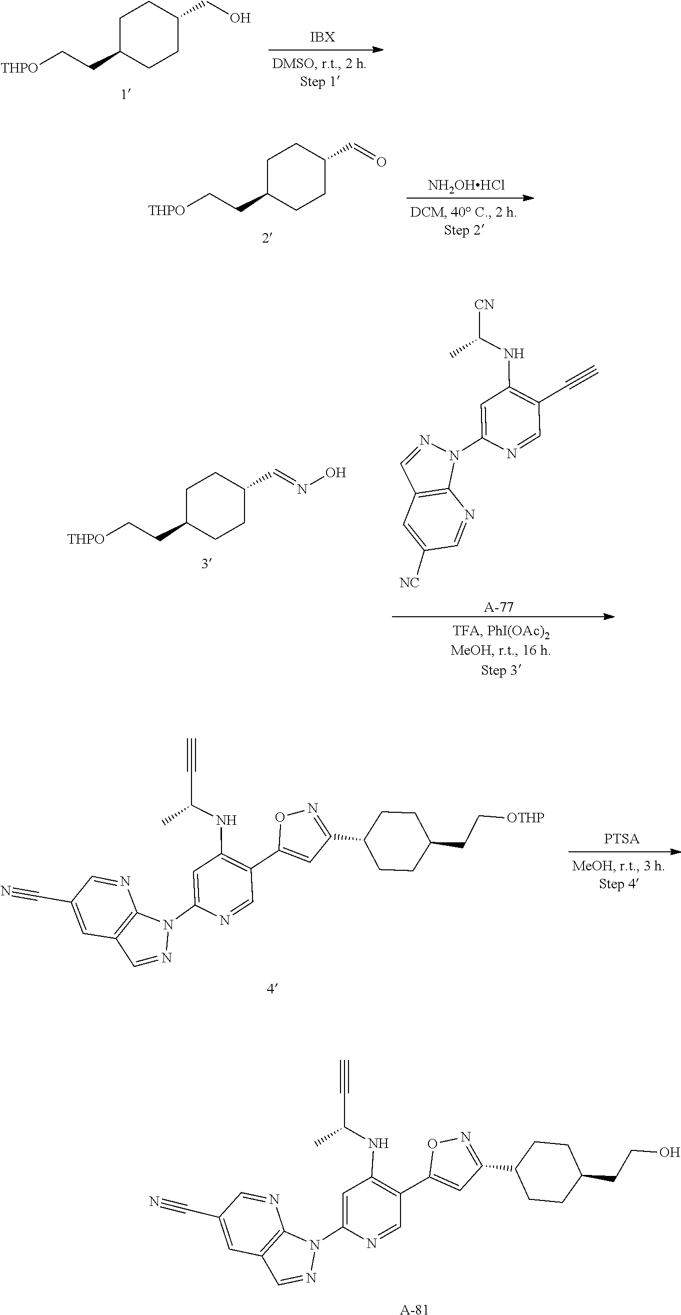

UPLC Method 1. Aquity UPLC BEH C-18, 50×3.0 mm, 1.7 Temperature: RT, Flow: 1.0 mL/min, Run Time: 5 minutes, Mobile Phase Conditions: Mobile Phase-A: 0.1% TFA in H₂O, Mobile Phase-B: 0.1% TFA in ACN, Gradient: Initial 80% Mobile Phase A and 20% Mobile Phase B linear gradient to 100% Mobile Phase B for 3.0 min. MSD positive.

LCMS Method 2. X-Bridge C-18, 50×4.6 mm, 5.0 μm, Temperature: RT, Flow: 1.0 mL/min, Run Time: 5 minutes, Mobile Phase Conditions: Mobile Phase-A: 0.1% TFA in H₂O, Mobile Phase-B: 0.1% TFA in ACN, Gradient: Initial 95% Mobile Phase A and 5% Mobile Phase B linear gradient to 5% Mobile phase A and 95% Mobile Phase B for 2.5 min. MSD positive.

Step 1′. Preparation of (1r,4r)-4-(2-((tetrahydro-2H-pyran-2-yl)oxy)ethyl)cyclohexane-1-carbaldehyde 2′: To a solution of ((1r,4r)-4-(2-((tetrahydro-2H-pyran-2-yl)oxy)ethyl)cyclohexyl)methanol 1′ (1.0 g, 4.13 mmol) in DMSO (2.0 mL) was added IBX (3.47 g, 12.38 mmol) at RT, under nitrogen atmosphere and the resulting solution was stirred for 2 h at RT. The reaction mixture was diluted with ice-cold water (10 mL) and extracted with ethyl acetate (3×20 mL). The combined organic layer was washed with aqueous sodium bicarbonate solution, followed by brine solution, dried over sodium sulphate, and concentrated under reduced pressure to give the crude product. The crude was purified by column chromatography using silica gel (100-200 mesh) with 0-30% ethyl acetate in pet ether to give a pure compound (1r,4r)-4-(2-((tetrahydro-2H-pyran-2-yl)oxy)ethyl)cyclohexane-1-carbaldehyde 2′ (900 mg, 91% yield) as a pale-yellow oil.

¹H NMR (400 MHz, DMSO-d6): δ ppm 0.94-1.04 (m, 2H), 1.15-1.18 (m, 1H), 1.25-1.52 (m, 8H), 1.55-1.83 (m, 4H), 1.85-1.97 (m, 2H), 2.18-2.25 (m, 1H), 3.32-3.44 (m, 2H), 3.65-3.72 (m, 2H), 4.53-4.54 (m, 1H), 9.55 (s, 1H).

Step 2′. Preparation of (E/Z)-4-(2-((tetrahydro-2H-pyran-2-yl)oxy)ethyl)cyclohexane-1-carbaldehyde oxime 3′: To a solution of (1r,4r)-4-(2-((tetrahydro-2H-pyran-2-yl)oxy)ethyl)cyclohexane-1-carbaldehyde 2′ (900 mg, 3.74 mmol) in DCM (5 mL) was added hydroxylamine hydrochloride (781 mg, 11.23 mmol) at RT. The resulting solution was stirred for 2 h at 40° C. The reaction mixture was cooled to RT and diluted with ice-cold water and extracted with DCM (3×20 mL). The combined organic layer was washed with brine, dried over sodium sulphate, and then concentrated under reduced pressure to give crude product. The crude product was purified by column chromatography using silica gel (100-200 mesh) with 30-50% ethyl acetate in pet ether to give compound (E/Z)-4-(2-((tetrahydro-2H-pyran-2-yl)oxy)ethyl)cyclohexane-1-carbaldehyde oxime 3′ (600 mg, 2.234 mmol, 59.7% yield) as a pale yellow liquid.

¹H NMR (400 MHz, DMSO-d6): δ ppm 0.93-1.04 (m, 2H), 1.08-1.22 (m, 2H), 1.25-1.52 (m, 7H), 1.54-1.80 (m, 6H), 2.01-2.11 (m, 1H), 3.34-3.48 (m, 2H), 3.62-3.80 (m, 2H), 4.52-4.54 (m, 1H), 6.43 (d, J=7.2 Hz, 0.3H), 7.21 (d, J=5.6 Hz, 0.7H), 10.35 (s, 10.65 (s, 0.3H).

Step 3′. Preparation of 1-(4-(((R)-1-cyanoethyl)amino)-5-(34(1r,4R)-4-(2-((tetrahydro-2H-pyran-2-yl)oxy)ethyl)cyclohexyl)isoxazol-5-yl)pyridin-2-yl)-1H-pyrazolo[3,4-b]pyridine-5-carbonitrile 4′: To a stirred solution of (E/Z)-4-(2-((tetrahydro-2H-pyran-2-yl)oxy)ethyl)cyclohexane-1-carbaldehyde oxime 3′(280 mg, 1.096 mmol) in MeOH (10 mL) was added (R)-1-(4-((l-cyanoethyl)amino)-5-ethynylpyridin-2-yl)-1H-pyrazolo[3,4-b]pyridine-5-carbonitrile A-77 (344 mg, 1.096 mmol), TFA (0.845 μl, 10.96 μmol) and PhI(OAc)₂ (389 mg, 1.206 mmol) at RT. The resulting solution was stirred for 16 h at RT. The reaction mixture was quenched with ice-cold ammonium chloride solution and extracted with ethyl acetate (3×20 mL). The combined organic layer was washed with brine, dried over sodium sulphate and then concentrated under reduced pressure to give crude product 1-(4-(((R)-1-cyanoethyl)amino)-5-(3-((1r,4R)-4-(2-((tetrahydro-2H-pyran-2-yl)oxy)ethyl)cyclohexyl)isoxazol-5-yl)pyridin-2-yl)-1H-pyrazolo[3,4-b]pyridine-5-carbonitrile 4′ (400 mg, 0.706 mmol), which was used in the next step without further purification.

LCMS method 1: retention time 2.65 min, [M+H]⁺=567.4.

Step 4′. Preparation of 1-(4-(((R)-1-cyanoethyl)amino)-5-(3-((1r,4R)-4-(2-hydroxyethyl)cyclohexyl)isoxazol-5-yl)pyridin-2-yl)-1H-pyrazolo[3,4-b]pyridine-5-carbonitrile A-81: To a stirred solution of 1-(4-(((R)-1-cyanoethyl)amino)-5-(3-((1r,4R)-4-(2-((tetrahydro-2H-pyran-2-yl)oxy)ethyl)cyclohexyl)isoxazol-5-yl)pyridin-2-yl)-1H-pyrazolo[3,4-b]pyridine-5-carbonitrile 4′ (400 mg, 0.706 mmol) in MeOH (5.0 mL) was added PTSA (135 mg, 0.706 mmol) at RT. The resulting solution was stirred for 3 h at RT. The reaction mixture was quenched with ice-cold sodium bicarbonate solution and extracted with ethyl acetate (3×20 mL). The combined organic layer was washed with brine, dried over sodium sulphate and then concentrated under reduced pressure to give the crude product 1-(4-(((R)-1-cyanoethyl)amino)-5-(3-((1r,4R)-4-(2-hydroxyethyl)cyclohexyl)isoxazol-5-yl)pyridin-2-yl)-1H-pyrazolo[3,4-b]pyridine-5-carbonitrile A-81 (200 mg, 26% yield), which was used in the next step without further purification.

LCMS method 2: retention time: 2.06 min, [M+H]⁺=483.3.

TBM intermediates prepared using General Procedure TBM-14 are summarized in Table 27.

TABLE 27
TBM Intermediates Prepared via General Procedure TBM-14

| | Structure |
|---|---|
| A-81 | 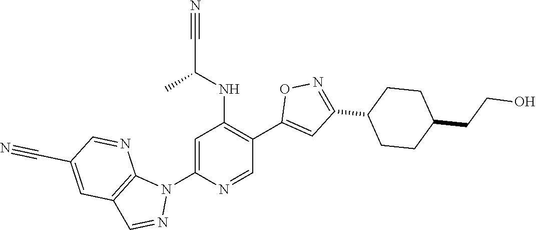 |

General Procedure TBM-15 for TBM Synthesis. The scheme shown below for the synthesis of A-84 is provided as a representative synthesis for General Procedure TBM-15.

### Example S38. 1-(4-(((R)-1-cyanoethyl)amino)-5-(1-((1r,4R)-4-(2-hydroxyethyl)cyclohexyl)-1H-1,2,4-triazol-3-yl)pyridin-2-yl)-1H-pyrazolo[3,4-b]pyridine-5-carbonitrile (A-84)

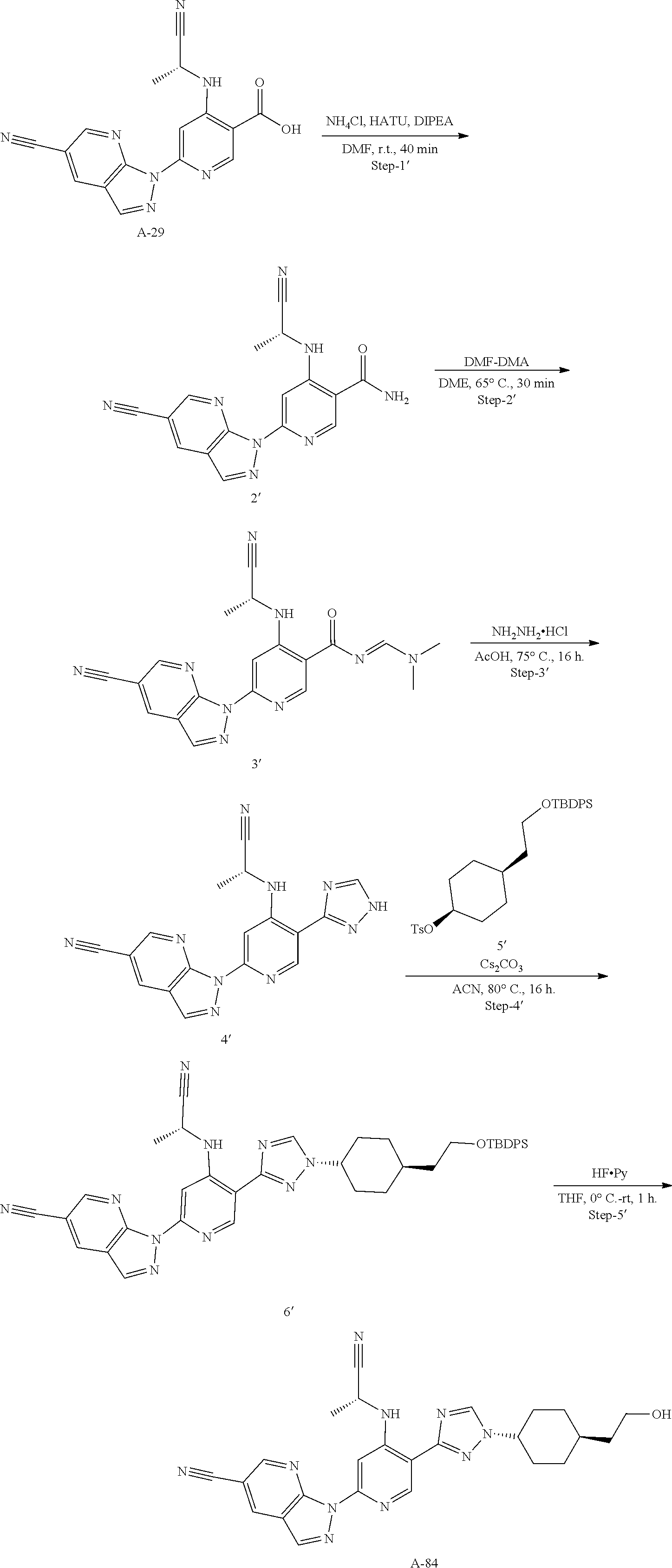

LCMS Method 1. Column: Kinetex XB-C18, 75×3.0 mm, 2.6 Temperature: RT, Flow: 1.0 mL/min, Run Time: 5 minutes, Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 100% Mobile Phase B for 4.0 min then hold for 1.6 min. MSD positive

UPLC Method 2. Column: Aquity BEH C18, 50×3.0 mm, 1.7 Temperature: RT, Flow: 0.7 mL/min, Run Time: 2.6 minutes, Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 98% Mobile Phase B for 1.5 min then hold for 0.5 min. MSD positive

Step 1′. Preparation of (R)-6-(5-cyano-1H-pyrazolo[3,4-b]pyridin-1-yl)-4-((1-cyanoethyl)amino)nicotinamide 2′: To a stirred solution of (R)-6-(5-cyano-1H-pyrazolo[3,4-b]pyridin-1-yl)-4-((1-cyanoethyl)amino)nicotinic acid A-29 (2.2 g, 4.29 mmol, 1.0 eq.) in N,N-Dimethylformamide (20.0 mL) was added ammonium chloride (0.459 g, 8.58 mmol, 2.0 eq.) and DIPEA (3.0 mL, 17.16 mmol, 4.0 eq.) and stirred it for 10 min. HATU (3.26 g, 8.58 mmol, 2.0 eq.) was added to the reaction mixture and stirred at RT for another 30 min. The reaction mixture was treated with water (10 mL) and extracted with DCM (2×30 mL). The combined organic layer was dried over sodium sulphate, filtered, and concentrated under reduced pressure to give crude (R)-6-(5-cyano-1H-pyrazolo[3,4-b]pyridin-1-yl)-4-((1-cyanoethyl)amino)nicotinamide 2′ (2.0 g), which was used in the next step without further purification.

LCMS method 1: retention time: 1.02 min, [M+H]⁺=333.0.

Step 2′. Preparation of (R,E)-6-(5-cyano-1H-pyrazolo[3,4-b]pyridin-1-yl)-4-((1-cyanoethyl)amino)-N((dimethylamino)methylene) nicotinamide 3′: At room temperature, (R)-6-(5-cyano-1H-pyrazolo[3,4-b]pyridin-1-yl)-4-((1-cyanoethyl)amino)nicotinamide 2′ (2.2 g, 3.64 mmol, 1.0 eq.) was dissolved in DME (2.0 mL) and added N,N-dimethylformamide dimethyl acetal (6.34 mL, 47.3 mmol, 13.0 eq.). The resulting mixture was heated to 65° C. and stirred for 30 min. The reaction mixture was cooled to RT and concentrated under reduced pressure to give (R,E)-6-(5-cyano-1H-pyrazolo[3,4-b]pyridin-1-yl)-4-(1-cyanoethyl)amino)-N-((dimethylamino)methylene)nicotinamide 3′ (2.0 g), which was used in the next step without further purification.

UPLC method 2: retention time: 0.818 min, [M−H]⁺=386.2.

Step 3′. Preparation of (R)-1-(4-((1-cyanoethyl)amino)-5-(1H-1,2,4-triazol-3-yl)pyridin-2-yl)-1H-pyrazolo[3,4-b]pyridine-5-carbonitrile 4′: (R,E)-6-(5-cyano-1H-pyrazolo[3,4-b]pyridin-1-yl)-4-((1-cyanoethyl)amino)-N-((dimethylamino)methylene) nicotinamide 3′ (2.0 g, 2.272 mmol, 1.0 eq.) was dissolved in acetic acid (2.0 mL) and added hydrazine monohydrochloride (0.311 g, 4.54 mmol, 2.0 eq.). The resulting mixture was heated at 75° C. and stirred for 16 h. The reaction mixture was cooled to RT and concentrated under reduced pressure to give product (R)-1-(4-((1-cyanoethyl)amino)-5-(1H-1,2,4-triazol-3-yl)pyridin-2-yl)-1H-pyrazolo[3,4-b]pyridine-5-carbonitrile 4′ (1.1 g), which was used in the next step without further purification.

LCMS method 1: retention time: 1.407 min, [M+H]⁺=357.2.

Step 4′. Preparation of 1-(5-(1-((1r,4R)-4-(2-((tert-butyldiphenylsilyl)oxy)ethyl)cyclohexyl)-1H-1,2,4-triazol-3-yl)-4-(((R)-1-cyanoethyl)amino)pyridin-2-yl)-1H-pyrazolo[3,4-b]pyridine-5-carbonitrile 6′: To a stirred solution of (R)-1-(4-((l-cyanoethyl)amino)-5-(1H-1,2,4-triazol-3-yl)pyridin-2-yl)-1H-pyrazolo[3,4-b]pyridine-5-carbonitrile 4′ (500 mg, 1.263 mmol, 1.0 eq.) in acetonitrile (5.0 mL) at RT was added (1s,4s)-4-(2-((tert-butyldiphenylsilyl)oxy)ethyl)cyclohexyl 4-methylbenzenesulfonate 5′ (678 mg, 1.263 mmol, 1.0 eq.) and cesium carbonate (1.234 g, 3.79 mmol, 3.0 eq.). The reaction mixture was heated to 80° C. and stirred for 16 h. The reaction mixture was cooled to RT, filtered through the celite and concentrated under reduced pressure to give crude product. The crude was purified by column chromatography using silica gel (230-400 mesh) with 50-60% EtOAc/pet ether to afford the 1-(5-(1-((1r,4R)-4-(2-((tertbutyldiphenylsilyl)oxy)ethyl)cyclohexyl)-1H-1,2,4-triazol-3-yl)-4-((R)-1-cyanoethyl)amino)pyridin-2-yl)-1H-pyrazolo[3,4-b]pyridine-5-carbonitrile 6′ (70 mg, 4.84% yield) as a colorless oil.

UPLC method 2: retention time: 2.11 min, 62.51% purity at 220 nm, [M+H]⁺=721.3.

Step 5′. Preparation of 1-(4-(((R)-1-cyanoethyl)amino)-5-(1-((1r,4R)-4-(2-hydroxyethyl)cyclohexyl)-1H-1,2,4-triazol-3-yl)pyridin-2-yl)-1H-pyrazolo[3,4-b]pyridine-5-carbonitrile A-84: To a stirred solution of 1-(5-(1-((1r,4R)-4-(2-((tertbutyldiphenylsilyl)oxy)ethyl)cyclohexyl)-1H-1,2,4-triazol-3-yl)-4-((R)-1-cyanoethyl)amino)pyridin-2-yl)-1H-pyrazolo[3,4-b]pyridine-5-carbonitrile 6′ (53 mg, 0.046 mmol, 1.0 eq.) in THF (1.0 mL) at 0° C. was added hydrogen fluoride-pyridine (0.0093 mL, mmol, 1.0 eq. 70% hydrogen fluoride basis) and stirred the reaction mixture at RT for 1 h. The reaction mixture was quenched by adding 10% sodium bicarbonate (5.0 mL) slowly and extracted with EtOAc (3×20 mL). The organic layer was dried over sodium sulphate, filtered and concentrated under reduced pressure to give 1-(4-(((R)-1-cyanoethyl)amino)-5-(141r,4R)-4-(2-hydroxyethyl)cyclohexyl)-1H-1,2,4-triazol-3-yl)pyridin-2-yl)-1H-pyrazolo[3,4-b]pyridine-A-84 (70 mg) as a brown oil, which was used in the next step without further purification.

LCMS method 1: retention time: 2.013 min, [M+H]⁺=483.2.

TBM intermediates prepared using General Procedure TBM-15 are summarized in Table 28.

TABLE 28
TBM Intermediates Prepared via General Procedure TBM-15

| | Structure |
|---|---|
| A-84 | 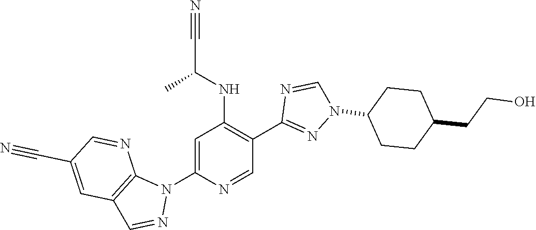 |

General Procedure TBM-16. The scheme shown below for the synthesis of A-85 is provided as a representative synthesis for General Procedure TBM-16.

### Example S39. 1-(4-(((R)-1-cyanoethyl)amino)-5-(4-(4-hydroxy-4-(2-hydroxyethyl)cyclohexyl)-1H-1,2,3-triazol-1-yl)pyridin-2-yl)-1H-pyrazolo[3,4-b]pyridine-5-carbonitrile (A-85)

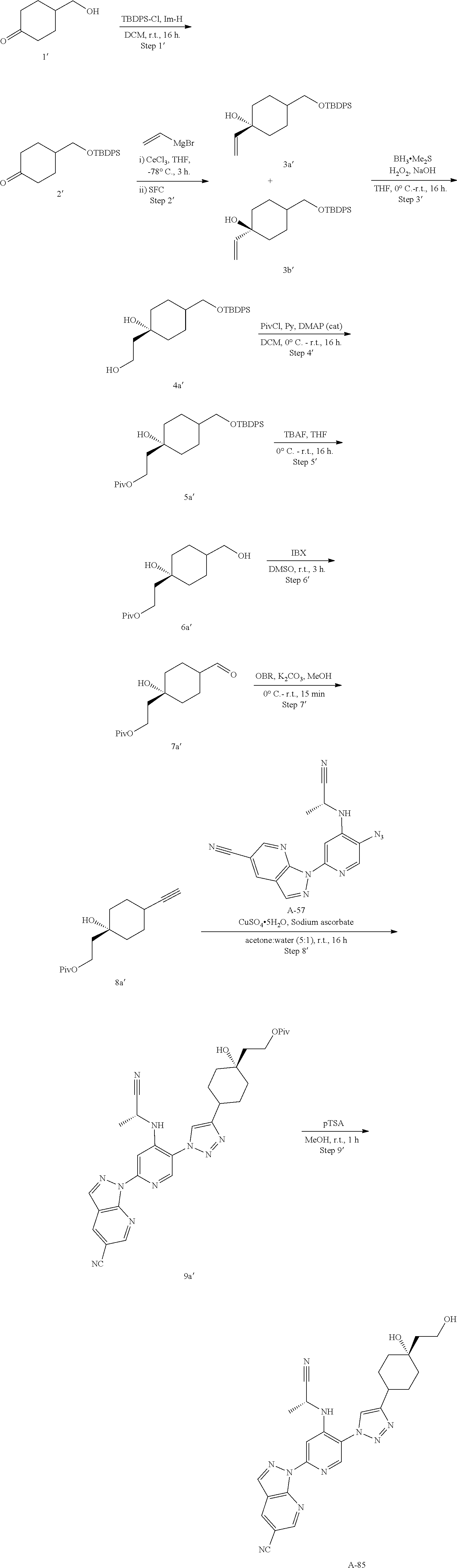

UPLC Method 1. Aquity BEH C18, 50×3.0 mm, 1.7 Temperature: RT, Flow: 0.7 mL/min, Run Time: 2.5 minutes, Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 98% Mobile Phase B for 1.5 min. MSD positive.

Step 1′. Preparation of 4-(((tert-butyldiphenylsilyl)oxy)methyl)cyclohexan-1-one 2′: To a solution of 4-(hydroxymethyl)cyclohexan-1-one 1′ (5.0 g, 39.0 mmol, 1.0 eq.) and 1H-imidazole (5.31 g, 78 mmol, 2.0 eq.) in DCM (50 mL, 0.7 M) at 0° C. was added TBDPS-C₁ (12.87 g, 46.8 mmol, 1.2 eq.). The reaction was stirred at RT for 16 h. The reaction mixture was quenched with water (50 mL) and extracted with DCM (3×50 mL). The organic layer was washed with brine (15 mL), dried over sodium sulphate, filtered, and concentrated under reduced pressure to obtain the crude. The crude was purified by silica gel chromatography with EtOAc/pet ether to afford 4-(((tert-butyldiphenylsilyl)oxy)methyl)cyclohexan-1-one 2′ (11.0 g, 77% yield) as a white semi-solid.

¹H NMR (400 MHz, CDCl₃): δ ppm 1.09 (s, 9H), 1.51-1.57 (m, 2H), 1.94-2.18 (m, 3H), 2.34-2.41 (m, 4H), 3.59 (d, J=6.4 Hz, 2H), 7.38-7.46 (m, 6H), 7.67-7.75 (m, 4H)

Step 2′. Preparation of (1s,4s)-4-(((tert-butyldiphenylsilyl)oxy)methyl)-1-vinylcyclohexan-1-ol 3a′ and (1r,4r)-4-(((tert-butyldiphenylsilyl)oxy)methyl)-1-vinylcyclohexan-1-ol 3b′: To a solution of (1s,4s)-4-(((tert-butyldiphenylsilyl)oxy)methyl)-1-vinylcyclohexan-1-ol 2′ (7.0 g, 19.10 mmol, 1.0 eq.) in THF (50 mL) under nitrogen atmosphere at −78° C. was added vinyl magnesium bromide (24.0 mL, 24.82 mmol, 1.3 eq., 1.0 M in THF) dropwise (over 20 min). The reaction was stirred at −78° C. for 2 h. The reaction mixture was quenched with saturated ammonium chloride solution (15 mL) and warmed up to RT. The aqueous phase was extracted with EtOAc (3×80 mL). The combined organic phases were washed once with brine (30 mL), dried over sodium sulphate, filtered, and concentrated under reduced pressure. The residue was purified by flash column chromatography on silica with 15-20% EtOAc/pet ether to afford the minor product (1s,4s)-4-(((tert-butyldiphenylsilyl)oxy)methyl)-1-vinylcyclohexan-1-ol 3a′ (0.8 g, 10.62% yield) and major product (1r,4r)-4-(((tert-butyldiphenylsilyl)oxy)methyl)-1-vinylcyclohexan-1-ol 3b′ (2.1 g, 27.9% yield) as a brown thick liquid.

Characterization of Compound 3a′: ¹H NMR (400 MHz, CDCl₃): δ ppm 1.07 (s, 9H), 1.37-1.56 (m, 4H), 1.64-1.72 (m, 5H), 3.53 (d, J=6.0 Hz, 2H), 5.04 (dd, J=1.2 Hz and 10.8 Hz, 1H), 5.26 (dd, J=1.2 Hz and 17.6 Hz, 1H), 5.97 (dd, J=10.8 Hz and 17.2 Hz, 1H), 7.38-7.44 (m, 6H), 7.68-7.70 (m, 4H).

Characterization of Compound 3b′: ¹H NMR (400 MHz, CDCl₃): δ ppm 1.07 (s, 9H), 1. 19-1.28 (m, 3H), 1.42-1.69 (m, 2H), 1.76-1.85 (m, 4H), 3.59 (d, J=6.4 Hz, 2H), 5.16 (dd, J=1.2 Hz and 10.8 Hz, 1H), 5.32 (dd, J=1.2 Hz and 17.6 Hz, 1H), 6.07 (dd, J=11.2 Hz and 17.6 Hz, 1H), 7.38-7.46 (m, 6H), 7.66-7.69 (m, 4H).

Step 3′. Preparation of (1s,4s)-4-(((tert-butyldiphenylsilyl)oxy)methyl)-1-(2-hydroxyethyl)cyclohexan-1-ol 4a′: To a solution of (1s,45)-4-(((tert-butyldiphenylsilyl)oxy)methyl)-1-vinylcyclohexan-1-ol 3a′ (700 mg, 1.774 mmol, 1.0 eq.) in THF (5 mL, 0.35 M) at 0° C. was added Borane dimethyl sulfide complex (0.7 mL, 4.43 mmol, 2.5 eq.) dropwise (over 10 min). The resulting mixture was warmed to room temperature and stirred for 4 h. Then the reaction mass was cooled to 0° C. and added NaOH (0.7 mL, 1.774 mmol, 1.0 eq.) followed by hydrogen peroxide (0.7 mL, 22.84 mmol, 1.0 eq.) in a dropwise manner. The reaction was stirred at RT for another 16 h. The reaction mixture was quenched with ice-cold water (20 mL) and extracted with EtOAc (3×30 mL). The combined organic phases were washed with brine (20 mL), dried over sodium sulphate, filtered and concentrated under reduced pressure. The residue was purified by flash column chromatography on silica with 15-20% EtOAc/pet ether to afford (1s,4s)-4-(((tert-butyldiphenylsilyl)oxy)methyl)-1-(2-hydroxyethyl)cyclohexan-1-ol 4a′ (550 mg, 75% yield) as a thick brown liquid.

¹H NMR (400 MHz, CDCl₃): δ ppm 0.88-0.93 (m, 1H), 1.07 (s, 9H), 1.31-1.87 (m, 10H), 3.52 (d, J=6.0 Hz, 2H), 4.06 (t, J=5.6 Hz, 2H), 7.38-7.46 (m, 6H), 7.67-7.70 (m, 4H).

Step 4′. Preparation of 24(1s,4s)-4-(((tert-butyldiphenylsilyl)oxy)methyl)-1-hydroxycyclohexyl)ethyl pivalate 5a′: To a solution of (1s,4s)-4-(((tert-butyldiphenylsilyl)oxy)methyl)-1-(2-hydroxyethyl)cyclohexan-1-ol 4a′ (450 mg, 1.091 mmol, 1.0 eq.) in DCM (3 mL, 0.364 M) under nitrogen was added pivaloyl chloride (197 mg, 1.636 mmol, 1.5 eq.) followed by pyridine (259 mg, 3.27 mmol, 3.0 eq.) and 4-dimethylaminopyridine (6.66 mg, 0.055 mmol, 0.05 eq.). The reaction was stirred at RT for 16 h. The reaction mixture was quenched with water (20 mL), and the aqueous phase was extracted with EtOAc (3×30 mL). The combined organic phases were washed with brine (20 mL), dried over sodium sulphate, filtered and concentrated under reduced pressure to give crude 1-(2-((1s,4s)-4-(((tert-butyldiphenylsilyl)oxy)methyl)-1-hydroxycyclohexyl)ethoxy)-3,3-dimethylbutan-2-one 5a′ (510 mg), which was used in the next step without further purification.

Step 5′. Preparation of 2-((1s,4s)-1-hydroxy-4-(hydroxymethyl)cyclohexyl)ethyl pivalate 6a′: To a solution of 1-(2-((1s,4s)-4-(((tert-butyldiphenylsilyl)oxy)methyl)-1-hydroxycyclohexyl)ethoxy)-3,3-dimethylbutan-2-one 5a′ (1.1 g, 2.214 mmol, 1.0 eq.) in THF (3.0 mL, 0.738 M) was added TBAF (1.67 mL, 3.32 mmol, 1.5 eq., 2.0 M in THF) dropwise at room temperature. The reaction was stirred at RT for 16 h. The reaction mixture was quenched with ice-cold water (30 mL) and extracted with EtOAc (3×30 mL). The combined organic phases were washed with brine (20 mL), dried over sodium sulphate, filtered and concentrated under reduced pressure to give the crude product. The residue was purified by flash column chromatography on silica with 20-25% EtOAc/pet ether to afford 1-(2-((1s,4s)-1-hydroxy-4-(hydroxymethyl)cyclohexyl)ethoxy)-3,3-dimethylbu-tan-2-one 6a′ (180 mg, 84% yield) as a yellow liquid.

¹H NMR (400 MHz, CDCl₃): δ ppm 1.11-1.19 (m, 2H), 1.20 (s, 9H), 1. 41-1.49 (m, 3H), 1.76-1.82 (m, 4H), 1.89 (t, J=6.4 Hz, 2H), 3.51 (d, J=6.0 Hz, 2H), 4.25 (t, J=6.4 Hz, 2H).

Step 6′. Preparation of 2-((1s,4s)-4-formyl-1-hydroxycyclohexyl)ethyl pivalate 7a′: To a solution of 2-((1r,4r)-1-hydroxy-4-(hydroxymethyl)cyclohexyl)ethyl pivalate 6a′ (430 mg, 1.664 mmol, 1.0 eq.) in DMSO (2 mL, 0.832 M) under nitrogen atmosphere was added IBX (1.39 g, 4.99 mmol, 3.0 eq.) and stirred at RT for 2 h. The reaction mixture was treated with ice-cold water (30 mL) and extracted with EtOAc (3×30 mL). The combined organic phases were washed with brine (20 mL), dried over sodium sulphate, filtered and concentrated under reduced pressure to give the crude product 2-((1r,4r)-4-formyl-1-hydroxycyclohexyl)ethyl pivalate 7a′ (410 mg) as a thick brown liquid, which was used in the next step without further purification.

¹H NMR (400 MHz, CDCl₃): δ ppm 1.21 (s, 9H), 1. 56-1.63 (m, 4H), 1.72-1.78 (m, 2H), 1.83 (t, J=6.4 Hz, 2H), 1. 96-2.06 (m, 2H), 2.36-2.42 (m, 1H), 4.26 (t, J=6.4 Hz, 2H), 9.71 (s, 1H).

Step 7′. Preparation of 2-((1s,4s)-4-ethynyl-1-hydroxycyclohexyl)ethyl pivalate 8a′: To a solution of 2-((1r,4r)-4-formyl-1-hydroxycyclohexyl)ethyl pivalate 7a′ (400 mg, 1.560 mmol, 1.0 eq) in MeOH (2.0 mL, 0.780 M) under nitrogen atmosphere was added Ohira-Bestmann reagent (360 mg, 1.872 mmol, 1.2 eq) followed by potassium carbonate (647 mg, 4.68 mmol, 3.0 eq) and the reaction mixture was stirred at RT for 15 min. The reaction mixture was concentrated under reduced pressure, treated with ice-cold water (20 mL) and extracted with EtOAc (3×30 mL). The combined organic phases were washed with brine (20 mL), dried over sodium sulphate, filtered and concentrated under reduced pressure to obtain the crude product 2-((1r,4r)-4-ethynyl-1-hydroxycyclohexyl)ethyl pivalate 8a′ (130 mg, 70% yield) as a green liquid, which was used in the next step without further purification.

¹H NMR (400 MHz, CDCl₃): δ ppm 1.21 (s, 9H), 1.39-1.42 (m, 1H), 1.73-1.93 (m, 9H), 2.07 (d, J=2.4 Hz, 1H), 2.26-2.34 (m, 1H), 4.25-4.31 (m, 2H).

Step 8′. Preparation of 2-(4-(1-(6-(5-cyano-1H-pyrazolo[3,4-b]pyridin-1-yl)-4-(((R)-1cyanoethyl)amino)pyridin-3-yl)-1H-1,2,3-triazol-4-yl)-1-hydroxycyclohexyl)ethyl pivalate 9a′: To a stirred solution of (R)-1-(5-azido-4-((1-cyanoethyl)amino)pyridin-2-yl)-1H-pyrazolo[3,4-b]pyridine-5-carbonitrile A-57 (315 mg, 0.668 mmol, 1.0 eq.) and 2-((1r,4r)-4-ethynyl-1-hydroxycyclohexyl)ethyl pivalate 8a′ (101 mg, 0.401 mmol, 0.6 eq) in acetone (2.4 mL) was added sodium ascorbate (66.1 mg, 0.334 mmol, 0.5 eq) and copper(II) sulphate pentahydrate (83 mg, 0.334 mmol, 0.5 eq) in water (0.6 mL). The reaction mixture was stirred at RT for 2 h, treated with water (10.0 mL) and the aqueous phase was extracted with EtOAc (3×30 mL). The combined organic phases were washed with brine (20 mL), dried over sodium sulphate, filtered and concentrated under reduced pressure to obtain the crude product 2-(4-(1-(6-(5-cyano-1H-pyrazolo[3,4-b]pyridin-1-yl)-4-((R)-1-cyanoethyl) amino)pyridin-3-yl)-1H-1,2,3-triazol-4-yl)-1-hydroxycyclohexyl)ethyl pivalate 9a′ (280 mg) as a brown liquid, which was used in the next step without further purification.

LCMS method 1: retention time: 1.31 min, 81.59% purity at 220 nm, [M+H]⁺=583.2.

Step 9′. Preparation of 1-(4-(((R)-1-cyanoethyl)amino)-5-(4-(4-hydroxy-4-(2-hydroxyethyl)cyclohexyl)-1H-1,2,3-triazol-1-yl)pyridin-2-yl)-1H-pyrazolo[3,4-b]pyridine-5-carbonitrile A-85: To a solution of 2-(4-(1-(6-(5-cyano-1H-pyrazolo[3,4-b]pyridin-1-yl)-4-(((R)-1-cyanoethyl)amino)pyridin-3-yl)-1H-1,2,3-triazol-4-yl)-1-hydroxycyclohexyl)ethyl pivalate 9a′ (100 mg, 0.172 mmol, 1.0 eq.) in MeOH (3.0 mL, 0.057 M) under nitrogen atmosphere was added p-toluenesulfonic acid monohydrate (98 mg, 0.515 mmol, 3.0 eq.) The reaction mixture was stirred at RT for 2 h, treated with water (10.0 mL) and the aqueous phase was extracted with EtOAc (3×20 mL). The combined organic phases were washed with brine (20 mL), dried over sodium sulphate, filtered and concentrated under reduced pressure to obtain the crude product 1-(4-(((R)-1-cyanoethyl)amino)-5-(4-((1s,4S)-4-hydroxy-4-(2-hydroxyethyl)cyclohexyl)-1H-1,2,3-triazol-1-yl)pyridin-2-yl)-1H-pyrazolo[3,4-b]pyridine-5-carbonitrile A-85 (65 mg) as a brown thick liquid, which was used in the next step without further purification.

LCMS method 1: retention time: 0.863 min, [M+H]⁺=499.2.

TBM intermediates prepared using General Procedure TBM-16 are summarized in Table 29.

TABLE 29
TBM Intermediates Prepared via General Procedure TBM-16

| | Structure |
|---|---|
| A-85 | 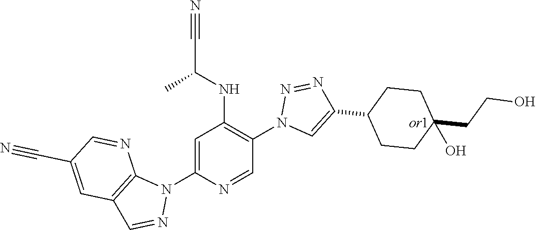 |

Synthesis of the Final Compound with the CBM and TBM Molecules

The numbering of the intermediate compounds referred to in this section is limited to each section only. For instance, intermediate 3 in General Procedure X-1 and intermediate 3 in General Procedure X-2 are not the same compounds as they are from different sections.

### Final Product LCMS Methods

LCMS Method numbers are limited to this section only.

LCMS Method 1: Column: Luna C18 (2) 50×3 mm, 3 um. Temperature: 45° C., Flow: 1.5 mL/min, run time: 2.5 min. Mobile phase conditions: Initial 95% H₂O 0.1% FA/% MeCN 0.1% FA, linear gradient to 95 MeCN 0.1% FA over 1.3 min then hold for 1.2 minute at 95 MeCN 0.1% FA. MSD: ESI Positive.

LCMS Method 2: Column: SunFire C18 75×4.6 mm, 3.5 um. Temperature: 45° C., Flow: 1.5 mL/min, run time: 6 min. Mobile phase conditions: Initial 95% H₂O+0.1% FA/% MeCN+0.1% FA then linear gradient to 95% MeCN+0.1% FA for 4 min then hold for 2 min at 95 MeCN+0.1% FA. MSD: Positive.

LCMS Method 3: Column: Luna C18 (2) 50×3 mm, 3 um. Temperature: 45° C., Flow: 1.5 mL/min, run time: 3.5 min. Mobile phase conditions: Initial 95% H₂O 0.1% FA/5% MeCN 0.1% FA, linear gradient to 95 MeCN 0.1% FA over 1.3 min then hold for 2.2 minute at 95 MeCN 0.1% FA. MSD: ESI Positive.

LCMS Method 4: C18 4.6×100 mm, Initial Gradient at 95% H₂O+0.1% FA/5% MeCN+0.1% FA 6 min run with 1. 5 min equilibration Gradient 0 to 4 min at 95% H₂O to 0% and hold for 2 minute.

LCMS Method 5: Kinetex Polar C18 2.6 um, 50×3.0 mm. Temperature: 40 C, Flow: 1.2 mL/min, Run time: 6 min. Mobile phase conditions: Initial 95% H₂O+0.1% FA/5% CH₃CN+0.1% FA then linear gradient to 95% CH₃CN for 3.5 min then hold for 2.5 min at 95% CH₃CN. MSD: Positive.

LCMS Method 6: XBridge C18 4.6×75 mm Sum, Initial Gradient at 95% NH₄HCO3/5% MeCN 6 min run with 1 min equilibration Gradient 0 to 3 min at 95% MeCN and hold for 3 minutes. Flow 1.5 mL/min.

LCMS Method 7: Kinetex XB-C18, 75×3.0 mm, 2.6 Temperature: RT, Flow: 1.0 mL/min, Run Time: 5 minutes, Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 100% Mobile Phase B for 4.0 min. MSD positive.

LCMS Method 8: X-Bridge C8, 50×4.6 mm, 3.5 Temperature: RT, Flow: 1.5 mL/min, Run Time: 6 minutes, Mobile Phase Conditions: Mobile Phase-A: 0.1% in TFA in H₂O, Mobile Phase-B: 0.1% in TFA in acetonitrile, Gradient: Initial 95% Mobile Phase A and 5% Mobile Phase B linear gradient to 5% Mobile phase A and 95% Mobile Phase B for 2.5 min. MSD positive.

LCMS Method 9: SunFire C18 4.6×75 mm, Initial Gradient at 95% H₂O+0.1% FA /5% MeCN+0.1% FA 10 min run with 1.5 min equilibration Gradient 0 to 8 min at 95% H₂O to 0% and hold for 2 minute.

LCMS Method 10: XSELECT C-18, 50×4.6 mm, 5.0 μm, Temperature: RT, Flow: 1.5 mL/min, Run Time: 5 minutes, Mobile Phase Conditions: Mobile Phase-A: 10 mm Ammonium Acetate in H₂O, Mobile Phase-B: 100% ACN, Gradient: Initial 95% Mobile Phase A and 5% Mobile Phase B linear gradient to 5% Mobile phase A and 95% Mobile Phase B for 2.5 min. MSD positive.

LCMS Method 11: Kinetex XB-C18, 50×4.6 mm, 5 μm, Temperature: RT, Flow: 1.0 mL/min, Run Time: 6 min, Mobile Phase Conditions: Mobile phase-A: 0.1% TFA in H₂O, Mobile phase-B: 0.1% TFA in CH₃CN, Gradient: Initial 5% Mobile Phase A and 95% Mobile Phase B linear gradient to 100% Mobile Phase B for 2.5 min. MSD positive.

LCMS Method 12: X-SELECT, 150×4.6 mm, 3.5 Temperature: RT, Flow: 1.0 mL/min, Run Time: 25 minutes, Mobile Phase Conditions: Mobile Phase-A: 5.0 mm Ammonium formate Buffer:ACN (98:2), Mobile Phase-B: 5.0 mm ACN:buffer(98:2), Gradient: Initial 90% Mobile Phase A and 10% Mobile Phase B linear gradient to 100% Mobile Phase B for 18.0 min. MSD positive.

LCMS Method 13: LCMS method: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+/−).

### General Procedure X-1

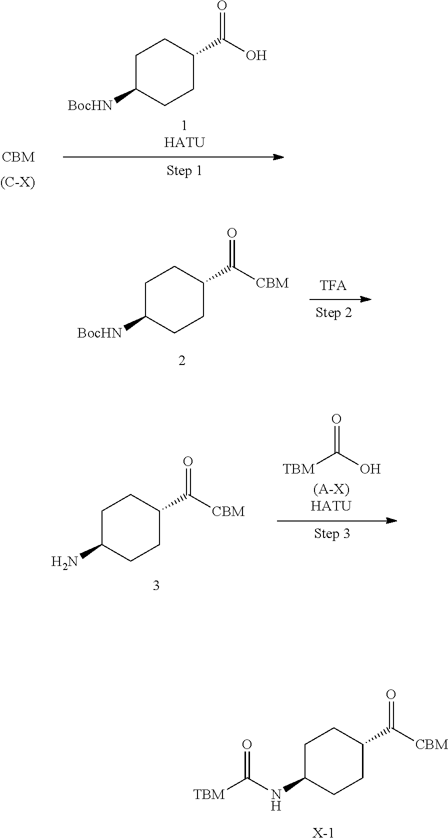

Step 1. Preparation of 2 via amide coupling of CBM amine (C-X) to 4-((tert-butoxycarbonyl)amino)cyclohexane-1-carboxylic acid: To a solution of 4-(tert-butoxycarbonylamino)cyclohexanecarboxylic acid 1 (1 eq.) and CBM amine (C-X) (1.2 eq.) in DMF (0.4 M) was added DIPEA (27.8 eq.). After 5 minutes of stirring at room temperature, HATU (1.3 eq.) was added. The solution was then stirred at room temperature. After LCMS showed complete conversion, the reaction was stopped and purified to afford 2.

Step 2. Preparation of 3 via Boc-deprotection of 2 with trifluoroacetic acid: To a solution of 2 (1 eq.) in DCM (0.94 M) was added TFA (31 eq.). The reaction mixture was stirred at room temperature. After LCMS showed full conversion into compound 3. The reaction mixture was concentrated under reduced pressure and the residue purified to give 3 as a trifluoroacetic acid salt.

Step 3. Preparation of final product via amide coupling of 3 and TBM carboxylic acid (A-X): To a solution of TBM carboxylic acid (A-X) (1 eq.) and amine TFA salt 3 (1.5 eq.) in DMF (0.2 M) was added DIPEA (5 eq.). After 5 minutes of stirring at room temperature, HATU (1.1 eq.) was added. The solution was then stirred at room temperature. After LCMS showed complete conversion into final product, the reaction was stopped and purified to afford final product X-1.

### Example S40. 6-(1,3-benzothiazol-6-ylamino)-N-[4-[4-[2-(2,6-dioxo-3-piperidyl)-1,3-dioxo-isoindolin-5-yl]piperazine-1-carbonyl]cyclohexyl]-4-[(1-methylsulfonyl-4-piperidyl)amino]pyridine-3-carboxamide (P-4)

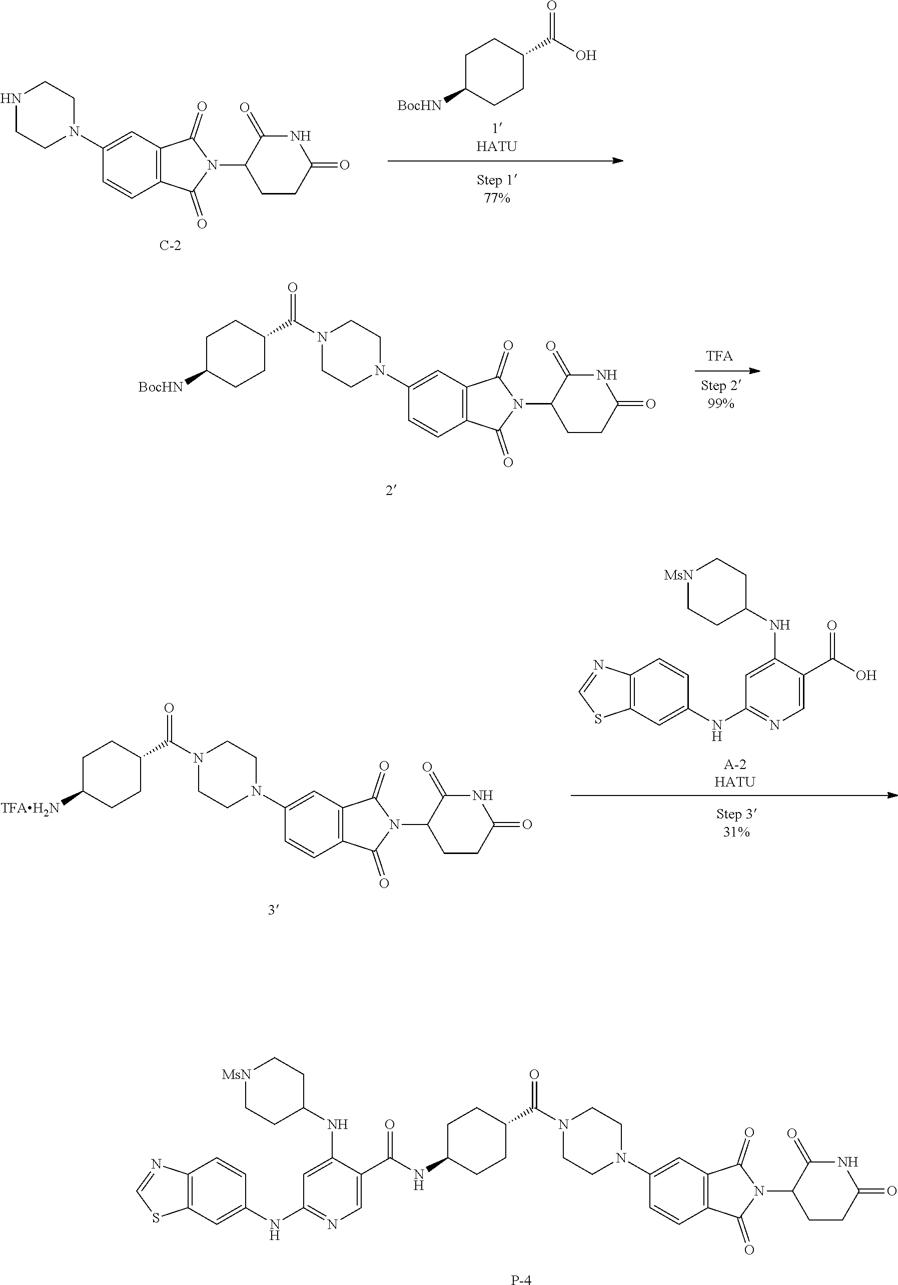

Step 1′. Preparation of tert-Butyl N-[4-[4-[2-(2,6-dioxo-3-piperidyl)-1,3-dioxo-isoindolin-5-yl]piperazine-1-carbonyl]cyclohexyl]carbamate (2′): To a solution of 4-(tert-butoxycarbonylamino)cyclohexanecarboxylic acid 1′ (502 mg, 2.06 mmol, 1 eq.) and 2-(2,6-dioxo-3-piperidyl)-5-piperazin-1-yl-isoindoline-1,3-dione C-2 (1.13 g, 2.48 mmol, 1.2 eq.) in DMF (5 mL, 0.4 M) was added DIPEA (10 mL, 57.41 mmol, 27.8 eq.). After 5 minutes of stirring at room temperature, HATU (1.03 g, 2.72 mmol, 1.3 eq.) was added. The solution was then stirred at room temperature. After 30 min., LCMS showed complete conversion into compound 9. The reaction was stopped and it was directly loaded for reverse phase FC purification (150 g C18 gold column, liquid deposit (reaction mixture), 5% MeOH/0.1% HCOOH over 4 CV, then 5 to 95% MeOH/0.1% HCOOH over 10 CV), affording the desired product albeit contaminated. The contaminated product was then purified by normal phase flash chromatography (80 g column, solid deposit, elution 0% to 10% MeOH/DCM over 10 CV). Fractions were combined and concentrated, affording 2′ (938.5 g, 77% yield) as a light-yellow solid.

LCMS method 1: 99.9% purity at 215 nm [M+H]⁺=468.2.

¹H NMR (400 MHz, chloroform-d) δ ppm 1.08-1.21 (m, 2H), 1.45 (s, 9H), 1.64-1.77 (m, 2H), 1.78-1.86 (m, 2H), 2.06-2.21 (m, 3H), 2.40-2.50 (m, 1H), 2.68-2.94 (m, 3H), 3.40-3.48 (m, 4H), 3.70 (br s, 2H), 3.80 (br s, 2H), 4.41 (br s, 1H), 4.95 (dd, J=12.2, 5.4 Hz, 1H), 7.07 (dd, J=8.6, 2.2 Hz, 1H), 7.29 (d, J=2.0 Hz, 1H), 7.73 (d, J=8.3 Hz, 1H), 8.16 (s, 1H). One proton was missing.

Step 2′. Preparation of 5-[4-(4-aminocyclohexanecarbonyl)piperazin-1-yl]-2-(2,6-dioxo-3-piperidyl)isoindoline-1,3-dione;2,2,2-trifluoroacetic acid (3′): To a solution of tert-butyl N-[4-[4-[2-(2,6-dioxo-3-piperidyl)-1,3-dioxo-isoindolin-5-yl]piperazine-1-carbonyl]cyclohexyl]carbamate 2′ (938.5 mg, 1.65 mmol, 1 eq.) in DCM (1.8 mL, 0.94 M) was added TFA (4 mL, 51.56 mmol, 31 eq.). The reaction mixture was stirred at room temperature. After an overnight period, LCMS showed full conversion into compound 3′. The reaction mixture was concentrated under reduced pressure and the residue was co-evaporated with toluene (2×) and MeCN (2×) to give 3′ (962 mg, 99% yield) as a yellow solid as a trifluoroacetic acid salt.

LCMS method 1: 98.9% purity at 215 nm, [M-CF₃COOH+H]⁺=468.6.

¹H NMR (400 MHz, DMSO-d6) δ ppm 1.31-1.52 (m, 4H), 1.76 (br d, J=12.5 Hz, 2H), 1.90-1.98 (m, 2H), 1.98-2.06 (m, 1H), 2.52-2.68 (m, 3H), 2.82-2.94 (m, 1H), 2.95-3.06 (m, 1H), 3.41-3.55 (m, 4H), 3.57-3.72 (m, 1H), 3.68 (br s, 1H), 5.08 (dd, J=13.3, 5.3 Hz, 1H), 7.22-7.24 (m, 1H), 7.25-7.28 (m, 1H), 7.35 (d, J=2.0 Hz, 1H), 7.71 (d, J=8.6 Hz, 1H), 7.76-7.86 (m, 3H), 11.08 (s, 1H).

Step 3′. Preparation of 6-(1,3-benzothiazol-6-ylamino)-N-[4-[4-[2-(2,6-dioxo-3-piperidyl)-1,3-dioxo-isoindolin-5-yl]piperazine-1-carbonyl]cyclohexyl]-4-[(1-methylsulfonyl-4-piperidyl)amino]pyridine-3-carboxamide (P-4): To a solution of 6-(1,3-benzothiazol-6-ylamino)-4-[(1-methylsulfonyl-4-piperidyl)amino]pyridine-3-carboxylic acid A-2 (50 mg, 0.11 mmol, 1 eq.) and 5-[4-(4-aminocyclohexanecarbonyl)piperazin-1-yl]-2-(2,6-dioxo-3-piperidyl)isoindoline-1,3-dione;2,2,2-trifluoroacetic acid 3′ (97.46 mg, 0.17 mmol, 1.5 eq.) in DMF (0.56 mL, 0.2 M) was added DIPEA (97.3 0.56 mmol, 5 eq.). After 5 minutes of stirring at room temperature, HATU (46.73 mg, 0.12 mmol, 1.1 eq.) was added. The solution was then stirred at room temperature. After 1 h, LCMS showed complete conversion into compound ATN-107-284404. The reaction was stopped and it was directly loaded for reverse phase FC purification (50 g C18 column, liquid deposit (reaction mixture), 5% MeCN/0.1% HCOOH over 4 CV, then 5 to 70% MeCN/0.1% HCOOH over 12 CV). Fractions were combined and concentrated to give P-4 (30 mg, 31% yield) as a yellow solid.

LCMS method 2: 99.9% purity at 215 nm [M+H]⁺=897.1.

¹H NMR (400 MHz, DMSO-d6) δ ppm 1.34-1.61 (m, 6H), 1.71-1.81 (m, 2H), 1.84-1.96 (m, 2H), 1.97-2.10 (m, 3H), 2.52-2.69 (m, 3H), 2.81-3.02 (m, 6H), 3.40-3.57 (m, 7H), 3.57-3.65 (m, 2H), 3.65-3.75 (m, 3H), 5.03-5.14 (m, 1H), 6.09 (s, 1H), 7.26 (d, J=8.3 Hz, 1H), 7.35 (s, 1H), 7.53 (d, J=8.6 Hz, 1H), 7.71 (d, J=8.6 Hz, 1H), 7.93-8.05 (m, 1H), 8.21 (br.s, 1H), 8.37 (br.s, 1H), 8.44-8.79 (m, 2H), 9.21 (br.s, 1H), 9.46 (br.s, 1H), 11.08 (s, 1H).

Table 30 summarizes the compounds prepared using General Procedure X-1.

TABLE 30
Final Compounds Prepared via General Procedure X-1

| Compound | TMB | CBM | | |
|---|---|---|---|---|
| No. | Portion | Portion | Structure | Characterization |
| P-1 | A1 | C1 | 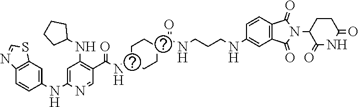 [SMILES: O=C1CCC(N2C(=O)c3ccc(NCCCNC(=O)C4CCC(NC(=O)c5cnc(Nc6ccc7ncsc7c6)cc5NC5CCCC5)CC4)cc3C2=O)C(=O)N1] | 14% yield as a yellow solid. LCMS method 2: retention time: 2.642 min, 97.0% purity at 215 nm, [M + H]⁺ = 792.3, [M + 2H]²⁺ = 396.6. ¹H NMR (400 MHz, DMSO-d6) δ ppm 1.25 − 1.48 (m, 6 H), 1.58 − 1.78 (m, 8 H), 1.83 − 1.90 (m, 2 H), 1.95 − 2.09 (m, 4 H), 2.53 − 2.61 |
| | | | | (m, 2 H), 2.81 − 2.93 (m, 1 H), 3.11 − 3.21 |
| | | | | (m, 4 H), 3.61 − 3.75 (m, 2 H), 5.03 (dd, |
| | | | | J = 13.0, 5.4 Hz, 1 H), 6.02 (s, 1 H), 6.85 |
| | | | | (d, J = 8.6 Hz, 1 H), 6.94 (s, 1 H), 7.10 (t, |
| | | | | J = 5.0 Hz, 1 H), 7.53 − 7.59 (m, 2 H), 7.81 |
| | | | | (t, J = 5.0 Hz, 1 H), 7.93 (d, J = 8.8 Hz, 1 |
| | | | | H), 7.99 (d, J = 7.8 Hz, 1 H), 8.34 (d, J = |
| | | | | 6.1 Hz, 1 H), 8.38 (s, 1 H), 8.69 (s, 1 H), |
| | | | | 9.14 (s, 1 H), 9.24 (s, 1 H), 11.06 (s, 1 H) |
| P-2 | A1 | C2 | 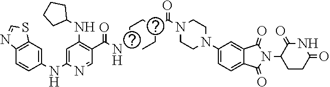 [SMILES: O=C1CCC(N2C(=O)c3ccc(N4CCN(C(=O)C5CCC(NC(=O)c6cnc(Nc7ccc8ncsc8c7)cc6NC6CCCC6)CC5)CC4)cc3C2=O)C(=O)N1] | 49% yield as a yellow solid. LCMS method 2: retention time: 2.701 min, 99.4% purity at 215 nm, [M + H]⁺ = 804.3, [M + 2H]²⁺ = 402.7. ¹H NMR (400 MHz, DMSO-d6) δ ppm 1.35 − 1.53 (m, 6 H), 1.58 − 1.71 (m, 4 H), |
| | | | | 1.73 − 1.80 (m, 2 H), 1.84 − 1.92 (m, 2 H), |
| | | | | 1.95 − 2.06 (m, 4 H), 2.53 − 2.64 (m, 2 H), |
| | | | | 2.83 − 2.94 (m, 1 H), 3.45 − 3.50 (m, 2 H), |
| | | | | 3.50 − 3.56 (m, 2 H), 3.59 − 3.65 (m, 2 H), |
| | | | | 3.65 − 3.75 (m, 4 H), 5.08 (dd, J = 12.7, |
| | | | | 5.1 Hz, 1 H), 6.03 (s, 1 H), 7.23 − 7.29 |
| | | | | (m, 1 H), 7.35 (d, J = 1.7 Hz, 1 H), 7.56 |
| | | | | (dd, J = 8.8, 2.0 Hz, 1 H), 7.71 (d, J = 8.6 |
| | | | | Hz, 1 H), 7.93 (d, J = 8.8 Hz, 1 H), 8.04 (d, |
| | | | | J = 7.6 Hz, 1 H), 8.33 (d, J = 6.4 Hz, 1 H), |
| | | | | 8.43 (s, 1 H), 8.70 (d, J = 2.0 Hz, 1 H), |
| | | | | 9.14 (s, 1 H), 9.27 (s, 1 H), 11.08 (s, 1 H). |
| P-4 | A2 | C2 | 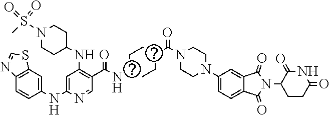 [SMILES: CS(=O)(=O)N1CCC(Nc2cc(Nc3ccc4ncsc4c3)ncc2C(=O)NC2CCC(C(=O)N3CCN(c4ccc5c(c4)C(=O)N(C4CCC(=O)NC4=O)C5=O)CC3)CC2)CC1] | 31% yield as a yellow solid. LCMS method 2: retention time: 2.456 min, 99.9% purity at 215 nm, [M + H]⁺ = 897.1, [M + 2H]²⁺ = 449.2. ¹H (400 MHz, DMSO-d6) δ ppm 1.34 − 1.61 (m, 6 H), 1.71 − 1.81 (m, 2 H), 1.84 − 1.96 (m, 2 H), 1.97 − 2.10 (m, 3 H), 2.52 − 2.69 (m, 3 H), 2.81 − 3.02 (m, 6 H), 3.40 − 3.57 |
| | | | | (m, 7 H), 3.57 − 3.65 (m, 2 H), 3.65 − 3.75 |
| | | | | (m, 3 H), 5.03 − 5.14 (m, 1 H), 6.09 (s, 1 H), |
| | | | | 7.26 (d, J = 8.3 Hz, 1 H), 7.35 (s, 1 H), 7.53 |
| | | | | (d, J = 8.6 Hz, 1 H), 7.71 (d, J = 8.6 Hz, 1 |
| | | | | H), 7.93 − 8.05 (m, 1 H), 8.21 (br.s, 1 H), |
| | | | | 8.37 (br.s, 1 H), 8.44 − 8.79 (m, 2 H), 9.21 |
| | | | | (br.s, 1 H), 9.46 (br.s, 1 H), 11.08 (s, 1 H). |
| P-6 | A4 | C2 | 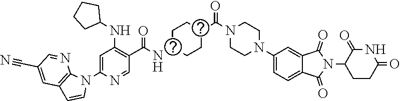 [SMILES: N#Cc1cnc2c(ccn2-c2cc(NC3CCCC3)c(C(=O)NC3CCC(C(=O)N4CCN(c5ccc6c(c5)C(=O)N(C5CCC(=O)NC5=O)C6=O)CC4)CC3)cn2)c1] | 38% yield as a yellow solid. LCMS method 2: retention time: 2.525 min, 99.6% purity at 215 nm, [M + H]⁺ = 797.2, [M + 2H]²⁺ = 399.2. ¹H (400 MHz, DMSO-d6) δ ppm 1.37 − 1.61 (m, 6 H), 1.62 − 1.83 (m, 6 H), 1.87 − 1.96 (m, 2 H), 1.98 − 2.06 (m, 1 H), 2.09 − |
| | | | | 2.19 (m, 2 H), 2.51 − 2.65 (m, 3 H), 2.80 − |
| | | | | 2.96 (m, 1 H), 3.43 − 3.57 (m, 4 H), 3.58 − |
| | | | | 3.66 (m, 2 H), 3.66 − 3.79 (m, 3 H), 3.87 − |
| | | | | 3.97 (m, 1 H), 5.08 (dd, J = 12.9, 5.3 Hz, |
| | | | | 1 H), 6.89 (d, J = 3.9 Hz, 1 H), 7.26 (d, J = |
| | | | | 8.6 Hz, 1 H), 7.35 (s, 1 H), 7.71 (d, J = 8.6 |
| | | | | Hz, 1 H), 8.15 (s, 1 H), 8.36 (d, J = 7.3 Hz, |
| | | | | 1 H), 8.54 (d, J = 3.8 Hz, 1 H), 8.58 (s, 1 |
| | | | | H), 8.64 − 8.70 (m, 2 H), 8.83 (d, |
| | | | | J = 1.8 Hz, 1 H), 11.08 (s, 1 H). |
| P-7 | A5 | C2 | 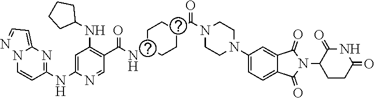 [SMILES: O=C1CCC(N2C(=O)c3ccc(N4CCN(C(=O)C5CCC(NC(=O)c6cnc(Nc7ccn8nccc8n7)cc6NC6CCCC6)CC5)CC4)cc3C2=O)C(=O)N1] | 13% yield as a yellow solid. LCMS method 2: retention time: 2.576 min, 98.8% purity at 215 nm, [M + H]⁺ = 788.3, [M + 2H]²⁺ = 394.7. ¹H (400 MHz, DMSO-d6) δ ppm 1.35 − 1.57 (m, 7 H), 1.63 − 1.81 (m, 6 H), 1.84 − 1.93 (m, 2 H), 1.98 − 2.18 (m, |
| | | | | 3 H), 2.53 − 2.64 (m, 2 H), 2.82 − |
| | | | | 2.96 (m, 1 H), 3.44 − 3.56 (m, 4 H), |
| | | | | 3.59 − 3.74 (m, 5 H), 3.81 (q, J = 6.1 |
| | | | | Hz, 1 H), 5.08 (dd, J = 12.8, 5.3 Hz, |
| | | | | 1 H), 6.18 (d, J = 1.5 Hz, 1 H), 6.93 |
| | | | | (d, J = 7.6 Hz, 1 H), 7.26 (d, J = 8.6 |
| | | | | Hz, 1 H), 7.35 ( s, 1 H), 7.71 (d, J = |
| | | | | 8.6 Hz, 1 H), 7.88 (s, 1 H), 7.96 (d, |
| | | | | J = 2.0 Hz, 1 H), 8.11 − 8.18 (m, 1 H), |
| | | | | 8.41 (s, 1 H), 8.55 (d, J = 5.6 Hz, 1 H), |
| | | | | 8.76 (d, J = 7.6 Hz, 1 H), 10.14 (s, |
| | | | | 1 H), 11.08 (s, 1 H). |
| P-8 | A1 | C4 | 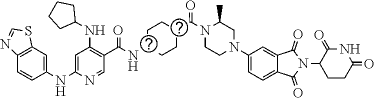 [SMILES: C[C@H]1CN(c2ccc3c(c2)C(=O)N(C2CCC(=O)NC2=O)C3=O)CCN1C(=O)C1CCC(NC(=O)c2cnc(Nc3ccc4ncsc4c3)cc2NC2CCCC2)CC1] | 35% yield as a yellow solid. LCMS method 2: retention time: 2.715 min, 97.0% purity at 215 nm, [M + H]⁺ = 818.3, [M + 2H]²⁺ = 409.8 ¹H ( 400 MHz, DMSO-d6) δ ppm 1.04 − 1.14 ( m, 2 H), 1.21 − 1.29 ( m, 1 H), 1.33 − 1.54 ( m, 6 H), 1.56 − 1.80 ( m, 7 |
| | | | | H), 1.83 − 1.94 ( m, 2 H), 1.95 − 2.06 |
| | | | | (m, 3 H), 2.53 − 2.64 (m, 2 H), 2.83 − |
| | | | | 3.01 (m, 1 H), 3.06 − 3.22 (m, 1 H), |
| | | | | 3.50 − 3.61 (m, 1 H), 3.63 − 3.77 (m, 2 |
| | | | | H), 3.80 − 3.99 (m, 3 H), 4.17 − 4.39 (m, |
| | | | | 1 H), 4.51 − 4.63 (m, 1 H), 5.07 (dd, J = |
| | | | | 12.8, 5.3 Hz, 1 H), 6.03 (s, 1 H), 7.17 − |
| | | | | 7.25 (m, 1 H), 7.28 − 7.36 (m, 1 H), |
| | | | | 7.56 (dd, J = 9.0, 1.4 Hz, 1 H), 7.69 (d, |
| | | | | J = 8.4 Hz, 1 H), 7.94 (d, J = 8.8 Hz, 1 |
| | | | | H), 8.04 (d, J = 7.0 Hz, 1 H), 8.33 (d, |
| | | | | J = 5.9 Hz, 1 H), 8.40 (s, 1 H), 8.70 (s, |
| | | | | 1 H), 9.14 (s, 1 H), 9.25 (s, 1 H), 11.08 |
| | | | | (s, 1 H). |
| | | | | Rotamers observed by ¹H-NMR. |
| P-10 | A7 | C5 | 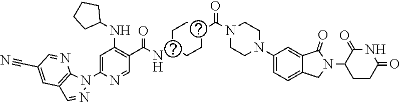 [SMILES: N#Cc1cnc2c(cnn2-c2cc(NC3CCCC3)c(C(=O)NC3CCC(C(=O)N4CCN(c5ccc6c(c5)C(=O)N(C5CCC(=O)NC5=O)C6)CC4)CC3)cn2)c1] | 92% yield as an off-white solid. LCMS method 2: retention time: 3.180 min, 99.5% purity at 215 nm, [M + H]⁺ = 784.3, [M + 2H]²⁺ = 392.8 ¹H (400 MHz, DMSO-d6) δ ppm 1.35 − 1.57 (m, 6 H), 1.58 − 1.74 (m, 4 H), 1.74 − |
| | | | | 1.84 (m, 2 H), 1.94 (s, 2 H), 1.96 − 2.12 |
| | | | | (m, 3 H), 2.30 − 2.44 (m, 1 H), 2.62 (m, |
| | | | | 2 H), 2.84 − 2.99 (m, 1 H), 3.12 − 3.31 |
| | | | | (m, 4 H), 3.56 − 3.72 (m, 4 H), 3.72 − 3.81 |
| | | | | (m, 1 H), 3.85 − 3.97 (m, 1 H), 4.18 − 4.27 |
| | | | | (m, 1 H), 4.30 − 4.41 (m, 1 H), 5.10 (dd, |
| | | | | J = 13.2, 4.9 Hz, 1 H), 7.21 (s, 1 H), 7.25 − |
| | | | | 7.38 (m, 2 H), 7.46 (d, J = 8.3 Hz, 1 H), |
| | | | | 8.44 (d, J = 7.1 Hz, 1 H), 8.56 − 8.70 (m, |
| | | | | 3 H), 9.02 (s, 1 H), 9.05 (s, 1 H). |
| | | | | One proton was not apparent by ¹H. |
| P-11 | A1 | C6 | 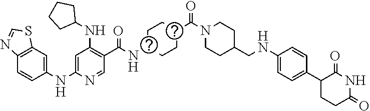 [SMILES: O=C1CCC(c2ccc(NCC3CCN(C(=O)C4CCC(NC(=O)c5cnc(Nc6ccc7ncsc7c6)cc5NC5CCCC5)CC4)CC3)cc2)C(=O)N1] | 8.5% yield as an off-white solid. LCMS method 2: retention time: 2.612 min, 98.9% purity at 215 nm, [M + H]⁺ = 763.3, [M + 2H]²⁺ = 382.3 ¹H (400 MHz, DMSO-d6) δ ppm 0.92 − 1.13 (m, 2 H), 1.36 − 1.51 (m, 6 H), 1.59 − 1.89 (m, 12 H), 1.96 − 2.04 (m, 3 H), 2.55 − 2.65 (m, 2 H), 2.88 − 2.93 |
| | | | | (m, 2 H), 2.94 − 3.04 (m, 1 H), 3.58 − |
| | | | | 3.77 (m, 4 H), 3.92 − 4.01 (m, 1 H), |
| | | | | 4.38 − 4.46 (m, 1 H), 5.61 − 5.67 (m, |
| | | | | 1 H), 6.02 (s, 1 H), 6.53 (d, J = 8.1 |
| | | | | Hz, 2 H), 6.89 (d, J = 8.1 Hz, 2 H), |
| | | | | 7.56 (d, J = 8.5 Hz, 1 H), 7.93 (d, J = |
| | | | | 8.0 Hz, 1 H), 8.02 (d, J = 8.0 Hz, 1 H), |
| | | | | 8.32 (d, J = 5.0 Hz, 1 H), 8.39 (s, 1 H), |
| | | | | 8.70 (s, 1 H), 9.14 (s, 1 H), 9.24 |
| | | | | (s, 1 H), 10.73 (s, 1 H). |
| | | | | Two protons partially obscured by |
| | | | | solvent peak and one exchangeable |
| | | | | proton was not apparent by ¹H. |
| P-12 | A6 | C2 | 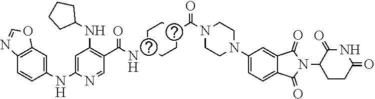 [SMILES: O=C1CCC(N2C(=O)c3ccc(N4CCN(C(=O)C5CCC(NC(=O)c6cnc(Nc7ccc8ncoc8c7)cc6NC6CCCC6)CC5)CC4)cc3C2=O)C(=O)N1] | 4.5% yield as an yellow solid. LCMS method 9: retention time: 3.852 min, 96.4% purity at 215 nm, [M − HCOOH + 2H]²⁺ = 394.8 ; [M − HCOOH + H]⁺ = 788.2 ¹H (400 MHz, DMSO-d6 ) δ ppm 0.94 − 1.33 (m, 5 H), 1.34 − 1.53 (m, 4 H), |
| | | | | 1.54 − 1.82 (m, 5 H), 1.83 − 1.92 (m, 2 |
| | | | | H), 1.95 − 2.09 (m, 2 H), 2.54 − 2.65 (m, |
| | | | | 2 H), 2.82 − 2.91 (m, 1 H), 3.40-3.75 (m, |
| | | | | 8 H), 3.89 − 4.06 (m, 1 H), 4.07 − 4.19 |
| | | | | (m, 1 H), 5.08 (dd, J = 12.8, 5.3 Hz, 1 |
| | | | | H), 6.01 (s, 1 H), 6.77 − 6.87 (m, 1 H), |
| | | | | 7.03 − 7.12 (m, 1 H), 7.22 − 7.41 (m, 2 |
| | | | | H), 7.58 − 7.69 (m, 1 H), 7.71 (d, J = |
| | | | | 8.6 Hz, 1 H), 8.33 (s, 1 H), 8.38 (s, 1 H), |
| | | | | 8.47 (s, 1 H), 8.56 (s, 1 H), 9.25 (br. s, |
| | | | | 1 H), 11.08 (s, 1 H). |
| P-13 | A7 | C7 | 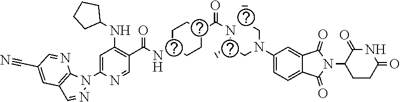 [SMILES: C[C@@H]1CN(c2ccc3c(c2)C(=O)N(C2CCC(=O)NC2=O)C3=O)C[C@H](C)N1C(=O)C1CCC(NC(=O)c2cnc(-n3ncc4cc(C#N)cnc43)cc2NC2CCCC2)CC1] | 58% yield as an yellow solid. LCMS method 2: retention time: 3.599 min, 99.1% purity at 215 nm, [M + H]+ = 826.3. ¹H (400 MHz, DMSO-d6) δ ppm 1.11 − 1.22 (m, 3 H), 1.27 − 1.39 (m, 3 H), 1.39 − 1.58 (m, 5 H), |
| | | | | 1.58 − 1.75 (m, 5 H), 1.76 − 1.88 |
| | | | | (m, 2 H), 1.89 − 1.98 (m, 2 H), |
| | | | | 1.99 − 2.13 (m, 3 H), 2.54 − 2.64 |
| | | | | (m, 2 H), 2.84 − 2.95 (m, 1 H), |
| | | | | 3.13 − 3.21 (m, 1 H), 3.22 − 3.29 |
| | | | | (m, 2 H), 3.71 − 3.82 (m, 1 H), |
| | | | | 3.87 − 3.95 (m, 1 H), 3.97 − 4.07 |
| | | | | (m, 2 H), 4.24 − 4.35 (m, 1 H), |
| | | | | 4.50 − 4.62 (m, 1 H), 5.08 (br. dd, |
| | | | | J = 13.0, 5.1 Hz, 1 H), 7.27 − 7.32 |
| | | | | (m, 1 H), 7.34 (s, 1 H), 7.37 − 7.40 |
| | | | | (m, 1 H), 7.67 − 7.73 (m, 1 H), |
| | | | | 8.38 − 8.44 (m, 1 H), 8.57 − 8.63 |
| | | | | (m, 2 H), 8.65 (s, 1 H), 9.05 (dd, |
| | | | | J = 14.9, 2.0 Hz, 2 H), 11.09 (s, 1 H). |
| P-14 | A7 | C8 | 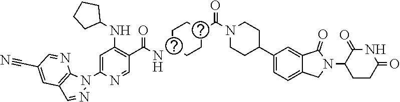 [SMILES: N#Cc1cnc2c(cnn2-c2cc(NC3CCCC3)c(C(=O)NC3CCC(C(=O)N4CCC(c5ccc6c(c5)C(=O)N(C5CCC(=O)NC5=O)C6)CC4)CC3)cn2)c1] | 38% yield as a white solid. LCMS method 2: retention time: 3.361 min, 98.5% purity at 215 nm, [M + H]⁺ = 783.3, [M + 2H]²⁺ = 392.3 ¹H (400 MHz, DMSO-d6) δ ppm 1.39 − 1.55 (m, 7 H), 1.58 − 1.73 (m, 5 H), 1.75 − 1.96 (m, 6 H), 1.97 − 2.09 (m, 3 |
| | | | | H), 2.32 − 2.42 (m, 1 H), 2.55 − 2.66 |
| | | | | (m, 3 H), 2.85 − 2.98 (m, 2 H), 3.09 − |
| | | | | 3.20 (m, 1 H), 3.70 − 3.80 (m, 1 H), |
| | | | | 3.86 − 3.94 (m, 1 H), 4.06 − 4.15 (m, 1 |
| | | | | H), 4.24 − 4.32 (m, 1 H), 4.38 − 4.45 (m, |
| | | | | 1 H), 4.59 (br. d, J = 11.7 Hz, 1 H), |
| | | | | 5.11 (dd, J = 13.1, 5.0 Hz, 1H), 7.33 (s, |
| | | | | 1 H), 7.54 (s, 2 H), 7.60 (s, 1 H), 8.42 |
| | | | | (d, J = 7.6 Hz, 1 H), 8.58 − 8.62 (m, 2 |
| | | | | H), 8.64 (s, 1 H), 9.01 (d, J = 2.0 Hz, 1 |
| | | | | H), 9.05 (d, J = 2.0 Hz, 1 H), 10.98 (s, 1 |
| | | | | H). |
| P-20 | A7 | C10 | 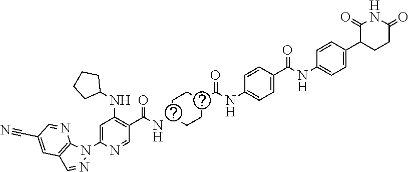 [SMILES: N#Cc1cnc2c(cnn2-c2cc(NC3CCCC3)c(C(=O)NC3CCC(C(=O)Nc4ccc(C(=O)Nc5ccc(C6CCC(=O)NC6=O)cc5)cc4)CC3)cn2)c1] | 43% yield as a white solid. LCMS method 2: retention time: 3.361 min, 99.9% purity at 215 nm, [M + H]⁺ = 779.2, [M + 2H]²⁺ = 390.2 ¹H (400 MHz, DMSO-d6) δ ppm 1.36 − 1.57 (m, 6 H), 1.58 − 1.77 (m, 5 H), 1.92 − 2.01 (m, 4 H), 2.02 − 2.12 (m, 3 H), 2.13 − 2.26 (m, 1 H), 2.35 − 2.43 (m, 1 H), 2.61 − 2.73 (m, 1 H), 3.75 − 3.86 (m, 2 H), 3.87 − 3.95 (m, 1 H), 7.19 (d, J = 8.6 Hz, 2 H), 7.35 (s, 1 H), 7.70 − 7.77 (m, 4 H), 7.91 − |
| | | | | 7.97 (m, 2 H), 8.40 − 8.45 (m, 1 H), 8.61 − |
| | | | | 8.67 (m, 3 H), 9.01 − 9.08 (m, 2 H), 10.09 − |
| | | | | 10.18 (m, 2 H), 10.81 − 10.84 (m, 1 H). |
| P-21 | A7 | C10 | 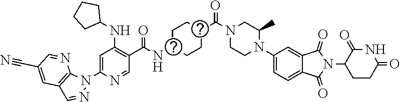 [SMILES: C[C@@H]1CN(C(=O)C2CCC(NC(=O)c3cnc(-n4ncc5cc(C#N)cnc54)cc3NC3CCCC3)CC2)CCN1c1ccc2c(c1)C(=O)N(C1CCC(=O)NC1=O)C2=O] | 46% yield as a yellow solid. LCMS method 2: retention time: 5.293 min, 96.6% purity at 215 nm, [M + H]⁺ = 812.3, [M + 2H]²⁺ = 406.8 ¹H (400 MHz, DMSO-d6) δ ppm 0.99 (d, J = 6.2 Hz, 1.5 H), 1.11 (d, J = 6.2 Hz, 1.5 H), 1.35 − 1.82 (m, 11 |
| | | | | H), 1.83 − 1.90 (m, 1 H), 1.90 -1.98 (m, 2 |
| | | | | H), 1.98 − 2.12 (m, 3 H), 2.54 − 2.64 (m, 2 |
| | | | | H), 2.69 − 2.75 (m, 0.5 H), 2.81 − 2.96 (m, |
| | | | | 1 H), 2.96 − 3.09 (m, 1 H), 3.12 − 3.31 (m, |
| | | | | 1.5 H), 3.35 − 3.45 (m, 0.5 H), 3.54 − 3.62 |
| | | | | (m, 0.5 H), 3.69 − 3.83 (m, 2 H), 3.86 − 3.96 |
| | | | | (m, 1.5 H), 4.02 − 4.12 (m, 0.5 H), 4.20 − |
| | | | | 4.31 (m, 1 H), 4.32 − 4.44 (m, 1 H), 5.08 |
| | | | | (dd, J = 13.0, 5.4 Hz, 1 H), 7.23 (d, J = 8.8 |
| | | | | Hz, 1 H), 7.31 (s, 1 H), 7.34 (s, 1 H), 7.72 |
| | | | | (d, J = 8.6 Hz, 1 H), 8.40 − 8.47 (m, 1 H), |
| | | | | 8.57 − 8.64 (m, 2 H), 8.65 (s, 1 H), 9.03 (d, |
| | | | | J = 2.0 Hz, 1 H), 9.06 (d, J = 2.0 Hz, 1 H), |
| | | | | 11.09 (s, 1 H). |
| | | | | Rotamers were observed by 1H. |
| P-22 | A10 | C5 | 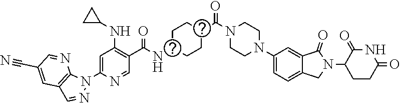 [SMILES: N#Cc1cnc2c(cnn2-c2cc(NC3CC3)c(C(=O)NC3CCC(C(=O)N4CCN(c5ccc6c(c5)C(=O)N(C5CCC(=O)NC5=O)C6)CC4)CC3)cn2)c1] | 18% yield as an off-white solid. LCMS method 2: retention time: 2.979 min, 99.9% purity at 215 nm, [M + H]⁺ = 756.3, [M + 2H]²⁺ = 378.7 ¹H (400 MHz, DMSO-d6 ) δ ppm 0.54 − 0.61 (m, 2 H), 0.81 − 0.90 |
| | | | | (m, 2 H), 1.39 − 1.55 (m, 4 H), |
| | | | | 1.73 − 1.83 (m, 2 H), 1.88 − 2.04 |
| | | | | (m, 3 H), 2.35 − 2.45 (m, 1 H), |
| | | | | 2.55 − 2.64 (m, 2 H), 2.85 − 2.97 |
| | | | | (m, 1 H), 3.11 − 3.28 (m, 5 H), |
| | | | | 3.59 − 3.80 (m, 5 H), 4.18 − 4.28 |
| | | | | (m, 1 H), 4.31 − 4.40 (m, 1 H), |
| | | | | 5.10 (dd, J = 13.0, 5.4 Hz, 1 H), |
| | | | | 7.21 (s, 1 H), 7.27 − 7.33 (m, 1 |
| | | | | H), 7.46 (d, J = 8.6 Hz, 1 H), 7.67 |
| | | | | (s, 1 H), 8.45 (d, J = 7.6 Hz, 1 H), |
| | | | | 8.52 (s, 1 H), 8.61 (s, 1 H), 8.66 |
| | | | | (s, 1 H), 9.01 − 9.05 (m, 1 H), 9.05 − |
| | | | | 9.09 (m, 1 H), 10.98 (br. s, 1 H). |
| P-23 | A7 | C12 | 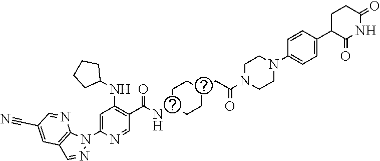 [SMILES: N#Cc1cnc2c(cnn2-c2cc(NC3CCCC3)c(C(=O)NC3CCC(CC(=O)N4CCN(c5ccc(C6CCC(=O)NC6=O)cc5)CC4)CC3)cn2)c1] Use trans-2-(4-Aminocyclohexyl)acetic acid in first step | 47% yield as a white solid. LCMS method 2: retention time: 3.395 min, 96.0% purity at 215 nm, [M + H]⁺ = 743.3, [M + 2H]²⁺ = 372.3 ¹H (400 MHz, DMSO-d6) δ ppm 1.00 − 1.17 (m, 2 H), 1.28 − 1.43 (m, 2 H), 1.51 (dq, J = 11.7, 5.8 Hz, 2 H), 1.58 − 1.75 (m, 5 H), 1.75 − 1.92 (m, 4 H), 1.96 − 2.20 (m, 4 H), 2.28 (d, J = 6.6 Hz, 2 H), 2.44 (t, J = 4.2 Hz, 1 H), 2.56 − 2.71 (m, 1 H), 3.03 − 3.18 (m, 4 H), 3.55 − 3.66 (m, 4 H), 3.74 (dd, J = 11.0, 4.9 Hz, 2 H), 3.89 |
| | | | | (dq, J = 12.0, 6.0 Hz, 1 H), 6.92 (d, |
| | | | | J = 8.6 Hz, 2 H), 7.08 (d, J = 8.6 Hz, |
| | | | | 2 H), 7.33 (s, 1 H), 8.37 (d, J = |
| | | | | 7.6 Hz, 1 H), 8.60 (s, 1 H), 8.64 (s, |
| | | | | 2 H), 9.01 (d, J = 2.0 Hz, 1 H), 9.05 |
| | | | | (d, J = 2.0 Hz, 1 H), 10.78 (s, 1 H). |
| P-25 | A11 | C5 | 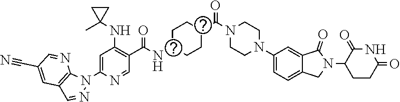 [SMILES: CC1(Nc2cc(-n3ncc4cc(C#N)cnc43)ncc2C(=O)NC2CCC(C(=O)N3CCN(c4ccc5c(c4)C(=O)N(C4CCC(=O)NC4=O)C5)CC3)CC2)CC1] | 41% yield as a white solid. LCMS method 2: retention time: 3.009 min, 98.2% purity at 215 nm, [M + H]⁺ = 770.3, [M + 2H]²⁺ = 385.7 ¹H (400 MHz, DMSO-d6 ) δ ppm 0.75 − 0.84 (m, 4 H), 1.39 − 1.54 (m, 7 H), 1.73 − 1.82 (m, 2 H), 1.89 − 2.03 (m, 3 |
| | | | | H), 2.31 − 2.45 (m, 1 H), 2.53 − 2.68 |
| | | | | (m, 2 H), 2.86 − 2.96 (m, 1 H), 3.13 − |
| | | | | 3.27 (m, 4 H), 3.60 − 3.79 (m, 5 H), |
| | | | | 4.18 − 4.27 (m, 1 H), 4.30 − 4.40 (m, 1 |
| | | | | H), 5.10 (dd, J = 13.2, 5.1 Hz, 1 H), |
| | | | | 7.19 − 7.23 (m, 1 H), 7.30 (dd, J = 8.4, |
| | | | | 1.6 Hz, 1 H), 7.46 (d, J = 8.6 Hz, 1 H), |
| | | | | 7.67 (s, 1 H), 8.43 (d, J = 7.6 Hz, 1 H), |
| | | | | 8.61 (s, 1 H), 8.66 (s, 1 H), 8.69 (s, 1 |
| | | | | H), 9.03 (d, J = 1.7 Hz, 1 H), 9.06 (d, J = |
| | | | | 1.7 Hz, 1 H), 10.97 (s, 1 H). |
| P-26 | A12 | C5 | 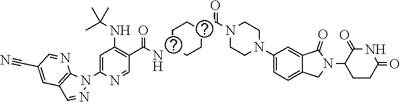 [SMILES: CC(C)(C)Nc1cc(-n2ncc3cc(C#N)cnc32)ncc1C(=O)NC1CCC(C(=O)N2CCN(c3ccc4c(c3)C(=O)N(C3CCC(=O)NC3=O)C4)CC2)CC1] | 30% yield as a white solid. LCMS method 2: retention time: 2.991 min, 99.4% purity at 215 nm, [M + H]⁺ = 772.3, [M + 2H]²⁺ = 386.7 ¹H (400 MHz, DMSO-d6 ) δ ppm 1.40 − 1.55 (m, 13 H), 1.73 − 1.83 (m, 2 H), 1.90 − 2.04 (m, 3 H), 2.31 − 2.45 (m, 1 H), 2.56 − 2.68 |
| | | | | (m, 2 H), 2.86 − 2.96 (m, 1 H), 3.13 − 3.27 |
| | | | | (m, 4 H), 3.60 − 3.79 (m, 5 H), 4.18 − 4.27 |
| | | | | (m, 1 H), 4.30 − 4.40 (m, 1 H), 5.10 (dd, J = |
| | | | | 13.3, 5.0 Hz, 1 H), 7.19 − 7.23 (m, 1 H), |
| | | | | 7.30 (dd, J = 8.6, 1.7 Hz, 1 H), 7.46 (d, J = |
| | | | | 8.3 Hz, 1 H), 7.62 (s, 1 H), 8.43 (d, J = 7.3 |
| | | | | Hz, 1 H), 8.60 (s, 1 H), 8.65 (s, 1 H), 8.83 (s, |
| | | | | 1 H), 9.02 (d, J = 2.0 Hz, 1 H), 9.05 (d, J = |
| | | | | 2.0 Hz, 1 H), 10.97 (s, 1 H). |
| P-28 | A7 | C14 | 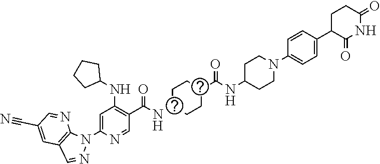 [SMILES: N#Cc1cnc2c(cnn2-c2cc(NC3CCCC3)c(C(=O)NC3CCC(C(=O)NC4CCN(c5ccc(C6CCC(=O)NC6=O)cc5)CC4)CC3)cn2)c1] | 44% yield as a tan solid. LCMS method 2: retention time: 2.825 min, 98.9% purity at 215 nm, [M + H]⁺ = 743.3, [M + 2H]²⁺ = 372.2 ¹H (400 MHz, DMSO-d6 ) δ ppm 1.28 − 1.57 (m, 8 H), 1.58 − 1.84 (m, 8 H), 1.87 − 1.95 (m, 2 H), 1.97 − 2.19 (m, 5 H), 2.41 − 2.47 (m, 1 H), 2.61 (d, J = 5.6 Hz, 1 H), 2.72 − 2.86 (m, 2 H), 3.59 − 3.80 (m, 5 H), 3.86 − 3.95 (m, 1 H), 6.92 (br. s, 2 H), 7.05 (d, J = 7.6 |
| | | | | Hz, 2 H), 7.36 (s, 1 H), 7.71 (d, J = |
| | | | | 6.6 Hz, 1 H), 8.39 (d, J = 7.6 Hz, |
| | | | | 1 H), 8.58 − 8.70 (m, 3 H), 9.00 − |
| | | | | 9.08 (m, 2 H), 10.77 (s, 1 H). |
| P-129 | A-10 | C-13 | 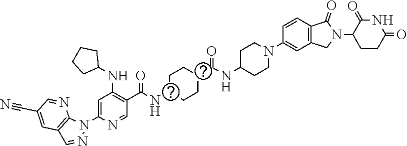 [SMILES: N#Cc1cnc2c(cnn2-c2cc(NC3CCCC3)c(C(=O)NC3CCC(C(=O)NC4CCN(c5ccc6c(c5)CN(C5CCC(=O)NC5=O)C6=O)CC4)CC3)cn2)c1] | 20% yield as a tan solid. LCMS method 2: retention time: 2.874 min, 94.4% purity at 215 nm, [M + H]⁺ = 770.4, [M + 2H]²⁺ = 385.8 ¹H (400 MHz, DMSO-d6 ) δ ppm 0.53 − 0.59 (m, 2 H), 0.83 − 0.87 (m, 2 H), 1.12 − 1.18 (m, 1 H), 1.31 − 1.51 (m, 6 H), 1.74 − 1.84 (m, 4 H), 1.86 − 2.00 (m, 3 H), 2.02 − 2.11 (m, 1 H), 2.32 − 2.42 (m, 1 H), 2.54 − 2.61 (m, 2 H), 2.87 − 3.03 (m, 3 H), 3.70 − |
| | | | | 3.83 (m, 3 H), 4.15 − 4.36 (m, 2 H), 5.04 |
| | | | | (dd, J = 13.2, 5.1 Hz, 1 H), 7.01 − 7.09 (m, |
| | | | | 2 H), 7.50 (d, J = 8.3 Hz, 1 H), 7.67 (s, 1 |
| | | | | H), 7.70 (br d, J = 7.8 Hz, 1 H), 8.36 − |
| | | | | 8.47 (m, 1 H), 8.54 (s, 1H), 8.59 (s, 1 H), |
| | | | | 8.65 (s, 1 H), 9.02 (d, J = 2.2 Hz, 1 H), |
| | | | | 9.05 (d, J = 2.0 Hz, 1 H), 10.93 (br s, 1 H). |

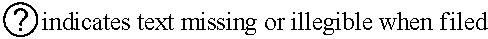

### General Procedure X-2

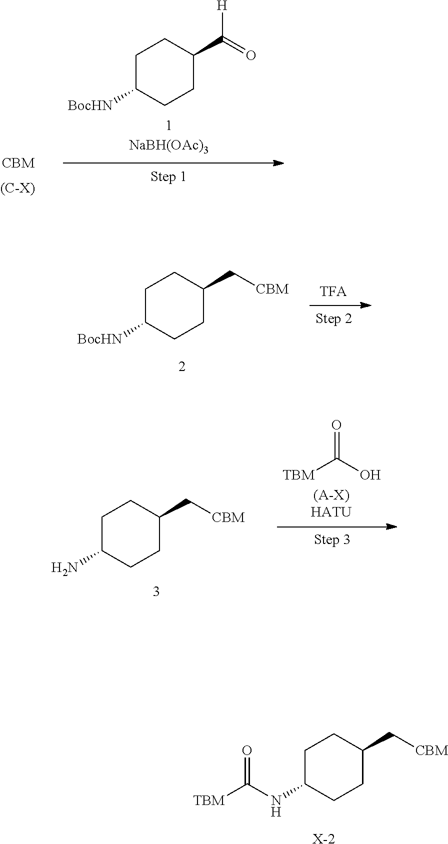

Step 1. Preparation of 2 via reductive amination between CBM amine (C-X) and tert-butyl (4-formylcyclohexyl)carbamate: To a round bottom flask was added tert-butyl N-(4-formylcyclohexyl)carbamate 1 (1.1 eq.) and AcOH (8 eq.) in DCM (0.11 M) and MeOH (0.11 M). The resulting solution was stirred for 20 minutes, after which CBM amine (C-X) TFA salt (1 eq.) and NaBH(OAc)₃ (2.5 eq.) were successively added to the reaction mixture. The resulting solution was stirred at room temperature. After LCMS showed complete conversion of the starting material into compound 2. The solvent was evaporated under reduce pressure and the crude mixture was purified. Fractions were combined and concentrated to give 2.

Step 2. Preparation of 3 via Boc deprotection with trifluoroacetic acid: To a solution of 2 (1 eq.) in DCM (0.11 M) was added TFA (31 eq.). The reaction mixture was stirred at room temperature. After 1 h, LCMS showed full conversion into compound 3. The reaction mixture was concentrated under reduced pressure and the residue purified to afford 3 as a trifluoroacetic acid salt.

Step 3. Preparation of final product via amide coupling of amine 3 and TBM carboxylic acid (GP-2): To a solution of TBM carboxylic acid (A-X) (1.11 eq.) and amine 3 TFA salt (1 eq.) in DMF (0.11 M) was added DIPEA (10.5 eq.). After 5 minutes of stirring at room temperature, HATU (1.2 eq.) was added. The solution was then stirred at room temperature. After LCMS showed complete conversion, the material was purified to give the final product (X-2).

### Example S41. 6-(1,3-benzothiazol-6-ylamino)-4-(cyclopentylamino)-N-[4-[[4-[2-(2,6-dioxo-3-piperidyl)-1,3-dioxo-isoindolin-5-yl]piperazin-1-yl]methyl]cyclohexyl]pyridine-3-carboxamide (P-3)

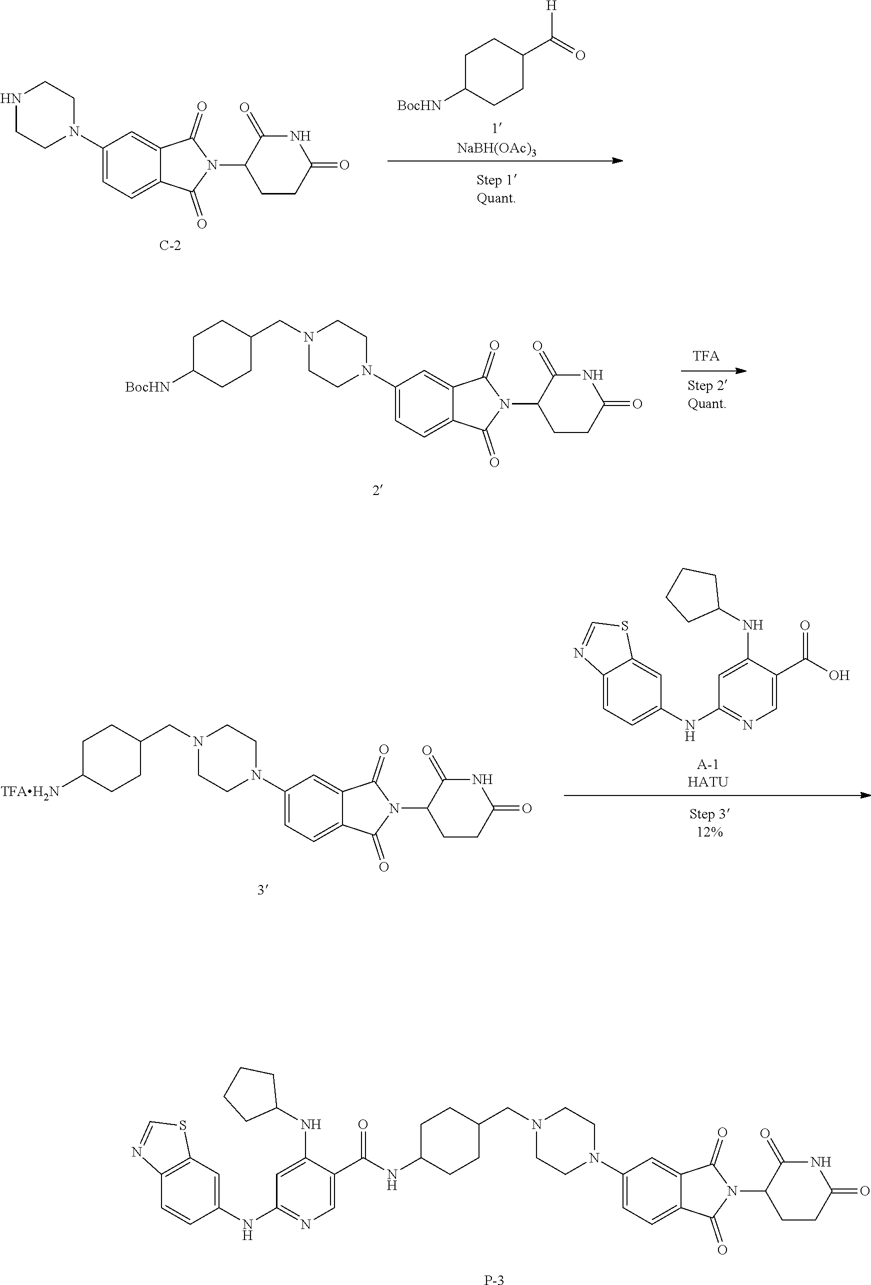

Step 1′. Preparation of tert-butyl N-[4-[[4-[2-(2,6-dioxo-3-piperidyl)-1,3-dioxo-isoindolin-5-yl]piperazin-1-yl]methyl]cyclohexyl]carbamate (2′): To a round bottom flask was added tert-butyl N-(4-formylcyclohexyl)carbamate 1′ (110 mg, 0.48 mmol, 1.1 eq.) and AcOH (0.2 mL, 3.49 mmol, 8 eq.) in DCM (2 mL, 0.11 M) and MeOH (2 mL, 0.11 M). The resulting solution was stirred for 20 minutes, after which 2-(2,6-dioxo-3-piperidyl)-5-piperazin-1-yl-isoindoline-1,3-dione;2,2,2-trifluoroacetic acid C-2 (150.33 mg, 0.44 mmol, 1 eq.) and NaBH(OAc)₃ (230 mg, 1.09 mmol, 2.5 eq.) were successively added to the reaction mixture. The resulting solution was stirred at room temperature. After 3 h, LCMS showed complete conversion of the starting material into compound 3. The solvent was evaporated under reduce pressure and the crude mixture was purified by reverse phase FC purification (30 g C18 column, liquid deposit (DMSO), 5% MeOH/0.1% HCOOH over 3 CV, then 5 to 100% MeOH/0.1% HCOOH over 20 CV, the product came out at 70% MeOH). Fractions were combined and concentrated to give 2′ (282 mg, quantitative yield) as a yellow solid that was directly used in the next step.

LCMS method 1: 99.9% purity at 215 nm, [M+H]⁺=554.4.

Step 2′. Preparation of 5-[4-[(4-aminocyclohexyl)methyl]piperazin-1-yl]-2-(2,6-dioxo-3-piperidyl)isoindoline-1,3-dione;2,2,2-trifluoroacetic acid (3′): To a solution of tert-butyl N-[4-[[4-[2-(2,6-dioxo-3-piperidyl)-1,3-dioxo-isoindolin-5-yl]piperazin-1-yl]methyl]cyclohexyl]carbamate 2′ (182.37 mg, 0.33 mmol, 1 eq.) in DCM (3 mL, 0.11 M) was added TFA (0.8 mL, 10.31 mmol, 31 eq.). The reaction mixture was stirred at room temperature. After 1 h, LCMS showed full conversion into compound 4. The reaction mixture was concentrated under reduced pressure and the residue was co-evaporated with Toluene (2×) and MeCN (2×) to give 3′ (227.8 mg, quantitative yield) as a yellow solid as a trifluoroacetic acid salt.

LCMS method 1: 99.9% purity at 215 nm, [M-CF₃COOH+H]⁺=454.4.

¹H NMR (400 MHz, DMSO-d6) δ ppm 1.06 (q, J=12.2 Hz, 2H), 1.29-1.39 (m, 3H), 1.72-1.80 (m, 1H), 1.81-1.89 (m, 3H), 1.94 (br d, J=11.5 Hz, 2H), 1.99-2.06 (m, 1H), 2.52-2.63 (m, 2H), 2.83-2.98 (m, 2H), 2.99-3.05 (m, 2H), 3.25-3.41 (m, 2H), 3.53-3.68 (m, 2H), 4.08-4.30 (m, 2H), 5.10 (dd, J=13.0, 5.4 Hz, 1H), 7.36 (dd, J=8.6, 2.0 Hz, 1H), 7.47-7.51 (m, 1H), 7.77 (d, J=8.6 Hz, 1H), 7.85-7.93 (m, 3H), 11.10 (s, 1H).

Step 3′. Preparation of 6-(1,3-benzothiazol-6-ylamino)-4-(cyclopentylamino)-N-[4-[[4-[2-(2,6-dioxo-3-piperidyl)-1,3-dioxo-isoindolin-5-yl]piperazin-1-yl]methyl]cyclohexyl]pyridine-3-carboxamide (P-3): To a solution of 6-(1,3-benzothiazol-6-ylamino)-4-(cyclopentylamino)pyridine-3-carboxylic acid A-1 (130 mg, 0.37 mmol, 1.11 eq.) and 5-[4-[(4-aminocyclohexyl)methyl]piperazin-1-yl]-2-(2,6-dioxo-3-piperidyl)isoindoline-1,3-dione;2,2,2-trifluoroacetic acid 3′ (186 mg, 0.33 mmol, 1 eq.) in DMF (3 mL, 0.11 M) was added DIPEA (0.6 mL, 3.44 mmol, 10.5 eq.). After 5 minutes of stirring at room temperature, HATU (150 mg, 0.39 mmol, 1.2 eq.) was added. The solution was then stirred at room temperature. After 2 h, LCMS showed complete conversion. The reaction was stopped and it was directly loaded for reverse phase FC purification (50 g C18 column, liquid deposit (reaction mixture), 5% MeOH/0.1% HCOOH over 4 CV, then 5 to 95% MeOH/0.1% HCOOH over 20 CV, the product came out at 60% MeOH). Fractions were combined and concentrated to give P-3 (31 mg, 12% yield) as a yellow solid.

LCMS method 2: 99.9% purity at 215 nm [M+H]⁺=790.3.

¹H NMR (400 MHz, DMSO-d6) δ ppm 0.88-1.03 (m, 2H), 1.19-1.25 (m, 1H), 1.26-1.40 (m, 2H), 1.40-1.54 (m, 3H), 1.55-1.76 (m, 5H), 1.78-1.90 (m, 4H), 1.94-2.07 (m, 3H), 2.11-2.22 (m, 2H), 2.43-2.49 (m, 2H), 2.53-2.64 (m, 2H), 2.81-2.96 (m, 1H), 3.44 (m, 4H), 3.61-3.79 (m, 2H), 5.07 (dd, J=12.9, 5.3 Hz, 1H), 6.02 (s, 1H), 7.25 (d, J=8.7 Hz, 1H), 7.33 (s, 1H), 7.56 (dd, J=8.9, 2.0 Hz, 1H), 7.68 (d, J=8.4 Hz, 1H), 7.93 (d, J=8.8 Hz, 1H), 8.01 (d, J=7.7 Hz, 1H), 8.35 (d, J=6.0 Hz, 1H), 8.39 (s, 1H), 8.70 (d, J=1.7 Hz, 1H), 9.14 (s, 1H), 9.24 (s, 1H), 11.08 (s, 1H).

Table 31 summarizes the compounds prepared using General Procedure X-2.

TABLE 31
Final Compounds Prepared via General Procedure X-2

| Compound | TMB | CBM | | |
|---|---|---|---|---|
| No. | Portion | Portion | Structure | Characterization |
| P-3 | A1 | C2 | 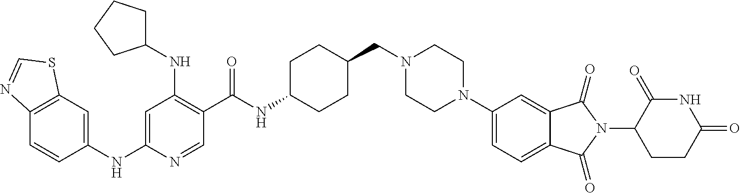 [SMILES: O=C1CCC(N2C(=O)c3ccc(N4CCN(C[C@H]5CC[C@H](NC(=O)c6cnc(Nc7ccc8ncsc8c7)cc6NC6CCCC6)CC5)CC4)cc3C2=O)C(=O)N1] | 62% yield as a yellow solid. LCMS method 2: retention time: 2.165 min, 99.9% purity at 215 nm, [M − HCOOH + H]⁺ = 790.3; [M − HCOOH + +2H ]²⁺ = 395.7 ¹H NMR (400 MHz, DMSO-d6) δ ppm 0.88-1.02 (m, 2H), 1.07-1.20 (m, 1H), 1.26-1.40 (m, 2H), 1.41-1.54 (m, 3H), 1.56-1.75 (m, 5H), 1.78-1.91 (m, 4H), 1.95-2.07 (m, 3H), 2.11-2.21 (m, 2H), 2.45-2.48 (m, 2H), 2.54-2.63 (m, 2H), 2.82-2.95 (m, 1H), 3.40-3.48 (m, 4H), 3.63-3.77 (m, 2H), 5.07 (dd, J = 13.0, 5.4 Hz, 1H), 6.02 (s, 1H), 7.26 (d, J = 8.6 Hz, 1H), 7.34 (s, 1H), 7.56 (dd, J = 8.9, 2.1 Hz, 1H), 7.68 (d, J = 8.6 Hz, 1H), 7.93 (d, J = 8.9 Hz, 1H), 8.01 (d, J = 7.7 Hz, 1H), 8.14 (s, 1H), 8.35 (d, J = 6.0 Hz, 1H), 8.39 (s, 1H), 8.70 (d, J = 2.0 Hz, 1H), 9.14 (s, 1H), 9.24 (s, 1H), 11.08 (s, 1H). One proton was not apparent, two signals overlaps with solvent peak in ¹H NMR. |
| P-15 | A8 | C5 | 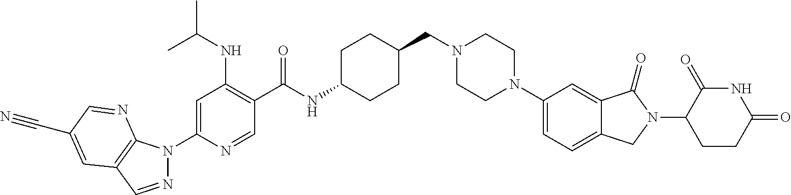 [SMILES: CC(C)Nc1cc(-n2ncc3cc(C#N)cnc32)ncc1C(=O)N[C@H]1CC[C@H](CN2CCN(c3ccc4c(c3)C(=O)N(C3CCC(=O)NC3=O)C4)CC2)CC1] | 58% yield as an off-white solid. LCMS method 2: retention time: 2.290 min, 99.0% purity at 215 nm, [M + 2H]²⁺ = 372.8; [M + H]⁺ = 744.3 ¹H NMR ( 400 MHz, DMSO-d6) δ ppm 0.90-1.05 (m, 2H), 1.24 (d, J = 6.2 Hz, 6H), 1.30-1.44 (m, 2H), 1.46-1.60 (m, 1H), 1.83-1.93 (m, 4H), 1.95-2.04 (m, 1H), 2.17 (d, J = 6.7 Hz, 2H), 2.30-2.44 (m, 1H), 2.46-2.54 (m, 2H), 2.55-2.65 (m, 1H), 2.84-2.97 (m, 1H), 3.13-3.25 (m, 4H), 3.26-3.49 (m, 2H), 3.67-3.84 (m, 2H), 4.17-4.25 (m, 1H), 4.29-4.37 (m, 1H), 5.10 (dd, J = 13.3, 5.0 Hz, 1H), 7.16 (s, 1H), 7.23-7.28 (m, 1H), 7.29 (s, 1H), 7.43 (d, J = 8.3 Hz, 1H), 8.40 (d, J = 7.5 Hz, 1H), 8.52 (d, J = 7.3 Hz, 1H), 8.60 (s, 1H), 8.64 (s, 1H), 9.01 (d, J = 1.9 Hz, 1H), 9.04 (d, J = 1.9 Hz, 1H), 10.97 (s, 1H). Two protons under the solvent peak by ¹H NMR. |
| P-17 | A7 | C5 | 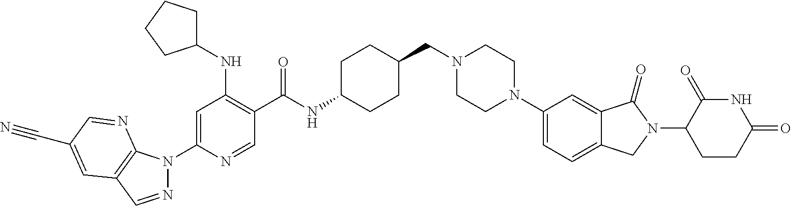 [SMILES: N#Cc1cnc2c(cnn2-c2cc(NC3CCCC3)c(C(=O)N[C@H]3CC[C@H](CN4CCN(c5ccc6c(c5)C(=O)N(C5CCC(=O)NC5=O)C6)CC4)CC3)cn2)c1] | 17% yield as an off-white solid. LCMS method 2: retention time: 2.428 min, 98.7% purity at 215 nm, [M + H]⁺ = 770.3, [M + 2H]²⁺ = 385.8 ¹H NMR (400 MHz, DMSO-d6) δ ppm 0.91-1.03 (m, 2H), 1.30-1.43 (m, 2H), 1.45-1.57 (m, 3H), 1.58-1.76 (m, 4H), 1.80-1.93 (m, 4H), 1.94-2.11 (m, 4H), 2.12-2.23 (m, 2H), 2.30-2.43 (m, 1H), 2.53-2.65 (m, 2H), 2.83-2.97 (m, 1H), 3.13-3.25 (m, 4H), 3.26-3.42 (m, 2H), 3.68-3.81 (m, 1H), 3.83-3.98 (m, 1H), 4.15-4.25 (m, 1H), 4.28-4.38 (m, 1H), 5.10 (dd, J = 13.0, 4.6 Hz, 1H), 7.13-7.19 (m, 1H), 7.26 (d, J = 8.2 Hz, 1H), 7.33 (s, 1H), 7.43 (d, J = 8.2 Hz, 1H), 8.40 (d, J = 7.1 Hz, 1H), 8.58-8.69 (m, 3H), 8.99-9.03 (m, 1H), 9.03-9.07 (m, 1H), 10.97 (s, 1H). Two signals overlaps with DMSO and two protons were under water by ¹H-NMR |
| P-18 | A7 | C2 | 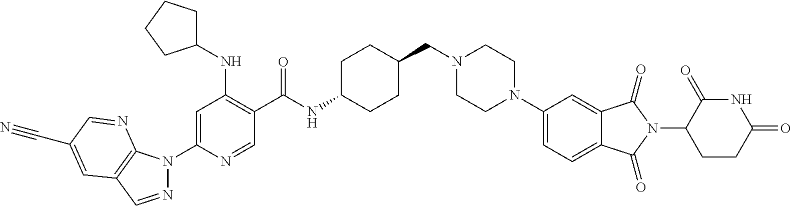 [SMILES: N#Cc1cnc2c(cnn2-c2cc(NC3CCCC3)c(C(=O)N[C@H]3CC[C@H](CN4CCN(c5ccc6c(c5)C(=O)N(C5CCC(=O)NC5=O)C6=O)CC4)CC3)cn2)c1] | 48% yield as a white solid. LCMS method 2: retention time: 2.506 min, 99.0% purity at 215 nm, [M + H]⁺ = 784.3, [M + 2H]²⁺ = 392.7 ¹H NMR (400 MHz, DMSO-d6) δ ppm 0.90-1.05 (m, 2H), 1.28-1.43 (m, 2H), 1.45-1.56 (m, 3H), 1.59-1.75 (m, 4H), 1.81-1.95 (m, 4H), 1.98-2.11 (m, 3H), 2.12-2.20 (m, 2H), 2.45-2.49 (m, 4H), 2.53-2.63 (m, 2H), 2.81-2.95 (m, 1H), 3.39-3.49 (m, 4H), 3.67-3.81 (m, 1H), 3.83-3.95 (m, 1H), 5.07 (dd, J = 12.9, 5.3 Hz, 1H), 7.25 (d, J = 8.6 Hz, 1H), 7.30-7.37 (m, 2H), 7.67 (d, J = 8.6 Hz, 1H), 8.40 (d, J = 7.6 Hz, 1H), 8.58-8.66 (m, 3H), 9.01 (d, J = 2.0 Hz, 1H), 9.05 (d, J = 1.8 Hz, 1H), 11.08 (s, 1H). Two signals partially overlap with solvent peak in ¹H NMR |
| P-24 | A7 | C5 | 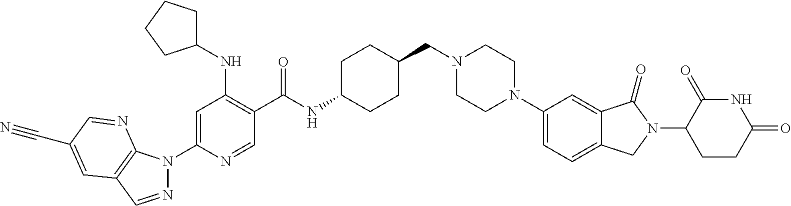 [SMILES: N#Cc1cnc2c(cnn2-c2cc(NC3CCCC3)c(C(=O)N[C@H]3CC[C@H](CN4CCN(c5ccc6c(c5)C(=O)N(C5CCC(=O)NC5=O)C6)CC4)CC3)cn2)c1] | 42% yield as a white solid. LCMS method 2: retention time: 2.676 min, 98.0% purity at 215 nm, [M + H]⁺ = 769.4, [M + 2H]²⁺ = 385.2 ¹H NMR (400 MHz, DMSO-d6) δ ppm 0.90-1.04 (m, 2H), 1.29-1.43 (m, 2H), 1.45-1.61 (m, 3H), 1.62-1.78 (m, 4H), 1.80-1.92 (m, 4H), 1.92-2.05 (m, 1H), 2.06-2.23 (m, 4H), 2.29-2.44 (m, 1H), 2.53-2.65 (m, 2H), 2.82-2.97 (m, 1H), 3.12-3.24 (m, 4H), 3.27-3.60 (m, 2H), 3.67-3.79 (m, 1H), 3.84-3.96 (m, 1H), 4.14-4.25 (m, 1H), 4.28-4.39 (m, 1H), 5.09 (dd, J = 13.3, 5.0 Hz, 1H), 6.88 (d, J = 3.8 Hz, 1H), 7.16 (s, 1H), 7.25 (d, J = 8.2 Hz, 1H), 7.42 (d, J = 8.4 Hz, 1H), 8.10-8.18 (m, 1H), 8.33 (d, J = 7.5 Hz, 1H), 8.53 (d, J = 3.8 Hz, 1H), 8.57 (s, 1H), 8.65-8.68 (m, 1H), 8.70 (d, J = 5.9 Hz, 1H), 8.82 (s, 1H), 10.97 (s, 1H). One proton was not apparent by ¹H NMR.. Two proton signals under the peak of water, and two signals overlap with DMSO by ¹H-NMR |
| P-29 | A7 | C14 | 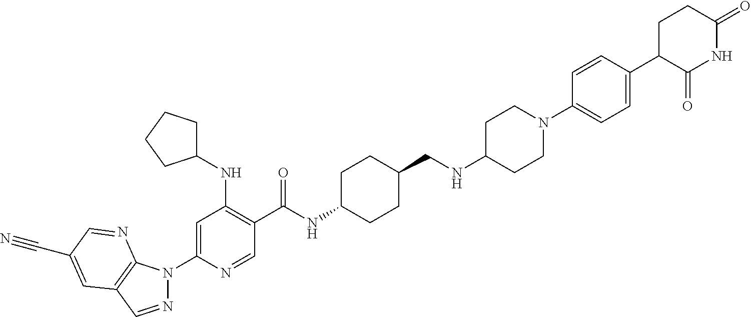 [SMILES: N#Cc1cnc2c(cnn2-c2cc(NC3CCCC3)c(C(=O)N[C@H]3CC[C@H](CNC4CCN(c5ccc(C6CCC(=O)NC6=O)cc5)CC4)CC3)cn2)c1] | 56% yield as a white solid. LCMS method 2: retention time: 2.416 min, 96.4% purity at 215 nm, [M + H]⁺ = 729.3, [M + 2H]²⁺ = 365.3 ¹H NMR (400 MHz, DMSO-d6) δ ppm 0.94-1.07 (m, 2H), 1.28-1.44 (m, 5H), 1.46-1.56 (m, 2H), 1.58-1.76 (m, 5H), 1.82-2.16 (m, 10H), 2.43 (m, 1H), 2.57-2.77 (m, 5H), 3.64 (m, J = 12.2 Hz, 2H), 3.69-3.78 (m, 2H), 3.85-3.94 (m, 1H), 6.89 (d, J = 8.6 Hz, 2H), 7.03 (d, J = 8.6 Hz, 2H), 7.33 (s, 1H), 8.26 (s, 1H), 8.37 (d, J = 7.6 Hz, 1H), 8.57-8.68 (m, 3H), 9.03 (d, J = 12.5 Hz, 2H), 10.76 (s, 1H). |
| P-31 | A7 | C15 | 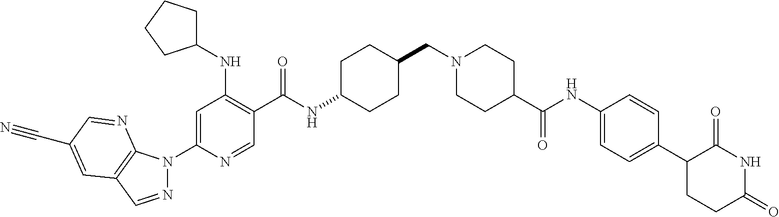 | 24% yield as a white solid. LCMS method 2: retention time: 2.349 min, 99.9% purity at 215 nm, [M + H]⁺ = 757.2, [M + 2H]²⁺ = 379.2 ¹H NMR (400 MHz, DMSO-d6) δ ppm 0.82-1.02 (m, 2H), 1.11-2.23 (m, 25H), 2.26-2.37 (m, 1H), 2.42-2.48 (m, 1H), 2.60-2.71 (m, 1H), 2.85-3.04 (m, 2H), 3.67-3.83 (m, 2H), 3.86-3.96 (m, 1H), 7.13 (d, J = 8.6 Hz, 2H), 7.33 (s, 1H), 7.55 (d, J = 8.3 Hz, 2H), 8.40 (d, J = 7.6 Hz, 1H), 8.56-8.68 (m, 3H), 9.02 (d, J = 1.5 Hz, 1H), 9.05 (d, J = 1.5 Hz, 1H), 9.84 (s, 1H), 10.80 (s, 1H). |
| P-32 | A7 | C7 | 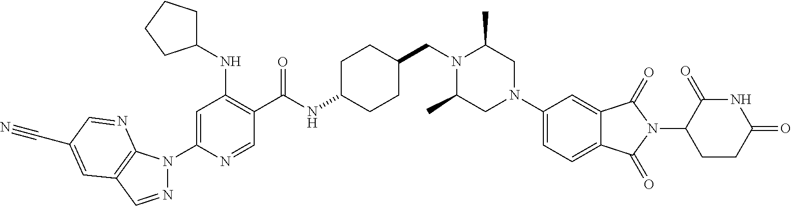 | 13% yield as a yellow solid. LCMS method 2: retention time: 2.503 min, 99.7% purity at 215 nm, [M + H]⁺ = 812.3, [M + 2H]²⁺ = 406.8 ¹H NMR (400 MHz, DMSO-d6) δ ppm 0.89-1.03 (m, 3H), 1.06-1.15 (m, 6H), 1.25-1.40 (m, 3H), 1.48-1.55 (m, 2H), 1.56-1.75 (m, 5H), 1.84-1.94 (m, 4H), 1.97-2.12 (m, 3H), 2.32-2.34 (m, 1H), 2.53-2.62 (m, 3H), 2.66-2.79 (m, 2H), 2.83-2.95 (m, 1H), 3.69-3.80 (m, 1H), 3.81-3.93 (m, 3H), 5.07 (q, J = 1.0 Hz, 1H), 7.20-7.26 (m, 1H), 7.31-7.37 (m, 2H), 7.64-7.69 (m, 1H), 8.19-8.25 (m, 1H), 8.33-8.40 (m, 1H), 8.58-8.68 (m, 3H), 9.00-9.08 (m, 2H). |
| P-36 | A7 | C5 | 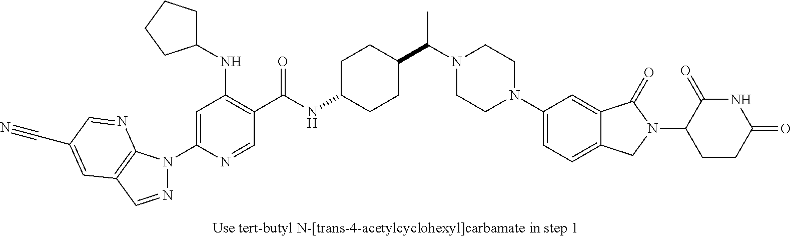 | 18 % yield as a yellow solid. LCMS method 2: retention time: 2.392 min, 95.6% purity at 215 nm, [M + H]⁺ = 784.3, [M + 2H]²⁺ = 392.7 ¹H NMR (400 MHz, DMSO-d6) δ ppm 0.90-1.08 (m, 5H), 1.27-1.41 (m, 3H), 1.46-1.56 (m, 2H), 1.57-1.75 (m, 4H), 1.78-2.11 (m, 6H), 2.11-2.20 (m, 1H), 2.23-2.44 (m, 2H), 2.44-2.48 (m, 1H), 2.54-2.63 (m, 1H), 2.65-2.74 (m, 2H), 2.85-2.97 (m, 1H), 3.12-3.25 (m, 4H), 3.67-3.78 (m, 1H), 3.85-3.95 (m, 1H), 4.21 (d, J = 17.1 Hz, 1H), 4.34 (d, J = 16.6 Hz, 1H), 5.09 (dd, J = 13.3, 5.3 Hz, 1H), 7.16 (d, J = 2.2 Hz, 1H), 7.25 (dd, J = 8.6, 2.2 Hz, 1H), 7.33 (s, 1H), 7.43 (d, J = 8.6 Hz, 1H), 8.38 (d, J = 7.6 Hz, 1H), 8.59-8.67 (m, 3H), 9.02 (d, J = 2.2 Hz, 1H), 9.05 (d, J = 2.2 Hz, 1H), 10.96 (s, 1H). One proton was under the solvent peak and one proton was not apparent by ¹H NMR. |
| P-37 | A7 | C17 | 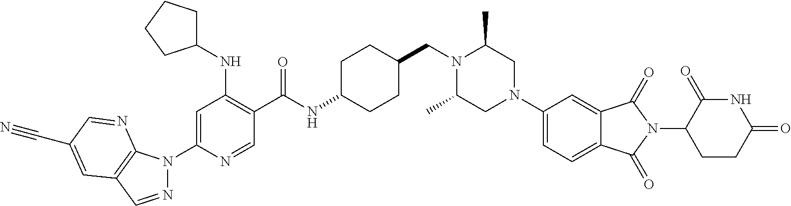 | 17% yield as a yellow solid. LCMS method 2: retention time: 2.471 min, 99.3% purity at 215 nm, [M + H]⁺ = 812.3, [M + 2H]²⁺ = 406.8 ¹H NMR (400 MHz, DMSO-d6) δ ppm 0.97-1.01 (m, 6H), 1.30-1.40 (m, 3H), 1.46-1.55 (m, 2H), 1.59-1.73 (m, 4H), 1.83-1.94 (m, 4H), 1.98-2.07 (m, 3H), 2.07-2.09 (m, 2H), 2.11-2.20 (m, 1H), 2.37-2.41 (m, 1H), 2.54-2.62 (m, 2H), 2.82-2.89 (m, 1H), 2.91-2.98 (m, 2H), 3.19-3.26 (m, 2H), 3.46-3.53 (m, 2H), 3.70-3.78 (m, 1H), 3.87-3.93 (m, 1H), 5.03-5.09 (m, 1H), 7.19-7.25 (m, 1H), 7.33 (s, 2H), 7.62-7.67 (m, 1H), 8.35-8.41 (m, 1H), 8.59-8.65 (m, 3H), 9.03 (dd, J = 14.5, 1.8 Hz, 2H), 11.06-11.09 (m, 1H). |
| P-38 | A7 | C18 | 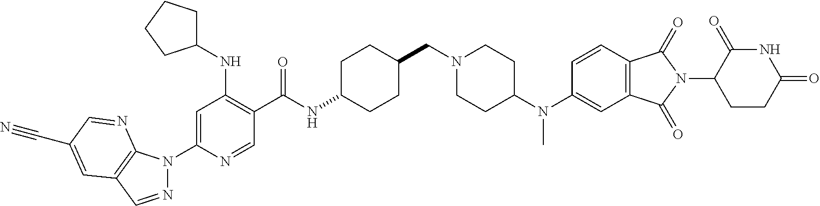 | 23% yield as a yellow solid. LCMS method 2: retention time: 2.453 min, 96.8% purity at 215 nm, [M + H]⁺ = 812.3, [M + 2H]²⁺ = 406.8 ¹H NMR (400 MHz, DMSO-d6) δ ppm 0.87-1.05 (m, 2H), 1.29-1.41 (m, 2H), 1.42-1.57 (m, 3H), 1.58-1.92 (m, 12H), 1.96-2.18 (m, 7H), 2.53-2.63 (m, 2H), 2.81-2.97 (m, 6H), 3.68-3.80 (m, 1H), 3.81-3.96 (m, 2H), 5.06 (dd, J = 12.8, 5.3 Hz, 1H), 7.09 (br d, J = 8.6 Hz, 1H), 7.17 (d, J = 1.5 Hz, 1H), 7.33 (s, 1H), 7.64 (d, J = 8.6 Hz, 1H), 8.39 (br d, J = 7.6 Hz, 1H), 8.57-8.68 (m, 3H), 9.01 (d, J = 2.0 Hz, 1H), 9.05 (d, J = 1.7 Hz, 1H), 11.06 (s, 1H). |
| P-39 | A7 | C19 | 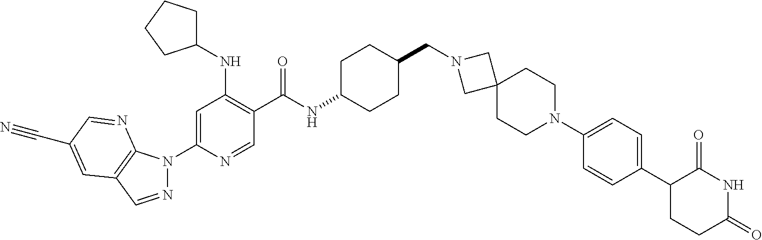 | 23% yield as a white solid. LCMS method 2: retention time: 2.381 min, 96.8% purity at 215 nm, [M − HCOOH + H]⁺ = 755.3; [M − HCOOH + 2H]²⁺ = 378.3 ¹H NMR (400 MHz, DMSO-d6) δ ppm 0.90-1.01 (m, 2H), 1.19-1.37 (m, 4H), 1.46-1.55 (m, 2H), 1.58-1.90 (m, 12H), 1.98-2.14 (m, 4H), 2.29 (d, J = 6.4 Hz, 2H), 2.41-2.46 (m, 1H), 2.57-2.64 (m, 1H), 2.97 (s, 4H), 3.04-3.09 (m, J = 5.1 Hz, 3H), 3.71 (dd, J = 10.9, 5.0 Hz, 2H), 3.86-3.93 (m, 1H), 6.88 (d, J = 8.6 Hz, 2H), 7.03 (d, J = 8.6 Hz, 2H), 7.33 (s, 1H), 8.22 (s, 1H), 8.36 (d, J = 7.8 Hz, 1H), 8.58-8.66 (m, 3H), 9.01 (d, J = 2.0 Hz, 1H), 9.05 (d, J = 2.0 Hz, 1H), 10.75 (s, 1H). One proton was not apparent by ¹H NMR. |
| P-41 | A14 | C2 | 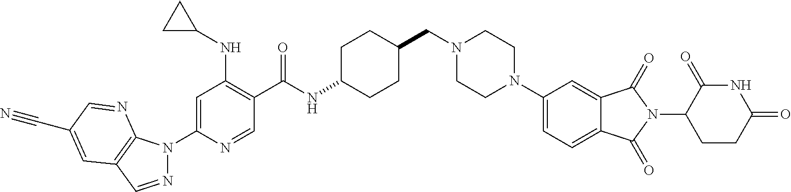 | 26% yield as a yellow solid. LCMS method 2: retention time: 2.555 min, 98.7% purity at 215 nm, [M + H]⁺ = 755.2, [M + 2H]²⁺ = 378.2 ¹H NMR (400 MHz, DMSO-d6) δ ppm 0.55-0.63 (m, 2H), 0.89-1.04 (m, 4H), 1.28-1.43 (m, 2H), 1.45-1.58 (m, 1H), 1.81-1.92 (m, 4H), 1.98-2.06 (m, 1H), 2.13-2.20 (m, 2H), 2.53-2.63 (m, 4H), 2.82-2.94 (m, 1H), 3.39-3.49 (m, 4H), 3.67-3.78 (m, 1H), 5.07 (dd, J = 13.0, 5.4 Hz, 1H), 6.90 (d, J = 3.9 Hz, 1H), 7.25 (d, J = 9.0 Hz, 1H), 7.33 (s, 1H), 7.68 (d, J = 8.3 Hz, 1H), 8.35 (d, J = 8.1 Hz, 1H), 8.53-8.59 (m, 3H), 8.62 (s, 1H), 8.68 (d, J = 2.0 Hz, 1H), 8.83 (d, J = 1.7 Hz, 1H), 11.07 (s, 1H). Three protons were obscured by water and DMSO in the ¹H NMR. |
| P-42 | A14 | C6 | 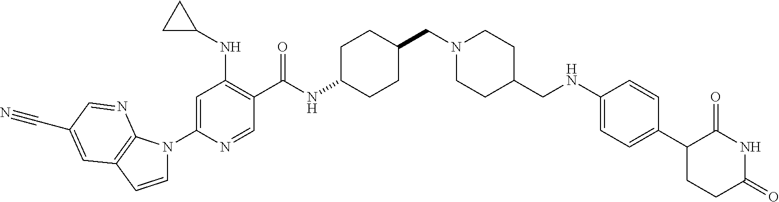 | 38% yield as a white solid. LCMS method 2: retention time: 2.545 min, 99.9% purity at 215 nm, [M + H]⁺ = 714.3, [M + 2H]²⁺ = 357.7 ¹H NMR (400 MHz, DMSO-d6) δ ppm 0.55-0.62 (m, 2H), 0.85-0.99 (m, 4H), 1.13-1.25 (m, 2H), 1.25-1.38 (m, 2H), 1.39-1.57 (m, 2H), 1.69-1.90 (m, 8H), 1.95-2.13 (m, 4H), 2.40-2.47 (m, 1H), 2.55-2.66 (m, 2H), 2.79-2.90 (m, 4H), 3.62 (dd, J = 10.3, 5.1 Hz, 1H), 3.66-3.76 (m, 1H), 5.54-5.61 (m, 1H), 6.51 (d, J = 8.3 Hz, 2H), 6.84-6.93 (m, 3H), 8.33 (d, J = 7.6 Hz, 1H), 8.54-8.59 (m, 3H), 8.61 (s, 1H), 8.66-8.70 (m, 1H), 8.83 (d, J = 1.7 Hz, 1H), 10.72 (s, 1H). |
| P-43 | A7 | C21 | 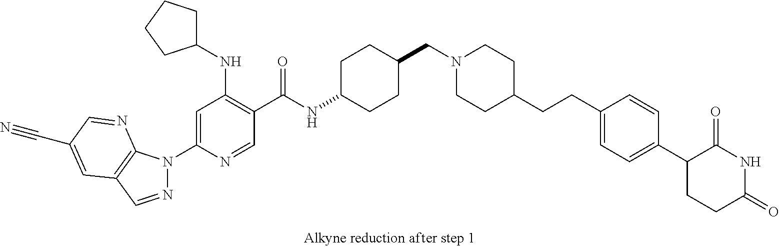 | 31% yield as a white solid. LCMS method 2: retention time: 2.525 min, 99.1% purity at 215 nm, [M + H]⁺ = 742.3, [M + 2H]²⁺ = 371.8 ¹H NMR (400 MHz, DMSO-d6) δ ppm 0.86-0.97 (m, 2H), 1.14-1.25 (m, 3H), 1.27-1.38 (m, 3H), 1.42-1.55 (m, 5H), 1.60-1.74 (m, 6H), 1.77-1.91 (m, 6H), 2.02-2.10 (m, 4H), 2.13-2.22 (m, 1H), 2.43-2.46 (m, 1H), 2.54-2.67 (m, 3H), 2.78-2.84 (m, 2H), 3.67-3.76 (m, 1H), 3.75-3.85 (m, 1H), 3.85-3.94 (m, 1H), 7.09-7.18 (m, 4H), 7.33 (s, 1H), 8.38 (d, J = 8.3 Hz, 1H), 8.59-8.67 (m, 3H), 9.01 (d, J = 2.0 Hz, 1H), 9.05 (d, J = 2.2 Hz, 1H), 10.80 (s, 1H). |
| P-46 | A7 | C23 | 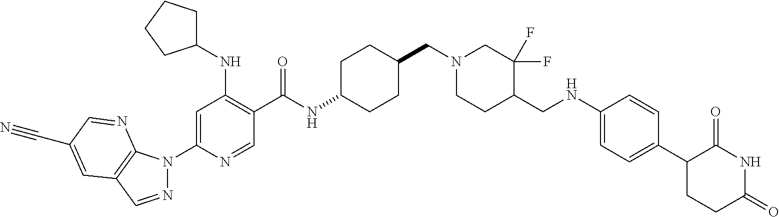 | 15% yield as a white solid. LCMS method 2: retention time: 2.660 min, 97.8% purity at 215 nm, [M + H]⁺ = 793.2, [M + 2H]²⁺ = 397.3 ¹H NMR (400 MHz, DMSO-d6) δ ppm 0.89-1.03 (m, 2H), 1.28-1.45 (m, 3H), 1.46-1.56 (m, 3H), 1.58-1.66 (m, 2H), 1.67-1.77 (m, 2H), 1.79-1.92 (m, 5H), 1.97-2.11 (m, 4H), 2.12-2.28 (m, 4H), 2.36-2.44 (m, 1H), 2.59-2.67 (m, 1H), 2.78-2.88 (m, 1H), 2.89-3.02 (m, 1H), 3.03-3.14 (m, 1H), 3.67-3.77 (m, 1H), 3.77-3.84 (m, 1H), 3.86-3.95 (m, 1H), 7.16 (d, J = 8.8 Hz, 2H), 7.33 (s, 1H), 7.55 (d, J = 8.6 Hz, 2H), 8.38-8.43 (d, J = 7.58 Hz, 1H), 8.60-8.65 (m, 3H), 9.01 (d, J = 2.0 Hz, 1H), 9.05 (d, J = 2.0 Hz, 1H), 10.04 (s, 1H), 10.80 (s, 1H). ¹⁹F NMR (377 MHz, DMSO-d6) δ ppm −107.54 (br d, J = 238.4 Hz, 1F),-97.61 (br d, J = 239.8 Hz, 1F). |
| P-53 | A15 | C2 | 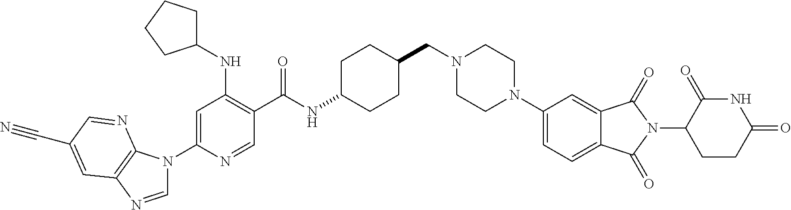 | 20% yield as a yellow solid. LCMS method 2: retention time: 2.595 min, 96.3% purity at 215 nm, [M + H]⁺ = 784.4, [M + 2H]²⁺ = 392.8 ¹H NMR (400 MHz, DMSO-d6) δ ppm 0.88-1.06 (m, 3H), 1.28-1.43 (m, 2H), 1.45-1.60 (m, 3H), 1.60-1.78 (m, 4H), 1.81-1.94 (m, 4H), 1.96-2.06 (m, 1H), 2.07-2.22 (m, J = 5.9 Hz, 4H), 2.52-2.63 (m, 3H), 2.82-2.94 (m, 1H), 3.44 (s, 4H), 3.68-3.80 (m, 1H), 3.87-3.99 (m, 1H), 5.01-5.13 (m, 1H), 7.26 (d, J = 7.6 Hz, 1H), 7.34 (s, 1H), 7.68 (d, J = 8.3 Hz, 1H), 7.92 (s, 1H), 8.41 (d, J = 6.8 Hz, 1H), 8.60 (s, 1H), 8.79 (d, J = 5.6 Hz, 1H), 8.87 (s, 1H), 8.97 (s, 1H), 9.37 (s, 1H), 11.07 (s, 1H). Two protons were not apparent by ¹H NMR. |
| P-58 | A7 | C27 | 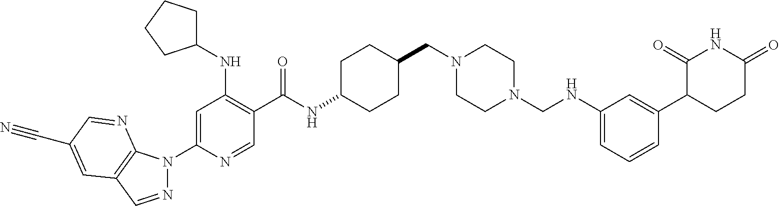 | 19% yield as an off-white solid. LCMS method 2: retention time: 2.419 min, 94.9% purity at 215 nm, [M + H]⁺ = 743.5, [M + 2H]²⁺ = 372.3 ¹H NMR (400 MHz, DMSO-d6) δ ppm 0.88-1.04 (m, 2H), 1.19-1.28 (m, 2H), 1.30-1.39 (m, 2H), 1.46-1.57 (m, 4H), 1.61-1.91 (m, 11H), 1.95-2.13 (m, 6H), 2.16-2.26 (m, 2H), 2.57-2.68 (m, 1H), 2.82-3.00 (m, 4H), 3.64-3.78 (m, 2H), 3.85-3.95 (m, 1H), 5.63 (s, 1H), 6.29-6.50 (m, 3H), 7.00 (t, J = 7.5 Hz, 1H), 7.33 (s, 1H), 8.36-8.43 (m, 1H), 8.56-8.70 (m, 3H), 9.03 (d, J = 14.0 Hz, 2H), 10.79 (s, 1H). |
| P-60 | A7 | C29 | 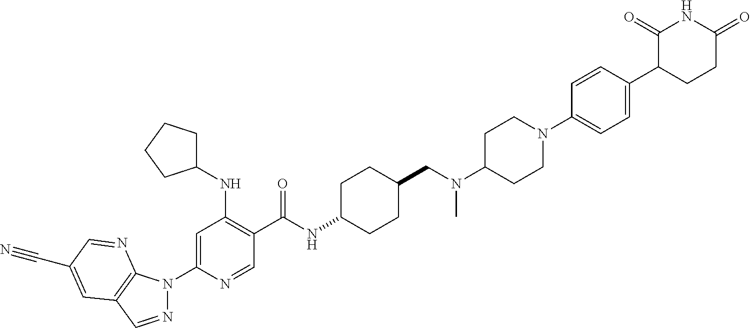 | 37% yield as a light yellow solid. LCMS method 2: retention time: 2.411 min, 98.8% purity at 215 nm, [M + H]⁺ = 743.5, [M + 2H]²⁺ = 372.2 ¹H NMR (400 MHz, DMSO-d6) δ ppm 0.85-0.99 (m, 2H), 1.27-1.42 (m, 3H), 1.45-1.57 (m, 4H), 1.59-1.78 (m, 6H), 1.83-1.88 (m, 4H), 1.97-2.15 (m, 4H), 2.20 (s, 3H), 2.21-2.25 (m, 2H), 2.41-2.46 (m, 1H), 2.57-2.69 (m, 4H), 3.67-3.76 (m, 4H), 3.86-3.93 (m, 1H), 6.89 (d, J = 8.6 Hz, 2H), 7.03 (d, J = 8.6 Hz, 2H), 7.33 (s, 1H), 8.37 (d, J = 7.6 Hz, 1H), 8.58-8.66 (m, 3H), 9.01 (d, J = 1.7 Hz, 1H), 9.05 (s, 1H), 10.76 (s, 1H). |
| P-69 | A7 | C33 | 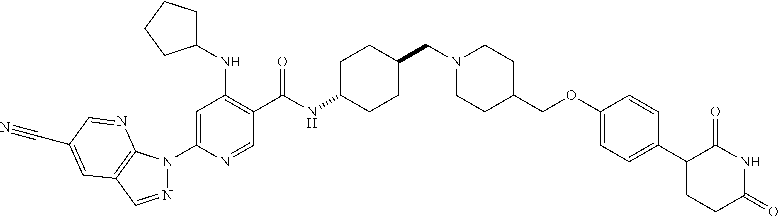 | 21% yield as a white solid. LCMS method 2: retention time: 2.469 min, 99.0% purity at 215 nm, [M + H]⁺ = 744.4, [M + 2H]²⁺ = 372.8 ¹H NMR (400 MHz, DMSO-d6) δ ppm 0.87-1.01 (m, 2H), 1.22-1.41 (m, 4H), 1.43-1.56 (m, 3H), 1.58-1.78 (m, 7H), 1.80-1.93 (m, 6H), 1.96-2.19 (m, 6H), 2.42-2.48 (m, 1H), 2.59-2.70 (m, 1H), 2.86 (br d, J = 9.9 Hz, 2H), 3.68-3.84 (m, 4H), 3.86-3.94 (m, 1H), 6.88 (d, J = 8.7 Hz, 2H), 7.11 (d, J = 8.6 Hz, 2H), 7.33 (s, 1H), 8.39 (br d, J = 7.5 Hz, 1H), 8.56-8.68 (m, 3H), 9.01 (d, J = 1.8 Hz, 1H), 9.05 (d, J = 1.8 Hz, 1H), 10.79 (s, 1H). |
| P-127 | A10 | C13 | 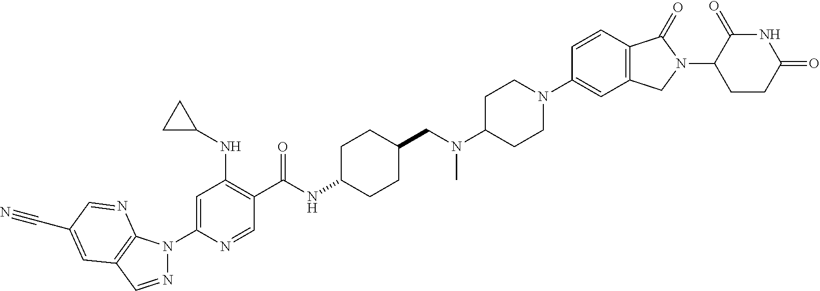 | 26% yield as a white solid. LCMS method 2: retention time: 2.223 min, 96.6% purity at 215 nm, [M + H]⁺ = 770.3, [M + 2H]²⁺ = 385.8 ¹H NMR (400 MHz, DMSO-d6) δ ppm 0.53-0.59 (m, 2H), 0.83-1.15 (m, 5H), 1.24-1.41 (m, 3H), 1.42-1.55 (m, 2H), 1.70-1.99 (m, 7H), 2.14-2.23 (m, 5H), 2.34-2.45 (m, 1H), 2.55-2.62 (m, 2H), 2.79-2.97 (m, 3H), 3.64-3.76 (m, 1H), 3.93 (br d, J = 12.5 Hz, 2H), 4.18-4.35 (m, 2H), 5.05 (dd, J = 13.2, 5.1 Hz, 1H), 7.02-7.07 (m, 2H), 7.50 (d, J = 8.6 Hz, 1H), 7.67 (s, 1H), 8.39 (d, J = 7.8 Hz, 1H), 8.54 (s, 1H), 8.60 (s, 1H), 8.66 (s, 1H), 9.03 (d, J = 2.0 Hz, 1H), 9.06 (d, J = 2.0 Hz, 1H), 10.94 (s, 1H). |
| P130 | A10 | C47 | 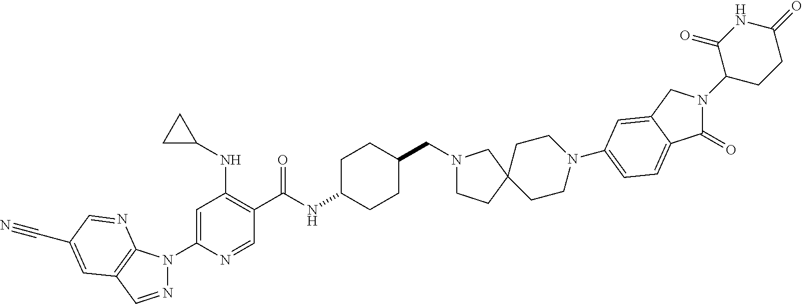 | 25% yield as a white solid. LCMS method 2: retention time: 2.245 min, 99.7% purity at 215 nm, [M + H]⁺ = 796.4, [M + 2H]²⁺ = 398.8 ¹H NMR (400 MHz, DMSO-d6) δ ppm 0.54-0.58 (m, 2H), 0.83-0.87 (m, 2H), 0.90-1.00 (m, 2H), 1.28-1.42 (m, 3H), 1.55-1.66 (m, 6H), 1.81-2.00 (m, 6H), 2.21 (d, J = 6.8 Hz, 2H), 2.30-2.42 (m, 4H), 2.54-2.61 (m, 3H), 2.85-2.95 (m, 1H), 3.66-3.77 (m, 1H), 4.17-4.22 (m, 1H), 4.28-4.34 (m, 1H), 5.04 (dd, J = 13.2, 5.1 Hz, 1H), 7.03-7.08 (m, 2H), 7.49 (d, J = 8.6 Hz, 1H), 7.67 (s, 1H), 8.40 (d, J = 7.6 Hz, 1H), 8.53 (s, 1H), 8.59 (s, 1H), 8.65 (s, 1H), 9.02 (d, J = 2.0 Hz, 1H), 9.06 (d, J = 2.0 Hz, 1H), 10.94 (s, 1H). Three protons were not apparent by ¹H NMR. |

### General Procedure X-3

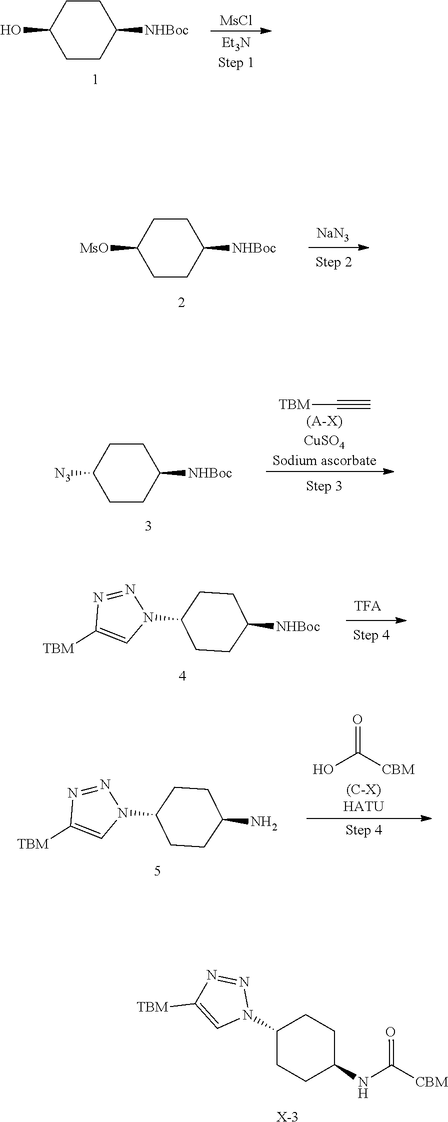

Step 1. Preparation of 4-((tert-butoxycarbonyl)amino)cyclohexyl methanesulfonate (2): In a round-bottom flask were added tert-butyl N-(4-hydroxycyclohexyl)carbamate 1 (1 equiv.) and DCM (0.46M), then the flask was cooled to 0° C. before methanesulfonyl chloride (1.1 equiv) and Et₃N (1.2 equiv.) were added, the last dropwise. After LCMS showed full conversion, the mixture was partitioned between EtOAc and water. The organic phase was washed once with water and once with 2N HCl, dried over magnesium sulfate, filtered and evaporated under reduced pressure to afford the product 2.

Step 2. Preparation of tert-butyl ((1r,4r)-4-azidocyclohexyl)carbamate (3): In a round-bottom flask were added [4-(tert-butoxycarbonylamino)cyclohexyl]methanesulfonate 2 (1 equiv.) and sodium azide (2 equiv.), in DMF (0.17 M) were heated to 70° C. overnight. The LCMS showed full conversion. The mixture was partitioned between MTBE and water. The organic phase was washed 1× with water and 1× with sat. aq. NaHCO₃, then dried over magnesium sulfate, filtered and evaporated under reduced pressure and concentrated under reduced pressure to give the desired product 3.

Step 3. Preparation of 4 via Click reaction: tert-butyl (4-azidocyclohexyl)carbamate 3 (1.25 equiv.), TBM A-X (1 equiv.), CuSO₄ (0.22 equiv.) and sodium ascorbate (48.24 mg, 0.2400 mmol) were charged in a small reaction vial and solubilized in Methanol (0.3 M):Water (0.3 M):THF (0.3 M). The reaction was stirred at RT for 1 h. After LCMS showed full conversion, the crude solution was purified to afford 4.

Step 4. Preparation of 5 via TFA deprotection: 4 (1 equiv.) was solubilized in DCM (0.1 M) after TFA (20 equiv.) was added. The reaction was stirred at RT overnight. LCMS showed full conversion. The crude was concentrated to afford 5.

Step 5. Preparation of final product X-3: To a solution of DIPEA (10 equiv.) and HATU (1.05 equiv.) in DMF (1.2361 mL), 5 (1 equiv.) was added at RT. After 5 min CBM C-X (1.2 equiv.) was added. The reaction was stirred at RT for 0.5 h. LCMS showed full conversion. The crude solution was purified to afford the desired product X-3.

### Example S42. N-((1r,4r)-4-(4-(6-(5-cyano-1H-pyrazolo[3,4-b]pyridin-1-yl)-4-(cyclopentylamino)pyridin-3-yl)-1H-1,2,3-triazol-1-yl)cyclohexyl)-1-(2-(2,6-dioxopiperidin-3-yl)-3-oxoisoindolin-5-yl)piperidine-4-carboxamide (P-16)

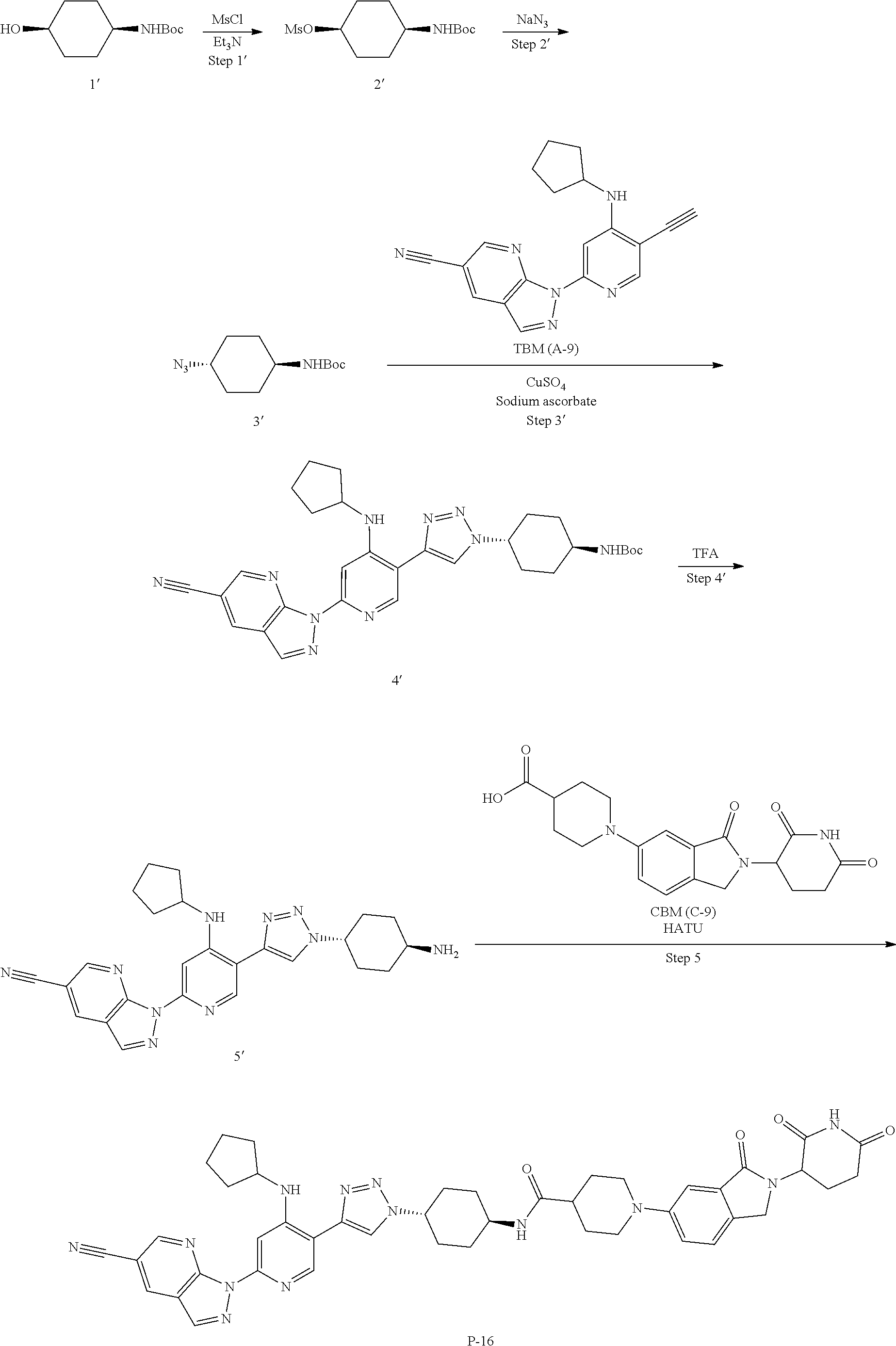

Step 1′. Preparation of 4-((tert-butoxycarbonyl)amino)cyclohexyl methanesulfonate (2′): In a 50 mL round-bottom flask were added tert-butyl N-(4-hydroxycyclohexyl)carbamate 1′ (0.65 g, 3.02 mmol, 1 equiv.) and DCM (6.5 mL, 0.46M), then the flask was cooled to 0° C. before methanesulfonyl chloride (0.26 mL, 3.32 mmol, 1.1 equiv) and Et₃N (0.5 mL, 3.62 mmol, 1.2 equiv.) were added, the last dropwise. After 2 h. LCMS showed full conversion. The mixture was partitioned between EtOAc and water. The organic phase was washed once with water and once with 2N HCl, dried over magnesium sulfate, filtered and evaporated under reduced pressure to afford the product 2′ in 94.5% yield. The crude was used directly in the next step.

LCMS condition 1: 92.5% purity at 215 nm, retention time, 1.630 min [M+H−Boc]⁺=194.0.

Step 2′. Preparation of tert-butyl ((1r,4r)-4-azidocyclohexyl)carbamate (3′): In a 100 mL round-bottom flask were added [4-(tert-butoxycarbonylamino)cyclohexyl]methanesulfonate 2′ (837. mg, 2.85 mmol, 1 equiv.) and sodium azide (370.94 mg, 5.71 mmol, 2 equiv.), in DMF (16.19 mL, 0.17 M) were heated to 70° C. overnight. The LCMS (showed full conversion. The mixture was partitioned between MTBE and water. The organic phase was washed 1× with water and 1× with sat. aq. NaHCO₃, then dried over magnesium sulfate, filtered and evaporated under reduced pressure and concentrated under reduced pressure to gave 0.69 g of desired product 3′ as a light pink solid.

LCMs condition 1: 85.0% purity at 215 nm, retention time, 1.825 min [M+H−Boc]⁺=140.3.

Step 3′. Preparation of 4′ tert-butyl ((1r,4r)-4-(4-(6-(5-cyano-1H-pyrazolo[3,4-b]pyridin-1-yl)-4-(cyclopentylamino)pyridin-3-yl)-1H-1,2,3-triazol-1-yl)cyclohexyl)carbamate: tert-butyl N-(4-azidocyclohexyl)carbamate 3′ (64.57 mg, 1-[4-(cyclopentylamino)-5-ethynyl-2-pyridyl]pyrazolo[3,4-b]pyridine-5-carbonitrile TBM A-9 (60. mg, 0.1800 mmol), CuSO₄ (5.83 mg, 0.0400 mmol) and sodium ascorbate (48.24 mg, 0.2400 mmol) were charged in a small reaction vial and solubilized in Methanol (0.6091 mL):Water (0.6091 mL):THF (0.6091 mL). The reaction was stirred at RT for 1 h. After LCMS showed full conversion, the crude solution was purified on a 50 g C18 reverse-phase chromatography column (MeOH/0.1% FA aq., 4 CV 5%, 5% to 100% in 15 CV, 100% for 4 CV) to afford 4′ in 92.8% yield.

LCMS method 1: 95.5% purity at 215 nm, retention time: 1.74 min, [M+H]⁺=569.4.

Step 4′. Preparation of 5′ 1-(5-(1-((1r,4r)-4-aminocyclohexyl)-1H-1,2,3-triazol-4-yl)-4-(cyclopentylamino)pyridin-2-yl)-1H-pyrazolo[3,4-b]pyridine-5-carbonitrile: 4′ (101. mg, 0.1800 mmol) was solubilized in DCM (1.7761 mL) after TFA (0.29 mL, 3.55 mmol) was added. The reaction was stirred at RT overnight. LCMS showed full conversion. The crude was evaporated with MeCN/PhMe (4×) to afford 5′.

LCMS method 1: 99.0% purity at 215 nm, retention time: 1.289 min, [M+H]⁺=470.4.

Step 5′. Preparation of final product P-16 N-((1r,4r)-4-(4-(6-(5-cyano-1H-pyrazolo[3,4-b]pyridin-1-yl)-4-(cyclopentylamino)pyridin-3-yl)-1H-1,2,3-triazol-1-yl)cyclohexyl)-1-(2-(2,6-dioxopiperidin-3-yl)-3-oxoisoindolin-5-yl)piperidine-4-carboxamide: To a solution of DIPEA (0.32 mL, 1.85 mmol) and HATU (70.5 mg, in DMF (1.2361 mL), 5′ (104. mg, 0.1800 mmol) was added at RT. After 5 min 1-[2-(2,6-dioxo-3-piperidyl)-3-oxo-isoindolin-5-yl]piperidine-4-carboxylic acid; 2,2,2-trifluoroacetic acid CBM C-9 (60. mg, 0.1200 mmol) was added. The reaction was stirred at RT for 0.5 h. LCMS showed full conversion. The crude solution was purified on a 50 g C18 reverse-phase chromatography column (MeCN/0.1% FA aq., 4 CV 5%, 5% to 100% in 15 CV) to afford the desired product 74% pure at 215 nm. A second purification on a 50 g C18 reverse-phase chromatography column (MeOH/0.1% FA aq., 4 CV 5%, 5% to 100% in 15 CV) afforded desired product P-16 in 99.9% pure at 215 nm.

LCMS method 2: retention time: 3.018 min, 99.9% purity at 215 nm, [M+H]⁺=822.3, [M+2H]²⁺=411.7.

¹H NMR (400 MHz, DMSO-d6) δ ppm 1.40-1.52 (m, 2H), 1.60-1.82 (m, 10H), 1.91-2.03 (m, 5H), 2.06-2.16 (m, 2H), 2.20-2.27 (m, 2H), 2.29-2.44 (m, 2H), 2.55-2.65 (m, 1H), 2.69-2.79 (m, 2H), 2.85-2.98 (m, 1H), 3.63-3.74 (m, 1H), 3.76-3.85 (m, 2H), 3.98-4.07 (m, 1H), 4.18-4.25 (m, 1H), 4.31-4.38 (m, 1H), 4.57-4.67 (m, 1H), 5.11 (dd, J=13.3, 5.0 Hz, 1H), 7.18 (d, J=1.7 Hz, 1H), 7.28 (dd, J=8.4, 1.8 Hz, 1H), 7.36 (s, 1H), 7.43 (d, J=8.3 Hz, 1H), 7.84 (d, J=7.6 Hz, 1H), 8.50 (d, J=6.1 Hz, 1H), 8.61 (s, 1H), 8.64 (s, 1H), 8.92 (s, 1H), 9.03 (d, J=2.0 Hz, 1H), 9.06 (d, J=2.0 Hz, 1H), 10.98 (s, 1H).

Table 32 summarizes the compounds prepared using General Procedure X-3.

TABLE 32
Final Compounds Prepared via General Procedure X-3

| Compound | TMB | CBM | | |
|---|---|---|---|---|
| No. | Portion | Portion | Structure | Characterization |
| P-5 | A3 | C3 | 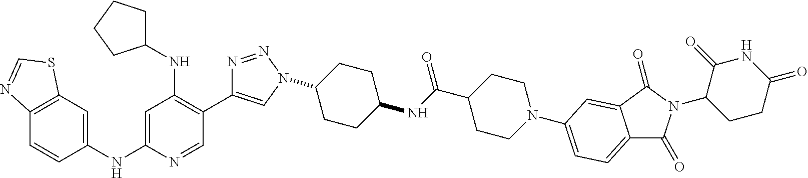 | 24% yield as a yellow solid. LCMS method 2: retention time: 2.845 min, 99.9% purity at 215 nm, [M + H]⁺ = 842.3, [M + 2H]²⁺ = 421.6 ¹H NMR (400 MHz, DMSO-d6) δ ppm 1.34-1.83 (m, 12H), 1.86-1.97 (m, 4H), 1.99-2.12 (m, 3H), 2.13-2.23 (m, 2H), 2.38-2.45 (m, 1H), 2.52-2.64 (m, 2H), 2.82-2.95 (m, 1H), 3.00 (br. t, J = 12.1 Hz, 2H), 3.60-3.71 (m, 1H), 3.79-3.89 (m, 1H), 4.08 (br. d, J = 13.0 Hz, 2H), 4.48-4.60 (m, 1H), 5.07 (dd, J = 12.8, 5.3 Hz, 1H), 6.15 (s, 1H), 7.22-7.29 (m, 1H), 7.32-7.37 (m, 1H), 7.53-7.60 (m, 1H), 7.67 (d, J = 8.6 Hz, 1H), 7.84 (d, J = 7.6 Hz, 1H), 7.92 (d, J = 8.8 Hz, 1H), 7.98 (d, J = 6.1 Hz, 1H), 8.28 (s, 1H), 8.62 (s, 1H), 8.69-8.74 (m, 1H), 9.03-9.20 (m, 2H), 11.08 (s, 1H). |
| P-16 | A9 | C9 | 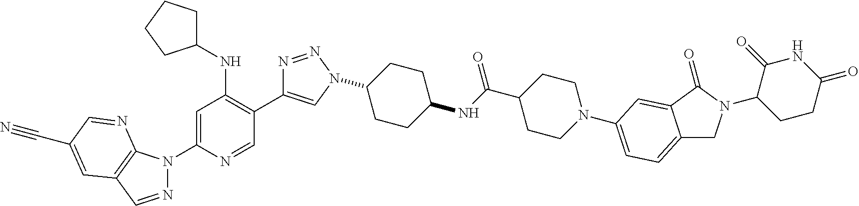 | 17% yield as a white solid. LCMS method 2: retention time: 3.018 min, 99.9% purity at 215 nm, [M + H]⁺ = 822.3, [M + 2H]²⁺ = 411.7 ¹H NMR (400 MHz, DMSO-d6) δ ppm 1.40-1.52 (m, 2H), 1.60-1.82 (m, 10H), 1.91-2.03 (m, 5H), 2.06-2.16 (m, 2H), 2.20-2.27 (m, 2H), 2.29-2.44 (m, 2H), 2.55-2.65 (m, 1H), 2.69-2.79 (m, 2H), 2.85-2.98 (m, 1H), 3.63-3.74 (m, 1H), 3.76-3.85 (m, 2H), 3.98-4.07 (m, 1H), 4.18-4.25 (m, 1H), 4.31-4.38 (m, 1H), 4.57-4.67 (m, 1H), 5.11 (dd, J = 13.3, 5.0 Hz, 1H), 7.18 (d, J = 1.7 Hz, 1H), 7.28 (dd, J = 8.4, 1.8 Hz, 1H), 7.36 (s, 1H), 7.43 (d, J = 8.3 Hz, 1H), 7.84 (d, J = 7.6 Hz, 1H), 8.50 (d, J = 6.1 Hz, 1H), 8.61 (s, 1H), 8.64 (s, 1H), 8.92 (s, 1H), 9.03 (d, J = 2.0 Hz, 1H), 9.06 (d, J = 2.0 Hz, 1H), 10.98 (s, 1H). Two signals overlap with the solvent signal in ¹H NMR. |
| P-19 | A9 | C9 | 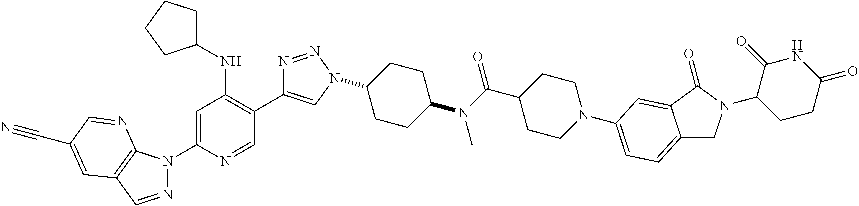 | 28% yield as a white solid. LCMS method 2: retention time: 3.154 min, 96.4% purity at 215 nm, [M + H]⁺ = 836.3, [M + 2H]²⁺ = 418.8 ¹H NMR (400 MHz, DMSO-d6) δ ppm 1.57-1.89 (m, 12H), 1.93-2.04 (m, 3H), 2.08-2.20 (m, 3H), 2.23-2.29 (m, 2H), 2.32-2.42 (m, 1H), 2.56-2.66 (m, 2H), 2.76 (s, 1H), 2.79-2.94 (m, 4H), 2.97 (s, 2H), 3.81 (br. d, J = 9.8 Hz, 2H), 3.97-4.06 (m, 1H), 4.18-4.26 (m, 1H), 4.30-4.38 (m, 1H), 4.41-4.52 (m, 1H), 4.59-4.70 (m, 1H), 5.10 (br. dd, J = 13.1, 5.0 Hz, 1H), 7.20 (br. s, 1H), 7.29 (br. d, J = 8.6 Hz, 1H), 7.44 (br. d, J = 8.3 Hz, 1H), 8.59-8.79 (m, 2H), 8.89-9.14 (m, 3H), 10.97 (s, 1H). Rotamers were observed by ¹H NMR. A signal overlap with the DMSO signal in ¹H NMR. Two protons were not apparent by ¹H NMR. |

### General Procedure X-4

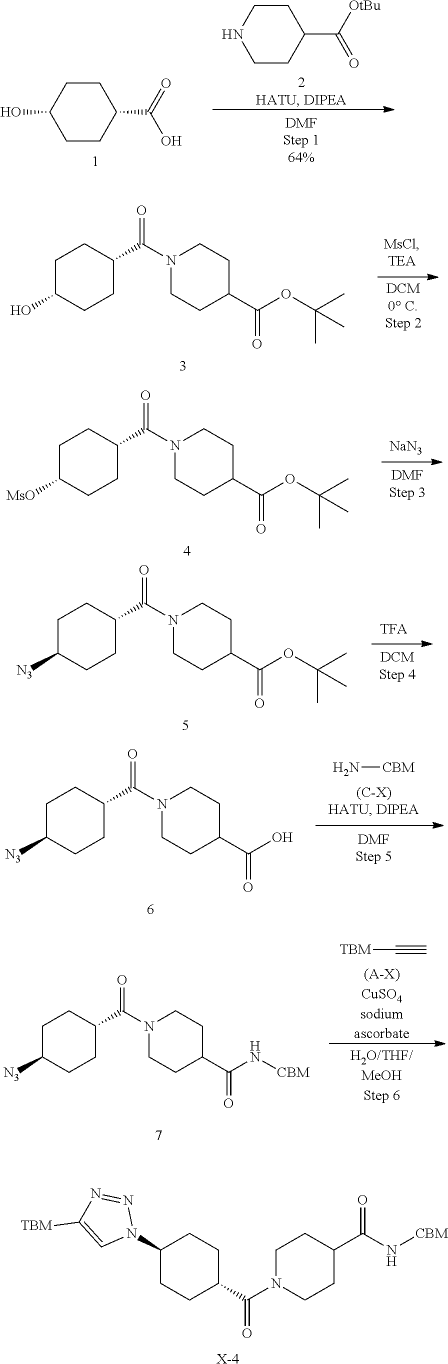

Step 1. Preparation of tert-butyl 1-(4-hydroxycyclohexanecarbonyl)piperidine-4-carboxylate (3): To a round bottom flask was added 4-hydroxycyclohexanecarboxylic acid 1 (1 eq.), tert-butyl piperidine-4-carboxylate;2,2,2-trifluoroacetic acid 2 (1 eq.) and DIPEA (3 eq.) in DMF (0.45 M). The reaction was stirred at room temperature for 5 minutes, then HATU (1.2 eq.) was added and the reaction stirred again at room temperature. After an overnight period, LCMS showed complete conversion into compound 3. The residue was purified by normal phase flash Fractions were combined and concentrated to give 3.

Step 2. Preparation of of tert-butyl 1-(4-methylsulfonyloxycyclohexanecarbonyl)piperidine-4-carboxylate (4): To a round bottom flask was added 2 (1 eq.) in DCM (0.1 M). The flask was then cooled to 0° C., then methanesulfonyl chloride (1.1 eq.) and triethylamine (1.2 eq.) were added. After TLC showed complete conversion of the starting material into the desired product. The mixture was partitioned between EtOAc and water. The organic phase was washed once with water and once with 1 N HCl, dried over magnesium sulfate, filtered and evaporated under reduced pressure. The crude 4 was used directly as is for the next reaction.

Step 3. Preparation of tert-butyl 1-[(2R,5R)-5-azido-2-ethyl-hexanoyl]piperidine-4-carboxylate (5): To a round bottom flask were added tert-butyl 1-(4-methylsulfonyloxycyclohexanecarbonyl)piperidine-4-carboxylate 4 (1 eq.) and NaN₃ (2 eq.) in DMF (0.66 M). The reaction mixture was then stirred at 70° C. After LCMS and TLC showed complete conversion of the starting material into compound 5. The mixture was partitioned between EtOAc and water. The organic phase was washed once with water and once with sat. NaHCO₃, then dried over magnesium sulfate, filtered and evaporated under reduced pressure. The residue was purified by normal phase flash chromatography to give product 5.

Step 4. Preparation of 1-(4-azidocyclohexanecarbonyl)piperidine-4-carboxylic acid (6): To a round bottom flask was added tert-butyl 1-[(2R,5R)-5-azido-2-ethyl-hexanoyl]piperidine-4-carboxylate 8 (1 eq.) in DCM (0.04 M), then TFA (86 eq.) was added to the mixture. The reaction was stirred at room temperature. After LCMS showed complete conversion into compound 6. The mixture was concentrated under reduced pressure and co-evaporated once with toluene and twice with acetonitrile to give 6.

Step 5. Preparation 7 via amide coupling: To a round bottom flask was added 1-(4-azidocyclohexanecarbonyl)piperidine-4-carboxylic acid 6 (1 eq.), CBM C-X (1 eq.) and DIPEA (10 eq.) in DMF (0.2 M). The reaction was stirred at room temperature for 5 minutes, then HATU (1.3 eq.) was added and the reaction stirred again at room temperature. After LCMS showed LCMS showed complete conversion into compound 7. The crude mixture was then purified by reverse phase FC. Fractions were combined and concentrated, affording 7.

Step 6. Preparation final product X-4: To a round bottom flask were added 8 (1 eq.), TBM A-X (1 eq.), sodium ascorbate (2 eq.) and CuSO₄ (0.2 eq.) in THF:H₂O:MeOH (1:1:1, 0.12 M) and the reaction mixture was stirred at room temperature. After LCMS showed complete conversion into the final product. The crude mixture was purified. Fractions were combined and concentrated, affording X-4.

### Example S43. 1-[4-[4-[6-(5-Cyanopyrazolo[3,4-b]pyridin-1-yl)-4-(cyclopentylamino)-3-pyridyl]triazol-1-yl]cyclohexanecarbonyl]-N-[4-(2,4-dioxohexahydropyrimidin-1-yl)phenyl]piperidine-4-carboxamide (P-45)

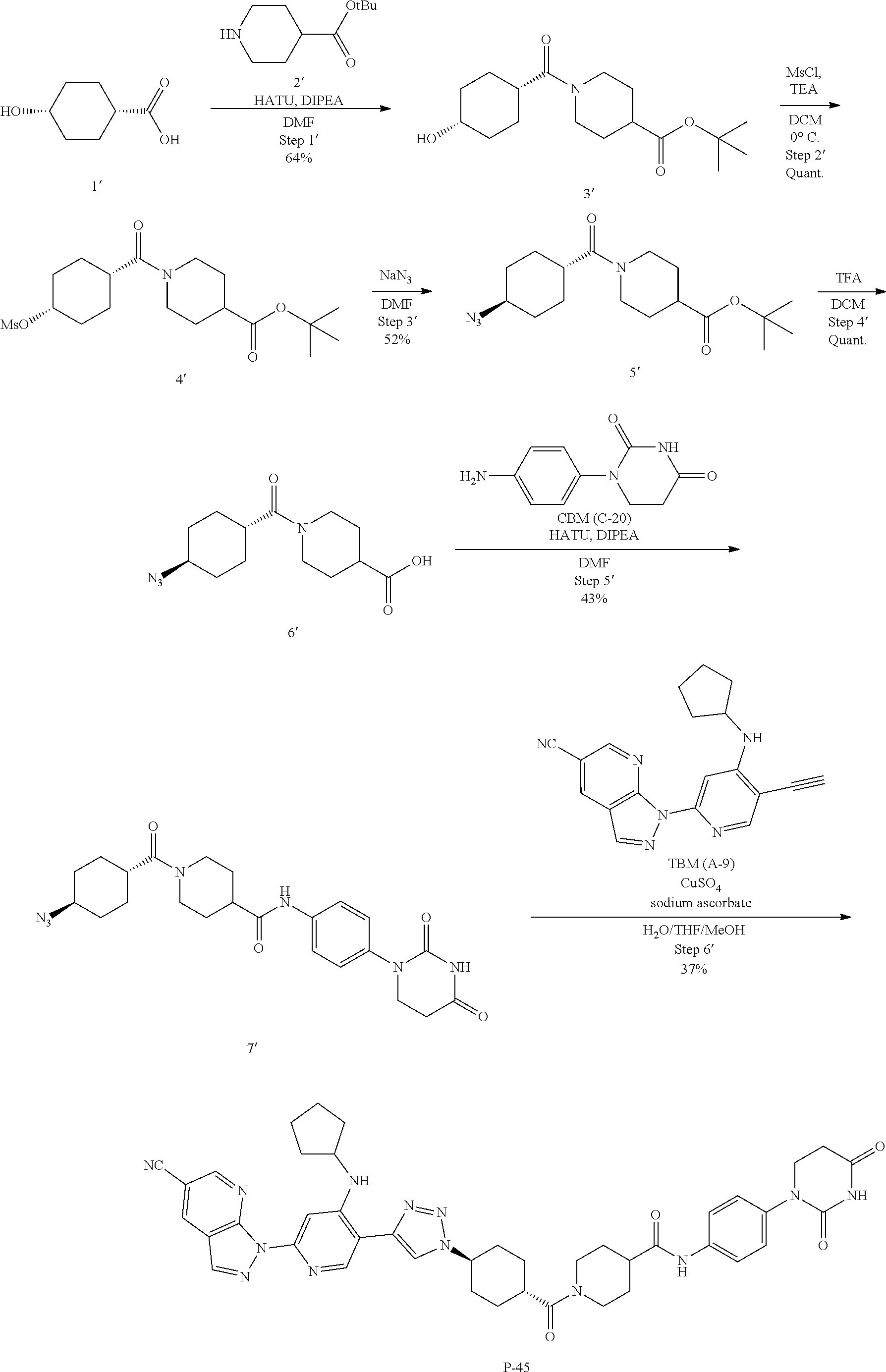

Step 1′. Preparation of tert-butyl 1-(4-hydroxycyclohexanecarbonyl)piperidine-4-carboxylate (3′): To a round bottom flask was added 4-hydroxycyclohexanecarboxylic acid 1′ (130.05 mg, 0.9 mmol, 1 eq.), tert-butyl piperidine-4-carboxylate;2,2,2-trifluoroacetic acid 2 (269.97 mg, 0.9 mmol, 1 eq.) and DIPEA (0.47 mL, 2.71 mmol, 3 eq.) in DMF (2 mL, 0.45 M). The reaction was stirred at room temperature for 5 minutes, then HATU (411.58 mg, 1.08 mmol, 1.2 eq.) was added and the reaction stirred again at room temperature. After an overnight period, LCMS showed complete conversion into compound 3. The residue was dry-packed and purified by normal phase flash chromatography (24 g silica column, elution: 5 to 100% EtOAc/Heptane over 10 CV, the product came out at 70% EtOAc). Fractions were combined and concentrated to give 3′ (282.8 mg, 64% yield) as a colorless oil.

LCMS method 1: 64.0% purity at 215 nm, [M+H]⁺=312.2.

¹H NMR (400 MHz, chloroform-d) δ ppm 1.45 (s, 9H), 1.49-1.68 (m, 6H), 1.83-2.00 (m, 6H), 2.38-2.56 (m, 2H), 2.73-2.80 (m, 1H), 3.11 (br s, 1H), 3.85 (br d, J=13.2 Hz, 1H), 4.01 (br s, 1H), 4.40 (br d, J=13.0 Hz, 1H). One proton was missing in the ¹H NMR spectrum.

Step 2′. Preparation of tert-butyl 1-(4-methylsulfonyloxycyclohexanecarbonyl)piperidine-4-carboxylate (4′): To a round bottom flask was added tert-butyl N-(4-hydroxycyclohexyl)carbamate 3′ (180.74 mg, 0.58 mmol, 1 eq.) in DCM (5.8 mL, 0.1 M). The flask was then cooled to 0° C., then methanesulfonyl chloride (49.4 μL, 0.64 mmol, 1.1 eq.) and triethylamine (97.1 μL, 0.70 mmol, 1.2 eq.) were added. After 2 h, TLC (Heptanes/EtOAc; 20/80) showed complete conversion of the starting material into the desired product (KMnO₄ revelator). The mixture was partitioned between EtOAc and water. The organic phase was washed once with water and once with 1 N HCl, dried over magnesium sulfate, filtered and evaporated under reduced pressure. The crude 4′ was used directly as is for the next reaction.

Step 3′. Preparation of tert-butyl 1-[(2R,5R)-5-azido-2-ethyl-hexanoyl]piperidine-4-carboxylate (5′): To a round bottom flask were added tert-butyl 1-(4-methylsulfonyloxycyclohexanecarbonyl)piperidine-4-carboxylate 4′ (324.2 mg, 0.83 mmol, 1 eq.) and NaN₃ (108.22 mg, 1.66 mmol, 2 eq.) in DMF (1.26 mL, 0.66 M). The reaction mixture was then stirred at 70° C. After an overnight period, LCMS and TLC (EtOAc/Heptane; 50/50) showed complete conversion of the starting material into compound 5 (KMnO₄). The mixture was partitioned between EtOAc and water. The organic phase was washed once with water and once with sat. NaHCO₃, then dried over magnesium sulfate, filtered and evaporated under reduced pressure. The residue was dry-packed and purified by normal phase flash chromatography (40 g silica column, elution: 5 to 100% EtOAc/Heptane over 15 CV). Fractions were combined and concentrated to give 5′ (157.8 mg, 56% yield) as a colorless oil.

LCMS method 1: 97.9% purity at 215 nm, [M+H]⁺=337.2.

¹H NMR (400 MHz, chloroform-d) δ ppm 1.32-1.44 (m, 2H), 1.47 (s, 9H), 1.58-1.69 (m, 4H), 1.78-1.98 (m, 4H), 2.07-2.17 (m, 2H), 2.42-2.53 (m, 2H), 2.76-2.89 (m, 1H), 3.14 (br t, J=12.1 Hz, 1H), 3.25-3.42 (m, 1H), 3.85 (br d, J=13.9 Hz, 1H), 4.41 (br d, J=13.0 Hz, 1H).

Step 4′. Preparation of 1-(4-azidocyclohexanecarbonyl)piperidine-4-carboxylic acid (6′): To a round bottom flask was added tert-butyl 1-[(2R,5R)-5-azido-2-ethyl-hexanoyl]piperidine-4-carboxylate 5′ (157.8 mg, 0.45 mmol, 1 eq.) in DCM (10 mL, 0.04 M), then TFA (3 mL, 38.67 mmol, 86 eq.) was added to the mixture. The reaction was stirred at room temperature. After 1 h, LCMS showed complete conversion into compound 6. The mixture was concentrated under reduced pressure and co-evaporated once with toluene and twice with acetonitrile to give 6′ (160 mg, quantitative yield).

¹H NMR (400 MHz, chloroform-d) δ ppm 1.24-1.46 (m, 2H), 1.52-1.89 (m, 6H), 1.97-2.22 (m, 4H), 2.37-2.57 (m, 1H), 2.57-2.71 (m, 1H), 2.85-3.04 (m, 1H), 3.13-3.42 (m, 2H), 3.80-4.00 (m, 1H), 4.37 (br d, J=9.8 Hz, 1H).

Step 5′. Preparation of 1-(4-azidocyclohexanecarbonyl)-N-[4-(2,4-dioxohexahydropyrimidin-1-yl)phenyl]piperidine-4-carboxamide (7′): To a round bottom flask was added 1-(4-azidocyclohexanecarbonyl)piperidine-4-carboxylic acid 6′ (109.29 mg, 0.39 mmol, 1 eq.), 1-(4-aminophenyl)hexahydropyrimidine-2,4-dione C-20 (80.01 mg, 0.39 mmol, 1 eq.) and DIPEA (0.68 mL, 3.9 mmol, 10 eq.) in DMF (2 mL, 0.2 M). The reaction was stirred at room temperature for 5 minutes, then HATU (192.71 mg, 0.51 mmol, 1.3 eq.) was added and the reaction stirred again at room temperature. After an overnight period, LCMS showed presence of compound 7 remaining, co-eluting with the desired product, so HATU (74.12 mg, 0.2 mmol, 0.5 eq.) was added. After 1 h, LCMS showed complete conversion into compound 8. The crude mixture was then purified by reverse phase FC purification (100 g C18 column, liquid deposit (DMSO), 5% MeCN/0.1% HCOOH over 5 CV, then 5 to 95% MeCN/0.1% HCOOH over 10 CV, the product came out at around 50 MeCN). Fractions were combined and concentrated, affording 7′ (79.3 mg, 43% yield) as a white solid.

LCMS method 1: 99.9% purity at 215 nm, [M+H]⁺=468.2.

¹H NMR (400 MHz, METHANOL-d4) δ ppm 1.40-1.51 (m, 2H), 1.55-1.67 (m, 3H), 1.79-2.01 (m, 5H), 2.03-2.12 (m, 2H), 2.64-2.78 (m, 3H), 2.83 (t, J=6.7 Hz, 2H), 3.13-3.25 (m, 2H), 3.87 (t, J=6.7 Hz, 2H), 4.16 (br d, J=13.0 Hz, 1H), 4.60 (d, J=13.0 Hz, 1H), 7.32 (d, J=8.8 Hz, 2H), 7.64 (br d, J=9.0 Hz, 2H).

Step 6′. Preparation of 1-[4-[4-[6-(5-Cyanopyrazolo[3,4-b]pyridin-1-yl)-4-(cyclopentylamino)-3-pyridyl]triazol-1-yl]cyclohexanecarbonyl]-N-[4-(2,4-dioxohexahydropyrimidin-1-yl)phenyl]piperidine-4-carboxamide (P-45): To a round bottom flask were added 1-(4-azidocyclohexanecarbonyl)-N-[4-(2,4-dioxohexahydropyrimidin-1-yl)phenyl]piperidine-4-carboxamide 8 (79.3 mg, 0.17 mmol, 1 eq.), 1-[4-(cyclopentylamino)-5-ethynyl-2-pyridyl]pyrazolo[3,4-b]pyrindine-5-carbonitrile 9 (55.7 mg, 0.17 mmol, 1 eq.), sodium ascorbate (67.17 mg, 0.34 mmol, 2 eq.) and CuSO₄ (5.41 mg, 0.03 mmol, 0.2 eq.) in THF:H₂O:MeOH (1:1:1, 0.12 M) and the reaction mixture was stirred at room temperature. After an overnight period, LCMS showed complete conversion into compound OMC-501-532393-003. The crude mixture was purified by reverse phase FC purification (50 g C18 column, liquid deposit (DMSO), 5% MeCN/0.1% HCOOH over 5 CV, then 5 to 100% MeCN/0.1% HCOOH over 15 CV, product came out around 40% MeCN). Fractions were combined and concentrated, affording P-45 (51.94 mg, 37% yield) as a light-yellow solid.

LCMS method 3: 97.4% purity at 215 nm, [M+H]⁺=796.3.

¹H NMR (400 MHz, DMSO-d6) δ ppm 1.40-1.50 (m, 1H), 1.59-1.75 (m, 9H), 1.83-1.98 (m, 6H), 2.07-2.28 (m, 4H), 2.61-2.77 (m, 5H), 3.05-3.18 (m, 1H), 3.70-3.76 (m, 2H), 3.82-3.97 (m, 1H), 4.01-4.14 (m, 1H), 4.40-4.51 (m, 1H), 4.54-4.66 (m, 1H), 7.24 (d, J=7.8 Hz, 2H), 7.60 (d, J=7.1 Hz, 2H), 7.66-9.94 (m, 7H), 9.99 (s, 1H), 10.32 (s, 1H).

Table 33 summarizes the compounds prepared using General Procedure X-4.

TABLE 33
Final Compounds Prepared via General Procedure X-4

| Compound | TMB | CBM | | |
|---|---|---|---|---|
| No. | Portion | Portion | Structure | Characterization |
| P-9 | A3 | C2 | 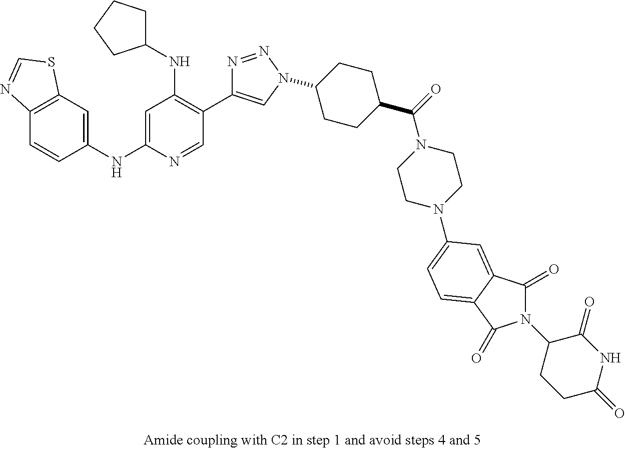 | 33% yield as a yellow solid. LCMS method 2: retention time: 2.782 min, 96.5% purity at 215 nm, [M + H]⁺ = 828.3, [M + 2H]²⁺ = 414.7 ¹H NMR (400 MHz, DMSO-d6) δ ppm 1.52-1.79 (m, 8H), 1.87-1.99 (m, 4H), 2.00-2.12 (m, 3H), 2.18-2.28 (m, 2H), 2.54-2.63 (m, 2H), 2.77-2.95 (m, 2H), 3.45-3.52 (m, 2H), 3.52-3.59 (m, 2H), 3.62-3.68 (m, 2H), 3.70-3.77 (m, 2H), 3.85-3.94 (m, 1H), 4.53-4.65 (m, 1H), 5.08 (dd, J = 12.7, 4.6 Hz, 1H), 6.19 (s, 1H), 7.27 (dd, J = 8.8, 1.7 Hz, 1H), 7.37 (br. s, 1H), 7.54 (d, J = 8.3 Hz, 1H), 7.69-7.75 (m, 1H), 7.96-8.09 (m, 1H), 8.17-8.28 (m, 1H), 8.59-8.85 (m, 1H), 9.18-9.33 (m, 1H), 11.08 (s, 1H). Three protons were not apparent by ¹H NMR. |
| P-27 | A9 | C13 | 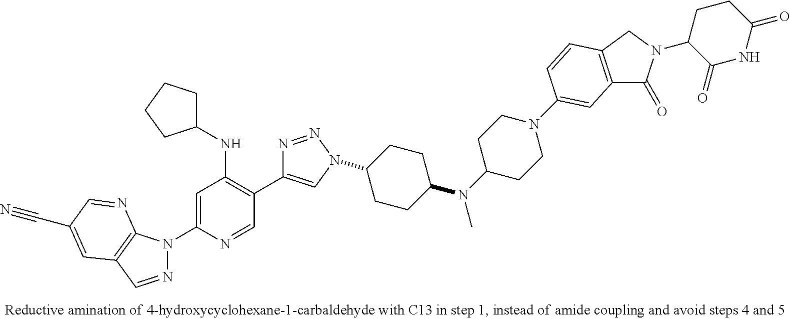 | 19% yield as a light green solid LCMS method 2: retention time: 2.371 min, 94.5% purity at 215 nm, [M + H]⁺ = 808.3; [M + 2H]²⁺ = 404.8 ¹H NMR (400 MHz, DMSO-d6) δ ppm 1.56-1.74 (m, 9H), 1.75-2.03 (m, 8H), 2.05-2.22 (m, 3H), 2.24-2.40 (m, 4H), 2.55-2.69 (m, 2H), 2.70-2.95 (m, 6H), 3.75-3.87 (m, 2H), 4.16-4.25 (m, 1H), 4.28-4.38 (m, 1H), 4.53 (br. s, 1H), 5.02-5.14 (m, 1H), 7.17 (br. s, 1H), 7.22-7.31 (m, 1H), 7.42 (d, J = 7.6 Hz, 1H), 8.91 (br. s, 1H), 10.97 (s, 1H). |
| P-45 | A9 | C16 | 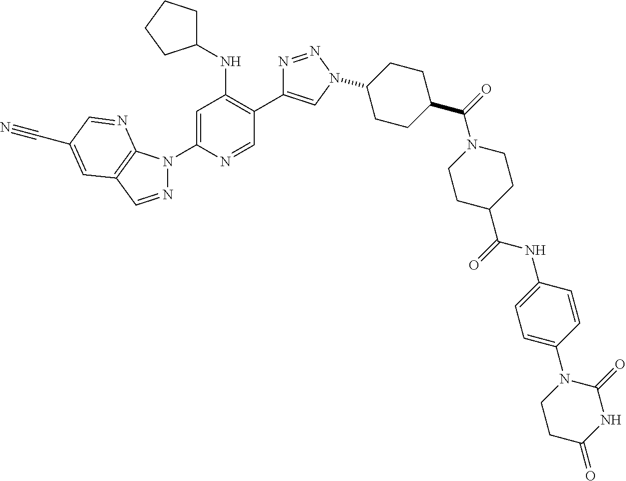 | 37% yield as a light yellow solid. LCMS method 2: retention time: 2.965 min, 97.4% purity at 215 nm, [M + H]⁺ = 796.3, [M + 2H]²⁺ = 398.7 ¹H NMR (400 MHz, DMSO-d6) δ ppm 1.40-1.50 (m, 1H), 1.59-1.75 (m, 9H), 1.83-1.98 (m, 6H), 2.07-2.28 (m, 4H), 2.61-2.77 (m, 5H), 3.05-3.18 (m, 1H), 3.70-3.76 (m, 2H), 3.82-3.97 (m, 1H), 4.01-4.14 (m, 1H), 4.40-4.51 (m, 1H), 4.54-4.66 (m, 1H), 7.24 (d, J = 7.8 Hz, 2H), 7.60 (d, J = 7.1 Hz, 2H), 7.66-9.94 (m, 7H), 9.99 (s, 1H), 10.32 (s, 1H). |

General Procedure X-5. The scheme shown below for the synthesis of P-30 is provided as a representative synthesis for General Procedure X-5.

### Example S44. 6-(5-cyano-1H-pyrazolo[3,4-b]pyridin-1-yl)-4-(cyclopentylamino)-N-((1s,4s)-4-((4-(2-(2,6-dioxopiperidin-3-yl)-1,3-dioxoisoindolin-5-yl)piperazin-1-yl)methyl)-4-hydroxycyclohexyl)nicotinamide (P-30)

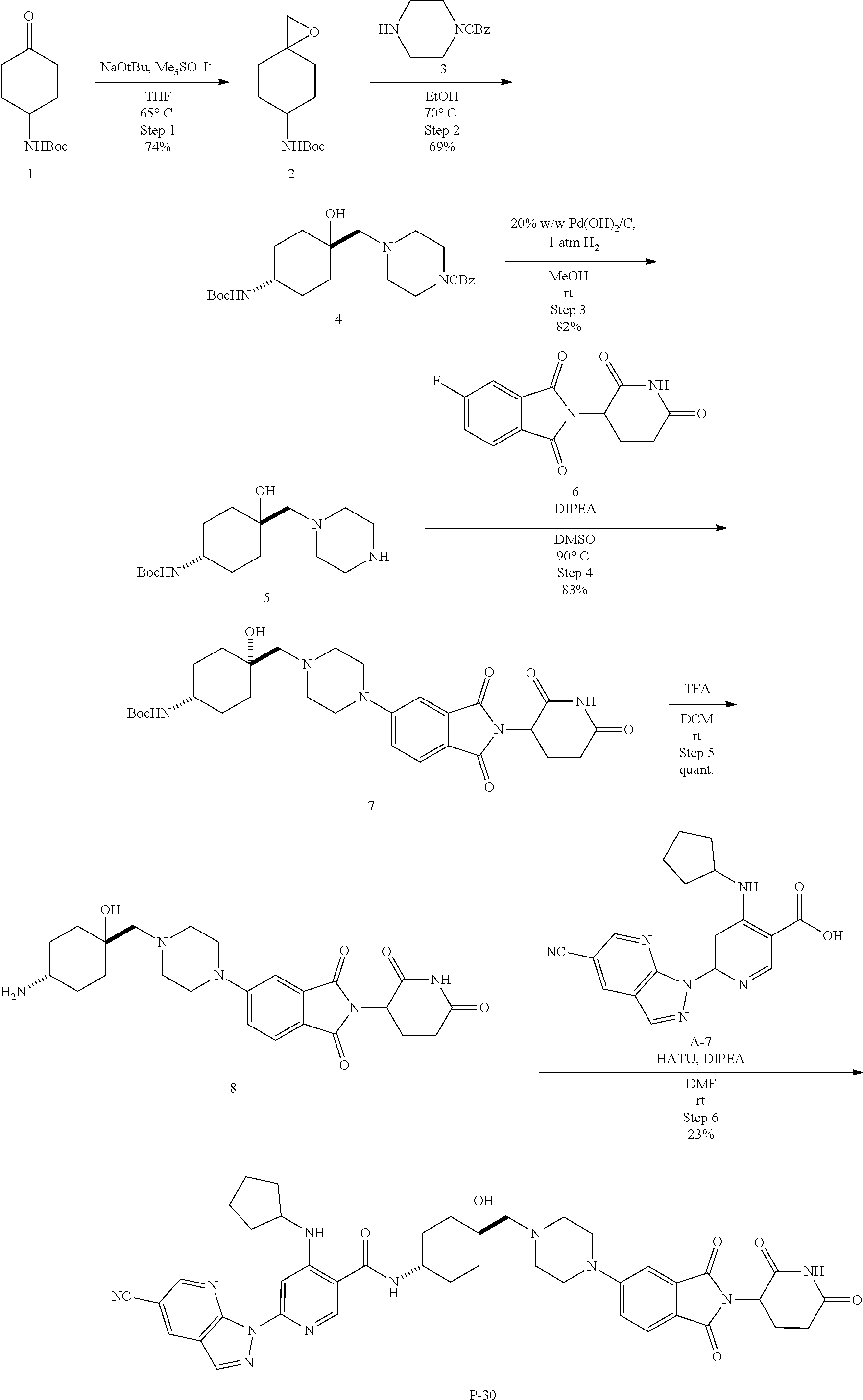

Step 1. Preparation of tert-butyl N-(1-oxaspiro[2.5]octan-6-yl)carbamate (2): To a suspension of NaOtBu (326 mg, 3.4 mmol) in dry THF (23.0 mL) was added Trimethylsulfoxonium iodide (773 mg, 3.52 mmol) in one portion and the pale yellow mixture was heated to reflux for 2 h. The mixture was cooled to room temperature and a solution of tert-butyl N-(4-oxocyclohexyl)carbamate 1 (0.5 g, 2.34 mmol) in dry THF (7.0 mL) was added dropwise over 5 min. The resulting mixture was heated to reflux for two more hours. The reaction mixture was allowed to cool down to room temperature, partitioned between water (30 mL) and EtOAc (60 mL) and the phases were separated. The aqueous phase was extracted twice with ethyl acetate, and the combined extracts were washed with brine, dried over sodium sulfate and concentrated to dryness under reduced pressure. The residue was purified by flash column chromatography (EtOAc/heptanes) to afford tert-butyl N-(1-oxaspiro[2.5]octan-6-yl)carbamate 2 (399 mg, 74% yield) as a white solid.

¹H NMR (400 MHz, CDCl₃) δ ppm 1.24-1.43 (m, 3H), 1.46 (s, 9H), 1.48-1.58 (m, 2H), 1.79-2.13 (m, 4H), 2.61-2.67 (m, 2H), 3.59 (br. s, 1H), 4.47 (br. s, 1H).

Step 2. Preparation of benzyl 4-[[4-(tert-butoxycarbonylamino)-1-hydroxy-cyclohexyl]methyl]piperazine-1-carboxylate (4): A mixture of tert-butyl N-(1-oxaspiro[2.5]octan-6-yl)carbamate 2 (395 mg, 1.74 mmol) and benzyl piperazine-1-carboxylate 3 (1.14 g, 5.21 mmol) was dissolved in dry ethanol (8.5 mL) and heated at 70° C. for 16 h. The pale-yellow mixture was allowed to cool to room temperature, partitioned between water (30 mL) and EtOAc (60 mL) and the phases were separated. The aqueous phase was extracted to EtOAc 2×. The combined extracts were washed with brine, dried over sodium sulfate and concentrated to dryness under reduced pressure. The residue was purified by flash column chromatography (EtOAc/heptanes) as a mixture of two diastereomers. Two diastereomers were separated in a ratio of 88 to 12 by chiral SFC (Phenomenex Cellulose-4 250×21.5, 5 μm 4-30% reagent alcohol as co-eluent). The main and desired product benzyl 4-[[4-(tert-butoxycarbonylamino)-1-hydroxy-cyclohexyl]methyl]piperazine-1-carbo benzyl 4-[[4-(tert-butoxycarbonylamino)-1-hydroxy-cyclohexyl]methyl]piperazine-1-carboxylate 4 (542 mg, 69% yield) was obtained as a white solid.

LCMS method 3: 99.9% purity at 215 nm, [M+H]⁺=448.2

¹H NMR (400 MHz, CDCl₃) δ ppm 0.79-0.98 (m, 1H), 1.25-1.37 (m, 3H), 1.45 (s, 9H), 1.48-1.57 (m, 2H), 1.60-1.66 (m, 2H), 1.76-1.84 (m, 2H), 2.52-2.64 (m, 4H), 2.82-2.95 (m, 1H), 3.33-3.46 (m, 1H), 3.47-3.55 (m, 4H), 4.44 (br d, J=8.1 Hz, 1H), 5.14 (s, 2H), 7.30-7.38 (m, 5H).

Step 3. Preparation of tert-butyl N-[4-hydroxy-4-(piperazin-1-ylmethyl)cyclohexyl]carbamate (5): 20% w/w Pd(OH)₂/C (95 mg) was added to a solution of benzyl 4-[[4-(tert-butoxycarbonylamino)-1-hydroxy-cyclohexyl]methyl]piperazine-1-carboxylate 4 (400 mg, 0.89 mmol) in Methanol (8.5 mL) and the resulting mixture was stirred under 1 atm H₂ using a balloon for 16 h. TLC (50% EtOAc in heptanes) shows complete conversion to product. The reaction mixture was passed through a pad of celite and the filtrate was concentrated to dryness to afford tert-butyl N-[4-hydroxy-4-(piperazin-1-ylmethyl)cyclohexyl]carbamate 5 (312 mg, 82% yield) as a white solid which was used in the next step without further purification and analysis.

Step 4. Preparation of tert-butyl N-[4-[[4-[2-(2,6-dioxo-3-piperidyl)-1,3-dioxo-isoindolin-5-yl]piperazin-1-yl]methyl]-4-hydroxy-cyclohexyl]carbamate (7): To a mixture of 2-(2,6-dioxo-3-piperidyl)-5-fluoro-isoindoline-1,3-dione 6 (162 mg, 0.59 mmol) and tert-butyl N-[4-hydroxy-4-(piperazin-1-ylmethyl)cyclohexyl]carbamate 5 (312 mg, 0.74 mmol) in anhydrous DMSO (5.0 mL) was added DIPEA (0.31 mL, 1.77 mmol) and the solution was heated at 90° C. for 16 h. The reaction mixture was directly purified by reversed phase C18 column chromatography (MeCN/0.1% aqueous formic acid) to afford tert-butyl N-[4-[[4-[2-(2,6-dioxo-3-piperidyl)-1,3-dioxo-isoindolin-5-yl]piperazin-1-yl]methyl]-4-hydroxy-cyclohexyl]carbamate 7 (280 mg, 83% yield) as a yellow solid.

LCMS method 1: 99.9% purity at 215 nm, [M+H]⁺=570.2.

¹H NMR (400 MHz, DMSO-d6) δ ppm 1.22-1.33 (m, 2H), 1.37 (s, 9H), 1.44-1.62 (m, 6H), 1.96-2.07 (m, 1H), 2.52-2.61 (m, 2H), 2.61-2.69 (m, 4H), 2.82-2.94 (m, 1H), 3.08 (s, 2H), 3.10-3.20 (m, 1H), 3.38-3.47 (m, 4H), 3.74-4.01 (m, 1H), 5.07 (dd, J=13.0, 5.4 Hz, 1H), 6.68 (d, J=7.6 Hz, 1H), 7.23 (dd, J=8.6, 1.7 Hz, 1H), 7.32 (d, J=1.5 Hz, 1H), 7.67 (d, J=8.6 Hz, 1H), 11.07 (s, 1H).

Step 5. Preparation of 5-[4-[(4-amino-1-hydroxy-cyclohexyl)methyl]piperazin-1-yl]-2-(2,6-dioxo-3-piperidyl)isoindoline-1,3-dione (8): To a solution of tert-butyl N-[4-[[4-[2-(2,6-dioxo-3-piperidyl)-1,3-dioxo-isoindolin-5-yl]piperazin-1-yl]methyl]-4-hydroxy-cyclohexyl]carbamate 7 (280 mg, 0.49 mmol) in DCM (1.0 mL) was added TFA (12 mL, 49 mmol) and the mixture was stirred at RT for 20 min. The volatiles were evaporated under reduced pressure and the residue was co-evaporated with MeCN (3×) to afford 5-[4-[(4-amino-1-hydroxy-cyclohexyl)methyl]piperazin-1-yl]-2-(2,6-dioxo-3-piperidyl)isoindoline-1,3-dione 8 (345 mg, quantitative) as a yellow solid.

LCMS method 1: 99.9% purity at 215 nm, [M+H]⁺=470.2.

Step 6. Preparation of 6-(5-cyano-1H-pyrazolo[3,4-b]pyridin-1-yl)-4-(cyclopentylamino)-N-((1s,4s)-4-((4-(2-(2,6-dioxopiperidin-3-yl)-1,3-dioxoisoindolin-5-yl)piperazin-1-yl)methyl)-4-hydroxycyclohexyl)nicotinamide (P-30): To a solution of 5-[4-[(4-amino-1-hydroxy-cyclohexyl)methyl]piperazin-1-yl]-2-(2,6-dioxo-3-piperidyl)isoindoline-1,3-dione 8 (96 mg, 0.14 mmol), HATU (52 mg, 0.14 mmol) and 6-(5-cyanopyrazolo[3,4-b]pyridin-1-yl)-4-(cyclopentylamino)pyridine-3-carboxylic acid A-7 (40 mg, 0.11 mmol) in DMF (2.0 mL) was added DIPEA (0.2 mL, 1.15 mmol) and resulting mixture was stirred for 2 h at rt. The reaction mixture was directly purified by reversed phase C18 column chromatography (MeCN/0.1% aqueous formic acid) to afford 6-(5-cyano-1H-pyrazolo[3,4-b]pyridin-1-yl)-4-(cyclopentylamino)-N-((1s,4s)-4-((4-(2-(2,6-dioxopiperidin-3-yl)-1,3-dioxoisoindolin-5-yl)piperazin-1-yl)methyl)-4-hydroxycyclohexyl)nicotinamide (P-30) (22.3 mg, 23% yield) as a yellow solid.

LCMS method 3: 98.8% purity at 215 nm, [M+H]⁺=800.2, [M+2H]²⁺=400.7

¹H NMR (400 MHz, DMSO-d6) δ ppm 1.33-1.46 (m, 2H), 1.46-1.56 (m, 2H), 1.56-1.83 (m, 10H), 1.97-2.11 (m, 3H), 2.25-2.35 (m, 2H), 2.52-2.63 (m, 2H), 2.63-2.73 (m, 4H), 2.82-2.94 (m, 1H), 3.45 (m, 4H), 3.68-3.80 (m, 1H), 3.90 (dq, J=12.0, 6.0 Hz, 1 H), 4.01 (s, 1H), 5.07 (dd, J=12.8, 5.3 Hz, 1H), 7.21-7.28 (m, 1H), 7.33 (s, 2H), 7.68 (d, J=8.6 Hz, 1H), 8.41 (d, J=7.6 Hz, 1H), 8.60-8.66 (m, 2H), 8.69 (d, J=6.1 Hz, 1H), 9.01 (d, J=2.0 Hz, 1H), 9.05 (d, J=2.0 Hz, 1H), 11.07 (s, 1H).

Table 34 summarizes the compounds prepared using General Procedure X-5.

TABLE 35
Final Compounds Prepared via General Procedure X-5

| Compound | TMB | CBM | | |
|---|---|---|---|---|
| No. | Portion | Portion | Structure | Characterization |
| P-30 | A7 | | 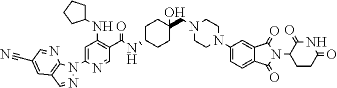 | 24% yield as a yellow solid LCMS method 2: retention time: 2.371 min, 98.8% purity at 215 nm, [M + H]⁺ = 800.2, [M + 2H]²⁺ = 400.7 H NMR (400 MHz, DMSO-d6) δ ppm 1.33-1.46 (m, 2H), 1.46-1.56 (m, 2H), 1.56-1.83 (m, 10H), 1.97-2.11 (m, 3H), 2.25-2.35 (m, 2H),2.52-2.63 (m, 2H), 2.63-2.73 (m, 4H), 2.82-2.94 (m, 1H), 3.45 (m, 4H), 3.68-3.80 (m, 1H), 3.90 (dq, J = 12.0, 6.0 Hz, 1H), 4.01 (s, 1H),5.07 (dd, J = 12.8, 5.3 Hz, 1H), 7.21-7.28 (m, 1H), 7.33 (s, 2H), 7.68 (d, J = 8.6 Hz, 1H), 8.41 (d, J = 7.6 Hz, 1H), 8.60-8.66 (m, 2H), 8.69 (d, J = 6.1 Hz, 1H), 9.01 (d, J = 2.0 Hz, 1H), 9.05 (d, J = 2.0 Hz, 1H), 11.07 (s, 1H). |

General Procedure X-6. The scheme shown below for the synthesis of P-55 is provided as a representative synthesis for General Procedure X-6.

### Example S45. 6-(5-cyano-1H-pyrazolo[3,4-b]pyridin-1-yl)-4-(cyclopentylamino)-N-((1s,4s)-4-((4-(2-(2,6-dioxopiperidin-3-yl)-1,3-dioxoisoindolin-5-yl)piperazin-1-yl)methyl)-4-hydroxycyclohexyl)nicotinamide (P-55)

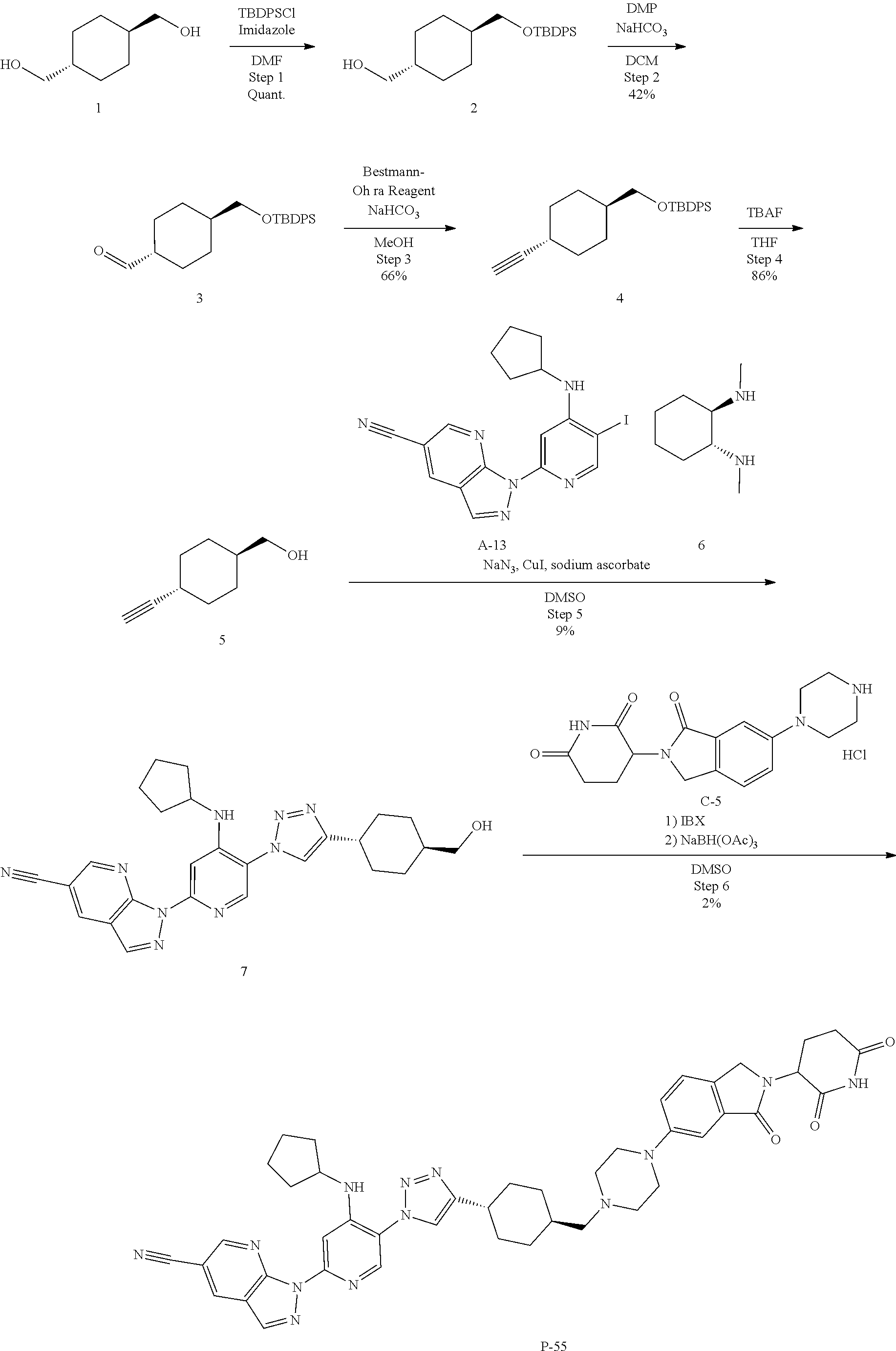

Step 1. Preparation of [4-[[tert-Butyl(diphenyl)silyl]oxymethyl]cyclohexyl]methanol (2): 1 (1.05 g, 7.29 mmol) was dissolved in DMF (4.5 mL, 0.8 M) at room temperature, then TBDPSCl (0.95 mL, 3.64 mmol, 0.5 eq.) and imidazole (0.3 g, 4.37 mmol, 0.6 eq.) were added. After 1 h, TLC (25% EtOAc in heptanes, using KMnO₄ stain) shows good conversion. The reaction was let overnight at rt. After stirring at room temperature for 16 hours, the reaction was quenched by 40 mL brine, partitioned with 50 mL EtOAc. The aqueous phase was extracted with EtOAc (1×). Combined organic layers were washed with brine (4×) and dried over Na₂SO₄. Filtration and concentration afforded 2 (2.018 g, 99% yield) as a light yellow oil.

LCMS method 1: retention time: 2.320 min, 86.7% purity at 215 nm, [M+H]⁺=383.2.

¹H NMR (400 MHz, CDCl₃) δ ppm 0.94-1.03 (m, 4H), 1.06 (s, 9H), 1.36 (br s, 1H), 1.48 (dtd, J=11.7, 6.1, 3.5 Hz, 2H), 1.79-1.89 (m, 4H), 3.48 (dt, J=6.4, 3.3 Hz, 4H), 7.35-7.46 (m, 6H), 7.67 (dd, J=7.7, 1.6 Hz, 4H).

Step 2. Preparation of 4-[[tert-Butyl(diphenyl)silyl]oxymethyl]cyclohexanecarbaldehyde (3): To a solution of 2 (1.39 g, 3.63 mmol, 1.0 eq.) and NaHCO₃ (1.1 g, 10.9 mmol, 3.0 eq.) in DCM (36 mL, 0.1 M) at 0° C. was added Dess-Martin periodinane (2.47 g, 5.81 mmol, 1.6 eq.). The resulting mixture was stirred at 0° C. for 1 hour. The reaction mixture was filtered on Celite, rinsing with DCM. The filtrate was evaporated under reduced pressure, dry-packing on silica. The crude mixture was purified by normal phase flash chromatography (EtOAc/heptanes) to afford 3 (594 mg, 42% yield) as a colorless oil.

¹H NMR (400 MHz, CDCl₃) δ ppm 1.00-1.12 (m, 11H), 1.22-1.35 (m, 2H), 1.46-1.56 (m, 1H), 1.93 (br dd, J=13.2, 2.9 Hz, 2H), 1.98-2.05 (m, 2H), 2.14-2.23 (m, 1H), 3.50 (d, J=6.1 Hz, 2H), 7.36-7.47 (m, 6H), 7.64-7.69 (m, 4H), 9.63 (d, J=1.2 Hz, 1H).

Step 3. Preparation of tert-Butyl-1(4-ethynylcyclohexyl)methoxyl-diphenyl-silane (4): 3 (375 mg, 0.990 mmol, 1.0 eq.) and K2CO3 (254 mg, 1.97 mmol, 2.0 eq.) were charged in a flame-dried round bottom flask. Vacuum was applied and the flask was back-filled with nitrogen (repeated three times). Methanol (6.56 mL, 0.5 M) was added and the mixture was stirred at room temperature under a nitrogen atmosphere for 20 minutes. Bestmann-Ohira reagent (2.27 mL, 1.18 mmol, 1.2 eq.) as a 10% solution in MeCN was added to the reaction mixture at room temperature. The resulting mixture was stirred at room temperature under a nitrogen atmosphere for 4 hours. The reaction mixture was diluted with water and EtOAc. The aqueous phase was extracted EtOAc (1×). Organic layers were washed with saturated NaHCO₃aqueous solution and brine, dried over Na₂SO₄ and concentrated to dryness. The crude mixture was purified by normal phase flash chromatography (EtOAc/heptanes) to afford 4 (246 mg, 66% yield) as a colorless oil.

LCMS method 3: retention time: 2.688 min, 99.9% purity at 215 nm, [M+H]⁺=377.2.

¹H NMR (400 MHz, CDCl₃) δ ppm 0.93-1.04 (m, 2H), 1.06 (s, 9H), 1.39 (qd, J=12.8, 3.3 Hz, 2H), 1.49-1.55 (m, 1H), 1.78-1.87 (m, 2H), 1.99-2.06 (m, 3H), 2.14-2.23 (m, 1H), 3.46 (d, J=6.4 Hz, 2H), 7.35-7.47 (m, 6H), 7.63-7.70 (m, 4H).

Step 4. Preparation of (4-Ethynylcyclohexyl)methanol (5): 4 (65.6 mg, 0.170 mmol, 1.0 eq.) in THF (2.52 mL, 0.69 M) at 0° C. was added a 1 M solution of TBAF in THF (0.7 mL, 0.700 mmol, 4.0 eq.) at 0° C. The reaction was stirred at room temperature for 16 h. Volatiles were evaporated and the crude was purified by reverse phase flash chromatography (0.1% F.A. aq./MeCN) to afford 5 (20.9 mg, 86% yield).

¹H NMR (400 MHz, CDCl₃) δ ppm 0.91-1.03 (m, 2H), 1.34-1.55 (m, 4H), 1.79-1.87 (m, 2H), 2.00-2.08 (m, 3H), 2.16-2.25 (m, 1H), 3.45 (d, J=6.4 Hz, 2H).

Step 5. Preparation of 1-[4-(Cyclopentylamino)-5-[4-[4-(hydroxymethyl)cyclohexyl]triazol-1-yl]-2-pyridyl]pyrazolo[3,4-b]pyridine-5-carbonitrileacetate (7): A solution of 5 (20.9 mg, 0.150 mmol, 1.0 eq.), A-13 (65.0 mg, 0.150 mmol, 1.0 eq.), CuI (10.0 mg, 0.050 mmol, 0.53 eq.), sodium ascorbate (12.0 mg, 0.060 mmol, eq.), 6 (11.9 μL, 0.080 mmol, 0.5 eq.) and NaN₃ (19.6 mg, 0.300 mmol, 2.0 eq.) in DMSO (1.0 mL, 0.15 M) was stirred at room temperature for 16 h. After 16 h, the reaction mixture was purified by reverse phase flash chromatography (MeCN/0.1% F.A. aq.) to afford 7 (10.5 mg, 9% yield) as a green solid.

LCMS method 1: retention time: 1.704 min, 67.8% purity at 215 nm, [M+H]⁺=484.4.

¹H NMR (400 MHz, DMSO-d6) δ ppm 1.02-1.14 (m, 2H), 1.34-1.70 (m, 9H), 1.84-1.90 (m, 2H), 2.02-2.09 (m, 2H), 2.10-2.18 (m, 2H), 2.66-2.75 (m, 1H), 3.27 (t, J=Hz, 2H), 3.94 (br dd, J=12.8, 7.0 Hz, 1H), 4.42 (t, J=5.4 Hz, 1H), 6.83 (br d, J=6.6 Hz, 1H), 7.54 (br s, 1H), 8.35-8.41 (m, 1H), 8.44 (s, 1H), 8.67 (s, 1H), 9.03 (d, J=2.0 Hz, 1H), 9.08 (s, 1H).

Step 6. Preparation of 1-[4-(Cyclopentylamino)-5-[4-[4-[[4-[2-(2,6-dioxo-3-piperidyl)-3-oxo-isoindolin-5-yl]piperazin-1-yl]methyl]cyclohexyl]triazol-1-yl]-2-pyridyl]pyrazolo[3,4-b]pyridine-5-carbonitrile (P-55): To a solution of 7 (100 mg, 0.210 mmol, 1.0 eq.) in DMSO (0.20 mL, 1.0 M) at room temperature was added IBX (75.2 mg, 0.270 mmol, 1.3 eq.) in one portion and the solution was stirred at room temperature for 16 h. The solution was added dropwise over 3 min to a solution of C-5 (75.4 mg, 0.210 mmol, 1.0 eq.), and NaBH(OAc)₃ (57.0 mg, 0.270 mmol, 1.3 eq.) in DMSO (0.20 mL, 0.5 M). The resulting mixture was stirred at room temperature for 16 h. Reaction mixture was purified by reverse phase flash chromatography (MeCN/0.1% F.A. aq.) to afford impure material. The material was purified by preparative HPLC to afford P-55 (4.0 mg, 2% yield) as a white solid.

LCMS method 2: retention time: 2.495 min, 99.5% purity at 215 nm, [M+H]⁺=794.5, [M+2H]²⁺=397.8.

¹H NMR (400 MHz, DMSO-d6) δ ppm 0.78-0.95 (m, 1H), 1.00-1.16 (m, 2H), 1.20-1.40 (m, 2H), 1.45-1.71 (m, 9H), 1.89-2.25 (m, 9H), 2.34-2.42 (m, 1H), 2.55-2.62 (m, 1H), 2.67-2.81 (m, 1H), 2.83-2.99 (m, 1H), 3.10-3.25 (m, 5H), 3.86-4.02 (m, 1H), 4.10-4.41 (m, 2H), 5.02-5.18 (m, 1H), 6.76-6.92 (m, 1H), 7.08-7.34 (m, 2H), 7.35-7.61 (m, 2H), 8.29-8.53 (m, 2H), 8.67 (s, 1H), 8.9-9.2 (d, J=16.6 Hz, 2H), 10.96 (s, 1H).

Table 35 summarizes the compounds prepared using General Procedure X-6.

TABLE 35
Final Compounds Prepared via General Procedure X-6

| Compound | TMB | CBM | | |
|---|---|---|---|---|
| No. | Portion | Portion | Structure | Characterization |
| P-33 | A13 | C9 | 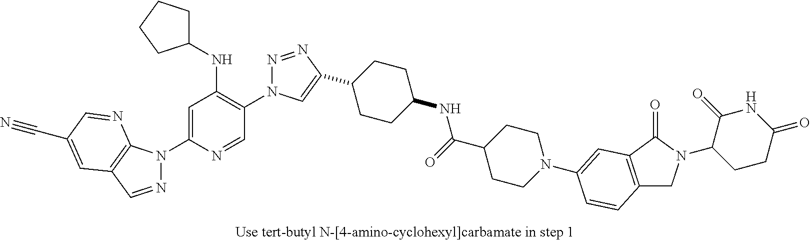 | 24 % yield as a white solid. LCMS method 2: retention time: 3.316 min,98.2 % purity at 215 nm, [M + H]⁺ = 822.2; [M + 2H]²⁺ = 411.7 ¹H NMR (400 MHz, DMSO-d6) δ ppm 1.27-1.40 (m, 3H), 1.50-1.60 (m, 6H), 1.66-1.78 (m, 6H), 1.87-1.94 (m, 2H), 2.01-2.08 (m, 2H), 2.11-2.19 (m, 2H), 2.29-2.40 (m, 2H), 2.55-2.63 (m, 1H), 2.68-2.77 (m, 3H), 2.85-2.96 (m, 1H), 3.55-3.66 (m, 1H), 3.76-3.83 (m, 2H), 3.89-3.99 (m, 1H), 4.17-4.24 (m, 1H), 4.30-4.37 (m, 1H), 5.10 (dd, J = 13.2, 5.1 Hz, 1H), 6.82 (d, J = 6.6 Hz, 1H), 7.18 (s, 1H), 7.25-7.30 (m, 1H), 7.43 (d, J = 8.6 Hz, 1H), 7.54 (s, 1H), 7.77 (d, J = 7.8 Hz, 1H), 8.38 (s, 1H), 8.46 (s, 1H), 8.67 (s, 1H), 9.04 (d, J = 2.0 Hz, 1H), 9.08 (d, J = 2.0 Hz, 1H), 10.97 (s, 1H). |
| P-55 | A13 | C5 | 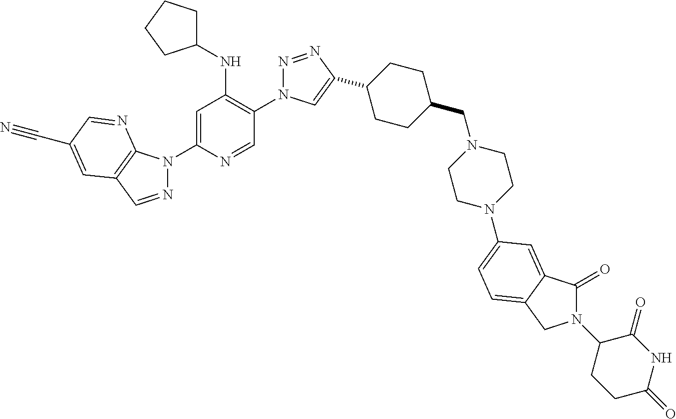 | 2.4 % yield as a white solid. LCMS method 2: retention time: 2.495 min, 99.5 % purity at 215 nm, [M + H]⁺ = 794.5, [M + 2H]²⁺ = 397.8 ¹H NMR (400 MHz, DMSO-d6) δ ppm 0.78-0.95 (m, 1H), 1.00-1.16 (m, 2H), 1.20-1.40 (m, 2H), 1.45-1.71 (m, 9H), 1.89-2.25 (m, 9H), 2.34-2.42 (m, 1H), 2.55-2.62 (m, 1H), 2.67-2.81 (m, 1H), 2.83-2.99 (m, 1H), 3.10-3.25 (m, 5H), 3.86-4.02 (m, 1H), 4.10-4.41 (m, 2H), 5.02-5.18 (m, 1H), 6.76-6.92 (m, 1H), 7.08-7.34 (m, 2H), 7.35-7.61 (m, 2H), 8.29-8.53 (m, 2H), 8.67 (s, 1H), 8.9-9.2 (d, J = 16.6 Hz, 2H), 10.96 (s, 1H). Two protons were partially under the peak of DMSO by ¹H NMR. Five protons were partially under the peak of water by ¹H NMR. |

### General Procedure X-7

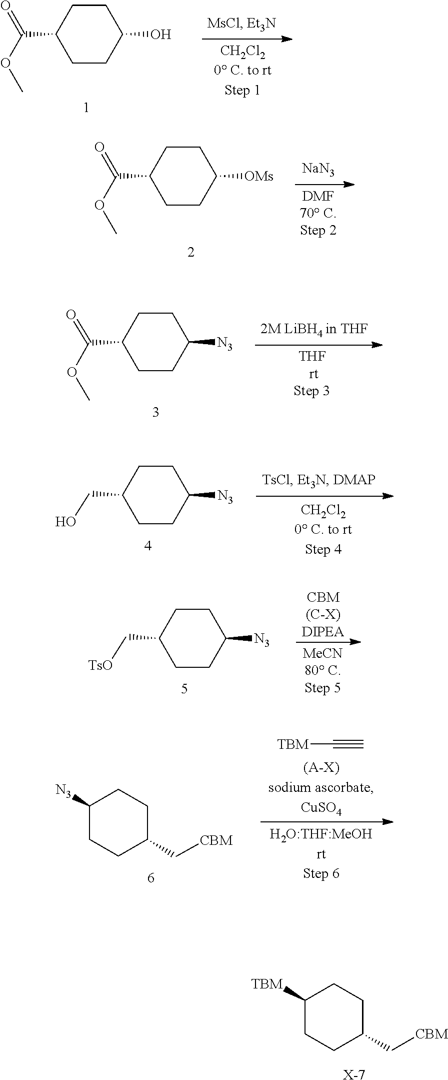

Step 1. Preparation of tert-Butyl N-[4-(2-hydroxyethyl)cyclohexyl]carbamate (2): To a solution of tert-butyl N-(4-hydroxycyclohexyl)carbamate 1 (1 eq.) and triethylamine (1.2 eq) in CH₂Cl₂ at 0° C. was added dropwise methanesulfonyl chloride (1.1 eq.). After 2h, TLC showed totat conversion of 1. The mixture was partitioned between EtOAc and water. The organic phase was washed once with water, once with 1 N HCl, dried over magnesium sulfate, filtered and concentrated under reduced pressure to give 2.

Step 2. Preparation of Methyl 4-azidocyclohexanecarboxylate (3): To a solution of methyl 4-methylsulfonyloxycyclohexanecarboxylate 2 (leg.) in DMF (0.5M) and NaN₃ (2 eq.). Then the mixture was stirred at 70° C. After TLC showed total consumption of 2, the mixture was partitioned between EtOAc and water. The organic phase was washed once with water, once with sat. NaHCO₃, then dried over magnesium sulfate, filtered and concentrated under reduced pressure. The residue was then purified, tractions were combined and concentrated to give 3.

Step 3. Preparation of (4-Azidocyclohexyl)methanol (4): To a solution of methyl 4-azidocyclohexanecarboxylate 3 (1 eq.) in THF (0.33 M) was added 2 M LiBH₄ in THF (3 eq.) and the mixture was stirred at room temperature. After TLC showed completion of the reaction, the mixture was quenched with sat. aq. NaHCO₃ and extracted with EtOAc (3×). Combined organic phases were dried over MgSO₄, filtered and concentrated. The crude was purified, fractions were concentrated to give 4.

Step 4. Preparation of (4-Azidocyclohexyl)methyl 4-methylbenzenesulfonate (5): To a solution of (4-azidocyclohexyl)methanol 4 (1 eq.), DMAP (0.3 eq.) and Et3N (eq.) and in CH₂Cl₂ (0.7 M) at 0° C. was added TsCl (1.2 eq.), which was then allowed to warm-up to room temperature. After 16h, the reaction was stopped and the mixture was partitioned between EtOAc and water. The organic phase was washed once with water and once with 1 N HCl, dried over magnesium sulfate, filtered and evaporated under reduced pressure. The crude was purified, fractions were combined and concentrated to give 5.

Step 5. Preparation of (6): To a solution of CBM C-X (1 eq.) in MeCN (5.4 mL) was added DIPEA (2 eq.) and (4-azidocyclohexyl)methyl 4-methylbenzenesulfonate 5 (1.5 eq.) at room temperature. Then, the solution was stirred at 80° C. After 16 h, the reaction was cooled down to room temperature. The reaction mixture was purified and fractions were combined and concentrated to give 6.

Step 6. Preparation of final product X-7: Azide 6 (1 eq.), TBM A-X (1 eq.), sodium ascorbate (2 eq.) and CuSO₄ (0.2 eq.) were charged in a small reaction vial and solubilized in water:THF:methanol (1:1:1, 0.1 M) and stirred at room temperature. After LCMS showed full conversion. The suspension was filtered, the filtrate was concentrated and the residue was purified. Fractions were combined, concentrated to give X-7.

### Example S46. 2-(4-(((1r,4r)-4-(4-(6-(5-Cyano-1H-pyrazolo[3,4-b]pyridin-1-yl)-4-(cyclopentylamino)pyridin-3-yl)-1H-1,2,3-triazol-1-yl)cyclohexyl)methyl)piperazin-1-yl)-N-(4-(2,6-dioxopiperidin-3-yl)phenyl)acetamide (P-40)

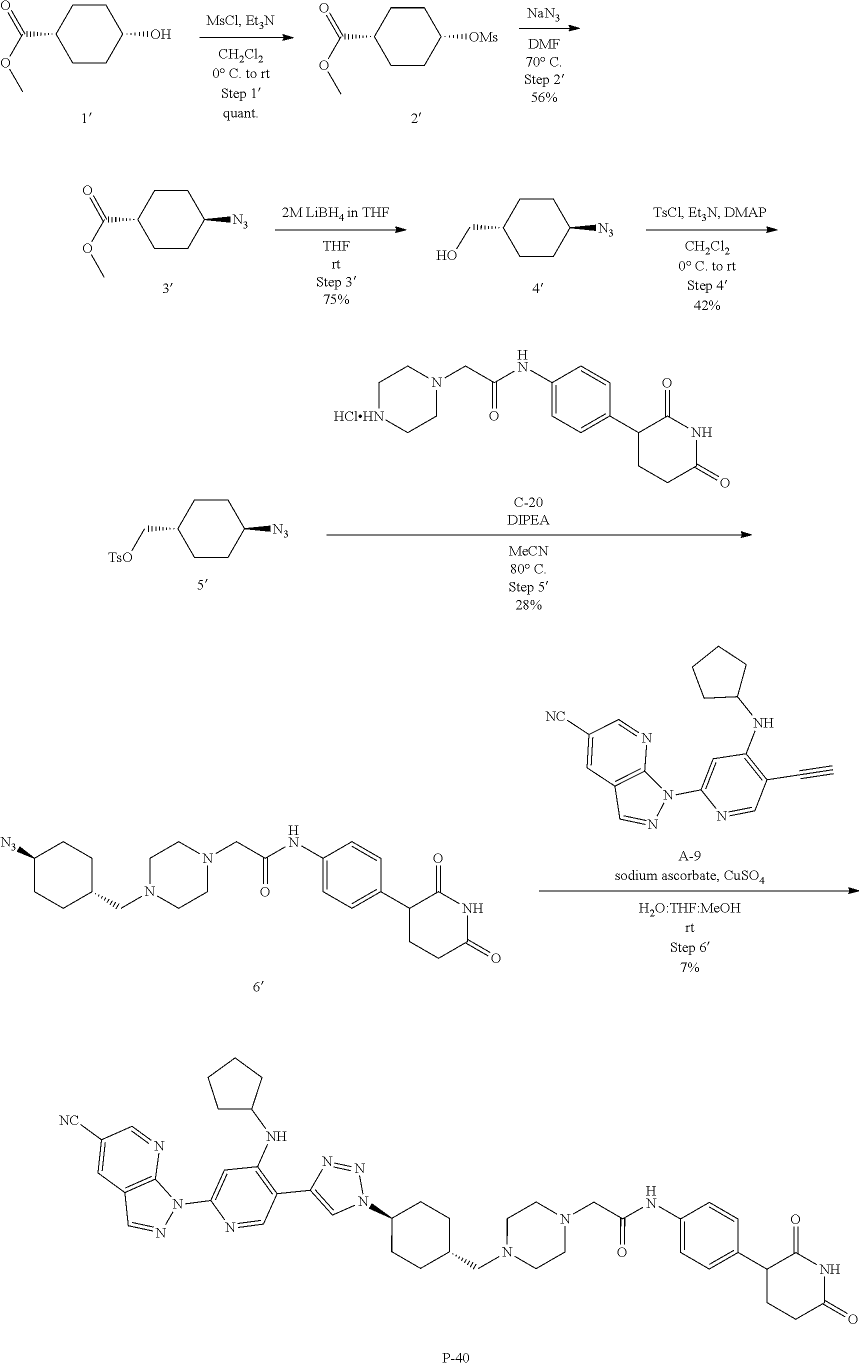

Step 1′. Preparation of Methyl 4-methylsulfonyloxycyclohexanecarboxylate (2′): To a solution of tert-butyl N-(4-hydroxycyclohexyl)carbamate 1′ (1.06 mL, 4.64 mmol, 1 eq.) and triethylamine (776.91 uL, 5.57 mmol, 1.2 eq) in CH₂Cl₂ at 0° C. was added dropwise methanesulfonyl chloride (395.51 uL, 5.11 mmol, 1.1 eq.). After 2h, TLC (Heptane/EtOAc; 4/6, revelator: KMnO₄) showed total conversion of V. The mixture was partitioned between EtOAc and water. The organic phase was washed once with water, once with 1 N HCl, dried over magnesium sulfate, filtered and concentrated under reduced pressure to give 2′ (1.56 g, quant.) as a colorless oil, which was used as is into the next step.

¹H NMR (400 MHz, chloroform-d) δ ppm 1.67-1.83 (m, 4H), 1.86-1.98 (m, 2H), 1.99-2.08 (m, 2H), 2.34-2.45 (m, 1H), 3.01 (s, 3H), 3.68 (s, 3H), 4.87-4.94 (m, 1H).

Step 2′. Preparation of Methyl 4-azidocyclohexanecarboxylate (3′): To a solution of methyl 4-methylsulfonyloxycyclohexanecarboxylate 2′ (1.57 g, 6.64 mmol, 1 eq.) in DMF (10 mL) and NaN₃ (863.35 mg, 13.28 mmol, 2 eq.). Then the mixture was stirred at 70° C. After 16h, TLC (heptane/EtOAc 5/5) showed total consumption of 2. The mixture was partitioned between EtOAc and water. The organic phase was washed once with water, once with sat. NaHCO₃, then dried over magnesium sulfate, filtered and concentrated under reduced pressure. The residue was then purified by normal phase flash chromatography (40 g silica column, solid deposit, elution: 0 to 100% EtOAc/Heptane over 20 CV. Fractions were combined and concentrated to give 3′ (681 mg, 56% yield) as a colorless oil.

¹H NMR (400 MHz, chloroform-d) δ ppm 1.30-1.42 (m, 2H), 1.45-1.59 (m, 2H), 2.01-2.10 (m, 4H), 2.28 (tt, J=11.6, 3.1 Hz, 1H), 3.30 (br s, 1H), 3.67 (s, 3H).

Step 3′. Preparation of (4-Azidocyclohexyl)methanol (4′): To a solution of methyl 4-azidocyclohexanecarboxylate 3′ (2.67 g, 14.57 mmol, 1 eq.) in THF (42 mL) was added 2 M LiBH₄ in THF (21.86 mL, 43.72 mmol, 3 eq.) and the mixture was stirred at room temperature. After 16h, TLC (Hexane/EtOAc 5/5) showed completion of the reaction. The mixture was quenched with sat. aq. NaHCO₃ and extracted with EtOAc (3×). Combined organic phases were dried over MgSO₄, filtered and concentrated. The crude was purified by normal phase chromatography (40 g silica column, solid deposit, elution: 0 to 70% EtOAc/heptane over 20 CV). Fractions were concentrated to give 4′ (1.71 g, 75% yield) as a colorless oil.

¹H NMR (400 MHz, chloroform-d) δ ppm 0.96-1.09 (m, 2H), 1.27-1.39 (m, 2H), 1.40-1.52 (m, 1H), 1.79-1.92 (m, 3H), 1.99-2.07 (m, 2H), 3.15-3.28 (m, 1H), 3.44 (d, J=6.4 Hz, 2H).

Step 4′. Preparation of (4-Azidocyclohexyl)methyl 4-methylbenzenesulfonate (5′): To a solution of (4-azidocyclohexyl)methanol 4′ (0.75 g, 4.81 mmol, 1 eq.), DMAP (176.41 mg, 1.44 mmol, 0.3 eq.) and Et3N (1.34 mL, 9.63 mmol, 2 eq.) and in CH₂Cl₂ (7 mL) at C. was added TsCl (0.45 mL, 5.78 mmol, 1.2 eq.), which was then allowed to warm-up to room temperature. After 16 h, TLC (Heptane/EtOAc; 4/6, revelator: KMnO₄) showed conversion but it was not complete. The reaction was stopped and the mixture was partitioned between EtOAc and water. The organic phase was washed once with water and once with 1 N HCl, dried over magnesium sulfate, filtered and evaporated under reduced pressure. The crude was purified by normal phase flash chromatography (40 g column, solid deposit, 0 to 100% EtOAc/heptane over 20 CV) Fractions were combined and concentrated to give 5′ (660 mg, 42% yield) as a white solid.

¹H NMR (400 MHz, chloroform-d) δ ppm 0.97-1.12 (m, 2H), 1.24-1.39 (m, 2H), 1.60-1.71 (m, 1H), 1.77-1.87 (m, 2H), 1.98-2.07 (m, 2H), 2.46 (s, 3H), 3.20 (tt, J=11.5, 4.2 Hz, 1H), 3.84 (d, J=6.4 Hz, 2H), 7.36 (d, J=7.8 Hz, 2H), 7.79 (d, J=8.3 Hz, 2H).

Step 5′. Preparation of 2-[1-[(4-Azidocyclohexyl)methyl]-4-piperidyl]-N-[4-(2,6-dioxo-3-piperidyl)phenyl]acetamide (6′): To a solution of CBM N-[4-(2,6-dioxo-3-piperidyl)phenyl]-2-(4-piperidyl)acetamide hydrochloride C-20 (200 mg, 0.55 mmol, 1 eq.) in MeCN (5.4 mL) was added DIPEA (0.19 mL, 1.09 mmol, 2 eq.) and (4-azidocyclohexyl)methyl 4-methylbenzenesulfonate 5′ (253.69 mg, 0.82 mmol, 1.5 eq.) at room temperature. Then, the solution was stirred at 80° C. After 16 h, LCMS showed almost full conversion. The solution was cooled down to room temperature and the solution in MeCN was purified by reverse phase flash chromatography (100 g C18 column, liquid deposit (MeCN), elution: 5% MeCN/0.1% HCOOH over 5 CV, then 5 to 100% MeCN/0.1% HCOOH over 10 CV, then 100% MeCN/0.1% HCOOH over 5 CV). Fractions were combined and concentrated to give 6′ (70.4 mg, 28% yield) as a brown solid.

LCMS method 1: 99.9% purity at 215 nm, [M+H]⁺=468.2.

Step 6′. Preparation of 2-(4-O(1r,4r)-4-(4-(6-(5-Cyano-1H-pyrazolo[3,4-b]pyridin-1-yl)-4-(cyclopentylamino)pyridin-3-yl)-1H-1,2,3-triazol-1-yl)cyclohexyl)methyl)piperazin-1-yl)-N-(4-(2,6-dioxopiperidin-3-yl)phenyl)acetamide (P-40): 2-[4-[(4-Azidocyclohexyl)methyl]piperazin-1-yl]-N-[4-(2,6-dioxo-3-piperidyl)phenyl]acetamide 6′ (70.4 mg, 0.15 mmol, 1 eq.), TBM 1-[4-(1-ethylbutylamino)-5-ethynyl-2-pyridyl]pyrazolo[3,4-b]pyridine-5-carbonitrile A-9 (51.86 mg, 0.15 mmol, 1 eq.), sodium ascorbate (59.63 mg, 0.30 mmol, 2 eq.) and CuSO₄ (4.81 mg, 0.03 mmol, 0.2 eq.) were charged in a small reaction vial and solubilized in water:THF:methanol (1:1:1, 1.5 mL) and stirred at room temperature. After 16h, LCMS showed full conversion. The suspension was filtered, the filtrate was concentrated and the residue purified by reverse phase flash chromatography (150 g C18 column, liquid deposit, elution: 5% MeCN/0.1% HCOOH over 5 CV, then 5 to 100% MeCN/0.1% HCOOH over 20 CV, then 100% MeCN/0.1% HCOOH over 5 CV). Fractions were combined, concentrated and lyophilised to give P-40 (8.17 mg, 7% yield) as a white solid.

LCMS method 2: 97.3% purity at 215 nm, [M+H]⁺=796.3; [M+2H]²⁺=398.8

¹H NMR (400 MHz, DMSO-d6) ppm 1.00-1.35 (m, 4H), 1.44-2.31 (m, 21H), 2.60-2.64 (m, 1H), 3.06-3.17 (m, 2H), 3.44-3.54 (m, 3H), 3.75-3.86 (m, 2H), 3.86-4.03 (m, 2H), 4.50-4.61 (m, 1H), 7.16 (d, J=8.1 Hz, 2H), 7.57 (d, J=7.8 Hz, 2H), 8.52-9.36 (m, 7H), 9.68 (s, 1H), 10.80 (s, 1H).

Table 36 summarizes the compounds prepared using General Procedure X-7.

TABLE 36
Final Compounds Prepared via General Procedure X-7

| Compound | TMB | CBM | | |
|---|---|---|---|---|
| No. | Portion | Portion | Structure | Characterization |
| P-34 | A9 | C16 | 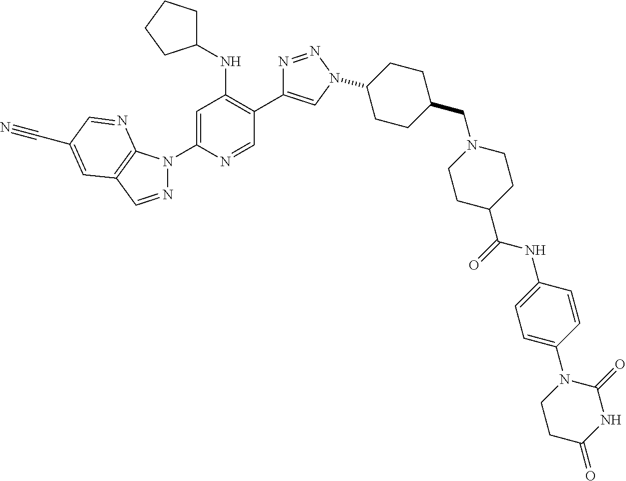 | 12% yield as a white solid. LCMS method 2: retention time: 2.332 min, 97.4% purity at 215 nm, [M + H]⁺ = 782.3, [M + 2H]²⁺ = 391.8 ¹H NMR (400 MHz, DMSO-d6) δ ppm 1.08-1.24 (m, 2H), 1.51-1.80 (m, 11H), 1.80-2.01 (m, 6H), 2.06-2.26 (m, 6H), 2.26-2.36 (m, 1H), 2.69 (t, J = 6.6 Hz, 2H), 2.88-2.96 (m, 2H), 3.73 (t, J = 6.6 Hz, 2H), 3.99-4.04 (m, J = 10.6, 5.7 Hz, 1H), 4.50-4.62 (m, 1H), 7.23 (d, J = 8.8 Hz, 2H), 7.27-7.48 (m, 1H), 7.60 (d, J = 8.6 Hz, 2H), 8.51 (d, J = 5.9 Hz, 1H), 8.63 (br. s, 2H), 8.89 (s, 1H), 8.98-9.13 (m, 2H), 9.92 (s, 1H), 10.32 (br. s, 1H). |
| P-40 | A9 | C20 | 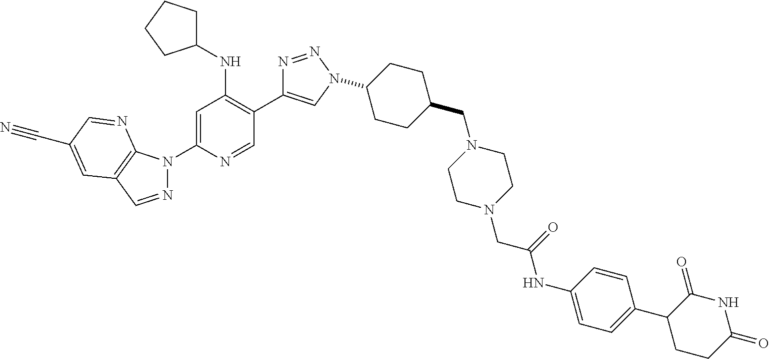 | 6.6% yield as a white solid. LCMS method 2: retention time: 2.358 min, 97.3% purity at 215 nm, [M + H]⁺ = 796.3, [M + 2H]²⁺ = 398.8 ¹H NMR (400 MHz, DMSO-d6) δ ppm 1.00-1.35 (m, 4H), 1.44-2.31 (m, 21H), 2.60-2.64 (m, 1H), 3.06-3.17 (m, 2H), 3.44-3.54 (m, 3H), 3.75-3.86 (m, 2H), 3.86-4.03 (m, 2H), 4.50-4.61 (m, 1H), 7.16 (d, J = 8.1 Hz, 2H), 7.57 (d, J = 7.8 Hz, 2H), 8.52-9.36 (m, 7H), 9.68 (s, 1H), 10.80 (s, 1H). Three protons were partially under the peak of water in the ¹H NMR. |
| P-44 | A9 | C122 | 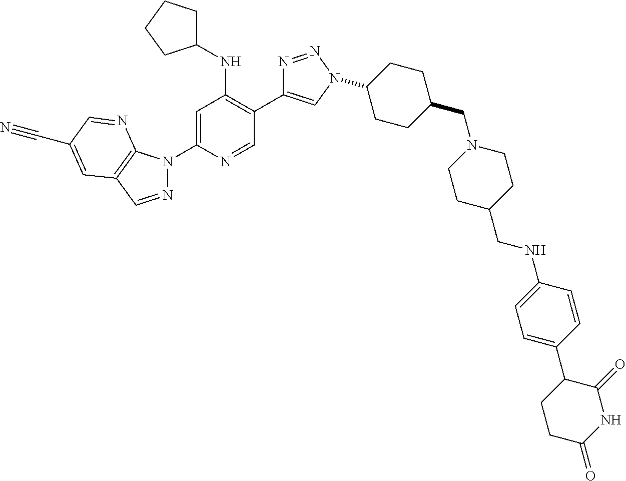 | 6.6% yield as a light yellow solid. LCMS method 2: retention time: 2.358 min, 97.3% purity at 215 nm, [M + H]⁺ = 767.3, [M + 2H]²⁺ = 384.3, [M + H]³⁺ = 256.6 ¹H NMR (400 MHz, DMSO-d6) δ ppm 1.05-1.30 (m, 4H), 1.48-1.78 (m, 10H), 1.82-2.00 (m, 6H), 2.04-2.22 (m, 6H), 2.37-2.44 (m, 1H), 2.56-2.65 (m, 1H), 2.81-2.92 (m, 4H), 3.62 (dd, J = 9.9, 5.0 Hz, 2H), 3.77-3.91 (m, 1H), 4.45-4.65 (m, 1H), 5.50-5.65 (m, 1H), 6.51 (d, J = 8.1 Hz, 2H), 6.88 (d, J = 8.3 Hz, 2H), 7.67-10.06 (m, 8H), 10.72 (s, 1H). |
| P-47 | A9 | C16 | 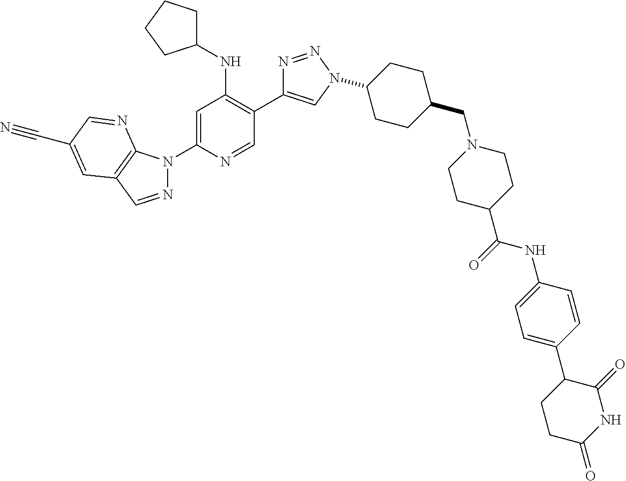 | 9.1% yield as a light yellow solid. LCMS method 2: retention time: 2.381 min, 98.4 % purity at 215 nm, [M + H]⁺ = 781.3, [M + 2H]²⁺ = 391.2 ¹H NMR (400 MHz, DMSO-d6) δ ppm 1.18-1.30 (m, 2H), 1.58-1.73 (m, 5H), 1.74-1.81 (m, 2H), 1.88-1.95 (m, 4H), 1.97-2.04 (m, 2H), 2.05-2.19 (m, 5H), 2.21-2.30 (m, 3H), 2.60-2.70 (m, 3H), 3.21-3.33 (m, 6H), 3.76-3.85 (m, 1H), 3.86-3.98 (m, 1H), 4.49-4.62 (m, 1H), 7.05-7.23 (m, 3H), 7.46-7.65 (m, 3H), 7.71-9.61 (m, 5H), 9.69 (s, 1H), 10.49 (s, 1H). ¹H NMR recorded at 80° C. |

### General Procedure X-8

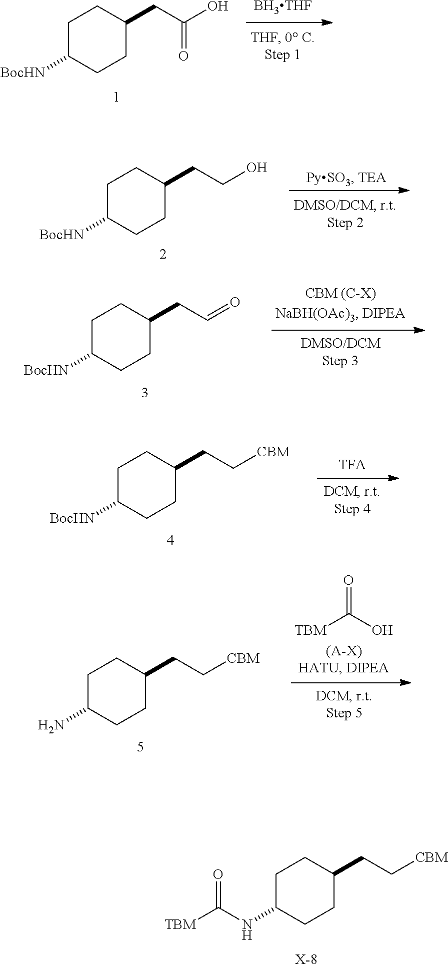

Step 1. Preparation of tert-Butyl N-[4-(2-hydroxyethyl)cyclohexyl]carbamate (2): A solution of 1 (1 eq.) in THF (0.38 M) was cooled to 0° C. and 1M BH₃ in THF (2.05 eq.) was added dropwise. The mixture was allowed to warm to room temperature. Upon completely consumption of starting material. Diluted aqueous HCl (0.5 N) was added (approx. 1 eq. relative to the starting material) and the mixture was stirred for 5 min at 0° C. EtOAc was added, then the phases were separated and the aqueous phase was extracted 3 times with EtOAc. Combined organic layers were washed 2 times with saturated aqueous NaHCO₃, dried over magnesium sulfate, filtered and concentrated under reduced pressure to give 2.

Step 2′. Preparation of tert-Butyl N-[4-(2-oxoethyl)cyclohexyl]carbamate (3): A solution of Py·SO₃ (4.0 eq.) in DMSO (0.2 M) was added to a solution of 2 (1.0 eq.) with triethylamine (6.0 eq.) in DCM (0.2 M). The reaction mixture was stirred at room temperature. After 3 hours, additional Py·SO₃ (1.0 eq.) and triethylamine (2.0 eq.) may be added and the mixture was allowed to stir for 2 additional hours. Brine and EtOAc were added to the mixture. Phases were separated and the aqueous phase was extracted 3 times with EtOAc. Combined organic layers were washed 2 times with half-brine and once with brine, then dried over magnesium sulfate and evaporated under reduced pressure. The residue was dry packed over silica gel and purified by normal phase chromatography (heptanes/EtOAc). Pure fractions were combined, evaporated under reduced pressure and dried under high vacuum to give 3.

Step 3. Preparation of (4): CBM C-X (1.0 eq.) was dissolved with triethylamine (12 eq.) in DCM (0.1 M), then 3 (1.1 eq.) was added to the mixture. DMSO (1 mL) was added to the suspension to help solubility. After 5 minutes of stirring, NaBH(OAc)₃ (3.0 eq.) was added to the reaction mixture. After 30 minutes, reaction was quenched with citric acid and water. The mixture was extracted three times with EtOAc. Combined organic layers were dried over Na₂SO₄ and purified to afford 4.

Step 4. Preparation of (5): Trifluoroacetic acid (54 eq.) was added to a solution of 4 (1.0 eq.) in DCM (0.1 M). After 15 minutes, volatiles were removed under reduced pressure. Coevaporation with toluene/MeCN, then with MeCN afford 5.

Step 4. Preparation of final product (X-8): To a solution of 5 (1.2 eq.) in DMF (0.2 M), DIPEA (10.0 eq.) was added. After 5 minutes TBM A-X (1.0 eq.) was added. After 5 minutes HATU (1.5 eq.) was added to the mixture. The reaction was stirred at room temperature for 1 hour. Material was purified by reverse phase chromatography and preparative HPLC purification. Pure fractions were collected, volatile evaporated, the compound was frozen and lyophilised to afford X-8.

### Example S47. 6-(5-Cyano-1H-pyrazolo[3,4-b]pyridin-1-yl)-4-(((R)-1-cyanoethyl)amino)-N— ((1r,4r)-4-(2-(4-(4-(2,6-dioxopiperidin-3-yl)phenyl)piperazin-1-yl)ethyl)cyclohexyl)nicotinamide (P-91)

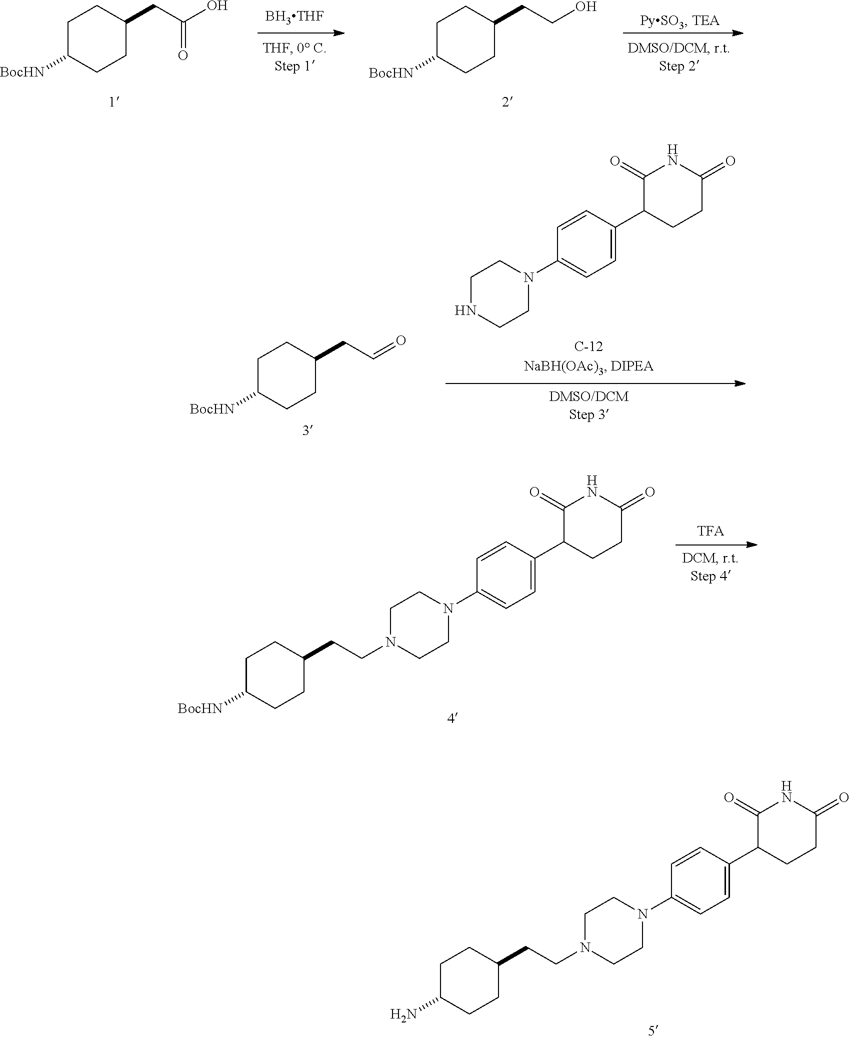

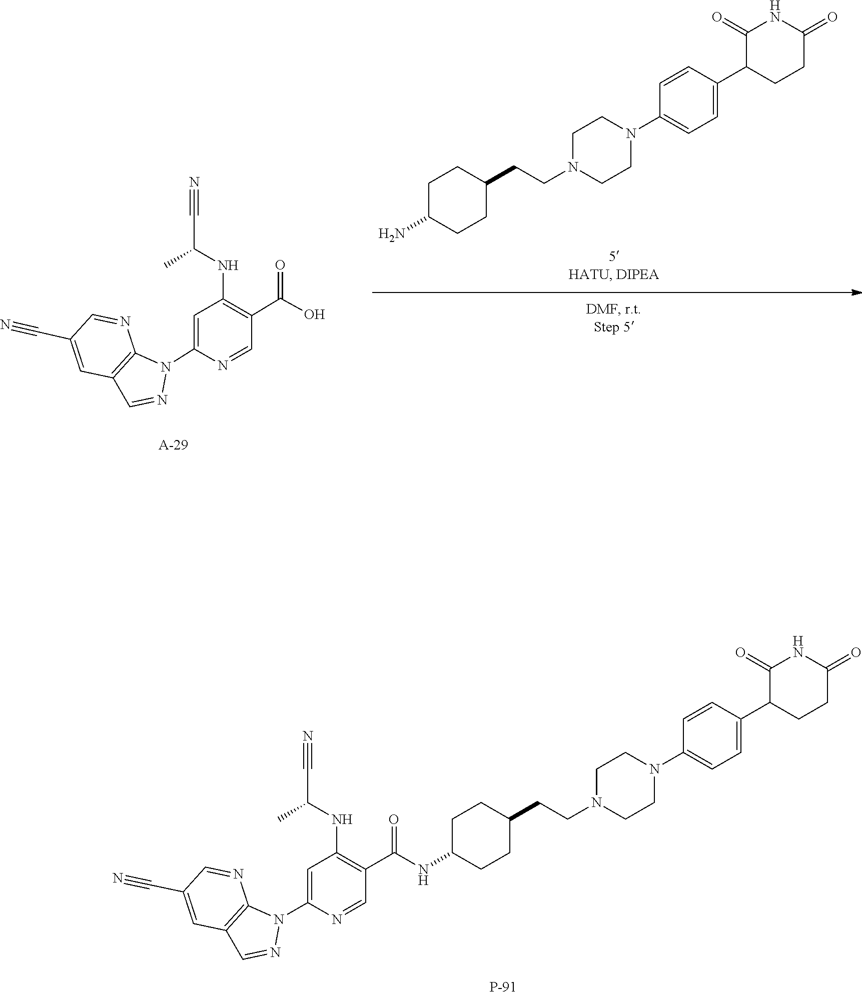

Step 1′. Preparation of tert-Butyl N-[4-(2-hydroxyethyl)cyclohexyl]carbamate (2′): A solution of 1′ (10.0 g, 38.86 mmol, 1 eq.) in THF (100 mL, 0.38 M) was cooled to 0° C. and 1M BH₃ in THF (80.0 mL, 80 mmol, 2.05 eq.) was added dropwise. The mixture was allowed to warm to room temperature over 2 h. TLC (heptanes/EtOAc) indicates that the starting material has been completely consumed. Diluted aqueous HCl (0.5 N) was added (approx. 1 eq. relative to the starting material) and the mixture was stirred for 5 min at 0° C. EtOAc was added, then the phases were separated and the aqueous phase was extracted 3 times with EtOAc. Combined organic layers were washed 2 times with saturated aqueous NaHCO₃, dried over magnesium sulfate, filtered and concentrated under reduced pressure to give 2′ (7.98 g, 82% yield) as a white solid.

¹H NMR (400 MHz, DMSO-d6) δ ppm 0.81-0.96 (m, 3H), 1.03-1.17 (m, 2H), 1.27-1.32 (m, 2H), 1.36 (s, 9H), 1.63-1.77 (m, 4H), 3.07-3.18 (m, 1H), 3.36-3.44 (m, 2H), 4.28 (t, J=5.1 Hz, 1H), 6.65 (br d, J=7.8 Hz, 1H).

Step 2′. Preparation of tert-Butyl N-[4-(2-oxoethyl)cyclohexyl]carbamate (3′): A solution of Py·SO₃ (18.05 g, 113.4 mmol, 4.0 eq.) in DMSO (70 mL, 0.2 M) was added to a solution of 2′ (6.9 g, 28.4 mmol, 1.0 eq.) with triethylamine (23.2 mL, 170.1 mmol, 6.0 eq.) in DCM (70 mL, 0.2 M). The reaction mixture was stirred at room temperature. After 3 h, Py·SO₃ (4.5 g, 28.36 mmol, 1.0 eq.) was added, along with triethylamine (7.73 mL, 56.71 mmol, 2.0 eq.) and the mixture was allowed to stir for 2 additional hours. Brine and EtOAc were added to the mixture. Phases were separated and the aqueous phase was extracted 3 times with EtOAc. Combined organic layers were washed 2 times with half-brine and once with brine, then dried over magnesium sulfate and evaporated under reduced pressure. The residue was dry packed over silica gel and purified by normal phase chromatography (heptanes/EtOAc). Pure fractions were combined, evaporated under reduced pressure and dried under high vacuum to give 3′ (4.85 g, 69%) as a white solid and was used as-is for next step.

¹H NMR (400 MHz, CDCl₃) δ ppm 1.05-1.20 (m, 4H), 1.44 (s, 9H), 1.72-1.91 (m, 3H), 1.99-2.06 (m, 2H), 2.33 (dd, J=6.6, 2.0 Hz, 2H), 3.38 (br d, J=2.9 Hz, 1H), 4.38 (br d, J=0.7 Hz, 1H), 9.76 (t, J=1.8 Hz, 1H).

Step 3′. Preparation of tert-Butyl N-[4-[2-[4-[4-(2,6-dioxo-3-piperidyl)phenyl]piperazin-1-yl]ethyl]cyclohexyl]carbamate (4′): C-12 (115.06 mg, 0.310 mmol, 1.0 eq.) was dissolved with TEA (0.52 mL, 3.72 mmol, 12 eq.) in DCM (3.098 mL, 0.1 M), then 3′ (82.24 mg, 0.340 mmol, 1.1 eq.) was added to the mixture. DMSO (1 mL) was added to the suspension to help solubility. After 5 minutes of stirring, NaBH(OAc)₃ (196.98 mg, 0.930 mmol, 3.0 eq.) was added to the reaction mixture. After 30 minutes, reaction was quenched with citric acid and water. The mixture was extracted three times with EtOAc. Combined organic layers were dried over Na₂SO₄ and dry packed over silica gel. Purification by normal phase flash chromatography (MeOH/DCM) afford 4′ (119 mg, 76% yield) as a white solid.

LCMS method 1: retention time: 1.457 min, 75.3% purity at 215 nm, [M+H]⁺=499.3.

¹H NMR (400 MHz, DMSO-d6) δ ppm 0.84-1.00 (m, 3H), 1.03-1.20 (m, 4H), 1.30-1.40 (m, 10H), 1.68-1.78 (m, 4H), 1.97-2.04 (m, 1H), 2.09-2.17 (m, 1H), 2.28-2.32 (m, 1H), 2.42-2.49 (m, 5H), 2.58-2.65 (m, 1H), 3.05-3.12 (m, 4H), 3.13-3.19 (m, 1H), 3.72 (dd, J=11.1, 5.3 Hz, 1H), 6.66 (d, J=7.8 Hz, 1H), 6.88 (d, J=9.0 Hz, 2H), 7.05 (d, J=9.0 Hz, 2H), 10.77 (s, 1H).

Step 4′. Step 9. Preparation of 3-[4-[4-[2-(4-Aminocyclohexyl)ethyl]piperazin-1-yl]phenyl]piperidine-2,6-dione;2,2,2-trifluoroacetic acid (5′): Trifluoroacetic acid (1.0 mL, 13.1 mmol, 54 eq.) was added to a solution of 4′ (119. mg, 0.240 mmol, 1.0 eq.) in DCM (2.0 mL, 0.1 M). After 15 minutes, volatiles were removed under reduced pressure. Coevaporation with toluene/MeCN, then with MeCN afford 5′ (122 mg, 99% yield).

LCMS method 1: retention time: 0.225 min, 99.9% purity at 215 nm, [M−TFA+H]⁺=399.3.

¹H NMR (400 MHz, DMSO-d6) δ ppm 0.95-1.07 (m, 2H), 1.21-1.34 (m, 3H), 1.53-1.63 (m, 2H), 1.77 (br d, J=11.7 Hz, 2H), 1.88-1.96 (m, 2H), 1.97-2.04 (m, 1H), 2.09-2.21 (m, 1H), 2.59-2.71 (m, 1H), 2.92-3.01 (m, 3H), 3.06-3.21 (m, 4H), 3.58 (br d, J=11.5 Hz, 2H), 3.72-3.87 (m, 3H), 6.97 (d, J=8.6 Hz, 2H), 7.11 (d, J=8.6 Hz, 2H), 7.83 (br d, J=3.9 Hz, 3H), 9.71-9.81 (m, 1H), 10.79 (s, 1H).

Step 4′. Preparation of 6-(5-Cyano-1H-pyrazolo[3,4-b]pyridin-1-yl)-4-4(R)-1-cyanoethyl)amino)-N-((1r,4r)-4-(2-(4-(4-(2,6-dioxopiperidin-3-yl)phenyl)piperazin-1-yl)ethyl)cyclohexyl)nicotinamide (P-91): To a solution of 3-[4-[4-[2-(4-aminocyclohexyl)ethyl]piperazin-1-yl]phenyl]piperidine-2,6-dione hydrochloride 5′ (140.9 mg, 0.324 mmol, 1.2 eq.) in DMF (1.3 mL, 0.2 M), DIPEA (0.47 mL, 2.700 mmol, 10.0 eq.) was added. After 5 minutes 4-[[(1R)-1-cyanoethyl]amino]-6-(5-cyanopyrazolo[3,4-b]pyridin-1-yl)pyridine-3-carboxylic acid A-29 (90.0 mg, 0.270 mmol, 1.0 eq.) was added. After 5 minutes HATU (154.0 mg, 0.410 mmol, 1.5 eq.) was added to the mixture. The reaction was stirred at room temperature for 1 hour. To obtain a pure product were performed as follows: purification by reverse phase chromatography (50 g C18 isco gold column, liquid deposit (DMF), elution: 5% MeCN/0.1% HCOOH over 4 CV, then 5% to 50% MeCN/0.1% HCOOH over 15 CV); followed by purification by reverse phase chromatography (50 g C18 isco gold column, liquid deposit (DMF), elution: 5% MeOH/0.1% HCOOH over 4 CV, then 5% to 50% MeOH/0.1% HCOOH over 15 CV); and a preparative HPLC purification. Pure fractions were collected, volatile evaporated, the compound was frozen and lyophilised to afford 6-(5-Cyano-1H-pyrazolo[3,4-b]pyridin-1-yl)-4-(((R)-1-cyanoethyl)amino)-N-((1r,4r)-4-(2-(4-(4-(2,6-dioxopiperidin-3-yl)phenyl)piperazin-1-yl)ethyl)cyclohexyl)nicotinamide P-91 (53.4 mg, 27% yield) as a light yellow solid.

LCMS method 2: retention time: 2.294 min, 99.9% purity at 215 nm, [M+2H]²⁺=357.7, [M+H]⁺=714.4.

¹H NMR (400 MHz, DMSO-d6) δ ppm 0.96-1.12 (m, 2H), 1.19-1.48 (m, 5H), 1.67 (d, J=6.8 Hz, 3H), 1.75-1.84 (m, 2H), 1.86-1.96 (m, 2H), 1.97-2.05 (m, 1H), 2.07-2.19 (m, 1H), 2.30-2.41 (m, 2H), 2.42-2.49 (m, 2H), 2.56-2.69 (m, 1H), 3.06-3.14 (m, 4H), 3.20-3.55 (m, 2H), 3.67-3.81 (m, 2H), 4.94 (quin, J=7.0 Hz, 1H), 6.89 (d, J=8.6 Hz, 2H), 7.05 (d, J=8.6 Hz, 2H), 7.48 (s, 1H), 8.54 (d, J=7.8 Hz, 1H), 8.70 (d, J=7.3 Hz, 2H), 8.77 (d, J=7.3 Hz, 1H), 9.00-9.08 (m, 2H), 10.76 (s, 1H).

Table 37 summarizes the compounds prepared using General Procedure X-8.

TABLE 37
Final Compounds Prepared via General Procedure X-8

| Compound | TMB | CBM | | |
|---|---|---|---|---|
| No. | Portion | Portion | Structure | Characterization |
| P-35 | A7 | C12 | 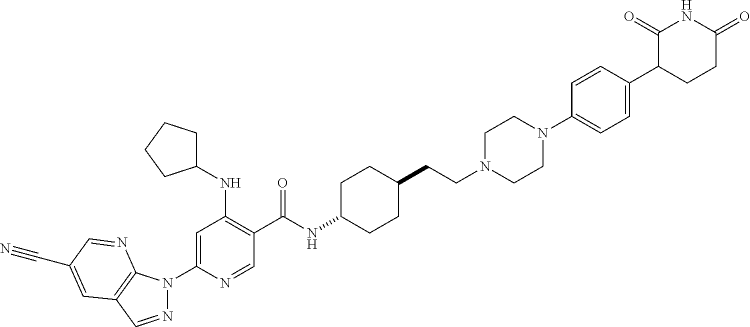 | 52 % yield as a tan solid. LCMS method 1: retention time: 2.465 min, 99.9% purity at 215 nm, [M − HCOOH + 2H]²⁺ = 365.3; [M − HCOOH + H]⁺ = 729.4 ¹H NMR (400 MHz, DMSO-d₆) δ ppm 1.03 (q, J = 12.2 Hz, 2H), 1.20-1.47 (m, 6H), 1.46-1.56 (m, 2H), 1.57-1.74 (m, 4H), 1.80 (br. d, J = 12.0 Hz, 2H), 1.87 (br. d, J = 10.8 Hz, 2H), 1.96-2.21 (m, 4H), 2.42-2.47 (m, 2H), 2.56-2.69 (m, 5H), 3.10-3.19 (m, 4H), 3.73 (br. dd, J = 10.9, 4.8 Hz, 2H), 3.90 (sxt, J = 6.4 Hz, 1H), 6.90 (d, J = 8.8 Hz, 2H), 7.06 (d, J = 8.6 Hz, 2H), 7.33 (s, 1H), 8.14 (s, 2H), 8.37 (d, J = 7.8 Hz, 1H), 8.59-8.66 (m, 3H), 9.02 (d, J = 2.0 Hz, 1H), 9.05 (d, J = 2.0 Hz, 1H), 10.77 (s, 1H), 12.24-13.05 (s, 2H). |
| P-48 | A4 | C12 | 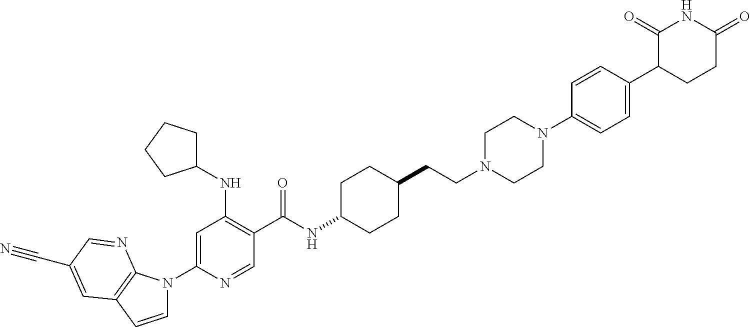 | 33% yield as an white solid. LCMS method 2: retention time: 2.794 min, 97.2% purity at 215 nm, [M − FA + H]⁺ = 728.3, [M − FA+2H]²⁺ = 364.8. ¹H NMR (400 MHz, DMSO-d₆) δ ppm 0.95-1.16 (m, 3H), 1.20-1.44 (m, 6H), 1.49-1.60 (m, 2H), 1.61-1.90 (m, 9H), 1.96-2.04 (m, 1H), 2.08-2.19 (m, 3H), 2.33-2.39 (m, 2H), 2.41-2.46 (m, 2H), 2.57-2.66 (m, 1H), 3.07-3.14 (m, 4H), 3.68-3.77 (m, 2H), 3.87-3.96 (m, 1H), 6.85-6.92 (m, 3H), 7.05 (d, J = 8.6 Hz, 2H), 8.11-8.19 (m, 2H), 8.29 (br d, J = 7.6 Hz, 1H), 8.53 (d, J = 3.9 Hz, 1H), 8.56 (s, 1H), 8.67 (d, J = 2.0 Hz, 1H), 8.70 (br d, J = 6.1 Hz, 1H), 8.83 (d, J = 1.7 Hz, 1H), 10.76 (s, 1H). Two proton signals overlap with solvent peak by ¹H NMR. One proton was not apparent by ¹H NMR. |
| P-49 | A14 | C12 | 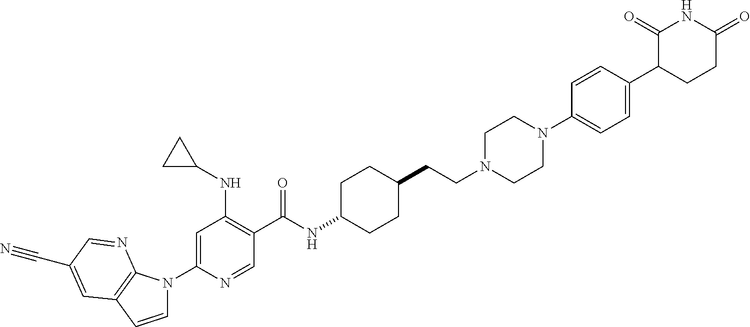 | 42% yield as an off-white solid. LCMS method 2: retention time: 2.614 min, 99.3% purity at 215 nm, [M + H]⁺ = 700.3, [M + 2H]²⁺ = 350.6. ¹H NMR (400 MHz, DMSO-d₆) δ ppm 0.55-0.62 (m, 2H), 0.89-0.96 (m, 2H), 0.96-1.09 (m, 2H), 1.21-1.44 (m, 6H), 1.75-1.90 (m, 4H), 1.96-2.05 (m, 1H), 2.08-2.19 (m, 1H), 2.32-2.38 (m, 2H), 2.42-2.49 (m, 3H), 2.55-2.68 (m, 3H), 3.06-3.15 (m, 4H), 3.66-3.77 (m, 2H), 6.85-6.92 (m, 3H), 7.05 (br d, J = 8.8 Hz, 2H), 8.31 (br d, J = 7.6 Hz, 1H), 8.53-8.59 (m, 3H), 8.62 (s, 1H), 8.68 (d, J = 2.0 Hz, 1H), 8.83 (d, J = 2.0 Hz, 1H), 10.76 (s, 1H). |
| P-50 | A8 | C12 | 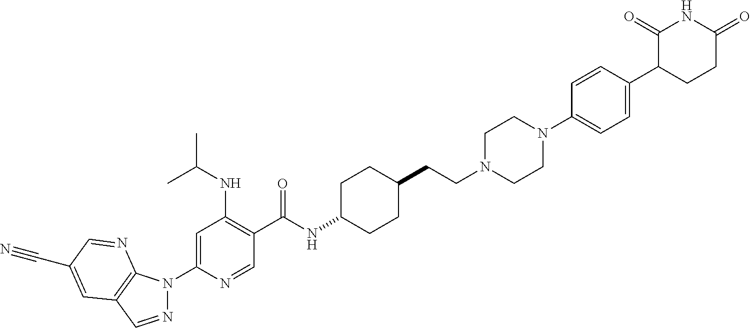 | 17 % yield as a white solid. LCMS method 2: retention time: 2.288 min, 99.1% purity at 215 nm, [M + H]⁺ = 703.4, [M + 2H]²⁺ = 352.3. ¹H NMR (400 MHz, DMSO-d₆) δ ppm 0.96-1.12 (m, 3H), 1.24 (d, J = 6.1 Hz, 6H), 1.27-1.45 (m, 5H), 1.77-1.92 (m, 4H), 1.95-2.20 (m, 3H), 2.40-2.47 (m, 3H), 2.53-2.65 (m, 3H), 3.12 (br s, 4H), 3.68-3.81 (m, 3H), 6.89 (d, J = 8.6 Hz, 2H), 7.05 (d, J = 8.6 Hz, 2H), 7.29 (s, 1H), 8.36 (d, J = 8.1 Hz, 1H), 8.52 (d, J = 7.3 Hz, 1H), 8.59 (s, 1H), 8.63 (s, 1H), 9.01 (d, J = 2.0 Hz, 1H), 9.04 (d, J = 2.0 Hz, 1H), 10.77 (s, 1H) Two protons were not apparent by ¹H NMR. |
| P-51 | A10 | C12 | 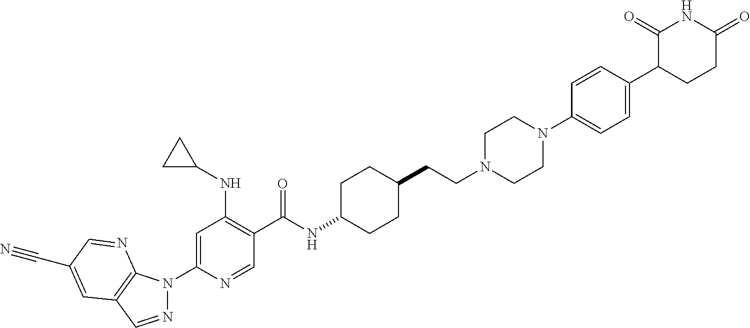 | 76 % yield as a white solid. LCMS method 2: retention time: 2.328 min, 99.4% purity at 215 nm, [M + 2H]²⁺ = 351.3, [M + H]⁺ = 701.6. ¹H NMR (400 MHz, DMSO-d6) δ ppm 0.53-0.59 (m, 2H), 0.81-0.88 (m, 2H), 0.93-1.08 (m, 2H), 1.20-1.44 (m, 6H), 1.74-1.91 (m, 4H), 1.96-2.05 (m, 1H), 2.06-2.19 (m, 1H), 2.30-2.38 (m, 2H), 2.41-2.49 (m, 3H), 2.53-2.72 (m, 3H), 3.07-3.14 (m, 4H), 3.67-3.77 (m, 2H), 6.88 (d, J = 8.6 Hz, 2H), 7.05 (d, J = 8.6 Hz, 2H), 7.67 (s, 1H), 8.38 (d, J = 7.8 Hz, 1H), 8.56 (s, 1H), 8.59 (s, 1H), 8.65 (s, 1H), 9.02 (d, J = 2.0 Hz, 1H), 9.06 (d, J = 2.0 Hz, 1H), 10.77 (s, 1H). |
| P-52 | A7 | C24 | 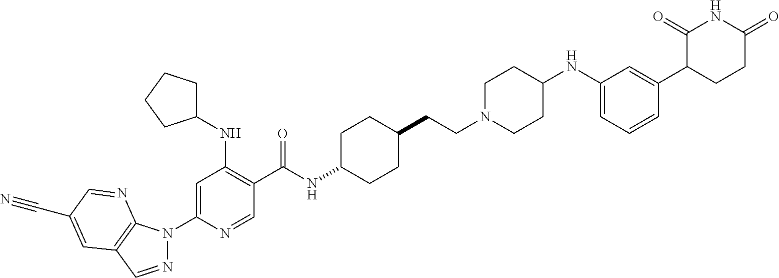 | 26% yield as a white solid. LCMS method 2: retention time: 2.328 min, 99.4% purity at 215 nm, [M + 2H]²⁺ = 351.3, [M + H]⁺ = 701.6. ¹H NMR (400 MHz, DMSO-d6) δ ppm 0.96-1.08 (m, 2H), 1.19-1.44 (m, 7H), 1.46-1.56 (m, 2H), 1.57-1.74 (m, 4H), 1.74-1.82 (m, 2H), 1.84-1.92 (m, 3H), 2.00-2.15 (m, 5H), 2.34-2.48 (m, 4H), 2.56-2.66 (m, 1H), 2.83-2.93 (m, 2H), 3.12-3.24 (m, 2H), 3.65-3.78 (m, 2H), 3.85-3.94 (m, 1H), 5.45 (br d, J = 6.8 Hz, 1H), 6.34 (br d, J = 7.8 Hz, 1H), 6.40 (s, 1H), 6.43-6.49 (m, 1H), 7.00 (t, J = 7.8 Hz, 1H), 7.33 (s, 1H), 8.36 (br d, J = 7.8 Hz, 1H), 8.60 (s, 1H), 8.62-8.66 (m, 2H), 9.02 (d, J = 2.2 Hz, 1H), 9.06 (d, J = 2.0 Hz, 1H), 10.78 (s, 1H). |
| P-56 | A7 | C25 | 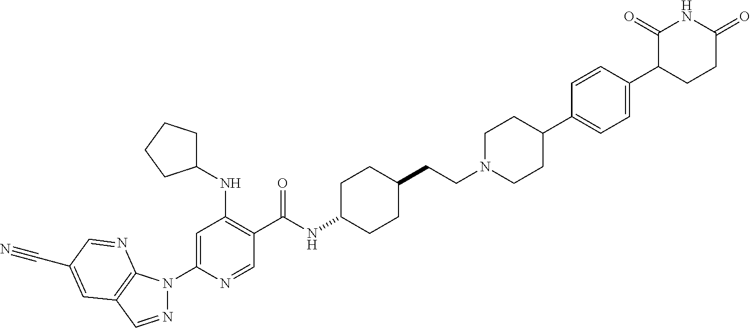 | 43% yield as a white solid. LCMS method 2: retention time: 2.494 min, 99.9% purity at 215 nm, [M + H]⁺ = 728.5, [M + 2H]²⁺ = 364.8. ¹H NMR (400 MHz, DMSO-d₆) δ ppm 0.95-1.08 (m, 2H), 1.21-1.44 (m, 6H), 1.46-1.55 (m, 2H), 1.57-1.90 (m, 13H), 1.94-2.10 (m, 5H), 2.11-2.23 (m, 1H), 2.31-2.38 (m, 2H), 2.60-2.70 (m, 1H), 2.98 (br d, J = 11.2 Hz, 2H), 3.68-3.78 (m, 1H), 3.81 (dd, J = 11.4, 4.8 Hz, 1H), 3.85-3.94 (m, 1H), 7.13 (d, J = 8.3 Hz, 2H), 7.21 (d, J = 8.1 Hz, 2H), 7.33 (s, 1H), 8.36 (d, J = 7.6 Hz, 1H), 8.60 (s, 1H), 8.63-8.66 (m, 2H), 9.02 (d, J = 2.0 Hz, 1H), 9.05 (d, J = 2.0 Hz, 1H), 10.81 (s, 1H). One proton was not apparent by ¹H NMR. |
| P-57 | A7 | C26 | 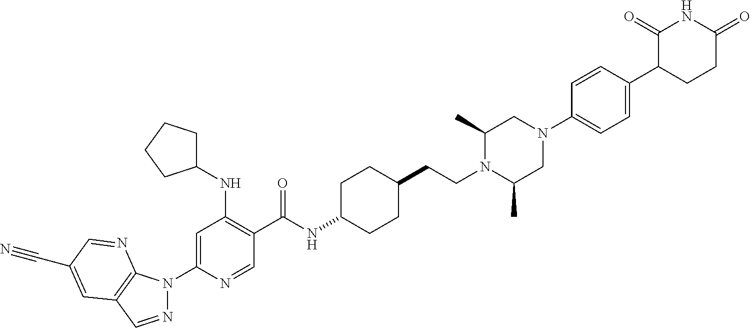 | 41% yield as a white solid. LCMS method 2: retention time: 2.525 min, 97.9% purity at 215 nm, [M + H]⁺ = 757.4, [M + 2H]²⁺ = 379.3. ¹H NMR (400 MHz, DMSO-d6) δ ppm 0.99-1.19 (m, 9H), 1.25-1.40 (m, 4H), 1.47-1.55 (m, 2H), 1.59-1.73 (m, 4H), 1.79 (d, J = 11.0 Hz, 2H), 1.87 (d, J = 11.1 Hz, 2H), 1.98-2.16 (m, 4H), 2.32 (t, J = 10.9 Hz, 2H), 2.42-2.47 (m, 1H), 2.58-2.77 (m, 5H), 3.51 (d, J = 10.5 Hz, 2H), 3.72 (dd, J = 10.9, 5.0 Hz, 2H), 3.85-3.94 (m, 1H), 6.87 (d, J = 8.3 Hz, 2H), 7.03 (d, J = 8.6 Hz, 2H), 7.33 (s, 1H), 8.37 (d, J = 7.6 Hz, 1H), 8.58-8.67 (m, 3H), 9.03 (d, J = 13.0 Hz, 2H), 10.76 (s, 1H). |
| P-59 | A7 | C28 | 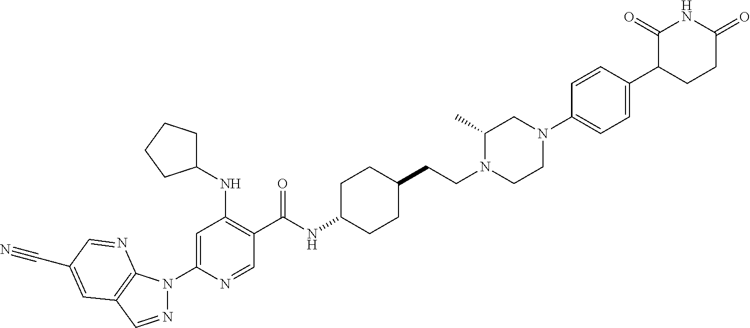 | 41% yield as a white solid. LCMS method 2: retention time: 2.486 min, 98.8% purity at 215 nm, [M + H]⁺ = 743.5, [M + 2H]²⁺ = 372.3. ¹H NMR (400 MHz, DMSO-d6) δ ppm 0.93-1.10 (m, 2H), 1.14-1.46 (m, 6H), 1.46-1.57 (m, 2H), 1.57-1.76 (m, 4H), 1.76-1.90 (m, 4H), 1.95-2.08 (m, 3H), 2.37 (t, J = 7.0 Hz, 2H), 2.53-2.64 (m, 2H), 2.82-2.95 (m, 1H), 3.30-3.65 (m, 5H), 3.67-3.78 (m, 2H), 3.89 (m, 2H), 5.07 (dd, J = 13.0, 5.4 Hz, 1H), 7.20-7.30 (m, 1H), 7.33 (s, 2H), 7.67 (d, J = 8.6 Hz, 1H), 8.35 (d, J = 7.6 Hz, 1H), 8.55-8.67 (m, 3H), 9.00 (d, J = 1.8 Hz, 1H), 9.04 (d, J = 1.7 Hz, 1H), 11.08 (s, 1H). |
| P-61 | A7 | C2 | 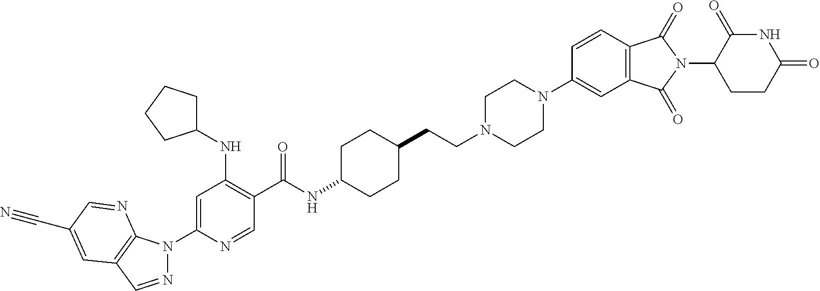 | 10% yield as a yellow solid. LCMS method 2: retention time: 2.451 min, 99.9% purity at 215 nm, [M + H]⁺ = 798.5, [M + 2H]²⁺ = 399.8.'H NMR (400 MHz, DMSO- d6) δ ppm 0.93-1.10 (m, 2H), 1.14-1.46 (m, 6H), 1.46-1.57 (m, 2H), 1.57-1.76 (m, 4H), 1.76-1.90 (m, 4H), 1.95-2.08 (m, 3H), 2.37 (t, J = 7.0 Hz, 2H), 2.53-2.64 (m, 2H), 2.82-2.95 (m, 1H), 3.30-3.65 (m, 5H), 3.67-3.78 (m, 2H), 3.89 (m, 2H), 5.07 (dd, J = 13.0, 5.4 Hz, 1H), 7.20-7.30 (m, 1H), 7.33 (s, 2H), 7.67 (d, J = 8.6 Hz, 1H), 8.35 (d, J = 7.6 Hz, 1H), 8.55-8.67 (m, 3H), 9.00 (d, J = 1.8 Hz, 1H), 9.04 (d, J = 1.7 Hz, 1H), 11.08 (s, 1H). |
| P-62 | A7 | C30 | 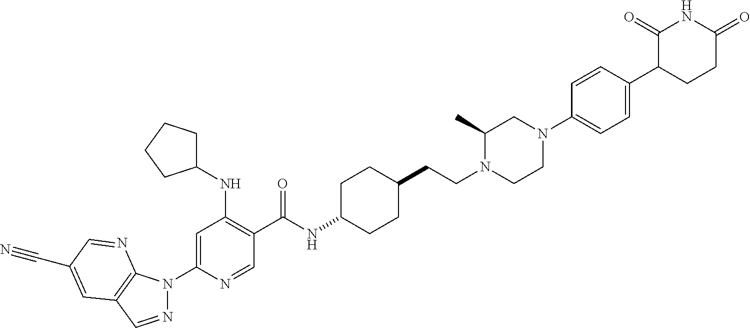 | 26.8% yield as an off-white solid. LCMS method 2: retention time: 2.492 min. 98.7% purity at 215 nm, [M + H]⁺ = 743.5, [M + 2H]²⁺ = 372.3. ¹H NMR (400 MHz, DMSO-d6) δ ppm 0.97-1.13 (m, 5H), 1.21-1.43 (m, 5H), 1.45-1.57 (m, 2H), 1.59-1.74 (m, 4H), 1.75-1.91 (m, 4H), 1.94-2.19 (m, 5H), 2.23-2.39 (m, 2H), 2.42-2.46 (m, 1H), 2.57-2.68 (m, 2H), 2.74-2.85 (m, 2H), 2.87-2.96 (m, 1H), 3.37-3.47 (m, 2H), 3.69-3.78 (m, 2H), 3.85-3.94 (m, 1H), 6.88 (d, J = 8.6 Hz, 2H), 7.05 (d, J = 8.6 Hz, 2H), 7.33 (s, 1H), 8.36 (d, J = 7.8 Hz, 1H), 8.58-8.67 (m, 3H), 9.01 (d, J = 2.0 Hz, 1H), 9.05 (d, J = 2.0 Hz, 1H), 10.77 (s, 1H). |
| P-63 | A16 | C12 | 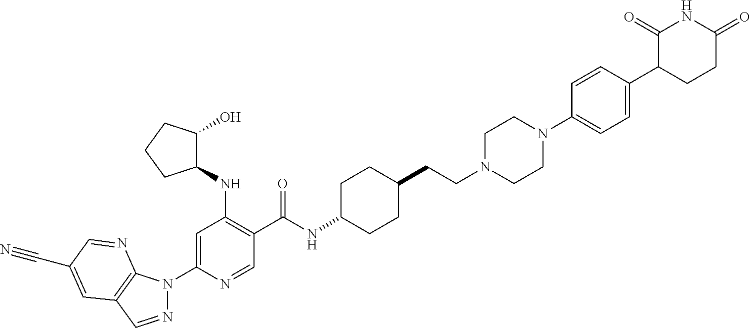 | 58.9% yield as a white solid. LCMS method 2: retention time: 2.158 min. 99.9% purity at 215 nm, [M + H]⁺ = 745.4, [M + 2H]²⁺ = 373.2 ¹H NMR (400 MHz, DMSO-d6) δ ppm 0.96-1.10 (m, 3H), 1.21-1.59 (m, 9H), 1.63-1.91 (m, 8H), 1.95-2.05 (m, 1H), 2.10-2.27 (m, 2H), 2.31-2.38 (m, 2H), 2.40-2.46 (m, 1H), 2.62 (s, 1H), 3.07-3.14 (m, 4H), 3.54-3.68 (m, 1H), 3.68-3.79 (m, 2H), 3.89-3.95 (m, 1H), 4.99 (d, J = 4.2 Hz, 1H), 6.88 (d, J = 8.8 Hz, 2H), 7.05 (d, J = 8.8 Hz, 2H), 7.37 (s, 1H), 8.37 (d, J = 7.8 Hz, 1H), 8.60 (s, 1H), 8.62-8.67 (m, 2H), 9.01 (d, J = 2.0 Hz, 1H), 9.04 (d, J = 2.0 Hz, 1H), 10.77 (s, 1H). |
| P-64 | A17 | C12 | 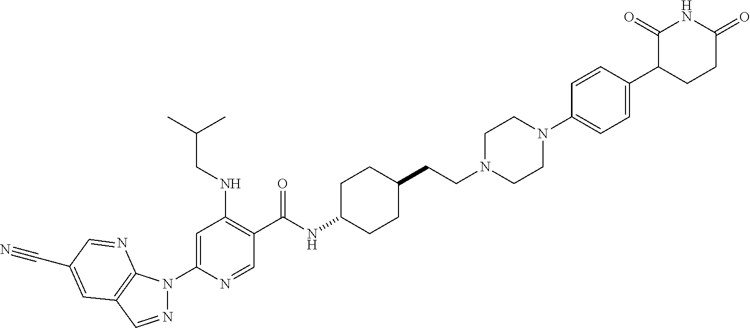 | 18% yield as a light yellow solid. LCMS method 2: retention time: 2.423 min. 95.2% purity at 215 nm, [M + H]⁺ = 717.4, [M + 2H]²⁺ = 359.3 ¹H NMR (400 MHz, DMSO-d6) δ ppm 0.90-0.95 (m, 1H), 0.97 (d, J = 6.6 Hz, 6H), 1.01-1.11 (m, 2H), 1.19-1.45 (m, 6H), 1.76-2.04 (m, 6H), 2.06-2.20 (m, 1H), 2.36-2.46 (m, 2H), 2.53-2.70 (m, 4H), 3.03-3.10 (m, 2H), 3.11-3.21 (m, 4H), 3.69-3.80 (m, 2H), 6.89 (d, J = 8.8 Hz, 2H), 7.05 (d, J = 8.8 Hz, 2H), 7.29 (s, 1H), 8.37 (d, J = 7.8 Hz, 1H), 8.58 (s, 1H), 8.63 (s, 1H), 8.64-8.70 (m, 1H), 9.01 (d, J = 2.2 Hz, 1H), 9.04 (d, J = 2.0 Hz, 1H), 10.77 (s, 1H). |
| P-65 | A18 | C12 | 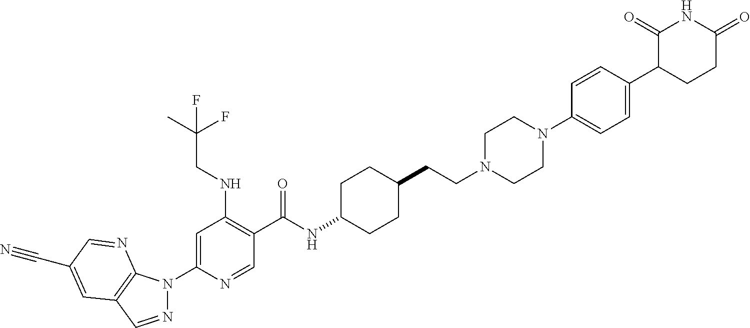 | 23% yield as a white solid. LCMS method 2: retention time: 2.364 min. 99.1% purity at 215 nm, [M + H]⁺ = 739.4, [M + 2H]²⁺ = 370.3 ¹H NMR (400 MHz, DMSO-d6) δ ppm 0.97-1.12 (m, 2H), 1.18-1.48 (m, 7H), 1.69 (t, J = 19.1 Hz, 3H), 1.81 (d, J = 10.5 Hz, 2H), 1.89 (d, J = 10.5 Hz, 2H), 1.95-2.06 (m, 1H), 2.07-2.20 (m, 1H), 2.36 (t, J = 7.5 Hz, 2H), 2.41-2.46 (m, 1H), 2.56-2.70 (m, 2H), 3.06-3.15 (m, 4H), 3.29 (s, 1H), 3.68-3.90 (m, 4H), 6.89 (d, J = 8.6 Hz, 2H), 7.05 (d, J = 8.6 Hz, 2H), 7.42 (s, 1H), 8.45 (d, J = 7.6 Hz, 1H), 8.63 (s, 1H), 8.65 (s, 1H), 8.86 (t, J = 6.2 Hz, 1H), 9.02 (d, J = 2.0 Hz, 1H), 9.04 (d, J = 1.7 Hz, 1H), 10.77 (s, 1H). ¹⁹NMR (377 MHz, DMSO-d6) δ ppm −94.54 (s, 2F). |
| P-66 | A19 | C12 | 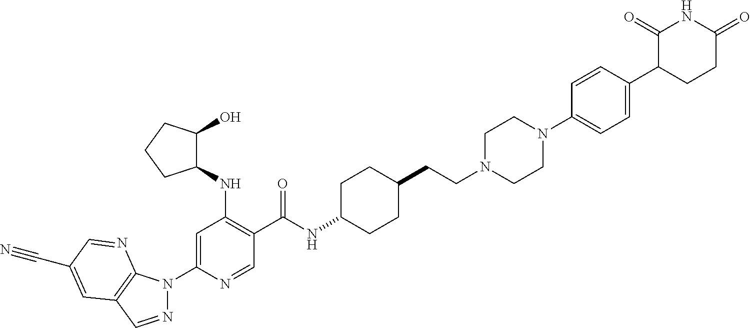 | 15% yield as an off white solid. LCMS method 2: retention time: 2.165 min. 97.4% purity at 215 nm, [M + H]⁺ = 745.5, [M + 2H]²⁺ = 373.3 ¹H NMR (400 MHz, DMSO-d6) δ ppm 0.98-1.09 (m, 2H), 1.26-1.43 (m, 6H), 1.45-1.67 (m, 4H), 1.74-1.91 (m, 6H), 1.95-2.05 (m, 2H), 2.07-2.16 (m, 2H), 2.31-2.38 (m, 2H), 2.40-2.45 (m, 1H), 2.58-2.68 (m, 2H), 3.07-3.15 (m, 4H), 3.65-3.76 (m, 3H), 4.08-4.15 (m, 1H), 5.09 (d, J = 4.2 Hz, 1H), 6.89 (d, J = 8.6 Hz, 2H), 7.05 (d, J = 8.6 Hz, 2H), 7.27 (s, 1H), 8.29 (d, J = 7.3 Hz, 1H), 8.55 (s, 1H), 8.63 (s, 1H), 8.83 (d, J = 6.4 Hz, 1H), 9.01 (d, J = 2.0 Hz, 1H), 9.04 (d, J = 1.7 Hz, 1H), 10.77 (s, 1H). |
| P-67 | A7 | C31 | 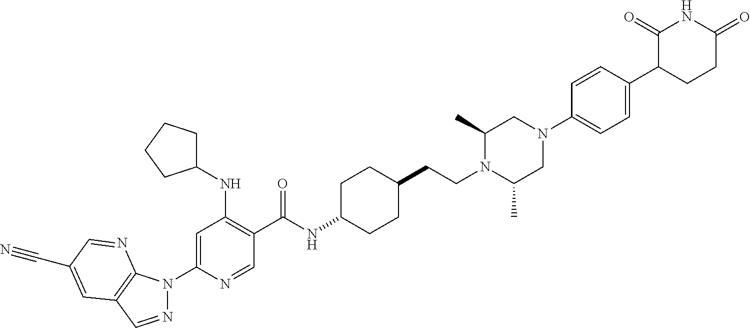 | 13% yield as a white solid. LCMS method 2: retention time: 2.165 min. 99.9 % purity at 215 nm, [M + H]⁺ = 757.5, [M + 2H]²⁺ = 379.3 ¹H NMR (400 MHz, DMSO-d6) δ ppm 1.02 (s, 3H), 1.03 (s, 3H), 1.27-1.37 (m, 4H), 1.45-1.55 (m, 2H), 1.59-1.75 (m, 5H), 1.76-1.91 (m, 4H), 1.96-2.15 (m, 4H), 2.22-2.32 (m, 1H), 2.42-2.48 (m, 2H), 2.61-2.76 (m, 2H), 2.85 (dd, J = 11.2, 6.4 Hz, 2H), 2.92-3.03 (m, 3H), 3.06-3.16 (m, 2H), 3.68-3.77 (m, 2H), 3.86-3.93 (m, 1H), 6.87 (d, J = 8.6 Hz, 2H), 7.03 (d, J = 8.3 Hz, 2H), 7.33 (s, 1H), 8.36 (d, J = 7.6 Hz, 1H), 8.60 (s, 1H), 8.62-8.66 (m, 2H), 9.01 (d, J = 2.0 Hz, 1H), 9.05 (d, J = 2.0 Hz, 1H), 10.76 (s, 1H). |
| P-68 | A7 | C32 | 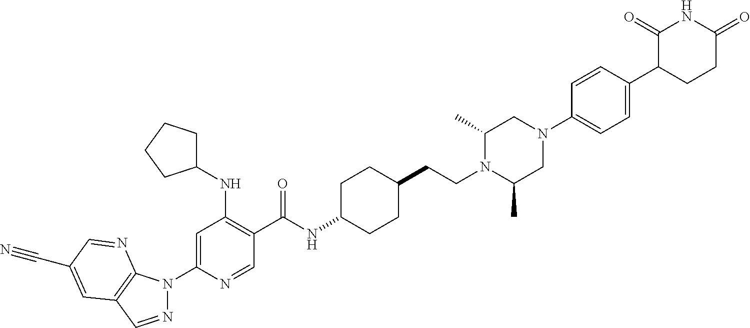 | 17% yield as a white solid. LCMS method 2: retention time: 2.597 min. 99.4 % purity at 215 nm, [M + H]⁺ = 757.5, [M + 2H]²⁺ = 379.3 ¹H NMR (400 MHz, DMSO-d6) δ ppm 1.02 (s, 3H), 1.03 (s, 3H), 1.27-1.41 (m, 5H), 1.45-1.55 (m, 2H), 1.58-1.74 (m, 4H), 1.76-1.91 (m, 4H), 1.94-2.19 (m, 5H), 2.22-2.32 (m, 1H), 2.41-2.48 (m, 2H), 2.57-2.77 (m, 2H), 2.85 (dd, J = 10.8, 6.1 Hz, 2H), 2.91-3.05 (m, 2H), 3.08-3.15 (m, 2H), 3.67-3.77 (m, 2H), 3.86-3.94 (m, 1H), 6.87 (d, J = 8.3 Hz, 2H), 7.03 (d, J = 8.8 Hz, 2H), 7.33 (s, 1H), 8.36 (d, J = 7.8 Hz, 1H), 8.60 (s, 1H), 8.62-8.67 (m, 2H), 9.01 (d, J = 1.7 Hz, 1H), 9.05 (d, J = 2.0 Hz, 1H), 10.76 (s, 1H). |
| P-70 | A20 | C12 | 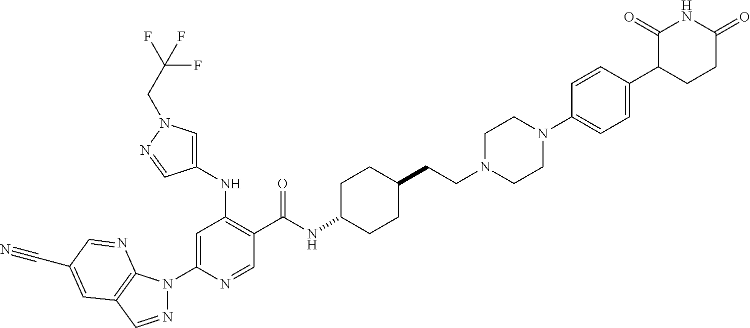 | 11% yield as a light yellow solid. LCMS method 4: retention time: 2.394 min. 96.0 % purity at 215 nm, [M + H]⁺ = 809.3, [M + 2H]²⁺ = 405.3 ¹H NMR (400 MHz, DMSO-d6) δ ppm 1.00-1.17 (m, 3H), 1.19-1.29 (m, 3H), 1.32-1.46 (m, 3H), 1.46-1.60 (m, 2H), 1.76-1.96 (m, 4H), 1.96-2.05 (m, 1H), 2.08-2.22 (m, 1H), 2.40-2.46 (m, 1H), 2.59-2.70 (m, 2H), 2.93-3.20 (m, 4H), 3.49-3.67 (m, 1H), 3.69-3.87 (m, 2H), 5.17 (q, J = 9.2 Hz, 2H), 6.93 (d, J = 7.8 Hz, 2H), 7.08 (d, J = 8.1 Hz, 2H), 7.62 (s, 1H), 7.74 (s, 1H), 8.05-8.10 (m, 1H), 8.56 (d, J = 7.8 Hz, 1H), 8.63 (s, 1H), 8.72 (s, 1H), 9.00-9.03 (m, 2H), 10.02 (s, 1H), 10.78 (s, 1H). ¹⁹F NMR (377 MHz, DMSO-d6) δ ppm −70.23 (s, 3F). |
| P-71 | A21 | C12 | 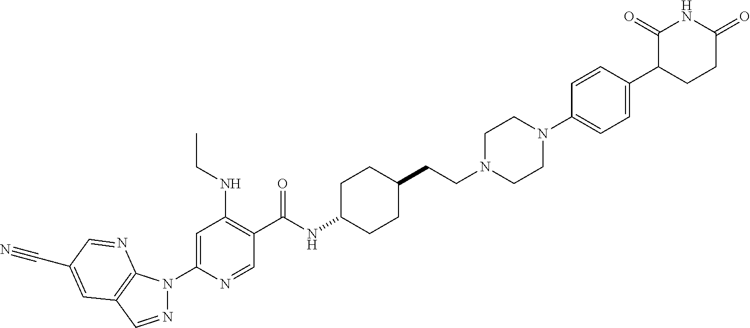 | 23% yield as a white solid. LCMS method 4: retention time: 2.206 min. 99.9 % purity at 215 nm, [M + H]⁺ = 689.3, [M + 2H]²⁺ = 345.3 ¹H NMR (400 MHz, DMSO-d6) δ ppm 0.98-1.15 (m, 2H), 1.24 (m, 4H), 1.28-1.42 (m, 3H), 1.42-1.54 (m, 2H), 1.74-1.94 (m, 4H), 1.95-2.06 (m, 1H), 2.06-2.22 (m, 1H), 2.41-2.47 (m, 1H), 2.58-2.69 (m, 3H), 2.70-2.90 (m, 3H), 3.10-3.22 (m, 3H), 3.23-3.29 (m, 3H), 3.68-3.81 (m, 2H), 6.91 (d, J = 8.6 Hz, 2H), 7.07 (d, J = 8.3 Hz, 2H), 7.27 (s, 1H), 8.34-8.47 (m, 2H), 8.57 (s, 1H), 8.63 (s, 1H), 9.01 (d, J = 2.0 Hz, 1H), 9.04 (d, J = 2.0 Hz, 1H), 10.78 (s, 1H). |
| P-72 | A10 | C25 | 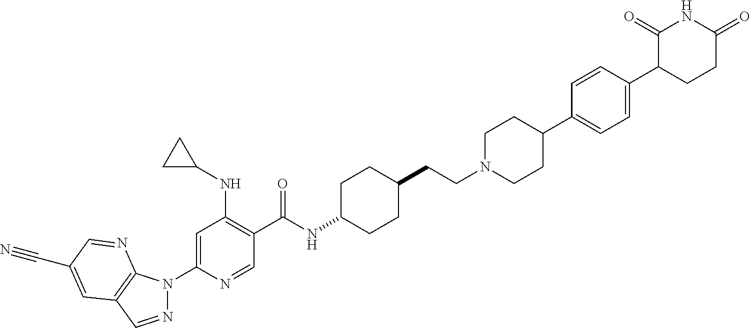 | 28% yield as an off-white solid. LCMS method 2: retention time: 2.379 min. 99.9 % purity at 215 nm, [M − HCOOH + H]⁺ = 700.4, [M − HCOOH + 2H]²⁺ = 350.8 ¹H NMR (400 MHz, DMSO-d6) δ ppm 0.51-0.61 (m, 2H), 0.85 (q, J = 5.2 Hz, 2H), 1.02 (q, J = 11.7 Hz, 2H), 1.20-1.47 (m, 6H), 1.58-1.70 (m, 2H), 1.78 (t, J = 13.9 Hz, 4H), 1.87 (d, J = 11.0 Hz, 2H), 1.97-2.09 (m, 3H), 2.10-2.23 (m, 1H), 2.39 (br t, J = 7.1 Hz, 2H), 2.55-2.72 (m, 3H), 3.01 (d, J = 10.3 Hz, 2H), 2.97-2.98 (m, 1H), 3.67-3.77 (m, 1H), 3.81 (dd, J = 11.6, 4.8 Hz, 1H), 7.14 (d, J = 8.1 Hz, 2H), 7.21 (d, J = 8.6 Hz, 2H), 7.67 (s, 1H), 8.18 (s, 1H), 8.38 (d, J = 7.8 Hz, 1H), 8.55 (s, 1H), 8.60 (s, 1H), 8.66 (s, 1H), 9.02 (d, J = 2.0 Hz, 1H), 9.06 (d, J = 1.7 Hz, 1H), 10.81 (s, 1H). |
| P-73 | A1 | C12 | 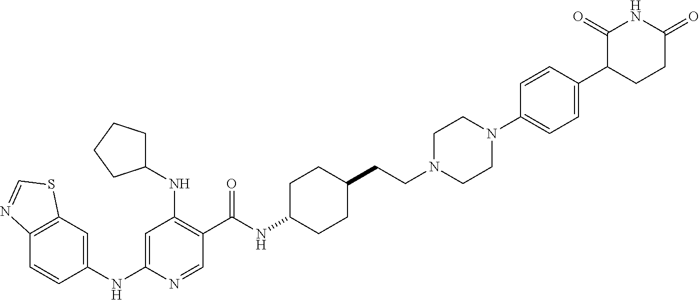 | 34% yield as an white solid. LCMS method 2: retention time: 2.140min. 99.0 % purity at 215 nm, [M + H]⁺ = 735.4, [M + 2H]²⁺ = 368.2 ¹H NMR (400 MHz, DMSO-d6) δ ppm 0.92-1.08 (m, 2H), 1.15-1.51 (m, 8H), 1.55-1.73 (m, 4H), 1.73-1.87 (m, 4H), 1.93-2.06 (m, 3H), 2.07-2.20 (m, 1H), 2.32-2.41 (m, 2H), 2.42-2.48 (m, 2H), 2.52-2.55 (m, 2H), 2.57-2.69 (m, 1H), 3.05-3.18 (m, 4H), 3.59-3.78 (m, 3H), 6.02 (s, 1H), 6.88 (d, J = 8.6 Hz, 2H), 7.05 (d, J = 8.6 Hz, 2H), 7.55 (dd, J = 8.9, 1.8 Hz, 1H), 7.93 (d, J = 8.8 Hz, 1H), 7.97 (d, J = 7.7 Hz, 1H), 8.33-8.40 (m, 2H), 8.69 (d, J = 1.7 Hz, 1H), 9.14 (s, 1H), 9.23 (s, 1H), 10.77 (s, 1H). Two protons signals obscured by the solvent peak in ¹H NMR. |
| P-74 | A22 | C12 | 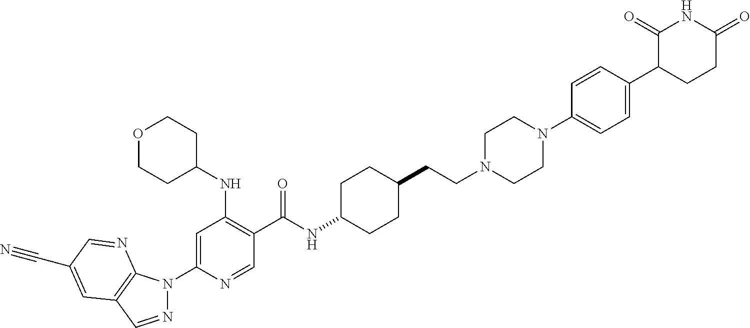 | 36% yield as an white solid. LCMS method 2: retention time: 2.219 min, 99.9 % purity at 215 nm, [M + H]⁺ = 745.4, [M + 2H]²⁺ = 373.4 ¹H NMR (400 MHz, DMSO-d6) δ ppm 0.96-1.09 (m, 2H), 1.21-1.55 (m, 8H), 1.75-1.91 (m, 4H), 1.93-2.05 (m, 3H), 2.06-2.17 (m, 1H), 2.30-2.38 (m, 2H), 2.41-2.45 (m, 1H), 2.56-2.68 (m, 2H), 3.05-3.15 (m, 4H), 3.48 (t, J = 10.3 Hz, 2H), 3.68-3.78 (m, 3H), 3.82-3.89 (m, 2H), 6.88 (d, J = 8.6 Hz, 2H), 7.04 (d, J = 8.8 Hz, 2H), 7.37 (s, 1H), 8.38 (d, J = 7.8 Hz, 1H), 8.60-8.65 (m, 2H), 8.66 (d, J = 7.8 Hz, 1H), 9.01 (d, J = 2.0 Hz, 1H), 9.04 (d, J = 2.0 Hz, 1H), 10.76 (s, 1H). Two protons were not apparent by ¹H NMR. |
| P-75 | A23 | C12 | 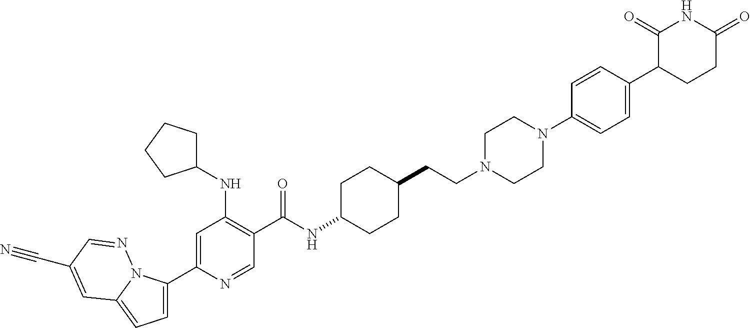 | 22% yield as a yellow solid. LCMS method 2: retention time: 2.192 min, 99.9% purity at 215 nm, [M + H]⁺ = 728.4, [M + 2H]²⁺ = 364.8 ¹H NMR (400 MHz, DMSO-d6) δ ppm 1.02 (q, J = 12.2 Hz, 3H), 1.19-1.45 (m, 6H), 1.52 (dd, J = 11.6, 5.7 Hz, 2H), 1.61-1.75 (m, 4H), 1.75-1.90 (m, 4H), 1.95-2.04 (m, 1H), 2.06-2.19 (m, 3H), 2.26-2.40 (m, 3H), 2.41-2.46 (m, 1H), 2.57-2.75 (m, 2H), 3.06-3.15 (m, 4H), 3.64-3.77 (m, 2H), 3.91 (dq, J = 11.9, 5.7 Hz, 1H), 6.88 (d, J = 8.6 Hz, 2H), 7.05 (d, J = 8.6 Hz, 2H), 7.10 (d, J = 4.9 Hz, 1H), 7.83 (d, J = 4.9 Hz, 1H), 8.11 (s, 1H), 8.25 (d, J = 7.6 Hz, 1H), 8.57 (d, J = 6.1 Hz, 1H), 8.67 (s, 1H), 8.74 (d, J = 2.0 Hz, 1H), 8.83 (d, J = 2.2 Hz, 1H), 10.77 (s, 1H). |
| P-76 | A11 | C12 | 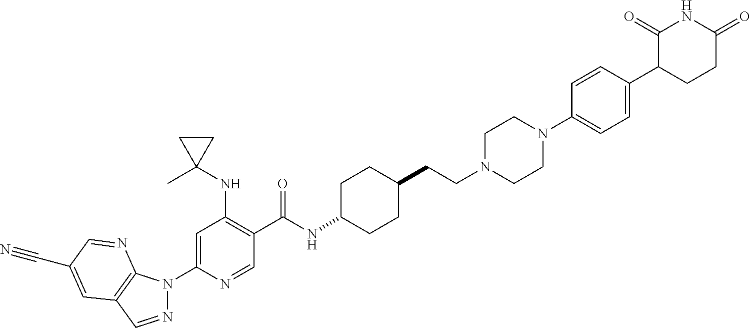 | 19% yield as an off-white solid. LCMS method 2: retention time: 2.380 min, 99.0% purity at 215 nm, [M + H]⁺ = 715.3, [M + 2H]²⁺ = 358.3 ¹H NMR (400 MHz, DMSO-d6) δ ppm 0.79 (s, 3H), 0.96-1.08 (m, 2H), 1.21-1.44 (m, 8H), 1.75-1.93 (m, 4H), 1.93-2.24 (m, 3H), 2.35 (t, J = 7.2 Hz, 2H), 2.39-2.46 (m, 1H), 2.47-2.48 (m, 2H), 2.58-2.67 (m, 1H), 3.03-3.17 (m, 4H), 3.72 (dd, J = 10.6, 4.8 Hz, 2H), 6.88 (d, J = 8.6 Hz, 2H), 7.04 (d, J = 8.6 Hz, 2H), 7.67 (s, 1H), 8.36 (d, J = 7.6 Hz, 1H), 8.60 (s, 1H), 8.66 (s, 1H), 8.72 (s, 1H), 9.02 (d, J = 1.7 Hz, 1H), 9.06 (d, J = 1.7 Hz, 1H), 10.77 (s, 1H). Two protons were not apparent by ¹H NMR. |
| P-77 | A24 | C12 | 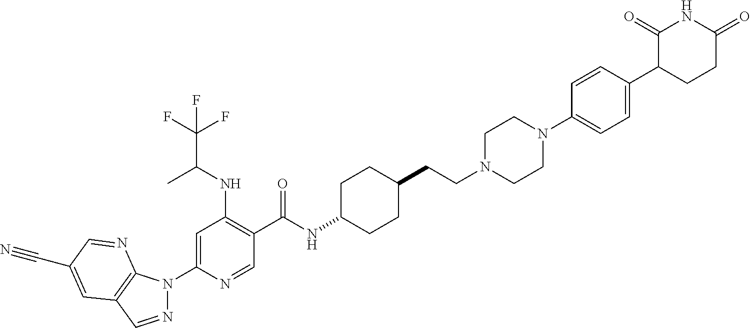 | 40% yield as a white solid. LCMS method 2: retention time: 2.527 min, 99.9% purity at 215 nm, [M + H]⁺ = 757.3, [M + 2H]²⁺ = 379.2 ¹H NMR (400 MHz, DMSO-d6) δ ppm 0.94-1.11 (m, 3H), 1.15-1.48 (m, 9H), 1.73-1.93 (m, 4H), 1.95-2.05 (m, 1H), 2.09-2.21 (m, 1H), 2.28-2.40 (m, 2H), 2.41-2.46 (m, 1H), 2.56-2.70 (m, 1H), 3.06-3.18 (m, 4H), 3.66-3.83 (m, 2H), 4.71-4.86 (m, 1H), 6.89 (d, J = 8.8 Hz, 2H), 7.05 (d, J = 8.8 Hz, 2H), 7.53 (s, 1H), 8.52 (d, J = 7.58 Hz, 1H), 8.66 (s, 1H), 8.71 (s, 1H), 8.89-9.15 (m, 3H), 10.77 (s, 1H). ¹⁹NMR (377 MHz, DMSO-d6) δ ppm −75.92 (s, 3F) Two protons obscured by the solvent peak in ¹H NMR. |
| P-78 | A25 | C12 | 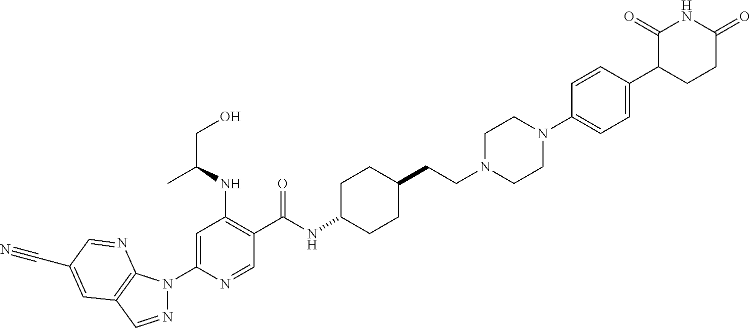 | 21% yield as a white solid. LCMS method 2: retention time: 2.058 min, 99.9% purity at 215 nm, [M + H]⁺ = 719.4, [M + 2H]²⁺ = 360.3 ¹H NMR (400 MHz, DMSO-d6) δ ppm 0.95-1.49 (m, 12H), 1.75-1.93 (m, 4H), 1.95-2.06 (m, 1H), 2.06-2.21 (m, 1H), 2.31-2.39 (m, 2H), 2.41-2.46 (m, 1H), 2.54-2.71 (m, 2H), 3.05-3.19 (m, 4H), 3.40-3.55 (m, 3H), 3.61-3.81 (m, 3H), 4.96 (br. s, 1H), 6.89 (d, J = 8.6 Hz, 2H), 7.05 (d, J = 8.6 Hz, 2H), 7.29 (s, 1H), 8.34 (d, J = 7.8 Hz, 1H), 8.58 (s, 1H), 8.61-8.66 (m, 2H), 9.01 (d, J = 1.7 Hz, 1H), 9.04 (d, J = 1.7 Hz, 1H), 10.77 (s, 1H). |
| P-79 | A10 | C34 | 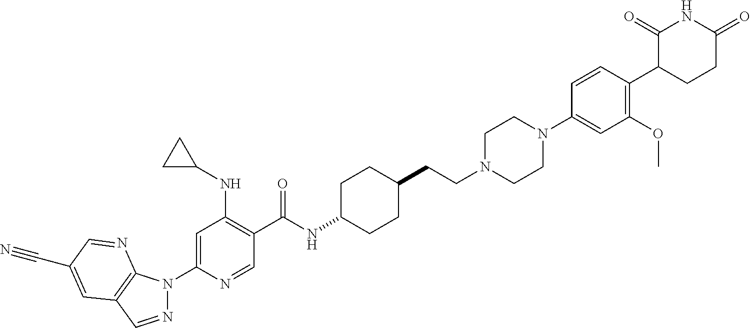 | 39% yield as an off-white solid. LCMS method 2: retention time: 2.343min. 99.1% purity at 215 nm, [M + H]⁺ = 731.4, [M + 2H]²⁺ = 366.2 ¹H NMR (400 MHz, DMSO-d6) δ ppm 0.53-0.59 (m, 2H), 0.82-0.90 (m, 2H), 0.97-1.09 (m, 2H), 1.22-1.44 (m, 6H), 1.76-1.92 (m, 5H), 2.04-2.23 (m, 2H), 2.28-2.44 (m, 4H), 2.54-2.70 (m, 3H), 3.08-3.18 (m, 4H), 3.28-3.30 (m, 1H), 3.72 (s, 3H), 3.75-3.82 (m, 1H), 6.45 (d, J = 8.6 Hz, 1H), 6.54 (s, 1H), 6.92 (d, J = 8.3 Hz, 1H), 7.67 (s, 1H), 8.38 (d, J = 7.6 Hz, 1H), 8.55 (s, 1H), 8.59 (s, 1H), 8.66 (s, 1H), 8.99-9.09 (m, 2H), 10.67 (s, 1H). |
| P-80 | A10 | C35 | 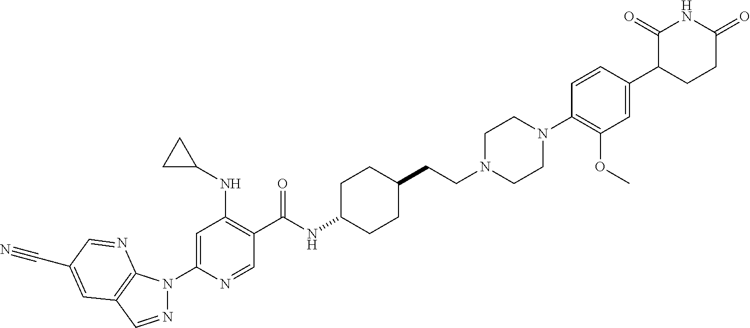 | 76% yield as a white solid. LCMS method 2: retention time: 2.325 min, 97.4% purity at 215 nm, [M + H]⁺ = 731.4, [M + 2H]²⁺ = 366.2 ¹H NMR (400 MHz, DMSO-d6) δ ppm 0.54-0.59 (m, 2H), 0.81-0.88 (m, 2H), 0.95-1.07 (m, 2H), 1.21-1.43 (m, 6H), 1.75-1.90 (m, 4H), 1.96-2.05 (m, 1H), 2.13-2.25 (m, 1H), 2.31-2.37 (m, 2H), 2.43-2.47 (m, 2H), 2.54-2.69 (m, 3H), 2.89-2.99 (m, 4H), 3.71-3.78 (m, 5H), 6.70 (d, J = 8.3 Hz, 1H), 6.77-6.85 (m, 2H), 7.67 (s, 1H), 8.37 (d, J = 7.6 Hz, 1H), 8.55 (s, 1H), 8.59 (s, 1H), 8.65 (s, 1H), 9.02 (d, J = 2.0 Hz, 1H), 9.05 (d, J = 2.0 Hz, 1H), 10.78 (s, 1H). One proton was not apparent by ¹H NMR. |
| P-82 | A26 | C12 | 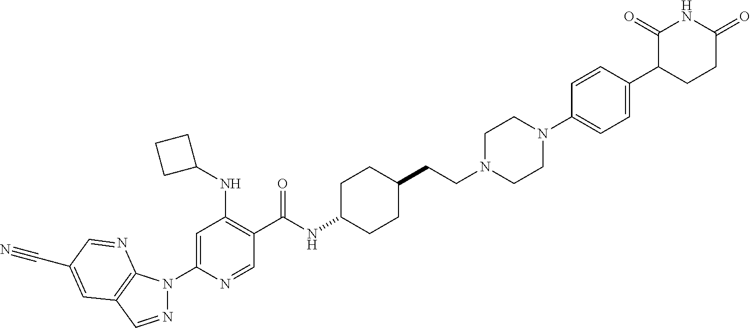 | 40% yield as a white solid. LCMS method 2: retention time: 2.418 min, 99.9% purity at 215 nm, [M + H]⁺ = 715.4, [M + 2H]²⁺ = 358.3 ¹H NMR (400 MHz, DMSO-d6) δ ppm 0.96-1.10 (m, 2H), 1.20-1.47 (m, 6H), 1.76-2.05 (m, 10H), 2.06-2.20 (m, 1H), 2.31-2.39 (m, 2H), 2.40-2.47 (m, 3H), 2.57-2.69 (m, 2H), 3.08-3.15 (m, 4H), 3.67-3.81 (m, 3H), 3.97-4.08 (m, 1H), 6.89 (d, J = 8.8 Hz, 2H), 7.05 (d, J = 8.6 Hz, 2H), 7.22 (s, 1H), 8.38 (d, J = 7.8 Hz, 1H), 8.60 (s, 1H), 8.63-8.68 (m, 2H), 9.01 (d, J = 2.0 Hz, 1H), 9.06 (d, J = 2.0 Hz, 1H), 10.77 (s, 1H). |
| P-83 | A12 | C12 | 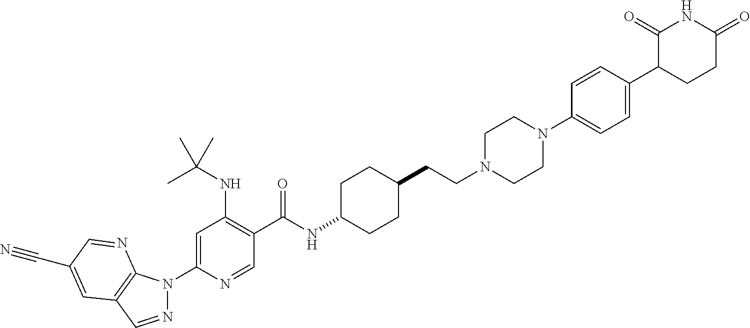 | 25% yield as a light yellow solid. LCMS method 2: retention time: 2.403 min, 99.9% purity at 215 nm, [M + H]⁺ = 717.5, [M + 2H]²⁺ = 359.3 1H NMR (400 MHz, DMSO-d6) δ ppm 0.97-1.11 (m, 3H), 1.21-1.42 (m, 6H), 1.44 (s, 9H), 1.76-1.91 (m, 4H), 1.97-2.05 (m, 1H), 2.07-2.20 (m, 1H), 2.31-2.39 (m, 2H), 2.41-2.48 (m, 2H), 2.57-2.69 (m, 2H), 3.07-3.15 (m, 4H), 3.73 (dd, J = 10.6, 5.0 Hz, 2H), 6.89 (d, J = 8.8 Hz, 2H), 7.05 (d, J = 8.6 Hz, 2H), 7.62 (s, 1H), 8.36 (d, J = 7.6 Hz, 1H), 8.58 (s, 1H), 8.64 (s, 1H), 8.88 (s, 1H), 9.02 (d, J = 2.0 Hz, 1H), 9.05 (d, J = 2.0 Hz, 1H), 10.77 (s, 1H). |
| P-84 | A10 | C36 | 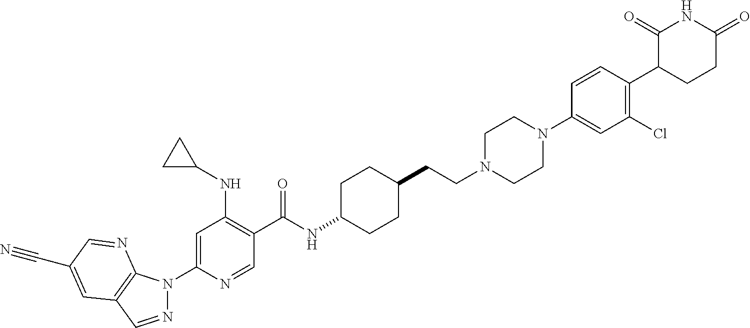 | 40% yield as an off-white solid. LCMS method 2: retention time: 2.396 min, 99.9% purity at 215 nm, [M + H]⁺ = 735.3, [M + 2H]²⁺ = 368.3 ¹H NMR (400 MHz, DMSO-d6) δ ppm 0.51-0.61 (m, 2H), 0.80-0.90 (m, 2H), 0.95-1.10 (m, 2H), 1.20-1.45 (m, 6H), 1.99 (br d, J = 2.0 Hz, 6H), 2.14-2.29 (m, 1H), 2.31-2.38 (m, 2H), 2.54-2.62 (m, 2H), 2.64-2.80 (m, 2H), 3.15 (br s, 4H), 3.65-3.79 (m, 1H), 4.05 (dd, J = 12.1, 5.0 Hz, 1H), 6.86-6.91 (m, 1H), 6.94 (d, J = 2.0 Hz, 1H), 7.12 (d, J = 8.6 Hz, 1H), 7.67 (s, 1H), 8.38 (d, J = 7.3 Hz, 1H), 8.55 (s, 1H), 8.59 (s, 1H), 8.65 (s, 1H), 9.02 (d, J = 2.0 Hz, 1H), 9.06 (d, J = 2.0 Hz, 1H), 10.83 (s, 1H). One proton was not apparent by ¹H NMR. |
| P-85 | A10 | C37 | 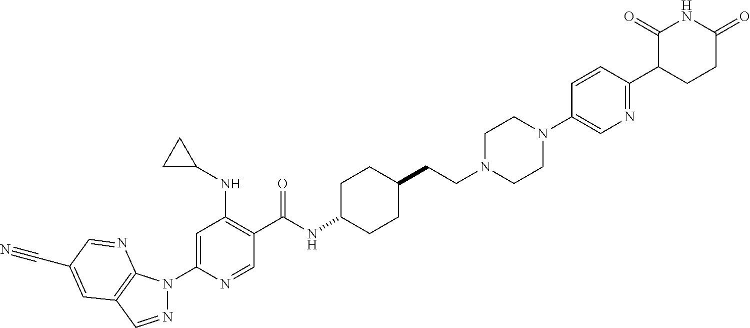 | 11% yield as a white solid. LCMS method 5: retention time: 2.547 min, 99.9% purity at 215 nm, [M + H]⁺ = 702.3, [M + 2H]²⁺ = 351.7 ¹H NMR (400 MHz, DMSO-d6) δ ppm 0.53-0.60 (m, 2H), 0.82-0.89 (m, 2H), 0.96-1.09 (m, 2H), 1.28-1.41 (m, 2H), 1.43-1.60 (m, 3H), 1.78-1.96 (m, 6H), 1.96-2.05 (m, 1H), 2.07-2.19 (m, 1H), 2.54-2.69 (m, 3H), 2.84-2.97 (m, 2H), 3.27-3.28 (m, 2H), 3.41-3.49 (m, 3H), 3.66-3.78 (m, 2H), 6.91-6.92 (m, 2H), 7.04-7.05 (m, 2H), 7.69 (s, 1H), 8.41 (d, J = 7.6 Hz, 1H), 8.55-8.62 (m, 2H), 8.66 (s, 1H), 9.03 (d, J = 1.7 Hz, 1H), 9.06 (d, J = 1.7 Hz, 1H), 10.77 (s, 1H). |
| P-86 | A27 | C12 | 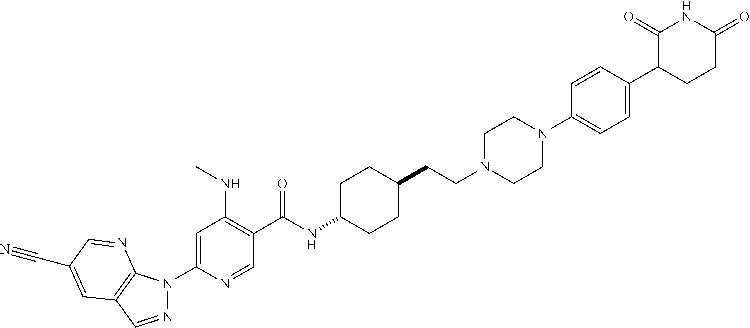 | 31% yield as an off-white solid. LCMS method 2: retention time: 2.098 min, 99.1% purity at 215 nm, [M + H]⁺ = 675.3, [M + 2H]²⁺ = 338.2 ¹H NMR (400 MHz, DMSO-d6) δ ppm 1.03 (qd, J = 12.7, 2.4 Hz, 2H), 1.23-1.46 (m, 5H), 1.80 (br d, J = 11.7 Hz, 2H), 1.84-1.92 (m, 2H), 1.94-2.06 (m, 1H), 2.07-2.20 (m, 1H), 2.36 (br t, J = 7.5 Hz, 2H), 2.41-2.49 (m, 2H), 2.63 (ddd, J = 17.1, 11.4, 5.5 Hz, 1H), 2.89 (d, J = 4.9 Hz, 3H), 3.11 (br t, J = 4.4 Hz, 4H), 3.67-3.80 (m, 2H), 6.89 (d, J = 8.8 Hz, 2H), 7.05 (d, J = 8.8 Hz, 2H), 7.23 (s, 1H), 8.29 (q, J = 4.6 Hz, 1H), 8.35 (d, J = 7.8 Hz, 1H), 8.53 (s, 1H), 8.63 (s, 1H), 9.01 (d, J = 2.0 Hz, 1H), 9.05 (d, J = 2.0 Hz, 1H), 10.77 (s, 1H). Three protons were not apparent by ¹H NMR. |
| P-89 | A28 | C12 | 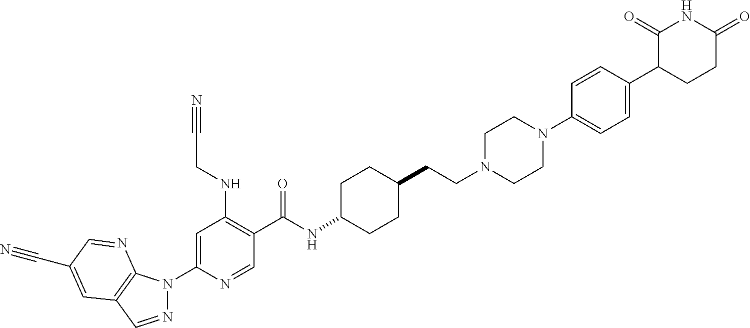 | 10% yield as a white solid. LCMS method 2: retention time: 2.193 min, 94.5% purity at 215 nm, [M + H]⁺ = 700.4, [M + 2H]²⁺ = 350.8 ¹H NMR (400 MHz, DMSO-d6) δ ppm 1.00-1.13 (m, 2H), 1.21-1.52 (m, 7H), 1.77-1.85 (m, 2H), 1.88-1.95 (m, 2H), 1.96-2.21 (m, 3H), 2.42-2.49 (m, 2H), 2.64 (ddd, J = 17.0, 11.4, 5.1 Hz, 4H), 3.08-3.22 (m, 3H), 3.70-3.79 (m, 2H), 4.55 (d, J = 6.4 Hz, 2H), 6.91 (d, J = 8.6 Hz, 2H), 7.07 (d, J = 8.6 Hz, 2H), 7.42 (s, 1H), 8.51 (d, J = 7.8 Hz, 1H), 8.60 (t, J = 6.4 Hz, 1H), 8.64 (s, 1H), 8.69 (s, 1H), 9.04 (d, J = 1.2 Hz, 2H), 10.78 (s, 1H). |
| P-90 | A10 | C39 | 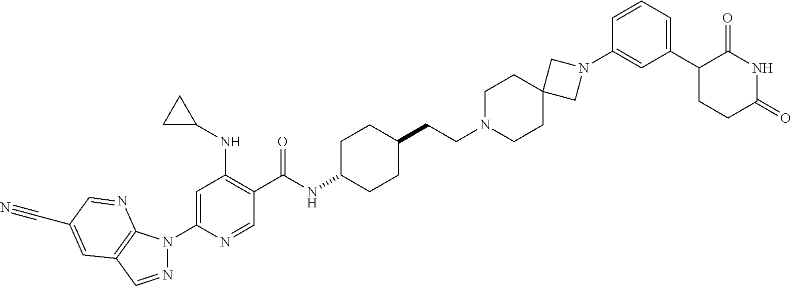 | 12% yield as a white solid. LCMS method 2: retention time: 2.394 min, 95.8% purity at 215 nm, [M + H]⁺ = 741.4, [M + 2H]²⁺ = 371.3 ¹H NMR (400 MHz, DMSO-d₆) δ ppm 0.52-0.59 (m, 2H), 0.82-0.89 (m, 2H), 0.96-1.09 (m, 3H), 1.18-1.52 (m, 7H), 1.72-1.92 (m, 8H), 1.99-2.17 (m, 3H), 2.45-2.48 (m, 1H), 2.55-2.68 (m, 4H), 3.47-3.58 (m, 4H), 3.68-3.79 (m, 2H), 6.23-6.26 (m, 1H), 6.29-6.34 (m, 1H), 6.48-6.53 (m, 1H), 7.11 (t, J = 7.8 Hz, 1H), 7.67 (s, 1H), 8.36-8.41 (m, 1H), 8.53-8.56 (m, 1H), 8.60 (s, 1H), 8.66 (s, 1H), 9.01-9.03 (m, 1H), 9.05-9.07 (m, 1H), 10.79 (s, 1H). |
| P-91 | A29 | C12 | 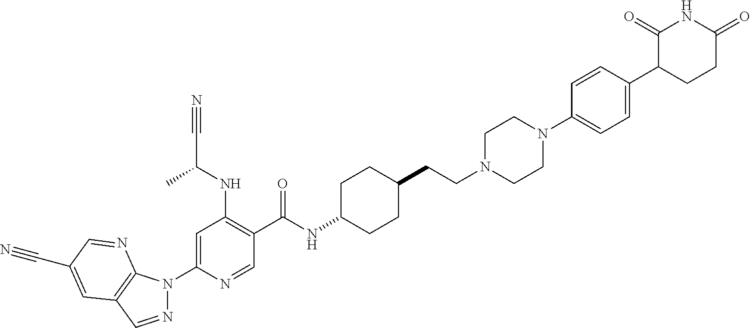 | 27 % yield as a white solid. LCMS method 2: retention time: 2.296 min, 99.9% purity at 215 nm, [M + 2H]²⁺ = 357.8, [M + H]⁺ = 714.3. ¹H NMR (400 MHz, DMSO-d₆) δ ppm 0.96-1.12 (m, 2H), 1.19-1.48 (m, 5H), 1.67 (d, J = 6.8 Hz, 3H), 1.75-1.84 (m, 2H), 1.86-1.96 (m, 2H), 1.97-2.05 (m, 1H), 2.07-2.19 (m, 1H), 2.30-2.41 (m, 2H), 2.42-2.49 (m, 2H), 2.56-2.69 (m, 1H), 3.06-3.14 (m, 4H), 3.20-3.55 (m, 2H), 3.67-3.81 (m, 2H), 4.94 (quin, J = 7.0 Hz, 1H), 6.89 (d, J = 8.6 Hz, 2H), 7.05 (d, J = 8.6 Hz, 2H), 7.48 (s, 1H), 8.54 (d, J = 7.8 Hz, 1H), 8.70 (d, J = 7.3 Hz, 2H), 8.77 (d, J = 7.3 Hz, 1H), 9.00-9.08 (m, 2H), 10.76 (s, 1H). |
| P-92 | A30 | C12 | 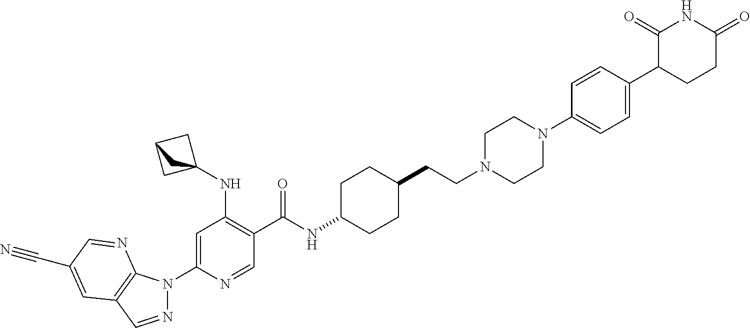 | 27 % yield as a light pink solid. LCMS method 2: retention time: 2.548 min, 99.9% purity at 215 nm, [M + 2H]²⁺ = 364.3, [M + H]⁺ = 727.4. ¹H NMR (400 MHz, DMSO-d₆) δ ppm 0.95-1.09 (m, 3H), 1.22-1.44 (m, 6H), 1.75-1.92 (m, 5H), 1.96-2.05 (m, 2H), 2.07-2.19 (m, 2H), 2.21 (s, 5H), 2.33-2.39 (m, 2H), 2.58 (s, 1H), 2.59-2.68 (m, 2H), 3.07-3.14 (m, 4H), 3.70-3.74 (m, 2H), 6.89 (d, J = 8.8 Hz, 2H), 7.05 (d, J = 8.8 Hz, 2H), 7.88 (s, 1H), 8.42 (d, J = 7.8 Hz, 1H), 8.63 (s, 1H), 8.66 (s, 1H), 8.99 (s, 1H), 9.03 (d, J = 2.0 Hz, 1H), 9.09 (d, J = 2.0 Hz, 1H), 10.77 (s, 1H). |
| P-93 | A31 | C12 | 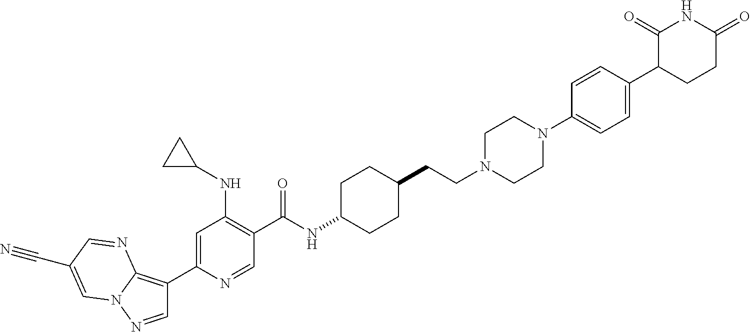 | 15 % yield as a yellow solid. LCMS method 2: retention time: 1.951 min, 95.8% purity at 215 nm, [M + 2H]²⁺ = 351.3, [M + H]⁺ = 701.4. ¹H NMR (400 MHz, DMSO-d₆) δ ppm 0.48-0.59 (m, 2H), 0.84-0.91 (m, 2H), 0.95-1.11 (m, 2H), 1.13-1.51 (m, 6H), 1.72-1.91 (m, 4H), 1.93-2.05 (m, 2H), 2.08-2.21 (m, 2H), 2.39-2.48 (m, 2H), 2.52-2.66 (m, 4H), 2.99-3.21 (m, 4H), 3.64-3.77 (m, 2H), 6.91 (br d, J = 7.3 Hz, 2H), 7.07 (br d, J = 8.1 Hz, 2H), 8.14 (s, 1H), 8.26 (br d, J = 7.8 Hz, 1H), 8.44 (br s, 1H), 8.61 (s, 1H), 8.96-9.03 (m, 2H), 10.10 (d, J = 2.2 Hz, 1H), 10.77 (s, 1H). |
| P-94 | A10 | C40 | 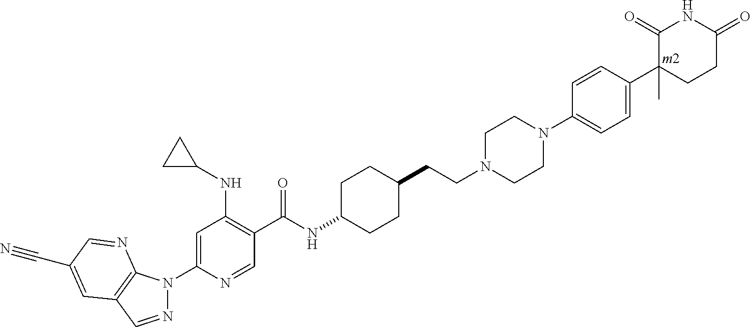 | 33 % yield as a white solid. LCMS method 2: retention time: 2.388 min, 99.9% purity at 215 nm with 99.2% ee, [M + 2H]²⁺ = 358.2, [M + H]⁺ = 715.4. ¹H NMR (400 MHz, DMSO-d₆) δ ppm 0.52-0.60 (m, 2H), 0.81-0.89 (m, 2H), 0.95-1.10 (m, 2H), 1.21-1.44 (m, 8H), 1.74-1.92 (m, 4H), 1.98-2.12 (m, 2H), 2.27-2.38 (m, 3H), 2.39-2.45 (m, 1H), 2.54-2.61 (m, 1H), 3.04-3.17 (m, 4H), 3.24-3.39 (m, 4H), 3.65-3.78 (m, 1H), 6.91 (d, J = 8.8 Hz, 2H), 7.10 (d, J = 8.6 Hz, 2H), 7.67 (s, 1H), 8.37 (d, J = 7.8 Hz, 1H), 8.55 (s, 1H), 8.59 (s, 1H), 8.65 (s, 1H), 9.01-9.04 (m, 1H), 9.04-9.07 (m, 1H), 10.84 (s, 1H). |
| P-95 | A10 | C41 | 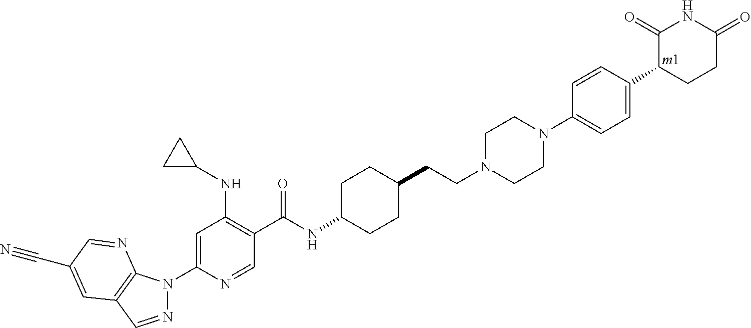 | 67 % yield as a white solid. LCMS method 2: retention time: 2.334 min, 99.5% purity at 215 nm with 75.0% ee, [M + 2H]²⁺ = 351.3, [M + H]⁺ = 701.4. ¹H NMR (400 MHz, DMSO-d₆) δ ppm 0.52-0.59 (m, 2H), 0.82-0.88 (m, 2H), 0.96-1.09 (m, 2H), 1.22-1.45 (m, 6H), 1.76-1.91 (m, 4H), 1.95-2.05 (m, 1H), 2.07-2.19 (m, 1H), 2.30-2.38 (m, 2H), 2.41-2.48 (m, 3H), 2.53-2.68 (m, 3H), 3.07-3.14 (m, 4H), 3.72 (dd, J = 11.0, 4.9 Hz, 2H), 6.88 (d, J = 8.6 Hz, 2H), 7.04 (d, J = 8.6 Hz, 2H), 7.67 (s, 1H), 8.37 (d, J = 7.8 Hz, 1H), 8.55 (s, 1H), 8.59 (s, 1H), 8.65 (s, 1H), 9.02 (d, J = 1.7 Hz, 1H), 9.05 (d, J = 1.7 Hz, 1H), 10.76 (s, 1H). |
| P-96 | A10 | C41 | 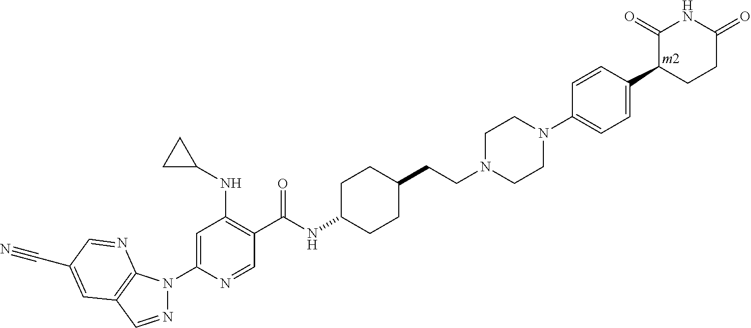 | 67 % yield as a white solid. LCMS method 2: retention time: 2.320 min, 99.9 % purity at 215 nm with 59.5% ee, [M + 2H]²⁺ = 351.3, [M + H]⁺ = 701.3. ¹H NMR (400 MHz, DMSO-d₆) δ ppm 0.53-0.59 (m, 2H), 0.81-0.89 (m, 2H), 0.95-1.08 (m, 2H), 1.21-1.43 (m, 6H), 1.74-1.91 (m, 4H), 1.97-2.04 (m, 1H), 2.07-2.19 (m, 2H), 2.31-2.39 (m, 2H), 2.42-2.45 (m, 1H), 2.54-2.68 (m, 3H), 3.09-3.13 (m, 4H), 3.29 (m, 1H), 3.68-3.76 (m, 2H), 6.89 (d, J = 8.8 Hz, 2H), 7.05 (d, J = 8.8 Hz, 2H), 7.67 (s, 1H), 8.38 (d, J = 7.8 Hz, 1H), 8.55 (s, 1H), 8.59 (s, 1H), 8.65 (s, 1H), 9.02 (d, J = 2.0 Hz, 1H), 9.06 (d, J = 2.0 Hz, 1H), 10.77 (s, 1H). |
| P-97 | A32 | C12 | 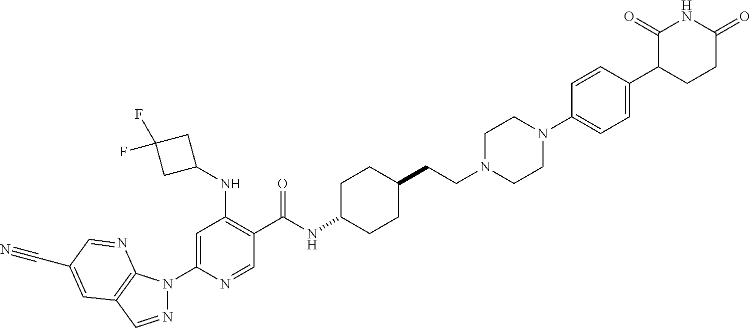 | 19 % yield as a white solid. LCMS method 2: retention time: 2.459 min, 98.1 % purity at 215 nm, [M + 2H]²⁺ = 376.2, [M + H]⁺ = 751.4. ¹H NMR (400 MHz, DMSO-d₆) δ ppm 0.98-1.09 (m, 2H), 1.21-1.44 (m, 6H), 1.77-1.84 (m, 2H), 1.86-1.94 (m, 2H), 1.97-2.05 (m, 1H), 2.08-2.21 (m, 2H), 2.31-2.39 (m, 2H), 2.42-2.49 (m, 3H), 2.58-2.73 (m, 3H), 3.08-3.14 (m, 4H), 3.14-3.23 (m, 2H), 3.68-3.79 (m, 2H), 3.96-4.04 (m, 1H), 6.89 (d, J = 8.8 Hz, 2H), 7.05 (d, J = 8.6 Hz, 2H), 7.27 (s, 1H),8.44 (d, J = 7.8 Hz, 1H), 8.64 (d, J = 5.4 Hz, 2H), 8.75 (d, J = 5.1 Hz, 1H), 9.02 (d, J = 2.2 Hz, 1H), 9.08 (d, J = 2.0 Hz, 1H), 10.77 (s, 1H). ¹⁹NMR (377 MHz, DMSO-d6) δ ppm −94.62-−93.00 (m, 1F), −83.26-−81.69 (m, 1F). |
| P-98 | A10 | C42 | 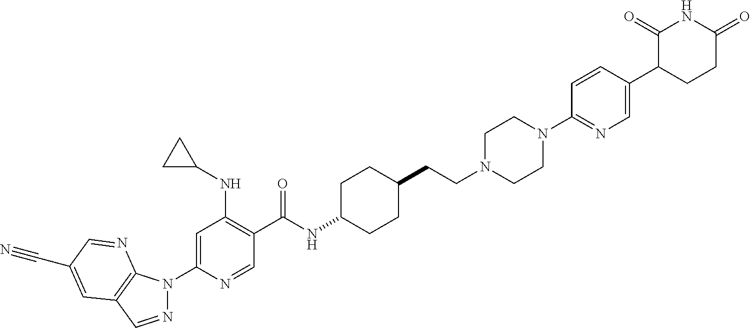 | 29 % yield as a white solid. LCMS method 2: retention time: 2.213 min, 96.2 % purity at 215 nm, [M + 2H]²⁺ = 351.8, [M + H]⁺ = 702.4. ¹H NMR (400 MHz, DMSO-d₆) δ ppm 0.52-0.60 (m, 2H), 0.81-0.89 (m, 2H), 0.96-1.09 (m, 2H), 1.19-1.45 (m, 5H), 1.74-1.91 (m, 4H), 1.92-2.02 (m, 1H), 2.10-2.25 (m, 1H), 2.30-2.39 (m, 2H), 2.41-2.47 (m, 4H), 2.55-2.61 (m, 1H), 2.62-2.73 (m, 1H), 3.42-3.48 (m, 4H), 3.65-3.80 (m, 2H), 6.79 (d, J = 9.0 Hz, 1H), 7.39 (dd, J = 8.7, 2.3 Hz, 1H), 7.67 (s, 1H), 7.95 (d, J = 2.2 Hz, 1H), 8.38 (d, J = 7.6 Hz, 1H), 8.55 (s, 1H), 8.59 (s, 1H), 8.66 (s, 1H), 9.02 (s, 1H), 9.06 (s, 1H), 10.80 (s, 1H). One proton was not apparent by ¹H NMR. |
| P-99 | A33 | C12 | 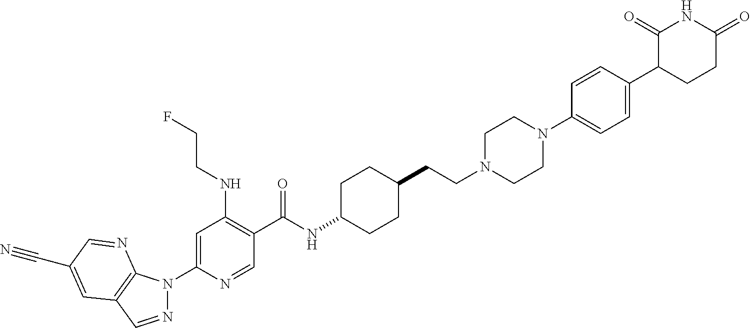 | 28 % yield as a tan solid. LCMS method 2: retention time: 2.207 min, 98.3 % purity at 215 nm, [M + 2H]²⁺ = 354.3, [M + H]⁺ = 707.4. ¹H NMR (400 MHz, DMSO-d₆) δ ppm 0.97-1.17 (m, 3H), 1.23-1.43 (m, 6H), 1.78-1.84 (m, 2H), 1.85-1.93 (m, 2H), 1.97-2.05 (m, 1H), 2.08-2.17 (m, 1H), 2.30-2.36 (m, 2H), 2.41-2.48 (m, 2H), 2.56-2.64 (m, 2H), 3.07-3.14 (m, 4H), 3.53-3.59 (m, 1H), 3.60-3.65 (m, 1H), 3.69-3.76 (m, 2H), 4.59 (t, J = 4.8 Hz, 1H), 4.69-4.74 (m, 1H), 6.89 (d, J = 8.6 Hz, 2H), 7.05 (d, J = 8.6 Hz, 2H), 7.32 (s, 1H), 8.38-8.44 (m, 1H), 8.60 (s, 1H), 8.64 (s, 1H), 8.66-8.74 (m, 1H), 9.00-9.05 (m, 2H), 10.77 (s, 1H). |
| P-100 | A34 | C12 | 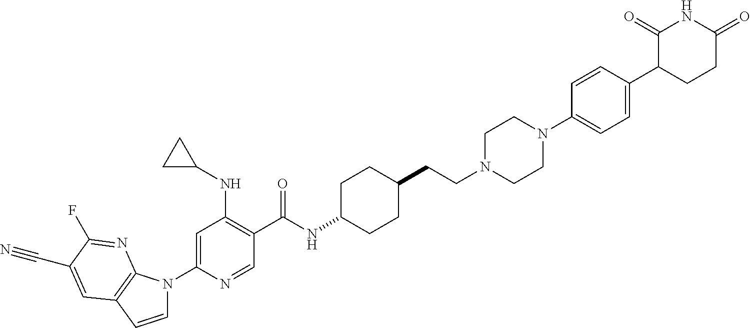 | 23 % yield as a tan solid. LCMS method 2: retention time: 2.774 min, 98.1 % purity at 215 nm, [M + 2H]²⁺ = 359.7, [M + H]⁺ = 718.4. ¹H NMR (400 MHz, DMSO-d₆) δ ppm 0.58-0.64 (m, 2H), 0.86-0.94 (m, 2H), 0.96-1.07 (m, 2H), 1.22-1.45 (m, 6H), 1.75-1.89 (m, 4H), 1.96-2.04 (m, 1H), 2.08-2.19 (m, 1H), 2.30-2.38 (m, 2H), 2.42-2.48 (m, 3H), 2.56-2.68 (m, 2H), 3.07-3.14 (m, 4H), 3.67-3.76 (m, 2H), 6.88 (d, J = 8.6 Hz, 2H), 6.93 (d, J = 3.9 Hz, 1H), 7.04 (d, J = 8.6 Hz, 2H), 8.27 (s, 1H), 8.33 (d, J = 7.8 Hz, 1H), 8.49 (d, J = 3.9 Hz, 1H), 8.56 (s, 1H), 8.64 (s, 1H), 8.86 (d, J = 8.8 Hz, 1H), 10.77 (s, 1H). ¹⁹NMR (377 MHz, DMSO-d6) δ ppm −69.75 (d, J = 8.2 Hz, 1F). One proton was not apparent by ¹H NMR. |
| P-101 | A35 | C12 | 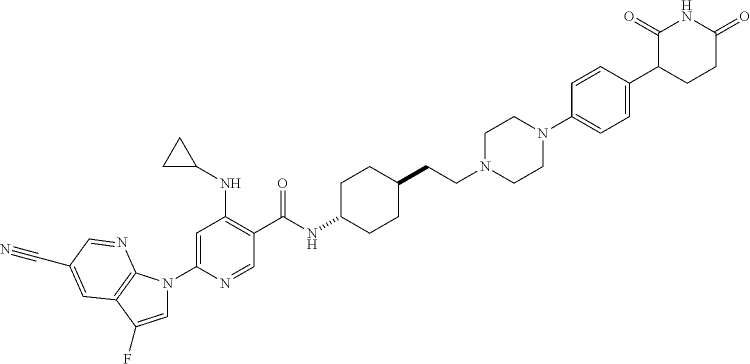 | 47 % yield as a light pink solid. LCMS method 2: retention time: 2.774 min, 99.5 % purity at 215 nm, [M + 2H]²⁺ = 359.8, [M + H]⁺ = 718.4. ¹H NMR (400 MHz, DMSO-d₆) δ ppm 0.55-0.62 (m, 2H), 0.89-0.95 (m, 2H), 0.97-1.08 (m, 2H), 1.12-1.44 (m, 6H), 1.75-1.90 (m, 4H), 1.96-2.05 (m, 1H), 2.06-2.21 (m, 1H), 2.36 (br t, J = 7.5 Hz, 2H), 2.42-2.50 (m, 3H), 2.53-2.72 (m, 3H), 3.08-3.14 (m, 4H), 3.66-3.75 (m, 2H), 6.89 (d, J = 8.8 Hz, 2H), 7.05 (d, J = 8.6 Hz, 2H), 8.32 (d, J = 7.6 Hz, 1H), 8.50-8.56 (m, 3H), 8.63 (s, 1H), 8.84 (s, 1H), 8.91 (d, J = 1.7 Hz, 1H), 10.77 (s, 1H). ¹⁹NMR (377 MHz, DMSO-d6) δ ppm −169.72 (s, 1F). |
| P-105 | A36 | C12 | 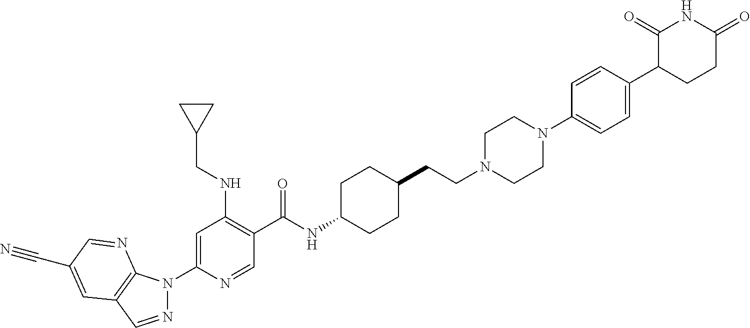 [SMILES: N#Cc1cnc2c(cnn2-c2cc(NCC3CC3)c(C(=O)N[C@H]3CC[C@H](CCN4CCN(c5ccc(C6CCC(=O)NC6=O)cc5)CC4)CC3)cn2)c1] | 40 % yield as a white solid. LCMS method 2: retention time: 2.357 min, 99.9 % purity at 215 nm, [M + 2H]²⁺ = 358.2, [M + H]⁺ = 715.4. ¹H NMR (400 MHz, DMSO-d₆) δ ppm 0.27-0.33 (m, 2H), 0.51-0.57 (m, 2H), 0.95-1.19 (m, 4H), 1.21-1.49 (m, 6H), 1.77-1.91 (m, 4H), 1.96-2.05 (m, 1H), 2.07-2.18 (m, 1H), 2.33-2.39 (m, 2H), 2.41-2.47 (m, 1H), 2.58-2.68 (m, 1H), 3.07-3.14 (m, 6H), 3.69-3.79 (m, 2H), 6.88 (d, J = 8.6 Hz, 2H), 7.05 (d, J = 8.3 Hz, 2H), 7.27 (s, 1H), 8.37 (d, J = 7.6 Hz, 1H), 8.56-8.66 (m, 3H), 8.96-9.09 (m, 2H), 10.77 (s, 1H). Two protons were not apparent by ¹H NMR. |
| P-106 | A37 | C12 | 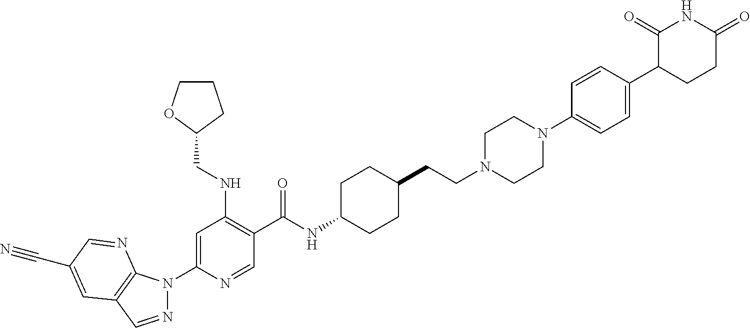 [SMILES: N#Cc1cnc2c(cnn2-c2cc(NC[C@H]3CCCO3)c(C(=O)N[C@H]3CC[C@H](CCN4CCN(c5ccc(C6CCC(=O)NC6=O)cc5)CC4)CC3)cn2)c1] | 55 % yield as a light yellow solid. LCMS method 2: retention time: 2.247 min, 98.6 % purity at 215 nm, [M + 2H]²⁺ = 373.2, [M + H]⁺ = 745.4. ¹H NMR (400 MHz, DMSO-d₆) δ ppm 0.97-1.16 (s, 3H), 1.23-1.45 (m, 5H), 1.55-1.66 (m, 1H), 1.77-1.93 (m, 6H), 1.94-2.05 (m, 2H), 2.07-2.19 (m, 1H), 2.30-2.39 (m, 2H), 2.42-2.49 (m, 2H), 2.57-2.69 (m, 2H), 3.09-3.15 (m, 4H), 3.18-3.26 (m, 1H), 3.35-3.42 (m, 2H), 3.65-3.85 (m, 4H), 4.03-4.11 (m, 1H), 6.89 (d, J = 8.8 Hz, 2H), 7.05 (d, J = 8.8 Hz, 2H), 7.31 (s, 1H), 8.37 (d, J = 7.8 Hz, 1H), 8.58 (s, 1H), 8.62-8.67 (m, 2H), 9.02 (d, J = 2.0 Hz, 1H), 9.05 (d, J = 2.0 Hz, 1H), 10.77 (s, 1H). |
| P-107 | A38 | C12 | 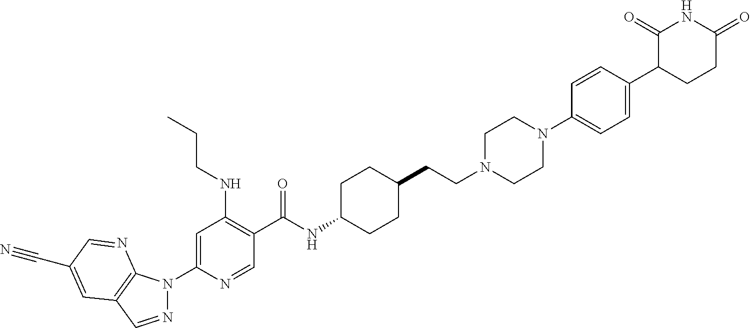 [SMILES: CCCNc1cc(-n2ncc3cc(C#N)cnc32)ncc1C(=O)N[C@H]1CC[C@H](CCN2CCN(c3ccc(C4CCC(=O)NC4=O)cc3)CC2)CC1] | 37 % yield as an off-white solid. LCMS method 2: retention time: 2.332 min, 99.9 % purity at 215 nm, [M + 2H]²⁺ = 352.3, [M + H]⁺ = 703.4. ¹H NMR (400 MHz, DMSO-d₆) δ ppm 0.97 (t, J = 7.5 Hz, 3H), 0.99-1.16 (m, 3H), 1.20-1.48 (m, 6H), 1.64 (dq, J = 14.4, 7.3 Hz, 2H), 1.75-1.83 (m, 2H), 1.84-1.92 (m, 2H), 1.97-2.04 (m, 1H), 2.07-2.18 (m, 1H), 2.32-2.38 (m, 2H), 2.41-2.47 (m, 1H), 2.58-2.67 (m, 1H), 3.07-3.14 (m, 4H), 3.16-3.24 (m, 3H), 3.69-3.78 (m, 2H), 6.88 (d, J = 8.6 Hz, 2H), 7.04 (d, J = 8.6 Hz, 2H), 7.28 (s, 1H), 8.36 (d, J = 7.6 Hz, 1H), 8.51-8.60 (m, 2H), 8.63 (s, 1H), 8.97-9.07 (m, 2H), 10.77 (s, 1H). |
| P-108 | A39 | C12 | 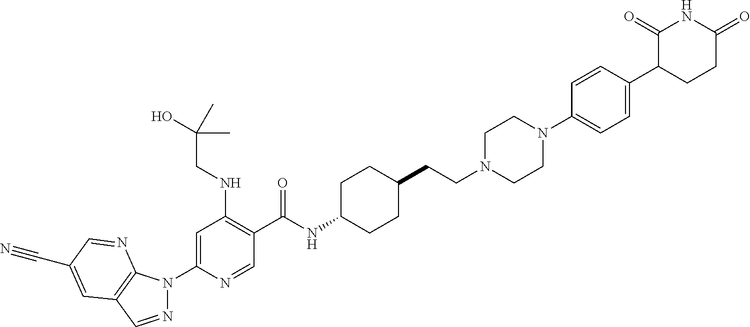 [SMILES: CC(C)(O)CNc1cc(-n2ncc3cc(C#N)cnc32)ncc1C(=O)N[C@H]1CC[C@H](CCN2CCN(c3ccc(C4CCC(=O)NC4=O)cc3)CC2)CC1] | 7.5 % yield as a white solid. LCMS method 2: retention time: 2.114 min, 97.3 % purity at 215 nm, [M + 2H]²⁺ = 367.3, [M + H]⁺ = 733.4. ¹H NMR (400 MHz, DMSO-d₆) δ ppm 0.96-1.11 (m, 2H), 1.12-1.20 (m, 6H), 1.20-1.51 (m, 6H), 1.72-1.94 (m, 4H), 1.94-2.07 (m, 1H), 2.10-2.21 (m 1H), 2.34-2.47 (m , 3H), 2.53-2.65 (m, 3H), 3.02-3.23 (m , 6H), 3.68-3.83 (m, 2H), 4.69 (s, 1H), 6.89 (d, J = 8.8 Hz, 2H), 7.05 (d, J = 8.6 Hz, 2H), 7.25 (s, 1H), 8.34 (d, J = 7.6 Hz, 1H), 8.57 (s, 1H), 8.63 (s, 1H), 8.70-8.78 (m, 1H), 9.01 (d, J = 2.0 Hz, 1H), 9.04 (d, J = 2.0 Hz, 1H), 10.77 (s, 1H). One proton was not apparent by ¹H NMR. |
| P-109 | A40 | C12 | 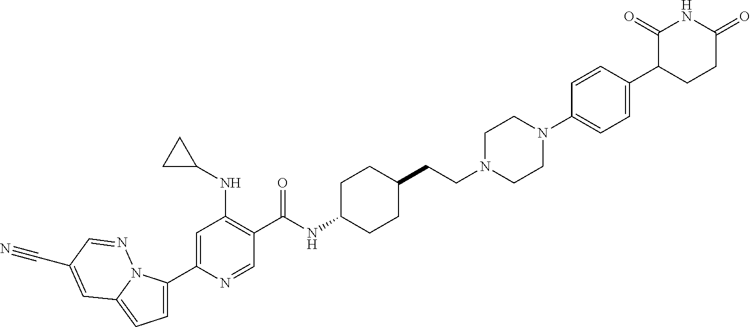 [SMILES: N#Cc1cnn2c(-c3cc(NC4CC4)c(C(=O)N[C@H]4CC[C@H](CCN5CCN(c6ccc(C7CCC(=O)NC7=O)cc6)CC5)CC4)cn3)ccc2c1] | 30 % yield as a yellow solid. LCMS method 6: retention time: 3.353 min, 98.8 % purity at 215 nm, [M + 2H]²⁺ = 350.8, [M + H]⁺ = 700.4. ¹H NMR (400 MHz, DMSO-d₆) δ ppm 0.52-0.59 (m, 2H), 0.93 (m, 2H), 0.98-1.09 (m, 2H), 1.20-1.43 (m, 6H), 1.74-1.90 (m, 4H), 1.95-2.05 (m, 1H), 2.07-2.19 (m, 1H), 2.29-2.39 (m, 2H), 2.41-2.46 (m, 1H), 2.52-2.69 (m, 3H), 3.05-3.15 (m, 4H), 3.65-3.77 (m, 2H), 6.88 (d, J = 8.8 Hz, 2H), 7.04 (d, J = 8.6 Hz, 2H), 7.11 (d, J = 4.6 Hz, 1H), 7.85 (d, J = 4.9 Hz, 1H), 8.27 (d, J = 7.8 Hz, 1H), 8.50 (s, 1H), 8.53 (s, 1H), 8.67 (s, 1H), 8.74 (d, J = 2.0 Hz, 1H), 8.84 (d, J = 2.0 Hz, 1H), 10.77 (s, 1H). Two protons were not apparent by ¹H NMR. |
| P-112 | A41 | C12 | 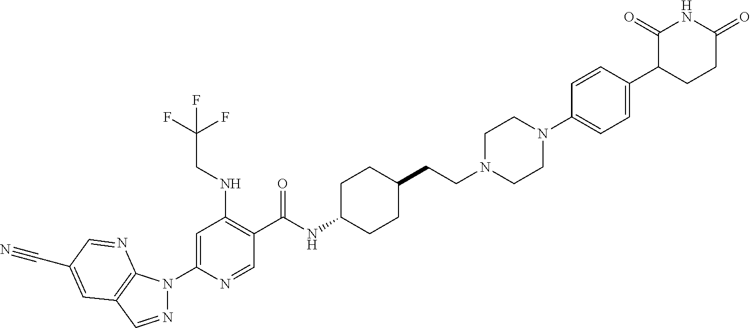 [SMILES: N#Cc1cnc2c(cnn2-c2cc(NCC(F)(F)F)c(C(=O)N[C@H]3CC[C@H](CCN4CCN(c5ccc(C6CCC(=O)NC6=O)cc5)CC4)CC3)cn2)c1] | 14 % yield as an off-white solid. LCMS method 2: retention time: 2.435 min, 99.9% purity at 215 nm, [M + H]⁺ = 743.3. ¹H NMR (400 MHz, DMSO-d₆) δ ppm 1.00-1.09 (m, 2H), 1.23-1.45 (m, 7H), 1.78-1.85 (m, 2H), 1.87-1.93 (m, 2H), 1.97-2.04 (m, 1H), 2.08-2.18 (m, 1H), 2.36-2.45 (m, 3H), 2.54 (br d, J = 0.7 Hz, 2H), 2.59-2.68 (m, 1H), 3.10-3.16 (m, 4H), 3.70-3.77 (m, 2H), 4.25-4.34 (m, 2H), 6.89 (br d, J = 8.8 Hz, 2H), 7.05 (d, J = 8.6 Hz, 2H), 7.51 (s, 1H), 8.51 (br d, J = 7.6 Hz, 1H), 8.65 (d, J = 7.3 Hz, 2H), 8.85 (br t, J = 6.8 Hz, 1H), 9.02-9.05 (m, 2H), 10.77 (s, 1H). ¹⁹NMR (377 MHz, DMSO-d6) δ ppm −70.20 (t, J = 9.5 Hz, 3F). |
| P-113 | A42 | C12 | 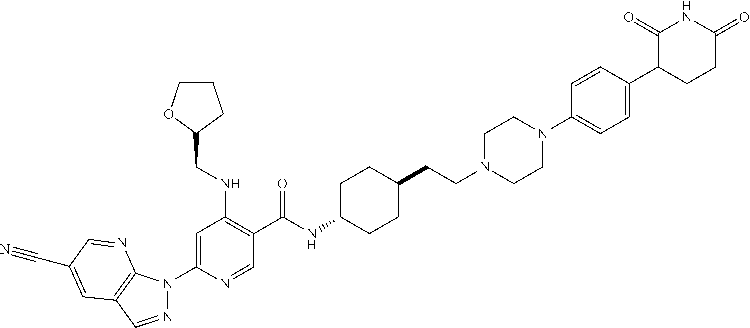 | 14 % yield as an off-white solid. LCMS method 2: retention time: 2.231 min, 95.2 % purity at 215 nm, [M + H]⁺ = 745.4. ¹H NMR (400 MHz, DMSO-d₆) δ ppm 0.98-1.10 (m, 2H), 1.22-1.46 (m, 7H), 1.55-1.65 (m, 1H), 1.76-1.93 (m, 7H), 1.97-2.05 (m, 2H), 2.07-2.19 (m, 1H), 2.32-2.44 (m, 3H), 2.58-2.70 (m, 2H), 3.19-3.26 (m, 1H), 3.29-3.42 (m, 4H), 3.63-3.82 (m, 5H), 4.03-4.10 (m, 1H), 6.89 (br d, J = 8.6 Hz, 2H), 7.05 (br d, J = 8.6 Hz, 2H), 7.31 (s, 1H), 8.37 (br d, J = 8.3 Hz, 1H), 8.57 (s, 1H), 8.62-8.68 (m, 2H), 9.01 (d, J = 2.0 Hz, 1H), 9.04 (d, J = 1.7 Hz, 1H), 10.78 (s, 1H). |
| P-114 | A43 | C12 | 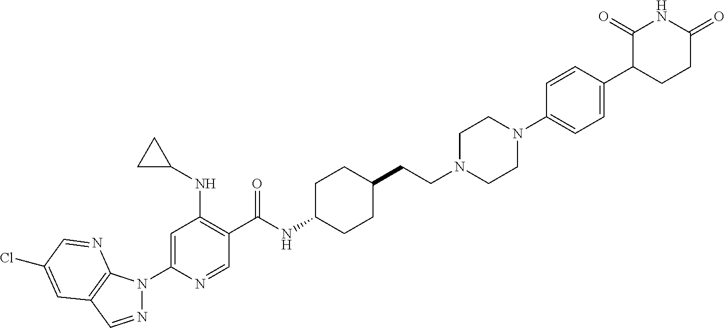 [SMILES: O=C1CCC(c2ccc(N3CCN(CC[C@H]4CC[C@H](NC(=O)c5cnc(-n6ncc7cc(Cl)cnc76)cc5NC5CC5)CC4)CC3)cc2)C(=O)N1] | 26 % yield as an off-white solid. LCMS method 2: retention time: 2.401 min, 99.0 % purity at 215 nm, [M + 2H]²⁺ = 355.7, [M + H]⁺ = 710.4. ¹H NMR (400 MHz, DMSO-d₆) δ ppm 0.52-0.60 (m, 2H), 0.82-0.90 (m, 2H), 0.96-1.09 (m, 2H), 1.19-1.46 (m, 6H), 1.76-1.91 (m, 4H), 1.96-2.05 (m, 1H), 2.07-2.19 (m, 1H), 2.38-2.45 (m, 2H), 2.53-2.68 (m, 6H), 3.09-3.18 (m, 4H), 3.68-3.77 (m, 2H), 6.89 (br d, J = 8.6 Hz, 2H), 7.05 (d, J = 8.6 Hz, 2H), 7.71 (s, 1H), 8.35 (br d, J = 7.8 Hz, 1H), 8.46 (s, 1H), 8.55 (d, J = 2.4 Hz, 2H), 8.58 (s, 1H), 8.71 (d, J = 2.4 Hz, 1H), 10.77 (s, 1H). |
| P-115 | A44 | C12 | 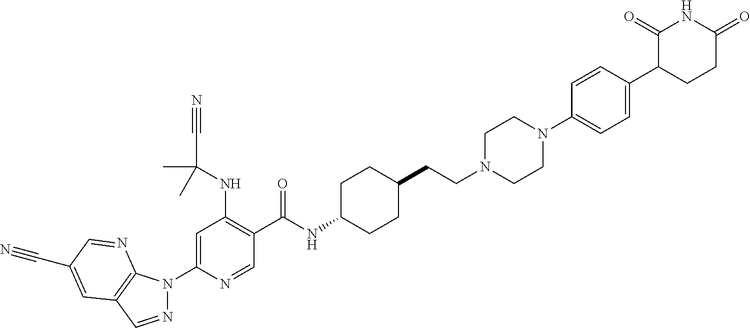 [SMILES: CC(C)(C#N)Nc1cc(-n2ncc3cc(C#N)cnc32)ncc1C(=O)N[C@H]1CC[C@H](CCN2CCN(c3ccc(C4CCC(=O)NC4=O)cc3)CC2)CC1] | 28 % yield as an off-white solid. LCMS method 2: retention time: 2.396 min, 99.0 % purity at 215 nm, [M + 2H]²⁺ = 364.7, [M + H]⁺ = 728.4. 1H NMR (400 MHz, DMSO-d₆) δ ppm 0.97-1.16 (m, 3H), 1.22-1.44 (m, 5H), 1.83 (s, 10H), 1.96-2.05 (m, 1H), 2.08-2.19 (m, 1H), 2.31-2.39 (m, 2H), 2.42-2.47 (m, 1H), 2.55-2.69 (m, 3H), 3.07-3.14 (m, 4H), 3.69-3.81 (m, 3H), 6.89 (d, J = 8.6 Hz, 2H), 7.05 (d, J = 8.6 Hz, 2H), 7.78 (s, 1H), 8.19 (s, 1H), 8.56 (d, J = 7.6 Hz, 1H), 8.70 (s, 1H), 8.76 (s, 1H), 9.05 (d, J = 1.7 Hz, 1H), 9.06 (d, J = 1.7 Hz, 1H), 9.18 (s, 1H), 10.77 (s, 1H). |
| P-116 | A10 | C31 | 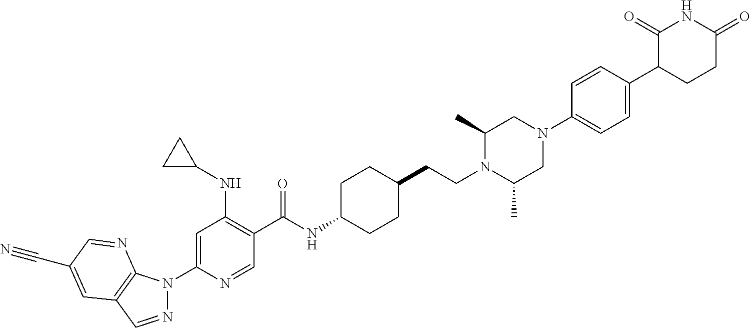 [SMILES: C[C@H]1CN(c2ccc(C3CCC(=O)NC3=O)cc2)C[C@H](C)N1CC[C@H]1CC[C@H](NC(=O)c2cnc(-n3ncc4cc(C#N)cnc43)cc2NC2CC2)CC1] | 34 % yield as a white solid. LCMS method 2: retention time: 2.427 min, 98.9 % purity at 215 nm, [M + 2H]²⁺ = 365.3, [M + H]⁺ = 729.4. ¹H NMR (400 MHz, DMSO-d₆) δ ppm 0.53-0.61 (m, 2H), 0.81-0.91 (m, 2H), 0.98-1.09 (m, 8H), 1.25-1.44 (m, 5H), 1.70-1.92 (m, 4H), 1.95-2.04 (m, 1H), 2.07-2.19 (m, 1H), 2.21-2.36 (m, 1H), 2.39-2.48 (m, 1H), 2.55-2.77 (m, 3H), 2.81-2.91 (m, 2H), 2.94-3.02 (m, 2H), 3.07-3.21 (m, 2H), 3.72 (br dd, J = 11.1, 5.0 Hz, 2H), 6.87 (br d, J = 8.6 Hz, 2H), 7.04 (d, J = 8.6 Hz, 2H), 7.67 (s, 1H), 8.38 (br d, J = 7.8 Hz, 1H), 8.56 (s, 1H), 8.60 (s, 1H), 8.66 (s, 1H), 9.02 (d, J = 2.0 Hz, 1H), 9.06 (d, J = 2.0 Hz, 1H), 10.77 (s, 1H). |
| P-117 | A45 | C12 | 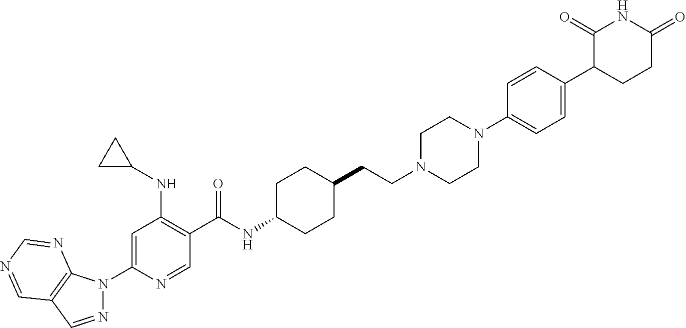 [SMILES: O=C1CCC(c2ccc(N3CCN(CC[C@H]4CC[C@H](NC(=O)c5cnc(-n6ncc7cncnc76)cc5NC5CC5)CC4)CC3)cc2)C(=O)N1] | 43 % yield as a white solid. LCMS method 2: retention time: 2.177 min, 99.9 % purity at 215 nm, [M + 2H]²⁺ = 339.2, [M + H]⁺ = 677.4. ¹H NMR (400 MHz, DMSO-d₆) δ ppm 0.54-0.60 (m, 2H), 0.83-0.89 (m, 2H), 0.97-1.15 (m, 2H), 1.19-1.46 (m, 5H), 1.75-1.91 (m, 4H), 1.97-2.04 (m, 1H), 2.07-2.19 (m, 1H), 2.31-2.39 (m, 2H), 2.42-2.49 (m, 3H), 2.55-2.70 (m, 2H), 3.08-3.15 (m, 4H), 3.73 (dd, J = 11.2, 4.9 Hz, 2H), 6.89 (d, J = 8.8 Hz, 2H), 7.05 (d, J = 8.6 Hz, 2H), 7.72 (s, 1H), 8.38 (d, J = 7.8 Hz, 1H), 8.57 (s, 1H), 8.60 (s, 1H), 8.68 (s, 1H), 9.17 (s, 1H), 9.48 (s, 1H), 10.77 (s, 1H). Two protons were not apparent by ¹H NMR. |
| P-118 | A46 | C12 | 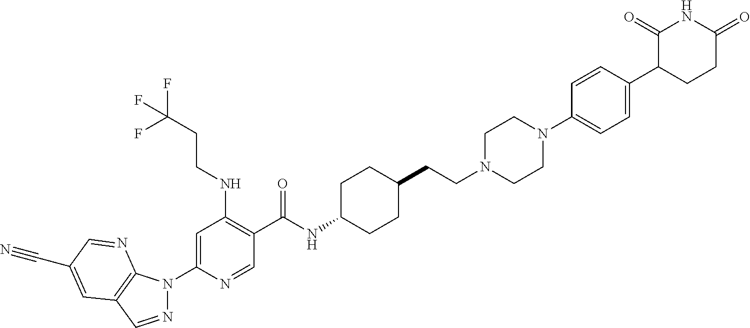 [SMILES: N#Cc1cnc2c(cnn2-c2cc(NCCC(F)(F)F)c(C(=O)N[C@H]3CC[C@H](CCN4CCN(c5ccc(C6CCC(=O)NC6=O)cc5)CC4)CC3)cn2)c1] | 39 % yield as an off-white solid. LCMS method 2: retention time: 2.435 min, 99.9 % purity at 215 nm, [M + 2H]²⁺ = 379.2, [M + H]⁺ = 757.3. ¹H NMR (400 MHz, DMSO-d₆) δ ppm 0.97-1.15 (m, 2H), 1.20-1.46 (m, 6H), 1.74-1.93 (m, 4H), 1.95-2.06 (m, 1H), 2.06-2.20 (m, 1H), 2.30-2.40 (m, 2H), 2.41-2.47 (m, 1H), 2.55-2.79 (m, 4H), 3.05-3.16 (m, 4H), 3.32 (s, 2H), 3.50-3.59 (m, 2H), 3.67-3.80 (m, 2H), 6.88 (d, J = 8.6 Hz, 2H), 7.05 (d, J = 8.6 Hz, 2H), 7.35 (s, 1H), 8.39 (d, J = 7.8 Hz, 1H), 8.60 (s, 1H), 8.61-8.68 (m, 2H), 8.98-9.07 (m, 2H), 10.77 (s, 1H). ¹⁹F NMR (377 MHz, DMSO-d₆) δ ppm −63.49 (s, 3F). |
| P-119 | A47 | C12 | 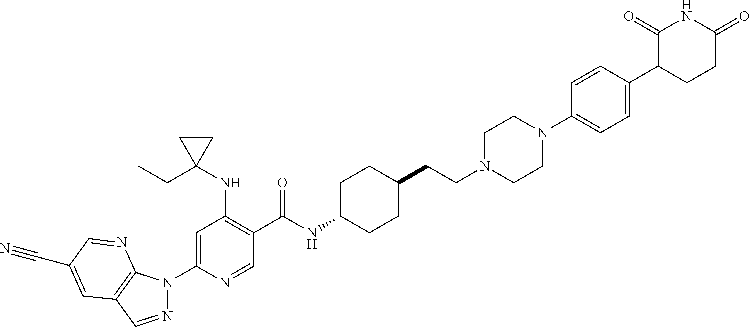 [SMILES: CCC1(Nc2cc(-n3ncc4cc(C#N)cnc43)ncc2C(=O)N[C@H]2CC[C@H](CCN3CCN(c4ccc(C5CCC(=O)NC5=O)cc4)CC3)CC2)CC1] | 25 % yield as an off-white solid. LCMS method 2: retention time: 2.501 min, 97.7 % purity at 215 nm, [M − HCOOH + 2H]⁺ = 365.3, [M + H]⁺ = 729.4. ¹H NMR (400 MHz, DMSO-d₆) δ ppm 0.73-0.82 (m, 4H), 0.93 (t, J = 7.2 Hz, 3H), 0.98-1.11 (m, 2H), 1.13-1.47 (m, 7H), 1.55-1.68 (m, 2H), 1.74-1.90 (m, 4H), 1.95-2.05 (m, 1H), 2.06-2.21 (m, 1H), 2.30-2.41 (m, 2H), 2.41-2.47 (m, 1H), 2.57-2.70 (m, 1H), 3.06-3.16 (m, 4H), 3.67-3.79 (m, 2H), 6.88 (d, J = 8.3 Hz, 2H), 7.05 (d, J = 8.3 Hz, 2H), 7.67 (s, 1H), 8.16 (s, 1H), 8.37 (d, J = 7.6 Hz, 1H), 8.60 (s, 1H), 8.65 (s, 1H), 8.80 (s, 1H), 9.02 (s, 1H), 9.05 (s, 1H), 10.77 (s, 1H). Three protons were not apparent by ¹H NMR. |
| P-120 | A10 | C43 | 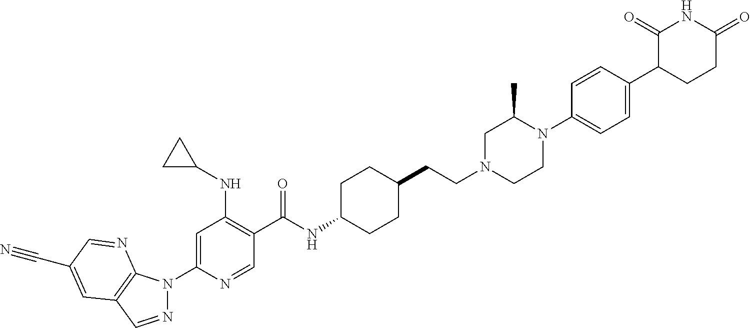 [SMILES: C[C@@H]1CN(CC[C@H]2CC[C@H](NC(=O)c3cnc(-n4ncc5cc(C#N)cnc54)cc3NC3CC3)CC2)CCN1c1ccc(C2CCC(=O)NC2=O)cc1] | 35 % yield as an white solid. LCMS method 2: retention time: 2.354 min, 99.9 % purity at 215 nm, [M + 2H]²⁺ = 358.3, [M + H]⁺ = 715.4. ¹H NMR (400 MHz, DMSO-d₆) δ ppm 0.53-0.61 (m, 2H), 0.82-0.90 (m, 2H), 0.97-1.13 (m, 5H), 1.29-1.44 (m, 5H), 1.76-1.91 (m, 4H), 2.00-2.18 (m, 3H), 2.22-2.37 (m, 3H), 2.40-2.48 (m, 1H), 2.58-2.71 (m, 3H), 2.81-2.89 (m, 1H), 2.90-2.99 (m, 1H), 3.21-3.29 (m, 1H), 3.68-3.77 (m, 2H), 3.90-3.99 (m, 1H), 6.84 (d, J = 8.8 Hz, 2H), 7.04 (d, J = 8.8 Hz, 2H), 7.67 (s, 1H), 8.38 (d, J = 7.8 Hz, 1H), 8.56-8.58 (m, 1H), 8.60 (s, 1H), 8.66 (s, 1H), 9.03 (d, J = 2.0 Hz, 1H), 9.06 (d, J = 2.0 Hz, 1H), 10.77 (s, 1H). |
| P-121 | A10 | C44 | 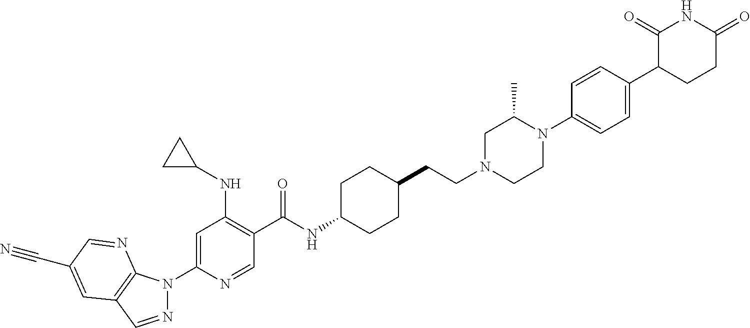 [SMILES: C[C@H]1CN(CC[C@H]2CC[C@H](NC(=O)c3cnc(-n4ncc5cc(C#N)cnc54)cc3NC3CC3)CC2)CCN1c1ccc(C2CCC(=O)NC2=O)cc1] | 86.6 % yield as an white solid. LCMS method 2: retention time: 2.360 min, 98.6 % purity at 215 nm, [M + 2H]²⁺ = 358.3, [M + H]⁺ = 715.4. ¹H NMR (400 MHz, DMSO-d₆) δ ppm 0.53-0.58 (m, 2H), 0.82-0.89 (m, 2H), 0.98-1.09 (m, 5H), 1.26-1.44 (m, 5H), 1.75-1.92 (m, 4H), 1.97-2.16 (m, 3H), 2.21-2.37 (m, 3H), 2.41-2.48 (m, 1H), 2.55-2.71 (m, 3H), 2.85 (br d, J = 10.0 Hz, 1H), 2.90-2.99 (m, 1H), 3.20-3.26 (m, 1H), 3.68-3.78 (m, 2H), 3.91-3.98 (m, 1H), 6.83 (d, J = 8.8 Hz, 2H), 7.04 (d, J = 8.8 Hz, 2H), 7.67 (s, 1H), 8.37 (d, J = 7.8 Hz, 1H), 8.56 (s, 1H), 8.59 (s, 1H), 8.65 (s, 1H), 9.02 (d, J = 2.0 Hz, 1H), 9.06 (d, J = 2.0 Hz, 1H), 10.77 (s, 1H). |
| P-122 | A48 | C12 | 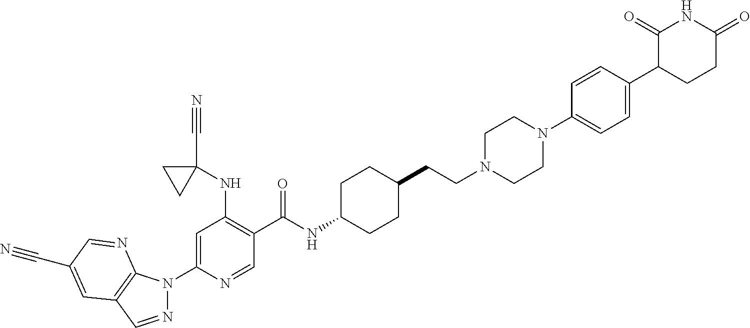 [SMILES: N#Cc1cnc2c(cnn2-c2cc(NC3(C#N)CC3)c(C(=O)N[C@H]3CC[C@H](CCN4CCN(c5ccc(C6CCC(=O)NC6=O)cc5)CC4)CC3)cn2)c1] | 26 % yield as a white solid. LCMS method 2: 99.9 % purity at 215 nm, [M + H]⁺ = 726.4, [M + 2H]²⁺ = 363.8. ¹H NMR (400 MHz, DMSO-d₆) δ ppm 0.96-1.09 (m, 2H), 1.22-1.46 (m, 8H), 1.68-1.75 (m, 2H), 1.76-1.84 (m, 2H), 1.85-1.94 (m, 2H), 1.94-2.06 (m, 1H), 2.06-2.19 (m, 2H), 2.31-2.39 (m, 2H), 2.41-2.46 (m, 1H), 2.57-2.69 (m, 2H), 3.07-3.14 (m, 4H), 3.68-3.79 (m, 3H), 6.89 (d, J = 8.6 Hz, 2H), 7.05 (d, J = 8.6 Hz, 2H), 7.85 (s, 1H), 8.51 (d, J = 7.8 Hz, 1H), 8.69 (s, 1H), 8.71 (s, 1H), 8.94 (s, 1H), 9.05 (d, J = 2.0 Hz, 1H), 9.09 (d, J = 2.0 Hz, 1H), 10.77 (s, 1H). |
| P-123 | A49 | C12 | 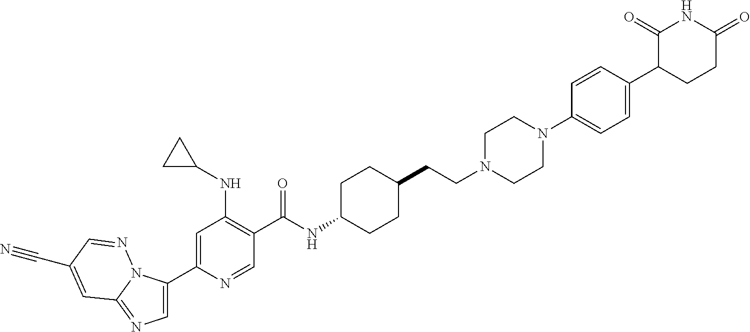 [SMILES: N#Cc1cnn2c(-c3cc(NC4CC4)c(C(=O)N[C@H]4CC[C@H](CCN5CCN(c6ccc(C7CCC(=O)NC7=O)cc6)CC5)CC4)cn3)cnc2c1] | 52 % yield as a yellow solid. LCMS method 2: Retention time: 1.988 min, 99.9 % purity at 215 nm, [M + H]⁺ = 701.4, [M + 2H]²⁺ = 351.2. ¹H NMR (400 MHz, DMSO-d₆) δ ppm 0.55-0.60 (m, 2H), 0.90-0.96 (m, 2H), 0.99-1.08 (m, 2H), 1.23-1.42 (m, 6H), 1.75-1.90 (m, 4H), 1.96-2.06 (m, 1H), 2.08-2.19 (m, 1H), 2.35-2.38 (m, 1H), 2.42-2.48 (m, 4H), 2.55-2.65 (m, 3H), 3.08-3.15 (m, 4H), 3.69-3.76 (m, 2H), 6.89 (d, J = 8.8 Hz, 2H), 7.05 (d, J = 8.8 Hz, 2H), 8.32 (d, J = 7.3 Hz, 1H), 8.41 (s, 1H), 8.56 (s, 1H), 8.69 (s, 1H), 8.71 (s, 1H), 9.10 (d, J = 2.2 Hz, 1H), 9.13 (d, J = 2.2 Hz, 1H), 10.77 (s, 1H). |
| P-124 | A10 | C45 | 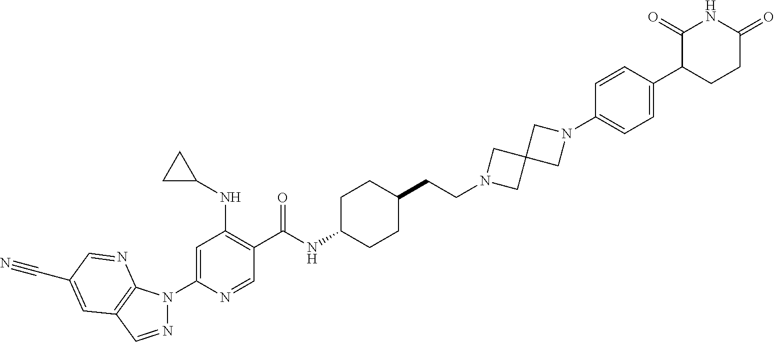 [SMILES: N#Cc1cnc2c(cnn2-c2cc(NC3CC3)c(C(=O)N[C@H]3CC[C@H](CCN4CC5(C4)CN(c4ccc(C6CCC(=O)NC6=O)cc4)C5)CC3)cn2)c1] | 17 % yield as a light yellow solid. LCMS method 2: Retention time: 2.335 min, 99.9 % purity at 215 nm, [M + H]⁺ = 713.4, [M + 2H]²⁺ = 357.3. ¹H NMR (400 MHz, DMSO-d₆) δ ppm 0.52-0.60 (m, 2H), 0.81-0.90 (m, 2H), 0.91-1.05 (m, 2H), 1.13-1.39 (m, 6H), 1.69-1.79 (m, 2H), 1.81-1.91 (m, 2H), 1.93-2.03 (m, 1H), 2.04-2.17 (m, 1H), 2.31-2.46 (m, 3H), 2.54-2.65 (m, 2H), 3.23 (s, 4H), 3.66-3.72 (m, 1H), 3.83 (s, 4H), 6.38 (d, J = 8.3 Hz, 2H), 6.99 (d, J = 8.3 Hz, 2H), 7.67 (s, 1H), 8.37 (d, J = 7.6 Hz, 1H), 8.54 (s, 1H), 8.59 (s, 1H), 8.65 (s, 1H), 9.00-9.04 (m, 1H), 9.04-9.08 (m, 1H), 10.74 (s, 1H). |
| P-125 | A10 | C46 | 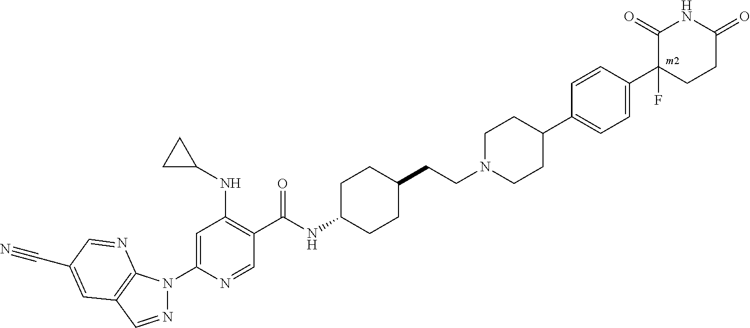 [SMILES: N#Cc1cnc2c(cnn2-c2cc(NC3CC3)c(C(=O)N[C@H]3CC[C@H](CCN4CCC(c5ccc(C6(F)CCC(=O)NC6=O)cc5)CC4)CC3)cn2)c1] | 49% yield as a white solid. LCMS method 2: Retention time: 2.400 min, 99.7 % purity at 215 nm, [M + H]⁺ = 718.3, [M + 2H]²⁺ = 359.8. ¹H NMR (400 MHz, DMSO-d₆) δ ppm 0.54-0.58 (m, 2H), 0.83-0.88 (m, 2H), 0.97-1.06 (m, 2H), 1.28-1.41 (m, 5H), 1.62-1.69 (m, 2H), 1.71-1.83 (m, 5H), 1.84-1.89 (m, 2H), 1.95-2.02 (m, 2H), 2.30-2.37 (m, 3H), 2.54-2.61 (m, 2H), 2.66-2.74 (m, 2H), 2.96-3.01 (m, 2H), 3.68-3.75 (m, 1H), 7.33-7.38 (m, 4H), 7.67 (s, 1H), 8.38 (d, J = 7.8 Hz, 1H), 8.54-8.56 (m, 1H), 8.60 (s, 1H), 8.66 (s, 1H), 9.02 (d, J = 2.2 Hz, 1H), 9.06 (d, J = 2.2 Hz, 1H), 11.37 (s, 1H). ¹⁹NMR (377 MHz, DMSO-d₆) δ ppm −146.45-−146.32 (m, 1F). |
| P-126 | A10 | C46 | 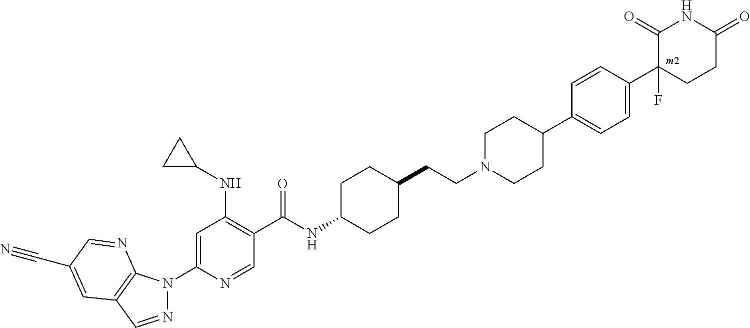 [SMILES: N#Cc1cnc2c(cnn2-c2cc(NC3CC3)c(C(=O)N[C@H]3CC[C@H](CCN4CCC(c5ccc(C6(F)CCC(=O)NC6=O)cc5)CC4)CC3)cn2)c1] | 40% yield as a white solid. LCMS method 2: Retention time: 2.418 min, 99.9 % purity at 215 nm, [M + H]⁺ = 718.4, [M + 2H]²⁺ = 359.8. ¹H NMR (400 MHz, DMSO-d₆) δ ppm 0.53-0.58 (m, 2H), 0.83-0.88 (m, 2H), 1.01-1.11 (m, 2H), 1.27-1.41 (m, 3H), 1.53-1.62 (m, 2H), 1.76-2.03 (m, 9H), 2.28-2.36 (m, 1H), 2.55-2.61 (m, 1H), 2.63-2.78 (m, 3H), 2.79-3.02 (m, 3H), 3.70-3.77 (m, 1H), 7.33-7.43 (m, 4H), 7.67 (s, 1H), 8.41 (d, J = 7.6 Hz, 1H), 8.54 (s, 1H), 8.60 (s, 1H), 8.66 (s, 1H), 9.03 (d, J = 2.0 Hz, 1H), 9.06 (d, J = 2.0 Hz, 1H), 11.39 (s, 1H). ¹⁹NMR (377 MHz, DMSO-d₆) δ ppm −147.18-−146.87 (m, 1F). Three protons were not apparent by ¹H NMR. |
| P-128 | A29 | C12 | 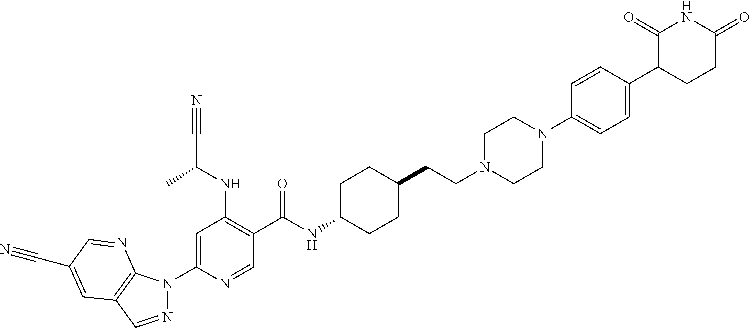 [SMILES: C[C@H](C#N)Nc1cc(-n2ncc3cc(C#N)cnc32)ncc1C(=O)N[C@H]1CC[C@H](CCN2CCN(c3ccc(C4CCC(=O)NC4=O)cc3)CC2)CC1] | 29% yield as a white solid. LCMS method 5: Retention time: 2.306 min, 99.9 % purity at 215 nm, [M + H]⁺ = 714.3, [M + 2H]²⁺ = 357.7. ¹H NMR (400 MHz, DMSO-d₆) δ ppm 0.98-1.11 (m, 2H), 1.20-1.46 (m, 6H), 1.67 (d, J = 6.8 Hz, 3H), 1.74-1.85 (m, 2H), 1.86-1.95 (m, 2H), 1.96-2.05 (m, 1H), 2.06-2.20 (m, 1H), 2.34-2.39 (m, 2H), 2.40-2.47 (m, 2H), 2.57-2.65 (m, 1H), 3.07-3.15 (m, 4H), 3.68-3.74 (m, 2H), 4.90-4.98 (m, 1H), 6.89 (d, J = 8.8 Hz, 2H), 7.05 (d, J = 8.6 Hz, 2H), 7.48 (s, 1H), 8.54 (d, J = 7.6 Hz, 1H), 8.69 (s, 1H), 8.71 (s, 1H), 8.77 (d, J = 7.1 Hz, 1H), 9.03-9.04 (m, 1H), 9.04-9.05 (m, 1H), 10.77 (s, 1H). |
| P-131 | A50 | C12 | 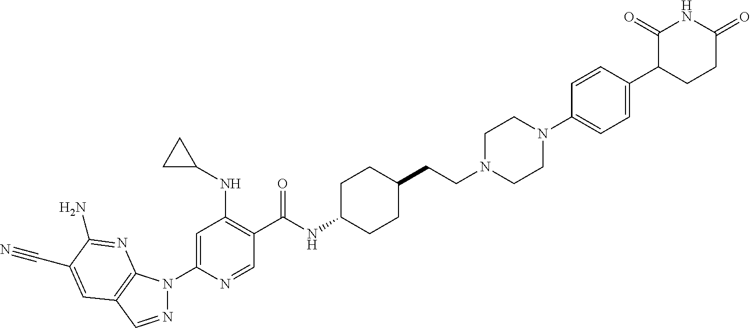 [SMILES: N#Cc1cc2cnn(-c3cc(NC4CC4)c(C(=O)N[C@H]4CC[C@H](CCN5CCN(c6ccc(C7CCC(=O)NC7=O)cc6)CC5)CC4)cn3)c2nc1N] | 23% yield as a light yellow solid. LCMS method 2: Retention time: 2.183 min, 99.9 % purity at 215 nm, [M + H]⁺ = 716.4, [M + 2H]²⁺ = 358.8. ¹H NMR (400 MHz, DMSO-d6) δ ppm 0.49-0.57 (m, 2H), 0.81-0.89 (m, 2H), 0.95-1.16 (m, 3H), 1.21-1.42 (m, 6H), 1.77-1.90 (m, 4H), 1.95-2.05 (m, 1H), 2.08-2.20 (m, 1H), 2.34-2.48 (m, 5H), 2.58-2.66 (m, 2H), 3.07-3.15 (m, 4H), 3.69-3.76 (m, 2H), 6.89 (d, J = 9.0 Hz, 2H), 7.05 (d, J = 8.8 Hz, 2H), 7.19 (br s, 2H), 7.75 (s, 1H), 8.19 (s, 1H), 8.33 (br d, J = 8.1 Hz, 1H), 8.51 (s, 1H), 8.52-8.56 (m, 2H), 10.77 (s, 1H). |
| P-132 | A51 | C12 | 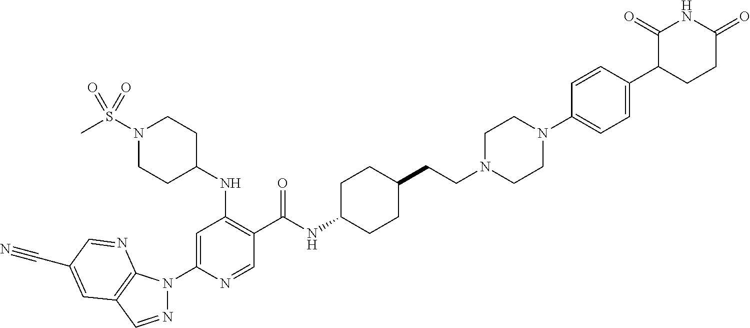 [SMILES: CS(=O)(=O)N1CCC(Nc2cc(-n3ncc4cc(C#N)cnc43)ncc2C(=O)N[C@H]2CC[C@H](CCN3CCN(c4ccc(C5CCC(=O)NC5=O)cc4)CC3)CC2)CC1] | 23% yield as an off-white solid. LCMS method 2: Retention time: 2.304 min, 96.7 % purity at 215 nm, [M + H]⁺ = 822.4, [M + 2H]²⁺ = 411.8. ¹H NMR (400 MHz, DMSO-d₆) δ ppm 0.97-1.11 (m, 2H), 1.21-1.44 (m, 6H), 1.50-1.62 (m, 2H), 1.77-1.92 (m, 4H), 1.96-2.04 (m, 1H), 2.08-2.17 (m, 3H), 2.32-2.48 (m, 4H), 2.52-2.55 (m, 2H), 2.58-2.68 (m, 1H), 2.91 (s, 3H), 2.94-3.03 (m, 2H), 3.08-3.15 (m, 4H), 3.50-3.57 (m, 2H), 3.59-3.69 (m, 1H), 3.69-3.79 (m, 2H), 6.89 (d, J = 8.8 Hz, 2H), 7.05 (d, J = 8.8 Hz, 2H), 7.40 (s, 1H), 8.40 (br d, J = 7.6 Hz, 1H), 8.61-8.69 (m, 3H), 9.02 (d, J = 2.0 Hz, 1H), 9.07 (d, J = 2.0 Hz, 1H), 10.77 (s, 1H). |
| P-133 | A52 | C12 | 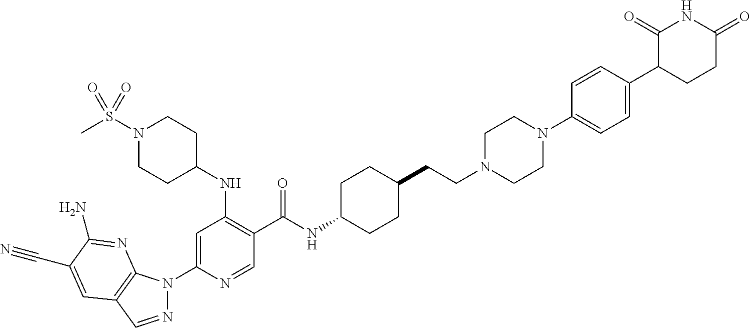 [SMILES: CS(=O)(=O)N1CCC(Nc2cc(-n3ncc4cc(C#N)c(N)nc43)ncc2C(=O)N[C@H]2CC[C@H](CCN3CCN(c4ccc(C5CCC(=O)NC5=O)cc4)CC3)CC2)CC1] | 13% yield as a light yellow solid. LCMS method 2: Retention time: 2.065 min, 99.1 % purity at 215 nm, [M + H]⁺ = 837.4, [M + 2H]²⁺ = 419.2. ¹H NMR (400 MHz, DMSO-d₆) δ ppm 0.96-1.09 (m, 2H), 1.20-1.43 (m, 6H), 1.49-1.60 (m, 2H), 1.73-1.92 (m, 5H), 1.94-2.05 (m, 1H), 2.06-2.18 (m, 3H), 2.32-2.40 (m, 2H), 2.41-2.49 (m, 4H), 2.58-2.68 (m, 2H), 3.03 (br t, J = 10.1 Hz, 2H), 3.08-3.15 (m, 4H), 3.45-3.54 (m, 2H), 3.62-3.81 (m, 4H), 6.89 (d, J = 8.8 Hz, 2H), 7.05 (d, J = 8.8 Hz, 2H), 7.20 (br s, 1H), 7.16-7.24 (m, 1H), 7.34 (s, 1H), 8.18 (s, 1H), 8.35 (br d, J = 7.6 Hz, 1H), 8.54 (s, 1H), 8.58 (s, 1H), 8.67 (br d, J = 7.6 Hz, 1H), 10.77 (s, 1H). |
| P-134 | A53 | C12 | 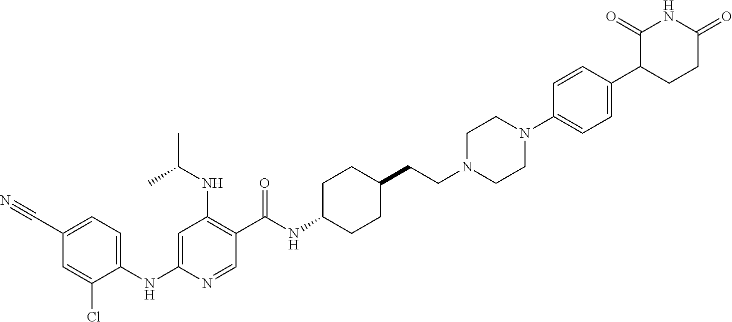 | 42% yield as a white solid. LCMS method 2: Retention time: 2.217 min, 99.6 % purity at 215 nm, [M + H]⁺ = 711.4, [M + 2H]²⁺ = 356.2. ¹H NMR (400 MHz, DMSO-d₆) δ ppm 0.93-1.07 (m, 2H), 1.20 (d, J = 6.4 Hz, 6H), 1.22-1.43 (m, 5H), 1.73-1.86 (m, 4H), 1.96-2.05 (m, 1H), 2.07-2.19 (m, 1H), 2.31-2.41 (m, 2H), 2.41-2.47 (m, 1H), 2.57-2.67 (m, 1H), 3.07-3.15 (m, 4H), 3.53-3.62 (m, 1H), 3.62-3.70 (m, 1H), 3.72 (dd, J = 11.1, 5.0 Hz, 1H), 6.54 (s, 1H), 6.89 (d, J = 8.8 Hz, 2H), 7.05 (d, J = 8.6 Hz, 2H), 7.67 (dd, J = 8.8, 2.0 Hz, 1H), 7.97 (d, J = 2.0 Hz, 1H), 8.05 (d, J = 8.1 Hz, 1H), 8.29 (br d, J = 7.3 Hz, 1H), 8.34 (s, 1H), 8.51 (d, J = 8.8 Hz, 1H), 8.64 (s, 1H), 10.77 (s, 1H). Four protons were not apparent by ¹H NMR. |
| P-135 | A54 | C12 | 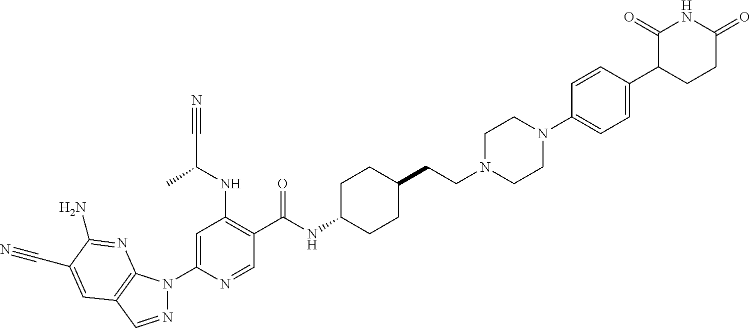 [SMILES: C[C@H](C#N)Nc1cc(-n2ncc3cc(C#N)c(N)nc32)ncc1C(=O)N[C@H]1CC[C@H](CCN2CCN(c3ccc(C4CCC(=O)NC4=O)cc3)CC2)CC1] | 24% yield as a white solid. LCMS method 2: Retention time: 2.410 min, 99.2 % purity at 215 nm, [M + H]⁺ = 729.4, [M + 2H]²⁺ = 365.3. ¹H NMR (400 MHz, DMSO-d₆) δ ppm 0.98-1.10 (m, 2H), 1.20-1.34 (m, 2H), 1.34-1.45 (m, 4H), 1.70 (d, J = 7.1 Hz, 3H), 1.77-1.85 (m, 2H), 1.86-1.93 (m, 2H), 1.96-2.06 (m, 1H), 2.07-2.19 (m, 1H), 2.33-2.39 (m, 2H), 2.40-2.49 (m, 3H), 2.58-2.67 (m, 1H), 3.12 (br s, 4H), 3.70-3.81 (m, 2H), 4.94 (quin, J = 7.0 Hz, 1H), 6.89 (d, J = 8.6 Hz, 2H), 7.06 (d, J = 8.6 Hz, 2H), 7.33 (br s, 2H), 7.89 (s, 1H), 8.23 (s, 1H), 8.48 (br d, J = 7.8 Hz, 1H), 8.57 (s, 1H), 8.66 (s, 1H), 8.78 (br d, J = 7.6 Hz, 1H), 10.77 (s, 1H). |
| P-136 | A29 | C40 | 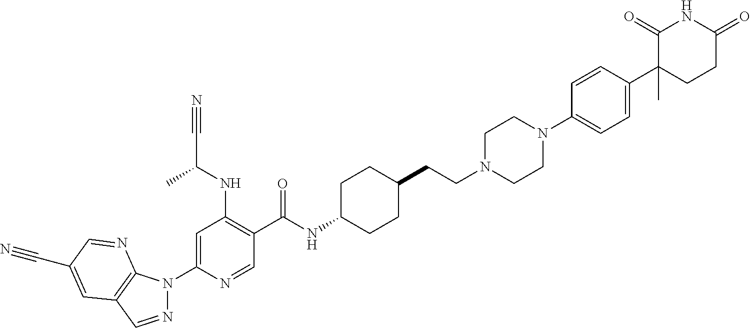 [SMILES: C[C@H](C#N)Nc1cc(-n2ncc3cc(C#N)cnc32)ncc1C(=O)N[C@H]1CC[C@H](CCN2CCN(c3ccc(C4(C)CCC(=O)NC4=O)cc3)CC2)CC1] | 13 % yield as a white solid. LCMS method 2: Retention time: 2.345 min, 94.7 % purity at 215 nm, [M + H]⁺ = 728.4, [M + 2H]²⁺ = 364.8. ¹H NMR (400 MHz, DMSO-d₆) δ ppm 0.93-1.12 (m, 3H), 1.23-1.43 (m, 8H), 1.67 (d, J = 6.8 Hz, 3H), 1.81 (br d, J = 12.0 Hz, 2H), 1.89 (s, 2H), 2.05 (q, J = 10.8 Hz, 2H), 2.23-2.49 (m, 7H), 3.07-3.15 (m, 4H), 3.75 (tdt, J = 11.5, 7.8, 3.8 Hz, 1H), 4.94 (quin, J = 7.0 Hz, 1H), 6.91 (d, J = 8.8 Hz, 2H), 7.10 (d, J = 8.8 Hz, 2H), 7.48 (s, 1H), 8.54 (d, J = 7.6 Hz, 1H), 8.69 (s, 1H), 8.71 (s, 1H), 8.77 (d, J = 7.3 Hz, 1H), 9.01-9.08 (m, 2H), 10.84 (s, 1H). |
| P-137 | A55 | C12 | 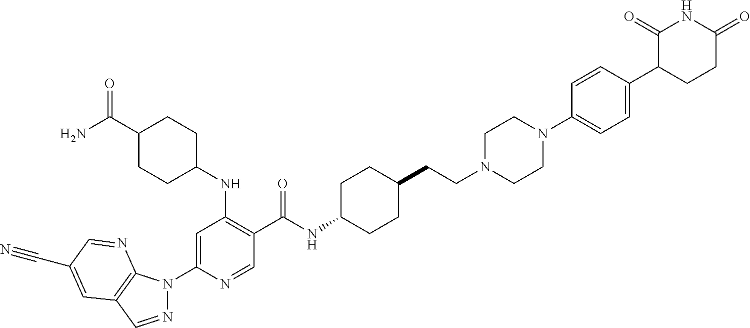 [SMILES: N#Cc1cnc2c(cnn2-c2cc(NC3CCC(C(N)=O)CC3)c(C(=O)N[C@H]3CC[C@H](CCN4CCN(c5ccc(C6CCC(=O)NC6=O)cc5)CC4)CC3)cn2)c1] | 30 % yield as an off-white solid. LCMS method 2: Retention time: 2.051 min, 99.2 % purity at 215 nm, [M + H]⁺ = 786.4, [M + 2H]²⁺ = 393.8. ¹H NMR (400 MHz, DMSO-d₆) δ ppm 0.97-1.07 (m, 2H), 1.20-1.42 (m, 8H), 1.46-1.57 (m, 2H), 1.75-1.92 (m, 7H), 1.97-2.04 (m, 1H), 2.06-2.19 (m, 5H), 2.32-2.38 (m, 2H), 2.41-2.47 (m, 2H), 2.56-2.69 (m, 2H), 3.07-3.13 (m, 4H), 3.72 (dd, J = 11.0, 4.9 Hz, 2H), 6.70 (s, 1H), 6.88 (d, J = 8.8 Hz, 2H), 7.04 (d, J = 8.6 Hz, 2H), 7.23 (s, 1H), 7.36 (s, 1H), 8.36 (d, J = 7.6 Hz, 1H), 8.55 (d, J = 7.3 Hz, 1H), 8.59 (s, 1H), 8.63 (s, 1H), 9.01 (d, J = 2.2 Hz, 1H), 9.04 (d, J = 2.0 Hz, 1H), 10.77 (s, 1H). |
| P-138 | A56 | C12 | 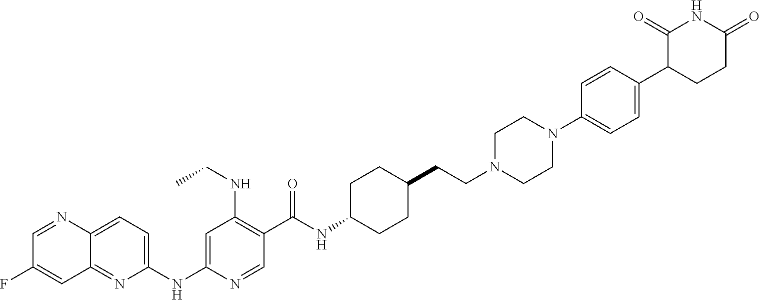 | 28 % yield as a light yellow solid. LCMS method 2: Retention time: 2.006 min, 99.9 % purity at 215 nm, [M − HCOOH + H]⁺ = 708.4, [M − HCOOH + 2H]²⁺ = 354.8. ¹H NMR (400 MHz, DMSO-d₆) δ ppm 0.98-1.13 (m, 2H), 1.25-1.36 (m, 5H), 1.37-1.48 (m, 3H), 1.82 (br d, J = 11.7 Hz, 2H), 1.89 (br d, J = 10.5 Hz, 2H), 2.03-2.18 (m, 2H), 2.39 (t, J = 7.3 Hz, 2H), 2.43-2.48 (m, 1H), 2.51-2.57 (m, 5H), 2.57-2.68 (m, 1H), 3.10-3.18 (m, 4H), 3.27-3.38 (m, 2H), 3.73 (dd, J = 9.5, 5.5 Hz, 2H), 6.88 (d, J = 8.6 Hz, 2H), 7.06 (d, J = 8.6 Hz, 2H), 7.77 (d, J = 9.3 Hz, 1H), 7.79-7.85 (m, 2H), 7.86 (s, 1H), 8.14 (br s, 1H), 8.18 (d, J = 9.2 Hz, 1H), 8.33 (t, J = 5.0 Hz, 1H), 8.39 (s, 1H), 8.70 (d, J = 2.4 Hz, 1H), 9.94 (br s, 1H), 10.45 (br s, 1H). ¹⁹NMR (377 MHz, DMSO-d₆) δ ppm −124.87 (s, 1F). |
| P-141 | A59 | C12 | 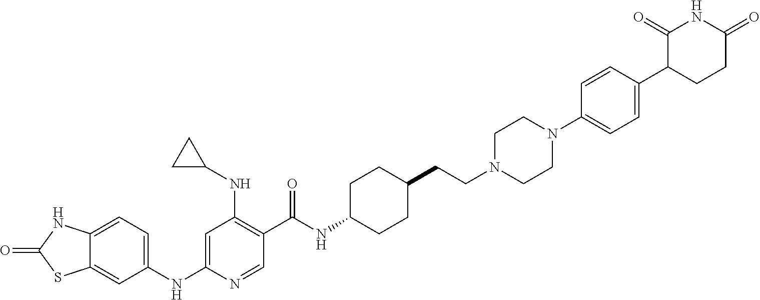 [SMILES: O=C1CCC(c2ccc(N3CCN(CC[C@H]4CC[C@H](NC(=O)c5cnc(Nc6ccc7[nH]c(=O)sc7c6)cc5NC5CC5)CC4)CC3)cc2)C(=O)N1] | 15 % yield as a white solid. LCMS method 2: Retention time: 1.972 min, 96.5 % purity at 215 nm, [M + H]⁺ = 723.3, [M + 2H]²⁺ = 362.2. ¹H NMR (400 MHz, DMSO-d₆) δ ppm 0.44-0.52 (m, 2H), 0.72-0.80 (m, 2H), 0.93-1.07 (m, 2H), 1.20-1.43 (m, 6H), 1.73-1.85 (m, 4H), 1.96-2.05 (m, 1H), 2.06-2.20 (m, 1H), 2.30-2.46 (m, 5H), 2.57-2.66 (m, 1H), 3.07-3.14 (m, 4H), 3.60-3.69 (m, 1H), 3.74 (dd, J = 1.0 Hz, 1H), 6.28 (s, 1H), 6.89 (d, J = 8.8 Hz, 2H), 6.98-7.08 (m, 3H), 7.33-7.39 (m, 1H), 7.90-7.96 (m, 1H), 8.01 (s, 1H), 8.20 (s, 1H), 8.32 (br s, 2H), 9.04 (s, 1H), 10.77 (s, 1H). Two protons were not apparent by ¹H NMR. |
| P-142 | A54 | C43 | 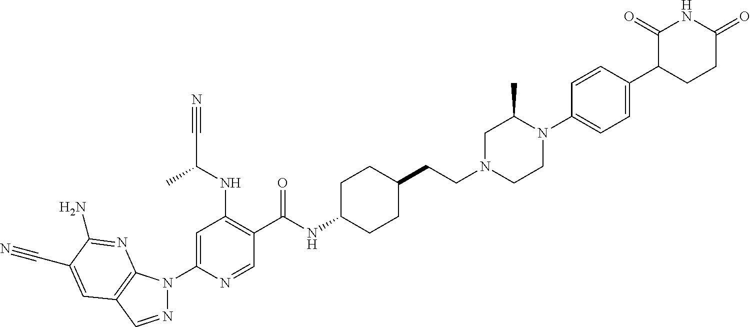 [SMILES: C[C@H](C#N)Nc1cc(-n2ncc3cc(C#N)c(N)nc32)ncc1C(=O)N[C@H]1CC[C@H](CCN2CCN(c3ccc(C4CCC(=O)NC4=O)cc3)[C@H](C)C2)CC1] | 31 % yield as a light yellow solid. LCMS method 2: Retention time: 2.259 min, 98.4 % purity at 215 nm, [M + H]⁺ = 743.4, [M + 2H]²⁺ = 372.3. ¹H NMR (400 MHz, DMSO-d₆) δ ppm 1.01 (br d, J = 6.4 Hz, 3H), 1.03-1.10 (m, 1H), 1.27-1.45 (m, 5H), 1.69 (br d, J = 6.6 Hz, 3H), 1.77-1.95 (m, 4H), 1.96-2.18 (m, 3H), 2.20-2.40 (m, 3H), 2.57-2.74 (m, 4H), 2.80-2.88 (m, 1H), 2.89-2.99 (m, 1H), 3.66-3.84 (m, 3H), 3.89-4.01 (m, 1H), 4.87-5.00 (m, 1H), 6.83 (br d, J = 8.6 Hz, 2H), 7.04 (br d, J = 8.3 Hz, 2H), 7.33 (br s, 2H), 7.88 (s, 1H), 8.22 (s, 1H), 8.48 (br d, J = 7.1 Hz, 1H), 8.56 (s, 1H), 8.66 (s, 1H), 8.79 (br d, J = 7.1 Hz, 1H), 10.77 (s, 1H). |
| P-143 | A29 | C25 | 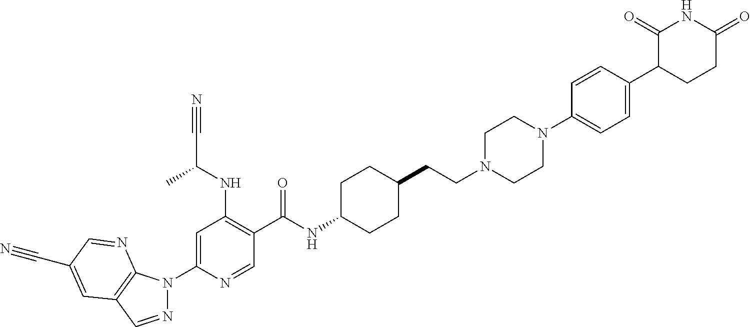 [SMILES: C[C@H](C#N)Nc1cc(-n2ncc3cc(C#N)cnc32)ncc1C(=O)N[C@H]1CC[C@H](CCN2CCN(c3ccc(C4CCC(=O)NC4=O)cc3)CC2)CC1] | 4.6 % yield as a white solid. LCMS method 2: Retention time: 2.344 min, 99.9 % purity at 215 nm, [M + H]⁺ = 713.4, [M + 2H]²⁺ = 357.3. ¹H NMR (400 MHz, DMSO-d₆) δ ppm 0.97-1.09 (m, 2H), 1.21-1.43 (m, 5H), 1.57-1.70 (m, 5H), 1.70-1.77 (m, 2H), 1.77-1.85 (m, 2H), 1.86-2.06 (m, 5H), 2.11-2.23 (m, 1H), 2.31-2.38 (m, 2H), 2.47 (br s, 2H), 2.60-2.71 (m, 1H), 2.94-3.01 (m, 2H), 3.69-3.78 (m, 1H), 3.81 (dd, J = 11.5, 5.1 Hz, 1H), 4.94 (quin, J = 6.9 Hz, 1H), 7.13 (d, J = 8.2 Hz, 2H), 7.21 (d, J = 8.2 Hz, 2H), 7.48 (s, 1H), 8.55 (d, J = 7.6 Hz, 1H), 8.69 (s, 1H), 8.71 (s, 1H), 8.77 (br d, J = 7.3 Hz, 1H), 9.03-9.06 (m, 2H), 10.81 (s, 1H). |
| P-144 | A60 | C12 | 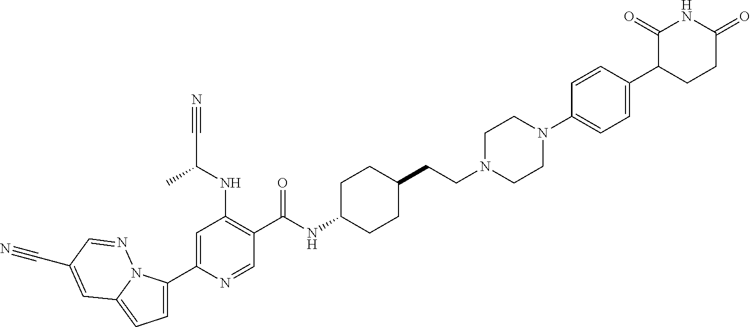 [SMILES: C[C@H](C#N)Nc1cc(-c2ccc3cc(C#N)cnn23)ncc1C(=O)N[C@H]1CC[C@H](CCN2CCN(c3ccc(C4CCC(=O)NC4=O)cc3)CC2)CC1] | 26 % yield as a yellow solid. LCMS method 2: Retention time: 2.042 min, 99.9 % purity at 215 nm, [M + H]⁺ = 713.4, [M + 2H]²⁺ = 357.2. ¹H NMR (400 MHz, DMSO-d₆) δ ppm 0.95-1.11 (m, 3H), 1.13-1.47 (m, 7H), 1.70 (d, J = 6.8 Hz, 3H), 1.75-1.84 (m, 2H), 1.85-1.93 (m, 2H), 1.94-2.06 (m, 1H), 2.06-2.21 (m, 1H), 2.31-2.41 (m, 2H), 2.41-2.48 (m, 2H), 2.58-2.68 (m, 1H), 3.08-3.15 (m, 4H), 3.69-3.77 (m, 2H), 4.80-4.93 (m, 1H), 6.89 (d, J = 8.6 Hz, 2H), 7.05 (d, J = 8.6 Hz, 2H), 7.13 (d, J = 4.9 Hz, 1H), 7.86 (d, J = 4.6 Hz, 1H), 8.22 (s, 1H), 8.45 (d, J = 7.6 Hz, 1H), 8.66 (d, J = 2.2 Hz, 1H), 8.73 (d, J = 6.8 Hz, 1H), 8.78 (s, 1H), 8.87 (d, J = 2.0 Hz, 1H), 10.77 (s, 1H). |
| P-145 | A61 | C12 | 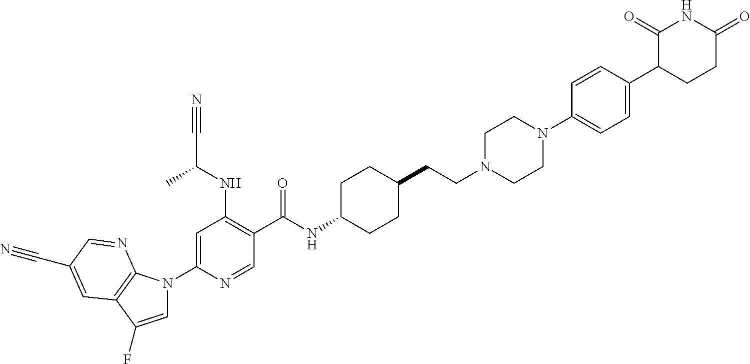 [SMILES: C[C@H](C#N)Nc1cc(-n2cc(F)c3cc(C#N)cnc32)ncc1C(=O)N[C@H]1CC[C@H](CCN2CCN(c3ccc(C4CCC(=O)NC4=O)cc3)CC2)CC1] | 24 % yield as a white solid. LCMS method 2: Retention time: 2.623 min, 98.5 % purity at 215 nm, [M + H]⁺ = 731.3, [M + 2H]²⁺ = 366.3. ¹H NMR (400 MHz, DMSO-d₆) δ ppm 0.96-1.11 (m, 2H), 1.21-1.45 (m, 6H), 1.71 (d, J = 6.8 Hz, 3H), 1.76-1.93 (m, 5H), 1.96-2.05 (m, 1H), 2.06-2.19 (m, 1H), 2.35 (br dd, J = 7.9, 7.2 Hz, 2H), 2.41-2.47 (m, 1H), 2.56-2.71 (m, 2H), 3.11 (br s, 4H), 3.69-3.79 (m, 2H), 4.81-4.91 (m, 1H), 6.89 (br d, J = 8.8 Hz, 2H), 7.05 (d, J = 8.8 Hz, 2H), 8.28 (s, 1H), 8.49 (br d, J = 7.6 Hz, 1H), 8.53 (d, J = 1.5 Hz, 1H), 8.66 (s, 1H), 8.84-8.93 (m, 3H), 10.77 (s, 1H). ¹⁹NMR (377 MHz, DMSO-d₆) δ ppm −169.27 (s, 1F). One proton was not apparent by ¹H NMR. |
| P-146 | A-54 | C25 | 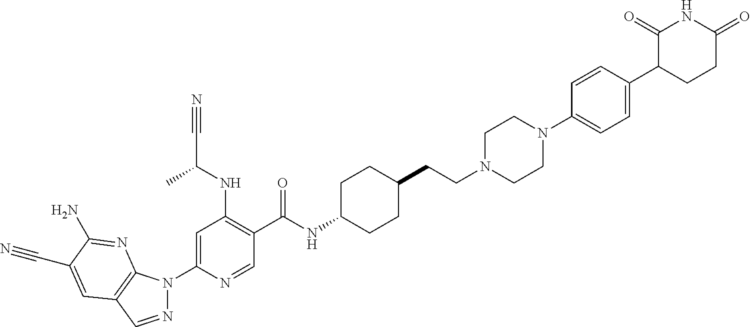 [SMILES: C[C@H](C#N)Nc1cc(-n2ncc3cc(C#N)c(N)nc32)ncc1C(=O)N[C@H]1CC[C@H](CCN2CCN(c3ccc(C4CCC(=O)NC4=O)cc3)CC2)CC1] | 12 % yield as a yellow solid. LCMS method 5: Retention time: 2.311 min, 99.0 % purity at 215 nm, [M − HCOOH + H]⁺ = 728.3, [M − HCOOH + 2H]²⁺ = 364.8. ¹H NMR (400 MHz, DMSO-d₆) δ ppm 0.97-1.07 (m, 2H), 1.18-1.44 (m, 6H), 1.59-1.83 (m, 9H), 1.84-1.93 (m, 2H), 1.97-2.06 (m, 3H), 2.12-2.21 (m, 1H), 2.34-2.40 (m, 2H), 2.43-2.47 (m, 1H), 2.60-2.67 (m, 1H), 3.00 (br d, J = 11.0 Hz, 2H), 3.70-3.77 (m, 1H), 3.81 (br dd, J = 11.9, 4.8 Hz, 1H), 4.90-4.97 (m, 1H), 7.11-7.16 (m, 2H), 7.18-7.23 (m, 2H), 7.33 (br s, 2H), 7.89 (s, 1H), 8.18 (s, 1H), 8.23 (s, 1H), 8.49 (br d, J = 7.6 Hz, 1H), 8.56 (s, 1H), 8.66 (s, 1H), 8.77 (br d, J = 7.3 Hz, 1H), 10.81 (s, 1H). One proton was not apparent by ¹H NMR. |
| P-147 | A62 | C12 | 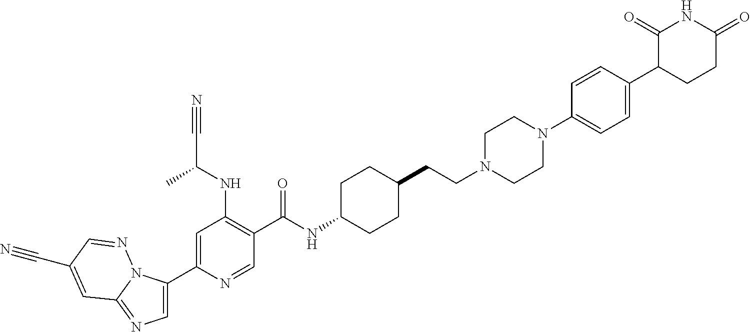 [SMILES: C[C@H](C#N)Nc1cc(-c2cnc3cc(C#N)cnn23)ncc1C(=O)N[C@H]1CC[C@H](CCN2CCN(c3ccc(C4CCC(=O)NC4=O)cc3)CC2)CC1] | 22 % yield as a light yellow solid. LCMS method 2: Retention time: 2.350 min, 99.9 % purity at 215 nm, [M + H]⁺ = 714.4, [M + 2H]²⁺ = 357.8. ¹H NMR (400 MHz, DMSO-d₆) δ ppm 0.93-1.13 (m, 2H), 1.20-1.35 (m, 3H), 1.36-1.48 (m, 3H), 1.70 (d, J = 6.8 Hz, 3H), 1.75-1.84 (m, 2H), 1.85-1.94 (m, 2H), 1.95-2.06 (m, 1H), 2.06-2.21 (m, 1H), 2.36 (br t, J = 7.3 Hz, 2H), 2.42-2.49 (m, 2H), 2.57-2.73 (m, 1H), 3.02-3.20 (m, 4H), 3.72 (dd, J = 10.9, 4.8 Hz, 1H), 3.75-3.84 (m, 1H), 4.88 (quin, J = 6.9 Hz, 1H), 6.89 (d, J = 8.7 Hz, 2H), 7.05 (d, J = 8.4 Hz, 2H), 8.08 (s, 1H), 8.49 (br d, J = 7.6 Hz, 1H), 8.72 (s, 1H), 8.78 (br d, J = 7.6 Hz, 1H), 8.80 (s, 1H), 9.05 (d, J = 1.8 Hz, 1H), 9.12 (d, J = 2.0 Hz, 1H), 10.77 (s, 1H). Two protons were not apparent by ¹H NMR. |
| P-148 | A62 | C25 | 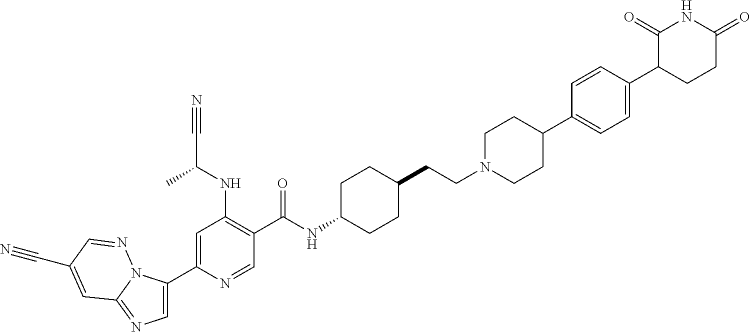 [SMILES: C[C@H](C#N)Nc1cc(-c2cnc3cc(C#N)cnn23)ncc1C(=O)N[C@H]1CC[C@H](CCN2CCC(c3ccc(C4CCC(=O)NC4=O)cc3)CC2)CC1] | 15 % yield as a light yellow solid. LCMS method 2: Retention time: 2.424 min, 99.9 % purity at 215 nm, [M + H]⁺ = 713.4, [M + 2H]²⁺ = 357.3. 1H NMR (400 MHz, DMSO-d₆) δ ppm 0.95-1.10 (m, 2H), 1.21-1.29 (m, 1H), 1.30-1.48 (m, 4H), 1.57-1.68 (m, 2H), 1.71 (d, J = 7.0 Hz, 3H), 1.72-1.84 (m, 4H), 1.84-1.94 (m, 2H), 1.94-2.10 (m, 3H), 2.10-2.26 (m, 1H), 2.36 (br t, J = 7.2 Hz, 2H), 2.42-2.49 (m, 2H), 2.57-2.74 (m, 1H), 2.99 (br d, J = 10.9 Hz, 2H), 3.67-3.78 (m, 1H), 3.81 (dd, J = 11.4, 4.9 Hz, 1H), 4.89 (quin, J = 6.9 Hz, 1H), 7.13 (d, J = 8.2 Hz, 2H), 7.20 (d, J = 8.2 Hz, 2H), 8.09 (s, 1H), 8.50 (br d, J = 7.7 Hz, 1H), 8.72 (s, 1H), 8.78 (br d, J = 7.2 Hz, 1H), 8.80 (s, 1H), 9.05 (d, J = 1.8 Hz, 1H), 9.12 (d, J = 2.0 Hz, 1H), 10.82 (s, 1H). |
| P-149 | A29 | C31 | 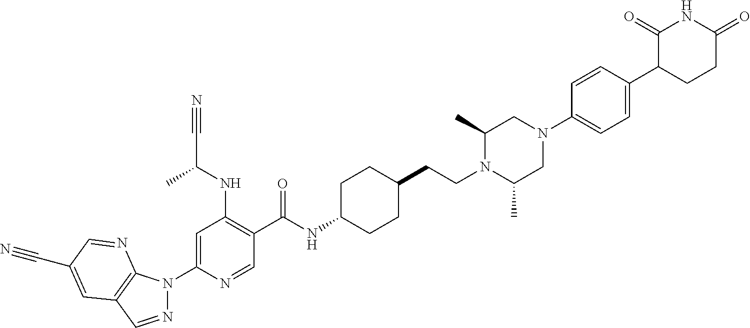 [SMILES: C[C@H](C#N)Nc1cc(-n2ncc3cc(C#N)cnc32)ncc1C(=O)N[C@H]1CC[C@H](CCN2[C@@H](C)CN(c3ccc(C4CCC(=O)NC4=O)cc3)C[C@@H]2C)CC1] | 25% yield as an off-white solid. LCMS method 7: Retention time: 1.736 min, 97.0% purity at 215 nm, [M + H]⁺ = 742.0, [M + 2H]²⁺ = 371.6. ¹H-NMR (400 MHz, DMSO-d₆): δ ppm 1.03 (d, J = 6.0 Hz, 6H), 1.03-1.07 (m, 1H), 1.32-1.41 (m, 5H), 1.68 (d, J = 6.8 Hz, 3H), 1.79-1.90 (m, 4H), 1.99-2.02 (m, 1H), 2.12-2.18 (m, 1H), 2.28-2.33 (m, 1H), 2.59-2.75 (m, 4H), 2.84-2.88 (m, 2H), 2.98-3.01 (m, 2H), 3.11-3.13 (m, 2H), 3.70-3.76 (m, 2H), 4.94-4.98 (m, 1H), 6.87 (d, J = 8.8 Hz, 2H), 7.04 (d, J = 8.8 Hz, 2H), 7.48 (s, 1H), 8.55 (d, J = 8.0 Hz, 1H), 8.70 (s, 1H), 8.72 (s, 1H), 8.78 (d, J = 7.6 Hz, 1H), 9.04-9.06 (m, 2H), 10.77 (s, 1H). |
| P-150 | A29 | C43 | 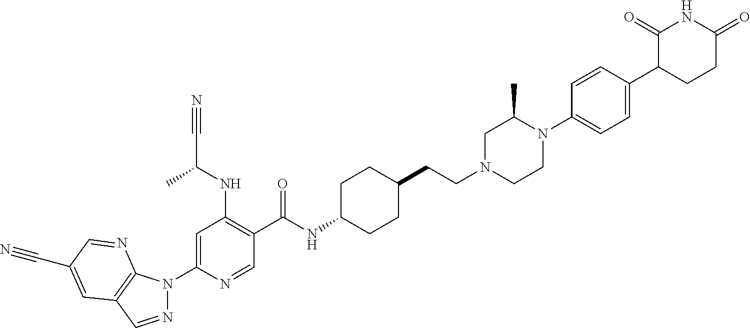 [SMILES: C[C@H](C#N)Nc1cc(-n2ncc3cc(C#N)cnc32)ncc1C(=O)N[C@H]1CC[C@H](CCN2CCN(c3ccc(C4CCC(=O)NC4=O)cc3)[C@H](C)C2)CC1] | 14 % yield as an off-white solid. LCMS method 7: Retention time: 1.673 min, 96.9 % purity at 215 nm, [M + H]⁺ = 728.0. ¹H-NMR (400 MHz, DMSO-d₆): δ ppm 1.01 (d, J = 6.4 Hz, 3H), 1.01-1.06 (m, 2H), 1.33-1.41 (m, 5H), 1.68 (d, J = 6.8 Hz, 3H), 1.81-1.90 (m, 4H), 2.01-2.14 (m, 3H), 2.24-2.34 (m, 3H), 2.60-2.71 (m, 2H), 2.84-2.94 (m, 2H), 3.25-3.29 (m, 2H), 3.70-3.74 (m, 2H), 3.94-3.96 (m, 1H), 4.92-4.96 (m, 1H), 6.84 (d, J = 8.8 Hz, 2H), 7.04 (d, J = 8.4 Hz, 2H), 7.48 (s, 1H), 8.55 (d, J = 7.6 Hz, 1H), 8.70 (s, 1H), 8.71 (s, 1H), 8.79 (d, J = 7.2 Hz, 1H), 9.04-9.06 (m, 2H), 10.78 (s, 1H). |
| P-151 | A29 | C48 | 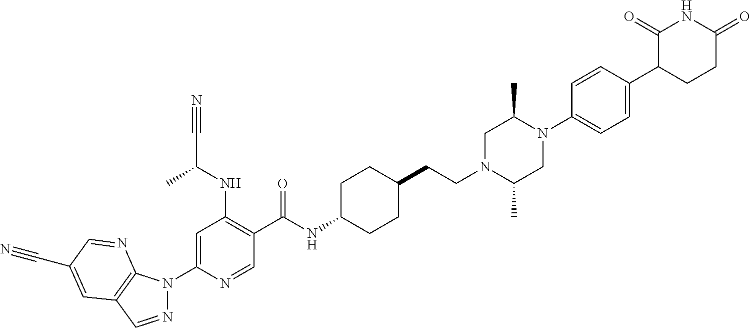 [SMILES: C[C@H](C#N)Nc1cc(-n2ncc3cc(C#N)cnc32)ncc1C(=O)N[C@H]1CC[C@H](CCN2C[C@@H](C)N(c3ccc(C4CCC(=O)NC4=O)cc3)C[C@@H]2C)CC1] | 16 % yield as an off-white solid. LCMS method 7: Retention time: 1.417 min, 93.6 % purity at 215 nm, [M + H]⁺ = 742.2, [M + 2H]²⁺ = 371.8. ¹H-NMR (400 MHz, DMSO-d₆): δ ppm 0.97 (d, J = 6.4 Hz, 6H), 0.97-1.06 (m, 2H), 1.36-1.42 (m, 5H), 1.67 (d, J = 6.8 Hz, 3H), 1.81-1.91 (m, 4H), 2.03-2.24 (m, 3H), 2.60-2.65 (m, 1H), 2.79-2.89 (m, 3H), 3.06-3.08 (m, 1H), 3.15-3.18 (m, 2H), 3.55-3.59 (m, 1H), 3.73-3.78 (m, 2H), 4.09-4.12 (m, 1H), 4.92-4.97 (m, 1H), 6.92 (d, J = 8.4 Hz, 2H), 7.07 (d, J = 8.8 Hz, 2H), 7.49 (s, 1H), 8.55 (d, J = 7.6 Hz, 1H), 8.70 (s, 1H), 8.72 (s, 1H), 8.79 (d, J = 7.6 Hz, 1H), 9.04-9.06 (m, 2H), 10.79 (s, 1H). |
| P-152 | A29 | C49 | 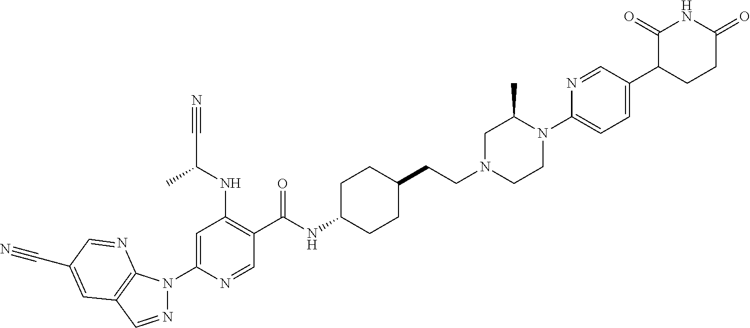 [SMILES: C[C@H](C#N)Nc1cc(-n2ncc3cc(C#N)cnc32)ncc1C(=O)N[C@H]1CC[C@H](CCN2CCN(c3ccc(C4CCC(=O)NC4=O)cn3)[C@H](C)C2)CC1] | 27% yield as an off-white solid. LCMS method 7: Retention time: 1.259 min, 98.0% purity at 215 nm, [M + H]⁺ = 729.4, [M + 2H]²⁺ = 365.2. ¹H NMR (400 MHz, DMSO-d₆): δ ppm 1.03-1.11 (m, 2H), 1.13 (d, J = 6.8 Hz, 3H), 1.36-1.42 (m, 5H), 1.68 (d, J = 6.8 Hz, 3H), 1.82-1.97 (m, 6H), 2.09-2.20 (m, 2H), 2.28-2.41 (m, 3H), 2.65-2.74 (m, 1H), 2.79-2.81 (m, 1H), 2.95-3.02 (m, 2H), 3.70-3.75 (m, 2H), 3.91-3.95 (m, 1H), 4.39-4.49 (m, 1H), 4.89-4.92 (m, 1H), 6.72 (d, J = 8.8 Hz, 1H), 7.37 (dd, J = 2.4 Hz and 8.8 Hz, 1H), 7.49 (s, 1H), 7.95 (d, J = 2.4 Hz, 1H), 8.54 (d, J = 8.0 Hz, 1H), 8.70 (s, 1H), 8.72 (s, 1H), 8.79 (d, J = 7.6 Hz, 1H), 9.04-9.06 (m, 2H), 10.80 (s, 1H). |
| P-153 | A29 | C50 | 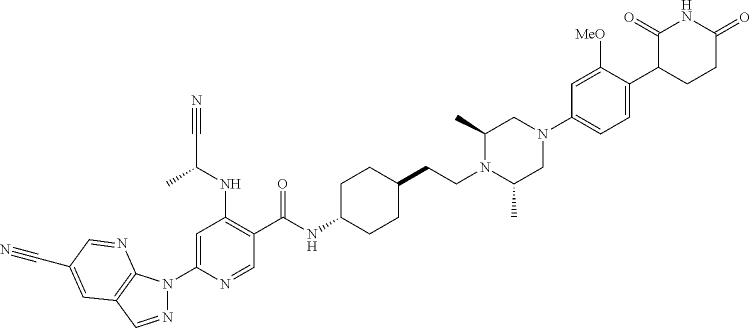 [SMILES: COc1cc(N2C[C@H](C)N(CC[C@H]3CC[C@H](NC(=O)c4cnc(-n5ncc6cc(C#N)cnc65)cc4N[C@H](C)C#N)CC3)[C@@H](C)C2)ccc1C1CCC(=O)NC1=O] | 1.5% yield as an off-white solid. LCMS method 8: Retention time: 1.809 min, 97.0% purity at 215 nm, [M + H]⁺ = 772.3, [M + 2H]²⁺ = 386.8. ¹H-NMR (400 MHz, DMSO-d₆): δ ppm 1.04 (d, J = 6.4 Hz, 6H), 1.04-1.11 (m, 1H), 1.16-1.38 (m, 5H), 1.67 (d, J = 7.2 Hz, 3H), 1.79-1.95 (m, 6H), 2.15-2.25 (m, 2H), 2.45-2.49 (m, 1H), 2.61-2.67 (m, 1H), 2.70-2.78 (m, 1H), 2.86-2.91 (m, 2H), 3.00-3.05 (m, 2H), 3.18-3.18 (m, 2H), 3.77 (s, 3H), 3.78-3.80 (m, 1H), 4.93-4.96 (m, 1H), 6.42-6.45 (m, 1H), 6.51 (s, 1H), 6.91 (d, J = 8.40 Hz, 1H), 7.48 (s, 1H), 8.54-8.56 (m, 1H), 8.69 (s, 1H), 8.71 (s, 1H), 8.77 (d, J = 7.2 Hz, 1H), 9.04-9.06 (m, 2H), 10.67 (s, 1H). One proton was not apparent by ¹H NMR. |
| P-158 | A29 | C51 | 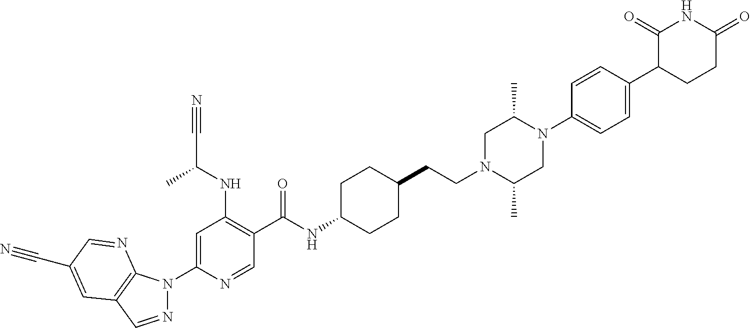 [SMILES: C[C@H](C#N)Nc1cc(-n2ncc3cc(C#N)cnc32)ncc1C(=O)N[C@H]1CC[C@H](CCN2C[C@H](C)N(c3ccc(C4CCC(=O)NC4=O)cc3)C[C@@H]2C)CC1] | 9.4% yield as an off-white solid. LCMS method 7: Retention time: 1.877 min, 99.3% purity at 215 nm, [M + H]⁺ = 742.3, [M + 2H]²⁺ = 371.8. ¹H NMR (400 MHz, DMSO-d₆): δ ppm 1.02 (d, J = 6.4 Hz, 3H), 1.11 (d, J = 6.0 Hz, 3H), 1.32-1.41 (m, 5H), 1.67 (d, J = 6.8 Hz, 3H), 1.77-1.99 (m, 5H), 2.00-2.13 (m, 3H), 2.27-2.33 (m, 2H), 2.83-2.85 (m, 2H), 3.70-3.80 (m, 2H), 3.98-4.02 (m, 1H), 4.92-4.96 (m, 1H), 6.81 (d, J = 8.8 Hz, 2H), 7.03 (d, J = 8.8 Hz, 2H), 7.49 (s, 1H), 8.56 (d, J = 8.0 Hz, 1H), 8.69 (s, 1H), 8.71 (s, 1H), 8.79 (d, J = 7.2 Hz, 1H), 9.03-9.05 (m, 2H), 10.76 (s, 1H). Five protons were not apparent by ¹H NMR. |
| P-159 | A65 | C12 | 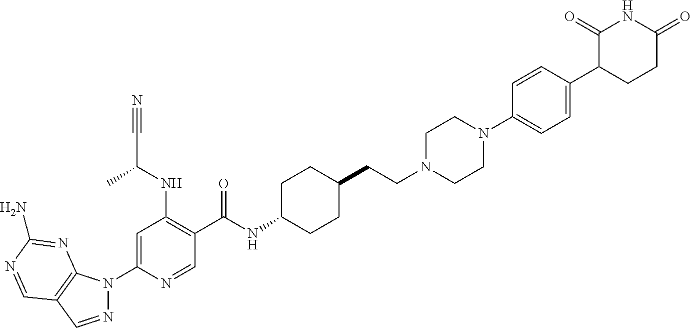 [SMILES: C[C@H](C#N)Nc1cc(-n2ncc3cnc(N)nc32)ncc1C(=O)N[C@H]1CC[C@H](CCN2CCN(c3ccc(C4CCC(=O)NC4=O)cc3)CC2)CC1] | 4.1% yield as an off-white solid. LCMS method 8: Retention time: 1.086 min, 98.2% purity at 215 nm, [M + H]⁺ = 705.3, [M + 2H]²⁺ = 353.3. ¹H NMR (400 MHz, DMSO-d₆): δ ppm 1.05-1.15 (m, 2H), 1.33-1.42 (m, 3H), 1.61-1.63 (m, 2H), 1.70 (d, J = 6.80 Hz, 3H), 1.81-1.84 (m, 2H), 1.92-2.03 (m, 3H), 2.14-2.19 (m, 1H), 2.66-2.68 (m, 1H), 2.97 (t, J = 12.00 Hz, 2H), 3.13-3.22 (m, 4H), 3.59-3.62 (m, 2H), 3.75-3.90 (m, 4H), 4.91-4.94 (m, 1H), 6.99 (d, J = 8.40 Hz, 2H), 7.13 (d, J = 8.80 Hz, 2H), 7.23 (brs, 2H), 7.88 (s, 1H), 8.30 (s, 1H), 8.56 (d, J = 7.60 Hz, 1H), 8.66 (s, 1H), 8.85 (d, J = 7.20 Hz, 1H), 8.93 (s, 1H), 10.81 (s, 1H). One proton was not apparent by ¹H NMR. |
| P-161 | A54 | C52 | 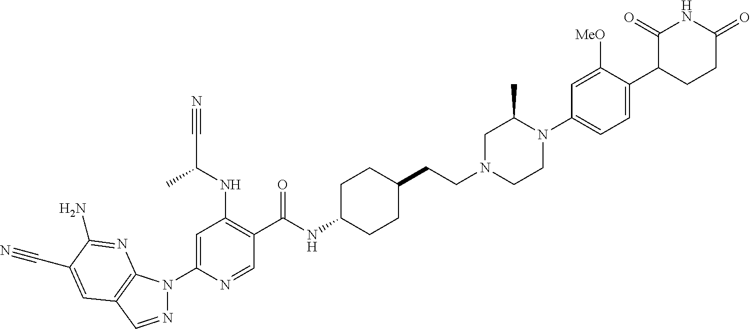 [SMILES: COc1cc(N2CCN(CC[C@H]3CC[C@H](NC(=O)c4cnc(-n5ncc6cc(C#N)c(N)nc65)cc4N[C@H](C)C#N)CC3)C[C@H]2C)ccc1C1CCC(=O)NC1=O] | 15 % yield as an off-white solid. LCMS method 8: Retention time: 1.487 min, 96.2% purity at 215 nm, [M + H]⁺ = 773.3, [M + 2H]²⁺ = 387.3. ¹H-NMR (400 MHz, DMSO-d₆, 80 ° C.): δ ppm 1.09-1.18 (m, 5H), 1.38-1.45 (m, 3H), 1.66-1.70 (m, 2H), 1.71 (d, J = 6.8 Hz, 3H), 1.84-1.87 (m, 2H), 1.92-1.97 (m, 3H), 2.10-2.19 (m, 1H), 2.61-2.68 (m, 2H), 3.45-3.60 (m, 3H), 3.77 (s, 3H), 3.80-3.84 (m, 2H), 4.93-4.96 (m, 1H), 6.66 (brs, 2H), 6.95-7.05 (m, 3H), 7.83 (s, 1H), 8.22 (s, 1H), 8.32 (d, J = 7.6 Hz, 1H), 8.52 (s, 1H), 8.68 (s, 1H), 8.74 (d, J = 6.8 Hz, 1H), 10.41 (s, 1H). Six protons were not apparent by ¹H NMR. |
| P-163 | A68 | C12 | 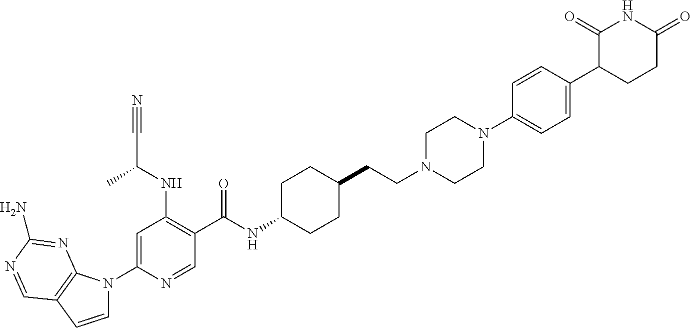 [SMILES: C[C@H](C#N)Nc1cc(-n2ccc3cnc(N)nc32)ncc1C(=O)N[C@H]1CC[C@H](CCN2CCN(c3ccc(C4CCC(=O)NC4=O)cc3)CC2)CC1] | 17% yield as an off-white solid. LCMS method 8: Retention time: 1.497 min, 99.6% purity at 215 nm, [M + H]⁺ = 704.4 ¹H NMR (400 MHz, DMSO-d6): δ ppm 1.07-1.13 (m, 2H), 1.32-1.39 (m, 3H), 1.60-1.63 (m, 2H), 1.72 (d, J = 6.80 Hz, 3H), 1.80-1.83 (m, 2H), 1.89-2.03 (m, 3H), 2.14-2.18 (m, 1H), 2.61-2.64 (m, 1H), 2.93-2.99 (m, 2H), 3.11-3.22 (m, 5H), 3.75-3.86 (m, 4H), 4.90-4.94 (m, 1H), 6.68 (d, J = 4.00 Hz, 1H), 6.99 (d, J = 8.80 Hz, 2H), 7.07-7.14 (m, 4H), 8.08 (d, J = 4.00 Hz, 1H), 8.44 (s, 1H), 8.47 (t, J = 7.6 Hz, 1H), 8.65 (s, 1H), 8.69 (s, 1H), 8.82 (d, J = 7.20 Hz, 1H), 10.81 (s, 1H). Two protons were not apparent by ¹H NMR. |
| P-164 | A29 | C53 | 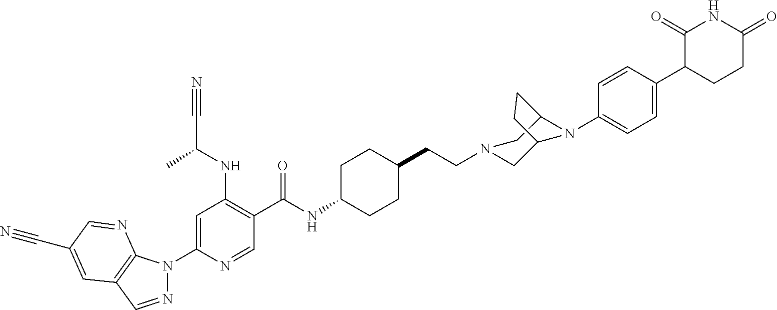 [SMILES: C[C@H](C#N)Nc1cc(-n2ncc3cc(C#N)cnc32)ncc1C(=O)N[C@H]1CC[C@H](CCN2CC3CCC(C2)N3c2ccc(C3CCC(=O)NC3=O)cc2)CC1] | 27% yield as an off-white solid. LCMS method 7: Retention time: 1.310 min, 95.0% purity at 215 nm, [M + H]⁺ = 740.2 ¹H NMR: (400 MHz, DMSO-d6): δ ppm 1.03-1.09 (m, 2H), 1.22-1.35 (m, 4H), 1.52-1.56 (m, 2H), 1.66 (d, J = 7.2 Hz, 3H), 1.74-1.77 (m, 2H), 1.87-2.16 (m, 8H), 2.60-2.65 (m, 1H), 3.05-3.08 (m, 4H), 3.35-3.39 (m, 2H), 4.44-4.46 (m, 2H), 4.92-4.98 (m, 1H), 6.89 (d, J = 8.8 Hz, 2H), 7.10 (d, J = 8.8 Hz, 2H), 7.48 (s, 1H), 8.55 (d, J = 7.6 Hz, 1H), 8.70 (s, 1H), 8.75 (d, J = 7.6 Hz, 1H), 9.03-9.05 (m, 3H), 10.81 (s, 1H). Two protons were not apparent by ¹H NMR. |
| P-165 | A54 | C54 | 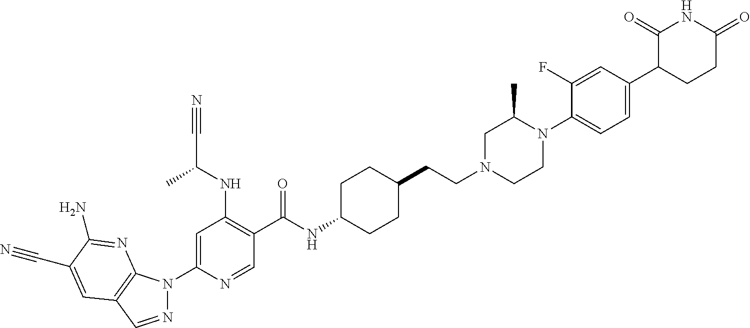 [SMILES: C[C@H](C#N)Nc1cc(-n2ncc3cc(C#N)c(N)nc32)ncc1C(=O)N[C@H]1CC[C@H](CCN2CCN(c3ccc(C4CCC(=O)NC4=O)cc3F)[C@H](C)C2)CC1] | 22% yield as an off-white solid. LCMS method 7: Retention time: 1.350 min, 98.4% purity at 215 nm, [M + H]⁺ = 761.2 ¹H NMR (400 MHz, DMSO-d6): δ ppm 0.91 (d, J = 6.0 Hz, 3H), 1.06-1.14 (m, 4H), 1.32-1.42 (m, 4H), 1.61-1.68 (m, 2H), 1.70 (d, J = 6.8 Hz, 3H), 1.80-1.84 (m, 2H), 1.89-2.06 (m, 3H), 2.15-2.28 (m, 1H), 3.06-3.26 (m, 5H), 3.36-3.42 (m, 2H), 3.67-3.55 (m, 2H), 4.91-4.98 (m, 1H), 7.03-7.29 (m, 3H), 7.40 (brs, 2H), 7.96 (s, 1H), 8.26 (s, 1H), 8.56 (d, J = 7.6 Hz, 1H), 8.59 (s, 1H), 8.67 (s, 1H), 8.85 (d, J = 6.8 Hz, 1H), 10.85 and 10.88 (s, 1H) One proton was not apparent by ¹H NMR. |
| P-166 | A54 | C55 | 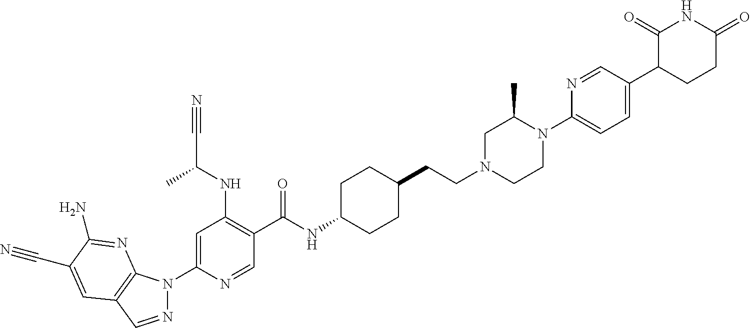 [SMILES: C[C@H](C#N)Nc1cc(-n2ncc3cc(C#N)c(N)nc32)ncc1C(=O)N[C@H]1CC[C@H](CCN2CCN(c3ccc(C4CCC(=O)NC4=O)cn3)[C@H](C)C2)CC1] | 22% yield as an off-white solid. LCMS method 7: Retention time: 1.143 min, 99.7% purity at 215 nm, [M + H]⁺ = 744.2 ¹H-NMR (400 MHz, DMSO-d6): 8 1.04-1.11 (m, 2H), 1.22 (d, J = 7.2 Hz, 3H), 1.24-1.41 (m, 3H), 1.58-1.69 (m, 2H), 1.70 (d, J = 6.8 Hz, 3H), 1.80-1.83 (m, 2H), 1.91-1.99 (m, 3H), 2.15-2.25 (m, 1H), 2.66-2.69 (m, 1H), 2.96-3.05 (m, 1H), 3.14-3.20 (m, 4H), 3.48-3.54 (m, 2H), 3.76-3.81 (m, 2H), 4.32-4.36 (m, 1H), 4.75-4.80 (m, 1H), 4.93-5.00 (m, 1H), 6.89 (d, J = 8.8 Hz, 1H), 7.42 (brs, 2H), 7.51 (dd, J = 2.0 Hz and 8.8 Hz, 1H), 7.98 (s, 1H), 8.02 (d, J = 2.4 Hz, 1H), 8.28 (s, 1H), 8.58-8.60 (m, 2H), 8.65 (s, 1H), 8.91 (d, J = 7.2 Hz, 1H), 10.84 (s, 1H). One proton was not apparent by ¹H NMR. |
| P-167 | A54 | C56 | 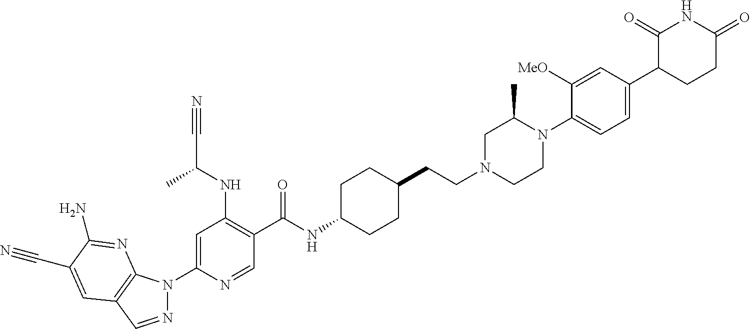 [SMILES: COc1cc(C2CCC(=O)NC2=O)ccc1N1CCN(CC[C@H]2CC[C@H](NC(=O)c3cnc(-n4ncc5cc(C#N)c(N)nc54)cc3N[C@H](C)C#N)CC2)C[C@H]1C] | 22% yield as an off-white solid. LCMS method 8: Retention time: 1.644 min, 98.8% purity at 215 nm, [M + H]⁺ = 773.4 ¹H NMR (400 MHz, DMSO-d6): δ ppm 0.85 and 0.99 (d, J = 6.0 Hz, 3H), 1.04-1.14 (m, 2H), 1.30-1.42 (m, 3H), 1.60-1.65 (m, 2H), 1.70 (d, J = 6.8 Hz, 3H), 1.80-1.84 (m, 2H), 1.89-2.05 (m, 3H), 2.18-2.26 (m, 1H), 2.75-2.83 (m, 1H), 3.10-3.24 (m, 5H), 3.78 (s, 3H), 4.94-4.99 (m, 1H), 6.75-6.88 (m, 1H), 6.92 (s, 1H), 7.04 (d, J = 8.0 Hz, 1H), 7.46 (brs, 2H), 7.97 (s, 1H), 8.27 (s, 1H), 8.56 (d, J = 7.2 Hz, 1H), 8.59 (s, 1H), 8.66 (s, 1H), 8.87 (d, J = 9.2 Hz, 1H), 10.85 (s, 1H). Seven protons were not apparent by ¹H NMR. |
| P-168 | A54 | C57 | 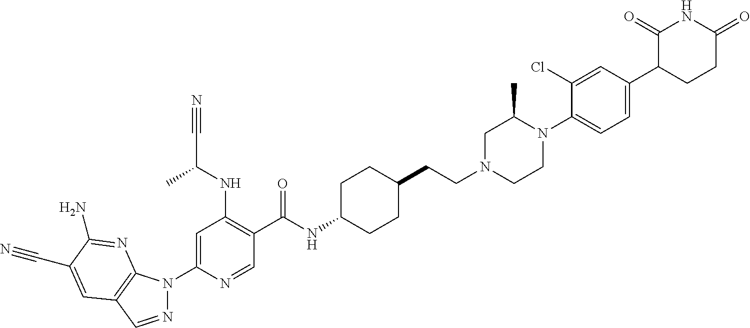 [SMILES: C[C@H](C#N)Nc1cc(-n2ncc3cc(C#N)c(N)nc32)ncc1C(=O)N[C@H]1CC[C@H](CCN2CCN(c3ccc(C4CCC(=O)NC4=O)cc3Cl)[C@H](C)C2)CC1] | 3.6% yield as an off-white solid. LCMS method 7: Retention time: 1.491 min, 96.3% purity at 215 nm, [M + H]⁺ = 777.2 ¹H NMR (400 MHz, DMSO-d6): δ ppm 0.85 (d, J = 6.0 Hz, 3H), 0.99-1.08 (m, 2H), 1.30-1.41 (m, 5H), 1.70 (d, J = 6.8 Hz, 3H), 1.80-1.91 (m, 4H), 1.99-2.23 (m, 3H), 2.33-2.39 (m, 3H), 2.62-2.69 (m, 4H), 2.72-2.78 (m, 1H), 3.10-3.15 (m, 1H), 3.72-3.88 (m, 2H), 4.91-4.98 (m, 1H), 7.16-7.24 (m, 2H), 7.31 (d, J = 1.6 Hz, 1H), 7.34 (brs, 2H), 7.89 (s, 1H), 8.23 (s, 1H), 8.50 (d, J = 8.0 Hz, 1H), 8.57 (s, 1H), 8.66 (s, 1H), 8.78 (d, J = 7.2 Hz, 1H), 10.85 (s, 1H). One proton was not apparent by ¹H NMR. |
| P-170 | A54 | C58 | 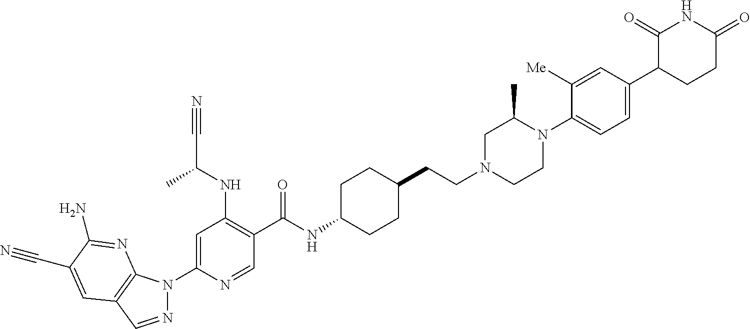 [SMILES: Cc1cc(C2CCC(=O)NC2=O)ccc1N1CCN(CC[C@H]2CC[C@H](NC(=O)c3cnc(-n4ncc5cc(C#N)c(N)nc54)cc3N[C@H](C)C#N)CC2)C[C@H]1C] | 35% yield as an off-white solid. LCMS method 8: Retention time: 1.736 min, 98.6% purity at 215 nm, [M + H]⁺ = 757.4 ¹H NMR (400 MHz, DMSO-d6): 8 0.81 (d, J = 6.0 Hz, 3H), 1.07-1.15 (m, 2H), 1.32-1.40 (m, 3H), 1.60-1.66 (m, 2H), 1.71 (d, J = 6.8 Hz, 3H), 1.81-1.84 (m, 2H), 1.88-1.94 (m, 2H), 2.03-2.06 (m, 1H), 2.13-2.20 (m, 1H), 2.26 (s, 3H), 2.64-2.68 (m, 1H), 2.79-2.86 (m, 2H), 2.98-3.02 (m, 1H), 3.12-3.28 (m, 4H), 3.33-3.37 (m, 2H), 3.76-3.83 (m, 3H), 4.96-5.00 (m, 1H), 7.05-7.15 (m, 3H), 7.42 (brs, 2H), 7.99 (s, 1H), 8.28 (s, 1H), 8.59-8.61 (m, 2H), 8.66 (s, 1H), 8.94 (d, J = 7.2 Hz, 1H), 10.83 (s, 1H). |
| P-171 | A54 | C59 | 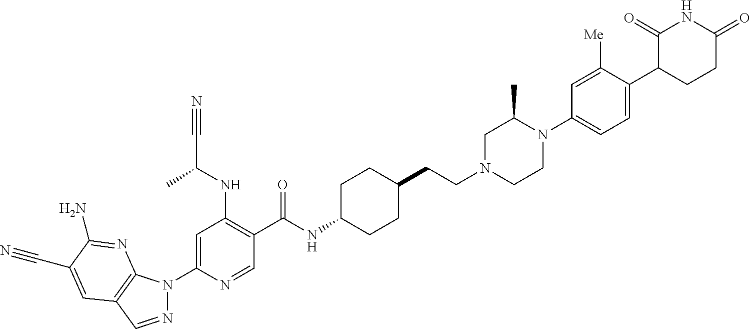 [SMILES: Cc1cc(N2CCN(CC[C@H]3CC[C@H](NC(=O)c4cnc(-n5ncc6cc(C#N)c(N)nc65)cc4N[C@H](C)C#N)CC3)C[C@H]2C)ccc1C1CCC(=O)NC1=O] | 9.0% yield as an off-white solid. LCMS method 8: Retention time: 1.668 min, 96.0% purity at 215 nm, [M + H]⁺ = 757.4 ¹H NMR (400 MHz, DMSO-d6, 80° C.): δ 0.96-1.12 (m, 1H), 1.12-1.15 (m, 4H), 1.32-1.44 (m, 3H), 1.64-1.72 (m, 6H), 1.84-1.87 (m, 2H), 1.95-2.02 (m, 3H), 2.10-2.15 (m, 1H), 2.27 (s, 3H), 3.54-3.68 (m, 2H), 3.75-3.85 (m, 1H), 3.90-3.98 (m, 1H), 4.28-4.32 (m, 1H), 4.92-4.96 (m, 1H), 6.80-7.05 (m, 5H), 7.82 (s, 1H), 8.21 (s, 1H), 8.31 (d, J = 7.6 Hz, 1H), 8.52 (s, 1H), 8.68 (s, 1H), 8.72 (d, J = 7.6 Hz, 1H), 10.50 (s, 1H). Seven protons were not apparent by ¹H NMR. |
| P-172 | A54 | C60 | 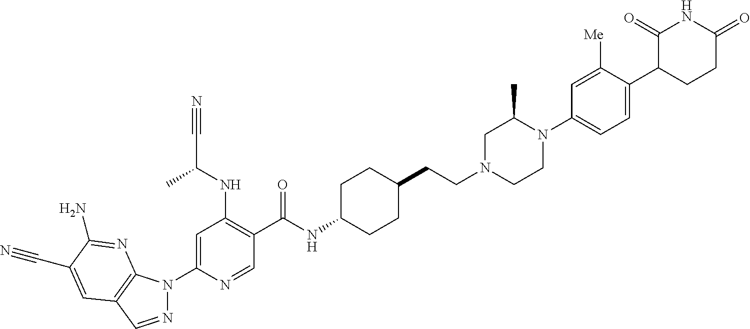 [SMILES: Cc1cc(N2CCN(CC[C@H]3CC[C@H](NC(=O)c4cnc(-n5ncc6cc(C#N)c(N)nc65)cc4N[C@H](C)C#N)CC3)C[C@H]2C)ccc1C1CCC(=O)NC1=O] | 28% yield as an off-white solid. LCMS method 7: Retention time: 1.066 min, 98.9% purity at 215 nm, [M + H]⁺ = 757.4 ¹H NMR (400 MHz, DMSO-d6): 8 1.08-1.17 (m, 5H), 1.29-1.42 (m, 3H), 1.58-1.64 (m, 2H), 1.71 (d, J = 6.8 Hz, 3H), 1.80-1.83 (m, 2H), 1.88-1.94 (m, 2H), 2.07-2.14 (m, 1H), 2.22-2.26 (m, 1H), 2.60-2.67 (m, 2H), 3.07-3.22 (m, 6H), 3.71-3.82 (m, 3H), 3.99-4.03 (m, 1H), 4.42-4.48 (m, 1H), 4.96-4.99 (m, 1H), 7.37-7.44 (m, 3H), 7.54 (d, J = 7.2 Hz, 1H), 8.01 (s, 1H), 8.28-8.33 (m, 2H), 8.59-8.62 (m, 2H), 8.66 (s, 1H), 8.95 (d, J = 7.2 Hz, 1H), 10.90 (s, 1H). |
| P-173 | A54 | C61 | 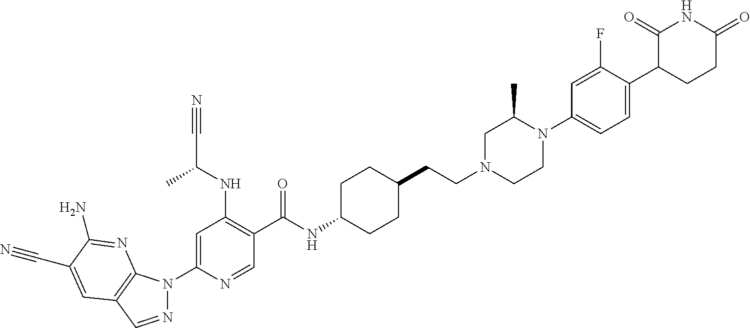 [SMILES: C[C@H](C#N)Nc1cc(-n2ncc3cc(C#N)c(N)nc32)ncc1C(=O)N[C@H]1CC[C@H](CCN2CCN(c3ccc(C4CCC(=O)NC4=O)c(F)c3)[C@H](C)C2)CC1] | 21% yield as an off-white solid. LCMS method 7: Retention time: 1.664 min, 97.0% purity at 215 nm, [M + H]⁺ = 761.2 ¹H NMR (400 MHz, DMSO-d6): δ ppm 1.03-1.09 (m, 4H), 1.33-1.41 (m, 5H), 1.69 (d, J = 6.8 Hz, 3H), 1.81-2.19 (m, 7H), 2.30-2.33 (m, 1H), 2.71-2.76 (m, 2H), 2.88-2.96 (m, 2H), 3.75-3.77 (m, 1H), 3.87 (dd, J = 4.4 Hz and 12.4 Hz, 1H), 4.01-4.03 (m, 1H), 4.92-4.96 (m, 1H), 6.66 (d, J = 11.20 Hz, 2H), 7.07 (t, J = 8.80 Hz, 1H), 7.35 (brs, 2H), 7.89 (s, 1H), 8.23 (s, 1H), 8.49 (d, J = 8.00 Hz, 1H), 8.57 (s, 1H), ), 8.66 (s, 1H), 8.79 (d, J = 7.20 Hz, 1H), 10.81 (s, 1H) Seven protons were not apparent by ¹H NMR. |
| P-177 | A54 | C62 | 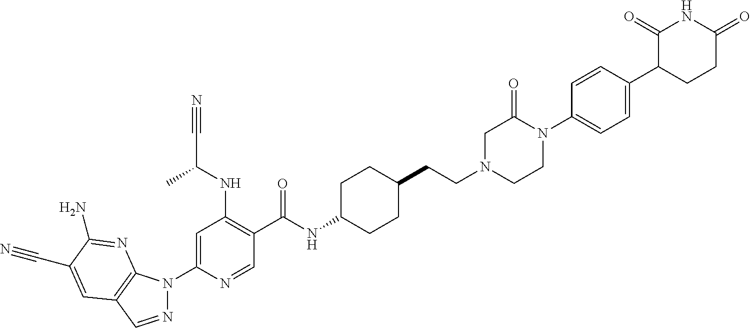 | 26% yield as an off-white solid. LCMS method 8: Retention time: 1.695 min, 98.5% purity at 215 nm, [M + H]⁺ = 743.1 ¹H-NMR (400 MHz, DMSO-d6): 8 1.05-1.15 (m, 2H), 1.35-1.43 (m, 3H), 1.62-1.68 (m, 2H), 1.71 (d, J = 8.4 Hz, 3H), 1.82-1.85 (m, 2H), 1.89-1.95 (m, 2H), 2.03-2.09 (m, 1H), 2.19-2.28 (m, 1H), 3.22-3.28 (m, 2H), 3.73-3.79 (m, 2H), 3.89-3.96 (m, 4H), 4.94-4.97 (m, 1H), 7.23 (m, 4H), 7.35 (brs, 2H), 7.93 (s, 1H), 8.24 (s, 1H), 8.53 (d, J = 8.0 Hz, 1H), 8.57 (s, 1H), 8.67 (s, 1H), 8.81 (d, J = 7.2 Hz, 1H), 10.87 (s, 1H). Four protons were not apparent by ¹H NMR. |
| P-178 | A54 | C44 | 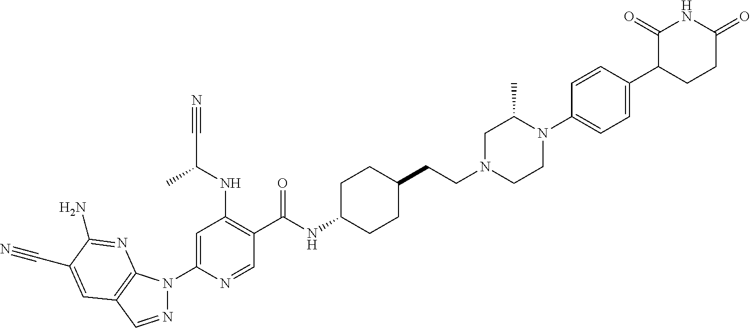 [SMILES: C[C@H](C#N)Nc1cc(-n2ncc3cc(C#N)c(N)nc32)ncc1C(=O)N[C@H]1CC[C@H](CCN2CCN(c3ccc(C4CCC(=O)NC4=O)cc3)[C@@H](C)C2)CC1] | 30% yield as an off-white solid. LCMS method 8: Retention time: 1.784 min, 95.8% purity at 215 nm, [M + H]⁺ = 743.1 ¹H NMR (400 MHz, DMSO-d6): δ ppm 0.92 (d, J = 6.0 Hz, 1H), 1.08-1.18 (m, 4H), 1.34-1.43 (m, 3H), 1.60-1.69 (m, 2H), 1.71 (d, J = 6.8 Hz, 3H), 1.81-1.84 (m, 2H), 1.89-2.04 (m, 3H), 2.09-2.21 (m, 1H), 2.60-2.65 (m, 1H), 2.80-2.86 (m, 1H), 3.06-3.29 (m, 6H), 3.56-3.64 (m, 4H), 4.32-4.38 (m, 1H), 4.95-4.99 (m, 1H), 6.92 and 7.21 (d, J = 8.40 Hz, 1.4H and 0.6H), 7.10-7.13 (m, 2H), 7.39 (brs, 2H), 7.97 (s, 1H), 8.27 (s, 1H), 8.56 (d, J = 8.0 Hz, 1H), 8.59 (s, 1H), 8.67 (s, 1H), 8.88 (d, J = 7.60 Hz, 1H), 10.79 (s, 1H). |
| P-179 | A29 | C63 | 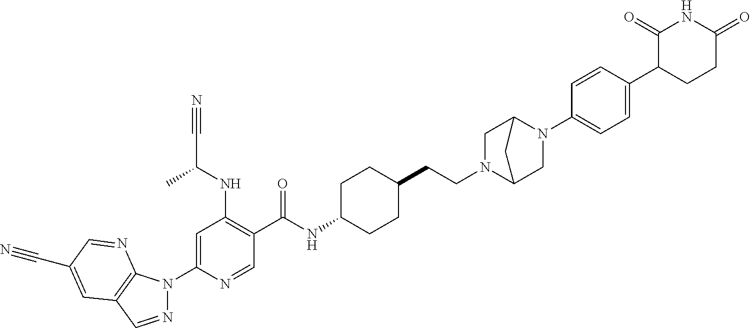 [SMILES: C[C@H](C#N)Nc1cc(-n2ncc3cc(C#N)cnc32)ncc1C(=O)N[C@H]1CC[C@H](CCN2CC3CC2CN3c2ccc(C3CCC(=O)NC3=O)cc2)CC1] | 8.9% yield as an off-white solid. LCMS method 7: Retention time: 1.313 min, 93.2% purity at 215 nm, [M + H]⁺ = 726.2 ¹H NMR (400 MHz, DMSO-d6): δ ppm 0.96-1.05 (m, 2H), 1.22-1.34 (m, 6H), 1.66 (d, J = 6.8 Hz, 3H), 1.72-1.75 (m, 2H), 1.83-1.89 (m, 2H), 1.98-2.12 (m, 2H), 2.82-2.84 (m, 1H), 3.10-3.13 (m, 1H), 3.28-3.30 (m, 2H), 4.24 (brs, 2H), 4.89-4.93 (m, 1H), 6.51 (d, J = 8.4 Hz, 2H), 6.98 (d, J = 8.4 Hz, 2H), 7.48 (s, 1H), 8.58 (d, J = 8.0 Hz, 1H), 8.68 (s, 1H), 8.69 (s, 1H), 8.75 (d, J = 7.2 Hz, 1H), 9.01-9.04 (m, 2H), 10.75 (s, 1H). Seven protons were not apparent by ¹H NMR. |
| P-180 | A29 | C64 | 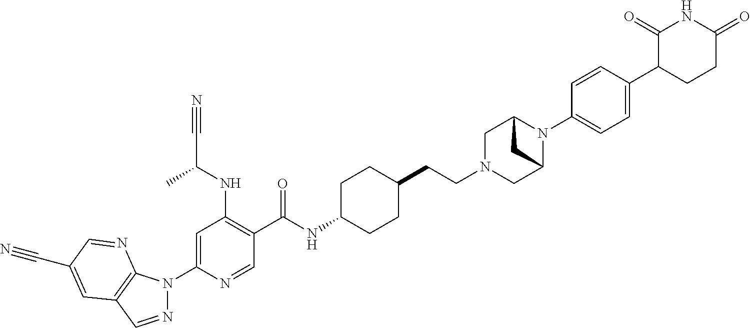 [SMILES: C[C@H](C#N)Nc1cc(-n2ncc3cc(C#N)cnc32)ncc1C(=O)N[C@H]1CC[C@H](CCN2C[C@H]3C[C@@H](C2)N3c2ccc(C3CCC(=O)NC3=O)cc2)CC1] | 24% yield as a brown solid. LCMS method 7: Retention time: 1.421 min, 96.0% purity at 215 nm, [M + H]⁺ = 726.4 ¹H NMR (400 MHz, DMSO-d6): δ ppm 0.94-1.01 (m, 2H), 1.15-1.35 (m, 5H), 1.67 (d, J = 6.80 Hz, 3H), 1.82-1.91 (m, 2H), 2.02-2.21 (m, 3H), 2.60-2.66 (m, 1H), 2.71-2.76 (m, 1H), 2.82-2.86 (m, 2H), 3.70-3.79 (m, 4H), 4.39 (d, J = 6.00 Hz, 2H), 4.93-4.98 (m, 1H), 6.59 (d, J = 8.40 Hz, 2H), 7.15 (d, J = 8.40 Hz, 2H), 7.49 (s, 1H), 8.52 (d, J = 8.0 Hz, 1H), 8.69 (s, 1H), 8.70 (d, J = 4.4 Hz, 1H), 8.75 (d, J = 7.2 Hz, 1H), 9.04 (s, 2H), 10.80 (s, 1H). Five protons were not apparent by ¹H NMR. |
| P-181 | A54 | C65 | 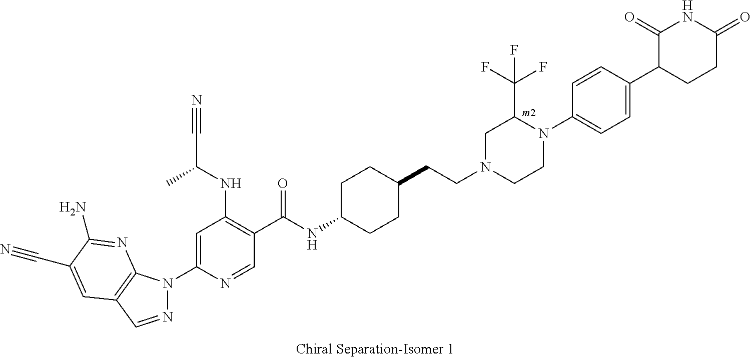 [SMILES: C[C@H](C#N)Nc1cc(-n2ncc3cc(C#N)c(N)nc32)ncc1C(=O)N[C@H]1CC[C@H](CCN2CCN(c3ccc(C4CCC(=O)NC4=O)cc3)C(C(F)(F)F)C2)CC1] | 30% yield as an off-white solid. LCMS method 7: Retention time: 1.731 min, 96.7% purity at 215 nm, [M + H]⁺ = 797.2 ¹H NMR (400 MHz, DMSO-d6): δ ppm 1.05-1.13 (m, 2H), 1.23-1.43 (m, 4H), 1.55-1.60 (m, 1H), 1.70 (d, J = 6.8 Hz, 3H), 1.79-1.83 (m, 2H), 1.89-2.04 (m, 3H), 2.09-2.19 (m, 1H), 2.61-2.65 (m, 1H), 3.10-3.21 (m, 2H), 3.42-3.52 (m, 4H), 4.95-4.98 (m, 1H), 5.22-5.28 (m, 1H), 7.03 (d, J = 7.6 Hz, 2H), 7.13 (d, J = 7.6 Hz, 2H), 7.39 (brs, 2H), 7.96 (s, 1H), 8.26 (s, 1H), 8.54 (d, J = 7.6 Hz, 1H), 8.58 (s, 1H), 8.66 (s, 1H), 8.87 (brs, 1H), 10.79 (s, 1H). ¹⁹NMR (400 MHz, DMSO-d6): δ ppm −66.27. Five protons were not apparent by ¹H NMR. |
| P-182 | A29 | C66 | 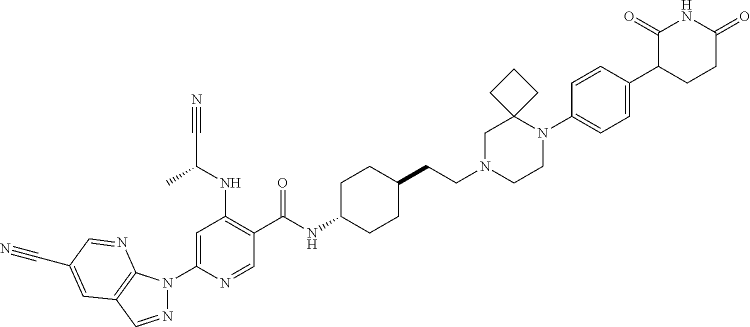 [SMILES: C[C@H](C#N)Nc1cc(-n2ncc3cc(C#N)cnc32)ncc1C(=O)N[C@H]1CC[C@H](CCN2CCN(c3ccc(C4CCC(=O)NC4=O)cc3)C3(CCC3)C2)CC1] | 59% yield as an off-white solid. LCMS method 7: Retention time: 1.736 min, 94.7% purity at 215 nm, [M + H]⁺ = 754.4 ¹H NMR (400 MHz, DMSO-d6): δ ppm 1.05-1.15 (m, 2H), 1.26-1.51 (m, 4H), 1.61-1.83 (m, 8H), 1.91-2.06 (m, 5H), 2.14-2.18 (m, 2H), 2.89-2.96 (m, 2H), 3.15-3.23 (m, 5H), 3.80-3.84 (m, 3H), 4.93-4.98 (m, 1H), 6.92 (d, J = 8.40 Hz, 2H), 7.16 (d, J = 8.80 Hz, 2H), 7.50 (s, 1H), 8.58 (d, J = 7.60 Hz, 1H), 8.70 (s, 1H), 8.72 (s, 1H), 8.76 (d, J = 7.60 Hz, 1H), 9.03-9.05 (m, 2H), 10.83 (s, 1H). Three protons were not apparent by ¹H NMR. |
| P-183 | A29 | C67 | 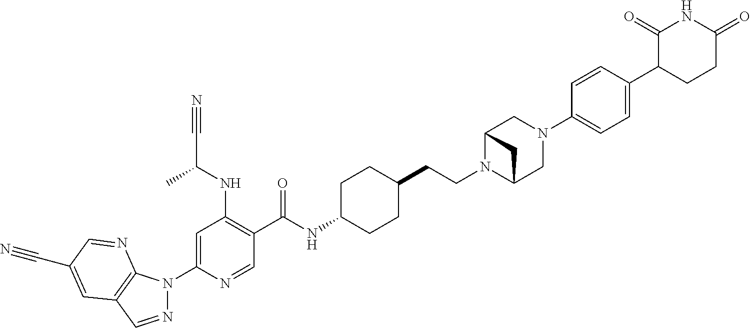 [SMILES: C[C@H](C#N)Nc1cc(-n2ncc3cc(C#N)cnc32)ncc1C(=O)N[C@H]1CC[C@H](CCN2[C@@H]3C[C@H]2CN(c2ccc(C4CCC(=O)NC4=O)cc2)C3)CC1] | 18% yield as an off-white solid. LCMS method 7: Retention time: 1.864 min, 97.5% purity at 215 nm, [M + H]⁺ = 726.1 ¹H NMR (400 MHz, DMSO-d6): δ ppm 0.98-1.12 (m, 2H), 1.23-1.55 (m, 6H), 1.65-1.68 (m, 3H), 1.74-2.03 (m, 7H), 2.09-2.21 (m, 1H), 2.58-2.62 (m, 1H), 2.81-2.90 (m, 2H), 3.30-3.33 (m, 1H), 3.42-3.48 (m, 2H), 4.44-4.46 (m, 1H), 4.60-4.62 (m, 1H), 4.93-4.98 (m, 1H), 6.75 (d, J = 8.8 Hz, 2H) 7.13 (d, J = 8.8 Hz, 2H), 7.49 (d, J = 5.2 Hz, 1H), 8.54-8.59 (m, 1H), 8.69-8.79 (m, 3H), 9.05 (s, 1H), 9.06 (s, 1H), 10.79 (s, 1H). Three protons were not apparent by ¹H NMR. |
| P-184 | A73 | C12 | 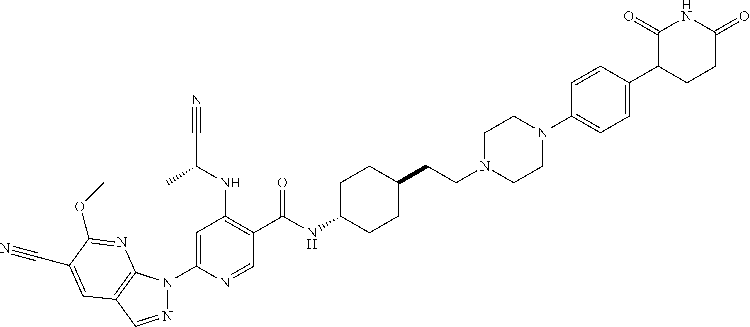 [SMILES: COc1nc2c(cnn2-c2cc(N[C@H](C)C#N)c(C(=O)N[C@H]3CC[C@H](CCN4CCN(c5ccc(C6CCC(=O)NC6=O)cc5)CC4)CC3)cn2)cc1C#N] | 19% yield as an off-white solid. LCMS method 7: Retention time: 1.420 min, 95.8% purity at 215 nm, [M + H]⁺ = 744.2 ¹H NMR (400 MHz, DMSO-d6): 8 1.05-1.15 (m, 2H), 1.31-1.41 (m, 4H), 1.60-1.66 (m, 2H), 1.67 (d, J = 7.2 Hz, 3H), 1.80-1.85 (m, 2H), 1.90-2.04 (m, 3H), 2.10-2.20 (m, 1H), 2.56-2.67 (m, 1H), 2.95-3.01 (m, 2H), 3.12-3.30 (m, 4H), 3.58-3.61 (m, 2H), 3.74-3.85 (m, 4H), 4.18 (s, 3H), 4.98-5.00 (m, 1H), 6.98 (d, J = 8.8 Hz, 2H), 7.12 (d, J = 8.8 Hz, 2H), 7.64 (s, 1H), 8.50 (s, 1H), 8.59 (d, J = 7.6 Hz, 1H), 8.73 (s, 1H), 8.88 (d, J = 7.2 Hz, 1H), 8.92 (s, 1H), 10.80 (s, 1H). |
| P-185 | A54 | C12 | 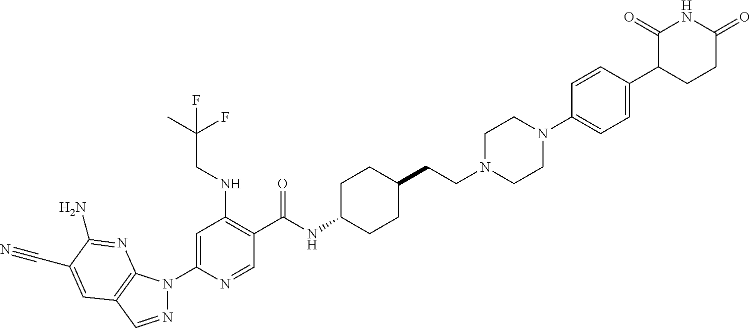 [SMILES: CC(F)(F)CNc1cc(-n2ncc3cc(C#N)c(N)nc32)ncc1C(=O)N[C@H]1CC[C@H](CCN2CCN(c3ccc(C4CCC(=O)NC4=O)cc3)CC2)CC1] | 5.6% yield as an off-white solid. LCMS method 7: Retention time: 1.336 min, 95.1% purity at 215 nm, [M + H]⁺ = 754.2 ¹H NMR (400 MHz, DMSO-d6): 8 0.98-1.08 (m, 2H), 1.24-1.42 (m, 6H), 1.70 (t, J = 18.8 Hz, 3H), 1.80-1.89 (m, 4H), 1.99-2.03 (m, 1H), 2.12-2.18 (m, 1H), 2.34-2.41 (m, 2H), 2.58-2.64 (m, 1H), 3.12-3.18 (m, 4H), 3.71-3.88 (m, 4H), 6.89 (d, J = 8.8 Hz, 2H), 7.05 (d, J = 8.8 Hz, 2H), 7.30 (brs, 2H), 7.54 (s, 1H), 8.18 (s, 1H), 8.41 (d, J = 8.0 Hz, 1H), 8.54 (s, 1H), 8.58 (s, 1H), 8.87 (t, J = 6.4 Hz, 1H), 10.84 (s, 1H). Four protons were not apparent by ¹H NMR. |
| P-186 | A54 | C65 | 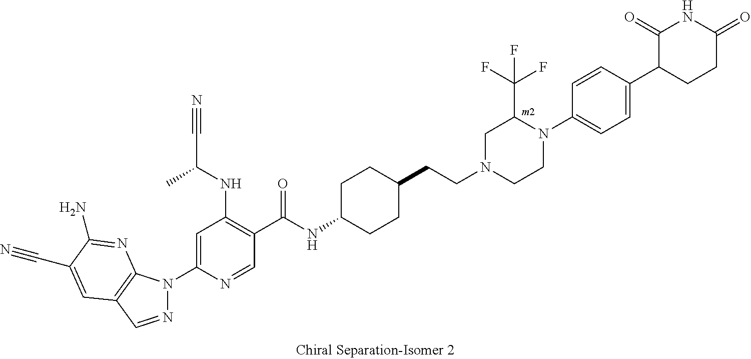 [SMILES: C[C@H](C#N)Nc1cc(-n2ncc3cc(C#N)c(N)nc32)ncc1C(=O)N[C@H]1CC[C@H](CCN2CCN(c3ccc(C4CCC(=O)NC4=O)cc3)C(C(F)(F)F)C2)CC1] | 21% yield as an off-white solid. LCMS method 7: Retention time: 1.553 min, 99.5% purity at 215 nm, [M + H]⁺ = 797.2 ¹H NMR (400 MHz, DMSO-d6): 8 1.07-1.12 (m, 2H), 1.24-1.42 (m, 4H), 1.51-1.65 (m, 2H), 1.70 (d, J = 7.3 Hz, 3H), 1.71-1.75 (m, 2H), 1.78-1.84 (m, 2H), 1.91-2.03 (m, 1H), 2.12-2.18 (m, 1H), 2.60-2.67 (m, 1H), 3.71-3.78 (m, 4H), 4.94-4.98 (m, 1H), 5.22-5.28 (m, 1H), 7.03 (d, J = 7.6 Hz, 2H), 7.12 (d, J = 7.6 Hz, 2H), 7.39 (brs, 2H), 7.95 (s, 1H), 8.26 (s, 1H), 8.54 (d, J = 7.6 Hz, 1H), 8.58 (s, 1H), 8.66 (s, 1H), 8.87 (d, J = 6.4 Hz, 1H), 10.80 (s, 1H). ¹⁹NMR (400 MHz, DMSO-d6): δ ppm −66.27. Six protons were not apparent by ¹H NMR. |
| P-187 | A54 | C68 | 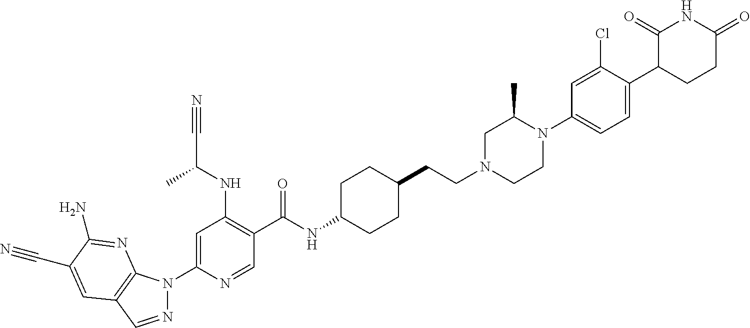 | 31% yield as an off-white solid. LCMS method 7: Retention time: 1.882 min, 94.9% purity at 215 nm, [M + H]⁺ = 777.2 ¹H NMR (400 MHz, DMSO-d6): δ ppm 0.97-1.15 (m, 5H), 1.23-1.43 (m, 3H), 1.59-1.69 (m, 2H), 1.70 (d, J = 6.8 Hz, 3H), 1.80-1.84 (m, 2H), 1.89-1.95 (m, 3H), 2.21-2.26 (m, 1H), 2.71-2.80 (m, 1H), 3.07-3.23 (m, 5H), 3.44-3.58 (m, 3H), 4.40-4.42 (m, 1H), 4.95-4.97 (m, 1H), 6.91-7.01 (m, 2H), 7.17-7.20 (m, 1H), 7.41 (bs, 2H), 7.98 (d, J = 4.8 Hz, 1H), 8.27 (s, 1H), 8.56 (d, J = 8.0 Hz, 1H), 8.59 (s, 1H), 8.66 (d, J = 4.4 Hz, 1H), 8.88 (d, J = 6.4 Hz, 1H), 10.86 (s, 1H) Three protons were not apparent by ¹H NMR. |
| P-188 | A74 | C12 | 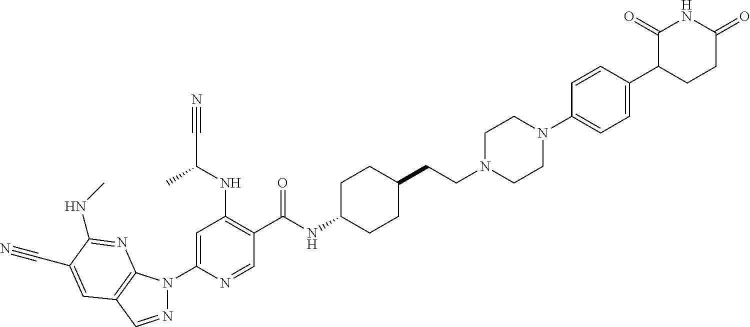 [SMILES: CNc1nc2c(cnn2-c2cc(N[C@H](C)C#N)c(C(=O)N[C@H]3CC[C@H](CCN4CCN(c5ccc(C6CCC(=O)NC6=O)cc5)CC4)CC3)cn2)cc1C#N] | 27% yield as an off-white solid. LCMS method 7: Retention time: 1.832 min, 97.7% purity at 215 nm, [M + H]⁺ = 743.4 ¹H NMR (400 MHz, DMSO-d6): 8 1.03-1.12 (m, 2H), 1.33-1.40 (m, 3H), 1.60-1.61 (m, 2H), 1.68 (d, J = 7.2 Hz, 3H), 1.80-1.83 (m, 2H), 1.88-1.92 (m, 2H), 1.99-2.02 (m, 1H), 2.14-2.16 (m, 1H), 2.65-2.68 (m, 1H), 2.95-3.03 (m, 5H), 3.12-3.21 (m, 4H), 3.75-3.84 (m, 4H), 4.89-4.91 (m, 1H), 6.98 (d, J = 8.8 Hz, 2H), 7.12 (d, J = 8.4 Hz, 2H), 7.67 (brs, 1H), 7.91 (s, 1H), 8.26 (s, 1H), 8.56 (s, 1H), 8.60 (d, J = 8.0 Hz, 1H), 8.68 (s, 1H), 8.95 (d, J = 7.2 Hz, 1H), 10.80 (s, 1H) Three protons were not apparent by ¹H NMR. |
| P-189 | A29 | C69 | 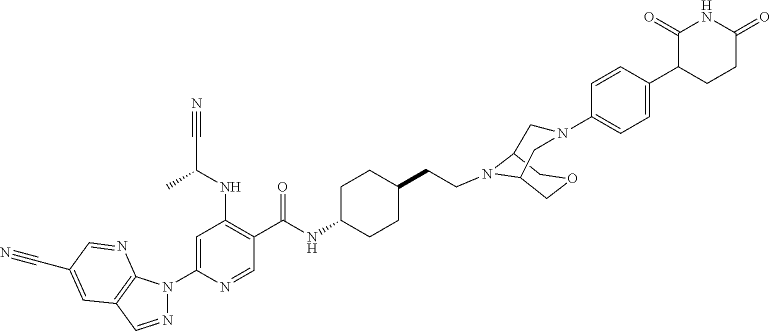 [SMILES: C[C@H](C#N)Nc1cc(-n2ncc3cc(C#N)cnc32)ncc1C(=O)N[C@H]1CC[C@H](CCN2C3COCC2CN(c2ccc(C4CCC(=O)NC4=O)cc2)C3)CC1] | 17% yield as an off-white solid. LCMS method 8: Retention time: 1.810 min, 96.7% purity at 215 nm, [M + H]⁺ = 756.4 ¹H NMR (400 MHz, DMSO-d6): δ ppm 1.06-1.18 (m, 2H), 1.31-1.40 (m, 4H), 1.61-1.68 (m, 5H), 1.82-2.03 (m, 6H), 2.12-2.20 (m, 1H), 3.71-3.80 (m, 5H), 3.87-3.95 (m, 2H), 4.04-4.18 (m, 4H), 4.38-4.42 (m, 1H), 4.93-4.97 (m, 1H), 4.94-4.97 (m, 1H), 6.86 (d, J = 8.40 Hz, 2H), 7.12 (d, J = 8.40 Hz, 2H), 7.49 (s, 1H), 8.55 (d, J = 7.60 Hz, 1H), 8.70 (s, 1H), 8.71 (s, 1H), 8.77 (d, J = 7.20 Hz, 1H), 9.05 (s, 2H), 10.79 (s, 1H) One proton was not apparent by ¹H NMR. |
| P-190 | A29 | C70 | 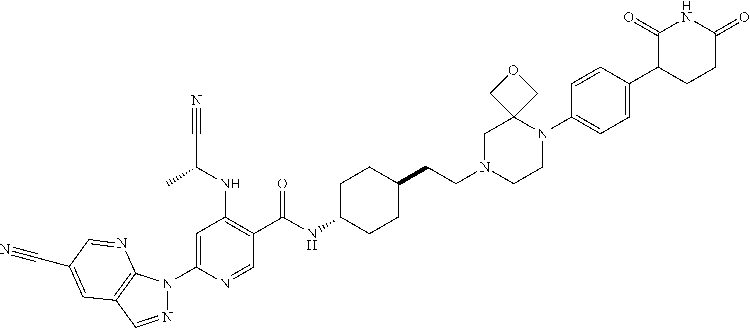 [SMILES: C[C@H](C#N)Nc1cc(-n2ncc3cc(C#N)cnc32)ncc1C(=O)N[C@H]1CC[C@H](CCN2CCN(c3ccc(C4CCC(=O)NC4=O)cc3)C3(COC3)C2)CC1] | 18% yield as an off-white solid. LCMS method 8: Retention time: 1.807 min, 99.0% purity at 215 nm, [M + H]⁺ = 756.8 ¹H NMR (400 MHz, DMSO-d6) δ ppm: 1.04-1.13 (m, 2H), 1.25-1.42 (m, 3H), 1.61-1.66 (m, 2H), 1.67 (d, J = 7.2 Hz, 3H), 1.79-1.82 (m, 2H), 1.89-2.05 (m, 3H), 2.12-2.22 (m, 1H), 2.78-2.89 (m, 1H), 3.22-3.31 (m, 4H), 3.39-3.45 (m, 2H), 3.80-3.84 (m, 2H), 4.09-4.12 (m, 1H), 4.51-4.60 (m, 3H), 4.73-4.75 (m, 1H), 4.92-4.99 (m, 1H), 6.79 (d, J = 8.8 Hz, 2H), 7.16 (d, J = 8.8 Hz, 2H), 7.50 (s, 1H), 8.57 (d, J = 8.0 Hz, 1H), 8.70 (s, 1H), 8.72 (s, 1H), 8.76 (d, J = 7.2 Hz, 1H), 9.04-9.05 (m, 2H), 10.82 (s, 1H) Two protons were not apparent by ¹H NMR. |
| P-191 | A29 | C70 | 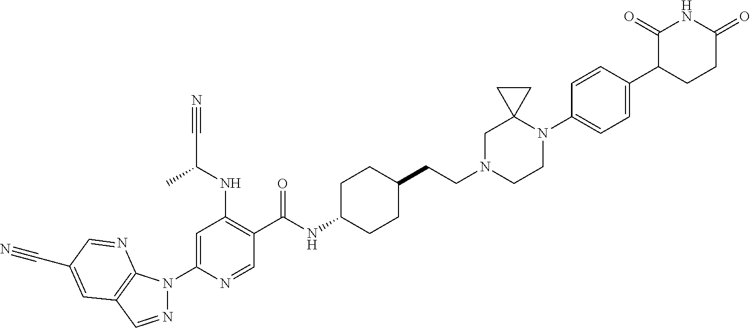 [SMILES: C[C@H](C#N)Nc1cc(-n2ncc3cc(C#N)cnc32)ncc1C(=O)N[C@H]1CC[C@H](CCN2CCN(c3ccc(C4CCC(=O)NC4=O)cc3)C3(CC3)C2)CC1] | 23% yield as an off-white solid. LCMS method 8: Retention time: 1.872 min, 99.5% purity at 215 nm, [M + H]⁺ = 740.3 ¹H NMR (400 MHz, DMSO-d6): δ ppm 0.80-0.85 (m, 1H), 0.96-1.11 (m, 4H), 1.24-1.41 (m, 4H), 1.53-1.58 (m, 2H), 1.67 (d, J = 7.2 Hz, 3H), 1.75-1.78 (m, 2H), 1.88-2.04 (m, 3H), 2.12-2.20 (m, 1H), 2.63-2.67 (m, 1H), 2.81-2.90 (m, 1H), 2.95-3.00 (m, 1H), 3.12-3.16 (m, 2H), 3.32-3.38 (m, 1H), 3.46-3.61 (m, 2H), 4.11-4.16 (m, 1H), 4.91-4.98 (m, 1H), 7.04 (d, J = 8.80 Hz, 2H), 7.13 (d, J = 8.80 Hz, 2H), 7.49 (s, 1H), 8.55 (d, J = 7.60 Hz, 1H), 8.70 (s, 1H), 8.71 (s, 1H), 8.75 (d, J = 7.2 Hz, 1H), 9.04-9.05 (m, 2H), 10.80 (s, 1H) Three protons were not apparent by ¹H NMR. |
| P-192 | A75 | C12 | 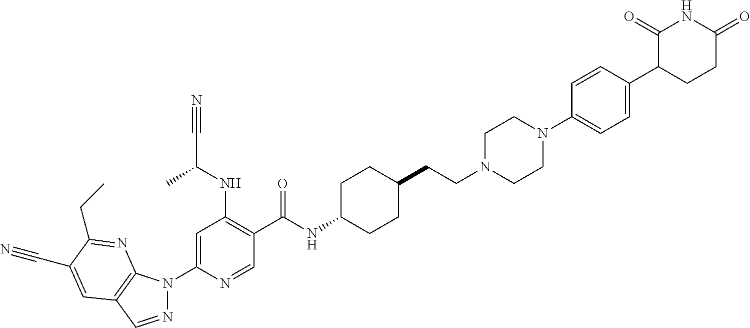 [SMILES: CCc1nc2c(cnn2-c2cc(N[C@H](C)C#N)c(C(=O)N[C@H]3CC[C@H](CCN4CCN(c5ccc(C6CCC(=O)NC6=O)cc5)CC4)CC3)cn2)cc1C#N] | 27% yield as an off-white solid. LCMS method 8: Retention time: 1.937 min, 92.8% purity at 215 nm, [M + H]⁺ = 742.3 ¹H NMR (400 MHz, DMSO-d6): δ ppm 1.06-1.15 (m, 2H), 1.30-1.43 (m, 7H), 1.61-1.64 (m, 2H), 1.70 (d, J = 7.2 Hz, 2H), 1.81-1.84 (m, 2H), 1.91-2.03 (m, 3H), 2.09-2.14 (m, 1H), 2.62-2.68 (m, 1H), 2.94-2.99 (m, 2H), 3.13-3.24 (m, 6H), 3.59-3.62 (m, 2H), 3.75-3.86 (m, 4H), 4.91-4.98 (m, 1H), 6.99 (d, J = 8.80 Hz, 2H), 7.12 (d, J = 8.80 Hz, 2H), 7.79 (s, 1H), 8.58-8.63 (m, 2H), 8.74 (s, 1H), 8.86 (d, J = 7.20 Hz, 1H), 8.98 (s, 1H), 10.81 (s, 1H). One proton was not apparent by ¹H NMR. |
| P-193 | A76 | C12 | 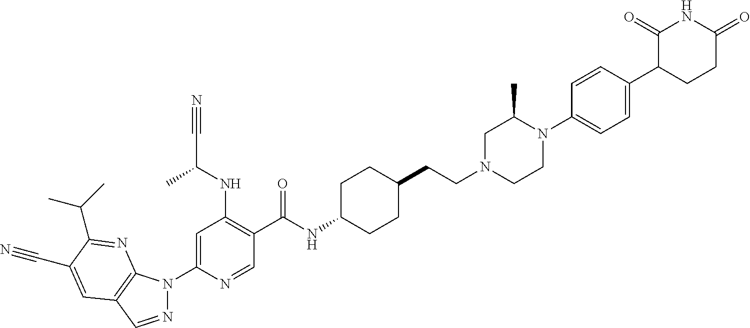 | 11% yield as an off-white solid. LCMS method 8: Retention time: 1.993 min, 99.7% purity at 215 nm, [M + H]⁺ = 756.4 ¹H NMR (400 MHz, DMSO-d6): δ ppm 1.04-1.09 (m, 2H), 1.22-1.42 (m, 12H), 1.70 (d, J = 6.80 Hz, 3H), 1.80-1.83 (m, 2H), 1.89-1.94 (m, 2H), 1.96-2.14 (m, 2H), 2.59-2.68 (m, 4H), 3.07-3.21 (m, 4H), 3.58-3.62 (m, 1H), 3.71-3.76 (m, 2H), 4.85-4.90 (m, 1H), 6.90 (d, J = 8.80 Hz, 2H), 7.06 (d, J = 8.80 Hz, 2H), 7.78 (s, 1H), 8.56 (d, J = 7.6 Hz, 1H), 8.61 (s, 1H), 8.74 (s, 1H), 8.91 (d, J = 7.20 Hz, 1H), 8.97 (s, 1H), 10.78 (s, 1H). Three protons were not apparent by ¹H NMR. |
| P-194 | A29 | C72 | 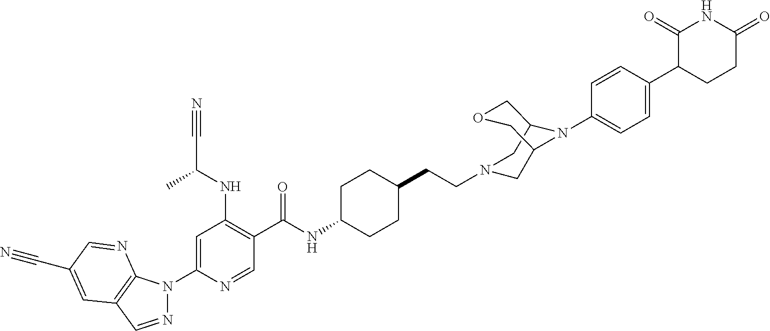 [SMILES: C[C@H](C#N)Nc1cc(-n2ncc3cc(C#N)cnc32)ncc1C(=O)N[C@H]1CC[C@H](CCN2CC3COCC(C2)N3c2ccc(C3CCC(=O)NC3=O)cc2)CC1] | 44% yield as an off-white solid. LCMS method 7: Retention time: 1.265 min, 99.3% purity at 215 nm, [M + H]⁺ = 756.3 ¹H NMR (400 MHz, DMSO-d6): δ ppm 1.03-1.11 (m, 2H), 1.22-1.28 (m, 1H), 1.31-1.39 (m, 2H), 1.62-1.65 (m, 2H), 1.67 (d, J = 7.2 Hz, 3H), 1.76-1.79 (m, 2H), 1.88-1.93 (m, 2H), 1.99-2.04 (m, 1H), 2.12-2.18 (m, 1H), 2.61-2.65 (m, 1H), 3.04-3.20 (m, 4H), 3.67-3.78 (m, 4H), 3.84-3.87 (m, 2H), 4.09-4.12 (m, 2H), 4.22-4.23 (m, 2H), 4.91-4.98 (m, 1H), 6.94 (d, J = 8.40 Hz, 2H), 7.14 (d, J = 8.80 Hz, 2H), 7.49 (s, 1H), 8.57 (d, J = 7.60 Hz, 1H), 8.70 (s, 1H), 8.71 (s, 1H), 8.76 (d, J = 7.2 Hz, 1H), 9.04-9.05 (m, 2H), 10.80 (s, 1H). One proton was not apparent by ¹H NMR. |
| P-197 | A79 | C12 | 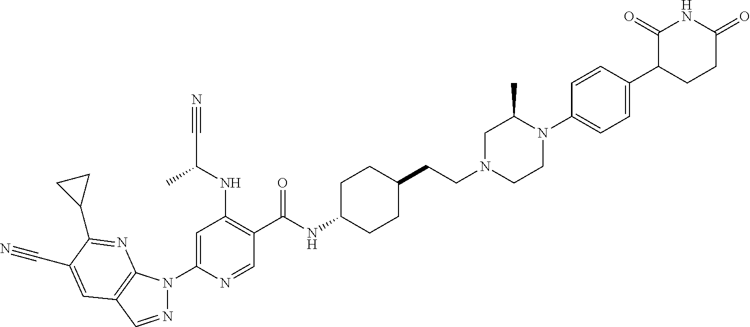 | 48% yield as a pale-yellow solid. LCMS method 7: Retention time: 1.595 min, 98.2% purity at 215 nm, [M + H]⁺ = 754.3 ¹H NMR (400 MHz, DMSO-d6): 8 1.08-1.13 (m, 2H), 1.26-1.40 (m, 8H), 1.61-1.65 (m, 2H), 1.70 (d, J = 7.20 Hz, 3H), 1.81-1.84 (m, 2H), 1.92-2.03 (m, 3H), 2.11-2.17 (m, 1H), 2.61-2.68 (m, 2H), 2.94-3.00 (m, 2H), 3.11-3.23 (m, 4H), 3.76-3.86 (m, 4H), 4.92-4.95 (m, 1H), 6.98 (d, J = 8.80 Hz, 2H), 7.12 (d, J = 8.80 Hz, 2H), 7.63 (s, 1H), 8.57-8.59 (m, 2H), 8.73 (s, 1H), 8.89-8.92 (m, 2H), 10.80 (s, 1H) Two protons were not apparent by ¹H NMR. |
| P-200 | A54 | C12 | 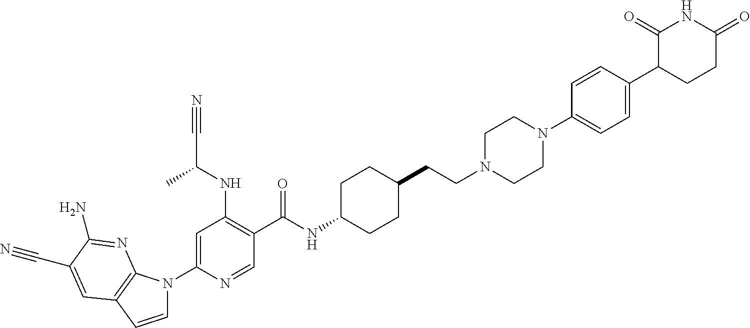 [SMILES: C[C@H](C#N)Nc1cc(-n2ccc3cc(C#N)c(N)nc32)ncc1C(=O)N[C@H]1CC[C@H](CCN2CCN(c3ccc(C4CCC(=O)NC4=O)cc3)CC2)CC1] | 9.3% yield as an off-white solid. LCMS method 7: Retention time: 1.980 min, 97.5% purity at 215 nm, [M + H]⁺ = 728.3 ¹H NMR (400 MHz, DMSO-d6): δ ppm 1.05-1.13 (m, 2H), 1.28-1.45 (m, 4H), 1.60-1.63 (m, 2H), 1.72 (d, J = 6.8 Hz, 3H), 1.81-1.93 (m, 4H), 1.95-2.02 (m, 1H), 2.13-2.19 (m, 2H), 2.92-2.99 (m, 2H), 3.14-3.23 (m, 4H), 3.56-3.62 (m, 2H), 3.75-3.85 (m, 4H), 4.97-5.01 (m, 1H), 6.59 (d, J = 4.0 Hz, 1H), 6.87 (brs, 2H), 6.98 (d, J = 8.4 Hz, 2H), 7.12 (d, J = 8.8 Hz, 2H), 8.14 (d, J = 4.0 Hz, 1H), 8.25 (s, 1H), 8.45 (d, J = 7.6 Hz, 1H), 8.47 (s, 1H), 8.63 (s, 1H), 8.80 (d, J = 7.2 Hz, 1H), 10.79 (s, 1H). |

General Procedure X-9. The scheme shown below for the synthesis of P-54 is provided as a representative synthesis for General Procedure X-9.

### Example S48. 1-(4-(Cyclopentylamino)-5-(1-((1r,4r)-4-(2-(4-(4-(2,6-dioxopiperidin-3-yl)phenyl)piperazin-1-yl)ethyl)cyclohexyl)-1H-1,2,3-triazol-4-yl)pyridin-2-yl)-1H-pyrazolo[3,4-b]pyridine-5-carbonitrile (P-54)

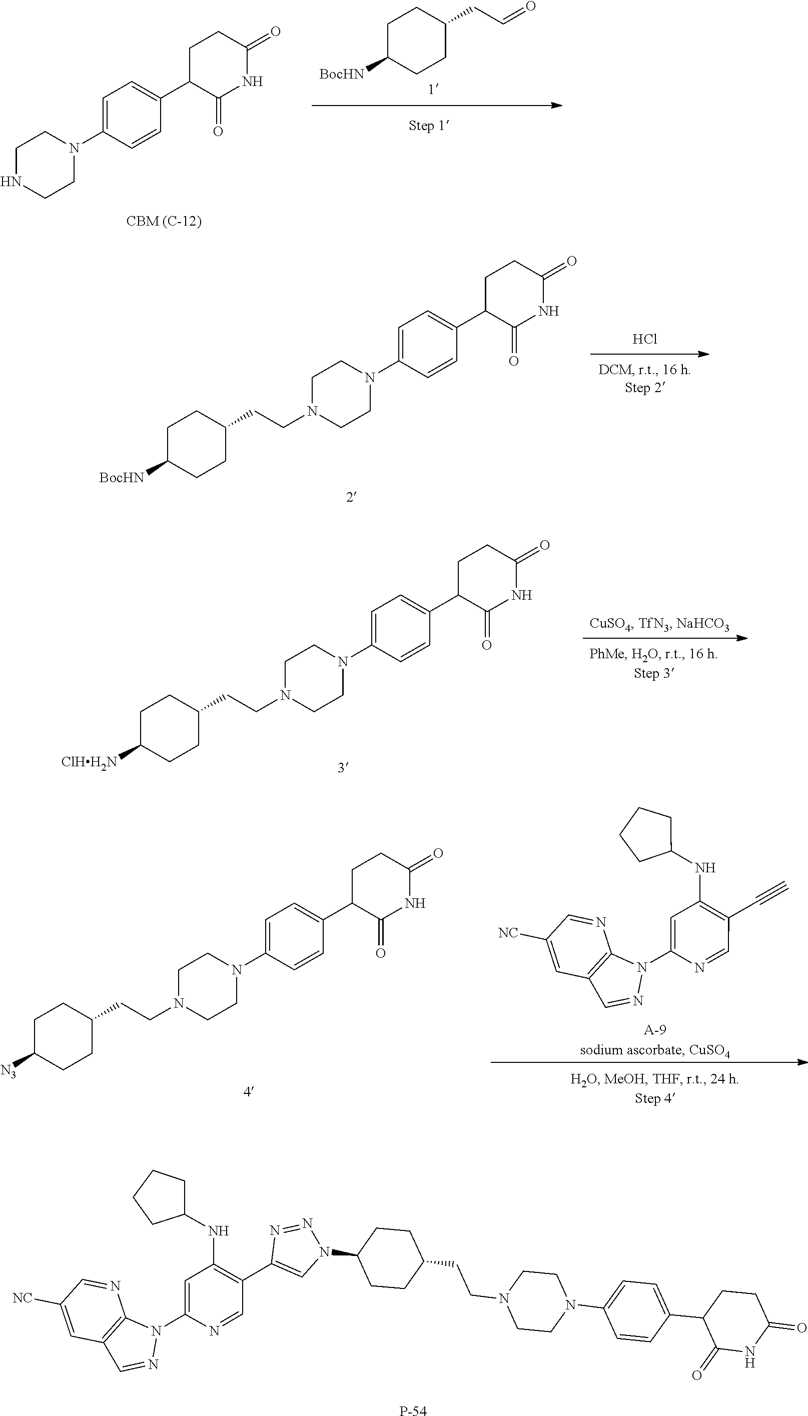

Step 1. Preparation of tert-butyl (4-(2-(4-(4-(2,6-dioxopiperidin-3-yl)phenyl)piperazin-1-yl)ethyl)cyclohexyl)carbamate (2′): 3-(4-piperazin-1-ylphenyl)piperidine-2,6-dione;sulfuric acid C-12 (274. mg, 0.7400 mmol) was dissolved with triethylamine (1.23 mL, 8.85 mmol) in DCM (5.5771 mL), then tert-butyl N-[4-(2-oxoethyl)cyclohexyl]carbamate 1 (195.84 mg, 0.8100 mmol) was added to the mixture. After 5 minutes of stirring, NaBH(OAc)₃ (469.06 mg, 2.21 mmol) was added to the mixture. LCMS showed complete conversion after 30 minutes. The reaction was quenched with citric acid and water. The mixture was extracted with 3×EtOAc. Combined organic layers were dried over Na₂SO₄, and dry packed over silica gel. Purification from 0 to 10% MeOH in DCM over 12 CVs afforded 320 mg of the desired product 2′ in good purity (99.9%)

LCMS method 3: Retention time: 1.339 min, 99.9% purity at 215 nm, [M+H]⁺=499.3

Step 2′. Preparation of 3-[4-[4-[2-(4-Aminocyclohexyl)ethyl]piperazin-1-yl]phenyl]piperidine-2,6-dione;hydrochloride (3′): HCl (6.15 mL, 24.615 mmol, 42.0 eq.) was added to a suspension of tert-butyl N-[4-[2-[4-[4-(2,6-dioxo-3-piperidyl)phenyl]piperazin-1-yl]ethyl]cyclohexyl]carbamate 2 (320.0 mg, 0.586 mmol, 1.0 eq.) in DCM (4 mL, 0.15 M) at room temperature for 16 hours. The solvent was evaporated under vacuum, and the residual HCl was co-evaporated with MeCN (2×). The residue was dried under high vacuum to give 3-[4-[4-[2-(4-aminocyclohexyl)ethyl]piperazin-1-yl]phenyl]piperidine-2,6-dione hydrochloride 3′ (333 mg, quantitative yield) as a white solid.

LCMS method 1: retention time: 0.40 min, 99.9% purity at 215 nm, [M−2HCl+2H]²⁺=200.4.

¹H NMR (400 MHz, DMSO-d6) δ ppm 0.93-1.08 (m, 2H), 1.19-1.39 (m, 3H), 1.59-1.68 (m, 2H), 1.77 (br d, J=12.5 Hz, 2H), 1.89-2.04 (m, 3H), 2.07-2.20 (m, 1H), 2.42-2.48 (m, 1H), 2.58-2.69 (m, 1H), 2.85-2.98 (m, 1H), 3.04-3.21 (m, 6H), 3.54-3.57 (m, 2H), 3.73-3.82 (m, 3H), 6.96 (br d, J=8.8 Hz, 2H), 7.11 (d, J=8.6 Hz, 2H), 8.00 (br s, 3H), 10.78 (s, 1H).

Step 3′. Preparation of 3-[4-[4-[2-(4-Azidocyclohexyl)ethyl]piperazin-1-yl] phenyl] piperidine-2,6-dione (4′): The triflyl azide was prepared as follows: after NaN₃ (164.37 mg, 2.528 mmol, 11.0 eq.) was dissolved in water (1 mL), toluene (1 mL) was added. The mixture was cooled to 0° C. under vigorous stirring. After the dropwise addition of Tf₂O (0.21 mL, 1.264 mmol, 5.5 eq.) and further vigorous stirring for 30 minutes at 0° C., the temperature was raised to 10° C. and the biphasic mixture was stirred for 2 hours. A saturated aqueous solution of NaHCO₃ was added dropwise until gas evolution had ceased. The two phases were separated and the aqueous layer was extracted with toluene (2×1.6 mL). The combined organic layers were used as fallows. To a solution of 3-[4-[4-[2-(4-aminocyclohexyl)ethyl]piperazin-1-yl]phenyl]piperidine-2,6-dione hydrochloride 4′ (100.0 mg, mmol, 1.0 eq.) in water (1 mL), CuSO₄·5H₂O (8.61 mg, 0.034 mmol, 0.15 eq.), NaHCO₃ (154.5 mg, 1.839 mmol, 8.0 eq.) were added while stirring. The mixture was cooled in an ice bath and the above prepared solution of triflyl azide then added dropwise. The reaction mixture was allowed to warm to room temperature for 16 hours. The reaction was quenched using saturated aqueous solution of NH₄C₁. The product was extracted with DCM (3×). The combined organic phases were washed once with brine, dried over magnesium sulfate, filtered and concentrated to give 3-[4-[4-[2-(4-azidocyclohexyl)ethyl]piperazin-1-yl]phenyl]piperidine-2,6-dione 4 (86 mg, 80% yield) as a white solid.

LCMS method 1: retention time: 1.308 min, 90.2% purity at 215 nm, [M+H]⁺=425.4.

¹H NMR (400 MHz, DMSO-d6) δ ppm 0.80-0.89 (m, 1H), 0.96-1.08 (m, 2H), 1.19-1.30 (m, 5H), 1.33-1.41 (m, 2H), 1.74-1.81 (m, 2H), 1.88-1.95 (m, 2H), 1.96-2.02 (m, 1H), 2.07-2.16 (m, 2H), 2.31-2.38 (m, 2H), 2.57-2.68 (m, 2H), 3.03-3.14 (m, 5H), 3.72 (dd, J=10.9, 4.8 Hz, 1H), 6.88 (d, J=8.6 Hz, 2H), 7.04 (d, J=8.6 Hz, 2H), 10.76 (s, 1H).

Step 4. Preparation of 1-(4-(Cyclopentylamino)-5-(1-((1r,4r)-4-(2-(4-(4-(2,6-dioxopiperidin-3-yl)phenyl)piperazin-1-yl)ethyl)cyclohexyl)-1H-1,2,3-triazol-4-yl)pyridin-2-yl)-1H-pyrazolo[3,4-b]pyridine-5-carbonitrile (P-54): 1-[4-(cyclopentylamino)-5-ethynyl-2-pyridyl]pyrazolo[3,4-b]pyridine-5-carbonitrile A-9 (85.0 mg, 0.258 mmol, 1.0 eq.), 3-[4-[4-[2-(4-azidocyclohexyl)ethyl]piperazin-1-yl]phenyl]piperidine-2,6-dione 4′ (120.9 mg, 0.285 mmol, 1.1 eq.), CuSO₄ (16.53 mg, 0.1000 mmol) and sodium ascorbate (51.2 mg, 0.258 mmol, 1.0 eq.) were solubilized in water (0.9 mL), methanol (0.9 mL) and THF (0.9 mL, 0.1 M). The reaction was stirred at room temperature for 24 h. The reaction was stopped at 40% conversion into expected compound. Volatiles were evaporated and to the crude solution was added DMSO and the mixture was directly loaded on a 50 g C18 reverse-phase chromatography column using a dry pad (elution: 5% MeCN/0.1% HCOOH over 4 CV, then 5% to 55% MeCN/0.1% HCOOH over 15 CV, product exited at 35% MeCN). The pure fractions were combined, concentrated under reduced pressure and lyophilised overnight to afford 1-(4-(cyclopentylamino)-5-(1-((1r,4r)-4-(2-(4-(4-(2,6-dioxopiperidin-3-yl)phenyl)piperazin-1-yl)ethyl)cyclohexyl)-1H-1,2,3-triazol-4-yl)pyridin-2-yl)-1H-pyrazolo[3,4-b]pyridine-5-carbonitrile P-54 (20.7 mg, 10% yield) as a yellow solid.

LCMS method 2: retention time: 2.449 min, 97.9% purity at 215 nm, [M+H]⁺=753.4.

¹H NMR (400 MHz, DMSO-d6) δ ppm 0.77-0.91 (m, 1H), 1.08-1.32 (m, 4H), 1.36-1.75 (m, 9H), 1.77-1.95 (m, 4H), 1.96-2.24 (m, 6H), 2.38-2.46 (m, 1H), 2.56-2.70 (m, 3H), 2.97-3.22 (m, 4H), 3.74 (dd, J=10.6, 4.5 Hz, 1H), 4.41-4.65 (m, 2H), 6.92 (d, J=6.8 Hz, 2H), 7.07 (d, J=7.6 Hz, 2H), 7.55-7.84 (m, 1H), 8.39-9.90 (m, 6H), 10.77 (s, 1H).

Table 38 summarizes the compounds prepared using General Procedure X-9.

TABLE 38
Final Compounds Prepared via General Procedure X-9

| Com- | | | | |
|---|---|---|---|---|
| pound | TMB | CBM | | |
| No. | Portion | Portion | Structure | Characterization |
| P-54 | A9 | C12 | 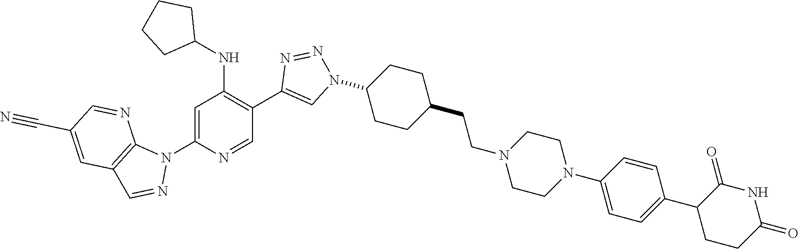 | 10% yield as a yellow solid. LCMS method 2: Retention time: 2.449 min, 97.9% purity at 215 nm, [M + H]⁺ = 753.4, [M + 2H]⁺ = 377.3 ¹H NMR (400 MHz, DMSO-d6) δ ppm 0.77-0.91 (m, 1 H), 1.08-1.32 (m, 4 H), 1.36-1.75 (m, 9 H), 1.77-1.95 (m, 4 H), 1.96-2.24 (m, 6 H), 2.38-2.46 (m, 1 H), 2.56- 2.70 (m, 3 H), 2.97-3.22 (m, 4 H), 3.74 (dd, J = 10.6, 4.5 Hz, 1 H), 4.41-4.65 (m, 2 H), 6.92 (d, J = 6.8 Hz, 2 H), 7.07 (d, J = 7.6 Hz, 2 H), 7.55-7.84 (m, 1 H), 8.39-9.90 (m, 6 H), 10.77 (s, 1 H). One signal overlaps with the DMSO signal in ¹H NMR. One proton was not apparent in ¹H NMR. |
| P-195 | A77 | C12 | 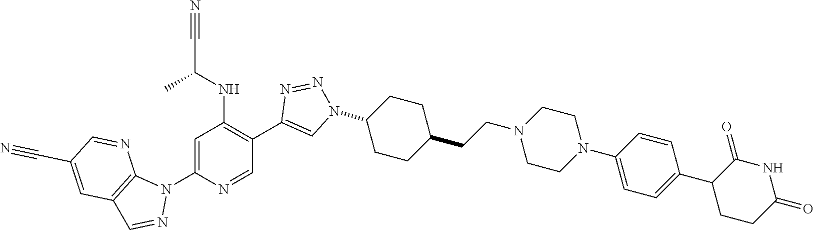 Use of A-77 in Step 4 | 38% yield as an off-white solid. LCMS method 8: Retention time: 1.889 min, 96.5% purity at 215 nm, [M + H]⁺ = 738.2 ¹H NMR (400 MHz, DMSO-d6): δ ppm 1.27-1.31 (m, 2H), 1.43-1.48 (m, 1H), 1.67-1.69 (m, 2H), 1.75 (d, J = 7.2 Hz, 3H), 1.88-2.04 (m, 6H), 2.14-2.28 (m, 3H), 2.61- 2.66 (m, 1H), 2.95-3.01 (m, 2H), 3.16-3.26 (m, 4H), 3.75- 3.79 (m, 2H), 3.83-3.87 (m, 2H), 4.60-4.64 (m, 1H), 5.04- 5.08 (m, 1 H), 6.99 (d, J = 8.8 Hz, 2H), 7.13 (d, J = 8.8 Hz, 2H), 7.54 (s, 1H), 8.63 (d, J = 7.2 Hz, 1H), 8.69 (s, 1 H), 8.76 (s, 1H), 8.94 (s, 1H), 9.05 (s, 2H), 10.79 (s, 1H). One proton was not apparent by ¹H NMR. |

General Procedure X-10. The scheme shown below for the synthesis of P-81 is provided as a representative synthesis for General Procedure X-10.

### Example S49. 6-(5-cyano-1H-pyrazolo[3,4-b]pyridin-1-yl)-4-(cyclopentylamino)-N-((1r,4r)-4-(2-(4-(4-(2,6-dioxopiperidin-3-yl)phenyl)piperazin-1-yl)-2-methylpropyl)cyclohexyl)nicotinamide (P-81)

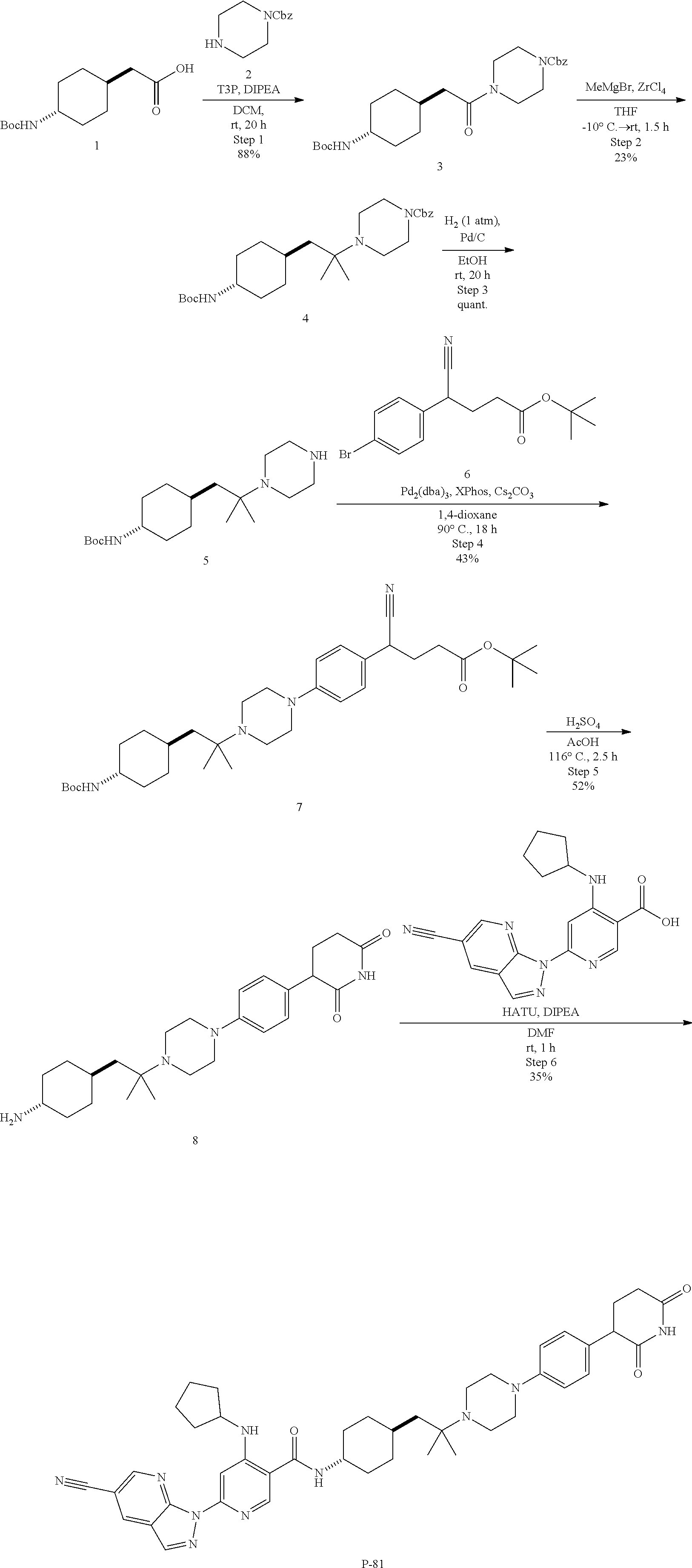

Step 1. Preparation of benzyl 4-[2-[4-(tert-butoxycarbonylamino)cyclohexyl]acetyl]piperazine-1-carboxylate (3): 2-[4-(tert-Butoxycarbonylamino)cyclohexyl]acetic acid (1) (1.0 g, 3.89 mmol, 1.0 equiv) was suspended in DCM (20 mL), then DIPEA (2.03 mL, 11.7 mmol, 3.0 equiv) and T3P (3.01 mL, 5.05 mmol, 1.3 equiv) were added sequentially. The reaction was stirred for 5 minutes at room temperature, then benzyl piperazine-1-carboxylate (2) (942 mg, 4.27 mmol, 1.1 equiv) was added and the resulting solution was stirred for 20 hours at room temperature. The reaction was quenched with the addition of saturated NaHCO_{3(aq)} (20 mL), then the aqueous phase was extracted with DCM (3×15 mL). The organic layers were combined, dried over MgSO₄, filtered and evaporated under reduced pressure. The crude residue was purified by normal phase flash chromatography (DCM/MeOH, 100:0 to 95:5, 80 g RediSep Rf Gold® Normal-Phase Silica, 20 CV, λ=254-280 nm) to afford 3 (1.575 g, 3.42 mmol, 88% yield) as a white solid.

LCMS method 1: 99.9% purity at 215 nm, [M+H]⁺=404.2.

¹H NMR (400 MHz, CDCl₃) δ ppm 0.99-1.20 (m, 4H), 1.44 (s, 9H), 1.72-1.87 (m, 3H), 1.95-2.06 (m, 2H), 2.16-2.26 (m, 2H), 3.30-3.42 (m, 1H), 3.43-3.57 (m, 6H), 3.62 (br s, 2H), 4.28-4.46 (m, 1H), 5.16 (s, 2H), 7.30-7.45 (m, 5H).

Step 2. Preparation of benzyl 4-[2-[4-(tert-butoxycarbonylamino)cyclohexyl]-1,1-dimethyl-ethyl]piperazine-1-carboxylate (4): A solution of benzyl 4-[2-[4-(tert-butoxycarbonylamino)cyclohexyl]acetyl]piperazine-1-carboxylate (3) (1.675 g, 3.64 mmol, 1.0 equiv) in THF (85 mL) was cooled to −10° C. (internal temperature as monitored with a thermocouple) then zirconium(IV) chloride (1.03 g, 4.41 mmol, 1.2 equiv) was added in one portion. The reaction was stirred for 30 minutes at −10° C., then 3.0 M methylmagnesium bromide in diethyl ether (7.9 mL, 23.7 mmol, 6.5 equiv) was added dropwise over 5 minutes (internal temperature kept below −6° C. throughout the addition). The reaction was allowed to stir for 10 minutes at −10° C., then the ice-bath was removed and the reaction was stirred for 1.5 hours at room temperature. The reaction was quenched with the addition of saturated NH₄Cl_{(aq)} (40 mL), then diluted with DCM (150 mL). The mixture was filtered over celite, then the celite was washed with DCM (100 mL). The phases were separated and the aqueous layer was extracted with DCM (2×50 mL). The organic layers were combined, dried over MgSO₄, filtered and evaporated under reduced pressure. The crude residue was purified by reversed-phase flash chromatography (MeOH in 0.1% HCOOH(aq), 5% (3 CV)→80%, 150 g RediSep Rf Gold® C18Aq, 18 CV, λ=214-254 nm). The fractions containing the product were combined and concentrated under reduced pressure. The aqueous solution was treated with 1.0 M NaOH(aq) and extracted with DCM (3×25 mL), then the combined organic layers were dried over MgSO₄ and evaporated under reduced pressure to yield 4 (396 mg, 0.835 mmol, 23% yield) as a white foam.

LCMS method 1: 99.9% purity at 215 nm, [M+H]⁺=474.3.

¹H NMR (400 MHz, CDCl₃) δ ppm 0.99 (s, 6H), 1.02-1.16 (m, 4H), 1.29 (br d, J=5.0 Hz, 2H), 1.41 (br s, 1H), 1.45 (s, 9H), 1.83 (br d, J=9.3 Hz, 2H), 1.95 (br d, J=6.7 Hz, 2H), 2.49 (br s, 4H), 3.34 (br s, 1H), 3.40-3.53 (m, 4H), 4.34 (br s, 1H), 5.13 (s, 2H), 7.29-7.41 (m, 5H).

Step 3. Preparation of tert-butyl N-[4-(2-methyl-2-piperazin-1-yl-propyl)cyclohexyl]carbamate (5): Benzyl 4-[2-[4-(tert-butoxycarbonylamino)cyclohexyl]-1,1-dimethyl-ethyl]piperazine-1-carboxylate (4) (395 mg, 0.834 mmol, 1.0 equiv) was dissolved in ethanol (20 mL), then the solution was degassed by bubbling nitrogen under sonication for 20 minutes. Pd/C 5% w/w (177 mg, 0.0834 mmol, 0.1 equiv) was added, then the mixture was further degassed by bubbling nitrogen under sonication for 20 minutes. The nitrogen balloon was replaced with one filled with hydrogen, which was bubbled through the reaction mixture for minutes, then the reaction mixture was stirred under static hydrogen atmosphere overnight. The reaction mixture was filtered through celite to remove the catalyst, then the celite was washed thoroughly with DCM. The solvent was evaporated under reduced pressure to yield 5 (283 mg, 0.834 mmol, quantitative yield) as a grey oil.

LCMS method 1: 99.9% purity at 215 nm, [M+H]⁺=340.3.

¹H NMR (400 MHz, CDCl₃) δ ppm 1.00 (s, 6H), 1.02-1.16 (m, 4H), 1.29 (d, J=Hz, 2H), 1.35-1.42 (m, 1H), 1.45 (s, 9H), 1.78-1.80 (m, 1H), 1.83 (br d, J=9.9 Hz, 2H), 1.95 (br d, J=9.5 Hz, 2H), 2.55 (br s, 4H), 2.89 (br t, J=4.2 Hz, 4H), 3.34 (br s, 1H), 4.35 (br s, 1H).

Step 4. Preparation of tert-butyl 4-[4-[4-[2-[4-(tert-butoxycarbonylamino)cyclohexyl]-1,1-dimethyl-ethyl]piperazin-1-yl]phenyl]-4-cyano-butanoate (7): In a sealed tube, tert-butyl 4-(4-bromophenyl)-4-cyano-butanoate (6) (179 mg, mmol, 1.1 equiv), tert-butyl N-[4-(2-methyl-2-piperazin-1-yl-propyl)cyclohexyl]carbamate (5) (170 mg, 0.501 mmol, 1.0 equiv), cesium carbonate (270 mg, mmol, 1.65 equiv) and XPhos (41.6 mg, 0.0873 mmol, 0.17 equiv) were sparged with nitrogen, then anhydrous 1,4-dioxane (5 mL) was added and nitrogen was bubbled trough the mixture for 15 minutes under sonication. Pd₂(dba)₃CHCl₃ (39.8 mg, 0.0385 mmol, 0.077 equiv) was then quickly added and nitrogen bubbling was continued for additional 5 minutes. The tube was sealed and the reaction mixture was stirred at 90° C. overnight. The reaction mixture was cooled to room temperature, diluted with EtOAc (20 mL), and filtered through celite, then the celite was washed exhaustively with EtOAc. The volatiles were evaporated under reduced pressure and the residue was purified by reversed-phase flash chromatography (MeOH in 0.1% HCOOH_{(aq)}, 5% (3 CV)→60%, 50 g RediSep Rf Gold® C18Aq, 18 CV, λ=214-254 nm, product with 60% MeOH). The fractions containing the product were combined and concentrated under reduced pressure. The aqueous solution was treated with saturated NaHCO_{3(aq)} and extracted with DCM (3×20 mL), then the combined organic layers were dried over MgSO₄ and evaporated under reduced pressure to yield 7 (125 mg, 0.214 mmol, 43% yield) as a yellow solid.

LCMS method 1: 99.9% purity at 215 nm, [M+H]⁺=583.4.

¹H NMR (400 MHz, CDCl₃) δ ppm 1.04 (s, 6H), 1.06-1.16 (m, 4H), 1.34 (br d, J=4.9 Hz, 2H), 1.37-1.42 (m, 1H), 1.42-1.50 (m, 18H), 1.79-1.90 (m, 2H), 1.92-2.03 (m, 2H), 2.07-2.22 (m, 2H), 2.26-2.49 (m, 2H), 2.62-2.77 (m, 4H), 3.08-3.23 (m, 4H), 3.34 (br s, 1H), 3.87 (t, J=7.3 Hz, 1H), 4.34 (br s, 1H), 6.90 (d, J=8.7 Hz, 2H), 7.20 (d, J=8.7 Hz, 2H).

Step 5. Preparation of 3-[4-[4-[2-(4-aminocyclohexyl)-1,1-dimethyl-ethyl]piperazin-1-yl]phenyl]piperidine-2,6-dione hydrochloride (8HCl): tert-butyl 4-[4-[4-[2-[4-(tert-butoxycarbonylamino)cyclohexyl]-1,1-dimethyl-ethyl]piperazin-1-yl]phenyl]-4-cyano-butanoate (7) (125 mg, 0.214 mmol, 1.0 equiv) was dissolved in acetic acid (1 mL), then sulfuric acid (46.2 μL, 0.867 mmol, 4.0 equiv) was added dropwise and the mixture was stirred at 118° C. for 2.5 hours. Acetic acid was co-evaporated with toluene (3×5 mL) and the residue was dried under high vacuum. The residue was dissolved in the minimum amount of water and purified by reversed-phase flash chromatography (MeCN in 0.02 M HCl(aq), 0% (5 CV)→20%, 30 g Claricep™ Spherical AQ C18, 20 CV, λ=214-254 nm) to afford 8·HCl (52.0 mg, 0.112 mmol, 52% yield) as a white solid.

LCMS method 1: 99.9% purity at 215 nm, [M+H]⁺=427.3.

¹H NMR (400 MHz, DMSO-d6) δ ppm 1.01-1.17 (m, 2H), 1.27-1.39 (m, 3H), 1.40 (s, 6H), 1.62 (br s, 2H), 1.81 (br d, J=11.2 Hz, 2H), 1.91 (br d, J=10.8 Hz, 2H), 1.96-2.06 (m, 1H), 2.06-2.24 (m, 1H), 2.45 (br d, J=4.6 Hz, 1H), 2.58-2.75 (m, 1H), 2.85-2.99 (m, 1H), 3.09-3.18 (m, 2H), 3.19-3.36 (m, 2H), 3.56 (br d, J=11.2 Hz, 2H), 3.70-3.91 (m, 3H), 6.96 (d, J=8.6 Hz, 2H), 7.12 (d, J=8.4 Hz, 2H), 7.71-8.24 (m, 3H), 10.79 (s, 1H).

Step 6. Preparation of 6-(5-cyano-1H-pyrazolo[3,4-b]pyridin-1-yl)-4-(cyclopentylamino)-N-((1r,4r)-4-(2-(4-(4-(2,6-dioxopiperidin-3-yl)phenyl)piperazin-1-yl)-2-methylpropyl)cyclohexyl)nicotinamide (P-81): 6-(5-Cyanopyrazolo[3,4-b]pyridin-1-yl)-4-(cyclopentylamino)pyridine-3-carboxylic acid (9) (34.0 mg, 0.0976 mmol, 1.0 equiv) and 3-[4-[4-[2-(4-aminocyclohexyl)-1,1-dimethyl-ethyl]piperazin-1-yl]phenyl]piperidine-2,6-dione hydrochloride (8·HCl) (52.0 mg, 0.112 mmol, 1.15 equiv) were dissolved in DMF (1.0 mL), then DIPEA (255 μL, 1.46 mmol, 15.0 equiv) and HATU (55.7 mg, 0.146 mmol, 1.5 equiv) were added in sequence. The reaction mixture was stirred for one hour at room temperature. The reaction mixture was injected directly in a column for reversed-phase flash chromatography purification (MeCN in 0.1% HCOOH_{(aq)}, 5% (3 CV)→80%, 50 g RediSep Rf Gold® C18Aq, 15 CV, λ=214-254 nm). The fractions containing the product were evaporated to dryness, then co-evaporated with water (3×10 mL) to completely remove any trace of residual HCOOH, and the residue was freeze-dried overnight to yield P-81 (26.53 mg, 0.0340 mmol, 35% yield) as a white solid.

LCMS method 2: 97.1% purity at 215 nm, [M+H]⁺=757.4, [M+2H]²⁺=379.3.

¹H NMR (400 MHz, DMSO-d6) δ ppm 1.02 (s, 6H), 1.05-1.16 (m, 2H), 1.23-1.40 (m, 4H), 1.44-1.55 (m, 3H), 1.58-1.76 (m, 4H), 1.76-1.94 (m, 4H), 1.95-2.21 (m, 4H), 2.39-2.48 (m, 1H), 2.57-2.71 (m, 5H), 2.99-3.18 (m, 4H), 3.61-3.79 (m, 2H), 3.83-3.99 (m, 1H), 6.88 (d, J=8.6 Hz, 2H), 7.04 (d, J=8.6 Hz, 2H), 7.32 (s, 1H), 8.31 (d, J=7.8 Hz, 1H), 8.52-8.67 (m, 3H), 9.01 (d, J=2.0 Hz, 1H), 9.04 (d, J=2.0 Hz, 1H), 10.76 (s, 1H).

Table 39 summarizes the compounds prepared using General Procedure X-10.

TABLE 39
Final Compounds Prepared via General Procedure X-10

| Com- | | | | |
|---|---|---|---|---|
| pound | TMB | CBM | | |
| No. | Portion | Portion | Structure | Characterization |
| P-81 | A7 | | 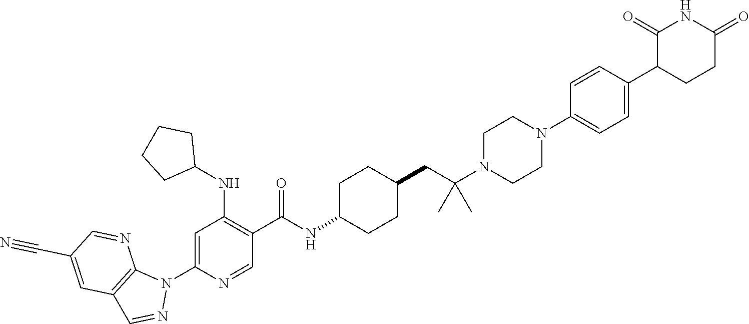 | 34% yield as an white solid. LCMS method 5: Retention time: 2.473 min, 97.1% purity at 215 nm, [M + H]⁺ = 757.4, [M + 2H]²⁺ = 379.3 ¹H NMR (400 MHz, DMSO-d6) δ ppm 1.02 (s, 6 H), 1.05-1.16 (m, 2 H), 1.23-1.40 (m, 4 H), 1.44-1.55 (m, 3 H), 1.58-1.76 (m, 4 H), 1.76-1.94 (m, 4 H), 1.95-2.21 (m, 4 H), 2.39-2.48 (m, 1 H), 2.57-2.71 (m, 5 H), 2.99-3.18 (m, 4 H), 3.61-3.79 (m, 2 H), 3.83-3.99 (m, 1 H), 6.88 (d, J = 8.6 Hz, 2 H), 7.04 (d, J = 8.6 Hz, 2 H), 7.32 (s, 1 H), 8.31 (d, J = 7.8 Hz, 1 H), 8.52-8.67 (m, 3 H), 9.01 (d, J = 2.0 Hz, 1 H), 9.04 (d, J = 2.0 Hz, 1 H), 10.76 (s, 1 H). |

### General Procedure X-11

### Route to Intermediate alkyne L-2

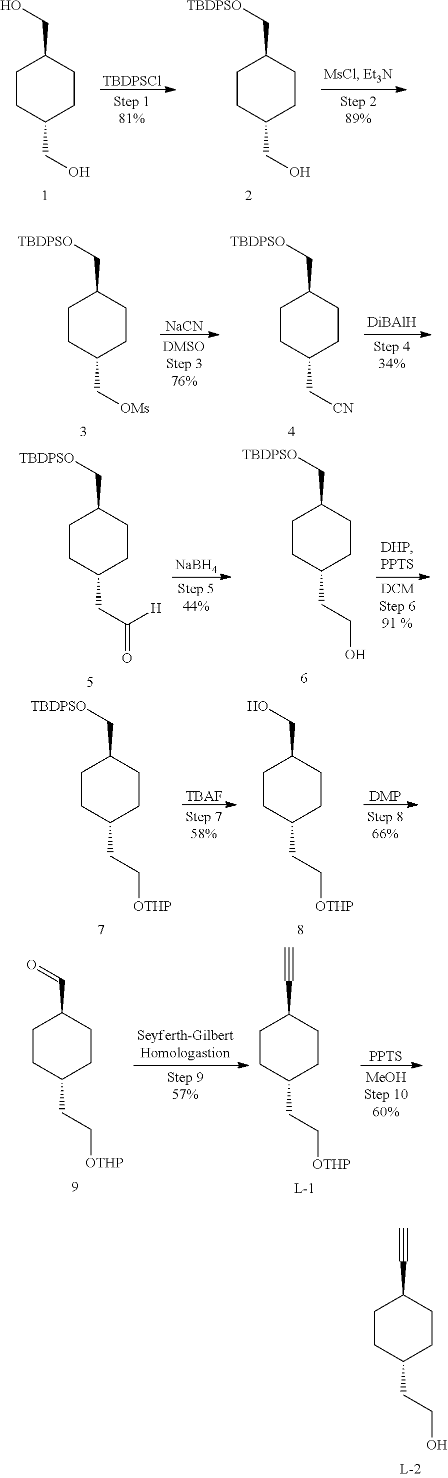

Step 1. Preparation of [4-[[tert-butyl)diphenyl)silyl]oxymethyl] cyclohexyl] methanol (2): To a solution of [4-(hydroxymethyl)cyclohexyl]methanol 1 (3 g, 20.8 mmol, 1 eq.) in dry DMF (83.2 mL, 0.25 M) was added imidazole (0.85 g, 12.48 mmol, 0.6 eq.), followed by tert-butylchlorodiphenylsilane (2.7 mL, 10.4 mmol, 0.5 eq.). After stirring for 18 h at room temperature, TLC (25% EtOAc in Heptanes, revealing UV+KMnO₄) showed full conversion. To the reaction mixture was added half-brine solution (40 mL). The aqueous phase was extracted with EtOAc (2×50 mL). The organics were washed with half-brine (4×25 mL), dried over Na₂SO₄ and concentrated to dryness. The residue was dry-packed and purified by normal phase flash chromatography (80 g silica column, elution: 0 to 30% EtOAc/Heptane over 10 CV, product came out around 20% EtOAc). Fractions were combined and concentrated to give 2 (6.48 g, 43% yield) as a colorless oil.

¹H NMR (400 MHz, chloroform-d) δ ppm 0.82-1.02 (m, 4H), 1.03-1.07 (m, 9H), 1.38-1.55 (m, 2H), 1.66-1.88 (m, 4H), 2.04 (br d, J=3.2 Hz, 2H), 3.42-3.49 (m, 3H), 7.32-7.44 (m, 6H), 7.65 (d, J=6.6 Hz, 3H), 7.70-7.75 (m, 1H).

Step 2. Preparation of [4-[[tert-butyl)diphenyl)silyl]oxymethyl]cyclohexyl]methyl methanesulfonate (3): To a round bottom flask was added [4-[[tert-butyl(diphenyl)silyl]oxymethyl]cyclohexyl]methanol 2 (6.48 g, 8.93 mmol, 1 eq.) in DCM (44.65 mL, 0.2 M). The flask was then cooled to 0° C., then methanesulfonyl chloride (0.76 mL, 9.82 mmol, 1.1 eq.) and triethylamine (1.62 mL, 11.61 mmol, 1.3 eq.) were added. After 2 h, TLC (Heptanes/EtOAc; 10/90) showed complete conversion of the starting material into the desired product (KMnO₄ revelator). The mixture was partitioned between EtOAc and water. The organic phase was washed once with water and once with 1 N HCl, dried over magnesium sulfate, filtered and evaporated under reduced pressure, affording 3 (3.65 g, 89% yield) as a colorless solid. The crude was used directly as is for the next reaction.

¹H NMR (400 MHz, chloroform-d) δ ppm 1.03 (br s, 3H), 1.07 (br d, J=12.7 Hz, 9H), 1.45-1.56 (m, 1H), 1.64-1.77 (m, 1H), 1.83-1.90 (m, 3H), 3.01 (s, 3H), 3.48 (d, J=6.1 Hz, 2H), 4.05 (d, J=6.6 Hz, 2H), 7.35-7.47 (m, 6H), 7.64-7.69 (m, 3H), 7.71-7.79 (m, 1H). Two protons were missing by ¹H NMR.

Step 3. Preparation of 2-[4-[[tert-butyl)diphenyl)silyl]oxymethyl]cyclohexyl]acetonitrile (4): To a round bottom flask were added [4-[[tert-butyl(diphenyl)silyl]oxymethyl]cyclohexyl]methyl methanesulfonate 3 (3.65 g, 7.92 mmol, 1 eq.) and NaCN (1.19 g, 18.21 mmol, 2.3 eq.) in DMSO (12.2 mL, 0.65 M). The reaction mixture was then stirred at 50° C. After an overnight period, TLC (Heptanes/EtOAc; showed complete conversion of the starting material into compound 4 (KMnO₄ revelator). The reacting mixture was poured into an erlenmeyer of crushed ice and stirred until all ice was melted, the resulting solid was isolated by filtration and dissolved in a minimum of Ethyl acetate, washed with NaHCO₃ and brine and evaporated to dryness. The residue was dry-packed and purified by normal phase flash chromatography (80 g silica column, elution: 5 to 100% EtOAc/Heptane over 15 CV). Fractions were combined and concentrated to give 4 (2.35 g, 76% yield) as a colorless oil.

¹H NMR (400 MHz, chloroform-d) δ ppm 0.83-0.91 (m, 1H), 0.98-1.05 (m, 2H), 1.05-1.09 (m, 9H), 1.09-1.18 (m, 2H), 1.46-1.56 (m, 1H), 1.58-1.65 (m, 1H), 1.89 (br t, J=11.9 Hz, 3H), 2.27 (d, J=6.6 Hz, 2H), 3.48 (d, J=5.9 Hz, 2H), 7.37-7.46 (m, 6H), 7.64-7.69 (m, 3H), 7.71-7.75 (m, 1H).

Step 4. Preparation of 2-[4-[[tert-butyl)diphenyl)silyl]oxymethyl]cyclohexyl]acetaldehyde (5): To a round bottom flask was added 2-[4-[[tert-butyl(diphenyl)silyl]oxymethyl] cyclohexyl] acetonitrile 4 (2.35 g, 6 mmol, 1 eq.) in DCM (10.7 mL, 0.56 M), then the reaction mixture was cooled at −78° C. To the cooled reaction mixture was then added DIBAL-H (1 M solution in DCM) (17.99 mL, 17.99 mmol, 3 eq.) and the reaction stirred at −78° C. After 4 h, 40 mL of 4M HCl were added carefully and the solution was slowly warmed to RT. Stirring was continued at RT for 10 min, and the substance was partitioned between ethyl acetate and 1 M HCl. The organic phases were washed with water, dried over magnesium sulfate and concentrated under reduced pressure. The residue was dry-packed and purified by normal phase flash chromatography (40 g silica column, elution: 1:3 Hept.:EtOAc). Fractions were combined and concentrated to give 5 (803 mg, 34% yield) as a colorless oil that was used as is for the next reaction.

Step 5. Preparation of 2-[4-[[tert-butyl(diphenyl)silyl]oxymethyl]cyclohexyl]ethanol (6): To a round bottom flask was added 2-[4-[[tert-butyl(diphenyl)silyl]oxymethyl]cyclohexyl]acetaldehyde 5 (800 mg, 2.03 mmol, 1 eq.) in EtOH (10.2 mL, 0.2 M) at 0° C. To the reaction mixture was added NaBH₄ (192.26 mg, mmol, 2.5 eq.) and the reaction stirred at 0° C. After 4 h, rochelle salt solution was added at then the solution was heated up to room temperature. Organic layers were extracted twice with EtOAc and the latter was washed with Brine twice and dried over MgSO₄. The residue was dry-packed and purified by normal phase flash chromatography (40 g silica column, elution: 0 to % EtOAc/Heptane over 15 CV, product came out around 20% EtOAc). Fractions were combined and concentrated to give 6 (353.7 mg, 44% yield) as a colorless oil.

¹H NMR (400 MHz, chloroform-d) δ ppm 0.93-1.03 (m, 4H), 1.06 (s, 9H), 1.13-1.20 (m, 1H), 1.31-1.41 (m, 1H), 1.50 (q, J=6.8 Hz, 3H), 1.74-1.86 (m, 4H), 3.47 (d, J=6.1 Hz, 2H), 3.67-3.73 (m, 2H), 7.36-7.45 (m, 6H), 7.67 (dd, J=7.8, 1.5 Hz, 4H).

Step 6. Preparation of tert-butyl-diphenyl-[[4-(2-tetrahydropyran-2-yloxyethyl)cyclohexyl]methoxy]silane (7): To a solution of 2-[4-[[tert-butyl(diphenyl)silyl]oxymethyl]cyclohexyl]ethanol 6 (1.62 g, 4.08 mmol, 1 eq.) in DCM (25.06 mL, 0.16 M) was added PPTS (205.28 mg, 0.82 mmol, 0.2 eq.) and DHP (0.93 mL, 10.21 mmol, 2.5 eq.). The reaction was stirred at room temperature. After 24 h, TLC showed complete conversion of the starting material 6′. The reaction mixture was concentrated to dryness, and the residue was dry-packed and purified by normal phase flash chromatography (80 g silica column, elution: 0 to 30% EtOAc/Heptane over 15 CV, product came out around 20% EtOAc). Fractions were combined and concentrated to give 7 (1.79 g, 91% yield) as a colorless oil.

¹H NMR (400 MHz, chloroform-d) δ ppm 0.84-0.91 (m, 3H), 0.93-1.00 (m, 3H), 1.03-1.11 (m, 9H), 1.29-1.39 (m, 2H), 1.50-1.55 (m, 3H), 1.56-1.63 (m, 2H), 1.70 (br s, 5H), 3.40-3.45 (m, 1H), 3.47 (d, J=6.1 Hz, 2H), 3.48-3.55 (m, 1H), 3.76-3.84 (m, J=9.7, 7.2, 7.2 Hz, 1H), 3.84-3.92 (m, 1H), 4.55-4.61 (m, 1H), 7.35-7.45 (m, 6H), 7.64-7.76 (m, 4H).

Step 7. Preparation of [4-(2-tetrahydropyran-2-yloxyethyl)cyclohexyl]methanol (8): To a stirred solution of tert-butyl-diphenyl-[[4-(2-tetrahydropyran-2-yloxyethyl)cyclohexyl]methoxy]silane 7 (1.79 g, 3.72 mmol, 1 eq.) in THF (4.65 mL, 0.8 M) was added 1 M TBAF solution in THF (14.89 mL, 14.89 mmol, 4 eq.) at room temperature. After 5 h, TLC showed complete conversion of the starting material 7′. Solvents were removed under reduced pressure and the residue was purified by normal phase flash chromatography (80 g gold column, solid deposit, elution 0 to 40% EtOAc/Heptane over 10 CV)(CAM was used as the TLC stain). Fractions were combined and concentrated to give 8 (577.5 mg, 58% yield) as a colorless oil.

¹H NMR (400 MHz, chloroform-d) δ ppm 0.90-1.01 (m, 4H), 1.33-1.46 (m, 2H), 1.48-1.63 (m, 7H), 1.68-1.76 (m, 1H), 1.77-1.89 (m, 5H), 3.39-3.54 (m, 4H), 3.77-3.91 (m, 2H), 4.56-4.60 (m, 1H).

Step 8. Preparation of 4-(2-tetrahydropyran-2-yloxyethyl)cyclohexanecarbaldehyde (9)

To a stirred solution of [4-(2-tetrahydropyran-2-yloxyethyl)cyclohexyl]methanol 8 (704.5 mg, 2.91 mmol, 1 eq.) in DCM (36.34 mL, 0.04 M) and THF (36.34 mL, 0.04 M) was added DMP (2.47 g, 5.81 mmol, 2 eq.) and Water (3 drops) at 0° C. After 2.5 h, TLC showed complete conversion of the starting material 8′. Solvents were removed under reduced pressure without a heating bath, taken up with EtOAc, washed with sat. NaHCO₃, extracted with EtOAc (3×), and then washed with brine. The combined organic layers were dried over Na₂SO₄, filtered and concentrated under reduced pressure. The residue was then purified by normal phase flash chromatography (24 g column, solid deposit, elution 0 to 30% EtOAc/Heptane over 10 CV). Fractions were combined and concentrated to give 9 (459.5 mg, 66% yield) as a colorless solid.

¹H NMR (400 MHz, DMSO-d6) δ ppm 0.92-1.01 (m, 2H), 1.08-1.22 (m, 2H), 1.26-1.36 (m, 1H), 1.38-1.53 (m, 6H), 1.55-1.64 (m, 1H), 1.65-1.83 (m, 3H), 1.84-1.94 (m, 2H), 2.15-2.25 (m, 1H), 3.32-3.45 (m, 2H), 3.62-3.77 (m, 2H), 4.49-4.56 (m, 1H), 9.55 (d, J=1.0 Hz, 1H).

Step 9. Preparation of 2-[2-(4-ethynylcyclohexyl)ethoxy]tetrahydropyran (L-1)

To a flame-dried round bottom flask were added 4-(2-tetrahydropyran-2-yloxyethyl)cyclohexanecarbaldehyde 9 (459.5 mg, 1.91 mmol, 1 eq.) and K₂CO₃ (494.18 mg, 3.82 mmol, 2 eq.). Vacuum was applied and the flask was then filled with nitrogen (repeated three times). Then, MeOH (9.56 mL, 0.2 M) was added and the reaction mixture stirred at room temperature under nitrogen for 20 minutes. Then, Bestmann-Ohira (4.4 mL, 2.29 mmol, 1.2 eq.) (10% in acetonitrile) was added to the reaction mixture. The resulting mixture was stirred at room temperature under a nitrogen atmosphere. After 4 h, TLC (eluting 10% EtOAc in Heptanes) showed complete conversion of starting material 9. The residue was dry-packed and then purified by normal phase flash chromatography (24 g column, solid deposit, elution 0 to 10% EtOAc/Heptane over 10 CV. Fractions were combined and concentrated to give L-1 (275.7 mg, 57% yield) as a colorless oil.

¹H NMR (400 MHz, chloroform-d) δ ppm 0.88-0.98 (m, 2H), 1.33-1.45 (m, 3H), 1.48-1.62 (m, 7H), 1.68-1.76 (m, 1H), 1.77-1.88 (m, 3H), 1.95-2.03 (m, 2H), 2.14-2.24 (m, 1H), 3.36-3.56 (m, 2H), 3.75-3.90 (m, 2H), 4.55-4.59 (m, 1H).

Step 10. Preparation of 4-(2-tetrahydropyran-2-yloxyethyl)cyclohexanecarbaldehyde (L-2): To a stirred solution of 2-[2-(4-ethynylcyclohexyl)ethoxy]tetrahydropyran L-1 (50 mg, 0.2 mmol, 1 eq.) in MeOH (1.97 mL, 0.1 M) was added PTSA (3.39 mg, 0.02 mmol, 0.1 eq.) at room temperature. After 1 h, TLC showed complete conversion of the starting material 10′. The reaction mixture was quenched with triethylamine and the mixture was concentrated under reduced pressure. The residue was then purified by normal phase flash chromatography (24 g gold column, solid deposit, elution 0 to 30% EtOAc/Heptane over 10 CV)(CAM was used as a TLC revelator). Fractions were combined and concentrated to give L-2 (18 mg, 60% yield) as a colorless oil.

¹H NMR (400 MHz, chloroform-d) δ ppm 0.88-1.01 (m, 2H), 1.30-1.50 (m, 6H), 1.73-1.82 (m, 2H), 1.95-2.03 (m, 2H), 2.03-2.07 (m, 1H), 2.13-2.24 (m, 1H), 3.68 (t, J=6.6 Hz, 2H).

### Route to Final Product X-11

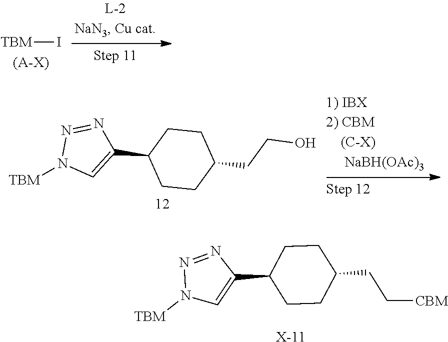

Step 11. Preparation of 1-[4-(cyclopentylamino)-5-[4-[4-(2-hydroxyethyl)cyclohexyl]triazol-1-yl]-2-pyridyl]pyrazolo[3,4-b]pyridine-5-carbonitrile (12): To a round bottom flask were added 2-(4-ethynylcyclohexyl)ethanol L-1 (1 eq.), TBM A-X (1 eq.), sodium ascorbate (0.4 eq.), CuI (0.35 eq.), trans-N,N′-Dimethylcyclohexane-1,2-diamine (0.5 eq.) and NaN₃ (2 eq.) in DMSO (0.15 M). The reaction mixture was then stirred at room temperature. After an overnight period, LCMS showed complete conversion and the crude mixture was purified by reverse phase purification. Fractions were combined and concentrated, affording 12 that was used as is for the next step.

Step 12. Preparation of 1-[4-(cyclopentylamino)-5-[4-[4-[2-[4-[4-(2,6-dioxo-3-piperidyl)phenyl]piperazin-1-yl]ethyl]cyclohexyl]triazol-1-yl]-2-pyridyl]pyrazolo[3,4-b]pyridine-5-carbonitrile (X-11): To a solution of 1-[4-(cyclopentylamino)-5-[4-[4-(2-oxoethyl)cyclohexyl]triazol-1-yl]-2-pyridyl]pyrazolo[3,4-b]pyridine-5-carbonitrile 12 (1 eq.) in dry DMSO (0.3 M) was added IBX (1.3 eq.). The resulting mixture was stirred at room temperature overnight. The solution of aldehyde was added dropwise over 3 minutes to a solution of CBM C-X (1 eq.), DCM (0.3 M) and DIPEA (5 eq.). The mixture was stirred at RT for 10 minutes, then NaBH(OAc)₃ (1.3 eq.) was added. After LCMS showed full conversion, the DCM was evaporated and the reaction mixture was directly loaded for reverse phase purification. Pure fractions were combined and concentrated to give X-11.

### Example S50. 1-[4-(cyclopentylamino)-5-[4-[4-[2-[4-[4-(2,6-dioxo-3-piperidyl)phenyl]piperazin-1-yl]ethyl]cyclohexyl]triazol-1-yl]-2-pyridyl]pyrazolo[3,4-b]pyridine-5-carbonitrile (P-87)

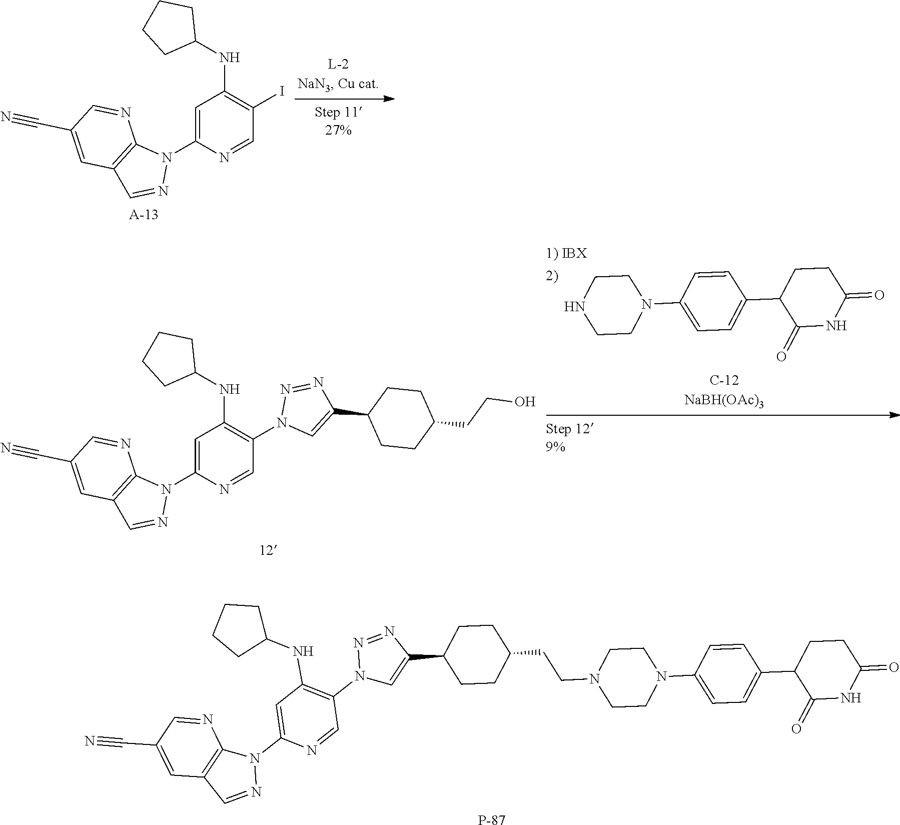

Step 11. Preparation of 1-[4-(cyclopentylamino)-5-[4-[4-(2-hydroxyethyl)cyclohexyl]triazol-1-yl]-2-pyridyl]pyrazolo[3,4-b]pyridine-5-carbonitrile (12′): To a round bottom flask were added 2-(4-ethynylcyclohexyl)ethanol L-2 (97.5 mg, 0.64 mmol, 1 eq.), 1-[4-(cyclopentylamino)-5-iodo-2-pyridyl]pyrazolo[3,4-b]pyridine-5-carbonitrile A-13 (275.57 mg, 0.64 mmol, 1 eq.), sodium ascorbate (50.73 mg, 0.26 mmol, 0.4 eq.), CuI (42.69 mg, 0.22 mmol, 0.35 eq.), trans-N,N′-Dimethylcyclohexane-1,2-diamine (50.5 uL, 0.32 mmol, 0.5 eq.) and NaN₃ (83.27 mg, 1.28 mmol, 2 eq.) in DMSO (4.27 mL, 0.15 M). The reaction mixture was then stirred at room temperature. After an overnight period, LCMS showed complete conversion into compound 13. The crude mixture was purified by reverse phase FC purification (50 g C18 column, liquid deposit (DMSO), 5% MeCN/0.1% HCOOH over 5 CV, then 5 to 100% MeCN/0.1% HCOOH over 15 CV, product came out around 50% MeCN). Fractions were combined and concentrated, affording 12′ (87.45 mg, 27% yield) as a yellow solid that was used as is for the next step. LCMS showed good amount of the desired product.

LCMS method 2: 90.9% purity at 254 nm, [M+H]⁺=498.3.

Step 12. Preparation of 1-[4-(cyclopentylamino)-5-[4-[4-[2-[4-[4-(2,6-dioxo-3-piperidyl)phenyl]piperazin-1-yl]ethyl]cyclohexyl]triazol-1-yl]-2-pyridyl]pyrazolo[3,4-b]pyridine-5-carbonitrile (P-87): To a solution of 1-[4-(cyclopentylamino)-5-[4-[4-(2-oxoethyl)cyclohexyl]triazol-1-yl]-2-pyridyl]pyrazolo[3,4-b]pyridine-5-carbonitrile 12′ (81.47 mg, 0.16 mmol, 1 eq.) in dry DMSO (0.26 mL, 0.3 M) was added IBX (59.84 mg, 0.21 mmol, 1.3 eq.). The resulting mixture was stirred at room temperature overnight. The solution of aldehyde was added dropwise over 3 minutes to a solution of 3-(4-piperazin-1-ylphenyl)piperidine-2,6-dione;hydrochloride C-12 (50.93 mg, 0.16 mmol, 1 eq.), DCM (0.26 mL, 0.3 M) and DIPEA (0.14 mL, 0.82 mmol, 5 eq.). The mixture was stirred at RT for 10 minutes, then NaBH(OAc)₃ (45.29 mg, 0.21 mmol, 1.3 eq.) was added. After 16 h, LCMS showed full conversion into compound P-87. The DCM was evaporated and the reaction mixture was directly loaded for reverse phase FC purification (50 g C18 column, liquid deposit (DMSO), 5% MeCN/0.1% HCOOH over 4 CV, then 5 to 100% MeCN/0.1% HCOOH over 15 CV, product came out at 35% MeCN). All tubes were impure, so they were combined, affording the desired product with 50% purity (IBX residue). Another purification by reverse phase FC purification was made (100 g C18 column, liquid deposit (DMSO), 5% MeCN/0.1% HCOOH over 4 CV, then 5 to 100% MeCN/0.1% HCOOH over 15 CV, product came out at 35% MeCN). Pure fractions were combined and concentrated to give P-87 (10.93 mg, 9% yield) as a yellow solid.

LCMS method 2: 98.3% purity at 215 nm, [M+H]⁺=753.5.

¹H NMR (400 MHz, DMSO-d6) δ ppm 0.98-1.28 (m, 3H), 1.29-1.75 (m, 11H), 1.78-1.95 (m, 2H), 1.96-2.07 (m, 3H), 2.09-2.22 (m, 3H), 2.25-2.48 (m, 4H), 2.55-2.79 (m, 3H), 3.01-3.22 (m, 4H), 3.43-3.60 (m, 1H), 3.72 (dd, J=10.5, 4.6 Hz, 1H), 3.89-4.00 (m, 1H), 6.76-6.85 (m, 1H), 6.89 (d, J=8.3 Hz, 2H), 7.05 (d, J=8.3 Hz, 2H), 7.53 (s, 1H), 8.35-8.41 (m, 1H), 8.41-8.46 (m, 1H), 8.67 (s, 1H), 9.01-9.05 (m, 1H), 9.05-9.10 (m, 1H), 10.77 (s, 1H).

### Example S51. 1-(4-(((R)-1-cyanoethyl)amino)-5-(4-((1r,4R)-4-(2-(4-(4-(2,6-dioxopiperidin-3-yl)phenyl) piperazin-1-yl)ethyl)cyclohexyl)-1H-1,2,3-triazol-1-yl)pyridin-2-yl)-1H-pyrazolo[3,4-b]pyridine-5-carbonitrile, formic acid salt (P-139)

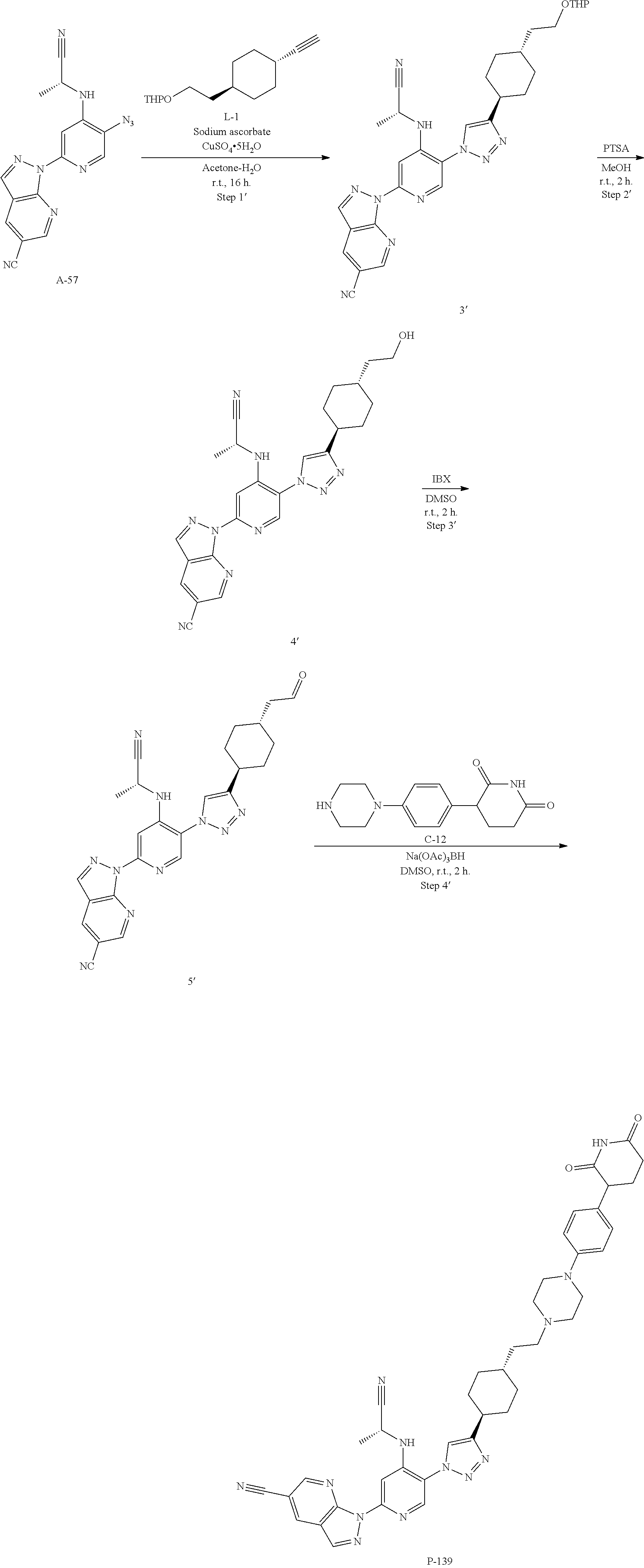

LCMS Method 1. Kinetex XB-C18, 75×3.0 mm, 2.6 Temperature: RT, Flow: 1.0 mL/min, Run Time: 5 min, Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 100% Mobile Phase B for 4.0 min. MSD positive.

Step 1′. Preparation of 1-(4-(((R)-1-cyanoethyl)amino)-5-(4-((1r,4R)-4-(2-((tetrahydro-2H-pyran-2-yl)oxy)ethyl)cyclohexyl)-1H-1,2,3-triazol-1-yl)423yridine-2-yl)-1H-pyrazolo[3,4-b]pyridine-5-carbonitrile 3′: To a stirred solution of (R)-1-(5-azido-4-((1-cyanoethyl)amino)423yridine-2-yl)-1H-pyrazolo[3,4-b]pyridine-5-carbonitrile A-57 (430 mg, 1.302 mmol) in acetone (6.0 mL) was added 2-(2-((1r,4r)-4-ethynylcyclohexyl)ethoxy)tetrahydro-2H-pyran L-1 (338 mg, 1.432 mmol) followed by sodium ascorbate (129 mg, 0.651 mmol) and then a solution of copper(II) sulphate pentahydrate (65 mg, mmol) in H₂O (1.0 mL) was added at room temperature. The reaction mixture was stirred at room temperature for 16 h. The reaction mixture was treated with water (100 mL) and extracted with ethyl acetate (3×75 mL). The combined organic layer was washed with brine (10 mL), dried over sodium sulphate and concentrated under reduced pressure to give the crude product. The crude compound was purified by column chromatography using silica gel (230-400 mesh) with 80-90% ethyl acetate/pet ether to obtain 1-(4-(((R)-1-cyanoethyl)amino)-5-(4-((1r,4R)-4-(2-((tetrahydro-2H-pyran-2-yl)oxy)ethyl)cyclohexyl)-1H-1,2,3-triazol-1-yl)424yridine-2-yl)-1H-pyrazolo[3,4-b]pyridine-5-carbonitrile 3′ (220 mg, 20.2% yield).

LCMS method 1: retention time 3.078 min, 67.93% purity at 220 nm, [M+H]⁺=567.0.

Step 2′. Preparation of 1-(4-(((R)-1-cyanoethyl)amino)-5-(4-((1r,4R)-4-(2-hydroxyethyl)cyclohexyl)-1H-1,2,3-triazol-1-yl)pyridin-2-yl)-1H-pyrazolo[3,4-b]pyridine-5-carbonitrile 4′: To a stirred solution of 1-(4-(((R)-1-cyanoethyl)amino)-5-(4-((1r,4R)-4-(2-((tetrahydro-2H-pyran-2-yl)oxy)ethyl)cyclohexyl)-1H-1,2,3-triazol-1-yl)pyridin-2-yl)-1H-pyrazolo[3,4-b]pyridine-5-carbonitrile 3′ (220 mg, 0.388 mmol) in MeOH (4.0 mL), pTSA (73.8 mg, 0.388 mmol) was added at RT and stirred for 2 h. The reaction mixture was treated saturated sodium bicarbonate solution (30 mL) and extracted with DCM (2×40 mL). The organic layer was washed with brine (25.0 mL), dried over sodium sulphate, and concentrated under reduced pressure to give crude product 1-(4-(((R)-1-cyanoethyl)amino)-5-(4-((1r,4R)-4-(2-hydroxyethyl)cyclohexyl)-1H-1,2,3-triazol-1-yl)pyridin-2-yl)-1H-pyrazolo[3,4-b]pyridine-5-carbonitrile 4′ (130 mg) as a brown solid, which was used in the next step without further pufication.

LCMS method 1: retention time 1.994 min, [M+H]⁺=483.2.

Step 3′. Preparation of 1-(4-(((R)-1-cyanoethyl) amino)-5-(4-((1r,4R)-4-(2-oxoethyl) cyclohexyl)-1H-1,2,3-triazol-1-yl) pyridin-2-yl)-1H-pyrazolo[3,4-b]pyridine-5-carbonitrile 5′: IBX (151 mg, 0.539 mmol, 2.0 eq.) was added to a solution of 1-(4-(((R)-1-cyanoethyl) amino)-5-(4-((1r,4R)-4-(2-hydroxyethyl) cyclohexyl)-1H-1,2,3-triazol-1-yl) pyridin-2-yl)-1H-pyrazolo[3,4-b]pyridine-5-carbonitrile 4′ (130 mg, 0.269 mmol, 1.0 eq.) in DMSO (3 mL) at RT. The resulting solution was stirred for 2 h at RT. The reaction mixture was diluted ethyl acetate (15 mL) and washed with ice-cold water, aqueous sodium bicarbonate solution followed by brine solution, dried over sodium sulphate, and concentrated under reduced pressure to give the crude product 1-(4-(((R)-1-cyanoethyl) amino)-5-(4-((1r,4R)-4-(2-oxoethyl) cyclohexyl)-1H-1,2,3-triazol-1-yl) pyridin-2-yl)-1H-pyrazolo[3,4-b]pyridine-5-carbonitrile 5′ (90 mg) as a brown solid, which was used in the next step without further purification.

LCMS method 1: retention time: 2.297 min, [M+H]⁺=481.0.

Step 4′. Preparation of 1-(4-(((R)-1-cyanoethyl)amino)-5-(4-((1r,4R)-4-(2-(4-(4-(2,6-dioxopiperidin-3-yl)phenyl) piperazin-1-yl)ethyl)cyclohexyl)-1H-1,2,3-triazol-1-yl)pyridin-2-yl)-1H-pyrazolo[3,4-b]pyridine-5-carbonitrile, formic acid salt P-139: To a solution of 1-(4-(((R)-1-cyanoethyl)amino)-5-(4-((1r,4R)-4-(2-oxoethyl)cyclohexyl)-1H-1,2,3-triazol-1-yl)pyridin-2-yl)-1Hpyrazolo[3,4-b]pyridine-5-carbonitrile 5′ (90 mg, 0.187 mmol, 1.0 eq) and 3-(4-(piperazin-1-yl)phenyl)piperidine-2,6-dione 2 HCl C-12 (78 mg, 0.225 mmol, 1.2 eq) in anhydrous DMSO (4 mL) was added sodium triacetoxyborohydride (119 mg, 0.562 mmol, 2.0 eq) under nitrogen atmosphere at room temperature. The resulting mixture was stirred at room temperature for 2 h. The reaction mixture was treated with water (25 mL) and extracted with DCM (2×25 mL). Organic phases were combined and washed with brine (25 mL). Combined organic phases were dried over anhydrous Na₂SO₄, filtered and concentrated under reduced pressure to give the crude product. The crude was purified by Preparative HPLC to afford 1-(4-(((R)-1-cyanoethyl)amino)-5-(4-((1r,4R)-4-(2-(4-(4-(2,6-dioxopiperidin-3-yl)phenyl)piperazin-1-yl)ethyl)cyclohexyl)-1H-1,2,3-triazol-1-yl)pyridin-2-yl)-1H-pyrazolo[3,4-b]pyridine-5-carbonitrile, formic acid salt P-139 (19 mg, 0.023 mmol, 12.21% yield) as an off-white solid. Prep-HPLC method: X-Select C18, 150×19 mm, 5 Mobile phase: A: 0.1% HCOOH in MQ-water; B: Acetonitrile; Flow rate: 15 mL/min

LCMS method 1: retention time: 1.717 min, 98.5% purity at 220 nm, [M+H]⁺=738.0.

¹H NMR (400 MHz, DMSO-d6): δ ppm 1.12-1.15 (m, 2H), 1.38-1.53 (m, 5H), 1.61 (d, J=7.2 Hz, 3H), 1.87-1.90 (m, 2H), 1.98-2.03 (m, 1H), 2.07-2.15 (m, 3H), 2.37-2.44 (m, 2H), 2.59-2.64 (m, 1H), 2.71-2.74 (m, 1H), 3.12-3.17 (m, 4H), 3.71-3.75 (m, 1H), 4.99-5.03 (m, 1H), 6.89 (d, J=8.8 Hz, 2H), 7.05 (d, J=8.8 Hz, 2H), 7.20 (d, J=7.6 Hz, 1H), 7.67 (s, 1H), 8.35 (s, 1H), 8.35 (s, 1H), 8.41 (s, 1H), 9.05-9.07 (m, 2H), 10.77 (s, 1H).

Table 40 summarizes the compounds prepared using General Procedure X-11.

TABLE 40
Final Compounds Prepared via General Procedure X-11

| Compound | TMB | CBM | | |
|---|---|---|---|---|
| No. | Portion | Portion | Structure | Characterization |
| P-87 | A13 | C12 | 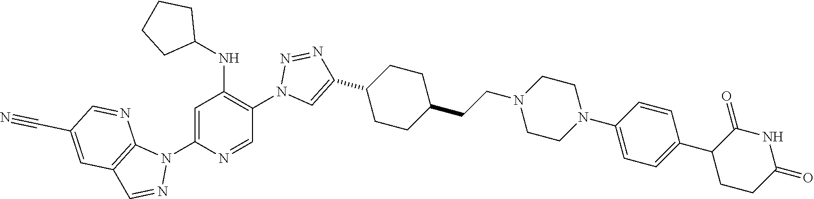 | 8.7% yield as a yellow solid. LCMS method 2: Retention time: 2.662 min, 98.3% purity at 215 nm, [M + H]⁺ = 753.5, [M + 2H]⁺ = 377.3 ¹H NMR (400 MHz, DMSO-d6) δ ppm 0.98-1.28 (m, 3 H), 1.29- 1.75 (m, 11 H), 1.78 0.195 (m, 2 H), 1.96-2.07 (m, 3 H), 2.09- 2.22 (m, 3 H), 2.25-2.46 (m, 4 H), 2.55-2.79 (m, 3 H), 3.01- 3.22 (m, 4 H), 3.43-3.60 (m, 1 H), 3.72 (dd, J = 10.5, 4.6 Hz, 1 H), 3.89-4.00 (m, 1 H), 6.76-6.85 (m, 1 H), 6.89 (d, J = 8.3 Hz, 2 H), 7.05 (d, J = 8.3 Hz, 2 H), 7.53 (s, 1 H), 8.34-8. 41 (m, 1 H), 8.41- 8.46 (m, 1 H) 8.67 (s, 1 H), 9.01- 9.05 (m, 1 H), 9.05-9.10 (m, 1 H), 10.77 (s, 1 H). |
| P-139 | A57 | C12 | 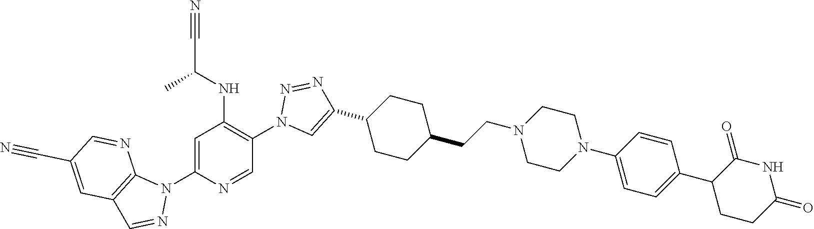 | 12% yield as an off-white solid. LCMS method 7: Retention time: 1.717 min, 94.3% purity at 215 nm, [M + H]⁺ = 738.0 ¹H NMR (400 MHz, DMSO-d6): δ ppm 1.12-1.15 (m, 2H), 1.38- 1.53 (m, 5H), 1.61 (d, J = 7.2 Hz, 3H), 1.87-1.90 (m, 2H), 1.98- 2.03 (m, 1H), 2.07-2.15 (m, 3H), 2.37-2.44 (m, 2H), 2.59-2.64 (m, 1H), 2.71-2.74 (m, 1H), 3.12- 3.17 (m, 4H), 3.71-3.75 (m, 1H), 4.99-5.03 (m, 1H), 6.89 (d, J = 8.8 Hz, 2H), 7.05 (d, J = 8.8 Hz, 2H), 7.20 (d, J = 7.6 Hz, 1H), 7.67 (s, 1H), 8.35 (s, 1H), 8.35 (s, 1H), 8.41 (s, 1H), 9.05-9.07 (m, 2H), 10.77 (s, 1H). |
| P-140 | A58 | C12 | 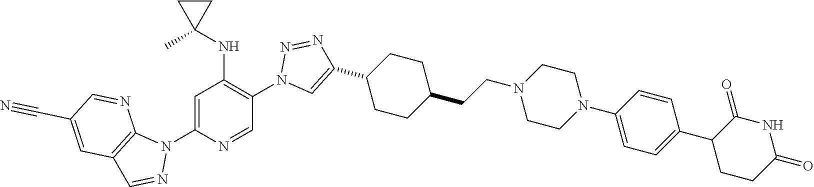 | 31% yield as an off-white solid. LCMS method 7: Retention time: 1.980 min, 92.8% purity at 215 nm, [M + H]⁺ = 739.0 ¹H-NMR (400 MHz, DMSO-d6, 80° C.): δ ppm 0.82-0.88 (m, 4H), 1.12-1.21 (m, 2H), 1.45 (s, 3H), 1.45-1.57 (m, 5H), 1.89- 1.92 (m, 2H), 2.07-2.17 (m, 4H), 2.56-2.79 (m, 5H), 3.12-3.17 (m, 4H), 3.72-3.76 (m, 1H), 6.90 (d, J = 8.4 Hz, 2H), 7.03 (s, 1H), 7.08 (d, J = 8.4 Hz, 2H), 7.88 (s, 1H), 8.24 (s, 1H), 8.33 (s, 1H), 8.64 (s, 1H), 8.98 (d, J = 1.2 Hz, 1H), 9.03 (d, J = 1.2 Hz, 1H), 10.47 (s, 1H). Six protons were not apparent by ¹H NMR. |
| P-154 | A63 | C12 | 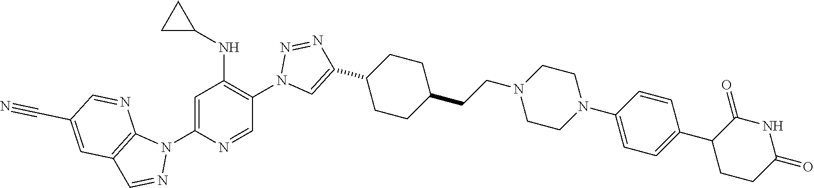 | 25% yield as an off-white solid. LCMS method 7: Retention time: 1.860 min, 95.6% purity at 215 nm, [M + H]⁺ = 725.2 ¹H NMR (400 MHz, DMSO-d6, 80 C): δ ppm 0.57-0.59 (m, 2H), 0.83-0.85 (m, 2H), 1.11-1.15 (m, 2H), 1.39-1.51 (m, 6H), 1.85- 1.88 (m, 2H), 2.06-2.13 (m, 5H), 2.41-2.48 (m, 2H), 2.51-2.55 (m, 4H), 2.57-2.75 (m, 2H), 3.12- 3.14 (m, 4H), 3.70-3.71 (m, 1H), 6.87-6.89 (m, 3H), 7.06 (d, J = 8.4 Hz, 2H), 7.87 (s, 1H), 8.20 (s, 1H), 8.30 (s, 1H), 8.61 (s, 1H), 8.92 (d, J = 1.6 Hz, 1H), 8.98 (d, J = 2.0 Hz, 1H). |
| P-155 | A57 | C43 | 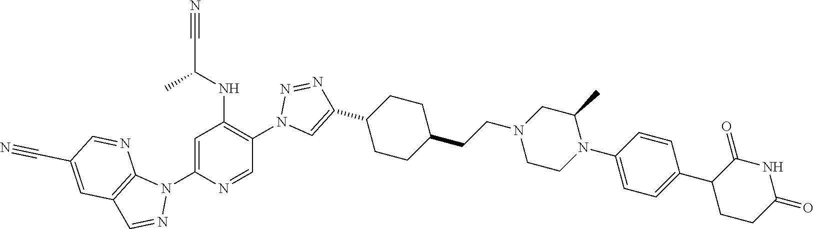 | 31% yield as an off-white solid. LCMS method 7: Retention time: 1.462 min, 99.4% purity at 215 nm, [M + H]⁺ = 725.2 ¹H NMR (400 MHz, DMSO-d6, 80° C.): δ ppm 1.01-1.28 (m, 6H), 1.45-1.58 (m, 4H), 1.66 (d, J = 6.8 Hz, 3H), 1.70-1.73 (m, 2H), 1.91-1.94 (m, 2H), 2.10-2.21 (m, 4H), 2.63-2.68 (m, 1H), 2.78- 2.82 (m, 1H), 3.10-3.63 (m, 8H), 3.78-3.83 (m, 1H), 4.95-4.99 (m, 1H), 6.96-7.17 (m, 5H), 7.73 (s, 1H), 8.33 (s, 1H), 8.45 (s, 1H), 8.67 (s, 1H), 8.99-9.03 (m, 2H), 10.52 (s, 1H). |
| P-156 | A57 | C44 | 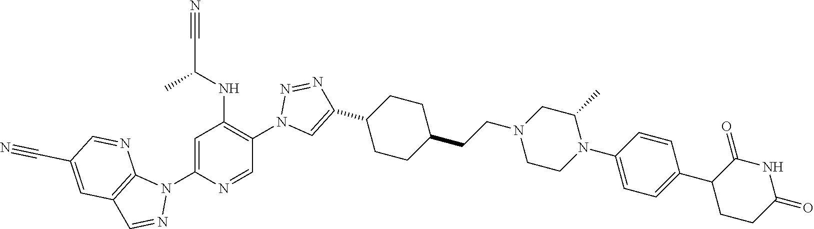 | 29% yield as an off-white solid. LCMS method 7: Retention time: 1.455 min, 96.4% purity at 215 nm, [M + H]⁺ = 725.2 ¹H NMR (400 MHz, DMSO-d6): δ ppm 1.02 (d, J = 6.4 Hz, 3H), 1.13-1.16 (m, 2H), 1.44-1.50 (m, 5H), 1.62 (d, J = 6.8 Hz, 3H), 1.88-1.91 (m, 2H), 2.03-2.16 (m, 5H), 2.26-2.44 (m, 3H), 2.59- 2.75 (m, 4H), 2.86-2.95 (m, 2H), 3.24-3.27 (m, 1H), 3.70-3.73 (m, 1H), 3.95-3.99 (m, 1H), 4.98- 5.04 (m, 1H), 6.84 (d, J = 8.8 Hz, 2H), 7.05 (d, J = 8.8 Hz, 2H), 7.20 (d, J = 8.4 Hz, 1H), 7.67 (s, 1H), 8.36 (s, 1H), 8.41 (s, 1H), 8.72 (s, 1H), 8.99-9.08 (m, 2H), 10.77 (s, 1H) |
| P-169 | A69 | C12 | 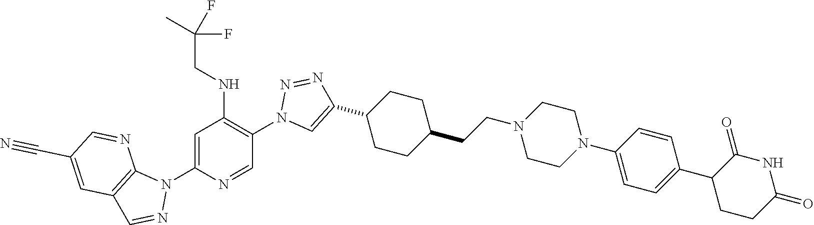 | 10% yield as an off-white solid. LCMS method 7: Retention time: 1.516 min, 95.9% purity at 215 nm, [M + H]⁺ = 763.2 ¹H NMR (400 MHz, DMSO-d6): δ 1.18-1.24 (m, 2H), 1.38-1.53 (m, 3H), 1.63-1.73 (m, 5H), 1.88- 1.91 (m, 2H), 2.01-2.03 (m, 1H), 2.15-2.18 (m, 3H), 2.71-2.78 (m, 1H), 2.96-2.99 (m, 2H), 3.14-3.43 (m, 6H), 3.61-3.64 (m, 2H), 3.75- 3.86 (m, 5H), 6.99 (d, J = 8.8 Hz, 2H), 7.07-7.14 (m, 3H), 7.63 (s, 1H), 8.37 (s, 1H), 8.41 (s, 1H), 8.69 (s, 1H), 9.05-9.07 (m, 2H), 10.81 (s, 1H) |
| P-174 | A70 | C12 | 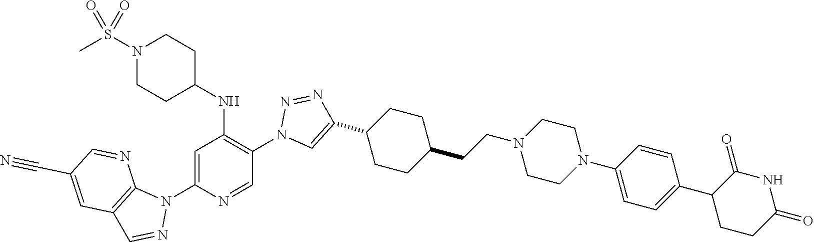 | 12% yield as an off-white solid. LCMS method 7: Retention time: 1.340 min, 95.1% purity at 215 nm, [M + H]⁺ = 846.2 ¹H NMR (400 MHz, DMSO-d6): δ ppm 1.11-1.25 (m, 2H), 1.38- 1.40 (m, 1H), 1.45-1.70 (m, 6H), 1.85-1.91 (m, 2H), 2.05-2.18 (m, 6H), 2.67-2.69 (m, 1H), 2.90 (s, 3H), 2.91-3.02 (m, 3H), 3.15-3.28 (m, 5H), 3.57-3.61 (m, 4H), 3.66- 3.69 (m, 2H), 6.73 (d, J = 7.2 Hz, 1H), 6.99 (d, J = 8.8 Hz, 2H), 7.13 (d, J = 8.8 Hz, 2H), 7.61 (s, 1H), 8.35 (s, 1H), 8.41 (s, 1H), 8.68 (s, 1H), 9.05 (d, J = 2.0 Hz, 1H), 9.09 (d, J = 2.0 Hz, 1H), 10.79 (s, 1H). Four protons were not apparent by ¹H NMR. |
| P-175 | A71 | C12 | 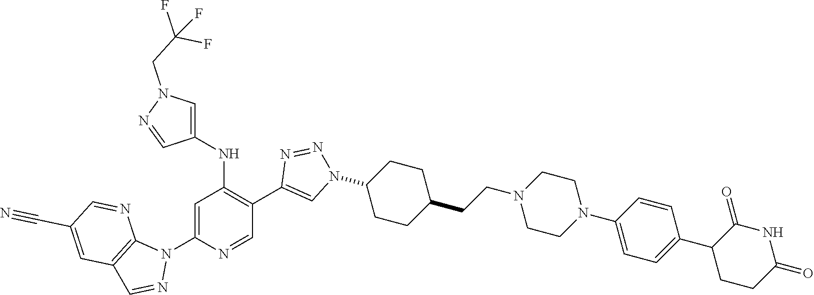 | 10% yield as an off-white solid. LCMS method 8: Retention time: 1.959 min, 95.6% purity at 215 nm, [M + H]⁺ = 833.1 ¹H NMR (400 MHz, DMSO-d6): δ ppm 1.16-1.21 (m, 2H), 1.38- 1.56 (m, 3H), 1.65-1.72 (m, 2H), 1.88-1.92 (m, 1H), 1.98-2.06 (m, 1H), 2.12-2.19 (m, 3H), 2.72-2.80 (m, 1H), 2.92-3.01 (m, 2H), 3.15-3.29 (m, 4H), 3.75-3.87 (m, 3H), 5.14-5.20 (m, 2H), 6.99 (d, J = 8.40 Hz, 2H), 7.13 (d, J = 8.40 Hz, 2H), 7.66 (s, 1H), 7.69 (s, 1H), 8.00 (s, 1H), 8.32-8.38 (m, 3H), 8.66 (s, 1H), 9.04 (s, 2H), 10.81 (s, 1H). Five protons were not apparent by ¹H NMR. |
| P-176 | C72 | C12 | 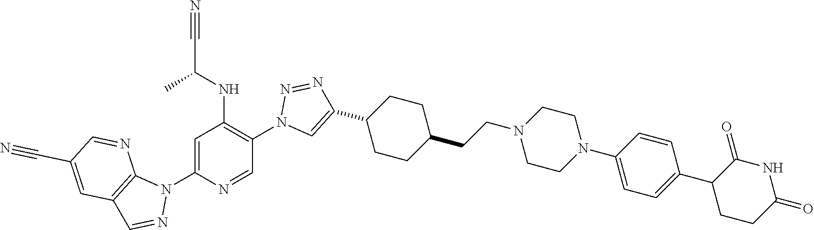 | 47% yield as an off-white solid. LCMS method 7: Retention time: 1.773 min, 98.7% purity at 215 nm, [M + H]⁺ = 737.2 ¹H NMR (400 MHz, DMSO-d6): δ ppm 1.12-1.21 (m, 2H), 1.35- 1.52 (m, 3H), 1.61-1.68 (m, 6H), 1.87-1.90 (m, 2H), 1.94-2.03 (m, 1H), 2.14-2.17 (m, 3H), 2.72-2.78 (m, 1H), 2.93-2.99 (m, 2H), 3.14-3.29 (m, 4H), 3.60-3.66 (m, 2H), 3.75-3.86 (m, 3H), 4.91-4.96 (m, 1H), 6.95-7.00 (m, 3H), 7.13 (d, J = 8.4 Hz, 2H), 7.18-7.20 (m, 1H), 7.34 (s, 1H), 7.35 (s, 1H), 7.47 (s, 1H), 8.54 (d, J = 4.0 Hz, 1H), 8.73 (d, J = 1.6 Hz, 1H), 8.82 (d, J = 2.0 Hz, 1H), 10.81 (s, 1H). One proton was not apparent by ¹H NMR. |
| P-201 | A57 | C31 | 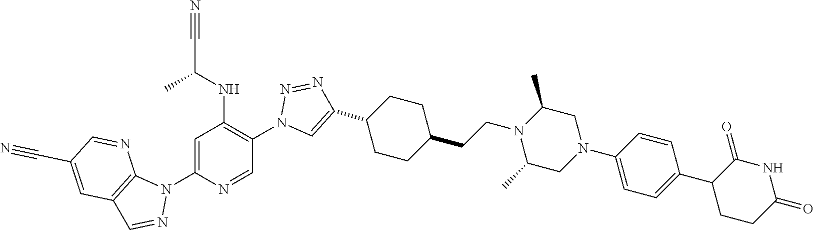 | 33% yield as a brown solid. LCMS method 7: Retention time: 1.537 min, 99.5% purity at 215 nm, [M + H]⁺ = 766.4 ¹H NMR (400 MHz, DMSO-d6): δ ppm 1.18-1.25 ( m, 2H), 1.37- 1.40 (m, 6H), 1.46-1.60 (m, 3H), 1.61 (d, J = 6.8 Hz, 3H), 1.62- 1.67 (m, 2H), 1.90-2.03 (m, 2H), 2.12-2.20 (m, 4H), 2.43- 2.48 (m, 1H), 2.63-2.70 (m, 1H), 2.75- 2.82 (m, 2H), 3.02-3.19 (m, 2H), 3.34-3.41 (m, 1H), 3.58-3.62 (m, 1H), 3.68-3.80 (m, 3H), 3.85-3.92 (m, 1 H), 5.01-5.05 (m, 1H), 6.98 (d, J = 8.8 Hz, 2H), 7.11 (d, J = 8.8 Hz, 2H), 7.20 (d, J = 8.4 Hz, 1H), 7.68 (s, 1H), 8.38 (s, 1H), 8.41 (s, 1H), 8.73 (s, 1H), 9.06-9.08 (m, 2H), 10.80 (s, 1H) |
| P-204 | A-57 | C-73 | 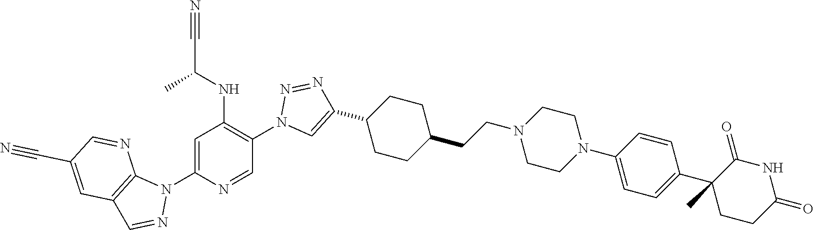 | 55% yield as an off-white solid. LCMS method 7: Retention time: 1.530 min, 99.1% purity at 215 nm, [M + H]⁺ = 752.3 ¹H NMR (400 MHz, DMSO-d6): δ ppm 1.17-1.23 (m, 2H), 1.40 (s, 3H), 1.45-1.55 (m, 2H), 1.60- 1.68 (m, 5H), 1.87-1.90 (m, 2H), 2.04-2.18 (m, 4H), 2.45-2.48 (m, 1H), 2.72-2.78 (m, 1H), 2.95-3.01 (m, 2H), 3.10-3.25 (m, 4H), 3.60-3.63 (m, 2H), 3.84- 3.87 (m, 2H), 5.01-5.07 (m, 1H), 7.02 (d, J = 9.20 Hz, 2H ), 7.17- 7.21 (m, 3H), 7.69 (s, 1H), 8.37 (s, 1H), 8.41 (s, 1H), 8.73 (s, 1H), 9.06-9.08 (m, 2H), 10.89 (s, 1H). Two protons were not apparent by ¹H NMR. |
| P-205 | A29 | C12 | 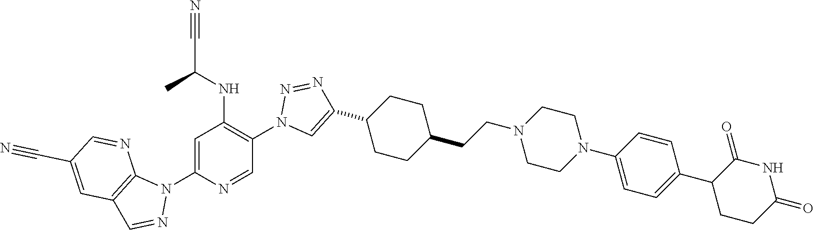 | 34% yield as a brown solid. LCMS method 7: Retention time: 1.689 min, 94.1% purity at 215 nm, [M + H]⁺ = 738.4 ¹H NMR (400 MHz, DMSO-d6): δ ppm 1.17-1.23 (m, 2H), 1.38- 1.53 (m, 3H), 1.61 (d, J = 7.20 Hz, 3H), 1.62-1.67 (m, 2H), 1.87- 1.91(m, 2H), 1.99-2.02 (m, 1H), 2.15-2.19 (m, 3H), 2.46-2.47 (m, 1H), 2.60-2.68 (m, 1H), 2.75- 2.79 (m, 1H), 2.94-3.00 (m, 2H), 3.15-3.25 (m, 4H), 3.61-3.64 (m, 2H), 3.75-3.87 (m, 3H), 5.01-5.05 (m, 1H), 6.99 (d, J = 8.80 Hz, 2H), 7.13 (d, J = 8.40 Hz, 2H), 7.21 (d, J = 8.0 Hz, 1H), 7.68 (s, 1H), 8.38 (s, 1H), 8.41 (s, 1H), 8.73 (s, 1H), 9.06-9.08 (m, 2H), 10.81 (s, 1H). |
| P-206 | A57 | C-74 | 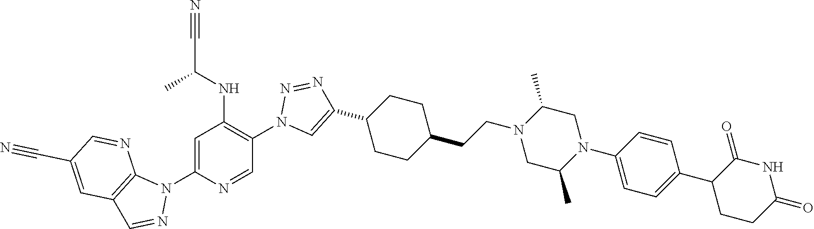 | 22% yield as an off-white solid. LCMS method 7: Retention time: 1.480 min, 95.0% purity at 215 nm, [M + H]⁺ = 766.4 ¹H NMR (400 MHz, DMSO-d6): δ ppm 0.96-0.99 (m, 6H), 1.12- 1.19 (m, 2H), 1.41-1.51 (m, 5H), 1.62 (d, J = 6.8 Hz, 3H), 1.88- 1.91 (m, 2H), 2.01-2.26 (m, 6H), 2.61-2.84 (m, 5H), 3.06-3.09 (m, 2H), 3.53-3.57 (m, 1H), 3.72- 3.77 (m, 1H), 4.97-5.01 (m, 1H), 6.93 (d, J = 8.40 Hz, 2H), 7.07 (d, J = 8.40 Hz, 2H), 7.20 (d, J = 8.40 Hz, 1H), 7.67 (s, 1H), 8.36 (s, 1H), 8.41 (s, 1H), 8.72 (s, 1H), 9.04-9.07 (m, 2H), 10.78 (s, 1H). One proton was not apparent by ¹H NMR. |
| P-208 | A57 | C48 | 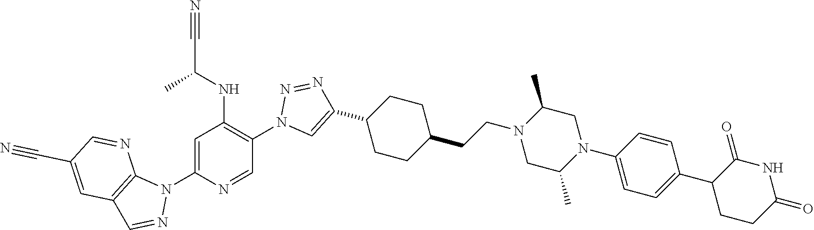 | 40% yield as an off-white solid. LCMS method 6: Retention time: 1.978 min, 99.0% purity at 215 nm, [M + H]⁺ = 766.5 ¹H NMR (400 MHz, DMSO-d6, 80 C): δ ppm 0.96-1.02 (m, 2H), 1.23-1.36 (m, 6H), 1.46-1.68 (m, 8H), 1.93-1.96 (m, 2H), 2.11- 2.22 (m, 4H), 2.62-2.68 (m, 1H), 2.78-2.83 (m, 1H), 2.95-3.05 (m, 1H), 3.10-3.18 (m, 1H) 3.33- 3.40 (m, 4H), 3.82-3.89 (m, 2H), 4.94-4.98 (m, 1H), 7.06-7.21 (m, 5H), 7.74 (s, 1H), 8.33 (s, 1H), 8.46 (s, 1H), 8.67 (s, 1H), 8.99-9.02 (m, 2H), 10.50 (s, 1H). Three protons were not apparent by ¹H NMR. |
| P-209 | A57 | C25 | 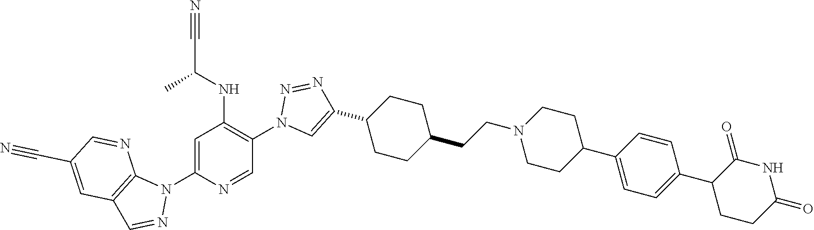 | 36% yield as an off-white solid. LCMS method 6: Retention time: 1.966 min, 99.9% purity at 215 nm, [M + H]⁺ = 737.3 ¹H NMR (400 MHz, DMSO-d6): δ ppm 1.15-1.22 (m, 2H), 1.38- 1.67 (m, 8H), 1.85-1.91 (m, 4H), 2.00-2.06 (m, 3H), 2.15-2.18 (m, 3H), 2.68-2.84 (m, 2H), 3.04- 3.18 (m, 4H), 3.32-3.38 (m, 1H), 3.82-3.87 (m, 1H), 5.01-5.05 (m, 1H), 7.19-7.23 (m, 5H), 7.68 (s, 1H), 8.38 (s, 1H), 8.42 (s, 1H), 8.73 (s, 1H), 9.07-9.09 (m, 2H), 10.9 (s, 1H). Three protons were not apparent by ¹H NMR. |
| P-210 | A57 | C73i | 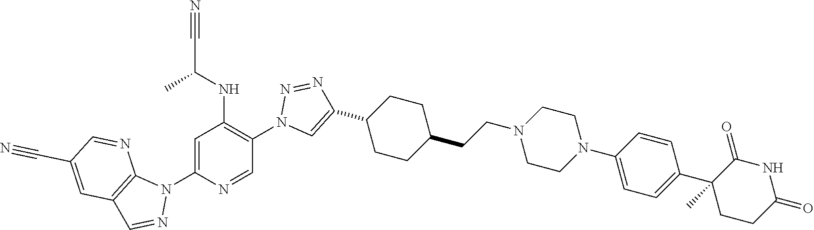 | 15% yield as an off-white solid. LCMS method 7: Retention time: 1.758 min, 97.6% purity at 215 nm, [M + H]⁺ = 752.4 ¹H NMR (400 MHz, DMSO-d6): δ ppm 1.15-1.22 (m, 2H), 1.40 (s, 3H), 1.46-1.55 (m, 2H), 1.60- 1.66 (m, 5H), 1.87-1.90 (m, 2H), 2.04-2.18 (m, 4H), 2.43-2.47 (m, 2H), 2.75-2.80 (m, 1H), 2.95-3.01 (m, 2H), 3.12-3.26 (m, 4H), 3.60-3.63 (m, 2H), 3.84- 3.87 (m, 2H), 5.01-5.05 (m, 1H), 7.02 (d, J = 8.8 Hz, 2H), 7.18 (d, J = 8.8 Hz, 2H), 7.19-7.21 (m, 1H), 7.68 (s, 1H), 8.37 (s, 1H), 8.41 (s, 1H), 8.73 (s, 1H), 9.06- 9.08 (m, 2H), 10.88 (s, 1H). One proton was not apparent by ¹H NMR. |
| P-218 | A-83 | C12 | 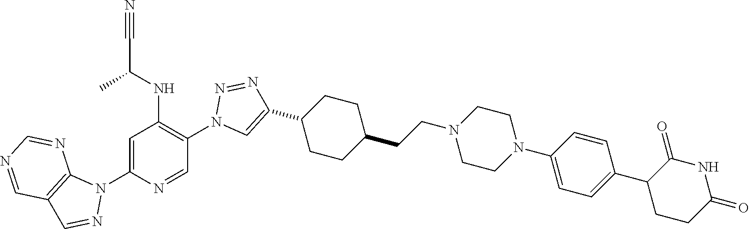 | 4.3% yield as an off-white solid. LCMS method 7: Retention time: 1.390 min, 96.8% purity at 215 nm, [M + H]⁺ = 714.3 ¹H NMR (400 MHz, DMSO-d6): δ ppm 1.16-1.23 (m, 2H), 1.39- 1.59 (m, 3H), 1.60-1.66 (m, 5H), 1.87-1.90 (m, 2H), 1.96-2.03 (m, 1H), 2.14-2.17 (m, 3H), 2.75- 2.82 (m, 1H), 2.91-3.01 (m, 3H), 3.10-3.23 (m, 5H), 3.60-3.62 (m, 2H), 3.77-3.85 (m, 3H), 5.01-5.04 (m, 1H), 6.98 (d, J = 8.8 Hz, 2H), 7.12 (d, J = 8.8 Hz, 2H), 7.18 (d, J = 8.4 Hz, 1H), 7.71 (s, 1H), 8.36 (s, 1H), 8.40 (s, 1H), 8.74 (s, 1H), 9.18 (s, 1H), 9.51 ( s, 1H), 10.79 (s, 1H). |
| P-221 | A-66 | C-32 | 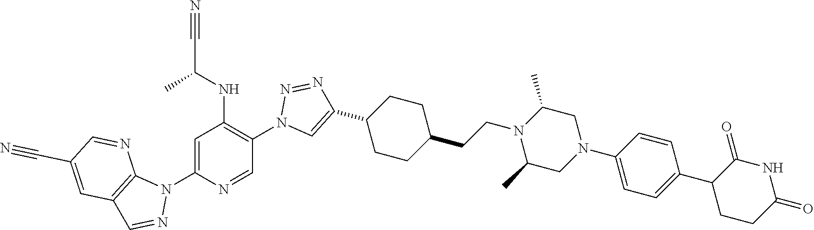 | 5.6% yield as an off-white solid. LCMS method 7: Retention time: 1.533 min, 99.7% purity at 215 nm, [M + H]⁺ = 766.2 ¹H NMR (400 MHz, DMSO-d6): δ ppm 1.20-1.32 (m, 2H), 1.36- 1.39 (m, 6H), 1.44-1.71 (m, 8H), 1.90-2.02 (m, 4H), 2.14-2.17 (m, 2H), 2.71-2.82 (m, 1H), 3.61- 3.66 (m, 2H), 3.73-3.79 (m, 2H), 3.85-3.92 (m, 1H), 4.98-5.02 (m, 1H), 6.97 (d, J = 8.8 Hz, 2H), 7.08-7.21 (m, 3H), 7.67 (s, 1H), 8.37 (s, 1H), 8.41 (s, 1H), 8.71 (s, 1H), 9.04-9.07 (m, 2H), 10.77 (s, 1H) Six protons were not apparent by ¹H NMR. |
| P-225 | A-83 | C-73 | 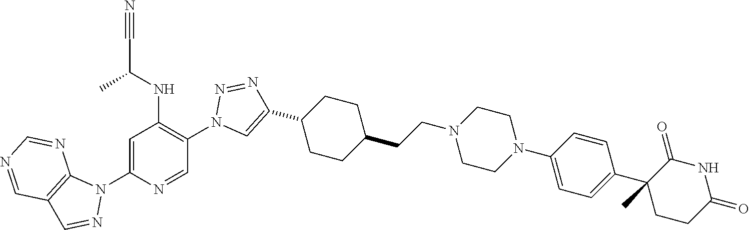 | 44.2% yield as an off-white solid. LCMS method 7: Retention time: 1.422 min, 99.8% purity at 215 nm, [M + H]⁺ = 728.4 ¹H NMR (400 MHz, DMSO-d6): δ ppm 1.15-1.25 (m, 2H), 1.38- 1.41 (m, 4H), 1.47-1.53 (m, 2H), 1.61-1.66 (m, 5H), 1.86-1.89 (m, 2H), 2.06-2.17 (m, 4H), 2.42- 2.48 (m, 1H), 2.73-2.79 (m, 1H), 2.96-2.99 (m, 2H), 3.10-3.25 (m, 4H), 3.59-3.62 (m, 2H), 3.83-3.86 (m, 2H), 5.00-5.05 (m, 1H), 7.01 (d, J = 8.8 Hz, 2H), 7.16-7.20 (m, 3H), 7.71 (s, 1H), 8.36 (s, 1H), 8.40 (s, 1H), 8.74 (s, 1H), 9.18 (s, 1H), 9.51 (s, 1H), 10.87 (s, 1H) One proton was not apparent by ¹H NMR. |
| P-228 | A-86 | C-73 | 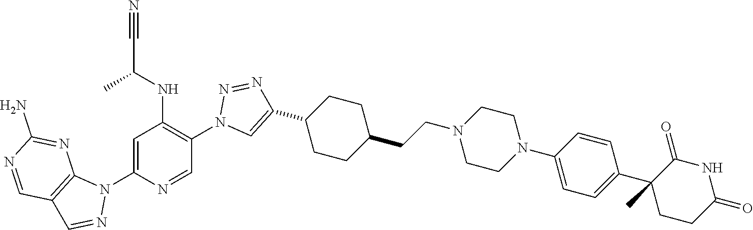 | 6.5% yield as an off-white solid. LCMS method 10: Retention time: 1.966 min, 99.1% purity at 215 nm, [M + H]⁺ = 743.4 ¹H NMR (400 MHz, DMSO-d6): δ ppm 1.15-1.26 (m, 2H), 1.36- 1.41 (m, 4H), 1.42-1.59 (m, 2H), 1.61-1.72 (m, 5H), 1.86-1.90 (m, 2H), 2.00-2.20 (m, 4H), 2.70- 2.83 (m, 1H), 2.90-3.12 (m, 2H), 3.15-3.19 (m, 4H), 3.83-3.89 (m, 2H), 4.94-5.02 (m, 1H), 6.97-7.09 (m, 3H), 7.16-7.22 (m, 4H), 7.91 (s, 1H), 8.27-8.32 (m, 3H), 8.92 (s, 1H), 10.87 (s, 1H) Four protons were not apparent by ¹H NMR. |
| P-229 | A-57 | C-46 | 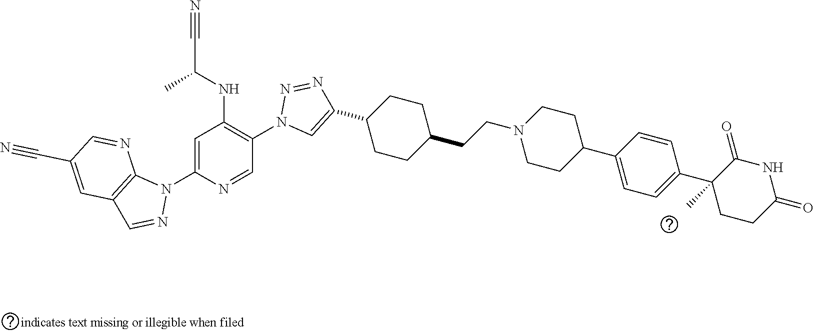 | 7.4% yield as an off-white solid. LCMS method 13: Retention time: 1.470 min, 97.7% purity at 215 nm, [M + H]⁺ = 755.4 ¹H NMR (500 MHz, DMSO-d6) δ 9.10-9.02 (m, 2H), 8.72 (s, 1H), 8.41 (s, 1H), 8.35 (s, 1H), 7.67 (s, 1H), 7.42-7.27 (m, 4H), 7.23-7.17 (m, 1H), 5.01 (t, J = 7.5 Hz, 1H), 2.77-2.62 (m, 2H), 2.29 (br d, J = 3.7 Hz, 2H), 2.13 (br d, J = 10.3 Hz, 2H), 1.95-1.83 (m, 4H), 1.76 (br d, J = 7.6 Hz, 2H), 1.71-1.64 (m, 1H), 1.61 (d, J = 6.9 Hz, 4H), 1.54-1.33 (m, 5H), 1.13 (br d, J = 12.6 Hz, 2H). ¹⁹F NMR (471 MHz, DMSO-d6) δ −73.41 (s, 1F). |
| P-230 | A-87 | C-73 | 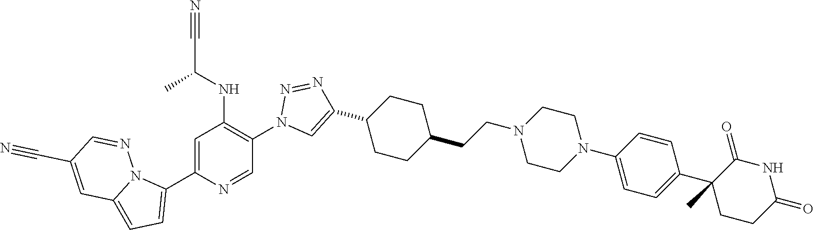 | 9.8% yield as a yellow solid. LCMS method 7: Retention time: 2.017 min, 97.7% purity at 215 nm, [M + H]⁺ = 751.4 ¹H NMR (400 MHz, DMSO-d6): δ ppm 1.16-1.23 (m, 2H), 1.40 (s, 3H), 1.48-1.51 (m, 2H), 1.63- 1.67 (m, 5H), 1.87-1.89 (m, 2H), 2.07-2.16 (m, 4H), 2.71- 2.77 (m, 1H), 2.97-3.00 (m, 2H), 3.12-3.26 (m, 4H), 3.59-3.62 (m, 2H), 3.83-3.87 (m, 2H), 4.89- 4.94 (m, 1H), 7.01 (d, J = 8.8 Hz, 2H), 7.07-7.09 (m, 1H), 7.15- 7.21 (m, 3H), 7.85 (d, J = 4.8 Hz, 1H), 8.37 (s, 1H), 8.40 (s, 1H), 8.48 (s, 1H), 8.68 (d, J = 2.0 Hz, 1H), 8.89 (d, J = 2.0 Hz, 1H), 10.87 (s, 1H) Three protons were not apparent by ¹H NMR. |
| P-231 | A-86 | C-12 | 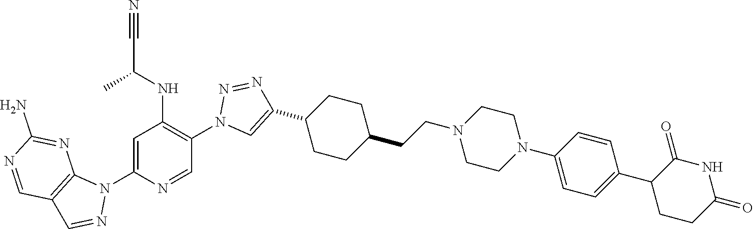 | 5.4% yield as an off-white solid. LCMS method 8: Retention time: 1.854 min, 96.8% purity at 215 nm, [M + H]⁺ = 729.3 ¹H NMR (400 MHz, DMSO-d6): δ ppm 1.15-1.28 (m, 2H), 1.32- 1.60 (m, 3H), 1.62-1.67 (m, 5H), 1.84-1.90 (m, 2H), 1.93-2.04 (m, 1H), 2.11-2.23 (m, 3H), 2.62- 2.78 (m, 2H), 2.92-3.03 (m, 2H), 3.12-3.40 (m, 4H), 3.61-3.78 (m, 2H), 3.82-3.90 (m, 3H), 4.91-5.02 (m, 1H), 6.98-7.00 (m, 2H), 7.07-7.24 (m, 5H), 7.92 (s, 1H), 8.29-8.34 (m, 3H), 8.93 (s, 1H), 10.80 (s, 1H) One proton was not apparent by ¹H NMR. |
| P-232 | A-88 | C-75 | 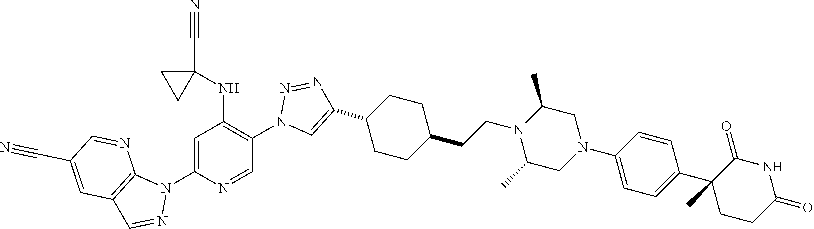 | 42.1% yield as an off-white solid. LCMS method 8: Retention time: 2.270 min, 98.9% purity at 215 nm, [M + H]⁺ = 792.5 ¹H NMR (400 MHz, DMSO-d6): δ ppm 1.14-1.40 (m, 2H), 1.31- 1.43 (m, 11H), 1.44-1.60 (m, 4H), 1.61-1.70 (m, 1H), 1.71-1.78 (m, 2H), 1.91-1.93 (m, 2H), 2.02- 2.10 (m, 2H), 2.11-2.20 (m, 2H), 2.71-2.88 (m, 2H), 3.01-3.12 (m, 1H), 3.60-3.70 (m, 2H), 3.74-3.81 (m, 1H), 3.84-3.92 (m, 1H), 7.01 (d, J = 8.8 Hz, 2H), 7.17 (d, J = 8.8 Hz, 2H), 7.75 (s, 1H), 8.00 (s, 1H), 8.31 (s, 1H), 8.40 (s, 1H), 8.75 (s, 1H), 9.07 (d, J = 2.00 Hz, 1H), 9.12 (d, J = 2.00 Hz, 1H), 10.87 (s, 1H). Four protons were not apparent by ¹H NMR. |
| P-233 | A-57 | C-76 | 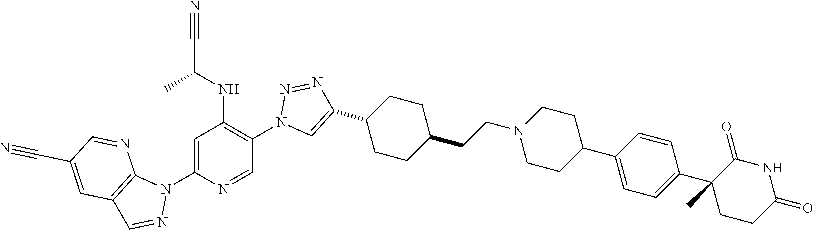 | 25.0% yield as a pale brown solid. LCMS method 7: Retention time: 1.809 min, 97.4% purity at 215 nm, [M + H]⁺ = 751.4 ¹H NMR (400 MHz, DMSO-d6): δ ppm 1.13-1.18 (m, 2H), 1.38- 1.55 (m, 7H), 1.59-1.62 (m, 4H), 1.86-1.91 (m, 4H), 2.04-2.15 (m, 6H), 2.32-2.40 (m, 2H), 2.72- 2.77 (m, 2H), 5.00-5.04 (m, 1H), 7.19-7.25 (m, 5H), 7.67 (s, 1H), 8.36 (s, 1H), 8.41 (s, 1H), 8.72 (s, 1H), 9.06-9.07 (m, 2H), 10.90 (s, 1H). Six protons were not apparent by ¹H NMR. |
| P-234 | A-88 | C-73 | 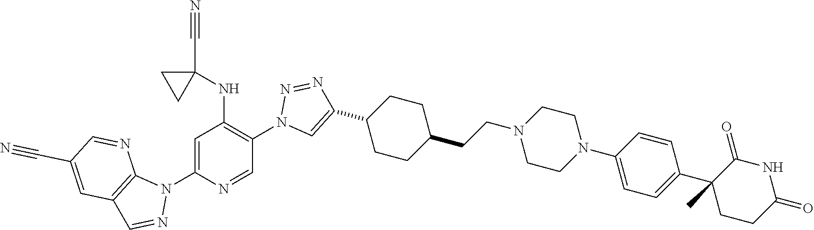 | 36.4% yield as a white solid. LCMS method 7: Retention time: 1.429 min, 92.6% purity at 215 nm, [M + H]⁺ = 764.2 ¹H NMR (400 MHz, DMSO-d6): δ ppm 1.14-1.28 (m, 2H), 1.39- 1.56 (m, 7H), 1.64-1.76 (m, 4H), 1.85-1.92 (m, 2H), 2.08-2.17 (m, 4H), 2.33-2.38 (m, 1H), 2.43- 2.48 (m, 1H), 2.72-2.80 (m, 1H), 2.96-3.03 (m, 2H), 3.15-3.28 (m, 2H), 3.60-3.62 (m, 2H), 3.84-3.87 (m, 2H), 7.02 (d, J = 8.8 Hz, 2H), 7.18 (d, J = 8.8 Hz, 2H), 7.75 (s, 1H), 8.00 (s, 1H), 8.30 (s, 1H), 8.40 (s, 1H), 8.75 (s, 1H), 9.08-9.12 (m, 2H), 10.88 (s, 1H). Three protons were not apparent by ¹H NMR. |
| P-235 | A-57 | C-77 | 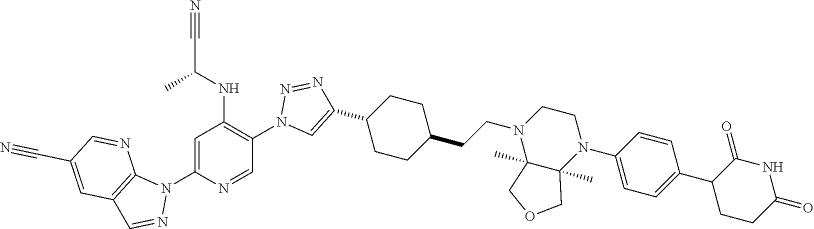 | 22% yield as an off-white solid. LCMS method 13: Retention time: 1.330 min, 96.1% purity at 215 nm, [M + H]⁺ = 780.2 ¹H NMR (500 MHz, DMSO-d6) δ 10.75 (s, 1H), 9.05 (dd, J = 10.0, 1.9 Hz, 4H), 8.75-8.69 (m, 2H), 8.50-8.38 (m, 2H), 8.38-8.33 (m, 2H), 7.74-7.62 (m, 3H), 7.19 (br d, J = 8.0 Hz, 3H), 7.04 (br d, J = 8.6 Hz, 5H), 6.97-6.82 (m, 5H), 5.10-4.91 (m, 2H), 4.42 (br s, 2H), 3.97-3.91 (m, 1H), 3.82 (br dd, J = 9.5, 4.0 Hz, 1H), 3.79- 3.62 (m, 2H), 3.62-3.47 (m, 1H), 3.44-3.32 (m, 1H), 3.17 (d, J = 5.2 Hz, 1H), 3.10-2.89 (m, 1H), 2.86-2.68 (m, 2H), 2.68-2.57 (m, 1H), 2.50-2.40 (m, 2H), 2.23- 2.07 (m, 4H), 2.07-1.95 (m, 1H), 1.95-1.76 (m, 2H), 1.61 (d, J = 6.9 Hz, 5H), 1.45 (br d, J = 7.3 Hz, 4H), 1.41-1.29 (m, 2H), 1.14 (br d, J = 5.5 Hz, 2H). |
| P-236 | A-57 | C-78 | 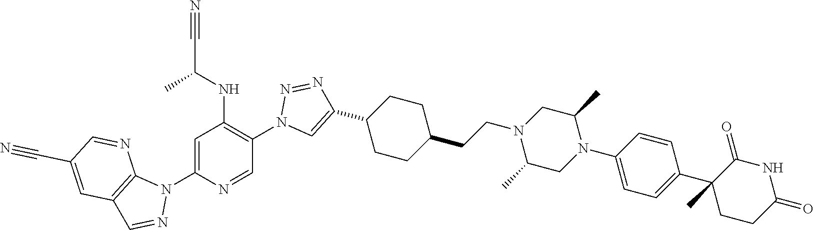 | 12.0% yield as a brown gum. LCMS method 8: Retention time: 2.370 min, 96.7% purity at 215 nm, [M + H]⁺ = 780.4 ¹H NMR (400 MHz, DMSO-d6, 80 oC): δ ppm 0.94-1.04 (m, 2H), 1.15-1.30 (m, 3H), 1.35 (d, J = 6.40 Hz, 3H), 1.48-1.62 (m, 6H), 1.65-1.80 (m, 5H), 1.93-1.96 (m, 2H), 2.08-2.22 (m, 4H), 2.33- 2.38 (m, 2H), 2.79-2.88 (m, 1H), 2.92-3.21 (m, 2H), 3.33-3.65 (m, 5H), 4.94-5.00 (m, 1H), 7.07- 7.28 (m, 5H), 7.74 (s, 1H), 8.32 (s, 1H), 8.46 (s, 1H), 8.66 (s, 1H), 8.98 (d, J = 2.0 Hz, 1H), 9.02 (d, J = 2.0 Hz, 1H), 10.53 (s, 1H). One proton was not apparent by ¹H NMR. |
| P-237 | A-57 | C77i | 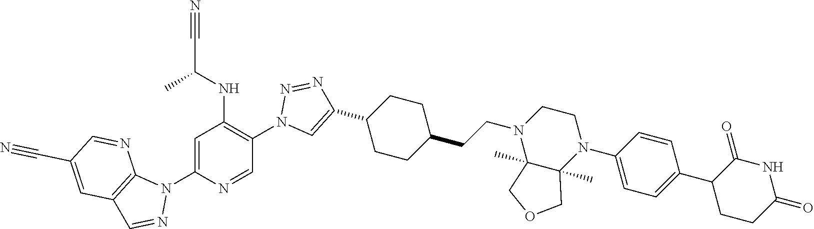 | 17% yield as an off-white solid. LCMS method 13: Retention time: 1.360 min, 99.8% purity at 215 nm, [M + H]⁺ = 780.0 ¹H NMR (500 MHz, DMSO-d6) δ 10.75 (s, 1H), 9.20-8.88 (m, 2H), 8.70 (s, 1H), 8.39 (s, 1H), 8.34 (s, 1H), 7.66 (s, 1H), 7.18 (d, J = 8.0 Hz, 1H), 7.03 (d, J = 8.6 Hz, 2H), 6.89 (br d, J = 8.5 Hz, 2H), 4.99 (br t, J = 7.2 Hz, 1H), 4.42 (br s, 1H), 3.93 (br d, J = 8.6 Hz, 1H), 3.82 (dd, J = 9.0, 3.6 Hz, 1H), 3.76-3.69 (m, 2H), 3.09-2.93 (m, 2H), 2.83-2.67 (m, 3H), 2.67- 2.58 (m, 1H), 2.48-2.41 (m, 2H), 2.13 (br d, J = 10.7 Hz, 5H), 2.07- 1.96 (m, 1H), 1.60 (d, J-6.9 Hz, 3H), 1.55-1.40 (m, 5H), 1.40- 1.32 (m, 1H), 1.21-1.07 (m, 2H). |
| P-238 | A-57 | C-75 | 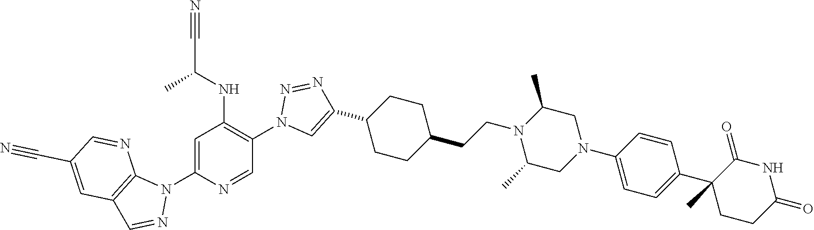 | 17.4% yield as a yellow solid. LCMS method 11: Retention time: 2.326 min, 91.1% purity at 215 nm, [M + H]⁺ = 780.4 ¹H NMR (400 MHz, DMSO-d6): δ ppm 1.13-1.26 (m, 2H), 1.36- 1.42 (m, 8H), 1.45-1.69 (m, 7H), 1.91-1.96 (m, 2H), 2.04-2.10 (m, 2H), 2.15-2.21 (m, 2H), 2.74- 2.87 (m, 2H), 3.02-3.19 (m, 2H), 3.32-3.40 (m, 2H), 5.00- 5.07 (m, 1H), 7.01 (d, J = 8.8 Hz, 2H), 7.16-7.21 (m, 3H), 7.68 (s, 1H), 8.38 (s, 1H), 8.42 (s, 1H), 8.73 (s, 1H), 9.06-9.08 (m, 2H), 10.88 (s, 1H) Seven protons were not apparent by ¹H NMR. |
| P-239 | A-57 | C-79 | 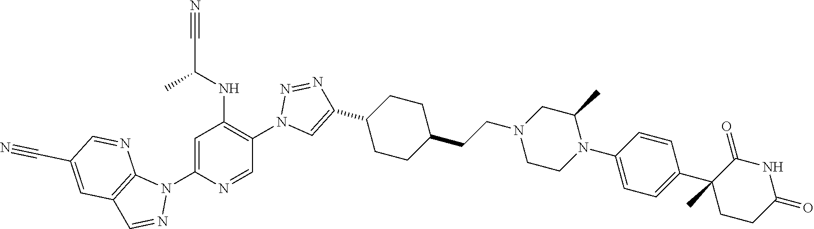 | 11.0% yield as an off-white solid. LCMS method 11: Retention time: 2.326 min, 91.1% purity at 215 nm, [M + H]⁺ = 766.4 ¹H NMR (400 MHz, DMSO-d6 + TFA): δ ppm 0.95 (m, 1H), 1.09- 1.21 (m, 4H), 1.46-1.57 (m, 6H), 1.61-1.69 (m, 5H), 1.87-1.90 (m, 2H), 2.05-2.18 (m, 4H), 2.77- 2.80 (m, 1H), 3.21-3.27 (m, 4H), 3.51-3.63 (m, 3H), 5.00- 5.06 (m, 1H), 6.95 (s, 1H), 7.17- 7.31 (m, 3H), 7.80 (s, 1H), 8.34 (s, 1H), 8.45 (s, 1H), 8.73 (s, 1H), 9.03-9.07 (m, 2H). Four protons were not apparent by ¹H NMR. |
| P-240 | A-88 | C-78 | 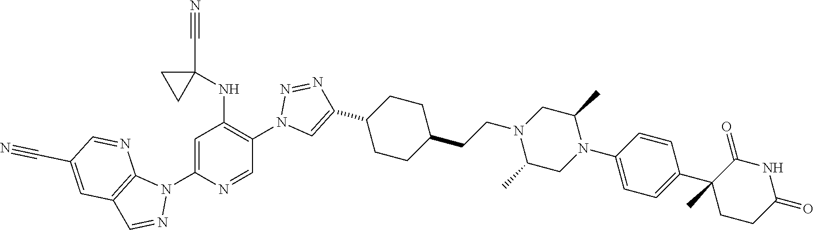 | 26.0% yield as an off-white solid. LCMS method 11: Retention time: 2.330 min, 95.2% purity at 215 nm, [M + H]⁺ = 792.4 ¹H NMR (400 MHz, DMSO-d6): δ ppm 0.94 and 1.12 (d, J = 6.0 Hz, 3H), 1.19-1.33 (m, 5H), 1.38- 1.52 (m, 9H), 1.61-1.80 (m, 5H), 1.89-1.92 (m, 2H), 2.04-2.18 (m, 3H), 2.74-2.80 (m, 1H), 2.88-3.00 (m, 1H), 3.08-3.22 (m, 2H), 3.25-3.51 (m, 3H), 3.52- 3.68 (m, 1H), 6.94-6.96 (m, 1H), 7.13-7.28 (m, 3H), 7.75 (s, 1H), 8.01 (d, J = 5.2 Hz, 1H), 8.31 (s, 1H), 8.43 (s, 1H), 8.75 (s, 1H), 9.08 (d, J = 2.0 Hz, 1H), 9.12 (d, J = 2.0 Hz, 1H), 10.87 and 10.93 (s, 1H). Two protons were not apparent by ¹H NMR. |
| P-241 | A-57 | C-80 | 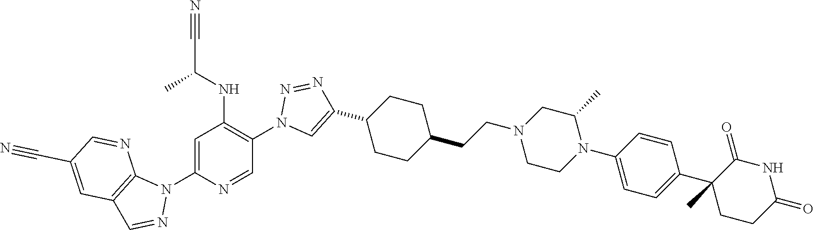 | 12.7% yield as an off-white solid. LCMS method 7: Retention time: 1.810 min, 96.7% purity at 215 nm, [M + H]⁺ = 766.4 ¹H NMR (400 MHz, DMSO-d6): δ ppm 0.92 and 1.10 (d, J = 6.0 Hz, 3H), 1.20-1.28 (m, 3H), 1.40- 1.58 (m, 6H), 1.61-1.68 (m, 5H), 1.88-1.94 (m, 2H), 2.06-2.18 (m, 4H), 2.71-2.79 (m, 1H), 3.02-3.18 (m, 3H), 3.25-3.31 (m, 4H), 4.38-4.42 (m, 1H), 4.98- 5.06 (m, 1H), 6.96-7.29 (m, 5H), 7.68 (s, 1H), 8.37 (s, 1H), 8.41 (s, 1H), 8.77 (s, 1H), 9.06-9.08 (m, 2H), 10.87 and 10.93 (s, 1H). Two protons were not apparent by ¹H NMR. |
| P-243 | A-57 | C-81 | 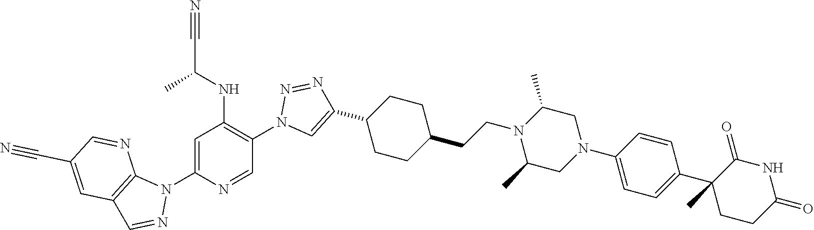 | 6.0% yield as an off-white solid. LCMS method 7: Retention time: 2.312 min, 96.5% purity at 215 nm, [M + H]⁺ = 780.4 ¹H NMR (400 MHz, DMSO-d6): δ ppm 1.14-1.28 (m, 2H), 1.35- 1.40 (m, 9H), 1.46-1.68 (m, 7H), 1.90-1.94 (m, 2H), 2.05-2.18 (m, 4H), 2.31-2.36 (m, 1H), 2.42- 2.48 (m, 1H), 2.73-2.86 (m, 2H), 3.01-3.11 (m, 1H), 3.15-3.18 (m, 1H), 3.31-3.42 (m, 2H), 3.76-3.79 (m, 1H), 3.85-3.93 (m, 1H), 5.00-5.04 (m, 1H), 7.01 (d, J = 8.8 Hz, 2H), 7.12-7.25 (m, 3H), 7.68 (s, 1H), 8.38 (s, 1H), 8.42 (s, 1H), 8.72 (s, 1H), 9.06- 9.08 (m, 2H), 10.87 (s, 1H). Two protons were not apparent by ¹H NMR. |
| P-244 | A-57 | C-82 | 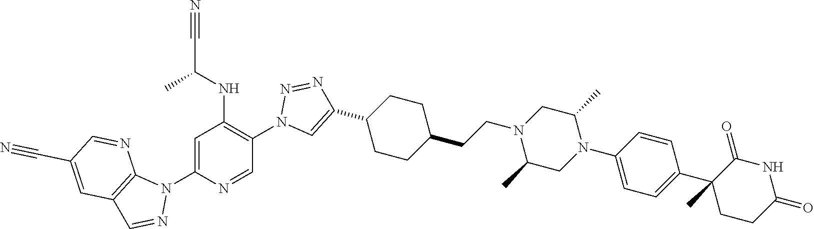 | 6.4% yield as a pale yellow solid. LCMS method 7: Retention time: 1.850 min, 96.4% purity at 215 nm, [M + H]⁺ = 780.4 ¹H NMR (400 MHz, DMSO-d6 -D20 exchange): δ ppm 0.92 (t, J = 6.0 Hz, 3H), 1.02 (t, J = 6.0 Hz, 3H), 1.10-1.20 (m, 2H), 1.38 - 1.49 (m, 8H), 1.60 (d, J = 7.20 Hz, 3H), 1.83-1.86 (m, 2H), 2.01- 2.11 (m, 4H), 2.29-2.33 (m, 1H), 2.68-2.75 (m, 2H), 2.91-3.06 (m, 3H), 3.08-3.15 (m, 1H), 3.53- 3.55 (m, 1H), 4.91-4.93 (m, 1H), 6.98 (d, J = 8.8 Hz, 2H), 7.14 (d, J = 8.4 Hz, 2H), 7.69 (s, 1H), 8.27 (s, 1H), 8.36 (s, 1H), 8.66 (s, 1H), 8.95 (d, J = 2.0 Hz, 1H), 8.99 (d, J = 2.0 Hz, 1H). Three protons were not apparent by ¹H NMR. |

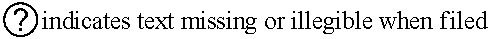

General Procedure X-12. The scheme shown below for the synthesis of P-103 is provided as a representative synthesis for General Procedure X-12.

### Example S52. 6-(5-cyanopyrazolo[3,4-b]pyridin-1-yl)-4-(cyclopropylamino)-N-[4-[[1-[4-(2,6-dioxo-3-piperidyl)phenyl]-4-piperidyl]methyl-methyl-amino] cyclohexyl]pyridine-3-carboxamide (P-103)

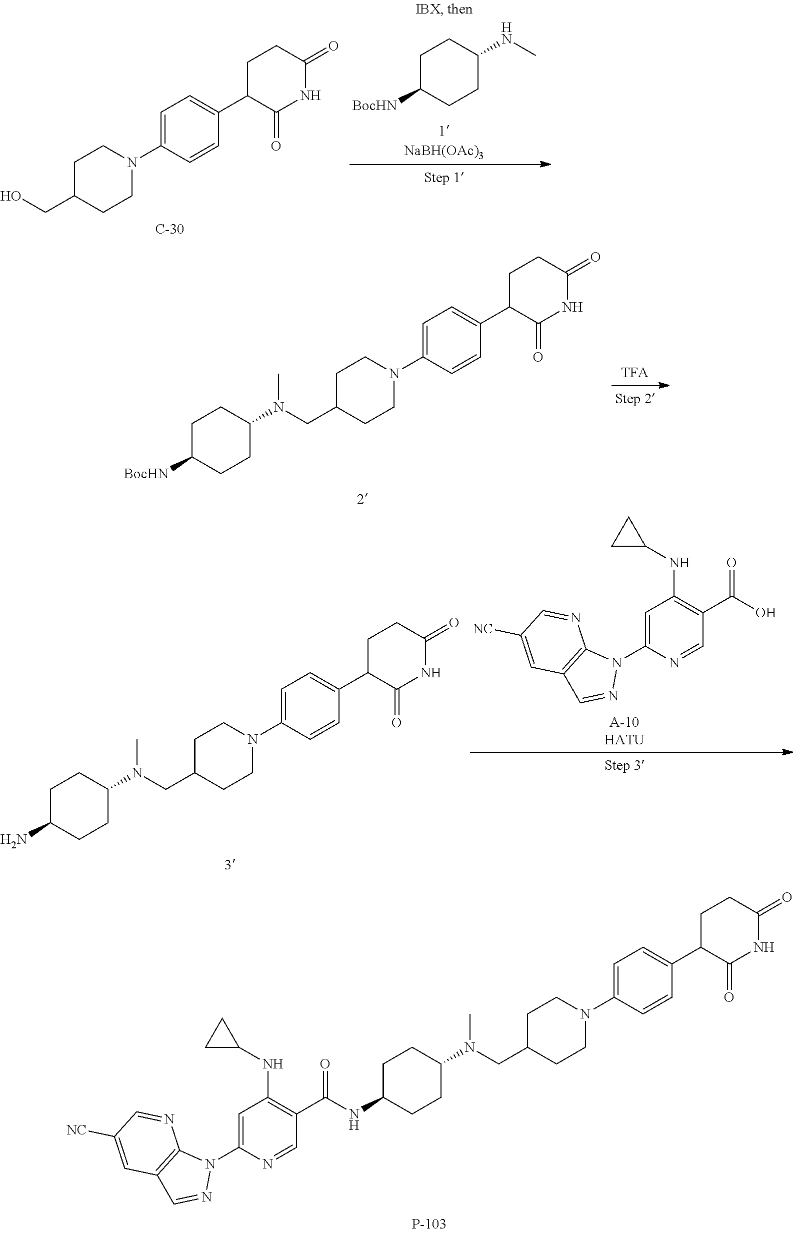

Step 1. Preparation of tert-butyl N-[4-[[1-[4-(2,6-dioxo-3-piperidyl)phenyl]-4-piperidyl]methyl-methyl-amino]cyclohexyl]carbamate (2): To a solution of 3-[4-[4-(hydroxymethyl)-1-piperidyl]phenyl]piperidine-2,6-dione C-38 (150 mg, 0.44 mmol, 1 eq.) in dry DMSO (1.5 mL, 0.18 M) was added IBX (148.76 mg, 0.53 mmol, 1.2 eq.). The resulting mixture was stirred at room temperature overnight. LCMS after an overnight stirring showed complete conversion to the desired aldehyde. To the reaction mixture was then added tert-butyl N-[4-(methylamino)cyclohexyl]carbamate 1′ (121.3 mg, 0.53 mmol, 1.2 eq.), DCE (2.5 mL, 0.18 M) and DIPEA (0.77 mL, 4.43 mmol, 10 eq.). The mixture was stirred at RT for 10 minutes, then sodium triacetoxyborohydride (121.97 mg, 0.58 mmol, 1.3 eq.) was added. After 1 h, LCMS showed full conversion. The DCE was evaporated and the reaction mixture was directly loaded for reverse phase FC purification (50 g C18 column, liquid deposit (DMSO), 5% MeCN/0.1% HCOOH over 4 CV, then 5 to 95% MeCN/0.1% HCOOH over 20 CV, product came out at 50% MeCN). Pure fractions were combined and concentrated, affording 2′ (52 mg, 23% yield) as a white solid.

LCMS method 1: 97.3% purity at 215 nm, [M+H]⁺=513.4.

¹H NMR (400 MHz, DMSO-d6) δ ppm 1.09-1.22 (m, 4H), 1.23-1.32 (m, 2H), 1.37 (s, 9H), 1.50-1.62 (m, 1H), 1.66-1.87 (m, 6H), 1.96-2.05 (m, 1H), 2.08-2.17 (m, 1H), 2.20-2.40 (m, 1H), 2.41-2.48 (m, 1H), 2.57-2.66 (m, 3H), 3.04-3.16 (m, 1H), 3.64 (br d, J=12.2 Hz, 2H), 3.71 (br dd, J=11.0, 4.9 Hz, 1H), 6.68 (br d, J=8.8 Hz, 1H), 6.87 (br d, J=8.6 Hz, 2H), 7.02 (d, J=8.6 Hz, 2H), 10.76 (s, 1H). One proton was missing on the spectrum.

Step 2. Preparation of 3-[4-[4-[[(4-aminocyclohexyl)-methyl-amino]methyl]-1-piperidyl]phenyl]piperidine-2,6-dione (3′): To of tert-butyl N-[4-[[1-[4-(2,6-dioxo-3-piperidyl)phenyl]-4-piperidyl]methyl-methyl-amino]cyclohexyl]carbamate 2′ (30 mg, 0.06 mmol, 1 eq.) in DCM (3.9 mL, 0.02 M) was added TFA (0.09 mL, 1.17 mmol, 20 eq.). The reaction mixture was stirred at rt. After an overnight period, LCMS showed complete conversion. The solvent was removed under reduced pressure and the residue was co-evaporated with toluene (3×) and MeCN (2×). The residue was dried under high vacuum, affording 3′ (30 mg, quantitative yield) as an off-white solid that was used as is for the next step.

LCMS method 3: 99.9% purity at 215 nm, [M+H]⁺=413.4.

Step 3. Preparation of 6-(5-cyanopyrazolo[3,4-b]pyridin-1-yl)-4-(cyclopropylamino)-N-[4-[[1-[4-(2,6-dioxo-3-piperidyl)phenyl]-4-piperidyl]methyl-methyl-amino]cyclohexyl]pyridine-3-carboxamide (P-103): To a solution of 3-[4-[4-[[(4-aminocyclohexyl)-methyl-amino]methyl]-1-piperidyl]phenyl]piperidine-2,6-dione 3′ (85.01 mg, 0.21 mmol, 1.2 eq.) and 6-(5-cyanopyrazolo[3,4-b]pyridin-1-yl)-4-(cyclopropylamino)pyridine-3-carboxylic acid A-10 (55 mg, 0.17 mmol, 1 eq.) in DMF (0.98 mL, 0.18 M) were added DIPEA (0.3 mL, 1.72 mmol, 10 eq.) and HATU (78.35 mg, 0.21 mmol, 1.2 eq.). The solution was then stirred at room temperature. After 18 h, LCMS showed complete conversion. The residue was directly purified by reverse phase flash chromatography (50 g C18 column, liquid deposit (DMSO), elution: 5% MeCN/0.1% HCOOH over 3 CV, then 5 to 40% MeCN/0.1% HCOOH over 20 CV, product came out at 25 MeCN). Fractions were combined, concentrated and lyophilized, affording P-103 (42.1 mg, 34% yield) as an off-white solid.

LCMS method 2: retention time 98.5% purity at 215 nm [M+H]⁺=715.4.

¹H NMR (400 MHz, DMSO-d6) δ ppm 0.53-0.59 (m, 2H), 0.83-0.88 (m, 2H), 1.12-1.22 (m, 2H), 1.28-1.42 (m, 4H), 1.50-1.59 (m, 1H), 1.73-1.84 (m, 4H), 1.88-1.95 (m, 2H), 1.97-2.04 (m, 1H), 2.07-2.17 (m, 1H), 2.21 (s, 3H), 2.24-2.30 (m, 2H), 2.32-2.39 (m, 1H), 2.41-2.48 (m, 1H), 2.55-2.67 (m, 4H), 3.63-3.74 (m, 4H), 6.88 (d, J=8.6 Hz, 2H), 7.03 (d, J=8.6 Hz, 2H), 7.67 (s, 1H), 8.38 (d, J=7.8 Hz, 1H), 8.54 (s, 1H), 8.59 (s, 1H), 8.65 (s, 1H), 9.00-9.04 (m, 1H), 9.04-9.08 (m, 1H), 10.77 (s, 1H).

Table 41 summarizes the compounds prepared using General Procedure X-12.

TABLE 41
Final Compounds Prepared via General Procedure X-12

| Com- | | | | |
|---|---|---|---|---|
| pound | TMB | CBM | | |
| No. | Portion | Portion | Structure | Characterization |
| P-88 | A10 | C38 | 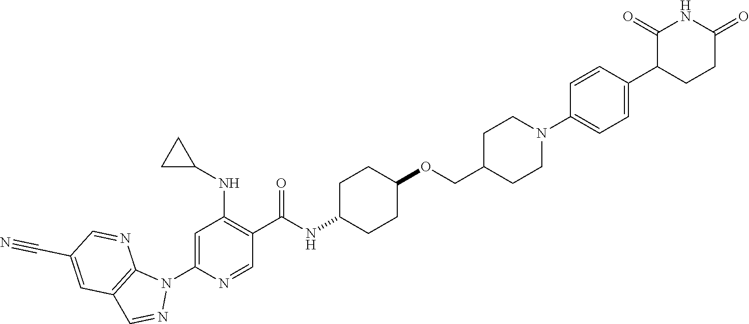 Perform mesylate displacement of tert-butyl N-[cis- 4-(methanesulfonyloxy)cyclohexyl] carbamate in step 1 | 23% yield as a light yellow solid. LCMS method 2: Retention time: 2.765 min, 99.9% purity at 215 nm [M + H]⁺ = 702.4, [M + 2H]²⁺ = 351.8 ¹H NMR (400 MHz, DMSO-d6) δ ppm 0.52-0.60 (m, 2 H), 0.80-0.90 (m, 2 H), 1.12-1.46 (m, 8 H), 1.71-1.80 (m, 2 H), 1.83-1.93 (m, 2 H), 1.95-2.20 (m, 4 H), 2.41- 2.48 (m, 1 H), 2.54-2.69 (m, 5 H), 3.15-3.31 (m, 2 H), 3.62-3.77 (m, 3 H), 6.88 (d, J = 8.6 Hz, 2 H), 7.03 (d, J = 8.6 Hz, 2 H), 7.67 (s, 1 H), 8.39 (br d, J = 7.8 Hz, 1 H), 8.52 (s, 1 H), 8.59 (s, 1 H), 8.66 (s, 1 H), 9.02 (d, J = 1.7 Hz, 1 H), 9.06 (d, J = 2.0 Hz, 1 H), 10.77 (s, 1 H). |
| P-102 | A-10 | C-38 | 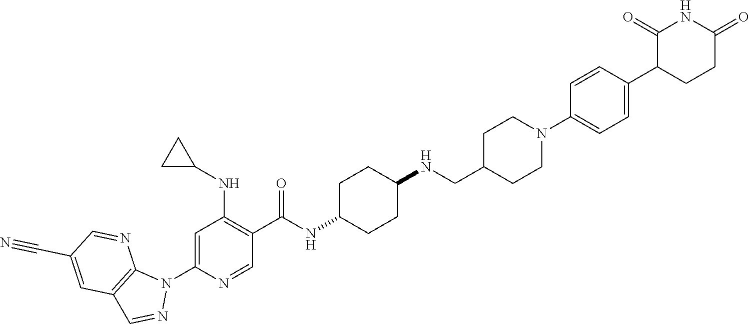 Use tert-butyl N-[4-amino-cyclohexyl]carbamate in step 1 | 34% yield as an off-white solid. LCMS method 2: retention time: 2.083 99.9% purity at 215 nm [M + H]⁺ = 701.4, [M + 2H]²⁺ = 351.3. ¹H NMR (400 MHz, DMSO-d6) δ ppm 0.53-0.59 (m, 2 H), 0.83-0.88 (m, 2 H), 1.12-1.22 (m, 2 H), 1.28-1.42 (m, 4 H), 1.50-1.59 (m, 1 H), 1.73-1.84 (m, 4 H), 1.88- 1.95 (m, 2 H), 1.97-2.04 (m, 1 H), 2.07-2.17 (m, 1 H), 2.21 (s, 3 H), 2.24-2.30 (m, 2 H), 2.32-2.39 (m, 1 H), 2.41-2.48 (m, 1 H), 2.55-2.67 (m, 4 H), 3.63-3.74 (m, 4 H), 6.88 (d, J = 8.6 Hz, 2 H), 7.03 (d, J = 8.6 Hz, 2 H), 7.67 (s, 1 H), 8.38 (d, J = 7.8 Hz, 1 H), 8.54 (s, 1 H), 8.59 (s, 1 H), 8.65 (s, 1 H), 9.00-9.04 (m, 1 H), 9.04-9.08 (m, 1 H), 10.77 (s, 1 H). |
| P-103 | A-10 | C-38 | 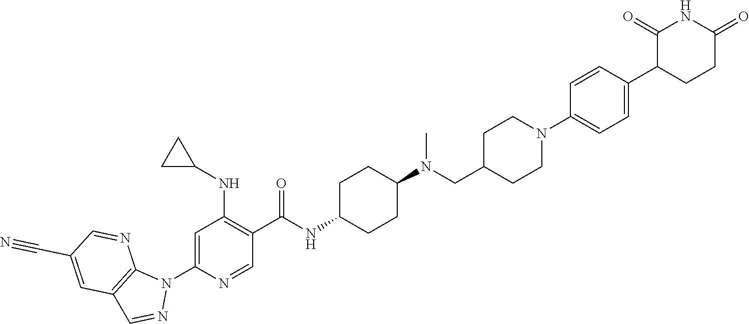 [SMILES: CN(CC1CCN(c2ccc(C3CCC(=O)NC3=O)cc2)CC1)[C@H]1CC[C@H](NC(=O)c2cnc(-n3ncc4cc(C#N)cnc43)cc2NC2CC2)CC1] | 34% yield as an off-white solid. LCMS method 2: Retention time: 1.390 min, 98.5% purity at 215 nm [M + H]⁺ = 715.4. ¹H NMR (400 MHz, DMSO-d6) δ ppm 0.53-0.59 (m, 2 H), 0.83-0.88 (m, 2 H), 1.12-1.22 (m, 2 H), 1.28-1.42 (m, 4 H), 1.50-1.59 (m, 1 H), 1.73-1.84 (m, 4 H), 1.88- 1.95 (m, 2 H), 1.97-2.04 (m, 1 H), 2.07-2.17 (m, 1 H), 2.21 (s, 3 H), 2.24-2.30 (m, 2 H), 2.32-2.39 (m, 1 H), 2.41-2.48 (m, 1 H), 2.55-2.67 (m, 4 H), 3.63-3.74 (m, 4 H), 6.88 (d, J = 8.6 Hz, 2 H), 7.03 (d, J = 8.6 Hz, 2 H), 7.67 (s, 1 H), 8.38 (d, J = 7.8 Hz, 1 H), 8.54 (s, 1 H), 8.59 (s, 1 H), 8.65 (s, 1 H), 9.00-9.04 (m, 1 H), 9.04-9.08 (m, 1 H), 10.77 (s, 1 H). |

General Procedure X-13. The scheme shown below for the synthesis of P-104 is provided as a representative synthesis for General Procedure X-13.

### Example S53. 6-(5-cyanopyrazolo[3,4-b]pyridin-1-yl)-4-(cyclopropylamino)-N-[4-[2-[4-[4-(2,6-dioxo-3-piperidyl)phenyl]piperazin-1-yl]-1,1-difluoro-ethyl]cyclohexyl]pyridine-3-carboxamide (P-104)

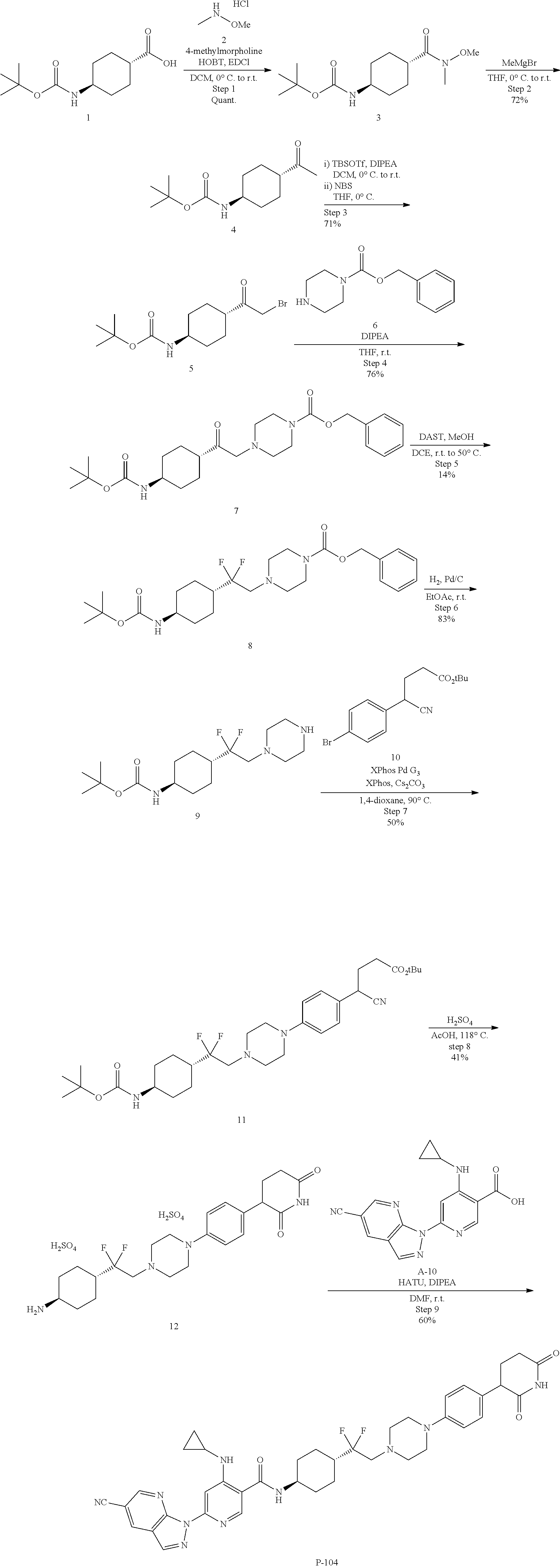

Step 1. Preparation of tert-butyl N-[4-[methoxy(methyl)carbamoyl]cyclohexyl] carbamate (3): To a solution of 4-(tert-butoxycarbonylamino)cyclohexanecarboxylic acid 1 (4.50 g, 18.5 mmol, 1 eq.) in DCM (270 mL) were added N,O-dimethylhydroxylamine hydrochloride 2 (2.9 g, 29.73 mmol, 1.6 eq.) and 4-methylmorpholine (2.45 mL, 22.28 mmol, 1.2 eq.). The reaction was cooled down to 0° C. and EDCI (7.8 g, 40.69 mmol, 2.2 eq.) and HOBT (2.5 g, 18.5 mmol, 1 eq.) were added. After the last addition, the mixture was stirred at room temperature. After 18 h, TLC showed full conversion. The reaction was quenched with 50 mL of an aqueous 10% KHSO₄ solution and 100 mL followed by the addition of MTBE. Layers were separated and the aqueous one was extracted 2 times with MTBE. The combined organics were washed twice with saturated NaHCO₃, twice with brine, dried over sodium sulfate, filtered and concentrated under reduced pressure to give 3 (5.29 g, quantitative yield) as a white solid. ¹H NMR (400 MHz, CDCl₃) δ ppm 1.15 (qd, J=12.6, 3.4 Hz, 2H), 1.43-1.48 (m, 9 H), 1.55-1.69 (m, 2H), 1.83 (br d, J=12.7 Hz, 2H), 2.04-2.14 (m, 2H), 2.55-2.69 (m, 1H), 3.18 (s, 3H), 3.35-3.54 (m, 1H), 3.70 (s, 3H), 4.27-4.49 (m, 1H).

Step 2. Preparation of tert-butyl N-(4-acetylcyclohexyl)carbamate (4): To a round-bottom flask were introduced tert-butyl N-[4-[methoxy(methyl)carbamoyl]cyclohexyl]carbamate 3 (5.29 g, 18.47 mmol, 1 eq.) and THF (180 mL). The reaction was cooled down to 0° C. and MeMgBr (18.47 mL, 55.42 mmol, 3 eq.) was added dropwise. After the addition, the solution was allowed to warm to room temperature. After 2 h, TLC showed full conversion. The reaction was quenched with a solution of saturated ammonium chloride and the resulting mixture was stirred at room temperature. After 15 min, water was added, the layers were separated and the aqueous phase extracted 3 times with ethyl acetate. The combined organics were dried over magnesium sulfate, filtered and evaporated under reduced pressure. The residue was then purified by normal phase flash chromatography (120 g silica column, preabsorbed, elution: 5 to 40% EtOAc/Heptanes over 20 CV). Fractions were combined and concentrated to give 4 (3.21 g, 72% yield) as a white solid.

¹H NMR (400 MHz, CDCl₃) δ ppm 1.12 (qd, J=12.5, 3.4 Hz, 2H), 1.38-1.49 (m, 2H), 1.45 (s, 9H), 1.96 (br d, J=13.2 Hz, 2H), 2.07-2.13 (m, 2H), 2.14 (s, 3H), 2.22-2.33 (m, 1H), 3.39 (br d, J=3.4 Hz, 1H), 4.39 (br d, J=1.7 Hz, 1H).

Step 3. Preparation of tert-butyl N-[4-(2-bromoacetyl)cyclohexyl]carbamate (5): To a solution of tert-butyl N-(4-acetylcyclohexyl)carbamate 4 (3.20 g, 13.26 mmol, 1 eq.) in DCM (64 mL) at 0° C. were successively added dropwise DIPEA (9.24 mL, 53.04 mmol, 4 eq.) and tert-butyldimethylsilyl trifluoromethanesulfonate (7.31 mL, 31.82 mmol, 2.4 eq.). After the last addition the reaction mixture was allowed to warm to room temperature. After 18 h, TLC showed full conversion. The reaction was quenched with water. Layers were separated and the aqueous one extracted 3 times with DCM. The combined organics were washed with brine, dried over sodium sulfate, filtered and evaporated under reduced pressure to give an orange semi-solid. This intermediate was taken up with THF (48.8 mL) and cooled down to 0° C. NBS (4.96 g, 27.85 mmol, 2.1 eq.) was added and the resulting mixture was stirred at 0° C. After 4 h, TLC showed full conversion. The reaction was poured onto a saturated NaHCO₃ solution and diluted with ethyl acetate. Phases were separated and the aqueous one extracted 3 times with ethyl acetate. The combined organics were washed once with brine, dried over sodium sulfate, filtered and evaporated under reduced pressure. The residue was then purified by normal phase flash chromatography (120 g silica column, preabsorbed, elution: 0 to 30% EtOAc/Heptanes over 20 CV). Fractions were combined and concentrated to give 5 (3.0 g, 71% yield) as a light-orange solid.

¹H NMR (400 MHz, CDCl₃) δ ppm 1.16 (qd, J=12.6, 3.2 Hz, 2H), 1.45 (s, 9H), 1.51 (dd, J=12.5, 2.7 Hz, 2H), 1.97 (d, J=13.2 Hz, 2H), 2.08-2.16 (m, 2H), 2.63-2.73 (m, 1H), 3.35-3.48 (m, 1H), 3.95 (s, 2H), 4.33-4.44 (m, 1H).

Step 4. Preparation of benzyl 4-[2-[4-(tert-butoxycarbonylamino)cyclohexyl]-2-oxo-ethyl]piperazine-1-carboxylate (7): A solution of tert-butyl N-[4-(2-bromoacetyl)cyclohexyl]carbamate 5 (2.00 g, 6.25 mmol, 1 eq.), benzyl piperazine-1-carboxylate 6 (1.65 g, 7.49 mmol, 1.2 eq.), DIPEA (3.26 mL, 18.74 mmol, 3 eq.) and THF (25 mL) was stirred at room temperature. After 18 h, TLC showed full conversion. Ethyl acetate and water were added to the reaction and phases were separated. The aqueous one was extracted 3 times with ethyl acetate, the combined organics were washed with water/brine 1/1, dried over sodium sulfate, filtered and evaporated under reduced pressure. The residue was then purified by normal phase flash chromatography (80 g silica column, preabsorbed, elution: 0 to 100% MeOH/DCM over 30 CV). Fractions were combined and concentrated to give 7 (2.30 g, 76% yield) as a light-orange solid.

LCMS method 1: 94.6% purity at 215 nm, [M+H]⁺=460.6.

¹H NMR (400 MHz, CDCl₃) δ ppm 1.12 (qd, J=12.6, 3.2 Hz, 2H), 1.40-1.54 (m, 11H), 1.85-1.89 (m, 2H), 2.05-2.14 (m, 2H), 2.32-2.40 (m, 1H), 2.41-2.49 (m, 4H), 3.27 (s, 2H), 3.34-3.47 (m, 1H), 3.51-3.59 (m, 4H), 4.35-4.40 (m, 1H), 5.13 (s, 2H), 7.30-7.39 (m, 5H).

Step 5. Preparation of benzyl 4-[2-[4-(tert-butoxycarbonylamino)cyclohexyl]-2,2-difluoro-ethyl]piperazine-1-carboxylate (8): To a flame-dried round-bottom flask were introduced DAST (2.6 mL, 19.68 mmol, 3.9 eq.), methanol (25 uL, 0.62 mmol, 0.12 eq.) and DCE (13 mL) and the mixture was stirred at room temperature. After 5 min, benzyl 4-[2-[4-(tert-butoxycarbonylamino)cyclohexyl]-2-oxo-ethyl]piperazine-1-carboxylate 7 (2.30 g, 5.00 mmol, 1 eq.) was added and the reaction was stirred at 50° C. After 8 h, LCMS showed full conversion. Ethyl acetate and water were added to the reaction and phases were separated. The aqueous one was extracted 3 times with ethyl acetate, the combined organics was washed twice with water/brine 1/1, dried over magnesium sulfate, filtered and evaporated under reduced pressure. The residue was purified by reverse phase flash chromatography (100 g C18 column, liquid deposit (DMSO), elution: 5% MeOH/0.1% HCOOH over 3 CV, then 5 to 60% MeOH/0.1% HCOOH over 15 CV). Fractions were combined and concentrated to give 8 (342 mg, 14% yield) as an orange semi-solid.

LCMS method 1: 99.9% purity at 215 nm, [M+H]⁺=482.4.

¹H NMR (400 MHz, CDCl₃) δ ppm 1.04-1.16 (m, 2H), 1.31-1.50 (m, 11H), 1.83-1.92 (m, 3H), 2.04-2.12 (m, 2H), 2.51-2.59 (m, 4H), 2.65-2.76 (m, 2H), 3.33-3.44 (m, 1H), 3.47-3.54 (m, 4H), 4.31-4.43 (m, 1H), 5.14 (s, 2H), 7.32-7.41 (m, 5H).

¹⁹F NMR (377 MHz, CDCl₃) δ ppm −104.72 (br s, 2 F).

Step 6. Preparation of tert-butyl N-[4-(1,1-difluoro-2-piperazin-1-yl-ethyl)cyclohexyl]carbamate (9): To a round-bottom flask were introduced benzyl 4-[2-[4-(tert-butoxycarbonylamino)cyclohexyl]-2,2-difluoro-ethyl]piperazine-1-carboxylate 8 (340 mg, 0.71 mmol, 1 eq.), palladium on carbon (3.0 g, 1.41 mmol, 2 eq.) and ethyl acetate (25 mL). The reaction was vigorously sparged with nitrogen for 15 min followed by hydrogen for 10 min. The reaction was stirred under hydrogen atmosphere (1 atm) at room temperature. After 4.5 h, LCMS showed full conversion. The mixture was filtered over a pad of celite and the cake was washed with ethyl acetate and thoroughly with methanol. The filtrate was evaporated under reduced pressure to give 9 (207 mg, 83% yield) as a light-brown solid.

LCMS method 1: 99.9% purity at 215 nm, [M+H]⁺=348.2.

¹H NMR (400 MHz, CDCl₃) δ ppm 1.04-1.18 (m, 2H), 1.30-1.39 (m, 2H), 1.45 (s, 9H), 1.77-1.90 (m, 3H), 2.05-2.11 (m, 2H), 2.78 (t, J=13.9 Hz, 2H), 2.90-2.97 (m, 4H), 3.20-3.28 (m, 4H), 3.35-3.40 (m, 1H), 4.40 (d, J=7.6 Hz, 1H), 9.67 (br s, 1H).

¹⁹F NMR (377 MHz, CDCl₃) δ ppm −105.71 (br s, 2 F).

Step 7. Preparation of tert-butyl 4-[4-[4-[2-[4-(tert-butoxycarbonylamino)cyclohexyl]-2,2-difluoro-ethyl]piperazin-1-yl]phenyl]-4-cyano-butanoate (11): To a flame-dried sealed tube were introduced tert-butyl N-[4-(1,1-difluoro-2-piperazin-1-yl-ethyl)cyclohexyl]carbamate 9 (145 mg, 0.42 mmol, 1 eq.), tert-butyl 4-(4-bromophenyl)-4-cyano-butanoate 10 (149 mg, 0.46 mmol, 1.1 eq.), XPhos Pd G3 (35 mg, 0.042 mmol, 0.1 eq.), Cs₂CO₃ (272 mg, 0.83 mmol, 2 eq.), XPhos (10 mg, 0.021 mmol, 0.05 eq.) and 1,4-dioxane (3.2 mL). The reaction was sparged with nitrogen for 15 min under sonication and the reaction was then stirred overnight at 90° C. After 24 h, LCMS showed conversion almost complete. Ethyl acetate and water were added to the reaction and phases were separated. The aqueous one was extracted 3 times with ethyl acetate, then the combined organics were washed with brine, dried over magnesium sulfate, filtered and evaporated under reduced pressure. The residue was then purified by normal phase flash chromatography (40 g silica column, preabsorbed, elution: 0 to 20% EtOAc/Heptanes over 20 CV, then 20% EtOAc/Heptanes over 3 CV). Fractions were combined and concentrated to give 11 (128.6 mg, 50% yield) as a tan solid.

LCMS method 1: 95.6% purity at 215 nm, [M−tBu+H]⁺=535.3.

¹H NMR (400 MHz, CDCl₃) δ ppm 1.05-1.17 (m, 2H), 1.38-1.46 (s, 20H), 1.87-1.92 (m, 2H), 2.06-2.18 (m, 4H), 2.33-2.45 (m, 2H), 2.70-2.77 (m, 6H), 3.14-3.23 (m, 4H), 3.35-3.45 (m, 1H), 3.87 (t, J=7.3 Hz, 1H), 4.34-4.40 (m, 1H), 6.90 (d, J=8.8 Hz, 2H), 7.21 (d, J=8.6 Hz, 2H).

¹⁹F NMR (377 MHz, CDCl₃) δ ppm −104.50 (br s, 2 F).

Step 8. Preparation of 3-[4-[4-[2-(4-aminocyclohexyl)-2,2-difluoro-ethyl]piperazin-1-yl]phenyl]piperidine-2,6-dione;sulfuric acid (12): To a solution of tert-butyl 4-[4-[4-[2-[4-(tert-butoxycarbonylamino)cyclohexyl]-2,2-difluoro-ethyl]piperazin-1-yl]phenyl]-4-cyano-butanoate 11 (128 mg, 0.22 mmol, 1 eq.) in acetic acid (1.2 mL) was added concentrated sulfuric acid (35 uL, 0.65 mmol, 3 eq.). The mixture was stirred at 118° C. After 2 h, LCMS showed full conversion. Volatiles were removed under vacuum and the residue was purified by reverse phase flash chromatography (30 g C18 column, liquid deposit (DMSO), elution: 5% MeCN/0.1% HCOOH over 5 CV, then 5 to 40% MeCN/0.1% HCOOH over 20 CV, then 40% MeCN/0.1% HCOOH over 5 CV, then 40 to 100% MeCN/0.1% HCOOH over 10 CV, then 100% MeCN/0.1% HCOOH over 5 CV). Fractions were combined and concentrated to give 12 (55.8 mg, 41% yield) as a white solid as a full bis sulfuric acid salt.

LCMS method 1: 99.9% purity at 215 nm, [M−2H₂SO₄+H]⁺=435.3.

¹H NMR (400 MHz, DMSO-d6) δ ppm 1.22-1.29 (m, 5H), 1.82-2.04 (m, 6H), 2.05-2.19 (m, 1H), 2.58-2.69 (m, 6H), 2.73-2.90 (m, 3H), 3.08-3.13 (m, 4H), 3.73 (dd, J=10.8, 4.6 Hz, 1H), 6.88 (d, J=8.8 Hz, 2H), 7.05 (d, J=8.8 Hz, 2H), 8.32 (s, 2H), 10.77 (br s, 1H).

¹⁹F NMR (377 MHz, DMSO-d6) δ ppm −103.72 (br s, 2 F).

Step 9. Preparation of 6-(5-cyanopyrazolo[3,4-b]pyridin-1-yl)-4-(cyclopropylamino)-N-[4-[2-[4-[4-(2,6-dioxo-3-piperidyl)phenyl]piperazin-1-yl]-1,1-difluoro-ethyl]cyclohexyl]pyridine-3-carboxamide (P-104): To an iced-cold solution of 3-[4-[4-[2-(4-aminocyclohexyl)-2,2-difluoro-ethyl]piperazin-1-yl]phenyl]piperidine-2,6-dione;sulfuric acid 12 (55.6 mg, 0.088 mmol, 1.1 eq.), 6-(5-cyanopyrazolo[3,4-b]pyridin-1-yl)-4-(cyclopropylamino)pyridine-3-carboxylic acid A-10 (25.7 mg, 0.080 mmol, 1 eq.) and DIPEA (140 uL, 0.804 mmol, 10 eq.) in DMF (2.7 mL) was added HATU (36.6 mg, 0.096 mmol, 1.2 eq.). After the addition, the reaction was allowed to stir at room temperature. After 18 h, LCMS showed no conversion. An additional DIPEA (140 uL, 0.804 mmol, 10 eq.) and HATU (36.6 mg, 0.096 mmol, 1.2 eq.) were added and the reaction mixture was stirred at room temperature. After 1 h, LCMS showed full conversion. Ethyl acetate and water were added to the reaction and the phases were separated. The aqueous layer was extracted 3 times with ethyl acetate, then the combined organics were washed with brine, dried over MgSO₄, filtered and evaporated under reduced pressure. The residue was purified by reverse phase flash chromatography (30 g C18 column, liquid deposit (DMSO), elution: 5% MeCN/0.1% HCOOH over 5 CV, then 5 to 100% MeCN/0.1% HCOOH over 25 CV). Fractions were combined, concentrated and lyophilised to give P-104 (35.4 mg, 60% yield) as an off-white solid.

LCMS method 3: 95.5% purity at 215 nm, [M+H]⁺=737.4.

¹H NMR (400 MHz, DMSO-d6) δ ppm 0.52-0.60 (m, 2H), 0.81-0.89 (m, 2H), 1.12-1.44 (m, 5H), 1.85-2.05 (m, 5H), 2.07-2.20 (m, 1H), 2.41-2.47 (m, 1H), 2.53-2.65 (m, 2H), 2.65-2.72 (m, 4H), 2.82 (br t, J=14.9 Hz, 2H), 3.06-3.17 (m, 4H), 3.66-3.80 (m, 2H), 6.89 (d, J=8.6 Hz, 2H), 7.05 (d, J=8.3 Hz, 2H), 7.67 (s, 1H), 8.41 (d, J=7.6 Hz, 1H), 8.54 (s, 1H), 8.59 (s, 1H), 8.65 (s, 1H), 9.00-9.04 (m, 1H), 9.04-9.08 (m, 1H), 10.77 (s, 1H).

¹⁹F NMR (377 MHz, DMSO-d6) δ ppm −103.55 (s, 2 F).

Table 42 summarizes the compounds prepared using General Procedure X-13.

TABLE 42
Final Compounds Prepared via General Procedure X-13

| Com- | | | | |
|---|---|---|---|---|
| pound | TMB | CBM | | |
| No. | Portion | Portion | Structure | Characterization |
| P-104 | A-10 | | 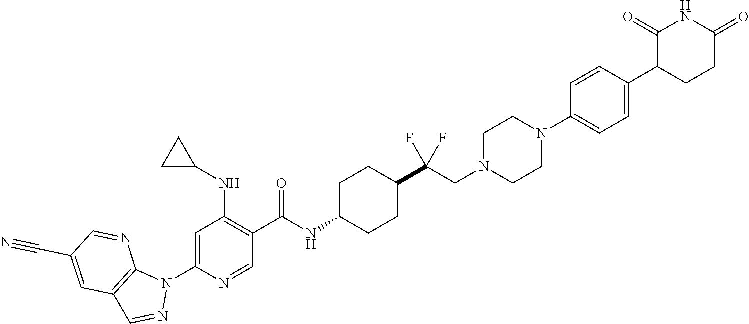 [SMILES: N#Cc1cnc2c(cnn2-c2cc(NC3CC3)c(C(=O)N[C@H]3CC[C@H](C(F)(F)CN4CCN(c5ccc(C6CCC(=O)NC6=O)cc5)CC4)CC3)cn2)c1] | 51% yield as an off-white solid. LCMS method 2: retention time: 2.871, 95.5% purity at 215 nm [M + H]⁺ = 737.4; [M + 2H]²⁺ = 369.3 ¹H NMR (400 MHz, DMSO-d6) δ ppm 0.52-0.60 (m, 2 H), 0.81- 0.89 (m, 2 H), 1.12-1.44 (m, 5 H), 1.85-2.05 (m, 5 H), 2.07-2.20 (m, 1 H), 2.41-2.47 (m, 1 H), 2.53-2.65 (m, 2 H), 2.65-2.72 (m, 4 H), 2.82 (br t, J = 14.9 Hz, 2 H), 3.06-3.17 (m, 4 H), 3.66-3.80 (m, 2 H), 6.89 (d, J = 8.6 Hz, 2 H), 7.05 (d, J = 8.3 Hz, 2 H), 7.67 (s, 1 H), 8.41 (d, J = 7.6 Hz, 1 H), 8.54 (s, 1 H), 8.59 (s, 1 H), 8.65 (s, 1 H), 9.00-9.04 (m, 1 H), 9.04-9.08 (m, 1 H), 10.77 (s, 1 H). ¹⁹F NMR (377 MHz, DMSO-d6) δ ppm −103.55 (s, 2 F). |

General Procedure X-14. The scheme shown below for the synthesis of P-110 is provided as a representative synthesis for General Procedure X-14.

### Example S54. 6-(5-cyanopyrazolo[3,4-b]pyridin-1-yl)-4-(cyclopropylamino)-N-[4-[2-[4-[4-(2,6-dioxo-3-piperidyl)phenyl]piperazin-1-yl]-1,1-difluoro-ethyl]cyclohexyl]pyridine-3-carboxamide (P-110/111)

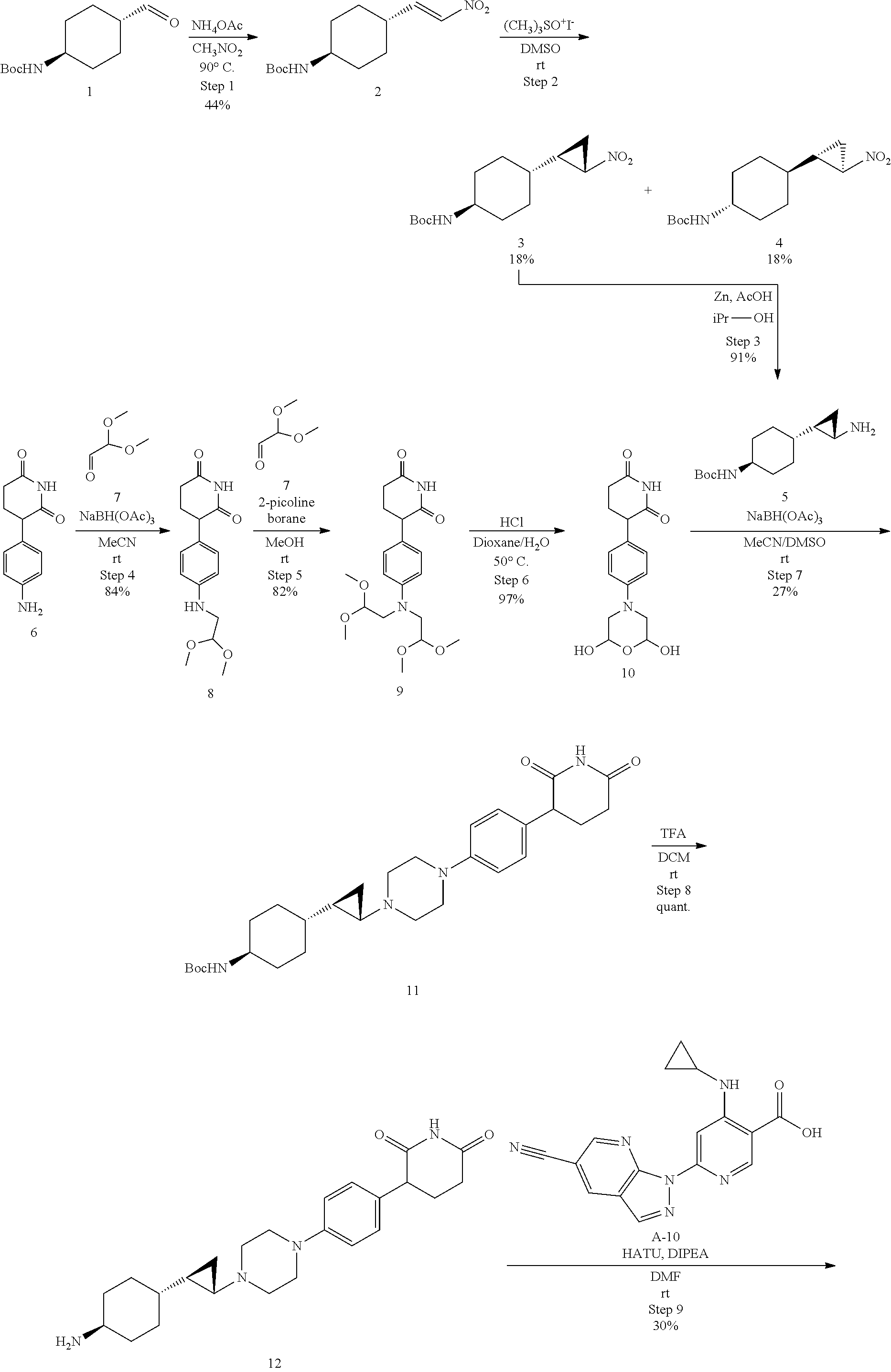

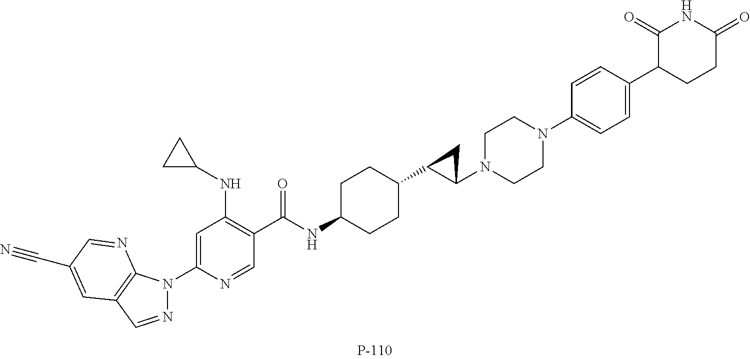 [SMILES: N#Cc1cnc2c(cnn2-c2cc(NC3CC3)c(C(=O)N[C@H]3CC[C@H]([C@H]4C[C@@H]4N4CCN(c5ccc(C6CCC(=O)NC6=O)cc5)CC4)CC3)cn2)c1]

Step 1. Preparation of tert-butyl N-[4-[(E)-2-nitrovinyl]cyclohexyl]carbamate (2): Tert-Butyl N-(4-formylcyclohexyl)carbamate 1 (450 mg, 1.98 mmol), was added to a stirred solution of NH₄OAc (38 mg, 0.49 mmol) in nitromethane (3.5 mL). The mixture was heated at 90° C. The reaction was followed by TLC. After 4.5 h the mixture was cooled and then poured into water and extracted to MTBE (3×10 mL). The combined extracts were washed with brine, dried over Na₂SO₄, filtered and evaporated under reduced pressure. The residue was purified on silica gel (0-40% EtOAc/heptanes) to afford tert-butyl N-[4-[(E)-2-nitrovinyl]cyclohexyl]carbamate 2 (240 mg, 44% yield) as a white solid.

LCMS method 1: 99.9% purity at 215 nm, [M+Na]⁺=293.2.

¹H NMR (400 MHz, CDCl₃) δ ppm 1.12-1.25 (m, 2H), 1.26-1.40 (m, 3H), 1.45 (s, 9H), 1.66-1.75 (m, 1H), 2.17-2.30 (m, 1H), 3.36-3.51 (m, 1H), 4.30-4.49 (m, 1H), 6.95 (dd, J=13.4, 1.2 Hz, 1H), 7.20 (dd, J=13.4, 7.3 Hz, 1H).

Step 2. Preparation of tert-butyl ((1r,4r)-4-((1R,2S)-2-nitrocyclopropyl)cyclohexyl)carbamate (3) and tert-butyl N-[4-[(1R,2S)-2-nitrocyclopropyl]cyclohexyl]carbamate (3): NaH (58 mg, 2.44 mmol) was added to a solution of trimethylsulfoxonium iodide (537 mg, 2.44 mmol) in anhydrous DMSO (15 mL) and the mixture was stirred for 3 h at rt. Then reaction was cooled to 0° C. and a solution of tert-butyl N-[4-[(E)-2-nitrovinyl]cyclohexyl]carbamate 2 (600 mg, 2.22 mmol) in anhydrous DMSO (7 mL) was added slowly before warming up to RT and stirring for 3 h. The reaction mixture was poured into water and extracted to MTBE (3×50 mL). The combined extracts were washed with brine, dried over Na₂SO₄, filtered and evaporated under reduced pressure. The residue was purified on silica gel (EtOAc/heptanes). Desired products were eluted in 35% EtOAc as a mixture of two enantiomers. The enantiomers were separated by chiral SFC (250×21.5 mm Phenomenex Lux® 5 μm Amylose-1 column, 1-10% MeOH as cosolvent). First eluting compound arbitrarily assigned as tert-butyl ((1r,4r)-4-((1R,2S)-2-nitrocyclopropyl)cyclohexyl)carbamate (3) (114 mg, 18% yield) and second eluting compound as tert-butyl ((1S,4r)-4-((1S,2R)-2-nitrocyclopropyl)cyclohexyl)carbamate (4) (109 mg, 18% yield), respectively.

LCMS method 3: 99.9% purity at 215 nm, [M+H]⁺=185.2.

¹H NMR (400 MHz, CDCl₃) δ ppm 0.70-0.86 (m, 1H), 0.95-1.13 (m, 2H), 1.17-1.33 (m, 2H), 1.45 (s, 9H), 1.72-1.92 (m, 4H), 2.00-2.10 (m, 2H), 3.39 (br. s, 1H), 4.02-4.13 (m, 1H), 4.36 (br. s, 1H).

LCMS method 3: 99.9% purity at 215 nm, [M+H]⁺=185.2.

¹H NMR (400 MHz, CDCl₃) δ ppm 0.73-0.88 (m, 1H), 0.98-1.15 (m, 2H), 1.18-1.32 (m, 2H), 1.45 (s, 9H), 1.74-1.92 (m, 4H), 1.99-2.11 (m, 2H), 3.39 (br. s, 1H), 4.05-4.11 (m, 1H), 4.35 (br. s, 1H).

Step 3. Preparation of tert-butyl ((1r,4r)-4-((1R,2S)-2-aminocyclopropyl)cyclohexyl)carbamate (5): Acetic acid (0.44 mL, 7.74 mmol) was added to a suspension of tert-butyl ((1r,4r)-4-((1R,2S)-2-nitrocyclopropyl)cyclohexyl)carbamate 4 (220 mg, 0.77 mmol) and activated Zinc (1.01 g, 15.47 mmol) in iPr-OH (4.0 mL). (Zinc was activated as following: Zinc powder was treated with 0.1 N HCl, washed with water, acetone and MeOH and dried under vacuum for 2 h. The activated zinc was used immediately after preparation.) The mixture was stirred for 3 h. TLC (30% MeOH in EtOAc) showed complete conversion. The reaction mixture was treated with saturated sodium hydroxide up to pH 8. The precipitate was filtered off, washed with MeTHF. The phases of filtrate were separated and the aqueous phase was extracted to MeTHF 2×. The combined organics were washed with brine, dried over magnesium sulfate, and the volatiles evaporated to afford tert-butyl ((1r,4r)-4-((1R,2S)-2-aminocyclopropyl)cyclohexyl)carbamate 5 (180 mg, 91% yield) as a white solid.

¹H NMR (400 MHz, DMSO-d6) δ ppm 0.11-0.26 (m, 2H), 0.27-0.49 (m, 2H),

1.12 (m, 5H), 1.30-1.41 (m, 9H), 1.52-2.00 (m, 5H), 3.06-3.20 (m, 1H), 6.57-6.74 (m, 1H), 6.87 (s, 1H).

Step 4. Preparation of 3-[4-(2,2-dimethoxyethylamino)phenyl]piperidine-2,6-dione (8): A solution of 3-[4-(2,2-dimethoxyethylamino)phenyl]piperidine-2,6-dione 6 (120 mg, 0.41 mmol) and 60% 2,2-dimethoxyacetaldehyde 7 in H₂O (0.12 mL, 0.82 mmol) in MeCN (4.0 mL) was stirred at RT for 10 min. Then NaBH(OAc)₃ (2.07 g, 9.79 mmol) was added and the mixture stirred at RT for 2 h. The reaction was quenched by adding water and most of MeCN evaporated in reduced pressure. The residue was extracted to DCM 3×. Combined extracts were dried over sodium sulfate and concentrated in vacuum to afford 3-[4-(2,2-dimethoxyethylamino)phenyl]piperidine-2,6-dione 8 (1.31 g, 84% yield) as a colorless semi-solid.

LCMS method 1: 91% purity at 215 nm, [M+H]⁺=293.4.

Step 5. Preparation of 3-[4-[bis(2,2-dimethoxyethyl)amino]phenyl]piperidine-2,6-dione (9): A solution of 3-[4-(2,2-dimethoxyethylamino)phenyl]piperidine-2,6-dione 8 (120 mg, 0.41 mmol) and 60% 2,2-dimethoxyacetaldehyde in H₂O (0.12 mL, 0.82 mmol) in a mixture of AcOH (0.4 mL) and MeOH (4.0 mL) was stirred at RT for 10 min. Then 2-Picoline borane complex (42.67 mg, 0.4100 mmol) was added and the mixture stirred at rt. After 16 h stirring, reaction quenched by 5% citric acid and most of MeCN evaporated in vacuum. The mixture purified with reversed phase C18 column chromatography (MeOH/0.1% aqueous formic acid) to afford 3-[4-[bis(2,2-dimethoxyethyl)amino]phenyl]piperidine-2,6-dione 9 (1.46 g, 82% yield) as a colorless semi-solid.

LCMS method 1: 96% purity at 215 nm, [M+H]⁺=381.4.

¹H NMR (400 MHz, DMSO-d6) δ ppm 1.94-2.04 (m, 1H), 2.05-2.18 (m, 1H), 2.41-2.48 (m, 1H), 2.55-2.70 (m, 1H), 3.27-3.37 (m, 12H), 3.43 (d, J=4.9 Hz, 4H), 3.68 (dd, J=11.0, 4.9 Hz, 1H), 4.48 (s, 2H), 6.68 (d, J=8.6 Hz, 2H), 7.00 (d, J=8.6 Hz, 2H), (s, 1H).

Step 6. Preparation of 3-[4-(2,6-dihydroxymorpholin-4-yl)phenyl]piperidine-2,6-dione (10): To a solution of 3-[4-[bis(2,2-dimethoxyethyl)amino]phenyl]piperidine-2,6-dione 9 (650 mg, 1.71 mmol) in 4 M HCl in dioxane (9.75 mL, 39 mmol) was added water (1 mL) and the mixture was heated for 1 h at 50° C. Upon completion of reaction by LCMS, the volatiles evaporated under reduced pressure and the residue was co-evaporated with MeCN 3× to afford 3-[4-(2,6-dihydroxymorpholin-4-yl)phenyl]piperidine-2,6-dione 10 (521 mg, 97% yield) as a brown solid.

LCMS method 1: 97.8% purity at 215 nm, [M+H]⁺=307.4.

Step 7. Preparation of tert-butyl ((1r,4r)-4-((1R,2S)-2-(4-(4-(2,6-dioxopiperidin-3-yl)phenyl)piperazin-1-yl)cyclopropyl)cyclohexyl)carbamate (11): A solution of 3-[4-(2,6-dihydroxymorpholin-4-yl)phenyl]piperidine-2,6-dione 10 (182 mg, 0.59 mmol) and tert-butyl ((1r,4r)-4-((1R,2S)-2-aminocyclopropyl)cyclohexyl)carbamate (180 mg, 0.42 mmol) in a mixture of DMSO (1 mL) and MeCN (3 mL) was stirred at room temp for 10 min. Then NaBH(OAc)₃ (269 mg, 1.27 mmol) was added and the mixture stirred at rt. After 16 h, volatiles were evaporated under reduced pressure and the residue was purified by reversed phase C18 column chromatography (MeCN/0.1% aqueous formic acid) to afford tert-butyl ((1r,4r)-4-((1R,2S)-2-(4-(4-(2,6-dioxopiperidin-3-yl)phenyl)piperazin-1-yl)cyclopropyl)cyclohexyl)carbamate 11 (60 mg, 27% yield) as an off-white solid.

LCMS method 1: 99.9% purity at 215 nm, [M+H]⁺=511.3.

¹H NMR (400 MHz, DMSO-d6) δ ppm 0.27-0.34 (m, 1H), 0.40-0.56 (m, 3H), 1.16 (m, 4H), 1.37 (s, 9H), 1.66-1.82 (m, 4H), 1.95-2.04 (m, 1H), 2.05-2.18 (m, 1H), 2.40-2.48 (m, 1H), 2.54 (s, 1H), 2.61 (m, 4H), 2.64-2.70 (m, 1H), 2.95-3.10 (m, 4H), 3.11-3.23 (m, 1H), 3.72 (dd, J=10.9, 4.8 Hz, 1H), 6.66 (d, J=7.6 Hz, 1H), 6.87 (d, J=8.6 Hz, 2H), 7.04 (d, J=8.6 Hz, 2H), 10.76 (s, 1H).

Step 8. Preparation of 3-(4-(4-((1S,2R)-2-((1r,4r)-4-aminocyclohexyl)cyclopropyl)piperazin-1-yl)phenyl)piperidine-2,6-dione (12): To a solution of tert-butyl ((1r,4r)-4-((1R,2S)-2-(4-(4-(2,6-dioxopiperidin-3-yl)phenyl)piperazin-1-yl)cyclopropyl)cyclohexyl)carbamate 11 (60 mg, 0.12 mmol) in DCM (1.0 mL) was added TFA (1.8 mL, 23.5 mmol) and the mixture was stirred at RT for 20 min. The volatiles were evaporated under reduced pressure and the residue was co-evaporated with MeCN (3×) to afford tert-butyl ((1r,4r)-4-((1S,2R)-2-(4-(4-(2,6-dioxopiperidin-3-yl)phenyl)piperazin-1-yl)cyclopropyl)cyclohexyl)carbamate 12 (75 mg, quantitative) as an off-white solid.

LCMS method 1: 99.9% purity at 215 nm, [M+2H]²⁺=206.2.

Step 9. Preparation of 6-(5-cyanopyrazolo[3,4-b]pyridin-1-yl)-4-(cyclopropylamino)-N-[4-[(1R,2S)-2-[4-[4-(2,6-dioxo-3-piperidyl)phenyl]piperazin-1-yl]cyclopropyl]cyclohexyl]pyridine-3-carboxamide (P-110): To a solution of 3-(4-(4-((1 S,2R)-2-((1r,4r)-4-aminocyclohexyl)cyclopropyl)piperazin-1-yl)phenyl)piperidine-2,6-dione 12 (74 mg, 0.12 mmol), HATU (63 mg, 0.17 mmol) and 6-(5-cyanopyrazolo[3,4-b]pyridin-1-yl)-4-(cyclopropylamino)pyridine-3-carboxylic acid A-10 (40 mg, 0.12 mmol) in DMF (1.25 mL) was added DIPEA (0.27 mL, 1.55 mmol) and reaction mixture stirred for 2 h at rt. The reaction mixture was directly purified by reversed phase C18 column chromatography (MeCN/0.1% aqueous formic acid) to afford 6-(5-cyanopyrazolo[3,4-b]pyridin-1-yl)-4-(cyclopropylamino)-N-[4-[(1R,2S)-2-[4-[4-(2,6-dioxo-3-piperidyl)phenyl]piperazin-1-yl]cyclopropyl]cyclohexyl]pyridine-3-carboxamide P-110 (25.8 mg, 30% yield).

LCMS method 3: 96.1% purity at 215 nm, [M+H]⁺=713.4, [M+2H]²⁺=357.2.

¹H NMR (400 MHz, DMSO-d6) δ ppm 0.32-0.39 (m, 1H), 0.45-0.52 (m, 1H), 0.62 (m, 4H), 0.82-0.89 (m, 2H), 1.07-1.36 (m, 5H), 1.40-1.47 (m, 1H), 1.74-1.82 (m, 1H), 1.82-1.93 (m, 3H), 1.94-2.05 (m, 1H), 2.07-2.19 (m, 1H), 2.53-2.61 (m, 2H), 2.61-2.69 (m, 4H), 3.02-3.12 (m, 4H), 3.72 (dd, J=10.6, 5.0 Hz, 2H), 6.88 (d, J=8.6 Hz, 2H), 7.04 (d, J=8.8 Hz, 2H), 7.67 (s, 1H), 8.38 (d, J=7.6 Hz, 1H), 8.55 (s, 1H), 8.59 (s, 1H), 8.65 (s, 1H), 9.02 (d, J=2.0 Hz, 1H), 9.06 (d, J=2.0 Hz, 1H), 10.77 (s, 1H).

Table 43 summarizes the compounds prepared using General Procedure X-14.

TABLE 43
Final Compounds Prepared via General Procedure X-14

| Com- | | | | |
|---|---|---|---|---|
| pound | TMB | CBM | | |
| No. | Portion | Portion | Structure | Characterization |
| P-110 | A-10 | | 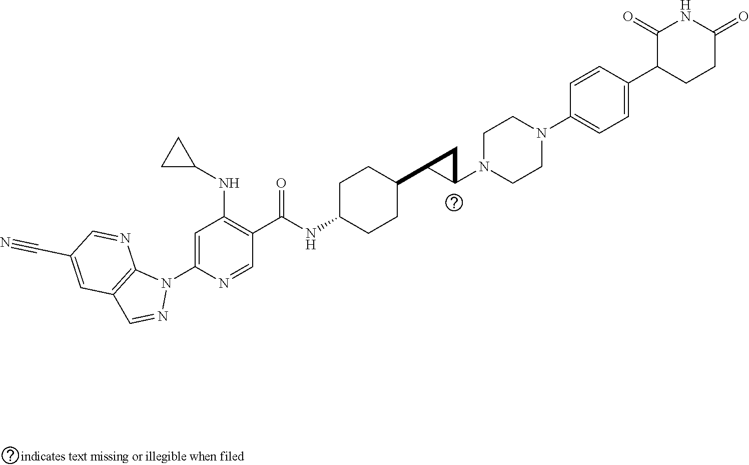 | 30% yield as a white solid. LCMS method 6: Retention time: 3.185 min, 96.1% purity at 215 nm, [M + H]⁺ = 713.4, [M + 2H]²⁺ = 357.2 ¹H NMR (400 MHz, DMSO-d6) δ ppm 0.32-0.39 (m, 1 H), 0.45- 0.52 (m, 1 H), 0.52-0.62 (m, 4 H), 0.82-0.89 (m, 2 H), 1.07- 1.36 (m, 5 H), 1.40-1.47 (m, 1 H), 1.74-1.82 (m, 1 H), 1.82- 1.93 (m, 3 H), 1.94-2.05 (m, 1 H), 2.07-2.19 (m, 1 H), 2.53- 2.61 (m, 2 H), 2.61-2.69 (m, 4 H), 3.02-3.12 (m, 4 H), 3.72 (dd, J = 10.6, 5.0 Hz, 2 H), 6.88 (d, J = 8.6 Hz, 2 H), 7.04 (d, J = 8.8 Hz, 2 H), 7.67 (s, 1 H), 8.38 (d, J = 7.6 Hz, 1 H), 8.55 (s, 1 H), 8.59 (s, 1 H), 8.65 (s, 1 H), 9.02 (d, J = 2.0 Hz, 1 H), 9.06 (d, J = 2.0 Hz, 1 H), 10.77 (s, 1 H). |
| P-111 | A-10 | | 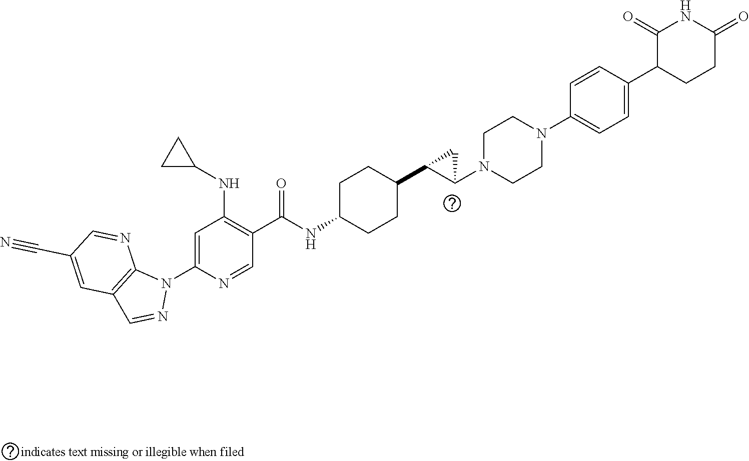 [SMILES: N#Cc1cnc2c(cnn2-c2cc(NC3CC3)c(C(=O)N[C@H]3CC[C@H]([C@@H]4C[C@H]4N4CCN(c5ccc(C6CCC(=O)NC6=O)cc5)CC4)CC3)cn2)c1] | 43% yield as a white solid. LCMS method 2: Retention time: 2.349 min, 94.3% purity at 215 nm, [M + H]⁺ = 713.4, [M + 2H]²⁺ = 357.3 ¹H NMR (400 MHz, DMSO-d6) δ ppm 0.32-0.39 (m, 1 H), 0.46- 0.53 (m, 1 H), 0.53-0.63 (m, 4 H), 0.81-0.89 (m, 2 H), 1.06- 1.37 (m, 5 H), 1.40-1.47 (m, 1 H), 1.74-1.82 (m, 1 H), 1.83- 1.94 (m, 3 H), 1.95-2.05 (m, 1 H), 2.06-2.19 (m, 1 H), 2.53- 2.61 (m, 2 H), 2.61-2.70 (m, 4 H), 3.01-3.13 (m, 4 H), 3.68- 3.79 (m, 2 H), 6.88 (d, J = 8.8 Hz, 2 H), 7.04 (d, J = 8.6 Hz, 2 H), 7.67 (s, 1 H), 8.38 (d, J = 7.6 Hz, 1 H), 8.55 (s, 1 H), 8.59 (s, 1 H), 8.65 (s, 1 H), 9.02 (d, J = 2.0 Hz, 1 H), 9.06 (d, J = 2.0 Hz, 1 H), 10.77 (s, 1 H). |

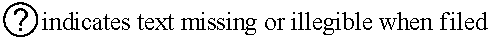

### General Procedure X-15

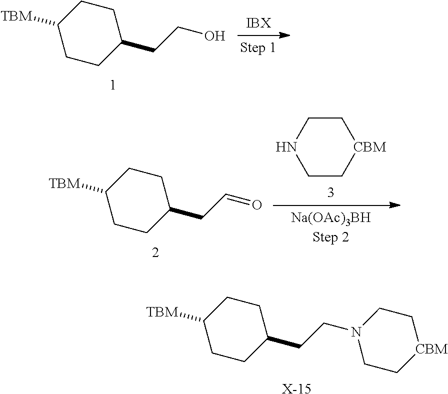

Step 1. Preparation of TBM-aldehyde 2 by oxidation: To a stirred solution of TBM-alcohol 1 (1.0 eq) in DMSO (0.2 M), IBX (3.0 eq.) was added at RT and stirred. The reaction mixture was quenched with sodium bicarbonate solution and extracted with ethyl acetate. The combined organic layer was washed with brine, dried over sodium sulphate, and concentrated under reduced pressure to give aldehyde 2 that was used in the next step without further purification.

Step 2. Preparation X-15 via reductive amination: To a solution of TBM-aldehyde 2 (40 mg, 0.081 mmol) and CBM-amine 3 (1.3 eq.) in DMSO (0.4 M) was added sodium triacetoxyborohydride (1.5 eq.) under nitrogen atmosphere at room temperature. The resulting mixture was stirred at room temperature. The reaction mixture was treated with ice cold water and extracted with DCM. Organic phases were combined and washed with brine. Combined organic phases were dried over anhydrous Na₂SO₄, filtered and the filtrate was concentrated under reduced pressure to give a crude residue. The crude was purified by preparative HPLC to afford product X-15.

### Example S55. 6-amino-1-(4-(((R)-1-cyanoethyl)amino)-5-(4-((1r,4R)-4-(2-(4-(4-(2,6-dioxopiperidin-3-yl)phenyl)piperazin-1-yl)ethyl)cyclohexyl)-1H-1,2,3-triazol-1-yl)pyridin-2-yl)-1H-pyrazolo[3,4-b]pyridine-5-carbonitrile, TFA (P-160)

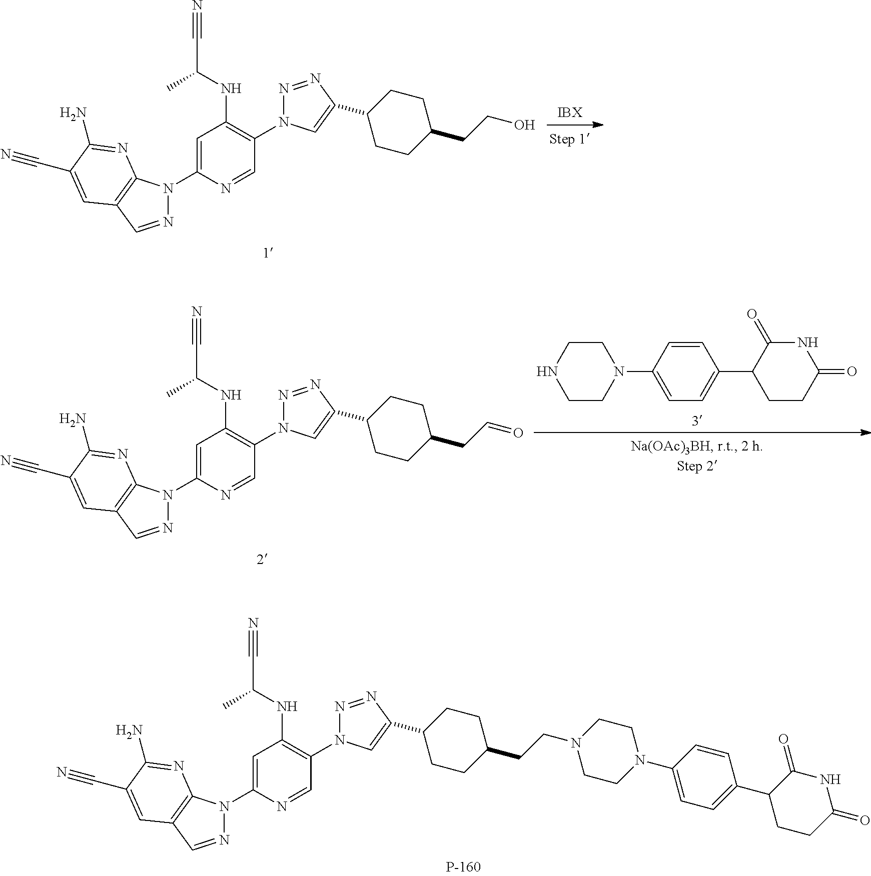

LCMS Method 1. Kinetex XB-C18, 75×3.0 mm, 2.6 μm, Temperature: RT, Flow: 1.0 mL/min, Run Time: 5 minutes, Mobile Phase Conditions: Mobile Phase-A: 5.0 mm Ammonium formate pH 3.3:CH₃CN (98:02), Mobile Phase-B: CH₃CN:5.0 mm Ammonium formate pH 3.3 (98:02), Gradient: Initial 80% Mobile Phase A and 20% Mobile Phase B linear gradient to 100% Mobile Phase B for 4.0 min. MSD positive.

Step 1′. Preparation of 6-amino-1-(4-(((R)-1-cyanoethyl)amino)-5-(4-((1r,4R)-4-(2-oxoethyl)cyclohexyl)-1H-1,2,3-triazol-1-yl)pyridin-2-yl)-1H-pyrazolo[3,4-b]pyridine-5-carbonitrile 2′: To a stirred solution of 6-amino-1-(4-(((R)-1-cyanoethyl)amino)-5-(4-((1r,4R)-4-(2-hydroxyethyl)cyclohexyl)-1H-1,2,3-triazol-1-yl)pyridin-2-yl)-1H-pyrazolo[3,4-b]pyridine-1′ (450 mg, 0.904 mmol) in DMSO (5 mL), IBX (760 mg, 2.71 mmol) was added at RT and stirred for 2 h. The reaction mixture was quenched with sodium bicarbonate solution and extracted with ethyl acetate (3×20 mL). The combined organic layer was washed with brine, dried over sodium sulphate, and concentrated under reduced pressure to give compound 6-amino-1-(4-(((R)-1-cyanoethyl)amino)-5-(4-((1r,4R)-4-(2-oxoethyl)cyclohexyl)-1H-1,2,3-triazol-1-yl)pyridin-2-yl)-1H-pyrazolo[3,4-b]pyridine-5-carbonitrile 2′ (400 mg), which was used in the next step without further purification.

LCMS method 1: retention time: 2.01 min, 89.4% purity at 220 nm, [M+H]⁺=496.2

Step 2′. Preparation of 6-amino-1-(4-(((R)-1-cyanoethyl)amino)-5-(4-((1r,4R)-4-(2-(4-(4-(2,6-dioxopiperidin-3-yl)phenyl)piperazin-1-yl)ethyl)cyclohexyl)-1H-1,2,3-triazol-1-yl)pyridin-2-yl)-1H-pyrazolo[3,4-b]pyridine-5-carbonitrile, TFA. P-160: To a solution of 6-amino-1-(4-(((R)-1-cyanoethyl)amino)-5-(4-((1r,4R)-4-(2-oxoethyl)cyclohexyl)-1H-1,2,3-triazol-1-yl)pyridin-2-yl)-1H-pyrazolo[3,4-b]pyridine-5-carbonitrile 2′ (40 mg, 0.081 mmol) and 3-(4-(piperazin-1-yl)phenyl)piperidine-2,6-dione 3′ (28.7 mg, 0.105 mmol) in DMSO (2.0 mL) was added sodium triacetoxyborohydride (25.7 mg, 0.121 mmol) under nitrogen atmosphere at room temperature. The resulting mixture was stirred at room temperature for 2 h. The reaction mixture was treated with ice cold water (50 mL) and extracted with DCM (2×50 mL). Organic phases were combined and washed with brine (50 mL). Combined organic phases were dried over anhydrous Na₂SO₄, filtered and the filtrate was concentrated under reduced pressure to give a crude residue. The crude was purified by preparative HPLC to afford 22 mg of 6-amino-1-(4-(((R)-1-cyanoethyl)amino)-5-(4-((1r,4R)-4-(2-(4-(4-(2,6-dioxopiperidin-3-yl)phenyl)piperazin-1-yl)ethyl)cyclohexyl)-1H-1,2,3-triazol-1-yl)pyridin-2-yl)-1H-pyrazolo[3, 4-b]pyridine-5-carbonitrile, TFA P-160 (22 mg, 0.025 mmol, 30.9% yield) as an off-white solid. Prep-HPLC method: X-Select C18, 150×19 mm, 5 Mobile phase: A: 0.1% TFA in MQ-water; B: Acetonitrile; Flow rate: 15 mL/min.

LCMS method 1: retention time: 1.267 min, 98.43% purity at 220 nm, [M+H]⁺=753.2.

¹H NMR (400 MHz, DMSO-d6, 80° C.): δ ppm 1.09-1.18 (m, 5H), 1.38-1.45 (m, 3H), 1.66-1.70 (m, 2H), 1.71 (d, J=6.8 Hz, 3H), 1.84-1.87 (m, 2H), 1.92-1.97 (m, 3H), 2.10-2.19 (m, 1H), 2.61-2.68 (m, 2H), 3.45-3.60 (m, 3H), 3.77 (s, 3H), 3.80-3.84 (m, 2H), 4.93-4.96 (m, 1H), 6.66 (brs, 2H), 6.95-7.05 (m, 3H), 7.83 (s, 1H), 8.22 (s, 1H), 8.32 (d, J=7.6 Hz, 1H), 8.52 (s, 1H), 8.68 (s, 1H), 8.74 (d, J=6.8 Hz, 1H), 10.41 (s, 1H).

Table 44 summarizes the compounds prepared using General Procedure X-15.

TABLE 44
Final Compounds Prepared via General Procedure X-15

| Compound | TMB | CBM | | |
|---|---|---|---|---|
| No. | Portion | Portion | Structure | Characterization |
| P-157 | A64 | C12 | 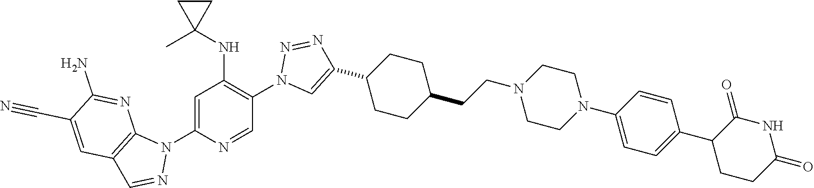 | 44% yield as an off-white solid. LCMS method 7: Retention time: 1.827 min, 93.2% purity at 215 nm, [M + H]⁺ = 754.3, [M + 2H]⁺ = 377.7 ¹H NMR (400 MHz, DMSO-d6, 80 C): δ ppm 0.81-0.84 (m, 4H), 1.19-1.25 (m, 2H), 1.44 (s, 3H), 1.43-1.55 (m, 3H), 1.68-1.70 (m, 2H), 1.90-1.92 (m, 2H), 2.08-2.19 (m, 4H), 2.62-2.67 (m, 2H), 2.73-2.82 (m, 1H), 3.10- 3.13 (m, 3H), 3.24-3.28 (m, 3H), 3.76- 3.80 (m, 2H), 6.79 (brs, 2H), 6.91 (s, 1H), 6.98 (d, J = 8.8 Hz, 2H), 7.15 (d, J = 8.4 Hz, 2H), 7.75 (s, 1H), 8.21 (s, 1H), 8.23 (s, 1H), 8.26 (s, 1H), 8.53 (s, 1H), 10.51 (s, 1H). Four protons were not apparent by ¹H NMR. |
| P-160 | A66 | C12 | 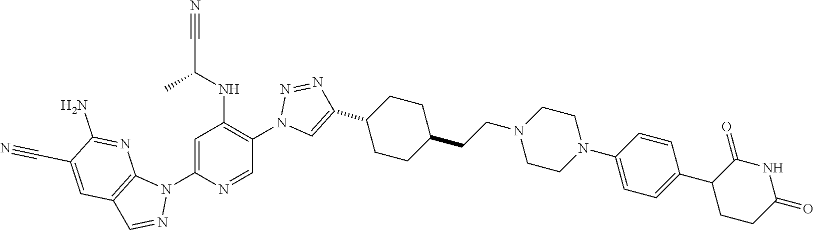 | 31% yield as an off-white solid. LCMS method 7: Retention time: 1.267 min, 98.4% purity at 215 nm, [M + H]⁺ = 753.2 ¹H-NMR (400 MHz, DMSO-d6): δ ppm 1.17- 1.24 (m, 2H), 1.40-1.52 (m, 3H), 1.63 (d, J = 6.8 Hz, 3H), 1.62-1.66 (m, 2H), 1.87- 1.90 (m, 2H), 1.98-2.03 (m, 1H), 2.14-2.18 (m, 3H), 2.75-2.86 (m, 1H), 2.96-2.99 (m, 2H), 3.15-3.20 (m, 2H), 3.25-3.30 (m, 2H), 3.61-3.64 (m, 2H), 3.75-3.80 (m, 1H), 3.83-3.86 (m, 2H), 4.98-5.03 (m, 1H), 6.99 (d, J = 8.8 Hz, 2H), 7.09 (d, J = 8.0 Hz, 1H), 7.13 (d, J = 8.4 Hz, 2H), 7.38 (brs, 2H), 8.00 (s, 1H), 8.25 (s, 1H), 8.32 (s, 1H), 8.35 (s, 1H), 8.59 (s, 1H), 10.81 (s, 1H). |
| P-162 | A67 | C12 | 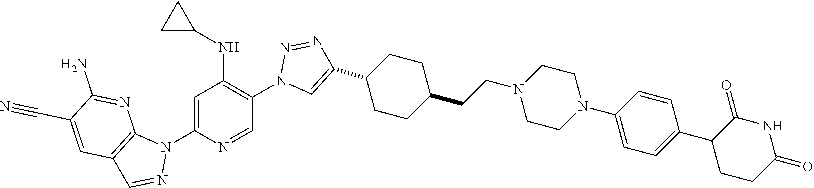 | 19% yield as an off-white solid. LCMS method 7: Retention time: 1.677 min, 99.4% purity at 215 nm, [M + H]⁺ = 740.3, [M + 2H]⁺ = 370.8 ¹H NMR (400 MHz, DMSO-d6): δ ppm 0.56- 0.57 (m, 2H), 0.83-0.85 (m, 2H), 1.16- 1.19 (m, 2H), 1.47-1.51 (m, 3H), 1.64-1.66 (m, 2H), 1.86-2.02 (m, 3H), 2.12-2.16 (m, 3H), 2.66-2.74 (m, 4H), 2.96-2.99 (m, 2H), 3.14-3.26 (m, 4H), 3.60 -3.64 (m, 2H), 3.75- 3.86 (m, 3H), 6.99 (d, J = 8.8 Hz, 2H), 7.10- 7.14 (m, 3H), 7.30 (brs, 2H), 7.96 (s, 2H), 8.23 (s, 1H), 8.25 (s, 1H), 8.30 (s, 1H), 8.58 (s, 1H), 8.58 (s, 1H), 10.81 (s, 1H). Two protons were not apparent by ¹H NMR. |
| P-196 | A-78 | C-12 | 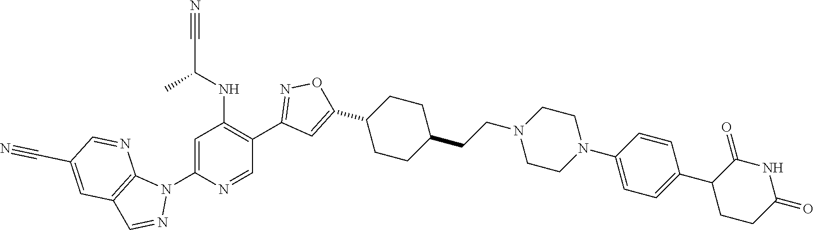 | 11% yield as an off-white solid. LCMS method 7: Retention time: 1.744 min, 94.0% purity at 215 nm, [M + H]⁺ = 738.2 ¹H NMR (400 MHz, DMSO-d6 + TFA): δ ppm 1.10-1.14 (m, 2H), 1.32-1.49 (m, 3H), 1.55-1.60 (m, 2H), 1.75-1.90 (m, 5H), 2.04- 2.10 (m, 4H), 2.78-2.81 (m, 1H), 2.92- 3.20 (m, 6H), 3.52-3.59 (m, 2H), 3.71- 3.76 (m, 2H), 5.10-5.18 (m, 1H), 6.89 (s, 1H), 6.95 (d, J = 8.8 Hz, 2H), 7.10 (d, J = 8.8 Hz, 2H), 8.20 (s, 1H), 8.69-8.73 (m, 2H), 8.91 (d, J = 2.0 Hz, 1H), 9.04 (d, J = 1.6 Hz, 1H). Four protons were not apparent by ¹H NMR. |
| P-203 | A-80 | C-12 | 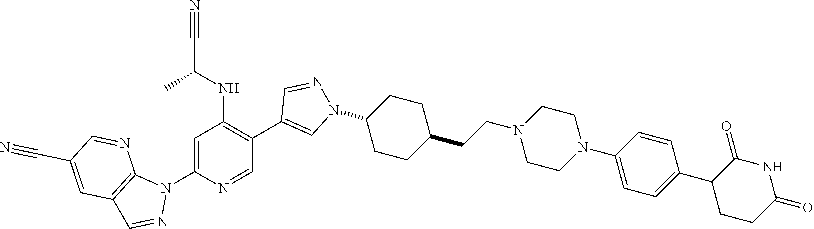 | 14% yield as a brown gum. LCMS method 7: Retention time: 1.614 min, 98.8% purity at 215 nm, [M + H]⁺ = 737.4 ¹H NMR (400 MHz, DMSO-d6): δ ppm 1.15- 1.30 (m, 2H), 1.40-1.52 (m, 1H), 1.62- 1.70 (m, 5H), 1.80-2.04 (m, 5H), 2.12-2.24 (m, 3H), 2.61-2.68 (m, 1H), 2.97-3.02 (m, 2H), 3.16-3.29 (m, 4H), 3.61-3.68 (m, 2H), 3.75-3.89 (m, 3H), 4.24-4.28 (m, 1H), 4.88- 4.94 (m, 1H), 6.60 (d, J = 8.80 Hz, 1H), 6.99 (d, J = 8.80 Hz, 2H), 7.13 (d, J = 8.40 Hz, 2H), 7.44 (s, 1H), 7.81 (s, 1H), 8.15 (s, 1H), 8.27 (s, 1H), 8.69 (s, 1H), 9.04-9.06 (m, 2H), 10.81 (s, 1H). One proton was not apparent by ¹H NMR. |
| P-207 | A66 | C48 | 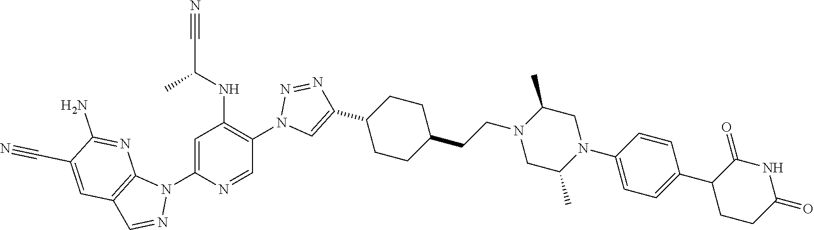 | 26% yield as an off-white solid. LCMS method 7: Retention time: 1.740 min, 99.7% purity at 215 nm, [M + H]⁺ = 781.4 ¹H NMR (400 MHz, DMSO-d6, 80 C): δ ppm 0.98-1.01 (m, 2H), 1.11-1.31 (m, 3H), 1.35 (d, J = 6.4 Hz, 3H), 1.48-1.59 (m, 4H), 1.66- 1.71 (m, 5H), 1.93-1.96 (m, 2H), 2.09-2.33 (m, 4H), 2.62-2.70 (m, 1H), 2.78-2.84 (m, 1H), 2.96-3.02 (m, 1H), 3.11-3.19 (m, 1H), 3.32-3.64 (m, 5H), 3.79-3.85 (m, 2H), 4.96- 5.00 (m, 1H), 6.92-7.21 (m, 7H), 7.96 (s, 1H), 8.22 (s, 1H), 8.29 (s, 1H), 8.37 (s, 1H), 8.53 (s, 1H), 10.50 (s, 1H). |
| P-211 | A-81 | C-12 | 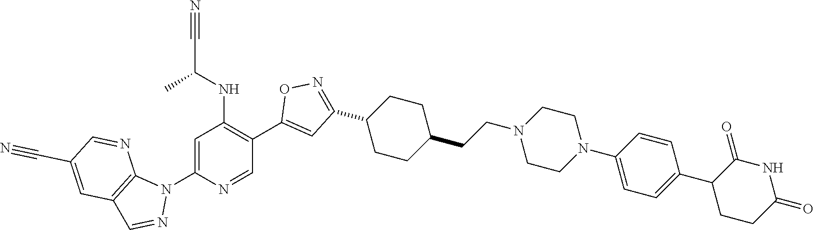 | 7.0% yield as an off-white solid. LCMS method 11: Retention time: 1.962 min, 96.3% purity at 215 nm, (M + H)+ = 738.5 ¹H NMR (400 MHz, DMSO-d6): δ ppm 1.14- 1.24 (m, 2H), 1.39-1.47 (m, 1H), 1.50-1.54 (m, 2H), 1.63-1.70 (m, 5H), 1.87-1.90 (m, 2H), 1.98-2.15 (m, 5H), 2.61-2.69 (m, 1H), 2.75-2.81 (m, 1H), 2.96-3.01 (m, 2H), 3.15- 3.24 (m, 4H), 3.75-3.86 (m, 2H), 5.00- 5.04 (m, 1H), 6.97-7.01 (m, 4H), 7.11-7.14 (m, 2H), 7.60 (s, 1H) , 8.61 (s, 1H), 8.71 (s, 1H), 9.04 (d, J = 2.0 Hz, 1H), 9.06 (d, J = 2.4 Hz, 1H), 10.80 (s, 1H) Three protons were not apparent by ¹H NMR. |
| P-214 | A66 | C25 | 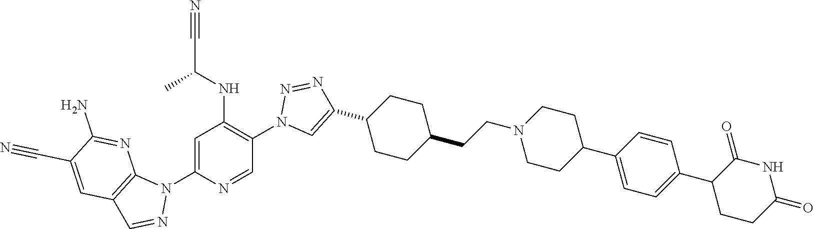 | 14% yield as an off-white solid. LCMS method 7: Retention time: 1.389 min, 98.7% purity at 215 nm, [M + H]⁺ = 752.4 ¹H NMR (400 MHz, DMSO-d6): δ ppm 1.15- 1.23 (m, 2H), 1.39-1.56 (m, 3H), 1.62- 1.68 (m, 5H), 1.85-1.93 (m, 4H), 2.03-2.08 (m, 3H), 2.13-2.21 (m, 3H), 2.68-2.89 (m, 2H), 3.04-3.19 (m, 4H), 3.62-3.65 (m, 2H), 3.83-3.87 (m, 1H), 4.98-5.04 (m, 1H), 7.20 (d, J = 8.8 Hz, 1H), 7.19-7.24 (m, 4H), 7.37 (brs, 2H), 8.00 (s, 1H), 8.25 (s, 1H), 8.33 (s, 1H), 8.35 (s, 1H), 8.59 (s, 1H), 10.84 (s, 1H). Two protons were not apparent by ¹H NMR. |
| P-215 | A66 | C-74 | 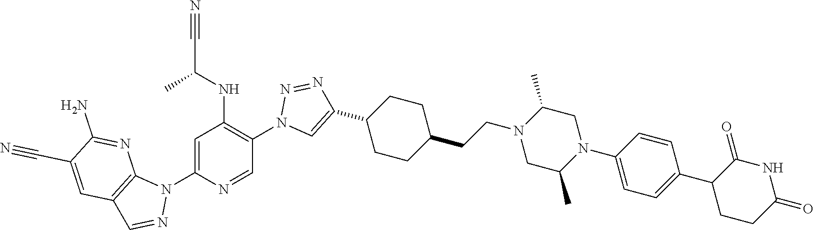 | 42% yield as an off-white solid. LCMS method 7: Retention time: 1.713 min, 99.1% purity at 215 nm, [M + H]⁺ = 781.4 ¹H NMR (400 MHz, DMSO-d6, 80 C): δ ppm 0.96-1.01 (m, 2H), 1.19-1.35 (m, 6H), 1.45- 1.61 (m, 3H), 1.66-1.73 (m, 5H), 1.93- 1.95 (m, 2H), 2.12-2.21 (m, 4H), 2.61-2.69 (m, 1H), 2.76-2.85 (m, 1H), 2.92-3.05 (m, 1H), 3.12-3.42 (m, 5H), 3.76-3.84 (m, 1H), 4.96-5.01 (m, 1H), 6.95-7.22 (m, 7H), 7.96 (S, 1H), 8.22 (s, 1H), 8.29 (s, 1H), 8.38 (s, 1H), 8.53 (s, 1H), 10.50 (s, 1H). Three protons were not apparent by ¹H NMR. |
| P-216 | A66 | C43 | 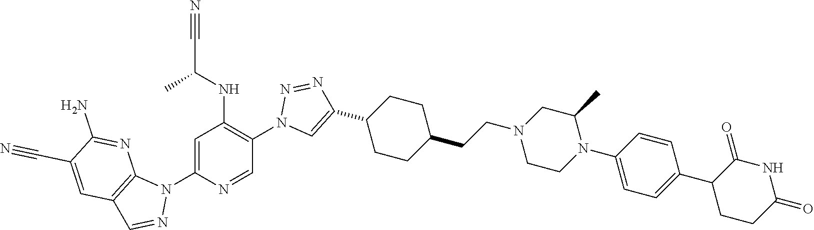 | 19% yield as a brown solid. LCMS method 7: Retention time: 1.352 min, 95.4% purity at 215 nm, [M + H]⁺ = 768.4 ¹H NMR (400 MHz, DMSO-d6, 80 C): δ ppm 1.06 (d, J = 6.0 Hz, 3H), 1.15-1.22 (m, 2H), 1.49-1.56 (m, 5H), 1.67 (d, J = 7.20 Hz, 3H), 1.91-1.94 (m, 2H), 2.08-2.19 (m, 5H), 2.60- 2.78 (m, 3H), 3.20-3.30 (m, 1H), 3.72- 3.78 (m, 1H), 3.88-3.92 (m, 1H), 4.95-4.99 (m, 1H), 6.89-6.96 (m, 5H), 7.10 (d, J = 8.0 Hz, 2H), 7.94 (s, 1H), 8.22 (s, 1H), 8.27 (s, 1H), 8.37 (s, 1H), 8.52 (s, 1H), 10.42 (s, 1H). Six protons were not apparent by ¹H NMR. |
| P-217 | A-82 | C12 | 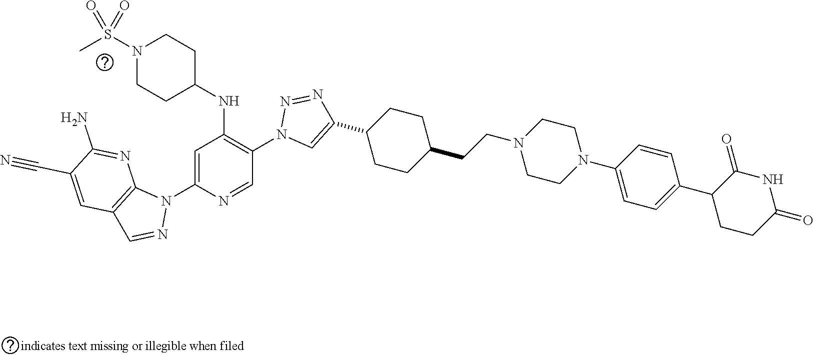 | 17 % yield as an off-white solid. LCMS method 6: Retention time: 1.999 min, 99.1% purity at 215 nm, [M + H]⁺ = 861.5 ¹H-NMR (400 MHz, DMSO-d6): δ ppm 1.13- 1.25 (m, 2H), 1.38-1.69 (m, 8H), 1.87-1.91 (m, 2H), 1.98-2.18 (m, 6H), 2.72-2.81 (m, 1H), 2.88 (s, 3H), 2.96-3.02 (m, 4H), 3.12- 3.26 (m, 4H), 3.54-3.61 (m, 2H), 3.83-3.86 (m, 2H), 6.46 (d, J = 6.6 Hz, 1H), 6.99 (d, J = 8.80 Hz, 2H), 7.13 (d, J = 8.80 Hz, 2H), 7.26 (brs, 2H), 7.54 (s, 1H), 8.22 (s, 1H), 8.29 (s, 1H), 8.39 (s, 1H), 8.57 (s, 1H), 10.79 (s, 1H). Five protons were not apparent by ¹H NMR. |
| P-219 | A66 | C44 | 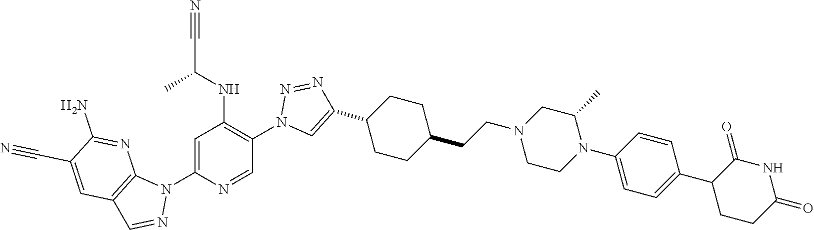 | 27 % yield as an off-white solid. LCMS method 7: Retention time: 1.650 min, 95.4% purity at 215 nm, [M + H]⁺ = 767.3 ¹H NMR (400 MHz, DMSO-d6, 80 C): δ ppm 1.02-1.06 (m, 3H), 1.18-1.21 (m, 2H), 1.41- 1.58 (m, 3H), 1.64-1.72 (m, 5H), 1.88- 1.91 (m, 2H), 2.05-2.18 (m, 4H), 2.62-2.81 (m, 2H), 3.07-3.24 (m, 6H), 3.78-3.83 (m, 1H), 4.94-4.98 (m, 1H), 6.97-7.21 (m, 7H), 7.95 (s, 1H), 8.22 (s, 1H), 8.28 (s, 1H), 8.35 (s, 1H), 8.53 (s, 1H), 10.54 (s, 1H). Four protons were not apparent by ¹H NMR. |
| P-220 | A-66 | C-44 | 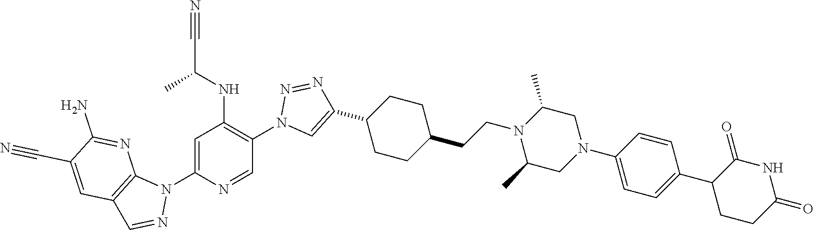 | 10% yield as an off-white solid. LCMS method 7: Retention time: 1.794 min, 97.9% purity at 215 nm, [M + H]⁺ = 781.3 ¹H NMR (400 MHz, DMSO-d6): δ ppm 1.13- 1.25 (m, 2H), 1.36-1.39 (m, 6H), 1.45- 1.61 (m, 4H), 1.62-1.64 (m, 4H), 1.90-2.03 (m, 3H), 2.13-2.17 (m, 3H), 2.65-2.67 (m, 1H), 2.72-2.84 (m, 2H), 3.01-3.18 (m, 2H), 3.32-3.39 (m, 1H), 3.74-3.91 (m, 3H), 4.98- 5.03 (m, 1H), 6.96-6.98 (m, 2H), 7.08- 7.12 (m, 3H), 7.37 (brs, 2H), 7.99 (s, 1H), 8.25 (s, 1H), 8.32 (s, 1H), 8.34 (s, 1H), 8.58 (s, 1H), 10.79 (s, 1H). Three protons were not apparent by ¹H NMR. |
| P-222 | A-66 | C-73 | 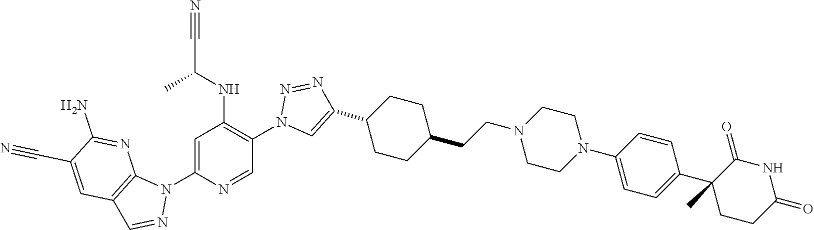 | 24% yield as a pale yellow solid. LCMS method 7: Retention time: 1.380 min, 95.0% purity at 215 nm, [M + H]⁺ = 767.2 ¹H NMR (400 MHz, DMSO-d6): δ ppm 1.12- 1.15 (m, 2H), 1.39-1.50 (m, 8H), 1.63 (d, J = 6.80 Hz, 3H), 1.86-1.90 (m, 2H), 2.04- 2.15 (m, 4H), 2.33-2.44 (m, 4H), 2.65-2.75 (m, 1H), 3.11-3.14 (m, 4H), 4.98-5.00 (m, 1H), 6.92 (d, J = 8.80 Hz, 2H), 7.10-7.12 (m, 3H), 7.37 (brs, 2H), 7.97 (s, 1H), 8.25 (s, 1H), 8.32-8.36 (m, 2H), 8.58 (s, 1H), 10.84 (s, 1H). Four protons were not apparent by ¹H NMR. |
| P-223 | A-66 | C-73 | 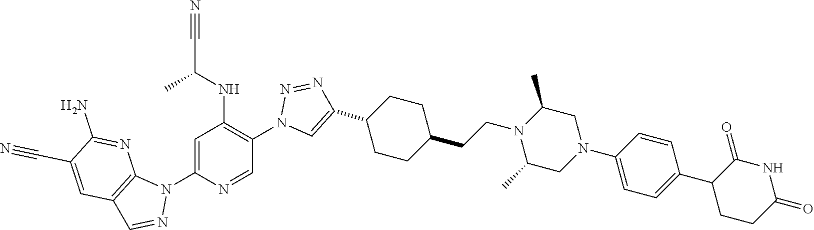 | 25% yield as a pale brown solid. LCMS method 7: Retention time: 1.754 min, 96.4% purity at 215 nm, [M + H]⁺ = 781.4 ¹H NMR (400 MHz, DMSO-d6): δ ppm 1.15- 1.30 (m, 3H), 1.37-1.40 (m, 6H), 1.41- 1.68 (m, 8H), 1.91-2.06 (m, 4H), 2.11-2.23 (m, 4H), 2.72-2.85 (m, 2H), 3.03-3.20 (m, 2H), 3.32-3.40 (m, 1H), 3.82-3.94 (m, 2H), 4.97-5.05 (m, 1H), 6.98 (d, J = 8.8 Hz, 2H), 7.09-7.13 (m, 3H), 7.38 (brs, 2H), 8.01 (s, 1H), 8.26 (s, 1H), 8.33 (s, 1H), 8.35 (s, 1H), 8.59 (s, 1H), 10.80 (s, 1H). Two protons were not apparent by ¹H NMR. |
| P-224 | A-84 | C-12 | 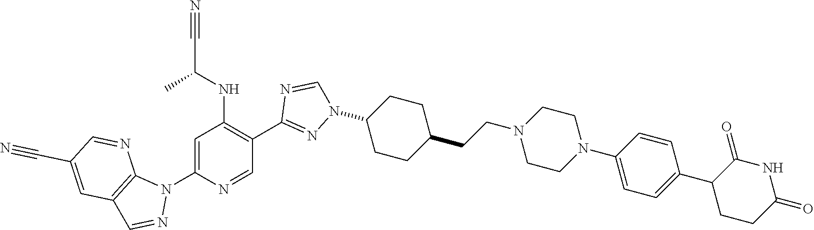 | 5.2% yield as an off-white solid. LCMS method 8: Retention time: 2.005 min, 99.8% purity at 215 nm, [M + H]⁺ = 738.3 ¹H NMR (400 MHz, DMSO-d6): δ ppm 1.15- 1.30 (m, 2H), 1.40-1.55 (m, 1H), 1.61- 1.65 (m, 2H), 1.76 (d, J = 6.80 Hz, 3H), 1.83- 2.04 (m, 5H), 2.10-2.29 (m, 3H), 2.67-2.68 (m, 1H), 2.97-3.02 (m, 2H), 3.11-3.27 (m, 4H), 3.61-3.87 (m, 5H), 4.37-4.48 (m, 1H), 4.99-5.12 (m, 1H), 6.99 (d, J = 8.8 Hz, 2H), 7.13 (d, J = 8.4 Hz, 2H), 7.57 (s, 1H), 8.63 (d, J = 7.2 Hz, 1H), 8.70 (s, 1H), 8.85 (s, 1H), 9.05-9.07 (m, 3H), 10.80 (s, 1H). One proton was not apparent by ¹H NMR. |
| P-242 | A-66 | C-81 | 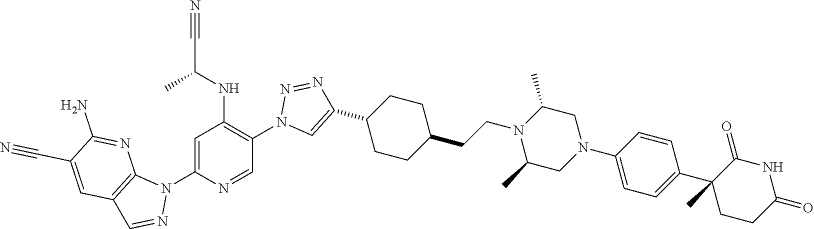 | 12% yield as a pale yellow solid. LCMS method 11: Retention time: 2.300 min, 92.3% purity at 215 nm, [M + H]⁺ = 795.4 ¹H NMR (400 MHz, DMSO-d6) δ ppm 1.15- 1.25 (m, 2H), 1.35-1.42 (m, 9H), 1.45-1.55 (m, 5H), 1.63 (d, J = 6.8 Hz, 4H), 1.90-1.93 (m, 2H), 2.04-2.05 (m, 2H), 2.11-2.17 (m, 2H), 2.35-2.49 (m, 2H), 2.73-2.86 (m, 2H), 3.02-3.18 (m, 2H), 3.62-3.65 (m, 2H), 3.75- 3.90 (m, 2H), 4.98-5.02 (m, 1H), 7.0 (d, J = 9.2 Hz, 2H), 7.08-7.17 (m, 3H), 7.36 (brs, 2H), 7.99-8.02 (m, 1H), 8.25 (s, 1H), 8.29- 8.34 (m, 2H), 8.58 (s, 1H), 10.87 (s, 1H). |
| P-245 | A-66 | C-80 | 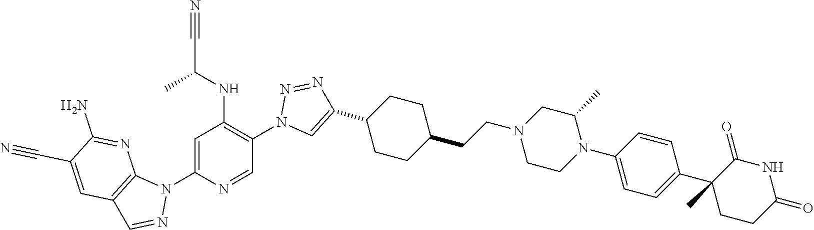 | 16% yield as a pale yellow solid. LCMS method 7: Retention time: 1.775 min, 96.3% purity at 215 nm, [M + H]⁺ = 781.4 ¹H NMR (400 MHz, DMSO-d6): δ ppm 0.92 and 1.10 (d, J = 6.4 Hz, 3H), 1.18-1.24 (m, 2H), 1.40-1.54 (m, 6H), 1.62-1.68 (m, 5H), 1.87-1.91 (m, 2H), 2.06-2.21 (m, 4H), 2.74- 2.78 (m, 1H), 3.05-3.31 (m, 4H), 3.51- 3.69 (m, 2H), 4.98-5.02 (m, 1H), 6.95 (d, J = 9.2 Hz, 2H), 7.10-7.38 (m, 3H), 7.45 (brs, 2H), 8.01 (s, 1H), 8.26 (s, 1H), 8.32-8.40 (m, 2H), 8.58 (s, 1H), 10.87 and 10.93 (s, 1H). Five protons were not apparent by ¹H NMR. |

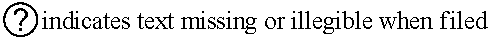

General Procedure X-16. The scheme shown below for the synthesis of P-199 is provided as a representative synthesis for General Procedure X-16.

### Example S56. 1-(5-(4-((1R,4R)-4-((1R)-1-chloro-2-(4-(4-(2,6-dioxopiperidin-3-yl) phenyl) piperazin-1-yl) ethyl) cyclohexyl)-1H-1,2,3-triazol-1-yl)-4-(((R)-1-cyanoethyl) amino) pyridin-2-yl)-1H-pyrazolo[3,4-b]pyridine-5-carbonitrile.TFA (P-199)

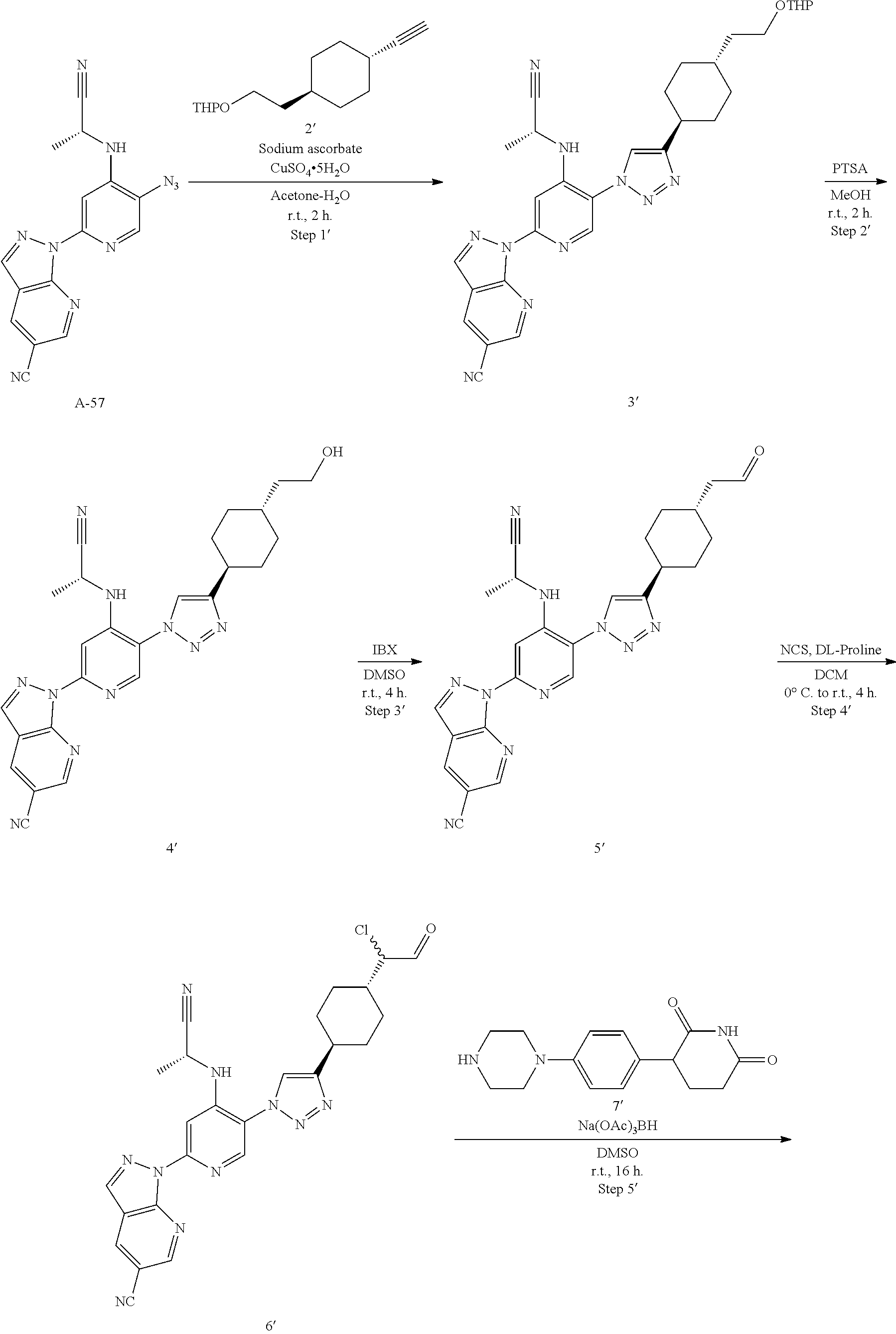

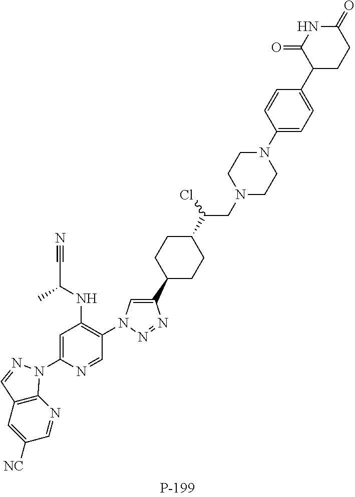 [SMILES: C[C@H](C#N)Nc1cc(-n2ncc3cc(C#N)cnc32)ncc1-n1cc([C@H]2CC[C@H](C(Cl)CN3CCN(c4ccc(C5CCC(=O)NC5=O)cc4)CC3)CC2)nn1]

UPLC Method 1. Aquity BEH-C18, 50×3.0 mm, 1.7 Temperature: RT, Flow: 1.0 mL/min, Run Time: 5 minutes, Mobile Phase Conditions: Mobile Phase-A: 0.1% TFA in H₂O, Mobile Phase-B: 0.1% TFA in ACN, Gradient: Initial 80% Mobile Phase A and 20% Mobile Phase B linear gradient to 100% Mobile Phase B for 3.0 min. MSD positive.

LCMS Method 2. Kinetex XB-C18, 75×3.0 mm, 2.6 Temperature: RT, Flow: 1.0 mL/min, Run Time: 5 minutes, Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 100% Mobile Phase B for 4.0 min. MSD positive.

LCMS Method 3. X-SELECT, 150×4.6 mm, 3.5 Temperature: RT, Flow: 1.0 mL/min, Run Time: 25 minutes, Mobile Phase Conditions: Mobile Phase-A: 5.0 mm Ammonium formate Buffer:ACN (98:2), Mobile Phase-B: 5.0 mm ACN:buffer(98:2), Gradient: Initial 90% Mobile Phase A and 10% Mobile Phase B linear gradient to 100% Mobile Phase B for 18.0 min. MSD positive.

Step 1′. Preparation of 1-(4-(((R)-1-cyanoethyl)amino)-5-(4-01r,4R)-4-(2-((tetrahydro-2H-pyran-2-yl)oxy)ethyl)cyclohexyl)-1H-1,2,3-triazol-1-yl)469yridine-2-yl)-1H-pyrazolo[3,4-b]pyridine-5-carbonitrile (3′): To a stirred solution of (R)-1-(5-azido-4-((1-cyanoethyl)amino)469yridine-2-yl)-1H-pyrazolo[3,4-b]pyridine-5-carbonitrile A-57 (19.0 g, 41.4 mmol) in acetone (180.0 mL) was added 2-(2-((1r,4r)-4-ethynylcyclohexyl)ethoxy)tetrahydro-2H-pyran 2′ (10.44 g, 14.37 mmol) followed by sodium ascorbate (3.28 g, 16.57 mmol) and then a solution of copper(II) sulphate pentahydrate (4.14 g, 16.57 mmol) in H₂O (20.0 mL) was added at room temperature. The reaction mixture was stirred at room temperature for 2 h. The reaction mixture was treated with water and extracted with ethyl acetate (3×500 mL). The combined organic layer was dried over sodium sulphate and concentrated under reduced pressure to give the crude product 1-(4-((®-1-cyanoethyl)amino)-5-(4-((1r,4R)-4-(2-((tetrahydro-2H-pyran-2-yl)oxy)ethyl)cyclohexyl)-1H-1,2,3-triazol-1-yl)469yridine-2-yl)-1H-pyrazolo[3,4-b]pyridine-5-carbonitrile 3′ (25 g) as brown solid, which was used in the next step without further purification.

LCMS method 1: retention time 2.26 min, 75.34% purity at 220 nm, [M+H]⁺=567.2

Step 2′. Preparation of 1-(4-(((R)-1-cyanoethyl)amino)-5-(4-((1r,4R)-4-(2-hydroxyethyl)cyclohexyl)-1H-1,2,3-triazol-1-yl)pyridin-2-yl)-1H-pyrazolo[3,4-b]pyridine-5-carbonitrile (4′): To a stirred solution of 1-(4-(((R)-1-cyanoethyl)amino)-5-(4-((1r,4R)-4-(2-((tetrahydro-2H-pyran-2-yl)oxy)ethyl)cyclohexyl)-1H-1,2,3-triazol-1-yl)pyridin-2-yl)-1H-pyrazolo[3,4-b]pyridine-5-carbonitrile 3′ (25.0 g, 30.0 mmol) in MeOH (20.0 mL), pTSA (1.712 g, 9.0 mmol) was added at RT and stirred for 2 h. The reaction mixture was concentrated under reduced pressure then diluted with water and extracted with DCM (2×100 mL). The organic layer was washed with brine, dried over sodium sulphate, and concentrated under reduced pressure to give crude product 1-(4-(((R)-1-cyanoethyl)amino)-5-(4-((1r,4R)-4-(2-hydroxyethyl)cyclohexyl)-1H-1,2,3-triazol-1-yl)pyridin-2-yl)-1H-pyrazolo[3,4-b]pyridine-5-carbonitrile 4′ (16.0 g) as yellow solid, which was used in the next step without further pufication.

LCMS method 2: retention time 1.742 min, 86.91% purity at 220 nm, [M+H]⁺=483.2

Step 3′. Preparation of 1-(4-(((R)-1-cyanoethyl) amino)-5-(4-((1r,4R)-4-(2-oxoethyl) cyclohexyl)-1H-1,2,3-triazol-1-yl) pyridin-2-yl)-1H-pyrazolo[3,4-b]pyridine-5-carbonitrile (5′): IBX (296 mg, 1.057 mmol, 3.0 eq.) was added to a solution of 1-(4-(((R)-1-cyanoethyl) amino)-5-(4-((1r,4R)-4-(2-hydroxyethyl) cyclohexyl)-1H-1,2,3-triazol-1-yl) pyridin-2-yl)-1H-pyrazolo[3,4-b]pyridine-5-carbonitrile 4′ (200 mg, 0.352 mmol, 1.0 eq.) in DMSO (3 mL, 0.117 M) at RT. The resulting solution was stirred for 4 h at RT. The reaction mixture was diluted ethyl acetate (15 mL) and washed with ice-cold water, aqueous sodium bicarbonate solution followed by brine solution, dried over sodium sulphate, and concentrated under reduced pressure to give the crude product 1-(4-(((R)-1-cyanoethyl) amino)-5-(4-((1r,4R)-4-(2-oxoethyl) cyclohexyl)-1H-1,2,3-triazol-1-yl) pyridin-2-yl)-1H-pyrazolo[3,4-b]pyridine-5-carbonitrile 5′ (180 mg, 78% yield) as pale yellow solid, which was used in the next step without further purification.

LCMS method 2: retention time: 1.926 min, 73.14% purity at 220 nm, [M+H]⁺=481.4

Step 4′. Preparation of 1-(5-(4-((1R,4R)-4-((R)-1-chloro-2-oxoethyl) cyclohexyl)-1H-1,2,3-triazol-1-yl)-4-4(R)-1-cyanoethyl) amino) pyridin-2-yl)-1H-pyrazolo[3,4-b] pyridine-5-carbonitrile (6′): NCS (118 mg, 0.883 mmol, 1.3 eq.) was added to a mixture of 1-(4-(((R)-1-cyanoethyl) amino)-5-(4-((1r,4R)-4-(2-oxoethyl) cyclohexyl)-1H-1,2,3-triazol-1-yl) pyridin-2-yl)-1H-pyrazolo[3,4-b]pyridine-5-carbonitrile 5′ (440 mg, 0.679 mmol, 1.0 eq.) and DL Proline (7.82 mg, 0.068 mmol, 0.1 eq.) in DCM (6 mL, 0.113 M) at 0° C. Reaction mixture was allowed to warm to ambient temperature and stirred at RT for 4 h. The reaction mixture was diluted with DCM (10 mL) and washed with water (2×10 mL), brine solution, dried over sodium sulphate, and concentrated under reduced pressure to give the crude product 1-(5-(4-((1R,4R)-4-((R)-1-chloro-2-oxoethyl) cyclohexyl)-1H-1,2,3-triazol-1-yl)-4-(((R)-1-cyanoethyl) amino) pyridin-2-yl)-1H-pyrazolo[3,4-b]pyridine-5-carbonitrile 6′ (510 mg, 62.3% yield) as pale yellow semi-solid, which was used in the next step without further purification.

LCMS method 2: retention time: 2.377 min, [M+H]⁺=515.2

Step 5′. Preparation of 1-(5-(4-((1R,4R)-4-41R)-1-chloro-2-(4-(4-(2,6-dioxopiperidin-3-yl) phenyl) piperazin-1-yl) ethyl) cyclohexyl)-1H-1,2,3-triazol-1-yl)-4-(((R)-1-cyanoethyl) amino) pyridin-2-yl)-1H-pyrazolo[3,4-b]pyridine-5-carbonitrile.TFA (P-199): 1-(5-(441R,4R)-4-((R)-1-chloro-2-oxoethyl) cyclohexyl)-1H-1,2,3-triazol-1-yl)-4-(((R)-1-cyanoethyl) amino) pyridin-2-yl)-1H-pyrazolo[3,4-b]pyridine-5-carbonitrile 6′ (510 mg, 0.423 mmol, 1 eq.) and 3-(4-(piperazin-1-yl) phenyl) piperidine-2,6-dione 7′ (139 mg, 0.508 mmol, 1.2 eq.) were mixed in DMSO (5.0 mL) and stirred at RT for 30 min. Sodium triacetoxyborohydride (179 mg, 0.846 mmol, 2.0 eq.) was then added and allowed to stir at RT for 16 h. The reaction mixture was quenched with water (0.5 mL) and purified by preparative HPLC to give 1-(5-(4-((1R,4R)-4-((1R)-1-chloro-2-(4-(4-(2,6-dioxopiperidin-3-yl) phenyl) piperazin-1-yl) ethyl) cyclohexyl)-1H-1,2,3-triazol-1-yl)-4-(((R)-1-cyanoethyl) amino) pyridin-2-yl)-1H-pyrazolo[3,4-b]pyridine-5-carbonitrile.TFA P-199 (22 mg, 0.023 mmol, 5.52% yield) as a pale yellow solid.

LCMS method 3: retention time: 13.76 min, 94.17% purity at 215 nm, [M+H]⁺=772.4.

¹H NMR (400 MHz, DMSO-d6): δ ppm 1.41-1.60 (m, 5H), 1.61 (d, J=6.80 Hz, 3H), 1.89-2.03 (m, 4H), 2.16-2.33 (m, 4H), 2.61-2.64 (m, 1H), 2.68-2.77 (m, 1H), 3.08-3.17 (m, 2H), 3.70-3.84 (m, 8H), 4.59-4.61 (m, 1H), 5.02-5.04 (m, 1H), 6.96-7.00 (m, 2H), 7.08-7.14 (m, 2H), 7.19-7.21 (m, 1H), 7.68 (s, 1H), 8.39-8.43 (m, 2H), 8.74 (d, J=6.8 Hz, 1H), 9.07-9.08 (m, 2H), 10.81 (s, 1H).

Table 45 summarizes the compounds prepared using General Procedure X-16.

TABLE 45
Final Compounds Prepared via General Procedure X-16

| Compound | TMB | CBM | |
|---|---|---|---|
| No. | Portion | Portion | Structure |
| P-198 | A-57 | C-12 | 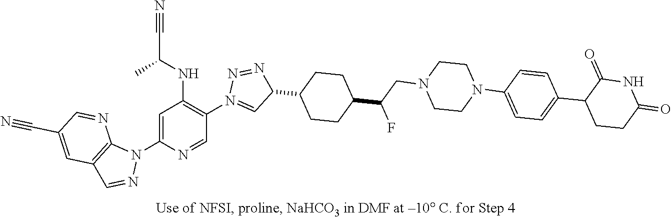 |
| P-199 | A-57 | C-12 | 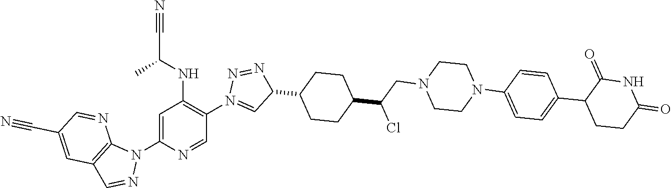 |
| P-213 | A-57 | C-12 | 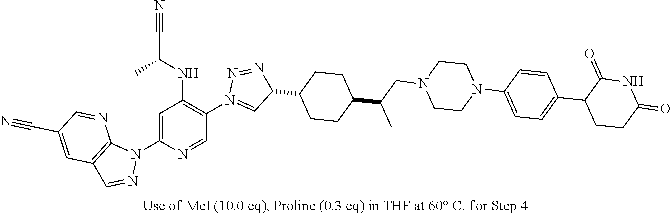 |

| Compound | |
|---|---|
| No. | Characterization |
| P-198 | 45% yield as an off-white solid. |
| | LCMS method 7: Retention time: 1.357 min, 93.0% purity at |
| | 215 nm, [M + H]⁺ = 756.2 |
| | ¹H NMR NMR (400 MHz, DMSO-d6): δ ppm 1.28-1.40 (m, 2H), |
| | 1.47-1.54 (m, 2H), 1.61 (d, J = 6.8 Hz, 3H), 1.64-1.75 (m, 1H), |
| | 1.82-1.85 (m, 1H), 1.94-2.03 (m, 2H), 2.18-2.21 (m, 3H), 2.44- |
| | 2.49 (m, 1H), 2.60-2.68 (m, 5H), 2.71-2.75 (m, 1H), 3.11-3.16 |
| | (m, 4H), 3.73-3.74 (m, 1H), 4.52-4.65 (m, 1H), 4.94-5.05 (m, |
| | 1H), 6.91 (d, J = 8.80 Hz, 2H), 7.06 (d, J = 8.80 Hz, 2H), 7.20 |
| | (d, J = 8.40 Hz, 1H), 7.67 (s, 1H), 8.38 (s, 1H), 8.41 (s, 1H), |
| | 8.73 (s, 1H), 9.06-9.08 (m, 2H), 10.79 (s, 1H). |
| | ¹⁹F NMR (400 MHz, DMSO-d6): δ ppm −184.6. |
| | Two protons were not apparent by ¹H NMR. |
| P-199 | 5.5% yield as a pale yellow solid. |
| | LCMS method 12: Retention time: 13.76 min, 94.2% purity at |
| | 215 nm, [M + H]⁺ = 772.4 |
| | ¹H NMR (400 MHz, DMSO-d6): δ ppm 1.41-1.60 (m, 5H), |
| | 1.61 (d, J = 6.80 Hz, 3H), 1.89-2.03 (m, 4H), 2.16-2.33 (m, |
| | 4H), 2.61-2.64 (m, 1H), 2.68-2.77 (m, 1H), 3.08-3.17 (m, |
| | 2H), 3.70-3.84 (m, 8H), 4.59-4.61 (m, 1H), 5.02-5.04 (m, |
| | 1H), 6.96-7.00 (m, 2H), 7.08-7.14 (m, 2H), 7.19-7.21 (m, |
| | 1H), 7.68 (s, 1H), 8.39-8.43 (m, 2H), 8.74 (d, J = 6.8 Hz, 1H), |
| | 9.07-9.08 (m, 2H), 10.81 (s, 1H). |
| P-213 | 32.3% yield as an off-white solid. |
| | LCMS method 7: Retention time: 1.415 min, 98.0% purity at |
| | 215 nm, [M + H]⁺ = 752.3 |
| | 1H-NMR (400 MHz, DMSO-d6): δ ppm 1.02 (d, J = 6.80 Hz, |
| | 3H), 1.22-1.60 (m, 5H), 1.61 (d, J = 6.80 Hz, 3H), 1.78-1.81 |
| | (m, 1H), 1.92-2.03 (m, 2H), 2.15-2.21 (m, 3H), 2.66-2.75 |
| | (m, 1H), 3.09-3.27 (m, 7H), 3.60-3.68 (m, 2H), 3.75-3.84 |
| | (m, 4H), 5.01-5.05 (m, 1H), 6.98-7.00 (m, 2H), 7.10-7.14 |
| | (m, 2H), 7.20-7.23 (m, 1H), 7.68 (s, 1H), 8.37 (s, 1H), 8.41 (s, |
| | 1H), 8.73 (s, 1H), 9.06-9.11 (m, 2H), 10.81 (s, 1H). |
| | One proton was not apparent by ¹H NMR. |

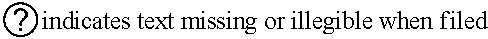

General Procedure X-17. The scheme shown below for the synthesis of P-202 is provided as a representative synthesis for General Procedure X-17.

### Example S57. 1-(4-(((R)-1-cyanoethyl)amino)-5-(4-(4-(2-(4-(4-(2,6-dioxopiperidin-3-yl)phenyl)piperazin-1-yl)ethyl)bicyclo[2.2.1]heptan-1-yl)-1H-1,2,3-triazol-1-yl)pyridin-2-yl)-1H-pyrazolo[3,4-b]pyridine-5-carbonitrile, TFA (P-202)

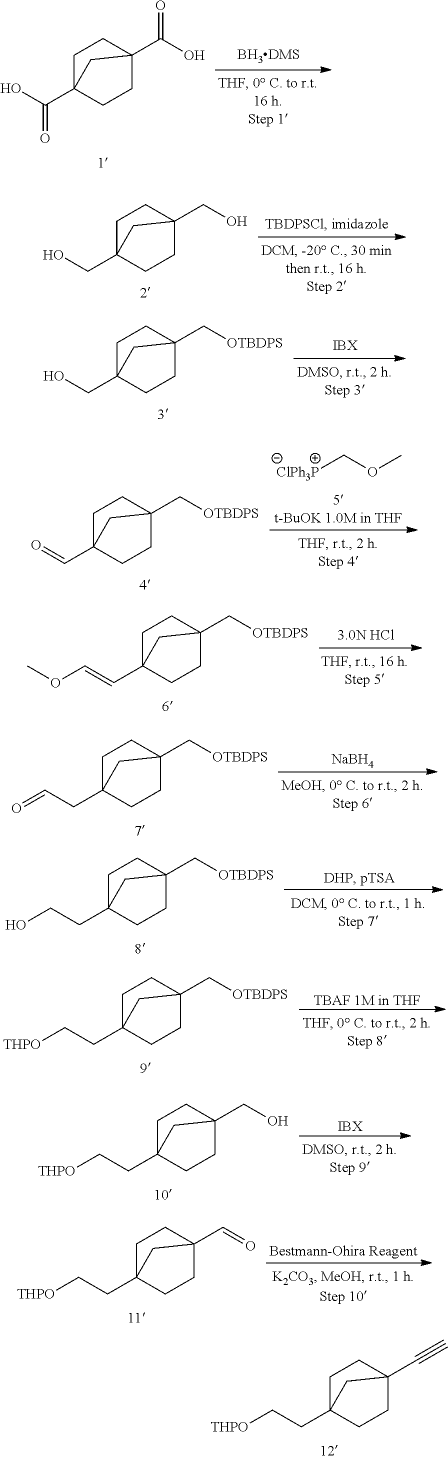

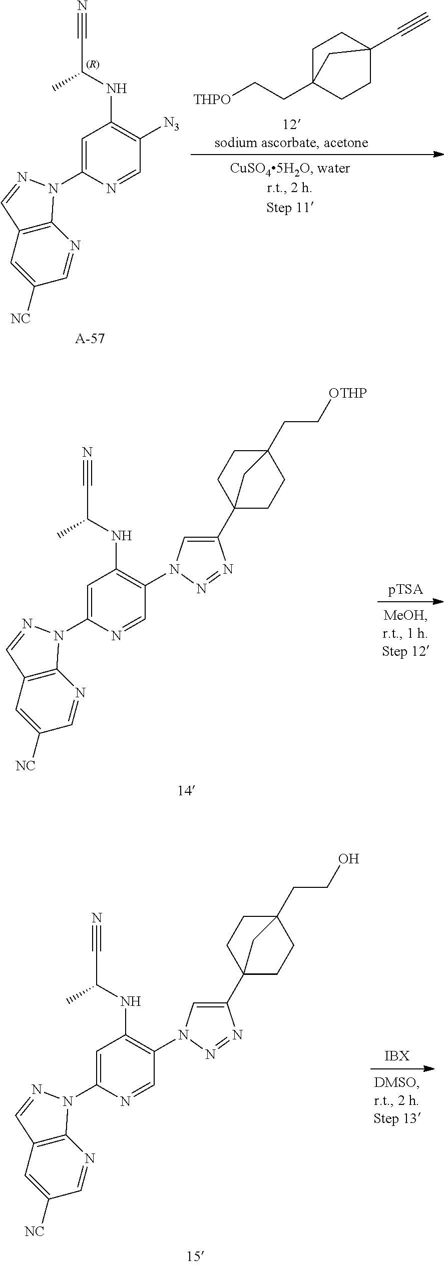

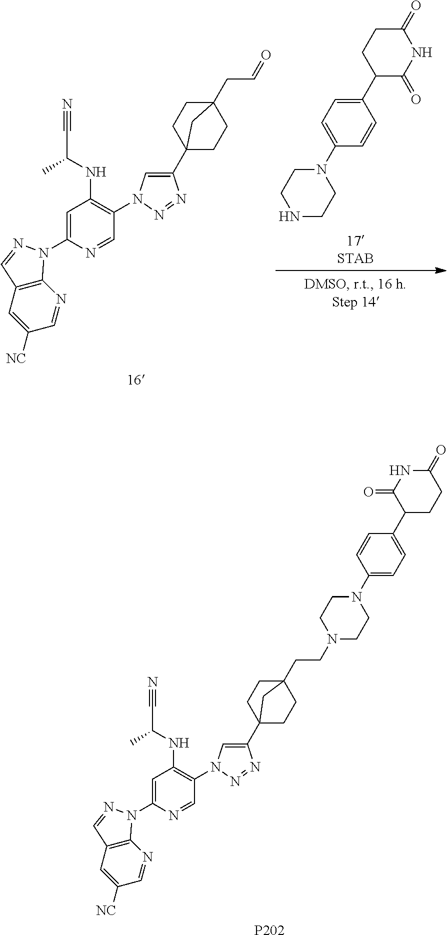 [SMILES: C[C@H](C#N)Nc1cc(-n2ncc3cc(C#N)cnc32)ncc1-n1cc(C23CCC(CC=O)(CC2)C3)nn1 ; O=C1CCC(c2ccc(N3CCNCC3)cc2)C(=O)N1 ; "P202" ; C[C@H](C#N)Nc1cc(-n2ncc3cc(C#N)cnc32)ncc1-n1cc(C23CCC(CCN4CCN(c5ccc(C6CCC(=O)NC6=O)cc5)CC4)(CC2)C3)nn1]

UPLC Method 1. Column: Aquity BEH C18, 50×2.1 mm, 1.7 Temperature: 45° C., Flow: 1.0 mL/min, run time: 4.2 min. Mobile Phase-A: 0.1% TFA in Water, Mobile Phase-B: 0.1% TFA in CH₃CN, Gradient: Initial 80% Mobile Phase A and 20% Mobile Phase B linear gradient to 100% Mobile Phase B for 3.0 min. MSD positive.

LCMS Method 2. Column: Kinetex XB-C18, 75×3.0 mm, 2.6 μm, Temperature: ° C., Flow: 1.0 mL/min, run time: 5.0 min. Mobile Phase-A: 5.0 mm Ammonium formate pH 3.3:CH₃CN (98:02), Mobile Phase-B: CH₃CN: Buffer (98:02), Gradient: Initial 80% Mobile Phase A and 20% Mobile Phase B linear gradient to 100% Mobile Phase B for 4.0 min. MSD positive.

Step 1′. Preparation of bicyclo[2.2.1]heptane-1,4-diyldimethanol 2′: To a stirred suspension of bicyclo[2.2.1]heptane-1,4-dicarboxylic acid 1′ (5.5 g, 29.9 mmol, 1.0 eq) in THF (100 mL, 0.2 M) was added boranemethylsulfide complex (59.7 mL, 119 mmol, 4.0 eq) at 0° C. and the resulting mixture was stirred at room temperature for 16 h. The reaction mixture was quenched with MeOH (100 mL) at 0° C., and the reaction mixture was concentrated under reduced pressure to afford the crude product. The crude product was purified by column chromatography using silica gel (230-400 mesh) with 80% ethyl acetate/pet ether to afford bicyclo[2.2.1]heptane-1,4-diyldimethanol 2′ (4.65 g, 100% yield) as a colorless liquid.

¹H NMR (400 MHz, DMSO-d6): δ ppm 1.05 (s, 2H), 1.18-1.22 (m, 4H), 1.50-1.51 (m, 4H), 3.41 (s, 4H), 4.36 (brs, 2H).

Step 2′. (4-(((tert-butyldiphenylsilyl)oxy)methyl)bicyclo[2.2.1]heptan-1-yl)methanol 3′: To a stirred suspension of bicyclo[2.2.1]heptane-1,4-diyldimethanol 2′ (4.5 g, 28.8 mmol, 1.0 eq.) in anhydrous DCM (450 mL) was added imidazole (1.961 g, 28.8 mmol, 1.0 eq.) at −20° C. followed by TBDPSCl (6.66 mL, 25.9 mmol, 0.9 eq.) in DCM (100 mL). Then the reaction mixture was stirred at the same temperature for 30 min and then slowly warmed to room temperature and stirred for another 16 h. The reaction mixture was quenched with water (100 mL) and extracted with DCM (2×150 mL). The organic phases were combined and washed with brine (100 mL), combined organic phases were dried over sodium sulfate, filtered, and the filtrate was concentrated under reduced pressure to give a crude product. The crude product was purified by column chromatography using silica gel (230-400 mesh) eluted with 60% ethyl acetate/pet ether to afford (4-(((tertbutyldiphenylsilyl)oxy)methyl)bicyclo[2.2.1]heptan-1-yl)methanol 3′ (3.1 g, 27.3% yield) as a colorless liquid.

¹H NMR (400 MHz, DMSO-d6): δ ppm 1.01 (s, 9H), 1.16-1.20 (m, 2H), 1.24-1.33 (m, 4H), 1.53-1.58 (m, 4H), 3.43 (d, J=5.2 Hz, 2H), 3.66 (s, 2H), 4.40 (t, J=5.2 Hz, 1H), 7.41-7.47 (m, 6H), 7.59-7.62 (m, 4H).

Step 3′. 4-(((tert-butyldiphenylsilyl)oxy)methyl)bicyclo[2.2.1]heptane-1-carbaldehyde 4′: To a stirred solution of (4-(((tertbutyldiphenylsilyl)oxy)methyl)bicyclo[2.2.1]heptan-1-yl)methanol 3′ (2.9 g, 7.35 mmol, 1.0 eq.) in anhydrous DMSO (30 mL) was added IBX (4.12 g, 14.70 mmol, 2.0 eq.) under nitrogen atmosphere. The resulting mixture was stirred at room temperature for 2 h. Then, water (150 mL) was added to the reaction mixture and extracted with ethyl Acetate (2×150 mL). The combined organic phases were washed with saturated sodium bicarbonate solution (150 mL), dried over anhydrous sodium sulfate, filtered, and the filtrate was concentrated under reduced pressure to afford 4-(((tertbutyldiphenylsilyl)oxy)methyl)bicyclo[2.2.1]heptane-1-carbaldehyde 4′ (3.0 g) as a colorless liquid, which was used in the next step without further purification.

¹H NMR (400 MHz, CDCl₃): δ ppm 1.07 (s, 9H), 1.38-1.77 (m, 10H), 3.72 (s, 2H), 7.38-7.45 (m, 6H), 7.65-7.68 (m, 4H), 9.84 (s, 1H).

Step 4′. tert-butyl((4-(2-methoxyvinyl)bicyclo[2.2.1]heptan-1-yl)methoxy)diphenylsilane 6′: To a stirred suspension of (methoxymethyl)triphenylphosphonium chloride salt 5′ (7.86 g, 22.92 mmol, 3.0 eq.) in anhydrous THF (10 mL) was added 1.0 M potassium tert-butoxide in THF (30.6 mL, 30.6 mmol, 4.0 eq.) under nitrogen atmosphere at room temperature and the resulting mixture was stirred at room temperature for 5 min. Then, 4-(((tertbutyldiphenylsilyl)oxy)methyl)bicyclo[2.2.1]heptane-1-carbaldehyde 4′ (3 g, 7.64 mmol, 1.0 eq.) in THF (10 mL) was added into the reaction mixture and stirred at room temperature for another 2 h. The reaction mixture was quenched with ice-cold water (150 mL) and extracted with ethyl acetate (2×150 mL). The combined organic phases were washed with brine (100 mL), dried over sodium sulfate, and filtered, and the filtrate was concentrated under reduced pressure to give a crude product. The crude product was purified by column chromatography using silica-gel (230-400 mesh) with 20% ethyl acetate/pet ether to afford (E)-tert-butyl((4-(2-methoxyvinyl)bicyclo[2.2.1]heptan-1-yl)methoxy)diphenylsilane 6′ (2.4 g) as a colorless liquid, which was used in the next step without further purification.

Step 5′. 2-(4-(((tert-butyldiphenylsilyl)oxy)methyl)bicyclo[2.2.1]heptan-1-yl)acetaldehyde 7′: To a stirred solution of (E)-tert-butyl((4-(2-methoxyvinyl)bicyclo[2.2.1]heptan-1-yl)methoxy)diphenylsilane 6′ (2.4 g, 5.71 mmol, 1.0 eq.) in THF (20 mL) was added 3 N HCl (20 mL, 60.0 mmol, 10.0 eq.) at room temperature and the resulting mixture was stirred at room temperature for 16 h. Then, water (100 mL) was added to the reaction mixture and extracted with ethyl acetate (2×150 mL), the combined organic extracts were washed with brine (100 mL), dried over sodium sulfate, filtered, and the filtrate was concentrated under reduced pressure to give 2-(4-(((tertbutyldiphenylsilyl)oxy)methyl)bicyclo[2.2.1]heptan-1-yl)acetaldehyde 7′ (2.2 g, 95% yield) as a crude residue, which was used in the next step without further purification.

Step 6′ 2-(4-(((tert-butyldiphenylsilyl)oxy)methyl)bicyclo[2.2.1]heptan-1-yl)ethan-1-ol 8′: To a stirred suspension of 2-(4-(((tertbutyldiphenylsilyl)oxy)methyl)bicyclo[2.2.1]heptan-1-yl)acetaldehyde 7′ (2.2 g, 5.41 mmol, 1.0 eq.) in MeOH (25 mL) was added sodium borohydride (0.409 g, 10.82 mmol, 2.0 eq.) under nitrogen atmosphere at 0° C. The resulting mixture was stirred at room temperature for 2 h. The reaction mixture was quenched with ice-cold water (150 mL) and extracted with ethyl acetate (2×150 mL). The combined organic phases were washed with brine (100 mL), dried over sodium sulfate, and filtered, and the filtrate was concentrated under reduced pressure to give the crude product. The crude product was purified by column chromatography using silica gel (230-400 mesh) with 20% ethyl acetate/pet ether to afford 2-(4-(((tert-butyldiphenylsilyl)oxy)methyl)bicyclo[2.2.1]heptan-1-yl)ethan-1-ol 8′ (1.2 g, 54.3% yield) as a colorless liquid.

¹H NMR (400 MHz, CDCl₃): δ ppm 1.07 (s, 9H), 1.22 (s, 2H), 1.28-1.66 (m, 8H), 1.82 (t, J=7.6 Hz, 2H), 3.69 (s, 2H), 3.73 (t, J=7.2 Hz, 2H), 7.37-7.44 (m, 6H), 7.66-7.69 (m, 4H).

Step 7′ tert-butyldiphenyl((4-(2-((tetrahydro-2H-pyran-2-yl)oxy)ethyl)bicyclo[2.2.1]heptan-1-yl)methoxy)silane 9′: To a stirred suspension of 2-(4-(((tertbutyldiphenylsilyl)oxy)methyl)bicyclo[2.2.1]heptan-1-yl)ethan-1-ol 8′ (1.2 g, 2.94 mmol, 1.0 eq.) in DCM (20 mL) was added DHP (0.322 mL, 3.52 mmol, 1.2 eq) and p-TsOH (0.559 g, 2.94 mmol, 1.0 eq) at 0° C. and the resulting mixture was stirred at room temperature for 1 h. Water (100 mL) was added to the reaction mixture and extracted with ethyl acetate (2×25 mL). The combined organic phases were washed with saturated sodium bicarbonate solution (100 mL), dried over sodium sulfate, and filtered, and the filtrate was concentrated under reduced pressure to afford the crude product. The crude product was purified by column chromatography using silica gel (230-400 mesh) with 5% ethyl acetate/pet ether to afford tert-butyldiphenyl((4-(2-((tetrahydro-2H-pyran-2-yl)oxy)ethyl)bicyclo[2.2.1]heptan-1-yl)methoxy)silane 9′ (1.3 g, 90% yield) as a colorless liquid.

¹H NMR (400 MHz, CDCl₃): δ ppm 0.88-0.93 (m, 2H), 1.07 (s, 9H), 1.22-1.73 (m, 14H), 1.84 (t, J=7.2 Hz, 2H), 3.43-3.56 (m, 2H), 3.69 (s, 2H), 3.81-3.93 (m, 2H), 4.60-4.62 (m, 1H), 7.37-7.44 (m, 6H), 7.66-7.69 (m, 4H).

Step 8′ (4-(2-((tetrahydro-2H-pyran-2-yl)oxy)ethyl)bicyclo[2.2.1]heptan-1-yl)methanol 10′: To a stirred suspension of tert-butyldiphenyl((4-(2-((tetrahydro-2H-pyran-2-yl)oxy)ethyl)bicyclo[2.2.1]heptan-1-yl)methoxy)silane 9′ (1.3 g, 2.64 mmol, 1.0 eq.) in anhydrous THF (20 mL) was added 1 M TBAF in THF (6.60 mL, 6.60 mmol, 2.5 eq) under nitrogen atmosphere at 0° C. and the resulting mixture was stirred at room temperature for 2 h. The reaction mixture was quenched with saturated ammonium chloride solution (70 mL) and extracted with EtOAc (2×150 mL). Organic phases were combined and washed with brine (100 mL). Combined organic phases were dried over anhydrous Na₂SO₄, filtered, and the filtrate was concentrated under reduced pressure to give the crude product. The crude product was purified by column chromatography using silica gel (230-400 mesh) with 20-30% EtOAc/pet ether to afford (4-(2-((tetrahydro-2H-pyran-2-yl)oxy)ethyl)bicyclo[2.2.1]heptan-1-yl)methanol 10′ (450 mg, 1.769 mmol, 67.1% yield) as a colorless liquid.

¹H NMR (400 MHz, DMSO-d6): δ ppm 1.08 (s, 2H), 1.17-1.22 (m, 2H), 1.31-1.34 (m, 2H), 1.40-1.70 (m, 10H), 1.71 (t, J=7.2 Hz, 2H), 3.34-3.44 (m, 4H), 3.66-3.73 (m, 2H), 4.37 (t, J=4.8 Hz, 1H), 4.54 (d, J=4.0 Hz, 1H).

Step 9′. 4-(2-((tetrahydro-2H-pyran-2-yl)oxy)ethyl)bicyclo[2.2.1]heptane-1-carbaldehyde 11′: To a stirred solution of (4-(2-((tetrahydro-2H-pyran-2-yl)oxy)ethyl)bicyclo[2.2.1]heptan-1-yl)methanol 10′ (50 mg, 0.197 mmol, 1.0 eq.) in anhydrous DMSO (1 mL) was added IBX (110 mg, 0.393 mmol, 2.0 eq.) at room temperature. The resulting mixture was stirred at room temperature for 2 h. Water was added to the reaction mixture and extracted with ethyl acetate (2×25 mL). The combined organic phases were washed with saturated sodium bicarbonate solution (50 mL), dried over sodium sulfate, filtered, and the filtrate was concentrated under reduced pressure to afford 4-(2-((tetrahydro-2H-pyran-2-yl)oxy)ethyl)bicyclo[2.2.1]heptane-1-carbaldehyde 11′ (50 mg), which was used in the next step without further purification.

¹H NMR (400 MHz, CDCl₃): δ ppm 1.50-2.04 (m, 18H), 3.43-3.55 (m, 2H), 3.82-3.89 (m, 2H), 4.59 (t, J=2.8 Hz, 1H), 9.82 (s, 1H)

Step 10′ 2-(2-(4-ethynylbicyclo[2.2.1]heptan-1-yl)ethoxy)tetrahydro-2H-pyran 12′: To a stirred solution of 4-(2-((tetrahydro-2H-pyran-2-yl)oxy)ethyl)bicyclo[2.2.1]heptane-1-carbaldehyde 11′ (50 mg, 0.198 mmol, 1.0 eq.) in MeOH (2 mL) was added potassium carbonate (54.8 mg, 0.396 mmol, 2.0 eq.) and Bestmann-Ohira reagent (10% in acetonitrile) (0.285 mg, 0.238 mmol, 1.2 eq.) under nitrogen atmosphere at room temperature. The resulting mixture was stirred at room temperature for 1 h. Water (50 mL) was added to the reaction mixture and extracted with ethyl acetate (2×25 mL). The combined organic phases were washed with brine (25 mL), dried over sodium sulfate, and filtered, and the filtrate was concentrated under reduced pressure to give 2-(2-(4-ethynylbicyclo[2.2.1]heptan-1-yl)ethoxy)tetrahydro-2H-pyran 12′ (50 mg), which was used in the next step without further purification.

Step 11′. 1-(4-(((R)-1-cyanoethyl)amino)-5-(4-(4-(2-((tetrahydro-2H-pyran-2-yl)oxy)ethyl)bicyclo[2.2.1]heptan-1-yl)-1H-1,2,3-triazol-1-yl)pyridin-2-yl)-1H-pyrazolo[3,4-b]pyridine-5-carbonitrile 14′: To a stirred solution of (R)-1-(5-azido-4-((1-cyanoethyl)amino)pyridin-2-yl)-1H-pyrazolo[3,4-b]pyridine-5-carbonitrile A-57 (275 mg, mmol, 1.0 eq.) and 2-(2-(4-ethynylbicyclo[2.2.1]heptan-1-yl)ethoxy)tetrahydro-2H-pyran 12′ (248 mg, 0.999 mmol, 1.2 eq.) in acetone (8 mL) was added sodium ascorbate (82 mg, 0.416 mmol, 0.5 eq.) and copper(II) sulfate pentahydrate (41.6 mg, 0.167 mmol, 0.2 eq.) in water (2 mL) at room temperature. The resulting reaction mixture was stirred at room temperature for 2 h. Water (50 mL) was added into the reaction mixture and extracted with ethyl acetate (2×150 mL). The combined organic phases were washed with brine (100 mL), dried over sodium sulfate, filtered and the filtrate was concentrated under reduced pressure to give a crude product. The crude product was purified by column chromatography using silica gel (230-400 mesh) with 80%-100% ethyl acetate/pet ether to afford 1-(4-(((R)-1-cyanoethyl)amino)-5-(4-(4-(2-((tetrahydro-2H-pyran-2-yl)oxy)ethyl)bicyclo[2 0.2.1]heptan-1-yl)-1H-1,2,3-triazol-1-yl)pyridin-2-yl)-1H-pyrazolo[3,4-b]pyridine-5-carbonitrile 14′ (270 mg, 53.2% yield) as a brown solid.

LCMS method 1: retention time: 1.943 min, 95.9% purity at 220 nm, [M+H]⁺=579.0.

Step 12′ (R)-1-(4-((1-cyanoethyl)amino)-5-(4-(4-(2-hydroxyethyl)bicyclo[2.2.1]heptan-1-yl)-1H-1,2,3-triazol-1-yl)pyridin-2-yl)-1H-pyrazolo[3,4-b]pyridine-5-carbonitrile 15′: To a stirred solution of 1-(4-(((R)-1-cyanoethyl)amino)-5-(4-(4-(2-((tetrahydro-2H-pyran-2-yl)oxy)ethyl)bicyclo[2.2.1]heptan-1-yl)-1H-1,2,3-triazol-1-yl)pyridin-2-yl)-1H-pyrazolo[3,4-b]pyridine-5-carbonitrile 14′ (270 mg, mmol, 1.0 eq.) in anhydrous MeOH (6 mL) was added pTsOH (44.4 mg, 0.233 mmol, 0.5 eq.) under nitrogen atmosphere. The resulting mixture was stirred at room temperature for 1 h. Saturated sodium bicarbonate solution (25 mL) was added into the reaction mixture and extracted with DCM (2×50 mL). The combined organic phases were washed with brine (25 mL), dried over sodium sulfate, filtered and the filtrate was concentrated under reduced pressure to give (R)-1-(4-((l-cyanoethyl)amino)-5-(4-(4-(2-hydroxyethyl)bicyclo[2 0.2.1]heptan-1-yl)-1H-1,2,3-triazol-1-yl)pyridin-2-yl)-1H-pyrazolo[3,4-b]pyridine-5-carbonitrile 15′ (170 mg, 70.0% yield) as a brown solid, which was used in the next step without further purification.

LCMS method 1: retention time: 1.217 min, 95.2% purity at 220 nm, [M+H]⁺=494.8.

Step 13′. (R)-1-(4-((1-cyanoethyl)amino)-5-(4-(4-(2-oxoethyl)bicyclo[2.2.1]heptan-1-yl)-1H-1,2,3-triazol-1-yl)pyridin-2-yl)-1H-pyrazolo[3,4-b]pyridine-5-carbonitrile 16′: To a stirred solution of (R)-1-(4-((1-cyanoethyl)amino)-5-(4-(4-(2-hydroxyethyl)bicyclo[2.2.1]heptan-1-yl)-1H-1,2,3-triazol-1-yl)pyridin-2-yl)-1H-pyrazolo[3,4-b]pyridine-5-carbonitrile 15′ (100 mg, 0.202 mmol, 1.0 eq.) in anhydrous DMSO (3 mL) was added IBX (113 mg, 0.404 mmol, 2.0 eq.) under nitrogen atmosphere. The resulting mixture was stirred at room temperature for 2 h. The reaction was quenched with ice-cold water (25 mL) and extracted with ethyl acetate (2×50 mL). Organic phases were combined and washed with saturated sodium bicarbonate solution (25 mL). Combined organic phases were dried over anhydrous Na₂SO₄, filtered and concentrated under reduced pressure to afford (R)-1-(4-((1-cyanoethyl)amino)-5-(4-(4-(2-oxoethyl)bicyclo[2.2.1]heptan-1-yl)-1H-1,2,3-triazol-1-yl)pyridin-2-yl)-1H-pyrazolo[3,4-b]pyridine-5-carbonitrile 16′ (100 mg, 0.187 mmol, 92% yield) as an brown solid, which was used in the next step without further purification.

LCMS method 1: retention time: 1.379 min, 92.9% purity at 220 nm, [M+H]⁺=492.8

Step 14′ 1-(4-(((R)-1-cyanoethyl)amino)-5-(4-(4-(2-(4-(4-(2,6-dioxopiperidin-3-yl)phenyl)piperazin-1-yl)ethyl)bicyclo[2.2.1]heptan-1-yl)-1H-1,2,3-triazol-1-yl)pyridin-2-yl)-1H-pyrazolo[3,4-b]pyridine-5-carbonitrile, TFA P202: To a stirred solution of (R)-1-(4-((1-cyanoethyl)amino)-5-(4-(4-(2-oxoethyl)bicyclo[2.2.1]heptan-1-yl)-1H-1,2,3-triazol-1-yl)pyridin-2-yl)-1H-pyrazolo[3,4-b]pyridine-5-carbonitrile 16′ (100 mg, 0.203 mmol, 1.0 eq.) and 3-(4-(piperazin-1-yl)phenyl)piperidine-2,6-dione, HCl 17′ (82 mg, 0.264 mmol, 1.3 eq.) in anhydrous DMSO (3 mL) was added sodium triacetoxyborohydride (86 mg, 0.406 mmol, 2.0 eq.) under nitrogen atmosphere at room temperature. The resulting mixture was stirred at room temperature for 16 h. Water (50 mL) was added to the reaction mixture and extracted with DCM (2×100 mL). The combined organic phases were washed with brine (50 mL), dried over sodium sulfate, filtered and the filtrate was concentrated under reduced pressure to give crude product. Crude product was purified by preparative HPLC to afford 1-(4-(((R)-1-cyanoethyl)amino)-5-(4-(4-(2-(4-(4-(2,6-dioxopiperidin-3-yl)phenyl)piperazin-1-yl)ethyl)bicyclo[2.2.1]heptan-1-yl)-1H-1,2,3-triazol-1-yl)pyridin-2-yl)-1H-pyrazolo[3,4-b]pyridine-5-carbonitrile, TFA P202 (100 mg, 0.113 mmol, 55.6% yield) as an off-white solid. Prep-HPLC method: Phenyl C8, 250×19 mm, 5.0 Mobile phase: A: 0.1% TFA in MQ-water; B: Acetonitrile; Flow rate: 15 mL/min;

LCMS method 2: retention time: 1.362 min, 97.52% purity at 220 nm, [M+H]⁺=750.4.

¹H NMR (400 MHz, DMSO-d6): δ ppm 1.52-1.56 (m, 2H), 1.60-1.70 (m, 7H), 1.78-1.85 (m, 2H), 1.98-2.05 (m, 3H), 2.11-2.22 (m, 3H), 2.60-2.70 (m, 1H), 2.92-3.02 (m, 2H), 3.15-3.32 (m, 4H), 3.64-3.67 (m, 2H), 3.75-3.80 (m, 1H), 3.84-3.88 (m, 2H), 5.02-5.06 (m, 1H), 6.99 (d, J=8.8 Hz, 2H), 7.13 (d, J=8.8 Hz, 2H), 7.24 (d, J=8.4 Hz, 1H), 7.68 (s, 1H), 8.42 (s, 1H), 8.44 (s, 1H), 8.73 (s, 1H), 9.06-9.08 (m, 2H), 10.81 (s, 1H). Table 46 summarizes the compounds prepared using General Procedure X-17.

TABLE 46
Final Compounds Prepared via General Procedure X-17

| Compound | TMB | CBM | |
|---|---|---|---|
| No. | Portion | Portion | Structure |
| P-202 | A-57 | C-12 | 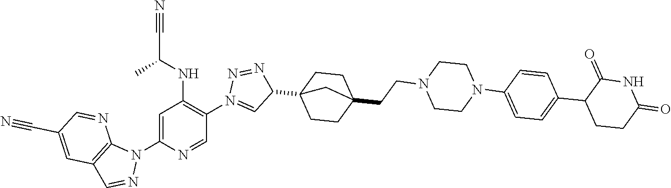 |
| P-212 | A-57 | C-12 | 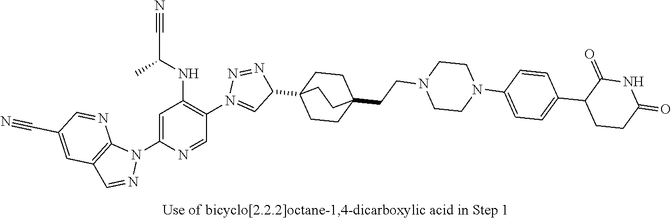 |

| Compound | |
|---|---|
| No. | Characterization |
| P-202 | 55.6% yield as an off-white solid. |
| | LCMS method 7: Retention time: 1.360 min, 97.2% purity at 215 |
| | nm, (M + H) = 750.4 |
| | ¹H NMR (400 MHz, DMSO-d6): δ ppm 1.52-1.56 (m, 2H), 1.60- |
| | 1.70 (m, 7H), 1.78-1.85 (m, 2H), 1.98-2.05 (m, 3H), 2.11- |
| | 2.22 (m, 3H), 2.60-2.70 (m, 1H), 2.92-3.02 (m, 2H), 3.15-3.32 |
| | (m, 4H), 3.64-3.67 (m, 2H), 3.75-3.80 (m, 1H), 3.84-3.88 (m, |
| | 2H), 5.02-5.06 (m, 1H), 6.99 (d, J = 8.8 Hz, 2H), 7.13 (d, J = 8.8 |
| | Hz, 2H), 7.24 (d, J = 8.4 Hz, 1H), 7.68 (s, 1H), 8.42 (s, 1H), 8.44 |
| | (s, 1H), 8.73 (s, 1H), 9.06-9.08 (m, 2H), 10.81 (s, 1H). |
| | One proton was not apparent by ¹H NMR. |
| P-212 | 28.3% yield as an off-white solid. |
| | LCMS method 7: Retention time: 1.769 min, 99.4% purity at 215 |
| | nm, [M + H]⁺ = 764.4 |
| | ¹H NMR (400 MHz, DMSO-d6): δ ppm 1.51-1.60 (m, 8H), 1.62 |
| | (d, J = 7.2 Hz, 3H), 1.90-1.94 (m, 6H), 1.98-2.03 (m, 1H), 2.14- |
| | 2.19 (m, 1H), 2.46-2.48 (m, 1H), 2.65-2.68 (m, 1H), 2.92-2.98 |
| | (m, 2H), 3.14-3.22 (m, 4H), 3.61-3.65 (m, 2H), 3.75-3.87 (m, |
| | 3H), 5.01-5.05 (m, 1H), 6.99 (d, J = 8.8 Hz, 2H), 7.13 (d, J = 8.8 |
| | Hz, 2H), 7.23-7.25 (m, 1H), 7.68 (m, 1H), 8.37 (s, 1H), 8.43 (s, |
| | 1H), 8.73 (s, 1H), 9.07-9.09 (s, 2H), 10.82 (s, 1H). |

General Procedure X-18. The scheme shown below for the synthesis of P-226 is provided as a representative synthesis for General Procedure X-18.

### Example S58. 1-(4-(((R)-1-cyanoethyl)amino)-5-(4-(4-(2-(4-(4-(2,6-dioxopiperidin-3-yl)phenyl)piperazin-1-yl)ethyl)-4-hydroxycyclohexyl)-1H-1,2,3-triazol-1-yl)pyridin-2-yl)-1H-pyrazolo[3,4-b]pyridine-5-carbonitrile (P-226)

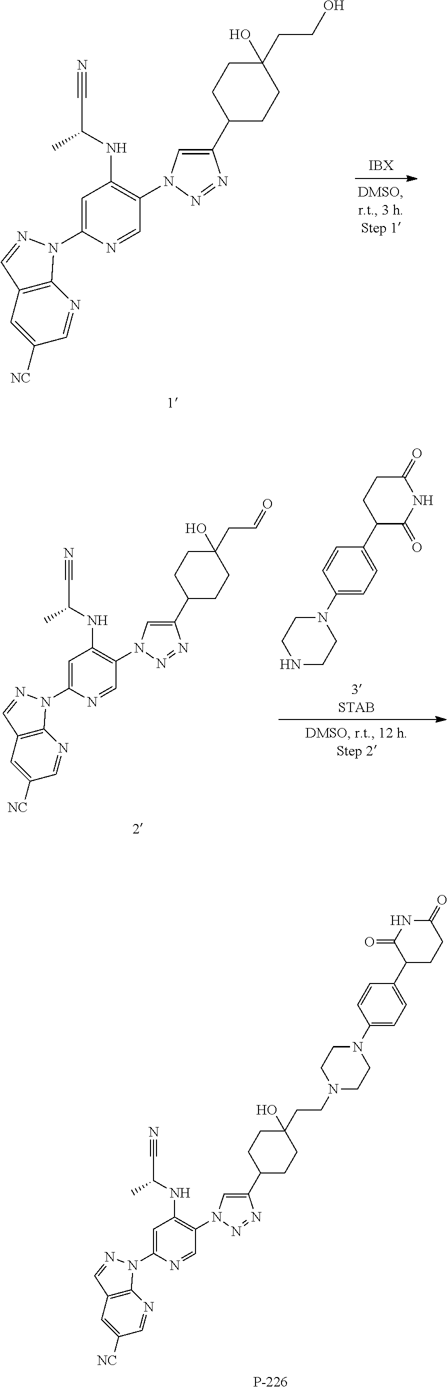 [SMILES: C[C@H](C#N)Nc1cc(-n2ncc3cc(C#N)cnc32)ncc1-n1cc(C2CCC(O)(CCO)CC2)nn1 ; C[C@H](C#N)Nc1cc(-n2ncc3cc(C#N)cnc32)ncc1-n1cc(C2CCC(O)(CC=O)CC2)nn1 ; O=C1CCC(c2ccc(N3CCNCC3)cc2)C(=O)N1 ; C[C@H](C#N)Nc1cc(-n2ncc3cc(C#N)cnc32)ncc1-n1cc(C2CCC(O)(CCN3CCN(c4ccc(C5CCC(=O)NC5=O)cc4)CC3)CC2)nn1]

LCMS Method 1. Column: Kinetex XB-C18, 50×4.6 mm, 5 μm, Temperature: RT, Flow: 1.0 mL/min, Run Time: 6 min, Mobile Phase Conditions: Mobile phase-A: 0.1% TFA in H₂O, Mobile phase-B: 0.1% TFA in CH₃CN, Gradient: Initial 95% Mobile Phase A and 5% Mobile Phase B linear gradient to 95% Mobile Phase B for 2.5 min. MSD positive.

Step 1′. Preparation of 1-(4-(((R)-1-cyanoethyl)amino)-5-(4-(4-hydroxy-4-(2-oxoethyl)cyclohexyl)-1H-1,2,3-triazol-1-yl)pyridin-2-yl)-1H-pyrazolo[3,4-b]pyridine-5-carbonitrile 2′: To a solution of 1-(4-(((R)-1-cyanoethyl)amino)-5-(4-((1s,4S)-4-hydroxy-4-(2-hydroxyethyl)cyclohexyl)-1H-1,2,3-triazol-1-yl)pyridin-2-yl)-1H-pyrazolo[3,4-b]pyridine-5-carbonitrile 1′ (130 mg, 0.261 mmol, 1.0 eq.) in DMSO (2.0 mL, 0.130 M) under nitrogen atmosphere was added IBX (219 mg, 0.782 mmol, 3.0 eq.). The reaction was stirred at RT for 2 h, treated with cold-water (10.0 mL) and the aqueous phase was extracted with EtOAc (3×20 mL). The combined organic phases were washed with sodium bicarbonate solution (10.0 mL), then brine (20.0 mL), dried over sodium sulphate, filtered and concentrated under reduced pressure to obtain the crude product 1-(4-(((R)-1-cyanoethyl)amino)-5-(4-(4-hydroxy-4-(2-oxoethyl)cyclohexyl)-1H-1,2,3-triazol-1-yl)pyridin-2-yl)-1H-pyrazolo[3,4-b]pyridine-5-carbonitrile 2′ (390 mg), which was used in the next step without further purification.

LCMS method 1: retention time: 2.108 min, [M+H]⁺=497.3.

Step 2′. Preparation of 1-(4-(((R)-1-cyanoethyl)amino)-5-(4-(4-(2-(4-(4-(2,6-dioxopiperidin-3-yl)phenyl)piperazin-1-yl)ethyl)-4-hydroxycyclohexyl)-1H-1,2,3-triazol-1-yl)pyridin-2-yl)-1H-pyrazolo[3,4-b]pyridine-5-carbonitrile P-226: To a solution of 1-(4-(((R)-1-cyanoethyl)amino)-5-(4-((1s,4S)-4-hydroxy-4-(2-oxoethyl)cyclohexyl)-1H-1,2,3-triazol-1-yl)pyridin-2-yl)-1H-pyrazolo[3,4-b]pyridine-5-carbonitrile 2′ (130 mg, 0.262 mmol, 1.0 eq.) in DMSO (2.0 mL, 0.130 M) under nitrogen atmosphere was added 3-(4-(piperazin-1-yl)phenyl)piperidine-2,6-dione 2′ (79 mg, 0.288 mmol, 1.1 eq) and stirred the reaction mass for min at RT. STAB (166 mg, 0.785 mmol, 3.0 eq.) was added to the reaction mixture and stirred for another 3 h at same temperature. The reaction mixture was quenched with water (10 drops) and purified by preparative HPLC purification to give 1-(4-(((R)-1-cyanoethyl)amino)-5-(4-(4-(2-(4-(4-(2,6-dioxopiperidin-3-yl)phenyl)piperazin-1-yl)ethyl)-4-hydroxycyclohexyl)-1H-1,2,3-triazol-1-yl)pyridin-2-yl)-1H-pyrazolo[3,4-b]pyridine-5-carbonitrile P-226 (24.0 mg, 9.06% yield). Prep-HPLC method: X-select C18, 250×19 mm, 5 Mobile phase A: 0.1% TFA in water, Mobile phase B: acetonitrile, Flow rate: 15.0 mL/min.

LCMS method 1: retention time: 2.012 min, 97.36% purity at 220 nm, [M+H]⁺=754.3.

¹H NMR (400 MHz, DMSO-d6): δ ppm 1.40-1.55 (m, 2H), 1.62 (d, J=7.2 Hz, 3H), 1.65-1.90 (m, 7H), 1.91-2.02 (m, 3H), 2.65-2.80 (m, 2H), 2.90-3.05 (m, 2H), 3.13-3.38 (m, 4H), 3.74-3.91 (m, 3H), 4.51 (brs, 1H), 4.99-5.10 (m, 1H), 6.96-7.00 (m, 2H), 7.09-7.14 (m, 2H), 7.20-7.22 (m, 1H), 7.67-7.69 (m, 1H), 8.36-8.43 (m, 2H), 8.73 (s, 1H), 9.06-9.08 (m, 2H), 10.80 (s, 1H).

Table 47 summarizes the compounds prepared using General Procedure X-18.

TABLE 47
Final Compounds Prepared via General Procedure X-18

| Compound | TMB | CBM | |
|---|---|---|---|
| No. | Portion | Portion | Structure |
| P-226 | A-85 | C-12 | 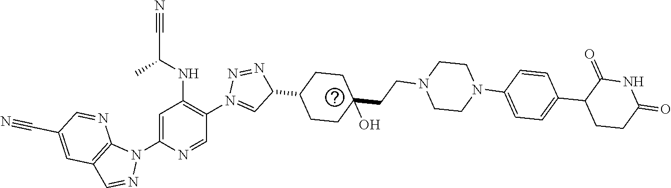 |
| P-227 | A-85i | C-12 | 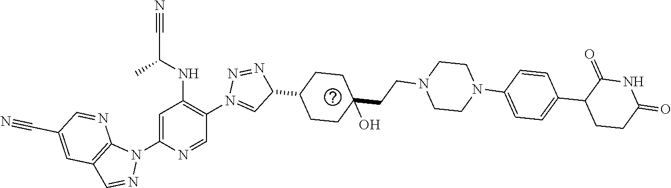 |

| Compound | |
|---|---|
| No. | Characterization |
| P-226 | 9.1% yield as an off-white solid. |
| | LCMS method 8: Retention time: 2.012 min, 97.4% purity at 215 |
| | nm, [M + H]⁺ = 754.3 |
| | ¹H NMR (400 MHz, DMSO-d6): δ ppm 1.40-1.55 (m, 2H), 1.62 |
| | (d, J = 7.2 Hz, 3H), 1.65-1.90 (m, 7H), 1.91-2.02 (m, 3H), 2.65- |
| | 2.80 (m, 2H), 2.90-3.05 (m 2H), 3.13-3.38 (m, 4H), 3.74-3.91 |
| | (m, 3H), 4.51 (brs, 1H), 4.99-5.10 (m, 1H), 6.96-7.00 (m, 2H), |
| | 7.09-7.14 (m, 2H), 7.20-7.22 (m, 1H), 7.67-7.69 (m, 1H), 8.36- |
| | 8.43 (m, 2H), 8.73 (s, 1H), 9.06-9.08 (m, 2H), 10.80 (s, 1H) |
| | Three protons were not apparent by ¹H NMR. |
| P-227 | 30% yield as an off-white solid. |
| | LCMS method 8: Retention time: 2.006 min, 96.4% purity at 215 |
| | nm, [M + H]⁺ = 754.4 |
| | ¹H NMR (400 MHz, DMSO-d6): δ ppm 1.45-1.58 (m, 2H), 1.60- |
| | 1.63 (m, 3H), 1.68-1.92 (m, 7H), 1.98-2.21 (m, 4H), 2.75-2.97 |
| | (m, 3H), 3.12-3.22 (m, 2H), 3.64-3.66 (m, 2H), 3.75-3.86 (m, |
| | 3H), 4.48 and 4.70 (s, 1H), 4.99-5.08 (m, 1H), 6.96-7.00 (m, |
| | 2H), 7.11-7.14 (m, 2H), 7.19-7.22 (m, 1H), 7.68 (d, J = 2.00 Hz, |
| | 1H), 8.36-8.42 (m, 2H), 8.73 (s, 1H), 9.06-9.08 (m, 2H), 10.79- |
| | 10.80 (m, 1H) |
| | Three protons were not apparent by ¹H NMR. |

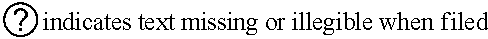

Table 48 summarizes the intermediates prepared using the general procedure of TBM-4.

TABLE 48
Intermediate Compounds Prepared via General Procedure TBM-4

| Inter- | |
|---|---|
| mediate | |
| No. | Structure |
| A-89 | 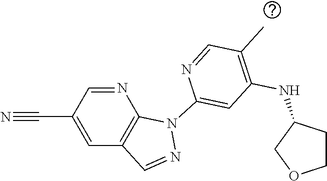 [SMILES: Cc1cnc(-n2ncc3cc(C#N)cnc32)cc1N[C@@H]1CCOC1] |
| A-90 | 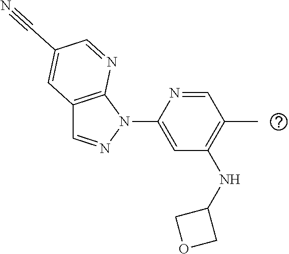 [SMILES: Cc1cnc(-n2ncc3cc(C#N)cnc32)cc1NC1COC1] |
| A-91 | 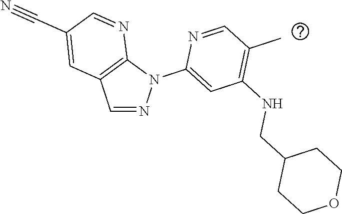 [SMILES: Cc1cnc(-n2ncc3cc(C#N)cnc32)cc1NCC1CCOCC1] |
| A-92 | 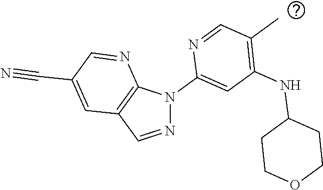 [SMILES: Cc1cnc(-n2ncc3cc(C#N)cnc32)cc1NC1CCOCC1] |
| A-93 | 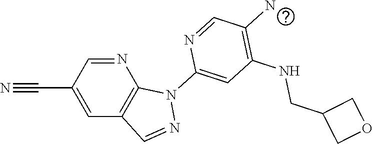 [SMILES: N#Cc1cnc2c(cnn2-c2cc(NCC3COC3)c(N)cn2)c1] |
| A-94 | 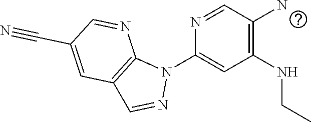 [SMILES: CCNc1cc(-n2ncc3cc(C#N)cnc32)ncc1N] |
| A-95 | 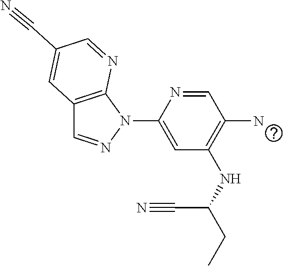 [SMILES: CC[C@H](C#N)Nc1cc(-n2ncc3cc(C#N)cnc32)ncc1N] |
| A-96 | 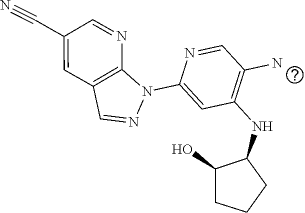 [SMILES: N#Cc1cnc2c(cnn2-c2cc(N[C@H]3CCC[C@H]3O)c(N)cn2)c1] |
| A-97 | 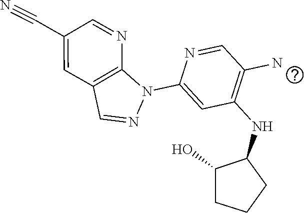 [SMILES: N#Cc1cnc2c(cnn2-c2cc(N[C@H]3CCC[C@@H]3O)c(N)cn2)c1] |
| A-98 | 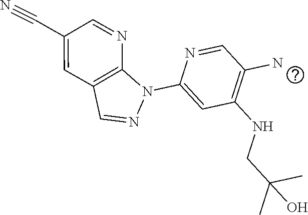 [SMILES: CC(C)(O)CNc1cc(-n2ncc3cc(C#N)cnc32)ncc1N] |
| A-99 | 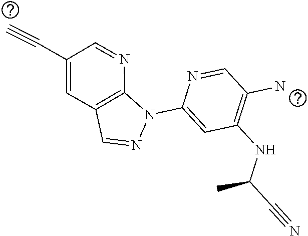 [SMILES: C#Cc1cnc2c(cnn2-c2cc(N[C@H](C)C#N)c(N)cn2)c1] |
| A-100 | 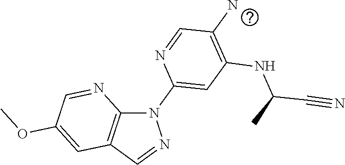 [SMILES: COc1cnc2c(cnn2-c2cc(N[C@H](C)C#N)c(N)cn2)c1] |

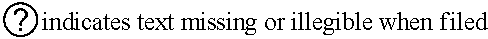

### Example S59. Preparation of 1-(5-azido-4-(cyclopentyloxy)pyridin-2-yl)-1H-pyrazolo[3,4-b]pyridine-5-carbonitrile (A-101)

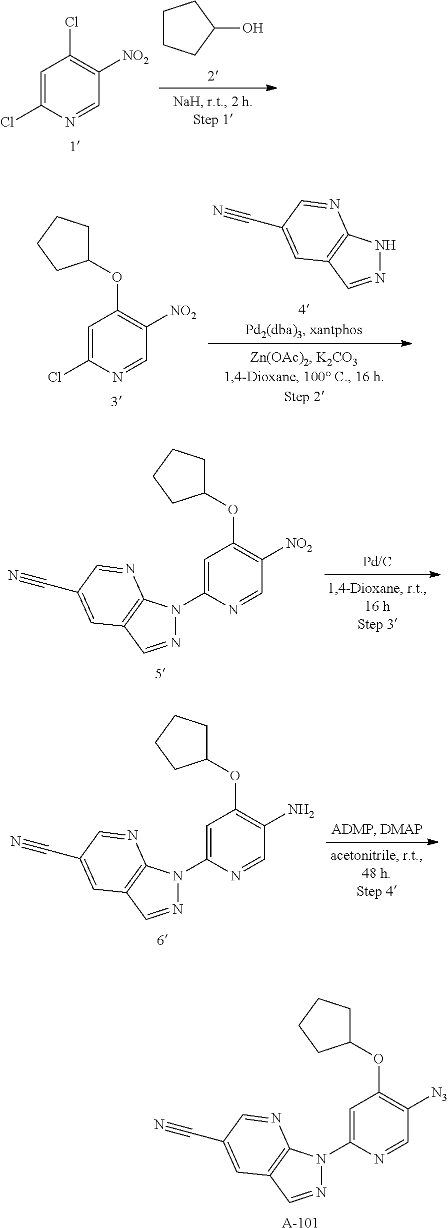

LCMS Method 1. Kinetex XB-C18, 50×4.6 mm, 5.0 μm. Temperature: RT, Flow: 1.0 mL/min, run time: 5.5 min. Mobile Phase Conditions: Mobile Phase-A: 0.1% TFA in H₂O, Mobile Phase-B: 0.1% TFA in ACN, Gradient: Initial 95% Mobile Phase A and 5% Mobile Phase B linear gradient to 95% Mobile Phase B for 2.5 min. MSD positive.

UPLC Method 2. Aquity BEH C18 (50×3.0)mm, 1.7 μm, Temperature: RT, Flow: 0.7 mL/min, run time: 3.0 min. Mobile Phase Conditions: Mobile Phase-A: 5.0 mM Ammonium formate:CH₃CN (98:02), Mobile Phase-B: CH₃CN: 5.0 mM Ammonium formate (98:02), Gradient: Initial 80% Mobile Phase A and 20% Mobile Phase B linear gradient to 98% Mobile Phase B for 1.5 min then hold for 2.0 minute. MSD positive.

Step 1′. Preparation of 2-chloro-4-(cyclopentyloxy)-5-nitropyridine 3′: To a cooled and stirred solution of the cyclopentanol 2′ (1.18 mL, 13.0 mmol, 1.0 eq.) in THF (20 mL) was added sodium hydride (0.311 g, 13 mmol, 1.0 eq.) followed by 2,4-dichloro-5-nitropyridine 1′ (2.50 g, 13.0 mmol, 1.0 eq.) stirred the reaction mixture at RT for 2 h. The reaction was quenched with ice, then diluted with ice-cold water (50 mL), and extracted with EA (2×100 mL). The combined organic layers were washed brine (50 mL), dried over anhydrous sodium sulphate, and concentrated under reduced pressure to get crude reaction mixture. The crude product was purified by column chromatography using silica gel (230-400 mesh) with 10% ethyl acetate in pet ether to get 2-chloro-4-(cyclopentyloxy)-5-nitropyridine 3′ (1.95 g, 7.88 mmol, 60.8% yield) as a white solid. 1-H-NMR (400 MHz, DMSO-d6): δ ppm 1.64-1.81 (m, 6H), 1.94-2.01 (m, 2H), 5.24-5.27 (m, 1H), 7.60 (s, 1H), 8.86 (s, 1H).

Step 2′. Preparation of 1-(4-(cyclopentyloxy)-5-nitropyridin-2-yl)-1H-pyrazolo[3,4-b]pyridine-5-carbonitrile 5′: To a stirred solution of 2-chloro-4-(cyclopentyloxy)-5-nitropyridine 3′ (1.5 g, 6.18 mmol, 1.0 eq.) and 1H-pyrazolo[3,4-b]pyridine-4′ (1.07 g, 7.42 mmol, 1.2 eq.) in 1,4-dioxane (50 mL) was added zinc acetate (0.045 g, 0.247 mmol, 0.04 eq.), K₂CO₃ (2.14 g, 15.5 mmol, 2.5 eq.) under N₂ atmosphere and then purged with nitrogen for 10 min. Xantphos (0.286 g, 0.495 mmol, 0.08) and Pd₂(dba)₃ (0.340 g, 0.371 mmol, 0.06 eq.) were added under N₂ atmosphere and the purging was continued for another 5 min. Then, the reaction mixture was stirred at 100° C. for 16 h. The reaction mixture was cooled to RT, filtered through a celite bed, and washed with ethyl acetate (2×30 mL). The filtrate was concentrated under reduced pressure to get the crude product. The crude product was purified by column chromatography using silica gel (230-400 mesh) with 50% ethyl acetate in pet ether to 1-(4-(cyclopentyloxy)-5-nitropyridin-2-yl)-1H-pyrazolo[3,4-b]pyridine-5-carbonitrile 5′ (1.50 g, 4.23 mmol, 68.4% yield) as a yellow solid. LCMS Method 1. retention time: 2.72 min, [M+H]⁺=351.1. ¹H-NMR (400 MHz, DMSO-d6): δ ppm 1.64-1.77 (m, 4H), 1.87-1.92 (m, 2H), 2.01-2.09 (m, 2H), 5.25-5.29 (m, 1H), 8.14 (s, 1H), 8.77 (s, 1H), 9.06 (d, J=2.0 Hz, 1H), 9.09 (s, 1H), 9.14 (d, J=2.0 Hz, 1H).

Step 3′. Preparation of 1-(5-amino-4-(cyclopentyloxy)pyridin-2-yl)-1H-pyrazolo[3,4-b]pyridine-5-carbonitrile 6′: To a stirred solution 1-(4-(cyclopentyloxy)-5-nitropyridin-2-yl)-1H-pyrazolo[3,4-b]pyridine-5-carbonitrile 5′ (3.0 g, 8.56 mmol, 1.0 eq.) in 1,4-dioxane (4.0 mL) was added 10% Pd—C(0.91 g) and stirred for 16 h at 25-30° C. The reaction mixture was filtered through celite bed, washed with 1,4-ioxane (2×10 mL), and concentrated under reduced pressure to get crude product 1-(5-amino-4-(cyclopentyloxy)pyridin-2-yl)-1H-pyrazolo[3,4-b]pyridine-5-carbonitrile 6′ (0.6 g, 1.41 mmol, 16.4% yield) as a pale yellow solid and it was used for the next step without further purification. LCMS Method 1: retention time: 2.17 min, [M+H]⁺=321.2.

Step 4′. Preparation of 1-(5-azido-4-(cyclopentyloxy)pyridin-2-yl)-1Hpyrazolo[3,4-b]pyridine-5-carbonitrile 7 (A-101): To a stirred solution of 1-(5-amino-4-(cyclopentyloxy)pyridin-2-yl)-1H-pyrazolo[3,4-b]pyridine-5-carbonitrile 6′ (0.6 g, 1.87 mmol, 1.0 eq.) in acetonitrile (6.0 mL) and DMF (0.667 mL) was added ADMP (1.07 g, 3.75 mmol, 2.0 eq.) and 4-dimethylaminopyridine (0.343 g, 2.81 mmol, 1.5 eq.) at room temperature and stirred for 16 h under nitrogen atmosphere. The reaction mixture was treated with ice-cold water (100 mL), filtered the precipitated solid through a Buchner funnel, and washed with water (50 mL) to get 1-(5-azido-4-(cyclopentyloxy)pyridin-2-yl)-1Hpyrazolo[3,4-b]pyridine-5-carbonitrile (A-101) (0.8 g, 88% yield) as brown solid. It was used for the next step without further purification. UPLC method 2: retention time: 1.60 min, [M+H]⁺=347.

Table 49 summarizes the intermediates prepared using a procedure similar to Example S59.

TABLE 49
Intermediate Compounds Prepared via a Procedure Similar
to Example S59.

| Inter- | |
|---|---|
| me- | |
| diate | |
| No. | Structure |
| A-102 | 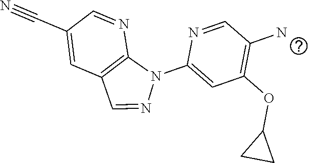 [SMILES: N#Cc1cnc2c(cnn2-c2cc(OC3CC3)c(N)cn2)c1] |
| A-103 | 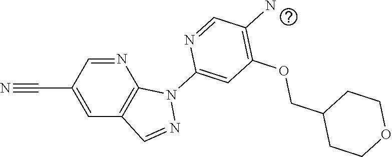 [SMILES: N#Cc1cnc2c(cnn2-c2cc(OCC3CCOCC3)c(N)cn2)c1] |

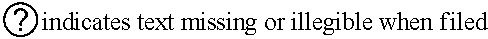

### Example S60. Preparation of (R)-6-((5-azido-4-((1-cyanoethyl)amino)pyridin-2-yl)amino)-(5-chloronicotinonitrile (A-104)

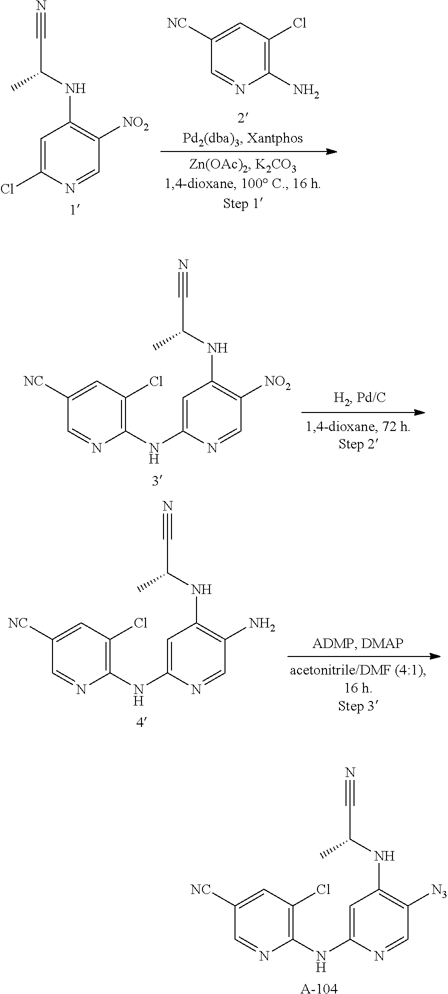

LCMS Method 1. Kinetex XB-C18, 50×4.6 mm, 5.0 Temperature: RT, Flow: 1.0 mL/min, run time: 5.5 min. Mobile Phase Conditions: Mobile Phase-A: 0.1% TFA in H₂O, Mobile Phase-B: 0.1% TFA in ACN, Gradient: Initial 95% Mobile Phase A and 5% Mobile Phase B linear gradient to 95% Mobile Phase B for 2.5 min. MSD positive.

LCMS Method 2. Kinetex XB-C18, 75×3.0 mm, 2.6 μm. Temperature: RT, Flow: 1.0 mL/min, run time: 5.0 min. Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 98% Mobile Phase A and 2% Mobile Phase B linear gradient to 100% Mobile Phase B for 4 min. MSD positive.

Step 1′. Preparation of (R)-5-chloro-6-((4-((1-cyanoethyl)amino)-5-nitropyridin-2-yl)amino)nicotinonitrile 3′: To a stirred solution of (R)-2-((2-chloro-5-nitropyridin-4-yl)amino)propanenitrile 1′ (2.5 g, 8.60 mmol) and 6-amino-5-chloronicotinonitrile 2′ (1.32 g, 8.60 mmol) in 1,4-dioxane (25 mL) was added Pd₂(dba)₃ (0.315 g, 0.344 mmol) and purged with nitrogen for 5 min. Xantphos (0.398 g, 0.688 mmol) followed by zinc acetate (0.474 g, 2.58 mmol) and K₂CO₃ (2.97 g, 21.5 mmol) were added to the reaction mixture and purged with nitrogen for 2 min. The reaction mixture was stirred at 100° C. for 16 h under nitrogen atmosphere. Then, the reaction mixture was filtered through a celite bed, and the celite bed was washed with 5% MeOH/DCM (500 mL). The filtrate was then transferred into a separating funnel containing water (400 mL) and extracted using 5% MeOH/DCM (2×250 mL). The combined organic extracts were washed with brine (100 mL), dried over sodium sulfate, filtered, and concentrated under reduced pressure. The obtained solid was washed with ethyl acetate (50 mL), filtered, and dried under vacuum to obtain (R)-5-chloro-6-((4-((1-cyanoethyl)amino)-5-nitropyridin-2-yl)amino)nicotinonitrile 3′ (2.13 g, 60.6% yield) as a brown solid. It was used for the next step without further purification. LCMS method 1: retention time: 2.59 min, [M+H]⁺=344.1. ¹H NMR (400 MHz, DMSO d6): δ ppm 1.74 (d, J=6.8 Hz, 3H), 4.96-5.04 (m, 1H), 7.78 (s, 1H), 8.34 (d, J=7.2 Hz, 1H), 8.54 (d, J=1.6 Hz, 1H), 8.71 (d, J=1.6 Hz, 1H), 9.00 (s, 1H), 9.58 (brs, 1H).

Step 2′. Preparation of (R)-6-((5-amino-4-((1-cyanoethyl)amino)pyridin-2-yl)amino)-5-chloronicotinonitrile 4′: To a stirred solution of (R)-5-chloro-6-((4-((1-cyanoethyl)amino)-5-nitropyridin-2-yl)amino)nicotinonitrile 3′ (2.10 g, 5.14 mmol) in 1,4-dioxane (20 mL) was added 10% palladium on carbon (0.547 g). The resulting reaction mixture was stirred under hydrogen atmosphere at room temperature for 72 h. The reaction mixture was filtered through a celite bed, and the celite bed was washed with 5% MeOH/DCM (150 mL). The filtrate was evaporated under reduced pressure to obtain (R)-6-((5-amino-4-((1-cyanoethyl)amino)pyridin-2-yl)amino)-5-chloronicotinonitrile 4′ (1.8 g, 76% yield) as a brown solid. It was used for the next step without further purification. LCMS method 1: retention time: 1.92 min, [M+H]⁺=314.1.

Step 3′. Preparation of (R)-6-((5-azido-4-((1-cyanoethyl)amino)pyridin-2-yl)amino)-5-chloronicotinonitrile (A-105): To a stirred solution of (R)-6-((5-amino-4-((1-cyanoethyl)amino)pyridin-2-yl)amino)-5-chloronicotinonitrile 4′ (500 mg, 1.08 mmol) and ADMP (649 mg, 2.16 mmol) in acetonitrile (10 mL) was added DMF (2.5 mL). Then, DMAP (264 mg, 2.161 mmol) was added to the reaction mixture, and the reaction mixture was stirred at room temperature for 16 h. Then, the reaction mixture was transferred into a conical flask containing ice, and the solution was stirred at room temperature for 30 min, which produced a brown solid. The solid was filtered, washed with ice-cold water, and evaporated to dryness to obtain (R)-6-((5-azido-4-((1-cyanoethyl)amino)pyridin-2-yl)amino)-5-chloronicotinonitrile (A-104) (513 mg, 0.907 mmol, 84% yield) as a brown solid. It was used for the next step without further purification. LCMS method 2: retention time: 1.742 min, [M+H]⁺=340.0.

Table 50 summarizes the intermediates prepared using a procedure similar to Example S60.

TABLE 50
Intermediate Compounds Prepared via a Procedure Similar
to Example S60

| Inter- | |
|---|---|
| mediate | |
| No. | Structure |
| A-105 | 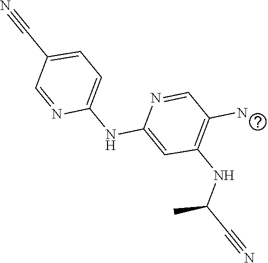 [SMILES: C[C@H](C#N)Nc1cc(Nc2ccc(C#N)cn2)ncc1N] |
| A-106 | 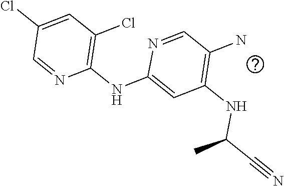 [SMILES: C[C@H](C#N)Nc1cc(Nc2ncc(Cl)cc2Cl)ncc1N] |
| A-107 | 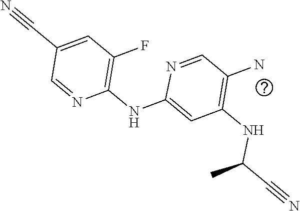 [SMILES: C[C@H](C#N)Nc1cc(Nc2ncc(C#N)cc2F)ncc1N] |
| A-108 | 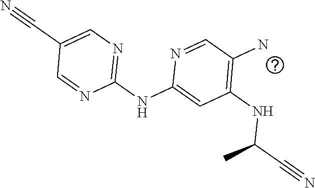 [SMILES: C[C@H](C#N)Nc1cc(Nc2ncc(C#N)cn2)ncc1N] |
| A-109 | 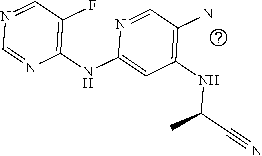 [SMILES: C[C@H](C#N)Nc1cc(Nc2ncncc2F)ncc1N] |
| A-110 | 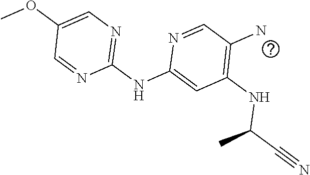 [SMILES: COc1cnc(Nc2cc(N[C@H](C)C#N)c(N)cn2)nc1] |
| A-111 | 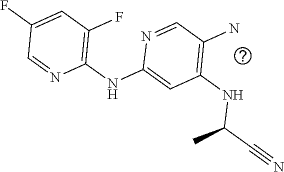 [SMILES: C[C@H](C#N)Nc1cc(Nc2ncc(F)cc2F)ncc1N] |

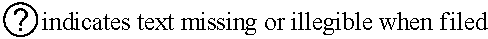

### Example S61: Preparation of 1-(5-azido-4-(((R)-1-cyanoethyl)amino)pyridin-2-yl)-6-((1R,4R)-2-oxa-5-azabicyclo[2.2.1]heptan-5-yl)-1H-pyrazolo[3,4-b]pyridine-5-carbonitrile (A-112)

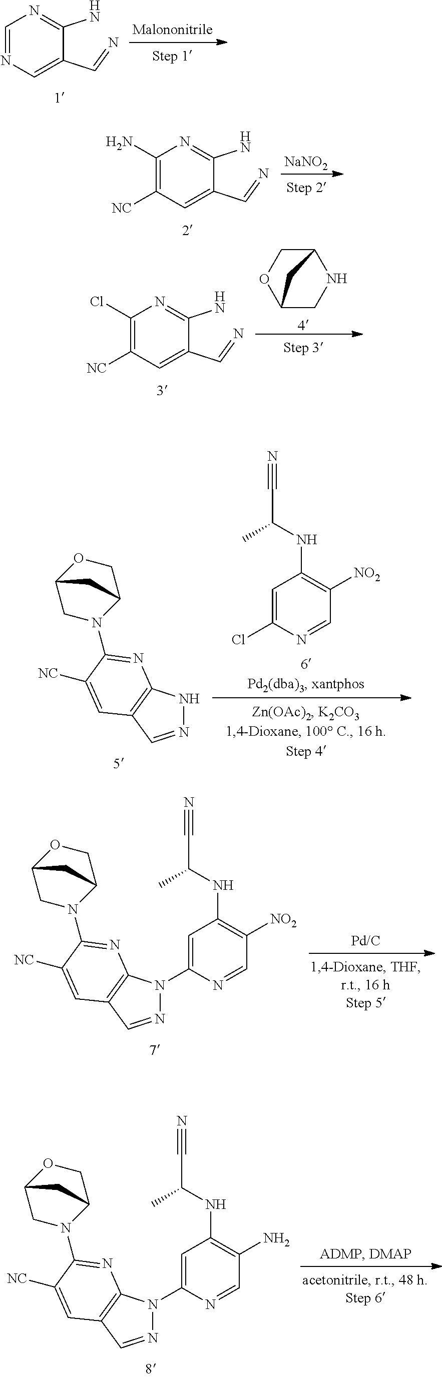

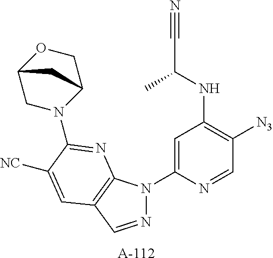

LCMS Method 1. Kinetex XB-C18, 75×3.0 mm, 2.6 Temperature: RT, Flow: 1.0 mL/min, run time: 5.0 min. Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 98% Mobile Phase A and 2% Mobile Phase B linear gradient to 100% Mobile Phase B for 4 min. MSD positive.

UPLC Method 2. Aquity BEH C18 (50×3.0 mm), 1.7 μm, Temperature: RT, Flow: 0.7 mL/min, run time: 2.6 min. Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 98% Mobile Phase B for 2 min. MSD positive.

Step 1′. Preparation of 6-amino-1H-pyrazolo[3,4-b]pyridine-5-carbonitrile 2′: To a stirred solution of the 1H-pyrazolo[3,4-d]pyrimidine 1′ (5 g, 41.6 mmol, 1.0 eq.) in 2-propanol (100 mL) for 45 min at 80° C. was added Malononitrile (21.2 mL, 333 mmol, 8.0 eq.) at the same temperature and stirred it for another 30 min. The reaction mixture was cooled to Rt and filtered to get a pale yellow solid, which was washed by 2-propanol (2×50 mL) to get pure 6-amino-1H-pyrazolo[3,4-b]pyridine-5-carbonitrile 2′ (4.5 g, 27.7 mmol, 66.6% yield) as a pale yellow solid. LCMS method 1: retention time: 1.67 min, 97.1% purity at 220 nm, [M+H]⁺=160.0. ¹H NMR (400 MHz, DMSO-d6): δ ppm 6.92 (brs, 2H), 7.89 (s, 1H), 8.34 (s, 1H), 13.12 (s, 1H).

Step 2′. Preparation of 6-chloro-1H-pyrazolo[3,4-b]pyridine-5-carbonitrile 3′: To an ice cooled and stirred solution of the 6-amino-1H-pyrazolo[3,4-b]pyridine-5-carbonitrile 2′ (4.5 g, 27.1 mmol, 1.0 eq.) in water (50 mL) was added conc. hydrochloric acid (50.0 mL) and then added sodium nitrite (3.75 g, 54.3 mmol, 2.0 eq.) slowly at cold temp. This resulting mixture was stirred at the same temperature for 45 min, and then the reaction mixture was stirred at 65° C. for 1 h. The reaction mixture was quenched with sodium bicarbonate solution (pH-7-8) and then extracted with ethyl acetate (3×50 mL). The combined organic layers were dried over sodium sulphate and concentrated under reduced pressure to afford 6-chloro-1H-pyrazolo[3,4-b]pyridine-5 carbonitrile 3′ (2.2 g, 12.2 mmol, 44.9% yield) pale yellow solid. It was used for the next step without further purification. LCMS method 1: retention time: 1.12 min, 98.8% purity at 220 nm, [M−H]=177.0. ¹H NMR (400 MHz, DMSO-d6): δ ppm 8.41 (s, 1H), 9.04 (s, 1H), 14.43 (s, 1H).

Step 3′. Preparation of 64(1R,4R)-2-oxa-5-azabicyclo[2.2.1]heptan-5-yl)-1H-pyrazolo[3,4-b]pyridine-5-carbonitrile 5′: To a stirred solution of the 6-chloro-1H-pyrazolo[3,4-b]pyridine-5-carbonitrile 3′ (1.0 g, 5.54 mmol, 1.0 eq.) and (1R,4R)-2-oxa-5-azabicyclo[2.2.1]heptane, HCl 4′ (1.128 g, 8.32 mmol, 1.5 eq.) in acetonitrile (10 mL) was added cesium carbonate (3.61 g, 11.1 mmol, 2.0 eq.) and heated the reaction mixture at 100° C. for 3 h. The reaction mixture was cooled to RT and filtered through a celite bed, and the residue was washed with ethyl acetate (2×60 mL). The filtrate was concentrated under reduced pressure to get the crude product. The crude product was purified by column chromatography using silica gel (230-400 mesh) with 15% ethyl acetate in pet ether to get 6-((1R,4R)-2-oxa-5-azabicyclo[2.2.1] heptan-5-yl)-1H-pyrazolo[3,4-b]pyridine-5-carbonitrile 5′ (1.2 g, 4.87 mmol, 88% yield) as pale-yellow solid. LCMS method 1: retention time: 1.063 min, 98.02% purity at 220 nm, [M+H]⁺=242.2. ¹H NMR (400 MHz, DMSO-d6): δ ppm 1.87 (d, J=10.0 Hz, 1H), 1.98 (dd, J=2.0 Hz and 10.0 Hz, 1H), 3.52 (d, J=10.00 Hz, 1H), 3.83-3.85 (m, 1H), 3.88-3.95 (m, 1H), 4.70 (s, 1H), 5.04 (s, 1H), 7.98 (s, 1H), 8.55 (s, 1H), 13.36 (s, 1H).

Step 4′. Preparation of 64(1R,4R)-2-oxa-5-azabicyclo[2.2.1] heptan-5-yl)-1-(4-4(R)-1-cyanoethyl)amino)-5-nitropyridin-2-yl)-1H-pyrazolo[3,4-b]pyridine-5-carbonitrile 7′: To a stirred solution of 6-((1R,4R)-2-oxa-5-azabicyclo[2.2.1]heptan-5-yl)-1Hpyrazolo[3,4-b]pyridine-5-carbonitrile 5′ (400 mg, 1.66 mmol, 1.0 eq.) and (R)-2-((2-chloro-5-nitropyridin-4-yl)amino)propanenitrile 6′ (564 mg, 2.49 mmol, 1.5 eq.) in 1,4-dioxane (10 mL) was added zinc acetate (122 mg, 0.663 mmol, 0.4 eq.), K₂CO₃ (573 mg, 4.15 mmol, 2.5 eq.) under N₂ atmosphere and purged with N₂ for 10 min. Then, Xantphos (48.0 mg, 0.083 mmol, 0.1 eq.) and Pd₂(dba)₃ (152 mg, 0.166 mmol, 0.05 eq.) were added under N₂ atmosphere, and the purging was continued for another 5 min. Then, the reaction mixture was stirred at 100° C. for 2 h. The reaction mixture was concentrated under reduced pressure to get crude RM, diluted with DCM (30 mL), stirred for 20 min, and filtered to get solid. The solid was washed by DCM (2×20 mL) and dried to get the crude product. The crude product was taken in water (50 mL), stirred for 30 min, and filtered the reaction mass through the Buchner to get a brown solid. It was then washed with water (2×20 mL) and dried to get 641R,4R)-2-oxa-5-azabicyclo[2.2.1]heptan-5-yl)-1-(4-(((R)-1-cyanoethyl)amino)-5-nitropyridin-2-yl)-1H pyrazolo[3,4-b]pyridine-5-carbonitrile 7′ (3.0 g) as a brown solid. The crude product was taken forward for the next step without further purification. UPLC method 2: retention time: 2.131 min, [M+H]⁺=432.0.

Step 5′. 1-(5-amino-4-4(R)-1-cyanoethyl)amino)pyridin-2-yl)-64(1R,4R)-2-oxa-5-azabicyclo[2.2.1] heptan-5-yl)-1H-pyrazolo[3,4-b]pyridine-5-carbonitrile 8′: To a solution of the 6-((1R,4R)-2-oxa-5-azabicyclo[2.2.1]heptan-5-yl)-1-(4-(((R)-1-cyanoethyl)amino)-5-nitropyridin-2-yl)-1H-pyrazolo[3,4-b]pyridine-5-carbonitrile 7′ (1.5 g) in 1,4-dioxane (15 mL) and THF (1.0 mL) was added 10% Pd—C(0.318 g, 2.99 mmol, 1.0 eq.). The reaction mixture was stirred at RT under hydrogen atmosphere for 16 h. The reaction mixture was filtered through a pad of celite bed, washed with dioxane (2×20 mL) and ethyl acetate (2×20 mL). Filtrate was concentrated under reduced pressure to get crude 1-(5-amino-44(R)-1-cyanoethyl)amino)pyridin-2-yl)-64(1R,4R)-2-oxa-5azabicyclo[2.2.1]heptan-5-yl)-1H-pyrazolo[3,4-b]pyridine-5-carbonitrile 8′ (0.6 g). It was used for the next step without further purification. UPLC method 2: retention time: 0.577 min, [M+H]⁺=402.2.

Step 6′. Preparation of 1-(5-azido-4-(((R)-1-cyanoethyl)amino)pyridin-2-yl)-6-41R,4R)-2-oxa-5-azabicyclo[2.2.1]heptan-5-yl)-1H-pyrazolo[3,4-b]pyridine-5-carbonitrile (A-112): To a stirred solution of 1-(5-amino-4-(((R)-1-cyanoethyl)amino)pyridin-2-yl)-6-((1R,4R)-2-oxa-5-azabicyclo[2.2.1]heptan-5-yl)-1H-pyrazolo[3,4-b]pyridine-5-carbonitrile 8′ (1.2 g, 2.81 mmol, 1.0 eq.) in acetonitrile (10 mL) and DMF (1.1 mL) was added ADMP (1.60 g, 5.62 mmol, 2.0 eq.) and 4-dimethylaminopyridine (0.515 g, 4.21 mmol, 1.5 eq.) at room temperature. The reaction mixture was stirred for 16 h at RT under nitrogen atmosphere. The reaction mixture was treated with ice-cold water (50 mL) and stirred for 1 h. The solid was filtered and washed with ice-cold water (2×10 mL) to get crude brown solid 1-(5-azido-44(R)-1-cyanoethyl)amino)pyridin-2-yl)-6-(1R,4R)-2-oxa-5-azabicyclo[2.2.1]heptan-5-yl)-1H-pyrazolo[3,4-b]pyridine-5-carbonitrile (A-112) (2.0 g). It was used for the next step without further purification. UPLC method 2: retention time: 1.078 min, [M+H]⁺=428.2.

Table 51 summarizes CBM intermediate compounds prepared via the General Procedure for CBM-4.

TABLE 51
CBM intermediates prepared via General Procedure for CMB-4

| Intermediate | Structure |
|---|---|
| No. | |
| C-83a peak 1 | 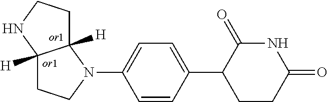 [SMILES: O=C1CCC(c2ccc(N3CC[C@@H]4NCC[C@@H]43)cc2)C(=O)N1] |
| C-83b peak 2 | 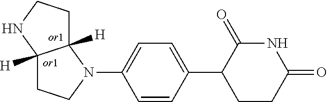 [SMILES: O=C1CCC(c2ccc(N3CC[C@@H]4NCC[C@@H]43)cc2)C(=O)N1] |
| C-84 | 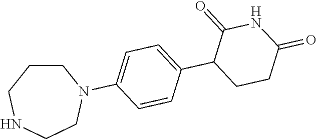 [SMILES: O=C1CCC(c2ccc(N3CCCNCC3)cc2)C(=O)N1] |
| C-85 | 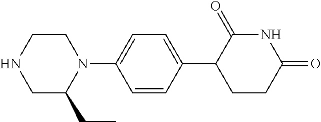 [SMILES: CC[C@H]1CNCCN1c1ccc(C2CCC(=O)NC2=O)cc1] |
| C-86 | 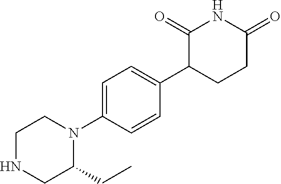 [SMILES: CC[C@@H]1CNCCN1c1ccc(C2CCC(=O)NC2=O)cc1] |

Table 52 summarizes CBM intermediates prepared via the General Procedure for CBM-11. All intermediates are enantiomerically pure.

TABLE 52
CBM intermediates prepared via General Procedure for CBM-11.

| Inter - | |
|---|---|
| mediate | |
| No. | Structure |
| C-87 | 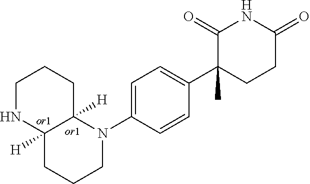 [SMILES: C[C@]1(c2ccc(N3CCC[C@H]4NCCC[C@H]43)cc2)CCC(=O)NC1=O] |
| C-88a peak 1 | 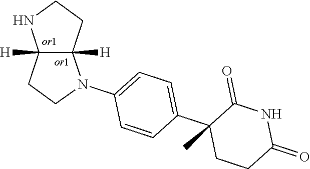 [SMILES: C[C@]1(c2ccc(N3CC[C@@H]4NCC[C@@H]43)cc2)CCC(=O)NC1=O] |
| C-88b peak 2 | 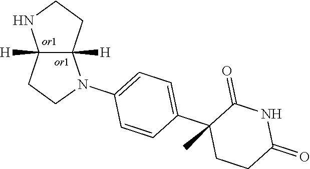 [SMILES: C[C@]1(c2ccc(N3CC[C@@H]4NCC[C@@H]43)cc2)CCC(=O)NC1=O] |
| C-89 | 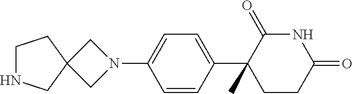 [SMILES: C[C@]1(c2ccc(N3CC4(CCNC4)C3)cc2)CCC(=O)NC1=O] |
| C-90 | 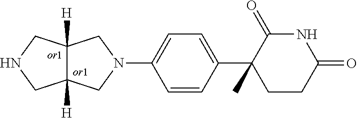 [SMILES: C[C@]1(c2ccc(N3C[C@H]4CNC[C@H]4C3)cc2)CCC(=O)NC1=O] |

### Example S62: Preparation of (R or S)-3-methyl-3-(5-(piperazin-1-yl)pyridin-2-yl)piperidine-2,6-dione (C-90a) and (S or R)-3-methyl-3-(5-(piperazin-1-yl)pyridin-2-yl)piperidine-2,6-dione (C-90b)

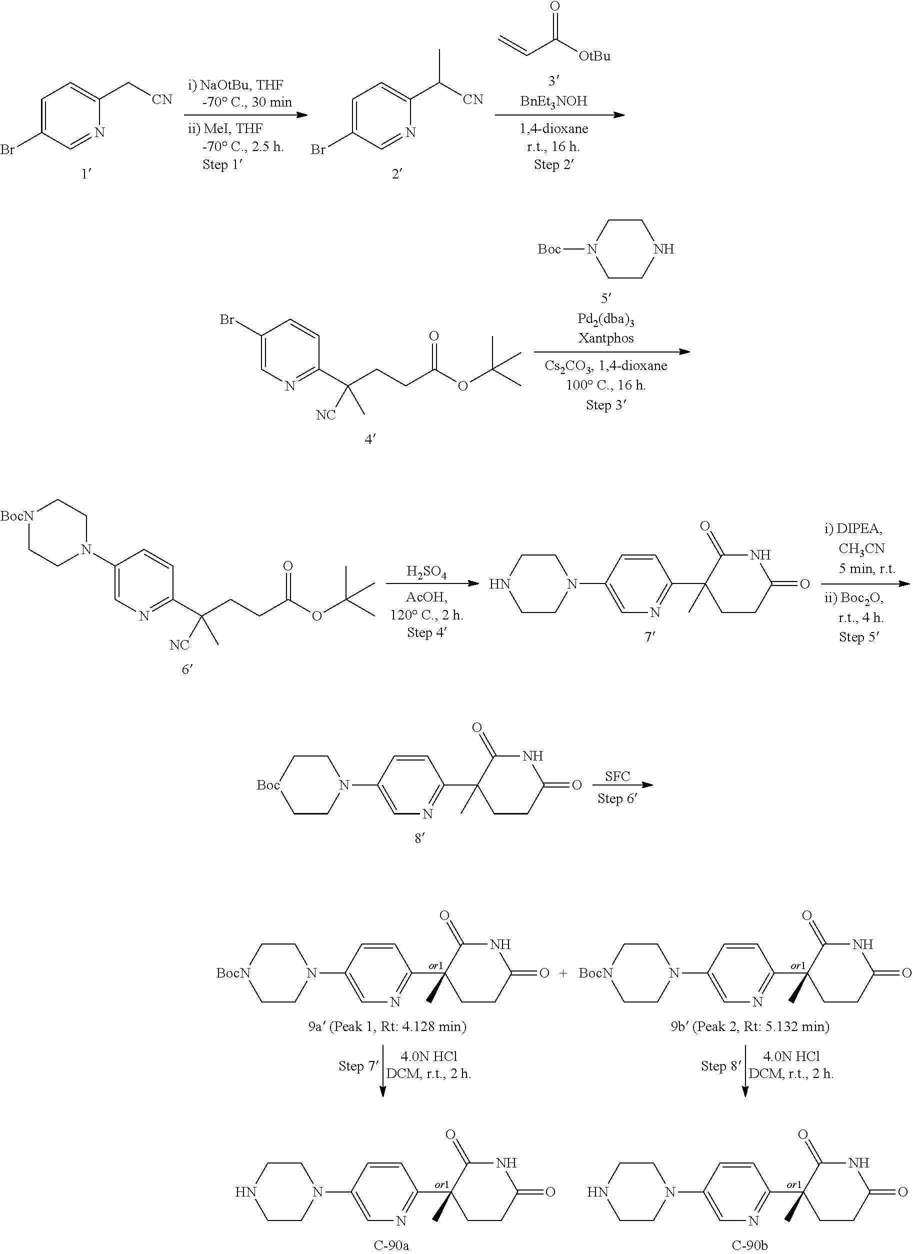

LCMS Method 1. Kinetex XB-C18, 75×3.0 mm, 2.6 Temperature: RT, Flow: 1.0 mL/min, run time: 5.0 min. Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 98% Mobile Phase A and 2% Mobile Phase B linear gradient to 100% Mobile Phase B for 4 min. MSD positive UPLC Method 2. Aquity BEH-C18, 50×2.1 mm, 1.7 Temperature: RT, Flow: 0.7 mL/min, run time: 2.5 min. Mobile Phase Conditions: Mobile Phase-A: 0.1% TFA in H₂O, Mobile Phase-B: 0.1% TFA in CAN, Gradient: Initial 80% Mobile Phase A and 20% Mobile Phase B linear gradient to 98% Mobile Phase B for 1.5 min. MSD positive.

Step 1′. Preparation of 2-(5-bromopyridin-2-yl)propanenitrile 2′: To a solution of 2-(5-bromopyridin-2-yl)acetonitrile 1″(5.0 g, 25.4 mmol) in THF (50 mL) was added sodium tert-butoxide (2.44 g, 25.4 mmol) portion wise over a period of 2 min at −70° C. and stirred under nitrogen for 30 min at the same temperature. Iodomethane (1.59 mL, 3.60 g, 25.4 mmol) was dissolved in THF (10 mL), and the resulting solution was added dropwise into the reaction mixture. The resulting reaction mixture was stirred at −70° C. under nitrogen for 2.5 h. The reaction mixture was warmed to room temperature, quenched with saturated ammonium chloride solution (100 mL), and extracted with ethyl acetate (150 mL). The organic layer was dried over sodium sulphate, filtered, and concentrated under reduced pressure to obtain the crude compound. The crude compound was purified by column chromatography using silica gel (230-400 mesh) with 6% ethyl acetate/pet ether to obtain 2-(5-bromopyridin-2-yl)propanenitrile 2′ (3.1 g, 56.7% yield) as a pale yellow liquid. LCMS method 1: retention time: 1.90 min, 98.4% purity at 220 nm, [M+H]⁺=211.2 and [M+2+H]⁺=213.0. ¹H NMR (400 MHz, CDCl₃): δ ppm 1.69-1.71 (m, 3H), 4.00-4.06 (m, 1H), 7.37 (d, J=8.4 Hz, 1H), 7.85-7.88 (m, 1H), 8.64-8.65 (m, 1H).

Step 2′. Preparation of tert-butyl 4-(5-bromopyridin-2-yl)-4-cyanopentanoate 4′: To a solution of 2-(5-bromopyridin-2-yl)propanenitrile 2′ (3.5 g, 16.2 mmol) in 1,4-dioxane (35 mL) was added tert-butyl acrylate 3′ (3.11 g, 23.31 mmol). The resulting reaction mixture was stirred at room temperature for 30 min. Benzyltrimethylammonium hydroxide (0.271 g, 0.295 mL, 1.620 mmol) was added, and the reaction mixture was stirred again at the same temperature for 16 h. The reaction mixture was diluted with ethyl acetate (50 mL) and extracted with water (50 mL). The organic layer was dried over sodium sulphate, filtered, and concentrated under reduced pressure to obtain crude tert-butyl 4-(5-bromopyridin-2-yl)-4-cyanopentanoate 4′ (5.96 g, 95% yield) as a yellow liquid. It was used for the next step without further purification. UPLC method 2: retention time: 1.606 min, 98.4% purity at 220 nm, [M+H−56]⁺=283.0 and [M+2+H−56]⁺=285.0. 1 H NMR (400 MHz, CDCl₃): δ ppm 1.43 (s, 9H), 1.58 (s, 3H), 2.07-2.15 (m, 1H), 2.23-2.29 (m, 1H), 2.39-2.45 (m, 2H), 7.51-7.53 (m, 1H), 7.86-7.89 (m, 1H), 8.68-8.69 (m, 1H).

Step 3′. Preparation of tert-butyl 4-(6-(5-(tert-butoxy)-2-cyano-5-oxopentan-2-yl)pyridin-3-yl)piperazine-1-carboxylate 6′: To a solution of tert-butyl 4-(5-bromopyridin-2-yl)-4-cyanopentanoate 4′ (1.5 g, 3.89 mmol) in 1,4-dioxane (20.0 mL) was added tert-butyl piperazine-1-carboxylate 5′ (0.942 g, 6.50 mmol) followed by Pd₂(dba)₃ (214 mg, 0.233 mmol) and xantphos (270 mg, 0.467 mmol). The resulting reaction mixture was stirred at 100° C. while purged with nitrogen gas for 5 min. Cs₂CO₃ (3170 mg, 9.73 mmol) was added to the reaction mixture at the same temperature and stirred at 100° C. for 16 h. The reaction mixture was cooled to room temperature, diluted with ethyl acetate (35 mL), filtered through celite, and concentrated under reduced pressure to provide the crude compound. The crude compound was purified by column chromatography using silica gel (230-400 mesh) with 20% ethyl acetate/pet ether to obtain tert-butyl 4-(6-(5-(tert-butoxy)-2-cyano-5-oxopentan-2-yl)pyridin-3-yl)piperazine-1-carboxylate 6′ (1.8 g, 86% yield). LCMS method 1: retention time: 3.412 min, 83.5% purity at 220 nm, [M+H]⁺=445.2.

Step 4′. Preparation of 3-methyl-3-(5-(piperazin-1-yl)pyridin-2-yl)piperidine-2,6-dione 7″: To a stirred solution of tert-butyl 4-(6-(5-(tert-butoxy)-2-cyano-5-oxopentan-2-yl)pyridin-3-yl)piperazine-1-carboxylate 6′ (2.95 g, 5.71 mmol) in AcOH (25.0 mL) was added H₂SO₄ (0.61 mL, 11.41 mmol). The resulting reaction mixture was heated to 120° C. and stirred for 2 h. The reaction mixture was concentrated under reduced pressure to obtain 3-methyl-3-(5-(piperazin-1-yl)pyridin-2-yl)piperidine-2,6-dione 7′ (3.5 g, 85% yield) as a brown color gum. It was used for the next step without further purification. UPLC method 2: retention time: 0.38 min, [M+H]⁺=289.2.

Step 5′. Preparation of tert-butyl 4-(6-(3-methyl-2,6-dioxopiperidin-3-yl)pyridin-3-yl)piperazine-1-carboxylate 8′: To a stirred solution of 3-methyl-3-(5-(piperazin-1-yl)pyridin-2-yl)piperidine-2,6-dione 7′ (3.5 g, 8.13 mmol) in acetonitrile (25 mL) at 0° C. was added DIPEA (1.42 mL, 8.13 mmol) and stirred for 5 min. Then di-cert-butyl dicarbonate (2.266 mL, 9.76 mmol) was added to the reaction mixture, and the resulting reaction mixture was stirred at room temperature for 4 h. The crude product was quenched with cold water (20 mL) and extracted with ethyl acetate (25 mL). The organic layer was washed with brine, dried over sodium sulphate, filtered, and concentrated under reduced pressure to obtain the crude compound. The crude compound was then purified by column chromatography using silica gel (230-400 mesh) with 62% ethyl acetate/pet ether to obtain tert-butyl 4-(6-(3-methyl-2,6-dioxopiperidin-3-yl)pyridin-3-yl)piperazine-1-carboxylate 8′ (732 mg, 22.48% yield) as an off-white solid. LCMS method 1: retention time: 2.066 min, 96.7% purity at 220 nm, [M+H]⁺=389.2. ¹H NMR (400 MHz, DMSO d6): δ ppm 1.42 (s, 9H), 1.46 (s, 3H), 1.98-2.17 (m, 2H), 2.31-2.45 (m, 2H), 3.12-3.18 (m, 4H), 3.43-3.48 (m, 4H), 7.24-7.27 (m, 1H), 7.37-7.40 (m, 1H), 8.22 (d, J=2.8 Hz, 1H), 10.77 (s, 1H).

Step 6′. SFC separation of tert-butyl 4-(6-(3-methyl-2,6-dioxopiperidin-3-yl)pyridin-3-yl)piperazine-1-carboxylate 9a′ (Peak 1) and 9b′ (Peak 2): 1.01 g of the material 8′ was purified by chiral SFC to obtain tert-butyl 4-(6-(3-methyl-2,6-dioxopiperidin-3-yl)pyridin-3-yl)piperazine-1-carboxylate (peak 1) 9a′ (0.405 g) and tert-butyl 4-(6-(3-methyl-2,6-dioxopiperidin-3-yl)pyridin-3-yl)piperazine-1-carboxylate (peak 2) 9b′ (0.396 g). SFC Method: CHIRALPAK AD-H, 250×4.6 mm, 5.0 Flow: 3.0 mL/min. Co-Solvent: 30.0% IPA. tert-butyl 4-(6-(3-methyl-2,6-dioxopiperidin-3-yl)pyridin-3-yl)piperazine-1-carboxylate (peak 1) 9a′: RT 4.18; ee 100%. LCMS method 1: retention time: 2.063 min, 98.8% purity at 220 nm, [M+H]⁺=389.2. ¹H NMR (400 MHz, DMSO d6): δ ppm 1.42 (s, 9H), 1.46 (s, 3H), 1.98-2.17 (m, 2H), 2.35-2.46 (m, 2H), 3.12-3.18 (m, 4H), 3.42-3.48 (m, 4H), 7.25-7.27 (m, 1H), 7.37-7.40 (m, 1H), 8.22 (d, J=2.8 Hz, 1H), 10.77 (s, 1H). tert-butyl 4-(6-(3-methyl-2,6-dioxopiperidin-3-yl)pyridin-3-yl)piperazine-1-carboxylate (peak 2) 9b′: RT 5.376; ee 100%; LCMS method 1: retention time: 1.888 min, 99.4% purity at 220 nm, [M+H]⁺=389.2.

¹H NMR (400 MHz, DMSO d6): δ ppm 1.42 (s, 9H), 1.46 (s, 3H), 1.98-2.17 (m, 2H), 2.11-2.44 (m, 2H), 3.14-3.18 (m, 4H), 3.45-3.47 (m, 4H), 7.24-7.27 (m, 1H), 7.37-7.40 (m, 1H), 8.22 (d, J=2.8 Hz, 1H), 10.77 (s, 1H).

Step 7′. Preparation of 3-methyl-3-(5-(piperazin-1-yl)pyridin-2-yl)piperidine-2,6-dione·2HCl (C-90a): To a stirred solution of tert-butyl 4-(6-(3-methyl-2,6-dioxopiperidin-3-yl)pyridin-3-yl)piperazine-1-carboxylate (peak 1) 9a′ (0.405 g, 1.04 mmol) in DCM (5 mL) at ° C. was added 4.0 N HCl in 1,4-dioxane (1.3 mL, 5.21 mmol). The resulting reaction mixture was stirred at room temperature for 3 h. The reaction mixture was concentrated under reduced pressure to obtain crude 3-methyl-3-(5-(piperazin-1-yl)pyridin-2-yl)piperidine-2,6-dione·2HCl (C-90a) (300 mg, 89% yield) as an off-white solid. It was used for the next step without further purification. ¹H NMR (400 MHz, DMSO d6): δ ppm 1.48 (s, 3H), 1.98-2.17 (m, 2H), 2.37-2.41 (m, 1H), 3.21-3.25 (m, 4H), 3.40-3.43 (m, 4H), 7.30-7.32 (m, 1H), 7.43-7.46 (m, 1H), 8.27 (d, J=2.8 Hz, 1H), 8.84 (brs, 1H), 10.79 (s, 1H).

Step 8′. Preparation of 3-methyl-3-(5-(piperazin-1-yl)pyridin-2-yl)piperidine-2,6-dione·2HCl (C-90b): To a stirred solution of tert-butyl 4-(6-(3-methyl-2,6-dioxopiperidin-3-yl)pyridin-3-yl)piperazine-1-carboxylate (peak 2) 9b′ (0.39 g, 1.00 mmol) in DCM (4 mL) at ° C. was added 4.0 N HCl in 1,4-dioxane (2.5 mL, 10.04 mmol). The resulting reaction mixture was stirred at room temperature for 2 h. The reaction mixture was concentrated under reduced pressure to obtain crude 3-methyl-3-(5-(piperazin-1-yl)pyridin-2-yl)piperidine-2,6-dione·2HCl (C-90b) (340 mg, 86% yield) as an off-white solid. It was used for the next step without further purification. LCMS method 1: retention time: 0.874 min, 92.0% purity at 220 nm, [M+H]⁺=289.2. ¹H NMR (400 MHz, DMSO d₆): δ ppm 1.50 (s, 3H), 1.99-2.07 (m, 1H), 2.11-2.21 (m, 1H), 2.32-2.45 (m, 2H), 3.19-3.23 (m, 4H), 3.45-3.48 (m, 4H), 7.35-7.37 (m, 1H), 7.51-7.53 (m, 1H), 8.28 (d, J=2.8 Hz, 1H), 9.21 (s, 2H), 10.83 (s, 1H).

### Example S63: Preparation of (R or S)-3-(6-((3aR,6aR or 3aS, 6a5)-hexahydropyrrolo[3,2-b]pyrrol-1(2H)-yl)pyridin-3-yl)-3-methylpiperidine-2,6-dione (C-93a), (R or S)-3-(6-((3aR,6aR or 3aS, 6a5)-hexahydropyrrolo[3,2-b]pyrrol-1(2H)-yl)pyridin-3-yl)-3-methylpiperidine-2,6-dione (C-93b)

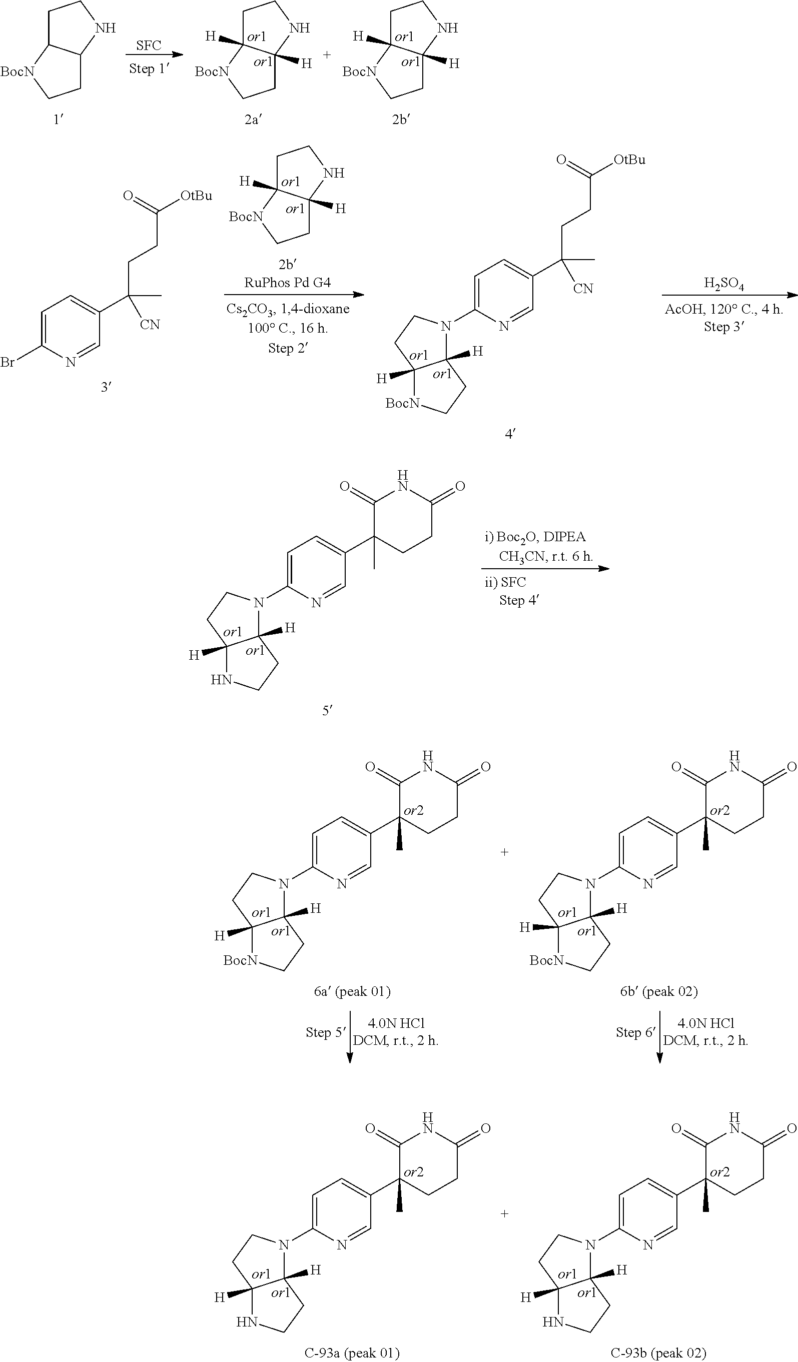

LCMS Method 1. Kinetex XB-C18, 50×4.6 mm, 5.0 Temperature: RT, Flow: 1.0 mL/min, run time: 5.5 min. Mobile Phase Conditions: Mobile Phase-A: 0.1% TFA in H₂O, Mobile Phase-B: 0.1% TFA in ACN, Gradient: Initial 95% Mobile Phase A and 5% Mobile Phase B linear gradient to 95% Mobile Phase B for 2.5 min. MSD positive.

UPLC Method 2. Aquity BEH-C18, 50×2.1 mm, 1.7 Temperature: RT, Flow: mL/min, run time: 2.5 min. Mobile Phase Conditions: Mobile Phase-A: 0.1% TFA in H₂O, Mobile Phase-B: 0.1% TFA in ACN, Gradient: Initial 95% Mobile Phase A and 5% Mobile Phase B linear gradient to 98% Mobile Phase B for 1.5 min. MSD positive.

LCMS Method 3. Kinetex XB-C18, 75×3.0 mm, 2.6 Temperature: RT, Flow: 1.0 mL/min, run time: 5.0 min. Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 98% Mobile Phase A and 2% Mobile Phase B linear gradient to 100% Mobile Phase B for 4 min. MSD positive.

Step 1′. Chiral SFC separation of tert-butyl hexahydropyrrolo[3,2-b]pyrrole-1(2H)-carboxylate 2a″ (Peak-1) and tert-butyl hexahydropyrrolo[3,2-b]pyrrole-1(2H)-carboxylate 2b′ (Peak-2): The enantiomers of tert-butyl hexahydropyrrolo[3,2-b]pyrrole-1(2H)-carboxylate (2.0 g) were separated by chiral SFC to obtain to get Peak-1 2a′ (960 mg, mmol, 23.6% yield) as a yellow liquid and Peak-2 2b′ (930 mg, 0.313 mmol, 21.9% yield) as a yellow liquid. SFC Method: Chiralpak IG, 250 mm×4.6 mm, 5.0 Flow: 3.0 mL/min, Co-Solvent: 40.0% (0.1% NH₄OH in methanol). Peak-1: 2.27 min and Peak-2: 3.797 min tert-butyl hexahydropyrrolo[3,2-b]pyrrole-1(2H)-carboxylate 2a′ (Peak-01, RT 2.27 min, ee 100%).

LCMS method 1: retention time: 1.551 min, [M+H]⁺=213.2. tert-butyl hexahydropyrrolo[3,2-b]pyrrole-1(2H)-carboxylate 2b′ (Peak-02, RT 3.797 min; ee 100%). LCMS method 1: retention time: 1.53 min, [M+H]⁺=213.2

Step 2′. Preparation of tert-butyl 4-(5-(5-(tert-butoxy)-2-cyano-5-oxopentan-2-yl)pyridin-2-yl)hexahydropyrrolo[3,2-b]pyrrole-1(2H)-carboxylate 4″: To a stirred solution of tert-butyl 4-(6-bromopyridin-3-yl)-4-cyanopentanoate 3′ (800 mg, 2.358 mmol, 1.0 eq.) in 1,4-dioxane (10.0 mL) was added tert-butyl hexahydropyrrolo[3,2-b]pyrrole-1(2H)-carboxylate 2b′ (601 mg, 2.83 mmol, 1.2 eq.) followed by cesium carbonate (1.54 g, 4.72 mmol, 2.0 eq.). The resulting reaction mixture was degassed for 10 min with nitrogen gas, and then, RuPhos-Pd-G3 (118 mg, 0.141 mmol, 0.06 eq.) was added. The reaction mixture was heated to 90° C. and stirred for 16 h. The reaction mixture was cooled to room temperature, filtered through celite, and the filtrate was concentrated under reduced pressure to obtain the crude product. The crude product was purified by column chromatography using silica gel (230-400 mesh) with 40% ethyl acetate/pet ether to obtain tert-butyl 4-(5-(5-(tert-butoxy)-2-cyano-5-oxopentan-2-yl)pyridin-2-yl)hexahydropyrrolo[3,2-b]pyrrole-1(2H)-carboxylate 4″(700 mg, 1.12 mmol, 47.3% yield) as a yellow solid. LCMS method 3: retention time: 3.216 min, [M+H]⁺=471.2.

Step 3′. Preparation of 3-(6-(hexahydropyrrolo[3,2-b]pyrrol-1(2H)-yl)pyridin-3-yl)-3-methylpiperidine-2,6-dione 5″: To a stirred solution of tert-butyl 4-(5-(5-(tert-butoxy)-2-cyano-5-oxopentan-2-yl)pyridin-2-yl) hexahydropyrrolo[3,2-b]pyrrole-1(2H)-carboxylate 4′ (700 mg, 1.12 mmol, 1.0 eq.) in AcOH (7.0 mL) was added sulfuric acid (0.304 mL, 5.58 mmol, eq.). The resulting reaction mixture was heated to 120° C. and stirred for 3 h. The reaction mixture was concentrated under reduced pressure to obtain crude 3-(6-(hexahydropyrrolo[3,2-b]pyrrol-1(2H)-yl)pyridin-3-yl)-3-methylpiperidine-2,6-dione 5′ (450 mg) as a brown solid. It was used for the next step without further purification. UPLC method 2: retention time: 0.357 min, [M+H]⁺=315.2

Step 4′. Preparation and chiral SFC separation of tert-butyl 4-(5-(3-methyl-2,6-dioxopiperidin-3-yl)pyridin-2-yl)hexahydropyrrolo[3,2-b]pyrrole-1(2H)-carboxylate 6a″ (Peak-1) and tert-butyl 4-(5-(3-methyl-2,6-dioxopiperidin-3-yl)pyridin-2-yl)hexahydropyrrolo[3,2-b]pyrrole-1(2H)-carboxylate 6b′ (Peak-2): To a stirred solution of 3-(6-(hexahydropyrrolo[3,2-b]pyrrol-1(2H)-yl)pyridin-3-yl)-3-methylpiperidine-2,6-dione 5′ (450 mg, 1.43 mmol, 1.0 eq.) in acetonitrile (10 mL) at 0° C. was added DIPEA (1.50 mL, 8.59 mmol, 6.0 eq.) and stirred for 5 min. Then di-tert-butyl decarbonate ((0.498 mL, 2.15 mmol, 1.5 eq.) was added to the reaction mixture, and the resulting reaction mixture was stirred at RT for 16 h. Then, the reaction mixture was concentrated under reduced pressure to obtain the crude product. The crude product was purified by column chromatography using silica gel (230-400 mesh) with 40% ethyl acetate/pet ether to obtain the diastereomeric mixture of tert-butyl 4-(5-(3-methyl-2,6-dioxopiperidin-3-yl)pyridin-2-yl)hexahydropyrrolo[3,2-b]pyrrole-1(2H)-carboxylate (0.3 g, 51% yield) as an off-white solid. The diastereomers were separated by chiral SFC to obtain to get Peak-1 5a′ (140 mg, 0.338 mmol, 23.58% yield) and Peak-2 5b′ (130 mg, mmol, 21.9% yield). SFC Method: Chiralpak AD-H, 250 mm×4.6 mm, 5.0 Flow: 3.0 mL/min, Co-Solvent: 40.0% IPA.

Peak-1: 2.48 min and Peak-2: 3.285 min; tert-butyl 4-(5-(3-methyl-2,6-dioxopiperidin-3-yl)pyridin-2-yl)hexahydropyrrolo[3,2-b]pyrrole-1(2H)-carboxylate 6a″(Peak-01, RT 2.485 min, ee 99.9%): 1-H-NMR (400 MHz, DMSO-d6): δ 1.41 (m, 9H), 1.42 (s, 3H), 1.98-2.14 (m, 6H), 2.31-2.33 (m, 1H), 2.34-2.45 (m, 1H), 3.06-3.09 (m, 1H), 3.48-3.51 (m, 1H), 3.58-3.61 (m, 1H), 4.32 (brs, 1H), 4.46 (brs, 1H), 6.51 (d, J=8.8 Hz, 1H), 7.44-7.47 (m, 1H), 7.96 (dd, J=2.4 Hz and 7.2 Hz, 1H), 10.87 (s, 1H). LCMS method 3: retention time: 1.561 min, [M+H]⁺=415.2.

tert-butyl 4-(5-(3-methyl-2,6-dioxopiperidin-3-yl)pyridin-2-yl)hexahydropyrrolo[3,2-b]pyrrole-1(2H)-carboxylate 613′ (Peak-02, RT 3.285 min; ee 99.6%): 1H-NMR (400 MHz, DMSO-d6): δ 1.41 (m, 9H), 1.42 (s, 3H), 1.99-2.16 (m, 6H), 2.31-2.36 (m, 1H), 2.47-2.50 (m, 1H), 3.06-3.09 (m, 1H), 3.48-3.51 (m, 1H), 3.53-3.62 (m, 1H), 4.33 (brs, 1H), 4.47 (brs, 1H), 6.52 (d, J=8.8 Hz, 1H), 7.44-7.47 (m, 1H), 7.97 (dd, J=2.4 Hz and 7.2 Hz, 1H), 10.87 (s, 1H). LCMS method 3: retention time: 1.23 min, [M+H]⁺=415.2.

Step 5′. Preparation of 3-(6-(hexahydropyrrolo[3,2-b]pyrrol-1(2H)-yl)pyridin-3-yl)-3-methylpiperidine-2,6-dione (C-93a): To a stirred solution of tert-butyl 4-(5-(3-methyl-2,6-dioxopiperidin-3-yl)pyridin-2-yl)hexahydropyrrolo[3,2-b]pyrrole-1(2H)-carboxylate Peak-1 6a′ (140 mg, 0.334 mmol, 1.0 eq.) in DCM (1.0 mL) at 0° C. was added 4.0 N HCl in 1,4-dioxane (0.203 mL, 6.69 mmol, 20.0 eq.). The resulting reaction mixture was stirred at RT for 2 h. The reaction mixture was concentrated under reduced pressure to obtain the crude product, and it was washed with diethyl ether (2×15.0 mL) to get 3-(6-(hexahydropyrrolo[3,2-b]pyrrol-1(2H)-yl)pyridin-3-yl)-3-methylpiperidine-2,6-dione 7a′ (95 mg, 0.297 mmol, 89% yield). It was used for the next step without further purification. LCMS method 1: retention time: 1.454 min, 98.23% purity at 220 nm, [M+H]⁺=315.2.

Step 6′. Preparation of 3-(6-(hexahydropyrrolo[3,2-b]pyrrol-1(2H)-yl)pyridin-3-yl)-3-methylpiperidine-2,6-dione (C-93b): To a stirred solution of tert-butyl 4-(5-(3-methyl-2,6-dioxopiperidin-3-yl)pyridin-2-yl)hexahydropyrrolo[3,2-b]pyrrole-1(2H)-carboxylate Peak-2 6b′ (130 mg, 0.314 mmol, 1.0 eq.) in DCM (2.0 mL)) at 0° C. was added 4.0 N HCl in HCl in 1,4-dioxane (0.095 mL, 3.14 mmol, 10.0 eq.). The resulting reaction mixture was stirred at RT for 2 h. The reaction mixture was concentrated under reduced pressure to obtain a crude product and it was washed with diethyl ether (2×15.0 mL) to get 3-(6-(hexahydropyrrolo[3,2-b]pyrrol-1(2H)-yl)pyridin-3-yl)-3-methylpiperidine-2,6-dione 7b′ (200 mg). It was used for the next step without further purification.

LCMS method 1: retention time: 1.014 min, [M+H]⁺=315.2

### Example S64. Preparation of (R or S)-3-(6-((3aR,6aR or 3aS, 6a5)-hexahydropyrrolo[3,2-b]pyrrol-1(2H)-yl)pyridin-3-yl)-3-methylpiperidine-2,6-dione (C-93c), (R or S)-3-(6-((3aR,6aR or 3aS, 6a5)-hexahydropyrrolo[3,2-b]pyrrol-1(2H)-yl)pyridin-3-yl)-3-methylpiperidine-2,6-dione (C-93d)

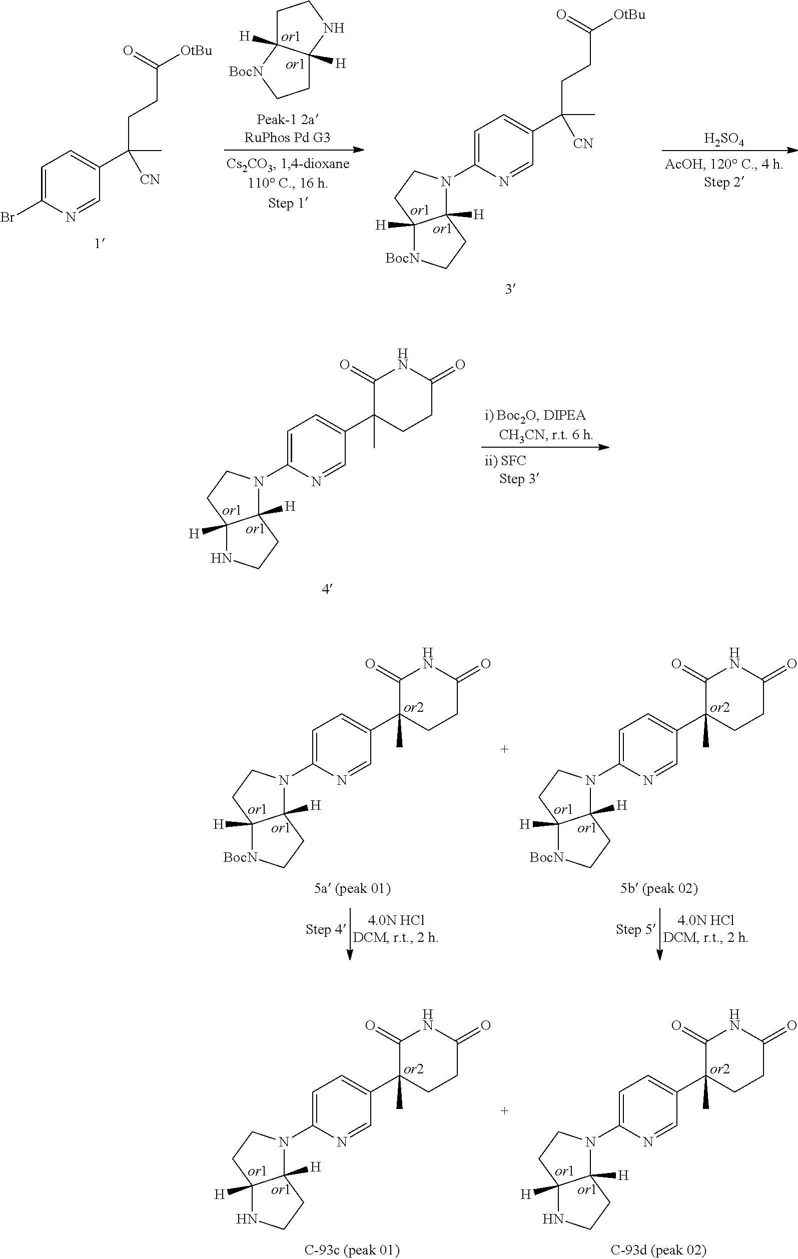

LCMS Method 1. Kinetex XB-C18, 50×4.6 mm, 5.0 Temperature: RT, Flow: 1.0 mL/min, run time: 5.5 min. Mobile Phase Conditions: Mobile Phase-A: 0.1% TFA in H₂O,

Mobile Phase-B: 0.1% TFA in ACN, Gradient: Initial 95% Mobile Phase A and 5% Mobile Phase B linear gradient to 95% Mobile Phase B for 2.5 min. MSD positive.

UPLC Method 2. Aquity BEH-C18, 50×2.1 mm, 1.7 Temperature: RT, Flow: mL/min, run time: 2.5 min. Mobile Phase Conditions: Mobile Phase-A: 0.1% TFA in H₂O, Mobile Phase-B: 0.1% TFA in ACN, Gradient: Initial 80% Mobile Phase A and 20% Mobile Phase B linear gradient to 98% Mobile Phase B for 1.5 min. MSD positive.

LCMS Method 3. Kinetex XB-C18, 75×3.0 mm, 2.6 Temperature: RT, Flow: 1.0 mL/min, run time: 5.0 min. Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 98% Mobile Phase A and 2% Mobile Phase B linear gradient to 100% Mobile Phase B for 4 min. MSD positive.

Step 1′. Preparation of tert-butyl 4-(6-(5-(tert-butoxy)-2-cyano-5-oxopentan-2-yl)pyridin-3-yl)hexahydropyrrolo[3,2-b]pyrrole-1(2H)-carboxylate 3″: To a stirred solution of tert-butyl 4-(5-bromopyridin-2-yl)-4-cyanopentanoate 1′ (750 mg, 2.03 mmol, 1.0 eq.) and tert-butyl hexahydropyrrolo[3,2-b]pyrrole-1(2H)-carboxylate 2a′ (475 mg, 2.24 mmol, 1.1 eq.) in 1,4-dioxane (10 mL) was added cesium carbonate (1.98 g, 6.10 mmol, 3.0 eq.). The resulting reaction mixture was degassed for 10 min with nitrogen, and then RuPhos Pd G3 (102 mg, 0.122 mmol, 0.06 eq.) was added. The reaction mixture was heated to 100° C. and stirred for 16 h. The reaction mixture was cooled to room temperature, quenched the reactant mixture with water (10.0 mL), and extracted with ethyl acetate (3×20 mL). The organic layer was washed with brine, dried over sodium sulphate, and distilled under reduced pressure to obtain the crude product. The crude product was purified by column chromatography using silica gel (230-400 mesh) with 30% ethyl acetate/pet ether to obtain tert-butyl 4-(6-(5-(tert-butoxy)-2-cyano-5-oxopentan-2-yl)pyridin-3-yl)hexahydropyrrolo[3,2-b]pyrrole-1(2H)-carboxylate 3′ (660 mg, 1.234 mmol, 60.7% yield) as a colorless gummy liquid. LCMS method 1: retention time: 2.78 min, [M+H]⁺=471.3.

Step 2′. Preparation of 3-(5-(hexahydropyrrolo[3,2-b]pyrrol-1(2H)-yl)pyridin-2-yl)-3-methylpiperidine-2,6-dione 4′: To a stirred solution of tert-butyl 4-(6-(5-(tert-butoxy)-2-cyano-5-oxopentan-2-yl)pyridin-3-yl)hexahydropyrrolo[3,2-b]pyrrole-1(2H)-carboxylate 3′ (700 mg, 1.487 mmol, 1.0 eq.) in AcOH (7.0 mL) was added H₂SO₄ (0.159 mL, 2.97 mmol, 2.0 eq.). The resulting reaction mixture was heated to 120° C. and stirred for 4 h. The reaction mixture was concentrated under reduced pressure to 3-(5-(hexahydropyrrolo[3,2-b]pyrrol-1(2H)-yl)pyridin-2-yl)-3-methylpiperidine-2,6-dione 4′ (1.15 g) as a brown color gum. It was used for the next step without further purification.

UPLC method 2: retention time: 0.348 min, [M+H]⁺=315.4.

Step 3′. Preparation and chiral SFC separation of tert-butyl 4-(6-(3-methyl-2,6-dioxopiperidin-3-yl)pyridin-3-yl)hexahydropyrrolo[3,2-b]pyrrole-1(2H)-carboxylate 5a′ and 5b′: To a stirred solution of 3-(5-(hexahydropyrrolo[3,2-b]pyrrol-1(2H)-yl)pyridin-2-yl)-3-methylpiperidine-2,6-dione 4′ (1.1 g, 3.39 mmol, 1.0 eq.) in acetonitrile (5 mL) at 0° C. was added DIPEA (1.78 mL, 10.2 mmol, 3.0 eq.) and stirred for 5 min. Then di-tert-butyl decarbonate (1.182 mL, 5.09 mmol, 1.5 eq.) was added to the reaction mixture, and the resulting reaction mixture was stirred at RT for 16 h. Then, the reaction mixture was concentrated under reduced pressure to obtain the crude product. Obtained crude was diluted with water (10 mL) and extracted with ethyl acetate (3×15 mL). The combined organic layers were washed with brine solution, dried over sodium sulphate, and evaporated the solvent was under reduced pressure to get crude compound. The crude product was purified by column chromatography using silica gel (230-400 mesh) with 10% ethyl acetate/pet ether to obtain the crude product as a mixture of two diastereomers. Both the diastereomers were separated by chiral SFC to get 5a′ (115 mg, 0.275 mmol, 8.09% yield, Peak-01, Rt 2.48) and 5b′ (130 mg, 0.310 mmol, 9.15% yield, Peak-02, Rt 3.093). SFC Method: Chiralpak AD-H, 250 mm×4.6 mm, 5.0 Flow: 3.0 mL/min, Co-Solvent: 40.0% IPA. tert-butyl 4-(6-(3-methyl-2,6-dioxopiperidin-3-yl)pyridin-3-yl)hexahydropyrrolo[3,2-b]pyrrole-1(2H)-carboxylate Peak-1 5a″(RT 2.48, ee 100%): (400 MHz, DMSO-d6): δ ppm 1.43 (m, 9H), 1.44 (s, 3H), 1.86-2.16 (m, 6H), 2.33-2.43 (m, 2H), 3.06-3.09 (m, 1H), 3.35-3.38 (m, 1H), 3.46-3.51 (m, 1H), 4.34 (m, 2H), 6.99 (dd, J=3.2 Hz and 8.8 Hz, 1H), 7.20 (d, J=8.8 Hz, 1H), 7.90 (d, J=2.8 Hz, 1H), 10.73 (s, 1H). LCMS Method 3: retention time: 2.45 min, 99.59% purity at 220 nm, [M+H]⁺=415.2. tert-butyl 4-(6-(3-methyl-2,6-dioxopiperidin-3-yl)pyridin-3-yl)hexahydropyrrolo[3,2-b]pyrrole-1(2H)-carboxylate Peak-2 513″(RT 3.093; ee 99.5%): 1H-NMR (400 MHz, DMSO-d6): δ ppm 1.43 (m, 9H), 1.44 (s, 3H), 1.83-1.99 (m, 1H), 2.01-2.16 (m, 5H), 2.33-2.43 (m, 2H), 3.07-3.09 (m, 1H), 3.35-3.37 (m, 1H), 3.48-3.50 (m, 1H), 4.33-4.35 (m, 2H), 6.99 (dd, J=3.2 Hz and 8.8 Hz, 1H), 7.20 (d, J=8.4 Hz, 1H), 7.90 (d, J=2.8 Hz, 1H), 10.73 (s, 1H). LCMS Method 3: retention time: 2.453 min, 99.85% purity at 220 nm, [M+H]⁺=415.2.

Step 4′. Preparation of 3-(5-(hexahydropyrrolo[3,2-b]pyrrol-1(2H)-yl)pyridin-2-yl)-3-methylpiperidine-2,6-dione·2HCl (C-93c): To a stirred solution of tert-butyl 4-(6-(3-methyl-2,6-dioxopiperidin-3-yl)pyridin-3-yl)hexahydropyrrolo[3,2-b]pyrrole-1(2H)-carboxylate Peak-1 5a′ (110 mg, 0.265 mmol, 1.0 eq.) in DCM (2.0 mL) at 0° C. was added 4.0 M HCl in 1,4-dioxane (0.081 mL, 2.65 mmol, 10 eq.). The resulting reaction mixture was stirred at RT for 2 h. The reaction mixture was concentrated under reduced pressure to obtain crude 3-(5-(hexahydropyrrolo[3,2-b]pyrrol-1(2H)-yl)pyridin-2-yl)-3-methylpiperidine-2,6-dione·2HCl (C-93c) (180 mg). It was used for the next step without further purification. LCMS method 3: retention time: 1.42 min, [M+H]⁺=315.2.

Step 5′. Preparation of. (5-(hexahydropyrrolo[3,2-b]pyrrol-1(2H)-yl)pyridin-2-yl)-3-methylpiperidine-2,6-dione (C-93d): To a stirred solution of tert-butyl 4-(6-(3-methyl-2,6-dioxopiperidin-3-yl)pyridin-3-yl)hexahydropyrrolo[3,2-b]pyrrole-1(2H)-carboxylate Peak-2 (140 mg, 0.338 mmol, 1.0 eq.) in DCM (1 mL) was added 4.0 M HCl in 1,4-dioxane (0.258 mL, 8.49 mmol, 20 eq.) at RT. The resulting solution was stirred for 2 h at RT. The reaction mixture was then concentrated under reduced pressure to give crude product. Crude product was washed with diethyl ether to get 3-(5-(hexahydropyrrolo[3,2-b]pyrrol-1(2H)-yl)pyridin-2-yl)-3-methylpiperidine-2,6-dione (C-93d) (90 mg). It was used for the next step without further purification. LCMS method 1: retention time 1.143 min, [M+H]⁺=315.2.

Table 53 summarizes CBM intermediates prepared by a General Procedure similar to Example S62. All intermediates are enantiomerically pure single diastereomers.

TABLE 53
CBM intermediates prepared via General Procedure
similar to Example S62.

| Intermediate | |
|---|---|
| No. | Structure |
| C-91a (peak 2 of cyclopentane separation; peak 1 of quat methyl separation) | 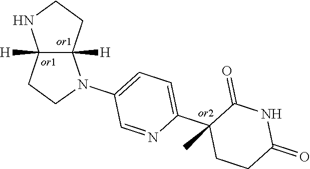 [SMILES: C[C@]1(c2ccc(N3CC[C@@H]4NCC[C@@H]43)cn2)CCC(=O)NC1=O] |
| C-91b (peak 2 of cyclopentane separation; peak 2 of quat methyl separation) | 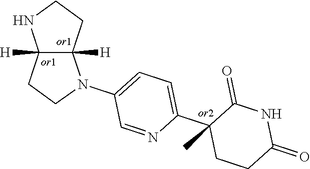 [SMILES: C[C@]1(c2ccc(N3CC[C@@H]4NCC[C@@H]43)cn2)CCC(=O)NC1=O] |
| C-91c (peak 1 of cyclopentane separation; peak 1 of quat methyl separation) | 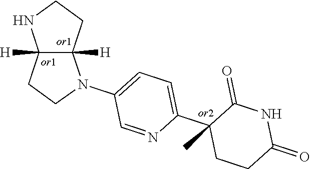 [SMILES: C[C@]1(c2ccc(N3CC[C@@H]4NCC[C@@H]43)cn2)CCC(=O)NC1=O] |
| C-91d (peak 1 of cyclopentane separation; peak 2 of quat methyl separation) | 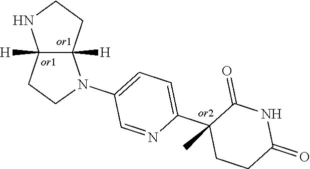 [SMILES: C[C@]1(c2ccc(N3CC[C@@H]4NCC[C@@H]43)cn2)CCC(=O)NC1=O] |
| C-92a (peak 1) | 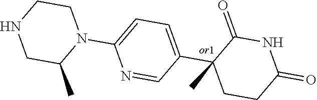 [SMILES: C[C@H]1CNCCN1c1ccc([C@@]2(C)CCC(=O)NC2=O)cn1] |
| C-92b (peak 2) | 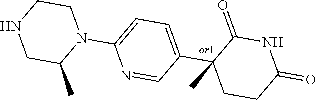 [SMILES: C[C@H]1CNCCN1c1ccc([C@@]2(C)CCC(=O)NC2=O)cn1] |
| C-94a (peak 1) | 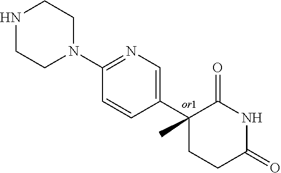 [SMILES: C[C@]1(c2ccc(N3CCNCC3)nc2)CCC(=O)NC1=O] |
| C-94b (peak 2) | 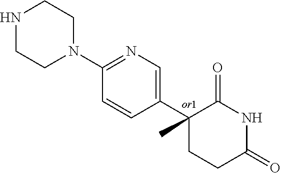 [SMILES: C[C@]1(c2ccc(N3CCNCC3)nc2)CCC(=O)NC1=O] |
| C-95a (peak 1) | 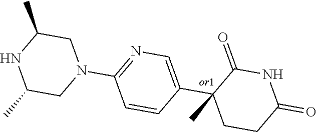 [SMILES: C[C@H]1CN(c2ccc([C@@]3(C)CCC(=O)NC3=O)cn2)C[C@H](C)N1] |
| C-95b (peak 2) | 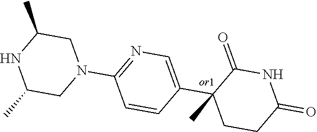 [SMILES: C[C@H]1CN(c2ccc([C@@]3(C)CCC(=O)NC3=O)cn2)C[C@H](C)N1] |
| C-96a (peak 1) | 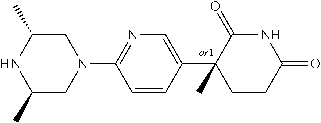 [SMILES: C[C@@H]1CN(c2ccc([C@@]3(C)CCC(=O)NC3=O)cn2)C[C@@H](C)N1] |
| C-96b (peak 2) | 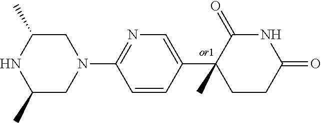 [SMILES: C[C@@H]1CN(c2ccc([C@@]3(C)CCC(=O)NC3=O)cn2)C[C@@H](C)N1] |
| C-97a (peak 1) | 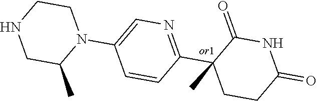 [SMILES: C[C@H]1CNCCN1c1ccc([C@@]2(C)CCC(=O)NC2=O)nc1] |
| C-97b (peak 2) | 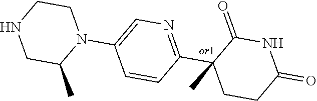 [SMILES: C[C@H]1CNCCN1c1ccc([C@@]2(C)CCC(=O)NC2=O)nc1] |
| C-98a (peak 1) | 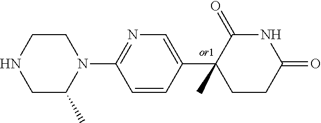 [SMILES: C[C@@H]1CNCCN1c1ccc([C@@]2(C)CCC(=O)NC2=O)cn1] |
| C-98b (peak 2) | 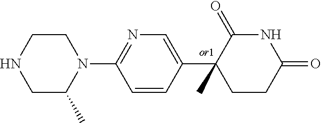 [SMILES: C[C@@H]1CNCCN1c1ccc([C@@]2(C)CCC(=O)NC2=O)cn1] |
| C-99a (peak 1) | 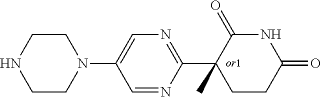 [SMILES: C[C@]1(c2ncc(N3CCNCC3)cn2)CCC(=O)NC1=O] |
| C-99b (peak 2) | 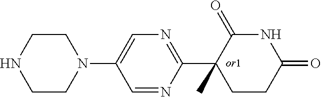 [SMILES: C[C@]1(c2ncc(N3CCNCC3)cn2)CCC(=O)NC1=O] |
| C-100a (peak 1) | 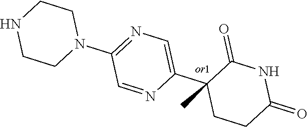 [SMILES: C[C@]1(c2cnc(N3CCNCC3)cn2)CCC(=O)NC1=O] |
| C-100b (peak 2) | 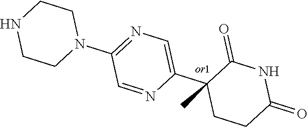 [SMILES: C[C@]1(c2cnc(N3CCNCC3)cn2)CCC(=O)NC1=O] |
| C-101a (peak 1) | 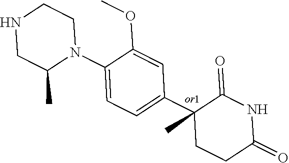 [SMILES: COc1cc([C@@]2(C)CCC(=O)NC2=O)ccc1N1CCNC[C@@H]1C] |
| C-101b (peak 2) | 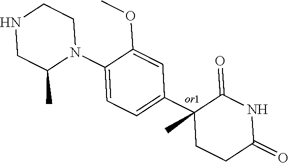 [SMILES: COc1cc([C@@]2(C)CCC(=O)NC2=O)ccc1N1CCNC[C@@H]1C] |
| C-102a (peak 1) | 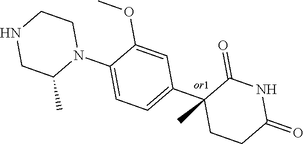 [SMILES: COc1cc([C@@]2(C)CCC(=O)NC2=O)ccc1N1CCNC[C@H]1C] |
| C-102b (peak 2) | 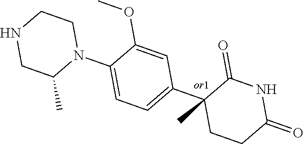 [SMILES: COc1cc([C@@]2(C)CCC(=O)NC2=O)ccc1N1CCNC[C@H]1C] |
| C-102a (peak 1) | 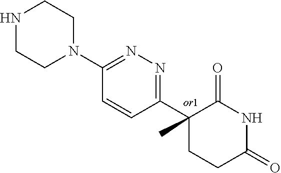 [SMILES: C[C@]1(c2ccc(N3CCNCC3)nn2)CCC(=O)NC1=O] |
| C-102b (peak 2) | 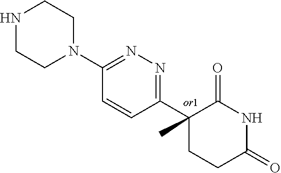 [SMILES: C[C@]1(c2ccc(N3CCNCC3)nn2)CCC(=O)NC1=O] |
| C-103a (peak 1) | 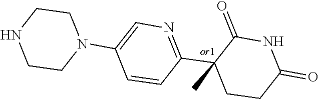 [SMILES: C[C@]1(c2ccc(N3CCNCC3)cn2)CCC(=O)NC1=O] |
| C-103b (peak 2) | 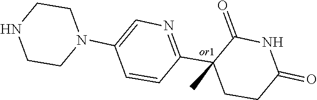 [SMILES: C[C@]1(c2ccc(N3CCNCC3)cn2)CCC(=O)NC1=O] |

### Example S65. Preparation of 3-(1-(piperidin-4-yl)-1H-pyrazol-4-yl)piperidine-2,6-dione (C-104)

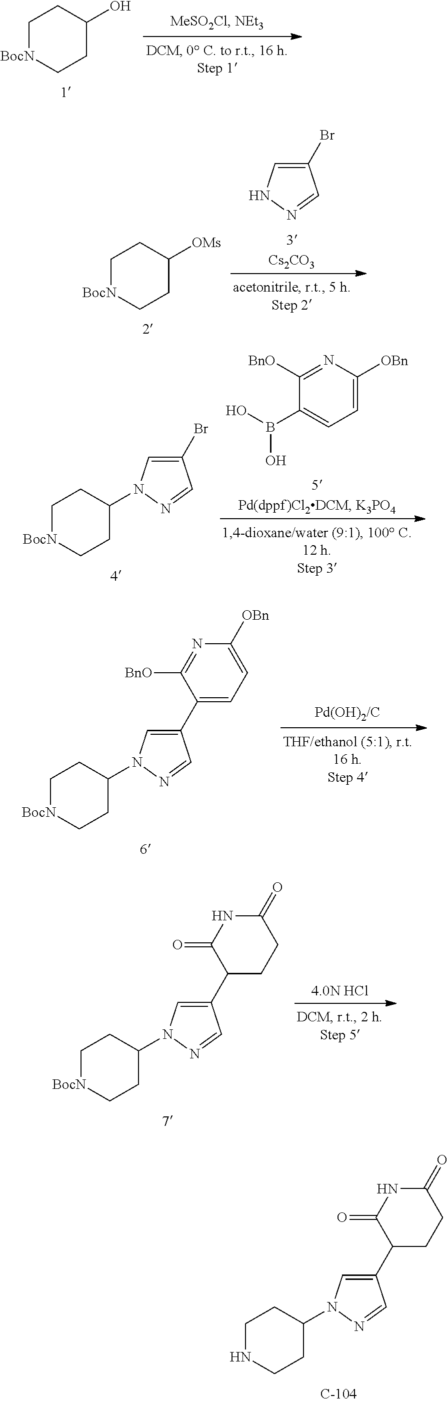

LCMS Method 1. Kinetex XB-C18, 75×3.0 mm, 2.6 Temperature: RT, Flow: 1.0 mL/min, run time: 5.0 min. Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 98% Mobile Phase A and 2% Mobile Phase B linear gradient to 100% Mobile Phase B for 4 min. MSD positive.

UPLC Method 2. Aquity BEH-C18, 50×2.1 mm, 1.7 Temperature: RT, Flow: mL/min, run time: 2.5 min. Mobile Phase Conditions: Mobile Phase-A: 0.1% TFA in H₂O, Mobile Phase-B: 0.1% TFA in ACN, Gradient: Initial 95% Mobile Phase A and 5% Mobile Phase B linear gradient to 98% Mobile Phase B for 1.5 min. MSD positive.

Step 1′. Synthesis of tert-butyl 4-((methylsulfonyl)oxy)piperidine-1-carboxylate 2′. To a stirred solution of tert-butyl 4-hydroxypiperidine-1-carboxylate 1″(1.0 g, 4.97 mmol) in DCM (10 mL) was added triethyl amine (0.693 mL, 4.97 mmol) and methanesulfonyl chloride (0.569 g, 4.97 mmol) at 0° C. The reaction mixture was stirred at room temperature for 16 h. The reaction mixture was treated with water (20 mL) and extracted with ethyl acetate (3×50 mL). The organic layer was washed with brine (20 mL). The combined organic extracts were dried over sodium sulphate, filtered, and concentrated under reduced pressure to obtain the crude tert-butyl 4-((methylsulfonyl)oxy)piperidine-1-carboxylate 2′ (1.62 g) as a yellow liquid. It was used for the next step without further purification. ¹H NMR (400 MHz, DMSO d6): δ ppm 1.40 (s, 9H), 1.56-1.65 (m, 2H), 1.88-1.95 (m, 2H), 3.16-3.21 (m, 5H), 3.58-3.64 (m, 2H), 4.80-4.86 (m, 1H).

Step 2′. Synthesis of tert-butyl 4-(4-bromo-1Hpyrazol-1-yl)piperidine-1-carboxylate 4′. To a stirred solution of 4-bromo-1H-pyrazole 3′ (0.55 g, 3.74 mmol) in acetonitrile (10 mL) was added cesium carbonate (2.439 g, 7.48 mmol). The resulting reaction mixture was stirred at room temperature for 10 min. tert-butyl 4-((methylsulfonyl)oxy)piperidine-1-carboxylate 2′ (1.4 g, 5.01 mmol) was added, and the reaction mixture was stirred again at the same temperature for 16 h. The reaction mixture was treated with water and extracted with ethyl acetate (3×30 mL). The combined organic extras were washed with brine (30 mL), dried over sodium sulphate, filtered, and concentrated under reduced pressure to obtain a crude compound. The crude compound was purified by column chromatography using silica gel (230-400 mesh) with 30% ethyl acetate/pet ether to obtain tert-butyl 4-(4-bromo-1Hpyrazol-1-yl)piperidine-1-carboxylate 4′ (0.732 g, 51.5% yield) as a yellow solid. LCMS method 1: retention time: 2.506 min, [M+H]⁺=230.0 and [M+2+H]⁺=232.0. ¹H NMR (400 MHz, CDCl₃): δ ppm 1.42 (s, 9H), 1.69-1.80 (m, 2H), 1.96-1.99 (m, 2H), 2.85-2.92 (m, 2H), 4.01-4.08 (m, 2H), 4.31-4.39 (m, 1H), 7.55 (s, 1H), 8.06 (s, 1H).

Step 3′. Preparation of tert-butyl 4-(4-(2,6-bis(benzyloxy)pyridin-3-yl)-1H-pyrazol-1-yl)piperidine-1-carboxylate 6′. To a stirred solution of tert-butyl 4-(4-bromo-1H-pyrazol-1-yl)piperidine-1-carboxylate 4′ (0.7 g, 1.84 mmol) in 1,4-dioxane (9 mL) and water (1 mL) was added (2,6-bis(benzyloxy)pyridin-3-yl)boronic acid 5′ (0.680 g, 2.029 mmol) and K₃PO₄ (1.17 g, 5.53 mmol). The reaction mixture was degassed with nitrogen, followed by Pd(dppf)Cl₂·DCM (0.151 g, 0.184 mmol) was added. The reaction mixture was stirred at 100° C. for 12 h. The reaction mixture was quenched with water (30 mL) and extracted using ethyl acetate (3×30 mL). The combined organic extracts were washed with brine (20 mL), dried over sodium sulphate, filtered, and concentrated under reduced pressure to obtain the crude compound. The crude compound was purified by column chromatography using silica gel (230-400 mesh) with 30% ethyl acetate/pet ether to obtain tert-butyl 4-(4-(2,6-bis(benzyloxy)pyridin-3-yl)-1H-pyrazol-1-yl)piperidine-1-carboxylate 6′ (0.345 g, 32.5% yield) as an off-white solid. LCMS method 1: retention time: 1.638 min, [M+H]⁺=541.3.

Step 4′. Synthesis of tert-butyl 4-(4-(2,6-dioxopiperidin-3-yl)-1H-pyrazol-1-yl)piperidine-1-carboxylate 7′. To a stirred solution of tert-butyl 4-(4-(2,6-bis(benzyloxy)pyridin-3-yl)-1H-pyrazol-1-yl)piperidine-1-carboxylate 6′ (0.343 g, 0.174 mmol) in ethanol (1 mL) and THF (5 mL) was added Pd(OH)₂/C (89.0 mg, 0.634 mmol). The reaction mixture was stirred for 16 h at room temperature under H₂ atmosphere. The reaction mixture was filtered through a celite bed, and the celite bed was washed with THF. The filtrate was concentrated under reduced pressure to obtain crude tert-butyl 4-(4-(2,6-dioxopiperidin-3-yl)-1H-pyrazol-1-yl)piperidine-1-carboxylate 7′ (210 mg, 72.2% yield) as a yellow gummy liquid. It was used for the next step without further purification. LCMS method 1: retention time: 1.684 min, [(M+H)-100]⁺=263.2.

Step 5′. Synthesis of tert-butyl 4-(6-(3-methyl-2,6-dioxopiperidin-3-yl)pyridin-3-yl)piperazine-1-carboxylate 8′. To a stirred solution of tert-butyl 4-(4-(2,6-dioxopiperidin-3-yl)-1H-pyrazol-1-yl)piperidine-1-carboxylate 7′ (0.25 g, 0.545 mmol) in DCM (5 mL) at 0° C. was added 4.0 N HCl in 1,4-dioxane (4.08 mL, 5.45 mmol). The resulting reaction mixture was stirred at room temperature for three h. The reaction was concentrated under reduced pressure to obtain crude 3-(1-(piperidin-4-yl)-1H-pyrazol-4-yl)piperidine-2,6-dione (C-104) (170 mg) as a brown solid. It was used for the next step without further purification. UPLC method 2: retention time: 0.640 min, [M+H]⁺=263.2.

Table 54 summarizes the final compound made from intermediate A87 using general procedures from X-11 and Example S51.

TABLE 54
Final compound made from intermediate A87 using general procedures from X-11 and Example S51

| Compound | Intermediate | |
|---|---|---|
| No. | No. | Structure |
| P-315 | C-12 | 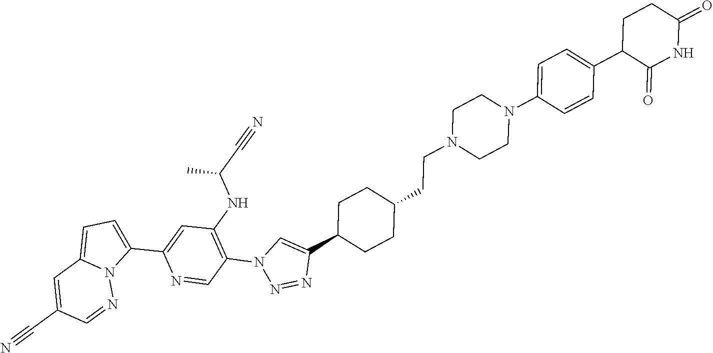 [SMILES: C[C@H](C#N)Nc1cc(-c2ccc3cc(C#N)cnn23)ncc1-n1cc([C@H]2CC[C@H](CCN3CCN(c4ccc(C5CCC(=O)NC5=O)cc4)CC3)CC2)nn1] |

| Compound | |
|---|---|
| No. | Characterization |
| P-315 | 1% yield as yellow solid |
| | (M + H)⁺ = 737.2 ¹H NMR (400 MHz, DMSO-d6): δ |
| | ppm 1.15-1.22 (m, 2H), 1.39-1.55 (m, 3H), 1.66- |
| | 1.68 (m, 5H), 1.86-1.90 (m, 2H), 1.97-2.03 (m, 1H), |
| | 2.14-2.17 (m, 3H), 2.71-2.79 (m, 1H), 2.92-3.00 |
| | (m, 2H), 3.11-3.29 (m, 4H), 3.59-3.62 (m, 2H), 3.73- |
| | 3.79 (m, 1H), 3.81-3.86 (m, 2H), 4.90-4.98 (m, |
| | 1H), 6.97-7.00 (m, 2H), 7.12-7.17 (m, 3H), 7.12- |
| | 7.15 (m, 1H), 7.87 (d, J = 4.8 Hz, 1H), 8.37 (s, 1H), |
| | 8.40 (s, 1H), 8.49 (s, 1H), 8.69 (d, J = 2.4 Hz, 1H), 8.89 |
| | (d, J = 2.4 Hz, 1H), 10.80 (s, 1H) |

Table 55 summarizes Final Compounds Prepared via General Procedure X-8.

TABLE 55
Final Compounds Prepared via General Procedure X-8

| Compound | Intermediate | |
|---|---|---|
| No. | No. | Structure |
| P-275 | C-80 | 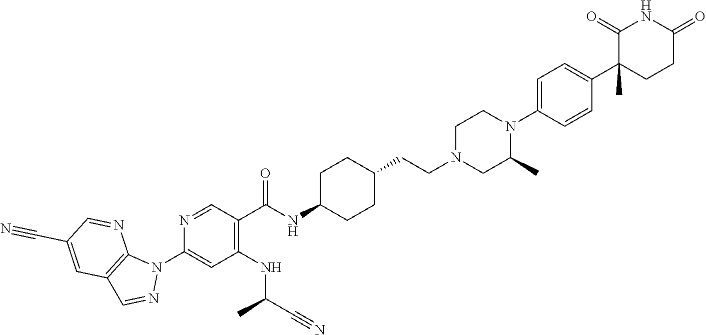 [SMILES: C[C@H](C#N)Nc1cc(-n2ncc3cc(C#N)cnc32)ncc1C(=O)N[C@H]1CC[C@H](CCN2CCN(c3ccc([C@@]4(C)CCC(=O)NC4=O)cc3)[C@@H](C)C2)CC1] |
| P-250 | C-73 | 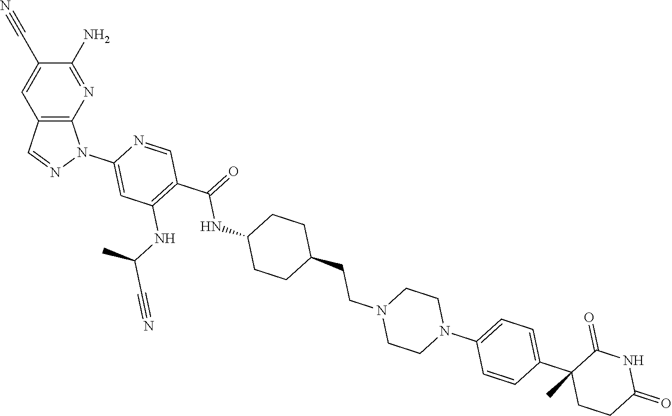 [SMILES: C[C@H](C#N)Nc1cc(-n2ncc3cc(C#N)c(N)nc32)ncc1C(=O)N[C@H]1CC[C@H](CCN2CCN(c3ccc([C@@]4(C)CCC(=O)NC4=O)cc3)CC2)CC1] |
| P-278 | C-97a | 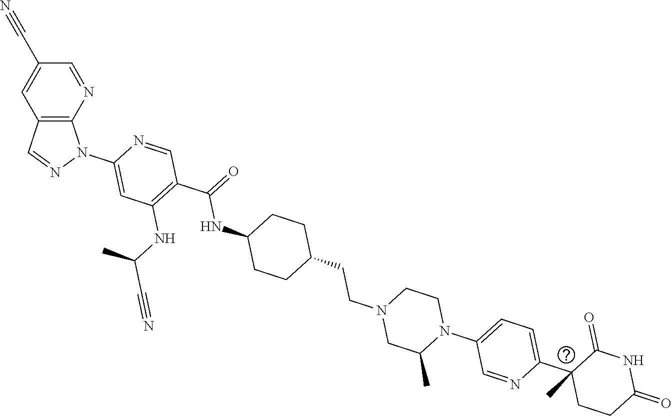 [SMILES: C[C@H](C#N)Nc1cc(-n2ncc3cc(C#N)cnc32)ncc1C(=O)N[C@H]1CC[C@H](CCN2CCN(c3ccc([C@@]4(C)CCC(=O)NC4=O)nc3)[C@@H](C)C2)CC1] |
| P-279 | C-97b | 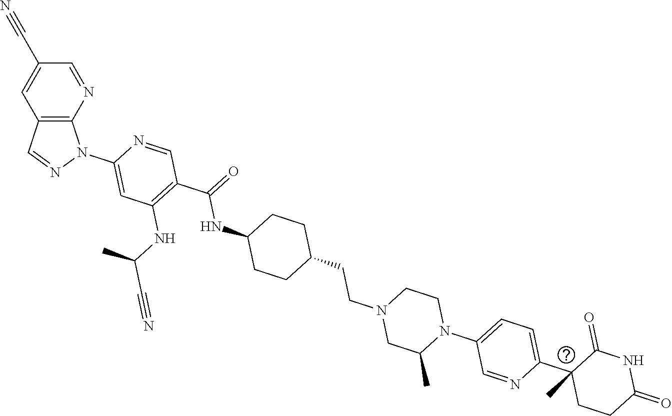 [SMILES: C[C@H](C#N)Nc1cc(-n2ncc3cc(C#N)cnc32)ncc1C(=O)N[C@H]1CC[C@H](CCN2CCN(c3ccc([C@@]4(C)CCC(=O)NC4=O)nc3)[C@@H](C)C2)CC1] |
| P-249 | C-92a | 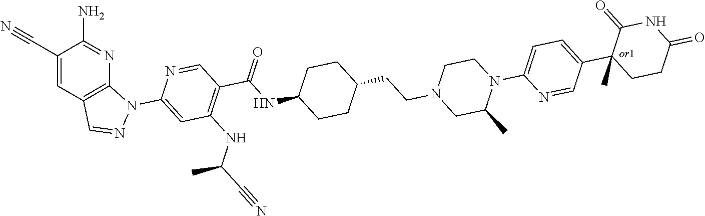 [SMILES: C[C@H](C#N)Nc1cc(-n2ncc3cc(C#N)c(N)nc32)ncc1C(=O)N[C@H]1CC[C@H](CCN2CCN(c3ccc([C@@]4(C)CCC(=O)NC4=O)cn3)[C@@H](C)C2)CC1] |
| P-276 | C-92a | 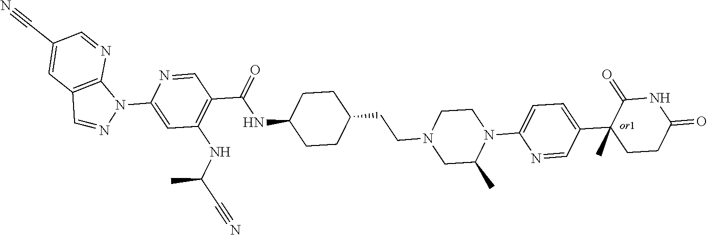 [SMILES: C[C@H](C#N)Nc1cc(-n2ncc3cc(C#N)cnc32)ncc1C(=O)N[C@H]1CC[C@H](CCN2CCN(c3ccc([C@@]4(C)CCC(=O)NC4=O)cn3)[C@@H](C)C2)CC1] |
| P-416 | C-92b | 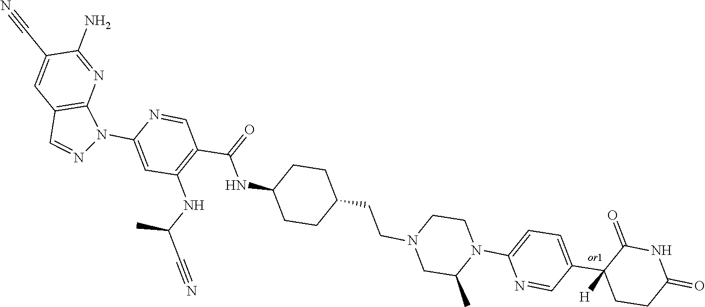 [SMILES: C[C@H](C#N)Nc1cc(-n2ncc3cc(C#N)c(N)nc32)ncc1C(=O)N[C@H]1CC[C@H](CCN2CCN(c3ccc([C@H]4CCC(=O)NC4=O)cn3)[C@@H](C)C2)CC1] |
| P-376 | C-12 | 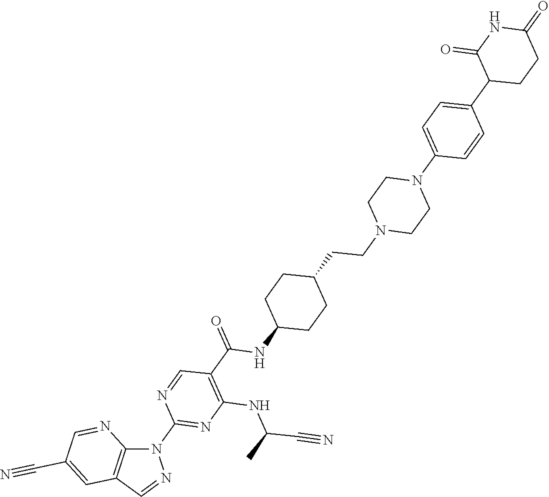 [SMILES: C[C@H](C#N)Nc1nc(-n2ncc3cc(C#N)cnc32)ncc1C(=O)N[C@H]1CC[C@H](CCN2CCN(c3ccc(C4CCC(=O)NC4=O)cc3)CC2)CC1] |
| P-277 | C-92b | 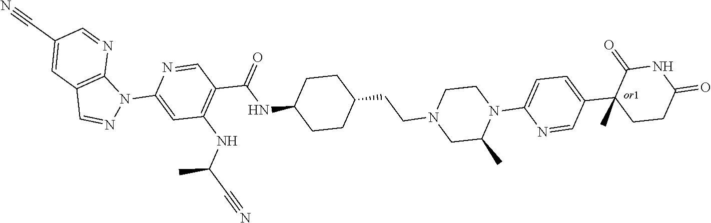 [SMILES: C[C@H](C#N)Nc1cc(-n2ncc3cc(C#N)cnc32)ncc1C(=O)N[C@H]1CC[C@H](CCN2CCN(c3ccc([C@@]4(C)CCC(=O)NC4=O)cn3)[C@@H](C)C2)CC1] |

| Compound | |
|---|---|
| No. | Characterization |
| P-275 | 23% yield as yellow solid |
| | [M + H]⁺ = 742.2 |
| | ¹H NMR (400 MHz, DMSO-d6, 90° C.): δ ppm 1.10- |
| | 1.21 (m, 4H), 1.32-1.44 (m, 6H), 1.64-1.71 (m, 5H), |
| | 1.81-1.84 (m, 2H), 1.91-1.97 (m, 2H), 2.03-2.11 |
| | (m, 2H), 2.42-2.44 (m, 1H), 3.01-3.30 (m, 5H), 3.49- |
| | 3.58 (m, 3H), 3.72-3.77 (m, 1H), 4.33-4.39 (m, |
| | 1H), 4.91-4.96 (m, 1H), 6.94-6.96 (m, 1H), 7.17- |
| | 7.28 (m, 3H), 7.52 (s, 1H), 8.32 (d, J = 7.6 Hz, 1H), |
| | 8.64 (s, 1H), 8.71-8.74 (m, 2H), 8.96 (d, J = 2.0 Hz, |
| | 1H), 8.99 (d, J = 2.0 Hz, 1H), 10.51 (s, 1H) |
| | Two protons are not apparent |
| P-250 | 19% yield off white solid |
| | [M + H]⁺ = 732.4 |
| | ¹H NMR (400 MHz, DMSO-d6): δ ppm 1.01-1.11 (m, |
| | 2H), 1.22-1.35 (m, 3H), 1.40 (s, 3H), 1.58-1.62 (m, |
| | 2H), 1.70 (d, J = 6.8 Hz, 3H), 1.80-1.82 (m, 2H), 1.88- |
| | 1.92 (m, 2H), 2.06-2.08 (m, 1H), 2.41-2.44 (m, |
| | 1H), 2.93-3.00 (m, 2H), 3.09-3.13 (m, 2H), 3.19- |
| | 3.23 (m, 2H), 3.56-3.61 (m, 3H), 3.74-3.80 (m, 1H), |
| | 3.82-3.86 (m, 2H), 4.91-4.99 (m, 1H), 7.01 (d, J = |
| | 9.2 Hz, 2H), 7.18 (d, J = 8.8 Hz, 2H), 7.37 (brs, 2H), |
| | 7.94 (s, 1H), 8.26 (s, 1H), 8.53 (d, J = 7.6 Hz, 1H), 8.58 |
| | (s, 1H), 8.67 (s, 1H), 8.83 (d, J = 6.8 Hz, 1H), 10.87 (s, |
| | 1H). |
| | One proton is not apparent |
| P-278 | 29% yield as off white solid |
| | [M + H]⁺ = 743.4 |
| | ¹H NMR (400 MHz, DMSO-d6, 100° C): δ ppm 1.13- |
| | 1.17 (m, 4H), 1.32-1.46 (m, 3H), 1.53 (s, 3H), 1.66- |
| | 1.71 (m, 5H), 1.84-1.90 (m, 2H), 1.95-2.12 (m, 3H), |
| | 2.21-2.28 (m, 1H), 2.42-2.48 (m, 2H), 3.18-3.35 |
| | (m, 6H), 4.91-4.98 (m, 1H), 7.32-7.34 (m, 1H), 7.42 |
| | (brs, 1H), 7.53 (s, 1H), 8.27-8.29 (m, 2H), 8.63 (s, |
| | 1H), 8.70-8.73 (m, 1H), 8.74 (s, 1H), 8.94 (d, J = 2.0 |
| | Hz, 1H), 8.98 (d, J = 2.0 Hz, 1H), 10.36 (s, 1H) |
| | Five protons are not apparent |
| P-279 | 33% yield as off white solid |
| | [M + H]⁺ = 743.4 |
| | ¹H NMR (400 MHz, DMSO-d6, 100° C.): δ ppm 1.10- |
| | 1.20 (m, 5H), 1.31-1.51 (m, 3H), 1.53 (s, 3H), 1.60- |
| | 1.75 (m, 5H), 1.82-1.91 (m, 2H), 1.96-2.10 (m, 3H), |
| | 2.21-2.33 (m, 1H), 2.40-2.48 (m, 1H), 3.19-3.22 (m, |
| | 2H), 3.25-3.40 (m, 2H), 3.71-3.90 (m, 1H), 4.85- |
| | 4.94 (m, 1H), 7.32-7.34 (m, 1H), 7.44-7.50 (m, 1H), |
| | 7.53 (s, 1H), 8.28-8.30 (m, 2H), 8.63 (s, 1H), 8.71- |
| | 8.75 (m, 2H), 8.94-8.98 (m, 2H), 10.37 (s, 1H). |
| | Six protons are not apparent |
| P-249 | 22% yield as pink solid |
| | [M + H]⁺ = 758.2 |
| | ¹H NMR (400 MHz, DMSO-d6): δ ppm 1.02-1.15 (m, |
| | 2H), 1.21 (d, J = 7.2 Hz, 3H), 1.25-1.42 (m, 4H), 1.43 |
| | (s, 3H), 1.56-1.68 (m, 2H), 1.70 (d, J = 6.8 Hz, 3H), |
| | 179-1.82 (m, 2H), 1.88-1.96 (m, 2H), 2.02-2.18 (m, |
| | 2H), 2.32-2.38 (m, 1H), 2.95-3.21 (m, 5H), 3.72- |
| | 3.81 (m, 2H), 4.32-4.39 (m, 1H), 4.75-3.82 (m, 1H), |
| | 4.92-5.02 (m, 1H), 6.91 (d, J = 9.2 Hz, 1H), 7.40 (brs, |
| | 2H), 7.55-7.58 (m, 1H), 7.98 (s, 1H), 8.03 (d, J = 2.4 |
| | Hz, 1H), 8.27 (s, 1H), 8.55-8.58 (m, 2H), 8.65 (s, 1H), |
| | 8.90-8.92 (d, J = 7.2 Hz, 1H), 10.91 (s, 1H) |
| | One proton is not apparent |
| P-276 | 46% yield as off white solid |
| | [M + H]+ = 743.4 |
| | ¹H NMR (400 MHz, DMSO-d6): δ ppm 1.09-1.12 (m, |
| | 2H), 1.22 (d, J = 6.8 Hz, 3H), 1.25-1.43 (m, 3H), 1.44 |
| | (s, 3H), 1.55-1.64 (m, 2H), 1.68 (d, J = 6.8 Hz, 3H), |
| | 1.83-1.95 (m, 4H), 2.04-2.18 (m, 2H), 2.32-2.40 (m, |
| | 1H), 2.98-3.02 (m, 1H), 3.15-3.26 (m, 4H), 3.52- |
| | 3.61 (m, 2H), 3.75-3.82 (m, 1H), 4.33-4.37 (m, 1H), |
| | 4.78-4.81 (m, 1H), 4.94-4.98 (m, 1H), 6.93 (d, J = 8.8 |
| | Hz, 1H), 7.51 (s, 1H), 7.57-7.60 (m, 1H), 8.04 (d, J = |
| | 2.8 Hz, 1H), 8.57 (d, J = 8.0 Hz, 1H), 8.71 (s, 1H), 8.72 |
| | (s, 1H), 8.78 (d, J = 7.2 Hz, 1H), 9.04-9.06 (m, 2H), |
| | 10.92 (s, 1H). |
| | One proton is not apparent |
| P-416 | 25% yield off white solid |
| | [M + H]⁺ = 758.7 |
| | ¹H NMR (400 MHz, DMSO-d6): δ ppm 1.02-1.18 (m, |
| | 2H), 1.22 (d, J = 6.8 Hz, 3H), 1.27-1.39 (m, 2H), 1.44 |
| | (s, 3H), 1.62-1.66 (m, 2H), 1.71 (d, J = 7.2 Hz, 3H), |
| | 1.76-1.80 (m, 2H), 1.83-1.92 (m, 2H), 2.06-2.14 (m, |
| | 3H), 2.30-2.45 (m, 1H), 2.98-3.01 (m, 1H), 3.14- |
| | 3.21 (m, 5H), 4.34-4.38 (m, 1H), 4.70-4.80 (m, 1H), |
| | 4.95-4.99 (m, 1H), 6.92 (d, J = 8.8 Hz, 1H), 7.38 (brs, |
| | 2H), 7.56-7.59 (m, 1H), 7.97 (s, 1H), 8.04 (d, J = 2.4 |
| | Hz, 1H), 8.27 (s, 1H), 8.55 (d, J = 7.6 Hz, 1H), 8.59 (s, |
| | 1H), 8.66 (s, 1H), 8.88 (d, J = 7.2 Hz, 1H), 10.92 (s, |
| | 1H). |
| | Three protons are not apparent |
| P-376 | 16% yield as pale brown solid |
| | (M + H)⁺ = 715.3 |
| | ¹H NMR (400 MHz, DMSO-d6): δ ppm 1.05-1.14 |
| | (m, 2H), 1.31-1.51 (m, 3H), 1.59-1.64 (m, 2H), 1.72 |
| | (d, J = 7.2 Hz, 3H), 1.82-1.85 (m, 2H), 1.92-.2.01 (m, |
| | 3H), 2.11-2.21 (m, 1H), 2.61-2.70 (m, 1H), 2.92- |
| | 3.00 (m, 2H), 3.11-3.23 (m, 4H), 3.59-3.62 (m, 2H), |
| | 3.73-3.86 (m, 4H), 5.28-5.32 (m, 1H), 7.00 (d, J = |
| | 8.8 Hz, 2H), 7.13 (d, J = 8.8 Hz, 2H), 8.62 (d, J = 7.6 |
| | Hz, 1H), 8.71 (s, 1H), 8.93 (s, 1H), 9.04 (d, J = 2.0 Hz, |
| | 1H), 9.09 (d, J = 2.0 Hz, 1H), 9.27 (d, J = 7.6 Hz, 1H), |
| | 10.79 (s, 1H) |
| | One proton not apparent |
| P-277 | 58% yield as of white solid |
| | [M + H]⁺ = 743.7 |
| | ¹H NMR (400 MHz, DMSO-d6): δ 1.08-1.12 (m, 2H), |
| | 1.21 (d, J = 6.8 Hz, 3H), 1.28-1.30 (m, 3H), 1.43 (s, |
| | 3H), 1.61-1.63 (m, 2H), 1.67 (d, J = 7.2 Hz, 3H), 1.80- |
| | 1.83 (m, 2H), 1.93-1.98 (m, 2H), 2.01-2.20 (m, 2H), |
| | 2.30-2.40 (m, 1H), 2.96-3.04 (m, 1H), 3.14-3.20 (m, |
| | 4H), 4.30-4.40 (m, 1H), 4.70-4.80 (m, 1H), 4.93- |
| | 4.97 (m, 1H), 6.91 (d, J = 8.8 Hz, 1H), 7.49 (s, 1H), |
| | 7.55-7.58 (m, 1H), 8.03 (d, J = 2.4 Hz, 1H), 8.56 (d, J = |
| | 7.6 Hz, 1H), 8.70 (s, 1H), 8.72 (s, 1H), 8.76 (d, J = |
| | 7.6 Hz, 1H), 9.04-9.05 (m, 2H), 10.91 (s, 1H) |
| | Four protons are not apparent |

Table 56 summarizes compounds prepared using General Procedure X-11.

TABLE 56
Final Compounds Prepared via General Procedure X-11

| Compound | Intermediate | |
|---|---|---|
| No. | No. | Structure |
| P-246 | C-73 | 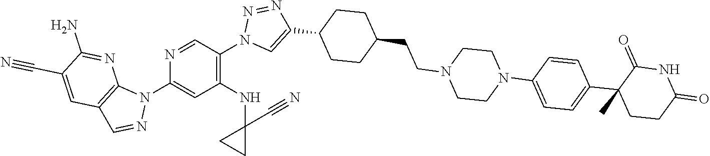 [SMILES: C[C@]1(c2ccc(N3CCN(CC[C@H]4CC[C@H](c5cn(-c6cnc(-n7ncc8cc(C#N)c(N)nc87)cc6NC6(C#N)CC6)nn5)CC4)CC3)cc2)CCC(=O)NC1=O] |
| P-301 | C-79 | 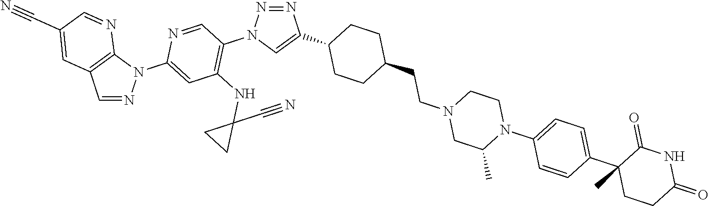 [SMILES: C[C@@H]1CN(CC[C@H]2CC[C@H](c3cn(-c4cnc(-n5ncc6cc(C#N)cnc65)cc4NC4(C#N)CC4)nn3)CC2)CCN1c1ccc([C@@]2(C)CCC(=O)NC2=O)cc1] |
| P-298 | C-73 | 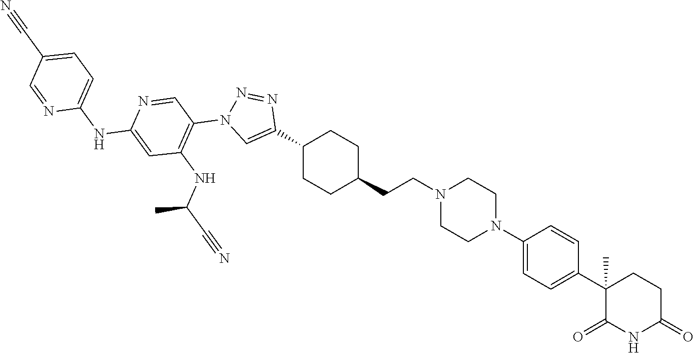 [SMILES: C[C@H](C#N)Nc1cc(Nc2ccc(C#N)cn2)ncc1-n1cc([C@H]2CC[C@H](CCN3CCN(c4ccc([C@@]5(C)CCC(=O)NC5=O)cc4)CC3)CC2)nn1] |
| P-251 | C-81 | 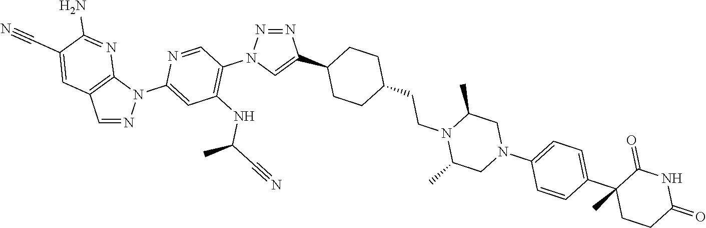 [SMILES: C[C@H](C#N)Nc1cc(-n2ncc3cc(C#N)c(N)nc32)ncc1-n1cc([C@H]2CC[C@H](CCN3[C@@H](C)CN(c4ccc([C@@]5(C)CCC(=O)NC5=O)cc4)C[C@@H]3C)CC2)nn1] |
| P-269 | C-73 | 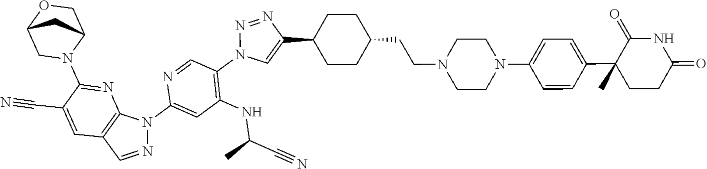 [SMILES: C[C@H](C#N)Nc1cc(-n2ncc3cc(C#N)c(N4C[C@H]5C[C@@H]4CO5)nc32)ncc1-n1cc([C@H]2CC[C@H](CCN3CCN(c4ccc([C@@]5(C)CCC(=O)NC5=O)cc4)CC3)CC2)nn1] |
| P-247 | C-73 | 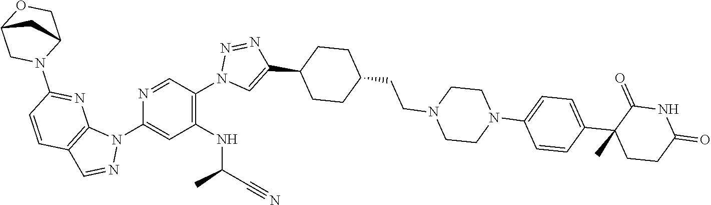 [SMILES: C[C@H](C#N)Nc1cc(-n2ncc3ccc(N4C[C@H]5C[C@@H]4CO5)nc32)ncc1-n1cc([C@H]2CC[C@H](CCN3CCN(c4ccc([C@@]5(C)CCC(=O)NC5=O)cc4)CC3)CC2)nn1] |
| P-297 | C-73 | 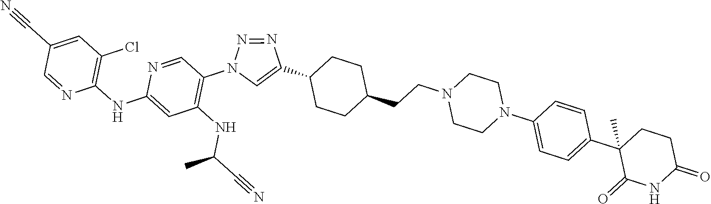 [SMILES: C[C@H](C#N)Nc1cc(Nc2ncc(C#N)cc2Cl)ncc1-n1cc([C@H]2CC[C@H](CCN3CCN(c4ccc([C@@]5(C)CCC(=O)NC5=O)cc4)CC3)CC2)nn1] |
| P-300 | C-73 | 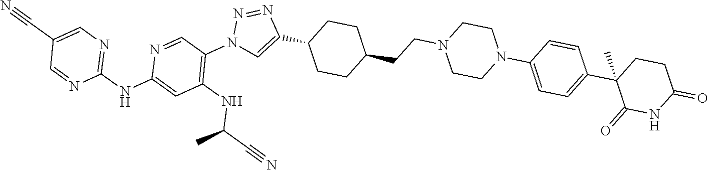 [SMILES: C[C@H](C#N)Nc1cc(Nc2ncc(C#N)cn2)ncc1-n1cc([C@H]2CC[C@H](CCN3CCN(c4ccc([C@@]5(C)CCC(=O)NC5=O)cc4)CC3)CC2)nn1] |
| P-306 | C-103a | 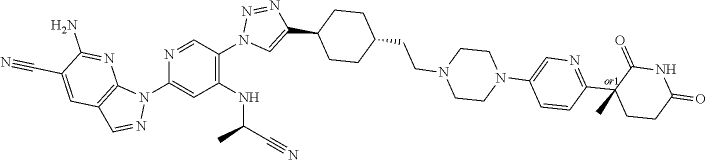 [SMILES: C[C@H](C#N)Nc1cc(-n2ncc3cc(C#N)c(N)nc32)ncc1-n1cc([C@H]2CC[C@H](CCN3CCN(c4ccc([C@@]5(C)CCC(=O)NC5=O)nc4)CC3)CC2)nn1] |
| P-265 | C-98a | 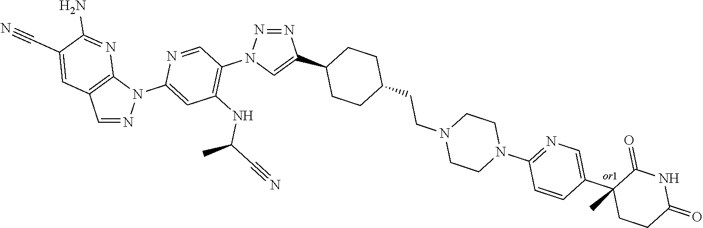 [SMILES: C[C@H](C#N)Nc1cc(-n2ncc3cc(C#N)c(N)nc32)ncc1-n1cc([C@H]2CC[C@H](CCN3CCN(c4ccc([C@@]5(C)CCC(=O)NC5=O)cn4)CC3)CC2)nn1] |
| P-302 | C-101a | 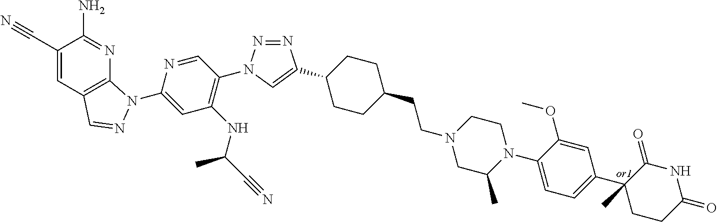 [SMILES: COc1cc([C@@]2(C)CCC(=O)NC2=O)ccc1N1CCN(CC[C@H]2CC[C@H](c3cn(-c4cnc(-n5ncc6cc(C#N)c(N)nc65)cc4N[C@H](C)C#N)nn3)CC2)C[C@@H]1C] |
| P-307 | C-103b | 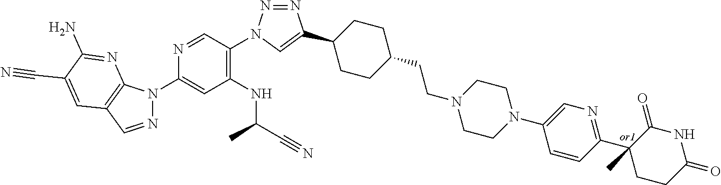 [SMILES: C[C@H](C#N)Nc1cc(-n2ncc3cc(C#N)c(N)nc32)ncc1-n1cc([C@H]2CC[C@H](CCN3CCN(c4ccc([C@@]5(C)CCC(=O)NC5=O)nc4)CC3)CC2)nn1] |
| P-266 | C-94b | 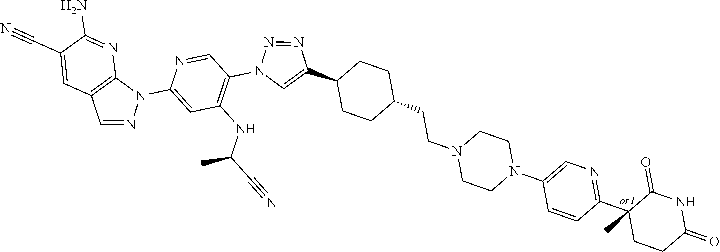 [SMILES: C[C@H](C#N)Nc1cc(-n2ncc3cc(C#N)c(N)nc32)ncc1-n1cc([C@H]2CC[C@H](CCN3CCN(c4ccc([C@@]5(C)CCC(=O)NC5=O)nc4)CC3)CC2)nn1] |
| P-303 | C-101b | 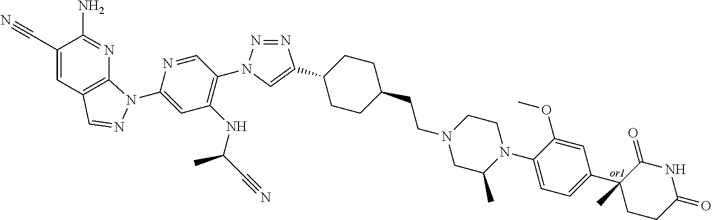 [SMILES: COc1cc([C@@]2(C)CCC(=O)NC2=O)ccc1N1CCN(CC[C@H]2CC[C@H](c3cn(-c4cnc(-n5ncc6cc(C#N)c(N)nc65)cc4N[C@H](C)C#N)nn3)CC2)C[C@@H]1C] |
| P-260 | C-79 | 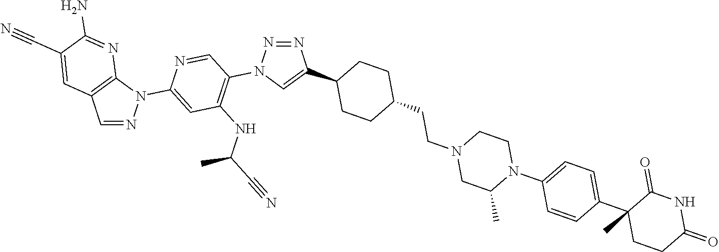 [SMILES: C[C@H](C#N)Nc1cc(-n2ncc3cc(C#N)c(N)nc32)ncc1-n1cc([C@H]2CC[C@H](CCN3CCN(c4ccc([C@@]5(C)CCC(=O)NC5=O)cc4)[C@H](C)C3)CC2)nn1] |
| P-258 | C92a | 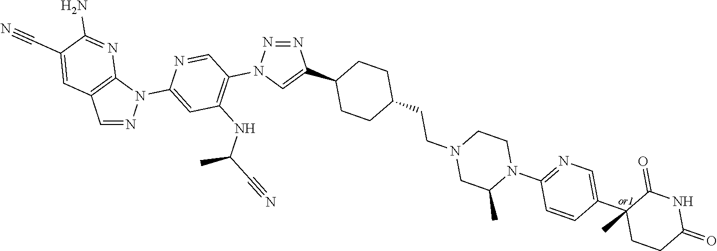 [SMILES: C[C@H](C#N)Nc1cc(-n2ncc3cc(C#N)c(N)nc32)ncc1-n1cc([C@H]2CC[C@H](CCN3CCN(c4ccc([C@@]5(C)CCC(=O)NC5=O)cn4)[C@@H](C)C3)CC2)nn1] |
| P-304 | C-102a | 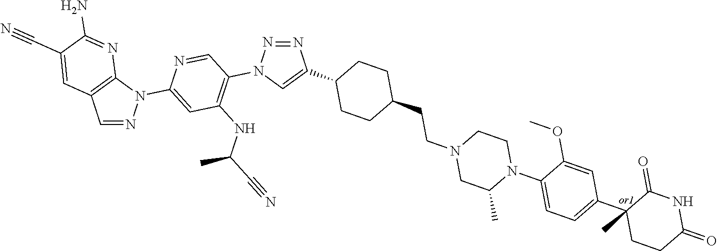 [SMILES: COc1cc([C@@]2(C)CCC(=O)NC2=O)ccc1N1CCN(CC[C@H]2CC[C@H](c3cn(-c4cnc(-n5ncc6cc(C#N)c(N)nc65)cc4N[C@H](C)C#N)nn3)CC2)C[C@H]1C] |
| P-259 | C-92b | 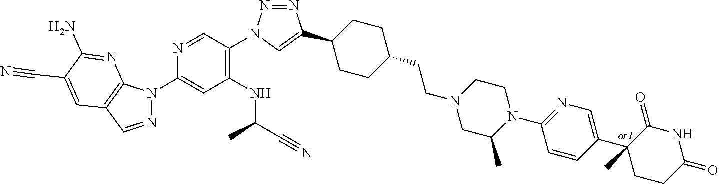 [SMILES: C[C@H](C#N)Nc1cc(-n2ncc3cc(C#N)c(N)nc32)ncc1-n1cc([C@H]2CC[C@H](CCN3CCN(c4ccc([C@@]5(C)CCC(=O)NC5=O)cn4)[C@@H](C)C3)CC2)nn1] |
| P-305 | C-102b | 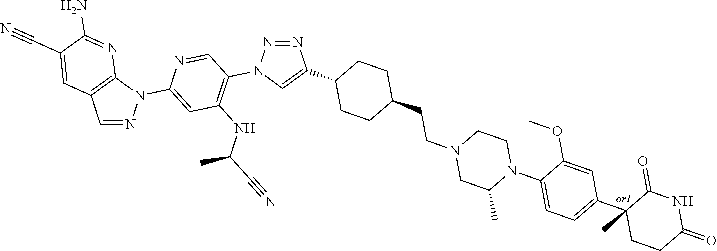 [SMILES: COc1cc([C@@]2(C)CCC(=O)NC2=O)ccc1N1CCN(CC[C@H]2CC[C@H](c3cn(-c4cnc(-n5ncc6cc(C#N)c(N)nc65)cc4N[C@H](C)C#N)nn3)CC2)C[C@H]1C] |
| P-261 | C-98a | 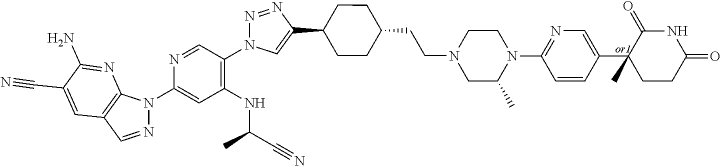 [SMILES: C[C@H](C#N)Nc1cc(-n2ncc3cc(C#N)c(N)nc32)ncc1-n1cc([C@H]2CC[C@H](CCN3CCN(c4ccc([C@@]5(C)CCC(=O)NC5=O)cn4)[C@H](C)C3)CC2)nn1] |
| P-262 | C-98b | 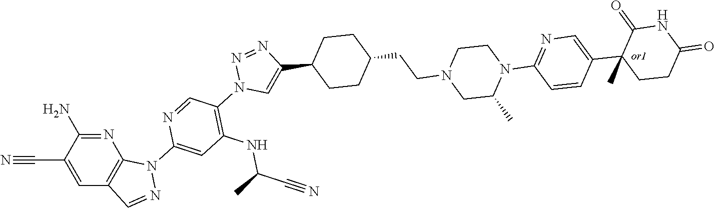 [SMILES: C[C@H](C#N)Nc1cc(-n2ncc3cc(C#N)c(N)nc32)ncc1-n1cc([C@H]2CC[C@H](CCN3CCN(c4ccc([C@@]5(C)CCC(=O)NC5=O)cn4)[C@H](C)C3)CC2)nn1] |
| P-252 | C-95a | 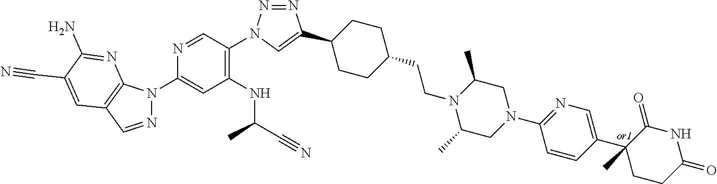 [SMILES: C[C@H](C#N)Nc1cc(-n2ncc3cc(C#N)c(N)nc32)ncc1-n1cc([C@H]2CC[C@H](CCN3[C@@H](C)CN(c4ccc([C@@]5(C)CCC(=O)NC5=O)cn4)C[C@@H]3C)CC2)nn1] |
| P-254 | C-95b | 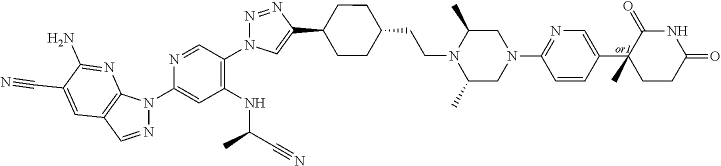 [SMILES: C[C@H](C#N)Nc1cc(-n2ncc3cc(C#N)c(N)nc32)ncc1-n1cc([C@H]2CC[C@H](CCN3[C@@H](C)CN(c4ccc([C@@]5(C)CCC(=O)NC5=O)cn4)C[C@@H]3C)CC2)nn1] |
| P-253 | C-96a | 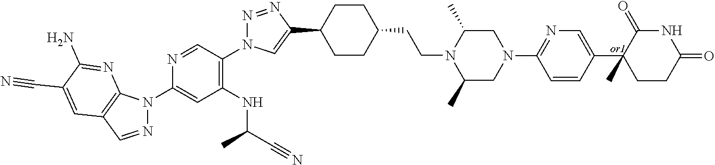 [SMILES: C[C@H](C#N)Nc1cc(-n2ncc3cc(C#N)c(N)nc32)ncc1-n1cc([C@H]2CC[C@H](CCN3[C@H](C)CN(c4ccc([C@@]5(C)CCC(=O)NC5=O)cn4)C[C@H]3C)CC2)nn1] |
| P-255 | C-96b | 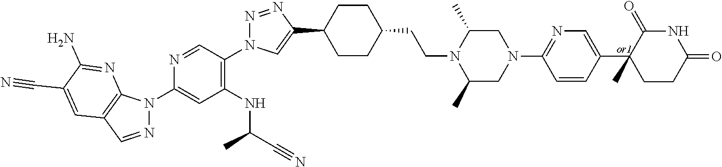 [SMILES: C[C@H](C#N)Nc1cc(-n2ncc3cc(C#N)c(N)nc32)ncc1-n1cc([C@H]2CC[C@H](CCN3[C@H](C)CN(c4ccc([C@@]5(C)CCC(=O)NC5=O)cn4)C[C@H]3C)CC2)nn1] |
| P-263 | C-99a | 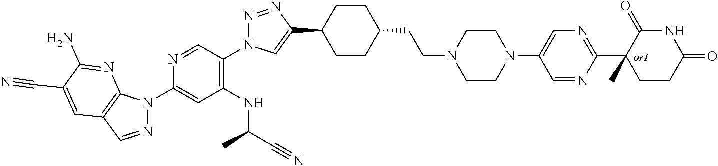 [SMILES: C[C@H](C#N)Nc1cc(-n2ncc3cc(C#N)c(N)nc32)ncc1-n1cc([C@H]2CC[C@H](CCN3CCN(c4cnc([C@@]5(C)CCC(=O)NC5=O)nc4)CC3)CC2)nn1] |
| P-264 | C-99b | 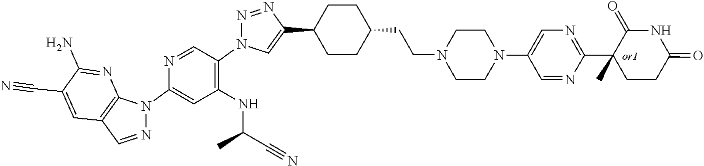 [SMILES: C[C@H](C#N)Nc1cc(-n2ncc3cc(C#N)c(N)nc32)ncc1-n1cc([C@H]2CC[C@H](CCN3CCN(c4cnc([C@@]5(C)CCC(=O)NC5=O)nc4)CC3)CC2)nn1] |
| P-308 | C-102a | 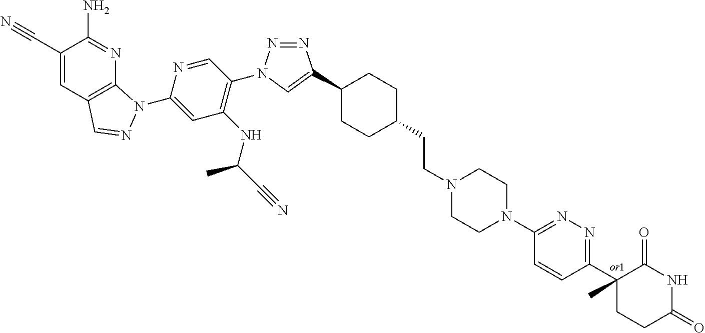 [SMILES: C[C@H](C#N)Nc1cc(-n2ncc3cc(C#N)c(N)nc32)ncc1-n1cc([C@H]2CC[C@H](CCN3CCN(c4ccc([C@@]5(C)CCC(=O)NC5=O)nn4)CC3)CC2)nn1] |
| P-309 | C-102b | 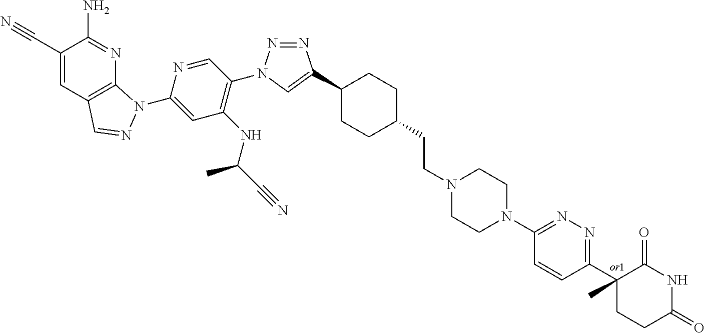 [SMILES: C[C@H](C#N)Nc1cc(-n2ncc3cc(C#N)c(N)nc32)ncc1-n1cc([C@H]2CC[C@H](CCN3CCN(c4ccc([C@@]5(C)CCC(=O)NC5=O)nn4)CC3)CC2)nn1] |
| P-294 | C-103b | 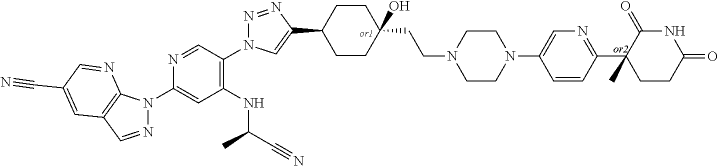 [SMILES: C[C@H](C#N)Nc1cc(-n2ncc3cc(C#N)cnc32)ncc1-n1cc([C@H]2CC[C@](O)(CCN3CCN(c4ccc([C@@]5(C)CCC(=O)NC5=O)nc4)CC3)CC2)nn1] |
| P-296 | C-94a | 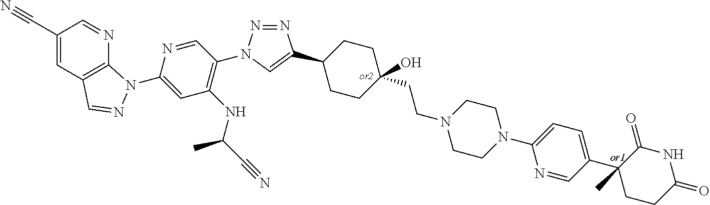 [SMILES: C[C@H](C#N)Nc1cc(-n2ncc3cc(C#N)cnc32)ncc1-n1cc([C@H]2CC[C@](O)(CCN3CCN(c4ccc([C@@]5(C)CCC(=O)NC5=O)cn4)CC3)CC2)nn1] |
| P-267 | C-100a | 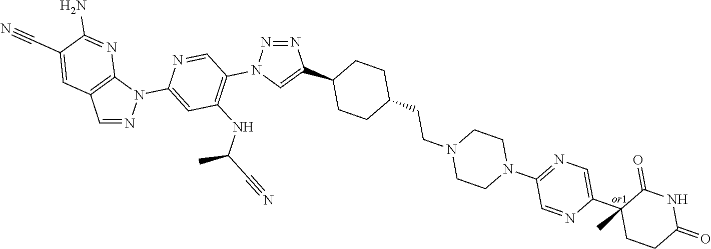 [SMILES: C[C@H](C#N)Nc1cc(-n2ncc3cc(C#N)c(N)nc32)ncc1-n1cc([C@H]2CC[C@H](CCN3CCN(c4cnc([C@@]5(C)CCC(=O)NC5=O)cn4)CC3)CC2)nn1] |
| P-268 | C-100b | 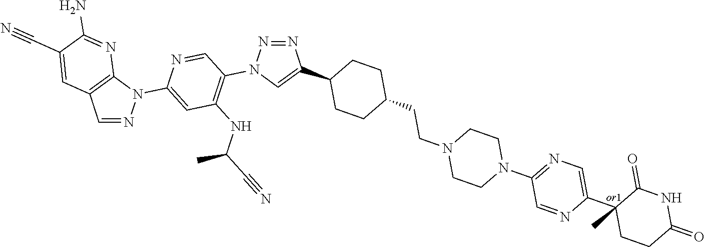 [SMILES: C[C@H](C#N)Nc1cc(-n2ncc3cc(C#N)c(N)nc32)ncc1-n1cc([C@H]2CC[C@H](CCN3CCN(c4cnc([C@@]5(C)CCC(=O)NC5=O)cn4)CC3)CC2)nn1] |
| P-293 | C-98b | 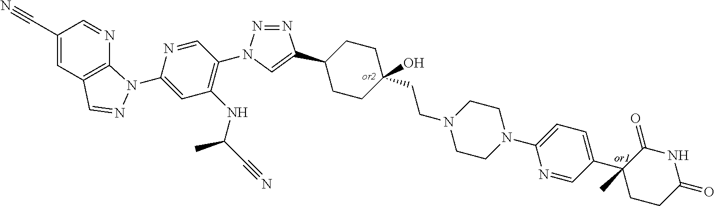 [SMILES: C[C@H](C#N)Nc1cc(-n2ncc3cc(C#N)cnc32)ncc1-n1cc([C@H]2CC[C@](O)(CCN3CCN(c4ccc([C@@]5(C)CCC(=O)NC5=O)cn4)CC3)CC2)nn1] |
| P-280 | C-103a | 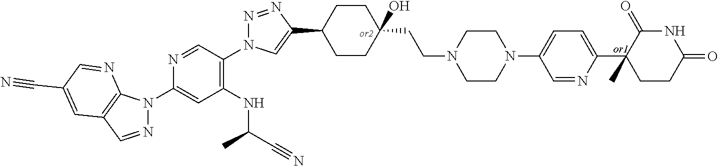 [SMILES: C[C@H](C#N)Nc1cc(-n2ncc3cc(C#N)cnc32)ncc1-n1cc([C@H]2CC[C@](O)(CCN3CCN(c4ccc([C@@]5(C)CCC(=O)NC5=O)nc4)CC3)CC2)nn1] |
| P-295 | C-103b | 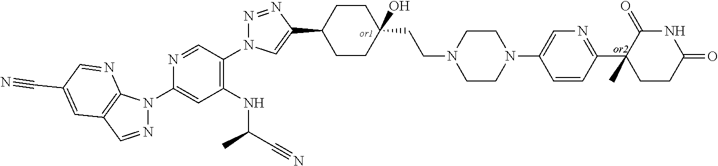 [SMILES: C[C@H](C#N)Nc1cc(-n2ncc3cc(C#N)cnc32)ncc1-n1cc([C@H]2CC[C@](O)(CCN3CCN(c4ccc([C@@]5(C)CCC(=O)NC5=O)nc4)CC3)CC2)nn1] |
| P-256 | C-97a | 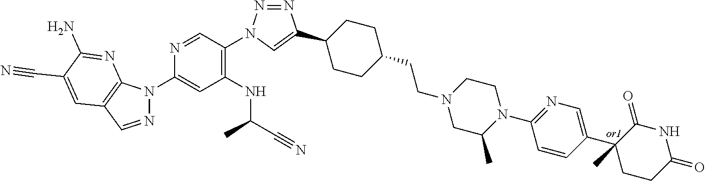 [SMILES: C[C@H](C#N)Nc1cc(-n2ncc3cc(C#N)c(N)nc32)ncc1-n1cc([C@H]2CC[C@H](CCN3CCN(c4ccc([C@@]5(C)CCC(=O)NC5=O)cn4)[C@@H](C)C3)CC2)nn1] |
| P-312 | C-93a | 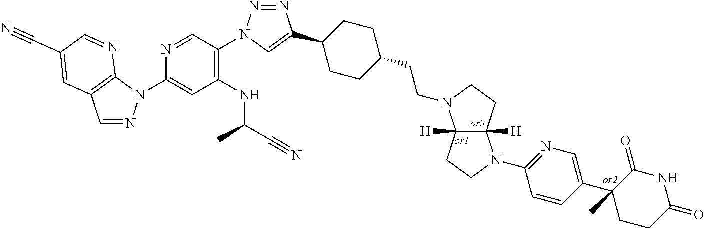 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc([C@@]4(C)CCC(=O)NC4=O)cn3)CC2)nn1] |
| P-289 | C-97a | 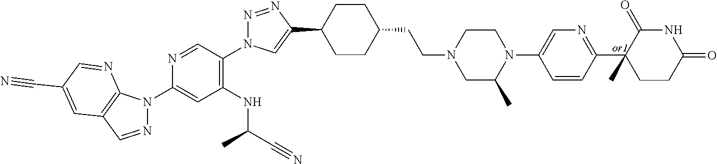 [SMILES: C[C@H](C#N)Nc1cc(-n2ncc3cc(C#N)cnc32)ncc1-n1cc([C@H]2CC[C@H](CCN3CCN(c4ccc([C@@]5(C)CCC(=O)NC5=O)nc4)[C@@H](C)C3)CC2)nn1] |
| P-257 | C-97b | 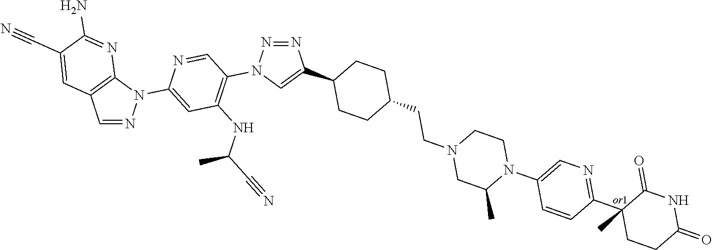 [SMILES: C[C@H](C#N)Nc1cc(-n2ncc3cc(C#N)c(N)nc32)ncc1-n1cc([C@H]2CC[C@H](CCN3CCN(c4ccc([C@@]5(C)CCC(=O)NC5=O)nc4)[C@@H](C)C3)CC2)nn1] |
| P-290 | C-97b | 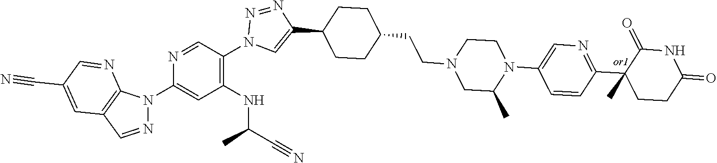 [SMILES: C[C@H](C#N)Nc1cc(-n2ncc3cc(C#N)cnc32)ncc1-n1cc([C@H]2CC[C@H](CCN3CCN(c4ccc([C@@]5(C)CCC(=O)NC5=O)nc4)[C@@H](C)C3)CC2)nn1] |
| P-286 | C-93c | 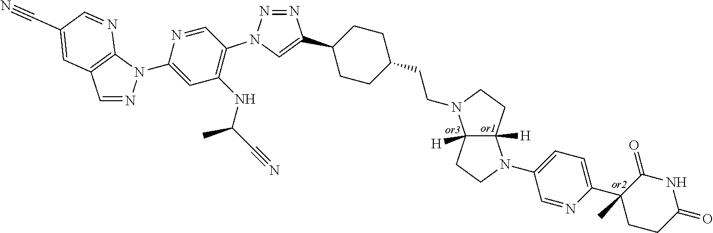 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc([C@@]4(C)CCC(=O)NC4=O)nc3)CC2)nn1] |
| P-287 | C-93d | 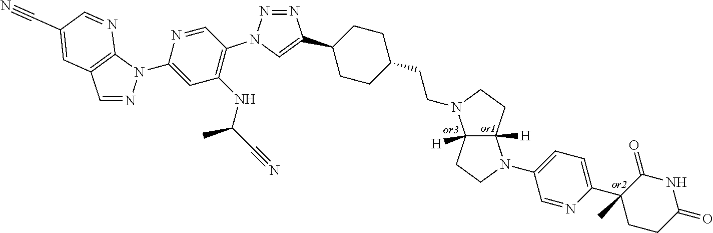 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc([C@@]4(C)CCC(=O)NC4=O)nc3)CC2)nn1] |
| P-313 | C-93b | 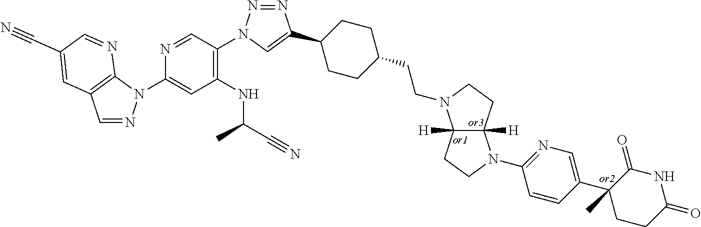 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc([C@@]4(C)CCC(=O)NC4=O)cn3)CC2)nn1] |
| P-405 | C-12 | 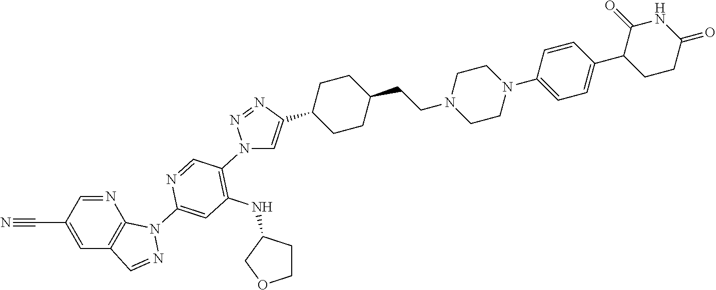 [SMILES: N#Cc1cnc2c(cnn2-c2cc(N[C@@H]3CCOC3)c(-n3cc([C@H]4CC[C@H](CCN5CCN(c6ccc(C7CCC(=O)NC7=O)cc6)CC5)CC4)nn3)cn2)c1] |
| P-409 | C-12 | 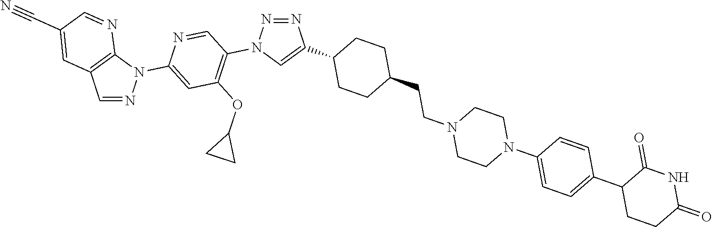 [SMILES: N#Cc1cnc2c(cnn2-c2cc(OC3CC3)c(-n3cc([C@H]4CC[C@H](CCN5CCN(c6ccc(C7CCC(=O)NC7=O)cc6)CC5)CC4)nn3)cn2)c1] |
| P-406 | C-12 | 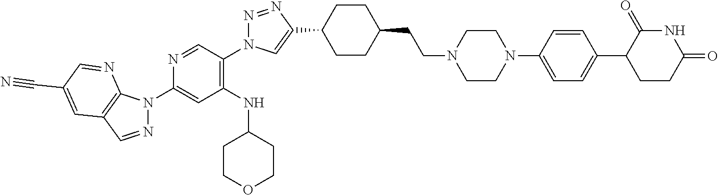 [SMILES: N#Cc1cnc2c(cnn2-c2cc(NC3CCOCC3)c(-n3cc([C@H]4CC[C@H](CCN5CCN(c6ccc(C7CCC(=O)NC7=O)cc6)CC5)CC4)nn3)cn2)c1] |
| P-408 | C-12 | 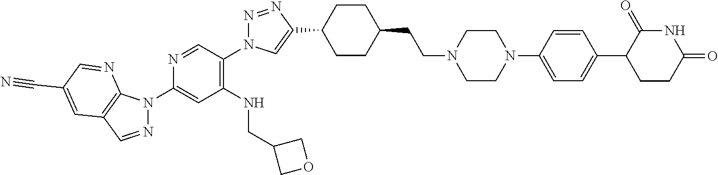 [SMILES: N#Cc1cnc2c(cnn2-c2cc(NCC3COC3)c(-n3cc([C@H]4CC[C@H](CCN5CCN(c6ccc(C7CCC(=O)NC7=O)cc6)CC5)CC4)nn3)cn2)c1] |
| P-395 | C-12 | 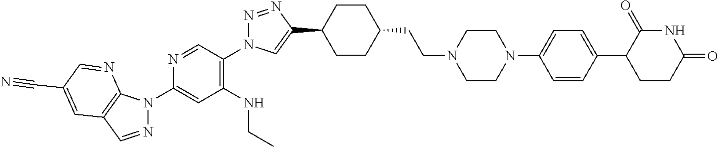 [SMILES: CCNc1cc(-n2ncc3cc(C#N)cnc32)ncc1-n1cc([C@H]2CC[C@H](CCN3CCN(c4ccc(C5CCC(=O)NC5=O)cc4)CC3)CC2)nn1] |
| P-299 | C-73 | 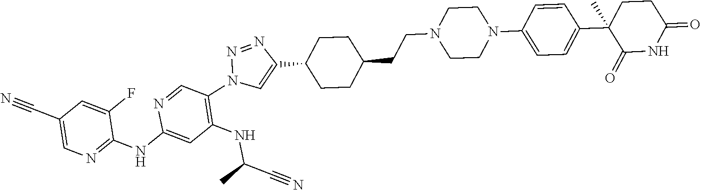 [SMILES: C[C@H](C#N)Nc1cc(Nc2ncc(C#N)cc2F)ncc1-n1cc([C@H]2CC[C@H](CCN3CCN(c4ccc([C@@]5(C)CCC(=O)NC5=O)cc4)CC3)CC2)nn1] |
| P-377 | C-12 | 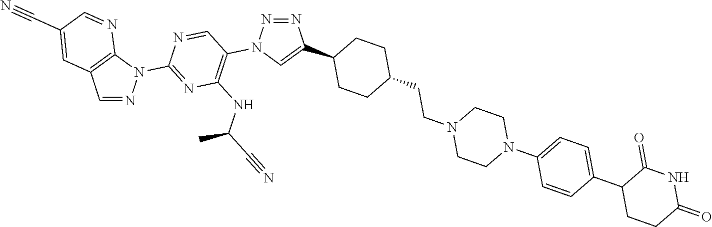 [SMILES: C[C@H](C#N)Nc1nc(-n2ncc3cc(C#N)cnc32)ncc1-n1cc([C@H]2CC[C@H](CCN3CCN(c4ccc(C5CCC(=O)NC5=O)cc4)CC3)CC2)nn1] |
| P-392 | C-12 | 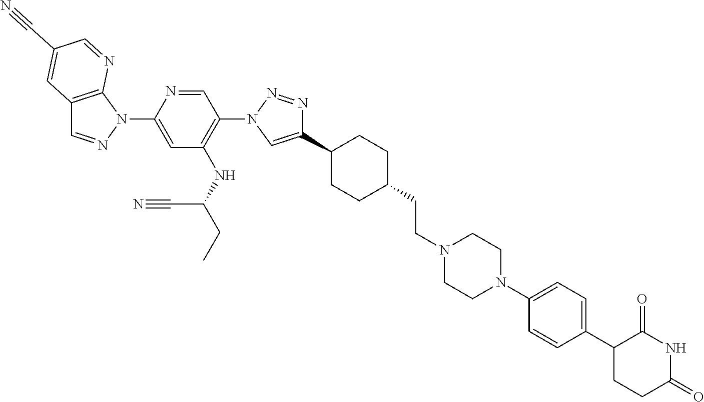 [SMILES: CC[C@H](C#N)Nc1cc(-n2ncc3cc(C#N)cnc32)ncc1-n1cc([C@H]2CC[C@H](CCN3CCN(c4ccc(C5CCC(=O)NC5=O)cc4)CC3)CC2)nn1] |
| P-402 | C-12 | 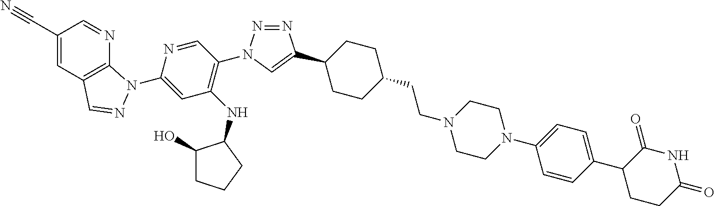 [SMILES: N#Cc1cnc2c(cnn2-c2cc(N[C@H]3CCC[C@H]3O)c(-n3cc([C@H]4CC[C@H](CCN5CCN(c6ccc(C7CCC(=O)NC7=O)cc6)CC5)CC4)nn3)cn2)c1] |
| P-397 | C-12 | 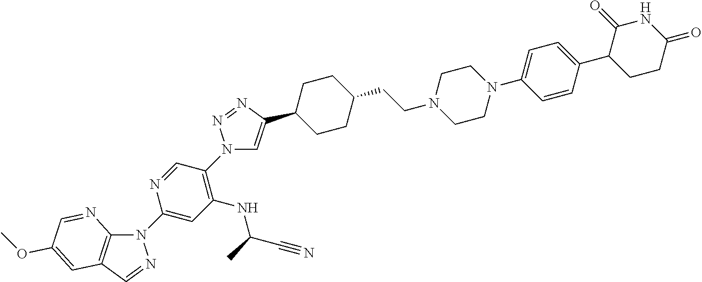 [SMILES: COc1cnc2c(cnn2-c2cc(N[C@H](C)C#N)c(-n3cc([C@H]4CC[C@H](CCN5CCN(c6ccc(C7CCC(=O)NC7=O)cc6)CC5)CC4)nn3)cn2)c1] |
| P-403 | C-12 | 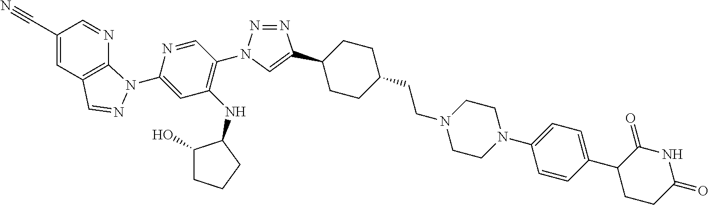 [SMILES: N#Cc1cnc2c(cnn2-c2cc(N[C@H]3CCC[C@@H]3O)c(-n3cc([C@H]4CC[C@H](CCN5CCN(c6ccc(C7CCC(=O)NC7=O)cc6)CC5)CC4)nn3)cn2)c1] |
| P-410 | C-12 | 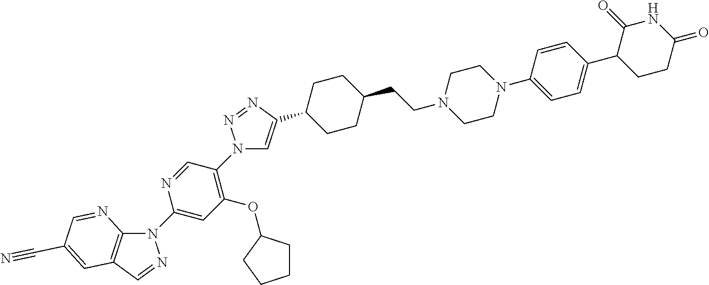 [SMILES: N#Cc1cnc2c(cnn2-c2cc(OC3CCCC3)c(-n3cc([C@H]4CC[C@H](CCN5CCN(c6ccc(C7CCC(=O)NC7=O)cc6)CC5)CC4)nn3)cn2)c1] |
| P-411 | C-12 | 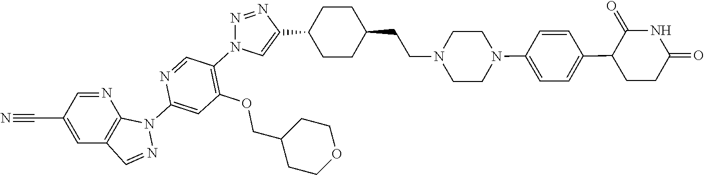 [SMILES: N#Cc1cnc2c(cnn2-c2cc(OCC3CCOCC3)c(-n3cc([C@H]4CC[C@H](CCN5CCN(c6ccc(C7CCC(=O)NC7=O)cc6)CC5)CC4)nn3)cn2)c1] |
| P-375 | C-12 | 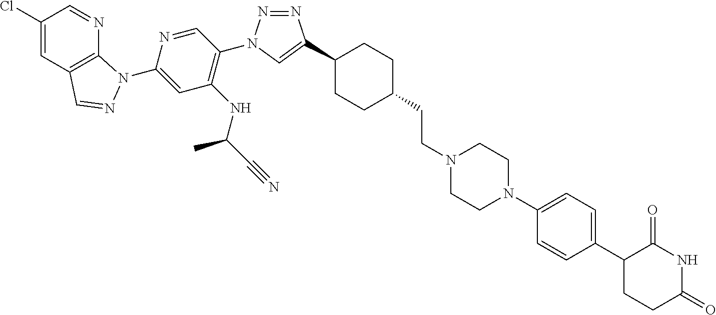 [SMILES: C[C@H](C#N)Nc1cc(-n2ncc3cc(Cl)cnc32)ncc1-n1cc([C@H]2CC[C@H](CCN3CCN(c4ccc(C5CCC(=O)NC5=O)cc4)CC3)CC2)nn1] |
| P-391 | C-12 | 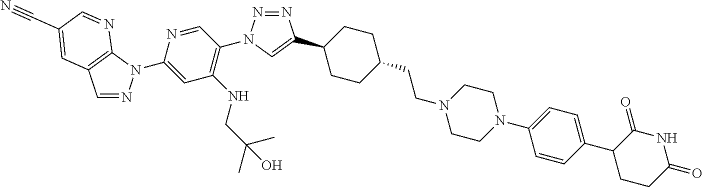 [SMILES: CC(C)(O)CNc1cc(-n2ncc3cc(C#N)cnc32)ncc1-n1cc([C@H]2CC[C@H](CCN3CCN(c4ccc(C5CCC(=O)NC5=O)cc4)CC3)CC2)nn1] |
| P-380 | C-12 | 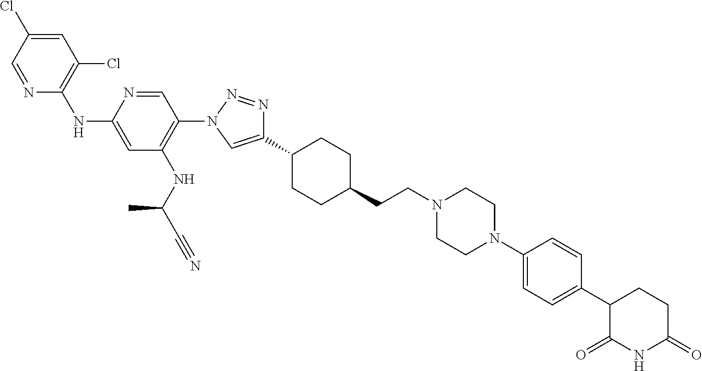 [SMILES: C[C@H](C#N)Nc1cc(Nc2ncc(Cl)cc2Cl)ncc1-n1cc([C@H]2CC[C@H](CCN3CCN(c4ccc(C5CCC(=O)NC5=O)cc4)CC3)CC2)nn1] |
| P-388 | C-12 | 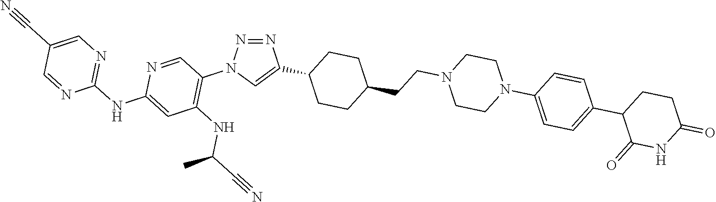 [SMILES: C[C@H](C#N)Nc1cc(Nc2ncc(C#N)cn2)ncc1-n1cc([C@H]2CC[C@H](CCN3CCN(c4ccc(C5CCC(=O)NC5=O)cc4)CC3)CC2)nn1] |
| P-383 | C-12 | 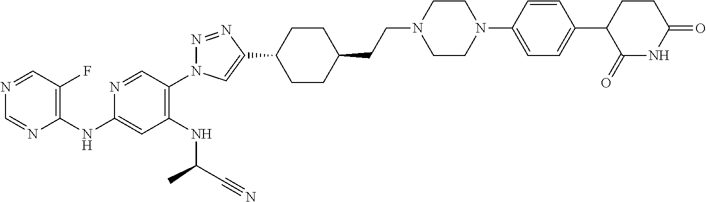 [SMILES: C[C@H](C#N)Nc1cc(Nc2ncncc2F)ncc1-n1cc([C@H]2CC[C@H](CCN3CCN(c4ccc(C5CCC(=O)NC5=O)cc4)CC3)CC2)nn1] |
| P-398 | C-12 | 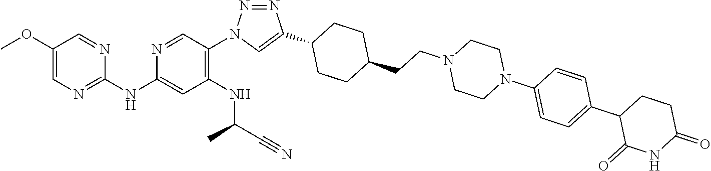 [SMILES: COc1cnc(Nc2cc(N[C@H](C)C#N)c(-n3cc([C@H]4CC[C@H](CCN5CCN(c6ccc(C7CCC(=O)NC7=O)cc6)CC5)CC4)nn3)cn2)nc1] |
| P-382 | C-12 | 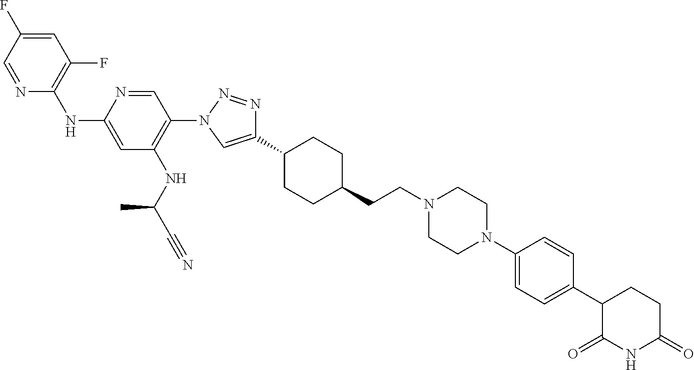 [SMILES: C[C@H](C#N)Nc1cc(Nc2ncc(F)cc2F)ncc1-n1cc([C@H]2CC[C@H](CCN3CCN(c4ccc(C5CCC(=O)NC5=O)cc4)CC3)CC2)nn1] |
| P-381 | C-12 | 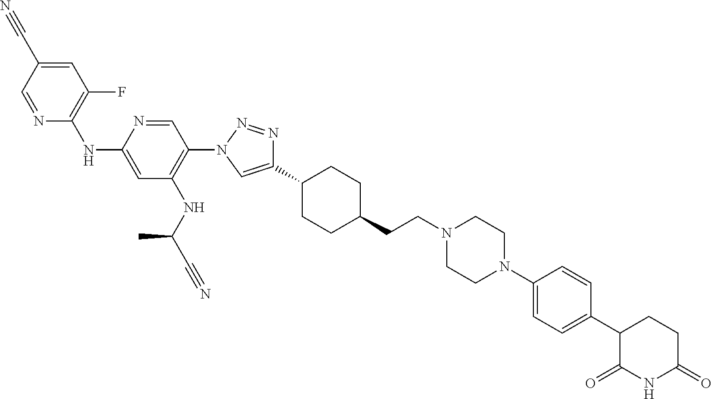 [SMILES: C[C@H](C#N)Nc1cc(Nc2ncc(C#N)cc2F)ncc1-n1cc([C@H]2CC[C@H](CCN3CCN(c4ccc(C5CCC(=O)NC5=O)cc4)CC3)CC2)nn1] |
| P-379 | C-12 | 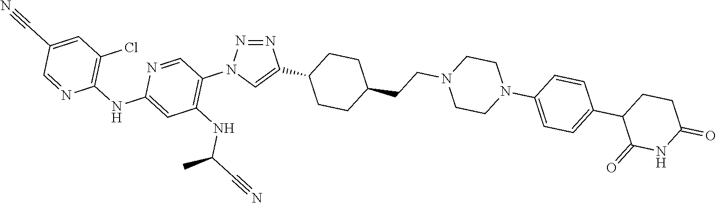 [SMILES: C[C@H](C#N)Nc1cc(Nc2ncc(C#N)cc2Cl)ncc1-n1cc([C@H]2CC[C@H](CCN3CCN(c4ccc(C5CCC(=O)NC5=O)cc4)CC3)CC2)nn1] |
| P-415 | C-91b | 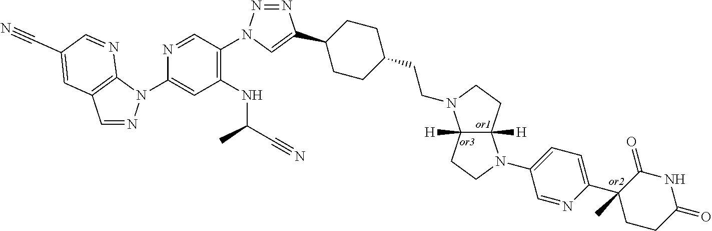 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc([C@@]4(C)CCC(=O)NC4=O)nc3)CC2)nn1] |
| P-418 | C-91c | 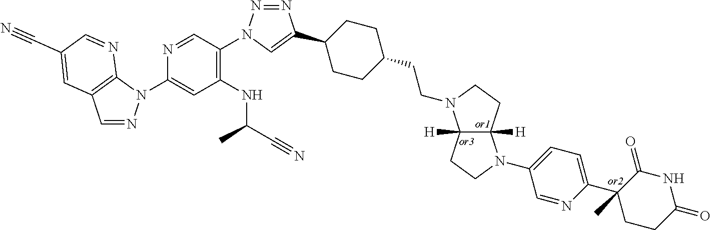 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc([C@@]4(C)CCC(=O)NC4=O)nc3)CC2)nn1] |
| P-419 | C-103a | 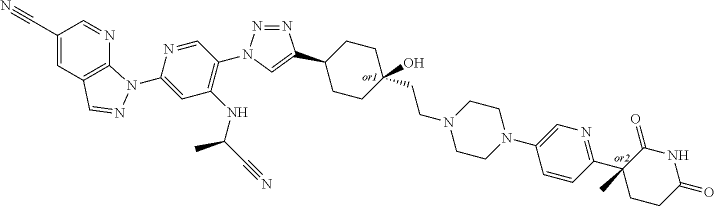 [SMILES: C[C@H](C#N)Nc1cc(-n2ncc3cc(C#N)cnc32)ncc1-n1cc([C@H]2CC[C@](O)(CCN3CCN(c4ccc([C@@]5(C)CCC(=O)NC5=O)nc4)CC3)CC2)nn1] |
| P-426 | C-94a | 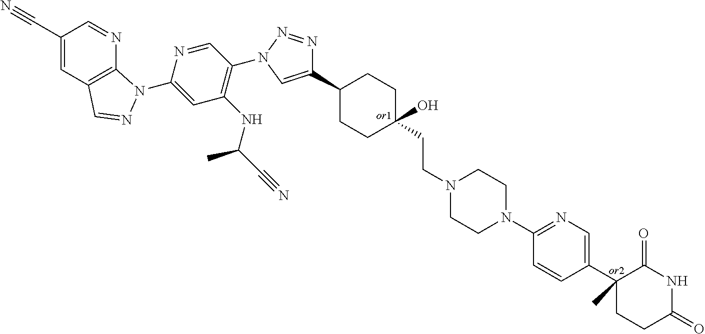 [SMILES: C[C@H](C#N)Nc1cc(-n2ncc3cc(C#N)cnc32)ncc1-n1cc([C@H]2CC[C@](O)(CCN3CCN(c4ccc([C@@]5(C)CCC(=O)NC5=O)cn4)CC3)CC2)nn1] |
| P-427 | C-88b | 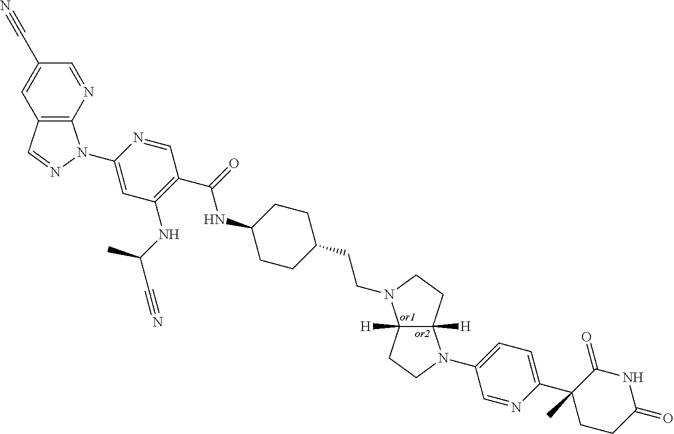 [SMILES: C[C@H](C#N)Nc1cc(-n2ncc3cc(C#N)cnc32)ncc1C(=O)N[C@H]1CC[C@H](CCN2CC[C@H]3[C@@H]2CCN3c2ccc([C@@]3(C)CCC(=O)NC3=O)nc2)CC1] |
| P-435 | C-93d | 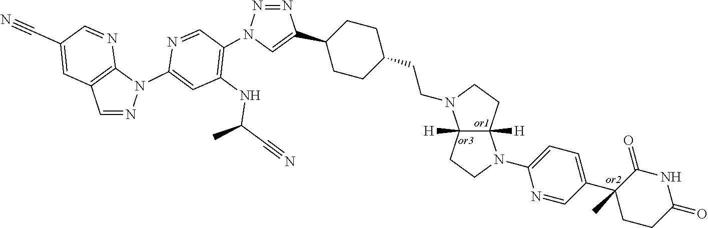 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc([C@@]4(C)CCC(=O)NC4=O)cn3)CC2)nn1] |
| P-292 | C-73 | 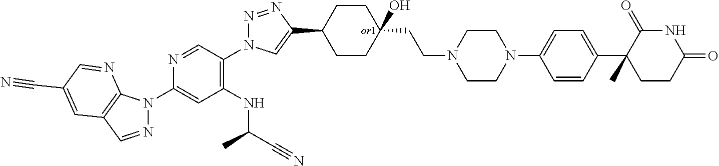 [SMILES: C[C@H](C#N)Nc1cc(-n2ncc3cc(C#N)cnc32)ncc1-n1cc([C@H]2CC[C@](O)(CCN3CCN(c4ccc([C@@]5(C)CCC(=O)NC5=O)cc4)CC3)CC2)nn1] |
| P-288 | C-91a | 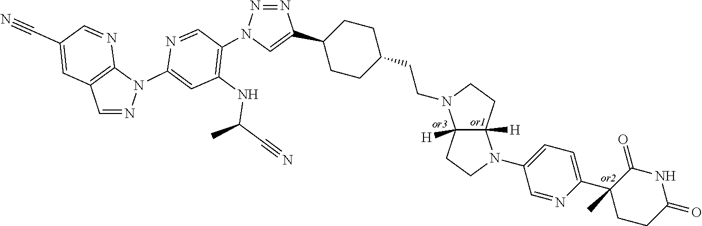 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc([C@@]4(C)CCC(=O)NC4=O)nc3)CC2)nn1] |
| P-291 | C-80 | 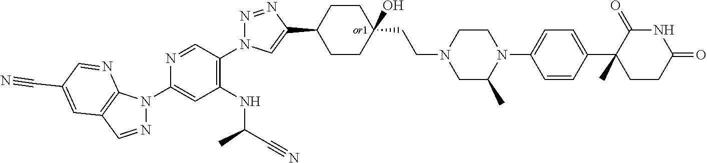 [SMILES: C[C@H](C#N)Nc1cc(-n2ncc3cc(C#N)cnc32)ncc1-n1cc([C@H]2CC[C@](O)(CCN3CCN(c4ccc([C@@]5(C)CCC(=O)NC5=O)cc4)[C@@H](C)C3)CC2)nn1] |
| P-346 | C-104 | 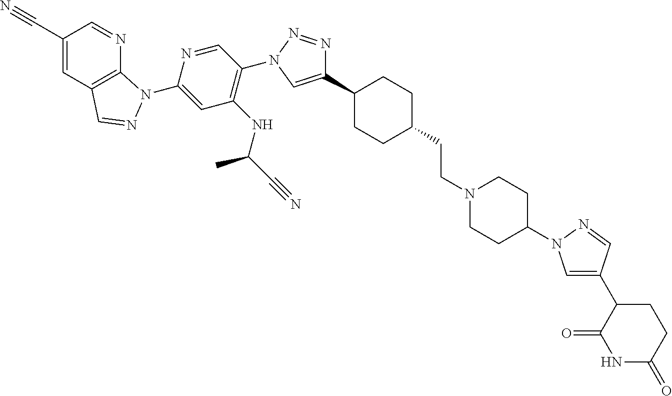 [SMILES: C[C@H](C#N)Nc1cc(-n2ncc3cc(C#N)cnc32)ncc1-n1cc([C@H]2CC[C@H](CCN3CCC(n4cc(C5CCC(=O)NC5=O)cn4)CC3)CC2)nn1] |
| P-442 | C-85 | 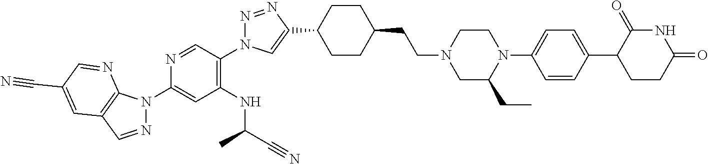 [SMILES: CC[C@H]1CN(CC[C@H]2CC[C@H](c3cn(-c4cnc(-n5ncc6cc(C#N)cnc65)cc4N[C@H](C)C#N)nn3)CC2)CCN1c1ccc(C2CCC(=O)NC2=O)cc1] |
| P-448 | C-89 | 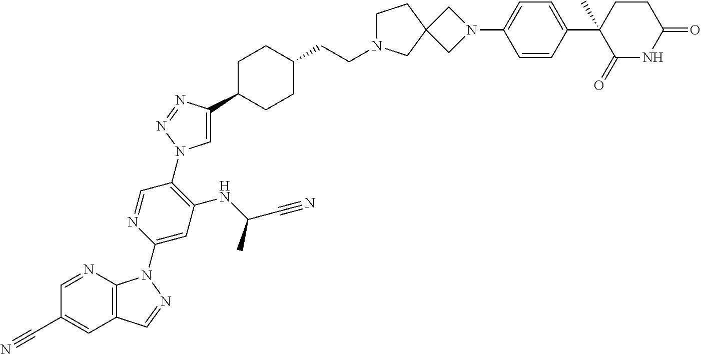 [SMILES: C[C@H](C#N)Nc1cc(-n2ncc3cc(C#N)cnc32)ncc1-n1cc([C@H]2CC[C@H](CCN3CCC4(C3)CN(c3ccc([C@@]5(C)CCC(=O)NC5=O)cc3)C4)CC2)nn1] |
| P-452 | C-86 | 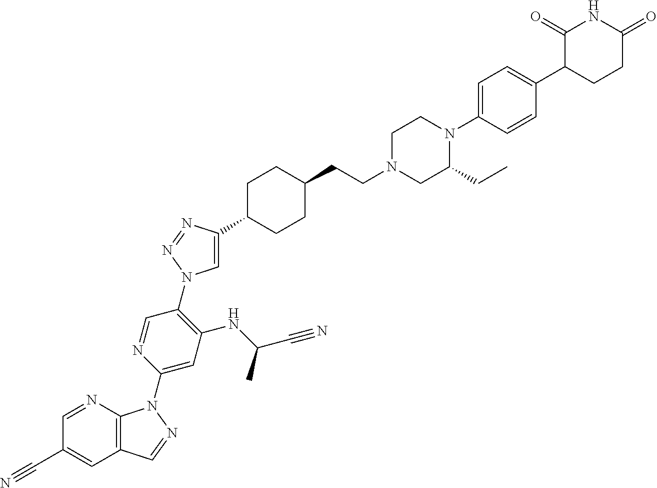 [SMILES: CC[C@@H]1CN(CC[C@H]2CC[C@H](c3cn(-c4cnc(-n5ncc6cc(C#N)cnc65)cc4N[C@H](C)C#N)nn3)CC2)CCN1c1ccc(C2CCC(=O)NC2=O)cc1] |
| P-453 | C-88a | 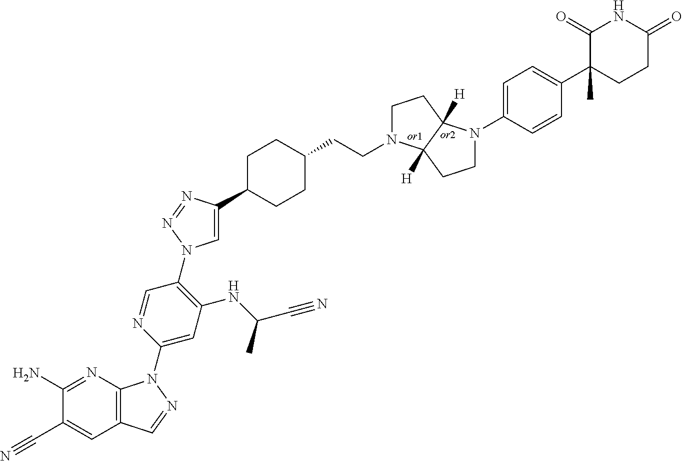 [SMILES: C[C@H](C#N)Nc1cc(-n2ncc3cc(C#N)c(N)nc32)ncc1-n1cc([C@H]2CC[C@H](CCN3CC[C@H]4[C@@H]3CCN4c3ccc([C@@]4(C)CCC(=O)NC4=O)cc3)CC2)nn1] |
| P-454 | C-88a | 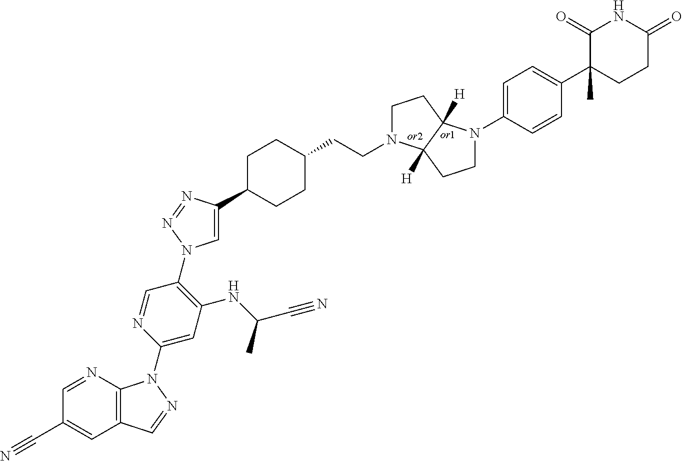 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc([C@@]4(C)CCC(=O)NC4=O)cc3)CC2)nn1] |
| P-462 | C-88b | 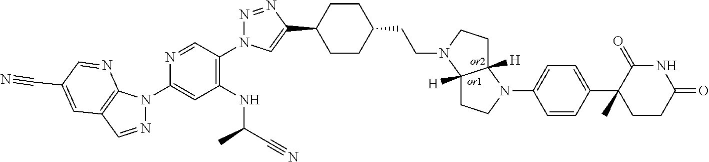 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc([C@@]4(C)CCC(=O)NC4=O)cc3)CC2)nn1] |
| P-414 | C-87 | 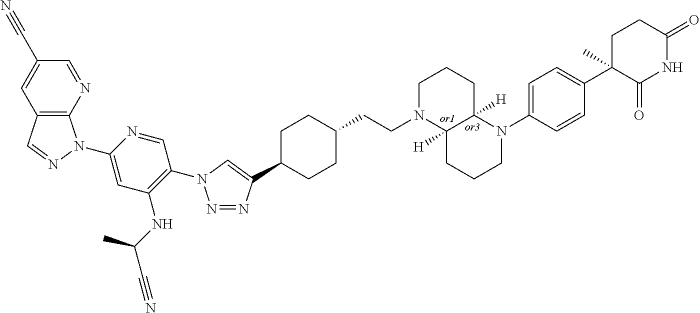 [SMILES: C[C@H](C#N)Nc1cc(-n2ncc3cc(C#N)cnc32)ncc1-n1cc([C@H]2CC[C@H](CCN3CCC[C@@H]4[C@H]3CCCN4c3ccc([C@@]4(C)CCC(=O)NC4=O)cc3)CC2)nn1] |
| P-487 | C-88b | 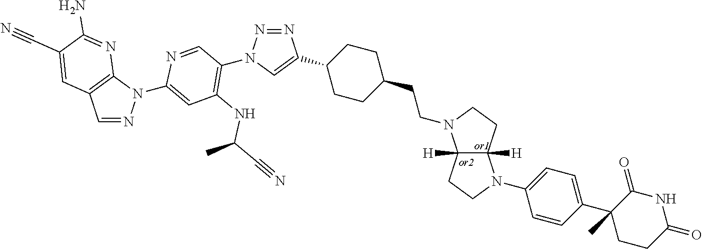 [SMILES: C[C@H](C#N)Nc1cc(-n2ncc3cc(C#N)c(N)nc32)ncc1-n1cc([C@H]2CC[C@H](CCN3CC[C@H]4[C@@H]3CCN4c3ccc([C@@]4(C)CCC(=O)NC4=O)cc3)CC2)nn1] |
| P-492 | C-88b | 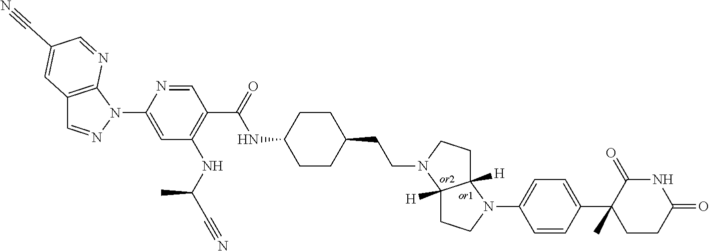 [SMILES: C[C@H](C#N)Nc1cc(-n2ncc3cc(C#N)cnc32)ncc1C(=O)N[C@H]1CC[C@H](CCN2CC[C@H]3[C@@H]2CCN3c2ccc([C@@]3(C)CCC(=O)NC3=O)cc2)CC1] |

| Compound | |
|---|---|
| No. | Characterization |
| P-246 | 15% yield as off white solid |
| | [M + H]⁺ = 779.8 |
| | 1.12-1.21 (m, 2H), 1.35-1.52 (m, 8H), 1.61- |
| | 1.68 (m, 2H), 1.73-1.77 (m, 2H), 1.86-1.89 |
| | (m, 2H), 2.01-2.17 (m, 4H), 2.40-2.45 (m, |
| | 1H), 2.71-2.78 (m, 1H), 2.94-3.01 (m, 2H), |
| | 3.09-3.17 (m, 2H), 3.22-3.24 (m, 2H), 3.60- |
| | 3.62 (m, 2H), 3.84-3.87 (m, 2H), 6.99-7.03 |
| | (m, 2H), 7.12-7.19 (m, 4H), 7.63 (s, 1H), 7.95 |
| | (s, 1H), 8.27 (d, J = 4.4 Hz, 2H), 8.31 (s, 1H), |
| | 8.60 (s, 1H), 10.88 (s, 1H) |
| | One proton is not apparent |
| P-301 | 16% yield as off white solid |
| | [M]+ = 778.4 |
| | ¹H NMR (400 MHz, DMSO-d6, 90° C.): δ ppm |
| | 1.18-1.28 (m, 5H), 1.42-1.46 (m, 6H), 1.53- |
| | 1.57 (m, 3H), 1.71-1.74 (m, 5H), 1.91-1.94 (m, |
| | 2H), 2.11-2.20 (m, 3H), 2.78-2.82 (m, 1H), |
| | 3.48-3.78 (m, 4H), 6.89-6.99 (m, 2H), 7.15- |
| | 7.21 (m, 2H), 7.58 (s, 1H), 8.05 (s, 1H), 8.24 (s, |
| | 1H), 8.43 (s 1H), 8.68 (s, 1H), 8.99 (d, J = 2.0 |
| | Hz, 1H), 9.05 (d, J = 2.0 Hz, 1H), 10.50 (s, 1H) |
| | Four protons are not apparent |
| P-298 | 12% yield as off white solid |
| | [M]+ = 727.4 |
| | ¹H NMR (400 MHz, DMSO-d6): δ ppm 1.11- |
| | 1.28 (m, 2H), 1.40 (s, 3H), 1.41-1.58 (m, 3H), |
| | 1.59-1.71 (m, 5H), 1.83-1.94 (m, 2H), 2.07- |
| | 2.12 (m, 5H), 2.72-2.78 (m, 1H), 2.92-3.06 (m, |
| | 2H), 3.15-3.38 (m, 4H), 3.59-3.63 (m, 2H), |
| | 3.84-3.87 (m, 2H), 4.69-4.76 (m, 1H), 6.76- |
| | 6.82 (m, 1H), 7.07-7.08 (m, 2H), 7.20 (d, J = 8.8 |
| | Hz, 2H), 7.45 (s, 1H), 7.87 (d, J = 10.2 Hz, 1H), |
| | 8.08-8.12 (m, 2H), 8.23 (s, 1H), 8.68 (d, J = 2.0 |
| | Hz, 1H), 10.52 (s, 1H), 10.88 (s, 1H) |
| | One proton is not apparent |
| P-251 | 35% yield as white solid |
| | [M + H]⁺ = 795.4 |
| | ¹H NMR (400 MHz, DMSO-d6): δ ppm 1.12- |
| | 1.22 (m, 3H), 1.36-1.40 (m, 9H), 1.45-1.55 |
| | (m, 4H), 1.63-1.68 (m, 5H), 1.91-1.94 (m, |
| | 2H), 2.02-2.08 (m, 2H), 2.13-2.17 (m, 2H), |
| | 2.71-2.82 (m, 2H), 3.01-3.09 (m, 1H), 3.13- |
| | 3.18 (m, 2H), 3.61-3.63 (m, 2H), 3.82-3.88 |
| | (m, 1H), 4.98-5.03 (m, 1H), 7.01 (d, J = 9.2 Hz, |
| | 2H), 7.09 (d, J = 8.4 Hz, 1H), 7.17 (d, J = 9.2 Hz, |
| | 2H), 7.36 (s, 2H), 8.00 (s, 1H), 8.25 (s, 1H), 8.33 |
| | (s, 1H), 8.35 (s, 1H), 8.59 (m, 1H), 10.88 (s, 1H). |
| | One proton is not apparent |
| P-269 | 14% yield as off white solid |
| | [M + H]+ = 849.2 |
| | ¹H NMR (400 MHz, DMSO-d6): δ ppm 1.12- |
| | 1.28 (m, 2H), 1.38-1.42 (m, 4H), 1.45-1.60 (m, |
| | 2H), 1.63 (d, J = 6.8 Hz, 3H), 1.63-1.64 (m, |
| | 2H), 1.87-1.91 (m, 3H), 1.97-2.08 (m, 2H), |
| | 2.14-2.22 (m, 2H), 2.33-2.47 (m, 1H), 2.72- |
| | 2.78 (m, 1H), 2.95-3.01 (m, 2H), 3.12-3.28 (m, |
| | 4H), 3.84-3.88 (m, 3H), 3.96-4.00 (m, 2H), |
| | 4.74 (s, 1H), 4.96-5.02 (m, 1H), 5.31 (s, 1H), |
| | 6.97-7.22 (m, 5H), 7.78 (s, 1H), 8.29 (s, 1H), |
| | 8.31-8.36 (m, 2H), 8.69 (s, 1H), 10.87 (s, 1H). |
| | Five protons are not apparent |
| P-247 | 1.4% yield |
| | [M + H]⁺ = 824.4 |
| | ¹H NMR (400 MHz, DMSO-d6): δ ppm 1.16- |
| | 1.24 (m, 3H), 1.38-1.41 (m, 1H), 1.40 (s, 3H), |
| | 1.49-1.60 (m, 2H), 1.62-1.67 (m, 5H), 1.87- |
| | 1.95 (m, 4H), 2.05-2.09 (m, 2H), 2.15-2.18 (m, |
| | 2H), 2.71-2.82 (m, 1H), 2.98-3.01 (m, 2H), |
| | 3.12-3.37 (m, 4H), 3.57-3.67 (m, 2H), 3.80- |
| | 3.86 (m, 4H), 4.72 (s, 1H), 4.91-4.98 (m, 1H), |
| | 5.12 (brs, 1H), 6.67 (brs, 1H), 7.00-7.05 (m, |
| | 3H), 7.18 (d, J = 8.8 2H), 7.99 (d, J = 8.8 Hz, |
| | 1H), 8.12-8.14 (m, 2H), 8.31 (s, 1H), 8.34 (s, |
| | 1H), 10.87 (s, 1H) |
| | Three protons are not apparent |
| P-297 | 40% yield pale white solid |
| | [M + H]+ = 761.3 |
| | ¹H NMR (400 MHz, DMSO-d6): δ ppm 1.11- |
| | 1.35 (m, 2H), 1.39-1.54 (m, 6H), 1.58-1.68 |
| | (m, 5H), 1.88 (d, J = 10.8, 2H), 2.00-2.09 (m, |
| | 2H), 2.12-2.16 (m, 2H), 2.41-2.45 (m, 1H), |
| | 2.71-2.79 (m, 1H), 2.92-3.01 (m, 2H), 3.09- |
| | 3.19 (m, 2H), 3.21-3.29 (m, 2H), 3.86 (d, J = |
| | 12.8 Hz, 2H), 4.68-4.77 (m, 1H), 6.93 (d, J = |
| | 8.0 Hz, 1H), 7.02 (d, J = 8.8 Hz, 2H), 7.18 (d, J = |
| | 8.8 Hz, 2H), 7.74 (s, 1H), 8.15 (s, 1H), 8.27 (s, |
| | 1H), 8.48 (d, J = 1.6 Hz, 1H), 8.64 (d, J = 2.0 Hz, |
| | 1H), 9.24 (s, 1H), 10.87 (s, 1H) |
| | 3 protons are not apparent |
| P-300 | 15% yield as pale yellow solid |
| | [M + H]⁺ = 728.4 |
| | ¹H NMR (400 MHz, DMSO-d6): δ ppm 1.11- |
| | 1.23 (m, 2H), 1.40-1.52 (m, 6H), 1.60-1.68 |
| | (m, 5H), 1.89-1.86 (m, 2H), 2.04-2.17 (m, |
| | 4H), 2.41-2.43 (m, 1H), 2.71-2.77 (m, 1H), |
| | 2.93-3.01 (m, 2H), 3.10-3.18 (m, 2H), 3.21- |
| | 3.27 (m, 2H), 3.58-3.61 (m, 2H), 3.82-3.87 |
| | (m, 2H), 4.76-4.82 (m, 1H), 6.93 (d, J = 7.6 Hz, |
| | 1H), 7.01 (d, J = 8.8 Hz, 2H), 7.18 (d, J = 8.8 Hz, |
| | 2H), 7.86 (s, 1H), 8.15 (s, 1H), 8.26 (s, 1H), 9.00 |
| | (s, 2H), 10.87 (s, 1H), 10.98 (s, 1H). |
| | One proton is not apparent |
| P-306 | 15% yield as off white solid |
| | [M + H]+ = 779.8 |
| | ¹H NMR (400 MHz, DMSO-d6): δ ppm 1.10- |
| | 1.25 (m, 2H), 1.35-1.60 (m, 6H), 1.65 (d, J = 7.2 |
| | Hz, 3H), 1.66-1.68 (m, 2H), 1.82-1.92 (m, 2H), |
| | 2.00-2.20 (m, 3H), 2.32-2.45 (m, 1H), 2.71- |
| | 2.82 (m, 1H), 3.02-3.30 (m, 6H), 3.58-3.70 (m, |
| | 2H), 3.92-3.95 (m, 2H), 4.98-5.03 (m, 1H), |
| | 7.08-7.12 (m, 1H), 7.23-7.34 (m, 2H), 7.38 |
| | (brs, 2H), 7.46-7.49 (m, 1H), 8.00 (s, 1H), 8.25 |
| | (s, 1H), 8.26-8.35 (m, 3H), 8.59 (s, 1H), 10.81 |
| | (s, 1H) |
| | One proton is not apparent |
| P-265 | 16% yield as |
| | ¹H NMR: (400 MHz, DMSO-d6, 100° C.): δ ppm |
| | 1.12-1.22 (m, 2H), 1.41-1.54 (m, 6H), 1.64- |
| | 1.71 (m, 5H), 1.87-1.89 (m, 2H), 2.04-2.22 (m, |
| | 4H), 2.34-2.41 (m, 1H), 2.74-2.78 (m, 1H), |
| | 3.19-3.23 (m, 2H), 3.78-3.82 (m, 2H), 4.92- |
| | 4.98 (m, 1H), 6.94 (d, J = 8.8 Hz, 2H), 7.57- |
| | 7.60 (m, 1H), 7.95 (brs, 1H), 8.06 (d, J = 2.4 Hz, |
| | 1H), 8.23 (brs, 2H), 8.40 (brs, 1H), 8.50 (s, 1H), |
| | 10.50 (s, 1H). |
| | Nine protons are not apparent |
| P-302 | 20% yield as off white solid |
| | [M + H]⁺ = 811.4 |
| | ¹H NMR (400 MHz, DMSO-d6): δ ppm 0.84 & |
| | 1.00 (d, J = 4.8 Hz, 3H), 1.38-1.55 (m, 6H), |
| | 1.63 (d, J = 6.8 Hz, 3H), 1.62-1.67 (m, 2H), |
| | 1.86-1.89 (m, 2H), 2.01-2.17 (m, 5H), 2.41- |
| | 2.44 (m, 2H), 2.72-2.78 (m, 2H), 3.12-3.21 (m, |
| | 5H), 3.41-3.62 (m, 4H), 3.81 (s, 3H), 4.99-5.03 |
| | (m, 1H), 6.76-6.78 (m, 1H), 6.89-6.98 (m, |
| | 1H), 7.07-7.11 (m, 2H), 7.38 (brs, 2H), 8.01 (s, |
| | 1H), 8.26 (s, 1H), 8.32 (s, 1H), 8.35 (s, 1H), 8.59 |
| | (s, 1H), 10.89 & 10.92 (s, 1H). |
| P-307 | 18% yield as yellow solid |
| | [M + H]+ = 768.3 |
| | ¹H NMR: (400 MHz, DMSO-d6) δ ppm: 1.12- |
| | 1.27 (m, 2H), 1.35-1.58 (m, 7H), 1.63-1.70 (m, |
| | 5H), 1.87-1.92 (m, 2H), 2.01-2.10 (m, 1H), |
| | 2.12-2.21 (m, 2H), 2.71-2.78 (m, 1H), 3.01- |
| | 3.10 (m, 2H), 3.12-3.29 (m, 4H), 3.88-3.99 (m, |
| | 2H), 4.97-5.08 (m, 1H), 7.09 (d, J = 9.4 Hz, |
| | 1H), 7.32-7.49 (m, 4H), 8.00 (s, 1H), 8.25 (s, |
| | 1H), 8.30-8.35 (m, 3H), 8.59 (s, 1H), 10.81 (s, |
| | 1H) |
| | Four protons are not apparent |
| P-266 | 25% yield as off white solid |
| | [M + H]⁺ = 768.4 |
| | ¹H NMR (400 MHz, DMSO, d6): δ ppm 1.11- |
| | 1.22 (m, 2H), 1.34-1.53 (m, 6H), 1.63-1.64 |
| | (m, 5H), 1.87-1.90 (m, 2H), 2.06-2.17 (m, |
| | 4H), 2.46-2.51 (m, 1H), 2.71-2.79 (m, 1H), |
| | 3.00-3.16 (m, 4H), 3.20-3.22 (m, 2H), 3.58- |
| | 3.62 (m, 2H), 4.38-4.41 (m, 2H), 4.99-5.03 |
| | (m, 1H), 7.00 (d, J = 8.8 Hz, 1H), 7.08-7.09 |
| | 1H), 7.37 (s, 2H), 7.58-7.61 (m, 1H), 8.00 (s, |
| | 1H), 8.06 (d, J = 2.4 Hz, 1H), 8.25 (s, 1H), 8.32 |
| | (s, 1H), 8.34 (s, 1H), 8.59 (s, 1H), 10.93 (s, 1H). |
| | One proton are not apparent |
| P-303 | 19% yield as off white solid |
| | [M]+ = 811.2 |
| | ¹H NMR (400 MHz, DMSO-d6): δ ppm 0.83 & |
| | 0.99 (d, J = 6.0 Hz, 3H), 1.16-1.19 (m, 2H), |
| | 1.38-1.52 (m, 6H), 1.63-1.64 (m, 5H), 1.87- |
| | 1.89 (m, 2H), 2.07-2.17 (m, 4H), 2.41-2.45 (m, |
| | 2H), 2.76-2.78 (m, 1H), 3.09-3.22 (m, 5H), |
| | 3.52-3.60 (m, 4H), 3.80 & 3.81 (s, 3H), 4.99- |
| | 5.03 (m, 1H), 6.78-6.79 (m, 1H), 6.89-6.97 (m, |
| | 1H), 7.06-7.1 (m, 2H), 7.37 (brs, 2H), 8.0 (s, |
| | 1H), 8.25 (s, 1H), 8.32 (m, 1H), 8.35 (s, 1H), 8.59 |
| | (s, 1H), 10.92 (s, 1H) |
| P-260 | 8.3% yield as white solid |
| | (M + H)+ = 781.3 |
| | ¹H NMR (400 MHz, DMSO-d6, 90° C.): δ ppm |
| | 0.91-1.18 (m, 3H), 1.21-1.28 (m, 2H), 1.46 (s, |
| | 3H), 1.51-1.58 (m, 2H), 1.67 (d, J = 6.8 Hz, |
| | 3H), 1.67-1.71 (m, 1H), 1.91-1.94 (m, 2H), |
| | 2.09-2.21 (m, 4H), 2.39-2.43 (m, 1H), 2.78- |
| | 2.82 (m, 1H), 3.55-3.67 (m, 2H), 4.36 (brs, 1H), |
| | 4.99-5.03 (m, 1H), 6.95-7.15 (m, 5H), 7.21 |
| | (brs, 2H), 7.95 (s, 1H), 8.22 (s, 1H), 8.28 (s, 1H), |
| | 8.37 (s, 1H), 8.53 (s, 1H), 10.50 (s, 1H |
| | Nine protons are not apparent |
| P-258 | 23% yield as off white solid |
| | [M + H]⁺ = 782.4 |
| | ¹H NMR (400 MHz, DMSO-d6): δ ppm 1.67- |
| | 1.24 (m, 6H), 1.38-1.52 (m, 6H), 1.63-1.69 (m, |
| | 5H), 1.87-1.89 (m, 2H), 2.06-2.17 (m, 5H), |
| | 2.72-2.78 (m, 1H), 2.94-3.08 (m, 1H), 3.15- |
| | 3.22 (m, 4H), 3.53-3.59 (m, 2H), 4.34-4.38 (m, |
| | 1H), 4.79-4.82 (m, 1H), 4.99-5.03 (m, 1H), |
| | 6.97-7.0 (m, 1H), 7.09-7.11 (m, 1H), 7.38 |
| | (brs, 2H), 7.57-7.59 (m, 1H), 8.01 (s, 1H), 8.05 |
| | (d, J = 2.0 Hz, 1H), 8.26 (s, 1H), 8.32 (s, 1H), |
| | 8.35 (s, 1H), 8.59 (s, 1H), 10.92 (s, 1H) |
| P-304 | 26% yield as off white solid |
| | [M]+ = 811.2 |
| | ¹H NMR (400 MHz, DMSO-d6): δ ppm 0.83 & |
| | 0.98 (d, J = 6.0 Hz, 3H), 1.18-1.23 (m, 2H), |
| | 1.31-1.43 (m, 1H), 1.44 (s, 3H), 1.46-1.52 (m, |
| | 2H), 1.62-1.64 (m, 5H), 1.86-1.89 (m, 2H), |
| | 2.07-2.17 (m, 4H), 2.42-2.46 (m, 1H), 2.71- |
| | 2.84 (m, 1H), 3.15-3.22 (m, 5H), 3.80 (s, 3H), |
| | 4.98-5.02 (m, 1H), 6.78-6.85 (m, 1H), 6.94- |
| | 6.96 (m, 1H), 7.06-7.09 (m, 2H), 7.36 (brs, 2H), |
| | 7.99 (s, 1H), 8.25 (s, 1H), 8.32 (m, 1H), 8.39 (s, |
| | 1H), 8.58 (s, 1H), 10.91 (s, 1H) |
| | Five protons are not apparent |
| P-259 | 13% yield as off white solid |
| | [M]+ = 782.4 |
| | ¹H NMR (400 MHz, DMSO-d6): δ ppm 1.18- |
| | 1.23 (m, 5H), 1.52 (s, 3H), 1.53-1.58 (m, 3H), |
| | 1.61-1.74 (m, 5H), 1.87-1.89 (m, 2H), 2.09- |
| | 2.13 (m, 1H), 2.14-2.18 (m, 2H), 2.71-2.84 |
| | (m, 1H), 2.95-3.08 (m, 1H), 3.15-3.24 (m, 4H), |
| | 4.31-3.41 (m, 1H), 4.71-4.82 (m, 1H), 4.99- |
| | 4.53 (m, 1H), 6.91-6.96 (m, 1H), 7.09 (d, J = |
| | 7.8, 1H), 7.37 (brs, 2H), 7.56-7.59 (m, 1H), 8.0 |
| | (s, 1H), 8.04 (d, J = 2.8, 1H), 8.25 (s, 1H), 8.32 |
| | (s, 1H), 8.35 (s, 1H), 8.56 (s, 1H), 10.93 (s, 1H) |
| | Five protons are not apparent |
| P-305 | 26% yield as off white solid |
| | [M + H]+ = 811.4 |
| | ¹H NMR (400 MHz, DMSO-d6): δ ppm 0.84 (d, |
| | J = 4.8 Hz, 3H), 1.11-1.27 (m, 2H), 1.38-1.53 |
| | (m, 6H), 1.63-1.66 (m, 5H), 1.86-1.90 (m, 2H), |
| | 2.08-2.17 (m, 4H), 2.41-2.45 (m, 1H), 2.72- |
| | 2.78 (m, 1H), 3.14-3.22 (m, 5H), 3.41-3.62 (m, |
| | 3H), 3.81 (s, 3H), 4.99-5.03 (m, 1H), 6.76- |
| | 6.78 (m, 1H), 6.89-6.97 (m, 1H), 7.06-7.11 |
| | (m, 2H), 7.37 (brs, 2H), 8.01 (s, 1H), 8.26 (s, |
| | 1H), 8.33 (s, 1H), 8.35 (s, 1H), 8.59 (s, 1H), |
| | 10.89 & 10.92 (s, 1H) |
| | Two protons are not apparent |
| P-261 | 36% yield as pale yellow solid |
| | [M + H]⁺ = 782.4 |
| | ¹H NMR (400 MHz, DMSO-d6): δ ppm 1.14- |
| | 1.20 (m, 2H), 1.23 (d, J = 6.8 Hz, 3H), 1.38- |
| | 1.58 (m, 6H), 1.64 (d, J = 6.8 Hz, 3H), 1.67-1.72 |
| | (m, 2H), 1.87-1.90 (m, 2H), 2.04-2.18 (m, 4H), |
| | 2.35-2.40 (m, 1H), 2.74-2.80 (m, 1H), 2.95- |
| | 3.05 (m, 1H), 3.16-3.23 (m, 4H), 3.53-3.59 (m, |
| | 2H), 4.35-4.40 (m, 1H), 4.78-4.81 (m, 1H), |
| | 4.99-5.04 (m, 1H), 6.93 (d, J = 9.2 Hz, 1H), |
| | 7.11 (d, J = 2.8 Hz, 1H), 7.38 (brs, 2H), 7.57- |
| | 7.60 (m, 1H), 8.01-8.05 (m, 2H), 8.26 (s, 1H), |
| | 8.33-8.35 (m, 2H), 8.59 (s, 1H), 10.93 (s, 1H), |
| | One proton is not apparent |
| P-262 | 45% yield as yellow solid |
| | (M + H)⁺ = 782.2 |
| | ¹H NMR (400 MHz, DMSO-d6): δ ppm 1.12- |
| | 1.22 (m, 5H), 1.38-1.55 (m, 6H), 1.63-1.67 |
| | (m, 5H), 1.85-1.89 (m, 2H), 2.02-2.17 (m, |
| | 4H), 2.32-2.38 (m, 1H), 2.72-2.78 (m, 1H), |
| | 2.96-3.02 (m, 1H), 3.15-3.23 (m, 4H), 4.32- |
| | 4.46 (m, 1H), 4.77-4.79 (m, 1H), 4.98-5.01 (m, |
| | 1H), 6.92 (d, J = 8.0 Hz, 1H), 7.12 (d, J = 8.0 Hz, |
| | 1H), 7.37 (brs, 2H), 7.57-7.60 (m, 1H), 8.01- |
| | 8.04 (m, 2H), 8.26 (s, 1H), 8.33 (d, J = 5.6 Hz, |
| | 2H), 8.58 (s, 1H), 10.93 (s, 1H). |
| | Three protons are not apparent |
| P-252 | 25% yield as white solid |
| | (M + H)⁺ = 796.4 |
| | ¹H NMR (400 MHz, DMSO-d6): δ ppm 1.17- |
| | 1.27 (m, 7H), 1.37 (d, J = 6.4 Hz, 3H), 1.44 (s, |
| | 3H), 1.49-1.57 (m, 2H), 1.63 (d, J = 6.8 Hz, |
| | 3H), 1.91-1.92 (m, 2H), 2.10-2.03 (m, 1H), |
| | 2.12-2.18 (m, 2H), 2.36-2.40 (m, 1H), 2.58- |
| | 2.63 (m, 1H), 2.72-2.80 (m, 1H), 2.98-3.09 |
| | (m, 2H), 3.58-3.62 (m, 1H), 3.88-4.01 (m, 1H), |
| | 4.15-4.32 (m, 2H), 4.99-5.03 (m, 1H), 7.00 (d, |
| | J = 9.2 Hz, 1H), 7.09 (d, J = 7.2 Hz, 1H), 7.37 (s, |
| | 2H), 7.56-7.59 (m, 1H), 8.00-8.02 (m, 2H), |
| | 8.25 (s, 1H), 8.33 (s, 1H), 8.35 (s, 1H), 8.59 (s, |
| | 1H), 10.93 (s, 1H). |
| | Four protons are not apparent |
| P-254 | 33% yield as white solid |
| | (M + H)⁺ = 796.4 |
| | ¹H NMR (400 MHz, DMSO-d6): δ ppm 1.17- |
| | 1.27 (m, 6H), 1.37 (d, J = 6.4 Hz, 3H), 1.38- |
| | 1.59 (m, 7H), 1.64 (d, J = 7.2 Hz, 3H), 1.91- |
| | 1.94 (m, 2H), 2.05-2.19 (m, 4H), 2.38-2.42 (m, |
| | 1H), 2.72-2.80 (m, 1H), 3.01-3.09 (m, 2H), |
| | 3.58-3.62 (m, 2H), 3.88-3.91 (m, 1H), 4.18- |
| | 4.28 (m, 2H), 4.99-5.03 (m, 1H), 7.00 (d, J = |
| | 8.8 Hz, 1H), 7.06 (d, J = 7.2 Hz, 1H), 7.37 (s, |
| | 2H), 7.56-7.59 (m, 1H), 8.00-8.02 (m, 2H), |
| | 8.25 (s, 1H), 8.33 (s, 1H), 8.35 (s, 1H), 8.59 (s, |
| | 1H), 10.93 (s, 1H) |
| | Two protons are not apparent |
| P-253 | 11% yield as pale yellow solid |
| | [M + H]⁺ = 796.4 |
| | ¹H NMR (400 MHz, DMSO-d6): δ ppm 1.18- |
| | 1.28 (m, 5H), 1.37-1.61 (m, 8H), 1.63 (d, J = 6.8 |
| | Hz, 6H), 1.91-1.94 (m, 2H), 2.02-2.18 (m, 4H), |
| | 2.34-2.40 (m, 1H), 2.71-2.82 (m, 1H), 2.98- |
| | 3.10 (m, 2H), 3.32-3.39 (m, 2H), 3.85-3.91 (m, |
| | 1H), 4.18-4.31 (m, 2H), 4.98-5.03 (m, 1H), |
| | 6.99-7.09 (m, 2H), 7.36 (brs, 2H), 7.56-7.59 |
| | (m, 1H), 8.00-8.02 (m, 2H), 8.25 (s, 1H), 8.32- |
| | 8.33 (m, 2H), 8.58 (s, 1H), 10.92 (s, 1H) |
| | Two protons are not apparent |
| P-255 | 22% yield as off white solid |
| | [M + H]+ = 796.2 |
| | ¹H NMR (400 MHz, DMSO-d6): δ ppm 1.19- |
| | 1.27 (m, 5H), 1.37-1.62 (m, 8H), 1.64 (d, J = 6.8 |
| | Hz, 6H), 1.88-1.94 (m, 2H), 2.00-2.20 (m, 4H), |
| | 2.34-2.40 (m, 1H), 2.72-2.79 (m, 1H), 2.99- |
| | 3.12 (m, 2H), 3.32-3.39 (m, 2H), 3.58-3.64 (m, |
| | 1H), 3.89-3.92 (m, 1H), 4.16-4.28 (m, 2H), |
| | 4.99-5.03 (m, 1H), 6.99-7.02 (m, 1H), 7.09- |
| | 7.11 (m, 1H), 7.38 (brs, 2H), 7.56-7.80 (m, 1H), |
| | 8.00-8.02 (m, 2H), 8.26 (s, 1H), 8.33-8.35 (m, |
| | 2H), 8.59 (s, 1H), 10.93 (s, 1H) |
| | One proton is not apparent |
| P-263 | 28% yield as off white solid |
| | [M + H]⁺ = 769.4 |
| | ¹H NMR (400 MHz, DMSO-d6): δ ppm 1.11- |
| | 1.22 (m, 2H), 1.36-1.39 (m, 1H), 1.43-1.49 (m, |
| | 2H), 1.53 (s, 3H), 1.64 (d, J = 7.2 Hz, 3H), 1.66- |
| | 1.68 (m, 2H), 1.86-1.90 (m, 2H), 2.00-2.09 |
| | (m, 2H), 2.13-2.17 (m, 2H), 2.28-2.33 (m, |
| | 1H), 2.41-2.47 (m, 1H), 2.72-2.79 (m, 1H), |
| | 3.09-3.27 (m, 6H), 3.60-3.63 (m, 2H), 3.99- |
| | 4.02 (m, 2H), 4.97-5.02 (m, 1H), 7.09 (d, J = |
| | 8.0 Hz, 1H), 7.38 (s, 2H), 8.01 (s, 1H), 8.25 (s, |
| | 1H), 8.35-8.32 (m, 2H), 8.57-8.59 (m, 3H), |
| | 10.83 (s, 1H) |
| P-264 | 26% yield as off white solid |
| | (M + H)⁺ = 769.4 |
| | ¹H NMR (400 MHz, DMSO-d6): δ ppm 1.12- |
| | 1.22 (m, 2H), 1.36-1.48 (m, 3H), 1.53 (s, 3H), |
| | 1.63 (d, J = 6.8 Hz, 3H), 1.65-1.67 (m, 2H), |
| | 1.87-1.90 (m, 2H), 2.00-2.09 (m, 2H), 2.14- |
| | 2.17 (m, 2H), 2.29-2.34 (m, 1H), 2.41-2.47 (m, |
| | 1H), 2.71-2.79 (m, 1H), 3.12-3.17 (m, 4H), |
| | 3.22-3.26 (m, 2H), 3.61-3.64 (m, 2H), 3.98- |
| | 4.02 (m, 2H), 4.97-5.02 (m, 1H), 7.08-7.11 |
| | (m, 1H), 7.37 (brs, 2H), 8.00 (s, 1H), 8.25 (s, |
| | 1H), 8.32 (s, 1H), 8.34 (s, 1H), 8.57-8.59 (m, |
| | 3H), 10.83 (s, 1H) |
| P-308 | 48% yield as off white solid |
| | [M + H]⁺ = 769.4 |
| | ¹H NMR (400 MHz, DMSO-d6): δ ppm 1.12- |
| | 1.25 (m, 2H), 1.38-1.53 (m, 6H), 1.64 (d, J = 6.8 |
| | Hz, 3H), 1.64-1.69 (m, 2H), 1.87-1.90 (m, 2H), |
| | 2.11-2.18 (m, 4H), 2.33-2.38 (m, 1H), 2.72- |
| | 2.78 (m, 1H), 3.08-3.16 (m, 2H), 3.23-3.31 (m, |
| | 4H), 3.63-3.66 (m, 2H), 4.48-4.51 (m, 2H), |
| | 4.99-5.05 (m, 1H), 7.09 (d, J = 8.0 Hz, 1H), |
| | 7.37 (brs, 2H), 7.48 (d, J = 9.6 Hz, 1H), 7.66 (d, J = |
| | 9.6 Hz, 1H), 8.00 (s, 1H), 8.25 (s, 1H), 8.33 (s, |
| | 1H), 8.34 (s, 1H), 8.59 (s, 1H), 10.88 (s, 1H) |
| | One proton is not apparent |
| P-309 | 42% yield as off white solid |
| | [M + H]⁺ = 769.3 |
| | ¹H NMR (400 MHz, DMSO-d6): δ ppm 1.12- |
| | 1.23 (m 2H), 1.35-1.41 (m, 1H), 1.46-1.53 |
| | (m, 5H), 1.64 (d, J = 7.2 Hz, 3H), 1.65-1.69 (m, |
| | 2H), 1.87-1.90 (m, 2H), 2.09-2.15 (m, 4H), |
| | 2.34-2.38 (m, 1H), 2.72-2.78 (m, 1H), 3.07- |
| | 3.14 (m, 2H), 3.21-3.23 (m, 4H), 3.63-3.65 |
| | (m, 2H), 4.48-4.51 (m, 2H), 4.98-5.02 (m, |
| | 1H), 7.07-7.09 (m, 1H), 7.34 (s, 2H), 7.56 (d, J = |
| | 10.0 Hz, 1H), 7.66 (d, J = 9.6 Hz, 1H), 8.00 (s, |
| | 1H), 8.25 (s, 1H), 8.32 (s, 1H), 8.34 (s, 1H), 8.59 |
| | (s, 1H), 10.88 (s, 1H) |
| | One proton is not apparent |
| P-294 | 30% yield as gummy solid |
| | [M + H]⁺ = 769.2 |
| | ¹H NMR (400 MHz, DMSO-d6): δ ppm 1.47 (s, |
| | 3H), 1.52-1.55 (m, 2H), 1.62 (d, J = 6.8 Hz, |
| | 3H), 1.67-1.79 (m, 4H), 1.89-1.92 (m, 2H), |
| | 2.01-2.18 (m, 4H), 2.41-2.46 (m, 1H), 2.82- |
| | 2.88 (m, 1H), 2.99-3.06 (m, 2H), 3.12-3.32 |
| | (m, 4H), 3.63-3.67 (m, 2H), 3.90-3.93 (m, |
| | 2H), 4.99-5.06 (m, 1H), 7.22 (d, J = 3.2 Hz, |
| | 1H), 7.32 (d, J = 8.8 Hz, 1H), 7.45-7.48 (m, |
| | 1H), 7.69 (s, 1H), 8.29 (d, J = 3.2 Hz, 1H), 8.38- |
| | 8.43 (m, 2H), 8.73 (s, 1H), 9.06-9.08 (m, 2H), |
| | 10.79 (1H) |
| | Two protons are not apparent |
| P-296 | 33% yield as off white solid |
| | [M + H]⁺ = 769.2 |
| | ¹H NMR (400 MHz, DMSO-d6): δ ppm 1.44 (s, |
| | 3H), 1.56-1.62 (m, 5H), 1.68-1.78 (m, 4H), |
| | 1.88-1.92 (m, 2H), 2.06-2.13 (m, 4H), 2.38- |
| | 2.42 (m, 1H), 2.87-2.92 (m, 1H), 3.09-3.16 (m, |
| | 4H), 3.20-3.32 (m, 2H), 3.63-3.65 (m, 2H), |
| | 4.34-4.41 (m, 2H), 5.01-5.05 (m, 1H), 6.96- |
| | 6.99 (m, 1H), 7.19-7.22 (m, 1H), 7.57-7.59 (m, |
| | 1H), 7.69 (s, 1H), 8.05 (d, J = 2.8 Hz, 1H), 8.39- |
| | 8.42 (s, 2H), 8.73 (s, 1H), 9.06-9.08 (s, 2H), |
| | 10.91 (s, 1H) |
| | Two protons are not apparent |
| P-267 | 34% yield as off white solid |
| | [M + H]⁺ = 769.3 |
| | ¹H NMR (400 MHz, DMSO-d6): δ ppm 1.14- |
| | 1.24 (m, 2H), 1.35-1.55 (m, 6H), 1.64 (d, J = 6.8 |
| | Hz, 3H), 1.65-1.70 (m, 1H), 1.87-1.90 (m, 2H), |
| | 2.04-2.28 (m, 4H), 2.35-2.42 (m, 1H), 2.75- |
| | 2.82 (m, 1H), 3.08-3.15 (m, 2H), 3.21-3.28 (m, |
| | 4H), 4.43-4.46 (m, 2H), 4.99-5.05 (m, 1H), |
| | 7.09 (d, J = 8.0 Hz, 1H), 7.36 (brs, 2H), 8.00 (s, |
| | 1H), 8.22 (s, 1H), 8.25 (s, 1H), 8.32-8.39 (m, |
| | 3H), 8.59 (s, 1H), 10.87 (s, 1H) |
| | Four protons are not apparent |
| P-268 | 52% yield as off white solid |
| | [M + H]+ = 769.2 |
| | ¹H NMR (400 MHz, DMSO-d6): δ ppm 1.12- |
| | 1.27 (m, 2H), 1.35-1.54 (m, 5H), 1.63-1.68 (m, |
| | 5H), 1.87-1.89 (m, 2H), 2.05-2.10 (m, 1H), |
| | 2.14-2.24 (m, 3H), 2.33-2.39 (m, 1H), 2.74- |
| | 2.79 (m, 1H), 3.08-3.15 (m, 2H), 3.21-3.36 (m, |
| | 4H), 3.61-3.64 (m, 2H), 4.43-4.46 (m, 2H), |
| | 4.99-5.05 (m, 1H), 7.10 (d, J = 8.0 Hz, 1H), |
| | 7.36 (brs, 2H), 8.00 (s, 1H), 8.22 (s, 1H), 8.25 (s, |
| | 1H), 8.33-8.39 (m, 3H), 8.59 (s, 1H), 10.87 (s, |
| | 1H) |
| | Two protons missing |
| P-293 | 21% yield as yellow solid |
| | [M + H]⁺ = 769.4 |
| | ¹H NMR (400 MHz, DMSO-d6): δ ppm 1.44- |
| | 1.49 (m, 5H), 1.62 (d, J = 6.8 Hz, 3H), 1.73- |
| | 1.76 (m, 2H), 1.81-1.91 (m, 6H), 2.01-2.11 (m, |
| | 2H), 2.31-2.43 (m, 1H), 2.72-2.83 (m, 1H), |
| | 3.12-3.20 (m, 4H), 3.22-3.35 (m, 2H), 4.38- |
| | 4.42 (m, 2H), 5.01-5.04 (m, 1H), 7.01 (d, J = 9.2 |
| | Hz, 1H), 7.20 (d, J = 8.4 Hz, 1H), 7.59-7.62 (m, |
| | 1H), 7.68 (s, 1H), 8.05 (d, J = 2.4 Hz, 1H), 8.37 |
| | (s, 1H), 8.42 (s, 1H), 8.72 (s, 1H), 9.06-9.08 (m, |
| | 2H), 10.93 (s, 1H) |
| | Four protons are not apparent |
| P-280 | 19% yield as off white solid |
| | (M + H)⁺ = 769.4 |
| | ¹H NMR (400 MHz, DMSO-d6): δ ppm 1.47 (s, |
| | 3H), 1.51-1.55 (m, 2H), 1.62 (d, J = 6.8 Hz, |
| | 3H), 1.67-1.77 (m, 4H), 1.89-1.92 (m, 2H), |
| | 2.00-2.13 (m, 4H), 2.36-2.42 (m, 1H), 2.85- |
| | 2.90 (m, 1H), 3.00-3.06 (m, 2H), 3.12-3.21 |
| | (m, 2H), 3.25-3.29 (m, 2H), 3.64-3.67 (m, |
| | 2H), 3.88-3.92 (m, 2H), 4.99-5.05 (m, 1H), |
| | 7.20 (d, J = 8.4 Hz, 1H), 7.31 (d, J = 8.8 Hz, 1H), |
| | 7.45-7.48 (m, 1H), 7.69 (s, 1H), 8.29 (d, J = 2.8 |
| | Hz, 1H), 8.39-8.41 (m, 2H), 8.73 (s, 1H), 9.06- |
| | 9.08 (m, 2H), 10.79 (s, 1H) |
| | Two protons are not apparent |
| P-295 | 13% yield as off white solid |
| | [M + H]+ = 769.4 |
| | ¹H NMR (400 MHz, DMSO-d6): δ ppm 1.45- |
| | 1.52 (m, 5H), 1.6 (d, J = 8.0 Hz, 3H), 1.72-1.91 |
| | (m, 8H), 2.01-2.18 (m, 2H), 2.72-2.80 (m, |
| | 1H), 3.01-3.09 (m, 2H), 3.12-3.22 (m, 2H), |
| | 3.62-3.65 (m, 2H), 3.91-3.95 (m, 2H), 4.46 |
| | (brs, 1H), 5.00-5.04 (m, 1H), 7.19 (d, J = 9.6 |
| | Hz, 1H), 7.31 (d, J = 8.8 Hz, 1H), 7.45-7.48 (m, |
| | 1H), 7.61 (s, 1H), 8.29 (d, J = 2.8 Hz, 1H), 8.37 |
| | (s, 1H), 8.42 (s, 1H), 8.72 (s, 1H), 9.05-9.07 (m, |
| | 2H), 10.79 (s, 1H) |
| | Four protons are not apparent |
| P-256 | 46% yield as off white solid |
| | [M + H]⁺ = 782.4 |
| | ¹H NMR (400 MHz, DMSO-d6, 100° C.): δ ppm |
| | 1.19-1.22 (m, 4H), 1.48-1.56 (m, 6H), 1.63- |
| | 1.68 (m, 5H), 1.89-1.91 (m, 2H), 1.95-2.13 (m, |
| | 1H), 2.15-2.22 (m, 3H), 2.39-2.44 (m, 2H), |
| | 2.73-2.85 (m, 1H), 3.18-3.22 (m, 2H), 3.23- |
| | 3.38 (m, 2H), 4.91-5.03 (m, 1H), 6.89-6.91 |
| | (m, 2H), 7.29-7.41 (m, 2H), 7.92 (s, 1H), 8.19 |
| | (s, 1H), 8.24-8.26 (m, 2H), 8.35 (s, 1H), 8.49 (s, |
| | 1H), 10.34 (s, 1H) |
| | Seven protons are not apparent |
| P-312 | 7% yield as pale yellow solid |
| | [M + H]⁺ = 779.4 |
| | ¹H NMR (400 MHz, DMSO-d6): δ ppm 1.18- |
| | 1.24 (m, 2H), 1.44 (s, 3H), 1.45-1.53 (m, 3H), |
| | 1.61 (d, J = 7.8 Hz, 3H), 1.65-1.68 (m, 2H), |
| | 1.72-1.91 (m, 3H), 2.05-2.09 (m, 2H), 2.15- |
| | 2.18 (m, 3H), 2.58-2.65 (m, 1H), 2.76-2.82 (m, |
| | 1H), 3.24-3.29 (m, 2H), 3.38-3.43 (m, 1H), |
| | 3.58-3.64 (m, 2H), 4.25-4.27 (m, 1H), 4.68- |
| | 4.69 (m, 1H), 5.00-5.04 (m, 1H), 6.67 (d, J = 8.4 |
| | Hz, 1H), 7.20 (d, J = 4.8 Hz, 1H), 7.57 (d, J = |
| | 8.8 Hz, 1H), 7.68 (s, 1H), 7.97-8.0 (m, 1H), |
| | 8.37 (s, 1H), 8.41 (s, 1H), 8.72 (s, 1H), 9.06- |
| | 9.07 (m, 2H), 10.91 (s, 1H) |
| | Four protons missing |
| P-289 | 21% yield as off white solid |
| | [M + H]⁺ = 767.4 |
| | ¹H NMR (400 MHz, DMSO-d6, 100° C.): δ ppm |
| | 1.19-1.23 (m, 5H), 1.38-1.46 (m, 1H), 1.50 (s, |
| | 3H), 1.52-1.56 (m, 2H), 1.63-1.70 (m, 5H), |
| | 1.89-1.92 (m, 2H), 1.95-2.08 (m, 1H), 2.16- |
| | 2.22 (m, 3H), 2.39-2.46 (m, 2H), 2.73-2.85 (m, |
| | 1H), 3.19-3.23 (m, 2H), 3.25-3.38 (m, 2H), |
| | 4.90-4.94 (m, 1H), 7.03 (d, J = 0.8, 1H), 7.29- |
| | 7.41 (m, 2H), 7.71 (s, 1H), 8.25-8.28 (m, 2H), |
| | 8.43 (s, 1H), 8.63 (s, 1H), 8.94 (d, J = 2.0, 1H), |
| | 8.98 (d, J = 2.0, 1H), 10.34 (s, 1H) |
| | Five protons are not apparent |
| P-257 | 52% yield as off white solid |
| | [M]+ = 782.4 |
| | ¹H NMR (400 MHz, DMSO-d6, 100° C.): δ ppm |
| | 1.18-1.23 (m, 5H), 1.35-1.48 (m, 1H), 1.52 (s, |
| | 3H), 1.53-1.56 (m, 2H), 1.64-1.66 (m, 5H), |
| | 1.89-1.91 (m, 2H), 1.95-2.08 (m, 1H), 2.15- |
| | 2.26 (m, 3H), 2.41-2.45 (m, 2H), 2.75-2.84 |
| | (m, 1H), 3.15-3.38 (m, 4H), 4.91-4.98 (m, 1H), |
| | 6.89 (d, J = 7.6, 2H), 7.31 (brs, 1H), 7.40 (brs, |
| | 1H), 7.92 (s, 1H), 8.19 (s, 1H), 8.24-8.25 (m, |
| | 2H), 8.35 (s, 1H), 8.49 (s, 1H), 10.34 (s, 1H) |
| | Six protons are not apparent |
| P-290 | 22% yield as off white solid |
| | [M + H]⁺ = 767.2 |
| | ¹H NMR (400 MHz, DMSO-d6, 100° C.): δ ppm |
| | 1.12-1.25 (m, 5H), 1.43-1.51 (m, 1H), 1.52 (s, |
| | 3H), 1.52-1.58 (m, 2H), 1.66 (d, J = 7.2 Hz, |
| | 3H), 1.67-1.71 (m, 2H), 1.91-1.94 (m, 2H), |
| | 2.01-2.08 (m, 1H), 2.19-2.28 (m, 3H), 2.42- |
| | 2.46 (m, 2H), 2.78-2.85 (m, 1H), 3.21-3.25 (m, |
| | 2H), 3.31-3.36 (m, 2H), 4.90-4.98 (m, 1H), |
| | 7.05 (d, J = 8.0, 1H), 7.32 (d, J = 8.4 Hz, 1H), |
| | 7.43 (brs, 1H), 7.74 (s, 1H), 8.28 (brs, 1H), 8.30 |
| | (s, 1H), 8.45 (s, 1H), 8.65 (s, 1H), 8.96 (d, J = |
| | 2.0, 1H), 9.00 (d, J = 2.0, 1H), 10.36 (s, 1H) |
| | Five protons are not apparent |
| P-286 | 28% yield as off white solid |
| | [M]+ = 778.7 |
| | ¹H NMR (400 MHz, DMSO-d6): δ ppm 1.12- |
| | 1.28 (m, 2H), 1.46-1.52 (m, 6H), 1.60-1.66 (m, |
| | 5H), 1.88-1.91 (m, 2H), 2.03-2.18 (m, 3H), |
| | 2.72-2.82 (m, 1H), 3.21-3.39 (m, 4H), 4.15- |
| | 4.32 (m, 1H), 4.43-4.56 (m, 1H), 5.01-5.04 (m, |
| | 1H), 7.01-7.12 (m, 1H), 7.18-7.27 (m, 2H), |
| | 7.68 (s, 1H), 7.98 (d, J = 2.8 Hz, 1H), 8.37 (s, |
| | 1H), 8.41 (s, 1H), 8.72 (s, 1H), 9.05-9.07 (m, |
| | 2H), 10.76 (s, 1H) |
| | Nine protons are not apparent |
| P-287 | 26% yield as off white solid |
| | [M + H]⁺ = 779.4 |
| | ¹H NMR (400 MHz, DMSO-d6): δ ppm 1.18- |
| | 1.21 (m, 2H), 1.47-1.53 (m, 6H), 1.61-1.67 (m, |
| | 5H), 1.88-1.91 (m, 2H), 2.07-2.08 (m, 1H), |
| | 2.13-2.19 (m, 2H), 2.32-2.35 (m, 2H), 2.39- |
| | 2.46 (m, 1H), 2.67-2.68 (m, 1H), 2.78-2.82 (m, |
| | 1H), 3.24-3.29 (m, 2H), 3.36-3.49 (m, 2H), |
| | 3.51-3.61 (m, 1H), 3.62-3.79 (m, 2H), 4.15- |
| | 4.31 (m, 1H), 4.53-4.56 (m, 1H), 5.01-5.05 (m, |
| | 1H), 7.19-7.21 (m, 1H), 7.22-7.28 (m, 1H), |
| | 7.26-7.29 (m, 1H), 7.68 (s, 1H), 7.99 (d, J = |
| | 2.8 Hz, 1H), 8.38 (s, 1H), 8.42 (s, 1H), 8.73 (s, |
| | 1H), 9.06-9.07 (m, 2H), 10.78 (s, 1H) |
| | Two protons are not apparent |
| P-313 | 31% yield as off white solid |
| | [M + H]⁺ = 779.2 |
| | ¹H NMR (400 MHz, DMSO-d6): δ ppm 1.18- |
| | 1.23 (m, 2H), 1.43-1.52 (m, 6H), 1.60-1.67 (m, |
| | 5H), 1.78-1.84 (m, 1H), 1.88-1.91 (m, 2H), |
| | 2.05-2.18 (m, 4H), 2.74-2.79 (m, 1H), 3.24- |
| | 3.29 (m, 3H), 4.23-4.25 (m, 1H), 4.46-4.51 |
| | (m, 1H), 4.66-4.70 (m, 1H), 4.98-5.04 (m, |
| | 1H), 6.66 (d, J = 9.2 Hz, 1H), 7.18 (d, J = 8.4 Hz, |
| | 1H), 7.4 (d, J = 8.0 Hz, 1H), 7.67 (s, 1H), 7.98- |
| | 8.02 (m, 1H), 8.37 (s, 1H), 8.41 (s, 1H), 8.72 (s, |
| | 1H), 9.06-9.07 (m, 2H), 10.90 (s, 1H) |
| | Seven protons are not apparent |
| P-405 | 29% yield as pale yellow solid |
| | [M + H]⁺ = 755.4 |
| | ¹H NMR (400 MHz, DMSO-d6): δ ppm 1.10- |
| | 1.25 (m, 2H), 1.35-1.55 (m, 3H), 1.62-1.68 (m, |
| | 2H), 1.87-2.05 (m, 4H), 2.14-2.17 (m, 3H), |
| | 2.28-2.32 (m, 1H), 2.62-2.80 (m, 2H), 2.92- |
| | 3.01 (m, 2H), 3.12-3.28 (m, 4H), 3.81-3.95 (m, |
| | 5H), 4.22-4.28 (m, 1H), 6.97-6.99 (m, 3H), |
| | 7.12 (d, J = 8.8 Hz, 2H), 7.56 (s, 1H), 8.38 (s, |
| | 1H), 8.43 (s, 1H), 8.68 (s, 1H), 9.04 (d, J = 2.0 |
| | Hz, 1H), 9.08 (d, J = 2.0 Hz, 1H), 10.79 (s, 1H) |
| | Five protons not apparent |
| P-409 | 9% as off white solid |
| | 1H NMR (400 MHz, DMSO-d6): δ ppm 0.88- |
| | 0.95 (m, 4H), 1.11-1.23 (m, 2H), 1.37-1.52 |
| | (m, 3H), 1.62-1.68 (m, 2H), 1.87-1.89 (m, |
| | 2H), 1.98-2.13 (m, 1H), 2.09-2.21 (m, 3H), |
| | 2.61-2.68 (m, 1H), 2.73-2.80 (m, 1H), 2.94- |
| | 3.01 (m, 2H), 3.11-3.27 (m, 4H), 3.61-3.65 (m, |
| | 2H), 3.74-3.79 (m, 1H), 3.83-3.87 (m, 2H), |
| | 4.22-4.28 (m, 1H), 6.99 (d, J = 8.8 Hz, 2H), |
| | 7.13 (d, J = 8.8 Hz, 2H), 8.24 (s, 1H), 8.34 (d, J = |
| | 3.2 Hz, 1H), 8.77-8.78 (m, 2H), 9.09 (d, J = 2.0 |
| | Hz, 1H), 9.13 (d, J = 2.0 Hz, 1H), 10.80 (s, 1H). |
| | One proton not apparent |
| P-406 | 17% yield as off white solid |
| | [M + H]⁺ = 769.3 |
| | ¹H NMR: (400 MHz, DMSO-d6) δ ppm: 1.12- |
| | 1.28 (m, 2H), 1.36-1.70 (m, 8H), 1.85-2.05 (m, |
| | 5H), 2.11-2.20 (m, 3H), 2.62-2.82 (m, 2H), |
| | 2.91-3.02 (m, 2H), 3.12-3.30 (m, 4H), 3.60- |
| | 3.64 (m, 2H), 3.74-3.88 (m, 6H), 6.75 (d, J = |
| | 7.6 Hz, 1H), 6.98 (d, J = 8.8 Hz, 2H), 7.12 (d, J = |
| | 8.8 Hz, 2H), 7.59 (s, 1H), 8.36 (s, 1H), 8.43 (s, |
| | 1H), 8.67 (s, 1H), 9.04-9.07 (m, 2H), 10.79 (s, |
| | 1H) |
| | Two protons not apparent |
| P-408 | 4% yield as off white solid |
| | [M + H]⁺ = 755.4 |
| | ¹H NMR (400 MHz, DMSO-d6): δ ppm 1.19- |
| | 1.21 (m, 2H), 1.41-1.52 (m, 6H), 1.82-1.91 (m, |
| | 2H), 1.85-1.92 (m, 2H), 1.98-2.05 (m, 1H), |
| | 2.12-2.20 (m, 3H), 2.39-2.42 (m, 1H), 2.61- |
| | 2.68 (m, 1H), 2.72-2.78 (m, 1H), 3.11-3.14 (m, |
| | 4H), 3.56-3.58 (m, 2H), 3.70-3.75 (m, 1H), |
| | 4.32-4.35 (m, 2H), 4.64-4.68 (m, 2H), 6.89- |
| | 6.91 (m, 2H), 6.95-6.98 (m, 1H), 7.05-7.07 |
| | (m, 2H), 7.48 (s, 1H), 8.28 (s, 1H), 8.32 (s, 1H), |
| | 8.67 (m, 1H), 9.04-9.08 (m, 2H), 10.76 (s, 1H). |
| | Four protons are not apparent |
| P-395 | 22% yield as off white solid |
| | [M + H]+ = 713.3 |
| | ¹H NMR (400 MHz, DMSO-d6): δ ppm 1.18- |
| | 1.24 (m, 6H), 1.38-1.58 (m, 3H), 1.62-1.72 (m, |
| | 2H), 1.88-1.92 (m, 2H), 1.99-2.04 (m, 1H), |
| | 2.15-2.18 (m, 2H), 2.62-2.68 (m, 1H), 2.75- |
| | 2.83 (m, 1H), 2.95-2.99 (m, 2H), 3.11-3.43 |
| | (m, 6H), 3.61-3.64 (m, 2H), 3.76-3.80 (m, 1H), |
| | 3.83-3.87 (m, 2H), 6.82 (brs, 1H), 6.99 (d, J = |
| | 8.8 Hz, 2H), 7.13 (d, J = 8.8 Hz, 2H), 7.46 (s, |
| | 1H), 8.29 (s, 1H), 8.35 (s, 1H), 8.67 (s, 1H), 9.04- |
| | 9.07 (m, 2H), 10.79 (s, 1H) |
| | One proton not apparent |
| P-299 | 34% yield as pale yellow solid |
| | [M + H]⁺ = 745.2 |
| | ¹H NMR (400 MHz, DMSO-d6): δ ppm 1.12- |
| | 1.24 (m, 2H), 1.40-1.52 (m, 6H), 1.59-1.68 |
| | (m, 5H), 1.88 (d, J = 10.4 Hz, 2H), 2.04-2.16 |
| | (m, 4H), 2.71-2.78 (m, 1H), 2.94-3.01 (m, |
| | 2H), 3.11-3.19 (m, 2H), 3.21-3.28 (m, 2H), |
| | 3.58-3.62 (m, 2H), 3.72-3.78 (m, 2H), 4.76- |
| | 4.83 (m, 1H), 6.90 (d, J = 7.6 Hz, 1H), 7.02 (d, J = |
| | 9.2 Hz, 2H), 7.18 (d, J = 8.8, 2H), 7.71 (s, 1H), |
| | 8.14 (s, 1H), 8.23 (d, J = 2.0 Hz, 1H), 8.26 (d, J = |
| | 2.8 Hz, 1H), 8.53 (d, J = 2.0 Hz, 1H), 10.15 (s, |
| | 1H), 10.87 (s, 1H). |
| | One proton is not apparent |
| P-377 | 3% yield as off white solid |
| | [M + H]⁺ = 739.3 |
| | ¹H NMR (400 MHz, DMSO-d6): δ ppm 1.13- |
| | 1.24 (m, 3H), 1.37-1.41 (m, 1H), 1.47-1.55 (m, |
| | 2H), 1.67-1.68 (m, 5H), 1.89-1.91 (m, 2H), |
| | 1.98-2.03 (m, 1H), 2.18-2.16 (m, 3H), 2.61- |
| | 2.63 (m, 1H), 2.73-2.80 (m, 1H), 2.92-3.00 |
| | (m, 2H), 3.11-3.19 (m, 2H), 3.60-3.63 (m, |
| | 2H), 3.72-3.78 (m, 1H), 3.81-3.87 (m, 2H), |
| | 5.38-5.42 (m, 1H), 6.98-7.00 (m, 2H), 7.11- |
| | 7.13 (m, 2H), 8.42-8.39 (m, 2H), 8.68 (s, 1H), |
| | 8.73 (s, 1H), 9.05 (d, J = 2.0 Hz, 1H), 9.12 (d, J = |
| | 2.0 Hz, 1H), 10.79 (s, 1H) |
| | Two protons are not apparent |
| P-392 | 23% yield as off white solid |
| | [M + H]⁺ = 752.3 |
| | ¹H NMR (400 MHz, DMSO-d6): δ ppm 1.03 (t, |
| | J = 7.2 Hz, 3H), 1.17-1.22 (m, 2H), 1.36-1.52 (m, |
| | 3H), 1.66-1.68 (m, 2H), 1.87-1.90 (m, 2H), |
| | 1.96-2.02 (m, 3H), 2.14-2.17 (m, 3H), 2.44- |
| | 2.48 (m, 1H), 2.65-2.71 (m, 1H), 2.73-2.84 |
| | (m, 1H), 2.95-3.06 (m, 2H), 3.14-3.24 (m, 4H), |
| | 3.60-3.63 (m, 2H), 3.75-3.85 (m, 3H), 4.87- |
| | 4.98 (m, 1H), 6.97-7.02 (m, 2H), 7.11-7.15 (m, |
| | 2H), 7.20 (d, J = 8.4 Hz, 1H), 7.69 (s, 1H), 8.38 |
| | (s, 1H), 8.42 (s, 1H), 8.71 (s, 1H), 9.04-9.07 (m, |
| | 2H), 10.78 (s, 1H) |
| P-402 | 4% yield as off white solid |
| | [M + H]⁺ = 769.3 |
| | ¹H NMR (400 MHz, DMSO-d6): δ ppm 1.12- |
| | 1.22 (m, 2H), 1.39-1.70 (m, 7H), 1.75-1.90 |
| | (m, 4H), 1.99-2.02 (1H), 2.12-2.18 (m, 4H), |
| | 2.61-2.65 (m, 1H), 2.73-2.80 (m, 1H), 2.97- |
| | 3.02 (m, 2H), 3.12-3.24 (m, 4H), 3.73-3.83 |
| | (m, 4H), 4.13 (brs, 1H), 5.17 (brs, 1H), 6.97- |
| | 7.00 (m, 2H), 7.10-7.14 (m, 3H), 7.52 (s, 1H), |
| | 8.45 (s, 1H), 8.50 (s, 1H), 8.68 (s, 1H), 9.04 (d, J = |
| | 2.0 Hz, 1H), 9.07 (d, J = 2.0 Hz, 1H), 10.79 (s, |
| | 1H) |
| | Four protons are not apparent |
| P-397 | 4% yield as off white solid |
| | [M + H]⁺ = 743.4 |
| | ¹H NMR (400 MHz, DMSO-d6): δ ppm 1.18- |
| | 1.25 (m, 3H), 1.49-1.52 (m, 3H), 1.62-1.68 (m, |
| | 5H), 1.88-1.91 (m, 2H), 1.98-2.12 (m, 1H), |
| | 2.15-2.18 (m, 3H), 2.62-2.78 (m, 2H), 2.95- |
| | 3.05 (m, 2H), 3.14-3.34 (m, 4H), 3.60-3.63 (m, |
| | 2H), 3.75-3.79 (m, 1H), 3.82-3.87 (m, 2H), |
| | 3.92 (s, 3H), 4.98-5.02 (m, 1H), 6.98-7.00 (m, |
| | 2H), 7.06-7.14 (m, 3H), 7.77 (s, 1H), 7.89 (d, J = |
| | 2.8 Hz, 1H), 8.34-8.36 (m, 2H), 8.42 (s, 1H), |
| | 8.46 (d, J = 3.2 Hz, 1H), 10.79 (s, 1H) |
| P-403 | 16% yield as off white solid |
| | [M + H]⁺ = 769.4 |
| | ¹H NMR (400 MHz, DMSO-d6): δ ppm 1.11- |
| | 1.21 (m, 2H), 1.38-1.53 (m, 5H), 1.61-1.74 |
| | (m, 4H), 1.80-1.91 (m, 3H), 1.98-2.03 (m, |
| | 1H), 2.13-2.23 (m, 4H), 2.60-2.69 (m, 1H), |
| | 2.71-2.79 (m, 1H), 2.92-3.00 (m, 2H), 3.11- |
| | 3.29 (m, 4H), 3.56-3.67 (m, 3H), 3.92-3.98 |
| | (m, 1H), 6.80 (d, J = 6.8 Hz, 1H), 6.96-7.00 (m, |
| | 2H), 7.09-7.13 (m, 2H), 7.59 (s, 1H), 8.38 (s, |
| | 1H), 8.45 (s, 1H), 8.67 (s, 1H), 9.03-9.07 (m, |
| | 2H), 10.78 (s, 1H). |
| | Five protons are not apparent |
| P-410 | 9% yield as off white solid |
| | [M + H]⁺ = 754.2 |
| | ¹H NMR (400 MHz, DMSO-d6): δ ppm 1.11- |
| | 1.27 (m, 2H), 1.46-1.53 (m, 3H), 1.61-1.72 (m, |
| | 6H), 1.86-1.90 (m, 4H), 1.98-2.03 (m, 3H), |
| | 2.09-2.13 (m, 3H), 2.62-2.64 (m, 1H), 2.72- |
| | 2.82 (m, 1H), 2.91-3.02 (m, 2H), 3.14-3.32 (m, |
| | 4H), 3.61-3.64 (m, 2H), 3.83-3.87 (m, 2H), |
| | 5.18-5.22 (m, 1H), 6.96-7.00 (m, 2H), 7.09- |
| | 7.14 (m, 2H), 8.03 (s, 1H), 8.23 (s, 1H), 8.75 (s, |
| | 1H), 8.78 (s, 1H), 9.08 (d, J = 2.0 Hz, 1H), 9.12 |
| | (d, J = 2.0 Hz, 1H), 10.80 (s, 1H) |
| | Two protons are not apparent |
| P-411 | 16% yield as off white solid |
| | [M + H]⁺ = 784.4 |
| | ¹H NMR (400 MHz, DMSO-d6): δ ppm 1.15- |
| | 1.25 (m, 2H), 1.27-1.53 (m, 5H), 1.55-1.69 (m, |
| | 4H), 1.86-1.89 (m, 2H), 1.99-2.20 (m, 5H), |
| | 2.62-2.66 (m, 1H), 2.76-2.82 (m, 1H), 2.95- |
| | 3.00 (m, 2H), 3.12-3.20 (m, 2H), 3.24-3.29 (m, |
| | 4H), 3.61-3.64 (m, 2H), 3.75-3.79 (m, 1H), |
| | 3.84-3.88 (m 4H), 4.20 (d, J = 6.0 Hz, 1H), |
| | 6.96-7.00 (m, 2H), 7.09-7.14 (m, 2H), 8.00 (s, |
| | 1H), 8.27 (s, 1H), 8.74-8.78 (m, 2H), 9.07-9.12 |
| | (m, 2H), 10.79 (s, 1H) |
| | Two protons are not apparent |
| P-375 | 6% yield as off white solid |
| | [M + H]⁺ = 747.3 |
| | ¹H NMR (400 MHz, DMSO-d6): δ ppm 1.12- |
| | 1.25 (m, 2H), 1.35-1.60 (m, 3H), 1.61-1.67 (m, |
| | 4H), 1.86-1.90 (m, 1H), 1.98-2.01 (m, 1H), |
| | 2.15-2.18 (m, 3H), 2.62-2.82 (m, 2H), 2.98- |
| | 3.04 (m, 2H), 3.15-3.18 (m, 2H), 3.26-3.28 |
| | (m, 2H), 3.60-3.63 (m, 2H), 3.75-3.79 (m, 1H), |
| | 3.83-3.86 (m, 2H), 4.98-5.06 (m, 1H), 6.99 (d, |
| | J = 8.8 Hz, 2H), 7.12-7.15 (m, 3H), 7.70 (s, 1H), |
| | 8.36 (s, 1H), 8.39 (s, 1H), 8.53 (s, 1H), 8.58 (s, |
| | 1H), 8.72 (s, 1H), 10.79 (s, 1H) |
| | Three protons are not apparent |
| P-391 | 6% as brown solid |
| | [M + H]⁺ = 757.2 |
| | ¹H NMR (400 MHz, DMSO-d6): δ ppm 1.12- |
| | 1.23 (m, 8H), 1.37-1.54 (m, 3H), 1.61-1.68 (m, |
| | 2H), 1.88 (d, J = 10.8 Hz, 2H), 1.97-2.03 (m, |
| | 1H), 2.12-2.18 (m, 3H), 2.72-2.81 (m, 1H), |
| | 2.91-2.99 (m, 2H), 3.11-3.29 (m, 6H), 3.61- |
| | 3.64 (m, 4H), 3.66-3.88 (m, 3H), 4.85-4.95 |
| | (brs, 1H), 6.90-6.92 (s, 1H), 6.92-7.03 (m, |
| | 2H), 7.05-7.17 (m, 2H), 7.51 (s, 1H), 8.43 (s, |
| | 1H), 8.51 (s, 1H), 8.67 (s, 1H), 9.04-9.07 (m, |
| | 2H), 10.81 (s, 1H). |
| P-380 | 12% yield as white solid |
| | (M + H)⁺ = 756.3 |
| | ¹H NMR (400 MHz, DMSO-d6): δ ppm 1.12- |
| | 1.22 (m, 2H), 1.36-1.40 (m, 1H), 1.42-1.53 (m, |
| | 2H), 1.60 (d, J = 6.8 Hz, 3H), 1.61-1.69 (m, |
| | 2H), 1.85-1.91 (m, 2H), 1.98-2.08 (m, 1H), |
| | 2.12-2.21 (m, 3H), 2.60-2.63 (m, 1H), 2.69- |
| | 2.78 (m, 1H), 2.92-3.00 (m, 2H), 3.11-3.18 |
| | (m, 2H), 3.21-3.28 (m, 2H), 3.59-3.63 (m, |
| | 2H), 3.76-3.80 (m, 1H), 3.81-3.88 (m, 2H), |
| | 4.78-4.82 (m, 1H), 6.83 (d, J = 7.6 Hz, 1H), |
| | 6.99 (d, J = 10.8 Hz, 2H), 7.13 (d, J = 8.8 Hz, |
| | 2H), 7.62 (s, 1H), 8.07 (s, 1H), 8.23 (d, J = 2.4 |
| | Hz, 2H), 8.31 (d, J = 2.0 Hz, 1H), 8.85 (s, 1H), |
| | 10.79 (s, 1H). |
| | One proton is not apparent |
| P-388 | 4% yield as a off white solid |
| | (M + H)⁺ = 714.4 |
| | ¹H NMR (400 MHz, DMSO-d6): δ ppm 1.11- |
| | 1.22 (m, 2H), 1.35-1.52 (m, 3H), 1.61 (d, J = |
| | 6.8 Hz, 3H), 1.62-1.67 (m, 2H), 1.86-1.89 (m, |
| | 2H), 1.97-2.03 (m, 1H), 2.12-2.17 (m, 3H), |
| | 2.60-2.76 (m, 2H), 2.93-3.00 (m, 2H), 3.07- |
| | 3.17 (m, 2H), 3.23-3.26 (m, 2H), 3.60-3.63 |
| | (m, 2H), 3.75-3.79 (m, 1H), 3.82-3.86 (m, |
| | 2H), 4.76-4.83 (m, 1H), 6.93 (d, J = 8.0 Hz, |
| | 1H), 6.98-7.00 (m, 2H), 7.12-7.14 (d, J = 8.8 |
| | Hz, 2H), 7.87 (s, 1H), 8.15 (s, 1H), 8.25 (s, 1H), |
| | 8.99 (s, 2H), 10.80 (s, 1H), 10.96 (s, 1H). |
| | One proton missing |
| P-383 | 5% yield as off white solid |
| | (M + H)⁺ = 707.4 |
| | ¹H NMR (400 MHz, DMSO-d6): δ ppm 1.11- |
| | 1.22 (m, 2H), 1.46-1.53 (m, 3H), 1.61 (d, J = |
| | 7.2 Hz, 3H), 1.64-1.66 (m, 2H), 1.85-1.89 (m, |
| | 2H), 1.97-2.03 (m, 1H), 2.12-2.19 (m, 3H), |
| | 2.61-2.76 (m, 2H), 2.93-3.00 (m, 2H), 3.12- |
| | 3.23 (m, 4H), 3.57-3.62 (m, 2H), 3.73-3.78 |
| | (m, 1H), 3.82-3.85 (m, 2H), 4.76-4.83 (m, |
| | 1H), 6.93 (d, J = 8.0 Hz, 1H), 6.99 (d, J = 8.8 Hz, |
| | 2H), 7.13 (d, J = 8.8 Hz, 2H), 7.82 (s, 1H), 8.15 |
| | (s, 1H), 8.26 (s, 1H), 8.56 (d, J = 3.6 Hz, 1H), |
| | 8.60 (d, J = 2.4 Hz, 1H), 10.22 (brs, 1H), 10.79 |
| | (s, 1H). |
| | Two protons missing |
| P-398 | 31% yield as off white solid |
| | (M + H)⁺ = 719.4 |
| | ¹H NMR (400 MHz, DMSO-d6): δ ppm 1.11- |
| | 1.22 (m, 2H), 1.36-1.52 (m, 3H), 1.63 (d, J = |
| | 7.2 Hz, 3H), 1.66-1.68 (m, 2H), 1.87-1.89 (m, |
| | 2H), 1.98-2.03 (m, 1H), 2.12-2.18 (m, 3H), |
| | 2.44-2.46 (m, 1H), 2.61-2.68 (m, 1H), 2.71- |
| | 2.77 (m, 1H), 2.93-3.01 (m, 2H), 3.11-3.25 |
| | (m, 4H), 3.61-3.63 (m, 2H), 3.75-3.79 (m, |
| | 1H), 3.81-3.84 (m, 2H), 3.93 (s, 3H), 4.81- |
| | 4.88 (m, 1H), 6.99 (d, J = 8.4 Hz, 2H), 7.13 (d, J = |
| | 8.8 Hz, 2H), 7.25-7.34 (m, 1H), 7.41-7.43 |
| | (m, 1H), 8.18 (s, 1H), 8.24 (s, 1H), 8.49-8.51 |
| | (m, 2H), 10.79 (s, 1H), 11.12 (brs, 1H). |
| P-382 | 19% yield as off white solid |
| | [M + H]⁺ = 724.4 |
| | ¹H NMR (400 MHz, DMSO-d6): δ ppm 1.11- |
| | 1.21 (m, 2H), 1.35-1.51 (m, 3H), 1.59 (d, J = |
| | 6.8 Hz, 3H), 1.62-1.68 (m, 2H), 1.87-1.89 (m, |
| | 2H), 2.00-2.04 (m, 1H), 2.12-2.18 (m, 4H), |
| | 2.59-2.73 (m, 1H), 2.92-3.00 (m, 2H), 3.11- |
| | 3.18 (m, 2H), 3.21-3.27 (m, 3H), 3.72-3.78 |
| | (m, 1H), 3.81-3.85 (m, 2H), 4.71-4.78 (m, |
| | 1H), 6.81 (s, 1H), 6.98-7.00 (m, 2H), 7.12- |
| | 7.14 (m, 2H), 7.25 (s, 1H), 7.97-8.03 (m, 2H), |
| | 8.20-8.26 (m, 2H), 9.36-9.37 (brs, 1H), 10.78 |
| | (s, 1H). |
| | Two protons are not apparent |
| P-381 | 18% yield as off white solid |
| | (M + H)⁺ = 731.4 |
| | ¹H NMR (400 MHz, DMSO-d6): δ ppm 1.11- |
| | 1.21 (m, 2H), 1.35-1.52 (m, 3H), 1.61 (d, J = |
| | 7.2 Hz, 3H), 1.64-1.67 (m, 2H), 1.87-1.90 (m, |
| | 2H), 1.99-2.04 (m, 1H), 2.13-2.16 (m, 3H), |
| | 2.61-2.63 (m, 1H), 2.70-2.76 (m, 1H), 2.93- |
| | 3.00 (m, 2H), 3.12-3.18 (m, 2H), 3.22-3.27 |
| | (m, 2H), 3.58-3.62 (m, 3H), 3.81-3.85 (m, |
| | 2H), 4.71-4.81 (m, 1H), 6.87 (d, J = 8.0 Hz, |
| | 1H), 6.99 (d, J = 8.4 Hz, 2H), 7.13 (d, J = 8.4 Hz, |
| | 2H), 7.10-7.72 (m, 1H), 8.13 (s, 1H), 8.22 (d, J = |
| | 2.0 Hz, 1H), 8.25-8.53 (m, 2H), 10.12 (s, 1H), |
| | 10.79 (s, 1H). |
| | One proton is not apparent |
| P-379 | 29% yield a pale white solid |
| | (M + H)⁺ = 747.3 |
| | ¹H NMR (400 MHz, DMSO-d6): δ ppm 1.11- |
| | 1.22 (m, 2H), 1.30-1.41 (m, 1H), 1.43-1.53 (m, |
| | 2H), 1.61 (d, J = 6.8 Hz, 3H), 1.62-1.68 (m, |
| | 2H), 1.82-1.90 (m, 2H), 1.98-2.12 (m, 1H), |
| | 2.10-2.20 (m, 3H), 2.60-2.68 (m, 1H), 2.70- |
| | 2.78 (m, 1H), 2.88-3.12 (m, 2H), 3.11-3.19 |
| | (m, 2H), 3.22-3.29 (m, 2H), 3.59-3.62 (m, |
| | 2H), 3.76-3.81 (m, 2H), 3.82-3.89 (m, 2H), |
| | 4.68-4.88 (m, 1H), 6.94-7.00 (m, 3H), 7.13 (d, |
| | J = 8.8 Hz, 2H), 7.74 (s, 1H), 8.16 (s, 1H), 8.27 |
| | (s, 1H), 8.49 (d, J = 2.0 Hz, 1H), 8.64 (d, J = 2.0 |
| | Hz, 1H), 9.27 (s, 1H), 10.79 (s, 1H). |
| P-415 | 20% yield as white solid |
| | (M + H)⁺ = 779.4 |
| | ¹H NMR (400 MHz, DMSO-d6): δ ppm 1.13- |
| | 1.23 (m, 2H), 1.47 (s, 3H), 1.40-1.53 (m, 3H), |
| | 1.62 (d, J = 7.2 Hz, 3H), 1.65-1.67 (m, 2H), |
| | 1.89-1.92 (m, 2H), 2.00 2.10 (m, 1H), 2.14- |
| | 2.15 (m, 2H), 2.35-2.41 (m, 2H), 2.43-2.47 |
| | (m, 1H), 2.72-2.80 (m, 1H), 3.21-3.30 (m, |
| | 3H), 3.39-3.48 (m, 2H), 3.51-3.56 (m, 2H), |
| | 4.18-4.29 (m, 1H), 4.50-4.53 (m, 1H), 4.99- |
| | 5.04 (m, 1H), 7.09-7.13 (m, 1H), 7.18-7.22 |
| | (m, 1H), 7.25-7.28 (m, 1H), 7.68 (s, 1H), 7.99- |
| | 8.00 (m, 1H), 8.38 (s, 1H), 8.42 (s, 1H), 8.73 (s, |
| | 1H), 9.06-9.08 (m, 2H), 10.77 (s, 1H) |
| | Three protons are not apparent |
| P-418 | 31% yield as off white solid |
| | [M + H]⁺ = 778.7 |
| | ¹H NMR (400 MHz, DMSO-d6): δ ppm 1.17- |
| | 1.23 (m, 2H), 1.44 (s, 3H), 1.45-1.52 (m, 3H), |
| | 1.61 (d, J = 7.2 Hz, 3H), 1.65-1.67 (m, 2H), |
| | 1.87-1.91 (m, 3H), 2.05-2.18 (m, 5H), 2.32- |
| | 2.36 (m, 3H), 2.76-2.82 (m, 1H), 3.25-3.49 (m, |
| | 3H), 3.62-3.68 (m, 2H), 4.15-4.27 (m, 1H), |
| | 4.45-4.59 (m, 1H), 4.64-4.69 (m, 1H), 5.00- |
| | 5.04 (m, 1H), 6.70 (d, J = 9.2 Hz, 1H), 7.17- |
| | 7.12 (m, 1H), 7.57-7.60 (m, 1H), 7.67 (s, 1H), |
| | 7.97-7.98 (m, 1H), 8.37 (s, 1H), 8.41 (s, 1H), |
| | 8.72 (s, 1H), 9.05-9.07 (m, 2H), 10.91 (s, 1H) |
| | One proton is not apparent |
| P-419 | 17% yield as off white solid |
| | [M + H]⁺ = 769.4 |
| | ¹H NMR (400 MHz, DMSO-d6): δ ppm 1.42- |
| | 1.53 (m, 5H), 1.62 (d, J = 6.8 Hz, 3H), 1.73- |
| | 1.76 (m, 2H), 1.81-1.91 (m, 6H), 2.04-2.20 (m, |
| | 2H), 2.34-2.49 (m, 2H), 2.70-2.80 (m, 1H), |
| | 3.06-3.32 (m, 6H), 3.62-3.65 (m, 2H), 3.92- |
| | 3.95 (m, 2H), 4.99-5.10 (m, 1H), 7.21 (d, J = 8.0 |
| | Hz, 1H), 7.32 (d, J = 8.8 Hz, 1H), 7.46-7.49 (m, |
| | 1H), 7.68 (s, 1H), 8.29-8.30 (m, 1H), 8.38 (s, |
| | 1H), 8.42 (s, 1H), 8.72 (s, 1H), 9.05-9.07 (m, |
| | 2H), 10.80 (s, 1H). |
| | One proton is not apparent |
| P-426 | 19% yield as off white solid |
| | (M + H)⁺ = 769.4 |
| | ¹H NMR (400 MHz, DMSO d6): δ ppm 1.43 (s, |
| | 3H), 1.46-1.52 (m, 2H), 1.61 (d, J = 8.0 Hz, |
| | 3H), 1.72-1.91 (m, 8H), 2.03-2.17 (m, 2H), |
| | 2.32-2.40 (m, 1H), 2.73-2.81 (m, 1H), 3.04- |
| | 3.17 (m, 4H), 3.26-3.32 (m, 2H), 4.36-4.42 |
| | (m, 2H), 4.98-5.05 (m, 1H), 7.00 (d, J = 9.2 Hz, |
| | 1H), 7.20 (d, J = 8.4 Hz, 1H), 7.8 (dd, J = 2.8 Hz, |
| | and 9.2 Hz, 1H), 7.68 (s, 1H), 8.05 (d, J = 2.8 Hz, |
| | 1H), 8.37 (s, 1H), |
| | 8.42 (s, 1H), 8.72 (s, 1H), 9.05-9.07 (m, 2H), |
| | 10.92 (s, 1H). |
| | Four protons are not apparent |
| P-427 | 20% yield as off white solid |
| | [M + H]⁺ = 754.4 |
| | ¹H NMR (400 MHz, DMSO-d6): δ ppm 1.08- |
| | 1.12 (m, 2H), 1.34-1.39 (m, 6H), 1.62-1.63 (m, |
| | 3H), 1.67 (d, J = 6.8 Hz, 3H), 1.81-1.84 (m, |
| | 2H), 1.92-1.94 (m, 2H), 2.05-2.08 (m, 2H), |
| | 2.30-2.34 (m, 3H), 2.42-2.46 (m, 1H), 2.58- |
| | 2.62 (m, 1H), 3.14-3.29 (m, 3H), 3.31-3.49 |
| | (m, 2H), 3.76-3.78 (m, 1H), 4.12-4.27 (m, 1H), |
| | 4.42-4.58 (m, 1H), 4.94-4.96 (m, 1H), 6.67 (d, |
| | J = 8.8 Hz, 2H), 7.08-7.15 (m, 2H), 7.49 (s, 1H), |
| | 8.56 (d, J = 7.6 Hz, 1H), 8.71-8.73 (m, 2H), |
| | 8.76 (d, J = 7.6 Hz, 1H), 9.05 (s, 2H), 10.84 (s, |
| | 1H) |
| | One proton missing |
| P-435 | 6% yield as off white solid |
| | [M + H]⁺ = 779.2 |
| | ¹H NMR (400 MHz, DMSO-d6): δ ppm 1.18- |
| | 1.28 (m, 2H), 1.41-1.53 (m, 6H), 1.60-1.67 (m, |
| | 5H), 1.78-1.84 (m, 1H), 1.87-1.91 (m, 2H), |
| | 2.07-2.09 (m, 1H), 2.16-2.19 (m, 3H), 2.33- |
| | 2.37 (m, 3H), 2.51-2.59 (m, 1H), 2.62-2.69 (m, |
| | 1H), 2.75-2.85 (m, 1H), 3.18-3.49 (m, 3H), |
| | 3.58-3.72 (m, 2H), 4.21-4.30 (m, 1H), 4.46- |
| | 4.58 (m, 1H), 4.65-4.75 (m, 1H), 5.00-5.04 |
| | (m, 1H), 6.71-6.79 (m, 1H), 7.19-7.22 (m, 1H), |
| | 7.61-7.64 (m, 1H), 7.68 (s, 1H), 7.95-7.98 (m, |
| | 1H), 8.37 (s, 1H), 8.41 (s, 1H), 8.72 (s, 1H), 9.06- |
| | 9.07 (m, 2H), 10.93 (s, 1H) |
| P-292 | 5% yield as off white solid |
| | [M + H]⁺ = 767.7 |
| | ¹H NMR (400 MHz, DMSO-d6): δ ppm 1.39 (s, |
| | 3H), 1.48-1.53 (m, 2H), 1.61 (d, J = 7.2 Hz, |
| | 3H), 1.72-1.91 (m, 8H), 2.04-2.07 (m, 2H), |
| | 2.72-2.78 (m, 1H), 2.91-2.99 (m, 2H), 3.59- |
| | 3.63 (m, 2H), 3.84-3.87 (m, 2H), 4.45-4.48 (m, |
| | 1H), 5.00-5.06 (m, 1H), 6.95-7.02 (m, 2H), |
| | 7.16-7.21 (m, 3H), 7.67 (s, 1H), 8.37 (s, 1H), |
| | 8.41 (s, 1H), 8.72 (s, 1H), 9.05-9.07 (m, 2H), |
| | 10.86 (s, 1H) |
| | Six protons are not apparent |
| P-288 | (M + H)⁺ = 779.4 |
| | ¹H NMR (400 MHz, DMSO-d6): δ ppm 1.12- |
| | 1.22 (m, 2H), 1.47 (s, 3H), 1.50-1.53 (m, 3H), |
| | 1.62 (d, J = 7.2 Hz, 3H), 1.65-1.67 (m, 3H), |
| | 1.89-1.91 (m, 2H), 2.00-2.19 (m, 4H), 2.29- |
| | 2.33 (m, 2H), 2.40-2.45 (m, 1H), 2.72-2.80 |
| | (m, 1H), 3.09-3.29 (m, 3H), 3.37-3.46 (m, |
| | 2H), 3.51-3.58 (m, 1H), 3.63-3.72 (m, 2H), |
| | 4.20-4.31 (m, 1H), 4.50-4.53 (m, 1H), 4.99- |
| | 5.07 (m, 1H), 7.09-7.13 (m, 1H), 7.18-7.22 |
| | (m, 1H), 7.25-7.29 (m, 1H), 7.68 (s, 1H), 7.99 |
| | (s, 1H), 8.38 (s, 1H), 8.42 (s, 1H), 8.73 (s, 1H), |
| | 9.06-9.08 (m, 2H), 10.77 (s, 1H). |
| P-291 | 18% yield as off white solid |
| | (M + H)⁺ = 782.2 |
| | ¹H NMR (400 MHz, 100° C.): δ ppm 1.04-1.14 |
| | (m, 3H), 1.47 (s, 3H), 1.51-1.59 (m, 2H), 1.67 |
| | (d, J = 7.2 Hz, 3H), 1.79-1.90 (m, 4H), 1.94- |
| | 1.99 (m, 4H), 2.08-2.16 (m, 2H), 2.38-2.40 |
| | (m, 1H), 2.45-2.48 (m, 2H), 2.81-2.85 (m, |
| | 1H), 3.28-3.35 (m, 4H), 3.52-3.59 (m, 2H), |
| | 4.92-4.98 (m, 1H), 7.01-7.08 (m, 3H), 7.22- |
| | 2.75 (m, 2H), 7.75 (s, 1H), 8.32 (s, 1H), 8.47 (s, |
| | 1H), 8.66 (s, 1H), 8.97 (d, J = 2.0 Hz, 1H), 9.01 |
| | (d, J = 2.0 Hz, 1H), 10.44 (s, 1H). |
| | Three protons are not apparent |
| P-346 | 6.4% as off white solid |
| | [M + H]⁺ = 727.3 |
| | ¹H NMR (400 MHz, DMSO-d6): δ ppm 1.12- |
| | 1.21 (m, 2H), 1.35-1.39 (m, 1H), 1.46-1.53 |
| | (m, 2H), 1.61 (d, J = 6.8 Hz, 3H), 1.64-1.66 (m, |
| | 2H), 1.87-1.91 (m, 2H), 2.08-2.24 (m, 8H), |
| | 2.58-2.62 (m, 1H), 2.71-2.79 (m, 1H), 3.05- |
| | 3.26 (m, 4H), 3.61-3.64 (m, 2H), 3.75-3.79 |
| | (m, 1H), 4.41-4.55 (m, 1H), 4.99-5.03 (m, |
| | 1H), 7.21 (d, J = 8.4 Hz, 1H), 7.45-7.47 (m, |
| | 1H), 7.68 (s, 1H), 7.73 (s, 1H), 8.38 (s, 1H), 8.41 |
| | (s, 1H), 8.73 (s, 1H), 9.07-9.08 (m, 2H), 10.79 |
| | (s, 1H). |
| P-442 | 27% yield as off white solid |
| | [M + H]⁺ = 765.9 |
| | ¹H NMR (400 MHz, DMSO-d6): δ ppm 0.78 and |
| | 0.88 (t, J = 6.8 Hz, 3H), 1.13-1.22 (m, 2H), 1.33- |
| | 1.40 (m, 1H), 1.47-1.55 (m, 3H), 1.61 (d, J = |
| | 6.8 Hz, 3H), 1.65-1.78 (m, 3H), 1.87-1.90 (m, |
| | 2H), 1.98-2.04 (m, 1H), 2.15-2.18 (m, 3H), |
| | 2.59-2.63 (m, 1H), 2.72-2.79 (m, 1H), 3.01- |
| | 3.09 (m, 1H), 3.13-3.29 (m, 5H), 3.69-3.74 |
| | (m, 2H), 4.01-4.03 (m, 1H), 4.99-5.03 (m, |
| | 1H), 6.92 (d, J = 8.8 Hz, 2H), 7.10 (d, J = 8.8 Hz, |
| | 2H), 8.41 (s, 1H), 8.72 (s, 1H), 9.06-9.07 (m, |
| | 2H), 10.77 and 10.83 (s, 1H). Two protons are |
| | not apparent |
| P-448 | 7% yield as pale brown solid |
| | [M + H]⁺ = 777.9 |
| | ¹H NMR (400 MHz, DMSO-d6): δ ppm 1.11- |
| | 1.23 (m, 2H), 1.38 (s, 3H), 1.44-1.51 (m, 3H), |
| | 1.53-1.57 (m, 2H), 1.61 (d, J = 6.8 Hz, 3H), 1.85- |
| | 1.88 (m, 2H), 2.04-2.09 (m, 2H), 2.14-2.16 |
| | (m, 2H), 2.21-2.33 (m, 2H), 2.38-2.44 (m, |
| | 2H), 2.71-2.79 (m, 1H), 3.10-3.24 (m, 3H), |
| | 3.30-3.34 (m, 1H), 3.58-3.66 (m, 1H), 3.73- |
| | 3.77 (m, 1H), 3.83-3.88 (m, 4H), 4.98-5.03 |
| | (m, 1H), 6.46 (d, J = 8.8 Hz, 2H), 7.09-7.13 (m, |
| | 2H), 7.20 (d, J = 8.4 Hz, 1H), 7.68 (brs, 1H), 8.35 |
| | (s, 1H), 8.41 (brs, 1H), 8.72 (brs, 1H), 9.05 (s, |
| | 2H), 10.83 (s, 1H). |
| | Four protons are not apparent |
| P-452 | 27% yield as off white solid |
| | [M + H]⁺ = 765.9 |
| | ¹H NMR (400 MHz, DMSO-d6, VT): δ ppm 0.84- |
| | 0.87 (m, 3H), 1.19-1.26 (m, 2H), 1.39-1.56 |
| | (m, 4H), 1.63-1.71 (m, 5H), 1.89-1.92 (m, 2H), |
| | 2.09-2.19 (m, 4H), 2.65-2.71 (m, 2H), 2.79- |
| | 2.87 (m, 1H), 3.02-3.09 (m, 1H), 3.18-3.28 (m, |
| | 3H), 3.51-3.56 (m, 2H), 3.72-3.83 (m, 1H), |
| | 4.90-4.94 (m, 1H), 6.97-7.04 (m, 3H), 7.14 (d, |
| | J = 8.4 Hz, 2H), 7.72 (s, 1H), 8.27 (s, 1H), 8.43 |
| | (s, 1H), 8.63 (s, 1H), 8.94 (d, J = 2.0 Hz, 1H), |
| | 8.98 (d, J = 2.0 Hz, 1H), 10.36 (s, 1H) |
| P-453 | 11% yield as off-white solid |
| | [M + H]⁺ = 792.95 |
| | ¹H-NMR (400 MHz, DMSO-D6): δ ppm 1.09- |
| | 1.19 (m, 2H), 1.36-1.49 (m, 9H), 1.62 (d, J = 7.2 |
| | Hz, 3H), 1.79-1.95 (m, 4H), 2.04-2.07 (m, 2H), |
| | 2.11-2.19 (m, 4H), 2.27-2.39 (m, 2H), 2.43- |
| | 2.48 (m, 2H), 2.65-2.82 (m, 2H), 2.98 (m, 1H), |
| | 3.12 (m, 1H), 3.24-3.31 (m, 1H), 4.12-4.24 (m, |
| | 1H), 4.95-5.03 (m, 1H), 6.53 (d, J = 9.2 Hz, |
| | 2H), 7.04-7.08 (m, 3H), 7.35 (brs, 2H), 7.97 (s, |
| | 1H), 8.24 (s, 1H), 8.31-8.32 (m, 2H), 8.57 (s, |
| | 1H), 10.79 (s, 1H). |
| P-454 | 9% yield as off white solid |
| | [M + H]⁺ = 777.9 |
| | ¹H-NMR (400 MHz, DMSO-d6): δ ppm: 1.05- |
| | 1.29 (m, 2H), 1.34-1.52 (m, 9H), 1.61 (d, J = 6.8 |
| | Hz, 3H), 1.83-1.92 (m, 4H), 2.04-2.08 (m, 2H), |
| | 2.12-2.19 (m, 4H), 2.21-2.30 (m, 2H), 2.42- |
| | 2.48 (m, 1H), 2.68-2.84 (m, 2H), 2.98-3.05 (m, |
| | 1H), 3.13-3.15 (m, 1H), 4.15-4.19 (m, 1H), |
| | 4.99-5.03 (m, 1H), 6.53 (d, J = 9.2 Hz, 2H), 7.05 |
| | (d, J = 8.8 Hz, 2H), 7.19 (d, J = 8.4 Hz, 1H), 7.67 |
| | (s, 1H), 8.34 (s, 1H), 8.41 (s, 1H), 8.71 (s, 1H), |
| | 9.05-9.07 (m, 2H), 10.78 (s, 1H). Two protons |
| | are missing |
| P-462 | 4% yield as off white solid |
| | [M + H]⁺ = 777.9 |
| | ¹H-NMR (400 MHz, DMSO-d6): δ ppm 1.18- |
| | 1.22 (m, 2H), 1.33 (s, 3H), 1.40-1.55 (m, 5H), |
| | 1.61 (d, J = 7.2 Hz, 3H), 1.75-1.91 (m, 2H), 2.03- |
| | 2.09 (m, 4H), 2.12-2.29 (m, 4H), 2.34-2.43 |
| | (m, 2H), 2.71-2.85 (m, 2H), 2.92-3.06 (m, |
| | 2H), 3.08-3.19 (m, 2H), 4.15 (m, 1H), 4.99- |
| | 5.03 (m, 1H), 6.53 (d, J = 7.2 Hz, 2H), 7.05 (d, J = |
| | 7.2 Hz, 2H), 7.20 (d, J = 8.4 Hz, 1H), 7.67 (s, |
| | 1H), 8.35 (s, 1H), 8.41 (s, 1H), 8.72 (s, 1H), 9.05- |
| | 9.07 (m, 2H), 10.79 (s, 1H). Two protons are not |
| | apparent |
| P-414 | 16% yield |
| | [M + H]⁺ = 806.1 |
| | ¹H NMR (500 MHz, DMSO-d6) δ 9.06 (d, J = 5.7 |
| | Hz, 2H), 8.72 (s, 1H), 8.41 (s, 1H), 8.37 (s, 1H), |
| | 7.68 (s, 1H), 7.33 (br d, J = 8.6 Hz, 2H), 7.19 (br d, |
| | J = 8.3 Hz, 1H), 6.98-6.91 (m, 1H), 5.10-4.91 |
| | (m, 1H), 4.29-4.10 (m, 1H), 3.77-3.65 (m, 1H), |
| | 3.23-3.08 (m, 1H), 3.04-2.86 (m, 2H), 2.81- |
| | 2.69 (m, 1H), 2.30-2.20 (m, 1H), 2.19-2.10 (m, |
| | 4H), 2.10-2.00 (m, 1H), 2.00-1.80 (m, 6H), |
| | 1.71-1.56 (m, 9H), 1.55-1.45 (m, 2H), 1.44- |
| | 1.37 (m, 2H), 1.27-1.12 (m, 2H). |
| P-487 | 5% yield as off white solid |
| | [M + H]+ = 793.2 |
| | 1H-NMR (400 MHz, DMSO-d6): δ ppm 1.12- |
| | 1.22 (m, 2H), 1.33 (s, 3H), 1.40-1.55 (m, 6H), |
| | 1.62 (d, J = 7.2 Hz, 3H), 1.72-1.92 (m, 4H), 2.03- |
| | 2.07 (m, 2H), 2.11-2.15 (m, 2H), 2.27-2.93 |
| | (m, 2H), 2.41-2.45 (m, 1H), 2.69-2.76 (m, |
| | 1H), 2.92-3.15 (m, 2H), 4.16 (m, 1H), 4.97- |
| | 5.01 (m, 1H), 6.51-6.55 (m, 2H), 7.04-7.08 (m, |
| | 3H), 7.34 (brs, 2H), 7.97 (s, 1H), 8.24 (s, 1H), |
| | 8.31 (s, 1H), 8.32 (s, 1H), 8.57 (s, 1H), 10.79 (s, |
| | 1H) |
| | Five protons are not apparent |
| P-492 | 4% yield as off white solid |
| | [M + H]⁺= 754.4 |
| | ¹H-NMR (400 MHz, DMSO-d6): δ ppm 1.03- |
| | 1.06 (m, 2H), 1.35-1.41 (m, 9H), 1.67 (d, J = 7.2 |
| | Hz, 3H), 1.78-1.91 (m, 6H), 2.03-2.09 (m, 2H), |
| | 2.15-2.29 (m, 4H), 2.39-2.40 (m, 1H), 2.71- |
| | 2.75 (m, 1H), 2.94-2.97 (m, 1H), 3.09-3.26 (m, |
| | 3H), 3.69-3.76 (m, 1H), 4.14-4.16 (m, 1H), |
| | 4.92-4.96 (m, 1H), 6.53 (d, J = 8.8 Hz, 2H), 7.04 |
| | (d, J = 8.8 Hz, 2H), 7.48 (s, 1H), 8.53 (d, J = 7.2 |
| | Hz, 1H), 8.69-8.71 (m, 2H), 8.77 (d, J = 7.2 Hz, |
| | 1H), 9.03-9.05 (m, 2H), 10.79 (s, 1H) |

### Example S66. Preparation of (R)-2-((2-(6-((1R,4R)-2-oxa-5-azabicyclo[2.2.1]heptan-5-yl)-1H-pyrazolo[3,4-b]pyridin-1-yl)-5-(4-((1 r,4R)-4-(2-hydroxyethyl)cyclohexyl)-1H-1,2,3-triazol-1-yl)pyridin-4-yl)amino)propanenitrile (A-113)

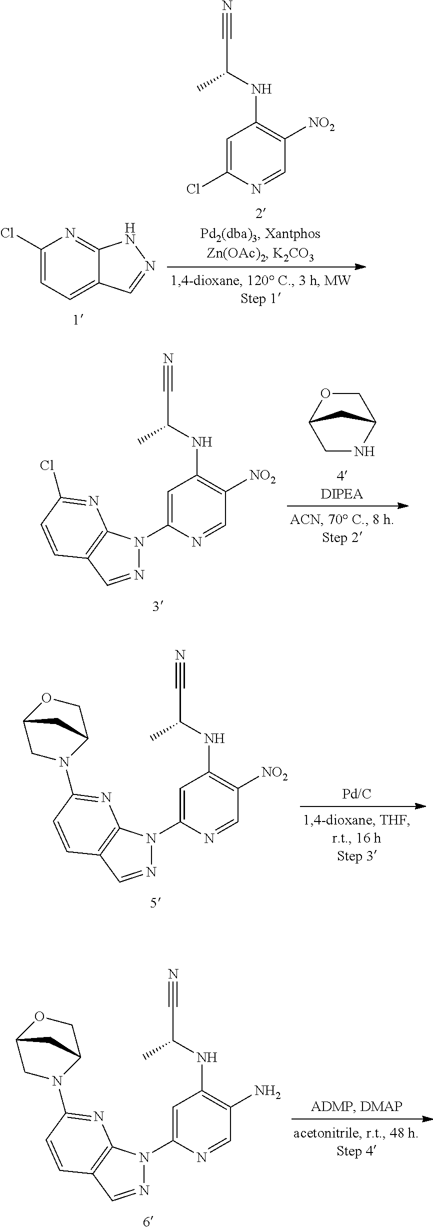 [SMILES: C[C@H](C#N)Nc1cc(Cl)ncc1[N+](=O)[O-] ; Clc1ccc2cn[nH]c2n1 ; C[C@H](C#N)Nc1cc(-n2ncc3ccc(Cl)nc32)ncc1[N+](=O)[O-] ; C1O[C@H]2CN[C@@H]1C2 ; C[C@H](C#N)Nc1cc(-n2ncc3ccc(N4C[C@H]5C[C@@H]4CO5)nc32)ncc1[N+](=O)[O-] ; C[C@H](C#N)Nc1cc(-n2ncc3ccc(N4C[C@H]5C[C@@H]4CO5)nc32)ncc1N]

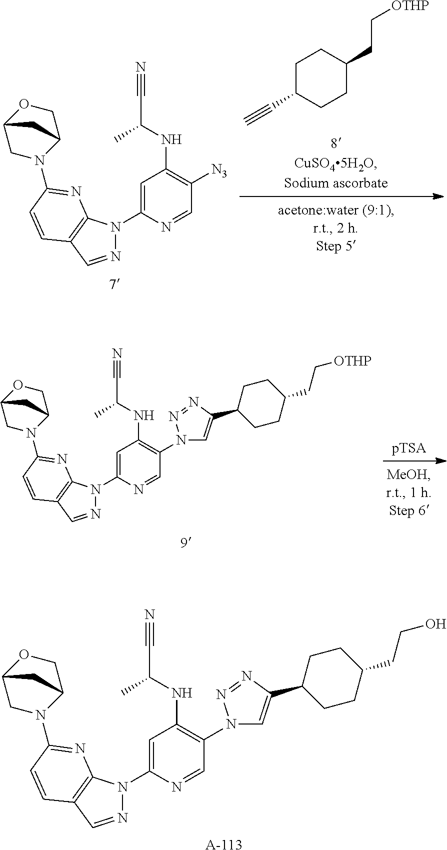

LCMS Method 1. Kinetex XB-C18, 75×3.0 mm, 2.6 Temperature: RT, Flow: 1.0 mL/min, run time: 5.0 min. Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 100% Mobile Phase B for 4.0 min and then hold for 0.6 min. MSD positive.

UPLC Method 2. Aquity BEH C18, 50×3.0 mm, 1.7 Temperature: RT, Flow: mL/min, run time: 2.6 min. Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 98% Mobile Phase B for 1.5 min and then hold for 0.5 min. MSD positive.

UPLC Method 3. Aquity BEH-C18, 50×2.1 mm, 1.7 Temperature: RT, Flow: mL/min, run time: 2.5 min. Mobile Phase Conditions: Mobile Phase-A: 0.1% TFA in H₂O, Mobile Phase-B: 0.1% TFA in ACN, Gradient: Initial 95% Mobile Phase A and 5% Mobile Phase B linear gradient to 98% Mobile Phase B for 1.5 min. MSD positive.

Step 1′. Preparation of (R)-2-((2-(6-chloro-1H-pyrazolo[3,4-b]pyridin-1-yl)-5-nitropyridin-4-yl) amino) propane nitrile 3′. To a stirred solution of 6-chloro-1H-pyrazolo[3,4-b]pyridine 1′ (1.0 g, 6.51 mmol), (R)-2-((2-chloro-5-nitropyridin-4-yl) amino)propane nitrile 2′ (2.21 g, 9.77 mmol) and potassium carbonate (2.25 g, 16.28 mmol) in 1, 4-dioxane (30 mL) was added Xantphos (0.301 g, 0.521 mmol) and zinc acetate (0.014 g, mmol) and stirred for 10 min under nitrogen atmosphere at RT. Then Pd₂(dba)₃ (1.19 g, 1.30 mmol) was added, degassed it and stirred at 120° C. for another 3 h in MW. The reaction mixture was cooled to room temperature, diluted with cold water (10 mL) and extracted with EtOAc (2×30 mL). The organic layers were collected, washed with brine solution, dried over Na₂SO₄, filtered and concentrated under reduced pressure to afford the crude product. The crude product was purified by column chromatography on silica gel with of 80% of ethyl acetate in pet ether to get (R)-2-((2-(6-chloro-1H-pyrazolo[3,4-b]pyridin-1-yl)-5-nitropyridin-4-yl)amino)propane nitrile 3′ (2.32 g, 38.3% yield). LCMS method 1: retention time: 2.03 min, [M+H]⁺=344.0.

Step 2′. Preparation of (R)-2-((2-(6-((1R,4R)-2-oxa-5-aza bicyclo[2.2.1] heptan-pyridin-1-yl)-5-nitropyridin-4-yl)amino)propanenitrile 5′. To a stirred solution of (1R,4R)-2-oxa-5-aza bicyclo[2.2.1]heptane 4′ (1.21 g, 12.2 mmol) in ACN (50 mL) was added DIPEA (3.56 mL, 20.4 mmol) and stirred for 10 minutes under nitrogen condition at RT. Then added (R)-2-((2-(6-chloro-1H-pyrazolo[3,4-b]pyridin-1-yl)-5-nitropyridin-4-yl)amino)propanenitrile 3′ (1.40 g, 4.07 mmol) and stirred at 70° C. for 8 h. The reaction mixture was cooled to room temperature, diluted with cold water (30 mL) and extracted with EtOAc (3×40 mL). The organic layers were collected, washed with brine solution, dried over Na₂SO₄, filtered and concentrated under reduced pressure to afford the crude product. The crude product was purified by column chromatography on silica gel with of 50% of ethyl acetate in pet ether to get (R)-2-((2-(6-((1R,4R)-2-oxa-5-aza bicyclo[2.2.1]heptan-5-yl)-1H-pyrazolo[3,4-b]pyridin-1-yl)-5-nitropyridin-4-yl)amino) propanenitrile 5′ (1.23 g, 58.1% yield). UPLC method 3: retention time: 0.972 min, [M+H]⁺=407.2.

Step 3′. Preparation of (R)-2-((2-(6-((1R,4R)-2-oxa-5-aza bicyclo[2.2.1] heptan-pyridin-1-yl)-5-aminopyridin-4-yl)amino)propanenitrile 6′. To a stirred solution of (R)-2-((2-(6-((1R,4R)-2-oxa-5-aza bicyclo[2.2.1] heptan-5-yl)-1H-pyrazolo[3,4-b]pyridin-1-yl)-5-nitropyridin-4-yl)amino)propanenitrile 5′ (1.1 g, 2.71 mmol) in 1,4-dioxane (45 mL) and THF (5 mL) was added palladium on carbon (0.576 g, 5.41 mmol) and stirred at 30° C. for 8 h under hydrogen atmosphere. The reaction mixture was diluted with 50 mL of 1,4-dioxane:THF (9:1) and filtered through celite bed. Then the celite bed was washed with 200 mL of MeOH:DCM (9:1) and concentrated under reduced pressure get crude product (R)-2-((2-(6-((1R,4R)-2-oxa-5-azabicyclo[2.2.1]heptan-5-yl)-1H-pyrazolo[3,4-b]pyridin-1-yl)-6′ (990 mg, 78% yield). It was used for the next step without further purification. UPLC method 2: retention time: 0.723 min, [M+H]⁺=377.2.

Step 4′. Preparation of (R)-2-((2-(6-((1R,4R)-2-oxa-5-aza bicyclo[2.2.1]heptan-5-yl)-1H-pyrazolo[3,4-b]pyridin-1-yl)-5-azidopyridin-4-yl)amino)propanenitrile 7′. To a stirred solution of (R)-2-((2-(6-((1R,4R)-2-oxa-5-aza bicyclo[2.2.1]heptan-5-yl)-1H-pyrazolo[3,4-b]pyridin-1-yl)-5-aminopyridin-4-yl) amino)propanenitrile 6′ (980 mg, 2.60 mmol) and DMAP (254 mg, 2.08 mmol) in ACN (45.0 mL) and DMF (5.0 mL) was added ADMP (2.97 g, 10.4 mmol) portion wise at RT and stirred for 48 h under nitrogen atmosphere. The reaction mixture was concentrated under reduced pressure to afford crude product. It was diluted with cold water (30 mL) and extracted with EtOAc (3×30 mL). The organic layers were collected, washed with brine solution, dried over Na₂SO₄, filtered and concentrated under reduced pressure to afford the crude product (R)-2-((2-(6-((1R,4R)-2-oxa-5-aza bicyclo[2.2.1] heptan-5-yl)-1H-pyrazolo[3,4-b]pyridin-1-yl)-5-azidopyridin-4-yl)amino)propanenitrile 7′ (810 mg). It was used for the next step without further purification. UPLC method 2: retention time: 1.023 min, [M+H]⁺=403.2.

Step 5′. Preparation of (2R)-2-((2-(6-((1R,4R)-2-oxa-5-azabicyclo[2.2.1] heptan-din-1-yl)-5-(4-((1r,4R)-4-(2-((tetrahydro-2H-pyran-2-yl)oxy)ethyl)cyclohexyl)-1H-1,2,3-triazol-1-yl)pyridin-4-yl) amino)propanenitrile 9′. To a stirred solution of (R)-2-((2-(6-((1R,4R)-2-oxa-5-azabicyclo[2.2.1]heptan-5-yl)-1H-pyrazolo[3,4-b]pyridin-1-yl)-5-azidopyridin-4-yl)amino)propanenitrile 7′ (800 mg, 1.988 mmol) and 2-(2-((1s,4s)-4-ethynylcyclohexyl) ethoxy)tetrahydro-2H-pyran 8′ (376 mg, 1.59 mmol) in acetone (27.0 mL) was added copper(II) sulfate pentahydrate (248 mg, 0.994 mmol) in water (3.0 mL) and sodium ascorbate (197 mg, 0.994 mmol) at RT and stirred at 30° C. for 2 h under nitrogen atmosphere. The reaction mixture was concentrated under reduced pressure to afford crude product. It was diluted with cold water (30 mL) and extracted with EtOAc (3×30 mL). The organic layers were collected, washed with brine solution, dried over Na₂SO₄, filtered and concentrated under reduced pressure to afford the crude product (2R)-2-((2-(6-((1R,4R)-2-oxa-azabicyclo[2.2.1] heptan-5-yl)-1H-pyrazolo[3,4-b]pyridin-1-yl)-5-(4-((1r,4R)-4-(2-((tetrahydro-2H-pyran-2-yl)oxy)ethyl)cyclohexyl)-1H-1,2,3-triazol-1-yl)pyridin-4-yl)amino)propanenitrile 9′ (2.5 g). It was used for the next step without further purification. UPLC method 2: retention time: 1.46 min, [M+H]⁺=639.2.

Step 6′. Preparation of (R)-2-((2-(6-((1R,4R)-2-oxa-5-azabicyclo[2.2.1]heptan-5-yl)-1H-pyrazolo[3,4-b]pyridin-1-yl)-5-(4-((1r,4R)-4-(2-hydroxyethyl)cyclohexyl)-1H-1,2,3-triazol-1-yl)pyridin-4-yl)amino)propanenitrile A-113. To a stirred solution of (2R)-2-((2-(6-((1R,4R)-2-oxa-5-aza bicyclo[2.2.1] heptan-5-yl)-1H-pyrazolo[3,4-b]pyridin-1-yl)-5-(4-((1r,4R)-4-(2-((tetrahydro-2H-pyran-2-yl)oxy)ethyl)cyclohexyl)-1H-1,2,3-triazol-1-yl)pyridin-4-yl)amino)propanenitrile 9′ (1.0 g, 1.57 mmol) in methanol (20.0 mL) was added p-toluenesulfonic acid monohydrate (0.238 g, 1.25 mmol) and stirred at 30° C. for 1 h. The reaction mixture was quenched with Et3N (15 drops) and concentrated under reduced pressure to afford crude product. The crude product was diluted with cold water (20 mL) and extracted with EtOAc (3×30 mL). The organic layers were collected, washed with brine solution, dried over Na₂SO₄, filtered and concentrated under reduced pressure to afford the crude product (R)-2-((2-(6-((1R,4R)-2-oxa-5-azabicyclo[2.2.1]heptan-5-yl)-1H-pyrazolo[3,4-b]pyridin-1-yl)-5-(4-((1r,4R)-4-(2-hydroxyethyl) cyclohexyl)-1H-1,2,3-triazol-1-yl)pyridin-4-yl)amino)propanenitrile A-113 (985 mg). It was used for the next step without further purification. UPLC method 2: retention time: 1.031 min, [M+H]⁺=555.2.

Table 55 summarizes the Final Compounds prepared via General Procedure X-8. All intermediates are enantiomerically pure.

TABLE 57
CBM intermediates prepared via General Procedure for CBM-6.

| Intermediate | |
|---|---|
| No. | Structure |
| C-105 | 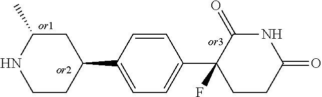 [SMILES: C[C@@H]1C[C@@H](c2ccc([C@@]3(F)CCC(=O)NC3=O)cc2)CCN1] |
| C-106a | 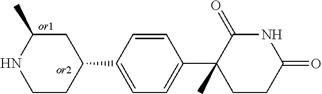 [SMILES: C[C@H]1C[C@H](c2ccc([C@@]3(C)CCC(=O)NC3=O)cc2)CCN1] |
| C-106b | 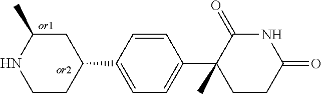 [SMILES: C[C@H]1C[C@H](c2ccc([C@@]3(C)CCC(=O)NC3=O)cc2)CCN1] |

### Example S67. Preparation of 3-(2-oxo-1-(piperidin-4-yl)-1,2-dihydropyridin-4-yl)piperidine-2,6-dione (C-107)

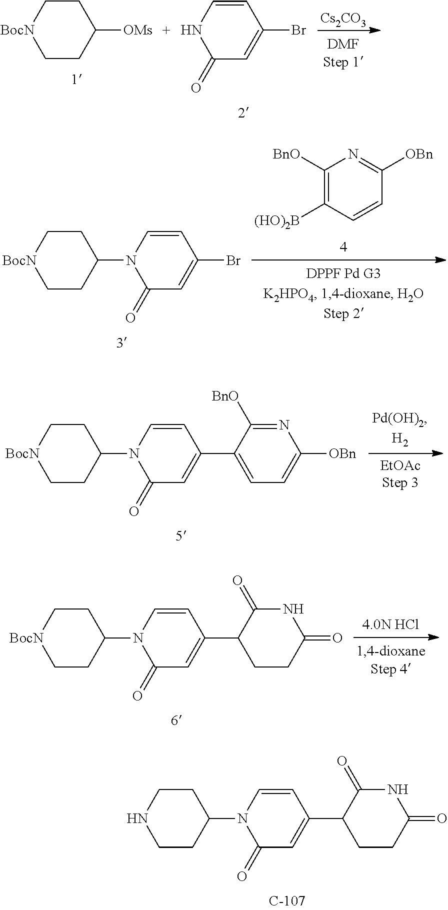

LCMS Method 1. Kinetex XB-C18, 75×3.0 mm, 2.6 Temperature: RT, Flow: 1.0 mL/min, run time: 5.0 min. Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 100% Mobile Phase B for 4.0 min and then hold for 0.6 min. MSD positive.

Step 1′. Preparation of tert-butyl 4-(4-bromo-2-oxopyridin-1(2H)-yl)piperidine-1-carboxylate 3′. To a 100 mL round bottom flask was added tert-butyl 4-((methylsulfonyl)oxy)piperidine-1-carboxylate 1′ (1541 mg, 5.52 mmol), 4-bromopyridin-2(1H)-one 2′ (800 mg, 4.60 mmol), cesium carbonate (2996 mg, 9.20 mmol) and DMF (22.989 mL). The mixture was heated to 100° C. for 2.5 days. The reaction was filtered and washed with saturated NH₄C₁ aq., extracted with EtOAc and washed with brine, dried over Na₂SO₄, filtered and concentrated. The crude compound was then purified by column chromatography using silica gel (230-400 mesh) with 50% ethyl acetate/heptane to afford the product 3′ (949 mg, 57.8%) as a white solid. LCMS method 1: retention time: 1.46 min, [M+H]⁺=357.1. ¹H NMR (400 MHz, CDCl₃) δ 7.97 (d, J=5.4 Hz, 1H), 8.02-7.91 (m, 1H), 7.03 (dd, J=5.6, 1.6 Hz, 1H), 7.06-7.00 (m, 1H), 6.95 (s, 1H), 6.95 (s, 1H), 5.23 (tt, J=7.8, 3.9 Hz, 1H), 5.32-5.12 (m, 1H), 3.88-3.70 (m, 2H), 3.31 (ddd, J=13.5, 8.7, 3.7 Hz, 1H), 3.40-3.20 (m, 1H), 2.04-1.85 (m, 2H), 1.80-1.66 (m, 2H), 1.49 (s, 9H).

Step 2′. Preparation of tert-butyl 4-(2,6-bis(benzyloxy)-2′-oxo-[3,4′-bipyridin]-r(2′H)-yl)piperidine-1-carboxylate 5′. To a 20 mL vial was added tert-butyl 4-(4-bromo-2-oxopyridin-1(2H)-yl)piperidine-1-carboxylate 3 (234 mg, 0.656 mmol), (2,6-bis(benzyloxy)pyridin-3-yl)boronic acid 4′ (200 mg, 0.597 mmol), Dioxane (2017 μl) and Water (469 μl). The mixture was bubbled with N₂ for 10 min. DPPF Pd G3 (44.8 mg, 0.048 mmol) and Potassium phosphate dibasic (312 mg, 1.79 mmol) was then added quickly and the reaction mixture was heated at 80° C. overnight. The reaction was filtered through celite. Water was removed using a pipette and the organic phase was dried over Na₂SO₄. The crude compound was then purified by column chromatography using silica gel (230-400 mesh) with 20% ethyl acetate/heptane to afford the product 5′ (250 mg, 73.8%) as a white solid. LCMS method 1: retention time: 1.60 min, [M+H]⁺=568.4.

Step 3′. Preparation of tert-butyl 4-(4-(2,6-dioxopiperidin-3-yl)-2-oxopyridin-1(2H)-yl)piperidine-1-carboxylate 6′. To a 25 mL round bottom flask was added tert-butyl 4-(2,6-bis(benzyloxy)-2′-oxo-[3,4′-bipyridin]-1′(2′H)-yl)piperidine-1-carboxylate 5′ (211 mg, mmol), Ethyl acetate (3.10 mL) and Palladium hydroxide on carbon (211 mg, 0.300 mmol). The flask was purged with N₂ then Hz. The reaction was stirred at rt overnight. The mixture was filtered through celite, concentrated and used in the next step without further purification.

Step 4′. Preparation of 3-(2-oxo-1-(piperidin-4-yl)-1,2-dihydropyridin-4-yl)piperidine-2,6-dioneC-107. To a 50 mL vial was added tert-butyl 4-(4-(2,6-dioxopiperidin-3-yl)-2-oxopyridin-1(2H)-yl)piperidine-1-carboxylate (0.072 g, 0.186 mmol), Dioxane (0.930 ml) and hydrogen chloride, 4 M in dioxane (0.930 ml, 3.72 mmol). The reaction mixture was stirred at rt for 1 h. All volatiles were then removed and the crude was used in the next step without further purification.

### Example S68. Preparation of 3-(4-(4-hydroxypiperidin-4-yl)phenyl)piperidine-2,6-dione (C-108)

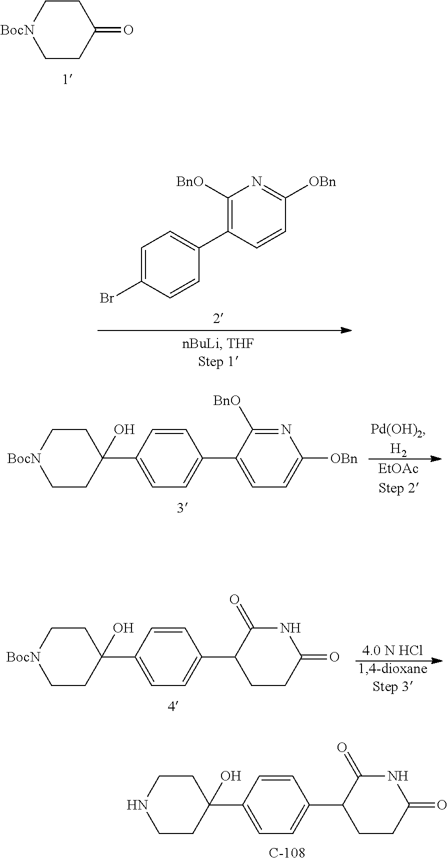 [SMILES: CC(C)(C)OC(=O)N1CCC(=O)CC1 ; Brc1ccc(-c2ccc(OCc3ccccc3)nc2OCc2ccccc2)cc1 ; CC(C)(C)OC(=O)N1CCC(O)(c2ccc(-c3ccc(OCc4ccccc4)nc3OCc3ccccc3)cc2)CC1 ; CC(C)(C)OC(=O)N1CCC(O)(c2ccc(C3CCC(=O)NC3=O)cc2)CC1 ; O=C1CCC(c2ccc(C3(O)CCNCC3)cc2)C(=O)N1]

LCMS Method 1. Kinetex XB-C18, 75×3.0 mm, 2.6 Temperature: RT, Flow: 1.0 mL/min, run time: 5.0 min. Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 100% Mobile Phase B for 4.0 min and then hold for 0.6 min. MSD positive.

Step 1′. Preparation of tert-butyl 4-(4-(2,6-bis(benzyloxy)pyridin-3-yl)phenyl)-4-hydroxypiperidine-1-carboxylate 3′. To a 50 mL round bottom flask was added 2,6-bis(benzyloxy)-3-(4-bromophenyl)pyridine 2′ (660 mg, 1.479 mmol) and THF (7393 μl). The solution was cooled to −78° C. n-Butyllithium solution in hexanes (1109 μl, 1.774 mmol) was added and the reaction was stirred at −78° C. for 1 h. tert-butyl 4-oxopiperidine-1-carboxylate (354 mg, 1.774 mmol) was then added in 0.8 mL THF and the solution was stirred at the same temperature for another 1 h. The reaction was quenched with saturated NH₄C₁ aq. and extracted with EtOAc. The organic phase was concentrated and used in the next step without further purification. LCMS method 1: retention time: 1.65 min, [M+H]⁺=567.5.

Step 2′. Preparation of tert-butyl 4-(4-(2,6-dioxopiperidin-3-yl)phenyl)-4-hydroxypiperidine-1-carboxylate 4′. To a 20 mL vial was added tert-butyl 4-(4-(2,6-bis(benzyloxy)pyridin-3-yl)phenyl)-4-hydroxypiperidine-1-carboxylate 3′ (300 mg, 0.529 mmol), Ethyl acetate (2647 μl) and Palladium hydroxide on carbon (300 mg, 0.427 mmol). The vial was purged with Na then Hz. The reaction was stirred overnight, filtered through celite, concentrated and used in the next step without further purification.

Step 3′. Preparation of 3-(4-(4-hydroxypiperidin-4-yl)phenyl)piperidine-2,6-dione C-108. To a 4 mL vial was added tert-butyl 4-(4-(2,6-dioxopiperidin-3-yl)phenyl)-4-hydroxypiperidine-1-carboxylate 4 (41 mg, 0.106 mmol), Dioxane (211 μl) and hydrogen chloride in dioxane (264 μl, 1.055 mmol). The reaction was stirred at rt for 2.5 h. All volatiles were then removed and the crude was used in the next step without further purification.

### Example S69. Preparation of 3-fluoro-3-(2-oxo-1-(piperidin-4-yl)-1,2-dihydropyridin-4-yl)piperidine-2,6-dione (C-109)

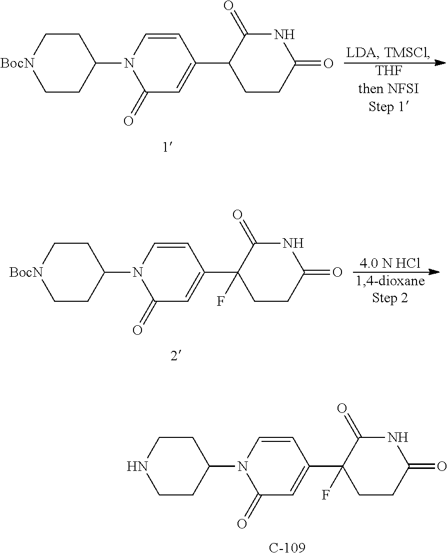 [SMILES: CC(C)(C)OC(=O)N1CCC(n2ccc(C3CCC(=O)NC3=O)cc2=O)CC1 ; CC(C)(C)OC(=O)N1CCC(n2ccc(C3(F)CCC(=O)NC3=O)cc2=O)CC1 ; O=C1CCC(F)(c2ccn(C3CCNCC3)c(=O)c2)C(=O)N1]

LCMS Method 1. Kinetex XB-C18, 75×3.0 mm, 2.6 Temperature: RT, Flow: 1.0 mL/min, run time: 5.0 min. Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 100% Mobile Phase B for 4.0 min and then hold for 0.6 min. MSD positive.

Step 1′. Preparation of tert-butyl 4-(4-(3-fluoro-2,6-dioxopiperidin-3-yl)-2-oxopyridin-1(2H)-yl)piperidine-1-carboxylate 2′. To a 10 mL round bottom flask was added tert-butyl 4-(4-(2,6-dioxopiperidin-3-yl)-2-oxopyridin-1(2H)-yl)piperidine-1-carboxylate 1′ (72.4 mg, 0.186 mmol) and THF (930 μl). The solution was cooled to −78° C. Lithium diisopropylamide solution in THF/hexanes (353 0.353 mmol) was then added and solution was stirred at the same temperature for 30 min, then at 0° C. for 30 min. Chlorotrimethylsilane (54.3 μl, 0.428 mmol) was added and the reaction was stirred for 3 h at rt. The reaction was then cooled to 0° C. NFSI (64.5 mg, 0.205 mmol) was added in one portion and the mixture was allowed to stir at rt for 40 min. The reaction was quenched with saturated NH₄C₁ aq., extracted with EtOAc, dried over Na₂SO₄, filtered, concentrated and purified by column chromatography using silica gel (230-400 mesh) with 10% methanol/DCM to afford the product 2′ (31.0 mg, as a white solid. LCMS method 1: retention time: 1.14 min, [M+H]⁺=408.3. ¹H NMR (400 MHz, DMSO-d6) δ 11.47 (s, 1H), 8.22 (d, J=5.3 Hz, 1H), 7.02 (dd, J=5.4, 1.5 Hz, 1H), 6.81 (s, 1H), 5.20 (tt, J=8.3, 3.9 Hz, 1H), 3.70 (dt, J=13.6, 4.8 Hz, 2H), 3.19 (br d, J=9.0 Hz, 2H), 2.86-2.62 (m, 2H), 2.48-2.31 (m, 2H), 1.98-1.91 (m, 2H), 1.57 (br s, 2H), 1.41 (s, 9H).

### Step 2′. Preparation of 3-fluoro-3-(2-oxo-1-(piperidin-4-yl)-1,2-dihydropyridin-4-yl)piperidine-2,6-dione C-109

To a 20 mL vial containing tert-butyl 4-(4-(3-fluoro-2,6-dioxopiperidin-3-yl)-2-oxopyridin-1(2H)-yl)piperidine-1-carboxylate 2′ (31 mg, 0.076 mmol) was added dioxane (304 IA) and 4 M hydrogen chloride in dioxane (190 IA, 0.761 mmol). The reaction was stirred at rt for 2.5 h. All volatiles were then removed and the crude was used in the next step without further purification.

### Example S70. Preparation of 3-hydroxy-3-(4-(piperidin-4-yl)phenyl)piperidine-2,6-dione (C-110)

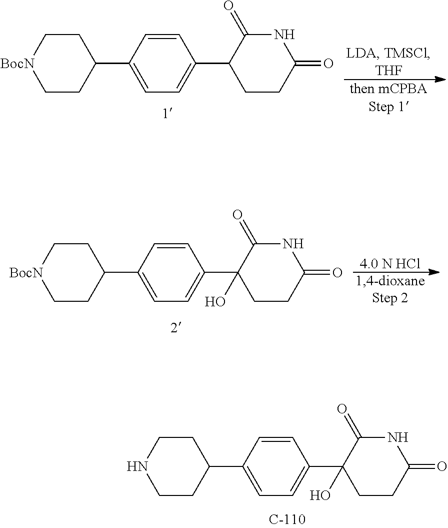 [SMILES: CC(C)(C)OC(=O)N1CCC(c2ccc(C3CCC(=O)NC3=O)cc2)CC1 ; CC(C)(C)OC(=O)N1CCC(c2ccc(C3(O)CCC(=O)NC3=O)cc2)CC1 ; O=C1CCC(O)(c2ccc(C3CCNCC3)cc2)C(=O)N1]

LCMS Method 1. Kinetex XB-C18, 75×3.0 mm, 2.6 Temperature: RT, Flow: 1.0 mL/min, run time: 5.0 min. Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 100% Mobile Phase B for 4.0 min and then hold for 0.6 min. MSD positive.

Step 1′. Preparation of tert-butyl 4-(4-(3-hydroxy-2,6-dioxopiperidin-3-yl)phenyl)piperidine-1-carboxylate 2′. To a 10 mL round bottom flask was added tert-butyl 4-(4-(2,6-dioxopiperidin-3-yl)phenyl)piperidine-1-carboxylate 1′ (100 mg, 0.268 mmol) and THF (1342 μl). The solution was cooled to −78° C. before the addition of lithium diisopropylamide solution in THF/hexanes (524 0.524 mmol). The resulting yellow solution was stirred at −78° C. for 20 min then at 0° C. for 30 min. TMSCl (7811.1, 0.618 mmol) was then added and the reaction was stirred at rt for 2.5 h. The reaction was cooled back down to −78° C. and 3-chloroperoxybenzoic acid (78 mg, 0.349 mmol) was added as a solid. The white slurry was stirred at this temperature for 1 h. The reaction mixture was then quenched with NaHSO₃ solid and saturated NH₄C₁ aq. The organic phase was collected and concentrated. The resulting oil was purified by column chromatography using silica gel (230-400 mesh) with 10% methanol/DCM to afford the desired product 2′ (7 mg, 6.7%) as a purple liquid. LCMS method 1: retention time: 1.12 min, [M−tBu+H]=333.3. ¹H NMR (400 MHz, DMSO-d6) δ 7.25-7.20 (m, 2H), 7.18-7.13 (m, 2H), 4.06 (br s, 2H), 2.90-2.77 (m, 3H), 2.73-2.62 (m, 2H), 2.22-2.10 (m, 1H), 2.07-1.98 (m, 3H), 1.82-1.68 (m, 2H), 1.55-1.46 (m, 2H), 1.42 (s, 9H).

Step 2′. Preparation of 3-hydroxy-3-(4-(piperidin-4-yl)phenyl)piperidine-2,6-dione C-110. To a 20 mL vial containing tert-butyl 4-(4-(3-hydroxy-2,6-dioxopiperidin-3-yl)phenyl)piperidine-1-carboxylate 2′ (7 mg, 0.018 mmol) was added dioxane (360 IA) and 4 M hydrogen chloride in dioxane (90 0.360 mmol). The reaction was stirred at rt for 1 h. All volatiles were then removed and the crude was used in the next step without further purfications.

### Example S71. Preparation of (3R)-3-fluoro-3-(4-(3-methylpiperidin-4-yl)phenyl)piperidine-2,6-dione (C-111d)

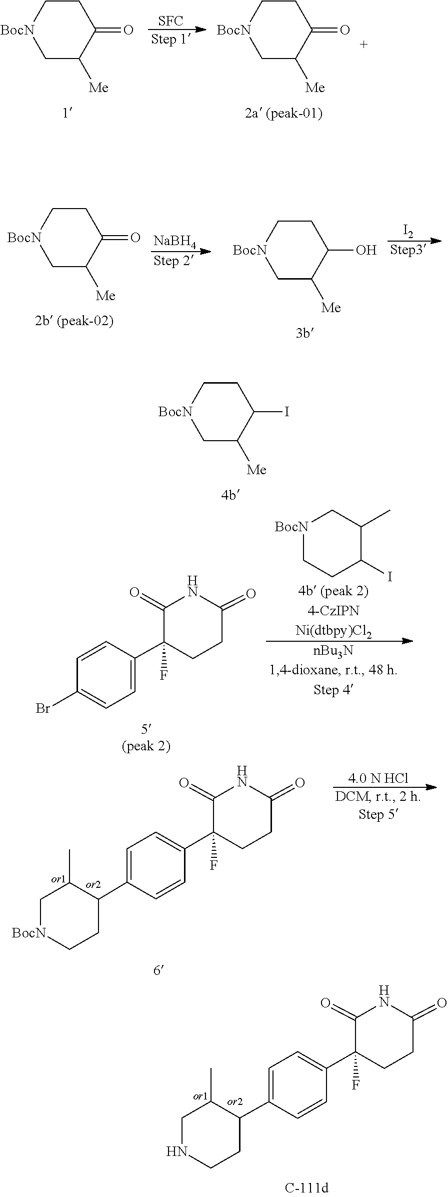

LCMS Method 1. Kinetex XB-C18, 75×3.0 mm, 2.6 Temperature: RT, Flow: 1.0 mL/min, run time: 5.0 min. Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 98% Mobile Phase A and 2% Mobile Phase B linear gradient to 100% Mobile Phase B for 4 min. MSD positive.

LCmS Method 2. X-Bridge C18, 50×4.6 mm, 5 Temperature: RT, Flow: 1.0 mL/min, run time: 5.5 min. Mobile Phase Conditions: Mobile Phase-A: 0.1% TFA in H₂O, Mobile Phase-B: 0.1% TFA in CAN, Gradient: Initial 95% Mobile Phase A and 5% Mobile Phase B linear gradient to 95% Mobile Phase B for 2.5 min. MSD positive.

Step 1′. Chiral SFC separation of tert-butyl 3-methyl-4-oxopiperidine-1-carboxylate 2a′ and 2b′. tert-butyl 3-methyl-4-oxopiperidine-1-carboxylate 1′ (25.0 g) was separated by the SFC to get tert-butyl 3-methyl-4-oxopiperidine-1-carboxylate Peak-1 2a′ (11.0 g, RT 2.12 min) and tert-butyl 3-methyl-4-oxopiperidine-1-carboxylate Peak-2 2b′ (11.0 g, RT 2.23 min). SFC Method: Chiralpak-IG, 250×4.6 mm, 5.0 Flow: 3.0 mL/min. Co-Solvent: methanol. tert-butyl 3-methyl-4-oxopiperidine-1-carboxylate Peak-1 2a′ (11.0 g, RT 2.12 min, ee 95.0%). tert-butyl 3-methyl-4-oxopiperidine-1-carboxylate Peak-2 2b′ (11.0 g, RT 2.23 min, ee 98.0%).

Step 2′. Preparation of tert-butyl 4-hydroxy-3-methylpiperidine-1-carboxylate 313′. To a cooled, well-stirred solution of tert-butyl 3-methyl-4-oxopiperidine-1-carboxylate Peak-2 2b′ (1.5 g, 7.03 mmol, 1.0 eq.) in methanol (10.0 mL) at 0° C. was added sodium borohydride (0.532 g, 14.1 mmol, 2.0 eq.) portion wise. The reaction mixture was stirred for 2 h at 25-30° C. The solvent was completely under vacuum, diluted the residue with water (10 mL), and extracted with ethyl acetate (3×15 mL). Combined all organic layers, washed with brine (20 mL), dried over sodium sulphate, and concentrated under reduced pressure to get crude compound. The crude compound was purified by column chromatography using silica gel (230-400 mesh) with 50% ethyl acetate/pet ether to obtain tertbutyl 4-hydroxy-3-methylpiperidine-1-carboxylate 3b′ (1.1 g, 5.11 mmol, 72.6% yield). LCMS method 1: retention time: 1.66 min, 99.51% purity at 220 nm, [M+H−100]⁺=116.2.

Step 3′. Preparation of tert-butyl 4-iodo-3-methylpiperidine-1-carboxylate 4b′. To a well-stirred solution of tert-butyl 4-hydroxy-3-methylpiperidine-1-carboxylate 3b′ (1.0 g, 4.64 mmol, 1.0 eq.) in THF (5.0 mL) was added iodine (2.358 g, 9.29 mmol, 1.0 eq.) followed by imidazole (0.632 g, 9.29 mmol, 1.0 eq.) and then triphenylphosphine (2.437 g, 9.29 mmol, 1.0 eq.) into the reaction mass. Stirred the reaction mass for 16 h at 25-30° C. It was treated with water (5.0 mL) and extracted with ethyl acetate(3×15 mL). Combined all organic layers, washed with brine (20 mL), dried over sodium sulphate, and distilled the solvent completely under reduced pressure to get crude compound. The crude compound was purified by column chromatography using silica gel (230-400 mesh) with 30% ethyl acetate/pet ether to obtain 4-iodo-3-methylpiperidine 4b′ (950 mg, 4.05 mmol, 87% yield). LCMS method 2: retention time: 3.17 min, 96% purity at 220 nm, [M+H]⁺=226.0.

Step 4′. Preparation of tert-butyl (R)-4-(4-(5-(tert-butoxy)-2-cyano-5-oxopentan-2-yl)phenyl)-3,6-dihydropyridine-1(2H)-carboxylate 6′. To a stirred solution of (S)-3-(4-bromophenyl)-3-fluoropiperidine-2,6-dione (300 mg, 0.88 mmol, 1.0 eq.) 5′ and tert-butyl 4-iodo-3-methylpiperidine-1-carboxylate 4b′ (317 mg, 1.06 mmol, 1.2 eq.) in 1,4-dioxane (6 mL) was added tributylamine (492 mg, 2.658 mmol, 3.0 eq.). The reaction mixture was purged with nitrogen for 10 min followed by the addition of 4-CzIPN (13.98 mg, 0.018 mmol, 0.02 eq.) and Ni(dtbpy)Cl₂ (32.25 mg, 0.09 mmol, 0.1 eq.). The reaction mixture was irradiated under blue LED and stirred at room temperature for 48 h. The reaction mixture was concentrated under vacuum to get the crude compound. The crude compound was purified by reverse phase column chromatography to obtain tert-butyl 4-(4-((R)-3-fluoro-2,6-dioxopiperidin-3-yl) phenyl)-3-methylpiperidine-1-carboxylate 6′ (105 mg, 9.0% yield) as beige solid. LCMS method 1: retention time: 2.630 min, 89.4% purity at 220 nm, [(M+H)-100]⁺=307.3.

Step 5′. Preparation of (3R)-3-fluoro-3-(4-(3-methylpiperidin-4-yl)phenyl)piperidine-2,6-dione·2HCl C-111d. To a stirred solution of tert-butyl 4-(4-((R)-3-fluoro-2,6-dioxopiperidin-3-yl) phenyl)-3-methylpiperidine-1-carboxylate 6′ (100 mg, 0.247 mmol, 1.0 eq.) in DCM (3.0 mL) at 0° C. was added 4.0 N HCl in 1,4-dioxane (0.309 mL, 1.236 mmol, 5.0 eq.). The resulting reaction mixture was stirred at room temperature for 2 h. The reaction mixture was concentrated under reduced pressure to obtain crude (3R)-3-fluoro-3-(4-(3-methylpiperidin-4-yl) phenyl)piperidine-2,6-dione·2HCl C-111d (90 mg, 0.175 mmol, 70.7% yield) as an off-white solid. It was used for the next step without further purification.

LCMS method 1: retention time: 1.910 min, [M+H]⁺=305.1.

Table 58 summarizes the intermediates that were synthesized using a procedure similar to Example S71.

TABLE 58
Intermediate Compounds Prepared via a Procedure Similar to Example S71

| Intermediate No. | Structure |
|---|---|
| C-111a (peak 1 of fluoroglutarimide separation, peak 1 of 4-piperidone separation) | 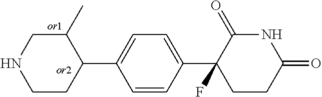 [SMILES: CC1CNCCC1c1ccc([C@@]2(F)CCC(=O)NC2=O)cc1] |
| C-111b (peak 1 of fluoroglutarimide separation, peak 2 of 4-piperidone separation) | 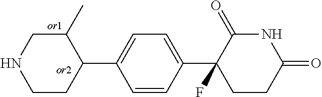 [SMILES: CC1CNCCC1c1ccc([C@@]2(F)CCC(=O)NC2=O)cc1] |
| C-111c (peak 2 of fluoroglutarimide separation, peak 1 of 4-piperidone separation) | 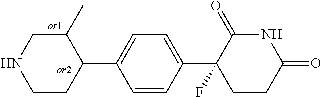 [SMILES: CC1CNCCC1c1ccc([C@]2(F)CCC(=O)NC2=O)cc1] |
| C-112a (peak 2 of 4-piperidone separation) | 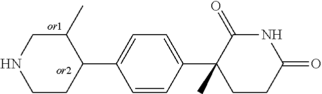 [SMILES: CC1CNCCC1c1ccc([C@@]2(C)CCC(=O)NC2=O)cc1] |
| C-112b (peak 1 of 4-piperidone separation) | 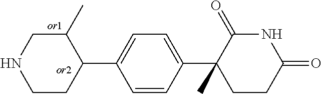 [SMILES: CC1CNCCC1c1ccc([C@@]2(C)CCC(=O)NC2=O)cc1] |

### Example S72. Preparation of (3R)-3-(4-(3-fluoropiperidin-4-yl)phenyl)-3-methylpiperidine-2,6-dione (C-113a)

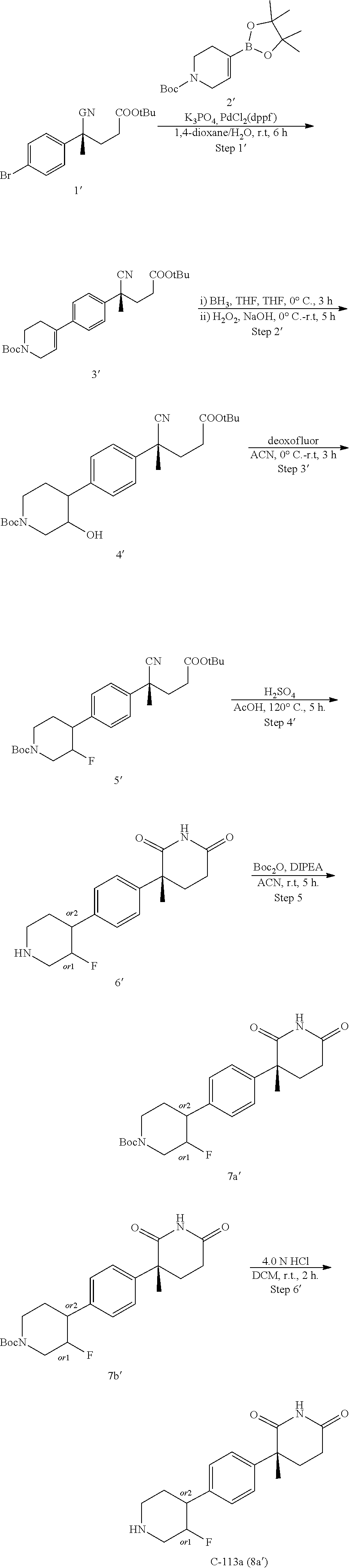

UPLC Method 1. Aquity BEH-C18, 50×3.0 mm, 1.7 Temperature: RT, Flow: mL/min, run time: 2.5 min. Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 98% Mobile Phase B for 1.5 min. MSD positive.

UPLC Method 2. Aquity BEH-C18, 50×3.0 mm, 1.7 μm. Temperature: RT, Flow: mL/min, run time: 2.5 min. Mobile Phase Conditions: Mobile Phase-A: 0.1% TFA in H₂O, Mobile Phase-B: 0.1% TFA in CAN, Gradient: Initial 80% Mobile Phase A and 20% Mobile Phase B linear gradient to 98% Mobile Phase B for 1.5 min. MSD positive.

LCMS Method 3. Kinetex XB-C18, 75×3.0 mm, 2.6 μm. Temperature: RT, Flow: 1.0 mL/min, run time: 5.0 min. Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 98% Mobile Phase A and 2% Mobile Phase B linear gradient to 100% Mobile Phase B for 4 min. MSD positive.

Step 1′. Preparation of tert-butyl (R)-4-(4-(5-(tert-butoxy)-2-cyano-5-oxopentan-2-yl)phenyl)-3,6-dihydropyridine-1(2H)-carboxylate 3′. To a stirred solution of tert-butyl (R)-4-(4-bromophenyl)-4-cyanopentanoate 1′ (1 g, 2.96 mmol, 1.0 eq) in a mixture of 1,4-dioxane/water (5:1) (24 mL) was added tert-butyl 4-(4,4,5,5-tetramethyl-1,3-dioxolan-2-yl)-3,6-dihydropyridine-1(2H)-carboxylate 2′ (0.921 g, 2.96 mmol, 1.0 eq) followed by potassium phosphate dibasic (1.030 g, 5.91 mmol, 2.0 eq). The resulting reaction mixture was degassed with Na for 5 min, then PdCl₂(dppf) (0.108 g, 0.148 mmol, 0.05 eq) was added. The reaction mixture was stirred at r.t for 6 h. The reaction mixture was treated with ice-cold water and extracted with EtOAc (2×150 mL). The combined organics were dried over Na₂SO₄ and concentrated under reduced pressure to obtain the crude compound. The crude compound was purified by column chromatography using silica gel (230-400 mesh) with 10% ethyl acetate/pet ether to obtain tert-butyl (R)-4-(4-(5-(tertbutoxy)-2-cyano-5-oxopentan-2-yl)phenyl)-3,6-dihydropyridine-1(2H)-carboxylate 3′ (1.3 g, 2.66 mmol, 90% yield) as a pale yellow gum. UPLC method 1: retention time: 1.866 min, 90.8% purity at 220 nm, [(M+H)-100]⁺=341.2.

Step 2′. Preparation of tert-butyl 4-(4-((R)-5-(tert-butoxy)-2-cyano-5-oxopentan-2-yl)phenyl)-3-hydroxypiperidine-1-carboxylate 4′. To a cooled stirred solution of tert-butyl (R)-4-(4-(5-(tert-butoxy)-2-cyano-5-oxopentan-2-yl)phenyl)-3,6-dihydropyridine-1(2H)-carboxylate 3′ (3.8 g, 18.09 mmol, 1.0 eq) in THF (5 mL) was added BH₃·THF (3.75 mL, 3.75 mmol, 1.5 eq) at 0° C. dropwise. The resulting reaction was slowly warmed to r.t and stirred for 3 h. The reaction mixture was cooled to 0° C. and quenched with 10% NaOH (20 ml) slowly, followed by H₂O₂ (1.275 mL, 12.48 mmol, 2.0 eq). The reaction mixture was slowly warmed to r.t and stirred for 5 h. The reaction mixture was treated with ice-cold water and extracted with EtOAc (2×150 mL). The combined organics were dried over Na₂SO₄ and concentrated under reduced pressure to obtain the crude compound tert-butyl 4-(4-(((R)-5-(tert-butoxy)-2-cyano-5-oxopentan-2-yl)phenyl)-3-hydroxypiperidine-1-carboxylate 4′ (800 mg, 1.169 mmol, 46.8% yield) as a colorless liquid. It was used for the next step without further purification. UPLC method 1: retention time: 1.643 min, [(M+H)-100]⁺=359.2.

Step 3′. Preparation of tert-butyl 4-(4-(((R)-5-(tert-butoxy)-2-cyano-5-oxopentan-2-yl)phenyl)-3-fluoropiperidine-1-carboxylate 5′. To a stirred solution of tert-butyl 4-(4-((R)-5-(tert-butoxy)-2-cyano-5-oxopentan-2-yl)phenyl)-3-hydroxypiperidine-1-carboxylate 4′ (800 mg, 1.744 mmol, 1.0 eq) in acetonitrile (10.0 mL) was added deoxofluor 50% in toluene (1.930 mL, 5.23 mmol, 3.0 eq) at 0° C. The reaction mixture was stirred at the same temperature for 30 min, slowly warmed to r.t, and stirred for 3 h. The reaction mixture was quenched with sat NH₄C₁ solution and extracted with EtOAc (2×100 mL). The combined organics were dried over Na₂SO₄ and concentrated under reduced pressure to obtain the crude compound. The crude compound was purified by column chromatography using silica gel (230-400 mesh) with 15% ethyl acetate/pet ether to obtain tert-butyl 4-(4-((R)-5-(tert-butoxy)-2-cyano-5-oxopentan-2-yl)phenyl)-3-fluoropiperidine-1-carboxylate 5′ (510 mg, 0.786 mmol, yield) as a colorless oil. LCMS method 3: retention time: 2.811 min, [(M+H)-100]⁺=361.2.

Step 4′. Preparation of (3R)-3-(4-(3-fluoropiperidin-4-yl)phenyl)-3-methylpiperidine-2,6-dione 6′. To a stirred solution of tert-butyl 4-(4-(((R)-5-(tert-butoxy)-2-cyano-5-oxopentan-2-yl)phenyl)-3-fluoropiperidine-1-carboxylate 5′ (320 mg, 0.695 mmol, 1.0 eq) in AcOH (2.0 mL) was added H₂SO₄ (0.111 mL, 2.084 mmol, 3.0 eq.). The resulting reaction mixture was heated to 120° C. and stirred for 3 h. The reaction mixture was concentrated under reduced pressure to obtain (3R)-3-(4-(3-fluoropiperidin-4-yl)phenyl)-3-methylpiperidine-2,6-dione, sulfuric acid salt 6′ (280 mg) as a brown color gum. It was used for the next step without further purification. UPLC method 2: retention time: 0.590 min, 92.04% purity at 220 nm [M+H]⁺=305.2.

Step 5′. Preparation of tert-butyl 3-fluoro-4-(4-(((R)-3-methyl-2,6-dioxopiperidin-3-yl)phenyl)piperidine-1-carboxylate 7a′ and tert-butyl 3-fluoro-4-(4-((R)-3-methyl-2,6-dioxopiperidin-3-yl)phenyl)piperidine-1-carboxylate 7b″. To a stirred solution of (3R)-3-(4-(3-fluoropiperidin-4-yl)phenyl)-3-methylpiperidine-2,6-dione 6′ (280 mg, 0.920 mmol, 1.0 eq.) in Acetonitrile (5 mL) at r.t was added DIPEA (0.256 mL, 1.104 mmol, 5.0 eq.). Then Di-tert-butyl decarbonate (1.941 mL, 8.36 mmol, 1.2 eq.) was added to the reaction mixture, and the resulting reaction mixture was stirred at RT for 5 h. The reaction mixture was concentrated under reduced pressure to obtain the crude product. The crude product was purified by column chromatography using silica gel (230-400 mesh) with 30% ethyl acetate/pet ether to obtain the diastereomeric mixture of tert-butyl 3-fluoro-4-(4-((R)-3-methyl-2,6-dioxopiperidin-3-yl)phenyl)piperidine-1-carboxylate 7′ (300 mg, 0.742 mmol). 300 mg of the material was purified by chiral SFC to obtain Peak-1 7a′ (100 mg, 0.232 mmol) and Peak 2 7b′ (110 mg, mmol). SFC Method: Cellulose-4, 250×4.6 mm, 5.0 Flow: 3.0 mL/min. Co-Solvent: IPA. tert-butyl 3-fluoro-4-(4-((R)-3-methyl-2,6-dioxopiperidin-3-yl)phenyl)piperidine-1-carboxylate 7a′: RT 5.067; ee 100%; ¹H NMR (400 MHz, DMSO): δ ppm 1.43 (s, 3H), 1.44 (s, 9H), 1.61-1.64 (m, 1H), 1.78-1.81 (m, 1H), 2.04-2.10 (m, 2H), 2.35-2.38 (m, 1H), 2.41-2.48 (m, 1H), 2.72-2.91 (m, 3H), 3.93-3.96 (m, 1H), 4.25-4.31 (m, 1H), 4.56-4.72 (m, 1H), 7.25 (d, J=8.4 Hz, 2H), 7.35 (d, J=8.4 Hz, 2H), 10.90 (s, 1H). tert-butyl 3-fluoro-4-(4-((R)-3-methyl-2,6-dioxopiperidin-3-yl)phenyl)piperidine-1-carboxylate 713′: RT 6.112; ee 100%; ¹H NMR (400 MHz, DMSO): δ ppm 1.43 (s, 3H), 1.44 (s, 9H), 1.59-1.63 (m, 1H), 1.78-1.82 (m, 1H), 2.04-2.10 (m, 2H), 2.35-2.39 (m, 1H), 2.43-2.48 (m, 1H), 2.72-2.91 (m, 3H), 3.93-3.96 (m, 1H), 4.25-4.31 (m, 1H), 4.56-4.72 (m, 1H), 7.25 (d, J=8.4 Hz, 2H), 7.35 (d, J=8.4 Hz, 2H), 10.90 (s, 1H).

Step 6′. Preparation of (3R)-3-(4-(3-fluoropiperidin-4-yl)phenyl)-3-methylpiperidine-2,6-dione C-113a. To a stirred solution of tert-butyl 3-fluoro-4-(4-((R)-3-methyl-2,6-dioxopiperidin-3-yl)phenyl)piperidine-1-carboxylate 7a′ (90 mg, 0.223 mmol, 1.0 eq.) in DCM (5 mL, 0.903 M) at r.t was added 4.0 N HCl in 1,4-dioxane (0.278 mL, 1.113 mmol, 5.0 eq.). The resulting reaction mixture was stirred at RT for 3 h. The reaction mixture was concentrated under reduced pressure to obtain crude (3R)-3-(4-(3-fluoropiperidin-4-yl)phenyl)-3-methylpiperidine-2,6-dione C-113a (80 mg). It was used for the next step without further purification. LCMS method 3: retention time: 1.852 min, 98.68% purity at 220 nm, [M+H]⁺=305.2.

Table 60 summarizes the intermediate synthesized using a procedure similar to Example S72.

TABLE 60
Intermediate Compound Prepared via a Procedure Similar to Example S72,

| Intermediate No. | Structure |
|---|---|
| C-113b (peak 2 of fluoropiperidine separation) | 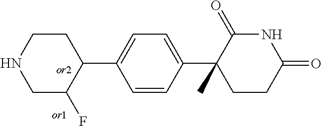 [SMILES: C[C@]1(c2ccc(C3CCNCC3F)cc2)CCC(=O)NC1=O] |

### Example S73. Preparation of (R)-3-(4-(4-fluoropiperidin-4-yl)phenyl)-3-methylpiperidine-2,6-dione (C-114)

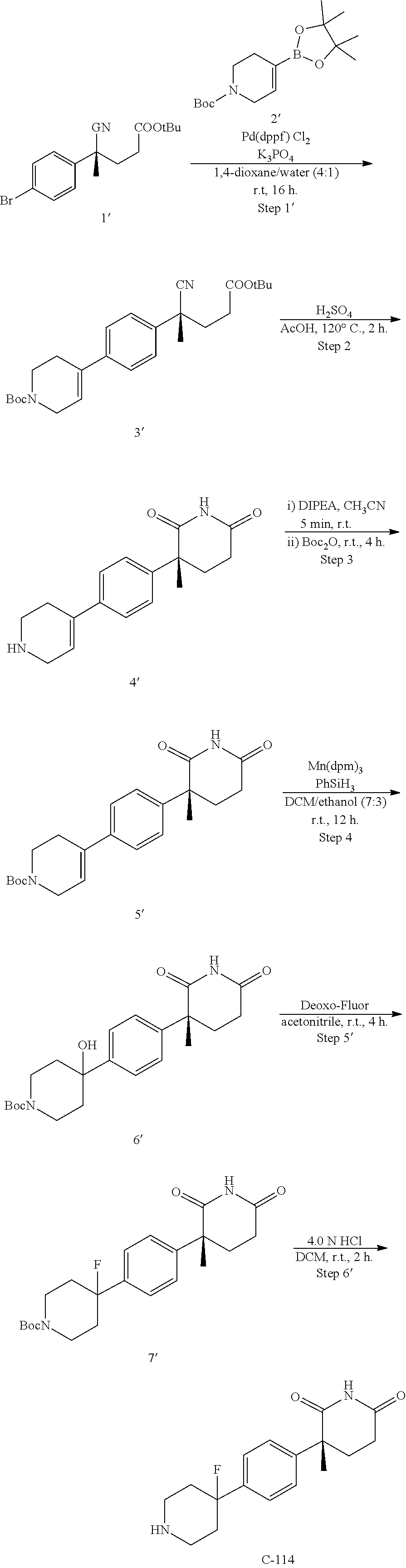

LCMS Method 1. X-Bridge-C18, 50×4.6 mm, 5.0 Temperature: RT, Flow: 1.0 mL/min, run time: 5.5 min. Mobile Phase Conditions: Mobile Phase-A: 0.1% TFA in H₂O, Mobile Phase-B: 0.1% TFA in ACN, Gradient: Initial 95% Mobile Phase A and 5% Mobile Phase B linear gradient to 95% Mobile Phase B for 2.5 min. MSD positive.

UPLC Method 2. Aquity BEH-C18, 50×2.1 mm, 1.7 Temperature: RT, Flow: mL/min, run time: 2.0 min. Mobile Phase Conditions: Mobile Phase-A: 0.1% TFA in H₂O, Mobile Phase-B: 0.1% TFA in ACN, Gradient: Initial 80% Mobile Phase A and 20% Mobile Phase B linear gradient to 98% Mobile Phase B for 1.5 min. MSD positive.

LCMS Method 3. Kinetex XB-C18, 75×3.0 mm, 2.6 Temperature: RT, Flow: 1.0 mL/min, run time: 5.0 min. Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 98% Mobile Phase A and 2% Mobile Phase B linear gradient to 100% Mobile Phase B for 4 min. MSD positive.

Step 1′. Preparation of tert-butyl (R)-4-(4-(5-(tert-butoxy)-2-cyano-5-oxopentan-2-yl)phenyl)-3,6-dihydropyridine-1(2H)-carboxylate 3′. To a stirred solution of tert-butyl (R)-4-(4-bromophenyl)-4-cyanopentanoate 1′ (5.0 g, 14.78 mmol) and tert-butyl 4-(4,4,5,5-tetramethyl-1,3,2-dioxaborolan-2-yl)-3,6-dihydropyridine-1(2H)-carboxylate 2′ (4.11 g, 13.30 mmol) in 1,4-dioxane (40 mL) was added K₃PO₄ (7.83 g, 37.0 mmol) and water (10 mL). The reaction mixture was purged with nitrogen for 15 min followed by Pd(dppf)C₁₂ (0.541 g, 0.739 mmol) was added. The reaction mixture was stirred at room temperature for 16 h. The reaction mixture was diluted with water (200 mL) and extracted using ethyl acetate (3×50 mL). The combined organic extracts were washed with brine (2×50 mL), dried over sodium sulphate, filtered, and concentrated under reduced pressure to obtain the crude compound. The crude compound was purified by column chromatography using silica gel (230-400 mesh) with 15% ethyl acetate/pet ether to obtain tert-butyl (R)-4-(4-(5-(tert-butoxy)-2-cyano-5-oxopentan-2-yl)phenyl)-3,6-dihydropyridine-1(2H)-carboxylate 3′ (5.8 g, 69.4% yield) as a colorless semi-solid. LCMS method 1: retention time: 3.060 min, [(M+H)−Boc−56]⁺=285.2.

Step 2′. Preparation of (R)-3-methyl-3-(4-(1,2,3,6-tetrahydropyridin-4-yl)phenyl)piperidine-2,6-dione 4′. To a stirred solution of tert-butyl (R)-4-(4-(5-(tert-butoxy)-2-cyano-5-oxopentan-2-yl)phenyl)-3,6-dihydropyridine-1(2H)-carboxylate 3′ (2.6 g, 4.60 mmol) in AcOH (15.0 mL) was added H₂SO₄ (0.493 mL, 9.21 mmol). The resulting reaction mixture was heated to 120° C. and stirred for 2 h. The reaction mixture was concentrated under reduced pressure to obtain (R)-3-methyl-3-(4-(1,2,3,6-tetrahydropyridin-4-yl)phenyl)piperidine-2,6-dione 4′ (1.41 g, 94% yield) as a brown color gum. It was used for the next step without further purification. UPLC method 2: retention time: 0.579 min, [M+H]⁺=285.2.

Step 3′. Preparation of tert-butyl (R)-4-(4-(3-methyl-2,6-dioxopiperidin-3-yl)phenyl)-3,6-dihydropyridine-1(2H)-carboxylate 5′. To a stirred solution of (R)-3-methyl-3-(4-(1,2,3,6-tetrahydropyridin-4-yl)phenyl)piperidine-2,6-dione 4′ (1.41 g, 4.31 mmol) in acetonitrile (20 mL) at 0° C. was added DIPEA (3.76 mL, 21.57 mmol) and stirred for 5 min. Then di-tert-butyl dicarbonate (1.487 mL, 6.47 mmol) was added to the reaction mixture, and the resulting reaction mixture was stirred at room temperature for 12 h. The crude product was quenched with cold water (20 mL) and extracted with ethyl acetate (2×400 mL). The organic layer was washed with brine (2×30 mL), dried over sodium sulphate, filtered, and concentrated under reduced pressure to obtain the crude tert-butyl (R)-4-(4-(3-methyl-2,6-dioxopiperidin-3-yl)phenyl)-3,6-dihydropyridine-1(2H)-carboxylate 5′ (1.39 g, 77% yield) as a light yellow solid. LCMS method 3: retention time: 2.807 min, 92.3% purity at 220 nm, [(M+H)-Boc]⁺=285.2. ¹H NMR (400 MHz, DMSO d6): δ ppm 1.42-1.43 (m, 12H), 2.07-2.12 (m, 2H), 2.42-2.48 (m, 4H), 3.31-3.54 (t, J=5.6 Hz, 2H), 3.99 (brs, 2H), 6.16 (s, 1H), 7.26 (d, J=8.4 Hz, 2H), 7.44 (d, J=8.4 Hz, 2H), 10.91 (s, 1H).

Step 4′. Preparation of tert-butyl (R)-4-hydroxy-4-(4-(3-methyl-2,6-dioxopiperidin-3-yl)phenyl)piperidine-1-carboxylate 6′. To a stirred solution of tert-butyl (R)-4-(4-(3-methyl-2,6-dioxopiperidin-3-yl)phenyl)-3,6-dihydropyridine-1(2H)-carboxylate 5′ (0.5 g, 1.196 mmol) in DCM/ethanol (7:3, 10 mL) was added Mn(dpm)3 (36.5 mg, 0.06 mmol) and the reaction mixture was cooled to 0° C. Phenylsilane (0.295 mL, 2.393 mmol) was added at the same temperature, warmed to room temperature, and the resulting reaction mixture was stirred at room temperature for 12 h under an oxygen bladder. The reaction mixture was quenched with water (100 mL) and extracted with DCM (2×400 mL). The combined organic extracts were dried over sodium sulphate, filtered, and concentrated under reduced pressure to get crude compound. The crude compound was purified by column chromatography using silica gel (230-400 mesh) with 40% ethyl acetate/pet ether to obtain tert-butyl (R)-4-hydroxy-4-(4-(3-methyl-2,6-dioxopiperidin-3-yl)phenyl)piperidine-1-carboxylate 6′ (632 mg, 74.8% yield). LCMS method 3: retention time: 0.836 min and 0.905, [M+H−Boc]⁺=303.2.

Step 5′. Preparation of tert-butyl (R)-4-fluoro-4-(4-(3-methyl-2,6-dioxopiperidin-3-yl)phenyl)piperidine-1-carboxylate 7′. To a stirred solution of tert-butyl (R)-4-hydroxy-4-(4-(3-methyl-2,6-dioxopiperidin-3-yl)phenyl)piperidine-1-carboxylate (0.632 g, 0.895 mmol) in acetonitrile (4 mL) at 0° C. was added deoxo-fluor (1.650 mL, 4.48 mmol) and stirred at room temperature for 4 h. The reaction mixture was quenched by using water (100 mL) and extracted with ethyl acetate (2×300 mL). The combined organic extracts were dried over sodium sulfate, filtered, and concentrated under reduced pressure to obtain the crude compound. The crude compound was purified by column chromatography using silica gel (230-400 mesh) with 60% ethyl acetate/pet ether to obtain tert-butyl (R)-4-fluoro-4-(4-(3-methyl-2,6-dioxopiperidin-3-yl)phenyl)piperidine-1-carboxylate 7′ (220 mg, 29.8% yield). LCMS method 3: retention time: 2.816 min, [(M+H)-100]⁺=305.2.

Step 6′. SFC separation of (R)-3-(4-(4-fluoropiperidin-4-yl)phenyl)-3-methylpiperidine-2,6-dione C-114. To a stirred solution of tert-butyl (R)-4-fluoro-4-(4-(3-methyl-2,6-dioxopiperidin-3-yl)phenyl)piperidine-1-carboxylate 7′ (0.2 g, 0.242 mmol) in DCM (5 mL) at 0° C. was added 4.0 N HCl in 1,4-dioxane (8.83 mg, 0.242 mmol). The resulting reaction mixture was stirred at room temperature for 2 h. The reaction mixture was concentrated under reduced pressure to obtain crude (R)-3-(4-(4-fluoropiperidin-4-yl)phenyl)-3-methylpiperidine-2,6-dione C-114 (155 mg, 116% yield) as brown solid. It was used for the next step without further purification. LCMS method 1: retention time: 2.092 min, [M+H]⁺=305.2.

### Example S74. Preparation of (3R)-3-(4-(3,3-difluoropiperidin-4-yl)phenyl)-3-methylpiperidine-2,6-dione (C-115a)

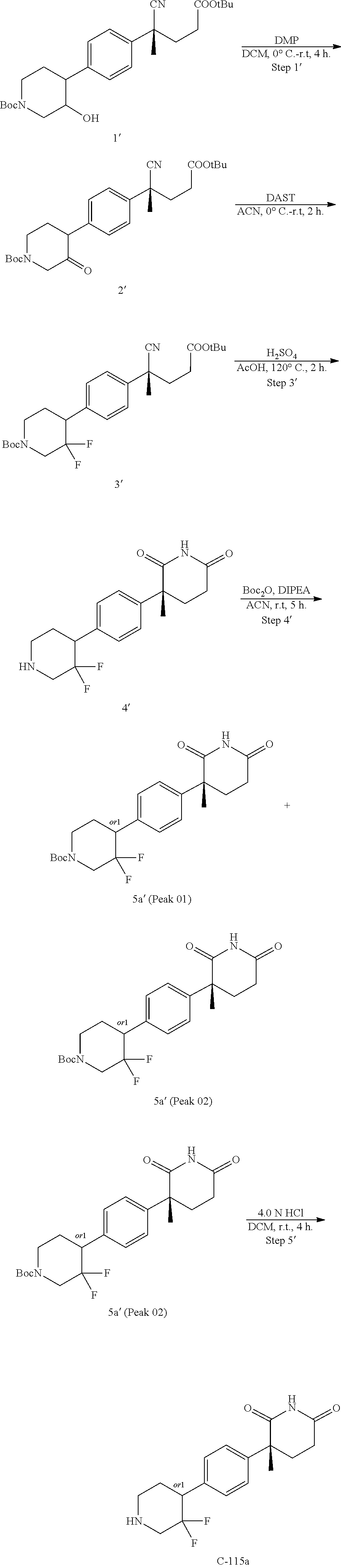

LCMS Method 1. Kinetex XB-C18, 50×4.6 mm, 5.0 Temperature: RT, Flow: 1.0 mL/min, run time: 5.5 min. Mobile Phase Conditions: Mobile Phase-A: 0.1% TFA in H₂O, Mobile Phase-B: 0.1% TFA in ACN, Gradient: Initial 95% Mobile Phase A and 5% Mobile Phase B linear gradient to 95% Mobile Phase B for 2.5 min. MSD positive.

UPLC Method 2. Aquity BEH-C18, 50×2.1 mm, 1.7 Temperature: RT, Flow: mL/min, run time: 2.5 min. Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 98% Mobile Phase B for 1.5 min. MSD positive.

LCMS Method 3. Kinetex Biphenyl, 100×4.6 mm, 2.6 Temperature: RT, Flow: 1.0 mL/min, run time: 15.0 min. Mobile Phase Conditions: Mobile Phase-A: 0.1% TFA in H₂O, Mobile Phase-B: 0.1% TFA in ACN, Gradient: Initial 98% Mobile Phase A and 2% Mobile Phase B linear gradient to 100% Mobile Phase B for 11.0 min. MSD positive.

LCMS Method 4. Kinetex XB-C18, 75×3.0 mm, 2.6 Temperature: RT, Flow: 1.0 mL/min, run time: 5.0 min. Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 98% Mobile Phase A and 2% Mobile Phase B linear gradient to 100% Mobile Phase B for 4 min. MSD positive.

Step 1′. Preparation of tert-butyl 4-(4-(((R)-5-(tert-butoxy)-2-cyano-5-oxopentan-2-yl)phenyl)-3-oxopiperidine-1-carboxylate 2′. To a cooled stirred solution of tert-butyl 4-(4-((R)-5-(tert-butoxy)-2-cyano-5-oxopentan-2-yl)phenyl)-3-hydroxypiperidine-1-carboxylate 1′ (400 mg, 0.768 mmol, 1.0 eq.) and DCM (3 mL) at 0° C. was added DMP (651 mg, 1.535 mmol, 2.0 eq.) and stirred the reaction mass for 4 h at RT. The reaction mixture was quenched with 10% sodium bicarbonate solution (20 mL) slowly at 0° C. and then extracted with EtOAc (3×50 mL). The combined organics were dried over Na₂SO₄ and concentrated under reduced pressure to obtain the crude compound. The crude was purified by column chromatography using silica gel (230-400 mesh) with 20-25% ethyl acetate/pet ether to obtain tert-butyl 4-(4-((R)-5-(tert-butoxy)-2-cyano-5-oxopentan-2-yl)phenyl)-3-oxopiperidine-1-carboxylate 2′ (200 mg, 0.355 mmol, 46.2% yield) as an off-white solid. UPLC Method 2: retention time: 1.550 min, [M−H]⁺=455.2.

Step 2′. Preparation of tert-butyl 4-(4-(((R)-5-(tert-butoxy)-2-cyano-5-oxopentan-2-yl)phenyl)-3,3-difluoropiperidine-1-carboxylate 3′. To a cooled stirred solution of tert-butyl 4-(4-((R)-5-(tert-butoxy)-2-cyano-5-oxopentan-2-yl)phenyl)-3-oxopiperidine-1-carboxylate 2′ (700 mg, 1.272 mmol, 1.0 eq) in DCM (10 mL) at 0° C. was added DAST (0.504 mL, 3.82 mmol, 3.0 eq.). The resulting reaction was slowly warmed to r.t and stirred for 2 h. The reaction mixture was treated with 10% sodium bicarbonate (20 mL) slowly and then extracted with EtOAc (3×50 mL). The combined organics were dried over Na₂SO₄ and concentrated under reduced pressure to obtain the crude compound. The crude was purified by column chromatography using silica gel (230-400 mesh) with 15-20% ethyl acetate/pet ether to obtain tert-butyl 4-(4-((R)-5-(tert-butoxy)-2-cyano-5-oxopentan-2-yl)phenyl)-3,3-difluoropiperidine-1-carboxylate 3′ (310 mg, 0.641 mmol, 50.3% yield) as an off-white solid. LCMS method 4: retention time: 3.058 min, 98.9% purity at 220 nm, [(M+H)-100]⁺=379.2.

Step 3′. Preparation of (3R)-3-(4-(3,3-difluoropiperidin-4-yl)phenyl)-3-methylpiperidine-2,6-dione 4′. To a stirred solution of tert-butyl 4-(4-(((R)-5-(tert-butoxy)-2-cyano-5-oxopentan-2-yl)phenyl)-3,3-difluoropiperidine-1-carboxylate 3′ (100 mg, 0.207 mmol, 1.0 eq) in AcOH (4.0 mL) was added H₂SO₄ (0.022 mL, 0.413 mmol, 2.0 eq.). The resulting reaction mixture was heated to 120° C. and stirred for 2 h. The reaction mixture was concentrated under reduced pressure to obtain crude (3R)-3-(4-(3,3-difluoropiperidin4-yl)phenyl)-3-methylpiperidine-2,6-dione. 2AcOH salt 4′ (210 mg, 0.195 mmol, 94% yield) as a light brown solid. It was used for the next step without further purification. LCMS Method 3: retention time: 9.716 min, [M+H]⁺=323.2.

Step 4′. Preparation and chiral SFC separation of tert-butyl 3,3-difluoro-4-(4-((R)-3-methyl-2,6-dioxopiperidin-3-yl)phenyl)piperidine-1-carboxylate 5a′ (Peak-1) and tert-butyl 3,3-difluoro-4-(4-(((R)-3-methyl-2,6-dioxopiperidin-3-yl)phenyl)piperidine-1-carboxylate 5b′ (Peak-2). To a stirred solution of (3R)-3-(4-(3,3-difluoropiperidin-4-yl)phenyl)-3-methylpiperidine-2,6-dione 4′ (1.0 g, 2.73 mmol, 1.0 eq.) in acetonitrile (10 mL) at RT was added DIPEA (4.77 mL, 27.3 mmol, 10.0 eq.). Then Di-tert-butyl decarbonate (1.268 mL, 5.46 mmol, 2.0 eq.) was added to the reaction mixture, and the resulting reaction mixture was stirred at RT for 3 h. The reaction mixture was concentrated under reduced pressure to obtain the crude product. The crude product was purified by column chromatography using silica gel (230-400 mesh) with 15-20% ethyl acetate/pet ether to obtain the diastereomeric mixture of tert-butyl 3,3-difluoro-4-(4-((R)-3-methyl-2,6-dioxopiperidin-3-yl)phenyl)piperidine-1-carboxylate (700 mg). The racemic compound was purified by chiral SFC to obtain tert-butyl 3,3-difluoro-4-(4-(((R)-3-methyl-2,6-dioxopiperidin-3-yl)phenyl)piperidine-1-carboxylate Peak-1 (300 mg) as an off-white solid and tert-butyl 3,3-difluoro-4-(4-(((R)-3-methyl-2,6-dioxopiperidin-3-yl)phenyl)piperidine-1-carboxylate Peak-2 5b′ (310 mg) as an off-white solid. SFC Method: YMC Cellulose-SC, 250×4.6 mm, 5.0 Flow: 3.0 mL/min. Co-Solvent: IPA. Peak 1: 2.464 min and Peak 2: 3.136 min. tert-butyl 3,3-difluoro-4-(4-((R)-3-methyl-2,6-dioxopiperidin-3-yl)phenyl)piperidine-1-carboxylate Peak-1 5a′: RT 2.464; ee 99.99%. LCMS method 4: retention time: 2.649 min, 96.54% purity at 220 nm, [M+H−100]⁺=323.0. tert-butyl 3,3-difluoro-4-(4-(((R)-3-methyl-2,6-dioxopiperidin-3-yl)phenyl)piperidine-1-carboxylate Peak-2 5b′: RT 3.136; ee 99.99%. LCMS method 4: retention time: 2.649 min, 93.12% purity at 220 nm, [M+H−100]⁺=323.0.

Step 5′. Preparation of (3R)-3-(4-(3,3-difluoropiperidin-4-yl)phenyl)-3-methylpiperidine-2,6-dione. 2HCl C-115a. To a stirred solution of tert-butyl 3,3-difluoro-4-(4-((R)-3-methyl-2,6-dioxopiperidin-3-yl)phenyl)piperidine-1-carboxylate (Peak 01) 5a′ (270 mg, mmol, 1.0 eq.) in DCM (5 mL) at RT was added 4.0 N HCl in 1,4-dioxane (1.542 mL, 6.17 mmol, 10.0 eq.). The resulting reaction mixture was stirred at RT for 4 h. The reaction mixture was concentrated under reduced pressure to obtain crude (3R)-3-(4-(3,3-difluoropiperidin-4-yl)phenyl)-3-methylpiperidine-2,6-dione·2HCl salt C-115a (225 mg, 0.564 mmol, 91% yield) as an off-white solid. It was used for the next step without further purification. LCMS method 1: retention time: 1.601 min, 98.77% purity at 220 nm, [M+H]⁺=323.2. ¹⁹F NMR (376 MHz, DMSO-d6): δ ppm −102.434 (d, 2J_{F}=251.1 Hz), −110.757 (d, 2J_{F}=250.7 Hz).

Table 60 summarizes the intermediate that was synthesized using a procedure similar to Example S74.

TABLE 60
Intermediate Compound Prepared via a Procedure Similar to Example S74.

| Intermediate No. | Structure |
|---|---|
| C-115b (peak 2 of difluoropiperidine separation) | 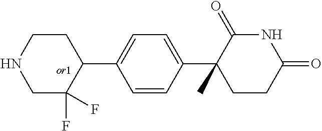 [SMILES: C[C@]1(c2ccc(C3CCNCC3(F)F)cc2)CCC(=O)NC1=O] |

### Example S75. Preparation of 3-(2-oxo-4-(piperidin-4-yl)pyridin-1(2H)-yl)piperidine-2,6-dione (C-116)

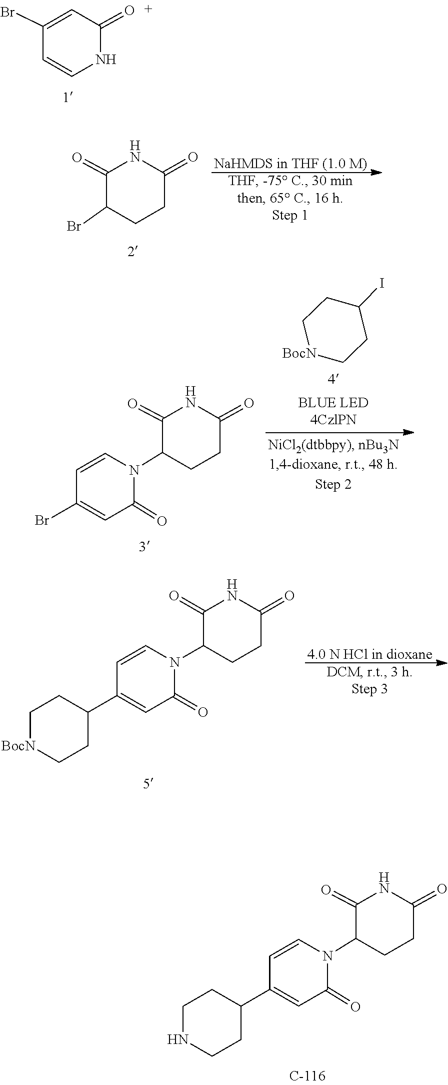 [SMILES: O=c1cc(Br)cc[nH]1 ; O=C1CCC(Br)C(=O)N1 ; CC(C)(C)OC(=O)N1CCC(I)CC1 ; O=C1CCC(n2ccc(Br)cc2=O)C(=O)N1 ; CC(C)(C)OC(=O)N1CCC(c2ccn(C3CCC(=O)NC3=O)c(=O)c2)CC1 ; O=C1CCC(n2ccc(C3CCNCC3)cc2=O)C(=O)N1]

LCMS Method 1. Kinetex XB-C18, 75×3.0 mm, 2.6 Temperature: RT, Flow: 1.0 mL/min, run time: 5.0 min. Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 98% Mobile Phase A and 2% Mobile Phase B linear gradient to 100% Mobile Phase B for 4 min. MSD positive.

UPLC Method 2. Aquity BEH-C18, 50×3.0 mm, 1.7 Temperature: RT, Flow: mL/min, run time: 2.5 min. Mobile Phase Conditions: Mobile Phase-A: 5.0 mm Ammonium formate pH 3.3:CH₃CN (98:02), Mobile Phase-B: CH₃CN: 5.0 mm Ammonium formate pH 3.3 (98:02), Gradient: Initial 80% Mobile Phase A and 20% Mobile Phase B linear gradient to 98% Mobile Phase B for 1.5 min. MSD positive.

Step 1′. Preparation of obtain 3-(4-bromo-2-oxopyridin-1(2H)-yl)piperidine-2,6-dione 3′. To a stirred solution of 4-bromopyridin-2(1H)-one 1′ (5.89 g, 33.9 mmol) in THF (50 mL) was cooled down to −75° C., and NaHMDS (1.0 M THF, 36.5 mL, 36.5 mmol) was added into the reaction mixture. The resulting reaction mixture was stirred at the same temperature for min. Then, 3-bromopiperidine-2,6-dione 2′ (5.0 g, 26.0 mmol) was added to the reaction mixture, and the reaction mixture was stirred at 65° C. for 16 h. The reaction mixture was quenched by adding water (100 mL) and extracted with ethyl acetate (2×400 mL). The combined organic extracts were dried over sodium sulphate, filtered, and concentrated under reduced pressure to get the crude compound. The crude compound was purified by reverse phase column chromatography to obtain 3-(4-bromo-2-oxopyridin-1(2H)-yl)piperidine-2,6-dione 3′ (1.6 g, 20.69% yield) as an off-white solid. LCMS method 1: retention time: 2.000 min, [M+H]⁺=285.0 and [M+H+2]⁺=287.0. ¹H NMR (400 MHz, DMSO-d6): δ ppm 1.98-2.08 (m, 1H), 2.53-2.61 (m, 2H), 2.78-2.87 (m, 1H), 5.41 (brs, 1H), 6.53-6.55 (m, 1H), 6.78 (d, J=2.4 Hz, 1H), 7.65 (d, J=7.2 Hz, 1H), 11.05 (s, 1H). ¹³C NMR (101 MHz, DMSO-d₆): δ ppm 22.9, 31.3, 58.4, 110.1, 121.9, 136.1, 139.7, 160.4, 170.1, 172.9.

Step 2′. Preparation of tert-butyl 4-(1-(2,6-dioxopiperidin-3-yl)-2-oxo-1,2-dihydropyridin-4-yl)piperidine-1-carboxylate 5′. To a stirred solution of 3-(4-bromo-2-oxopyridin-1(2H)-yl)piperidine-2,6-dione 3′ (0.115 g, 0.386 mmol) and tert-butyl 4-iodopiperidine-1-carboxylate 4′ (0.1 g, 0.321 mmol) in 1,4-dioxane (2 mL) was added tributylamine (0.230 mL, 0.964 mmol). The resulting reaction mixture was purged with nitrogen for 10 mins. 4-CzIPN (5.07 mg, 6.43 μmol) and NiCl₂·dtbpy (12.79 mg, 0.032 mmol) were added into the reaction mixture and stirred at room temperature for 48 h under the irradiation of blue led. The reaction mixture was quenched by using ice-cold water (30 mL) and extracted with ethyl acetate (2×80 mL). The combined organic extracts were washed with brine solution (20 mL), dried over sodium sulphate, filtered, and concentrated under reduced pressure to obtain the crude compound. The crude compound was purified by reverse phase column chromatography to obtain tert-butyl 4-(1-(2,6-dioxopiperidin-3-yl)-2-oxo-1,2-dihydropyridin-4-yl)piperidine-1-carboxylate 5′ (140 mg) as an off-white solid. UPLC method 2: retention time: 1.009 min, [(M+H)-56]⁺=334.2.

Step 3′. Preparation of 3-(2-oxo-4-(piperidin-4-yl)pyridin-1(2H)-yl)piperidine-2,6-dione C-116. To a stirred solution of tert-butyl 4-(1-(2,6-dioxopiperidin-3-yl)-2-oxo-1,2-dihydropyridin-4-yl)piperidine-1-carboxylate 5′ (0.140 mg, 0.349 mmol) in DCM (2 mL) at 0° C. was added 4.0 N HCl in 1,4-dioxane (12.71 mg, 0.349 mmol). The resulting reaction mixture was stirred at room temperature for 2 h. The reaction mixture was concentrated under reduced pressure to obtain crude 3-(2-oxo-4-(piperidin-4-yl)pyridin-1(2H)-yl)piperidine-2,6-dione C-116 (110 mg) as a light yellow solid. It was used for the next step without further purification. LCMS method 1: retention time: 0.382 min, [M+H]⁺=290.2.

Table 61 summarizes the Final Compounds Prepared via General Procedure X-10.

TABLE 61
Final Compounds Prepared via General Procedure X-10.

| Compound | TBM | CBM | | |
|---|---|---|---|---|
| No. | protion | portion | Structure | Characterization |
| P-273 | A-29 | C-107 | 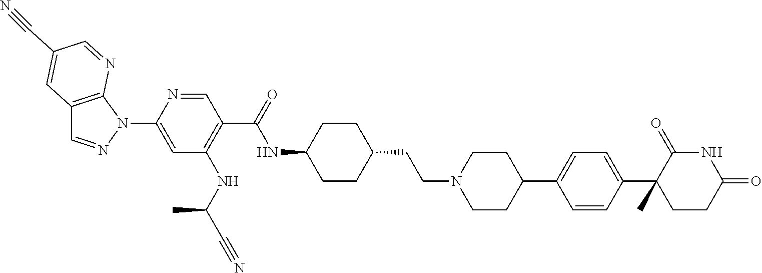 [SMILES: C[C@H](C#N)Nc1cc(-n2ncc3cc(C#N)cnc32)ncc1C(=O)N[C@H]1CC[C@H](CCN2CCC(c3ccc([C@@]4(C)CCC(=O)NC4=O)cc3)CC2)CC1] | 24.6% yield. LCMS method 7, retention time: 1.83 min, 99.4% purity at 220 nm, [M + H]⁺ = 727.4 1H NMR (400 MHz, DMSO-d6): δ ppm 1.05 - 1.13 (m, 2H), 1.37 - 1.41 (m, 3H), 1.44 (s, 3H), 1.61 - 1.62 (m, 2H), 1.68 (d, J = 6.8 Hz, 3H), 1.82 - 2.14 (m, 10H), 2.79 - 2.85 (m, 1H), 2.99 - 3.07 (m, 2H), 3.11 - 3.18 (m, 2H), 3.31 - 3.33 (m, 1H), 3.58 - 3.61 (m, 2H), 3.76 - 3.79 (m, 2H), 4.92 - 4.99 (m, 1H), 7.23 - 7.36 (m, 4H), 7.50 (s, 1H), 8.57 (d, J = 7.6 Hz, 1H), 8.72 (d, J = 8.0 Hz, 2H), 8.77 (d, J = 7.2 Hz, 1H), 9.05 - 9.06 (m, 2H), 10.92 (s, 1H). Two protons are not apparent |
| P-274 | A-29 | C-114 | 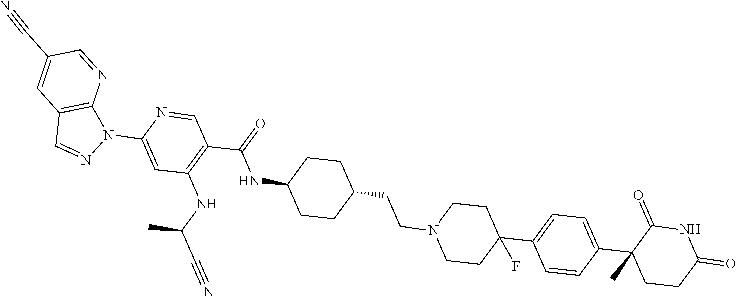 [SMILES: C[C@H](C#N)Nc1cc(-n2ncc3cc(C#N)cnc32)ncc1C(=O)N[C@H]1CC[C@H](CCN2CCC(F)(c3ccc([C@@]4(C)CCC(=O)NC4=O)cc3)CC2)CC1] | 4.6% yield. LCMS method 7, retention time: 1.84 min, 94.8% purity at 220 nm, [M + H]⁺ = 745.2 ¹H NMR (400 MHz, DMSO-d6): δ ppm 1.05 - 1.14 (m, 2H), 1.30 - 1.40 (m, 4H), 1.46 (s, 3H), 1.61 - 1.63 (m, 2H), 1.68 (d, J = 6.8 Hz, 3H), 1.81 - 1.83 (m, 2H), 1.94 (m, 2H), 2.03 - 2.16 (m, 3H), 2.25 - 2.28 (m, 2H), 2.32 - 2.41 (m, 2H), 3.21 - 3.28 (m, 4H), 3.78 - 3.79 (m, 2H), 4.92 - 4.99 (m, 1H), 7.38 - 7.45 (m, 4H), 7.51 (s, 1H), 8.59 (d, J = 6.0 Hz, 1H), 8.71 (d, J = 8.8 Hz, 2H), 8.77 (d, J = 6.8 Hz, 1H), 9.05 (d, J = 2.4 Hz, 2H), 10.95 (s, 1H). One proton is not apparent. ¹⁹F NMR (376 MHz, DMSO-d6): δ ppm −156.59 |
| P-284 | A-29 | C-112a | 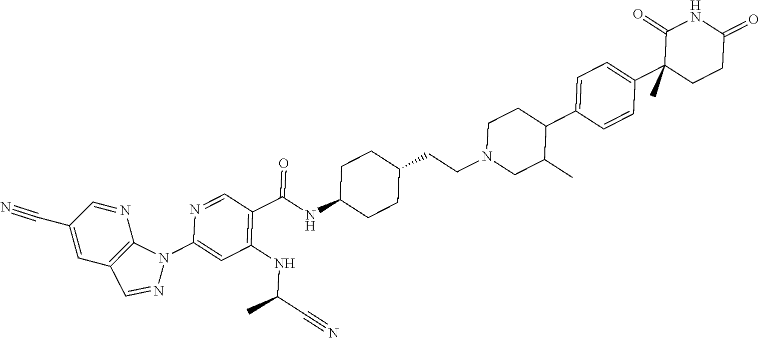 | 27.8% yield. LCMS method 7, retention time: 1.92 min, 97.6% purity at 220 nm, [M + H]⁺ = 765.2 ¹H NMR (400 MHz, DMSO-d6): δ ppm 0.67 (d, J = 6.8 Hz, 3H), 1.11 - 1.27 (m, 2H), 1.45 - 1.53 (m, 6H), 1.62 (d, J = 7.2 Hz, 3H), 1.63- 1.72 (m, 2H), 1.88 - 1.92 (m, 4H), 2.06 - 2.18 (m, 5H), 2.41 - 2.46 (m, 2H), 2.72 - 2.81 (m, 2H), 2.96 - 3.04 (m, 1H), 3.17 - 3.19 (m, 2H), 3.54 - 3.59 (m, 2H), 5.01 - 5.05 (m, 2H), 7.19 - 7.22 (m, 3H), 7.26 - 7.35 (m, 2H), 7.68 (s, 1H), 8.38 (d, J = 5.2 Hz, 1H), 8.42 (d, J = 2.8 Hz, 1H), 8.73 (s, 1H), 9.06 -9.08 (m, 2H), 10.93 (s, 1H) |
| P-272 | A-29 | C-112b | 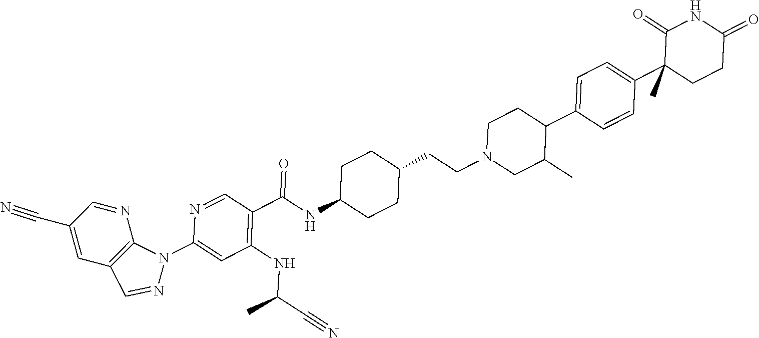 [SMILES: CC1CN(CC[C@H]2CC[C@H](NC(=O)c3cnc(-n4ncc5cc(C#N)cnc54)cc3N[C@H](C)C#N)CC2)CCC1c1ccc([C@@]2(C)CCC(=O)NC2=O)cc1] | 15.2% yield. LCMS method 7, retention time: 1.96 min, 97.3% purity at 220 nm, [M + H]⁺ = 741.4 ¹H NMR (400 MHz, DMSO-d6): δ ppm 0.66 (d, J = 6.4 Hz, 3H), 1.06 - 1.17 (m, 2H), 1.37 - 1.41 (m, 4H), 1.45 (s, 3H), 1.63- 1.69 (m, 5H), 1.82 - 1.85 (m, 2H), 1.93 (m, 4H), 2.03 - 2.08 (m, 3H), 2.34 - 2.46 (m, 1H), 2.67 - 2.78 (m, 1H), 2.92 - 3.08 (m, 1H), 3.14 - 3.15 (m, 2H), 3.55 - 3.57 (m, 2H), 3.76 - 3.85 (m, 1H), 4.94 - 4.98 (m, 1H), 7.19 - 7.21 (m, 2H), 7.26 - 7.34 (m, 2H), 7.49 (s, 1H), 8.57 (d, J = 7.6 Hz, 1H), 8.71 (s, 1H), 8.73 (s, 1H), 8.76 (d, J = 7.2 Hz, 1H), 9.04 -9.06 (m, 2H), 10.93 (s, 1H) One proton is not apparent. |
| P-271 | A-29 | C-106a | 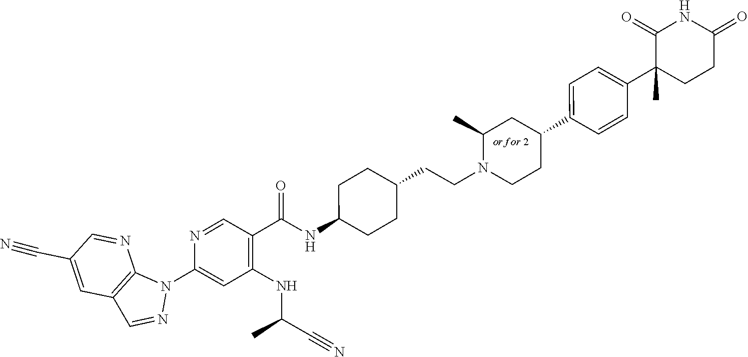 [SMILES: C[C@H](C#N)Nc1cc(-n2ncc3cc(C#N)cnc32)ncc1C(=O)N[C@H]1CC[C@H](CCN2CC[C@@H](c3ccc([C@@]4(C)CCC(=O)NC4=O)cc3)C[C@@H]2C)CC1] | 57.2% yield as an off white solid. LCMS method 13, retention time: 2.30 min, 99.3% purity at 220 nm, [M + H]⁺ = 741.7 ¹H NMR (400 MHz, DMSO-d6): δ 1.09 - 1.12 (m, 2H), 1.35 - 1.42 (m, 5H), 1.44 - 1.48 (m, 4H), 1.58 - 1.60 (m, 2H), 1.68 (d, J = 6.8 Hz, 3H), 1.80 - 2.11 (m, 10H), 2.35 - 2.40 (m, 1H), 3.06 - 3.09 (m, 3H), 3.14 - 3.22 (m, 2H), 3.75 - 3.83 (m, 2H), 4.94 - 4.98 (m, 1H), 7.25 - 7.39 (m, 4H), 7.50 (s, 1H), 8.57 (d, J = 7.60 Hz, 1H), 8.70 (s, 1H), 8.71 (s, 1H), 8.77 (d, J = 7.2 Hz, 1H), 9.04 - 9.06 (m, 2H), 10.92 (s, 1H). One proton is not apparent. |
| P-270 | A-29 | C-106b | 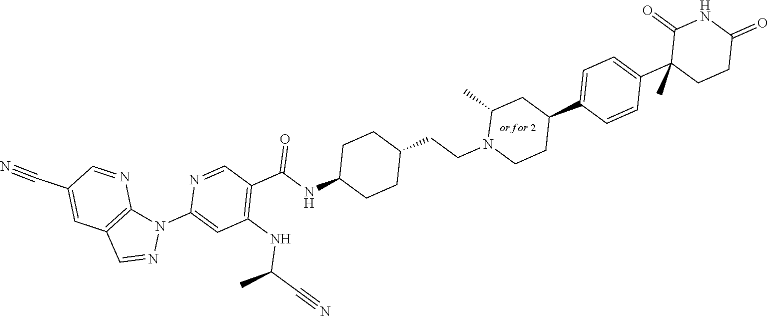 [SMILES: C[C@H](C#N)Nc1cc(-n2ncc3cc(C#N)cnc32)ncc1C(=O)N[C@H]1CC[C@H](CCN2CC[C@H](c3ccc([C@@]4(C)CCC(=O)NC4=O)cc3)C[C@H]2C)CC1] | 44.6% yield as an off white solid. LCMS method 7, retention time: 1.81 min, 96.2% purity at 220 nm, [M + H]⁺ = 741.3 ¹H NMR (400 MHz, DMSO-d6): δ ppm 1.09 - 1.30 (m, 2H), 1.36 - 1.49 (m, 9H), 1.58 - 1.60 (m, 2H), 1.68 (d, J = 6.8 Hz, 3H), 1.80 - 2.00 (m, 6H), 2.01 - 2.18 (m, 3H), 2.33 - 2.40 (m, 1H), 3.06 - 3.09 (m, 2H), 3.12 - 3.25 (m, 1H), 3.35 - 3.45 (m, 1H), 4.92 - 5.04 (m, 1H), 7.24 - 7.39 (m, 4H), 7.50 (s, 1H), 8.56 - 8.58 (m, 1H), 8.71 - 8.73 (m, 2H), 8.77 - 8.78 (m, 1H), 9.05 - 9.06 (m, 2H), 10.91 (s, 1H). Five protons are not apparent. |
| P-425 | A-29 | C-116 | 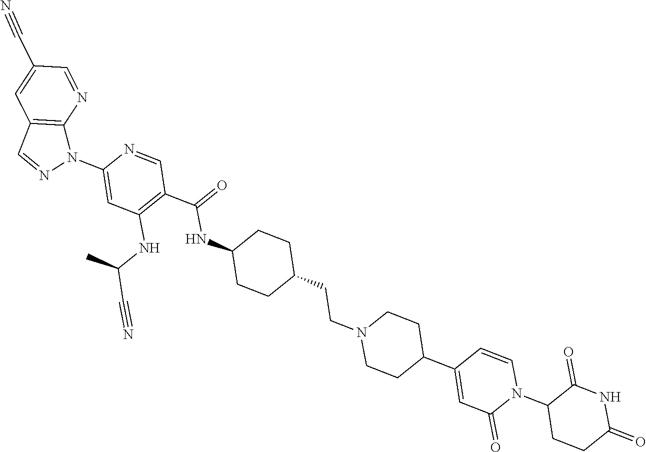 [SMILES: C[C@H](C#N)Nc1cc(-n2ncc3cc(C#N)cnc32)ncc1C(=O)N[C@H]1CC[C@H](CCN2CCC(c3ccn(C4CCC(=O)NC4=O)c(=O)c3)CC2)CC1] | 10.6% yield as a white solid. LCMS method 13, retention time: 1.83 min, 98.5% purity at 220 nm, [M + H]⁺ = 730.3 ¹H NMR (400 MHz, DMSO d6): δ ppm 1.02 - 1.11 (m, 2H), 1.32 - 1.41 (m, 3H), 1.58 - 1.62 (m, 2H), 1.67 (d, J = 6.8 Hz, 3H), 1.71 - 1.81 (m, 4H), 1.90 - 1.98 (m, 3H), 2.04 - 2.07 (m, 2H), 2.55 - 2.60 (m, 1H), 2.78 - 2.85 (m, 1H), 2.97 - 3.03 (m, 2H), 3.11 - 3.17 (m, 2H), 3.29 - 3.32 (m, 1H), 3.58 - 3.61 (m, 2H), 4.91 - 4.99 (m, 1H), 6.23 - 6.25 (m, 2H), 7.49 (s, 1H), 7.61 (d, J = 7.2 Hz, 1H), 8.56 (d, J = 7.6 Hz, 1H), 8.70 (s, 1H), 8.72 (s, 1H), 8.76 (d, J = 7.6 Hz, 1H), 9.04 - 9.05 (m, 2H), 11.01 (s, 1H). Three protons are not apparent. |
| P-430 | A-29 | C-46 | 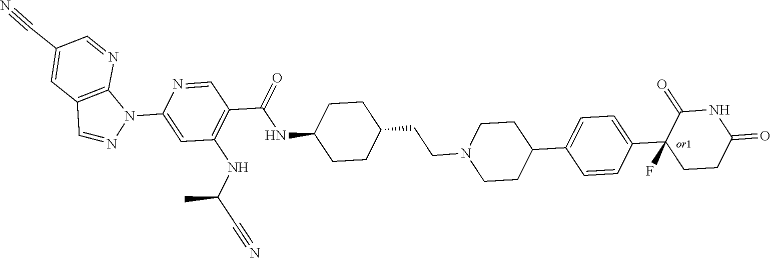 [SMILES: C[C@H](C#N)Nc1cc(-n2ncc3cc(C#N)cnc32)ncc1C(=O)N[C@H]1CC[C@H](CCN2CCC(c3ccc([C@@]4(F)CCC(=O)NC4=O)cc3)CC2)CC1] | 12.0% yield. LCMS method 7, retention time: 1.74 min, 96.0% purity at 220 nm, [M + H]⁺ = 731.2 ¹H NMR (400 MHz, DMSO d6): δ ppm 1.02 - 1.11 (m, 2H), 1.30 - 1.41 (m, 3H), 1.61 - 1.64 (m, 2H), 1.68 (d, J = 6.8 Hz, 3H), 1.80 - 1.94 (m, 6H), 2.02 - 2.07 (m, 2H), 2.28 - 2.31 (m, 1H), 2.65 - 2.76 (m, 2H), 2.86 - 2.91 (m, 1H), 3.01 - 3.09 (m, 2H), 3.12 - 3.18 (m, 2H), 3.31 - 3.34 (m, 1H), 3.61 - 3.63 (m, 2H), 4.91 - 4.99 (m, 1H), 7.36 (d, J = 8.4 Hz, 2H), 7.44 (d, J = 8.4 Hz, 2H), 7.50 (s, 1H), 8.57 (d, J = 7.6 Hz, 1H), 8.71 (s, 1H), 8.73 (s, 1H), 8.77 (d, J = 7.6 Hz, 1H), 9.05 - 9.06 (m, 2H), 11.39 (s, 1H). One proton is not apparent. |

Table 62 summarizes the Final Compounds prepared via General Procedure X-11.

TABLE 62
Final Compounds Prepared via General Procedure X-11

| Com- | TBM | CBM | | |
|---|---|---|---|---|
| pound | por- | por- | | |
| No. | tion | tion | Structure | Characterization |
| P-281 | A-57 | C-90 | 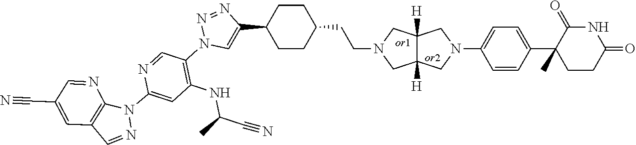 [SMILES: C[C@H](C#N)Nc1cc(-n2ncc3cc(C#N)cnc32)ncc1-n1cc([C@H]2CC[C@H](CCN3C[C@@H]4CN(c5ccc([C@@]6(C)CCC(=O)NC6=O)cc5)C[C@@H]4C3)CC2)nn1] | 25.7% yield. LCMS method 12, retention time: 1.63 min, 99.4% purity at 220 nm, [M + H]⁺ = 778.1 ¹H NMR (500 MHz, DMSO-d6) δ 10.19 - 9.84 (m, 1H), 8.86 - 8.78 (m, 2H), 8.48 (s, 1H), 8.17 (s, 1H), 8.13 (s, 1H), 7.44 (s, 1H), 6.93 (br s, 4H), 6.54 - 6.29 (m, 2H), 4.83 - 4.68 (m, 1H), 4.38 - 4.19 (m, 1H), 4.13 - 3.97 (m, 1H), 3.45 (br t, J = 6.7 Hz, 3H), 3.14 - 2.96 (m, 1H), 2.45 (br t, J = 6.7 Hz, 4H), 2.18 - 2.04 (m, 2H), 1.96 - 1.88 (m, 2H), 1.70 - 1.60 (m, 2H), 1.48 - 1.40 (m, 3H), 1.38 (d, J = |
| | | | | 6.9 Hz, 4H), 1.33 - 1.14 (m, 4H), 1.03 - 0.87 |
| | | | | (m, 2H) |
| P-282 | A-57 | C-106b | 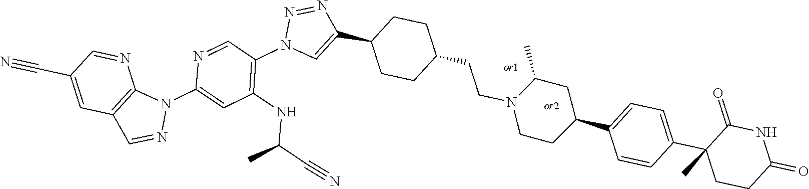 [SMILES: C[C@H](C#N)Nc1cc(-n2ncc3cc(C#N)cnc32)ncc1-n1cc([C@H]2CC[C@H](CCN3CC[C@H](c4ccc([C@@]5(C)CCC(=O)NC5=O)cc4)C[C@H]3C)CC2)nn1] | 31.6% yield as an off white solid. LCMS method 7, retention time: 1.97 min, 97.9% purity at 220 nm, [M + H]⁺ = 765.4 ¹H NMR (400 MHz, DMSO-d6): δ ppm 1.12 - 1.28 (m, 2H), 1.35 - 1.55 (m, 9H), 1.60 - 1.65 (m, 5H), 1.75 - 2.20 (m, 10H), 2.70 - 2.80 (m, 1H), 3.04 - 3.20 (m, 2H), 3.80 - 3.90 (m, 1H), 5.00 - 5.08 (m, 1H), 7.18 - 7.20 (m, 1H), 7.25 - 7.39 (m, 4H), 7.67 (s, 1H), 8.37 (s, 1H), 8.41(s, 1H), 8.72 (s, 1H), 9.05 - 9.07 (m, 2H), 10.91 (s, 1H). Five protons are not apparent. |
| P-283 | A-57 | C-106a | 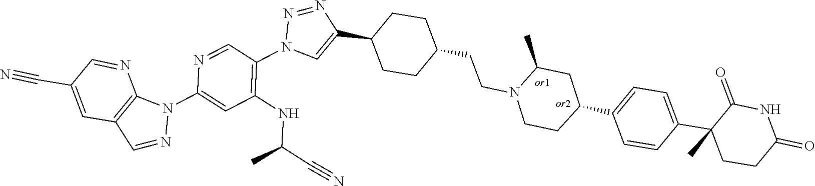 [SMILES: C[C@H](C#N)Nc1cc(-n2ncc3cc(C#N)cnc32)ncc1-n1cc([C@H]2CC[C@H](CCN3CC[C@@H](c4ccc([C@@]5(C)CCC(=O)NC5=O)cc4)C[C@@H]3C)CC2)nn1] | 25.4% yield. LCMS method 13, retention time: 2.36 min, 94.5% purity at 220 nm, [M + H]⁺ = 765.7 ¹H NMR (400 MHz, DMSO-d6): δ ppm 1.17 - 1.24 (m, 2H), 1.37 (d, J = 6.8 Hz, 3H), 1.43 (s, 3H), 1.45 - 1.52 (m, 2H), 1.61 (d, J = 7.2 Hz, 3H), 1.63 - 1.67 (m, 2H), 1.80 - 1.94 (m, 4H), 1.99 - 2.12 (m, 3H), 2.14 - 2.18 (m, 2H), 2.38 - 2.48 (m, 2H), 2.70 - 2.77 (m, 1H), 3.05 - 3.11 (m, 3H), 3.16 - 3.25 (m, 1H) 3.39 - 3.45 (m, 2H), 3.85 - 3.90 (m, 1H), 5.00 - 5.04 (m, |
| | | | | 1H), 7.18 - 7.20 (m, 1H), 7.25 - 7.38 (m, 4H), |
| | | | | 7.67 (s, 1H), 8.37 (s, 1H), 8.41 (s, 1H), 8.72 |
| | | | | (s, 1H), 9.05 - 9.07 (m, 2H), 10.91 (s, 1H). |
| | | | | Three protons are not apparent. |
| P-285 | A-57 | C-112b | 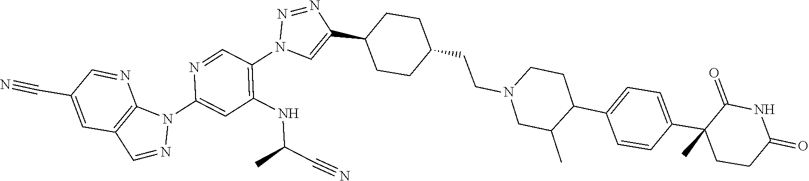 [SMILES: CC1CN(CC[C@H]2CC[C@H](c3cn(-c4cnc(-n5ncc6cc(C#N)cnc65)cc4N[C@H](C)C#N)nn3)CC2)CCC1c1ccc([C@@]2(C)CCC(=O)NC2=O)cc1] | 8.0% yield. LCMS method 13, retention time: 2.49 min, 95.9% purity at 220 nm, [M + H]⁺ = 764.7 ¹H NMR (400 MHz, DMSO-d6): δ ppm 0.67 (d, J = 6.4 Hz, 3H), 1.18 - 1.24 (m, 2H), 1.45 (s, 3H), 1.42 - 1.53 (m, 3H), 1.61 (d, J = 7.8 Hz, 3H), 1.66- 1.68 (m, 2H), 1.88 - 1.91 (m, 4H), 2.03 - 2.18 (m, 6H), 2.41 - 2.46 (m, 2H), 2.74 - 2.78 (m, 2H), 3.02 - 3.08 (m, 1H), 3.17 - 3.19 (m, 2H), 3.54 - 3.58 (m, 2H), 5.01 - 5.05 (m, 1H), 7.19 - 7.22 (m, 3H), 7.28 - 7.29 (m, 2H), 7.68 (s, 1H), 8.37 (s, 1H), 8.42 (s, |
| | | | | 1H), 8.73 (s, 1H), 9.06 - 9.08 (m, 2H), 10.93 |
| | | | | (s, 1H) |
| P-310 | A-57 | C-113a | 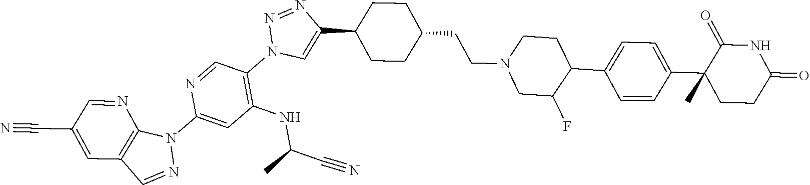 [SMILES: C[C@H](C#N)Nc1cc(-n2ncc3cc(C#N)cnc32)ncc1-n1cc([C@H]2CC[C@H](CCN3CCC(c4ccc([C@@]5(C)CCC(=O)NC5=O)cc4)C(F)C3)CC2)nn1] | 19.0% yield. LCMS method 7, retention time: 1.59 min, 93.9% purity at 220 nm, [M + H]⁺ = 769.2 ¹H NMR (400 MHz, DMSO-d6): δ ppm 1.11 - 1.21 (m, 3H), 1.41 - 1.58 (m, 6H), 1.61 (d, J = 6.8 Hz, 3H), 1.65 - 1.69 (m, 2H), 1.88 - 1.91 (m, 3H), 2.04 - 2.18 (m, 6H), 2.35 - 2.40 (m, 1H), 2.72 - 2.79 (m, 1H), 3.01 - 3.09 (m, 2H), 3.25 - 3.29 (m, 2H), 3.58 - 3.62 (m, 1H), 3.91 - 3.93 (m, 1H), 4.87 (brs, 1H), 4.97 - 5.05 (m, 1H), 7.19 (d, J = 8.0 Hz, 1H), 7.31 - 7.33 (m, 4H), 7.68 (s, 1H), 8.37 (s, 1H), 8.41 |
| | | | | (s, 1H), 8.72 (s, 1H), 9.05 - 9.07 (m, 2H), |
| | | | | 10.93 (s, 1H). |
| | | | | ¹⁹F NMR (376 MHz, DMSO-d6): δ ppm |
| | | | | −184.2 |
| P-311 | A-57 | C-113b | 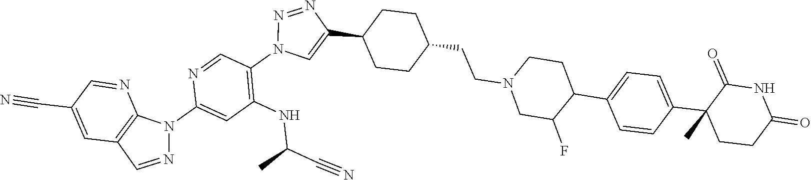 [SMILES: C[C@H](C#N)Nc1cc(-n2ncc3cc(C#N)cnc32)ncc1-n1cc([C@H]2CC[C@H](CCN3CCC(c4ccc([C@@]5(C)CCC(=O)NC5=O)cc4)C(F)C3)CC2)nn1] | 27.4% yield. LCMS method 7, retention time: 1.85 min, 94.0% purity at 220 nm, [M + H]⁺ = 769.2 ¹H NMR (400 MHz, DMSO-d6): δ ppm 1.12 - 1.28 (m, 2H), 1.35 - 1.59 (m, 6H), 1.62 (d, J = 7.2 Hz, 3H), 1.62 - 1.69 (m, 2H), 1.88 -1.91 (m, 3H), 2.02 - 2.18 (m, 5H), 2.34 - 2.38 (m, 1H), 2.74 - 2.82 (m, 1H), 3.02 - 3.12 (m, 2H), 3.16 - 3.30 (m, 3H), 3.58 - 3.61 (m, 1H), 3.91 - 3.96 (m, 1H), 4.82 - 5.08 (m, 2H), 7.19 (d, J = 8.0 Hz, 1H), 7.29 - 7.32 (m, 4H), 7.68 (s, |
| | | | | 1H), 8.37 (s, 1H), 8.42 (s, 1H), 8.73 (s, 1H), |
| | | | | 9.06 - 9.08 (m, 2H), 10.93 (s, 1H). |
| | | | | One proton is not apparent. |
| | | | | ¹⁹F NMR (376 MHz, DMSO-d6): δ ppm |
| | | | | −184.19 |
| P-314 | A-57 | C-115a | 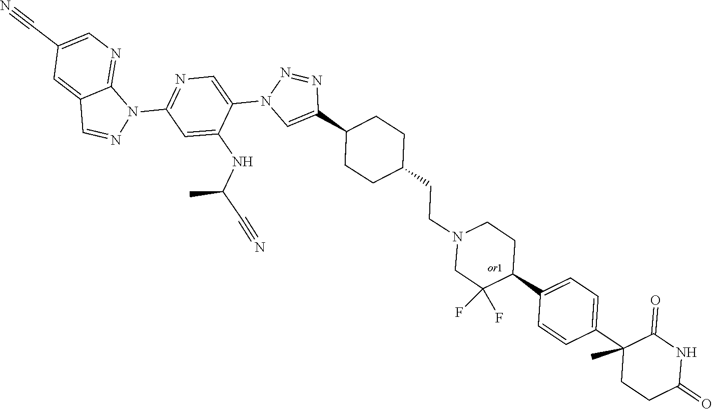 [SMILES: C[C@H](C#N)Nc1cc(-n2ncc3cc(C#N)cnc32)ncc1-n1cc([C@H]2CC[C@H](CCN3CC[C@@H](c4ccc([C@@]5(C)CCC(=O)NC5=O)cc4)C(F)(F)C3)CC2)nn1] | 30.3% yield. LCMS method 13, retention time: 2.37 min, 94.3% purity at 220 nm, [M + H]⁺ = 786.7 ¹H NMR (400 MHz, DMSO-d6): δ ppm 1.10 - 1.25 (m, 2H), 1.40 - 1.58 (m, 6H), 1.62 (d, J = 6.8 Hz, 3H), 1.63 - 1.70 (m, 2H), 1.85 - 1.88 (m, 2H), 2.02 - 2.20 (m, 6H), 2.30 - 2.42 (m, 1H), 2.71 - 2.82 (m, 1H), 3.10 - 3.33 (m, 3H), 4.00 - 4.10 (m, 1H), 4.97 - 5.05 (m, 1H), 7.19 - 7.21 (m, 1H), 7.33 (bs, 4H), 7.68 (s, 1H), 8.37 (s, 1H), 8.42 (s, 1H), 8.73 (s, 1H), 9.06 - 9.08 (m, 2H), 10.95 (s, 1H). Four protons are not apparent. ¹⁹F NMR (376 MHz, DMSO-d6): δ ppm - 101.692 (d, 2J_{F} = 256 Hz), −109.004 (d, 2J_{F} = 254.5 Hz) |
| P-316 | A-113 | C-46i | 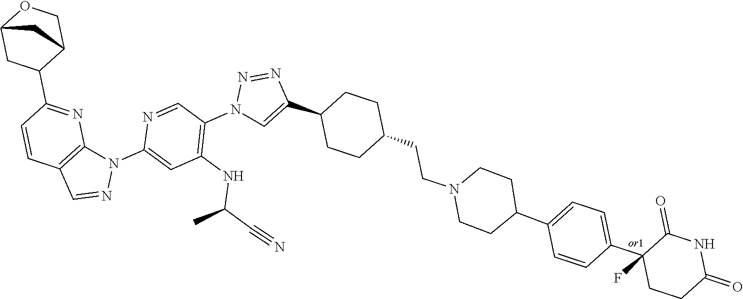 [SMILES: C[C@H](C#N)Nc1cc(-n2ncc3ccc(C4C[C@H]5C[C@@H]4CO5)nc32)ncc1-n1cc([C@H]2CC[C@H](CCN3CCC(c4ccc([C@@]5(F)CCC(=O)NC5=O)cc4)CC3)CC2)nn1] | 3.3% yield. LCMS method 7, retention time: 1.60 min, 94.7% purity at 220 nm, [M + H]⁺ = 827.2 ¹H NMR (400 MHz, DMSO-d6): δ ppm 1.16 - 1.24 (m, 2H), 1.39 - 1.48 (m, 3H), 1.60- 1.71 (m, 5H), 1.75 - 2.04 (m, 6H), 2.08 - 2.13 (m, 4H), 2.32 - 2.37 (m, 1H), 2.67 - 2.76 (m, 2H), 2.89 - 2.95 (m, 1H), 3.02 - 3.07 (m, 2H), 3.10 - 3.18 (m, 2H), 3.61 - 3.64 (m, 2H), 3.75 - 3.79 (m, 2H) 4.73 (s, 1H), 5.00 - 5.05 (m, 1H), 7.09 - 7.25 (m, 2H), 7.34 - 7.37 (m, 2H), 7.43 - 7.47 (m, 2H), 7.93 (s, 1H), 8.30 (s, 1H), 9.15 (s, 1H), 11.39 (s, 1H). Seven protons are not apparent. |
| P-317 | A-66 | C-46 | 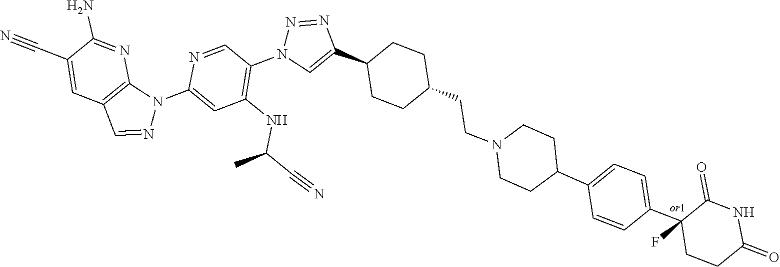 [SMILES: C[C@H](C#N)Nc1cc(-n2ncc3cc(C#N)c(N)nc32)ncc1-n1cc([C@H]2CC[C@H](CCN3CCC(c4ccc([C@@]5(F)CCC(=O)NC5=O)cc4)CC3)CC2)nn1] | 22.1% yield. LCMS method 12, retention time: 1.45 min, 100% purity at 220 nm, [M + H]⁺ = 770.2 ¹H NMR (500 MHz, DMSO-d6) δ 8.57 (s, 1H), 8.36 - 8.30 (m, 2H), 8.24 (s, 1H), 7.98 (s, 1H), 7.35 (br s, 6H), 7.11 - 7.05 (m, 1H), 5.06 - 4.92 (m, 1H), 2.76 - 2.56 (m, 3H), 2.41 - 2.24 (m, 3H), 2.21 - 2.05 (m, 2H), 1.99 - 1.88 (m, 6H), 1.88 - 1.84 (m, 1H), 1.84 - 1.79 (m, 1H), 1.78 - 1.71 (m, 2H), 1.71 - 1.58 (m, 6H), 1.57 - 1.37 (m, 3H), 1.17 - 0.99 (m, 2H), 0.86 (br d, J = 12.2 Hz, 1H) ¹⁹F NMR (471 MHz, DMSO-d6) δ −146.38 (s, 1F) |
| P-318 | A-66 | C-46i | 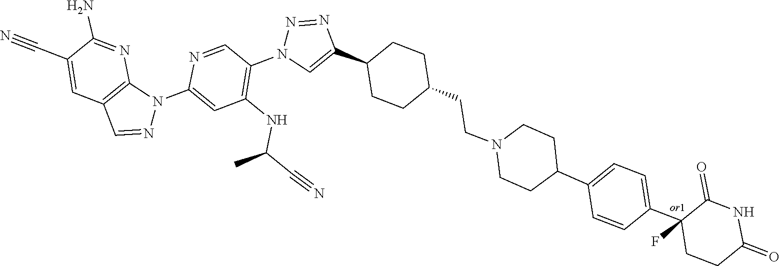 [SMILES: C[C@H](C#N)Nc1cc(-n2ncc3cc(C#N)c(N)nc32)ncc1-n1cc([C@H]2CC[C@H](CCN3CCC(c4ccc([C@@]5(F)CCC(=O)NC5=O)cc4)CC3)CC2)nn1] | 42.1% yield. LCMS method 12, retention time: 1.34 min, 98.2% purity at 220 nm, [M + H]⁺ = 770.2 ¹H NMR (500 MHz, DMSO-d6) δ 11.39 (s, 1H), 8.57 (s, 1H), 8.33 (br d, J = 4.0 Hz, 2H), 8.24 (s, 1H), 7.99 (s, 1H), 7.48 - 7.38 (m, 2H), 7.35 (br d, J = 4.6 Hz, 2H), 7.13 - 7.04 (m, 1H), 5.05 - 4.93 (m, 1H), 3.62 (br d, J = 11.1 Hz, 1H), 3.43 (br d, J = 5.0 Hz, 1H), 3.17 (s, 1H), 3.05 (br d, J = 10.1 Hz, 2H), 3.01 - 2.83 (m, 2H), 2.75 (br s, 1H), 2.73 - 2.61 (m, 3H), 2.34 (br s, 1H), 2.15 (br d, J = 12.9 Hz, 1H), 2.05 (br d, J = 15.5 Hz, 1H), 2.01 - 1.92 (m, 1H), 1.87 (br s, 3H), 1.62 (br dd, J = 6.7, 4.0 Hz, 5H), 1.57 - 1.36 (m, 2H), 1.17 (br d, J = 14.1 Hz, 1H), 1.11 - 0.95 (m, 1H) ¹⁹F NMR (471 MHz, DMSO-d6) δ −147.25 (s, |
| | | | | 1F) |
| P-319 | A-112 | C-46i | 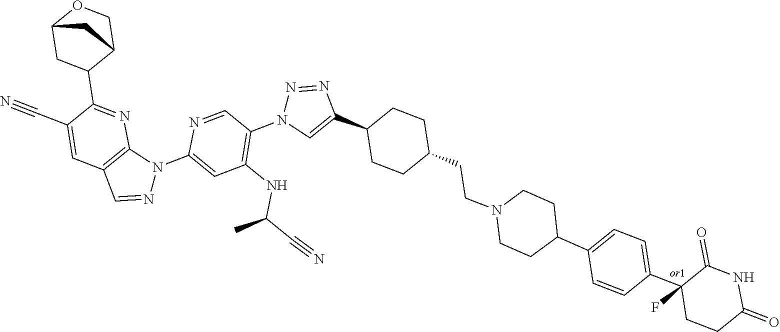 [SMILES: C[C@H](C#N)Nc1cc(-n2ncc3cc(C#N)c(C4C[C@H]5C[C@@H]4CO5)nc32)ncc1-n1cc([C@H]2CC[C@H](CCN3CCC(c4ccc([C@@]5(F)CCC(=O)NC5=O)cc4)CC3)CC2)nn1] | 19.3% yield. LCMS method 7, retention time: 1.77 min, 97.1% purity at 220 nm, [M + H]⁺ = 852.2 ¹H NMR (400 MHz, DMSO-d6): δ ppm 1.17 - 1.19 (m, 2H), 1.49 - 1.68 (m, 8H), 1.89 - 1.97 (m, 5H), 2.04 - 2.14 (m, 3H), 2.17 - 2.22 (m, 2H), 2.25 - 2.38 (m, 1H), 2.61 - 2.73 (m, 3H), 2.76 - 2.98 (m, 1H), 3.04 - 3.08 (m, 2H), 3.12 - 3.18 (m, 2H), 3.61 - 3.67 (m, 3H), 3.85- 3.87 (m, 1H), 3.95 - 3.97 (m, 2H), 4.74 (s, 1H), 4.93 - 4.98 (m, 1H), 5.31 (s, 1H), 7.15 (s, 1H), 7.36 (d, J = 8.0 Hz, 2H), 7.43 - 7.49 (m, 3H), 7.80 (brs, 1H), 8.34 (s, 2H), 8.67 (s, 1H), 11.39 (s, 1H) One proton is not apparent. ¹⁹F NMR (376 MHz, DMSO-d6): δ ppm −147.2 |
| P-320 | A-112 | C-46 | 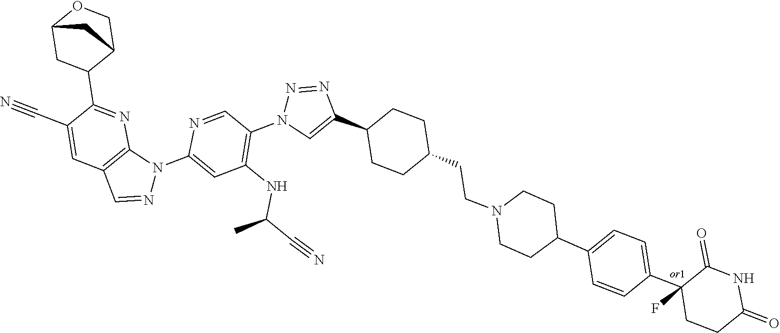 [SMILES: C[C@H](C#N)Nc1cc(-n2ncc3cc(C#N)c(C4C[C@H]5C[C@@H]4CO5)nc32)ncc1-n1cc([C@H]2CC[C@H](CCN3CCC(c4ccc([C@@]5(F)CCC(=O)NC5=O)cc4)CC3)CC2)nn1] | 3.6% yield. LCMS method 12, retention time: 2.13 min, 95.8% purity at 220 nm, [M + H]⁺ = 852.3 ¹H NMR (400 MHz, DMSO-d6): δ ppm 1.12 - 1.21 (m, 2H), 1.39 (brs, 1H), 1.45 - 1.54 (m, 2H), 1.61 (d, J = 6.8 Hz, 3H), 1.65 - 1.67 (m, 2H), 1.81 - 1.90 (m, 5H), 1.96 - 2.00 (m, 1H), 2.05 - 2.08 (m, 2H), 2.15 - 2.18 (m, 2H), 2.35 - 2.37 (m, 1H), 2.71 - 2.76 (m, 2H), 2.85 - 2.92 (m, 1H), 3.02 - 3.10 (m, 2H), 3.15 - 3.21 (m, 2H), 3.61 - 3.68 (m, 3H), 3.85 - 3.88 (m, 1H), 3.95 - 3.99 (m, 2H), 4.74 (m, 1H), 4.95 (brs, 1H), 5.31 (s, 1H), 7.12 - 7.15 (m, 1H), 7.36 (d, J = 8.0 Hz, 2H), 7.44 (d, J = 8.4 Hz, 2H), 7.77 (brs, 1H), 8.32 - 8.36 (m, 3H), 8.69 (s, 1H), 11.39 (s, 1H). Two protons are not apparent. ¹⁹F NMR (376 MHz, DMSO-d6): δ ppm −147.24 |
| P-325 | A-57 | C-83b | 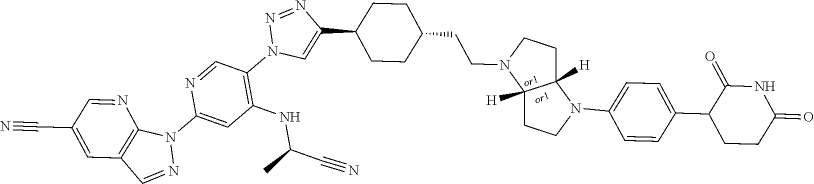 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc(C4CCC(=O)NC4=O)cc3)CC2)nn1] | 29.0% yield. LCMS method 12, retention time: 1.42 min, 100% purity at 220 nm, [M + H]⁺ = 764.2 1H NMR (500 MHz, DMSO-d6) δ 10.77 (s, 1H), 9.09 - 9.03 (m, 2H), 8.72 (s, 1H), 8.41 (s, 1H), 8.39 - 8.35 (m, 1H), 7.68 (s, 1H), 7.26 - 7.17 (m, 1H), 7.14 (s, 1H), 7.11 - 7.02 (m, 2H), 6.64 (br d, J = 8.2 Hz, 2H), 5.02 (s, 1H), 4.48 (br d, J = 6.8 Hz, 1H), 4.27 (br s, 1H), 3.76 - 3.61 (m, 2H), 3.24 (br s, 1H), 2.81 - 2.72 (m, 1H), 2.70 - 2.58 (m, 2H), 2.46 (br s, 1H), 2.40 - 2.29 (m, 2H), 2.20 - 2.10 (m, 3H), |
| | | | | 2.01 (br dd, J = 13.4, 4.9 Hz, 1H), 1.95 - 1.84 |
| | | | | (m, 2H), 1.71 - 1.60 (m, 6H), 1.59 - 1.45 (m, |
| | | | | 2H), 1.45 - 1.36 (m, 1H), 1.27 - 1.13 (m, 2H) |
| P-326 | A-57 | C-105 | 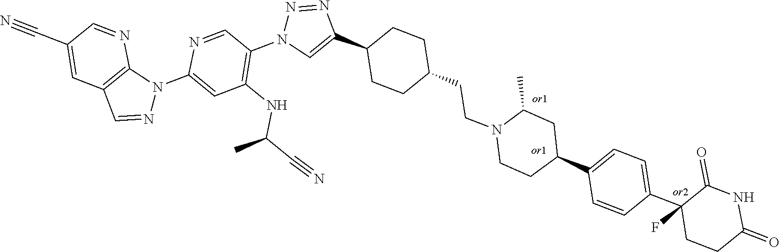 [SMILES: C[C@H](C#N)Nc1cc(-n2ncc3cc(C#N)cnc32)ncc1-n1cc([C@H]2CC[C@H](CCN3CC[C@H](c4ccc([C@@]5(F)CCC(=O)NC5=O)cc4)C[C@H]3C)CC2)nn1] | 53.6% yield. LCMS method 12, retention time: 1.45 min, 83.3% purity at 220 nm, [M + H]⁺ = 769.4 ¹H NMR (500 MHz, DMSO-d6) δ 11.39 (s, 1H), 9.09 - 9.02 (m, 2H), 8.71 (s, 1H), 8.41 (s, 1H), 8.36 (s, 1H), 7.67 (s, 1H), 7.49 - 7.39 (m, 3H), 7.39 - 7.32 (m, 2H), 7.24 - 7.16 (m, 1H), 5.05 - 4.98 (m, 1H), 3.88 - 3.82 (m, 1H), 3.10 (br d, J = 8.7 Hz, 2H), 2.80 - 2.63 (m, 3H), 2.37 - 2.24 (m, 1H), 2.16 (br d, J = 12.6 Hz, 2H), 2.12 - 2.04 (m, 1H), 2.02 - 1.81 (m, 5H), 1.61 (br d, J = 6.9 Hz, 5H), 1.38 (br d, J = 7.0 Hz, 3H), 1.26 - 1.14 (m, 2H) ¹⁹F NMR (471 MHz, DMSO-d6) δ −147.07 (s, 1F) |
| P-327 | A-57 | C-83a | 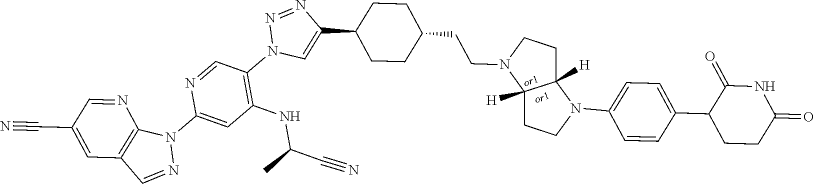 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc(C4CCC(=O)NC4=O)cc3)CC2)nn1] | 19.8% yield. LCMS method 12, retention time: 1.66 min, 98.8% purity at 220 nm, [M + H]⁺ = 764.1 ¹H NMR (500 MHz, DMSO-d6) δ 10.79 - 10.68 (m, 1H), 9.08 - 9.03 (m, 2H), 8.73 - 8.70 (m, 1H), 8.41 (s, 1H), 8.35 (s, 1H), 7.68 (s, 1H), 7.20 (br d, J = 7.9 Hz, 1H), 6.99 (d, J = 8.5 Hz, 2H), 6.51 (br d, J = 8.5 Hz, 2H), 5.07 - 4.96 (m, 1H), 4.16 (br s, 1H), 3.68 (br dd, J = 10.7, 4.9 Hz, 1H), 3.40 (br d, J = 7.4 Hz, 1H), 2.99 (br s, 1H), 2.90 - 2.68 (m, 2H), 2.67 - 2.57 (m, 1H), 2.47 (s, 1H), 2.33 - 2.05 |
| | | | | (m, 6H), 2.05 - 1.95 (m, 1H), 1.85 (br s, 2H), |
| | | | | 1.62 (d, J = 7.0 Hz, 3H), 1.53 - 1.34 (m, 6H), |
| | | | | 1.20 - 1.08 (m, 2H) |
| P-331 | A-57 | C-111b | 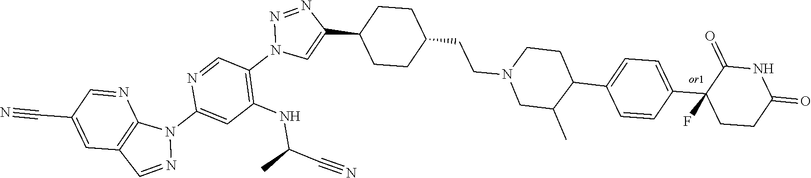 [SMILES: CC1CN(CC[C@H]2CC[C@H](c3cn(-c4cnc(-n5ncc6cc(C#N)cnc65)cc4N[C@H](C)C#N)nn3)CC2)CCC1c1ccc([C@@]2(F)CCC(=O)NC2=O)cc1] | 25.2% yield as an off white solid. LCMS method 7, retention time: 1.85 min, 93.1% purity at 220 nm, [M + H]⁺ = 769.2 ¹H NMR (400 MHz, DMSO-d6): δ ppm 0.68 (d, J = 6.8 Hz, 3H), 1.14 - 1.23 (m, 3H), 1.42 - 1.53 (m, 2H), 1.61 (d, J = 7.2 Hz, 3H), 1.64 - 1.66 (m, 2H), 1.88 - 1.94 (m, 4H), 2.07 - 2.18 (m, 2H), 2.71 - 2.80 (m, 2H), 2.99 - 3.04 (m, 1H), 3.17 - 3.19 (m, 4H), 3.59 - 3.64 (m, 2H), 5.00 - 5.04 (m, 1H), 7.19 (d, J = 7.2 Hz, 1H), 7.29 (d, J = 8.0 Hz, 2H), 7.44 (d, J = 8.4 Hz, |
| | | | | 2H), 7.68 (s, 1H), 8.37 (s, 1H), 8.41 (s, 1H), |
| | | | | 8.72 (s, 1H), 9.05 - 9.07 (m, 2H), 11.39 (s, |
| | | | | 1H). |
| | | | | Four protons are not apparent. |
| | | | | ¹⁹F NMR (376 MHz, DMSO-d6): δ ppm |
| | | | | −146.65 |
| P-332 | A-57 | C-111d | 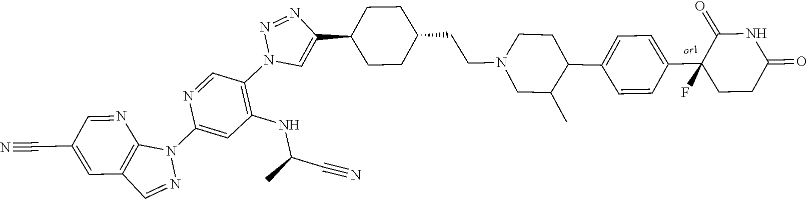 [SMILES: CC1CN(CC[C@H]2CC[C@H](c3cn(-c4cnc(-n5ncc6cc(C#N)cnc65)cc4N[C@H](C)C#N)nn3)CC2)CCC1c1ccc([C@@]2(F)CCC(=O)NC2=O)cc1] | 11.9% yield. LCMS method 13, retention time: 2.67 min, 96.8% purity at 220 nm, [M + H]⁺ = 769.4 ¹H NMR (400 MHz, DMSO-d6): δ ppm 0.74 (d, J = 6.8 Hz, 3H), 1.18 - 1.30 (m, 2H), 1.42 - 1.68 (m, 3H), 1.67 (d, J = 6.8 Hz, 3H), 1.70 - 1.74 (m, 2H), 1.92 - 1.99 (m, 4H), 2.15 - 2.22 (m, 3H), 2.35 - 2.45 (m, 1H), 2.70 - 2.85 (m, 4H), 3.20 - 3.23 (m, 2H), 3.55 - 3.68 (m, 2H), 4.93 - 4.97 (m, 1H), 7.06 (d, J = 8.0 Hz, 1H), 7.32 (d, J = 8.0 Hz, 2H), 7.47 (d, J = 8.4 Hz, 2H), 7.48 (s, 1H), 8.31 (s, 1H), 8.46 (s, 1H), 8.66 (s, 1H), 8.97 (d, J = 2.0 Hz, 1H), |
| | | | | 9.01 (d, J = 2.0 Hz, 1H), 11.01 (s, 1H). |
| | | | | Three protons are not apparent. |
| | | | | ¹⁹F NMR (376 MHz, DMSO-d6): δ ppm |
| | | | | −146.439 |
| P-333 | A-57 | C-111c | 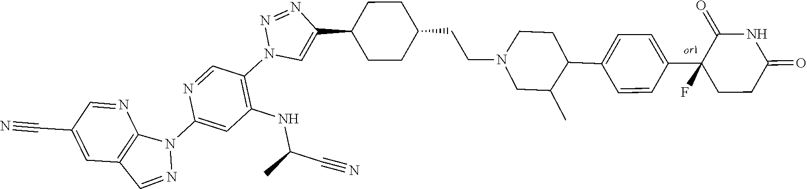 [SMILES: CC1CN(CC[C@H]2CC[C@H](c3cn(-c4cnc(-n5ncc6cc(C#N)cnc65)cc4N[C@H](C)C#N)nn3)CC2)CCC1c1ccc([C@@]2(F)CCC(=O)NC2=O)cc1] | 17.5% yield. LCMS method 7, retention time: 1.86 min, 93.4% purity at 220 nm, [M + H]⁺ = 769.2 ¹H NMR (400 MHz, DMSO-d6): δ ppm 0.68 (d, J = 6.8 Hz, 3H), 1.13 - 1.23 (m, 2H), 1.40 - 1.67 (m, 9H), 1.87 - 1.93 (m, 4H), 2.06 - 2.18 (m, 3H), 2.68 - 2.80 (m, 2H), 2.98 - 3.03 (m, 1H), 3.15 - 3.21 (m, 2H), 3.56 - 3.61 (m, 2H), 4.97 - 5.03 (m, 1H), 7.19 (d, J = 8.0 Hz, 1H), 7.30 (d, J = 8.4 Hz, 2H), 7.44 (d, J = 8.0 Hz, 2H), 7.68 (s, 1H), 8.37 - 8.43 (m, 2H), 8.72 (s, 1H), 9.06 - 9.07 (m, 2H), 11.39 (s, |
| | | | | 1H). |
| | | | | Four protons are not apparent. |
| | | | | ¹⁹F NMR (376 MHz, DMSO-d6): δ ppm |
| | | | | −146.65 |
| P-337 | A-57 | C-46 | 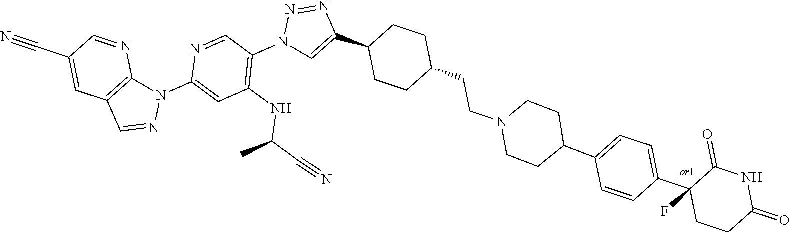 [SMILES: C[C@H](C#N)Nc1cc(-n2ncc3cc(C#N)cnc32)ncc1-n1cc([C@H]2CC[C@H](CCN3CCC(c4ccc([C@@]5(F)CCC(=O)NC5=O)cc4)CC3)CC2)nn1] | 47.1% yield. LCMS method 12, retention time: 1.39 min, 100% purity at 220 nm, [M + H]⁺ = 755.1 ¹H NMR (500 MHz, DMSO-d6) δ 11.50 - 11.23 (m, 1H), 9.33 - 9.15 (m, 1H), 9.10 - 9.03 (m, 2H), 8.72 (s, 1H), 8.41 (s, 1H), 8.37 (s, 1H), 7.67 (s, 1H), 7.48 - 7.40 (m, 2H), 7.35 (d, J = 8.1 Hz, 2H), 7.24 - 7.16 (m, 1H), 7.13 - 6.97 (m, 1H), 5.05 - 4.98 (m, 1H), 3.63 (br d, J = 11.9 Hz, 1H), 3.41 (br d, J = 7.7 Hz, 2H), 3.17 (br s, 1H), 3.13 - 3.00 (m, 2H), 2.94 - 2.84 (m, 1H), 2.81 - 2.62 (m, 3H), 2.54 - 2.47 (m, 21H), 2.37 - 2.27 (m, 1H), 2.16 (br d, J = 11.5 Hz, 2H), 2.11 - 1.97 (m, 2H), 1.88 (br s, 4H), 1.68 - 1.58 (m, 5H), 1.57 - 1.44 (m, |
| | | | | 2H), 1.44 - 1.36 (m, 1H), 1.26 - 1.13 (m, 2H) |
| | | | | ¹⁹F NMR (471 MHz, DMSO-d6) δ −147.19 (s, |
| | | | | 1F) |
| P-338 | A-57 | C-46i | 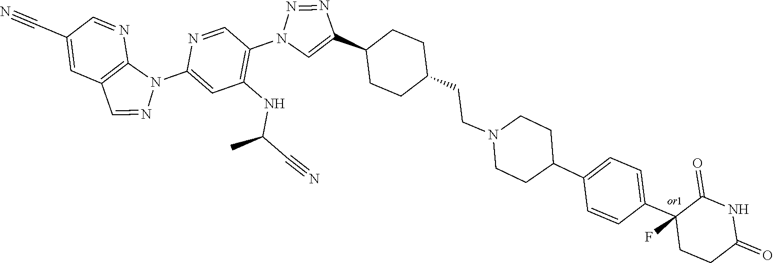 [SMILES: C[C@H](C#N)Nc1cc(-n2ncc3cc(C#N)cnc32)ncc1-n1cc([C@H]2CC[C@H](CCN3CCC(c4ccc([C@@]5(F)CCC(=O)NC5=O)cc4)CC3)CC2)nn1] | 63.2% yield. LCMS method 12, retention time: 1.41 min, 100% purity at 220 nm, [M + H]⁺ = 755.1 ¹H NMR (500 MHz, DMSO-d6) δ 11.39 (s, 1H), 9.08 - 9.03 (m, 2H), 8.72 (s, 1H), 8.41 (s, 1H), 8.36 (s, 1H), 7.67 (s, 1H), 7.48 - 7.40 (m, 2H), 7.38 - 7.32 (m, 2H), 7.19 (br d, J = 8.2 Hz, 1H), 5.02 (br t, J = 7.2 Hz, 1H), 3.62 (br d, J = 10.5 Hz, 2H), 3.17 (br s, 1H), 3.12 - 2.98 (m, 2H), 2.89 (br s, 1H), 2.81 - 2.62 (m, 3H), 2.37 - 2.27 (m, 1H), 2.16 (br d, J = 12.4 Hz, 2H), 2.10 - 1.97 (m, 2H), 1.97 - 1.80 (m, 4H), 1.68 - 1.57 (m, 5H), 1.50 (q, J = 12.0 Hz, 2H), 1.44 - 1.35 (m, 1H), 1.18 (q, J = 12.2 Hz, 2H) ¹⁹F NMR (471 MHz, DMSO-d6) δ −147.28 (s, 1F) |
| P-339 | A-57 | C-110 | 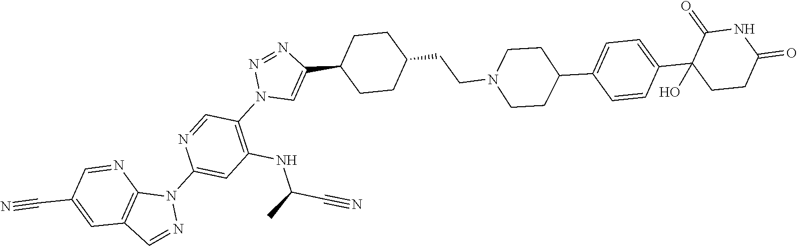 [SMILES: C[C@H](C#N)Nc1cc(-n2ncc3cc(C#N)cnc32)ncc1-n1cc([C@H]2CC[C@H](CCN3CCC(c4ccc(C5(O)CCC(=O)NC5=O)cc4)CC3)CC2)nn1] | 25.9% yield. LCMS method 12, retention time: 1.35 min, 83.3% purity at 220 nm, [M + H]⁺ = 753.2 ¹H NMR (500 MHz, DMSO-d6) δ 9.36 - 9.20 (m, 1H), 9.05 (d, J = 6.0 Hz, 2H), 8.71 (s, 1H), 8.41 (s, 1H), 8.36 (s, 1H), 7.67 (s, 1H), 7.27 - 7.16 (m, 6H), 7.13 (s, 1H), 7.03 (s, 1H), 5.01 (quin, J = 7.2 Hz, 1H), 4.06 (dd, J = 11.4, 4.2 Hz, 1H), 3.62 (br d, J = 11.6 Hz, 2H), 3.17 (s, 1H), 3.12 - 3.00 (m, 2H), 2.89 - 2.72 (m, 3H), 2.72 - 2.62 (m, 1H), 2.55 - 2.53 (m, 1H), 2.22 - 2.09 (m, 3H), 2.08 - 1.96 (m, 3H), 1.88 (br s, 4H), 1.68 - 1.58 (m, 5H), 1.50 (q, J = 12.6 Hz, 2H), 1.44 - 1.35 (m, 1H), 1.26 - 1.13 (m, 2H) |
| P-349 | A-57 | C-109 | 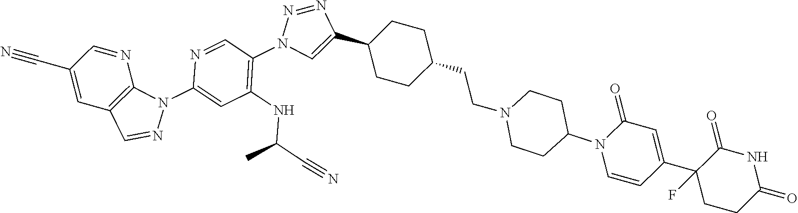 [SMILES: C[C@H](C#N)Nc1cc(-n2ncc3cc(C#N)cnc32)ncc1-n1cc([C@H]2CC[C@H](CCN3CCC(n4ccc(C5(F)CCC(=O)NC5=O)cc4=O)CC3)CC2)nn1] | 5.8% yield. LCMS method 12, retention time: 1.46 min, 83.3% purity at 220 nm, [M + H]⁺ = 772.2 ¹H NMR (500 MHz, DMSO-d6) δ 9.06 (dd, J = 9.0, 1.9 Hz, 2H), 8.72 (s, 1H), 8.41 (s, 1H), 8.36 (s, 1H), 8.24 - 8.17 (m, 1H), 7.68 (s, 1H), 7.20 (d, J = 8.3 Hz, 1H), 7.03 (br t, J = 6.3 Hz, 1H), 6.85 - 6.78 (m, 1H), 5.14 - 5.06 (m, 1H), 5.02 (br t, J = 7.3 Hz, 1H), 3.48 (br s, 1H), 2.81 - 2.67 (m, 2H), 2.44 - 2.27 (m, 1H), 2.17 - 1.99 (m, 3H), 1.93 - 1.85 (m, 3H), 1.85 - 1.70 (m, 1H), 1.62 (d, J = 7.0 Hz, 3H), 1.54 - 1.43 (m, 3H), 1.43 - 1.33 (m, 1H), 1.19 - |
| | | | | 1.09 (m, 2H) |
| | | | | ¹⁹F NMR (471 MHz, DMSO-d6) δ −157.16 |
| | | | | (br s, 1F) |
| P-350 | A-57 | C-107 | 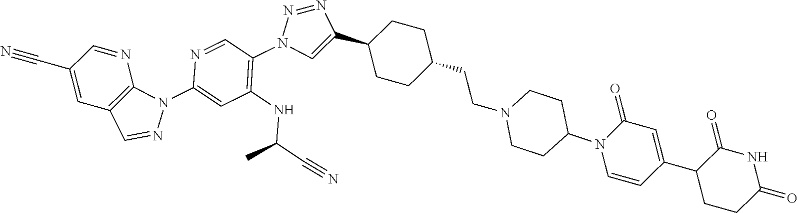 [SMILES: C[C@H](C#N)Nc1cc(-n2ncc3cc(C#N)cnc32)ncc1-n1cc([C@H]2CC[C@H](CCN3CCC(n4ccc(C5CCC(=O)NC5=O)cc4=O)CC3)CC2)nn1] | 13.2% yield. LCMS method 12, retention time: 1.33 min, 95.8% purity at 220 nm, [M + H]⁺ = 754.3 ¹H NMR (500 MHz, DMSO-d6) δ 9.07 - 9.03 (m, 2H), 8.71 (s, 1H), 8.40 (s, 1H), 8.35 (s, 1H), 8.08 (d, J = 5.1 Hz, 1H), 7.67 (s, 1H), 7.19 (br d, J = 8.2 Hz, 1H), 6.85 (br d, J = 5.1 Hz, 1H), 6.66 (s, 1H), 5.08 - 4.97 (m, 2H), 3.91 - 3.86 (m, 3H), 3.17 (d, J = 5.0 Hz, 3H), 2.77 - 2.59 (m, 2H), 2.31 - 2.18 (m, 1H), 2.13 (br d, J = 12.2 Hz, 2H), 2.03 (br s, 2H), 1.86 (br d, J = 11.4 Hz, 2H), 1.72 (br s, 2H), 1.61 (d, J = 7.0 Hz, 3H), 1.54 - 1.41 (m, 4H), 1.41 - |
| | | | | 1.31 (m, 1H), 1.18 - 1.08 (m, 2H) |
| P-352 | A-57 | C-108 | 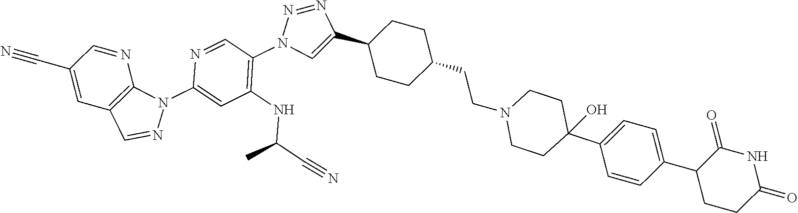 [SMILES: C[C@H](C#N)Nc1cc(-n2ncc3cc(C#N)cnc32)ncc1-n1cc([C@H]2CC[C@H](CCN3CCC(O)(c4ccc(C5CCC(=O)NC5=O)cc4)CC3)CC2)nn1] | 37.6% yield. LCMS method 12, retention time: 1.30 min, 100% purity at 220 nm, [M + H]⁺ = 753.3 ¹H NMR (500 MHz, DMSO-d6) δ 10.84 (s, 1H), 9.26 (br s, 1H), 9.06 (d, J = 6.4 Hz, 2H), 8.72 (s, 1H), 8.41 (s, 1H), 8.37 (s, 1H), 7.68 (s, 1H), 7.43 (d, J = 8.1 Hz, 2H), 7.27 - 7.18 (m, 3H), 5.02 (t, J = 7.3 Hz, 1H), 3.91 - 3.84 (m, 1H), 3.33 - 3.20 (m, 2H), 2.81 - 2.63 (m, 2H), 2.25 - 2.13 (m, 4H), 2.09 - 1.97 (m, 1H), 1.95 - 1.83 (m, 4H), 1.70 - 1.59 (m, 5H), 1.58 - 1.44 (m, 2H), 1.44 - 1.36 (m, 1H), 1.26 - 1.14 (m, 2H) |
| P-390 | A-57 | C-111a | 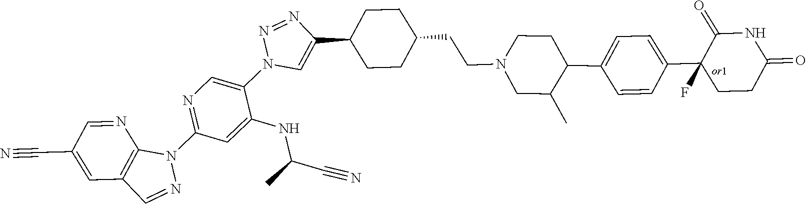 [SMILES: CC1CN(CC[C@H]2CC[C@H](c3cn(-c4cnc(-n5ncc6cc(C#N)cnc65)cc4N[C@H](C)C#N)nn3)CC2)CCC1c1ccc([C@@]2(F)CCC(=O)NC2=O)cc1] | 33.4% yield. LCMS method 7, retention time: 1.99 min, 92.8% purity at 220 nm, [M + H]⁺ = 769.4 ¹H NMR (400 MHz, DMSO-d6): δ ppm 0.68 (d, J = 6.4 Hz, 3H), 1.21 - 1.25 (m, 2H), 1.38 - 1.52 (m, 4H), 1.61 (d, J = 6.8 Hz, 3H), 1.63 - 1.78 (m, 2H), 1.90 - 1.93 (m, 4H), 2.11 - 2.18 (m, 3H), 2.26 - 2.31 (m, 1H), 2.71 - 2.78 (m, 4H), 2.98 - 3.06 (m, 1H), 3.17 - 3.19 (m, 2H), 3.30 - 3.42 (m, 1H), 3.54 - 3.60 (m, 2H), 4.95 - 5.06 (m, 1H), 7.19 (d, J = 7.6 Hz, 1H), 7.30 (d, J = 8.0 Hz, 2H), 7.44 (d, J = 8.4 Hz, 2H), 7.68 (s, 1H), 8.37 (s, 1H), 8.41 (s, 1H), |
| | | | | 8.72 (s, 1H), 9.05 - 9.07 (m, 2H), 11.39 (s, |
| | | | | 1H). |
| | | | | Two protons are not apparent. |
| | | | | ¹⁹F NMR (376 MHz, DMSO-d6): δ ppm |
| | | | | −146.4 |
| P-413 | A-57 | C-116 | 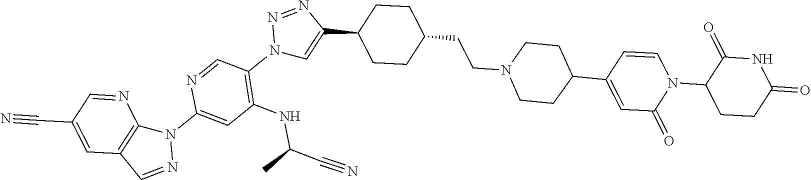 [SMILES: C[C@H](C#N)Nc1cc(-n2ncc3cc(C#N)cnc32)ncc1-n1cc([C@H]2CC[C@H](CCN3CCC(c4ccn(C5CCC(=O)NC5=O)c(=O)c4)CC3)CC2)nn1] | 17.7% yield as an off white solid. LCMS method 12, retention time: 1.51 min, 96.2% purity at 220 nm, [M + H]⁺ = 754.4 ¹H NMR (400 MHz, DMSO-d6): δ ppm 1.12 - 1.22 (m, 2H), 1.37 - 1.41 (m, 1H), 1.46 - 1.56 (m, 2H), 1.62 (d, J = 6.8 Hz, 3H), 1.65 - 1.67 (m, 2H), 1.70 - 1.81 (m, 2H), 1.88 - 1.91 (m, 2H), 1.95 - 2.00 (m, 1H), 2.06 - 2.09 (m, 2H), 2.15 - 2.18 (m, 2H), 2.57 - 2.61 (m, 1H), 2.70 - 2.85 (m, 3H), 2.98 - 3.07 (m, 2H), 3.14 - 3.20 (m, 2H), 3.29 - 3.35 (m, 1H), 3.61 - 3.64 (m, 2H), 4.99 - 5.05 (m, |
| | | | | 1H), 5.38 (s, 1H), 6.22 - 6.26 (m, 2H), 7.20 |
| | | | | (d, J = 8.4 Hz, 1H), 7.62 (d, J = 7.2 Hz, 1H), |
| | | | | 7.68 (s, 1H), 8.37 (s, 1H), 8.42 (s, 1H), 8.73 |
| | | | | (s, 1H), 9.06 - 9.08 (m, 2H), 11.02 (s, 1H). |
| P-417 | A-57 | C-115b | 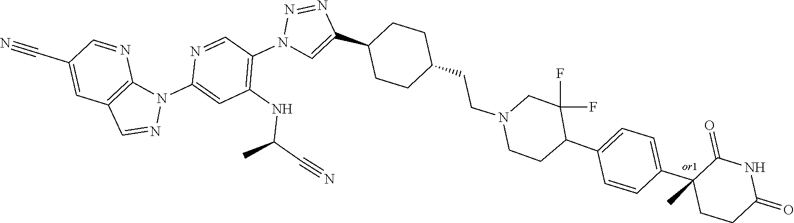 [SMILES: C[C@H](C#N)Nc1cc(-n2ncc3cc(C#N)cnc32)ncc1-n1cc([C@H]2CC[C@H](CCN3CCC(c4ccc([C@@]5(C)CCC(=O)NC5=O)cc4)C(F)(F)C3)CC2)nn1] | 32.2% yield as a white solid. LCMS method 12, retention time: 2.37 min, 94.3% purity at 220 nm, [M + H]⁺ = 786.6 ¹H NMR (400 MHz, DMSO-d6): δ ppm 1.12 - 1.24 (m, 2H), 1.40 - 1.58 (m, 6H), 1.61 (d, J = 6.8 Hz, 3H), 1.66 - 1.68 (m, 2H), 1.87 - 1.90 (m, 2H), 2.04 - 2.18 (m, 5H), 2.32 - 2.39 (m, 1H), 2.74 - 2.82 (m, 1H), 3.16 - 3.30 (m, 3H), 3.45 - 3.70 (m, 3H), 4.98 - 5.05 (m, 1H), 7.19 (d, J = 8.0 Hz, 1H), 7.33 (s, 4H), 7.68 (s, 1H), 8.37 (s, 1H), 8.41 (s, 1H), 8.72 (s, 1H), 9.05 - 9.07 (m, 2H), 10.94 (s, 1H). Three protons are not apparent. ¹⁹F NMR (376 MHz, DMSO-d6): δ ppm |
| | | | | −101.653 (d, 2J_{F} = 254.9 Hz), −109.067 (d, 2J_{F} = |
| | | | | 247.8 Hz) |
| P-424 | A-57 | C-114 | 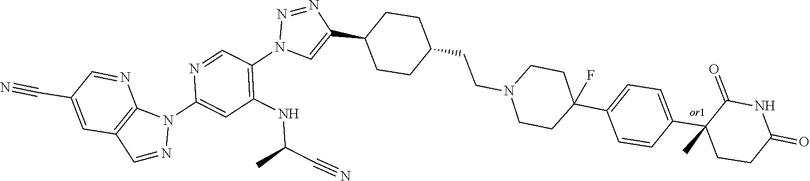 [SMILES: C[C@H](C#N)Nc1cc(-n2ncc3cc(C#N)cnc32)ncc1-n1cc([C@H]2CC[C@H](CCN3CCC(F)(c4ccc([C@@]5(C)CCC(=O)NC5=O)cc4)CC3)CC2)nn1] | 8.7% yield as an off white solid. LCMS method 7, retention time: 1.95 min, 97.5% purity at 220 nm, [M + H]⁺ = 769.4 ¹H NMR (400 MHz, DMSO-d6): δ ppm 1.11 - 1.22 (m, 4H), 1.33 (s, 1H), 1.46 (s, 3H), 1.49 - 1.55 (m, 2H), 1.61 (d, J = 7.2 Hz, 3H), 1.62 - 1.70 (m, 2H), 1.89 - 1.91 (m, 2H), 2.10 - 2.18 (m, 3H), 2.23 - 2.29 (m, 3H), 2.36 - 2.42 (m, 2H), 2.72 - 2.80 (m, 1H), 3.21 - 3.30 (m, 4H), 3.58 - 3.61 (m, 2H), 4.99 - 5.05 (m, 1H), 7.19 (d, J = 8.4 Hz, 1H), 7.38 - 7.44 (m, 4H), 7.68 (s, 1H), 8.37 (s, 1H), 8.41 (s, 1H), |
| | | | | 8.72 (s, 1H), 9.06 - 9.07 (m, 2H), 10.95 (s, |
| | | | | 1H). |
| | | | | ¹⁹F NMR (376 MHz, DMSO-d6): δ ppm |
| | | | | −156.47 |
| P-513 | A-113 | C-46 | 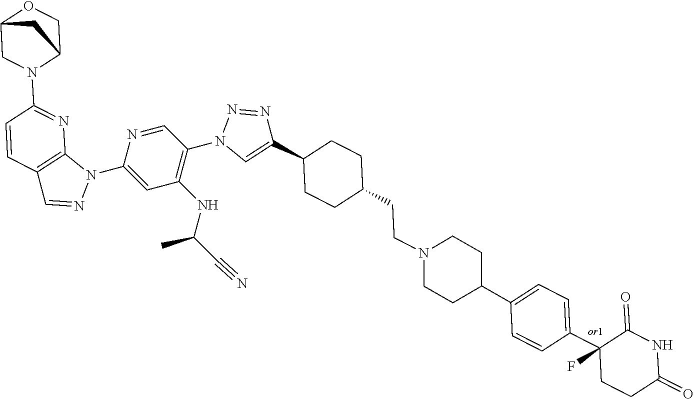 [SMILES: C[C@H](C#N)Nc1cc(-n2ncc3ccc(N4C[C@H]5C[C@@H]4CO5)nc32)ncc1-n1cc([C@H]2CC[C@H](CCN3CCC(c4ccc([C@@]5(F)CCC(=O)NC5=O)cc4)CC3)CC2)nn1] | 4.1% yield as a pale brown solid. LCMS method 7, retention time: 1.66 min, 86.2% purity at 220 nm, [M + H]⁺ = 827.3 ¹H NMR (400 MHz, DMSO-d6): δ ppm 1.11 - 1.22 (m, 2H), 1.38 - 1.50 (m, 3H), 1.63 (s, 5H), 1.81 - 1.94 (m, 7H), 2.02 - 2.08 (m, 2H), 2.11 - 2.15 (m, 2H), 2.39 - 2.46 (m, 1H), 2.62 - 2.77 (m, 3H), 2.86 - 3.92 (m, 1H), 3.01 - 3.09 (m, 2H), 3.13 - 3.19 (m, 2H), 3.59 - 3.63 (m, 2H), 3.81 (brs, 2H), 4.73 (s, 1H), 5.05 (brs, 1H), 7.34 - 7.36 (m, 2H), 7.42 - 7.48 (m, 2H), 7.91 - 7.98 (m, 2H), 8.30 - 8.39 (m, 2H), 11.39 (s, 1H). Six protons are not apparent. ¹⁹F NMR (376 MHz, DMSO-d6): δ ppm −146.4 |

### Example S76. Synthesis of 1-(4-(piperidin-4-yl)phenyl)dihydropyrimidine-2,4(1H,3H)-dione (C-119)

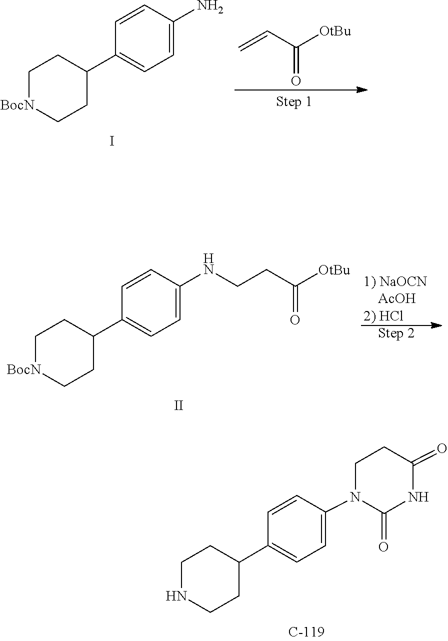

Step 1: Preparation of 4-(4-((3-(tert-butoxy)-3-oxopropyl)amino)phenyl)piperidine-1-carboxylate (II). To a scintillation vial was added 1 g of Al₂O₃, tert-butyl 4-(4-aminophenyl)piperidine-1-carboxylate (0.138 g, 0.5 mmol) (I), tert-butyl acrylate (0.067 ml, 0.455 mmol), and a large stir bar. The vial was sealed and placed under nitrogen atmosphere. Reaction mixture as then heated to 65° C. and stirred overnight. After this time, the solids were washed with ethyl acetate and filtered over celite plug. The organic eluent was coated onto silica gel and purified via column chromatography in 0>100% ethyl acetate in heptanes to afford tert-butyl 4-(4-((3-(tert-butoxy)-3-oxopropyl)amino)phenyl)piperidine-1-carboxylate as an off white solid (II, 140 mg, 76% yield). [M+H]⁺=405.2.

Step 2: Preparation of 1-(4-(piperidin-4-yl)phenyl)dihydropyrimidine-2,4(1H,3H)-dione (C-119). A 20 mL vial was charged with tert-butyl 4-(4-((3-(tert-butoxy)-3-oxopropyl)amino)phenyl)piperidine-1-carboxylate (II, 101 mg, 0.25 mmol), sodium cyanate (81 mg, 1.25 mmol), THF (2.5 mL), and acetic acid (6.5 mL). Reaction mixture was placed under nitrogen atmosphere and stirred at RT overnight. The crude mixture was then diluted in saturated sodium bicarbonate solution. This aqueous mixture was then extracted 3× with dichloromethane. Organic layers were then combined and dried with brine and magnesium sulfate before being concentrated in vacuo. This residue was then dissolved in 1 M hydrochloric acid in dioxane and stirred overnight at 90° C. Volatiles and remaining acid were then removed under nitrogen stream. This residue was then purified via reverse phase preparatory HPLC in acetonitrile in water with 0.1% trifluoroacetic acid modifier to furnish 1-(4-(piperidin-4-yl)phenyl)dihydropyrimidine-2,4(1H,3H)-dione as an amorphous solid (C-119, 27 mg, 39% yield). [M+H]⁺=274.3.

Table 63 summarizes the intermediates that were synthesized using a procedure similar to Example S76.

TABLE 63
Intermediate Compounds Prepared via a Procedure Similar
to Example S76.

| Intermediate | |
|---|---|
| No. | Structure |
| C-119 | 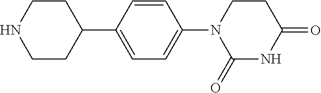 [SMILES: O=C1CCN(c2ccc(C3CCNCC3)cc2)C(=O)N1] |
| C-134 | 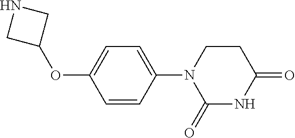 [SMILES: O=C1CCN(c2ccc(OC3CNC3)cc2)C(=O)N1] |
| C-138 | 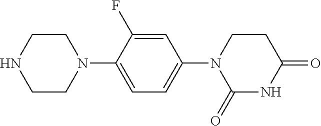 [SMILES: O=C1CCN(c2ccc(N3CCNCC3)c(F)c2)C(=O)N1] |

### Example S77. Synthesis of of (R)-1-(6-(2-methylpiperazin-1-yl)pyridin-3-yl)dihydropyrimidine-2,4(1H,3H)-dione (C-150)

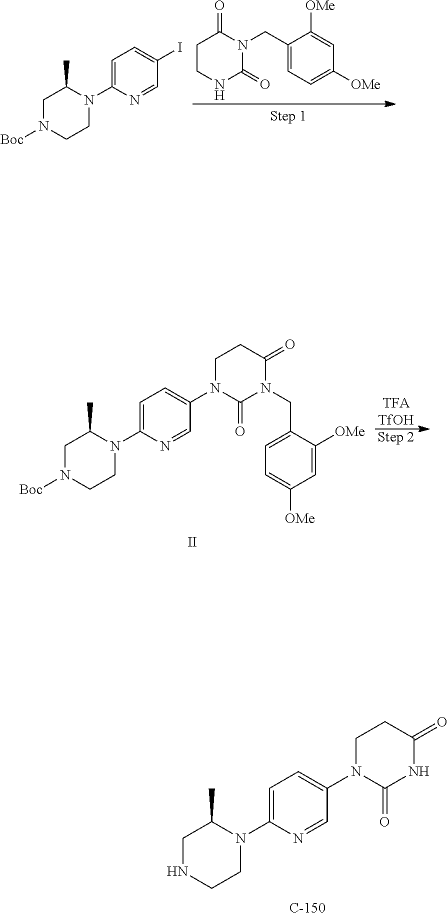

Step 1: Preparation of tert-butyl-(R)-4-(5-(3-(2,4-dimethoxybenzyl)-2,4-dioxotetrahydropyrimidin-1(2H)-yl)pyridin-2-yl)-3-methylpiperazine-1-carboxylate (II). To a scintillation vial was added cuprous iodide (21.25 mg, 0.112 mmol), tripotassium phosphate (47.4 mg, 0.223 mmol), 3-(2,4-dimethoxybenzyl)dihydropyrimidine-2,4(1H,3H)-dione (19.66 mg, 0.074 mmol tert-butyl (R)-4-(5-iodopyridin-2-yl)-3-methylpiperazine-1-carboxylate (I, 30.0 mg, 0.074 mmol), and 1,4-dioxane (750 μl). The vial was sealed and the atmosphere was evacuated and purged with nitrogen 3×. Reaction mixture was then sparged with nitrogen while stirring at room temperature for 10 mins. After this time, (1S,2S)-cyclohexane-1,2-diamine (8.50 mg, 0.074 mmol) was added quickly and sparging was continued until green color evolved. The vent needle was then removed and the reaction mixture was heated to 90° C. with overnight stirring. The crude reaction was removed from the heat and allowed to cool to room temperature before filtration. The filtrate was then purified via reverse phase preparatory HPLC in 5>95% acetonitrile in water with 0.1% trifluoroacetic acid modifier to furnish tert-butyl (R)-4-(5-(3-(2,4-dimethoxybenzyl)-2,4-dioxotetrahydropyrimidin-1(2H)-yl)pyridin-2-yl)-3-methylpiperazine-1-carboxylate as an oily residue (II, 38 mg, 95% yield). [M+H]⁺=484.5.

Step 2: Preparation of (R)-1-(6-(2-methylpiperazin-1-yl)pyridin-3-yl)dihydropyrimidine-2,4(1H,3H)-dione (C-150). To a vial containing tert-butyl (R)-4-(5-(3-(2,4-dimethoxybenzyl)-2,4-dioxotetrahydropyrimidin-1(2H)-yl)pyridin-2-yl)-3-methylpiperazine-1-carboxylate (II, 38 mg, 0.070 mmol) was added 0.3 mL neat trifluoroacetic acid and 0.3 mL triflic acid. This was reaction mix was then stirred at room temperature for 3 h. Volatiles were then removed under nitrogen stream overnight to afford (R)-1-(6-(2-methylpiperazin-1-yl)pyridin-3-yl)dihydropyrimidine-2,4(1H,3H)-dione as a purple oil (C-150, quantitative yield). [M+H]⁺=290.3.

Table 64 summarizes the intermediates that were synthesized using a procedure similar to Example S77.

TABLE 64
Intermediate Compounds Prepared via a Procedure Similar
to Example S77.

| Intermediate | |
|---|---|
| No. | Structure |
| C-131 | 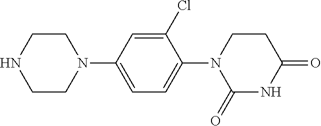 [SMILES: O=C1CCN(c2ccc(N3CCNCC3)cc2Cl)C(=O)N1] |
| C-133 | 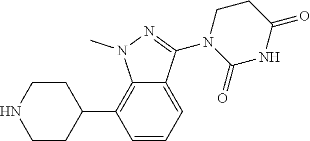 [SMILES: Cn1nc(N2CCC(=O)NC2=O)c2cccc(C3CCNCC3)c21] |
| C-139 | 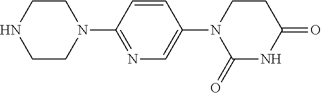 [SMILES: O=C1CCN(c2ccc(N3CCNCC3)nc2)C(=O)N1] |
| C-141 | 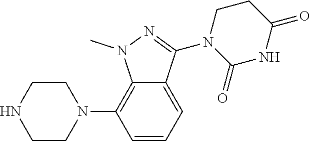 [SMILES: Cn1nc(N2CCC(=O)NC2=O)c2cccc(N3CCNCC3)c21] |
| C-142 | 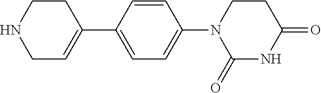 [SMILES: O=C1CCN(c2ccc(C3=CCNCC3)cc2)C(=O)N1] |
| C-143 | 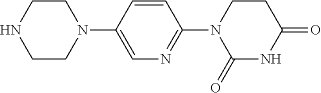 [SMILES: O=C1CCN(c2ccc(N3CCNCC3)cn2)C(=O)N1] |
| C-145 | 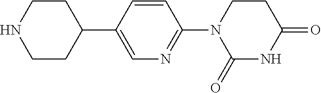 [SMILES: O=C1CCN(c2ccc(C3CCNCC3)cn2)C(=O)N1] |
| C-150 | 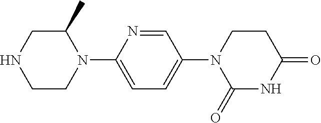 [SMILES: C[C@@H]1CNCCN1c1ccc(N2CCC(=O)NC2=O)cn1] |
| C-151 | 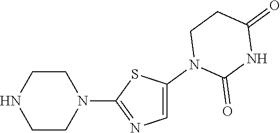 [SMILES: O=C1CCN(c2cnc(N3CCNCC3)s2)C(=O)N1] |
| C-152 | 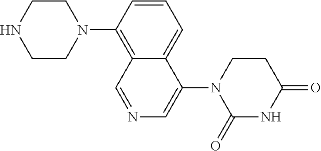 [SMILES: O=C1CCN(c2cncc3c(N4CCNCC4)cccc23)C(=O)N1] |
| C-153 | 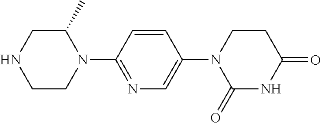 [SMILES: C[C@H]1CNCCN1c1ccc(N2CCC(=O)NC2=O)cn1] |
| C-154 | 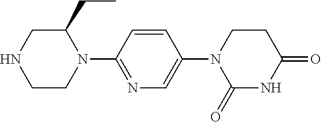 [SMILES: CC[C@@H]1CNCCN1c1ccc(N2CCC(=O)NC2=O)cn1] |
| C-160 | 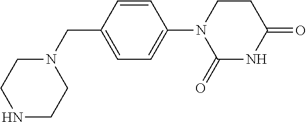 [SMILES: O=C1CCN(c2ccc(CN3CCNCC3)cc2)C(=O)N1] |
| C-163 | 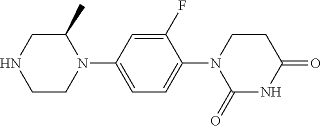 [SMILES: C[C@@H]1CNCCN1c1ccc(N2CCC(=O)NC2=O)c(F)c1] |
| C-164 | 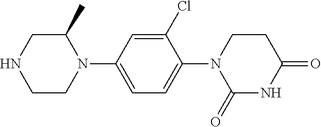 [SMILES: C[C@@H]1CNCCN1c1ccc(N2CCC(=O)NC2=O)c(Cl)c1] |
| C-165 | 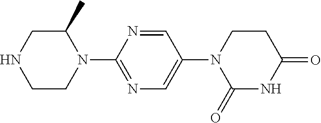 [SMILES: C[C@@H]1CNCCN1c1ncc(N2CCC(=O)NC2=O)cn1] |
| C-167 | 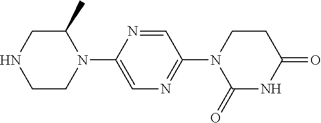 [SMILES: C[C@@H]1CNCCN1c1cnc(N2CCC(=O)NC2=O)cn1] |
| C-168 | 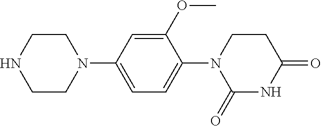 [SMILES: COc1cc(N2CCNCC2)ccc1N1CCC(=O)NC1=O] |
| C-170 | 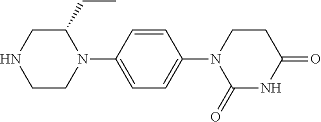 [SMILES: CC[C@H]1CNCCN1c1ccc(N2CCC(=O)NC2=O)cc1] |

### Example S78. Synthesis of 1-(4-(piperazin-1-yl)phenyl)dihydropyrimidine-2,4(1H,3H)-dione (C-118)

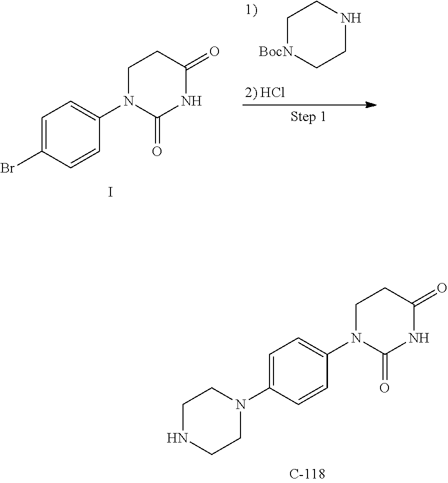

Step 1: Preparation of 1-(4-(piperazin-1-yl)phenyl)dihydropyrimidine-2,4(1H,3H)-dione (C-118). To a microwave vial was added 1-(4-bromophenyl)dihydropyrimidine-2,4(1H,3H)-dione (I, 100.0 mg, 0.372 mmol), sodium tert-butoxide (71.4 mg, 0.743 mmol), 1-boc-piperazine (69.2 mg, 0.372 mmol), XPhos Pd G4 (32.0 mg, 0.037 mmol), and toluene (3.5 mL). The vial was then sealed with a rubber septum and the atmosphere was evacuated and purged with dinitrogen 3×. The solution was then sparged with dinitrogen while sonicated, then quickly sealed. This sealed vial was then irradiated in a microwave at 160° C. for 10 min. This crude reaction mix was then dry coated onto silica gel under reduce pressure and purified in a gradient elution system of 0>100% ethyl acetate in heptanes. Relevant fractions were combined and concentrated under reduced pressure then dissolved in a solution of hydrochloric acid in dioxane (4 mL, 1 M, 4.0 mmol) and stirred at RT for 2 h. Volatiles and additional acid were removed under nitrogen stream to yield 1-(4-(piperazin-1-yl)phenyl)dihydropyrimidine-2,4(1H,3H)-dione as an amorphous solid (C-118, 84 mg, 83% yield). [M+H]⁺=275.3.

Table 65 summarizes the intermediates that were synthesized using a procedure similar to Example 78.

TABLE 65
Intermediate Compounds Prepared via a Procedure Similar
to Example 78.

| Intermediate | |
|---|---|
| No. | Structure |
| C-118 | 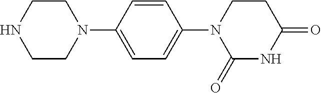 [SMILES: O=C1CCN(c2ccc(N3CCNCC3)cc2)C(=O)N1] |
| C-120 | 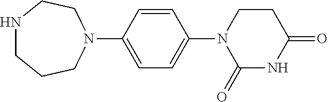 [SMILES: O=C1CCN(c2ccc(N3CCCNCC3)cc2)C(=O)N1] |
| C-121 | 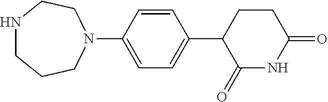 [SMILES: O=C1CCC(c2ccc(N3CCCNCC3)cc2)C(=O)N1] |
| C-128 | 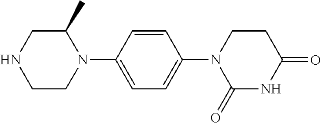 [SMILES: C[C@@H]1CNCCN1c1ccc(N2CCC(=O)NC2=O)cc1] |
| C-130 | 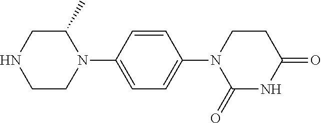 [SMILES: C[C@H]1CNCCN1c1ccc(N2CCC(=O)NC2=O)cc1] |
| C-149 | 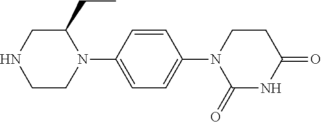 [SMILES: CC[C@@H]1CNCCN1c1ccc(N2CCC(=O)NC2=O)cc1] |
| C-159 | 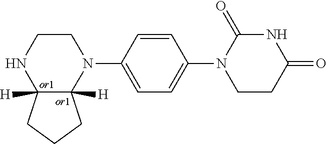 [SMILES: O=C1CCN(c2ccc(N3CCN[C@H]4CCC[C@H]43)cc2)C(=O)N1] |
| | chiral separation |
| C-166 | 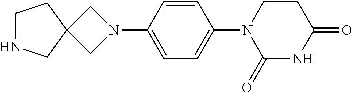 [SMILES: O=C1CCN(c2ccc(N3CC4(CCNC4)C3)cc2)C(=O)N1] |
| C-173 | 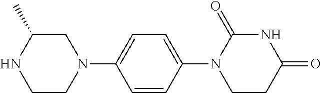 [SMILES: C[C@@H]1CN(c2ccc(N3CCC(=O)NC3=O)cc2)CCN1] |
| C-175 | 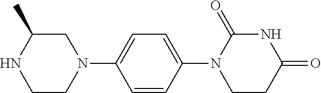 [SMILES: C[C@H]1CN(c2ccc(N3CCC(=O)NC3=O)cc2)CCN1] |
| C-177 | 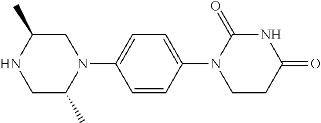 [SMILES: C[C@@H]1CN[C@@H](C)CN1c1ccc(N2CCC(=O)NC2=O)cc1] |
| C-180 | 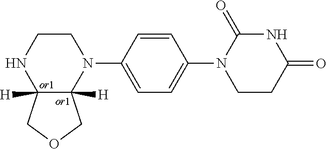 [SMILES: O=C1CCN(c2ccc(N3CCN[C@H]4COC[C@H]43)cc2)C(=O)N1] |
| | chiral separation |
| C-181 | 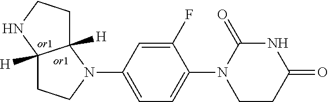 [SMILES: O=C1CCN(c2ccc(N3CC[C@@H]4NCC[C@@H]43)cc2F)C(=O)N1] |
| | chiral separation |
| C-182 | 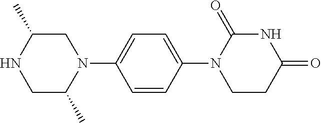 [SMILES: C[C@@H]1CN(c2ccc(N3CCC(=O)NC3=O)cc2)[C@H](C)CN1] |
| C-183 | 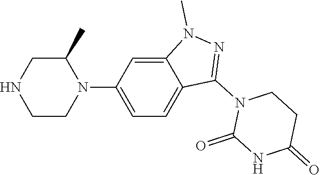 [SMILES: C[C@@H]1CNCCN1c1ccc2c(N3CCC(=O)NC3=O)nn(C)c2c1] |
| C-184 | 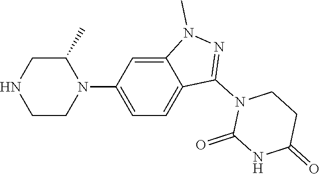 [SMILES: C[C@H]1CNCCN1c1ccc2c(N3CCC(=O)NC3=O)nn(C)c2c1] |
| C-185 | 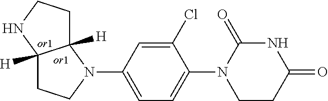 [SMILES: O=C1CCN(c2ccc(N3CC[C@@H]4NCC[C@@H]43)cc2Cl)C(=O)N1] |
| | chiral separation |
| C-186 | 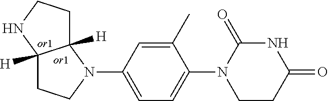 [SMILES: Cc1cc(N2CC[C@@H]3NCC[C@@H]32)ccc1N1CCC(=O)NC1=O] |
| | chiral separation |
| C-187 | 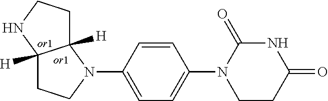 [SMILES: O=C1CCN(c2ccc(N3CC[C@@H]4NCC[C@@H]43)cc2)C(=O)N1] |
| | chiral separation |
| C-188 | 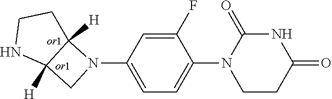 [SMILES: O=C1CCN(c2ccc(N3C[C@@H]4NCC[C@@H]43)cc2F)C(=O)N1] |
| | chiral separation |

### Example S79. Synthesis of 3-((4-(piperazin-1-yl)phenyl)amino)piperidine-2,6-dione (C-122)

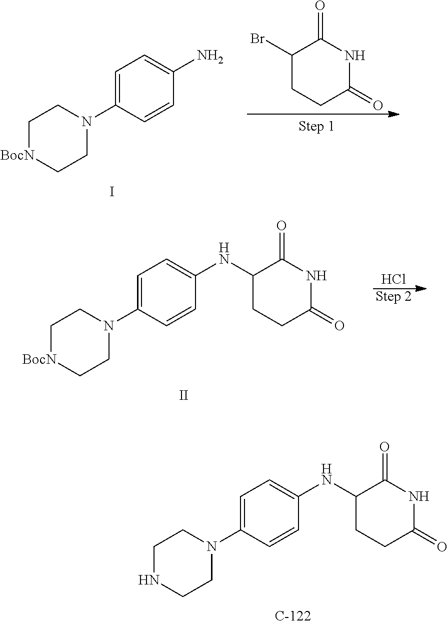

Step 1: Preparation of tert-butyl 4-(4-((2,6-dioxopiperidin-3-yl)amino)phenyl)piperazine-1-carboxylate (II). To a 20 mL vial was added tert-butyl 4-(4-aminophenyl)piperazine-1-carboxylate (I, 60 mg, 0.216 mmol), 3-bromopiperdine-2,6-dione (40 mg, 0.208 mmol), and dioxane (1 mL). This vial was then sealed and placed under nitrogen atmosphere. The crude reaction mixture was then heated to 90° C. and stirred overnight. After this time, the reaction mixture was allowed to cool to room temperature and was then coated onto silica gel under reduced pressure. Purification in a graduated elution system of 0>100% ethyl acetate in heptanes afforded tert-butyl 4-(4-((2,6-dioxopiperidin-3-yl)amino)phenyl)piperazine-1-carboxylate as a green oil (II, 41 mg, 51% yield). [M+H]⁺=389.3.

Step 2: Preparation of 3-((4-(piperazin-1-yl)phenyl)amino)piperidine-2,6-dione (C-122). To a vial containing tert-butyl 4-(4-((2,6-dioxopiperidin-3-yl)amino)phenyl)piperazine-1-carboxylate (II, 41 mg, 0.105 mmol) was added a solution of hydrochloric acid in dioxane (4 mL, 1 M, 0.400 mmol). This solution was stirred at 70° C. for 1 h then allowed to cool to RT. Volatiles were removed under nitrogen stream, affording 3-((4-(piperazin-1-yl)phenyl)amino)piperidine-2,6-dione as an HCl salt (C-122, 34 mg, quantitative yield). [M+H]⁺=289.3.

Table 66 summarizes the intermediates that were synthesized using a procedure similar to Example S79.

TABLE 66
Intermediate Compounds Prepared via a Procedure Similar
to Example S79.

| Intermediate No. | Structure |
|---|---|
| C-122 | 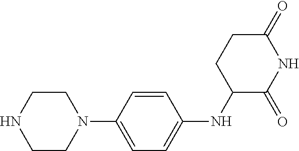 [SMILES: O=C1CCC(Nc2ccc(N3CCNCC3)cc2)C(=O)N1] |
| C-123 | 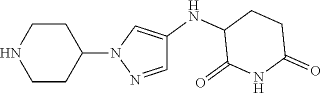 [SMILES: O=C1CCC(Nc2cnn(C3CCNCC3)c2)C(=O)N1] |
| C-125 | 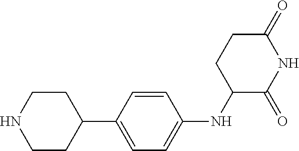 [SMILES: O=C1CCC(Nc2ccc(C3CCNCC3)cc2)C(=O)N1] |

### Example S80. Synthesis of 1-(4-(2-azaspiro[3.3]heptan-6-yl)phenyl)dihydropyrimidine-2,4(1H,3H)-dione (C-147)

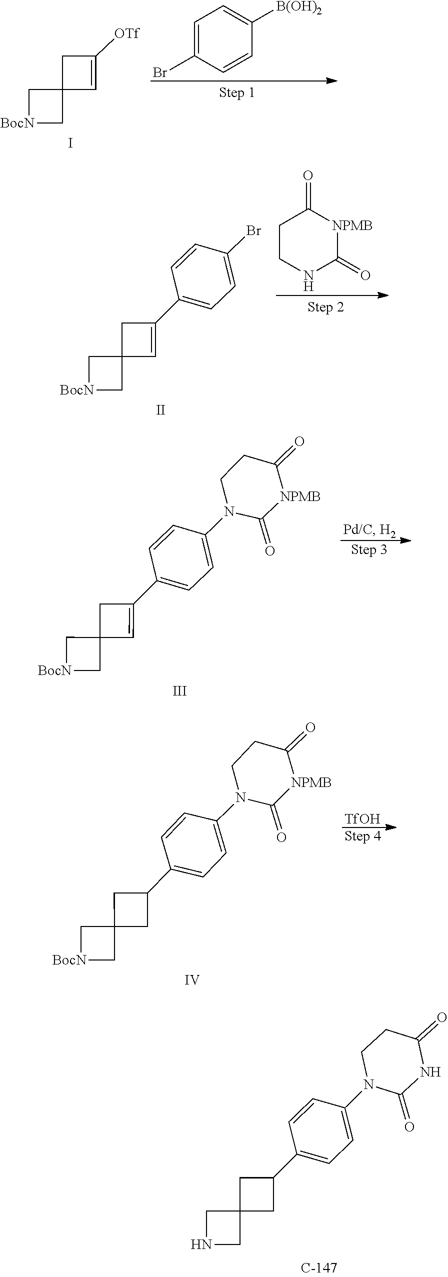

Step 1: Preparation of tert-butyl 6-(4-bromophenyl)-2-azaspiro[3.3]hept-5-ene-2-carboxylate (II). To a stirred solution of tert-butyl 6-(((trifluoromethyl)sulfonyl)oxy)-2-azaspiro[3.3]hept-5-ene-2-carboxylate (I, 1.4 g, 4.08 mmol) in a mixture of 1,4-dioxane (15 mL) and H₂O (2.5 mL) was added (4-bromophenyl)boronic acid (0.901 g, 4.49 mmol) followed by potassium phosphate (1.776 g, 10.19 mmol). The resulting reaction mixture was degassed with dinitrogen for 10 min and then PdCl₂(dppf) (0.298 g, 0.408 mmol) was added. The resulting reaction mixture was stirred at room temperature for 3 h. The reaction mixture was treated with ice-cold water and extracted with EtOAc (2×100 mL). The combined organics were dried over Na₂SO₄ and concentrated under reduced pressure to obtain tert-butyl 6-(4-bromophenyl)-2-azaspiro[3.3]hept-5-ene-2-carboxylate (II, 1.9 g, 84% yield) as a brown gum. The crude was used for the next step without further purification. [M+H-Boc]⁺=250.0.

Step 2: Preparation of tert-butyl6-(4-(3-(4-methoxybenzyl)-2,4-dioxotetrahydropyrimidin-1(2H)-yl)phenyl)-2-azaspiro[3.3]hept-5-ene-2-carboxylate (III). To a stirred solution of tert-butyl 6-(4-bromophenyl)-2-azaspiro[3.3]hept-5-ene-2-carboxylate (II, 1.7 g, 4.85 mmol) in 1,4-dioxane (5 mL) was added 3-(4-methoxybenzyl)dihydropyrimidine-2,4(1H,3H)-dione (1.137 g, 4.85 mmol) followed by potassium phosphate, tribasic (2.57 g, 12.13 mmol). Copper(I) iodide (0.924 g, 4.85 mmol) and N,N-dimethylethylenediamine (0.455 mL, 4.85 mmol) were added to the reaction mixture which was then stirred at 100° C. for 16 h. The reaction mixture was filtered through celite, and the filtrate was concentrated under reduced pressure to obtain crude tert-butyl6-(4-(3-(4-methoxybenzyl)-2,4-dioxotetrahydropyrimidin-1(2H)-yl)phenyl)-2-azaspiro[3.3]hept-5-ene-2-carboxylate (III, 2.1 g, 34.4% yield) as a pale brown gum. The crude was used for the next step without further purification. [M+H-Boc]⁺=404.2.

Step 3: Preparation of tert-butyl 6-(4-(3-(4-methoxybenzyl)-2,4-dioxotetrahydropyrimidin-1(2H)-yl)phenyl)-2-azaspiro[3.3]heptane-2-carboxylate (IV). To a stirred solution of tert-butyl 6-(4-(3-(4-methoxybenzyl)-2,4-dioxotetrahydropyrimidin-1(2H)-yl)phenyl)-2-azaspiro[3.3]hept-5-ene-2-carboxylate (III, 2 2 g, 1.747 mmol) in ethyl acetate (25 mL) was added 10% Pd/C (0.186 g). The resulting reaction mixture was stirred at room temperature under an H₂ atmosphere for 6 h. The reaction mixture was filtered through celite, and the filtrate was concentrated under reduced pressure to obtain crude compound. The crude compound was purified by reverse phase column chromatography to afford tert-butyl 6-(4-(3-(4-methoxybenzyl)-2,4-dioxotetrahydropyrimidin-1(2H)-yl)phenyl)-2-azaspiro[3.3]heptane-2-carboxylate (IV, 700 mg, 66.6% yield) as a brown solid. [M+H-Boc]⁺=406.2.

Step 4: Preparation of 1-(4-(2-azaspiro[3.3]heptan-6-yl)phenyl)dihydropyrimidine-2,4(1H,3H)-dione (C-147). To a stirred solution of tert-butyl 6-(4-(3-(4-methoxybenzyl)-2,4-dioxotetrahydropyrimidin-1(2H)-yl)phenyl)-2-azaspiro[3.3]heptane-2-carboxylate (IV, 280 mg, 0.554 mmol) in TFA (5 mL) was added triflic acid (0.393 mL, 4.43 mmol). The resulting reaction mixture was stirred at 50° C. for 6 h. The reaction mixture was concentrated under reduced pressure to obtain crude residue. The crude residue was triturated with diethyl ether to obtain 1-(4-(2-azaspiro[3.3]heptan-6-yl)phenyl)dihydropyrimidine-2,4(1H,3H)-dione, TFA (C-147, 170 mg, 61.5% yield) as pale brown solid. The crude compound was used for the next step without further purification. [M+H]⁺=286.2.

Table 67 summarizes the intermediates that were synthesized using a procedure similar to Example S80.

TABLE 67
Intermediate Compounds Prepared via a Procedure Similar
to Example S80.

| Intermediate | Structure |
|---|---|
| No. | |
| C-135 | 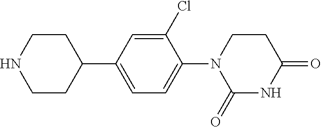 [SMILES: O=C1CCN(c2ccc(C3CCNCC3)cc2Cl)C(=O)N1] |
| C-144 | 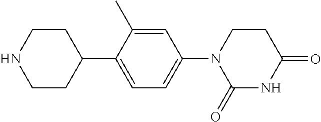 [SMILES: Cc1cc(N2CCC(=O)NC2=O)ccc1C1CCNCC1] |
| C-146 | 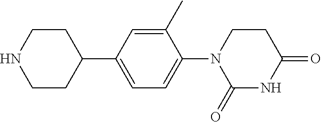 [SMILES: Cc1cc(C2CCNCC2)ccc1N1CCC(=O)NC1=O] |
| C-147 | 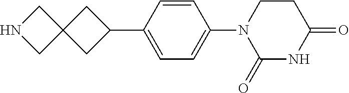 [SMILES: O=C1CCN(c2ccc(C3CC4(CNC4)C3)cc2)C(=O)N1] |
| C-157 | 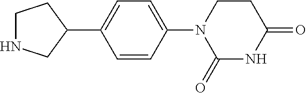 [SMILES: O=C1CCN(c2ccc(C3CCNC3)cc2)C(=O)N1] |
| | chiral separation |
| C-158 | 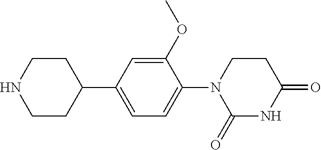 [SMILES: COc1cc(C2CCNCC2)ccc1N1CCC(=O)NC1=O] |
| C-162 | 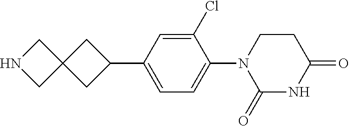 [SMILES: O=C1CCN(c2ccc(C3CC4(CNC4)C3)cc2Cl)C(=O)N1] |
| C-169 | 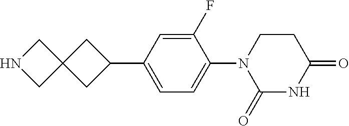 [SMILES: O=C1CCN(c2ccc(C3CC4(CNC4)C3)cc2F)C(=O)N1] |
| C-171 | 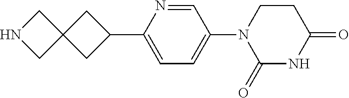 [SMILES: O=C1CCN(c2ccc(C3CC4(CNC4)C3)nc2)C(=O)N1] |

### Example S81. Synthesis of 1-(2-methyl-4-(piperazin-1-yl)phenyl)dihydropyrimidine-2,4(1H,3H)-dione (C-126)

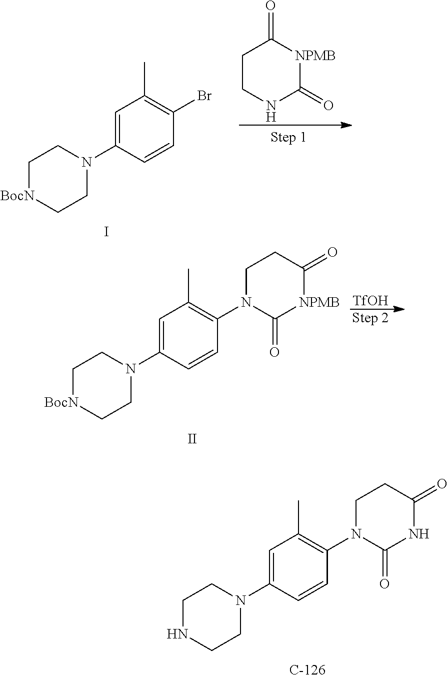

Step 1: Preparation of tert-butyl 4-(4-(3-(4-methoxybenzyl)-2,4-dioxotetrahydropyrimidin-1(2H)-yl)-3-methylphenyl)piperazine-1-carboxylate (II). To a stirred solution of tert-butyl 4-(4-bromo-3-methylphenyl)piperazine-1-carboxylate (I, 500 mg, 1.407 mmol) in 1,4-dioxane (5 mL) was added 3-(4-methoxybenzyl)dihydropyrimidine-2,4(1H,3H)-dione (330 mg, 1.407 mmol) followed by Cs₂CO₃ (917 mg, 2.81 mmol) and GPhos (40.7 mg, 0.070 mmol) at RT. The resulting reaction mixture was degassed for 10 min, and tris(dibenzylideneacetone)dipalladium(0) (129 mg, 0.141 mmol) was added. The reaction mixture was stirred at 110° C. for 16 h. It was then cooled to room temperature, filtered through a bed of celite, and concentrated under reduced pressure to get the crude product. This residue was purified by silica gel column chromatography eluted with 35% ethyl acetate in petroleum ether to afford pure tert-butyl 4-(4-(3-(4-methoxybenzyl)-2,4-dioxotetrahydropyrimidin-1(2H)-yl)-3-methylphenyl)piperazine-1-carboxylate (II, 200 mg, 25.6% yield) as a pale yellow liquid. [M+H−OtBu]P=453.2.

Step 2: Preparation of 1-(2-methyl-4-(piperazin-1-yl)phenyl)dihydropyrimidine-2,4(1H,3H)-dione (C-126). To a stirred solution of tert-butyl 4-(4-(3-(4-methoxybenzyl)-2,4-dioxotetrahydropyrimidin-1(2H)-yl)-3-methylphenyl)piperazine-1-carboxylate (II, 200 mg, 0.393 mmol) in TFA (2 mL) was added triflic acid (0.349 mL, 3.93 mmol) and stirred at 50° C. for 2 h. The reaction mixture was cooled to room temperature and concentrated under reduced pressure to get 1-(2-methyl-4-(piperazin-1-yl)phenyl)dihydropyrimidine-2,4(1H,3H)-dione, TFA (C-126, 205 mg, 98% yield) as dark brown solid. It was used for the next step without further purification. [M+H]⁺=289.2.

Table 68 summarizes the intermediates that were synthesized using a procedure similar to Example S81.

TABLE 68
Intermediate Compounds Prepared via a Procedure Similar
to Example S81.

| Intermediate No. | Structure |
|---|---|
| C-126 | 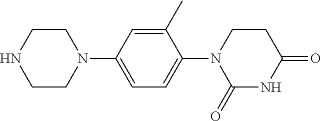 [SMILES: Cc1cc(N2CCNCC2)ccc1N1CCC(=O)NC1=O] |
| C-127 | 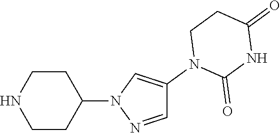 [SMILES: O=C1CCN(c2cnn(C3CCNCC3)c2)C(=O)N1] |

### Example S82. Synthesis of (R)-1-(4-(pyrrolidin-3-yloxy)phenyl)dihydropyrimidine-2,4(1H,3H)-dione (C-172)

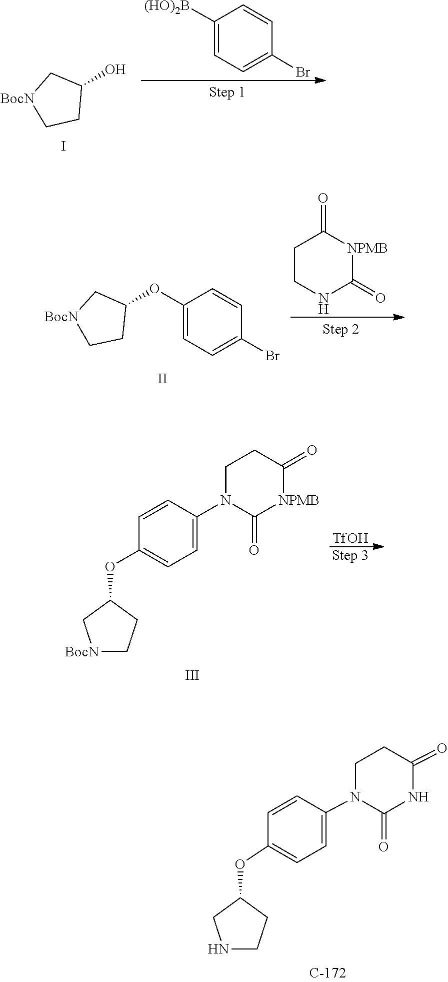

Step 1: Preparation of tert-butyl (R)-3-(4-bromophenoxy)pyrrolidine-1-carboxylate (II). To a solution of tert-butyl (R)-3-hydroxypyrrolidine-1-carboxylate (I, 2.0 g, mmol) and (4-bromophenyl)-boronic acid (3.22 g, 16.02 mmol) in DCM (50.0 mL) was added copper (II) acetate (3.88 g, 21.36 mmol) followed by pyridine (1.728 mL, 21.36 mmol) and DIPEA (3.73 mL, 21.36 mmol) at RT and the resulting solution was stirred at RT for 16 h. The reaction mixture was diluted with ice-cold water and extracted with DCM (3×30 mL). The combined organic layer was washed with brine, dried over sodium sulphate, and then concentrated under reduced pressure to give the crude product. The crude was purified by flash column chromatography using silica gel (100-200 mesh) with 15% ethyl acetate/pet ether to afford tert-butyl (R)-3-(4-bromophenoxy)pyrrolidine-1-carboxylate (II, 510 mg, 12.84% yield). [M+H−Boc]⁺=242.0.

Step 2: Preparation of tert-butyl (R)-3-(4-(3-(4-methoxybenzyl)-2,4-dioxotetrahydropyrimidin-1(2H)-yl)phenoxy)pyrrolidine-1-carboxylate (III). To a stirred solution of tert-butyl (R)-3-(4-bromophenoxy)pyrrolidine-1-carboxylate (II, 100 mg, 0.269 mmol) in 1,4-dioxane (2 mL) was added 3-(4-methoxybenzyl)dihydropyrimidine-2,4(1H,3H)-dione (63.0 mg, 0.269 mmol) and potassium phosphate, tribasic (140 mg, 0.806 mmol). The resulting mixture was purged with nitrogen gas for 10 min. Then copper(I) iodide (38.4 mg, mmol) was added followed by N,N′-dimethylethylenediamine (47.4 mg, 0.538 mmol) to the reaction mixture and stirred at 100° C. for 16 h. The reaction mixture was cooled to RT, filtered through celite bed, washed with ethyl acetate (3×10 mL), and concentrated under reduced pressure to get the crude product. The crude was purified by flash column chromatography on silica gel (100-200 mesh) with 40% ethyl acetate to get pure tert-butyl (R)-3-(4-(3-(4-methoxybenzyl)-2,4-dioxotetrahydropyrimidin-1(2H)-yl)phenoxy)pyrrolidine-1-carboxylate (III, 75 mg, 51.3% yield) as an off-white solid. [M+H-OtBu]⁺=440.0.

Step 3: Preparation of (R)-1-(4-(pyrrolidin-3-yloxy)phenyl)dihydropyrimidine-2,4(1H,3H)-dione (C-172). To a stirred solution of tert-butyl (R)-3-(4-(3-(4-methoxybenzyl)-2,4-dioxotetrahydropyrimidin-1(2H)-yl)phenoxy)pyrrolidine-1-carboxylate (III, 70 mg, 0.129 mmol) in TFA (0.7 mL) was added triflic acid (0.114 mL, 1.285 mmol) and stirred at 50° C. for 4 h. The volatiles were removed under reduced pressure to get the residue which was then dissolved in the acetonitrile (20 mL) and concentrated it to get brown residue. It was taken in the diethyl ether (30 mL), stirred it for 10 min, then filtered to get (R)-1-(4-(pyrrolidin-3-yloxy)phenyl)dihydropyrimidine-2,4(1H,3H)-dione, TFA (C-172, 90 mg) as white solid. It was used for the next step without further purification. [M+H]⁺=276.1.

Table 69 summarizes the intermediates that were synthesized using a procedure similar to Example S82.

TABLE 69
Intermediate Compounds Prepared via a Procedure Similar
to Example S82.

| Intermediate | |
|---|---|
| No. | Structure |
| C-172 | 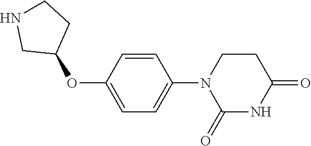 [SMILES: O=C1CCN(c2ccc(O[C@@H]3CCNC3)cc2)C(=O)N1] |
| C-174 | 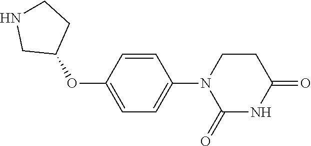 [SMILES: O=C1CCN(c2ccc(O[C@H]3CCNC3)cc2)C(=O)N1] |

### Example S83. Synthesis of 1-(4-(azetidin-3-yl)phenyl)dihydropyrimidine-2,4(1H,3H)-dione (C-179)

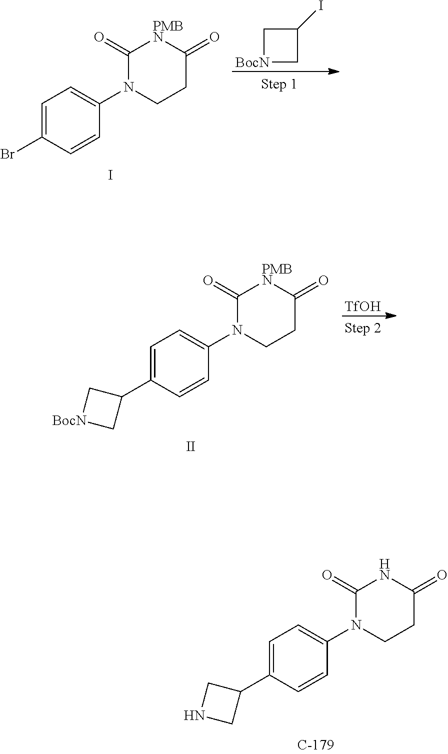

Step 1: Preparation of of tert-butyl 3-(4-(3-(4-methoxybenzyl)-2,4-dioxotetrahy dropyrimidin-1(2H)-yl)phenyl)azetidine-1-carboxylate (II). To a stirred solution of 1-(4-bromophenyl)-3-(4-methoxybenzyl)dihydropyrimidine-2,4(1H,3H)-dione (I, 1.209 g, 2.83 mmol) and tert-butyl 3-iodoazetidine-1-carboxylate (800 mg, 2.83 mmol) in 1,4-dioxane (8 mL) was added tributylamine (1.571 g, 8.48 mmol). The resulting reaction mixture was purged with nitrogen for 10 min. 4-CzIPN (178 mg, 0.226 mmol) and NiBr₂·dtbpy (553 mg, 1.130 mmol) were added into the reaction mixture and stirred at room temperature for 48 h under the irradiation of blue LED. The reaction mixture was filtered through a celite bed and washed with ethyl acetate (30 mL). The filtrate was concentrated under reduced pressure to obtain the crude compound. The crude compound was purified by column chromatography using silica gel (230-400 mesh) with 40% ethyl acetate/pet ether to obtain tert-butyl 3-(4-(3-(4-methoxybenzyl)-2,4-dioxotetrahydropyrimidin-1(2H)-yl)phenyl)azetidine-1-carboxylate (II, 240 mg, 17% yield) as a yellow gummy liquid. [M+H-Boc]⁺=366.2.

Step 2: Preparation of 1-(4-(azetidin-3-yl)phenyl)dihydropyrimidine-2,4(1H,3H)-dione, 2 TFA (C-179). To a stirred solution of tert-butyl 3-(4-(3-(4-methoxybenzyl)-2,4-dioxotetrahydropyrimidin-1(2H)-yl)phenyl)azetidine-1-carboxylate (II, 260 mg, 0.536 mmol) in TFA (3 mL) was added triflic acid (0.476 mL). The resulting reaction mixture was stirred at 50° C. for 3 h. The reaction mixture was concentrated under reduced pressure to obtain the crude compound. The crude compound was co-distilled with acetonitrile (2×5 mL), triturated with diethyl ether (2×5 mL) and dried under reduced pressure to obtain 1-(4-(azetidin-3-yl)phenyl)dihydropyrimidine-2,4(1H,3H)-dione, 2 TFA (C-179, 170 mg, 64% yield) as a yellow solid. It was used for the next step without further purification. [M+H]⁺=246.1.

Table 70 summarizes the intermediates that were synthesized using a procedure similar to Example S83.

TABLE 70
Intermediate Compounds Prepared via a Procedure Similar
to Example S83.

| Intermediate | |
|---|---|
| No. | Structure |
| C-178 | 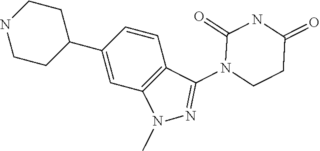 [SMILES: Cn1nc(N2CCC(=O)NC2=O)c2ccc(C3CCNCC3)cc21] |
| C-179 | 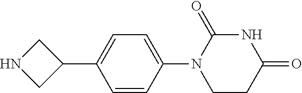 [SMILES: O=C1CCN(c2ccc(C3CNC3)cc2)C(=O)N1] |

### Example S84. Synthesis of 3-(4-(piperidin-4-yl)-1H-1,2,3-triazol-1-yl)piperidine-2,6-dione (C-117)

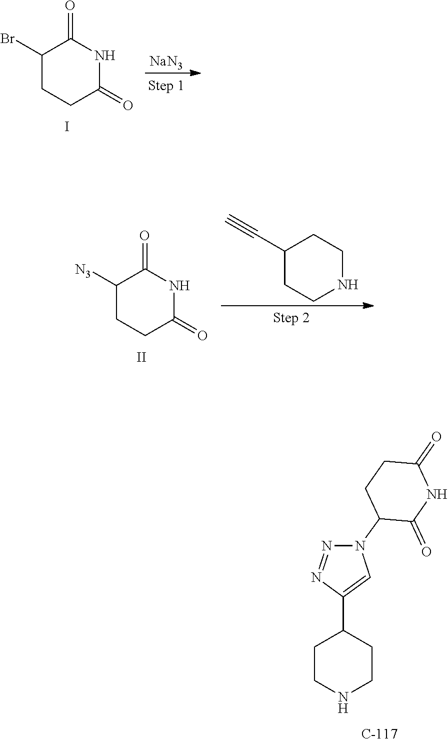

Step 1: Preparation of 3-azidopiperidine-2,6-dione (II).To 20 mL vial was added sodium azide (305 mg, 4.69 mmol), 3-bromopiperidine-2,6-dione (I, 180 mg, 0.937 mmol), water (1 mL), and acetone (2 mL). This reaction mixture was stirred at room temperature overnight. The volatiles were then removed under a nitrogen stream then the mixture was diluted into water (25 mL) and ethyl acetate (25 mL). The aqueous layer was extracted with ethyl acetate thrice. The combined organic layers were washed with water then dried with brine and magnesium sulfate before filtration and concentration in vacuo. This organic extract furnished 3-azidopiperidine-2,6-dione (II, 74 mg, 51% yield). Used without further purification. [M−H] P=153.1.

Step 2: Preparation of 3-(4-(piperidin-4-yl)-1H-1,2,3-triazol-1-yl)piperidine-2,6-dione (C-117). To a vial containing 3-azidopiperidine-2,6-dione (II, 74 mg, 0.487 mmol) was added copper(II) sulfate (35.3 mg, 0.221 mmol, L-Ascorbic acid sodium salt (43.8 mg, 0.221 mmol), 4-ethynylpiperidine (48.3 mg, 0.442 mmol, water (4.00 mL), and THF (4 mL). The vial was then sealed and the reaction mixture was stirred at room temperature for 72 h over which time a green color evolved. The crude material was purified via preparative SFC chromatography with the following conditions: Column: Waters 2-EP, 19 mm×250 mm, 5 μm particles; Flow Rate: 75 mL/min; Column Temperature: 40° C. Fraction collection was triggered by UV (254). Fractions containing the desired product were combined and dried via centrifugal evaporation. This afforded 3-(4-(piperidin-4-yl)-1H-1,2,3-triazol-1-yl)piperidine-2,6-dione as a white solid (C-117, 29 mg, 25% yield). [M+H]⁺=264.5.

### Example S85. Synthesis of 1-(2-fluoro-4-(piperazin-1-yl)phenyl)dihydropyrimidine-2.4(1H,3H)-dione (C-1241

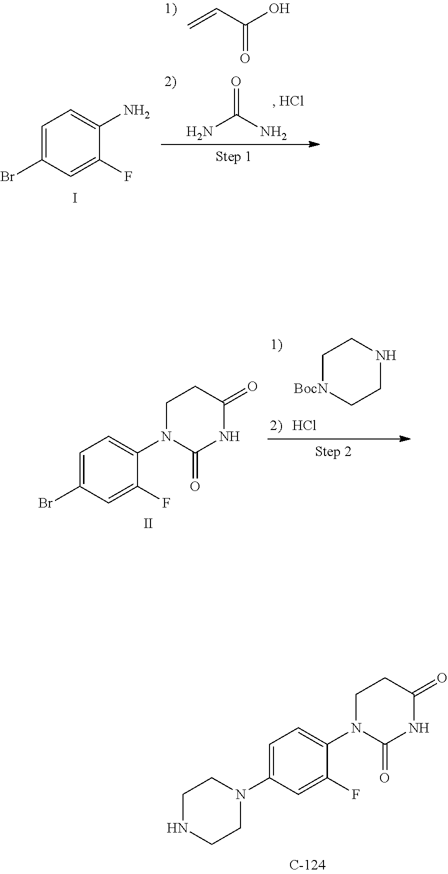

Step 1: Preparation of 1-(4-bromo-2-fluorophenyl)dihydropyrimidine-2,4(1H,3H)-dione (II). A 20 mL vial was charged with 4-bromo-2-fluoroaniline (I, 500 mg, 2.63 mmol), toluene (7 mL), and acrylic acid (0.217 mL, 3.16 mmol). The vial was then sealed and placed under an inert atmosphere before being heated to 90° C. and stirred for 3 h. Reaction progress was monitored by TLC. Upon apparent consumption of aniline starting material, the crude reaction mix was charged with urea (190 mg, 3.16 mmol), and hydrochloric acid in dioxane solution (0.658 mL, 4 M, 3.16 mmol). This mixture was then resealed and continued to stir at 90° C. for 3 d. The reaction was then quenched with the addition of saturated sodium bicarbonate solution. The aqueous mixture was then extracted 3× with methylene chloride. The combined organic layers were then dried with brine and magnesium sulfate before filtration. This organic extract was coated onto silica gel under reduced pressure and purified in a gradient elution system of 0>35% ethyl acetate in heptanes to afford 1-(4-bromo-2-fluorophenyl)dihydropyrimidine-2,4(1H,3H)-dione as a light orange crystalline solid (XVII, 150 mg, 20% yield). [M+H]⁺=287.2.

Step 2: Preparation of 1-(2-fluoro-4-(piperazin-1-yl)phenyl)dihydropyrimidine-2,4(1H,3H)-dione (C-124). To a microwave vial was added 1-(4-bromo-2-fluorophenyl)dihydropyrimidine-2,4(1H,3H)-dione (II, 50.0 mg, 0.174 mmol), sodium tert-butoxide (33.5 mg, 0.348 mmol), 1-boc-piperazine (35.7 mg, 0.192 mmol), XPhos Pd G4 (15.0 mg, 0.017 mmol), and toluene (3.5 mL). The vial was then sealed with a rubber septum and the atmosphere was evacuated and purged with dinitrogen 3×. The solution was then sparged with dinitrogen while sonicated, then sealed. This sealed vial was then irradiated in a microwave at 160° C. for 10 min causing the evolution of a precipitant. These solids were then isolated via filtration and washed with fresh toluene before being suspended in a solution of hydrochloric acid in dioxane (4 mL, 1 M, 4.0 mmol) and stirred at 70° C. for 1 h. Volatiles and additional acid were removed under nitrogen stream to yield 1-(2-fluoro-4-(piperazin-1-yl)phenyl)dihydropyrimidine-2,4(1H,3H)-dione as an amorphous solid (C-124, 25 mg, 49% yield). [M+H]⁺=293.3.

### Example S86. Synthesis of 1-(4-(4-hydroxypiperidin-4-yl)phenyl)dihydropyrimidine-2,4(1H,3H)-dione (C-129)

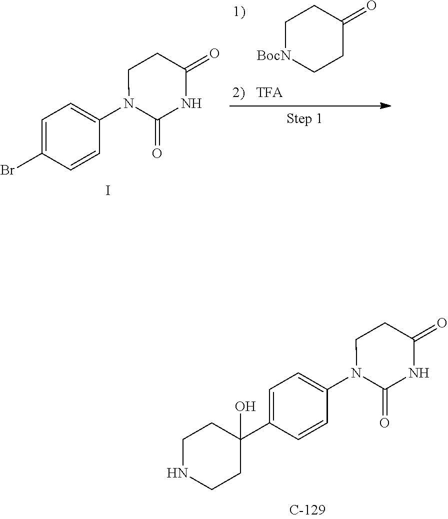

Step 1: Preparation of 1-(4-(4-hydroxypiperidin-4-yl)phenyl)dihydropyrimidine-2,4(1H,3H)-dione (C-129). To a test tube was added 1-(4-bromophenyl)dihydropyrimidine-2,4(1H,3H)-dione (I, 210 mg, 0.780 mmol), and 2 mL THF. This test tube was then sealed and placed under nitrogen atmosphere before being cooled to −78° C. n-Butyl lithium in hexanes (0.975 mL, 1.6 M, 1.560 mmol) was then added dropwise under inert atmosphere. The solution was stirred at −78° C. for 30 mins, then tert-butyl 4-oxopiperidine-1-carboxylate (120 mg, 0.6 mmol) in 1 mL THF was added dropwise over 10 mins with strong stirring. This reaction mixture was then stirred at −78° C. for 2 hours before being removed from the ice bath and allowed to warm to RT and stir overnight. Reaction mix was then quenched with saturated ammonium chloride solution and purified on reverse phase preparatory HPLC in 5>95% acetonitrile in water with 0.1% trifluoroacetic acid modifier. Relevant fractions were combined and concentrated under reduced pressure, then suspended in 20% v/v trifluoroacetic acid in methylene chloride and stirred for 2 h at RT. Volatiles were removed under nitrogen stream, yielding 1-(4-(4-hydroxypiperidin-4-yl)phenyl)dihydropyrimidine-2,4(1H,3H)-dione as a dark oil (C-129, 103 mg, 59% yield). [M+H]⁺=290.6.

### Example S86a. Synthesis of 1-(3-methyl-4-(piperazin-1-yl)phenyl)dihydropyrimidine-2,4(1H,3H)-dione (C-137)

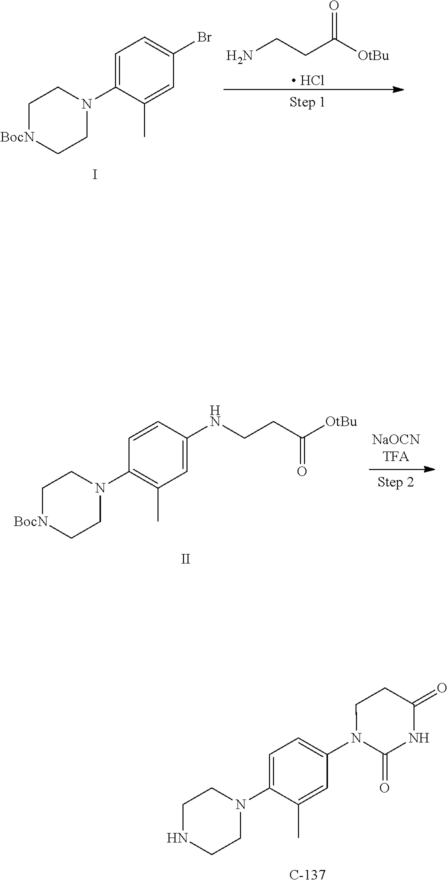

Step 1: Preparation of tert-butyl 4-(4-((3-(tert-butoxy)-3-oxopropyl)amino)-2-methylphenyl)piperazine-1-carboxylate (II). To a microwave vial was added tert-butyl 4-(4-bromo-2-methylphenyl)piperazine-1-carboxylate (I, 107 mg, 0.3 mmol), sodium tert-butoxide (86.0 mg, 0.9 mmol), tert-butyl 3-aminopropanoate HCl salt (54.5 mg, 0.3 mmol), XPhos Pd G4 (12.9 mg, 0.015 mmol), and toluene (1.5 mL). The vial was then sealed with a rubber septum and the atmosphere was evacuated and purged with dinitrogen 3×. The solution was then sparged with dinitrogen while sonicated, then quickly sealed. This sealed vial was then irradiated in a microwave at 160° C. for 15 min. The crude reaction mix was then purified via reverse phase preparatory HPLC in 5>95% acetonitrile in water with 0.1% trifluoroacetic acid modifier. Relevant fractions were combined and concentrated under reduced pressure to afford tert-butyl 4-(4-((3-(tert-butoxy)-3-oxopropyl)amino)-2-methylphenyl)piperazine-1-carboxylate as an off-white solid (II, 58 mg, 46% yield). [M+H]⁺=420.9.

Step 2: Preparation of 1-(3-methyl-4-(piperazin-1-yl)phenyl)dihydropyrimidine-2,4(1H,3H)-dione (C-137) Vial charged with tert-butyl 4-(4-((3-(tert-butoxy)-3-oxopropyl)amino)-2-methylphenyl)piperazine-1-carboxylate (II, 58 mg, 0.138 mmol), sodium cyanate (44.9 mg, 0.691 mmol), 1,4-dioxane (2.0 mL), and trifluoroacetic acid (0.5 mL). Reaction mixture was placed under nitrogen atmosphere and stirred at 70° C. for 3 d. After this time, the reaction was removed from heat and allowed to cool to RT. The crude mixture was then purified via reverse phase preparatory HPLC in 5>95% acetonitrile in water with 0.1% trifluoroacetic acid modifier. Concentration of relevant fractions under reduced pressure furnished 1-(3-methyl-4-(piperazin-1-yl)phenyl)dihydropyrimidine-2,4(1H,3H)-dione as an amorphous solid (C-137, 25 mg, 62% yield). [M+H]⁺=289.1.

### Example S87. Synthesis of 1-(1-(piperidin-4-yl)-1H-indazol-3-yl)dihydropyrimidine-2,4(1H,3H)-dione (C-140)

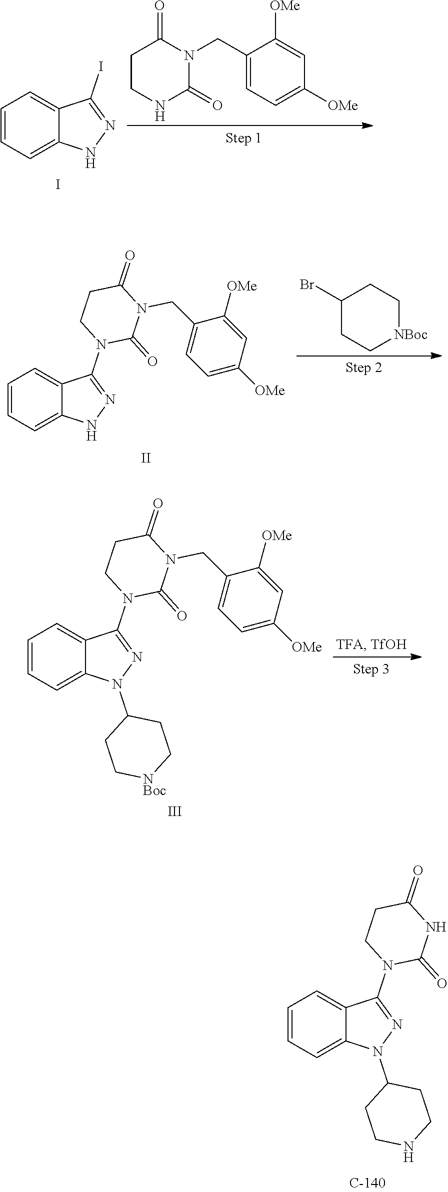

Step 1: Preparation of 3-(2,4-dimethoxybenzyl)-1-(1H-indazol-3-yl)dihydropyrimidine-2,4(1H,3H)-dione (II). A vial was charged with copper(I) iodide (143 mg, 0.750 mmol), tripotassium phosphate (318 mg, 1.500 mmol), 3-iodo-1H-indazole (I, 122 mg, 0.5 mmol), 3-(2,4-dimethoxybenzyl)dihydropyrimidine-2,4(1H,3H)-dione (132 mg, 0.5 mmol), and dioxane (2.5 mL). The vial was sealed and the atmosphere was evacuated and purged with nitrogen 3×. Reaction mixture was then sparged with nitrogen while stirring at room temperature for 10 mins. After this time, (1S,2S)—N1,N2-dimethylcyclohexane-1,2-diamine (79 0.500 mmol) added in one portion and sparging was continued until green color evolved. The vent needle was then removed and the reaction mixture was heated to 100° C. with overnight stirring under positive nitrogen pressure. The crude reaction was removed from the heat and allowed to cool to room temperature before filtration. The filtrate was then purified via reverse phase preparatory HPLC in 5>95% acetonitrile in water with 0.1% trifluoroacetic acid modifier to yield 3-(2,4-dimethoxybenzyl)-1-(1H-indazol-3-yl)dihydropyrimidine-2,4(1H,3H)-dione as an off white solid (II, 67 mg, 35% yield). [M+H]⁺=381.3.

Step 2: Preparation of tert-butyl 4-(3-(3-(2,4-dimethoxybenzyl)-2,4-dioxotetrahydropyrimidin-1(2H)-yl)-1H-indazol-1-yl)piperidine-1-carboxylate (III). To the vial containing 3-(2,4-dimethoxybenzyl)-1-(1H-indazol-3-yl)dihydropyrimidine-2,4(1H,3H)-dione (II, 66 mg, 0.173 mmol) was added Ir[dFFppy]₂-(4,4′-dCF₃bpy)PF₆ (1.451 mg, 1.388 μmol), and acetonitrile (1.735 mL). Mix was stirred for 5 min at room temperature. Lithium tert-butoxide (520 0.520 mmol) and water (31.3 IA, 1.735 mmol) were added followed by 60 seconds of sonication under open atmosphere. Copper bis(2,2,6,6-tetramethyl-3,5-heptanedionate) (14.92 mg, 0.035 mmol) was added and the mixture was stirred uncapped for 3 minutes. Tris(trimethylsilyl)silanol (134 μl, 0.434 mmol) and tert-butyl 4-bromopiperidine-1-carboxylate (115 mg, 0.434 mmol) were added and the vessel was capped and vented with an 18G needle. Stirred at room temperature for 4 h while irradiated with 440 nm blue LEDs. After this time, LCMS confirmed product generation and vial was removed from photoreactor and diluted with ethyl acetate. KF on alumina (300 mg) and tetrabutylammonium chloride (300 mg) were added to quench and the suspension was stirred at room temperature overnight. Solids were removed via filtration and the resulting eluent was then concentrated under a nitrogen stream before suspension in DMSO. This solution was purified via reverse phase preparatory HPLC in 5>95% acetonitrile in water with 0.1% trifluoroacetic acid modifier then concentrated under reduce pressure to afford tert-butyl 4-(3-(3-(2,4-dimethoxybenzyl)-2,4-dioxotetrahydropyrimidin-1(2H)-yl)-1H-indazol-1-yl)piperidine-1-carboxylate as an off white crystalline solid (III, 37 mg, 38% yield). [M+H]⁺=564.5.

Step 3: Preparation of 1-(1-(piperidin-4-yl)-1H-indazol-3-yl)dihydropyrimidine-2,4(1H,3H)-dione (C-140) tert-butyl-4-(3-(3-(2,4-dimethoxybenzyl)-2,4-dioxotetrahydropyrimidin-1(2H)-yl)-1H-indazol-1-yl)piperidine-1-carboxylate (III, 37 mg, 0.064 mmol) in a 20 mL vial was suspended in trifluoroacetic acid (0.3 mL) and triflic acid (0.3 mL) and stirred overnight at room temperature. Volatiles were removed under nitrogen stream and residual acid removed via azeotroping with methanol 3×. Concentration in vacuo yielded 1-(1-(piperidin-4-yl)-1H-indazol-3-yl)dihydropyrimidine-2,4(1H,3H)-dione (C-140, 20 mg, quantitative yield). [M+H]⁺=314.3.

### Example S88. Synthesis of 1-(1-methyl-6-(piperazin-1-yl)-1H-indazol-3-yl)dihydropyrimidine-2,4(1H,3H)-dione (C-148)

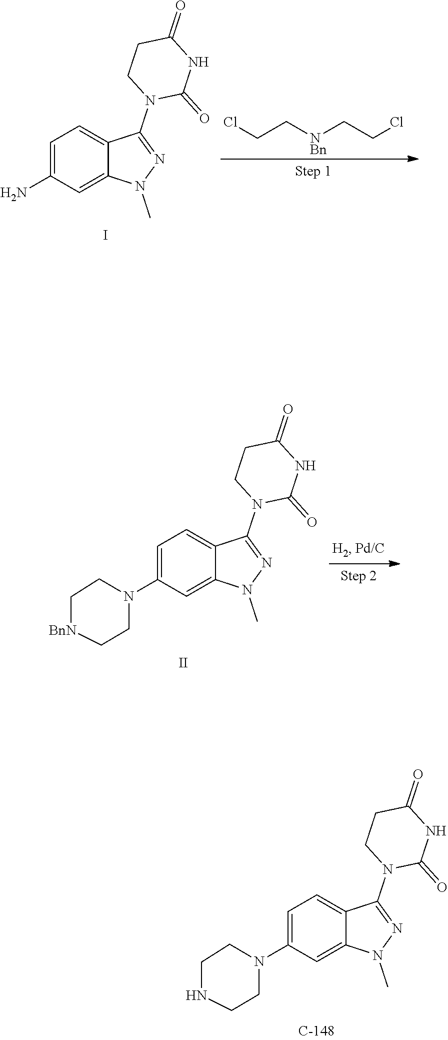

Step 1: Preparation of 1-(6-(4-benzylpiperazin-1-yl)-1-methyl-1H-indazol-3-yl)dihydropyrimidine-2,4(1H,3H)-dione (II). A 20 mL vial was charged with 1-(6-amino-1-methyl-1H-indazol-3-yl)dihydropyrimidine-2,4(1H,3H)-dione (I, 51.9 mg, 0.2 mmol), acetonitrile (1 mL), and triethylamine (139 μL, 1.000 mmol). Reaction mix was stirred for 15 mins at room temperature. N-benzyl-2-chloro-N-(2-chloroethyl)ethan-1-amine (81 μL, 0.400 mmol) was then added. Crude reaction mix was then heated to 80 C and stirred for 2 days. The vessel was then removed from heat and allowed to cool to room temperature before being purified on reverse phase preparatory HPLC in 5>95% acetonitrile in water with 0.1% trifluoroacetic acid modifier. Relevant fractions were combined and concentrated under reduced pressure to afford 1-(6-(4-benzylpiperazin-1-yl)-1-methyl-1H-indazol-3-yl)dihydropyrimidine-2,4(1H,3H)-dione as an off white solid (II, 67 mg, 80% yield). [M+H]⁺=419.4.

Step 2: Preparation of 1-(1-methyl-6-(piperazin-1-yl)-1H-indazol-3-yl)dihydropyrimidine-2,4(1H,3H)-dione (C-148) To a vial containing 1-(6-(4-benzylpiperazin-1-yl)-1-methyl-1H-indazol-3-yl)dihydropyrimidine-2,4(1H,3H)-dione (II, 67 mg, 0.160 mmol) was charged with ethanol (2 mL) and palladium on carbon (30% w/w, 11.4 mg, 0.32 mmol). The atmosphere was then evacuated and purged with nitrogen 3×. A balloon filled with hydrogen gas was then affixed to the vessel and the reaction was stirred at room temperature for 72 h. The balloon was then removed and the reaction mix filtered over celite. Concentration under reduced pressure afforded 1-(1-methyl-6-(piperazin-1-yl)-1H-indazol-3-yl)dihydropyrimidine-2,4(1H,3H)-dione (C-148, 53 mg, quantitative yield). [M+H]⁺=329.3.

### Example S89. Synthesis of 1-(4-(3-fluoropiperidin-4-yl)phenyl)dihydropyrimidine-2,4(1H,3H)-dione (C-155a and C-155b)

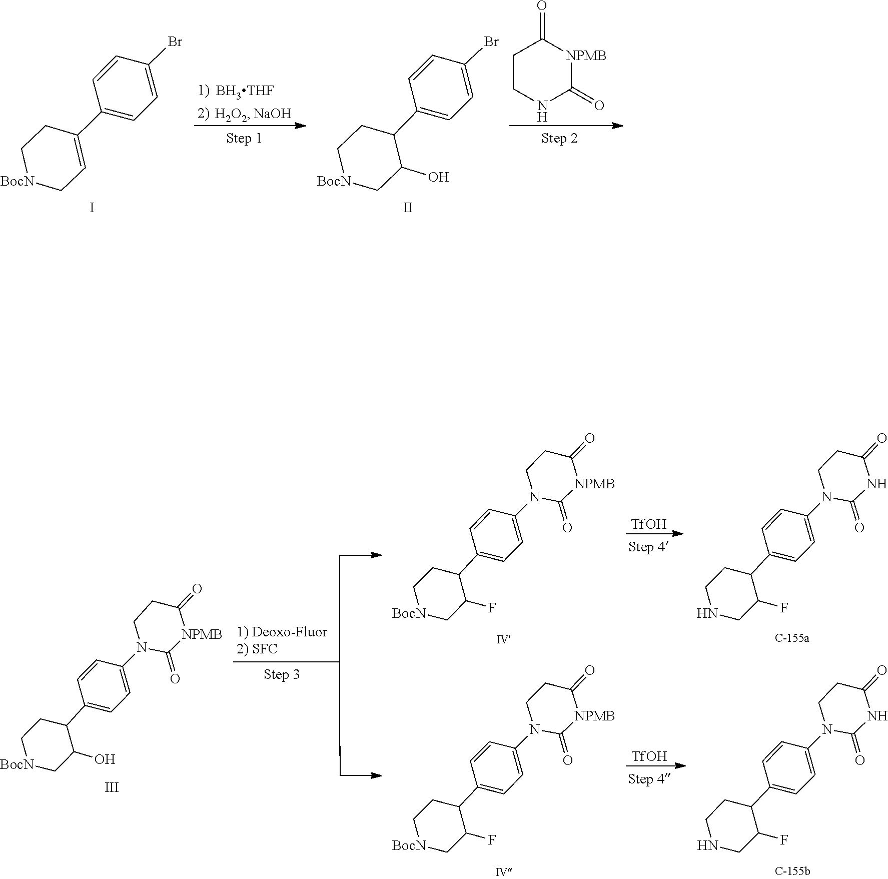

Step 1: Preparation of tert-butyl 4-(4-bromophenyl)-3-hydroxypiperidine-1-carboxylate (II). To a cooled stirred solution of tert-butyl 4-(4-bromophenyl)-3,6-dihydropyridine-1(2H)-carboxylate (I, 2.0 g, 5.91 mmol) in THF (15.0 mL) was added BH₃·THF (8.87 mL, 8.87 mmol) at 0° C. dropwise. The resulting reaction was slowly warmed to room temperature and stirred for 3 h. The reaction mixture was cooled to 0° C. and treated with 10% sodium hydroxide (10 ml) dropwise, followed by hydrogen peroxide solution (35%, 2.59 mL, 29.6 mmol). The reaction mixture was slowly warmed to room temperature and stirred for 5 h. The reaction mixture was treated with ice-cold water and extracted with EtOAc (3×40 mL). The combined organics were dried over Na₂SO₄ and concentrated under reduced pressure to obtain the crude compound tert-butyl 4-(4-bromophenyl)-3-hydroxypiperidine-1-carboxylate (II, 750 mg, 34.5% yield) as an off-white solid. It was used for the next step without further purification. [M+H-Boc]⁺=256.0.

Step 2: Preparation of tert-butyl 3-hydroxy-4-(4-(3-(4-methoxybenzyl)-2,4-dioxotetrahydropyrimidin-1(2H)-yl)phenyl)piperidine-1-carboxylate (III). To a stirred solution of tert-butyl 4-(4-bromophenyl)-3-hydroxypiperidine-1-carboxylate (II, 1.0 g, 2.81 mmol) and 3-(4-methoxybenzyl)dihydropyrimidine-2,4(1H,3H)-dione (0.778 g, 3.09 mmol) in 1,4-dioxane (20 mL) was added potassium phosphate, tribasic (1.192 g, 5.61 mmol) followed by copper(I) iodide (0.535 g, 2.81 mmol) and N,N-dimethylethylene1,2-diamine (0.495 g, 5.61 mmol). Then, the reaction mixture was stirred at 100° C. for 16 h. The reaction mixture was cooled to room temperature, diluted with water, and extracted with EtOAc (3×100 mL). The combined organics were dried over Na₂SO₄ and concentrated under reduced pressure. The crude residue was purified by column chromatography using silica gel (230-400 mesh) with 80% ethyl acetate in petroleum ether to obtain tert-butyl 3-hydroxy-4-(4-(3-(4-methoxybenzyl)-2,4-dioxotetrahydropyrimidin-1(2H)-yl)phenyl)piperidine-1-carboxylate (III, 1.5 g, 66.1% yield) as a white solid. [M+H-Boc]⁺=410.2.

Step 3: Preparation of tert-butyl 3-fluoro-4-(4-(3-(4-methoxybenzyl)-2,4-dioxotetrahydropyrimidin-1(2H)-yl)phenyl)piperidine-1-carboxylate (IV) & Chiral Separation. To a stirred solution of tert-butyl 3-hydroxy-4-(4-(3-(4-methoxybenzyl)-2,4-dioxotetrahydropyrimidin-1(2H)-yl)phenyl)piperidine-1-carboxylate (III, 1.0 g, 1.236 mmol) in acetonitrile (20.0 mL) was added deoxofluor 50% in toluene (1.368 mL, 3.71 mmol) at 0° C. The reaction mixture was stirred at 0° C. for 30 min, slowly warmed to room temperature, and stirred for 2 h. The reaction mixture was quenched with saturated NH₄C₁ solution and extracted with EtOAc (3×100 mL). The combined organics were dried over Na₂SO₄ and concentrated under reduced pressure to obtain the crude compound. The crude compound was purified by column chromatography using silica gel (230-400 mesh) with 15% ethyl acetate in petroleum ether to obtain tert-butyl 3-fluoro-4-(4-(3-(4-methoxybenzyl)-2,4-dioxotetrahydropyrimidin-1(2H)-yl)phenyl)piperidine-1-carboxylate (IV, 600 mg, 87% yield) as white solid. The racemic compound was purified by chiral SFC to obtain tert-butyl 3-fluoro-4-(4-(3-(4-methoxybenzyl)-2,4-dioxotetrahydropyrimidin-1(2H)-yl)phenyl)piperidine-1-carboxylate (Peak-1) IV′ (210 mg) and tert-butyl 3-fluoro-4-(4-(3-(4-methoxybenzyl)-2,4-dioxotetrahydropyrimidin-1(2H)-yl)phenyl)piperidine-1-carboxylate (Peak-2) IV″ (210 mg). [M+H-OtBu]⁺=456.2.

SFC Method. Cellulose-4, 250×4.6 mm, 5.0 Flow: 3.0 mL/min. Co-Solvent: 25.0% IPA.

IV′ (Peak-1): RT 7.33 min; ee 99.7%.

IV″ (Peak-2): RT 8.48 min; ee 94.3%.

Step 4′: Preparation of 1-(4-(3-fluoropiperidin-4-yl)phenyl)dihydropyrimidine-2,4(1H,3H)-dione (Peak-1, C-155a). To a stirred solution of tert-butyl 3-fluoro-4-(4-(3-(4-methoxybenzyl)-2,4-dioxotetrahydropyrimidin-1(2H)-yl)phenyl)piperidine-1-carboxylate (IV′, Peak-1, 150 mg, 0.293 mmol) in TFA (4 mL) at room temperature was added triflic acid (0.260 mL, 2.93 mmol). The resulting reaction mixture was stirred at 50° C. for 2 h. The reaction mixture was concentrated under reduced pressure to obtain 1-(4-(3-fluoropiperidin-4-yl)phenyl)dihydropyrimidine-2,4(1H,3H)-dione, 2 TFA (C-155a, 120 mg) as a light brown solid. It was used for the next step without further purification. [M+H]⁺=292.2.

Step 4″: Preparation of 1-(4-(3-fluoropiperidin-4-yl)phenyl)dihydropyrimidine-2,4(1H,3H)-dione (Peak-2, C-155b). To a stirred solution of tert-butyl 3-fluoro-4-(4-(3-(4-methoxybenzyl)-2,4-dioxotetrahydropyrimidin-1(2H)-yl)phenyl)piperidine-1-carboxylate (IV″, Peak-2, 150 mg, 0.293 mmol) in TFA (4 mL) at room temperature was added triflic acid (0.260 mL, 2.93 mmol). The resulting reaction mixture was stirred at 50° C. for 2 h. The reaction mixture was concentrated under reduced pressure to obtain 1-(4-(3-fluoropiperidin-4-yl)phenyl)dihydropyrimidine-2,4(1H,3H)-dione, 2 TFA (C-155b, 120 mg) as a light brown solid. It was used for the next step without further purification. [M+H]⁺=292.1.

### Example S90. Synthesis of 1-(6-(piperazin-1-yl)pyridazin-3-yl)dihydropyrimidine-2,4(1H,3H)-dione (C-161)

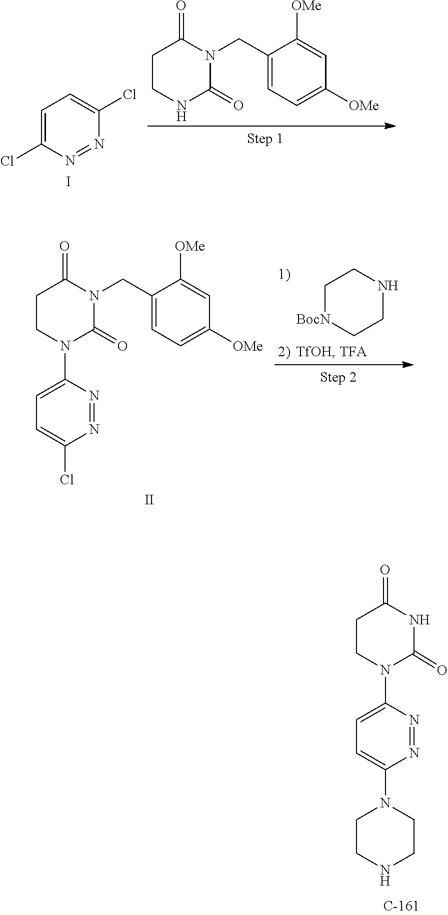

Step 1: Preparation of 1-(6-chloropyridazin-3-yl)-3-(2,4-dimethoxybenzyl)dihydropyrimidine-2,4(1H,3H)-dione (II). A microwave vial was charged with potassium carbonate (276 mg, 2.00 mmol), 3-(2,4-dimethoxybenzyl)dihydropyrimidine-2,4(1H,3H)-dione (264 mg, 1.00 mmol), 3,6-dichloropyridazine (I, 149 mg, 1.00 mmol), and DMF (4 mL). The vial was sealed and irradiated in the microwave at 150° C. for 4 h. The crude mixture was purified via reverse phase preparatory HPLC in 5>95% acetonitrile in water with trifluoroacetic acid modifier then concentrated under reduce pressure to afford 1-(6-chloropyridazin-3-yl)-3-(2,4-dimethoxybenzyl)dihydropyrimidine-2,4(1H,3H)-dione as an off-white solid (II, 53 mg, 14% yield). [M+H]⁺=377.3.

Step 2: Preparation of 1-(6-(piperazin-1-yl)pyridazin-3-yl)dihydropyrimidine-2,4(1H,3H)-dione (C-161). 1-(6-chloropyridazin-3-yl)-3-(2,4-dimethoxybenzyl)dihydropyrimidine-2,4(1H,3H)-dione (II, 53 mg, 0.141 mmol) was transferred to a microwave vial followed by 1-butanol (1 mL) and 1-boc-piperazine (28.8 mg, mmol). The vial was then sealed and irradiated at 150° C. for 8 h. This crude mixture was then concentrated under a nitrogen stream then purified on reverse phase preparatory HPLC in acetonitrile in water with 0.1% trifluoroacetic acid modifier. Relevant fractions were combined and concentrated under reduced pressure. This residue was then suspended in 0.3 mL trifluoroacetic acid and 0.3 mL triflic acid then stirred at room temperature for 3 h. Volatiles were removed under nitrogen stream and additional acid was removed via 3 rounds of methanol azeotrope under nitrogen stream. This afforded 1-(6-(piperazin-1-yl)pyridazin-3-yl)dihydropyrimidine-2,4(1H,3H)-dione (C-161, 23 mg, 59% yield). [M+H]⁺=277.3.

### Example S91. Synthesis of C-156a and C-156b

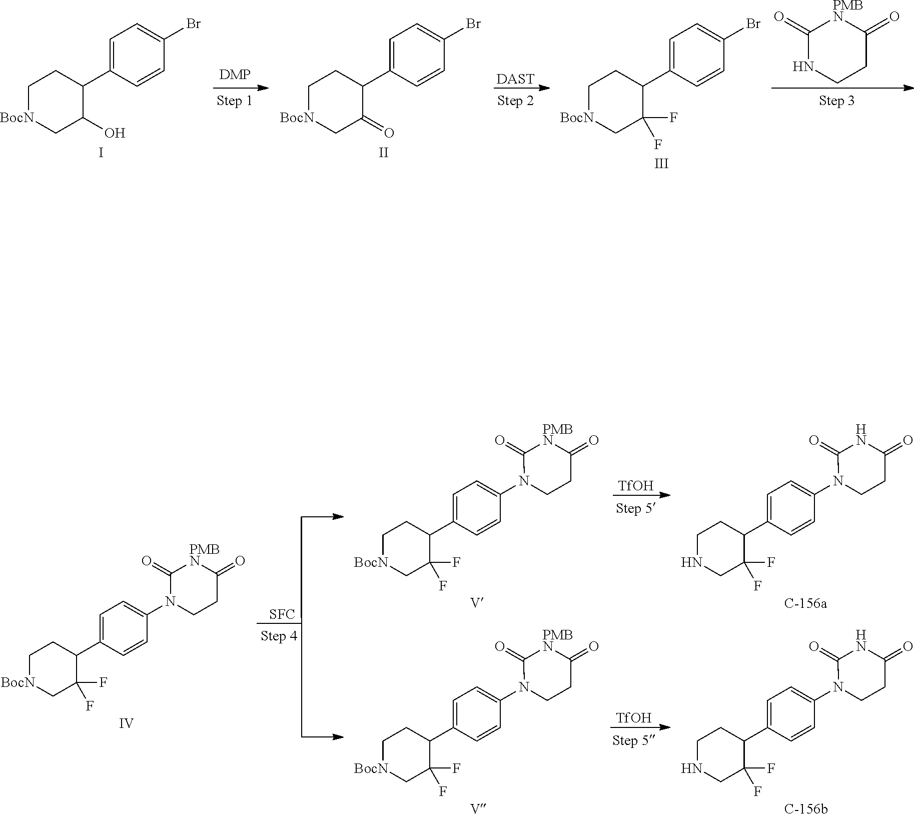

Step 1: Preparation of tert-butyl 4-(4-bromophenyl)-3-oxopiperidine-1-carboxylate (II). To a solution of tert-butyl 4-(4-bromophenyl)-3-hydroxypiperidine-1-carboxylate (I) (1.2 g, 3.27 mmol) in DCM (10 mL) and water (0.071 mL, 3.92 mmol) at 0° C. was added DMP (2.77 g, 6.53 mmol). The reaction was stirred for 4 h at RT. The reaction mixture was quenched via the addition of 10% sodium bicarbonate solution (20 mL) slowly at 0° C. This aqueous mixture was then extracted with DCM (3×20 mL). The combined organics were dried over Na₂SO₄ and concentrated under reduced pressure to obtain the crude compound. The crude was purified by column chromatography using silica gel (230-400 mesh) with 20-30% ethyl acetate/petroleum ether to obtain tert-butyl 4-(4-bromophenyl)-3-oxopiperidine-1-carboxylate (II, 900 mg, 57.8% yield) as a pale yellow liquid. [M+H−OtBu]⁺=298.0.

Step 2: Preparation of tert-butyl 4-(4-bromophenyl)-3,3-difluoropiperidine-1-carboxylate (III). To a cooled stirred solution of tert-butyl 4-(4-bromophenyl)-3-oxopiperidine-1-carboxylate (II) (800 mg, 1.678 mmol) in DCM (12 mL) at 0° C. was added DAST (0.773 mL, 4.19 mmol). The resulting reaction was slowly warmed to RT and stirred for 2 h. The reaction mixture was treated with 10% sodium bicarbonate (20 mL) slowly and then extracted with EtOAc (3×20 mL). The combined organics were dried over Na₂SO₄ and concentrated under reduced pressure to obtain the crude compound. The crude was purified by column chromatography using silica gel (230-400 mesh) with 25-35% ethyl acetate/pet ether to obtain tert-butyl 4-(4-bromophenyl)-3,3-difluoropiperidine-1-carboxylate (III, 600 mg, 85% yield) as a pale yellow liquid. [M+H−Boc]⁺=276.0.

Step 3: Preparation of (tert-butyl 3,3-difluoro-4-(4-(3-(4-methoxybenzyl)-2,4-dioxotetrahydro pyrimidin-1(2H)-yl)phenyl)piperidine-1 carboxylate (IV). To a stirred solution of tert-butyl 4-(4-bromophenyl)-3,3-difluoropiperidine-1-carboxylate (III, 600 mg, 1.43 mmol) in 1,4-dioxane (10 mL) was added 3-(4-methoxybenzyl)dihydropyrimidine-2,4(1H,3H)-dione (336 mg, 1.433 mmol), DMEDA (126 mg, 1.43 mmol) and potassium phosphate tribasic (608 mg, 2.87 mmol) at RT. The resulting solution was degassed for 10 min and then was added copper (I) iodide (273 mg, 1.43 mmol) at RT. This resulting solution was stirred at 100° C. for 16 h. The reaction mixture was quenched with 10% sodium bicarbonate solution (20 mL) slowly at 0° C. and then extracted with EtOAc (3×20 mL). The combined organics were dried over Na₂SO₄ and concentrated under reduced pressure to obtain the crude compound. The crude was purified by column chromatography using silica gel (230-400 mesh) with 25-35% ethyl acetate/pet ether to obtain tert-butyl 3,3-difluoro-4-(4-(3-(4-methoxybenzyl)-2,4-dioxotetrahydropyrimidin-1(2H)yl)phenyl)piperidine-1-carboxylate (IV, 550 mg, 72.6% yield) as a pale yellow liquid. [M+H-OtBu]⁺=474.2.

Step 4: Chiral SFC separation of tert-butyl 3,3-difluoro-4-(4-(3-(4-methoxybenzyl)-2,4-dioxotetrahydropyrimidin-1(2H)-yl)phenyl)piperidine-1-carboxylate V′ (Peak-1) & V″ (Peak-2). SFC Method: Cellulose-SC, 250×4.6 mm, 5.0 Flow: 3.0 mL/min. Co-Solvent: 50.0% IPA.

V′ (Peak-1): 250 mg; RT 3.21 min; ee 100%.

V″ (Peak-2): 220 mg; RT 4.23 min; ee 99.8%.

Step 5′: Preparation of 1-(4-(3,3-difluoropiperidin-4-yl)phenyl)dihydropyrimidine-2,4(1H,3H)-dione (C-156a). To a stirred solution of tert-butyl 3,3-difluoro-4-(4-(3-(4-methoxybenzyl)-2,4-dioxotetrahydro pyrimidin-1(2H)-yl)phenyl)piperidine-1-carboxylate Peak-1 (V′, 230 mg, 0.434 mmol) in TFA (4.0 mL) was added triflic acid (0.386 mL, 4.34 mmol) at RT. The resulting reaction mixture was stirred at 50° C. for 4 h. The reaction mixture was concentrated under reduced pressure to obtain a crude product, and it was washed with diethyl ether (3×30 mL) to get 1-(4-(3,3-difluoropiperidin-4-yl)phenyl)dihydropyrimidine-2,4(1H,3H)-dione, TFA (C-156a, 150 mg) as a pale yellow solid. It was used for the next step without further purification. [M+H]⁺=310.0.

Step 5″: Preparation of 1-(4-(3,3-difluoropiperidin-4-yl)phenyl)dihydropyrimidine-2,4(1H,3H)-dione (C-156b). To a stirred solution of tert-butyl 3,3-difluoro-4-(4-(3-(4-methoxybenzyl)-2,4-dioxotetrahydro pyrimidin-1(2H)-yl)phenyl)piperidine-1-carboxylate Peak-2 (V″, 200 mg, 0.370 mmol) in TFA (3.0 mL) was added triflic acid (0.329 mL, 3.70 mmol) at RT. The resulting reaction mixture was stirred at 50° C. for 2 h. The reaction mixture was concentrated under reduced pressure to obtain a crude product, and it was washed with diethyl ether (2×15.0 mL) to get 1-(4-(3,3-difluoropiperidin-4-yl)phenyl)dihydropyrimidine-2,4(1H,3H)-dione, TFA (C-156b, 200 mg) as gray color solid. It was used for the next step without further purification. [M+H]⁺=309.9.

### Example S92. Preparation of 1-(4-(((R)-1-cyanoethyl)amino)-5-(4-((1R,4R)-4-(2-((R)-4-(5-(2,4-dioxotetrahydropyrimidin-1(2H)-yl)pyridin-2-yl)-3-ethylpiperazin-1-yl)ethyl)cyclohexyl)-1H-1,2,3-triazol-1-yl)pyridin-2-yl)-1H-pyrazolo[3,4-b]pyridine-5-carbonitrile(P-394)

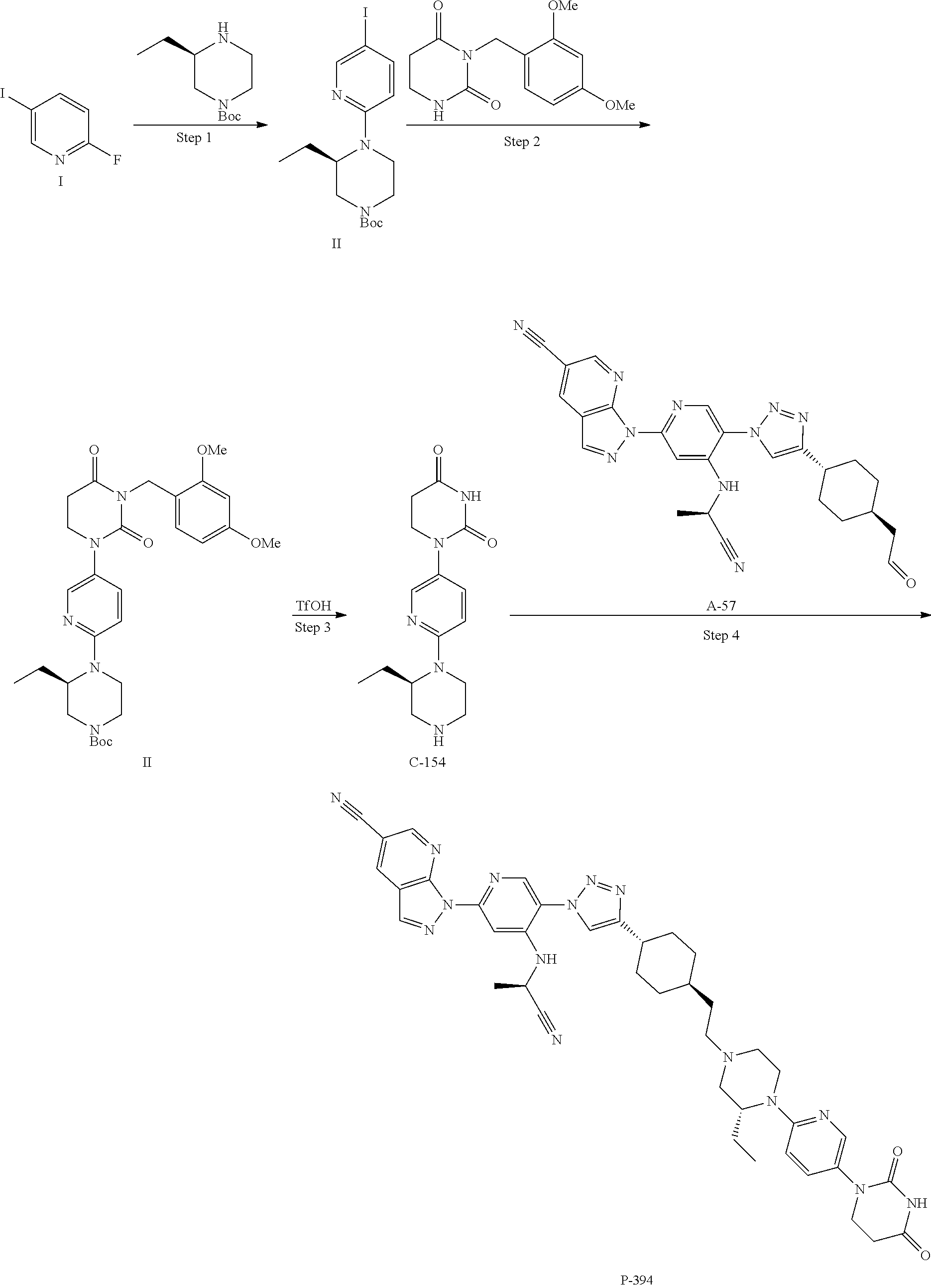

Step 1: Preparation of tert-butyl (R)-3-ethyl-4-(5-iodopyridin-2-yl)piperazine-1-carboxylate (II). A 20 mL vial equipped with stirbar was charged with 2-fluoro-5-iodopyridine (I, 200 mg, 0.897 mmol), tert-butyl (R)-3-ethylpiperazine-1-carboxylate (231 mg, 1.076 mmol), and Potassium Carbonate (248 mg, 1.794 mmol). The vial was then sealed and DMSO (897 μl) was added. The reaction vessel was then heated to 120° C. and stirred for six days. This reaction was quenched by the addition of water and EtOAc. The organic layer was then removed and the aqueuous layer was washed with EtOAc (3×). The combined organic layers dried over sodium sulfate, filtered and concentrated directly onto silica gel. The residue was then purified by normal phase silica gel chromatography (0% to 15% EtOAc in heptane), affording tert-butyl (R)-3-ethyl-4-(5-iodopyridin-2-yl)piperazine-1-carboxylate (II, 155 mg, 40.6% yield) as a colorless oil. [M+H]⁺=418.3.

Step 2: Preparation of tert-butyl (R)-4-(5-(3-(2,4-dimethoxybenzyl)-2,4-dioxotetrahydropyrimidin-1(2H)-yl)pyridin-2-yl)-3-ethylpiperazine-1-carboxylate (III). A 20 mL vial was charged with a stir bar, tert-butyl (R)-3-ethyl-4-(5-iodopyridin-2-yl)piperazine-1-carboxylate (II, 155 mg, 0.371 mmol), tribasic potassium phosphate (237 mg, 1.114 mmol), 3-(2,4-dimethoxybenzyl)dihydropyrimidine-2,4(1H,3H)-dione (98 mg, 0.371 mmol), and copper(I) iodide (106 mg, 0.557 mmol). The atmosphere was evacuated and purged with dinitrogen three times. 1,4-dioxane (3.7 mL) was then added, and the resulting solution was sparged wfor 15 mins while sonicated. (1S,2S)-cyclohexan-1,2-diamine (42.4 mg, 0.371 mmol) was then added and the vessel was quickly sealed. The reaction mixture was then placed in a preheated 90° C. block and stirred overnight. The crude reaction mix was then concentrated under dinitrogen, suspended in 2 m DMSO, and purified by reverse phase chromatography on a C-18 functionalized silica column using a gradient eluent of 5>95% acetonitrile in water (with TFA modifier). The resulting UV active fractions were analyzed via LCMS and the relevant fractions were combined and concentrated under reduced pressure to afford tert-butyl (R)-4-(5-(3-(2,4-dimethoxybenzyl)-2,4-dioxotetrahydropyrimidin-1(2H)-yl)pyridin-2-yl)-3-ethylpiperazine-1-carboxylate (III, 196 mg, 95.1% yield) and an off-white solid. [M+H]⁺=554.6.

Step 3: Preparation of (R)-1-(6-(2-ethylpiperazin-1-yl)pyridin-3-yl)dihydropyrimidine-2,4(1H,3H)-dione (C-154). To a 20 mL vial containing tert-butyl (R)-4-(5-(3-(2,4-dimethoxybenzyl)-2,4-dioxotetrahydropyrimidin-1(2H)-yl)pyridin-2-yl)-3-ethylpiperazine-1-carboxylate (iii, 196 mg) was added TFA (0.3 mL) and TfOH (0.3 mL) which caused an immediate color change to deep purple. The resulting mixture was stirred at room temperature for 3 h. Complete deprotection was observed via LCMS analysis. The mixture was concentrated under dinitrogen then residual acid was removed via three rounds of methanol azeotrope. The resulting solid was used as is. [M+H]⁺=304.4.

Step 4: Preparation of 1-(4-(((R)-1-cyanoethyl)amino)-5-(4-(trans-4-(2-((R)-4-(5-(2,4-dioxotetrahydropyrimidin-1(2H)-yl)pyridin-2-yl)-3-ethylpiperazin-1-yl)ethyl)cyclohexyl)-1H-1,2,3-triazol-1-yl)pyridin-2-yl)-1H-pyrazolo[3,4-b]pyridine-5-carbonitrile (P-394). A vial containing (R)-1-(6-(2-ethylpiperazin-1-yl)pyridin-3-yl)dihydropyrimidine-2,4(1H,3H)-dione, TFA (C-154, 77 mg, 0.185 mmol) was charged with 1-(4-(((R)-1-cyanoethyl)amino)-5-(4-(trans-4-(2-oxoethyl)cyclohexyl)-1H-1,2,3-triazol-1-yl)pyridin-2-yl)-1H-pyrazolo[3,4-b]pyridine-5-carbonitrile (A-57, 0.1 M in DMSO, 1.5 mL, mmol), triethylamine (0.525 mL, 0.375 mmol), and DMSO (0.5 mL). This solution was stirred at room temperature for 15 min fore the addition of sodium triacetoxyborohydride (95 mg, 0.45 mmol). This reaction mixture was stirred at room temperature for 40 hours, whereupon LCMS analysis indicated incomplete conversion. Additional triethylamine (0.525 mL, 0.375 mmol) and sodium triacetoxyborohydride (159 mg, 0.75 mmol) was added and the temperature was increased to 35° C. The reaction was stirred at temperature for 3 days then allowed to cool to room temperature and filtered to remove residual solids. The crude material was purified via preparative reverse phase chromatography with the following conditions: Column: XBridge C18, 19 mm×200 mm, 5 μm particles; Mobile Phase A: ACN/H₂O (5:95) with 10 mM AA; Mobile Phase B: ACN/H₂O (95:5) with 10 mM AA; Temperature: 25° C.; Gradient: 22-62% B (0.0-20.0 min), 100% B (20.0-24.0 min); Flow: 20.0 mL/min; Detection: UV (220 nm) and MS (ESI

Fractions containing the desired product were combined and dried via centrifugal evaporation. The material was further purified via preparative Reverse Phase with the following conditions: Column: XBridge C18, 19 mm×200 mm, 5 μm particles; Mobile Phase A: ACN/H₂O (5:95) with 0.1% TFA; Mobile Phase B: ACN/H₂O (95:5) with 0.1% TFA; Temperature: 25° C.; Gradient: 6-46% B (0.0-20.0 min), 100% B (20.0-24.0 min); Flow: 20.0 mL/min; Detection: UV (220 nm) and MS (ESI+).

Fractions containing the desired product were combined and dried via centrifugal evaporation to afford 1-(4-(((R)-1-cyanoethyl)amino)-5-(4-(trans-4-(2-((R)-4-(5-(2,4-dioxotetrahydropyrimidin-1(2H)-yl)pyridin-2-yl)-3-ethylpiperazin-1-yl)ethyl)cyclohexyl)-1H-1,2,3-triazol-1-yl)pyridin-2-yl)-1H-pyrazolo[3,4-b]pyridine-5-carbonitrile (P-394, 42.6 mg, 37.0% yield) as an off-white solid [M+H]⁺=768.3. ¹H-NMR (500 MHz, DMSO-d6) δ 10.56 (s, 1H), 9.53 (s, 1H), 9.07 (br d, J=6.2 Hz, 2H), 8.72 (s, 1H), 8.61 (s, 1H), 8.42 (s, 1H), 8.38 (s, 1H), 7.69 (s, 1H), 7.40 (d, J=7.3 Hz, 1H), 7.26-7.18 (m, 2H), 6.86-6.78 (m, 1H), 5.03 (br t, J=7.5 Hz, 1H), 3.97-3.89 (m, 1H), 3.75-3.53 (m, 3H), 3.17 (s, 1H), 2.98 (br d, J=9.9 Hz, 1H), 2.82-2.73 (m, 2H), 2.55 (s, 5H), 2.18 (br d, J=13.2 Hz, 2H), 1.93 (br d, J=12.6 Hz, 2H), 1.70 (br s, 2H), 1.62 (d, J=6.9 Hz, 3H), 1.58-1.40 (m, 3H), 1.27-1.14 (m, 3H).

### Example S93. Preparation of (R)-1-(5-azido-4-((1-cyanoethyl)amino)pyridin-2-yl)-1H-pyrazolo[3,4-b]pyridine-5-carbonitrile-1-(4-(((R)-1-cyanoethyl)amino)-5-(4-(4-(4-(4-(2,4-dioxotetrahydropyrimidin-1(2H)-yl)phenyl)piperazin-1-yl)cyclohexyl)-1H-1,2,3-triazol-1-yl)pyridin-2-yl)-1H-pyrazolo[3,4-b]pyridine-5-carbonitrile-1-(4-(4-(4-ethynylcyclohexyl)piperazin-1-yl)phenyl)dihydropyrimidine-2,4(1H,3H)-dione (1/1/1) (P-499)

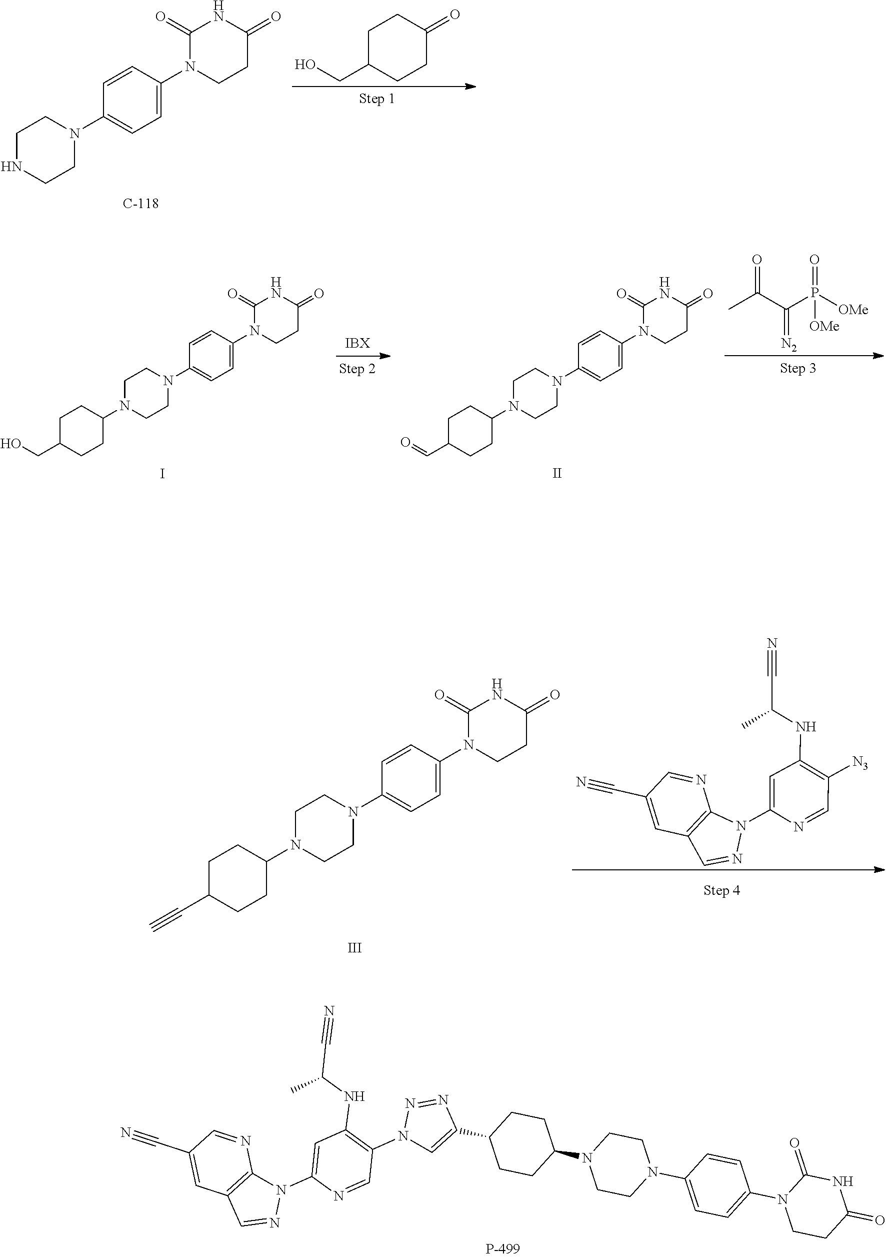

Step 1: Preparation of 1-(4-(4-(4-(hydroxymethyl)cyclohexyl)piperazin-1-yl)phenyl)dihydropyrimidine-2,4-(1H,3H)-dione (I). To a stirred solution of 1-(4-(piperazin-1-yl)phenyl)dihydropyrimidine-2,4(1H,3H)-dione, 2 TFA (C-118, 2.510 g, 4.37 mmol) and 4-(hydroxymethyl)cyclohexan-1-one (800 mg, 6.24 mmol) was added DCE/ethanol (9:1, 22 mL). The pH of the reaction was adjusted to pH 5-6 by adding DIPEA (0.545 mL, 3.12 mmol). The resulting reaction mixture was stirred at 50° C. for 30 min. MP(CN)BH₃ (784 mg, 12.48 mmol) was added to the reaction mixture at the same temperature, and the resulting reaction mixture was stirred at 50° C. for 10 h. The reaction mixture was cooled to room temperature and diluted with DCM (15 mL). The resulting reaction mixture was filtered through a celite bed and washed with 10% methanol/DCM (45 mL). The filtrate was washed with saturated NaHCO₃ solution (50 mL) followed by brine (25 mL). The organic extract was dried over sodium sulfate, filtered and concentrated under reduced pressure to get crude compound. The crude compound was triturated with diethyl ether (3×10 mL) and dried under vacuum to get 1-(4-(4-(4-(hydroxymethyl) cyclohexyl)piperazin-1-yl)phenyl)dihydropyrimidine-2,4(1H,3H)-dione (I, 1.15 g, 35% yield) as pale orange solid. It was used for the next step without further purification. [M+H]⁺=387.2.

Step 2: Preparation of 4-(4-(4-(2,4-dioxotetrahydropyrimidin-1(2H)-yl)phenyl)piperazin-1-yl)cyclohexane-1-carbaldehyde (II). To a stirred solution of 1-(4-(4-(4-(hydroxymethyl)cyclohexyl)piperazin-1-yl)phenyl)dihydro pyrimidine-2,4(1H,3H)-dione (I, 1.1 g, 2.049 mmol) in DMSO (10 mL) was added IBX (2.295 g, 8.20 mmol) at room temperature. The resulting reaction mixture was stirred at room temperature for 4 h. The reaction mixture was diluted with ethyl acetate (50 mL) and separated with saturated NaHCO₃ solution (2×50 mL), followed by brine solution (20 mL). The organic extract was dried over sodium sulfate, filtered, and concentrated under reduced pressure to get crude compound. The crude compound was triturated with diethyl ether (3×10 mL) to get 4-(4-(4-(2,4-dioxotetrahydropyrimidin-1(2H)-yl)phenyl)piperazin-1-yl)cyclohexane-1-carbaldehyde (II, 800 mg, 91% yield) as pale orange colour solid. It was used for the next step without further purification. [M+H]⁺=385.2.

Step 3: Preparation of 1-(4-(4-(4-ethynylcyclohexyl)piperazin-1-yl)phenyl)dihydropyrimidine-2,4(1H,3H)-dione (III). To a stirred ice-cold solution of 4-(4-(4-(2,4-dioxotetrahydropyrimidin-1(2H)-yl)phenyl)piperazin-1-yl)cyclohexane-1-carbaldehyde (II, 800 mg, 1.873 mmol) in methanol (10 mL) was added K₂CO₃ (1.032 g, 7.49 mmol). Dimethyl (1-diazo-2-oxopropyl)phosphonate (899 mg, 4.68 mmol) was added to the reaction mixture dropwise. The resulting reaction mixture was warmed up to room temperature and stirred for 3 h. The reaction mixture was poured into ice-cold water (100 mL) and stirred for 10 min at room temperature. The obtained solid was filtered and washed with water (20 mL). The solid was dried under vacuum for 16 h at room temperature to obtain 1-(4-(4-(4-ethynylcyclohexyl)piperazin-1-yl)phenyl)dihydropyrimidine-2,4(1H,3H)-dione (III, 450 mg, 49% yield) as pale yellow solid. It was used for the next step without further purification. [M+H]⁺=381.2.

Step 4: Preparation of 1-(4-(((R)-1-cyanoethyl)amino)-5-(4-(4-(4-(4-(2,4-dioxotetrahydropyrimidin-1-(2H)-yl)phenyl)piperazin-1-yl)cyclohexyl)-1H-1,2,3-triazol-1-yl)pyridin-2-yl)-1H-pyrazolo[3,4-b]pyridine-5-carbonitrile (P-499). To a stirred solution of 1-(4-(4-(4-ethynylcyclohexyl)piperazin-1-yl)phenyl)dihydropyrimidine-2,4(1H,3H)-dione (III, 540 mg, 1.106 mmol) and (R)-1-(5-azido-4-((1-cyanoethyl)amino)pyridin-2-yl)-1H-pyrazolo[3,4-b]pyridine-5-carbonitrile (600 mg, 1.580 mmol) in acetonitrile (20 mL) was added sodium ascorbate (125 mg, 0.632 mmol). A solution of Cu(SO₄)₂·5H₂O (158 mg, 0.632 mmol) in water (4 mL) was added dropwise into the reaction mixture over a period of 2 min. The resulting reaction mixture was stirred at room temperature for 16 h. The reaction mixture was treated with water (100 mL), filtered through celite bed, and the celite bed was washed with 10% methanol/DCM (100 mL). The filtrate was separated using water and extracted with 10% methanol/DCM (2×50 mL). The combined organic extracts were dried over sodium sulfate, filtered, and concentrated under reduced pressure to get crude compound. The crude compound was suspended into 10% methanol/DCM (10 mL), heated to 40° C. to get a clear solution before acetonitrile (50 mL) was added dropwise into the reaction mixture under stirring. The obtained residue was stirred for 30 min at room temperature, filtered, and washed with acetonitrile (10 mL). The obtained solid was dried under vacuum for 16 h to obtain 1-(4-(((R)-1-cyanoethyl)amino)-5-(4-(4-(4-(4-(2,4-dioxotetrahydropyrimidin-1(2H)-yl)phenyl)piperazin-1-yl)cyclohexyl)-1H-1,2,3-triazol-1-yl)pyridin-2-yl)-1H-pyrazolo[3,4-b]pyridine-5-carbonitrile (P-499, 150 mg, 13% yield) as a pink color solid. [M+H]⁺=711.5.

Table 71 summarizes the final compounds that were prepared using General Procedure X-15.

TABLE 71
Final Compounds Prepared using General Procedure X-15

| Com- | | | | |
|---|---|---|---|---|
| pound | | | | |
| No. | TBM | CBM | Structure | Characterization |
| P-344 | A-57 | C-117 | 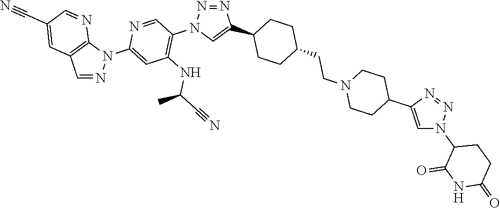 [SMILES: C[C@H](C#N)Nc1cc(-n2ncc3cc(C#N)cnc32)ncc1-n1cc([C@H]2CC[C@H](CCN3CCC(c4cn(C5CCC(=O)NC5=O)nn4)CC3)CC2)nn1] | 22.7% yield [M + H]⁺ = 728.3 ¹H-NMR not furnished |
| P-366 | A-57 | C-118 | 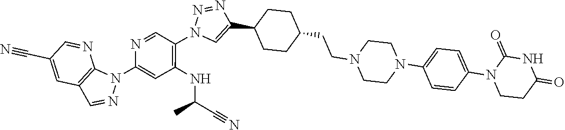 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)cc4)CC3)CC2)nn1] | 14.2% yield [M + H]⁺ = 739.3 1¹H-NMR: (400 MHz, DMSO-d₆) δ ppm: 1.12-1.28 (m, 2H), 1.35-1.59 (m, 3H), 1.62 (d, J = 7.2 Hz, 3H), 1.65-1.68 (m, 2H), 1.83-1.93 (m, 2H), 2.11-2.22 (m, 2H), |
| | | | | 2.65-2.82 (m, 3H), 2.91-3.07 |
| | | | | (m, 2H), 3.10-3.30 (m, 4H), |
| | | | | 3.61-3.64 (m, 2H), 3.72 (t, |
| | | | | J = 6.8 Hz, 2H), 3.85-3.88 |
| | | | | (m, 2H), 4.98-5.10 (m, 1H), |
| | | | | 7.03-7.05 (m, 2H), 7.19- |
| | | | | 7.24 (m, 3H), 7.68 (s, 1H), |
| | | | | 8.37 (s, 1H), 8.42 (s, 1H), |
| | | | | 8.73 (s, 1H), 9.06-9.08 (m, |
| | | | | 2H), 10.29 (s, 1H). |
| P-340 | A-57 | C-119 | 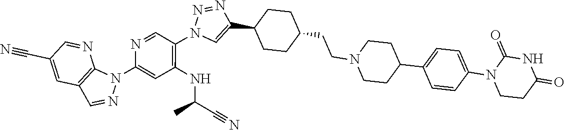 [SMILES: C[C@H](C#N)Nc1cc(-n2ncc3cc(C#N)cnc32)ncc1-n1cc([C@H]2CC[C@H](CCN3CCC(c4ccc(N5CCC(=O)NC5=O)cc4)CC3)CC2)nn1] | 12.2% yield [M + H]⁺ = 738.3 ¹H-NMR (500 MHz, DMSO- d₆) Shift 10.39 (s, 1H), 9.12- 9.08 (m, 2H), 8.76 (s, 1H), 8.46 (s, 1H), 8.40 (s, 1H), 7.72 (s, 1H), 7.33-7.23 (m, 5H), 5.06 (quin, J = 7.4 Hz, |
| | | | | 1H), 3.81 (t, J = 6.6 Hz, 2H), |
| | | | | 3.51-3.35 (m, 1H), 3.19-3.05 |
| | | | | (m, 1H), 2.82-2.72 (m, 3H), |
| | | | | 2.19 (br d, J = 12.7 Hz, 2H), |
| | | | | 1.99-1.63 (m, 10H), 1.58- |
| | | | | 1.39 (m, 5H), 1.19 (q, J = |
| | | | | 13.0 Hz, 2H) |
| | | | | Four protons are not apparent |
| P-355 | A-57 | C-120 | 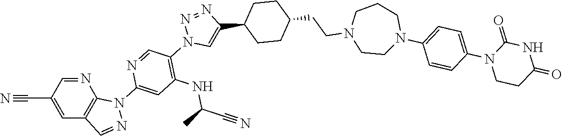 [SMILES: C[C@H](C#N)Nc1cc(-n2ncc3cc(C#N)cnc32)ncc1-n1cc([C@H]2CC[C@H](CCN3CCCN(c4ccc(N5CCC(=O)NC5=O)cc4)CC3)CC2)nn1] | 6.8% yield [M + H]⁺ = 753.1 ¹H-NMR (500 MHz, DMSO- d₆) Shift 10.28 (s, 1H), 9.12- 9.09 (m, 2H), 8.76 (s, 1H), 8.45 (s, 1H), 8.40 (s, 1H), 7.72 (s, 1H), 7.30-7.16 (m, 4H), 6.82 (d, J = 8.8 Hz, 1H), 5.06 (quin, J = 7.4 Hz, 1H), |
| | | | | 3.99-3.80 (m, 1H), 3.72 (t, |
| | | | | J = 6.6 Hz, 2H), 3.32-3.18 (m, |
| | | | | 1H), 2.83-2.71 (m, 3H), 2.31- |
| | | | | 2.10 (m, 4H), 1.90 (br d, J = |
| | | | | 11.4 Hz, 2H), 1.71-1.62 (m, |
| | | | | 5H), 1.60-1.36 (m, 4H), |
| | | | | 1.22 (q, J = 12.0 Hz, 2H) |
| | | | | Three protons are not |
| | | | | apparent |
| P-354 | A-57 | C-121 | 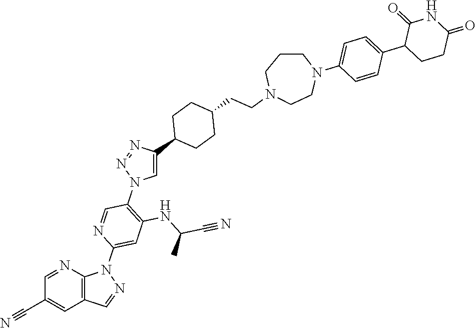 [SMILES: C[C@H](C#N)Nc1cc(-n2ncc3cc(C#N)cnc32)ncc1-n1cc([C@H]2CC[C@H](CCN3CCCN(c4ccc(C5CCC(=O)NC5=O)cc4)CC3)CC2)nn1] | 11.7% yield [M + H]⁺ = 753.3 ¹H-NMR (500 MHz, DMSO- d₆) Shift 10.78 (s, 1H), 9.12- 9.08 (m, 2H), 8.76 (s, 1H), 8.45 (s, 1H), 8.39 (s, 1H), 7.71 (s, 1H), 7.24 (d, J = 8.3 Hz, 1H), 7.02 (d, J = 8.7 Hz, 2H), 6.69 (d, J = 8.5 Hz, 2H), 5.05 (quin, J = 7.5 Hz, 1H), 3.71 (dd, J = 10.6, 5.0 Hz, 1H), 3.57-3.50 (m, 1H), 3.50- 3.44 (m, 1H), 2.83-2.62 (m, 4H), 2.16 (br d, J = 10.2 Hz, 3H), 2.11-2.01 (m, 1H), 1.98- 1.85 (m, 6H), 1.66 (d, J = 7.0 Hz, 4H), 1.56-1.35 (m, 5H), 1.15 (q, J = 12.7 Hz, 2H) Two protons are not apparent |
| P-368 | A-57 | C-122 | 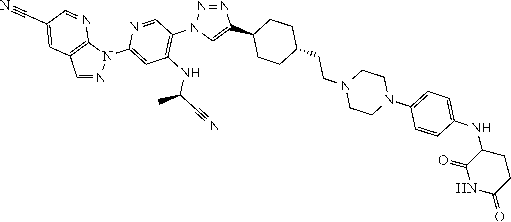 [SMILES: C[C@H](C#N)Nc1cc(-n2ncc3cc(C#N)cnc32)ncc1-n1cc([C@H]2CC[C@H](CCN3CCN(c4ccc(NC5CCC(=O)NC5=O)cc4)CC3)CC2)nn1] | 37.7% yield [M + H]⁺ = 753.2 ¹H-NMR (500 MHz, DMSO- d₆) Shift 10.82 (s, 1H), 9.14- 9.09 (m, 2H), 8.77 (s, 1H), 8.46 (s, 1H), 8.41 (s, 1H), 7.72 (s, 1H), 7.24 (br d, J = 8.2 Hz, 1H), 6.88 (br d, J = 9.0 Hz, 2H), 6.72 (br d, J = 8.7 Hz, 2H), 5.07 (quin, J = 7.4 Hz, 1H), 4.29 (dd, J = 11.3, 4.6 Hz, 1H), 3.67-3.58 (m, 1H), 3.33-3.15 (m, 2H), 2.95-2.87 (m, 1H), 2.85-2.74 |
| | | | | (m, 2H), 2.69-2.58 (m, 2H), |
| | | | | 2.24-2.11 (m, 3H), 1.97-1.86 |
| | | | | (m, 3H), 1.72-1.63 (m, 5H), |
| | | | | 1.61-1.48 (m, 2H), 1.45 (br s, |
| | | | | 1H), 1.29-1.14 (m, 2H) |
| | | | | Five protons are not apparent |
| P-348 | A-57 | C-123 | 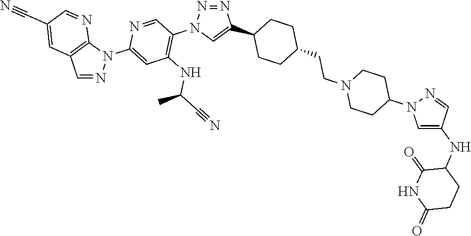 [SMILES: C[C@H](C#N)Nc1cc(-n2ncc3cc(C#N)cnc32)ncc1-n1cc([C@H]2CC[C@H](CCN3CCC(n4cc(NC5CCC(=O)NC5=O)cn4)CC3)CC2)nn1] | 10.1% yield [M + H]⁺ = 742.2 ¹H-NMR (500 MHz, DMSO- d₆) Shift 10.81 (s, 1H), 9.11 (dd, J = 8.8, 1.9 Hz, 2H), 8.76 (s, 1H), 8.46 (s, 1H), 8.40 (s, 1H), 7.72 (s, 1H), 7.33 (s, 1H), 7.25 (d, J = 8.1 Hz, 1H), 7.11 (s, 1H), 5.06 (quin, J = 7.4 Hz, 1H), 4.08- 3.98 (m, 1H), 3.86-3.80 (m, 1H), 3.12-2.99 (m, 1H), 2.82-2.65 (m, 2H), 2.26-2.08 (m, 4H), 2.02-1.80 (m, 8H), 1.66 (d, J = 7.0 Hz, 3H), |
| | | | | 1.58-1.36 (m, 2H), 1.17 (q, |
| | | | | J = 11.1 Hz, 2H) |
| | | | | Five protons are not apparent |
| P-365 | A-57 | C-124 | 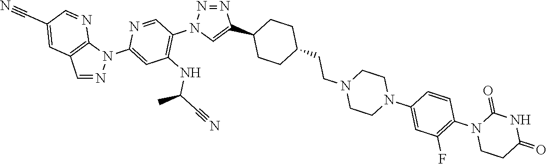 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)c(F)c4)CC3)CC2)nn1] | 4% yield [M + H]⁺ = 757.3 ¹H-NMR (500 MHz, DMSO- d₆) Shift 10.52-10.30 (m, 1H), 9.11 (dd, J = 9.1, 1.9 Hz, 2H), 8.76 (s, 1H), 8.45 (s, 1H), 8.40 (s, 1H), 7.72 (s, 1H), 7.30-7.22 (m, 2H), 6.87 (d, J = 13.7 Hz, 1H), 6.82 (d, J = 9.0 Hz, 1H), 5.06 (quin, |
| | | | | J = 7.5 Hz, 1H), 3.67 (t, J = |
| | | | | 6.7 Hz, 2H), 3.46-3.38 (m, |
| | | | | 1H), 3.24 (br s, 1H), 2.82- |
| | | | | 2.68 (m, 3H), 2.53-2.36 (m, |
| | | | | 2H), 2.18 (br d, J = 11.2 Hz, |
| | | | | 2H), 1.97-1.88 (m, 2H), 1.66 |
| | | | | (d, J = 6.9 Hz, 3H), 1.59-1.36 |
| | | | | (m, 2H), 1.19 (q, J = 11.7 Hz, |
| | | | | 2H) |
| | | | | Six protons are not apparent |
| | | | | ¹⁹F-NMR (470 MHz, DMSO- |
| | | | | d₆): δ ppm −120.28 |
| P-343 | A-57 | C-125 | 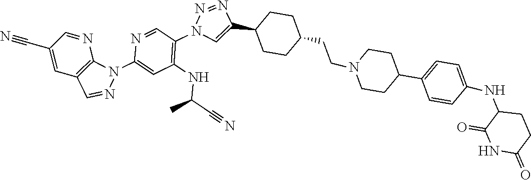 [SMILES: C[C@H](C#N)Nc1cc(-n2ncc3cc(C#N)cnc32)ncc1-n1cc([C@H]2CC[C@H](CCN3CCC(c4ccc(NC5CCC(=O)NC5=O)cc4)CC3)CC2)nn1] | 4% yield [M + H]⁺ = 752.1 ¹H-NMR (500 MHz, DMSO- d₆) Shift 9.11 (dd, J = 8.6, 1.9 Hz, 2H), 8.76 (s, 1H), 8.46 (s, 1H), 8.40 (s, 1H), 7.72 (s, 1H), 7.24 (d, J = 8.5 Hz, 1H), 7.06 (br d, J = 8.5 Hz, 2H), 6.73 (br d, J = 8.8 Hz, 2H), 5.06 (quin, J = 7.4 Hz, 1H), 4.79 (br dd, J = 12.8, 4.7 Hz, 1H), 3.95 (s, 1H), 3.26-3.10 |
| | | | | (m, 2H), 2.91-2.72 (m, 3H), |
| | | | | 2.46-2.32 (m, 1H), 2.19 (br d, |
| | | | | J = 9.5 Hz, 2H), 2.02-1.90 (m, |
| | | | | 5H), 1.86-1.72 (m, 2H), 1.71- |
| | | | | 1.39 (m, 10H), 1.31-1.15 (m, |
| | | | | 2H) |
| | | | | Four protons are not apparent |
| P-363 | A-57 | C-126 | 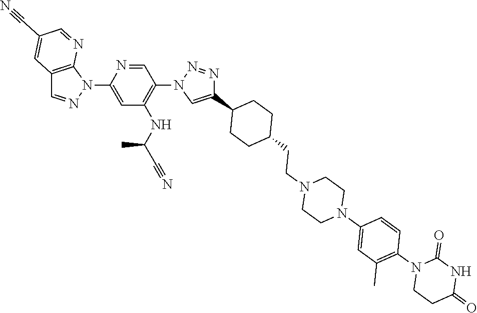 [SMILES: Cc1cc(N2CCN(CC[C@H]3CC[C@H](c4cn(-c5cnc(-n6ncc7cc(C#N)cnc76)cc5N[C@H](C)C#N)nn4)CC3)CC2)ccc1N1CCC(=O)NC1=O] | 47.7% yield [M + H]⁺ = 753.4 ¹H-NMR (400 MHz, DMSO- d₆): δ ppm 1.18-1.27 (m, 2H), 1.35-1.59 (m, 3H), 1.63 (d, J = 6.8 Hz, 3H), 1.65-1.67 (m, 2H), 1.88-1.91 (m, 2H), 2.16 (s, 3H), 2.15-2.21 (m, 2H), 2.68-2.77 (m, 3H), 2.95- 3.03 (m, 2H), 3.12-3.30 (m, 4H), 3.46-3.52 (m, 1H), 3.61- 3.72 (m, 3H), 3.86-3.89 (m, 2H), 4.98-5.06 (m, 1H), 6.90- 6.93 (m, 2H), 7.11-7.23 (m, 2H), 7.68 (s, 1H), 8.36 (s, 1H), 8.41 (s, 1H), 8.73 (s, 1H), 9.06-9.08 (m, 2H), 10.29 (s, 1H) |
| P-347 | A-57 | C-127 | 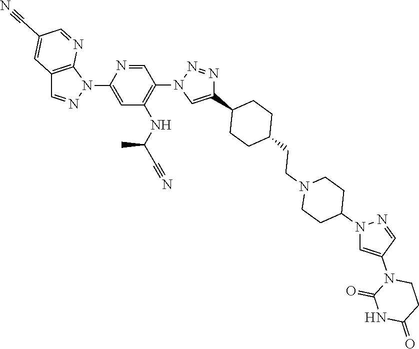 [SMILES: C[C@H](C#N)Nc1cc(-n2ncc3cc(C#N)cnc32)ncc1-n1cc([C@H]2CC[C@H](CCN3CCC(n4cc(N5CCC(=O)NC5=O)cn4)CC3)CC2)nn1] | 21.7% yield [M + H]⁺ = 728.4 ¹H-NMR (400 MHz, DMSO- d₆): δ ppm 1.10-1.30 (m, 2H), 1.35-1.60 (m, 7H), 1.88- 1.91 (m, 2H), 2.10-2.40 (m, 5H), 2.65-2.85 (m, 3H), 3.05- 3.30 (m, 4H), 3.68-3.80 (m, 4H), 4.40-4.55 (m, 1H), 5.01- 5.05 (m, 1H), 7.19-7.22 (m, 1H), 7.67-7.71 (m, 2H), 8.00 (s, 1H), 8.38 (s, 1H), 8.42 (s, 1H), 8.73 (s, 1H), 9.07-9.08 (m, 2H), 10.41 (s, 1H). Two proton are not apparent |
| P-358 | A-57 | C-128 | 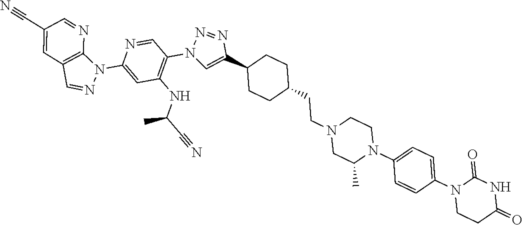 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)cc4)[C@H](C)C3)CC2)nn1] | 49.1% yield [M + H]⁺ = 753.3 ¹H-NMR (400 MHz, DMSO- d₆, 100 oC): δ ppm 1.04-1.20 (brs, 3H), 1.22-1.28 (m, 2H), 1.45-1.59 (m, 3H), 1.67 (d, J = 6.8 Hz, 3H), 1.68-1.72 (m, 2H), 1.93-1.95 (m, 2H), 2.19- 2.22 (m, 2H), 2.72 (t, J = 6.8 Hz, 2H), 2.79-2.85 (m, 1H), 3.23-3.30 (m, 2H), 3.73-3.77 (m, 2H), 4.91-4.99 (m, 1H), 7.02-7.08 (m, 3H), 7.28 (m, 2H), 7.75 (s, 1H), 8.31 (s, |
| | | | | 1H), 8.46 (s, 1H), 8.66 (s, |
| | | | | 1H), 8.97-9.01 (m, 2H), |
| | | | | 9.88 (s, 1H). |
| | | | | Seven protons are not |
| | | | | apparent |
| P-353 | A-57 | C-129 | 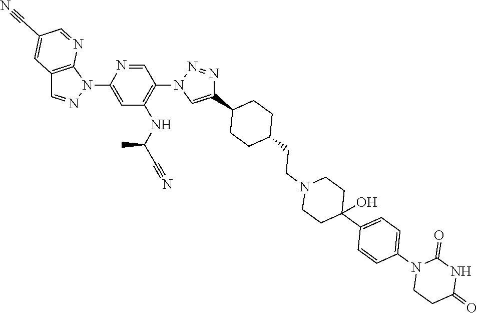 [SMILES: C[C@H](C#N)Nc1cc(-n2ncc3cc(C#N)cnc32)ncc1-n1cc([C@H]2CC[C@H](CCN3CCC(O)(c4ccc(N5CCC(=O)NC5=O)cc4)CC3)CC2)nn1] | 1.1% yield [M + H]⁺ = 754.4 ¹H-NMR not furnished |
| P-356 | A-57 | C-130 | 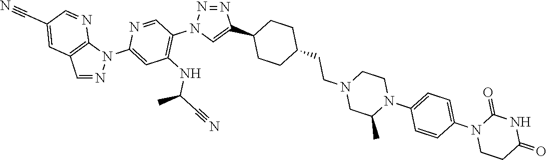 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)cc4)[C@@H](C)C3)CC2)nn1] | 1.4% yield [M + H]⁺ = 753.4 ¹H-NMR not furnished |
| P-364 | A-57 | C-131 | 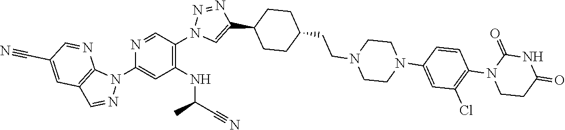 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)c(Cl)c4)CC3)CC2)nn1] | 48.3% yield [M + H]⁺ = 773.2 ¹H-NMR (400 MHz, DMSO- d₆): δ ppm 1.10-1.27 (m, 2H), 1.31-1.59 (m, 3H), 1.61 (d, J = 8.0 Hz, 3H), 1.62- 1.70 (m, 2H), 1.85-1.95 (m, 2H), 2.12-2.20 (m, 2H), |
| | | | | 2.65-2.83 (m, 3H), 3.02-3.20 |
| | | | | (m, 4H), 3.21-3.30 (m, 2H), |
| | | | | 3.50-3.60 (m, 3H), 3.90-4.00 |
| | | | | (m, 2H), 4.98-5.10 (m, 1H), |
| | | | | 7.04-7.07 (m, 1H), 7.18-7.20 |
| | | | | (m, 2H), 7.34-7.36 (m, 1H), |
| | | | | 7.68 (s, 1H), 8.37 (s, 1H), |
| | | | | 8.42 (s, 1H), 8.73 (s, 1H), |
| | | | | 9.06-9.08 (m, 2H), 10.40 (s, |
| | | | | 1H). |
| | | | | one proton is not apparent |
| P-412 | A-13 | C-118 | 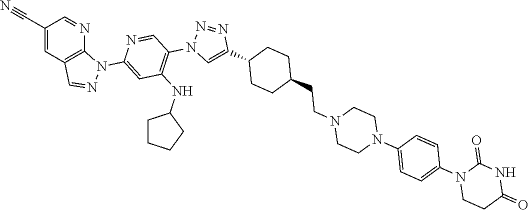 [SMILES: N#Cc1cnc2c(cnn2-c2cc(NC3CCCC3)c(-n3cc([C@H]4CC[C@H](CCN5CCN(c6ccc(N7CCC(=O)NC7=O)cc6)CC5)CC4)nn3)cn2)c1] | 39.7% yield [M + H]⁺ = 754.4 ¹H-NMR (400 MHz, DMSO- d₆) δ ppm 1.12-1.22 (m, 2H), 1.37-1.68 (m, 11H), 1.87-1.90 (m, 2H), 2.04-2.08 (m, 2H), 2.13-2.16 (m, 2H), 2.67-2.79 (m, 3H), 2.95-3.02 (m, 2H), 3.11-3.17 (m, 2H), 3.23-3.26 (m, 2H), 3.60-3.63 (m, 2H), 3.70-3.73 (m, 2H), 3.84-3.70 (m, 2H), 3.92-3.97 (m, 1H), 6.81-6.83 (m, 1H), 7.03 (d, |
| | | | | J = 9.2 Hz, 2H), 7.23 (d, J = |
| | | | | 8.8 Hz, 2H), 7.56 (s, 1H), |
| | | | | 8.34 (s, 1H), 8.44 (s, 1H), |
| | | | | 8.67 (s, 1H), 9.02-9.07 (m, |
| | | | | 2H), 10.29 (s, 1H). |
| P-335 | A-57 | C-133 | 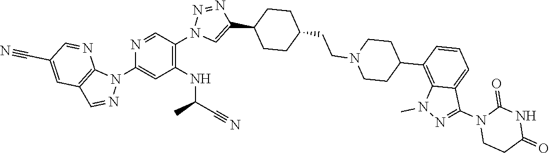 [SMILES: C[C@H](C#N)Nc1cc(-n2ncc3cc(C#N)cnc32)ncc1-n1cc([C@H]2CC[C@H](CCN3CCC(c4cccc5c(N6CCC(=O)NC6=O)nn(C)c45)CC3)CC2)nn1] | 50.1% yield [M + H]⁺ = 792.3 ¹H-NMR (500 MHz, DMSO- d₆) Shift 10.60 (s, 1H), 9.11 (d, J = 6.9 Hz, 2H), 8.77 (s, 1H), 8.46 (s, 1H), 8.42 (s, 1H), 7.73 (s, 1H), 7.57 (d, J = 8.0 Hz, 1H), 7.30-7.22 (m, 2H), 7.21-7.14 (m, 1H), 5.07 (quin, J = 7.6 Hz, 1H), 4.30 |
| | | | | (s, 3H), 3.96-3.91 (m, 3H), |
| | | | | 3.83-3.65 (m, 2H), 3.56-3.40 |
| | | | | (m, 1H), 3.35-3.18 (m, 3H), |
| | | | | 2.82 (br t, J = 6.6 Hz, 3H), |
| | | | | 2.22 (br d, J = 12.9 Hz, 4H), |
| | | | | 2.10-1.89 (m, 4H), 1.77-1.63 |
| | | | | (m, 5H), 1.62-1.43 (m, 3H), |
| | | | | 1.25 (q, J = 11.4 Hz, 2H) |
| P-324 | A-57 | C-134 | 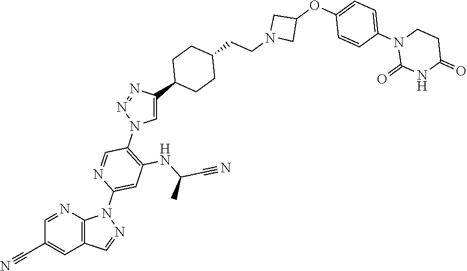 [SMILES: C[C@H](C#N)Nc1cc(-n2ncc3cc(C#N)cnc32)ncc1-n1cc([C@H]2CC[C@H](CCN3CC(Oc4ccc(N5CCC(=O)NC5=O)cc4)C3)CC2)nn1] | 2.1% yield [M + H]⁺ = 726.3 ¹H-NMR (500 MHz, DMSO- d₆) Shift 10.38 (s, 1H), 9.13- 9.08 (m, 2H), 8.76 (s, 1H), 8.45 (s, 1H), 8.40 (s, 1H), 7.72 (s, 1H), 7.37-7.31 (m, 2H), 7.24 (d, J = 8.0 Hz, 1H), 7.00-6.91 (m, 2H), 5.24-4.99 (m, 2H), 4.76 (br s, 1H), 4.52 (br s, 1H), 4.31 (br s, 1H), 4.18 (br s, 1H), 3.95 (s, 1H), 3.77 (t, J = 6.4 Hz, 2H), 2.94 (s, 1H), 2.80-2.73 (m, 3H), 2.19 (br d, J = 12.5 Hz, 2H), 1.89 (br d, J = 9.7 Hz, 2H), 1.65 (d, J = 7.0 Hz, 3H), 1.58- |
| | | | | 1.40 (m, 5H), 1.19 (q, J = |
| | | | | 11.9 Hz, 2H) |
| P-342 | A-57 | C-135 | 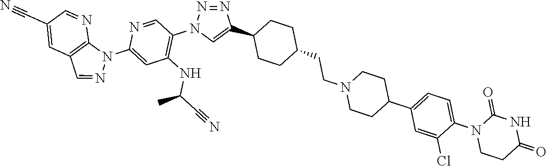 [SMILES: C[C@H](C#N)Nc1cc(-n2ncc3cc(C#N)cnc32)ncc1-n1cc([C@H]2CC[C@H](CCN3CCC(c4ccc(N5CCC(=O)NC5=O)c(Cl)c4)CC3)CC2)nn1] | 23.8% yield [M + H]⁺ = 772.4 ¹H-NMR (400 MHz, DMSO- d₆): δ ppm 1.12-1.22 (m, 2H), 1.38-1.42 (m, 1H), 1.47- 1.56 (m, 2H), 1.62 (d, J = 6.8 Hz, 3H), 1.65-1.68 (m, 2H), 1.81-1.93 (m, 4H), 2.08-2.19 (m, 4H), 2.71-2.80 (m, 3H), 2.89-2.95 (m, 1H), 3.01-3.10 (m, 2H), 3.17-3.22 (m, 2H), |
| | | | | 3.32-3.41 (m, 1H), 3.55-3.75 |
| | | | | (m, 3H), 4.99-5.06 (m, 1H), |
| | | | | 7.20 (d, J = 8.0 Hz, 1H), |
| | | | | 7.30-7.32 (m, 1H), 7.46-7.49 |
| | | | | (m, 2H), 7.68 (s, 1H), 8.38 (s, |
| | | | | 1H), 8.42 (s, 1H), 8.73 (s, |
| | | | | 1H), 9.06-9.08 (m, 2H), 10.47 |
| | | | | (s, 1H). |
| P-345 | A-57 | C-136 | 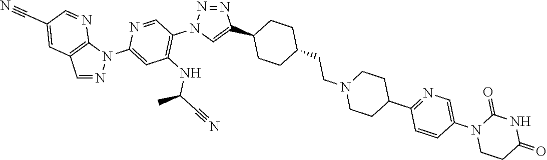 [SMILES: C[C@H](C#N)Nc1cc(-n2ncc3cc(C#N)cnc32)ncc1-n1cc([C@H]2CC[C@H](CCN3CCC(c4ccc(N5CCC(=O)NC5=O)cn4)CC3)CC2)nn1] | 49.3% yield [M + H]⁺ = 739.3 ¹H-NMR (400 MHz, DMSO- d₆): δ ppm 1.12-1.22 (m, 2H), 1.40-1.56 (m, 3H), 1.62 (d, J = 6.8 Hz, 3H), 1.65- 1.68 (m, 2H), 1.88-1.91 (m, 2H), 1.98-2.02 (m, 2H), 2.08- 2.17 (m, 4H), 2.72-2.79 (m, 3H), 2.99-3.11 (m, 2H), 3.15- |
| | | | | 3.30 (m, 2H), 3.82-3.85 (m, |
| | | | | 2H), 4.99-5.04 (m, 1H), 7.20 |
| | | | | (d, J = 8.0 Hz, 1H), 7.38 (d, |
| | | | | J = 8.4 Hz, 1H), 7.68 (s, 1H), |
| | | | | 7.66-7.81 (m, 1H), 8.37-8.42 |
| | | | | (m, 2H), 8.55-8.54 (m, 1H), |
| | | | | 8.73-8.72 (m, 1H), 9.06-9.17 |
| | | | | (m, 2H), 10.50 (s, 1H). |
| | | | | Three protons are not |
| | | | | apparent |
| P-360 | A-57 | C-137 | 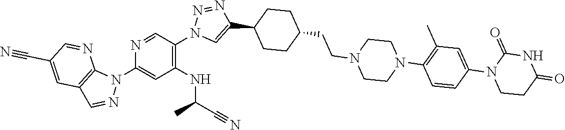 [SMILES: Cc1cc(N2CCC(=O)NC2=O)ccc1N1CCN(CC[C@H]2CC[C@H](c3cn(-c4cnc(-n5ncc6cc(C#N)cnc65)cc4N[C@H](C)C#N)nn3)CC2)CC1] | 19.9% yield [M + H]⁺ = 753.2 ¹H-NMR (400 MHz, DMSO d6): δ ppm 1.13-1.22 (m, 2H), 1.36-1.58 (m, 3H), 1.62 (d, J = 6.8 Hz, 3H), 1.65-1.69 (m, 2H), 1.89-1.92 (m, 2H), 2.16- 2.18 (m, 2H), 2.28 (s, 3H), 2.68-2.70 (m, 2H), 2.73-2.81 |
| | | | | (m, 1H), 2.93-3.00 (m, 2H), |
| | | | | 3.21-3.25 (m, 6H), 3.59-3.62 |
| | | | | (m, 2H), 3.71-3.75 (m, 2H), |
| | | | | 4.99-5.50 (m, 1H), 7.12-7.21 |
| | | | | (m, 4H), 7.68 (s, 1H), 8.37 (s, |
| | | | | 1H), 8.41 (s, 1H), 8.72 (s, |
| | | | | 1H), 9.05-9.07 (m, 2H), 10.32 |
| | | | | (s, 1H). |
| P-367 | A-57 | C-138 | 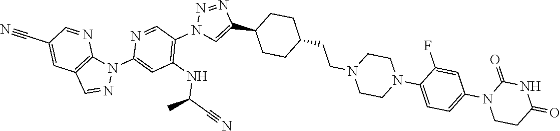 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)cc4F)CC3)CC2)nn1] | 1.1% yield [M + H]⁺ = 757.4 ¹H-NMR (500 MHz, DMSO- d₆) Shift 10.41 (s, 1H), 9.12- 9.08 (m, 2H), 8.76 (s, 1H), 8.45 (s, 1H), 8.40 (s, 1H), 7.72 (s, 1H), 7.28-7.21 (m, 2H), 7.15-7.07 (m, 2H), 5.05 (quin, J = 7.6 Hz, 1H), 3.78 |
| | | | | (t, J = 6.7 Hz, 2H), 3.64-3.48 |
| | | | | (m, 2H), 3.20-3.05 (m, 1H), |
| | | | | 2.84-2.66 (m, 4H), 2.18 (br d, |
| | | | | J = 11.1 Hz, 2H), 1.96-1.90 |
| | | | | (m, 2H), 1.66 (d, J = 6.9 Hz, |
| | | | | 3H), 1.58-1.36 (m, 5H), 1.19 |
| | | | | (q, J = 11.9 Hz, 2H) |
| | | | | Six protons are not apparent |
| | | | | ¹⁹F-NMR (470 MHz, DMSO- |
| | | | | d₆): δ ppm −121.77 |
| P-371 | A-57 | C-139 | 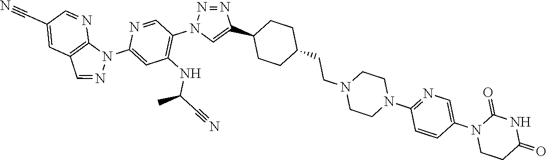 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)cn4)CC3)CC2)nn1] | 47% yield [M + H]⁺ = 740.3 ¹H-NMR (400 MHz, DMSO- d₆): δ ppm 1.14-1.22 (m, 2H), 1.36-1.58 (m, 3H), 1.62 (d, J = 7.2 Hz, 3H), 1.63- 1.69 (m, 2H), 1.88-1.90 (m, 2H), 2.15-2.18 (m, 2H), 2.72 (t, J = 6.8 Hz, 2H), 2.75-2.82 (m, 1H), 3.05-3.28 (m, 6H), |
| | | | | 3.61-3.64 (m, 2H), 3.73 (t, |
| | | | | J = 6.8 Hz, 2H), 4.39-4.42 |
| | | | | (m, 2H), 4.99-5.05 (m, 1H), |
| | | | | 7.02 (d, J = 10.2 Hz, 1H), |
| | | | | 7.20 (d, J = 8.0 Hz, 1H), 7.61- |
| | | | | 7.64 (m, 1H), 7.68 (s, 1H), |
| | | | | 8.15 (s, 1H), 8.37 (s, 1H), |
| | | | | 8.41 (s, 1H), 8.73 (s, 1H), |
| | | | | 9.06-9.08 (m, 2H), 10.39 (s, |
| | | | | 1H). |
| P-351 | A-57 | C-140 | 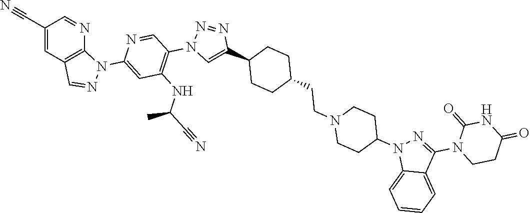 [SMILES: C[C@H](C#N)Nc1cc(-n2ncc3cc(C#N)cnc32)ncc1-n1cc([C@H]2CC[C@H](CCN3CCC(n4nc(N5CCC(=O)NC5=O)c5ccccc54)CC3)CC2)nn1] | 14% yield [M + H]⁺ = 778.3 ¹H-NMR not furnished |
| P-362 | A-57 | C-141 | 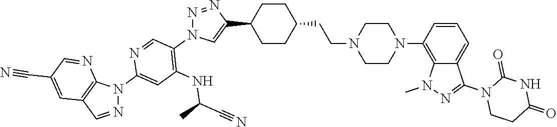 [SMILES: C[C@H](C#N)Nc1cc(-n2ncc3cc(C#N)cnc32)ncc1-n1cc([C@H]2CC[C@H](CCN3CCN(c4cccc5c(N6CCC(=O)NC6=O)nn(C)c45)CC3)CC2)nn1] | 8.8% yield [M + H]⁺ = 793.3 ¹H-NMR (500 MHz, DMSO- d₆) δ 10.56 (s, 1H), 9.06 (dd, J = 7.8, 1.9 Hz, 2H), 8.72 (s, 1H), 8.42 (s, 1H), 8.37 (s, 1H), 7.68 (s, 1H), 7.43 (d, J = 8.0 Hz, 1H), 7.20 (br d, |
| | | | | J = 8.0 Hz, 3H), 7.15-7.02 |
| | | | | (m, 3H), 5.02 (t, J = 7.2 Hz, |
| | | | | 1H), 4.26 (s, 2H), 3.92-3.88 |
| | | | | (m, 3H), 2.77 (t, J = 6.7 Hz, |
| | | | | 2H), 2.56-2.52 (m, 4H), 2.17 |
| | | | | (br d, J = 12.3 Hz, 2H), 1.90 |
| | | | | (br d, J = 9.5 Hz, 2H), 1.76 |
| | | | | (s, 3H), 1.62 (d, J = 6.9 Hz, |
| | | | | 5H), 1.57-1.39 (m, J = 12.0 |
| | | | | Hz, 3H), 1.29-1.14 (m, J = |
| | | | | 18.1 Hz, 3H) |
| P-330 | A-57 | C-142 | 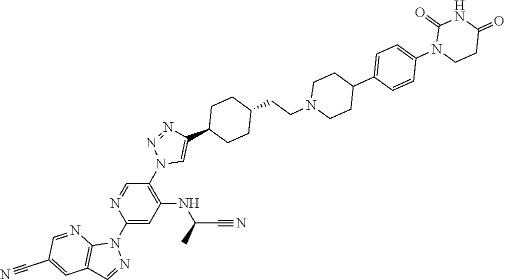 [SMILES: C[C@H](C#N)Nc1cc(-n2ncc3cc(C#N)cnc32)ncc1-n1cc([C@H]2CC[C@H](CCN3CCC(c4ccc(N5CCC(=O)NC5=O)cc4)CC3)CC2)nn1] | 18.8% yield [M + H]⁺ = 736.3 ¹H-NMR (400 MHz, DMSO- d₆): δ ppm 1.13-1.21 (m, 2H), 1.41-1.57 (m, 3H), 1.62 (d, J = 6.8 Hz, 3H), 1.67-1.70 (m, 2H), 1.88-1.91 (m, 2H), 2.18-2.20 (m, 2H), 2.66-2.80 (m, 4H), 3.22-3.29 (m, 3H), 3.64-3.78 (m, 3H), 4.99-5.03 (m, 1H), 6.23 (s, 1H), 7.18- 7.22 (m, 1H), 7.37 (d, J = 8.8 Hz, 2H), 7.54 (d, J = 8.4 Hz, 2H), 7.68 (s, 1H), 8.38 (s, 1H), 8.41 (s, 1H), 8.72-8.74 (m, 1H), 9.06-9.08 (m, 2H), 10.40 (s, 1H). Three protons are not apparent |
| P-370 | A-57 | C-143 | 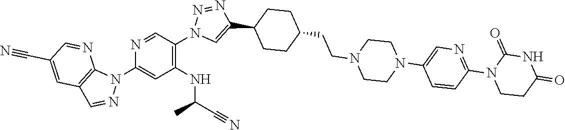 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)nc4)CC3)CC2)nn1] | 28.4% yield [M + H]⁺ = 740.3 ¹H-NMR (400 MHz, DMSO- d₆): δ ppm 1.15-1.26 (m, 2H), 1.38-1.58 (m, 3H), 1.62 (d, J = 7.2 Hz, 3H), 1.62-1.68 (m, 2H), 1.88-1.92 (m, 2H), 2.16-2.18 (m, 2H), 2.66-2.70 |
| | | | | (m, 2H), 2.74-2.81 (m, 1H), |
| | | | | 3.01-3.08 (m, 2H), 3.15-3.27 |
| | | | | (m, 4H), 3.62-3.65 (m, 2H), |
| | | | | 3.89-3.92 (m, 2H), 3.96 (t, |
| | | | | J = 6.8 Hz, 2H), 4.99-5.08 |
| | | | | (m, 1H), 7.19 (d, J = 8.4 Hz, |
| | | | | 1H), 7.52-7.61 (m, 2H), 7.68 |
| | | | | (s, 1H), 8.17 (d, J = 2.8 Hz, |
| | | | | 1H), 8.37 (s, 1H), 8.42 (s, |
| | | | | 1H), 8.73 (s, 1H), 9.06-9.08 |
| | | | | (m, 2H), 10.44 (s, 1H). |
| P-384 | A-104 | C-118 | 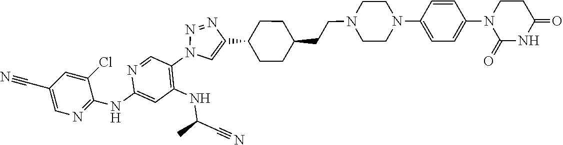 [SMILES: C[C@H](C#N)Nc1cc(Nc2ncc(C#N)cc2Cl)ncc1-n1cc([C@H]2CC[C@H](CCN3CCN(c4ccc(N5CCC(=O)NC5=O)cc4)CC3)CC2)nn1] | 29.6% yield [M + H]⁺ = 748.2 ¹H-NMR (400 MHz, DMSO- d₆): δ ppm 1.14-1.25 (m, 2H), 1.35-1.52 (m, 3H), 1.60 (d, J = 7.2 Hz, 3H), 1.62- 1.70 (m, 2H), 1.87-1.90 (m, 2H), 2.13-2.16 (m, 2H), 2.66-2.78 (m, 3H), 2.96-3.02 |
| | | | | (m, 2H), 3.15-3.28 (m, 4H), |
| | | | | 3.61-3.64 (m, 2H), 3.72 (t, |
| | | | | J = 6.8 Hz, 2H), 3.85-3.88 |
| | | | | (m, 2H), 4.78-4.86 (m, 1H), |
| | | | | 6.92 (d, J = 8.0 Hz, 1H), 7.04 |
| | | | | (d, J = 9.2 Hz, 2H), 7.23 (d, |
| | | | | J = 9.2 Hz, 2H), 7.75 (s, 1H), |
| | | | | 8.15 (s, 1H), 8.27 (s, 1H), |
| | | | | 8.48 (d, J = 2.0 Hz, 1H), 8.64 |
| | | | | (d, J = 2.0 Hz, 1H), 9.22 (s, |
| | | | | 1H), 10.29 (s, 1H). |
| P-334 | A-57 | C-144 | 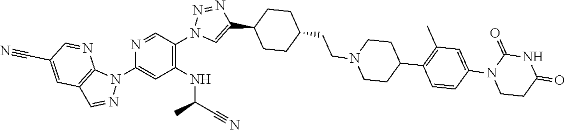 [SMILES: Cc1cc(N2CCC(=O)NC2=O)ccc1C1CCN(CC[C@H]2CC[C@H](c3cn(-c4cnc(-n5ncc6cc(C#N)cnc65)cc4N[C@H](C)C#N)nn3)CC2)CC1] | 33.7% yield [M + H]⁺ = 752.4 ¹H-NMR (400 MHz, DMSO- d₆): δ ppm 1.13-1.22 (m, 2H), 1.38-1.65 (m, 3H), 1.62 (d, J = 6.8 Hz, 3H), 1.65- 1.69 (m, 2H), 1.84-1.96 (m, 5H), 2.16-2.19 (m, 2H), 2.35 |
| | | | | (s, 3H), 2.68-2.72 (m, 2H), |
| | | | | 2.73-2.81 (m, 1H), 3.01-3.20 |
| | | | | (m, 5H), 3.61-3.64 (m, 2H), |
| | | | | 3.74-3.77 (m, 2H), 4.99-5.05 |
| | | | | (m, 1H), 7.16-7.21 (m, 4H), |
| | | | | 7.68 (s, 1H), 8.37-8.42 (m, |
| | | | | 2H), 8.73 (s, 1H), 9.06-9.08 |
| | | | | (m, 2H), 10.34 (s, 1H). |
| | | | | One proton is not apparent |
| P-341 | A-57 | C-145 | 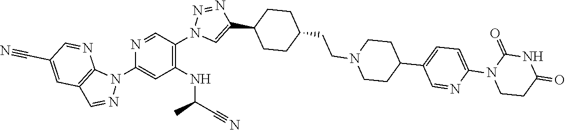 [SMILES: C[C@H](C#N)Nc1cc(-n2ncc3cc(C#N)cnc32)ncc1-n1cc([C@H]2CC[C@H](CCN3CCC(c4ccc(N5CCC(=O)NC5=O)nc4)CC3)CC2)nn1] | 12.9% yield [M + H]⁺ = 739.4 ¹H-NMR (400 MHz, DMSO- d₆): δ ppm 1.10-1.28 (m, 2H), 1.36-1.58 (m, 3H), 1.62 (d, J = 7.2 Hz, 3H), 1.63- 1.70 (m, 2H), 1.88-1.91 (m, 4H), 2.05-2.18 (m, 4H), 2.69 |
| | | | | (t, J = 6.8 Hz, 2H), 2.75-2.81 |
| | | | | (m, 1H), 2.88-2.94 (m, 1H), |
| | | | | 3.05-3.12 (m, 2H), 3.17-3.22 |
| | | | | (m, 2H), 3.62-3.66 (m, 2H), |
| | | | | 4.04 (t, J = 6.8 Hz, 2H), 4.98- |
| | | | | 5.06 (m, 1H), 7.21-7.25 (m, |
| | | | | 1H), 7.68-6.75 (m, 3H), 8.33 |
| | | | | (s, 1H), 8.37 (s, 1H), 8.42 (s, |
| | | | | 1H), 8.72 (s, 1H), 9.06-9.07 |
| | | | | (m, 2H), 10.52 (s, 1H). |
| P-336 | A-57 | C-146 | 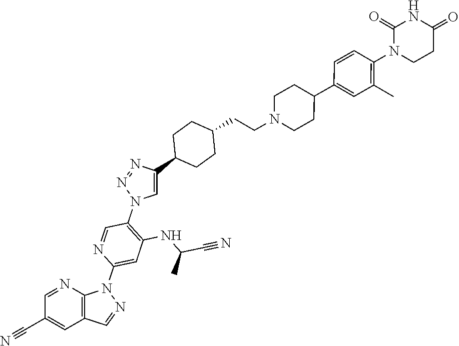 [SMILES: Cc1cc(C2CCN(CC[C@H]3CC[C@H](c4cn(-c5cnc(-n6ncc7cc(C#N)cnc76)cc5N[C@H](C)C#N)nn4)CC3)CC2)ccc1N1CCC(=O)NC1=O] | 35.7% yield [M + H]⁺ = 752.3 ¹H-NMR (400 MHz, DMSO- d₆): δ ppm 1.15-1.24 (m, 2H), 1.38-1.56 (m, 3H), 1.62 (d, J = 6.8 Hz, 3H), 1.65- 1.68 (m, 2H), 1.85-1.92 (m, 4H), 2.04-2.07 (m, 2H), 2.16-2.19 (m, 2H), 2.19 (s, 3H), 2.67-2.85 (m, 4H), 3.02- 3.11 (m, 2H), 3.17-3.20 (m, 2H), 3.50-3.54 (m, 1H), 3.60- 3.66 (m, 2H), 3.73-3.78 (m, 1H), 4.98-5.05 (m, 1H), 7.14- 7.28 (m, 4H), 7.68 (s, 1H), 8.37 (s, 1H), 8.42 (s, 1H), 8.73 (s, 1H), 9.06-9.09 (m, 2H), 10.33 (s, 1H). |
| P-373 | A-57 | C-118 | 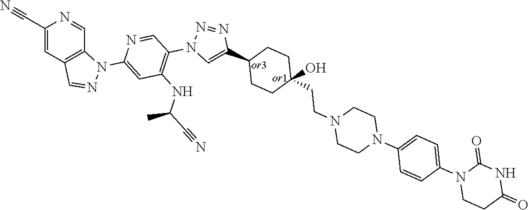 [SMILES: C[C@H](C#N)Nc1cc(-n2ncc3cc(C#N)ncc32)ncc1-n1cc([C@H]2CC[C@](O)(CCN3CCN(c4ccc(N5CCC(=O)NC5=O)cc4)CC3)CC2)nn1] | 5.1% yield [M + H]⁺ = 755.3 ¹H-NMR (400 MHz, DMSO- d₆): δ ppm 1.55-1.62 (m, 5H), 1.68-1.80 (m, 4H), 1.89- 1.93 (m, 2H), 2.07-2.10 (m, 2H), 2.69 (d, J = 6.8 Hz, 2H), 2.84-2.91 (m, 1H), 2.94-3.00 (m, 2H), 3.16-3.30 (m, 4H), 3.63-3.72 (m, 4H), 3.83-3.86 (m, 2H), 4.98-5.05 (m, 1H), 7.02 (d, J = 8.8 Hz, 2H), 7.19- 7.26 (m, 3H), 7.68 (s, 1H), |
| | | | | 8.37-8.42 (m, 2H), 8.72 (s, |
| | | | | 1H), 9.05-9.07 (m, 2H), 10.28 |
| | | | | (s, 1H). |
| | | | | One proton is not apparent |
| P-329 | A-57 | C-147 | 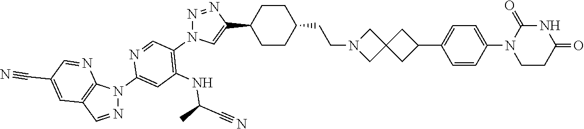 [SMILES: C[C@H](C#N)Nc1cc(-n2ncc3cc(C#N)cnc32)ncc1-n1cc([C@H]2CC[C@H](CCN3CC4(CC(c5ccc(N6CCC(=O)NC6=O)cc5)C4)C3)CC2)nn1] | 5.2% yield [M + H]⁺ = 750.2 ¹H-NMR (400 MHz, DMSO- d₆): δ ppm 1.09-1.18 (m, 2H), 1.36-1.52 (m, 5H), 1.61 (d, J = 6.8 Hz, 3H), 1.83- 1.86 (m, 2H), 2.13-2.16 (m, 2H), 2.27-2.40 (m, 2H), 2.52- |
| | | | | 2.59 (m, 1H), 2.67-2.87 (m, |
| | | | | 4H), 3.17-3.21 (m, 2H), 3.74- |
| | | | | 3.77 (m, 2H), 3.94-3.99 (m, |
| | | | | 1H), 4.06-4.15 (m, 2H), 4.31- |
| | | | | 4.35 (m, 1H), 4.98-5.05 (m, |
| | | | | 1H), 7.18-7.28 (m, 5H), 7.68 |
| | | | | (s, 1H), 8.36 (s, 1H), 8.41 (s, |
| | | | | 1H), 8.72 (s, 1H), 9.05-9.07 |
| | | | | (m, 2H), 10.33 (s, 1H). |
| | | | | One proton is not apparent |
| P-369 | A-57 | C-148 | 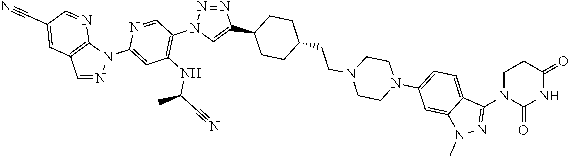 [SMILES: C[C@H](C#N)Nc1cc(-n2ncc3cc(C#N)cnc32)ncc1-n1cc([C@H]2CC[C@H](CCN3CCN(c4ccc5c(N6CCC(=O)NC6=O)nn(C)c5c4)CC3)CC2)nn1] | 12.3% yield [M + H]⁺ = 793.2 ¹H-NMR (500 MHz, DMSO- d₆) Shift 10.56 (s, 1H), 9.13- 9.08 (m, 2H), 8.76 (s, 1H), 8.45 (s, 1H), 8.41 (s, 1H), 7.72 (s, 1H), 7.58 (d, J = 9.5 Hz, 1H), 7.28 (s, 1H), 7.24 (br d, J = 8.0 Hz, 1H), 7.21- 7.17 (m, 1H), 7.08 (s, 1H), |
| | | | | 7.05-7.00 (m, 2H), 5.06 |
| | | | | (quin, J = 7.4 Hz, 1H), 4.03 |
| | | | | (br d, J = 13.9 Hz, 1H), 3.99- |
| | | | | 3.93 (m, 5H), 3.51 (br s, 3H), |
| | | | | 3.30-3.18 (m, 1H), 3.17-3.08 |
| | | | | (m, 1H), 2.84-2.76 (m, 3H), |
| | | | | 2.21 (br d, J = 12.6 Hz, 2H), |
| | | | | 1.94 (br d, J = 10.9 Hz, 2H), |
| | | | | 1.77-1.69 (m, 2H), 1.66 (d, |
| | | | | J = 6.8 Hz, 4H), 1.61-1.48 (m, |
| | | | | 3H), 1.48-1.37 (m, 1H), 1.29- |
| | | | | 1.12 (m, 3H) |
| P-386 | A-107 | C-118 | 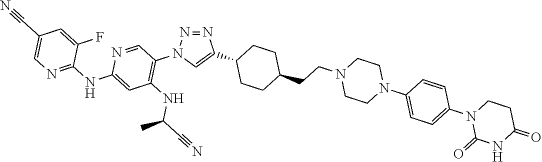 [SMILES: C[C@H](C#N)Nc1cc(Nc2ncc(C#N)cc2F)ncc1-n1cc([C@H]2CC[C@H](CCN3CCN(c4ccc(N5CCC(=O)NC5=O)cc4)CC3)CC2)nn1] | 22.6% yield [M + H]⁺ = 732.4 ¹H-NMR (400 MHz, DMSO- d₆): δ ppm 1.12-1.25 (m, 2H), 1.35-1.54 (m, 3H), 1.60 (d, J = 6.8 Hz, 3H), 1.62-1.68 (m, 2H), 1.86-1.89 (m, 2H), 2.12-2.15 (m, 2H), 2.66-2.75 (m, 3H), 2.95-3.02 (m, 2H), 3.14-3.26 (m, 4H), 3.60-3.63 (m, 2H), 3.71 (t, J = 6.8 Hz, 2H), 3.84-3.87 (m, 2H), 4.75- 4.85 (m, 1H), 6.87 (d, J = 8.0 Hz, 1H), 7.03 (d, J = 8.8 Hz, 2H), 7.22 (d, J = 8.8 Hz, 2H), |
| | | | | 7.71 (s, 1H), 8.13 (s, 1H), |
| | | | | 8.21-8.25 (m, 2H), 8.52 (d, |
| | | | | J = 2.0 Hz, 1H) 10.12 (s, 1H), |
| | | | | 10.29 (s, 1H). |
| | | | | ¹⁹F-NMR (376 MHz, DMSO- |
| | | | | d₆): δ ppm −130.91 |
| P-378 | A-104 | C-128 | 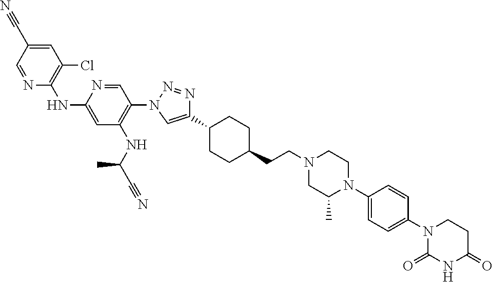 [SMILES: C[C@H](C#N)Nc1cc(Nc2ncc(C#N)cc2Cl)ncc1-n1cc([C@H]2CC[C@H](CCN3CCN(c4ccc(N5CCC(=O)NC5=O)cc4)[C@H](C)C3)CC2)nn1] | 6.1% yield [M + H]⁺ = 762.4 ¹H-NMR (400 MHz, DMSO- d₆): δ ppm 0.93-0.94 (m, 1H), 1.10-1.12 (m, 2H), 1.17- 1.24 (m, 2H), 1.35-1.41 (m, 1H), 1.43-1.52 (m, 2H), 1.61 (d, J = 6.8 Hz, 3H), 1.65-1.71 (m, 2H), 1.87-1.90 (m, 2H), 2.13-2.16 (m, 2H), 2.67-2.75 (m, 3H), 3.04-3.15 (m, 2H), 3.22-3.29 (m, 4H), 3.70-3.78 (m, 2H), 4.37 (brs, 1H), 4.79- 4.85 (m, 1H), 6.97-6.98 (m, 2H), 7.17-7.23 (m, 2H), 7.32 (d, J = 8.4 Hz, 1H), 7.74 (s, 1H), 8.16 (s, 1H), 8.27 (s, 1H), 8.48 (d, J = 2.0 Hz, 1H), |
| | | | | 8.64 (d, J = 2.0 Hz, 1H), 9.28 |
| | | | | (s, 1H), 10.28 & 10.36 (s, |
| | | | | 1H). |
| | | | | Two protons are not apparent |
| P-393 | A-57 | C-149 | 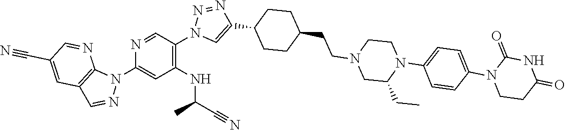 [SMILES: CC[C@@H]1CN(CC[C@H]2CC[C@H](c3cn(-c4cnc(-n5ncc6cc(C#N)cnc65)cc4N[C@H](C)C#N)nn3)CC2)CCN1c1ccc(N2CCC(=O)NC2=O)cc1] | 17.6% yield [M + H]⁺ = 767.3 ¹H-NMR (500 MHz, DMSO- d₆) Shift 10.29 (s, 1H), 9.11 (s, 1H), 9.10 (br d, J = 1.8 Hz, 1H), 8.76 (s, 1H), 8.45 (s, 1H), 8.40 (s, 1H), 7.72 (s, 1H), 7.26-7.20 (m, 1H), 7.20- |
| | | | | 7.14 (m, 2H), 6.99-6.82 (m, |
| | | | | 2H), 5.06 (quin, J = 4.7 Hz, |
| | | | | 1H), 3.95 (s, 1H), 3.74 (t, J = |
| | | | | 6.3 Hz, 2H), 2.82-2.76 (m, |
| | | | | 1H), 2.73 (t, J = 6.8 Hz, 2H), |
| | | | | 2.19 (br d, J = 12.9 Hz, 2H), |
| | | | | 1.95 (br d, J = 10.9 Hz, 2H), |
| | | | | 1.88-1.76 (m, 1H), 1.66 (d, |
| | | | | J = 7.0 Hz, 4H), 1.59-1.42 |
| | | | | (m, 5H), 1.39-1.13 (m, 3H), |
| | | | | 0.88 (br t, J = 6.7 Hz, 3H) |
| | | | | Seven protons are not |
| | | | | apparent |
| P-385 | A-107 | C-128 | 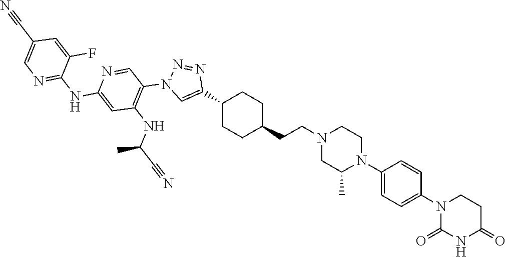 [SMILES: C[C@H](C#N)Nc1cc(Nc2ncc(C#N)cc2F)ncc1-n1cc([C@H]2CC[C@H](CCN3CCN(c4ccc(N5CCC(=O)NC5=O)cc4)[C@H](C)C3)CC2)nn1] | 27.9% yield [M + H]⁺ = 746.2 ¹H-NMR (400 MHz, DMSO- d₆, 100 oC): δ ppm 1.10 (brs, 3H), 1.21-1.28 (m, 2H), 1.42- 1.60 (m, 3H), 1.65 (d, J = 7.2 Hz, 3H), 1.66-1.78 (m, 2H), 1.90-1.94 (m, 2H), 2.16-2.20 (m, 2H), 2.71-2.84 (m, 3H), 3.20-3.32 (m, 4H), 3.67-3.77 (m, 3H), 4.73-4.76 (m, 1H), 6.67 (d, J = 7.6 Hz, 1H), 7.05 (brs, 2H), 7.27-7.29 (m, 2H), 7.78 (s, 1H), 8.09-8.23 (m, 3H), 8.49 (s, 1H), 9.56 (brs, 1H), 9.88 (s, 1H). Four protons are not apparent ¹⁹F-NMR (376 MHz, DMSO- d₆): δ ppm −130.885 |
| P-359 | A-57 | C-150 | 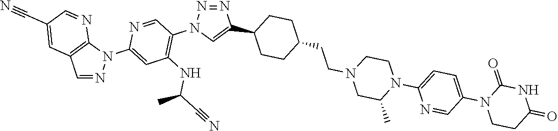 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)cn4)[C@H](C)C3)CC2)nn1] | 2.5% yield [M + H]⁺ = 754.4 ¹H-NMR not furnished |
| P-372 | A-57 | C-151 | 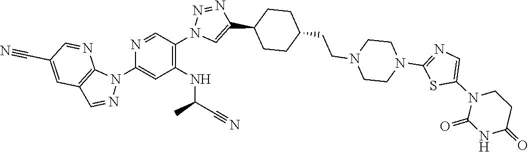 [SMILES: C[C@H](C#N)Nc1cc(-n2ncc3cc(C#N)cnc32)ncc1-n1cc([C@H]2CC[C@H](CCN3CCN(c4ncc(N5CCC(=O)NC5=O)s4)CC3)CC2)nn1] | 0.7% yield [M + H]⁺ = 746.4 ¹H-NMR not furnished |
| P-361 | A-57 | C-152 | 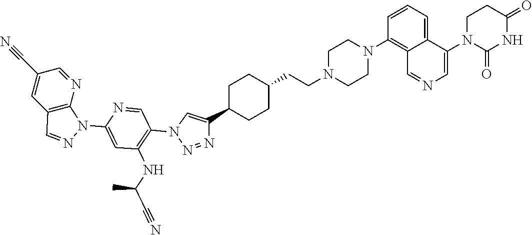 [SMILES: C[C@H](C#N)Nc1cc(-n2ncc3cc(C#N)cnc32)ncc1-n1cc([C@H]2CC[C@H](CCN3CCN(c4cccc5c(N6CCC(=O)NC6=O)cncc45)CC3)CC2)nn1] | 12.3% yield [M + H]⁺ = 790.3 ¹H-NMR (500 MHz, DMSO- d₆) Shift 10.60 (s, 1H), 9.58 (s, 1H), 9.13-9.08 (m, 2H), 8.77 (s, 1H), 8.65 (s, 1H), 8.46 (s, 1H), 8.42 (s, 1H), 7.87-7.76 (m, 2H), 7.73 (s, 1H), 7.44 (d, J = 7.5 Hz, 1H), 7.25 (d, J = 7.7 Hz, 1H), 5.07 (quin, J = 7.4 Hz, 1H), 4.01- 3.93 (m, 1H), 3.79-3.57 (m, 3H), 3.43-3.20 (m, 2H), 3.08- 2.99 (m, 1H), 2.87-2.77 (m, 2H), 2.23 (br d, J = 13.3 Hz, |
| | | | | 2H), 1.97 (br d, J = 12.6 Hz, |
| | | | | 2H), 1.75 (br s, 2H), 1.66 (d, |
| | | | | J = 6.9 Hz, 3H), 1.62-1.45 |
| | | | | (m, 3H), 1.31-1.18 (m, 3H) |
| | | | | Five protons are not apparent |
| P-389 | A-108 | C-118 | 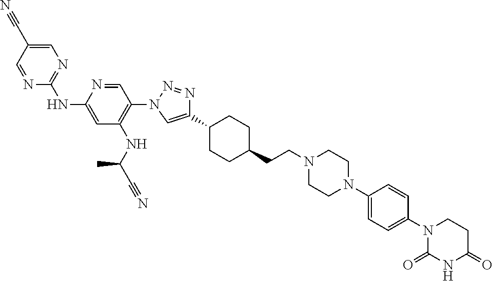 [SMILES: C[C@H](C#N)Nc1cc(Nc2ncc(C#N)cn2)ncc1-n1cc([C@H]2CC[C@H](CCN3CCN(c4ccc(N5CCC(=O)NC5=O)cc4)CC3)CC2)nn1] | 33.9% yield [M + H]⁺ = 715.4 ¹H-NMR (400 MHz, DMSO- d₆): δ ppm 1.08-1.21 (m, 2H), 1.30-1.58 (m, 3H), 1.60 (d, J = 6.8 Hz, 3H), 1.65-1.70 (m, 2H), 1.86-1.90 (m, 2H), 2.12-2.15 (m, 2H), 2.68-2.83 (m, 3H), 2.98-3.02 (m, 2H), 3.12-3.28 (m, 4H), 3.69-3.73 (m, 2H), 3.84-3.87 (m, 2H), 4.75-4.82 (m, 1H), 6.88 (d, J = 8.0 Hz, 1H), 7.03 (d, J = 9.2 Hz, 2H), 7.22 (d, J = 8.8 Hz, 2H), 7.89 (s, 1H), 8.14 (s, 1H), 8.25 (s, 1H), 8.99 (s, 2H), 10.29 (s, 1H), 10.91 (s, |
| | | | | 1H). |
| | | | | Two protons are not apparent |
| P-357 | A-57 | C-153 | 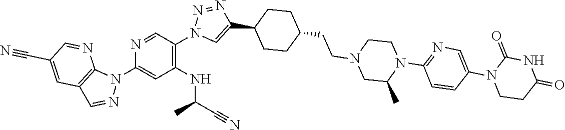 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)cn4)[C@@H](C)C3)CC2)nn1] | 14.4% yield [M + H]⁺ = 754.2 ¹H-NMR not furnished |
| P-394 | A-57 | C-154 | 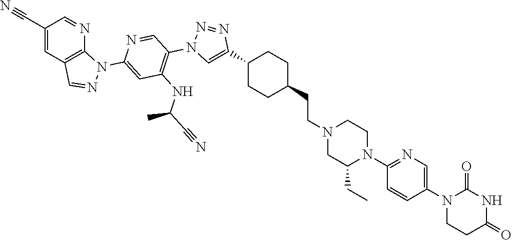 [SMILES: CC[C@@H]1CN(CC[C@H]2CC[C@H](c3cn(-c4cnc(-n5ncc6cc(C#N)cnc65)cc4N[C@H](C)C#N)nn3)CC2)CCN1c1ccc(N2CCC(=O)NC2=O)cn1] | 37% yield [M + H]⁺ = 768.3 ¹H-NMR (500 MHz, DMSO- d₆) δ 10.56 (s, 1H), 9.53 (s, 1H), 9.07 (br d, J = 6.2 Hz, 2H), 8.72 (s, 1H), 8.61 (s, 1H), 8.42 (s, 1H), 8.38 (s, 1H), 7.69 (s, 1H), 7.40 (d, J = 7.3 Hz, 1H), 7.26-7.18 (m, 2H), 6.86-6.78 (m, 1H), 5.03 (br t, J = 7.5 Hz, 1H), 3.97- 3.89 (m, 1H), 3.75-3.53 (m, 3H), 3.17 (s, 1H), 2.98 (br d, J = 9.9 Hz, 1H), 2.82-2.73 (m, 2H), 2.55 (s, 5H), 2.18 |
| | | | | (br d, J = 13.2 Hz, 2H), 1.93 |
| | | | | (br d, J = 12.6 Hz, 2H), 1.70 |
| | | | | (br s, 2H), 1.62 (d, J = 6.9 |
| | | | | Hz, 3H), 1.58-1.40 (m, 3H), |
| | | | | 1.27-1.14 (m, 3H) |
| P-399 | A-57 | C-155a | 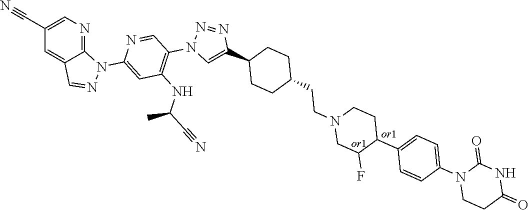 [SMILES: C[C@H](C#N)Nc1cc(-n2ncc3cc(C#N)cnc32)ncc1-n1cc([C@H]2CC[C@H](CCN3CCC(c4ccc(N5CCC(=O)NC5=O)cc4)C(F)C3)CC2)nn1] | 25.8% yield [M + H]⁺ = 757.4 ¹H-NMR (400 MHz, DMSO- d₆): δ ppm 1.17-1.24 (m, 2H), 1.42-1.53 (m, 3H), 1.61 (d, J = 6.8 Hz, 3H), 1.68-1.70 (m, 2H), 1.88-1.91 (m, 3H), 2.10-2.19 (m, 3H), 2.66-2.82 (m, 3H), 3.04-3.28 (m, 5H), 3.79 (t, J = 6.8 Hz, 2H), 3.90- 3.98 (m, 1H), 4.85-5.04 (m, 2H), 7.18-7.21 (m, 1H), 7.30- 7.36 (m, 4H), 7.68 (s, 1H), |
| | | | | 8.37 (s, 1H), 8.41 (s, 1H), |
| | | | | 8.72 (s, 1H), 9.06-9.07 (m, |
| | | | | 2H), 10.38 (s, 1H). |
| | | | | One proton is not apparent |
| | | | | ¹⁹F-NMR (376 MHz, DMSO- |
| | | | | d₆): δ ppm −184.365 |
| P-400 | A-57 | C-155b | 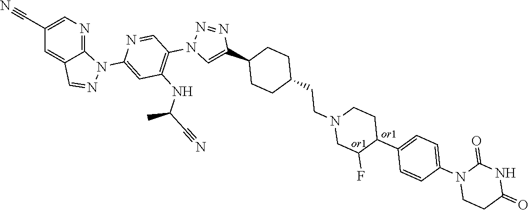 [SMILES: C[C@H](C#N)Nc1cc(-n2ncc3cc(C#N)cnc32)ncc1-n1cc([C@H]2CC[C@H](CCN3CCC(c4ccc(N5CCC(=O)NC5=O)cc4)C(F)C3)CC2)nn1] | 26.2% yield [M + H]⁺ = 756.4 ¹H-NMR (400 MHz, DMSO- d₆): δ ppm 1.12-1.28 (m, 2H), 1.38-1.53 (m, 3H), 1.61 (d, J = 6.8 Hz, 3H), 1.68- 1.70 (m, 2H), 1.89-1.91 (m, 3H), 2.10-2.18 (m, 3H), 2.66-2.81 (m, 3H), 3.02-3.30 (m, 5H), 3.79 (t, J = 6.8 Hz, 2H), 3.90-3.96 (m, 1H), 4.80- 5.04 (m, 2H), 7.18-7.21 (m, 1H), 7.30-7.45 (m, 4H), 7.68 |
| | | | | (s, 1H), 8.37 (s, 1H), 8.41 (s, |
| | | | | 1H), 8.72 (s, 1H), 9.06-9.07 |
| | | | | (m, 2H), 10.38 (s, 1H). |
| | | | | one proton is not apparent |
| | | | | ¹⁹F-NMR (376 MHz, DMSO- |
| | | | | d₆): δ ppm −184.292 (s) |
| P-387 | A-108 | C-128 | 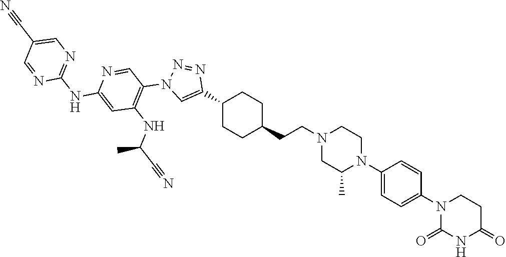 [SMILES: C[C@H](C#N)Nc1cc(Nc2ncc(C#N)cn2)ncc1-n1cc([C@H]2CC[C@H](CCN3CCN(c4ccc(N5CCC(=O)NC5=O)cc4)[C@H](C)C3)CC2)nn1] | 17.8% yield [M + H]⁺ = 728.7 ¹H-NMR (400 MHz, DMSO- d₆): δ ppm 0.93-1.12 (m, 3H), 1.12-1.38 (m, 2H), 1.40- 1.51 (m, 3H), 1.61 (d, J = 7.2 Hz, 3H), 1.63-1.70 (m, 2H), 1.87-1.90 (m, 2H), 2.13-2.16 (m, 2H), 2.67-2.73 (m, 3H), 3.01-3.30 (m, 6H), 3.60-3.80 (m, 4H), 4.35-4.40 (m, 1H), 4.78-4.80 (m, 1H), 6.90-7.00 (m, 2H), 7.17-7.33 (m, 3H), 7.87 (s, 1H), 8.15 (s, 1H), 8.26 (s, 1H), 9.00 (s, 2H), 10.28 and 10.36 (s, 1H), 10.96 (s, 1H). |
| P-401 | A-57 | C-156a | 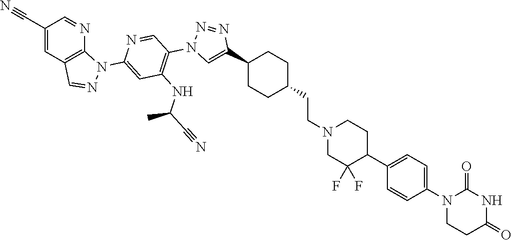 [SMILES: C[C@H](C#N)Nc1cc(-n2ncc3cc(C#N)cnc32)ncc1-n1cc([C@H]2CC[C@H](CCN3CCC(c4ccc(N5CCC(=O)NC5=O)cc4)C(F)(F)C3)CC2)nn1] | 46.4% yield [M + H]⁺ = 773.7 ¹H-NMR (400 MHz, DMSO- d₆): δ ppm 1.10-1.28 (m, 2H), 1.35-1.53 (m, 3H), 1.61 (d, J = 6.8 Hz, 3H), 1.68-1.80 (m, 2H), 1.87-1.91 (m, 2H), 2.15- 2.32 (m, 3H), 2.67-2.77 (m, 3H), 3.10-3.30 (m, 4H), 3.81 (t, J = 6.8 Hz, 2H), 4.03-4.11 (m, 1H), 4.98-5.08 (m, 1H), 7.18-7.20 (m, 1H), 7.32-7.37 (m, 4H), 7.68 (s, 1H), 8.37 (s, 1H), 8.41-8.43 (m, 1H), 8.72 (s, 1H), 9.06-9.07 (m, 2H), |
| | | | | 10.39 (s, 1H). |
| | | | | Three protons are not |
| | | | | apparent |
| | | | | ¹⁹F-NMR (376 MHz, DMSO- |
| | | | | d₆): δ ppm −101.686 (d, 2JF = |
| | | | | 251.9 Hz), −109.224 (d, 2JF = |
| | | | | 253.1 Hz) |
| P-396 | A-57 | C-158 | 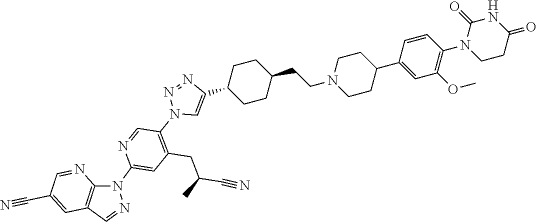 [SMILES: COc1cc(C2CCN(CC[C@H]3CC[C@H](c4cn(-c5cnc(-n6ncc7cc(C#N)cnc76)cc5C[C@H](C)C#N)nn4)CC3)CC2)ccc1N1CCC(=O)NC1=O] | 12.9% yield [M + H]⁺ = 768.2 ¹H-NMR (400 MHz, DMSO d6): δ ppm 1.11-1.21 (m, 3H), 1.39-1.52 (m, 4H), 1.61 (d, J = 6.8 Hz, 3H), 1.65- 1.67 (m, 2H), 1.88-1.91 (m, 4H), 2.08-2.20 (m, 4H), 2.72- 2.90 (m, 2H), 3.00-3.09 (m, 2H), 3.17-3.21 (m, 2H), 3.54- 3.57 (m, 2H), 3.61-3.64 (m, 2H), 3.82 (s, 3H), 4.99-5.03 (m, 1H), 6.86 (m, 1H), 6.95- 6.96 (m, 1H), 7.18-7.23 (m, |
| | | | | 2H), 7.68 (s, 1H), 8.37 (s, |
| | | | | 1H), 8.41 (s, 1H), 8.72 (s, |
| | | | | 1H), 9.06-9.07 (m, 2H), |
| | | | | 10.29 (s, 1H). |
| P-420 | A-57 | C-156b | 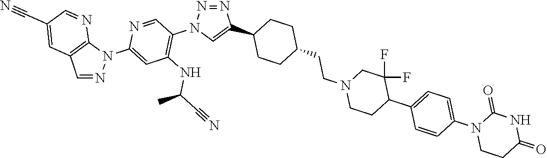 [SMILES: C[C@H](C#N)Nc1cc(-n2ncc3cc(C#N)cnc32)ncc1-n1cc([C@H]2CC[C@H](CCN3CCC(c4ccc(N5CCC(=O)NC5=O)cc4)C(F)(F)C3)CC2)nn1] | 16.6% yield [M + H]⁺ = 774.2 ¹H-NMR (400 MHz, DMSO- d₆): δ ppm 1.14-1.22 (m, 2H), 1.40-1.52 (m, 4H), 1.61 (d, J = 7.2 Hz, 3H), 1.63-1.70 (m, 2H), 1.88-1.91 (m, 2H), 2.15-2.18 (m, 3H), 2.66-2.83 (m, 3H), 3.79-3.82 (m, 2H), 4.97-5.05 (m, 1H), 7.19 (d, |
| | | | | J = 8.8 Hz, 1H), 7.32-7.37 |
| | | | | (m, 4H), 7.68 (s, 1H), 8.37 (s, |
| | | | | 1H), 8.41 (s, 1H), 8.73 (s, |
| | | | | 1H), 9.06-9.07 (m, 2H), |
| | | | | 10.39 (s, 1H). |
| | | | | Seven protons are not |
| | | | | apparent |
| | | | | ¹⁹F-NMR (376 MHz, DMSO- |
| | | | | d₆): δ ppm −101.678 (d, 2JF = |
| | | | | 258.7 Hz), −109.117 (d, 2JF = |
| | | | | 251.9 Hz) |
| P-421 | A-66 | C-128 | 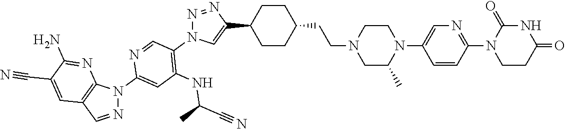 [SMILES: C[C@H](C#N)Nc1cc(-n2ncc3cc(C#N)c(N)nc32)ncc1-n1cc([C@H]2CC[C@H](CCN3CCN(c4ccc(N5CCC(=O)NC5=O)nc4)[C@H](C)C3)CC2)nn1] | 41.9% yield [M + H]⁺ = 768.3 ¹H-NMR (400 MHz, DMSO- d₆, 90 oC): δ ppm 1.08-1.09 (m, 3H), 1.21-1.28 (m, 2H), 1.45-1.57 (m, 3H), 1.67 (d, J = 6.8 Hz, 3H), 1.71-1.75 (m, 2H), 1.91-1.93 (m, 2H), |
| | | | | 2.16-2.21 (m, 2H), 2.72 (t, |
| | | | | J = 6.8 Hz, 2H) 2.77-2.83 (m, |
| | | | | 1H), 3.41-3.45 (m, 3H), 3.75 |
| | | | | (t, J = 6.8 Hz, 2H), 4.95-4.98 |
| | | | | (m, 1H), 6.94-7.07 (m, 5H), |
| | | | | 7.26-7.29 (m, 2H), 7.96 (s, |
| | | | | 1H), 8.23 (s, 1H), 8.28 (s, |
| | | | | 1H), 8.38 (s, 1H), 8.52 (s, |
| | | | | 1H), 9.94 (s, 1H). |
| | | | | Six protons are not apparent |
| P-422 | A-57 | C-160 | 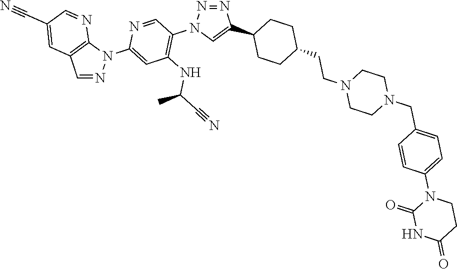 [SMILES: C[C@H](C#N)Nc1cc(-n2ncc3cc(C#N)cnc32)ncc1-n1cc([C@H]2CC[C@H](CCN3CCN(Cc4ccc(N5CCC(=O)NC5=O)cc4)CC3)CC2)nn1] | 25.2% yield [M + H]⁺ = 753.4 ¹H-NMR (500 MHz, DMSO- d₆) Shift 10.43 (s, 1H), 9.15- 9.09 (m, 2H), 8.77 (s, 1H), 8.45 (br s, 1H), 8.40 (s, 1H), 7.72 (br s, 1H), 7.40 (q, J = 8.4 Hz, 4H), 7.24 (br d, J = 8.1 Hz, 1H), 5.06 (br t, J = 7.2 Hz, 1H), 3.95 (s, 1H), 3.95 (s, 1H), 3.84 (t, J = 6.7 Hz, 2H), 3.27-3.04 (m, 4H), 2.83-2.72 (m, 3H), 2.66-2.58 (m, 1H), 2.19 (br d, J = 10.8 Hz, 2H), 2.12 (s, 1H), 1.90 (br d, J = 11.8 Hz, 2H), 1.70- 1.60 (m, J = 7.0 Hz, 5H), 1.52 |
| | | | | (q, J = 11.7 Hz, 2H), 1.42 |
| | | | | (br s, 1H), 1.21 (q, J = 12.5 |
| | | | | Hz, 2H) |
| | | | | Four protons are not apparent |
| P-423 | A-57 | C-161 | 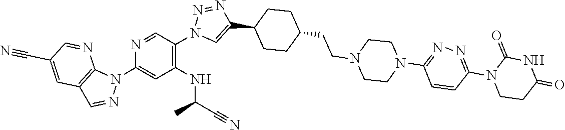 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)nn4)CC3)CC2)nn1] | 5.7% yield [M + H]⁺ = 741.5 ¹H-NMR (500 MHz, DMSO- d₆) Shift 10.65 (s, 1H), 9.14- 9.09 (m, 2H), 8.77 (s, 1H), 8.46 (br s, 1H), 8.41 (s, 1H), 7.80 (d, J = 9.7 Hz, 1H), 7.72 (s, 1H), 7.53 (d, J = 9.8 Hz, |
| | | | | 1H), 7.24 (br d, J = 8.0 Hz, |
| | | | | 1H), 5.07 (quin, J = 7.5 Hz, |
| | | | | 1H), 4.54-4.42 (m, 1H), 4.09 |
| | | | | (t, J = 6.7 Hz, 2H), 3.68 (br s, |
| | | | | 1H), 3.33-3.13 (m, 1H), 2.85- |
| | | | | 2.75 (m, 3H), 2.20 (br d, J = |
| | | | | 12.9 Hz, 2H), 1.93 (br d, J = |
| | | | | 10.4 Hz, 2H), 1.74-1.63 (m, |
| | | | | 5H), 1.55 (g, J = 11.9 Hz, |
| | | | | 2H), 1.45 (br s, 1H), 1.24 (q, |
| | | | | J = 11.3 Hz, 2H) |
| | | | | Seven protons are not |
| | | | | apparent |
| P-428 | A-66 | C-139 | 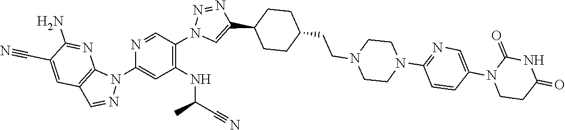 [SMILES: C[C@H](C#N)Nc1cc(-n2ncc3cc(C#N)c(N)nc32)ncc1-n1cc([C@H]2CC[C@H](CCN3CCN(c4ccc(N5CCC(=O)NC5=O)cn4)CC3)CC2)nn1] | 66.4% yield [M + H]⁺ = 755.4 ¹H-NMR (400 MHz, DMSO d6): δ ppm 1.12-1.21 (m, 2H), 1.37-1.54 (m, 3H), 1.63 (d, J = 6.8 Hz, 3H), 1.67- 1.70 (m, 2H), 1.87-1.90 (m, 2H), 2.15-2.17 (m, 2H), 2.71-2.79 (m, 3H), 3.03-3.25 |
| | | | | (m, 6H), 3.61-3.64 (m, 2H), |
| | | | | 3.71-3.75 (m, 2H), 4.39-4.42 |
| | | | | (m, 2H), 4.98-5.03 (m, 1H), |
| | | | | 7.02 (d, J = 9.2 Hz, 1H), 7.09 |
| | | | | (d, J = 8.4 Hz, 1H), 7.36 (brs, |
| | | | | 2H), 7.63 (dd, J = 2.8 Hz and |
| | | | | 9.2 Hz, 1H), 8.00 (s, 1H), |
| | | | | 8.16 (d, J = 2.8 Hz, 1H), 8.25 |
| | | | | (s, 1H), 8.33 (s, 1H), 8.34 (s, |
| | | | | 1H), 8.59 (s, 1H), 10.39 (s, |
| | | | | 1H). |
| P-429 | A-57 | C-118 | 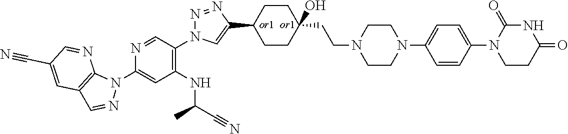 [SMILES: C[C@H](C#N)Nc1cc(-n2ncc3cc(C#N)cnc32)ncc1-n1cc([C@H]2CC[C@](O)(CCN3CCN(c4ccc(N5CCC(=O)NC5=O)cc4)CC3)CC2)nn1] | 23.7% yield [M + H]⁺ = 755.4 ¹H-NMR (400 MHz, DMSO- d₆): δ ppm 1.40-1.58 (m, 2H), 1.62 (d, J = 6.8 Hz, 3H), 1.73-1.76 (m, 2H), 1.83-1.91 (m, 6H), 2.65-2.70 (m, 2H), 2.75-2.82 (m, 1H), 2.95-3.02 (m, 2H), 3.16-3.25 (m, 2H), |
| | | | | 3.62-3.65 (m, 2H), 3.71 (t, |
| | | | | J = 6.8 Hz, 2H), 3.84-3.88 |
| | | | | (m, 2H), 4.45-4.55 (m, 1H), |
| | | | | 4.98-5.05 (m, 1H), 7.03 (d, |
| | | | | J = 9.2 Hz, 2H), 7.19-7.24 |
| | | | | (m, 3H), 7.68 (s, 1H), 8.37 (s, |
| | | | | 1H), 8.42 (s, 1H), 8.72 (s, |
| | | | | 1H), 9.05-9.07 (m, 2H), 10.29 |
| | | | | (s, 1H). |
| | | | | Two protons are not apparent |
| P-432 | A-57 | C-162 | 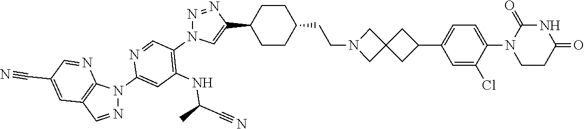 [SMILES: C[C@H](C#N)Nc1cc(-n2ncc3cc(C#N)cnc32)ncc1-n1cc([C@H]2CC[C@H](CCN3CC4(CC(c5ccc(N6CCC(=O)NC6=O)c(Cl)c5)C4)C3)CC2)nn1] | 13.4% yield [M + H]⁺ = 784.2 ¹H-NMR (400 MHz, DMSO- d₆): δ ppm 1.12-1.24 (m, 2H), 1.38-1.52 (m, 5H), 1.61 (d, J = 7.2 Hz, 3H), 1.83-1.86 (m, 2H), 2.13-2.16 (m, 2H), 2.34-2.49 (m, 2H), 2.54-2.60 (m, 1H), 2.65-2.80 (m, 4H), |
| | | | | 3.19-3.21 (m, 2H), 3.56-3.70 |
| | | | | (m, 2H), 3.92-3.98 (m, 1H), |
| | | | | 4.08-4.18 (m, 2H), 4.30-4.38 |
| | | | | (m, 1H), 4.98-5.08 (m, 1H), |
| | | | | 7.20-7.28 (m, 2H), 7.40-7.46 |
| | | | | (m, 2H), 7.68 (s, 1H), 8.36 (s, |
| | | | | 1H), 8.41 (s, 1H), 8.72 (s, |
| | | | | 1H), 9.05-9.07 (m, 2H), 10.44 |
| | | | | (s, 1H). |
| | | | | One protons is not apparent |
| P-434 | A-104 | C-147 | 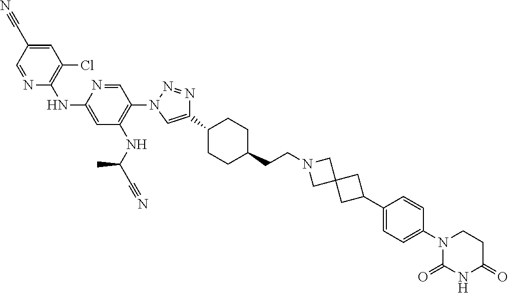 [SMILES: C[C@H](C#N)Nc1cc(Nc2ncc(C#N)cc2Cl)ncc1-n1cc([C@H]2CC[C@H](CCN3CC4(CC(c5ccc(N6CCC(=O)NC6=O)cc5)C4)C3)CC2)nn1] | 44.5% yield [M + H]⁺ = 759.3 ¹H-NMR (400 MHz, DMSO d6): δ ppm 1.08-1.17 (m, 2H), 1.38-1.51 (m, 5H), 1.61 (d, J = 6.8 Hz, 3H), 1.82-1.85 (m, 2H), 2.11-2.14 (m, 2H), 2.27- 2.39 (m, 2H), 2.55-2.60 (m, 1H), 2.66-2.75 (m, 4H), 3.17- 3.20 (m, 2H), 3.37-3.45 (m, 1H), 3.95-3.99 (m, 1H), 4.08- 4.16 (m, 2H), 4.33-4.36 (m, 1H), 4.82-4.88 (m, 1H), 7.19- 7.23 (m, 1H), 7.25-7.28 (m, 4H), 7.71 (s, 1H), 8.21 (s, 1H), 8.26 (s, 1H), 8.51 (d, J = 2.0 Hz, 1H), 8.66 (d, J = 1.6 Hz, 1H), 9.87-9.90 (m, 1H), |
| | | | | 10.33 (s, 1H). |
| | | | | Two protons are not apparent |
| P-436 | A-57 | C-163 | 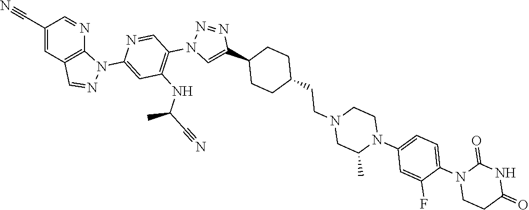 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)c(F)c4)[C@H](C)C3)CC2)nn1] | 28.5% yield [M + H]⁺ = 771.4 ¹H-NMR (400 MHz, DMSO- d₆, 100 oC): δ ppm 1.16-1.23 (m, 5H), 1.45-1.56 (m, 3H), 1.64 (d, J = 7.2 Hz, 3H), 1.65- 1.67 (m, 2H), 1.89-1.92 (m, 2H), 2.16-2.19 (m, 2H), 2.69 (t, J = 6.8 Hz, 2H), 2.72-2.79 (m, 1H), 3.14-3.30 (m, 5H), 3.64 (t, J = 6.8 Hz, 2H), 4.92- 4.96 (m, 1H), 6.82-6.84 (m, 2H), 7.03 (d, J = 8.0 Hz, 1H), |
| | | | | 7.24-7.29 (m, 1H), 7.73 (s, |
| | | | | 1H), 8.27 (s, 1H), 8.45 (brs, |
| | | | | 1H), 8.61 (brs, 1H), 8.94-8.98 |
| | | | | (m, 2H), 9.97 (s, 1H). |
| | | | | Four protons are not apparent |
| | | | | ¹⁹F-NMR (376 MHz, DMSO- |
| | | | | d₆): δ ppm −119.713 |
| P-437 | A-57 | C-164 | 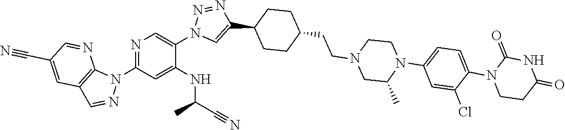 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)c(Cl)c4)[C@H](C)C3)CC2)nn1] | 10.2% yield [M + H]⁺ = 787.3 ¹H-NMR (400 MHz, DMSO d6): δ ppm 0.99-1.24 (m, 5H), 1.33-1.42 (m, 1H), 1.46-1.55 (m, 2H), 1.61 (d, J = 8.0 Hz, 3H), 1.63-1.70 (m, 2H), 1.87- 1.90 (m, 2H), 2.15-2.18 (m, |
| | | | | 2H), 2.69-2.79 (m, 3H), 3.02- |
| | | | | 3.10 (m, 1H), 3.53-3.64 (m, |
| | | | | 4H), 3.72-3.77 (m, 1H), 4.41- |
| | | | | 4.48 (m, 1H), 4.98-5.03 (m, |
| | | | | 1H), 6.99 (d, J = 4.0 Hz, 1H), |
| | | | | 7.11 (d, J = 2.8 Hz, 1H), 7.19 |
| | | | | (d, J = 8.4 Hz, 1H), 7.33 (d, |
| | | | | J = 8.4 Hz, 1H), 7.68 (s, 1H), |
| | | | | 8.37 (s, 1H), 8.41 (s, 1H), |
| | | | | 8.72 (s, 1H), 9.06-9.07 (m, |
| | | | | 2H), 10.38 and 10.45 (s, 1H). |
| | | | | Four protons are not apparent |
| P-438 | A-57 | C-165 | 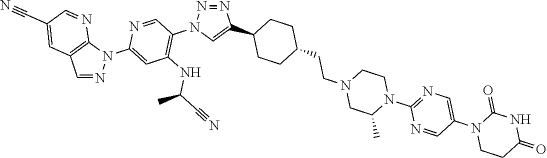 [SMILES: C[C@H](C#N)Nc1cc(-n2ncc3cc(C#N)cnc32)ncc1-n1cc([C@H]2CC[C@H](CCN3CCN(c4ncc(N5CCC(=O)NC5=O)cn4)[C@H](C)C3)CC2)nn1] | 16.3% yield [M + H]⁺ = 755.4 ¹H-NMR (400 MHz, DMSO d6): δ ppm 1.12-1.21 (m, 2H), 1.31 (d, J = 7.2 Hz, 3H), 1.37- 1.40 (m, 1H), 1.45-1.55 (m, 2H), 1.61 (d, J = 7.2 Hz, 3H), 1.66-1.70 (m, 2H), 1.87-1.90 (m, 2H), 2.15-2.18 (m, 2H), 2.71-2.77 (m, 3H), 2.98-3.05 |
| | | | | (m, 1H), 3.17-3.32 (m, 5H), |
| | | | | 3.71-3.74 (m, 3H), 4.70-4.74 |
| | | | | (m, 1H), 4.98-5.02 (m, 1H), |
| | | | | 5.05-5.10 (m, 1H), 7.19 (d, |
| | | | | J = 8.4 Hz, 1H), 7.68 (s, 1H), |
| | | | | 8.37 (s, 1H), 8.41 (s, 1H), |
| | | | | 8.46 (s, 2H), 8.72 (s, 1H), |
| | | | | 9.05-9.07 (m, 2H), 10.48 (s, |
| | | | | 1H). |
| P-439 | A-57 | C-166 | 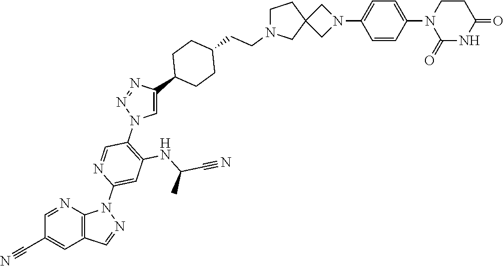 [SMILES: C[C@H](C#N)Nc1cc(-n2ncc3cc(C#N)cnc32)ncc1-n1cc([C@H]2CC[C@H](CCN3CCC4(C3)CN(c3ccc(N5CCC(=O)NC5=O)cc3)C4)CC2)nn1] | 18.2% yield [M + H]⁺ = 765.2 ¹H-NMR (400 MHz, DMSO d6): δ ppm 1.11-1.21 (m, 2H), 1.37-1.41 (m, 1H), 1.48-1.51 (m, 2H), 1.52-1.58 (m, 2H), 1.61 (d, J = 6.8 Hz, 3H), 1.86- 1.88 (m, 2H), 2.14-2.17 (m, 2H), 2.21-2.28 (m, 1H), 2.39- 2.43 (m, 2H), 2.67-2.69 (m, 2H), 2.71-2.77 (m, 1H), 3.12- 3.22 (m, 3H), 3.40-3.44 (m, 1H), 3.65-3.69 (m, 3H), 3.76- 3.79 (m, 1H), 3.85-3.90 (m, 3H), 4.99-5.03 (m, 1H), 6.47 (d, J = 8.8 Hz, 2H), 7.13 (d, |
| | | | | J = 8.4 Hz, 2H), 7.19 (d, J = |
| | | | | 9.2 Hz, 1H), 7.67 (s, 1H), |
| | | | | 8.36 (s, 1H), 8.41 (s, 1H), |
| | | | | 8.72 (s, 1H), 9.05-9.07 (m, |
| | | | | 2H), 10.24 (s, 1H). |
| P-440 | A-57 | C-167 | 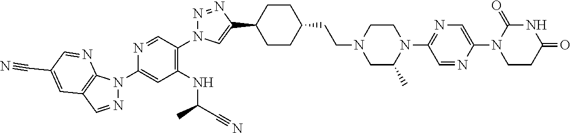 [SMILES: C[C@H](C#N)Nc1cc(-n2ncc3cc(C#N)cnc32)ncc1-n1cc([C@H]2CC[C@H](CCN3CCN(c4cnc(N5CCC(=O)NC5=O)cn4)[C@H](C)C3)CC2)nn1] | 10.2% yield [M + H]⁺ = 755.2 ¹H-NMR (400 MHz, DMSO- d₆): δ ppm 1.14-1.19 (m, 5H), 1.45-1.50 (m, 5H), 1.60 (d, J = 6.8 Hz, 3H), 1.88-1.91 (m, 2H), 2.13-2.16 (m, 3H), 2.67- 2.74 (m, 4H), 2.86-2.92 (m, 1H), 3.02-3.15 (m, 2H), 3.86 |
| | | | | (t, J = 6.8 Hz, 2H), 4.01-4.10 |
| | | | | (m, 1H), 4.55 (m, 1H), 4.99- |
| | | | | 5.10 (m, 1H), 7.19 (d, J = 8.0 |
| | | | | Hz, 1H), 7.67 (s, 1H), 8.07 |
| | | | | (s, 1H), 8.35-8.36 (m, 2H), |
| | | | | 8.41 (s, 1H), 8.71 (s, 1H), |
| | | | | 9.05-9.07 (m, 2H), 10.47 (s, |
| | | | | 1H). |
| | | | | Two protons are not apparent |
| P-441 | A-57 | C-149 | 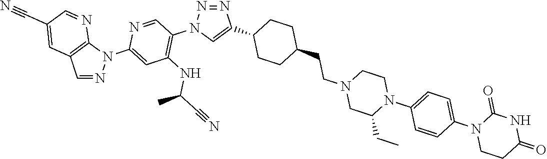 [SMILES: CC[C@@H]1CN(CC[C@H]2CC[C@H](c3cn(-c4cnc(-n5ncc6cc(C#N)cnc65)cc4N[C@H](C)C#N)nn3)CC2)CCN1c1ccc(N2CCC(=O)NC2=O)cc1] | 22.1% yield [M + H]⁺ = 767.4 ¹H-NMR (400 MHz, DMSO- d₆, VT): δ ppm 0.87-0.91 (m, 3H), 1.22-1.24 (m, 2H), 1.47- 1.56 (m, 3H), 1.63 (d, J = 7.20 Hz, 3H), 1.64-1.72 (m, 2H), 1.88-1.92 (m, 2H), 2.16- 2.19 (m, 2H), 2.69 (t, J = 6.8 Hz, 2H), 2.72-2.84 (m, 1H), |
| | | | | 3.17-3.36 (m, 5H), 4.90-4.92 |
| | | | | (m, 1H), 6.98-7.04 (m, 3H), |
| | | | | 7.23 (brs, 2H), 7.71 (s, 1H), |
| | | | | 8.28 (s, 1H), 8.43 (s, 1H), |
| | | | | 8.63 (s, 1H), 8.94 (d, J = 2.0 |
| | | | | Hz, 1H), 8.98 (d, J = 2.0 Hz, |
| | | | | 1H), 9.84 (s, 1H) |
| | | | | Eight protons are not |
| | | | | apparent |
| P-443 | A-57 | C-168 | 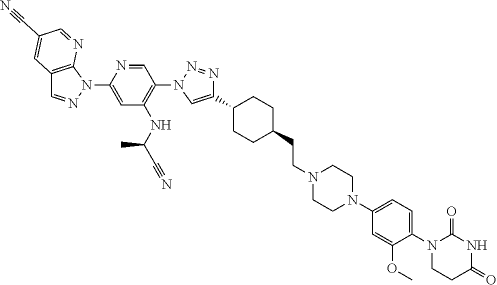 [SMILES: COc1cc(N2CCN(CC[C@H]3CC[C@H](c4cn(-c5cnc(-n6ncc7cc(C#N)cnc76)cc5N[C@H](C)C#N)nn4)CC3)CC2)ccc1N1CCC(=O)NC1=O] | 16.5% yield [M + H]⁺ = 769.2 ¹H-NMR (400 MHz, DMSO- d₆): δ ppm 1.18-1.21 (m, 2H), 1.33-1.53 (m, 3H), 1.61 (d, J = 6.8 Hz, 3H), 1.65-1.68 (m, 2H), 1.89-1.91 (m, 2H), 2.15- 2.18 (m, 2H), 2.64-2.67 (m, 2H), 2.71-2.82 (m, 1H), 3.05 (m, 2H), 3.14-3.19 (m, 2H), 3.26-3.32 (m, 2H), 3.51-3.53 (m, 2H), 3.61-3.64 (m, 2H), 3.80 (s, 3H), 3.91-3.94 (m, 2H), 4.96-5.05 (m, 1H), 6.56- 6.59 (m, 1H), 6.70 (d, J = 2.4 Hz, 1H), 7.10-7.12 (m, 1H), 7.19 (d, J = 8.0 Hz, 1H), 7.68 (s, 1H), 8.37 (s, 1H), 8.41 (s, |
| | | | | 1H), 8.72 (s, 1H), 9.05-9.07 |
| | | | | (m, 2H), 10.22 (s, 1H). |
| P-444 | A-57 | C-169 | 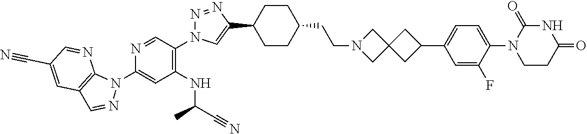 [SMILES: C[C@H](C#N)Nc1cc(-n2ncc3cc(C#N)cnc32)ncc1-n1cc([C@H]2CC[C@H](CCN3CC4(CC(c5ccc(N6CCC(=O)NC6=O)c(F)c5)C4)C3)CC2)nn1] | 17.6% yield [M + H]⁺ = 768.4 ¹H-NMR (400 MHz, DMSO- d₆): δ ppm 1.13-1.22 (m, 2H), 1.36-1.55 (m, 5H), 1.61 (d, J = 6.8 Hz, 3H), 1.83-1.86 (m, 2H), 2.13-2.16 (m, 2H), 2.32-2.45 (m, 2H), 2.59-2.63 (m, 1H), 2.72-2.79 (m, 3H), |
| | | | | 3.18-3.21 (m, 2H), 3.43-3.46 |
| | | | | (m, 2H), 3.97-3.99 (m, 1H), |
| | | | | 4.06-4.16 (m, 2H), 4.31-4.36 |
| | | | | (m, 1H), 4.99-5.03 (m, 1H), |
| | | | | 7.09-7.11 (m, 1H), 7.18-7.24 |
| | | | | (m, 2H), 7.33-7.37 (m, 1H), |
| | | | | 7.68 (brs, 1H), 8.36 (s, 1H), |
| | | | | 8.40 (s, 1H), 8.71 (s, 1H), |
| | | | | 9.06 (s, 2H), 10.45 (s, 1H). |
| | | | | Two protons are not apparent |
| | | | | ¹⁹F-NMR (376 MHz, DMSO- |
| | | | | d₆): δ ppm −120.830 |
| P-445 | A-57 | C-170 | 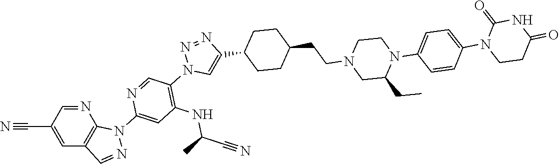 [SMILES: CC[C@H]1CN(CC[C@H]2CC[C@H](c3cn(-c4cnc(-n5ncc6cc(C#N)cnc65)cc4N[C@H](C)C#N)nn3)CC2)CCN1c1ccc(N2CCC(=O)NC2=O)cc1] | 17.8% yield [M + H]⁺ = 67.3 ¹H-NMR (400 MHz, DMSO- d₆-D2O exchange): δ 0.79- 0.90 (m, 3H), 1.12-1.22 (m, 2H), 1.37-1.55 (m, 4H), 1.63 (d, J = 6.8 Hz, 3H), 1.67-1.77 (m, 2H), 1.88-1.90 (m, 2H), 2.14-2.17 (m, 2H), 2.72-2.80 (m, 3H), 3.03-3.10 (m, 1H), |
| | | | | 3.20-3.27 (m, 3H), 3.51-3.60 |
| | | | | (m, 2H), 4.03-4.06 (m, 1H), |
| | | | | 4.99-5.04 (m, 1H), 6.95-6.98 |
| | | | | (m, 2H), 7.20-7.22 (m, 2H), |
| | | | | 7.71 (s, 1H), 8.34 (s, 1H), |
| | | | | 8.40 (s, 1H), 8.71 (s, 1H), |
| | | | | 9.01-9.05 (m, 2H). |
| | | | | Five protons are not apparent |
| P-446 | A-66 | C-154 | 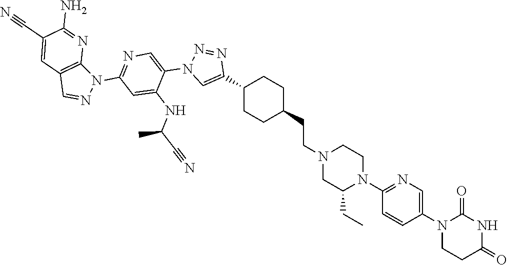 [SMILES: CC[C@@H]1CN(CC[C@H]2CC[C@H](c3cn(-c4cnc(-n5ncc6cc(C#N)c(N)nc65)cc4N[C@H](C)C#N)nn3)CC2)CCN1c1ccc(N2CCC(=O)NC2=O)cn1] | 16.4% yield [M + H]⁺ = 783.3 ¹H-NMR (400 MHz, DMSO- d₆): δ ppm 0.91 (t, J = 7.2 Hz, 3H), 1.12-1.22 (m, 2H), 1.33- 1.38 (m, 1H), 1.45-1.54 (m, 2H), 1.63 (d, J = 6.8 Hz, 3H), 1.67-1.71 (m, 2H), 1.77-1.81 (m, 1H), 1.87-1.89 (m, 2H), 2.15-2.17 (m, 2H), 2.68-2.79 (m, 3H), 2.93-3.02 (m, 1H), 3.09-3.13 (m, 4H), 3.54-3.59 (m, 2H), 3.72 (t, J = 6.8 Hz, 2H), 4.44-4.47 (m, 1H), 4.57- 4.61 (m, 1H), 4.97-5.02 (m, 1H), 6.95 (d, J = 7.2 Hz, 1H), 7.08-7.10 (m, 1H), 7.36 (brs, |
| | | | | 2H), 7.59 (dd, J = 2.8 Hz and |
| | | | | 9.2 Hz, 1H), 8.00 (s, 1H), |
| | | | | 8.12 (d, J = 2.4 Hz, 1H), 8.25 |
| | | | | (s, 1H), 8.32-8.34 (m, 2H), |
| | | | | 8.59 (s, 1H), 10.37 (s, 1H). |
| | | | | One proton is not apparent |
| P-447 | A-66 | C-149 | 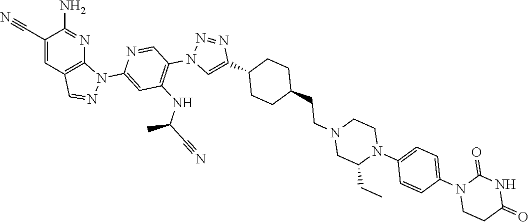 [SMILES: CC[C@@H]1CN(CC[C@H]2CC[C@H](c3cn(-c4cnc(-n5ncc6cc(C#N)c(N)nc65)cc4N[C@H](C)C#N)nn3)CC2)CCN1c1ccc(N2CCC(=O)NC2=O)cc1] | 20.9% yield [M + H]⁺ = 782.4 ¹H-NMR (400 MHz, DMSO- d₆, VT): δ ppm 0.82-0.93 (m, 3H), 1.18-1.26 (m, 2H), 1.42- 1.58 (m, 3H), 1.64-1.72 (m, 5H), 1.88-1.92 (m, 2H), 2.16- 2.19 (m, 2H), 2.69 (t, J = 6.4 Hz, 2H), 2.75-2.82 (m, 1H), 3.20-3.29 (m, 4H), 3.70-3.74 (m, 1H), 4.90-4.98 (m, 1H), 6.88-7.01 (m, 5H), 7.22 (brs, 2H), 7.92 (s, 1H), 8.19 (s, 1H), 8.25 (s, 1H), 8.35 (s, |
| | | | | 1H), 8.49 (s, 1H), 9.85 (s, |
| | | | | 1H) |
| | | | | Eight protons are not |
| | | | | apparent |
| P-449 | A-57 | C-171 | 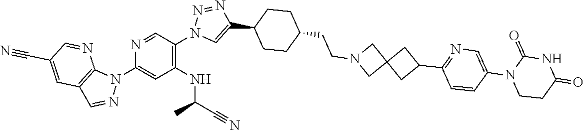 [SMILES: C[C@H](C#N)Nc1cc(-n2ncc3cc(C#N)cnc32)ncc1-n1cc([C@H]2CC[C@H](CCN3CC4(CC(c5ccc(N6CCC(=O)NC6=O)cn5)C4)C3)CC2)nn1] | 3% yield [M + H]⁺ = 751.2 ¹H-NMR (400 MHz, DMSO- d₆): δ ppm 1.11-1.14 (m, 2H), 1.41-1.48 (m, 5H), 1.61 (d, J = 6.80 Hz, 3H), 1.81-1.88 (m, 4H), 2.12-2.15 (m, 2H), 2.38-2.41 (m, 2H), 2.52-2.58 (m, 2H), 2.65-2.71 (m, 3H), |
| | | | | 2.80 (t, J = 6.8 Hz, 2H), 3.63 |
| | | | | (t, J = 5.6 Hz, 1H), 3.95 (t, |
| | | | | J = 6.4 Hz, 2H), 4.80 (s, 2H), |
| | | | | 4.99-5.00 (m, 1H), 7.20 (d, |
| | | | | J = 8.4 Hz, 1H), 7.68 (s, 1H), |
| | | | | 7.96 (d, J = 8.4 Hz, 1H), 8.30 |
| | | | | (brs, 1H), 8.41 (s, 1H), 8.46- |
| | | | | 8.49 (m, 1H), 8.72,(s, 1H), |
| | | | | 9.06-9.08 (m, 3H), 10.86 (s, |
| | | | | 1H). |
| P-455 | A-104 | C-150 | 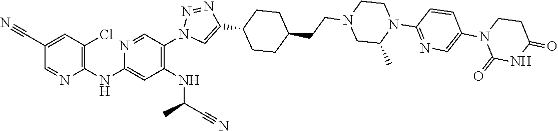 [SMILES: C[C@H](C#N)Nc1cc(Nc2ncc(C#N)cc2Cl)ncc1-n1cc([C@H]2CC[C@H](CCN3CCN(c4ccc(N5CCC(=O)NC5=O)cn4)[C@H](C)C3)CC2)nn1] | 25.8% yield [M + H]⁺ = 763.5 ¹H-NMR (400 MHz, DMSO- d₆): δ ppm 1.12-1.18 (m, 2H), 1.23 (d, J = 6,8 Hz, 3H), 1.33-1.39 (m, 1H), 1.44-1.53 (m, 2H), 1.60 (d, J = 7.2 Hz, 3H), 1.63-1.71 (m, 2H), 1.86- |
| | | | | 1.89 (m, 2H), 2.12-2.15 (m, |
| | | | | 2H), 2.69-2.73 (m, 3H), 2.98- |
| | | | | 3.07 (m, 1H), 3.17-3.23 (m, |
| | | | | 4H), 3.72 (t, J = 7.2 Hz, 2H), |
| | | | | 4.33-4.37 (m, 1H), 4.78-4.85 |
| | | | | (m, 2H), 6.93 (d, J = 9.2 Hz, |
| | | | | 1H), 7.05 (d, J = 7.6 Hz, 1H), |
| | | | | 7.61 (dd, J = 2.4, 8.8 Hz, 1H), |
| | | | | 7.73 (brs, 1H), 8.14 (d, J = |
| | | | | 2.8 Hz, 1H), 8.17 (m, 1H), |
| | | | | 8.27 (s, 1H), 8.48-8.50 (m, |
| | | | | 1H), 8.65 (brs, 1H), 9.40 (brs, |
| | | | | 1H), 10.37 (s, 1H). |
| | | | | Two protons are not apparent |
| P-458 | A-57 | C-172 | 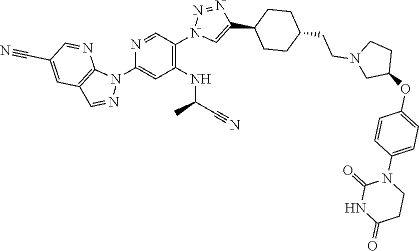 [SMILES: C[C@H](C#N)Nc1cc(-n2ncc3cc(C#N)cnc32)ncc1-n1cc([C@H]2CC[C@H](CCN3CC[C@@H](Oc4ccc(N5CCC(=O)NC5=O)cc4)C3)CC2)nn1] | 27.3% yield [M + H]⁺ = 740 ¹H-NMR (400 MHz, DMSO- d₆, VT): δ ppm 1.17-1.21 (m, 2H), 1.49-1.55 (m, 3H), 1.63- 1.70 (m, 5H), 1.86-1.90 (m, 2H), 2.15-2.18 (m, 3H), 2.70 (t, J = 6.8 Hz, 2H), 2.75-2.82 (m, 1H), 3.22-3.34 (m, 4H), 3.73 (t, J = 6.8 Hz, 2H), 4.90- 4.94 (m, 1H), 5.15 (m, 1H), 6.98-7.04 (m, 3H), 7.27-7.30 (m, 2H), 7.71 (s, 1H), 8.27 (s, 1H), 8.43 (s, 1H), 8.63 (s, 1H), 8.94 (d, J = 2.0 Hz, 1H), 8.98 (d, J = 2.0 Hz, 1H), 9.88 |
| | | | | (s, 1H) |
| | | | | Three protons are not |
| | | | | apparent |
| P-459 | A-57 | C-173 | 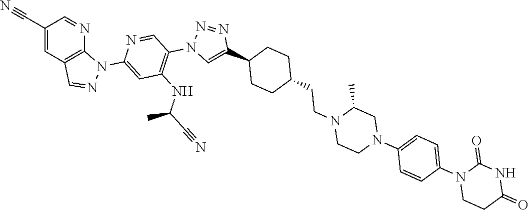 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)cc4)C[C@H]3C)CC2)nn1] | 25.7% yield [M + H]⁺ = 753.4 ¹H-NMR (400 MHz, DMSO- d₆): δ ppm 1.12-1.25 (m, 2H), 1.39 (d, J = 6.4 Hz, 3H), 1.40- 1.71 (m, 9H), 1.89-1.92 (m, 2H), 2.14-2.17 (m, 2H), 2.68- 2.71 (m, 2H), 2.78-2.85 (m, 1H), 2.97-3.03 (m, 1H), 3.11- 3.25 (m, 2H), 3.36-3.47 (m, 2H), 3.67-3.73 (m, 2H), 3.83- 3.91 (m, 2H), 4.97-5.01 (m, 1H), 6.95-7.08 (m, 2H), 7.18- |
| | | | | 7.24 (m, 3H), 7.68 (s, 1H), |
| | | | | 8.37 (s, 1H), 8.41 (s, 1H), |
| | | | | 8.72 (s, 1H), 9.05-9.07 (m, |
| | | | | 2H), 10.29 (s, 1H). |
| | | | | One proton is not apparent |
| P-461 | A-57 | C-181b | 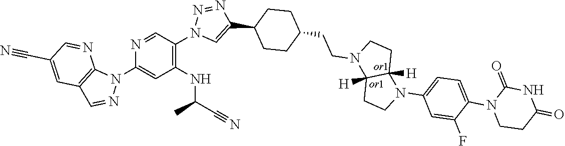 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc(N4CCC(=O)NC4=O)c(F)c3)CC2)nn1] | 25.8% yield [M + H]⁺ = 783.5 ¹H-NMR (400 MHz, DMSO- d₆): δ ppm 1.16-1.22 (m, 2H), 1.42-1.55 (m, 3H), 1.61 (d, J = 6.8 Hz, 3H), 1.66-1.68 (m, 3H), 1.89-1.91 (m, 2H), 2.15- 2.18 (m, 2H), 2.32-2.35 (m, 3H), 2.62-2.78 (m, 4H), 3.20- |
| | | | | 3.30 (m, 3H), 3.52-3.60 (m, |
| | | | | 1H), 3.62 (t, J = 6.8 Hz, 2H), |
| | | | | 3.65-3.70 (m, 1H), 4.15-4.30 |
| | | | | (m, 1H), 4.50-4.58 (m, 1H), |
| | | | | 4.98-5.07 (m, 1H), 6.48-6.58 |
| | | | | (m, 2H), 7.18-7.26 (m, 2H), |
| | | | | 7.68 (s, 1H), 8.37-8.41 (m, |
| | | | | 2H), 8.72 (s, 1H), 9.05 (s, |
| | | | | 2H), 10.36 (s, 1H). |
| | | | | ¹⁹F-NMR (376 MHz, DMSO- |
| | | | | d₆): δ ppm −120.405 |
| P-463 | A-66 | C-150 | 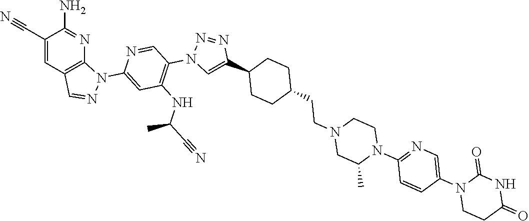 [SMILES: C[C@H](C#N)Nc1cc(-n2ncc3cc(C#N)c(N)nc32)ncc1-n1cc([C@H]2CC[C@H](CCN3CCN(c4ccc(N5CCC(=O)NC5=O)cn4)[C@H](C)C3)CC2)nn1] | 34.9% yield [M + H]⁺ = 769.4 ¹H-NMR (400 MHz, DMSO- d₆): δ ppm 1.19-1.25 (m, 5H), 1.31-1.42 (m, 1H), 1.49-1.53 (m, 2H), 1.63-1.69 (m, 5H), 1.87-1.91 (m, 2H), 2.15-2.18 (m, 2H), 2.67-2.78 (m, 3H), 2.95-3.08 (m, 1H), 3.19-3.24 (m, 4H), 3.54-3.63 (m, 2H), 3.72 (t, J = 6.8 Hz, 2H), 4.35- 4.38 (m, 1H), 4.73-4.85 (m, 1H), 4.99-5.01 (m, 1H), 6.94 (d, J = 9.2 Hz, 1H), 7.09 (d, |
| | | | | J = 8.4 Hz, 1H), 7.36 (brs, |
| | | | | 2H), 7.60-7.63 (m, 1H), 8.01 |
| | | | | (s, 1H), 8.14 (s, 1H), 8.25 (s, |
| | | | | 1H), 8.33 (s, 1H), 8.35 (s, |
| | | | | 1H), 8.59 (s, 1H), 10.38 (s, |
| | | | | 1H) |
| P-464 | A-57 | C-185b | 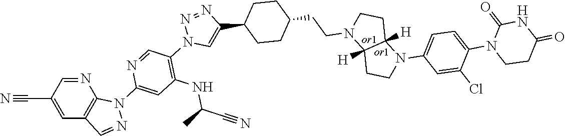 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc(N4CCC(=O)NC4=O)c(Cl)c3)CC2)nn1] | 11.1% yield [M + H]⁺ = 799.4 ¹H-NMR (400 MHz, DMSO- d₆): δ ppm 1.21-1.24 (m, 2H), 1.50-1.53 (m, 3H), 1.61-1.68 (m, 5H), 1.89-1.92 (m, 2H), 2.16-2.19 (m, 2H), 2.71-2.78 (m, 3H), 3.24-3.32 (m, 3H), 3.45-3.62 (m, 3H), 4.27-4.32 |
| | | | | (m, 1H), 4.51-4.62 (m, 1H), |
| | | | | 5.01-5.05 (m, 1H), 6.69 (d, |
| | | | | J = 2.4 Hz, 1H), 6.78 (s, 1H), |
| | | | | 7.19 (d, J = 8.4 Hz, 1H), 7.29 |
| | | | | (d, J = 5.6 Hz, 1H), 7.68 (s, |
| | | | | 1H), 8.38 (s, 1H), 8.42 (s, |
| | | | | 1H), 8.73 (s, 1H), 9.06-9.08 |
| | | | | (m, 2H), 10.37 (s, 1H) |
| | | | | Six protons are not apparent |
| P-465 | A-57 | C-174 | 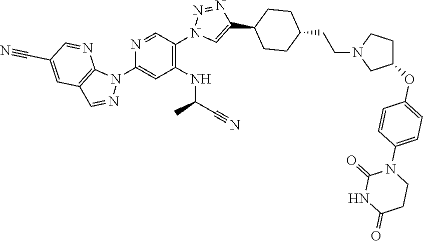 [SMILES: C[C@H](C#N)Nc1cc(-n2ncc3cc(C#N)cnc32)ncc1-n1cc([C@H]2CC[C@H](CCN3CC[C@H](Oc4ccc(N5CCC(=O)NC5=O)cc4)C3)CC2)nn1] | 16.8% yield [M + H]⁺ = 740.3 ¹H-NMR (400 MHz, DMSO- d₆): δ 1.15-1.21 (m, 2H), 1.40-1.51 (m, 3H), 1.60-1.65 (m, 5H), 1.85-1.88 (m, 2H), 2.05-2.25 (m, 3H), 2.67-2.75 (m, 3H), 3.21-3.30 (m, 4H), 3.72-3.75 (m, 4H), 5.01-5.05 (m, 1H), 5.10-5.21 (m, 1H), 7.00-7.02 (m, 2H), 7.19-7.23 (m, 1H), 7.29 (d, J = 8.80 Hz, 2H), 7.67 (s, 1H), 8.37 (s, 1H), 8.41 (s, 1H), 8.73 (s, 1H), 9.07-9.08 (m, 2H), 10.35 |
| | | | | (s, 1H). |
| | | | | One proton is not apparent |
| P-468 | A-57 | C-175 | 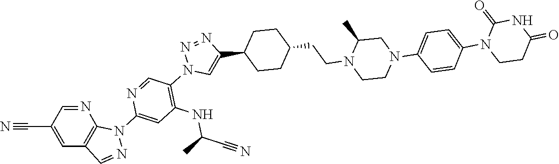 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)cc4)C[C@@H]3C)CC2)nn1] | 4.6% yield [M + H]+ = 753.2 1H-NMR (400 MHz, DMSO- d₆): δ ppm 1.20-1.24 (m, 3H), 1.39 (d, J = 6.4 Hz, 3H), 1.47- 1.53 (m, 2H), 1.62 (d, J = 6.8 Hz, 3H), 1.65-1.75 (m, 1H), 1.90-1.93 (m, 2H), 2.16-2.19 (m, 2H), 2.70-2.72 (m, 2H), 2.77-2.80 (m, 2H), 2.98-3.05 (m, 1H), 3.17-3.25 (m, 2H), |
| | | | | 3.65-3.74 (m, 3H), 3.81-3.95 |
| | | | | (m, 2H), 5.01-5.05 (m, 1H), |
| | | | | 7.01-7.08 (m, 2H), 7.19-7.24 |
| | | | | (m, 3H), 7.68 (s, 1H), 8.38 (s, |
| | | | | 1H), 8.42 (s, 1H), 8.73 (s, |
| | | | | 1H), 9.06-9.07 (s, 2H), 10.29 |
| | | | | (s, 1H) |
| | | | | Three protons are not |
| | | | | apparent |
| P-470 | A-57 | C-186a | 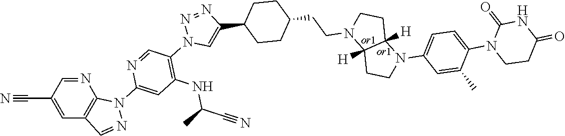 | 8.5% yield [M + H]⁺ = 779.4 ¹H-NMR (400 MHz, DMSO- d₆): δ ppm 1.15-1.23 (m, 2H), 1.40-1.55 (m, 4H), 1.62 (d, J = 6.8 Hz, 3H), 1.65-1.69 (m, 2H), 1.89-1.92 (m, 2H), 2.16- 2.19 (m, 4H), 2.28-2.29 (s, 3H), 2.69-2.71 (m, 2H), 2.74- |
| | | | | 2.80 (m, 1H), 2.85-2.91 (m, |
| | | | | 1H), 3.18-3.24 (m, 2H), 3.31- |
| | | | | 3.37 (m, 1H), 3.51-3.58 (m, |
| | | | | 1H), 3.73 (t, J = 6.8 Hz, 2H), |
| | | | | 4.15-4.20 (m, 1H), 4.41-4.46 |
| | | | | (m, 1H), 4.99-5.05 (m, 1H), |
| | | | | 7.01-7.21 (m, 4H), 7.68 (s, |
| | | | | 1H), 8.38 (s, 1H), 8.42 (s, |
| | | | | 1H), 8.73 (s, 1H), 9.07-9.08 |
| | | | | (m, 2H), 10.31 (s, 1H). |
| | | | | Two protons are not apparent |
| P-471 | A-57 | C-176 | 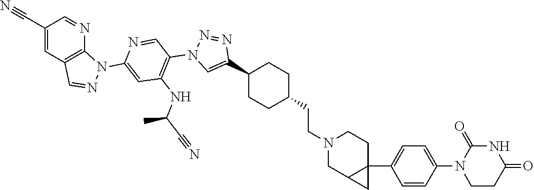 [SMILES: C[C@H](C#N)Nc1cc(-n2ncc3cc(C#N)cnc32)ncc1-n1cc([C@H]2CC[C@H](CCN3CCC4(c5ccc(N6CCC(=O)NC6=O)cc5)CC4C3)CC2)nn1] | 15.1% yield [M + H]⁺ = 750.4 ¹H-NMR (500 MHz, DMSO- d₆) δ 10.40 (s, 1H), 9.13-9.09 (m, 2H), 8.76 (s, 1H), 8.45 (s, 1H), 8.41 (s, 1H), 7.72 (s, 1H), 7.43 (d, J = 8.5 Hz, 2H), 7.33 (d, J = 8.3 Hz, 1H), 7.24 (d, J = 8.3 Hz, 1H), 5.06 (br quin, J = 7.3 Hz, 1H), 3.95 (s, 2H), 3.81 (t, J = 6.7 Hz, 2H), 3.22 (d, J = 4.8 Hz, 5H), 2.84- |
| | | | | 2.72 (m, 3H), 2.35 (br s, 2H), |
| | | | | 2.20 (br d, J = 11.4 Hz, 2H), |
| | | | | 1.92 (br d, J = 11.0 Hz, 2H), |
| | | | | 1.66 (d, J = 6.8 Hz, 5H), 1.53 |
| | | | | (q, J = 12.7 Hz, 3H), 1.43 (br |
| | | | | s, 1H), 1.28-1.11 (m, 4H) |
| P-473 | A-104 | C-171 | 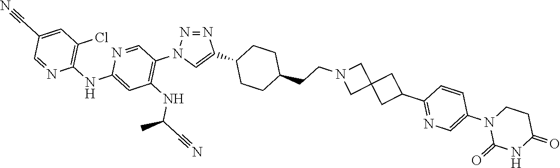 [SMILES: C[C@H](C#N)Nc1cc(Nc2ncc(C#N)cc2Cl)ncc1-n1cc([C@H]2CC[C@H](CCN3CC4(CC(c5ccc(N6CCC(=O)NC6=O)cn5)C4)C3)CC2)nn1] | 9.8% yield [M + H]⁺ = 760.2 ¹H-NMR (400 MHz, DMSO- d₆): δ ppm 1.02-1.13 (m, 2H), 1.23-1.29 (m, 3H), 1.40-1.46 (m, 3H), 1.58-1.60 (m, 3H), 1.63-1.70 (m, 1H), 1.81-1.86 (m, 4H), 2.07-2.12 (m, 2H), 2.36-2.40 (m, 1H), 2.58-2.81 (m, 4H), 3.53-3.63 (m, 1H), 3.79-3.82 (m, 2H), 4.76-4.82 |
| | | | | (m, 2H), 6.86 (d, J = 8.0 Hz, |
| | | | | 1H), 7.26 (d, J = 8.4 Hz, 1H), |
| | | | | 7.65 (dd, J = 2.8 Hz and 8.4 |
| | | | | Hz, 1H), 7.75 (s, 1H), 8.13 |
| | | | | (s, 1H), 8.24 (s, 1H), 8.45- |
| | | | | 8.50 (m, 2H), 8.63 (s, 1H), |
| | | | | 9.07 (s, 1H), 10.46 (s, 1H). |
| | | | | Three protons are not |
| | | | | apparent |
| P-474 | A-57 | C-186b | 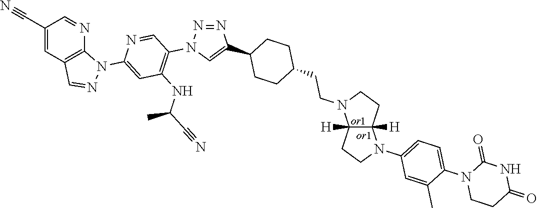 [SMILES: Cc1cc(N2CC[C@H]3[C@@H]2CCN3CC[C@H]2CC[C@H](c3cn(-c4cnc(-n5ncc6cc(C#N)cnc65)cc4N[C@H](C)C#N)nn3)CC2)ccc1N1CCC(=O)NC1=O] | 11.4% yield [M + H]⁺ = 779.2 ¹H-NMR (400 MHz, DMSO- d₆): δ ppm 1.14-1.24 (m, 2H), 1.40-1.56 (m, 4H), 1.62 (d, J = 7.2 Hz, 3H), 1.64-1.69 (m, 2H), 1.89-1.92 (m, 2H), 2.16-2.19 (m, 4H), 2.27-2.29 (m, 4H), 2.68-2.71 (m, 2H), 2.73-2.90 (m, 2H), 3.18-3.24 (m, 2H), 3.56-3.63 (m, 2H), 3.74 (t, J = 7.2 Hz, 2H), 4.15- 4.44 (m, 2H), 4.99-5.06 (m, |
| | | | | 1H), 7.02 (d, J = 8.0 Hz, 1H), |
| | | | | 7.09-7.16 (m, 2H), 7.18-7.21 |
| | | | | (m, 1H), 7.68 (s, 1H), 8.38 (s, |
| | | | | 1H), 8.42 (s, 1H), 8.73 (s, |
| | | | | 1H), 9.06-9.08 (m, 2H), |
| | | | | 10.32 (s, 1H). |
| | | | | One proton is not apparent |
| P-483 | A-57 | C-177 | 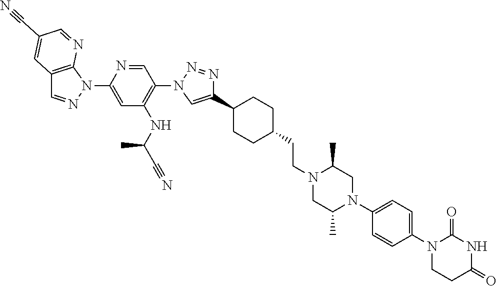 [SMILES: C[C@H](C#N)Nc1cc(-n2ncc3cc(C#N)cnc32)ncc1-n1cc([C@H]2CC[C@H](CCN3C[C@@H](C)N(c4ccc(N5CCC(=O)NC5=O)cc4)C[C@@H]3C)CC2)nn1] | 17.6% yield [M + H]⁺ = 767.2 ¹H-NMR (400 MHz, DMSO- d₆): δ ppm 1.00 (brs, 3H), 1.21-1.27 (m, 3H), 1.33 (d, J = 6.4 Hz, 3H), 1.46-1.59 (m, 3H), 1.63-1.69 (m, 5H), 1.91- 1.94 (m, 2H), 2.16-2.19 (m, 2H), 2.70 (t, J = 6.8 Hz, 2H), 2.76-2.82 (m, 1H), 3.31-3.35 (m, 2H), 3.49-3.61 (m, 2H), 3.74 (t, J = 6.4 Hz, 2H), 4.92- 4.96 (m, 1H), 7.07-7.09 (m, 3H), 7.27 (brs, 2H), 7.71 (s, 1H), 8.30 (s, 1H), 8.43 (s, 1H), 8.65 (s, 1H), 8.97-9.00 (m, 2H), 10.00 (s, 1H). Three protons are not apparent |
| P-476 | A-57 | C-178 | 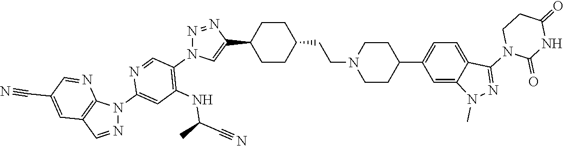 [SMILES: C[C@H](C#N)Nc1cc(-n2ncc3cc(C#N)cnc32)ncc1-n1cc([C@H]2CC[C@H](CCN3CCC(c4ccc5c(N6CCC(=O)NC6=O)nn(C)c5c4)CC3)CC2)nn1] | 17.7% yield [M + H]⁺ = 792.2 ¹H-NMR (400 MHz, DMSO- d₆): δ ppm 1.14-1.23 (m, 2H), 1.40-1.57 (m, 3H), 1.62 (d, J = 7.2 Hz, 3H), 1.65-1.69 (m, 2H), 1.89-2.01 (m, 4H), 2.11-2.20 (m, 4H), 2.75-2.78 (m, 3H), 2.98-3.02 (m, 1H), |
| | | | | 3.07-3.14 (m, 2H), 3.19-3.23 |
| | | | | (m, 2H), 3.65-3.68 (m, 2H), |
| | | | | 3.93 (t, J = 6.8 Hz, 2H), 4.00- |
| | | | | 4.01 (s, 3H), 4.99-5.06 (m, |
| | | | | 1H), 7.05 (d, J = 8.8 Hz, 1H), |
| | | | | 7.20 (d, J = 8.4 Hz, 1H), 7.43 |
| | | | | (s, 1H), 7.62-7.65 (m, 1H), |
| | | | | 7.68 (s, 1H), 8.38 (s, 1H), |
| | | | | 8.42 (s, 1H), 8.73 (s, 1H), |
| | | | | 9.06-9.08 (m, 2H), 10.56 (s, |
| | | | | 1H). |
| P-477 | A-57 | C-179 | 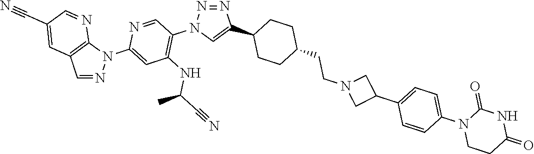 [SMILES: C[C@H](C#N)Nc1cc(-n2ncc3cc(C#N)cnc32)ncc1-n1cc([C@H]2CC[C@H](CCN3CC(c4ccc(N5CCC(=O)NC5=O)cc4)C3)CC2)nn1] | 16.7% yield [M + H]⁺ = 710.1 ¹H-NMR (400 MHz, DMSO- d₆-VT): δ ppm 1.12-1.24 (m, 2H), 1.40-1.57 (m, 5H), 1.64 (d, J = 6.8 Hz, 3H), 1.88 (m, 2H), 2.16-2.18 (m, 2H), 2.70 (d, J = 6.8 Hz, 2H), 2.77-2.81 (m, 1H), 3.78 (t, J = 6.8 Hz, 2H), 4.09-4.13 (m, 2H), 4.31- |
| | | | | 4.38 (m, 1H), 4.46-4.48 (m, |
| | | | | 1H), 4.88-4.96 (m, 1H), 7.05 |
| | | | | (d, J = 8.0 Hz, 1H), 7.35-7.43 |
| | | | | (m, 4H), 7.70 (s, 1H), 8.29 (s, |
| | | | | 1H), 8.43 (s, 1H), 8.64 (s, |
| | | | | 1H), 8.96 (d, J = 2.0 Hz, 1H), |
| | | | | 8.99 (d, J = 2.0 Hz, 1H), |
| | | | | 10.00 (s, 1H). |
| | | | | Three protons are not |
| | | | | apparent |
| P-478 | A-57 | C-185a | 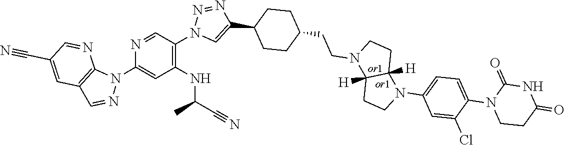 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc(N4CCC(=O)NC4=O)c(Cl)c3)CC2)nn1] | 3.9% yield [M + H]⁺ = 799.2 ¹H-NMR (400 MHz, DMSO- d₆): δ ppm 1.11-1.21 (m, 2H), 1.49-1.55 (m, 4H), 1.61-1.67 (m, 5H), 1.87-1.90 (m, 2H), 2.15-2.27 (m, 4H), 2.68-2.77 (m, 3H), 3.06-3.12 (m, 1H), 3.17-3.21 (m, 2H), 3.59-3.61 |
| | | | | (m, 2H), 3.72-3.77 (m, 3H), |
| | | | | 4.16-4.27 (m, 1H), 4.84-4.86 |
| | | | | (m, 1H), 4.95-5.02 (m, 1H), |
| | | | | 7.24-7.27 (m, 3H), 7. 43-7.44 |
| | | | | (m, 1H), 7.68 (s, 1H), 8.37 (s, |
| | | | | 1H), 8.41 (s, 1H), 8.72 (s, |
| | | | | 1H), 9.06-9.07 (s, 2H), 10.37 |
| | | | | (s, 1H) |
| | | | | One proton is not apparent |
| P-480 | A-57 | C-181a | 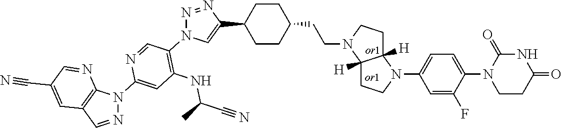 [SMILES: C[C@H](C#N)Nc1cc(-n2ncc3cc(C#N)cnc32)ncc1-n1cc([C@H]2CC[C@H](CCN3CC[C@H]4[C@@H]3CCN4c3ccc(N4CCC(=O)NC4=O)c(F)c3)CC2)nn1] | 14.8% yield [M + H]⁺ = 783.1 ¹H-NMR (400 MHz, DMSO- d₆): δ ppm 1.14-1.22 (m, 2H), 1.39-1.53 (m, 3H), 1.62 (d, J = 7.2 Hz, 3H), 1.64-1.69 (m, 2H), 1.88-1.91 (m, 2H), 2.15-2.17 (m, 2H), 2.27-2.38 |
| | | | | (m, 2H), 2.65-2.70 (m, 4H), |
| | | | | 3.21-3.27 (m, 2H), 3.48-3.53 |
| | | | | (m, 2H), 3.61 (t, J = 6.8 Hz, |
| | | | | 2H), 3.65-3.72 (m, 1H), 4.19- |
| | | | | 4.30 (m, 1H), 4.52-4.56 (m, |
| | | | | 1H), 4.96-5.00 (m, 1H), 6.49- |
| | | | | 6.58 (m, 2H), 7.18-7.26 (m, |
| | | | | 2H), 7.71 (brs, 1H), 8.37 (s, |
| | | | | 1H), 8.41 (brs, 1H), 8.72 (brs, |
| | | | | 1H), 9.06 (s, 2H), 10.36 (s, |
| | | | | 1H). |
| | | | | Two protons are not apparent |
| | | | | ¹⁹F-NMR (376 MHz, DMSO- |
| | | | | d₆): δ ppm −120.383 |
| P-486 | A-57 | C-118 | 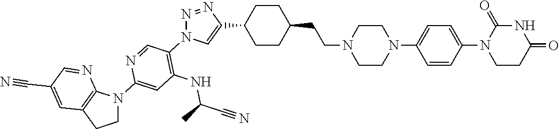 [SMILES: C[C@H](C#N)Nc1cc(N2CCc3cc(C#N)cnc32)ncc1-n1cc([C@H]2CC[C@H](CCN3CCN(c4ccc(N5CCC(=O)NC5=O)cc4)CC3)CC2)nn1] | 12.3% yield [M + H]⁺ = 740.2 ¹H-NMR (400 MHz, DMSO- d₆): δ 1.16-1.24 (m, 3H), 1.37-1.51 (m, 3H), 1.63-1.65 (m, 5H), 1.87-1.89 (m, 2H), 2.13-2.16 (m, 2H), 2.69-2.74 (m, 3H), 2.98-3.01 (m, 2H), |
| | | | | 3.15-3.27 (m, 6H), 3.72 (t, |
| | | | | J = 6.8 Hz, 2H), 3.85-3.88 |
| | | | | (m, 2H), 4.32-4.37 (m, 2H), |
| | | | | 4.71-4.74 (m, 1H), 6.79 (d, |
| | | | | J = 7.6 Hz, 1H), 7.05 (d, J = |
| | | | | 9.2 Hz, 2H), 7.21-7.24 (m, |
| | | | | 2H), 7.92 (s, 1H), 8.17 (s, |
| | | | | 1H), 8.24 (s, 1H), 8.45 (s, |
| | | | | 1H), 8.52 (s, 1H), 10.29 (s, |
| | | | | 1H) |
| | | | | One proton is not apparent |
| P-491 | A-57 | C-157b | 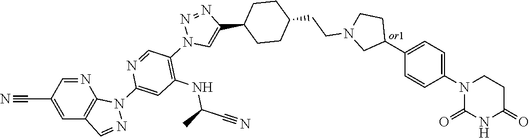 [SMILES: C[C@H](C#N)Nc1cc(-n2ncc3cc(C#N)cnc32)ncc1-n1cc([C@H]2CC[C@H](CCN3CCC(c4ccc(N5CCC(=O)NC5=O)cc4)C3)CC2)nn1] | 12.5% yield [M + H]⁺ = 724.2 ¹H-NMR (400 MHz, DMSO- d₆): δ ppm 1.12-1.26 (m, 2H), 1.43-1.53 (m, 3H), 1.61-1.63 (m, 5H), 1.87-1.91 (m, 2H), 2.15-2.18 (m, 2H), 2.39-2.43 (m, 1H), 2.67-2.74 (m, 3H), 3.10-3.20 (m, 1H), 3.61-3.80 |
| | | | | (m, 5H), 5.01-5.05 (m, 1H), |
| | | | | 7.18-7.22 (m, 1H), 7.33-7.42 |
| | | | | (m, 4H), 7.68 (s, 1H), 8.37 (s, |
| | | | | 1H), 8.42 (s, 1H), 8.73 (s, |
| | | | | 1H), 9.06-9.08 (m, 2H), |
| | | | | 10.37 (s, 1H) |
| | | | | Four protons are not apparent |
| P-494 | A-57 | C-188a | 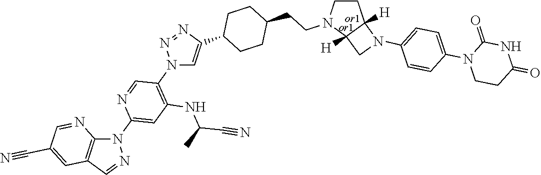 [SMILES: C[C@H](C#N)Nc1cc(-n2ncc3cc(C#N)cnc32)ncc1-n1cc([C@H]2CC[C@H](CCN3CC[C@H]4[C@@H]3CN4c3ccc(N4CCC(=O)NC4=O)cc3)CC2)nn1] | 21.9% yield [M + H]⁺ = 751.2 ¹H-NMR (400 MHz, DMSO- d₆): δ ppm 1.14-1.23 (m, 2H), 1.40-1.54 (m, 4H), 1.62 (d, J = 6.8 Hz, 3H), 1.64-1.68 (m, 1H), 1.90-1.92 (m, 3H), 2.16- 2.25 (m, 3H), 2.68-2.70 (m, 2H), 2.72-2.80 (m, 1H), 3.17- 3.21 (m, 1H), 3.68 (t, J = 6.8 Hz, 3H), 3.90-4.06 (m, 2H), |
| | | | | 4.23-4.25 (m, 1H), 4.66 (m, |
| | | | | 1H), 4.77-4.80 (m, 1H), 4.99- |
| | | | | 5.04 (m, 1H), 6.51-6.55 (m, |
| | | | | 2H), 7.15-7.20 (m, 3H), 7.68 |
| | | | | (s, 1H), 8.38 (s, 1H), 8.42 (s, |
| | | | | 1H), 8.73 (s, 1H), 9.06-9.07 |
| | | | | (m, 2H), 10.25 (s, 1H). |
| | | | | One proton is not apparent |
| P-495 | A-57 | C-188b | 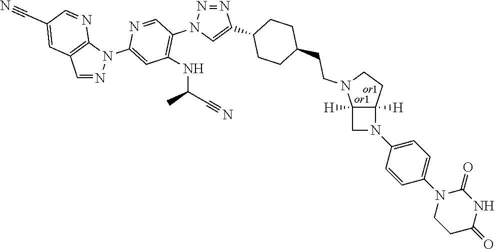 [SMILES: C[C@H](C#N)Nc1cc(-n2ncc3cc(C#N)cnc32)ncc1-n1cc([C@H]2CC[C@H](CCN3CC[C@@H]4[C@H]3CN4c3ccc(N4CCC(=O)NC4=O)cc3)CC2)nn1] | 15.7% yield [M + H]⁺ = 751.2 ¹H-NMR (400 MHz, DMSO- d₆-VT): δ ppm 1.15-1.24 (m, 2H), 1.52-1.61 (m, 6H), 1.64 (d, J = 7.2 Hz, 3H), 1.89-1.92 (m, 2H), 2.02-2.07 (m, 1H), 2.15-2.25 (m, 3H), 2.65-2.70 (m, 3H), 3.21-3.29 (m, 2H), 3.67 (t, J = 6.8 Hz, 2H), 3.71- 3.74 (m, 1H), 3.92-3.96 (m, 1H), 4.10-4.13 (m, 1H), 4.51- 4.55 (m, 1H), 4.77-4.79 (m, 1H), 4.88-4.93 (m, 1H), 6.49 (d, J = 8.8 Hz, 2H), 7.03 (d, |
| | | | | J = 8.0 Hz, 1H), 7.15 (d, J = |
| | | | | 8.8 Hz, 2H), 7.72 (s, 1H), |
| | | | | 8.28 (s, 1H), 8.43 (s, 1H), |
| | | | | 8.63 (s, 1H), 8.94 (s, 1H), |
| | | | | 8.98 (s, 1H), 9.78 (s, 1H). |
| P-496 | A-57 | C-182 | 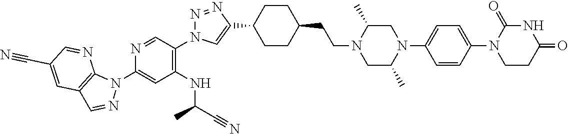 [SMILES: C[C@H](C#N)Nc1cc(-n2ncc3cc(C#N)cnc32)ncc1-n1cc([C@H]2CC[C@H](CCN3C[C@@H](C)N(c4ccc(N5CCC(=O)NC5=O)cc4)C[C@H]3C)CC2)nn1] | 7.3% yield [M + H]⁺ = 767.2 ¹H-NMR (400 MHz, DMSO- d₆, VT): δ ppm 1.12-1.28 (m, 5H), 1.49-1.67 (m, 11H), 1.90-1.93 (m, 2H), 2.16-2.19 (m, 2H), 2.69 (t, J = 6.8 Hz, 2H), 2.73-2.82 (m, 1H), 3.04-3.08 (m, 1H), 3.14-3.15 |
| | | | | (m, 2H), 3.72 (t, J = 6.8 Hz, |
| | | | | 2H), 4.90-4.94 (m, 1H), |
| | | | | 7.03-7.05 (m, 3H), 7.22- |
| | | | | 7.24 (m, 2H), 7.51-7.80 (m, |
| | | | | 1H), 8.28 (s, 1H), 8.39- |
| | | | | 8.65 (m, 2H), 8.94 (d, J = |
| | | | | 1.6 Hz, 1H), 8.98 (s, 1H), |
| | | | | 9.84 (s, 1H). |
| | | | | Five protons are not apparent |
| P-497 | A-57 | C-157a | 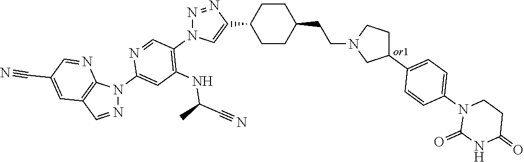 [SMILES: C[C@H](C#N)Nc1cc(-n2ncc3cc(C#N)cnc32)ncc1-n1cc([C@H]2CC[C@H](CCN3CCC(c4ccc(N5CCC(=O)NC5=O)cc4)C3)CC2)nn1] | 24.1% yield [M + H]⁺ = 724.6 ¹H-NMR (400 MHz, DMSO- d₆): δ ppm 1.14-1.22 (m, 2H), 1.41-1.58 (m, 3H), 1.62 (d, J = 6.80 Hz, 3H), 1.63-1.68 (m, 2H), 1.87-1.91 (m, 2H), 2.15-2.18 (m, 3H), 2.38-2.46 (m, 1H), 2.69 (t, J = 6.8 0 Hz, 2H), 2.76-2.82 (m, 1H), 3.11- |
| | | | | 3.23 (m, 1H), 3.28-3.36 (m, |
| | | | | 2H), 3.68-3.72 (m, 2H), 3.78 |
| | | | | (t, J = 6.80 Hz, 2H), 3.91-3.96 |
| | | | | (m, 1H), 4.99-5.06 (m, 1H), |
| | | | | 7.19 (d, J = 8.00 Hz, 1H), |
| | | | | 7.33-7.39 (m, 4H), 7.68 (s, |
| | | | | 1H), 8.37 (s, 1H), 8.42 (s, |
| | | | | 1H), 8.73 (s, 1H), 9.06-9.08 |
| | | | | (m, 2H), 10.38 (s, 1H). |
| | | | | One proton is not apparent |
| P-498 | A-57 | C-183 | 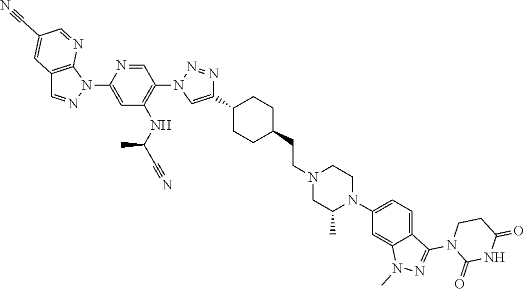 [SMILES: C[C@H](C#N)Nc1cc(-n2ncc3cc(C#N)cnc32)ncc1-n1cc([C@H]2CC[C@H](CCN3CCN(c4ccc5c(N6CCC(=O)NC6=O)nn(C)c5c4)[C@H](C)C3)CC2)nn1] | 13% yield [M + H]⁺ = 807.2 ¹H-NMR (400 MHz, DMSO- d₆, VT): δ ppm 1.06-1.26 (m, 5H), 1.41-1.44 (m, 1H), 1.49- 1.59 (m, 2H), 1.63 (d, J = 6.8 Hz, 3H), 1.65-1.75 (m, 2H), 1.90-1.93 (m, 2H), 2.16-2.20 (m, 2H), 2.73-2.85 (m, 3H), 3.30-3.40 (m, 2H), 3.52-3.78 (m, 3H), 3.87-3.92 (m, 4H), 4.90-4.98 (m, 1H), 6.87-6.93 (m, 2H), 7.08 (d, J = 8.0 Hz, 1H), 7.53-7.55 (m, 1H), 7.71 (s, 1H), 8.30 (s, 1H), 8.43 (s, 1H), 8.65 (s, 1H), 8.96 (s, 1H), 9.00 (s, 1H), 10.21 (s, 1H). Five protons are not apparent |
| P-499 | A-57 | C-118 | 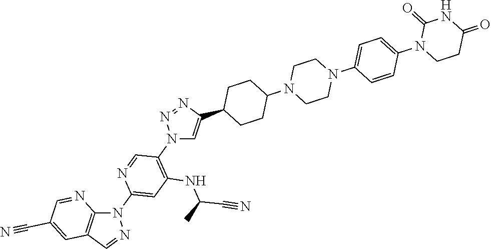 | 12.7% yield [M + H]⁺ = 711.5 ¹H-NMR (400 MHz, DMSO- d₆): δ 1.44-1.55 (m, 4H), 1.61 (d, J = 6.8 Hz, 3H), 1.96-2.01 (m, 2H), 2.16-2.19 (m, 2H), 2.37-2.42 (m, 1H), 2.66-2.87 (m, 7H), 3.10-3.19 (m, 4H), 3.70 (t, J = 6.8 Hz, 2H), 4.97- 5.05 (m, 1H), 6.93 (d, J = 9.2 Hz, 2H), 7.14-7.20 (m, 3H), 7.67 (s, 1H), 8.36-8.41 (m, 2H), 8.71 (s, 1H), 9.05-9.07 (m, 2H), 10.25 (s, 1H) |
| P-500 | A-104 | C-187b | 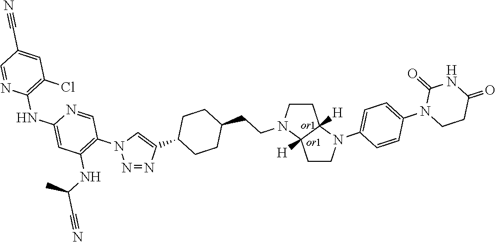 [SMILES: C[C@H](C#N)Nc1cc(Nc2ncc(C#N)cc2Cl)ncc1-n1cc([C@H]2CC[C@H](CCN3CC[C@H]4[C@@H]3CCN4c3ccc(N4CCC(=O)NC4=O)cc3)CC2)nn1] | 21.1% yield [M + H]⁺ = 774.2 ¹H-NMR (400 MHz, DMSO- d₆): δ ppm 1.17-1.20 (m, 2H), 1.47-1.60 (m, 3H), 1.61-1.67 (m, 6H), 1.87-1.90 (m, 2H), 2.13-2.16 (m, 2H), 2.33-2.34 (m, 2H), 2.67-2.69 (m, 4H), 3.23-3.30 (m, 3H), 3.67-3.71 (m, 3H), 4.12-4.31 (m, 1H), 4.45-4.59 (m, 1H), 4.81-4.84 (m, 1H), 6.68 (d, J = 9.20 Hz, 2H), 6.97 (d, J = 7.60 Hz, 1H), 7.16-7.23 (m, 2H), 7.74 (s, 1H), 8.17 (s, 1H), 8.28 (s, |
| | | | | 1H), 8.49 (s, 1H), 8.64 (s, |
| | | | | 1H), 9.30 (s, 1H), 10.25 (s, |
| | | | | 1H). |
| | | | | Two protons are not apparent |
| P-502 | A-57 | C-184 | 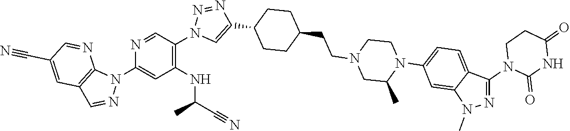 [SMILES: C[C@H](C#N)Nc1cc(-n2ncc3cc(C#N)cnc32)ncc1-n1cc([C@H]2CC[C@H](CCN3CCN(c4ccc5c(N6CCC(=O)NC6=O)nn(C)c5c4)[C@@H](C)C3)CC2)nn1] | 6.8% yield [M + H]⁺ = 807 ¹H-NMR (400 MHz, DMSO- d₆): δ ppm 0.98-1.02 (m, 1H), 1.09-1.15 (m, 2H), 1.15-1.29 (m, 2H), 1.38-1.48 (m, 1H), 1.49-1.60 (m, 2H), 1.62 (d, J = 6.8 Hz, 3H), 1.63-1.80 (m, 2H), 1.88-1.97 (m, 2H), 2.16- |
| | | | | 2.22 (m, 2H), 2.72-2.86 (m, |
| | | | | 3H), 3.12-3.38 (m, 6H), 3.65- |
| | | | | 3.76 (m, 2H), 3.88-3.98 (m, |
| | | | | 5H), 4.49-4.58 (m, 1H), 5.00- |
| | | | | 5.09 (m, 1H), 6.89-7.00 (m, |
| | | | | 2H), 7.18-7.24 (m, 1H), 7.51- |
| | | | | 7.57 (m, 1H), 7.68 (s, 1H), |
| | | | | 8.38 (s, 1H), 8.42 (s, 1H), |
| | | | | 8.73 (s, 1H), 9.05-9.10 (m, |
| | | | | 2H), 10.52 (s, 1H) |
| P-503 | A-104 | C-180a | 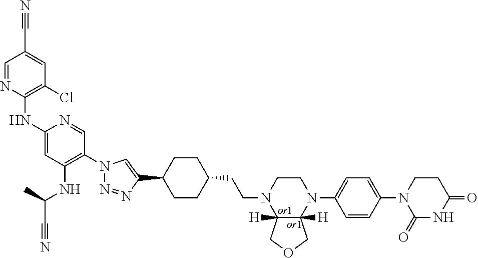 [SMILES: C[C@H](C#N)Nc1cc(Nc2ncc(C#N)cc2Cl)ncc1-n1cc([C@H]2CC[C@H](CCN3CCN(c4ccc(N5CCC(=O)NC5=O)cc4)[C@@H]4COC[C@@H]43)CC2)nn1] | 38.5% yield [M + H]⁺ = 790.2 ¹H-NMR (400 MHz, DMSO- d₆): δ ppm 1.19-1.22 (m, 2H), 1.43-1.50 (m, 3H), 1.61 (d, J = 7.20 Hz, 3H), 1.62-1.80 (m, 2H), 1.86-1.89 (m, 2H), 2.13-2.16 (m, 2H), 2.67-2.75 (m, 3H), 3.19-3.22 (m, 3H), 3.40-3.53 (m, 1H), 3.61-3.73 (m, 3H), 3.72-3.83 (m, 1H), 3.87-3.98 (m, 3H), 4.32-4.35 (m, 2H), 4.81-4.85 (m, 1H) 4.99-5.01 (s, 1H), 6.98 (d, J = 7.60 Hz, 1H), 7.01 (d, J = 8.80 Hz, 2H), 7.22 (d, J = |
| | | | | 8.80 Hz, 2H), 7.74 (s, 1H), |
| | | | | 8.17 (s, 1H), 8.28 (s, 1H), |
| | | | | 8.49 (d, J = 2.00 Hz, 1H), |
| | | | | 8.65 (d, J = 2.00 Hz, 1H), |
| | | | | 9.30 (s, 1H), 10.29 (s, 1H). |
| P-504 | A-57 | C-180b | 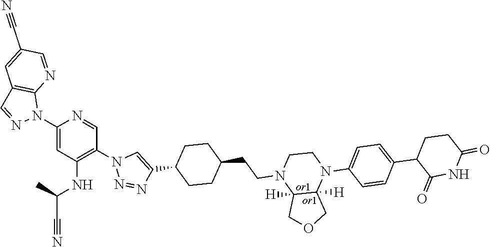 [SMILES: C[C@H](C#N)Nc1cc(-n2ncc3cc(C#N)cnc32)ncc1-n1cc([C@H]2CC[C@H](CCN3CCN(c4ccc(C5CCC(=O)NC5=O)cc4)[C@H]4COC[C@H]43)CC2)nn1] | 21.5% yield [M + H]⁺ = 781.2 ¹H-NMR (400 MHz, DMSO- d₆): δ ppm 1.21-1.24 (m, 2H), 1.50-1.53 (m, 4H), 1.62 (d, J = 7.20 Hz, 3H), 1.64-1.66 (m, 2H), 1.88-1.91 (m, 2H), 2.16-2.19 (m, 2H), 2.67-2.72 (m, 2H), 2.71-2.78 (m, 1H), 3.19-3.22 (m, 3H), 3.61-3.69 (m, 3H), 3.71-3.73 (m, 2H), 3.92-3.98 (m, 3H), 4.32-4.35 (m, 1H), 5.01-5.05 (m, 2H), 7.02-7.08 (m, 2H), 7.18-7.26 (m, 3H), 7.68 (s, 1H), 8.38 |
| | | | | (s, 1H), 8.42 (s, 1H), 8.73 (s, |
| | | | | 1H), 9.06-9.08 (m, 2H), |
| | | | | 10.29 (s, 1H) |
| P-505 | A-57 | C-159b | 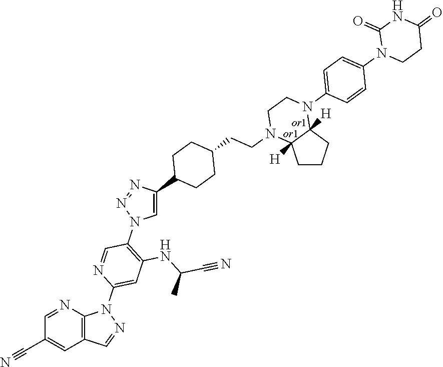 | 14.2% yield [M + H]⁺ = 779.2 ¹H-NMR (400 MHz, DMSO- d₆): δ ppm 1.16-1.26 (m, 2H), 1.39-1.54 (m, 3H), 1.62 (d, J = 7.2 Hz, 3H), 1.64-1.85 (m, 5H), 1.89-1.92 (m, 2H), 2.00-2.19 (m, 4H), 2.67-2.83 (m, 3H), 3.05-3.19 (m, 3H), 3.58-3.80 (m, 5H), 4.38-4.46 (m, 1H), 4.99-5.06 (m, 1H), 6.98-7.29 (m, 5H), 7.68 (s, 1H), 8.37 (s, 1H), 8.42 (s, 1H), 8.73 (s, 1H), 9.06-9.08 (m, 2H), 10.28 and 10.34 (s, 1H). Two protons are not apparent |
| P-506 | A-104 | C-159b | 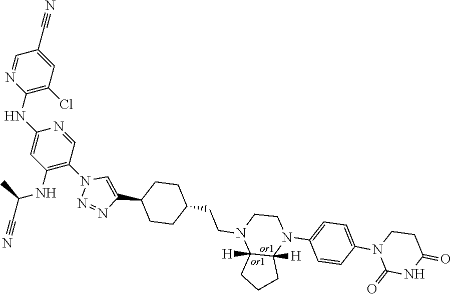 [SMILES: C[C@H](C#N)Nc1cc(Nc2ncc(C#N)cc2Cl)ncc1-n1cc([C@H]2CC[C@H](CCN3CCN(c4ccc(N5CCC(=O)NC5=O)cc4)[C@@H]4CCC[C@@H]43)CC2)nn1] | 59.6% yield [M + H]⁺ = 788.2 ¹H-NMR (400 MHz, DMSO- d₆): δ ppm 1.12-1.29 (m, 2H), 1.37-1.52 (m, 3H), 1.60 (d, J = 6.8 Hz, 3H), 1.62-1.81 (m, 5H), 1.87-1.90 (m, 2H), 1.98- 2.16 (m, 4H), 2.67-2.80 (m, 3H), 3.08-3.20 (m, 3H), 3.35- 3.49 (m, 2H), 4.39-4.49 (m, 1H), 4.79-4.88 (m, 1H), 6.96- 7.29 (m, 5H), 7.73 (m, 1H), 8.16 (s, 1H), 8.27 (s, 1H), 8.47-8.48 (m, 1H), 8.63-8.64 (m, 1H), 9.29 (s, 1H), 10.27 and 10.33 (s, 1H). Five protons are not apparent |
| P-507 | A-107 | C-187b | 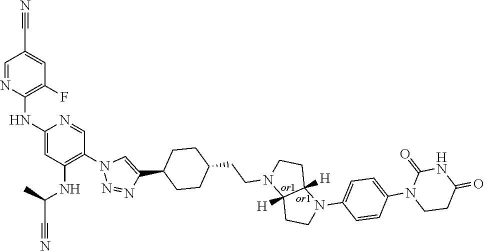 [SMILES: C[C@H](C#N)Nc1cc(Nc2ncc(C#N)cc2F)ncc1-n1cc([C@H]2CC[C@H](CCN3CC[C@H]4[C@@H]3CCN4c3ccc(N4CCC(=O)NC4=O)cc3)CC2)nn1] | 8.2% yield [M + H]⁺ = 758.2 ¹H-NMR (400 MHz, DMSO- d₆): δ ppm 1.17-1.19 (m, 2H), 1.41-1.52 (m, 3H), 1.60 (d, J = 6.8 Hz, 3H), 1.63-1.67 (m, 2H), 1.87-1.90 (m, 2H), 2.13- 2.16 (m, 2H), 2.67-2.69 (m, 2H), 3.21-3.52 (m, 4H), 3.67- 3.69 (m, 2H), 4.28-4.33 (m, 1H), 4.41-4.56 (m, 1H), 4.77- 4.80 (m, 1H), 6.69 (d, J = 6.80 Hz, 2H), 6.82 (d, J = 8.00 Hz, 1H), 7.16-7.20 (m, 2H), 7.74 (s, 1H), 8.12 (s, 1H), 8.21 (d, J = 1.6 Hz, 1H), |
| | | | | 8.24 (s, 1H), 8.53 (s, 1H), |
| | | | | 10.06 (s, 1H), 10.25 (s, 1H). |
| | | | | Seven protons are not |
| | | | | apparent |
| | | | | ¹⁹F-NMR (376 MHz, DMSO- |
| | | | | d₆): δ ppm −130.909 |
| P-508 | A-57 | C-180a | 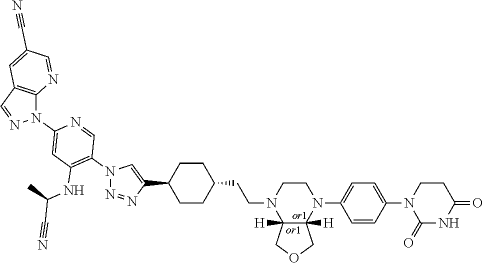 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)cc4)[C@@H]4COC[C@@H]43)CC2)nn1] | 22.2% yield [M + H]⁺ = 781 ¹H-NMR (400 MHz, DMSO- d₆): δ ppm 1.14-1.25 (m, 2H), 1.39-1.58 (m, 3H), 1.62 (d, J = 6.8 Hz, 3H), 1.65-1.80 (m, 2H), 1.85-1.95 (m, 2H), 2.15- 2.19 (m, 2H), 2.68-2.81 (m, 3H), 3.20-3.22 (m, 3H), 3.45- 3.98 (m, 7H), 4.32-4.38 (m, 1H), 4.98-5.09 (m, 2H), 7.03 (d, J = 9.2 Hz, 2H), 7.18-7.23 (m, 3H), 7.68 (m, 1H), 8.38 (s, 1H), 8.42 (s, 1H), 8.73 (s, 1H), 9.06-9.09 (m, 2H), 10.29 (s, 1H). Two protons are not apparent |
| P-509 | A-104 | C-159a | 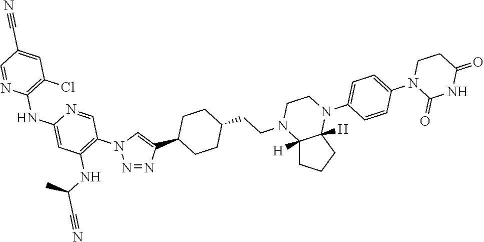 [SMILES: C[C@H](C#N)Nc1cc(Nc2ncc(C#N)cc2Cl)ncc1-n1cc([C@H]2CC[C@H](CCN3CCN(c4ccc(N5CCC(=O)NC5=O)cc4)[C@@H]4CCC[C@@H]43)CC2)nn1] | 51.1% yield [M + H]⁺ = 788 ¹H-NMR (400 MHz, DMSO- d₆): δ ppm 1.15-1.28 (m, 2H), 1.37-1.54 (m, 3H), 1.61 (d, J = 6.8 Hz, 3H), 1.62-1.84 (m, 5H), 1.86-1.92 (m, 2H), 1.98- 2.06 (m, 1H), 2.10-2.20 (m, 3H), 2.68-2.81 (m, 3H), 3.10- 3.23 (m, 3H), 3.35-3.45 (m, 1H), 3.59-3.80 (m, 5H), 4.39- 4.47 (m, 1H), 4.78-4.88 (m, 1H), 6.93-7.01 (m, 2H), 7.09- 7.31 (m, 2H), 7.74 (s, 1H), 8.16 (s, 1H), 8.28 (s, 1H), |
| | | | | 8.48 (s, 1H), 8.63-8.64 (m, |
| | | | | 1H), 8.99 (s, 1H), 9.25 (s, |
| | | | | 1H), 10.28 and 10.34 (s, 1H). |
| | | | | One proton is not apparent |
| P-510 | A-104 | C-187a | 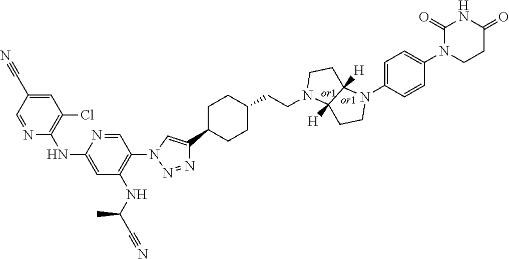 [SMILES: C[C@H](C#N)Nc1cc(Nc2ncc(C#N)cc2Cl)ncc1-n1cc([C@H]2CC[C@H](CCN3CC[C@H]4[C@@H]3CCN4c3ccc(N4CCC(=O)NC4=O)cc3)CC2)nn1] | 55.3% yield [M + H]⁺ = 774 ¹H-NMR (400 MHz, DMSO- d₆): δ ppm 1.11-1.26 (m, 2H), 1.35-1.54 (m, 3H), 1.59 (d, J = 6.8 Hz, 3H), 1.61-1.72 (m, 3H), 1.85-1.92 (m, 2H), 2.13- 2.16 (m, 2H), 2.29-2.40 (m, 1H), 2.66-2.80 (m, 4H), 3.16- 3.32 (m, 3H), 3.35-3.45 (m, 2H), 3.52-3.60 (m, 1H), 3.68 (t, J = 6.8 Hz, 2H), 4.23-4.32 (m, 1H), 4.45-4.52 (m, 1H), 4.78-4.85 (m, 1H), 6.67 (d, J = 8.8 Hz, 2H), 6.91-6.95 (m, 1H), 7.15-7.19 (m, 2H), |
| | | | | 7.74 (s, 1H), 8.15 (s, 1H), |
| | | | | 8.27 (s, 1H), 8.47-8.48 (m, |
| | | | | 1H), 8.63-8.64 (m, 1H), 9.23 |
| | | | | (s, 1H), 10.24 (s, 1H) |
| | | | | One proton is not apparent |
| P-511 | A-57 | C-159a | 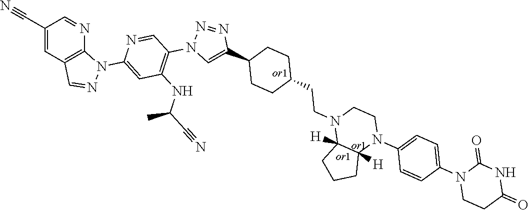 [SMILES: C[C@H](C#N)Nc1cc(-n2ncc3cc(C#N)cnc32)ncc1-n1cc([C@H]2CC[C@H](CCN3CCN(c4ccc(N5CCC(=O)NC5=O)cc4)[C@@H]4CCC[C@@H]43)CC2)nn1] | 23.2% yield [M + H]⁺ = 779.1 ¹H-NMR (400 MHz, DMSO- d₆): δ ppm 1.15-1.30 (m, 2H), 1.39-1.60 (m, 3H), 1.61 (d, J = 6.8 Hz, 3H), 1.62-1.88 (m, 5H), 1.89-1.93 (m, 2H), 1.97- 2.25 (m, 4H), 2.67-2.72 (m, 2H), 2.75-2.82 (m, 1H), 3.10- 3.23 (m, 3H), 3.32-3.42 (m, 1H), 3.58-3.70 (m, 3H), 4.39- 4.45 (m, 1H), 4.98-5.06 (m, 1H), 6.98-7.30 (m, 5H), 7.68 |
| | | | | (s, 1H), 8.38 (s, 1H), 8.42 (s, |
| | | | | 1H), 8.73 (s, 1H), 9.06-9.08 |
| | | | | (m, 2H), 10.28 and 10.34 (s, |
| | | | | 1H). |
| | | | | Three protons are not |
| | | | | apparent |
| P-512 | A-104 | C-180b | 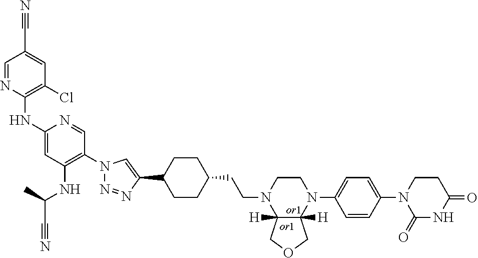 | 23.3% yield [M + H]⁺ = 790.5 ¹H-NMR (400 MHz, DMSO- d₆): δ ppm 1.11-1.29 (m, 2H), 1.39-1.53 (m, 3H), 1.60 (d, J = 6.8 Hz, 3H), 1.61-1.80 (m, 2H), 1.84-1.91 (m, 2H), 2.12-2.16 (m, 2H), 2.67-2.74 (m, 3H), 3.15-3.25 (m, 3H), 3.40-3.51 (m, 1H), 3.59-3.75 (m, 4H), 3.75-3.82 (m, 1H), 3.88-4.03 (m, 3H), 4.31-4.35 (m, 1H), 4.78-4.90 (m, 1H), 4.98-5.04 (m, 1H), 6.98-7.04 (m, 3H), 7.20-7.23 (m, 2H), 7.73 (s, 1H), 8.17 (s, 1H), |
| | | | | 8.28 (s, 1H), 8.48-8.50 (m, |
| | | | | 1H), 8.63-8.64 (m, 1H), 9.32 |
| | | | | (s, 1H), 10.28 (s, 1H). |

Table 72 summarizes the final compounds that were prepared using General Procedure X-8.

TABLE 72
Final Compounds Prepared using General Procedure X-8

| Cmpd | | | | |
|---|---|---|---|---|
| No. | TBM | CBM | Structure | Characterization |
| P-321 | A-29 | C-118 | 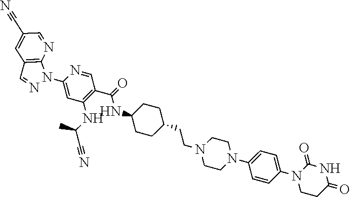 [SMILES: C[C@H](C#N)Nc1cc(-n2ncc3cc(C#N)cnc32)ncc1C(=O)N[C@H]1CC[C@H](CCN2CCN(c3ccc(N4CCC(=O)NC4=O)cc3)CC2)CC1] | 58.2% yield [M + H]⁺ = 715.2 ¹H-NMR (400 MHz, DMSO-d₆): δ ppm 1.02 − 1.18 (m, 2H), 1.22 − 1.48 (m, 3H), 1.60 − 1.66 (m, 2H), 1.68 (d, J = 7.2 Hz, 3H), 1.81 − 1.94 (m, 4H), 2.68 − 2.70 (m, 2H), 2.90 − 3.03 (m, 2H), 3.10 − 3.30 (m, 4H), 3.55 − 3.72 (m, 4H), 3.82 − 3.90 (m, 3H), 4.91 − 5.04 (m, 1H), 7.04 (d, J = 8.8 Hz, 2H), 7.23 (d, J = 8.8 Hz, 2H), 7.50 (s, 1H), 8.56 − 8.58 (m, 1H), 8.71 − 8.73 (m, 2H), 8.76 − 8.78 (m, 1H), 9.05 − 9.06 (m, 2H), 10.29 (s, 1H). |
| P-433 | A-29 | C-147 | 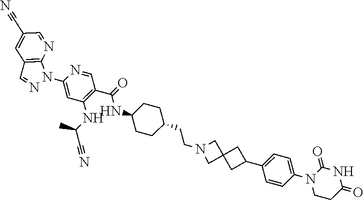 [SMILES: C[C@H](C#N)Nc1cc(-n2ncc3cc(C#N)cnc32)ncc1C(=O)N[C@H]1CC[C@H](CCN2CC3(CC(c4ccc(N5CCC(=O)NC5=O)cc4)C3)C2)CC1] | 17.5% yield [M + H]⁺ = 726.3 ¹H-NMR (400 MHz, DMSO d6): δ ppm 0.99 − 1.09 (m, 2H), 1.28 − 1.39 (m, 5H), 1.68 (d, J = 6.8 Hz, 3H), 1.76 − 1.79 (m, 2H), 1.92 (brs, 2H), 2.25 − 2.39 (m, 2H), 2.53 − 2.60 (m, 1H), 2.67 − 2.72 (m, 3H), 3.16 − 3.18 (m, 2H), 3.35 − 3.44 (m, 1H), 3.74 − 3.78 (m, 3H), 3.94 − 3.98 (m, 1H), 4.05 − 4.12 (m, 2H), 4.30 − 4.35 (m, 1H), 4.00- 4.99 (m, 1H), 7.23 − 7.28 (m, 4H), 7.51 (s, 1H), 8.57 (d, J = 8.0 Hz, 1H), 8.70 (s, 1H), 8.72 (s, 1H), 8.77 (d, J = 7.6 Hz, 1H), 9.04 − 9.05 (m, 2H), 10.33 (s, 1H). |
| P-475 | A-29 | C-185b | 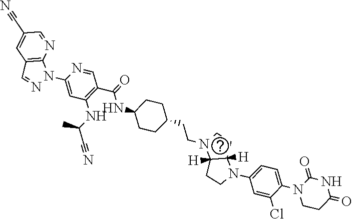 [SMILES: C[C@H](C#N)Nc1cc(-n2ncc3cc(C#N)cnc32)ncc1C(=O)N[C@H]1CC[C@H](CCN2CC[C@H]3[C@@H]2CCN3c2ccc(N3CCC(=O)NC3=O)c(Cl)c2)CC1] | 3.4% yield [M + H]⁺ = 775.2 1H-NMR (400 MHz, DMSO-d₆): δ ppm 1.09 − 1.13 (m, 2H), 1.34 − 1.43 (m, 3H), 1.62 − 1.69 (m, 5H), 1.77 − 1.84 (m, 2H), 1.91 − 1.98 (m, 2H), 2.71 − 2.74 (m, 3H), 3.20 − 3.26 (m, 2H), 3.60 − 3.66 (m, 2H), 3.76 − 3.81 (m, 1H), 4.22 − 4.28 (s, 1H), 4.53 − 4.55 (m, 1H), 4.94 − 4.98 (m, 1H), 6.67 (d, J = 9.2 Hz, 1H), 6.78 (s, 1H), 7.29 (d, J = 6.0 Hz, 1H), 7.50 (s, 1H) , 8.57 (d, J = 8.0 Hz, 1H) , 8.71 − 8.78 (m, 3H), 9.05 − 9.06 (m, 2H), 10.36 (s, 1H) Seven protons are not apparent |
| P-481 | A-29 | C-181b | 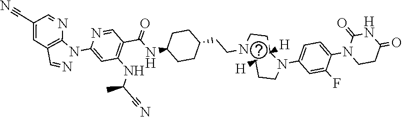 [SMILES: C[C@H](C#N)Nc1cc(-n2ncc3cc(C#N)cnc32)ncc1C(=O)N[C@H]1CC[C@H](CCN2CC[C@H]3[C@@H]2CCN3c2ccc(N3CCC(=O)NC3=O)c(F)c2)CC1] | 43.8% yield [M + H]⁺ = 759.2 ¹H-NMR (400 MHz, DMSO-d₆): δ ppm 1.05 − 1.16 (m, 2H), 1.36 − 1.42 (m, 3H), 1.63 − 1.68 (m, 5H), 1.81 − 1.86 (m, 2H), 1.94 − 1.98 (m, 2H), 2.31 − 2.33 (m, 2H), 2.66 − 2.71 (m, 3H), 3.18 − 3.30 (m, 3H), 3.35 − 3.52 (m, 3H), 3.62 (t, J = 6.8 Hz, 2H), 4.25 − 4.32 (m, 1H), 4.49 − |
| | | | | 4.55 (m, 1H), 4.89 − 4.98 (m, 1H), 6.48 − 6.59 |
| | | | | (m, 2H), 7.20 − 7.25 (m, 1H), 7.49 (s, 1H), |
| | | | | 8.56 (d, J = 7.6 Hz, 1H), 8.70 − 8.77 (m, 3H), |
| | | | | 9.04 − 9.05 (m, 2H), 10.36 (s, 1H). |
| | | | | Four protons are not apparent |
| | | | | ¹⁹F-NMR (376 MHz, DMSO-d₆): δ |
| | | | | ppm −120.395 |
| P-484 | A-29 | C-181a | 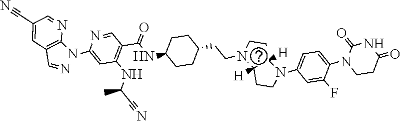 [SMILES: C[C@H](C#N)Nc1cc(-n2ncc3cc(C#N)cnc32)ncc1C(=O)N[C@H]1CC[C@H](CCN2CC[C@H]3[C@@H]2CCN3c2ccc(N3CCC(=O)NC3=O)c(F)c2)CC1] | 61% yield [M + H]⁺ = 759.1 ¹H-NMR (400 MHz, DMSO-d₆): δ ppm 1.06 − 1.18 (m, 2H), 1.33 − 1.42 (m, 3H), 1.61 − 1.66 (m, 2H), 1.67 (d, J = 6.80 Hz, 3H), 1.81 − 1.84 (m, 2H), 1.93 (m, 2H), 2.32 − 2.34 (m, 2H), 2.66 − 2.71 (m, 2H), 3.10 − 3.18 (m, 2H), 3.32 − 3.44 (m, 2H), 4.21 − 4.32 (m, 1H), |
| | | | | 4.49 − 4.60 (m, 1H), 4.93 − 4.99 (m, 1H), |
| | | | | 6.51 − 6.59 (m, 2H), 7.20 − 7.26 (m, 1H), |
| | | | | 7.49 (s, 1H), 8.56 (d, J = 7.60 Hz, 1H), 8.70 (s, |
| | | | | 1H), 8.72 (s, 1H), 8.76 (d, J = 7.60 Hz, 1H), |
| | | | | 9.04 − 9.05 (m, 2H), 10.36 (s, 1H) |
| | | | | Seven protons are not apparent |
| | | | | ¹⁹F-NMR (376 MHz, DMSO-d₆): δ |
| | | | | ppm −120.396 ppm |
| P-488 | A-29 | C-185a | 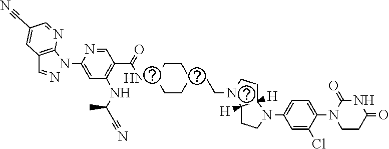 | 20.6% yield [M + H]⁺ = 775.2 1H-NMR (400 MHz, DMSO-d₆): δ 1.09 − 1.16 (m, 2H), 1.34 − 1.40 (m, 3H), 1.62 − 1.69 (m, 6H), 1.80 − 1.83 (m, 2H), 1.90 − 2.07 (m, 2H), 2.25 − 2.27 (m, 3H), 2.71 (t, J = 6.8 Hz, 2H), 3.18 − 3.29 (m, 2H), 3.35 − 3.40 (m, 2H), 3.51 − 3.59 (m, 1H), 3.71 − 3.79 (m, 4H), 4.14 − 4.25 (m, 1H), 4.81 − 4.91 (m, 1H), 4.93 − 4.97 (m, 1H), 7.12 − |
| | | | | 7.15 (m, 1H), 7.24 − 7.27 (m, 1H), 7.43 (m, |
| | | | | 1H), 7.50 (s, 1H), 8.58 (d, J = 7.6 Hz, 1H), |
| | | | | 8.70 − 8.72 (m, 2H), 8.76 − 8.78 (m, 1H), |
| | | | | 9.03 − 9.05 (m, 2H), 10.38 (s, 1H) |
| P-489 | A-29 | C-186a | 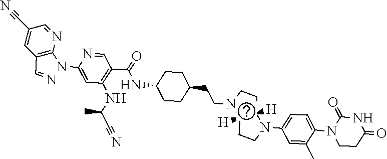 [SMILES: Cc1cc(N2CC[C@H]3[C@@H]2CCN3CC[C@H]2CC[C@H](NC(=O)c3cnc(-n4ncc5cc(C#N)cnc54)cc3N[C@H](C)C#N)CC2)ccc1N1CCC(=O)NC1=O] | 10% yield [M + H]⁺ = 755.2 ¹H-NMR (400 MHz, DMSO-d₆): δ ppm 1.07 − 1.15 (m, 2H),1.33 − 1.41 (m, 3H), 1.50 − 1.62 (m, 3H), 1.67 (d, J = 7.2 Hz, 3H), 1.81 − 1.84 (m, 2H), 1.91 − 1.97 (m, 2H), 2.11 − 2.18 (m, 2H), 2.25 − 2.28 (m, 3H), 2.68 − 2.70 (m, 2H), 2.84 − 2.90 (m, 1H), 3.17 − 3.22 (m, 2H), 3.51 − 3.62 (m, 2H), 3.73 (t, J = 6.8 Hz, 2H), 3.77 − 3.83 |
| | | | | (m, 1H), 4.12 − 4.18 (m, 1H), 4.40 − 4.43 |
| | | | | (m, 1H), 4.92 − 4.99 (m, 1H), 7.00 − 7.02 |
| | | | | (m, 1H), 7.07 − 7.15 (m, 2H), 7.49 (s, 1H), |
| | | | | 8.56 (d, J = 7.6 Hz, 1H), 8.70 (s, 1H), 8.72 |
| | | | | (s, 1H), 8.76 (d, J = 7.6 Hz, 1H), 9.03 − |
| | | | | 9.05 (m, 2H), 10.31 (s, 1H). |
| | | | | Two protons are not apparent |
| P-490 | A-29 | C-186b | 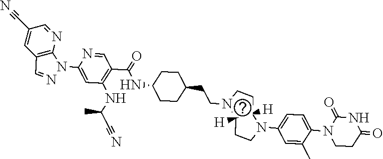 [SMILES: Cc1cc(N2CC[C@H]3[C@@H]2CCN3CC[C@H]2CC[C@H](NC(=O)c3cnc(-n4ncc5cc(C#N)cnc54)cc3N[C@H](C)C#N)CC2)ccc1N1CCC(=O)NC1=O] | 20.3% yield [M + H]+ = 755.2 1H-NMR (400 MHz, DMSO-d6): δ ppm 1.05 − 1.14 (m, 2H), 1.32 − 1.42 (m, 3H), 1.50 − 1.62 (m, 2H), 1.67 (d, J = 6.8 Hz, 3H), 1.81 − 1.84 (m, 2H), 1.91 − 1.96 (m, 2H), 2.11 − 2.19 (m, 2H), 2.25 − 2.27 (m, 3H), 2.67 − 2.70 (m, 2H), 2.90 − 2.93 (m, 1H), 3.13 − 3.21 (m, 2H), 3.29 − 3.33 (m, 1H), 3.52 − 3.65 (m, 2H), 3.71 − 3.74 (m, |
| | | | | 2H), 3.77 − 3.80 (m, 1H), 4.13 − 4.18 (m, |
| | | | | 2H), 4.40 − 4.43 (m, 2H), 4.91 − 4.98 (m, |
| | | | | 1H), 7.00 − 7.02 (m, 1H), 7.08 − 7.14 (m, |
| | | | | 2H), 7.49 (s, 1H), 8.56 (d, J = 8.0 Hz, 1H), |
| | | | | 8.70 (s, 1H), 8.72 (s, 1H), 8.76 (d, J = 7.6 |
| | | | | Hz, 1H), 9.05 − 9.04 (m, 2H), 10.30 (s, 1H). |

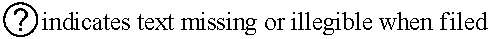

Table 73 summarizes the final compounds that were prepared using General Procedure X-9.

TABLE 73
Final Compounds Prepared using General Procedure X-9

| Cmpd | | | | |
|---|---|---|---|---|
| No. | TBM | CBM | Structure | Characterization |
| P-501 | A-77 | C-128 | 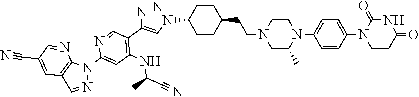 [SMILES: C[C@H](C#N)Nc1cc(-n2ncc3cc(C#N)cnc32)ncc1-c1cn([C@H]2CC[C@H](CCN3CCN(c4ccc(N5CCC(=O)NC5=O)cc4)[C@H](C)C3)CC2)nn1] | 22.4% yield [M + H]⁺ = 53.4 ¹H-NMR (400 MHz, DMSO-d₆): δ ppm 0.90 − 1.12 (m, 3H), 1.20 − 1.35 (m, 2H), 1.40 − 1.51 (m, 1H), 1.61 − 1.72 (m, 2H), 1.73 (d, J = 7.2 Hz, 3H), 1.82-1.98 (m, 4H), 2.19 − 2.28 (m, 2H), 2.66-2.73 (m, 3H), 3.00 − 3.30 (m, 5H), 3.48 − |
| | | | | 3.67 (m, 3H), 3.68 − 3.72 (m, 1H), 4.30 − 4.40 |
| | | | | (m, 1H), 4.55 − 4.65 (m, 1H), 4.98 − 5.04 (m, |
| | | | | 1H), 6.91 − 7.00 (m, 2H), 7.16 − 7.33 (m, 2H), |
| | | | | 7.55 (s, 1H), 8.46 − 8.52 (m, 1H), 8.65 (s, 1H), |
| | | | | 8.71 (s, 1H), 8.85 (s, 1H), 8.97 − 9.01 (m, 2H), |
| | | | | 9.87 (s, 1H) |

### Biological Examples

### Example B1. Reagent Preparations

Cell culture media was prepared in a tissue culture hood in a sterile environment by adding 10% FBS and 1% Penicillin Streptomycin to 500 mL no phenol red RPMI 1640 media. The media was filtered through a Nalgene Bottle Top Filter and stored at 4° C.

The Cell titer Glo (CTG) buffer and substrate (CellTiter-Glo Luminescent Cell Viability Assay, Promega Ref. #G7573) were stored in −20° C. The CTG buffer (100 mL) was warmed in a bead bath and added to the CTG substrate bottle in a tissue culture hood. The solution was mixed with a pipette until it became homogenous. CTG reagent were aliquoted into 15 mL falcon tubes and stored at −20° C.

For Homogenous Time Resolved Fluorescence (HTRF) assays, a Cisbio HTRF kit was used, which included: Lysis Buffer #1 4×, Blocking Reagent #3 100×, 20× Antibody 1 (Anti-IRAK4 d2), 20× Antibody 2 (Anti-IRAK4 k), and Detection Buffer.

4× Lysis Buffer was stored at 4° C. For use as 1× Lysis buffer, the 4× solution was diluted with de-ionized water (distilled water, Gibco Cat. #15230279) and 100× Blocking Reagent in a 1:3:0.04 volume ratio.

20× Antibody Solution aliquots were stored in −80° C. and the Detection Buffer was stored in 4° C. For use as a 1× Antibody Solution, the 20× Antibody Solution aliquot was diluted with Detection Buffer in a 1:19 volume ratio.

### Example B2. Advanced Lipoxidation End Product THP1 Homogeneous Time Resolved Fluorescence (ALE THP1 HTRF) Procedure

Cells were lysed at room temperature for 45 min with shaking. A BCA protein assay was performed and normalization was conducted to the desired total protein concentration with 1× lysis buffer. Next, 1× Antibody Solutions were prepared by adding 380 μL Detection Buffer to 20 μL 20× Antibody Solution aliquots and mixing well. 1× Antibody Solutions were combined 1:1 and briefly vortexed. For control wells, 20 μL 1× anti IRAK4-k Antibody Solution was saved. The 384-well plate (ProxiPlate-384 Plus, Perkin Elmer Cat. #6008289) was loaded by adding 4 μL of the mixed Antibody Solution to empty wells using a single channel repeater. Using a multi-channel repeater, 16 μL lysate was added per well, and any bubbles that were formed were popped with 20 μL pipette tips and Kimwipe edges. In column 10, triplicates of each control was prepared. In wells A10, B10, and C10, buffer control was prepared by adding 16 μL Lysis Buffer and 4 μL Detection Buffer. In wells D10, E10, and F10, cryptate control was prepared by adding 16 μL Lysis Buffer, 2 μL Detection Buffer, and 2 μL 1× anti IRAK4-k Antibody Solution. In wells G10, H₁₀, and I10, a negative control was prepared by adding 16 μL Lysis Buffer and 4 μL mixed Antibody Solution. The plate was sealed with a clear seal and covered with an aluminum lid. The plate was spun down at 800 g for 5 min and incubated in the dark at room temperature overnight. The next day, the plate was spun down at 800 g for 5 min. The samples were analyzed by a plate reader (Envision, PerkinElmer) using the Desnor 384 HTRF program.

A summary of the ALE THP1 HTRF data for the tested compounds is provided in Table 48 below.

### Example B3. THP1 Homogeneous Time Resolved Fluorescence (LVL THP1 HTRF) Procedure

The THP1 plates were prepared by the following method. For each dosing plate, a duplicate for CTG assay was prepared. Two THP-1 T-175 flasks could be used to plate one 384 well plate. The cells were collected from two T-175 flasks into 200 mL centrifuge tubes and spun down at 1200 rpm for 10 min. The supernatant was removed completely and cells were resuspended in 7.5 mL cell culture media. To 5 μL of the cell suspension was added 25 μL of media and 30 μL of Trypan Blue stain. The cells were counted using countess (automated cell counter, Thermo Fisher Scientific) twice and the viability and live cell count was recorded. The actual cell count was 6× the live cell count. Based on the count, a cell suspension was prepared in colorless RPMI media for a final concentration of 7.5e6 cells/mL. Using Standard cassette multidrop combi, 20 μL of the 7.5e6 cells/mL cell stock was added to entire 384 well plate but for top half of column 1 (negative control) The multidrop combi was always primed to achieve a steady flow and washed after use with 20 mL de-ionized water followed by 20 mL alcohol. The cells were incubated in 5% CO₂ incubator at 37° C.

Dosing was conducted using the following method. To dose assay plates and CTG plate was added 20 nL of compounds in DMSO using Echo (Beckman Coulter), and the plates were incubated at 37° C. for 18 h. The compound plates were spun at 1200 rpm for 1 min before dosing. The compound plate was sealed and properly stored during incubation.

After dosing, an HTRF assay was conducted using the following method. The treatment plates (IRAK4_HTRF) were centrifuged at 600 g for 10 min. Using CyBio Felix, Automated liquid handler (Analytikjena), 13 μL cell culture media was removed. To all columns, 7.5 μL lysis buffer was added, and an additional 7.5 μL of lysis buffer was added to column 1(top half, negative control). The plate was incubated on a shaker at room temperature for 2 h. The plate was spun down at 600 g for 5 min. Custom 20×IRAK4 Antibody obtained from CISBIO Anti-IRAK4-d2 (acceptor Ab) and Anti-IRAK4-K (donor Ab) were diluted to 1× antibody mix Using a multichannel repeater, 4 μL of 1× antibody mix was added to each wellThe plate was spun down at 600 g for 5 min and incubated in the dark at room temperature for 18 h The next day (after 18 hrs), HTRF assay plate was spun down at 600 g for 5 min and read at 665/615 nm using a Envision plate reader.

Next, a CTG assay was run to assess cell death using the following method. To all wells 20 μL of CTG reagent was added, and the plate was covered The plate was placed on a shaker for 1-2 min and was let to sit at room temperature in the dark for 20 min (no more than 30 min). The plate was spun down at 1200 rpm for 1 min and the luminescence was analyzed using a plate reader (Envision, PerkinElmer).

A summary of the LVL THP1 HTRF data for the tested compounds is provided in Table 74 below.

TABLE 74
ALE THP1 HTRF and LVL THP1 HTRF
Results of the Compounds.

| | ALE THP1 | ALE THP1 | LVL THP1 | LVL THP1 |
|---|---|---|---|---|
| Compound | HTRF | HTRF | HTRF | HTRF |
| No. | EC₅₀ (μM) | Y_{MIN} (%) | EC₅₀ (μM) | Y_{MIN} (%) |
| P-1 | | | 0.148 | 33.3 |
| P-2 | 0.097 | 32.7 | | |
| P-3 | 0.273 | 35.6 | | |
| P-4 | 0.047 | 35.8 | | |
| P-5 | 0.054 | 17.1 | | |
| P-6 | 0.018 | 30.4 | | |
| P-7 | 0.037 | 30.8 | | |
| P-8 | 0.027 | 40.8 | | |
| P-9 | 0.025 | 20.0 | | |
| P-10 | 0.087 | 34.8 | | |
| P-11 | 0.001 | 47.5 | | |
| P-12 | 0.021 | 47.7 | | |
| P-13 | 0.082 | 43.4 | | |
| P-14 | 0.095 | 24.0 | | |
| P-15 | 0.026 | 31.8 | | |
| P-16 | 0.016 | 26.9 | | |
| P-17 | 0.013 | 24.6 | | |
| P-18 | 0.037 | 36.9 | | |
| P-19 | 0.017 | 38.0 | | |
| P-20 | 1.626 | 30.9 | | |
| P-21 | 0.022 | 40.3 | | |
| P-22 | 1.107 | 20.0 | | |
| P-23 | 0.020 | 34.8 | | |
| P-24 | 0.324 | 35.3 | | |
| P-25 | 0.012 | 20.1 | | |
| P-26 | 0.024 | 22.3 | | |
| P-27 | 0.040 | 28.4 | | |
| P-28 | 0.145 | 36.6 | | |
| P-29 | 0.098 | 14.1 | | |
| P-30 | 3.318 | 44.1 | | |
| P-31 | 0.060 | 18.2 | | |
| P-32 | 0.547 | 21.0 | | |
| P-33 | 0.008 | 15.9 | | |
| P-34 | 0.597 | 12.9 | | |
| P-35 | 0.041 | 8.4 | | |
| P-36 | 0.047 | 10.7 | | |
| P-37 | 0.132 | 20.7 | | |
| P-38 | 0.119 | 24.3 | | |
| P-39 | 0.198 | 24.3 | | |
| P-40 | 0.276 | 20.0 | | |
| P-41 | 0.110 | 41.6 | | |
| P-42 | 0.123 | 27.6 | | |
| P-43 | 0.312 | 14.7 | | |
| P-44 | 0.172 | 38.8 | | |
| P-45 | 0.028 | 11.4 | | |
| P-46 | 0.074 | 31.5 | | |
| P-47 | 0.110 | 14.1 | | |
| P-48 | 0.099 | 12.0 | | |
| P-49 | 0.021 | 10.9 | | |
| P-50 | 0.011 | 7.5 | | |
| P-51 | 0.009 | 7.9 | | |
| P-52 | 0.039 | 13.4 | | |
| P-53 | 1.887 | 37.6 | | |
| P-54 | 0.031 | 14.6 | | |
| P-55 | 0.015 | 10.3 | | |
| P-56 | 0.426 | 7.7 | | |
| P-57 | 0.194 | 9.7 | | |
| P-58 | 0.211 | 15.2 | | |
| P-59 | 0.107 | 8.5 | | |
| P-60 | 0.945 | 11.3 | | |
| P-61 | 0.101 | 9.2 | | |
| P-62 | 0.070 | 6.6 | | |
| P-63 | 0.006 | 8.3 | | |
| P-64 | 0.030 | 10.1 | | |
| P-65 | 0.010 | 7.3 | | |
| P-66 | 0.006 | 11.2 | | |
| P-67 | 0.034 | 9.3 | | |
| P-68 | 0.136 | 19.3 | | |
| P-69 | 0.417 | 15.4 | | |
| P-70 | 0.003 | 8.2 | | |
| P-71 | 0.008 | 10.9 | | |
| P-72 | 0.005 | 9.5 | | |
| P-73 | 0.058 | 8.1 | | |
| P-74 | 0.008 | 9.4 | | |
| P-75 | 0.062 | 11.4 | | |
| P-76 | 0.007 | 8.3 | | |
| P-77 | 0.028 | 11.3 | | |
| P-78 | 0.009 | 7.6 | | |
| P-79 | 0.043 | 12.2 | | |
| P-80 | 0.026 | 7.3 | | |
| P-81 | 0.073 | 10.7 | | |
| P-82 | 0.230 | 8.6 | | |
| P-83 | 0.047 | 8.1 | | |
| P-84 | 0.035 | 9.7 | | |
| P-85 | 0.117 | 11.7 | | |
| P-86 | 0.090 | 11.8 | | |
| P-87 | 0.026 | 10.5 | | |
| P-88 | 0.049 | 64.5 | | |
| P-89 | 0.023 | 10.3 | | |
| P-90 | 0.028 | 11.6 | | |
| P-91 | 0.005 | 7.0 | | |
| P-92 | 0.038 | 11.3 | | |
| P-93 | 0.023 | 13.0 | | |
| P-94 | 0.011 | 7.4 | | |
| P-95 | 0.015 | 9.9 | | |
| P-96 | 0.004 | 8.5 | | |
| P-97 | 0.088 | 15.1 | | |
| P-98 | 0.017 | 10.5 | | |
| P-99 | 0.022 | 10.7 | | |
| P-100 | 1.105 | 28.3 | | |
| P-101 | 0.092 | 24.6 | | |
| P-102 | 0.032 | 14.2 | | |
| P-103 | 0.029 | 16.2 | | |
| P-104 | 0.047 | 16.0 | | |
| P-105 | 0.076 | 11.5 | | |
| P-106 | 0.246 | 17.5 | | |
| P-107 | 0.014 | 9.1 | | |
| P-108 | 0.032 | 11.0 | | |
| P-109 | 0.008 | 10.7 | | |
| P-110 | 0.019 | 16.0 | | |
| P-111 | 0.024 | 15.6 | | |
| P-112 | 0.023 | 8.7 | | |
| P-113 | 0.074 | 18.7 | | |
| P-114 | >5.000 | 46.1 | | |
| P-115 | >5.000 | 38.9 | | |
| P-116 | 0.007 | 8.3 | | |
| P-117 | 0.033 | 13.6 | | |
| P-118 | 0.034 | 9.9 | | |
| P-119 | 0.025 | 8.3 | | |
| P-120 | 0.015 | 7.2 | | |
| P-121 | 0.013 | 11.0 | | |
| P-122 | 0.080 | 10.4 | | |
| P-123 | 0.062 | 24.4 | | |
| P-124 | 0.046 | 19.2 | | |
| P-125 | 0.010 | 9.8 | | |
| P-126 | 0.296 | 14.9 | | |
| P-127 | 0.008 | 24.5 | | |
| P-128 | 0.047 | 14.7 | | |
| P-129 | 0.047 | 24.2 | | |
| P-130 | 0.015 | 20.0 | | |
| P-131 | 0.003 | 5.3 | | |
| P-132 | 0.013 | 7.9 | | |
| P-133 | 0.010 | 6.9 | | |
| P-134 | 0.094 | 20.9 | | |
| P-135 | 0.001 | 7.3 | | |
| P-136 | 0.006 | 7.4 | | |
| P-137 | 0.018 | 7.7 | | |
| P-138 | 1.279 | 42.1 | | |
| P-139 | 0.001 | 5.6 | 0.011 | 1.7 |
| P-140 | 0.030 | 6.4 | | |
| P-141 | <0.001 | 6.1 | | |
| P-142 | 0.046 | 9.7 | | |
| P-143 | 0.004 | 6.5 | | |
| P-144 | 0.004 | 5.9 | 0.013 | 0.8 |
| P-145 | 0.035 | 11.0 | | |
| P-146 | 0.001 | 8.0 | | |
| P-147 | 0.019 | 9.0 | | |
| P-148 | 0.016 | 11.0 | | |
| P-149 | 0.004 | 7.1 | | |
| P-150 | 0.007 | 6.2 | 0.025 | <0.5 |
| P-151 | 0.008 | 6.0 | | |
| P-152 | 0.020 | 15.8 | | |
| P-153 | 0.017 | 4.5 | | |
| P-154 | 0.022 | 10.0 | | |
| P-155 | 0.002 | 10.2 | 0.006 | 0.9 |
| P-156 | 0.001 | 10.1 | 0.008 | 2.0 |
| P-157 | 0.002 | 10.0 | | |
| P-158 | 0.065 | 16.0 | | |
| P-159 | 0.007 | 6.1 | 0.018 | <0.5 |
| P-160 | 0.004 | 5.8 | 0.004 | 0.7 |
| P-161 | 0.006 | 6.6 | 0.012 | <0.5 |
| P-162 | 0.003 | 6.4 | 0.008 | 0.6 |
| P-163 | 0.012 | 8.4 | 0.026 | <0.5 |
| P-164 | 0.136 | 64.0 | >5.000 | 16.8 |
| P-165 | 0.002 | 5.4 | 0.006 | <0.5 |
| P-166 | 0.008 | 8.1 | 0.021 | <0.5 |
| P-167 | 0.002 | 5.9 | 0.005 | <0.5 |
| P-168 | 0.006 | 5.5 | 0.003 | <0.5 |
| P-169 | | | 0.026 | 3.0 |
| P-170 | | | 0.014 | 0.9 |
| P-171 | | | 0.023 | 1.2 |
| P-172 | | | 0.010 | 1.1 |
| P-173 | | | 0.011 | 0.7 |
| P-174 | 0.201 | 31.2 | 0.014 | 2.7 |
| P-175 | 0.003 | 7.2 | 0.003 | 0.9 |
| P-176 | 0.031 | 14.2 | 0.007 | <0.5 |
| P-177 | 0.004 | 6.7 | 0.032 | 6.4 |
| P-178 | 0.012 | 7.5 | 0.001 | 0.8 |
| P-179 | | | 0.065 | 2.0 |
| P-180 | 0.006 | 7.5 | 0.702 | 18.9 |
| P-181 | 0.168 | 27.7 | 0.112 | 18.3 |
| P-182 | 0.005 | 9.4 | 0.415 | 6.3 |
| P-183 | 0.063 | 27.9 | 0.012 | <0.5 |
| P-184 | | | 0.051 | 1.3 |
| P-185 | | | 0.017 | 1.5 |
| P-186 | 0.034 | 13.9 | 0.029 | 5.7 |
| P-187 | | | 0.020 | 1.4 |
| P-188 | | | 0.006 | 1.1 |
| P-189 | | | 0.054 | 25.3 |
| P-190 | | | 0.158 | 24.9 |
| P-191 | | | 1.792 | 20.4 |
| P-192 | | | 0.058 | 1.0 |
| P-193 | | | 0.048 | <0.5 |
| P-194 | | | 4.043 | 39.8 |
| P-195 | | | 0.006 | 2.2 |
| P-196 | | | 0.167 | 3.5 |
| P-197 | | | 0.032 | 2.7 |
| P-198 | | | 0.010 | 1.2 |
| P-199 | | | 0.002 | 2.7 |
| P-200 | | | 0.013 | 1.5 |
| P-201 | | | 0.002 | 0.6 |
| P-202 | | | 0.010 | 1.4 |
| P-203 | | | 0.011 | 3.0 |
| P-204 | | | 0.007 | 1.8 |
| P-205 | | | 0.030 | 1.1 |
| P-206 | | | 0.002 | 1.1 |
| P-207 | | | >5.000 | 1.3 |
| P-208 | | | 0.009 | 1.4 |
| P-209 | | | 0.001 | 0.9 |
| P-210 | | | 0.015 | >95.0 |
| P-211 | | | 0.003 | 1.1 |
| P-212 | | | 0.003 | 1.5 |
| P-213 | | | 0.002 | 1.7 |
| P-214 | | | <0.001 | <0.5 |
| P-215 | | | <0.001 | 0.7 |
| P-216 | | | 0.001 | 1. |
| P-217 | | | 0.009 | 1.6 |
| P-218 | | | 0.006 | 4.3 |
| P-219 | | | 0.006 | 1.8 |
| P-220 | | | 0.006 | 4.3 |
| P-221 | | | 0.005 | 1.4 |
| P-222 | | | 0.007 | 2.1 |
| P-223 | | | <0.001 | 2.1 |
| P-224 | | | 0.06 | 15.9 |
| P-225 | | | 0.133 | 3.5 |
| P-226 | | | 0.005 | 2.1 |
| P-227 | | | 0.002 | 3.0 |
| P-228 | | | 0.009 | 3.6 |
| P-229 | | | 0.002 | 3.6 |
| P-230 | | | 0.007 | 2.8 |
| P-231 | | | 0.001 | 1.37 |
| P-232 | | | 0.065 | 7.1 |
| P-233 | | | 0.008 | 2.8 |
| P-234 | | | 0.013 | 3.1 |
| P-235 | | | 0.029 | 2.6 |
| P-236 | | | 0.269 | 23.3 |
| P-237 | | | 0.007 | 2.8 |
| P-238 | | | 0.025 | 1.1 |
| P-239 | | | 0.165 | 8.6 |
| P-240 | | | 0.297 | 14.5 |
| P-241 | | | 0.039 | 3.5 |
| P-242 | | | 0.023 | 2.5 |
| P-243 | | | 0.159 | 3.9 |
| P-244 | | | 0.056 | 3.5 |
| P-245 | | | 0.012 | 2.6 |
| P-246 | | | 0.0068 | 4.1 |
| P-247 | | | 0.1909 | 13.4 |
| P-248 | | | 0.0040 | 4.1 |
| P-249 | | | 0.0023 | 1.9 |
| P-250 | | | 0.0012 | 2.4 |
| P-251 | | | 0.0098 | 1.9 |
| P-252 | | | 0.0054 | 1.7 |
| P-253 | | | 0.0487 | 9.4 |
| P-254 | | | 0.0217 | 91.0 |
| P-255 | | | 0.0124 | 90.4 |
| P-256 | | | 0.0026 | 1.9 |
| P-257 | | | 0.0928 | 34.7 |
| P-258 | | | 0.0069 | 2.0 |
| P-259 | | | 0.0261 | 89.5 |
| P-260 | | | 0.0170 | 7.6 |
| P-261 | | | | |
| P-262 | | | 0.0171 | 8.7 |
| P-263 | | | 0.0257 | 9.0 |
| P-264 | | | 0.0072 | 94.6 |
| P-265 | | | 1.0000 | 93.2 |
| P-266 | | | 0.0062 | 2.2 |
| P-267 | | | 0.0947 | 54.7 |
| P-268 | | | 0.0021 | −0.2 |
| P-269 | | | 0.0118 | 6.3 |
| P-270 | | | 0.0065 | 0.2 |
| P-271 | | | 0.0188 | 9.9 |
| P-272 | | | 0.0103 | 10.2 |
| P-273 | | | 0.0049 | 5.6 |
| P-274 | | | 0.0058 | 3.5 |
| P-275 | | | 0.0146 | 2.9 |
| P-276 | | | 0.0114 | 6.0 |
| P-277 | | | | |
| P-278 | | | 0.0084 | 3.9 |
| P-279 | | | 0.9320 | 71.8 |
| P-280 | | | 0.1073 | 47.7 |
| P-281 | | | 0.1056 | 24.0 |
| P-282 | | | 0.0034 | 1.0 |
| P-283 | | | 0.0057 | 5.2 |
| P-284 | | | 0.0038 | 0.6 |
| P-285 | | | 0.0185 | 10.4 |
| P-286 | | | 0.0085 | 6.6 |
| P-287 | | | 1.3530 | 51.0 |
| P-288 | | | 0.0071 | 4.7 |
| P-289 | | | 0.0113 | 7.7 |
| P-290 | | | 0.2525 | 33.6 |
| P-291 | | | 0.0577 | −4.4 |
| P-292 | | | 0.0320 | 7.2 |
| P-293 | | | 0.0658 | 10.5 |
| P-294 | | | 1.2287 | 76.0 |
| P-295 | | | 0.3499 | 39.8 |
| P-296 | | | | |
| P-297 | | | 0.0493 | 6.5 |
| P-298 | | | 0.0827 | 3.2 |
| P-299 | | | 0.0364 | 3.5 |
| P-300 | | | 0.2431 | 11.7 |
| P-301 | | | 0.1026 | 17.8 |
| P-302 | | | 0.0105 | 3.3 |
| P-303 | | | 0.1111 | 90.1 |
| P-304 | | | 0.0119 | 5.0 |
| P-305 | | | 0.0002 | 84.3 |
| P-306 | | | 0.0037 | 1.5 |
| P-307 | | | 0.0066 | 86.3 |
| P-308 | | | 0.0021 | 3.5 |
| P-309 | | | 1.0000 | 96.8 |
| P-310 | | | 0.0036 | 1.3 |
| P-311 | | | 0.0030 | −1.0 |
| P-312 | | | 0.0132 | 7.9 |
| P-313 | | | 0.2443 | 44.9 |
| P-314 | | | 0.0218 | 6.5 |
| P-315 | | | 0.0022 | 1.4 |
| P-316 | | | | |
| P-317 | | | 0.0013 | 2.0 |
| P-318 | | | 0.3742 | 47.8 |
| P-319 | | | 1.0000 | 87.9 |
| P-320 | | | 0.0073 | 1.6 |
| P-321 | | | 0.6453 | 24.2 |
| P-322 | | | 0.0165 | 4.4 |
| P-323 | | | 0.0967 | 12.0 |
| P-324 | | | 1.0000 | 107.9 |
| P-325 | | | 0.0038 | 1.6 |
| P-326 | | | 0.0040 | 0.3 |
| P-327 | | | 0.0028 | 2.2 |
| P-328 | | | 0.0025 | 2.5 |
| P-329 | | | 0.0059 | 2.6 |
| P-330 | | | 0.0174 | 2.3 |
| P-331 | | | 0.0018 | −2.2 |
| P-332 | | | 0.2608 | 20.7 |
| P-333 | | | 0.3335 | 26.8 |
| P-334 | | | 0.0587 | 23.5 |
| P-335 | | | 0.0298 | 5.1 |
| P-336 | | | 0.0251 | 5.5 |
| P-337 | | | 0.0038 | 2.7 |
| P-338 | | | 0.5013 | 61.2 |
| P-339 | | | 0.0186 | 93.1 |
| P-340 | | | 0.5825 | 22.4 |
| P-341 | | | 0.0024 | 81.8 |
| P-342 | | | 0.0221 | 7.1 |
| P-343 | | | 0.1033 | 87.0 |
| P-344 | | | 0.0912 | 8.9 |
| P-345 | | | 1.0000 | 87.0 |
| P-346 | | | 0.0928 | 17.5 |
| P-347 | | | | |
| P-348 | | | 0.0949 | 13.1 |
| P-349 | | | 0.0297 | 14.6 |
| P-350 | | | 0.0022 | 3.7 |
| P-351 | | | 1832 | 69.0 |
| P-352 | | | 0.0038 | 2.1 |
| P-353 | | | 1.0000 | 88.5 |
| P-354 | | | 0.0137 | 4.0 |
| P-355 | | | 0.3269 | 58.0 |
| P-356 | | | 0.0406 | 8.6 |
| P-357 | | | 0.1786 | 26.7 |
| P-358 | | | 0.0048 | 0.6 |
| P-359 | | | 0.0025 | −1.9 |
| P-360 | | | 0.0276 | 4.5 |
| P-361 | | | 0.005 | 1.2 |
| P-362 | | | 0.2383 | 14.9 |
| P-363 | | | 0.0076 | 5.0 |
| P-364 | | | 0.0018 | 3.1 |
| P-365 | | | 0.0023 | 2.0 |
| P-366 | | | 0.01 | 2.0 |
| P-367 | | | 0.1163 | 7.9 |
| P-368 | | | 0.0044 | 2.9 |
| P-369 | | | 0.0022 | 3.0 |
| P-370 | | | 0.0305 | 1.2 |
| P-371 | | | 0.0087 | 4.1 |
| P-372 | | | 0.2354 | 40.1 |
| P-373 | | | 1.0000 | 72.5 |
| P-375 | | | 0.0065 | 2.5 |
| P-376 | | | 0.0303 | 7.2 |
| P-377 | | | 0.0083 | 3.4 |
| P-378 | | | 0.013 | 2.2 |
| P-379 | | | 0.0040 | 1.8 |
| P-380 | | | 0.0894 | 12.1 |
| P-381 | | | 0.0083 | 2.1 |
| P-382 | | | 0.0280 | 4.1 |
| P-383 | | | 0.0313 | 5.6 |
| P-384 | | | 0.0454 | 6.5 |
| P-385 | | | 0.0087 | −1.9 |
| P-386 | | | 0.0242 | 6.7 |
| P-387 | | | 0.0141 | 9.8 |
| P-388 | | | 0.0111 | 3.5 |
| P-389 | | | 0.1798 | 18.9 |
| P-390 | | | 0.0028 | −1.8 |
| P-391 | | | 0.0037 | −0.8 |
| P-392 | | | 0.0087 | 3.5 |
| P-393 | | | 0.0061 | −3.4 |
| P-394 | | | 0.006 | 1.4 |
| P-395 | | | 0.0093 | 2.6 |
| P-396 | | | 0.1000 | 8.9 |
| P-397 | | | 0.0143 | 6.2 |
| P-398 | | | 0.0240 | 5.8 |
| P-399 | | | 0.0431 | 8.2 |
| P-400 | | | 0.0279 | 7.8 |
| P-401 | | | 0.0120 | 0.5 |
| P-402 | | | 0.0033 | 1.9 |
| P-403 | | | 0.0025 | 3.7 |
| P-405 | | | 0.0048 | 3.1 |
| P-406 | | | 0.0076 | 1.7 |
| P-407 | | | 0.0358 | 5.6 |
| P-408 | | | 0.0426 | 7.8 |
| P-409 | | | 0.0825 | 8.3 |
| P-410 | | | | |
| P-411 | | | 1.0000 | 90.4 |
| P-412 | | | 0.1602 | 15.2 |
| P-413 | | | 0.0051 | −1.3 |
| P-414 | | | 2.3474 | 71.4 |
| P-415 | | | 0.1915 | 37.5 |
| P-416 | | | 0.3551 | 34.7 |
| P-417 | | | 0.0514 | 1.8 |
| P-418 | | | 0.0149 | 1.8 |
| P-419 | | | 0.0316 | −0.8 |
| P-420 | | | 0.0262 | 1.7 |
| P-421 | | | 0.0006 | −3.1 |
| P-422 | | | 0.9790 | 39.6 |
| P-423 | | | 0.1430 | 8.6 |
| P-424 | | | 0.0019 | 5.3 |
| P-425 | | | 0.1240 | 0.8 |
| P-426 | | | 1.0000 | 41.0 |
| P-427 | | | 0.0404 | 1.8 |
| P-428 | | | 0.0032 | −0.6 |
| P-429 | | | 0.0594 | 5.4 |
| P-430 | | | 0.0055 | −0.4 |
| P-432 | | | 0.0013 | 1.8 |
| P-433 | | | 0.7005 | 37.6 |
| P-434 | | | 0.0299 | 1.9 |
| P-435 | | | 0.1227 | 52.3 |
| P-436 | | | 0.0003 | −5.4 |
| P-437 | | | 0.0004 | 0.0 |
| P-438 | | | 0.0034 | 2.2 |
| P-439 | | | 0.0924 | 11.2 |
| P-440 | | | 0.0537 | 2.9 |
| P-441 | | | 0.0026 | −0.1 |
| P-442 | | | 0.0044 | −2.0 |
| P-443 | | | 0.0045 | −0.6 |
| P-444 | | | 0.0052 | −1.6 |
| P-445 | | | 0.0658 | 4.0 |
| P-446 | | | 0.0014 | −1.4 |
| P-447 | | | 0.0006 | 0.8 |
| P-448 | | | 0.0321 | 3.9 |
| P-449 | | | 0.4239 | 23.7 |
| P-452 | | | 0.0019 | 0.5 |
| P-453 | | | 0.0046 | 0.6 |
| P-454 | | | 0.0136 | 1.1 |
| P-455 | | | 0.0078 | −5.9 |
| P-458 | | | 0.1284 | 15.0 |
| P-459 | | | 0.0025 | −0.5 |
| P-461 | | | 0.0037 | 5.4 |
| P-462 | | | 0.0061 | 3.3 |
| P-463 | | | 0.0001 | −3.4 |
| P-464 | | | 0.0007 | −7.1 |
| P-465 | | | 0.2934 | 18.9 |
| P-468 | | | 0.0172 | 2.3 |
| P-470 | | | 0.0297 | 10.6 |
| P-471 | | | 0.0879 | 7.7 |
| P-473 | | | 1.000 | 15.5 |
| P-474 | | | 0.070 | 5.0 |
| P-475 | | | 0.047 | 6.4 |
| P-476 | | | 0.002 | 0.9 |
| P-477 | | | 0.263 | 18.6 |
| P-478 | | | 0.161 | 17.1 |
| P-480 | | | 0.026 | 0.8 |
| P-481 | | | 0.038 | 9.4 |
| P-483 | | | 0.011 | 3.4 |
| P-484 | | | 1.000 | 52.3 |
| P-486 | | | 0.200 | 24.9 |
| P-487 | | | 0.008 | 2.7 |
| P-488 | | | 4.445 | 41.7 |
| P-489 | | | 1.000 | 84.3 |
| P-490 | | | 1.000 | 81.6 |
| P-491 | | | 0.156 | 14.9 |
| P-492 | | | 0.035 | −0.2 |
| P-494 | | | 0.110 | 21.2 |
| P-495 | | | 0.110 | 2.7 |
| P-496 | | | 0.127 | 2.7 |
| P-497 | | | 0.239 | 13.6 |
| P-498 | | | 0.002 | −2.8 |
| P-499 | | | 0.172 | 13.6 |
| P-500 | | | 0.0434 | −0.52 |
| P-501 | | | 0.0022 | −2.28 |
| P-502 | | | 0.0012 | −0.79 |
| P-503 | | | 1.0000 | 82.16 |
| P-504 | | | 0.1189 | 28.37 |
| P-505 | | | 0.1972 | 9.83 |
| P-506 | | | 1.0000 | 65.39 |
| P-507 | | | 0.0628 | −1.72 |
| P-508 | | | | |
| P-509 | | | | |
| P-510 | | | | |
| P-511 | | | | |
| P-512 | | | | |
| P-513 | | | 0.008 | 5.2 |

Blank entries indicate “not determined”.

### Example B4. Pharmacokinetic Studies in Mice

Due to the higher molecular weight and lipophilicity, targeted protein degraders generally occupy a physiochemical property space that is beyond Lipinski's rule of 5 and as consequence are expected to have poor oral bioavailability and overall pharmacokinetic properties. Compounds of the present disclosure can overcome these liabilities as demonstrated by the highly favorable PK properties shown for the selected compounds. The plasma pharmacokinetics (PK) of P-394, P-155, P-139, and P-160 were characterized in female C₅₇B16 mice following intravenous (IV; 0.5 mg/kg) administration. Plasma from the tail vein was withdrawn in composite fashion: one group of mice (n=3) provided samples at 2 min, 30 min, 2 h, and 8 h, while another group (n=3) provided samples at 15 min, 1 h, 4 h, and 24 h. The concentrations of test article in these samples were measured using reverse-phase LC-MS-MS and then plotted as a function of time. The resulting concentration-time data was processed using noncompartmental analysis for the calculation of clearance (Cl), volume of distribution at steady state (Vss), half-life, mean residence time (MRT) and area under the time curve (AUC).

Similarly, the PK was characterized following oral (PO; 3 mg/kg) administration. Again, plasma from the tail vein was withdrawn in composite fashion: one group of mice (n=3) provided samples at 15 min, 1 h, 4 h, and 8 h, while another group (n=3) provided samples at min, 2 h, and 24 h. Oral bioavailability (F_{PO}) was calculated by comparing the results of the studies described, dividing the dose-normalized AUC_{LAST,PO} by the dose-normalized AUC_{LAST,IV}. The results of the PK study in mice for compounds P-394, P-155, P-139, and P-160 are shown in Tables 75-78 below and highlight the highly favorable PK properties and oral bioavailability for molecules of this size.

TABLE 75
Mouse pharmacokinetics for P-394; IV vehicle (85%
D5W, 5% DMSO, 10% SOLUTOL (solution); PO vehicle
(0.5% MC & 0.25% TWEEN 80 in water (solution))

| | IV (0.50 mg/kg) | PO (3.00 mg/kg) |
|---|---|---|
| Clp (mL/min/kg) | 11.8 | |
| V_{SS} (L/kg) | 1.58 | |
| Half-life (h) | 1.7 | |
| MRT (h) | 2.2 | |
| AUC_{LAST} (nM · h) | 884 | 1410 |
| C_{MAX} (nM) | | 559 |
| T_{MAX} (h) | | 0.25 |
| F_{PO} (%) | | 26.6 |

TABLE 76
Mouse pharmacokinetics for P-155; IV vehicle (85% D5W, 5%
DMSO & 10% SOLUTOL (clear solution)); PO vehicle (0.5%
MC & 0.25% TWEEN 80 in water (homogeneous suspension))

| | IV (0.50 mg/kg) | PO (3.0 mg/kg) |
|---|---|---|
| Clp (mL/min/kg) | 5.45 | |
| V_{SS} (L/kg) | 1.08 | |
| Half-life (h) | 4.1 | |
| MRT (h) | 3.3 | |
| AUC_{LAST} (nM · h) | 2010 | 13700 |
| C_{MAX} (nM) | | 3010 |
| T_{MAX} (h) | | 2 |
| F_{PO} (%) | | ~100 |

TABLE 77
Mouse Pharmacokinetics P-139; IV vehicle (85% D5W,
5% DMSO, 10% SOLUTOL (solution); PO vehicle (0.5%
MC & 0.25% TWEEN 80 in water (solution)

| | IV (0.50 mg/kg) | PO (3.0 mg/kg) |
|---|---|---|
| Clp (mL/min/kg) | 10.6 | |
| V_{SS} (L/kg) | 2.91 | |
| Half-life (h) | 2.98 | |
| MRT (h) | 4.59 | |
| AUC_{LAST} (nM · h) | 1061.56 | 2465.81 |
| C_{MAX} (nM) | | 380 |
| T_{MAX} (h) | | 2 |
| F_{PO} (%) | | 39 |

TABLE 78
Mouse pharmacokinetics P-160; IV vehicle (85% D5W, 5%
DMSO & 10% SOLUTOL (clear solution)); PO vehicle (0.5%
MC & 0.25% TWEEN 80 in water (homogeneous suspension)

| | IV (0.50 mg/kg) | PO (3.0 mg/kg) |
|---|---|---|
| Clp (mL/min/kg) | 14.9 | |
| V_{SS} (L/kg) | 3.37 | |
| Half-life (h) | 3.2 | |
| MRT (h) | 3.8 | |
| AUC_{LAST} (nM · h) | 739 | 1400 |
| C_{MAX} (nM) | | 245 |
| T_{MAX} (h) | | 2 |
| F_{PO} (%) | | 31.6 |

Although the present invention has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

1. A compound of Formula (I′): 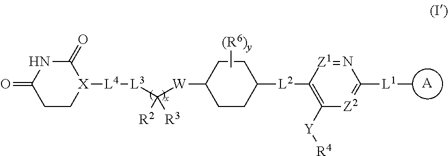
or a pharmaceutically acceptable salt thereof, wherein:
Ring A is phenyl, monocyclic 5- to 6-membered heteroaryl, or fused bicyclic 9- to 10-membered heteroaryl or heterocyclyl, wherein the heteroaryl and heterocyclyl contain 1-4 heteroatoms independently selected from N, O, and S, each of which is optionally substituted by 1-3 R⁰ groups;
each R⁰ is independently selected from halo, —CN, —NH₂, —NH(C₁-C₆ alkyl), C₁-C₆ alkyl, C₃-C₆ cycloalkyl, -(6- to 10-membered bridged heterocyclylene)-, and C₁-C₆ alkoxy, wherein the heterocyclylene contains 1-3 heteroatoms selected from N and O, or two R⁰ groups are taken together to form an oxo group;
L¹ is —NH— or a bond;
L² is —NHC(O)—, —C(O)NH—, —SO₂NH—, —NHSO₂—, or —(C₁-C₆ alkylene)_{z}(5-membered heteroarylene)-, wherein the heteroarylene contains 1-3 heteroatoms selected from N, O, and S;
L³ is —NR⁹ (C₁-C₆ alkylene)NR⁹—, —NR⁹C(O)(C₁-C₆ alkylene)_{z}(4- to 7-membered heterocyclylene)-, -(4- to 7-membered heterocyclylene)CR¹¹R¹²—, -(4- to 7-membered heterocyclylene)(CO)_{z}—, -(4- to 7-membered heterocyclylene)(NR⁹)_{z}—, —(NR⁹)_{z}(4- to 7-membered heterocyclylene)(C₁-C₆ alkylene)_{z}-, —NR⁹ (C₁-C₆ alkylene)_{z}(4- to 7-membered heterocyclylene)-, —NR⁹C(O)(phenylene)NR⁹—, —(C₁-C₆ alkylene)_{z}(4- to 7-membered heterocyclylene)(C₁-C₆ alkylene)_{z}-, —O(C₁-C₆ alkylene)_{z}(4- to 7-membered heterocyclylene)(C₁-C₆ alkylene)_{z}-, -(6- to 10-membered bridged heterocyclylene)(C₁-C₆ alkylene)_{z}-, -(7- to 10-membered fused bicyclic heterocyclylene)(C₁-C₆ alkylene)_{z}-, or —(O)_{z}(6- to 10-membered spiro heterocyclylene)(C₁-C₆ alkylene)_{z}-, wherein the heterocyclylene contains 1-3 heteroatoms selected from N and O and is optionally substituted by 1-5 R⁷ groups;
L⁴ is 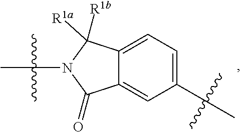
phenylene, —N(H)(phenylene), 5- to 6-membered heteroarylene, —N(H)(5- to 6-membered heteroarylene)-, 8- to 10-membered fused bicyclic heteroarylene, or 5- to 6-membered heterocyclylene, wherein the phenylene, heteroarylene, or heterocyclylene is optionally substituted by 1-4 R⁷ groups, and wherein the heteroarylene and heterocyclylene contain 1-3 heteroatoms selected from N, S, and O;
R^{1a} and R^{1b} are each H or are taken together to form an oxo group;
R² and R³ are independently H, C₁-C₆ alkyl, or halo, or R² and R³ are taken together to form an oxo group;
or R³ and R¹¹ are taken together to form a C₃-C₆ cycloalkylene group;
Y is NH, O, or a bond;
R⁴ is C₃-C₆ cycloalkyl, C₁-C₆ alkylene-(C₃-C₆ cycloalkyl), 4- to 6-membered heterocyclyl, C₁-C₆ alkylene-(4- to 6-membered heterocyclyl), 5- to 6-membered heteroaryl, C₁-C₆ alkylene-(5- to 6-membered heteroaryl), C₁-C₆ alkyl, C₁-C₆ haloalkyl, C₁-C₆ alkyl-OH, or C₁-C₆ alkyl-CN, wherein the heterocyclyl and heteroaryl contain 1-3 heteroatoms selected from N and O, and wherein the cycloalkyl, heterocyclyl, or heteroaryl is optionally substituted by 1-5 R⁸ groups;
W is O, —NR⁵—, or a bond;
R⁵ is H or C₁-C₆ alkyl;
each R⁶ is independently C₁-C₆ alkyl, halo, or —OH, or two R⁶ groups are taken together to form a bridging C1-C₃ alkylene group;
each R⁷ is independently C₁-C₆ alkyl, halo, C₁-C₆ haloalkyl, or —OH, or two R⁷ groups are taken together to form an oxo group;
each R⁸ is independently —SO₂(C₁-C₆ alkyl), —C(O)(C₁-C₆ alkyl), C₁-C₆ alkyl, C₁-C₆ haloalkyl, halo, —CN, or —OH;
each R⁹ is independently H or C₁-C₆ alkyl;
each R¹⁰ is independently C₁-C₆ alkoxy, C₁-C₆ alkyl, halo, or —OH, or two R¹⁰ groups are taken together to form an oxo group;
each R¹¹ and R¹² is independently H, halo, C₃-C₆ cycloalkyl, —OH, —NH(C₁-C₆ alkyl), C₁-C₆ haloalkyl, or C₁-C₆ alkyl;
or R¹¹ and R³ are taken together to form a C₃-C₆ cycloalkylene group;
x is 0 or 1;
y is 0, 1, 2, 3, 4, or 5;
each z is independently 0 or 1;
X is N or CR¹³;
R¹³ is H, halo, —OH, or C₁-C₆ alkyl;
Z¹ is CH or N; and
Z² is CH or N,
provided that Z¹ and Z² are not both N.

2. The compound of claim 1, or a pharmaceutically acceptable salt thereof, wherein the compound is of Formula (I): 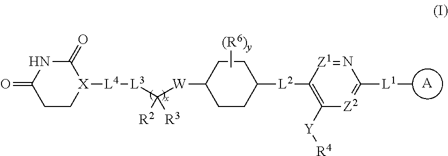
wherein:
Ring A is phenyl, monocyclic 5- to 6-membered heteroaryl, or fused bicyclic 9- to 10-membered heteroaryl or heterocyclyl, wherein the heteroaryl and heterocyclyl contain 1-4 heteroatoms independently selected from N, O, and S, each of which is optionally substituted by 1-3 R⁰ groups;
each R⁰ is independently selected from halo, —CN, —NH₂, —NH(C₁-C₆ alkyl), C₁-C₆ alkyl, C₃-C₆ cycloalkyl, and C₁-C₆ alkoxy, or two R⁰ groups are taken together to form an oxo group;
L¹ is —NH— or a bond;
L² is —NHC(O)—, —C(O)NH—, —SO₂NH—, —NHSO₂—, or 5-membered heteroarylene containing 1-3 heteroatoms selected from N, O, and S;
L³ is —NR⁹ (C₁-C₆ alkylene)NR⁹—, —NR⁹C(O)(C₁-C₆ alkylene)_{z}(4- to 7-membered heterocyclylene)-, -(4- to 7-membered heterocyclylene)CR¹¹R¹²—, -(4- to 7-membered heterocyclylene)(CO)_{z}—, -(4- to 7-membered heterocyclylene)(NR⁹)_{z}—, —(NR⁹)_{z}(4- to 7-membered heterocyclylene)(C₁-C₆ alkylene)_{z}-, —NR⁹ (C₁-C₆ alkylene)_{z}(4- to 7-membered heterocyclylene)-, —NR⁹C(O)(phenylene)NR⁹—, —(C₁-C₆ alkylene)_{z}(4- to 7-membered heterocyclylene)-, —O(C₁-C₆ alkylene)_{z}(4- to 7-membered heterocyclylene)-, -(6- to 10-membered bridged heterocyclylene)(C₁-C₆ alkylene)_{z}-, -(9- to 10-membered fused bicyclic heterocyclylene)(C₁-C₆ alkylene)_{z}-, or —(O)_{z}(6- to 10-membered spiro heterocyclylene)(C₁-C₆ alkylene)_{z}-, wherein the heterocyclylene contains 1-3 heteroatoms selected from N and O and is optionally substituted by 1-5 R⁷ groups;
L⁴ is 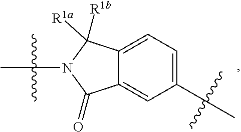
phenylene, 5- to 6-membered heteroarylene, or 5- to 6-membered heterocyclylene, wherein the phenylene, heteroarylene, or heterocyclylene is optionally substituted by 1-4 R¹⁰ groups, and wherein the heteroarylene and heterocyclylene contain 1-3 heteroatoms selected from N and O;
R^{1a} and R^{1b} are each H or are taken together to form an oxo group;
R² and R³ are independently H, C₁-C₆ alkyl, or halo, or R² and R³ are taken together to form an oxo group;
or R³ and R⁴ are taken together to form a C₃-C₆ cycloalkylene group;
Y is NH or O;
R⁴ is C₃-C₆ cycloalkyl, C₁-C₆ alkylene-(C₃-C₆ cycloalkyl), 4- to 6-membered heterocyclyl, C₁-C₆ alkylene-(4- to 6-membered heterocyclyl), 5- to 6-membered heteroaryl,
C₁-C₆ alkylene-(5- to 6-membered heteroaryl), C₁-C₆ alkyl, C₁-C₆ haloalkyl, C₁-C₆ alkyl-OH, or C₁-C₆ alkyl-CN, wherein the heterocyclyl and heteroaryl contain 1-3 heteroatoms selected from N and O, and wherein the cycloalkyl, heterocyclyl, or heteroaryl is optionally substituted by 1-5 R⁸ groups;
W is O, —NR⁵—, or a bond;
R⁵ is H or C₁-C₆ alkyl;
each R⁶ is independently C₁-C₆ alkyl, halo, or —OH, or two R⁶ groups are taken together to form a bridging C1-C₃ alkylene group;
each R⁷ is independently C₁-C₆ alkyl, halo, C₁-C₆ haloalkyl, or —OH, or two R⁷ groups are taken together to form an oxo group;
each R⁸ is independently —SO₂(C₁-C₆ alkyl), —C(O)(C₁-C₆ alkyl), C₁-C₆ alkyl, C₁-C₆ haloalkyl, halo, —CN, or —OH;
each R⁹ is independently H or C₁-C₆ alkyl;
each R¹⁰ is independently C₁-C₆ alkoxy, C₁-C₆ alkyl, halo, or —OH, or two R¹⁰ groups are taken together to form an oxo group;
each R¹¹ and R¹² is independently H, halo, C₃-C₆ cycloalkyl, —OH, —NH(C₁-C₆ alkyl), C₁-C₆ haloalkyl, or C₁-C₆ alkyl;
or R¹¹ and R³ are taken together to form a C₃-C₆ cycloalkylene group;
x is 0 or 1;
y is 0, 1, 2, 3, 4, or 5;
each z is independently 0 or 1;
X is N or CR¹³;
R¹³ is H, halo, or C₁-C₆ alkyl;
Z¹ is CH or N; and
Z² is CH or N,
provided that Z¹ and Z² are not both N.

3. The compound of claim 1, or a pharmaceutically acceptable salt thereof, wherein Ring A is:
(i) phenyl optionally substituted by 1-3 R⁰ groups; and
each R⁰ is independently selected from halo, —CN, —NH₂, —NH(C₁-C₃ alkyl), C₁-C₃ alkyl, C₃-C₆ cycloalkyl, and C₁-C₃ alkoxy;
(ii) a monocyclic 6-membered heteroaryl containing 1-2 heteroatoms independently selected from N and O, and optionally substituted by 1-3 R⁰ groups; and
each R⁰ is independently selected from halo, —CN, —NH₂, —NH(C₁-C₃ alkyl), C₁-C₃ alkyl, C₃-C₆ cycloalkyl, and C₁-C₃ alkoxy; or
(iii) a fused bicyclic 9-membered heteroaryl or heterocyclyl containing 2-4 heteroatoms independently selected from N, O, and S, and optionally substituted by 1-3 R⁰ groups; and
each R⁰ is independently selected from halo, —CN, —NH₂, —NH(C₁-C₃ alkyl), C₁-C₃ alkyl, C₃-C₆ cycloalkyl, -(6- to 8-membered bridged heterocyclylene)-, and C₁-C₃ alkoxy, wherein the heterocyclylene contains 1-3 heteroatoms selected from N and O, or two R⁰ groups are taken together to form an oxo group.

4. The compound of claim 3, or a pharmaceutically acceptable salt thereof, wherein Ring A is:

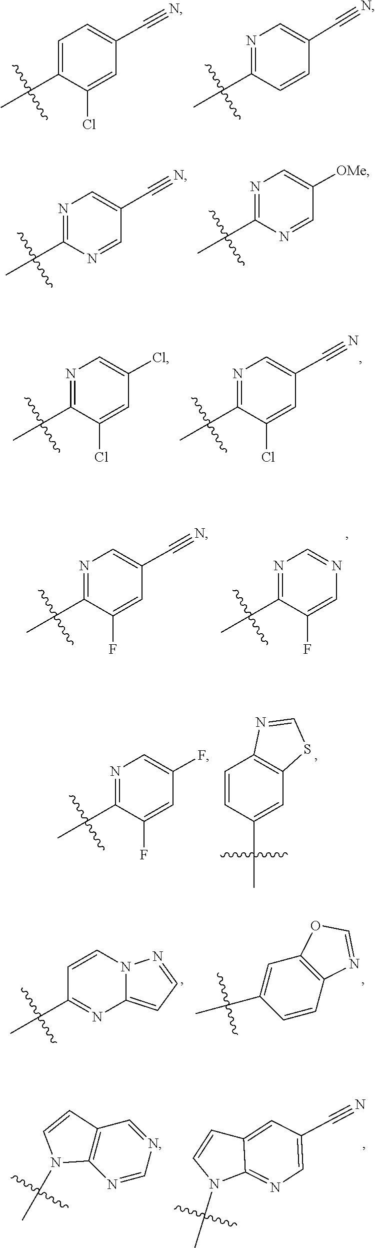

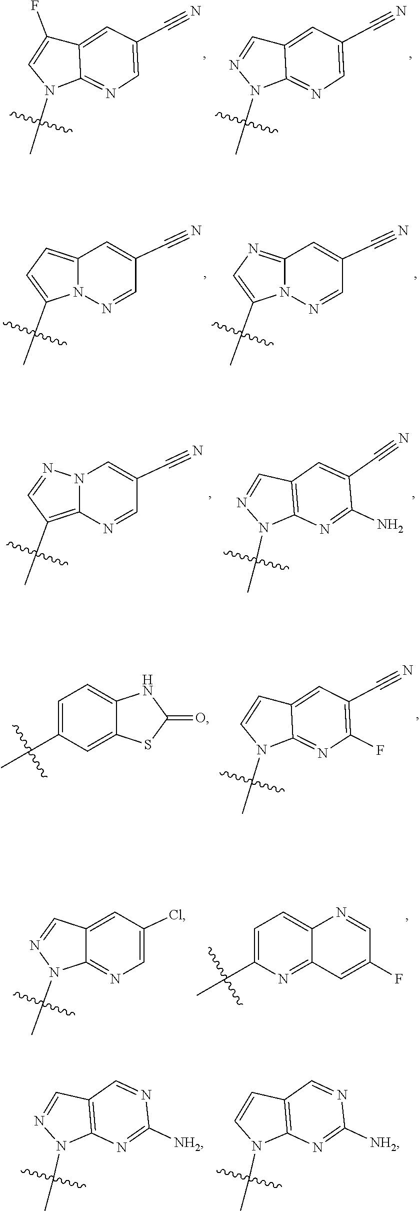

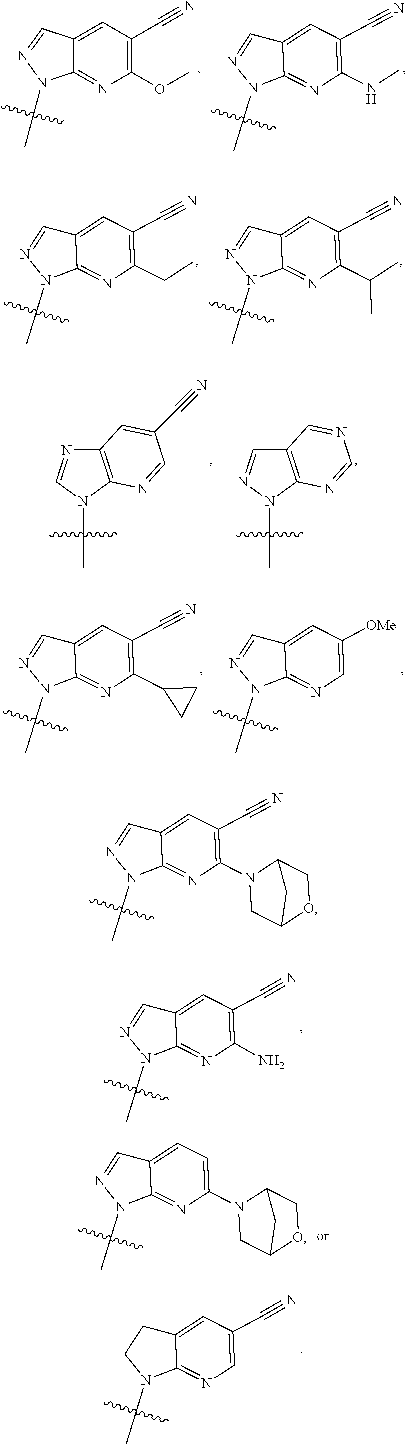

5. The compound of claim 1, or a pharmaceutically acceptable salt thereof, wherein:
L² is —NHC(O)— or —(C₁-C₃ alkylene)_{z}(5-membered heteroarylene)-, wherein the heteroarylene contains 1-3 heteroatoms selected from N and O.

6. The compound of claim 5, or a pharmaceutically acceptable salt thereof, wherein: L² is —NHC(O)—,

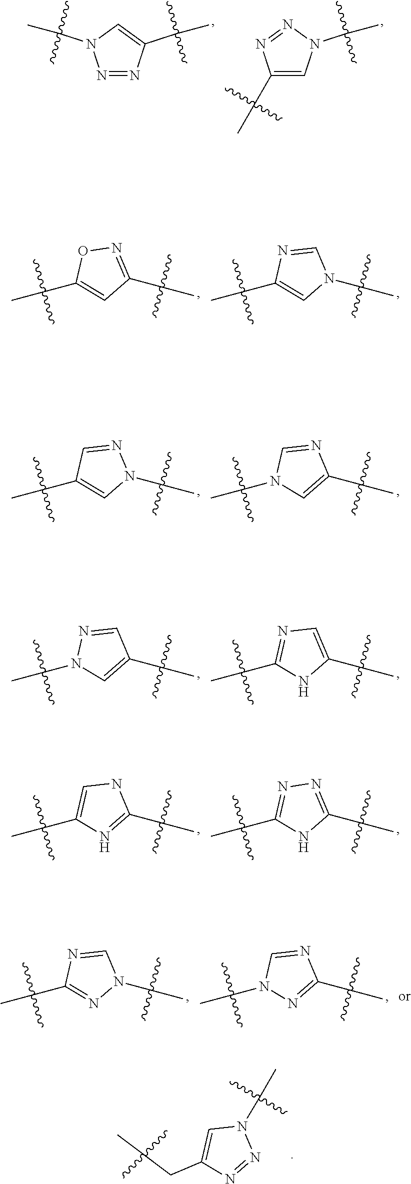

7. The compound of claim 1, or a pharmaceutically acceptable salt thereof, wherein:
L⁴ is

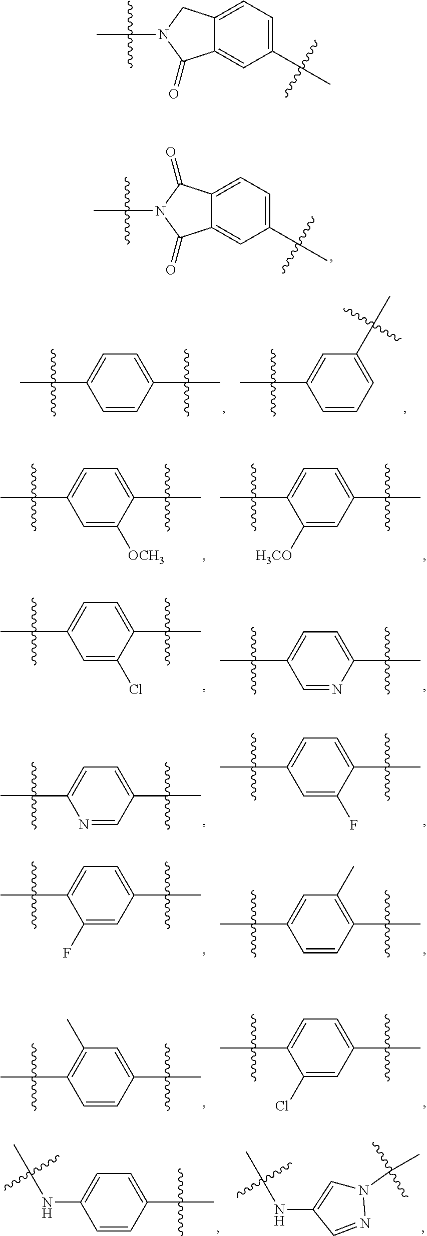

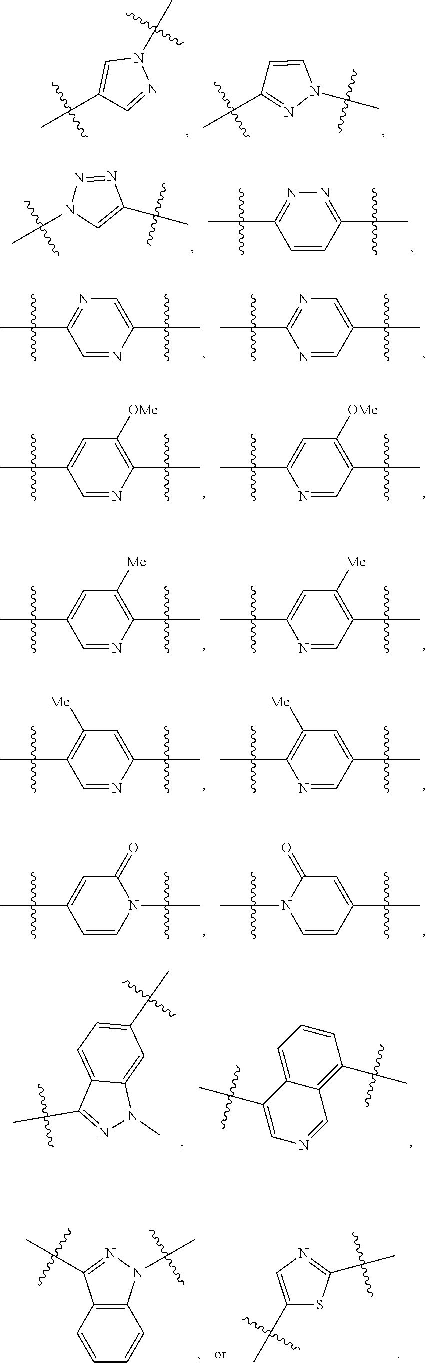

8. The compound of claim 1, or a pharmaceutically acceptable salt thereof, wherein: 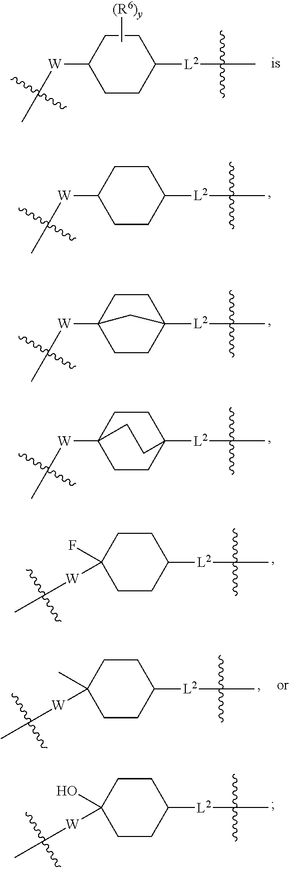
W is O, —NR⁵—, or a bond; and
R⁵ is H or C₁-C₃ alkyl.

9. The compound of claim 1, or a pharmaceutically acceptable salt thereof, wherein:
X is N.

10. The compound of claim 1, or a pharmaceutically acceptable salt thereof, wherein:
X is CR¹³; and
R¹³ is H, halo, —OH, or C₁-C₃ alkyl.

11. The compound of claim 1, or a pharmaceutically acceptable salt thereof, wherein:
Y is NH;
R⁴ is C₃-C₆ cycloalkyl, C₁-C₃ alkylene-(C₃-C₆ cycloalkyl), 4- to 6-membered heterocyclyl, C₁-C₃ alkylene-(4- to 6-membered heterocyclyl), 5- to 6-membered heteroaryl,
C₁-C₃ alkylene-(5- to 6-membered heteroaryl), C₁-C₆ alkyl, C₁-C₆ haloalkyl, C₁-C₆ alkyl-OH, or C₁-C₆ alkyl-CN, wherein the heterocyclyl and heteroaryl contain 1 or 2 heteroatoms selected from N and O, and wherein the cycloalkyl, heterocyclyl, or heteroaryl is optionally substituted by 1-2 R⁸ groups; and
each R⁸ is independently —SO₂(C₁-C₃ alkyl), —C(O)(C₁-C₃ alkyl), C₁-C₃ alkyl, C₁-C₃ haloalkyl, halo, —CN, or —OH.

12. The compound of claim 1, or a pharmaceutically acceptable salt thereof, wherein:
R⁴ is methyl, ethyl, n-propyl, isopropyl, tert-butyl, —CH₂CH(CH₃)₂, —CH₂CF₃, —CH₂CH₂F, —CH₂CF₂CH₃, —CH(CH₃)CF₃, —CH₂CH₂CF₃, —CH(CH₃)CH₂OH, —CH₂C(CH₃)₂OH, —CH₂CN, —CH(CH₃)CN, —C(CH₃)₂CN, —CH(CH₂CH₃)CN, —CH₂CH(CH₃)CN,

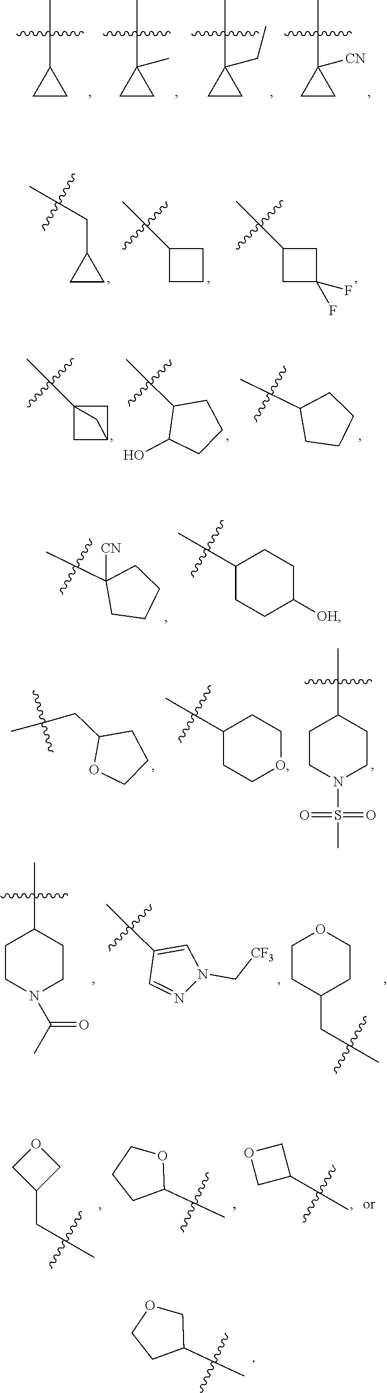

13. The compound of claim 1, or a pharmaceutically acceptable salt thereof, wherein:
L³ is —NR⁹ (C₁-C₃ alkylene)NR⁹—, —NR⁹C(O)(C₁-C₃ alkylene)_{z}(4- to 7-membered heterocyclylene)-, -(4- to 7-membered heterocyclylene)CR¹¹R¹²—, -(4- to 7-membered heterocyclylene)(CO)_{z}—, -(4- to 7-membered heterocyclylene)(NR⁹)_{z}—, —(NR⁹)_{z}(4- to 7-membered heterocyclylene)(C₁-C₃ alkylene)_{z}-, —NR⁹ (C₁-C₃ alkylene)_{z}(4- to 7-membered heterocyclylene)-, —NR⁹C(O)(phenylene)NR⁹—, —(C₁-C₃ alkylene)_{z}(4- to 7-membered heterocyclylene)(C₁-C₃ alkylene)_{z}-, —O(C₁-C₃ alkylene)_{z}(4- to 7-membered heterocyclylene)(C₁-C₃ alkylene)_{z}-, -(6- to 10-membered bridged heterocyclylene)(C₁-C₃ alkylene)_{z}-, -(7- to 10-membered fused bicyclic heterocyclylene)(C₁-C₆ alkylene)_{z}-, or —(O)_{z}(6- to 10-membered spiro heterocyclylene)(C₁-C₃ alkylene)_{z}-, wherein the heterocyclylene contains 1-3 heteroatoms selected from N and O and is optionally substituted by 1 or 2 R⁷ groups;
each z is independently 0 or 1;
each R⁹ is independently H or C₁-C₃ alkyl;
each R⁷ is independently C₁-C₃ alkyl, halo, C₁-C₃ haloalkyl, or —OH, or two R⁷ groups are taken together to form an oxo group; and
each R¹¹ and R¹² is independently H or —CH₃.

14. The compound of claim 13, or a pharmaceutically acceptable salt thereof, wherein:
L³ is

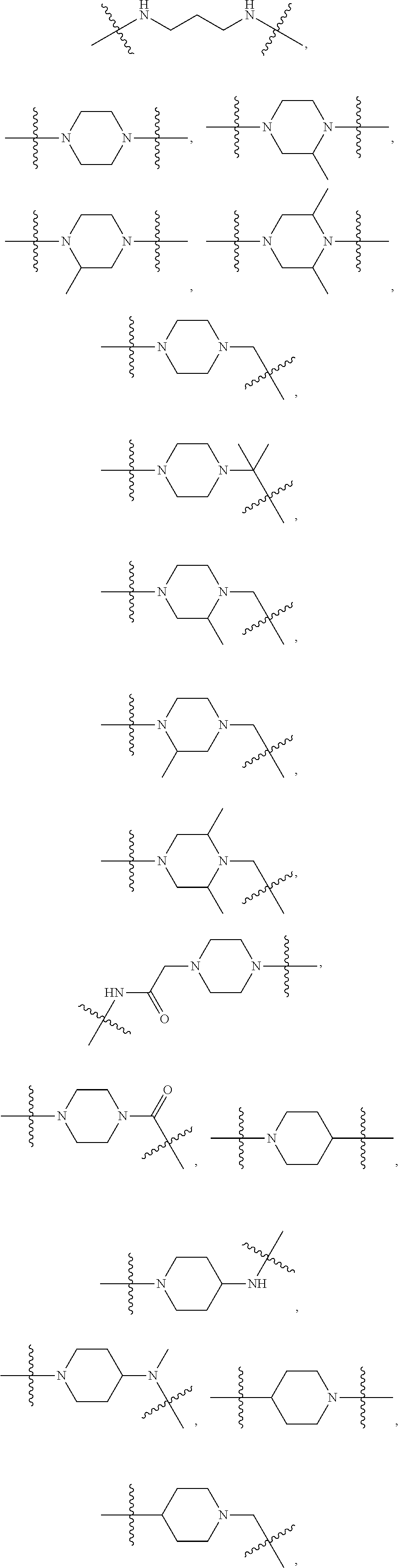

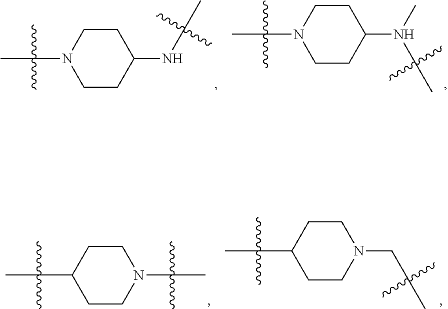

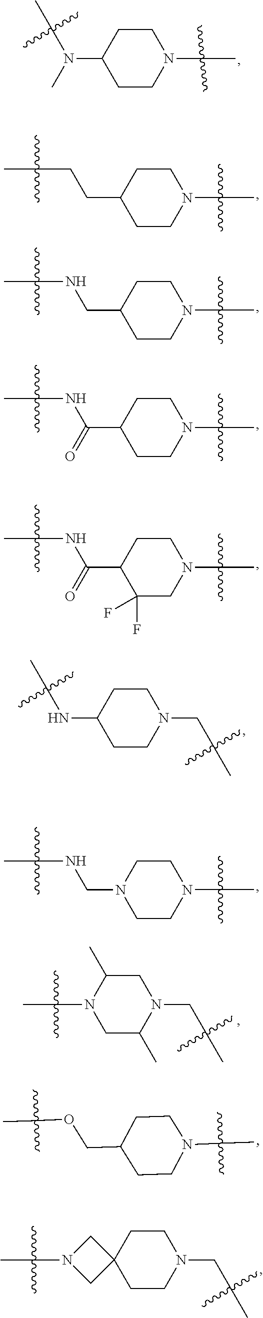

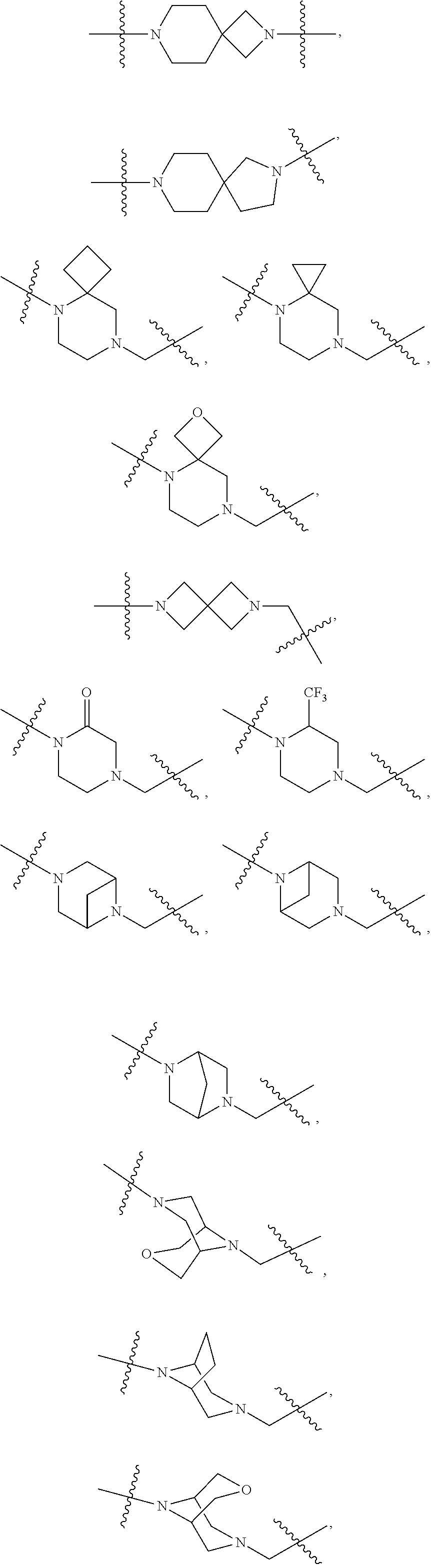

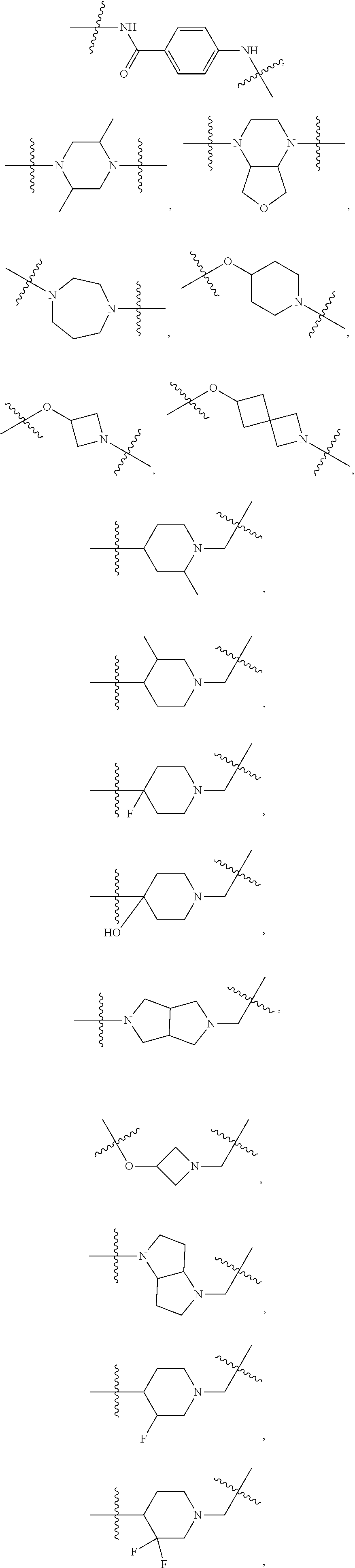

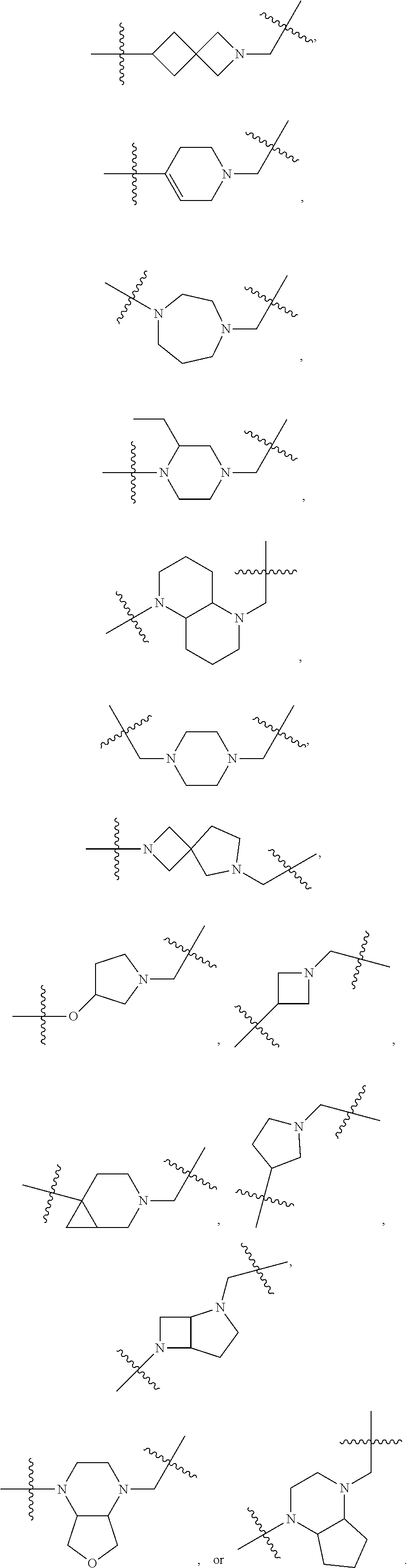

15. The compound of claim 1, or a pharmaceutically acceptable salt thereof, wherein the compound is of Formula (Ia) or (Ia′): 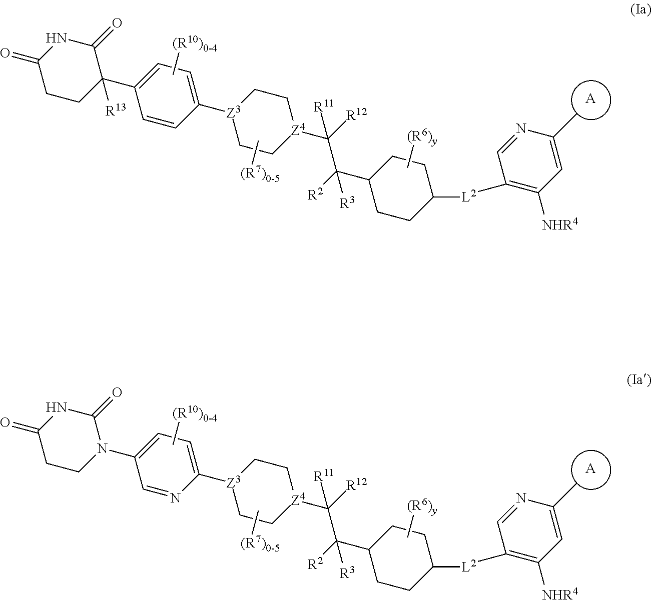
wherein:
Ring A is a fused bicyclic 9- to 10-membered heteroaryl containing 2-4 heteroatoms independently selected from N, O, and S, optionally substituted by 1-3 R⁰ groups;
R⁴ is C₁-C₆ alkyl, C₁-C₆ haloalkyl, C₁-C₆ alkyl-OH, or C₁-C₆ alkyl-CN; and
Z³ and Z⁴ are independently N or CH, provided that at least one of Z³ and Z⁴ is N.

16. The compound of claim 15, or a pharmaceutically acceptable salt thereof, wherein the compound is of Formula (If) or (If):

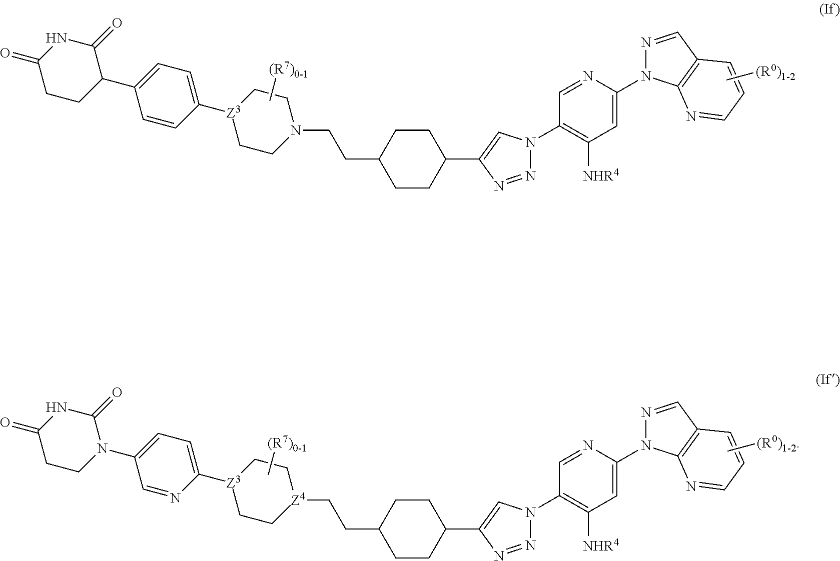

17. A compound selected from the compounds of Table 1 or a pharmaceutically acceptable salt thereof.

18. A pharmaceutical composition comprising the compound of claim 1, or a pharmaceutically acceptable salt thereof, and a pharmaceutically acceptable excipient.

19. (canceled)

20. A method of treating an inflammatory or autoimmune disease in a subject in need thereof, comprising administering to the subject an effective amount of the compound of claim 1, or a pharmaceutically acceptable salt thereof.

21. The method of claim 20, wherein the inflammatory or autoimmune disease is atopic dermatitis, asthma, lupus, rheumatoid arthritis, familial mediterranean fever, psoriasis, generalized pustular psoriasis, cryoprin-associated periodic syndrome, hidradenitis suppurativa, Bechet's syndrome, or familial cold autoinflammatory syndrome.
